(19) United States
(12) Patent Application Publication
(10) Pub. No.: US 2015/0254400 A1
(43) Pub. Date: Sep. 10, 2015

(54) GROUPING FOR CLASSIFYING GASTRIC CANCER

(71) Applicants: AGENCY FOR SCIENCE, TECHNOLOGY AND RESEARCH, Singapore (SG); NATIONAL UNIVERSITY OF SINGAPORE, Singapore (SG)

(72) Inventors: Zhengdeng Lei, Singapore (SG); Steve Rozen, Singapore (SG); Patrick Tan, Singapore (SG); Manesh Sravanthy, Singapore (SG); Horst Flotow, Singapore (SG); Anna Ngo, Singapore (SG)

(21) Appl. No.: 14/429,283

(22) PCT Filed: Sep. 18, 2013
(86) PCT No.: PCT/SG2013/000409

§ 371 Date: Mar. 18, 2015

(30) Foreign Application Priority Data
Sep. 18, 2012 (SG) 201206943-1

(51) Int. Cl. G06F 19/24; G06F 19/20; A61K 31/513; C12Q 1/68

(52) U.S. Cl. CPC G06F 19/24; C12Q 1/6886; G06F 19/20; A61K 31/513; C12Q 2600/154; C12Q 2600/112; C12Q 2600/16; C12Q 2600/156

(57) ABSTRACT

There is provided a grouping for classifying a gastric cancer tumor sample obtained from a patient suffering or suspected to suffer from gastric cancer, wherein the grouping comprises an invasive subtype, a proliferative subtype and a metabolic subtype. There is also provided a predictor and methods of using the grouping.

### CROSS-REFERENCE TO RELATED APPLICATIONS

This application claims the benefit of priority of Singapore patent application No. 201206943-1, filed Sep. 18, 2012, the contents of it being hereby incorporated by reference in its entirety for all purposes.

### TECHNICAL FIELD

The present invention generally relates to biochemistry and medical applications of biochemical molecules.

### BACKGROUND

Gastric adenocarcinoma (a type of gastric cancer) is the second leading cause of cancer death worldwide, with particularly high incidence and mortality in Eastern Asia, Eastern Europe, and Latin America. Surgery remains the mainstay of treatment but is effective only in early stages. However, due to the lack of symptoms in early stages, most patients are diagnosed with advanced disease and have very poor prognoses.

The underlying mechanisms of gastric carcinogenesis are still poorly understood. Like many other cancers, gastric cancer is heterogeneous, and it arises from and precipitates a multitude of genetic and epigenetic alterations. It exhibits differences between patients in aggressiveness, histopathologic features, and responses to therapy. Consequently, there is a need for genomic taxonomies of gastric cancer that provide insight into oncogenic mechanisms and predict disease behavior and treatment response.

Several histopathological classifications of gastric cancers have been proposed. Among these, the Lauren and WHO systems are widely used. The Lauren classification recognizes two main subtypes, “intestinal” and “diffuse”, which differ in their epidemiology. In parallel with the histology-based classification systems, there is a widely used “tumor-node-metastasis” (TNM) staging system. The TNM system assigns gastric cancers to stages by combining information on size and invasiveness of the primary tumor, the presence and number of lymph node metastases, and the presence or absence of distant, metastases. However, the prognostic value of the histological classification schemes (e.g. Lauren or WHO), in addition to the TNM stages appears to be limited, and it has not been possible to use them as a basis on which to choose particular therapies.

Beyond these histopathological classification schemes, there have been efforts to use microarray-based gene expression profiling to discover new molecular subtypes of gastric cancer by unsupervised clustering. However, these studies relied on relatively small numbers of tumor samples (22 to 47).

In addition, unsupervised clustering on gastric cancer cell lines (as contrasted with surgically removed tumors) was recently carried out. Two subtypes were found that were differentiated by the levels of 171 transcripts. These transcripts then served as the basis for constructing a predictor which was applied to the expression profiles of surgically removed gastric tumors. One of the strengths of this study was that the initial unsupervised clustering was based not on primary tumors consisting of mixtures of malignant and non-malignant cells, but rather on gastric cancer cell lines, with no admixture of non-malignant cells. However, this approach may not have captured the full diversity of gastric cancer subtypes. In particular, it is difficult to derive an immortal cell line from primary gastric tumors. Furthermore, almost all gastric cancer cell lines are derived from metastases or ascites.

There is therefore a need to provide an alternative cancer classification that overcomes, or at least ameliorates, one or more of the disadvantages described above.

### SUMMARY

In a first aspect, there is provided a grouping for classifying a gastric cancer tumor sample obtained from a patient suffering or suspected to suffer from gastric cancer, wherein the grouping comprises an invasive subtype, a proliferative subtype and a metabolic subtype,

wherein the invasive subtype is characterized by any one or more or all of the following:

a. compared to the proliferative subtype and the metabolic subtype, the up-regulated genes in the invasive subtype are associated with any one of the following pathways: focal adhesion, extracellular-matrix-receptor interaction, gap junction, calcium signaling pathway, complement cascades, coagulation cascades, tight junction, regulation of actin cytoskeleton, cell adhesion, vasculature development, blood vessel development, regulation of cell motion, cell motility, extracellular matrix organization, cell-matrix adhesion, angiogenesis, response to wounding, wound healing, and BMP signaling pathway;

b. compared to the proliferative subtype and the metabolic subtype, gene sets that have increased gene set activities in the invasive subtype are selected from the group consisting of: p53, EMT (epithelial-mesenchymal transition), TGF-β, VEGF, NFκB, mTOR, SHH (sonic hedgehog), and CSC (cancer stem cell);

c. compared to the proliferative subtype and the metabolic subtype, invasive subtype tumors are significantly enriched with low-CNA (copy number alteration) tumors;

d. compared to non-malignant tissues, the number of aberrantly methylated CpG sites in the invasive subtype is higher than those in the proliferative subtype and metabolic subtype;

e. the number of aberrantly hypermethylated sites in the invasive subtype is higher than in the proliferative subtype and the metabolic subtype;

f. invasive subtype tumors are not or almost not enriched for TP53 missense mutations compared to the proliferative subtype;

g. the invasive subtype shows strong association to the ‘diffuse’ tumor type according to Lauren classification;

h. compared to the proliferative subtype and the metabolic subtype, the cellular differentiation of invasive subtype tumors is undifferentiated or poorly differentiated;

i. the invasive subtype is more sensitive to compounds targeting the PI3K/AKT/mTOR pathway than in the proliferative and the metabolic subtype; and

j. the invasive subtype shows cancer-stem-cell-like properties;

wherein the proliferative subtype is characterized by any one or more or all of the following:

a. compared to the invasive subtype and the metabolic subtype, the up-regulated genes in proliferative subtype are associated with any one of the following pathways: cell cycle pathway, nuclear division and cell division;

b. compared to the invasive subtype and the metabolic subtype, gene sets that have increased gene set activities in the proliferative subtype are selected from the group consisting of: E2F, MYC, and RAS;

c. compared to the invasive subtype and the metabolic subtype, proliferative subtype tumors are significantly enriched in high-CNA tumors;

d. compared to the invasive subtype and the metabolic subtype, the proliferative subtype is enriched with genomic amplifications of CCNE1, MYC, KRAS, and ERBB2 (also known as HER2);

e. the number of aberrantly hypomethylated CpG sites in the proliferative subtype is higher than in the invasive subtype and the metabolic subtype;

f. proliferative subtype tumors are enriched with hypomethylated CpG sites compared to the invasive subtype and the metabolic subtype;

g. proliferative subtype tumors are enriched with TP53 missense mutations compared to the invasive subtype and the metabolic subtype;

h. the proliferative subtype shows strong association to the ‘intestinal’ tumor type according to Lauren classification; and

i. compared to the invasive subtype and the metabolic subtype, the cellular differentiation of proliferative subtype tumors is well-differentiated or moderately-differentiated;

wherein the metabolic subtype is characterized by any one or more or all of the following:

a. compared to the proliferative subtype and the invasive subtype, the up-regulated genes in metabolic subtype are associated with any one of the following pathways: metabolic processes, digestion and secretion;

b. compared to the invasive subtype and the proliferative subtype, the gene set of spasmolytic polypeptide/(TFF2)-expressing-metaplasia (SPEM) in the metabolic subtype has increased gene set activity;

c. metabolic subtype tumors are not or almost not enriched for TP53 missense mutations compared to the proliferative subtype;

d. metabolic subtype tumors have significantly lower expression of both thymidylate synthase (TS) and dihydropyrimidine dehydrogenase (DPD) transcripts compared to the invasive subtype and the proliferative subtype; and

e. the chance of survival of patients suffering from gastric cancer or suspected to suffer from gastric cancer is higher when treated with adjuvant 5-fluorouracil compared to when undergoing surgery alone.

In a second aspect, there is provided a predictor for classifying a patient based on the gene expression profile to one of the gastric cancer subtypes disclosed herein, wherein the predictor comprises an ensemble of three predictors, wherein each of the three predictors comprises genes that are differentially expressed between one pair of the disclosed subtypes.

In a third aspect, there is provided a method of classifying a patient based on the patient's gene expression profile, wherein the patient is suffering or suspected to suffer from gastric cancer, to one of the gastric cancer subtypes disclosed herein, wherein the method comprises: assigning the gene expression profile obtained from the patient to either the invasive subtype, the proliferative subtype or the metabolic subtype when two of the three disclosed predictors make the same classification and at least one false discovery rate (FDR) is <0.05.

In a fourth aspect, there is provided a method for predicting response to treatment in a patient with gastric cancer, the method comprising assigning the gene expression profile obtained from a gastric tumor sample from the patient to either the invasive subtype, the proliferative subtype or the metabolic subtype disclosed herein when two of the three disclosed predictors make the same classification and at least one false discovery rate (FDR) is <0.05.

In a fifth aspect, there is provided a method of treating a patient suffering or suspected to suffer from gastric cancer, comprising: administering or recommending or prescribing to the patient an anti-cancer drug, or initiating active treatment, specific for the disclosed gastric cancer subtype of the patient.

In a sixth aspect, there is provided a method of treating a patient suffering or suspected to suffer from gastric cancer, comprising: a. determining the gastric cancer subtype of the patient according to the disclosed method of classifying the patient based on the patient's gene expression profile; and b. administering or recommending or prescribing to the patient an anti-cancer drug, or initiating active treatment, specific for the gastric cancer subtype of determined in step a.

In a seventh aspect, there is provided the use of the gastric cancer subtype of a patient suffering or suspected to suffer from gastric cancer determined according to the disclosed method of classifying the patient based on the patient's gene expression profile to recommend or prescribe an anti-cancer drug or to initiate active treatment specific for said gastric cancer subtype.

In an eighth aspect, there is provided a computer readable medium having stored therein a computer program comprising a set of executable instructions, when executed by a computer processor, controls the processor to perform the disclosed method of classifying the patient based on the patient's gene expression profile.

In a ninth aspect, there is provided a computer program comprising a set of executable instructions, when executed by a computer processor, controls the processor to perform the disclosed method of classifying the patient based on the patient's gene expression profile.

### DETAILED DESCRIPTION

Exemplary, non-limiting embodiments of classifying gastric cancer will now be disclosed.

In an embodiment, there is provided a grouping for classifying a gastric cancer tumor sample obtained from a patient suffering or suspected to suffer from gastric cancer, wherein the grouping comprises an invasive subtype, a proliferative subtype and a metabolic subtype,

wherein the invasive subtype is characterized by any one or more or all of the following:

a. compared to the proliferative subtype and the metabolic subtype, the up-regulated genes in the invasive subtype are associated with any one of the following pathways: focal adhesion, extracellular-matrix-receptor interaction, gap junction, calcium signaling pathway, complement cascades, coagulation cascades, tight junction, regulation of actin cytoskeleton, cell adhesion, vasculature development, blood vessel development, regulation of cell motion, cell motility, extracellular matrix organization, cell-matrix adhesion, angiogenesis, response to wounding, wound healing, and BMP signaling pathway;

b. compared to the proliferative subtype and the metabolic subtype, gene sets that have increased gene set activities in the invasive subtype are selected from the group consisting of: p53, EMT (epithelial-mesenchymal transition), TGF-β, VEGF, NFκB, mTOR, SHH (sonic hedgehog), and CSC (cancer stem cell);

c. compared to the proliferative subtype and the metabolic subtype, invasive subtype tumors are significantly enriched with low-CNA (copy number alteration) tumors;

d. compared to non-malignant tissues, the number of aberrantly methylated CpG sites in the invasive subtype is higher than those in the proliferative subtype and metabolic subtype;

e. the number of aberrantly hypermethylated sites in the invasive subtype is higher than in the proliferative subtype and the metabolic subtype;

f. invasive subtype tumors are not or almost not enriched for TP53 missense mutations compared to the proliferative subtype;

g. the invasive subtype shows strong association to the ‘diffuse’ tumor type according to Lauren classification;

h. compared to the proliferative subtype and the metabolic subtype, the cellular differentiation of invasive subtype tumors is undifferentiated or poorly differentiated;

i. the invasive subtype is more sensitive to compounds targeting the PI3K/AKT/mTOR pathway than in the proliferative and the metabolic subtype; and

j. the invasive subtype shows cancer-stem-cell-like properties;

wherein the proliferative subtype is characterized by any one or more or all of the following:

a. compared to the invasive subtype and the metabolic subtype, the up-regulated genes in proliferative subtype are associated with any one of the following pathways: cell cycle pathway, nuclear division and cell division;

b. compared to the invasive subtype and the metabolic subtype, gene sets that have increased gene set activities in the proliferative subtype are selected from the group consisting of: E2F, MYC, and RAS;

c. compared to the invasive subtype and the metabolic subtype, proliferative subtype tumors are significantly enriched in high-CNA tumors;

d. compared to the invasive subtype and the metabolic subtype, the proliferative subtype is enriched with genomic amplifications of CCNE1, MYC, KRAS, and ERBB2 (also known as HER2);

e. the number of aberrantly hypomethylated CpG sites in the proliferative subtype is higher than in the invasive subtype and the metabolic subtype;

f. proliferative subtype tumors are enriched with hypomethylated CpG sites compared to the invasive subtype and the metabolic subtype;

g. proliferative subtype tumors are enriched with TP53 missense mutations compared to the invasive subtype and the metabolic subtype;

h. the proliferative subtype shows strong association to the ‘intestinal’ tumor type according to Lauren classification; and

i. compared to the invasive subtype and the metabolic subtype, the cellular differentiation of proliferative subtype tumors is well-differentiated or moderately-differentiated;

wherein the metabolic subtype is characterized by any one or more or all of the following:

a. compared to the proliferative subtype and the invasive subtype, the up-regulated genes in metabolic subtype are associated with any one of the following pathways: metabolic processes, digestion and secretion;

b. compared to the invasive subtype and the proliferative subtype, the gene set of spasmolytic polypeptide/(TFF2)-expressing-metaplasia (SPEM) in the metabolic subtype has increased gene set activity;

c. metabolic subtype tumors are not or almost not enriched for TP53 missense mutations compared to the proliferative subtype;

d. metabolic subtype tumors have significantly lower expression of both thymidylate synthase (TS) and dihydropyrimidine dehydrogenase (DPD) transcripts compared to the invasive subtype and the proliferative subtype; and

e. the chance of survival of patients suffering from gastric cancer or suspected to suffer from gastric cancer is higher when treated with adjuvant 5-fluorouracil compared to when undergoing surgery alone.

The tumor sample may be obtained from a primary tumor, a secondary tumor or a metastatic tumor. In an embodiment, the tumor sample is obtained from a primary tumor. Advantageously, expression profiles based on primary tumors may capture a more complete array of gastric cancer subtypes, as opposed to cancer cell lines that have no admixture of non-malignant cells.

The tumor sample obtained from the tumor of a patient may be processed to get a gene expression profile. Any suitable processor may be used. For example, DNA microarrays or sequence based techniques may be used. In an example, Affymetrix U133 Plus 2.0 expression arrays were used. The gene expression profile of the sample may then be used as a basis to obtain the grouping subtype.

The term “classify”, and variants thereof, refers to the segregation of data into subcategories. Accordingly, the term “classify” in relation to gastric cancer refers to the segregation of different gene expression profiles of gastric cancer tumors according to subcategories as described herein. There are three subcategories or groups of gastric cancer disclosed herein, namely the invasive subtype, the proliferative subtype and the metabolic subtype. The naming of these groups is derived from the function of the genes that are up-regulated in the particular group.

The terms “gene expression profile” or “gene signature” refer to a group of genes expressed by a particular cell or tissue type wherein presence of the genes or transcriptional products thereof, taken individually (as with a single gene marker) or together or the differential expression of such, is indicative/predictive of a certain condition.

The phrase “suffering from gastric cancer” means that the patient has already been diagnosed with gastric cancer. The phrase “suspected to suffer” refers to a subject that presents one or more symptoms indicative of a cancer (e.g., a noticeable lump or mass). A patient suspected of having cancer may also have one or more risk factors. A patient suspected of having cancer has generally not been tested for cancer. However, a patient suspected of having cancer encompasses an individual who has received an initial diagnosis (e.g., a CT scan showing a mass) but for whom the sub-type or stage of cancer is not known. The term further includes patients who once had cancer (e.g., an individual in remission).

The term “patient” refers to a person suffering or is suspected to suffer from gastric cancer.

Gene transcripts of each subtype are compared against the other two subtypes to determine the genes that are up-regulated in that subtype. It is understood that an up-regulation of genes refers to an increased expression of the genes. Accordingly, an up-regulation of the gene increases the expression of the corresponding protein that the gene encodes for. Conversely, a down-regulation of genes refers to a decreased expression of the genes. Accordingly, a down-regulation of the gene decreases the expression of the corresponding protein that the gene encodes for.

The term “gene” as used herein refers to a polymer in which nucleotides encoding the amino acids constituting a polypeptide (e.g., enzyme) are joined into a linear structure with directionality. The “gene” may be single-stranded (e.g., RNA) or double-stranded (e.g., DNA). DNA may be, for example, cDNA which is enzymatically prepared from a transcribed RNA (mRNA), genomic DNA from chromosomes, or chemically synthesized DNA. Such genes may include a promoter region for regulating the transcription of a coding region, an enhancer region affecting the promoter region, and other regulatory regions (e.g., a terminator and a poly A region) as well as intron or the like, in addition to a sequence corresponding to a coding region or a translational region encoding a polypeptide (e.g., enzyme). It is known in the art that modifications to these genes, e.g., addition, deletion, substitution, may be performed as long as the modified genes retain the activities of the aforementioned regions. The term “gene transcript” is referred to as an RNA transcribed from genomic gene or a cDNA synthesized from this mRNA or can be a non-coding RNA (ncRNA) such as a micro-RNA (miRNA).

The determination of the up-regulation of genes may be analyzed by suitable models known in the art. In an example, the determination of the up-regulation of genes is analyzed by the limma linear model with cutoffs of false discovery rate (FDR) set to less than 0.001 and fold change set to more than 1.5. An FDR corrects for multiple comparisons in a multiple hypothesis testing.

The genes up-regulated in the invasive gastric cancer subtype may be associated with pathways under the Kyoto Encyclopedia of Genes and Genomes (KEGG) database. The genes up-regulated in the invasive gastric cancer subtype may be associated with pathways in the biological process domain under the Gene Ontology (GO) annotations. The up-regulated genes of the invasive subtype may be associated with the following KEGG pathways: focal adhesion, extracellular-matrix-receptor interaction, gap junction, calcium signaling pathway, complement cascades, coagulation cascades, tight junction, regulation of actin cytoskeleton, mitogen-activated protein kinases (MAPK) signaling pathway, and Wnt signaling pathway. The up-regulated genes of the invasive subtype may be associated with the following GO biological process annotations: cell adhesion, vasculature development, blood vessel development, regulation of cell motion, cell motility, extracellular matrix organization, cell-matrix adhesion, angiogenesis, response to wounding, wound healing, and bone morphogenetic proteins (BMP) signaling pathway.

In embodiments, the genes up-regulated in the invasive gastric cancer subtype comprise the genes listed in FIG. 11.

In embodiments, at least 99%, or at least 95%, or at least 90%, or at least 85%, or at least 80% of the genes listed in FIG. 11 may be up-regulated in the invasive gastric cancer subtype.

The genes up-regulated in the proliferative gastric cancer subtype may be associated with pathways under the Kyoto Encyclopedia of Genes and Genomes (KEGG) database. The genes up-regulated in the proliferative gastric cancer subtype may be associated with pathways in the biological process domain under the Gene Ontology (GO) annotations. The up-regulated genes of the proliferative subtype may be associated with the KEGG cell cycle pathway. The up-regulated genes of the proliferative subtype may be associated with the following GO biological process annotations: cell cycle, nuclear division, and cell division.

In embodiments, the genes up-regulated in the proliferative gastric cancer subtype comprise the genes listed in FIG. 12.

In embodiments, at least 99%, or at least 95%, or at least 90%, or at least 85%, or at least 80% of the genes listed in FIG. 12 may be up-regulated in the proliferative gastric cancer subtype.

The genes up-regulated in the metabolic gastric cancer subtype may be associated with pathways under the Kyoto Encyclopedia of Genes and Genomes (KEGG) database. The genes up-regulated in the metabolic gastric cancer subtype may be associated with pathways in the biological process domain under the Gene Ontology (GO) annotations. The up-regulated genes of the metabolic subtype may be associated with various metabolism processes under the KEGG database. The up-regulated genes of the metabolic subtype may be associated with the following GO biological process annotations: digestion and secretion.

In embodiments, the genes up-regulated in the metabolic gastric cancer subtype comprise the genes listed in FIG. 13.

In embodiments, at least 99%, or at least 95%, or at least 90%, or at least 85%, or at least 80% of the genes listed in FIG. 13 may be up-regulated in the metabolic gastric cancer subtype.

The term “pathway” is used herein to refer to a sequence of enzymatic or other reactions by which one biological material is converted to another or by which an effect is achieved.

Gene set activities of each subtype are compared against the other two subtypes to determine the gene sets that are increased in that subtype. The term “gene set” refers to a set of genes, perhaps 5, 10 or more genes, whose pattern of expression in a cell is modulated by a given set of biologically active agents, especially where said agents exert the activity by a common molecular mechanism.

Gene sets may be related to gene expression levels. Specific gene sets may have specific activities. An increased gene set activity refers to an increased expression of the proteins that up-regulated genes in the gene set encode for. Conversely, a decreased gene set activity refers to a decreased expression of the proteins that down-regulated genes in the gene set encode for.

There are several techniques to determine the activity of a gene set. In an example, Bayesian Factor Regression Models (BFRM) can be used. BFRM attempts to model observed gene expression levels as consequences of underlying latent factors that can be viewed as “gene-set activities”. BFRM starts with an initial set of genes that have expression levels partly governed by a particular pathway and then generates a regression model in which the expression levels of these genes are a function of a latent factor (the gene-set activity). The latent factor in the model is related to only a relatively small number of genes related to a specific pathway and therefore, a latent factor model is used in which the factor loadings matrix is sparse. In an example, the maximum number of latent factors is one. Hence, the model can discover the main latent factor which overlays the known biological structure and genes appearing to be linked to a specific pathway. BFRM also refines the model by adding further genes that also appear to be associated with the latent factor.

The invasive subtype may be associated with high gene set activities for the gene sets selected from the group consisting of: p53, EMT (epithelial-mesenchymal transition), TGF-β, VEGF, NFκB, mTOR, SHH (sonic hedgehog), and CSC (cancer stem cell).

Examples of sources of the p53 gene set include target genes up-regulated by p53; and genes up-regulated by expression of p53 in p53-null, brca1-null mouse embryonic fibroblasts (MEFs) and further up-regulated by simultaneous expression of BRCA1. An example of a source of the EMT gene set includes genes up-regulated for epithelial plasticity in tumor progression. An example of sources of the TGF-β gene set includes genes up-regulated by TGF-β treatment of skin fibroblasts. Examples of sources of the VEGF gene set include genes up-regulated after VEGF treatment in human umbilical vein endothelial cells and human myometrial microvascular endothelial cells; and genes overexpressed 3-fold or more in freshly isolated CD31+ and CD31− cells. An example of a source of the NFκB gene set includes genes up-regulated by NFκB. An example of a source of the mTOR gene set includes genes up-regulated in HepaRG cells (liver cancer) expressing constitutively active form of mTOR. An example of a source of the SHH gene set includes genes up-regulated in the activated Hedgehog (Hh) signaling pathway. Examples of sources of the CSC gene set include genes up-regulated in the prostatein and breast cancer stem cell population. In view that the invasive subtype may be associated with high gene set activity for the cancer stem cell (CSC) gene set, the invasive subtype may be shown to have CSC-like properties. Further, as the epithelial-mesenchymal transition confers stem-cell-like properties, the high gene set activity for the related epithelial-mesenchymal transition (EMT) gene set also may indicate that the invasive subtype may be shown to have CSC-like properties.

EMT describes the process driving-epithelial cells to form cells exhibiting a fibroblastic-like morphology (mesenchymal). This mechanism involves multiple steps including the loss of an apico-basolateral polarity. The loss of epithelial cell polarity is induced by the dissolution of junctional complexes (desmosomes and adherens junctions) and tight junctions, and the concomitant remodeling of the actin cytoskeleton. Epithelial cells also delocalize polarity gene products and modulate their integrin adhesome to favor cell substrate adhesions to eventually acquire a mesenchymal phenotype. This critical transdifferentiation program leads to cells with low intercellular adhesion and equipped with rear-front polarity favoring cell locomotion and invasion. In particular, in cancer progression, EMT explains how carcinoma cells invade and metastasize by transforming the epithelial state via an intermediate potentially metastable state to the mesenchymal state.

The proliferative subtype may be associated with high gene set activities for the gene sets selected from the group consisting of: E2F, MYC, and RAS. Examples of sources of the E2F gene set include genes up-regulated by infection with adenovirus expressing activated E2F3; DNA replication genes up-regulated by E2F1 induction; genes up-regulated in hepatoma tissue of Myc+E2f1 transgenic mice and Myc+Tgfa transgenic mice; and genes up-regulated by E2F1 in Saos2 (osteosarcoma). Examples of sources of the MYC gene set include genes up-regulated in hepatoma tissue of Myc transgenic mice and Myc+Tgfa transgenic mice; genes up-regulated by MYC in HUVEC (umbilical vein endothelial cell) and P493-6 (B-cell); genes up-regulated by infection with adenovirus expressing human c-Myc; and other genes up-regulated by MYC. An example of a source of the RAS gene set includes genes up-regulated by infection with adenovirus expressing activated H-Ras.

The metabolic subtype may be associated with high gene set, activities for the gene set of spasmolytic polypeptide/(TFF2)-expressing-metaplasia (SPEM). An example of a source of the SPEM gene set includes genes up-regulated in SPEM.

Copy-number alteration (CNA) refers to alterations of the deoxyribonucleic acid (DNA) of a genome that result in the cell having an abnormal number of copies of one or more sections of the DNA. CNA may be due to large-scale genomic deletions, duplications and amplifications.

Invasive subtype tumors may be significantly enriched with low-CNA tumors when compared with the other subtypes.

Proliferative subtype tumors may be enriched with more CNA gain than CNA loss. Thus, proliferative subtype tumors may be significantly enriched with high-CNA tumors when compared with the other subtypes. Proliferative subtype tumors may also be enriched for genomic amplifications of CCNE1, MYC, KRAS, and ERBB2 (also known as HER2).

The term “enriched” in reference to a property of a tumor belonging to a particular subtype means that the specific property constitutes a significantly higher fraction (about 2 to 5 fold or more) of the tumor than in a tumor belonging to another subtype, unless otherwise specified. However, it should be noted that “enriched” does not imply that there are no other properties present, just that the relative amount of the property of interest has been significantly increased when compared to other properties.

The term “significant” in the context of the specification generally means an increase in a specific property relative to other properties of about at least 2 fold, at least 5 to 10 fold or more, unless otherwise specified. The term also does not imply that the increase in the specified property does not come from other sources.

“CpG” refers to a region of DNA where a cytosine (C) nucleotide occurs next to a guanine (G) nucleotide, separated by one linker phosphate (p), in the linear sequence of bases along its length.

Aberrant methylation of CpG sites can lead to malignancies. Hence, aberrantly methylated CpG sites of each subtype may be compared against non-malignant tissues. The methylation levels of each CpG site in tumors of each subtype may be compared against methylation levels in non-malignant samples. The difference between methylation levels of malignant tissues and non-malignant tissues may be used to indicate aberrant methylation. In an embodiment, significant hyper- or hypomethylated CpG sites in tumors indicate aberrant methylation. In this embodiment, “significant” may be identified using t-tests, e.g. two sided t-tests, with a Bonferroni corrected alpha. In an example, the Bonferroni corrected alpha is 0.05/26,486. The terms “hypomethylation” and “hypermethylation”, or variants thereof, are relative terms and denote less or more methylation, respectively, than in non-malignant tissues.

In embodiments, the invasive subtype has the highest number of aberrantly methylated CpG sites, e.g. more than 10%, when compared with non-malignant tissues. In an embodiment, the invasive subtype has about 11.1% of aberrantly methylated CpG sites compared to non-malignant tissues. In another embodiment, the invasive subtype has a higher number of aberrant methylated CpG sites than those in the other subtypes, compared to non-malignant tissues. In embodiments, the aberrant methylation of the invasive subtype is an aberrant hypermethylation. Invasive subtype tumors may be significantly enriched for hypermethylated sites. The number of aberrantly hypermethylated sites may also be higher than those in the other subtypes.

In embodiments, the proliferative subtype has between 5-10% of aberrantly methylated CpG sites when compared with non-malignant tissues. In an embodiment, the proliferative subtype has about 9.3% of aberrantly methylated CpG sites compared to non-malignant tissues. In embodiments, the aberrant methylation of the proliferative subtype is an aberrant hypomethylation. Proliferative subtype tumors may be significantly enriched for hypomethylated CpG sites. The number of aberrantly hypomethylated CpG sites may also be higher than those in the other subtypes.

In embodiments, the metabolic subtype has less than 5% of aberrantly methylated CpG sites when compared with non-malignant tissues. In an embodiment, the metabolic subtype has about 4.1% of aberrantly methylated CpG sites compared to non-malignant tissues. The metabolic subtype may not be considered significantly enriched in hyper- or hypomethylated sites when applying the t-tests with a Bonferroni corrected alpha.

CpG sites showing aberrant hyper- and hypomethylation in one subtype when compared to the other two subtypes constitute a methylation signature of that subtype. The methylation signature indicates that the subtype is enriched with particular aberrantly methylated CpG sites. The gene nearest to the aberrantly methylated CpG site is annotated by function, e.g. a pathway or an interaction.

The hypermethylation signature of the invasive subtype may be associated with pathways under the KEGG database. The hypermethylation signature of the invasive subtype may be associated with the KEGG focal adhesion and apoptosis pathways. The hypomethylation signature of the invasive subtype may be associated with focal adhesion.

In embodiments, the hypermethylated CpG sites of the invasive subtype comprise the genes listed in FIG. 14. In embodiments, at least 99%, or at least 95%, or at least 90%, or at least 85%, or at least 80% of the genes listed in FIG. 14 may be hypermethylated in the invasive subtype.

In embodiments, the hypomethylated CpG sites of the invasive subtype comprise the genes listed in FIG. 15. In embodiments, at least 99%, or at least 95%, or at least 90%, or at least 85%, or at least 80% of the genes listed in FIG. 15 may be hypomethylated in the invasive subtype.

The hypermethylation signature of the proliferative subtype may be associated with neuroactive ligand-receptor interaction. The hypomethylation signature of the proliferative subtype may be associated with cytokine-cytokine receptor interaction- and Jak-STAT signaling pathways.

In embodiments, the hypermethylated CpG sites of the proliferative subtype comprise the genes listed in FIG. 16. In embodiments, at least 99%, or at least 95%, or at least 90%, or at least 85%, or at least 80% of the genes listed in FIG. 16 may be hypermethylated in the proliferative subtype.

In embodiments, the hypomethylated CpG sites of the proliferative subtype comprise the genes listed in FIG. 17. In embodiments, at least 99%, or at least 95%, or at least 90%, or at least 85%, or at least 80% of the genes listed in FIG. 17 may be hypomethylated in the proliferative subtype.

As the metabolic subtype may not be significantly enriched in hyper- or hypomethylated sites, the metabolic subtype may not have a methylation signature.

The determination of the methylation signature may be analyzed by suitable models known in the art. In an example, the determination of the methylation signature is analyzed by the limma linear model with cutoffs of false discovery rate (FDR) set to less than 0.01 and absolute; β-value difference set to more than 0.1.

The methylation signature may be obtained by determining the CpG sites of each subtype that were aberrantly methylated in the respective subtype. In an embodiment, a hypomethylation signature is obtained by determining the CpG sites of a subtype that were hypomethylated in that subtype. In another embodiment, a hypermethylation signature is obtained by determining the CpG sites of a subtype that were hypermethylated in that subtype.

Mutation of the TP53 gene is a characteristic of tumors. In particular, exons 4-9 of the TP53 gene are mutation hotspots. The sequence for exon 4 (including flanks) of the human TP53 gene is represented by SEQ ID NO: 1, the sequence for exons 5 and 6 (including flanks) of the human TP53 gene is represented by SEQ ID NO: 2, the sequence for exon 7 (including flanks) of the human TP53 gene is represented by SEQ ID NO: 3 and the sequence for exons 8 and 9 (including flanks) of the human TP53 gene is represented by SEQ ID NO: 4. The vast majority of cancer-associated mutations in TP53 are missense mutations, single base-pair substitutions that, result in the translation of a different amino acid in that position in the context of the full-length protein. In embodiments, proliferative-subtype tumors have an increased amount of TP53 missense mutations compared to the other subtypes. In other embodiments, proliferative-subtype tumors are enriched for TP53 missense mutations compared to the other subtypes. In embodiments, the invasive and metabolic subtypes are not or almost not enriched, e.g. less than 50% or less than 40% or less than 30% or less than 20%, for TP53 missense mutations compared to the proliferative subtype.

In an example, based on Fisher's exact hypergeometric test and a p-value of 3.16×10⁻³, a sample of 124 tumors yielded results summarized in Table 1 below. As can be seen, the invasive and metabolic subtypes are 25% and 33%, respectively, less enriched for TP53 missense mutations compared to the proliferative subtype.

TABLE 1

| TP53 mutation status | Invasive | Proliferative | Metabolic |
|---|---|---|---|
| Wild type | 30 | 30 | 26 |
| Mutated | 6 | 24 | 8 |

The three subtypes may have significant differences with respect to the Lauren classification. In embodiments, the invasive subtype shows strong association to the diffuse-type gastric tumors as compared to intestinal-type gastric tumors according to the Lauren Classification. In embodiments, the proliferative subtype shows strong association to the intestinal-type gastric tumors as compared to diffuse-type gastric tumors according to the Lauren Classification. In embodiments, the metabolic subtype does not show strong association to a particular type of gastric tumor according to the Lauren Classification.

The disclosed subtypes may have significant differences with respect to the level of cellular differentiation. Grading is measure of the cell appearance in tumors. Low-grade cancers are well-differentiated, intermediate-grade cancers are moderately-differentiated and high-grade cancers are poorly or undifferentiated.

In embodiments, proliferative subtype tumors are low-grade or intermediate-grade tumors as compared to the invasive subtype and the metabolic subtype. That is, proliferative subtype tumors are well-differentiated or moderately differentiated as compared to the invasive subtype and the metabolic subtype.

In embodiments, invasive subtype tumors are high-grade tumors as compared to the other subtypes. That is, invasive subtype tumors may be undifferentiated or poorly differentiated as compared to the other subtypes. Maintenance of an undifferentiated state is an essential characteristic of cancer stem cells. Accordingly, the invasive subtype may show cancer-stem-cell-like properties.

In embodiments, invasive subtype gastric tumors have high CD44 and low CD24 expression compared to the other subtypes. Here, “high” and “low” may be quantified using t-tests, e.g. two sided t-tests. In an embodiment, p-values of 1.17e-5 for CD44 and 3.39e-9 for CD24 are used. This pattern of CD44 and CD24 expression has been observed in quasi-mesenchymal pancreatic ductal adenocarcinomas, and has been used to fractionate CSCs in breast cancer and pancreatic cancer. CD44 and CD24 have also been associated with invasiveness and metastasis of breast cancer. Therefore, the CD44 and CD24 expression indicates that the invasive subtype may show cancer-stem-cell-like properties.

In embodiments, the three subtypes may have no significant differences with respect to tumor site, TNM stage, cancer recurrence, patient age, patient gender, active Helicobacter pylori infection, or microsatellite instability. The tumor sites refer to the upper, middle and/or lower parts of the gastric system. Examples of tumor sites in the upper part of the gastric system are the Cardia, Fundus, Gastroesophageal (GE) junction and Incisura sites. Examples of tumor sites in the middle part of the gastric system are the body, greater curve and lesser curve of the stomach. Examples of tumor sites in the lower part of the gastric system are the Pylorus and Antrum sites.

The invasive subtype may correspond to the G-DIF intrinsic genomic subtype of gastric cancer, while the metabolic subtype may correspond to the G-INT intrinsic genomic subtype of gastric cancer. The proliferative subtype may have no significant correspondence with respect to either the G-INT or G-DIF intrinsic genomic subtype of gastric cancer.

In embodiments, there may be no significant survival difference between the three subtypes. Here, “significant” may be based on a Kaplan-Meier analysis. In an embodiment, the statistical significance is calculated by the log-rank test used with a p of 0.310. In embodiments where patients in all three subtypes have high TNM stages, metabolic subtype patients survive better when treated with adjuvant 5-fluorouracil based therapy as compared to patients of the other subtypes. In other embodiments, metabolic subtype patients survive better when treated with adjuvant 5-fluorouracil based therapy compared to when undergoing surgery alone.

In embodiments, the invasive subtype is significantly more sensitive to compounds that inhibit the PI3K/AKT/mTOR pathway. The PI3K/AKT/mTOR pathway regulates cellular metabolism, proliferation and survival. The term “more sensitive” refers to the IC50 value of the compounds that target the PI3K/AKT/mTOR pathway that are significantly lower, when compared with cells in the other subtypes.

Examples of compounds that target the PI3k pathway are 2-Methyl-2-{4-[3-methyl-2-oxo-8-(quinolin-3-yl)-2,3-dihydro-1H-imidazo[4,5-c]quinolin-1-yl]phenyl}propanenitrile (BEZ235), 4,4′-(6-(2-(difluoromethyl)-1H-benzo[d]imidazol-1-yl)-1,3,5-triazine-2,4-diyl)dimorpholine (ZSTK474), (E)-N′-((6-brombimidazo[1,2-a]pyridin-3-yl)methylene)-N,2-dimethyl-5-nitrobenzenesulfonohydrazide hydrochloride (PIK-75), 3-[4-(4-Morpholinylpyrido[3′,2′:4,5]furo[3,2-d]pyrimidin-2-yl]phenol hydrochloride (PI-103), and PI-103 hydrochloride. Other examples of PI3K inhibitors include 1,1-Dimethylpiperidinium-4-yl octadecyl phosphate (perifosine), 5-fluoro-3-phenyl-2-([S)]-1-[9H-purin-6-ylamino]-propyl)-3H-quinazolin-4-one (CAL101), acetic acid (1S,4E,10R,11R,13S,14R)-[4-diallylaminomethylene-6-hydroxy-1-methoxymethyl-10,13-dimethyl-3,7,17-trioxo-1,3,4,7,10,11,12,13,14,15,16,17-dodecahydro-2-oxa-cyclopenta[a]phenanthren-11-yl ester (PX-866), (S)-3-(1-9H-purin-6-yl)amino)ethyl)-8-chloro-2-phenylisoquinolin-1(2H)-one (IPI-145), 2-amino-N-(7-methoxy-8-(3-morpholinopropoxy)-2,3-dihydroimidazo[1,2-c]quinazolin-5-yl)pyrimidine-5-carboxamide (BAY 80-6946), RP6503, TGR 1202, (8S,14S,17S)-14-(carboxymethyl)-8-(3-guanidinopropyl)-17-(hydroxymethyl)-3,6,9,12,15-pentaoxo-1-(4-(4-oxo-8-phenyl-4H-chromen-2-yl)morpholino-4-ium)-2-oxa-7,10,13,16-tetraazaoctadecan-18-oate (SF1126), INK1117, 2-(1H-indazol-4-yl)-6-(4-methanesulfonyl-piperazin-ylmethyl)-4-morpholin-4-yl-thieno[3,2-d]pyrimidine, bimesylate salt (GDC-0941), 5-(2,6-dimorpholinopyrimidin-4-yl)-4-(trifluoromethyl)pyridin-2-amine (BKM120), N-(3-(benzo[c][1,2,5]thiadiazol-5-ylamino) quinoxalin-2-yl)-4-methylbenzenesulfonamide (XL147), N-(4-(N-(3-((3,5-dimethoxyphenyl)amino)quinoxalin-2-yl)sulfamoyl)phenyl) 3-methoxy-4-methylbenzamide (XL765), 8-(1-hydroxyethyl)-2-methoxy-3-((4-methoxybenzyl)oxy)-6H-benzo[c]chromen-6-one (Palomid 529), 5-[[4-(4-Pyridinyl)-6-quinolinyl]me-thylene]-2,4-thiazolidenedione (GSK1059615), PWT33597, 2-((6-amino-9H-purin-9-yl)methyl)-5-methyl-3-o-tolylquinazolin-4(3H)-one (IC87114), 3-[2,4-diamino-6-(3-hydroxyphenyl) pteridin-7-yl]phenol (6,7-Bis(3-hydroxyphenyl)pteridine-2,4-diamine) (TG100-115), CAL263, RP6530, GNE-477, CUDC-907 and AEZS-136.

An example of a compound that targets the mTOR pathway is 2-Methyl-2-{4-[3-methyl-2-oxo-8-(quinolin-3-yl)-2,3-dihydro-1H-imidazo[4,5-c]quinolin-1-yl]phenyl}propanenitrile (BEZ235). Other examples of mTOR inhibitors include (3S,6R,7E,9R,10R,12R,14S,15E,17E,19E,21S,23S,26R,27R,34aS)-9,10,12,13,14,21,22,23,24,25,26,27,32,33,34,34a-hexadecahydro-9,27-dihydroxy-3-[(1R)-2-[(1S,3R,4R)-4-hydroxy-3-methoxycyclohexyl]-1-methylethyl]-10,21-dimethoxy-6,8,12,14,20,26-hexamethyl-23,27-epoxy-3H-pyrido[2,1-c][1,4]-oxaazacyclohentriacontine-1,5,11,28,29(4H,6H,31H)-pentone (rapamycin), (2R,3R)-5,7-dihydroxy-2-(3,4,5-trihydroxyphenyl)-3,4-dihydro-2H-1-benzopyran-3-yl 3,4,5-trihydroxybenzoate (epigallocatechin gallate), caffeine, curcumin, resveratrol, (1R,2R,4S)-4-{(2R)-2-[(3S,6R,7E,9R,10R,12R,14S,15E,17E,19E,21S,23S,26R,27R,34aS)-9,27-dihydroxy-10,21-dimethoxy-6,8,12,14,20,26-hexamethyl-1,5,11,28,29-pentaoxo-1,4,5,6,9,10,11,12,13,14,21,22,23,24,25,26,27,28,29,31,32,33,34,34a-tetracosahydro-3H-23,27-epoxypyrido[2,1-c][1,4]oxazacyclohentriacontin-3-yl]propyl}-2-methoxycyclohexyl 3-hydroxy-2-(hydroxymethyl)-2-methylpropanoate (temsirolimus), dihydroxy-12-[(2R)-1-[(1S,3R,4R)-4-(2-hydroxyethoxy)-3-methoxycyclohexyl]propan-2-yl]-19,30-dimethoxy-15,17,21,23,29,35-hexamethyl-11,36-dioxa-4-azatricyclo[30.3.1.0^{4,9}]hexatriaconta-16,24,26,28-tetraene-2,3,10,14,20-pentone (everolimus) and (1R,2R,4S)-4-[(2R)-2-[(1R,9S,12S,15R,16E,18R,19R,21R,23S,24E,26E,28Z,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.0^{4,9}]hexatriaconta-16,24,26,28-tetraen-12-yl]propyl]-2-methoxycyclohexyl dimethylphosphinate (Ridaforolimus).

Examples of compounds that target the AKT pathway are (S)-4-(2-(4-amino-1,2,5-oxadiazol-3-yl)-1-ethyl-7-(piperidin-3-ylmethoxy)-1H-imidazo[4,5-c]pyridin-4-yl)-2-methylbut-3-yn-2-ol (GSK690693), and 8-(4-(1-aminocyclobutyl)phenyl)-9-phenyl-8,9-dihydro-[1,2,4]triazolo[3,4-f][1,6]naphthyridin-3(2H)-one dihydrochloride (MK2206). Another example of a compound that the invasive subtype may be significantly more sensitive to is 6-(2,6-dichlorophenyl)-8-methyl-2-((3-(methylthio)phenyl)amino)pyrido[2,3-d]pyrimidin-7(8H)-one (PD173955). Other examples of AKT inhibitors include 1,1-Dimethylpiperidinium-4-yl octadecyl phosphate (Perifosine), 2-(hexadecoxy-oxido-phosphoryl)oxyethyl-trimethyl-azanium (Miltefosine) and (S)-4-amino-N-(1-(4-chlorophenyl)-3-hydroxypropyl)-1-(7H-pyrrolo[2,3-d]pyrimidin-4-yl)piperidine-4-carboxamide (AZD5363).

Glioblastoma and prostate cancer stem cells are known to display preferential sensitivity to PI3K/AKT/mTOR inhibitors. Therefore, the invasive subtype being sensitive to PI3K/AKT/mTOR inhibitors indicates that the invasive subtype may show cancer-stem-cell-like properties.

As described in various embodiments above, the cancer-stem-cell-like properties may be characterized a) in that a pathway activity analysis shows that invasive subtype cancers are associated with activity of a cancer-stem-cell (CSC) gene set and with epithelial-mesenchymal transition conferring stem-cell-like properties; b) that CD44 expression is increased and CD24 expression is decreased compared to the proliferative subtype and the metabolic subtype; c) that it is associated with high-grade (that is, undifferentiated or poorly differentiated) gastric cancers; and d) that it is sensitive to compounds inhibiting the PI3K/AKT/mTOR pathway.

As used in the context of the specification, the phrase “inhibiting the PI3K/AKT/mTOR pathway”, or variants thereof, means that the activity of the PI3K, AKT and/or mTOR proteins is decreased or absent. Further, the phrase “inhibiting the PI3K/AKT/mTOR pathway”, or variants thereof, is not particularly limited and may also encompass the inhibition of the PI3K/AKT/mTOR genes. Inhibiting the PI3K/AKT/mTOR genes means that the expression of the PI3K, AKT and/or mTOR genes is decreased or absent. “Absent” means that there is completely no expression of the PI3K, AKT and/or mTOR genes or activity of the PI3K, AKT and/or mTOR proteins. It is understood that the inhibition of the PI3K, AKT and/or mTOR genes decreases the expression of the PI3K, AKT and/or mTOR proteins.

In embodiments, metabolic subtype tumors have significantly lower expression of both thymidylate synthase (TS) and dihydropyrimidine dehydrogenase (DPD) transcripts compared to the invasive subtype and the proliferative subtype.

The biological and clinical characteristics of the three subtypes of a type of gastric cancer, i.e. gastric adenocarcinoma, are summarized in Table 2 based on 248 expression profiles. Out of the total 248 expression profiles, Table 3 summarizes 201 samples that have an average consensus index of more than 0.9 as representative of their clusters.

The average consensus index of a sample is defined as the average of its consensus indices vis-à-vis samples in the same cluster (i.e. in the same dark block in the consensus matrix, FIG. 2E referred to in Example 1). For a sample with completely stable cluster assignments, the average consensus index is 1.

TABLE 2

| | Invasive | Proliferative | Metabolic |
|---|---|---|---|
| 5-FU effect on patient | No effect | No effect | Beneficial |
| survival | | | |
| Chemosensitivity in | PI3K/AKT/mTOR inhibitors | — | 5-FU |
| cell lines | | | |
| KEGG pathways | Focal adhesion, ECM-receptor | Cell cycle | Metabolic |
| associated with up- | interaction, gap junction, calcium | | processes |
| regulated genes | signaling pathway, complement and | | |
| | coagulation cascades, tight | | |
| | junction, and regulation of actin | | |
| | cytoskeleton | | |
| Gene Ontology | Cell adhesion, vasculature | Cell cycle, | Digestion, |
| biological processes | development, blood vessel | nuclear | secretion |
| associated with up- | development, regulation of cell | division, cell | |
| regulated genes | motion, cell motility, extracellular | division | |
| | matrix organization, cell-matrix | | |
| | adhesion, angiogenesis, response to | | |
| | wounding, wound healing, BMP | | |
| | signaling pathway | | |
| Activated gene sets | p53, EMT, TGF-β, VEGF, NFκB, | E2F, MYC, | SPEM |
| | mTOR, SHH, and CSC | and RAS | |
| Grade | High | Low | — |
| TNM Stage | No significant difference among the three subtypes | | |
| Age ± SD | 62.51 ± 12.33 | 66.42 ± 11.78 | 63.28 ± 15.71 |
| Lauren Classification (%) | | | |
| Diffuse | 58.2 | 17.3 | 40.6 |
| Intestinal | 29.9 | 73.6 | 53.6 |
| Mixed | 11.9 | 9.1 | 5.8 |
| Classification in (Tan et al., 2011) (%) | | | |
| G-INT | 7.5 | 71.2 | 84.3 |
| G-DIF | 92.5 | 28.8 | 15.7 |
| Characteristic copy | Low CNA | High CNA | — |
| number alteration | | | |
| Amplified Genes | — | MYC, | — |
| | | ERBB2, | |
| | | KRAS | |
| Aberrantly methylated CpGs (%) | | | |
| Hypermethylated | 84.6 | 57.8 | 76.1 |
| Hypomethylated | 15.4 | 42.2 | 23.9 |
| Characteristic | Hypermethylation | Hypo- | — |
| aberrant methylation | | methylation | |
| Frequency of TP53 | Low | High | Low |
| mutation | | | |

TABLE 3

| | Invasive | Proliferative | Metabolic | P-value |
|---|---|---|---|---|
| Lauren Classification | | | | 3.97e−8 |
| Diffuse | 35 | 14 | 17 | |
| Intestinal | 17 | 70 | 28 | |
| Mixed | 8 | 8 | 2 | |
| Tumor Grade | | | | 1.55e−3 |
| Low | 13 | 45 | 19 | |
| High | 47 | 44 | 29 | |
| Age (mean ± sd) | 62.51 ± 12.33 | 66.42 ± 11.78 | 63.28 ± 15.71 | 0.211 |
| Tumor site | | | | 0.084 |
| Upper | 5 | 17 | 3 | |
| Middle | 26 | 32 | 24 | |
| Lower | 18 | 23 | 8 | |
| Gender | | | | 0.531 |
| Female | 22 | 28 | 19 | |
| Male | 38 | 64 | 29 | |
| Recurrence | | | | 0.458 |
| Yes | 29 | 35 | 18 | |
| No | 30 | 51 | 30 | |
| Stage | | | | 0.529 |
| 1 | 8 | 15 | 11 | |
| 2 | 10 | 15 | 9 | |
| 3 | 19 | 38 | 14 | |
| 4 | 23 | 24 | 13 | |
| H. Pylori status | | | | 0.390 |
| Yes | 22 | 27 | 13 | |
| No | 12 | 27 | 12 | |
| Microsatellite status | | | | 0.827 |
| MSI | 0 | 3 | 2 | |
| MSS | 6 | 17 | 9 | |
| G-INT/G-DIF | | | | <2.2e−16 |
| G-INT | 4 | 67 | 45 | |
| G-DIF | 56 | 26 | 3 | |

All by Fisher's exact test, except for age by ANOVA

In embodiments, there is provided a predictor for classifying a patient based on the gene expression profile to one of the disclosed gastric cancer subtypes, wherein the predictor comprises an ensemble of three predictors, wherein each of the three predictors comprises genes that are differentially expressed between one pair of the disclosed subtypes.

The predictor enables the forecast of the cancer subtype of a patient.

A relatively large number of expression profiles from tumor samples, e.g. more than 100, or more than 150, or more than 200 may be used to build the ensemble. In an embodiment, 248 expression profiles are used.

The expression profiles of the ensemble may be processed by a suitable prediction approach. In an example, Nearest Template Prediction (NTP) is used. Advantageously, this approach is robust to differences in experimental and analytical conditions. Thus, it is suitable for gene-expression-based classification of samples as they arrive one-by-one over time. Another advantage of NTP is that it provides a measure of prediction confidence.

In an embodiment, the ensemble has three predictors. Each of the three predictors may comprise genes that are differentially expressed between one pair of the disclosed subtypes. Each predictor may be based on the NTP approach. To determine the genes to be comprised in each predictor, the top differentially expressed genes between a chosen pair of subtypes may be obtained by analyzing the genes with suitable models known in the art. In an example, the determination of the top differentially expressed genes between a chosen pair of subtypes is analyzed by the limma linear model with cutoffs of false discovery rate (FDR) set to less than 0.001 and absolute fold change set to more than 1.5.

The gene signatures of each subtype and t-scores then served as the features and weights in the constituent NTPs. The t-score refers to moderated t statistics output from limma. A higher t-score means that the gene has a higher differential expression between the chosen pair of subtypes.

All differentially expressed genes contribute to determining the subtype of a test sample. In embodiments, the first predictor comprises genes differentially expressed between the invasive subtype and the proliferative subtype, the second predictor comprises genes differentially expressed between the invasive subtype and the metabolic subtype and the third predictor comprises genes differentially expressed between the proliferative subtype and the metabolic subtype.

In embodiments, the genes with higher t-scores have a higher weightage in the predictor, resulting in a greater influence in determining the subtype.

In embodiments, the first predictor comprises a differentially expressed gene set comparing the differential expression between genes of the invasive subtype versus the proliferative subtype as shown in FIG. 18. The positive t-scores shown in FIG. 18 indicate the genes from the invasive subtype that are up-regulated as compared to the same genes from the proliferative subtype, while the negative t-scores shown in FIG. 18 indicate the genes from the proliferative subtype that are up-regulated as compared to the same genes from the invasive subtype. In embodiments, the first predictor comprises at least a portion of the genes listed in FIG. 18. The first predictor may comprise at least the genes listed in FIG. 18 that have an absolute t-score of more than 3.

In embodiments, the second predictor comprises a differentially expressed gene set comparing the differential expression between genes of the invasive subtype versus the metabolic subtype as shown in FIG. 19. The positive t-scores shown in FIG. 19 indicate the genes from the invasive subtype that are up-regulated as compared to the same genes from the metabolic subtype, while the negative t-scores shown in FIG. 19 indicate the genes from the metabolic subtype that are up-regulated as compared to the same genes from the invasive subtype. In embodiments, the second predictor comprises at least a portion of the genes listed in FIG. 19. The second predictor may comprise at least the genes listed in FIG. 19 that have an absolute t-score of more than 3.

In embodiments, the third predictor comprises a differentially expressed gene set comparing the differential expression between genes of the proliferative subtype versus the metabolic subtype as shown in FIG. 20. The positive t-scores shown in FIG. 20 indicate the genes from the proliferative subtype that are up-regulated as compared to the same genes from the metabolic subtype, while the negative t-scores shown in FIG. 20 indicate the genes from the metabolic subtype that are up-regulated as compared to the same genes from the proliferative subtype. In embodiments, the third predictor comprises at least a portion of the genes listed in FIG. 20. The third predictor may comprise at least the genes listed in FIG. 20 that have an absolute t-score of more than 3.

In embodiments, there is provided a kit comprising the predictor defined herein.

In embodiments, there is provided a method for predicting response to treatment in a patient with gastric cancer, the method comprising assigning the gene expression profile obtained from a gastric tumor sample from the patient to either the invasive subtype, the proliferative subtype or the metabolic subtype disclosed herein when two of the three predictors disclosed herein make the same classification and at least one false discovery rate (FDR) is <0.05.

When the gene expression profile is assigned to the metabolic subtype, the patient may be responsive to 5-fluorouracil.

When the gene expression profile is assigned to the invasive subtype, the patient may be responsive to compounds selected to inhibit the PI3K/AKT/mTOR pathway.

In embodiments, there is provided a computer readable medium having stored therein a computer program comprising a set of executable instructions, when executed by a computer processor, controls the processor to perform the method of classifying a patient to one of the disclosed subtypes.

In embodiments, there is provided a computer program comprising a set of executable instructions, when executed by a computer processor, controls the processor to perform the method of classifying a patient to one of the disclosed subtypes.

The computer program may store information of a grouping for classifying a gastric cancer tumor sample as disclosed herein. The computer program may be stored in a microarray system. When a new sample is processed by a microarray, the instructions in the computer, program may assign the new sample to a cancer subtype as disclosed herein.

In embodiments, there is provided a method of classifying a patient based on the patient's gene expression profile, wherein the patient is suffering or suspected to suffer from gastric cancer, to one of the disclosed gastric cancer subtypes, wherein the method comprises: assigning the gene expression profile obtained from the patient to either the invasive subtype, the proliferative subtype or the metabolic subtype when two of the three predictors' defined herein make the same classification and at least one false discovery rate (FDR) is <0.05.

When neither FDR is <0.05 or when all three predictors make different classifications, the gene expression profile sample is not classified.

In embodiments, there is provided a method of treating a patient suffering or suspected to suffer from gastric cancer, comprising: administering or recommending or prescribing to the patient an anti-cancer drug, or initiating active treatment, specific for the gastric cancer subtype of the patient disclosed herein.

In other embodiments, there is provided a method of treating a patient suffering or suspected to suffer from gastric cancer, comprising: a. determining the gastric cancer subtype of the patient according to the disclosed method of classifying a patient to one of the disclosed subtypes; and b. administering or recommending or prescribing to the patient an anti-cancer drug, or initiating active treatment, specific for the gastric cancer subtype of determined in step a.

In embodiments, there is provided the use of the gastric cancer subtype of a patient suffering or suspected to suffer from gastric cancer determined according to the method of classifying a patient to one of the disclosed subtypes to recommend or prescribe an anti-cancer drug or to initiate active treatment specific for said gastric cancer subtype.

Where the gastric cancer subtype is determined to be the metabolic subtype, the anti-cancer drug may be 5-fluorouracil.

Where the gastric cancer subtype is determined to be the invasive subtype, the anti-cancer drug may be selected to inhibit the PI3K/AKT/mTOR pathway.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The word “substantially” does not exclude “completely” e.g. a composition which is “substantially free” from Y may be completely free from Y. Where necessary, the word “substantially” may be omitted from the definition of the invention.

As used herein, the term “about”, in the context of concentrations of components of the formulations, typically means+/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### BRIEF DESCRIPTION OF DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1 shows a flowchart of the process involved in consensus clustering in combination with iterative feature selection used in Example 1.

FIGS. 2A and 2B show the cumulative distribution function (CDF) of the consensus indices referred to in Example 1. As the number of clusters increases, the area under the CDF increases appreciably if a larger proportion of sample pairs have consensus indices near zero, indicating that they are well-separated by the clustering. At a certain point, however, increasing the number of clusters does not appreciably increase the area under the CDF. This is either because some of the new clusters have very few members or because membership in some of the new clusters is unstable, resulting in consensus indices intermediate between 0 and 1. FIGS. 2C and 2D show the relative increase in the area under the CDF curves as K increases and indicate that there are three clusters. FIGS. 2E and 2F show the consensus matrix indices together constitute the consensus matrix.

FIG. 3 shows the consensus matrices constructed with and without iterative feature selection referred to in Example 1. (A) and (C) show the consensus matrix and distribution of average consensus index without iterative feature selection, respectively. (B) and (D) show the consensus matrix and distribution of average consensus index with iterative feature selection, respectively. The sharper definition of (B) and a high average consensus index of 0.1 in (D) show that the iterative feature selection improve clustering stability.

FIG. 4 shows the genomes of tumors having CNA referred to in Example 2. FIG. 4A evidences that proliferative tumors were found to have significantly more CNA than the other two subtypes. FIG. 4B evidences that invasive-subtype tumors are significantly enriched for low-CNA tumors and proliferative-subtype tumors are significantly enriched for high-CNA tumors, while metabolic tumors are enriched in neither.

FIG. 5 shows that enrichment for high-CNA tumors in the proliferative subtype is primarily due to copy number gains. FIG. 5A shows that there is a significant difference of median value of cytobands with copy number gains among the three subtypes and FIG. 5B shows that there is a much less significant difference of median value of cytobands with copy number loss among the three subtypes.

FIG. 6 shows that invasive-subtype tumors are significantly enriched for hypermethylated sites, while proliferative-subtype tumors are significantly enriched for hypomethylated sites and metabolic-subtype tumors are enriched for neither.

FIG. 7 shows the Kaplan-Meier analysis of the Singapore cohort referred to in Example 4, evidencing that there is no significant survival difference among the three subtypes, although there is a trend for invasive-subtype patients to have worse survival.

FIGS. 8A to F show Kaplan-Meier plots for surgery plus 5-FU versus surgery alone for each of the three subtypes referred to in Example 4. It is evidenced that patients with metabolic subtypes benefited from 5-FU treatment and surgery.

FIG. 9 shows a Kaplan-Meier analysis with disease-free survival as the endpoint shows that metabolic-subtype patients benefited significantly from 5-FU treatment compared to surgery alone.

FIG. 10A shows the consensus cumulative distribution functions (CDFs) of the consensus matrix, indicating strong support for K=3 referred to in Example 5. FIG. 10B shows the relative increase in the area under the CDFs, again indicating strong support for K=3. FIG. 10C shows a very clean consensus matrix for K=3, indicating that the Australian cohort also resulted in three subtypes.

FIG. 11 shows a table of the genes up-regulated in the invasive gastric cancer subtype.

FIG. 12 shows a table of the genes up-regulated in the proliferative gastric cancer subtype.

FIG. 13 shows a table of the genes up-regulated in the metabolic gastric cancer subtype.

FIG. 14 shows a table of the hypermethylated CpG sites of the invasive subtype.

FIG. 15 shows a table of the hypomethylated CpG sites of the invasive subtype.

FIG. 16 shows a table of the hypermethylated CpG sites of the proliferative subtype.

FIG. 17 shows a table of the hypomethylated CpG sites of the proliferative subtype.

FIG. 18 shows a table of the differentially expressed genes of the invasive subtype versus the proliferative subtype.

FIG. 19 shows a table of the differentially expressed genes of the invasive subtype versus the metabolic subtype.

FIG. 20 shows a table of the differentially expressed genes of the proliferative subtype versus the metabolic subtype.

### EXAMPLES

Non-limiting examples of the invention and a comparative example will be further described in greater detail by reference to specific Examples, which should not be construed as in any way limiting the scope of the invention.

### Example 1

### Ascertaining the Number of Gastric Cancer Subtypes

56 gastric adenocarcinomas were profiled and were combined with previously reported expression profiles to assemble a collection of 248 profiles (Gene Expression Omnibus Accession Nos. GSE15459 and GSE22183). All samples were profiled on Affymetrix U133 Plus 2.0 expression arrays.

The gene-expression microarray data assembled from multiple sources had obvious batch effects. Thus, to ensure that batch effects or non-biological variation between groups of samples arising from subtle but systematic differences in experimental procedures do not obscure biological structure or lead to artifactual findings, the data was processed by ComBat to remove the batch effects. The new merged dataset was termed “SG248”.

Assessment of whether biological substructure was preserved after processing by ComBat is as follows. First, consensus clustering was applied separately to each of the two individual batches, Singapore Cohort Batch A and Singapore Cohort Batch B. Second, the subsets of SG248 corresponding to the two batches were extracted and consensus clustering was applied to these two post-ComBat batches. The pre- and post-ComBat clusterings were then compared. All samples were assigned to the same clusters before and after ComBat processing as shown in Table 4 below.

TABLE 4

| pre-ComBat | | |
|---|---|---|
| | Cluster | Cluster |
| Cluster I | II | III |

Singapore Cohort, Batch A

| | | | | |
|---|---|---|---|---|
| post-ComBat | Cluster I | 60 | 0 | 0 |
| | Cluster II | 0 | 77 | 0 |
| | Cluster III | 0 | 0 | 55 |

Singapore Cohort, Batch B

| | | | | |
|---|---|---|---|---|
| post-ComBat | Cluster I | 13 | 0 | 0 |
| | Cluster II | 0 | 34 | 0 |
| | Cluster III | 0 | 0 | 9 |

Thus, it can be concluded that the biological substructure is preserved after ComBat processing.

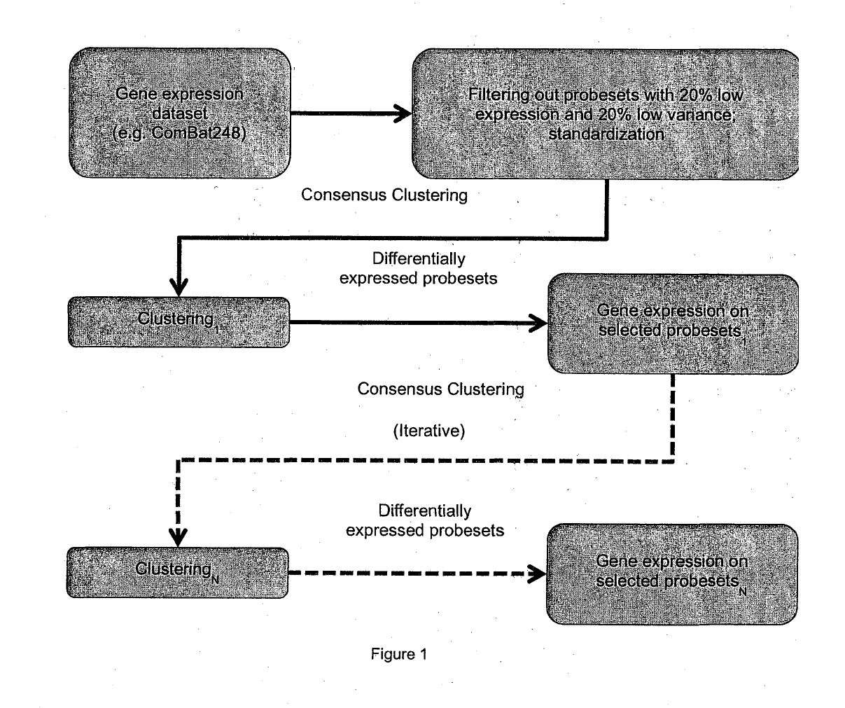

To discover natural subgroups of the SG248 tumors, an unsupervised clustering approach, consensus clustering in combination with iterative feature (i.e. probe set) selection was used. FIG. 1 describes the process involved in consensus clustering in combination with iterative feature selection.

Briefly, consensus clustering uses a resampling-based approach to assess confidence in the number of clusters and to assess confidence in the assignment of each sample to one of the clusters. Consensus clustering does this by repeatedly applying hierarchical clustering with average linkage over random subsets of 80% of the tumor samples. That is, probe sets with, median expression levels of less than 20th percentile of medians or with variance across all samples of less than the 20th percentile of variances are removed. The expression data was then standardized on probe sets and then arrays.

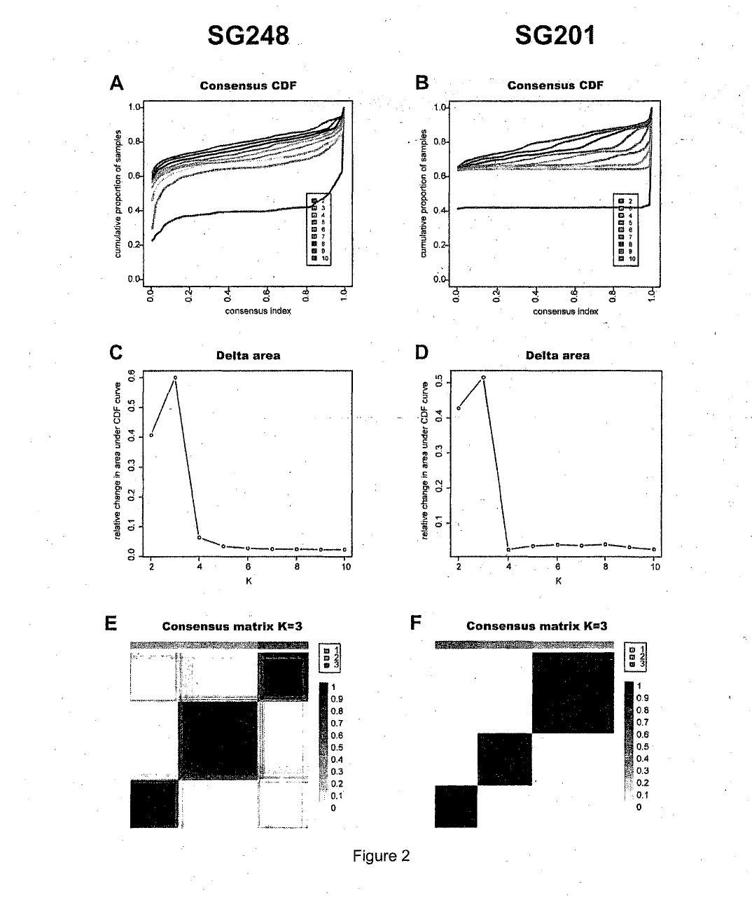

For every pair of samples, the proportion of resampling replicates in which the two samples are in the same cluster is their “consensus index”, and all the consensus indices together constitute the consensus-matrix (FIG. 2E). In an ideal consensus matrix, all consensus indices would be 1 or 0, indicating that each pair of samples always or never, respectively, clusters together.

Referring to FIG. 1, at each iteration, consensus clustering (R bioconductor package ConsensusClusterPlus) was carried out to derive the class assignment for each sample. Limma (R bioconductor package limma) was used to obtain the top 10,000 differentially expressed probe sets. In the next iteration, these selected probe sets were clustered and limma was applied to obtain the top 10,000 differentially expressed probe sets from all probe sets. This process was repeated until the class assignments of samples remained the same in two consecutive iterations. The procedure converged after three iterations. The rationale for iterative feature selection is that it improved clustering stability.

An important objective is to determine the number of clusters that are well supported by the data. For this, a standard approach that uses the “consensus cumulative distribution function” (CDF) was relied-upon. This is simply the cumulative distribution function of the consensus indices (FIG. 2A). As the number of clusters increases, the area under the CDF increases appreciably if a larger proportion of sample pairs have consensus indices near zero, indicating that they are well-separated by the clustering. At a certain point, however, increasing the number clusters does not appreciably increase the area under the CDF. This is either because some of the new clusters have very few members or because membership in some of the new clusters is unstable, resulting in consensus indices intermediate between 0 and 1.

Referring to FIG. 2A, the CDF plot shows that once the number of clusters, K, reaches three in this dataset, further increases do not yield appreciable increases in the area under the CDF. FIG. 2C shows the relative increase in the area under the CDF curves as K increases. This plot shows again that increasing the number of clusters beyond three yields little increase in the area under the CDF. Thus, there is strong support for the presence of three clusters of samples in this dataset. The three subtypes “invasive”, “proliferative”, and “metabolic” were termed based on the gene transcripts that are higher in each of the subtypes.

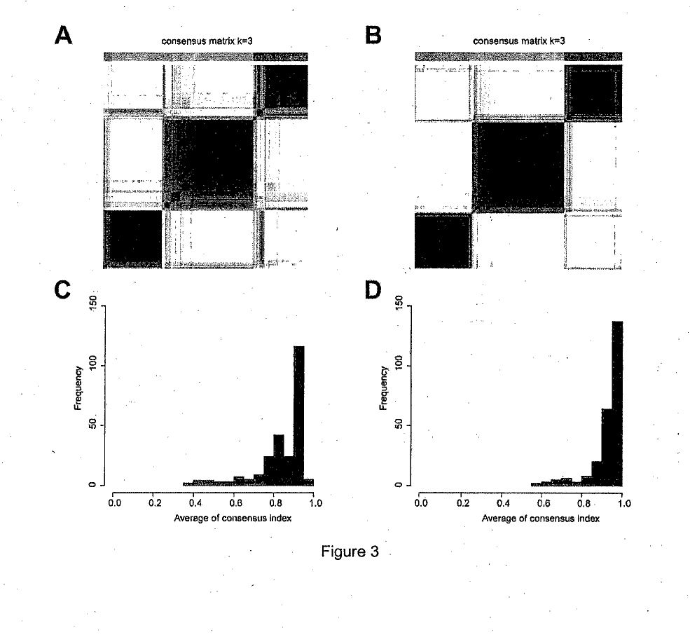

In addition, it was found that iterative selection of features (probe sets) substantially improved the separation of clusters, as seen in the consensus matrices constructed with and without iterative feature selection (FIG. 3).

In FIGS. 3, (A) and (C) show the consensus matrix (cophenetic correlation coefficient=0.958) and distribution of average consensus index without iterative feature selection. (B) and (D) show the consensus matrix (cophenetic correlation coefficient=0.987) and distribution of average consensus index with iterative feature selection. The average consensus index of a sample is defined as the average of its consensus indices vis-á-vis samples in the same cluster (i.e. in the same dark block in the consensus matrix). For a sample with completely stable cluster assignments, the average consensus index is 1.

Even with iterative feature selection, however, a proportion of samples may still have ambiguous cluster assignments, as indicated by consensus indices intermediate between 0 and 1. These samples were identified as follows: the average consensus index of a sample was defined as the average of its consensus indices vis-à-vis samples in the same cluster (i.e. in the same dark block in the consensus matrix in FIG. 2E). For a sample with completely stable cluster assignments, the average consensus index is 1. The 201 samples with average consensus indices >0.9 was considered as representative of their clusters and constructed a new dataset, termed “SG201”, comprised of these samples. Consensus clustering with iterative feature selection on SG201 yields a nearly perfect clustering (FIG. 2B, D, F), with cophenetic correlation coefficient=1. Consensus clustering with iterative feature selection on SG201 was also performed using the K-means clustering algorithm with K=3.

Concordance with the initial clustering was 99.5%, demonstrating strong support for the three subtypes.

Further analysis was based on SG201.

### Example 2

### Genomic Copy-Number Alterations (CNA)

138 of the SG201 tumor samples were analyzed using Affymetrix SNP6 DNA microarrays (Affymetrix, 2009) and 98 non-malignant samples as a reference. Using these data, the number of cytobands affected by CNA for each tumor was determined.

Preprocessing and normalization were performed using Affymetrix Genotyping Console 3.0 (Affymertrix, 2008). For each tumor sample, “Log 2Ratios” were generated (terminology from Affymetrix Genotyping Console) for the SNP (single nucleotide polymorphism) and copy-number probe sets on the array. Circular binary segmentation was applied to the Log 2Ratios using the R package DNAcopy (http://www.bioconductor.org/packages/2.3/bioc/html/DNAcopy.html).

The segmented data was then mapped to cytobands using hg18 cytoband positions from the UCSC Genome Browser database. The copy number of each cytoband was estimated as the length-weighted average of the Log 2Ratios of the segments within the cytoband. Our thresholds for calling a copy number gain or loss for a cytoband were >0.2 or <−0.2.

For the nonnegative-matrix-factorization-based clustering of tumor samples by their cytoband CNA levels, the published Matlab code was used.

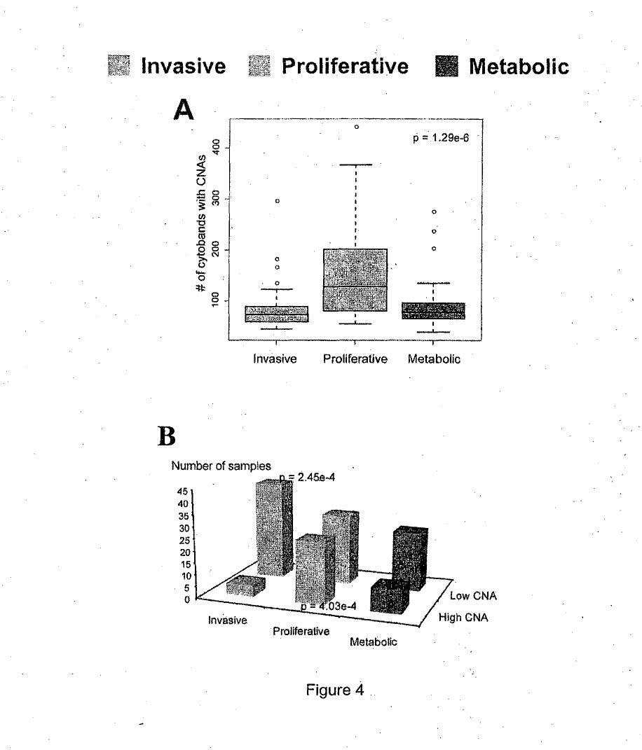

The genomes of proliferative tumors were found to have significantly more CNA than the other two subtypes (FIG. 4A). We then used nonnegative matrix factorization to cluster the samples by the degree of CNA of each cytoband. This revealed two groups, one of which has both more cytobands affected by CNA and more extreme CNAs.

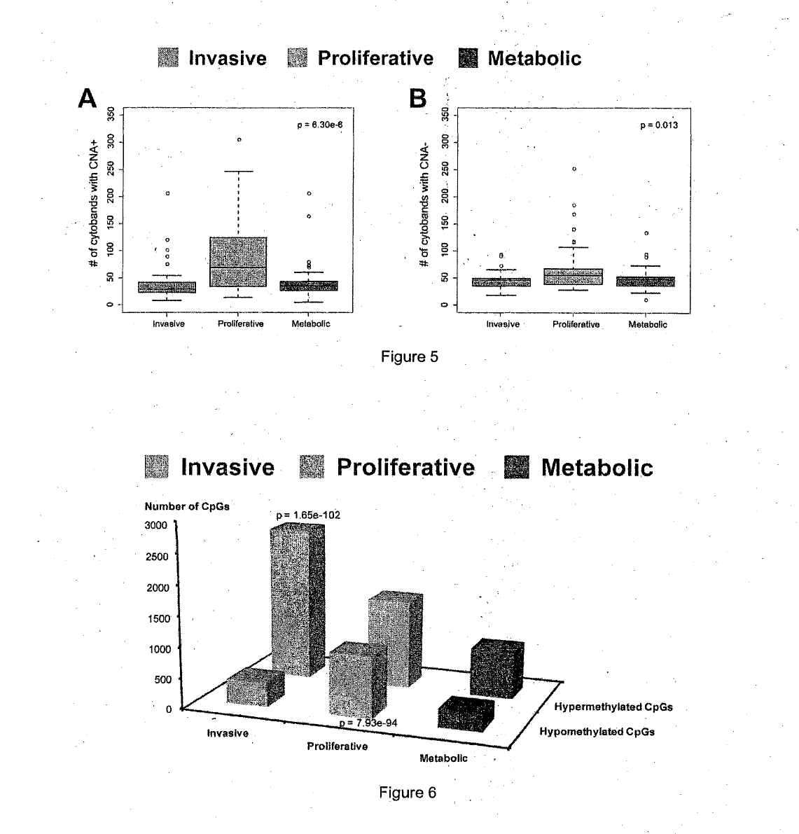

Specifically, invasive-subtype tumors are significantly enriched for low-CNA tumors (p=2.45×10-4, hypergeometric test) and proliferative-subtype tumors are significantly enriched for high-CNA tumors (p=4.03×10-4, hypergeometric test) (FIG. 4B). Enrichment for high-CNA tumors in the proliferative subtype is primarily due to copy number gains (FIG. 5).

In FIG. 5, (A) shows the distribution of the number of cytobands with copy number gain in each subtype and corresponding boxplot for three subtypes. Kruskal-Wallis test shows there is a significant difference of median value among these groups (p=6.30e-6). (B) shows the distribution of the number of cytobands with copy number loss in each subtype and corresponding boxplot for three subtypes. Kruskal-Wallis test shows there is a much less significant difference of median value among these groups (p=0.013).

Together this shows that CNA gain is more significantly enriched than CNA loss in the proliferative subtype.

The tumor samples were also examined for CNAs affecting specific oncogenes. For analysis of copy number alterations of the oncogenes, the segmented Log 2Ratios was mapped to the genes' genomic regions. It was found that the proliferative subtype is enriched for genomic amplifications of MYC, KRAS and HER2 as shown in Table 5 below.

TABLE 5

| | | | | p-value |
|---|---|---|---|---|
| | Subtype | | | (Fisher's |
| Gene | Invasive (47) | Proliferative (56) | Metabolic (35) | exact test) |
| MYC | 2 | 19 | 3 | 1.04E−04 |
| ERBB2 | 2 | 9 | 0 | 0.009 |
| KRAS | 1 | 9 | 2 | 0.040 |
| EGFR | 3 | 10 | 4 | 0.205 |
| FGFR2 | 7 | 7 | 2 | 0.443 |

### Example 3

### Methylation Profiles

DNA methylation profiles were determined for 139 of the SG201 tumor samples. Illumina Infinium HumanMethylation27 arrays (Weisenberger et al., 2008) was used to assess methylation levels at 26,486 autosomal CpGs across the genome, including 94 non-malignant samples from the Singapore cohort as reference.

The extent to which the DNA-methylome of each subtype differs from the 94 nonmalignant gastric tissue samples was investigated. To do this, methylation levels of each CpG across all tumors in each subtype were compared to methylation levels in the non-malignant samples. DNA methylation levels (P value) for each probe were computed using Illumina's Genome Studio software. Differential methylation analyses were performed on the invasive-subtype, proliferative-subtype, and metabolic-subtype sample sets separately relative to 94 non-malignant samples. CpGs with significantly differential methylation levels between two groups (for example, invasive-subtype tumors compared to 94 non-malignant samples) were identified by two sided t-tests with a Bonferroni corrected alpha of 0.05/26,486.

CpG sites in which the tumor samples of in a subtype were significantly hyper- or hypomethylated compared to the non-malignant samples were considered to be aberrantly methylated.

The invasive subtype showed the largest number of aberrantly methylated sites, with 2,928 (11.1%) of the assayed CpGs showing methylation that is significantly different from that in non-malignant tissues. In the proliferative subtype, 2,462 (9.3%) of the CpGs show significant differences from non-malignant tissues. In the metabolic subtype, only 1,079 (4.1%) of the CpGs show significant differences from non-malignant tissues.

The three subtypes differ greatly with respect to whether aberrantly methylated CpGs are hyper- and hypomethylated relative to non-malignant samples. As shown in FIG. 6, invasive-subtype tumors are significantly enriched for hypermethylated sites (p=1.65×10-102, hypergeometric test), and proliferative-subtype tumors are significantly enriched for hypomethylated sites (p=7.93×10-94, hypergeometric test), and metabolic-subtype tumors are enriched for neither.

### Example 4

A study was done to determine whether there was a difference in survival among the three subtypes. In this study, cancer-specific death was considered as an event unless stated otherwise.

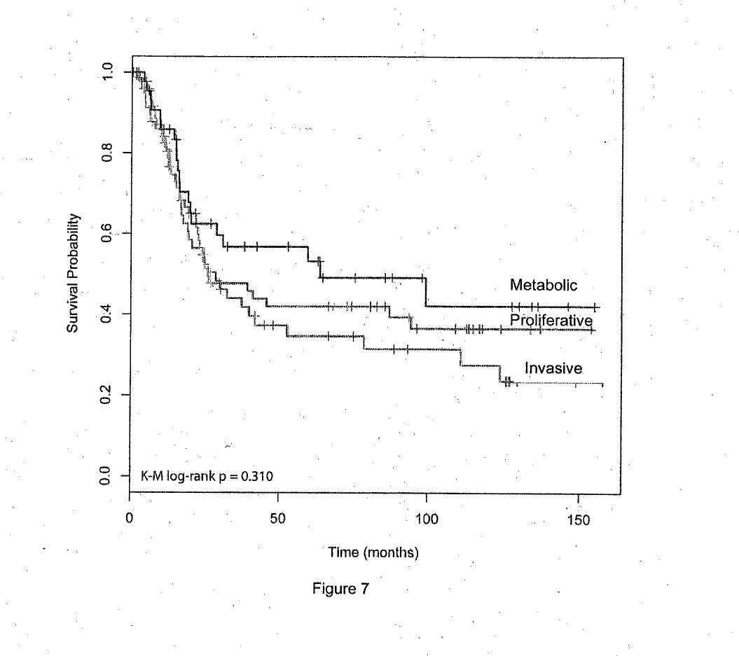

Data from 188 patients in the SG201 Singapore cohort for which survival information was available was analyzed. As shown in FIG. 7, Kaplan-Meier analysis based on Log-Rank test with p=0.310 indicates no significant survival difference among the three subtypes, although there is a trend for invasive-subtype patients to have worse survival.

Among univariate Cox proportional hazards models, TNM stage and grade are the only factors associated with survival as shown in Table 6 below.

TABLE 6
Univariate Cox Regression analysis in Singapore Cohort

| Covariate | | Hazard ratio (95% CI) | p-value |
|---|---|---|---|
| Subtype | Metabolic | 1 | |
| | Proliferative | 1.279 (0.744, 2.199) | 0.374 |
| | Invasive | 1.528 (0.884, 2.640) | 0.129 |
| Stage | I | 1 | |
| | II | 3.448 (1.112, 10.70) | 0.032 |
| | III | 11.159 (3.965, 31.40) | 4.89e−6 |
| | IV | 20.294 (7.086, 58.12) | 2.05e−8 |
| Treatment | Surgery | 1 | |
| | alone | | |
| | 5-FU | 1.656 (0.983, 2.788) | 0.058 |
| Age | Numeric | 0.992 (0.975, 1.009) | 0.369 |
| Gender | Female | 1 | |
| | Male | 1.213 (0.789, 1.864) | 0.379 |
| Lauren | Mixed | 1 | |
| | Intestinal | 1.150 (0.520, 2.546) | 0.730 |
| | Mixed | 1.715 (0.765, 3.848) | 0.191 |
| Grade | Low | 1 | |
| | High | 1.614 (1.046, 2.489) | 0.031 |
| Resection | Negative | 1 | |
| Margins | Positive | 1.654 (0.936, 2.925) | 0.084 |
| Tumor site | Upper | 1 | |
| | Middle | 1.129 (0.526, 2.420) | 0.756 |
| | Lower | 1.775 (0.818, 3.853) | 0.147 |
| H. Pylori | No | 1 | |
| | Yes | 1.020 (0.562, 1.851) | 0.948 |

Further analysis with a multivariate Cox proportional hazards regression, adjusted for stage and grade, likewise detects no significant differences among the three subtypes in term of the hazard ratios. Table 7 below shows the results.

TABLE 7
Multivariate Cox Regression analysis in Singapore Cohort

| Covariate | | Hazard ratio (95% CI) | p-value |
|---|---|---|---|
| Subtype | Metabolic | 1 | |
| | Proliferative | 1.476 (0.844, 2.582) | 0.173 |
| | Invasive | 1.506 (0.857, 2.644) | 0.154 |
| Stage | I | 1 | |
| | II | 3.428 (1.101, 10.668) | 0.034 |
| | III | 10.590 (3.732, 30.049) | 9.22e−6 |
| | IV | 19.876 (6.864, 57.556) | 3.57e−8 |
| Grade | Low | 1 | |
| | High | 1.209 (0.775, 1.885) | 0.404 |

Many of the patients in the Singapore cohort were treated with 5-FU. To investigate the effect of 5-FU treatment on patient survival, the survival of Singapore and Australian patients treated with surgery alone were compared to that of patients treated by surgery and adjuvant 5-FU based therapy.

The clinical decision on administration of 5-FU was based on multiple factors, including the patient's general health, risk of relapse (estimated largely by disease stage), treatment-related toxicities, and patient preference.

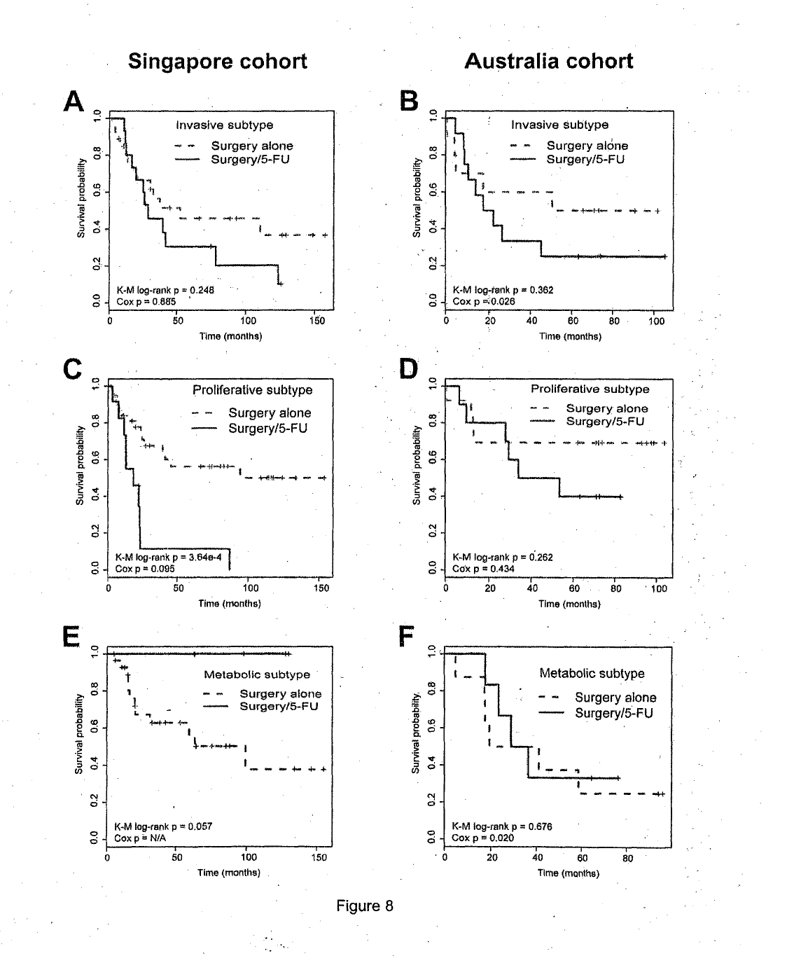

FIGS. 8A to F show Kaplan-Meier plots for surgery plus 5-FU versus surgery alone for each of the three subtypes. In patients with invasive and proliferative subtypes, survival is worse among those treated with 5-FU. This is mainly because patients with higher TNM-stages were more likely to receive 5-FU in addition to surgery. Multivariate Cox regression does not suggest any benefit from 5-FU treatment of patients in these two subtypes (see Table 8 below).

TABLE 8

| | | |
|---|---|---|
| Covariate | HR (95% CI) | p-value |

Singapore Cohort, Invasive Subtype

| | | | |
|---|---|---|---|
| Treatment | Surgery alone | 1 | |
| | 5-FU | 1.062 (0.469, 2.407) | 0.885 |
| Stage | I | 1 | |
| | II | 4.799 (0.545, 42.294) | 0.158 |
| | III | 17.460 (2.149, 141.874) | 7.46e−3 |
| | IV | 36.870 (3.915, 347.224) | 1.62e−3 |

Singapore Cohort, Proliferative Subtype

| | | | |
|---|---|---|---|
| Treatment | Surgery alone | 1 | |
| | 5-FU | 2.105 (0.879, 5.043) | 0.095 |
| Stage | I | 1 | |
| | II | 0.851 (0.053, 13.664) | 0.909 |
| | III | 8.028 (1.032, 62.431) | 0.047 |
| | IV | 26.062 (2.984, 227.603) | 2.19e−3 |

Singapore Cohort, Metabolic Subtype
(As there were no deaths among 5-FU treated patients, Cox
regression is not available)
Australian Cohort, Invasive Subtype

| | | | |
|---|---|---|---|
| Treatment | Surgery alone | 1 | |
| | 5-FU | 0.222 (0.059, 0.833) | 0.026 |
| Stage | I/II | 1 | |
| | III/IV | 70.736 (6.565, 762.188) | 4.46e−4 |

Australian Cohort, Proliferative Subtype

| | | | |
|---|---|---|---|
| Treatment | Surgery alone | 1 | |
| | 5-FU | 1.938 (0.369, 10.170) | 0.434 |
| Stage | I/II | 1 | |
| | III/IV | 1.080 (0.206, 5.667) | 0.927 |

Australian Cohort, Metabolic Subtype

| | | | |
|---|---|---|---|
| Treatment | Surgery alone | 1 | |
| | 5-FU | 0.070 (7.50e−3, 0.659) | 0.020 |
| Stage | I/II | 1 | |
| | III/IV | 52.576 (3.859, 716.221) | 2.94e−3 |

NOTE:
HR = hazard ratio

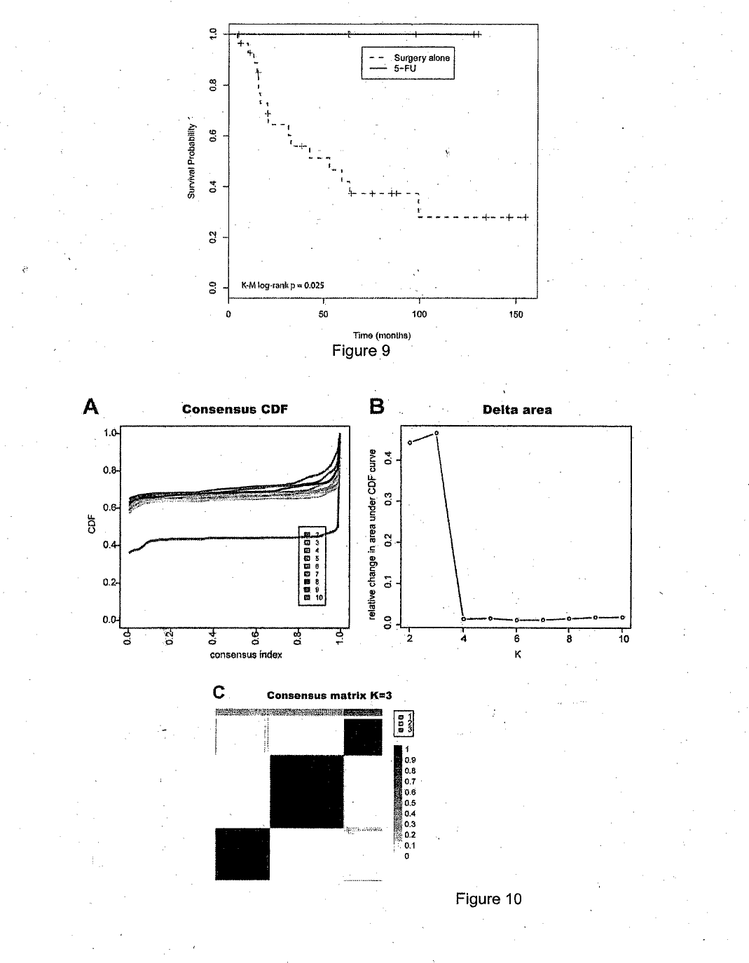

However, among metabolic-subtype patients, survival is better for those treated with 5-FU, even though patients treated with 5-FU had higher TNM stages. When adjusted for stage, patients with the metabolic subtype and treated with 5-FU had a hazard ratio of 0.070 (95% CI 7.50×10-3 to 0.659, p=0.020). Because there were no deaths among 5-FU-treated metabolic-subtype patients, a Cox proportional hazards regression is not possible. However, as shown in FIG. 9, a Kaplan-Meier analysis with disease-free survival as the endpoint shows that metabolic-subtype patients benefited significantly from 5-FU treatment compared to surgery alone (p=0.025 log, rank test).

Finally, in a combination analysis of 197 patients from the Singapore and Australian cohorts, a significant interaction between the metabolic-subtype and 5-FU treatment was observed (see Table 9 below).

TABLE 9
197 patients in SG and AU cohorts (SG201T + AU70T)
Model: ~subtype * treatment + stage

| Covariate | | Hazard Ratio (95% CI) | p-value |
|---|---|---|---|
| Subtype | Proliferative | 1 | |
| | Invasive | 1.617 (0.840, 3.113) | 0.151 |
| | Metabolic | 1.362 (0.709, 2.617) | 0.353 |
| Treatment | Surgery alone | 1 | |
| | 5-FU | 1.437 (0.728, 2.835) | 0.296 |
| Stage | I | 1 | |
| | II | 3.121 (1.130, 8.624) | 0.028 |
| | III | 9.974 (3.882, 25.621) | 1.77e−6 |
| | IV | 20.827 (7.582, 57.208) | 3.87e−9 |
| Subtype: treatment | Proliferative: 5- | 1 | |
| interaction | FU | | |
| | Invasive: 5-FU | 0.722 (0.287, 1.817) | 0.489 |
| | Metabolic: 5-FU | 0.196 (0.054, 0.711) | 0.013 |

Accordingly, further evidence is provided that metabolic-subtype patients benefited from 5-FU therapy.

### Example 5

An ensemble of three NTP predictors, termed “GCPred” was used to classify new samples into one of the three subtypes. Each constituent predictor was built with genes that were differentially expressed between one pair of subtypes. Limma was used to obtain the top differentially expressed probe sets between two subtypes, using FDR <0.001 and absolute fold change >1.5 as thresholds. The gene signatures and t-scores then served as the features and weights in the constituent NTPs. The GCPred ensemble determines the subtype of a sample as follows: If two of the three constituent predictors make the same classification, with at least one FDR <0.05, then GCPred uses that classification. If neither FDR is <0.05 or if all three constituent NTP classifications are different, then GCPred does not consider the test sample to be classifiable.

To assess the accuracy of GCPred, five-fold cross validation was used. The SG201 dataset was split into five subsets of roughly equal size. Each of the subsets was considered a test set and an NTP ensemble predictor was trained on the remaining data and used to classify the test set. 199 out of 201 samples were classified, and of these, 193 were correct, corresponding to an overall accuracy of 97%.

In addition, the performance of the GCPred in a second independent cohort of 70 primary gastric tumors from Australian patients was assessed (see Supplementary Table 10 below).

TABLE 10

| | Singapore | Singapore | Australia |
|---|---|---|---|
| | (n = 248) | (n = 201) | (n = 70) |
| Lauren Classification | | | |
| Diffuse | 86 | 66 | 30 |
| Intestinal | 138 | 115 | 34 |
| Mixed | 22 | 18 | 6 |
| Tumor Grade | | | |
| Low | 94 | 77 | 24 |
| High | 149 | 120 | 46 |
| Age | 64.79 ± 12.82 | 64.50 ± 13.01 | 65.54 ± 12.53 |
| Tumor site | | | |
| Upper | 30 | 25 | — |
| Middle | 104 | 82 | — |
| Lower | 62 | 49 | — |
| Gender | | | |
| Female | 87 | 69 | 22 |
| Male | 159 | 131 | 48 |
| Recurrence | | | |
| Yes | 103 | 82 | 40 |
| No | 136 | 111 | 30 |
| Stage | | | |
| 1 | 42 | 34 | 13 |
| 2 | 40 | 34 | 16 |
| 3 | 90 | 71 | 33 |
| 4 | 73 | 60 | 7 |
| H. Pylori status | | | |
| Yes | 82 | 62 | — |
| No | 58 | 51 | — |
| G-INT/G-DIF | | | |
| G-INT | 143 | 116 | 38 |
| G-DIF | 105 | 85 | 32 |
| Median follow-up | 19.60 | 20.02 | 31.77 |
| interval (months) | | | |

First, GCPred was used to classify each these 70 samples. GCPred classified all 70 samples. Second, SG201 was co-clustered with the 70 Australian samples using ComBat. A new dataset called SG_AU_{—}271 resulted. Consensus clustering with iterative feature selection on SG_AU_{—}271 was then carried out and the results are shown in FIG. 10.

In FIG. 10, (A) shows the consensus cumulative distribution functions (CDFs) of the consensus matrix for K=2, 3, . . . , 10, indicating strong support for K=3. (B) shows the relative increase in the area under the CDFs for K=2, 3, . . . , 10, again indicating strong support for K=3. (C) shows a very clean consensus matrix for K=3 (cophenetic correlation coefficient=0.996).

Each Australian sample was classified according to the subtypes of the Singapore samples with which it co-clustered. The concordance between GCPred and co-clustering for the 70 Australian samples was 94.3%, demonstrating GCPred's reliability.

### Example 6

It was then examined whether there are differences in 5-FU sensitivity, in vitro, between gastric cancer cell lines assigned to the three subtypes.

GCPred was first used to predict the subtypes of 28 cell lines for which 5-FU GI50 values had previously been determined. Metabolic-subtype cells were significantly more sensitive to 5-FU (p=1.47×10-3) for metabolic versus proliferative using the Wilcoxon rank sum test.

In addition, GCPred was used to classify the NCI-60 cell lines into the three subtypes. It was observed that there is preferential sensitivity of metabolic-subtype NCI-60 cells to 5-FU (p=0.036) for metabolic versus invasive using the Wilcoxon rank sum test, suggesting that the metabolic-subtype classification and its sensitivity 5-FU are relevant to cancers in addition to gastric adenocarcinoma.

1. A grouping for classifying a gastric cancer tumor sample obtained from a patient suffering or suspected to suffer from gastric cancer, wherein the grouping comprises an invasive subtype, a proliferative subtype and a metabolic subtype,
wherein the invasive subtype is characterized by any one or more or all of the following:
(a) compared to the proliferative subtype and the metabolic subtype, the up-regulated genes in the invasive subtype are associated with any one of the following pathways: focal adhesion, extracellular-matrix-receptor interaction, gap junction, calcium signaling pathway, complement cascades, coagulation cascades, tight junction, regulation of actin cytoskeleton, cell adhesion, vasculature development, blood vessel development, regulation of cell motion, cell motility, extracellular matrix organization, cell-matrix adhesion, angiogenesis, response to wounding, wound healing, and BMP signaling pathway;
(b) compared to the proliferative subtype and the metabolic subtype, gene sets that have increased gene set activities in the invasive subtype are selected from the group consisting of: p53, EMT (epithelial-mesenchymal transition), TGF-β, VEGF, NFκB, mTOR, SHH (sonic hedgehog), and CSC (cancer stem cell);
(c) compared to the proliferative subtype and the metabolic subtype, invasive subtype tumors are significantly enriched with low-CNA (copy number alteration) tumors;
(d) compared to non-malignant tissues, the number of aberrantly methylated CpG sites in the invasive subtype is higher than those in the proliferative subtype and metabolic subtype;
(e) the number of aberrantly hypermethylated sites in the invasive subtype is higher than in the proliferative subtype and the metabolic subtype;
(f) invasive subtype tumors are not or almost not enriched for TP53 missense mutations compared to the proliferative subtype;
(g) the invasive subtype shows strong association to the ‘diffuse’ tumor type according to Lauren classification;
(h) compared to the proliferative subtype and the metabolic subtype, the cellular differentiation of invasive subtype tumors is undifferentiated or poorly differentiated;
(i) the invasive subtype is more sensitive to compounds targeting the PI3K/AKT/mTOR pathway than in the proliferative and the metabolic subtype; and
(j) the invasive subtype shows cancer-stem-cell-like properties;
wherein the proliferative subtype is characterized by any one or more or all of the following:
(a) compared to the invasive subtype and the metabolic subtype, the up-regulated genes in proliferative subtype are associated with any one of the following pathways: cell cycle pathway, nuclear division and cell division;
(b) compared to the invasive subtype and the metabolic subtype, gene sets that have increased gene set activities in the proliferative subtype are selected from the group consisting of: E2F, MYC, and RAS;
(c) compared to the invasive subtype and the metabolic subtype, proliferative subtype tumors are significantly enriched in high-CNA tumors;
(d) compared to the invasive subtype and the metabolic subtype, the proliferative subtype is enriched with genomic amplifications of CCNE1, MYC, KRAS, and ERBB2 (also known as HER2);
(e) the number of aberrantly hypomethylated CpG sites in the proliferative subtype is higher than in the invasive subtype and the metabolic subtype;
(f) proliferative subtype tumors are enriched with hypomethylated CpG sites compared to the invasive subtype and the metabolic subtype;
(g) proliferative subtype tumors are enriched with TP53 missense mutations compared to the invasive subtype and the metabolic subtype;
(h) the proliferative subtype shows strong association to the ‘intestinal’ tumor type according to Lauren classification; and
(i) compared to the invasive subtype and the metabolic subtype, the cellular differentiation of proliferative subtype tumors is well-differentiated or moderately-differentiated;
wherein the metabolic subtype is characterized by any one or more or all of the following:
(a) compared to the proliferative subtype and the invasive subtype, the up-regulated genes in metabolic subtype are associated with any one of the following pathways: metabolic processes, digestion and secretion;
(b) compared to the invasive subtype and the proliferative subtype, the gene set of spasmolytic polypeptide/(TFF2)-expressing-metaplasia (SPEM) in the metabolic subtype has increased gene set activity;
(c) metabolic subtype tumors are not or almost not enriched for TP53 missense mutations compared to the proliferative subtype;
(d) metabolic subtype tumors have significantly lower expression of both thymidylate synthase (TS) and dihydropyrimidine dehydrogenase (DPD) transcripts compared to the invasive subtype and the proliferative subtype; and
(e) the chance of survival of patients suffering from gastric cancer or suspected to suffer from gastric cancer is higher when treated with adjuvant 5-fluorouracil compared to when undergoing surgery alone.

2. The grouping of claim 1, wherein the grouping for classifying gastric cancer patients is based on the gene expression profile obtained from primary gastric tumors.

3. The grouping of claim 1, wherein the hypermethylation signature and hypomethylation signature are obtained by determining CpG sites of each subtype that were hypo- and hypermethylated in the respective subtype.

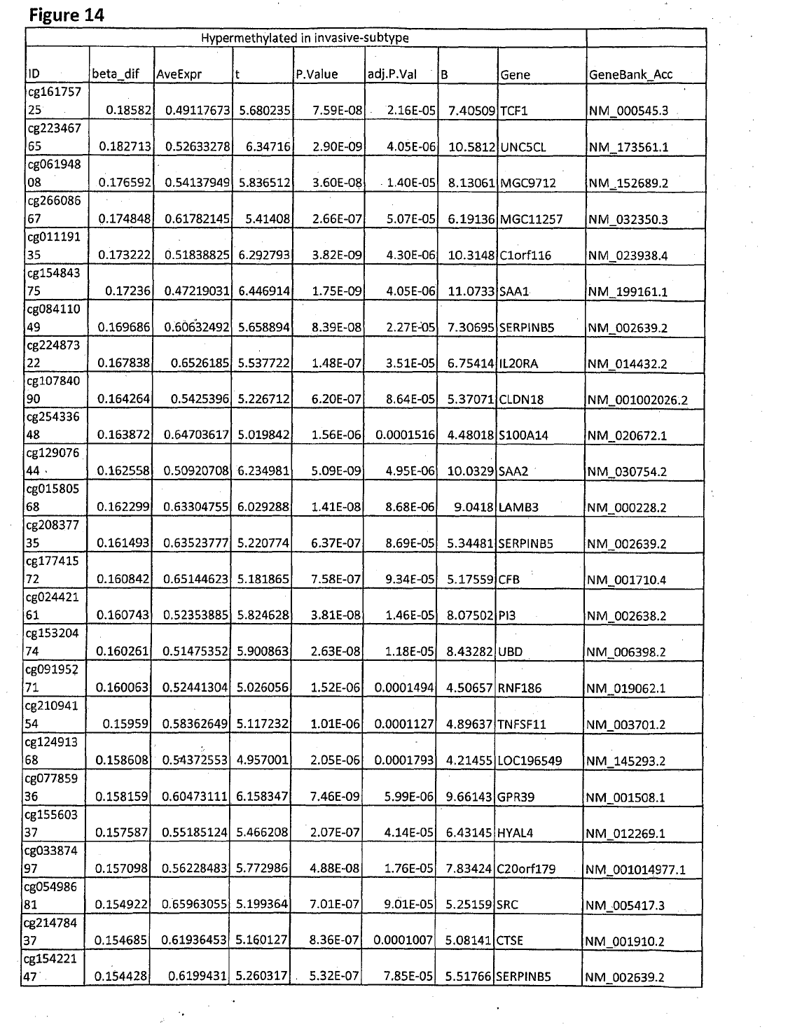

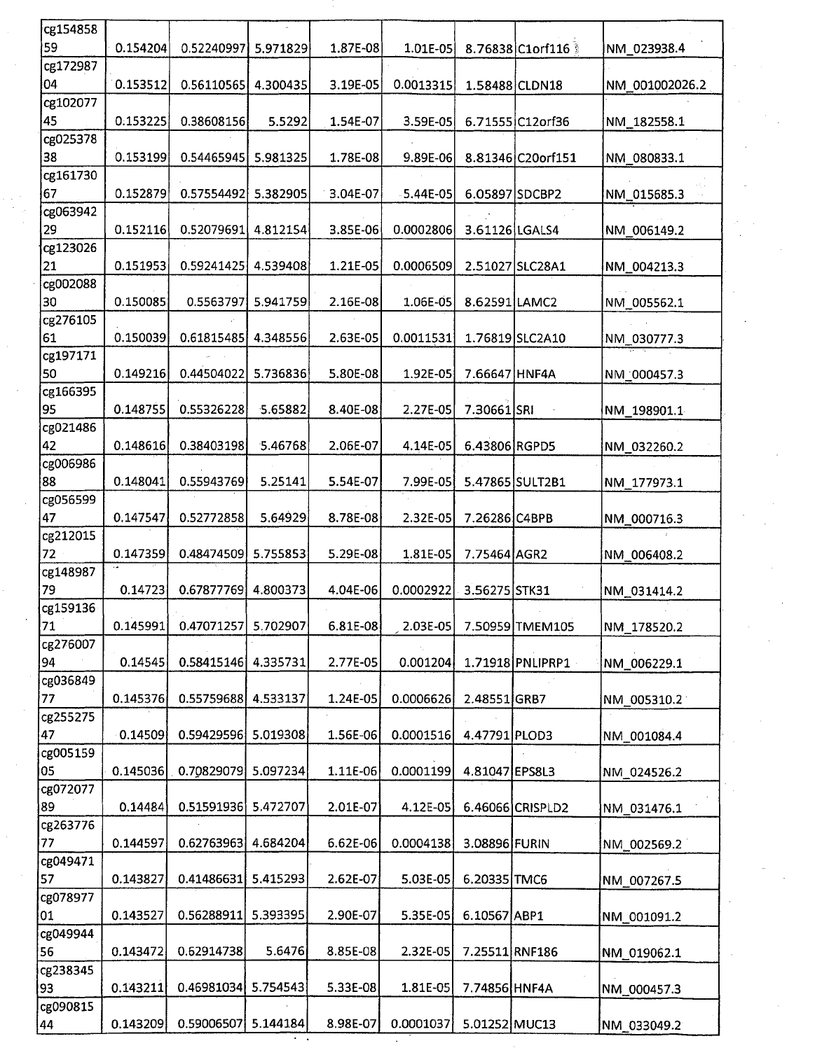

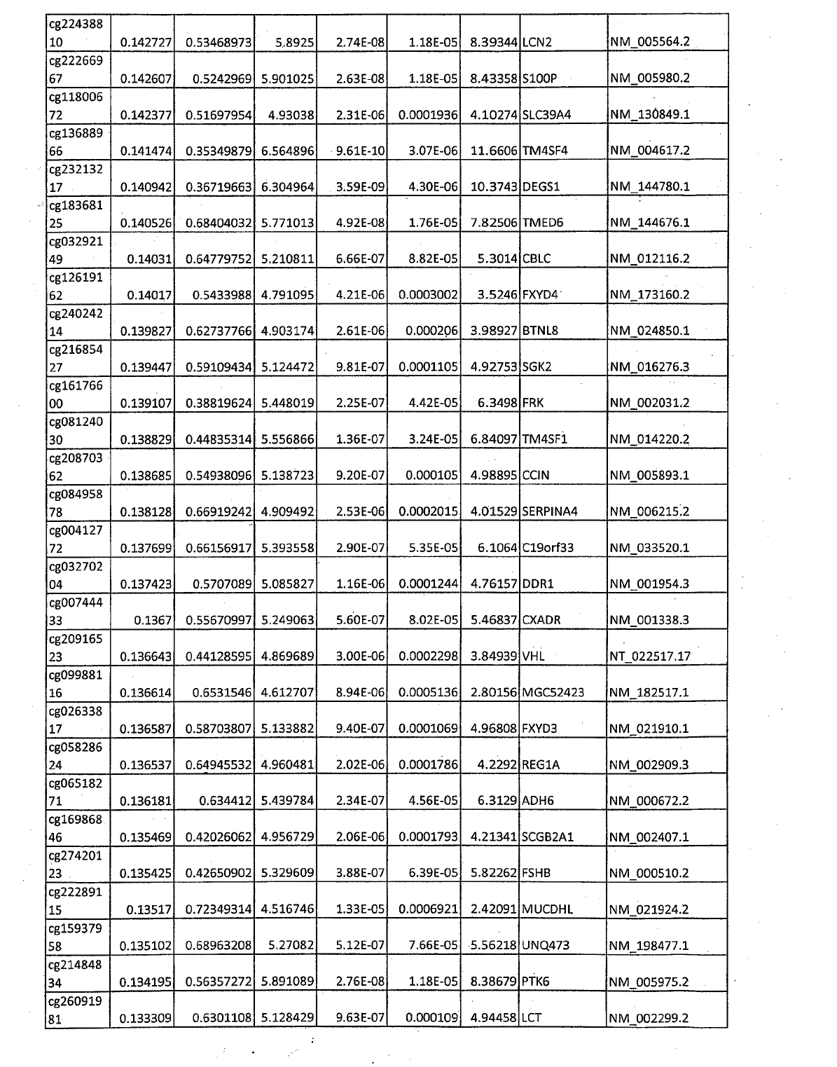

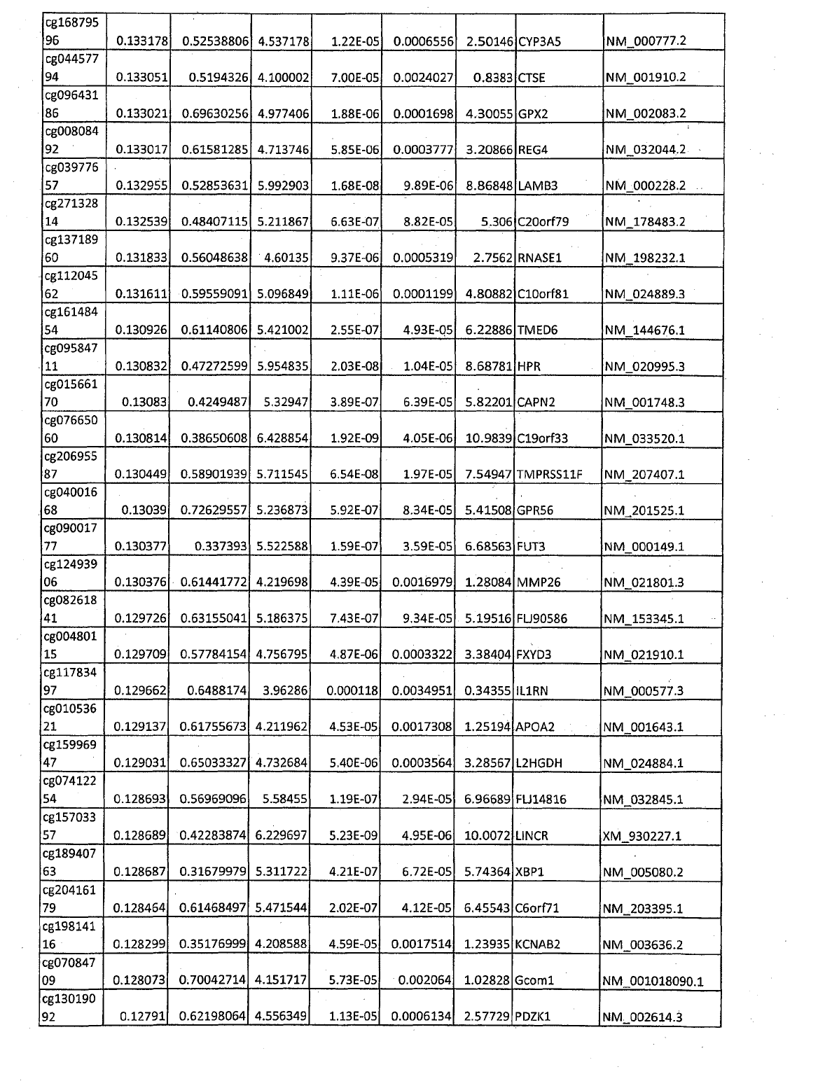

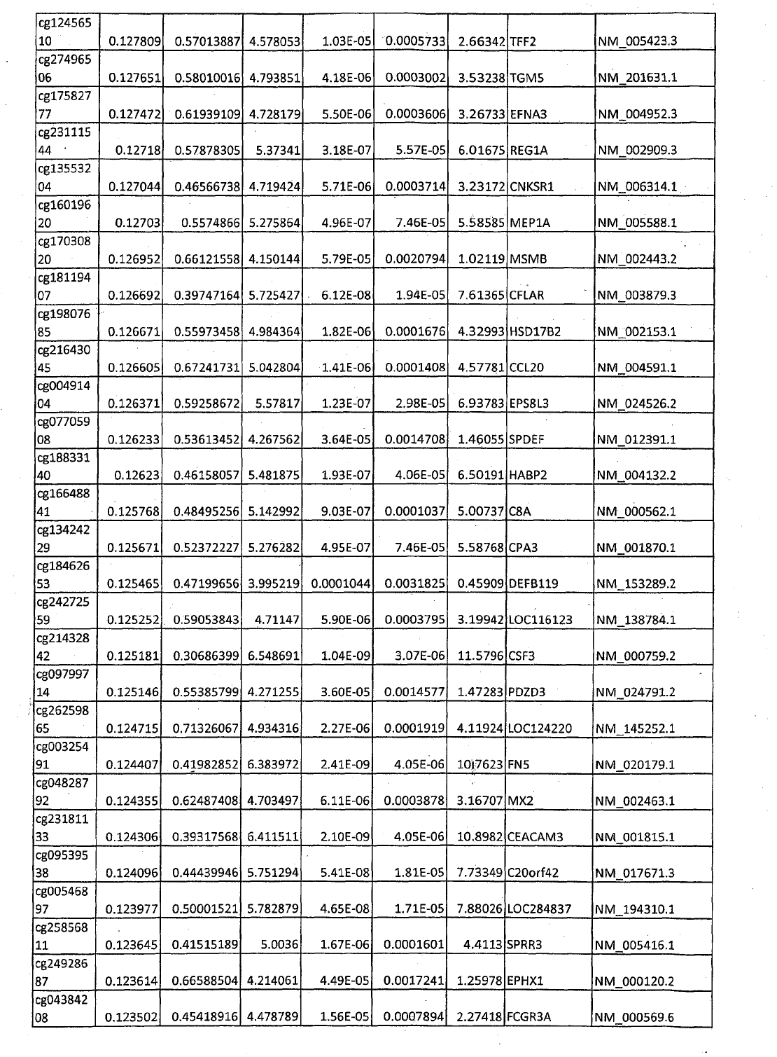

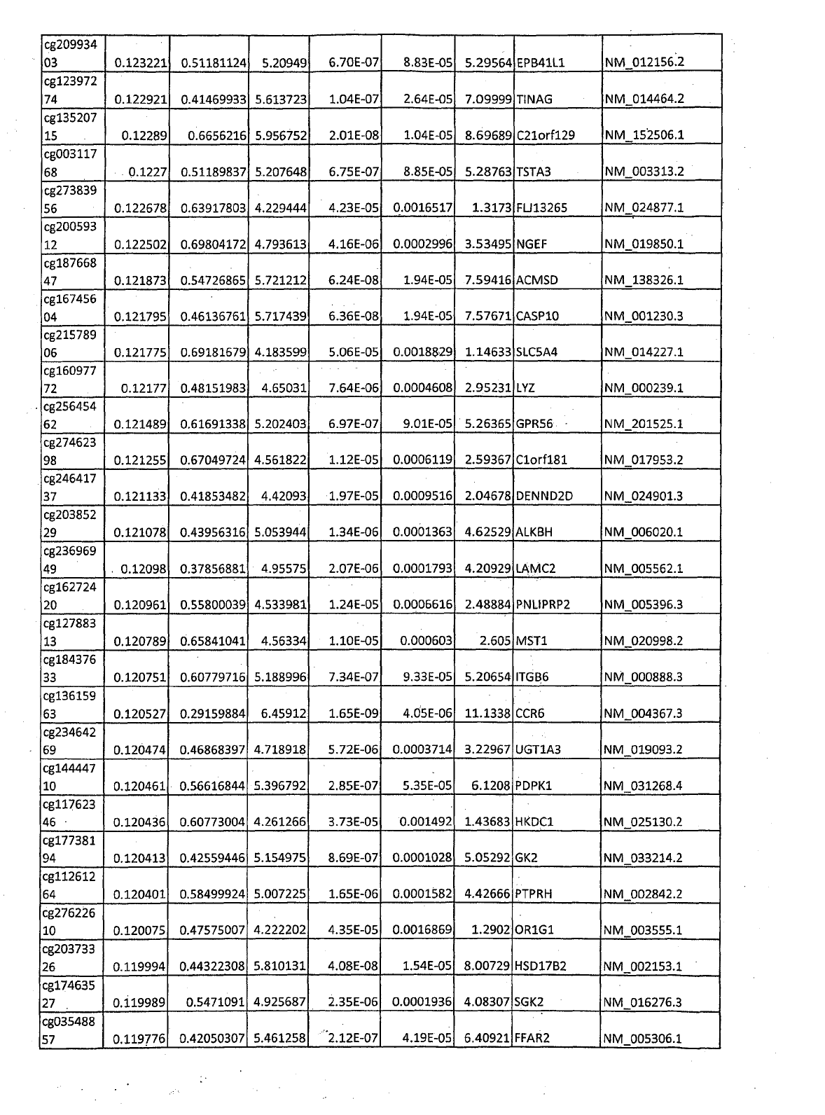

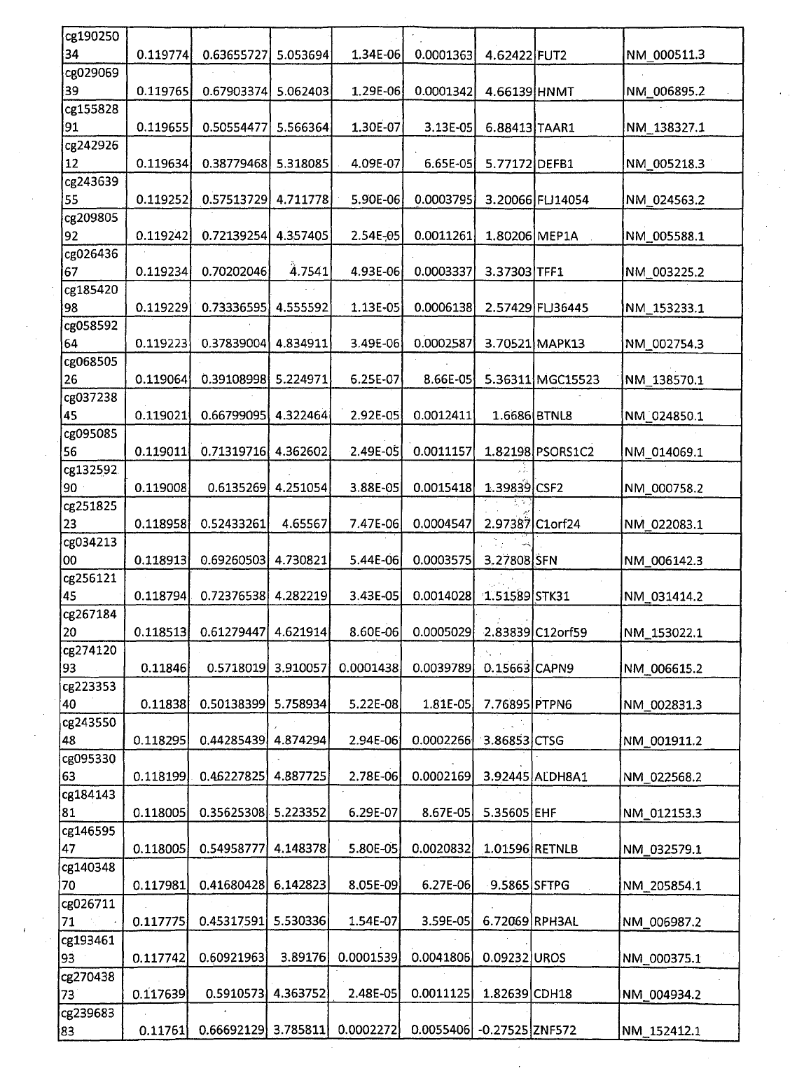

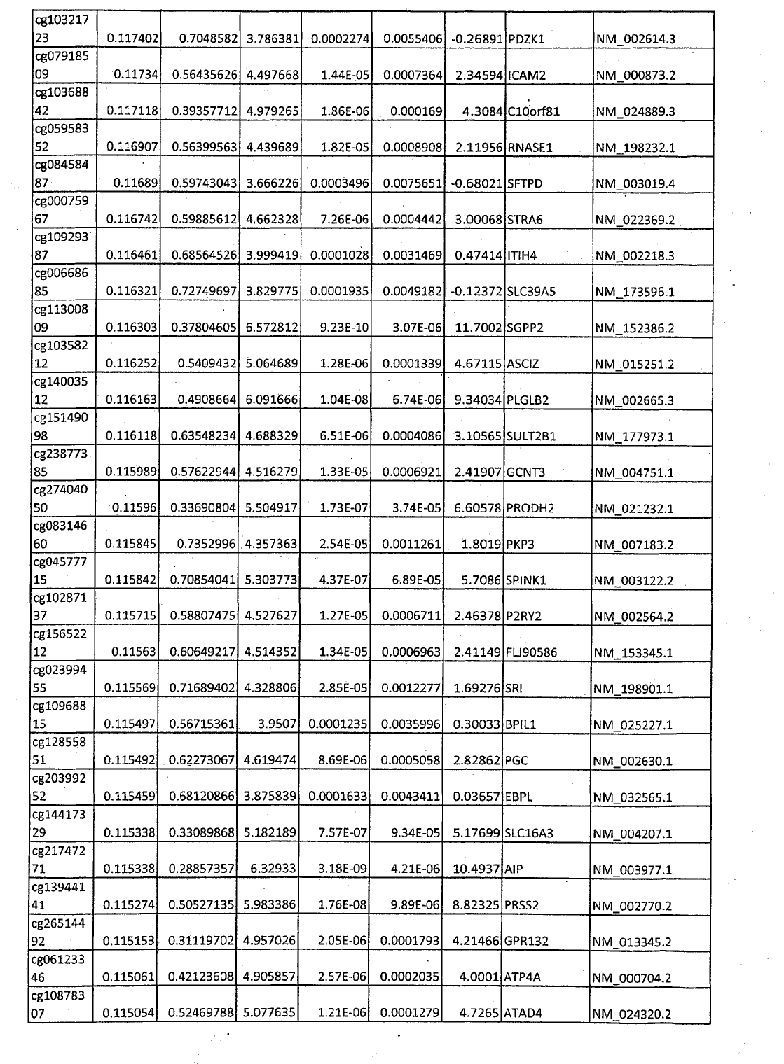

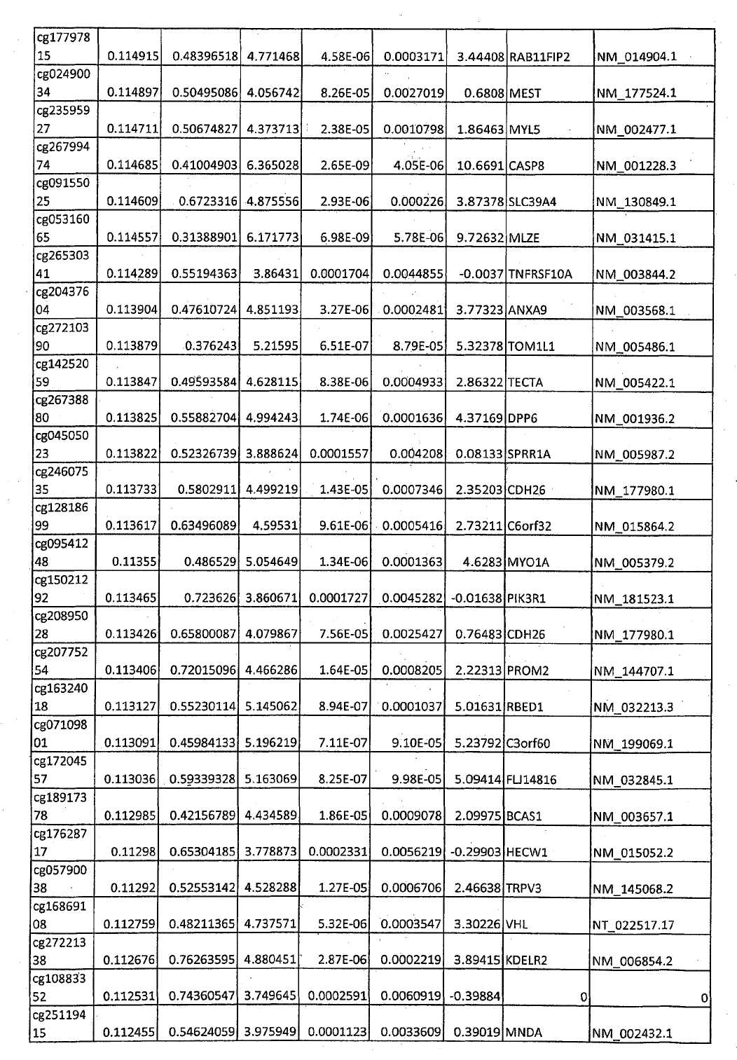

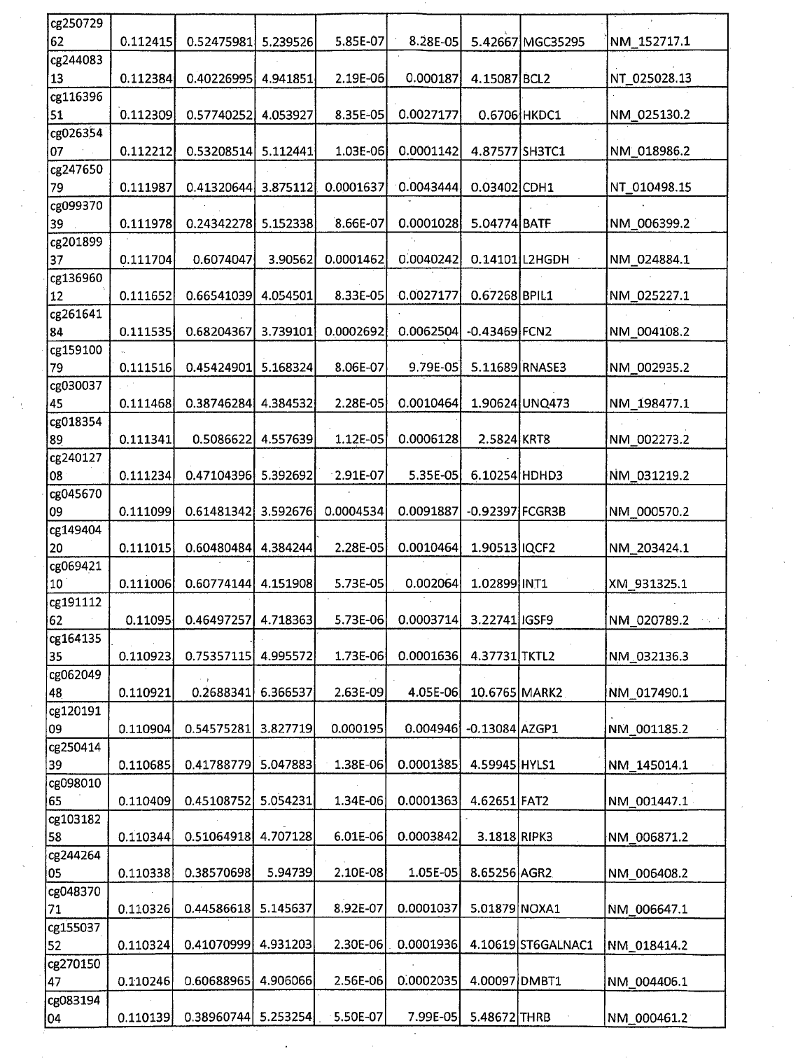

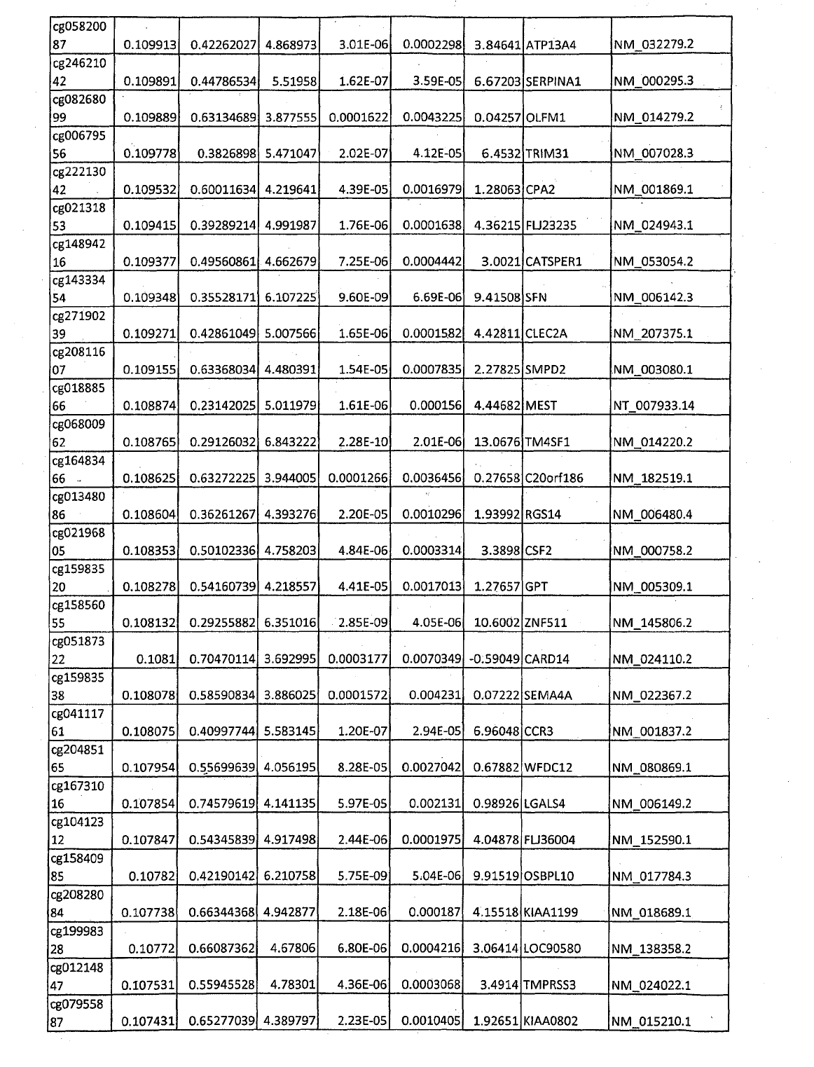

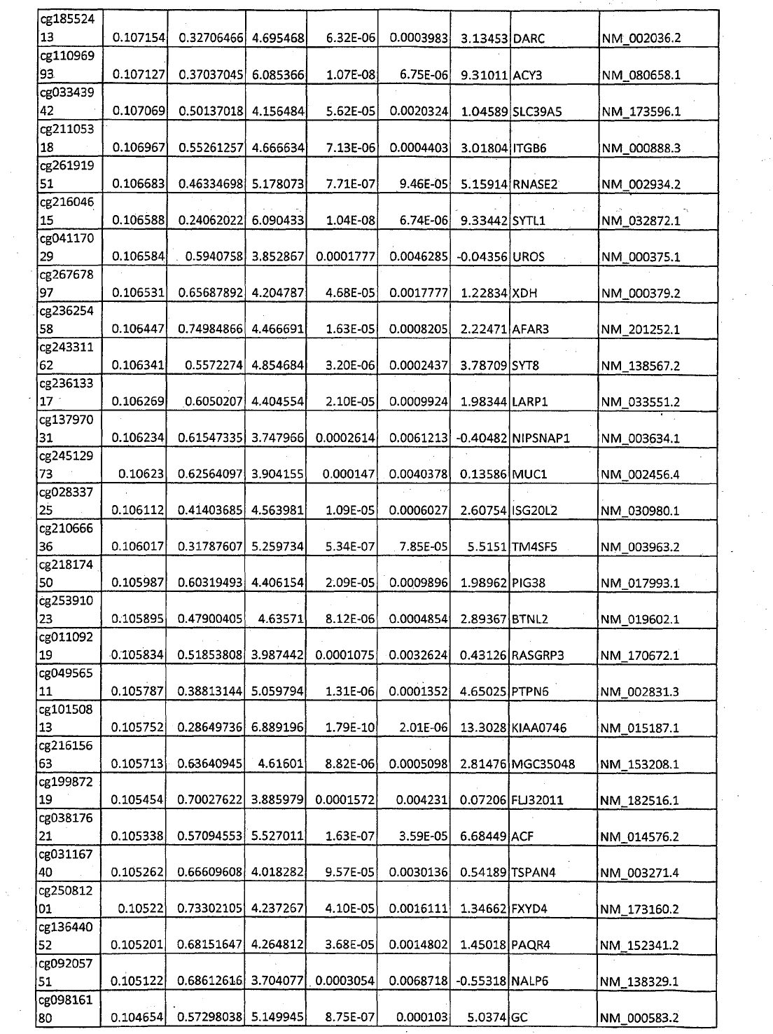

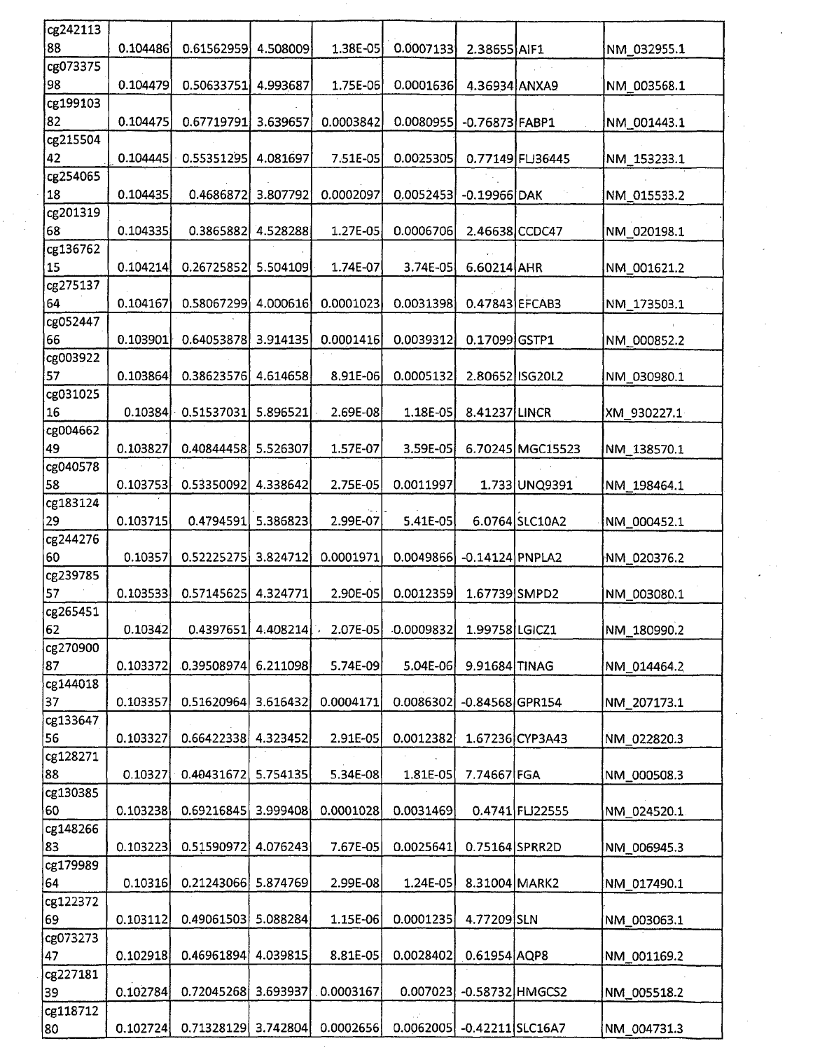

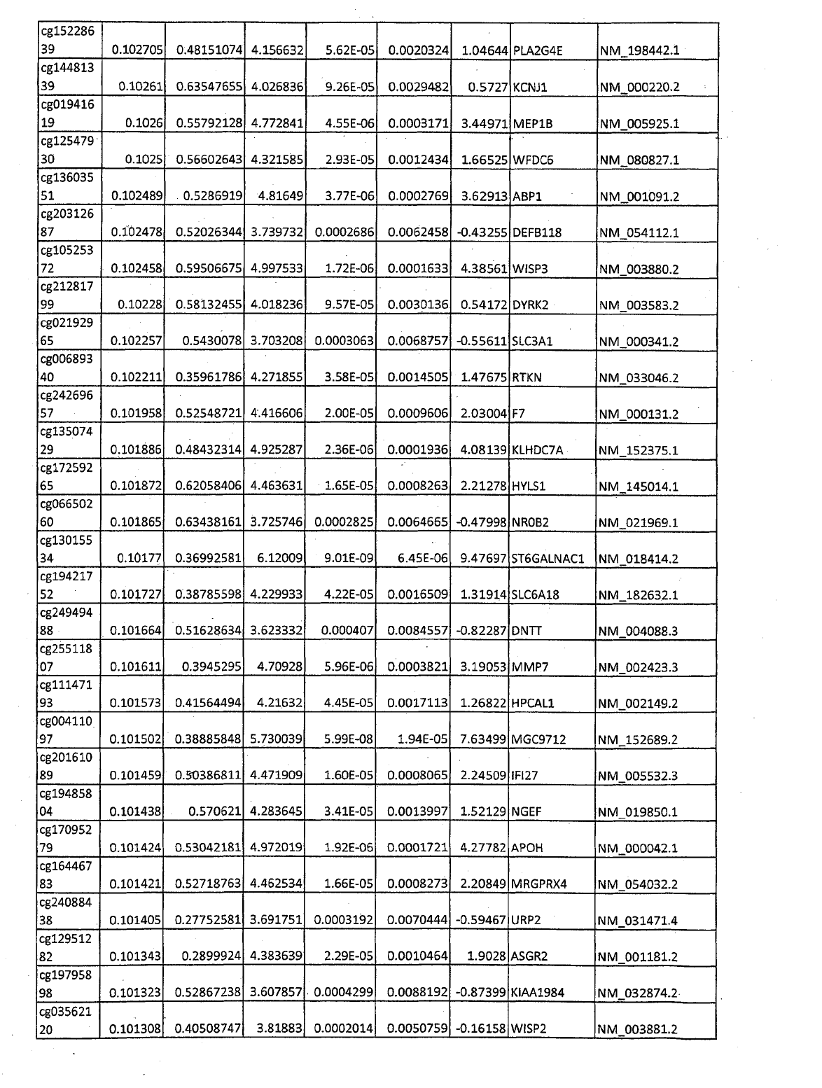

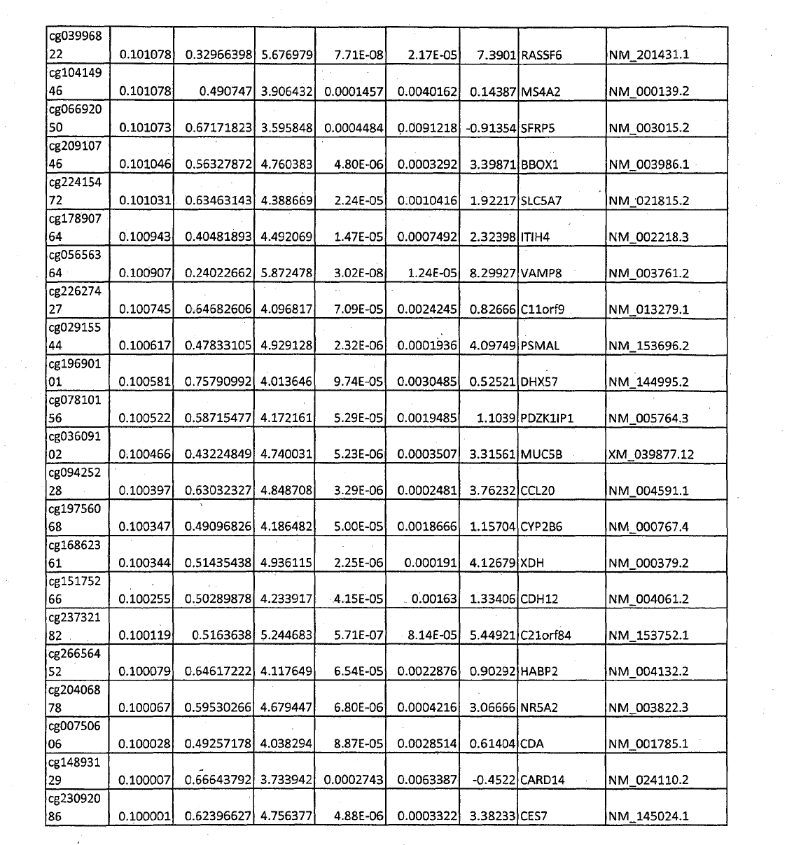

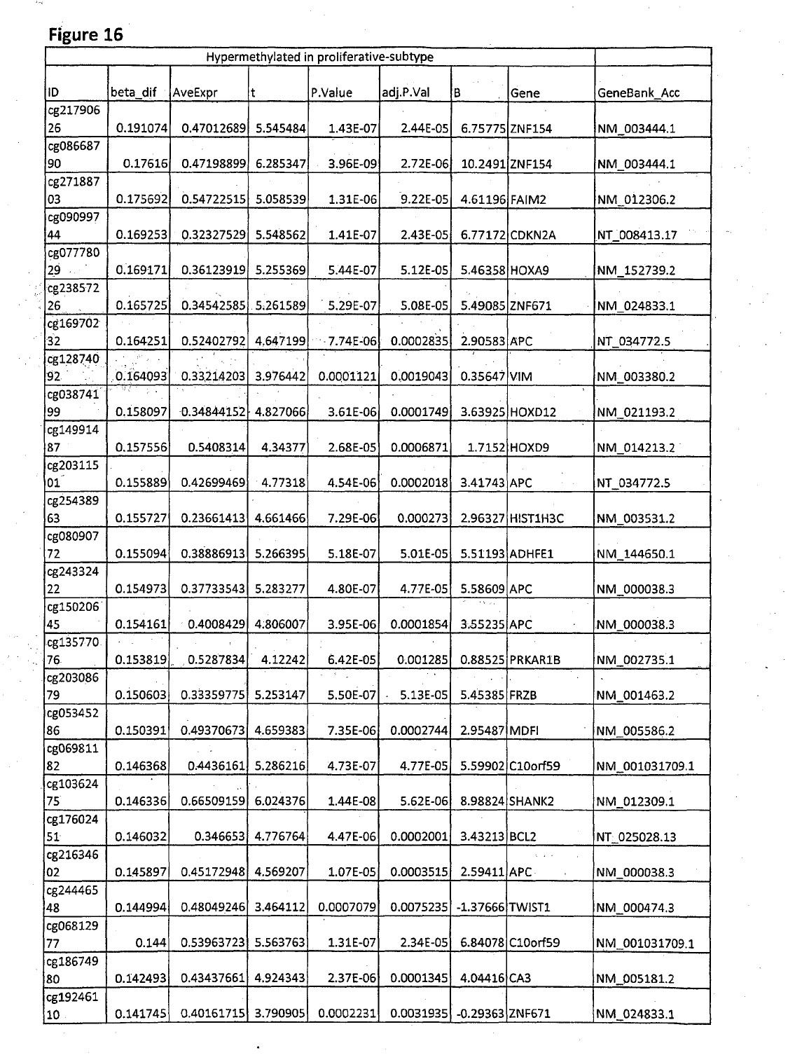

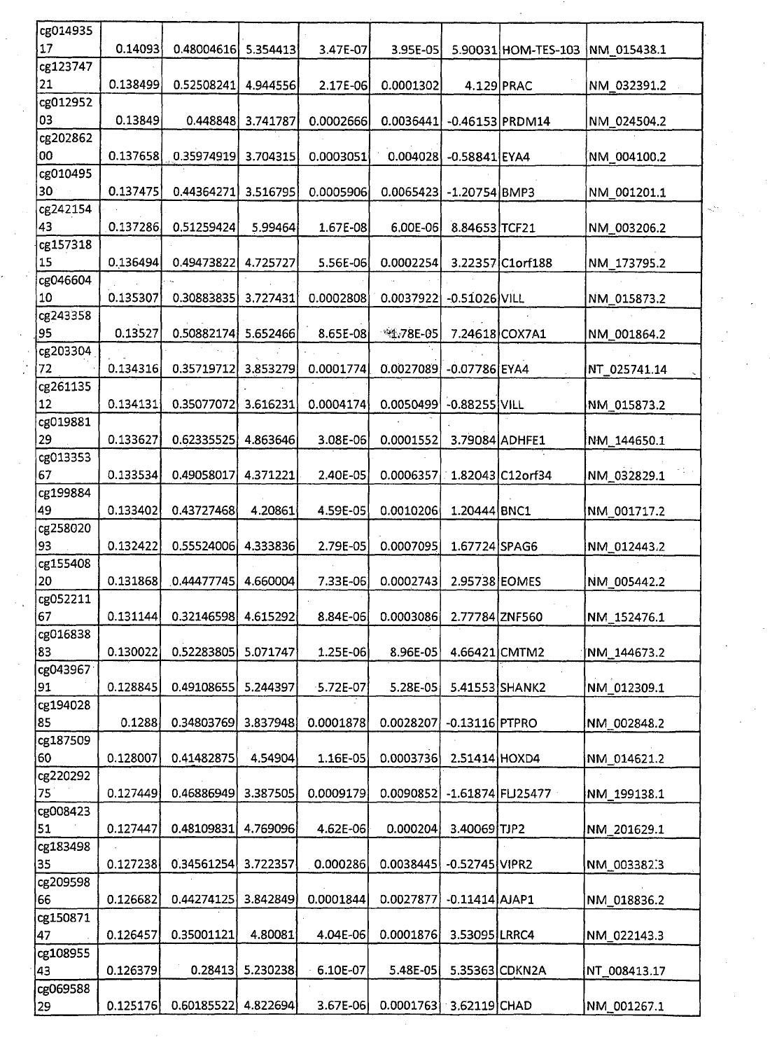

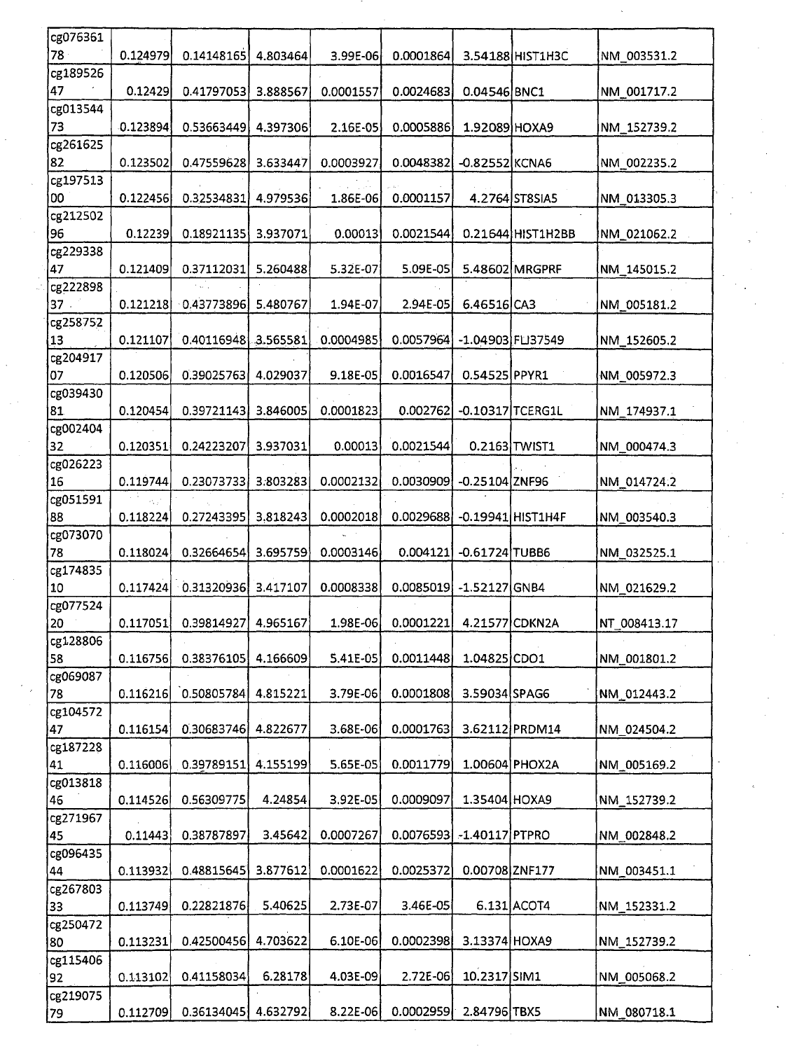

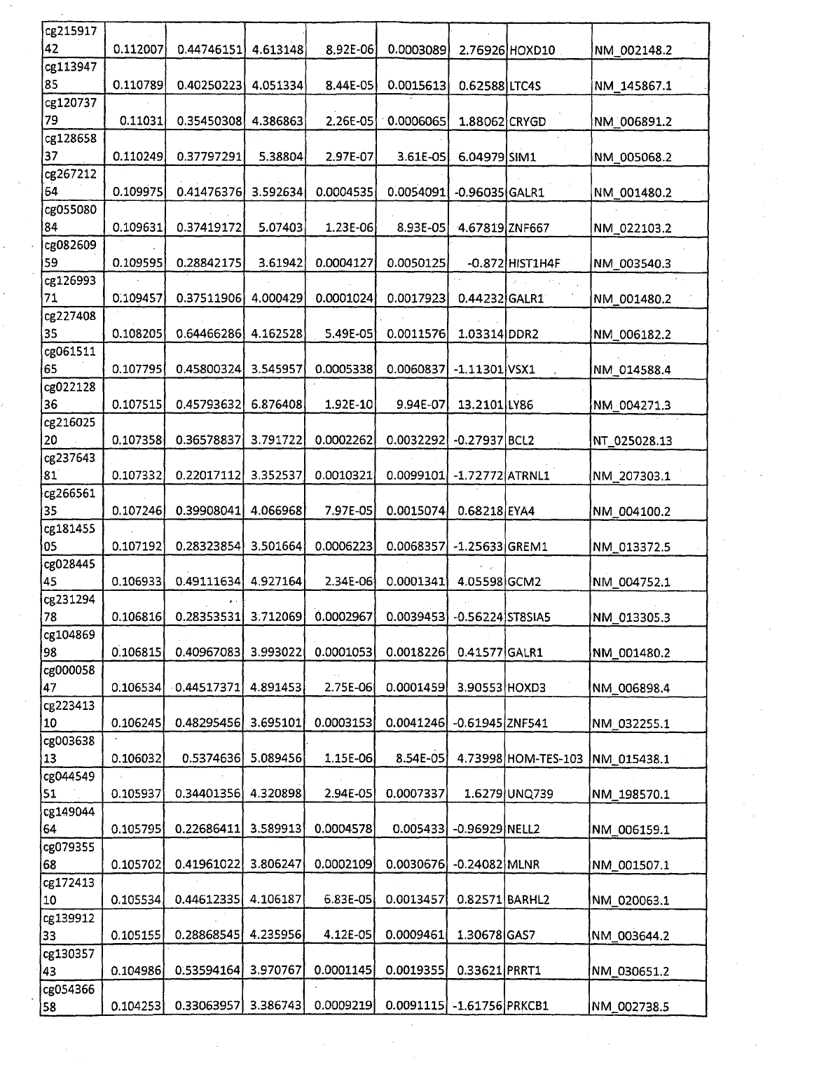

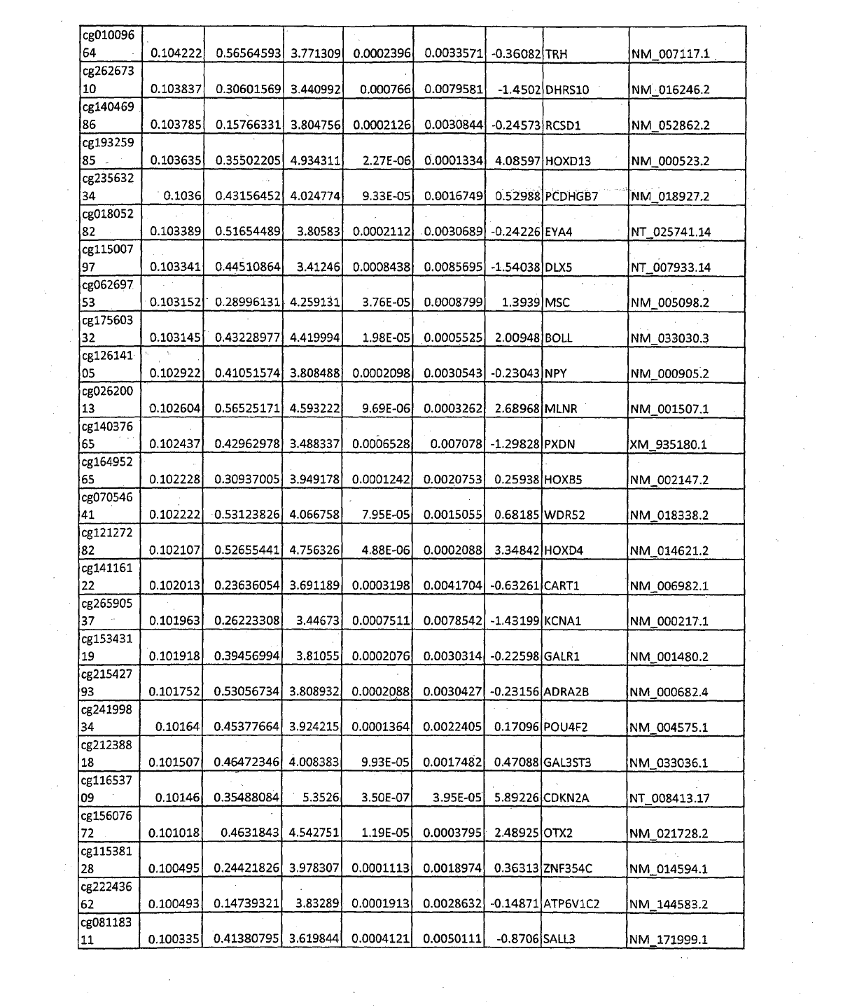

4. The grouping of claim 3, wherein the hypermethylated sites comprise the genes listed in FIG. 14 for the invasive subtype and in FIG. 16 for the proliferative subtype.

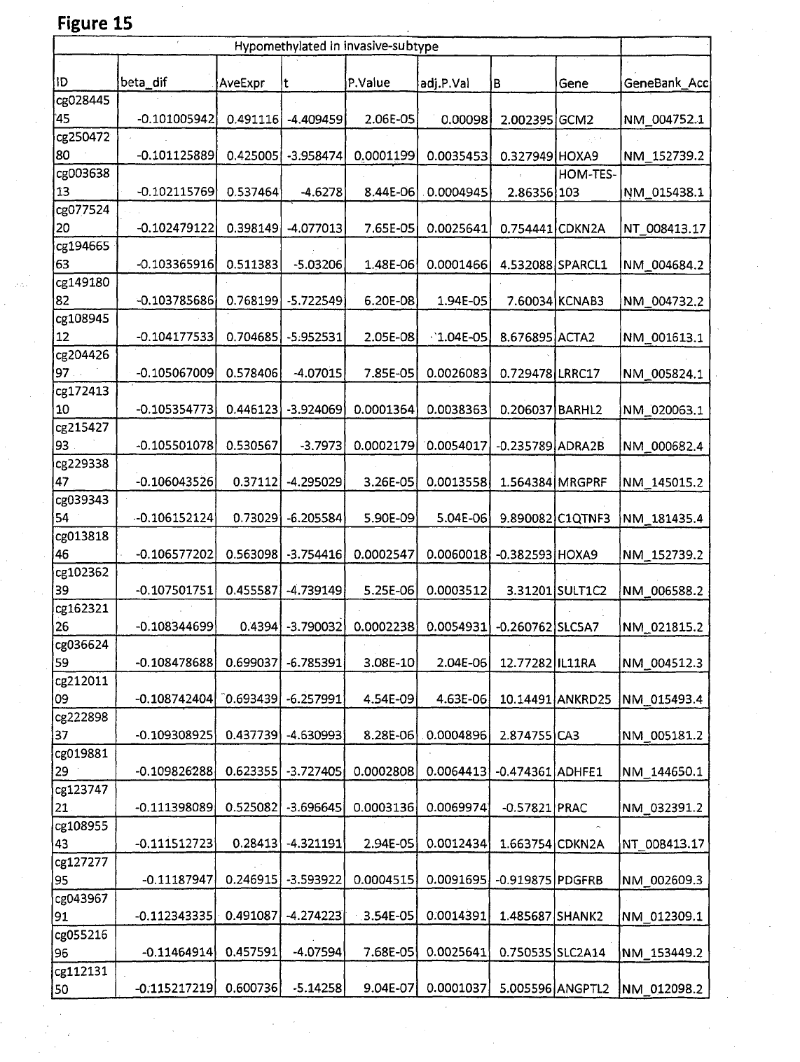

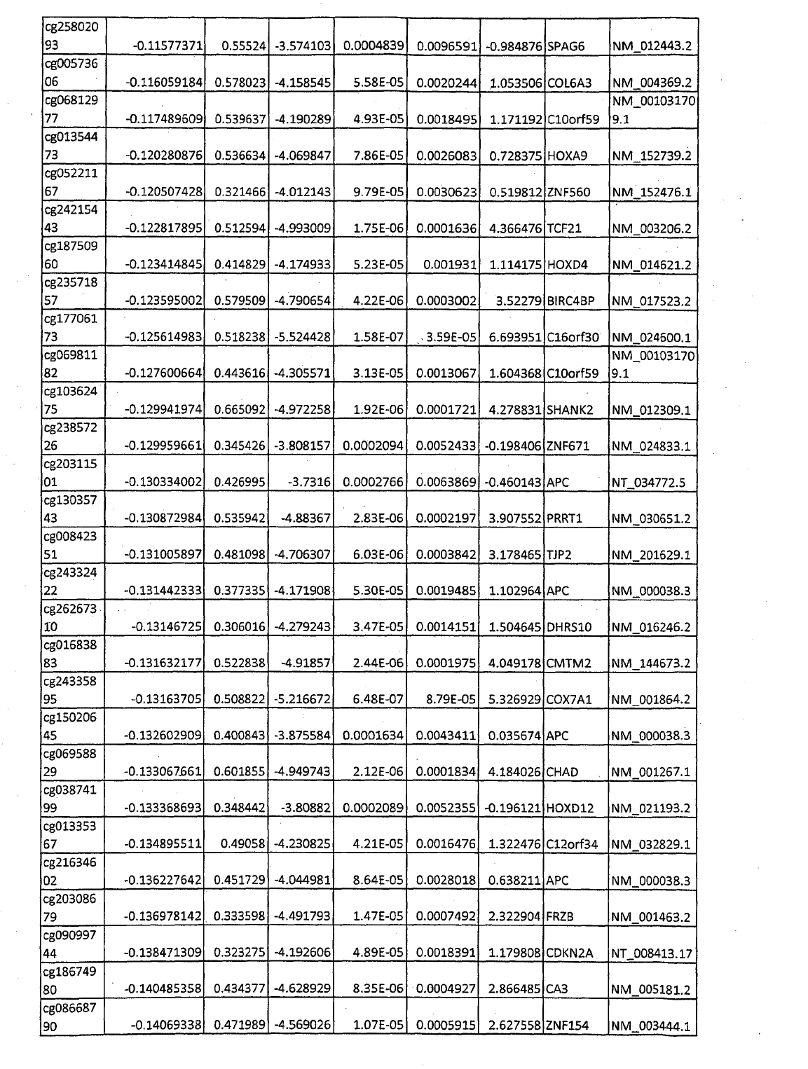

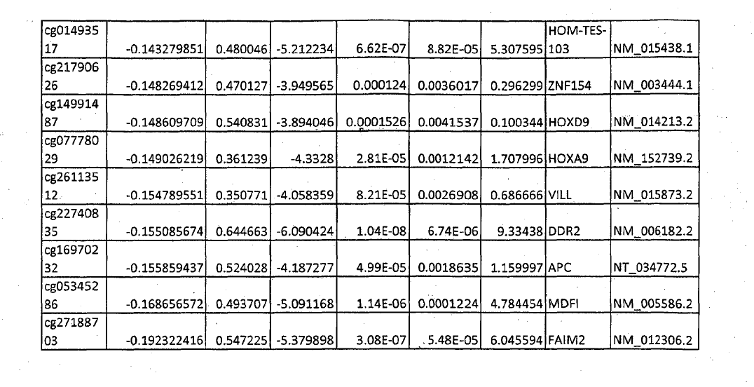

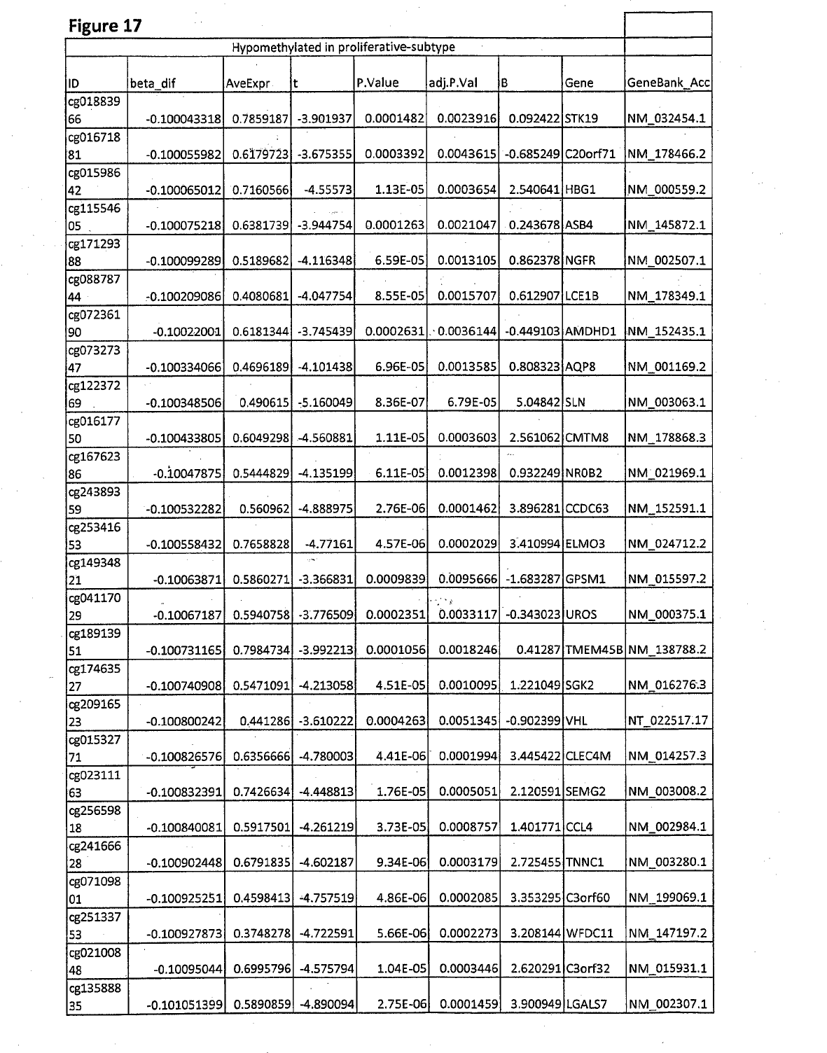

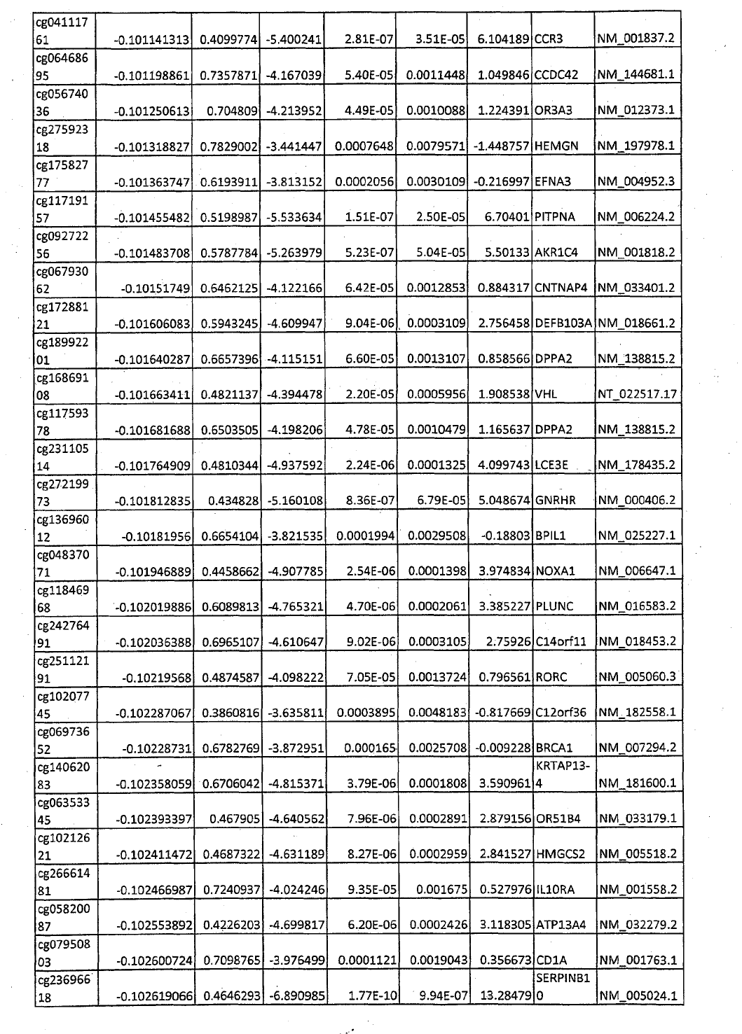

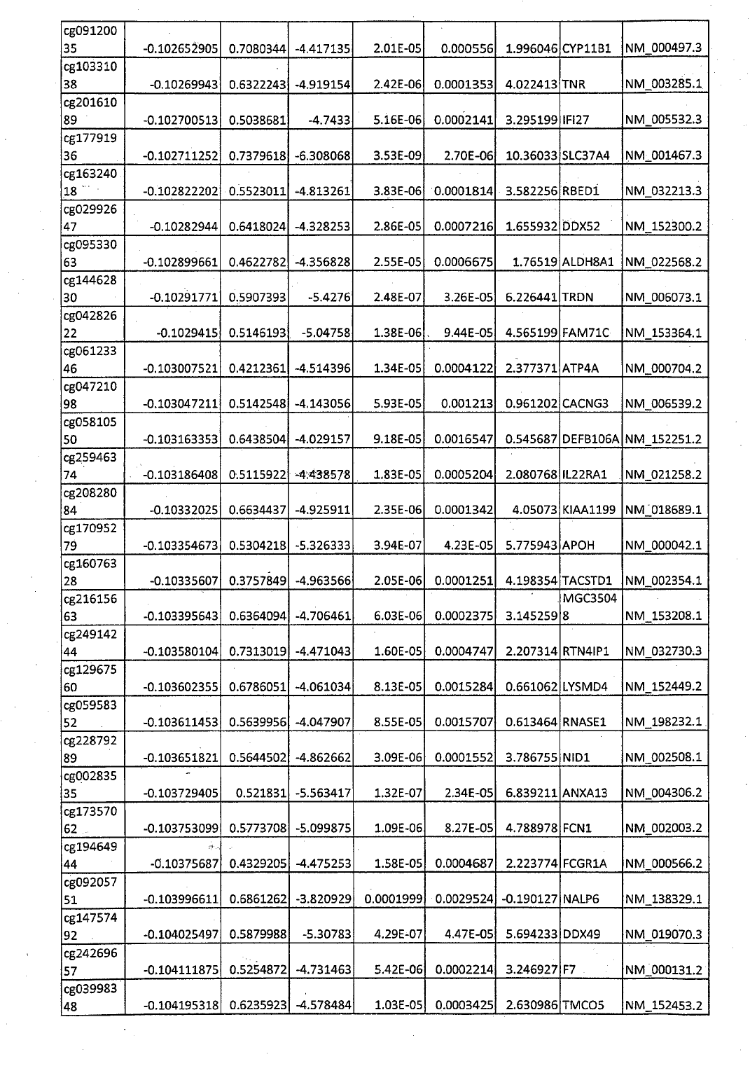

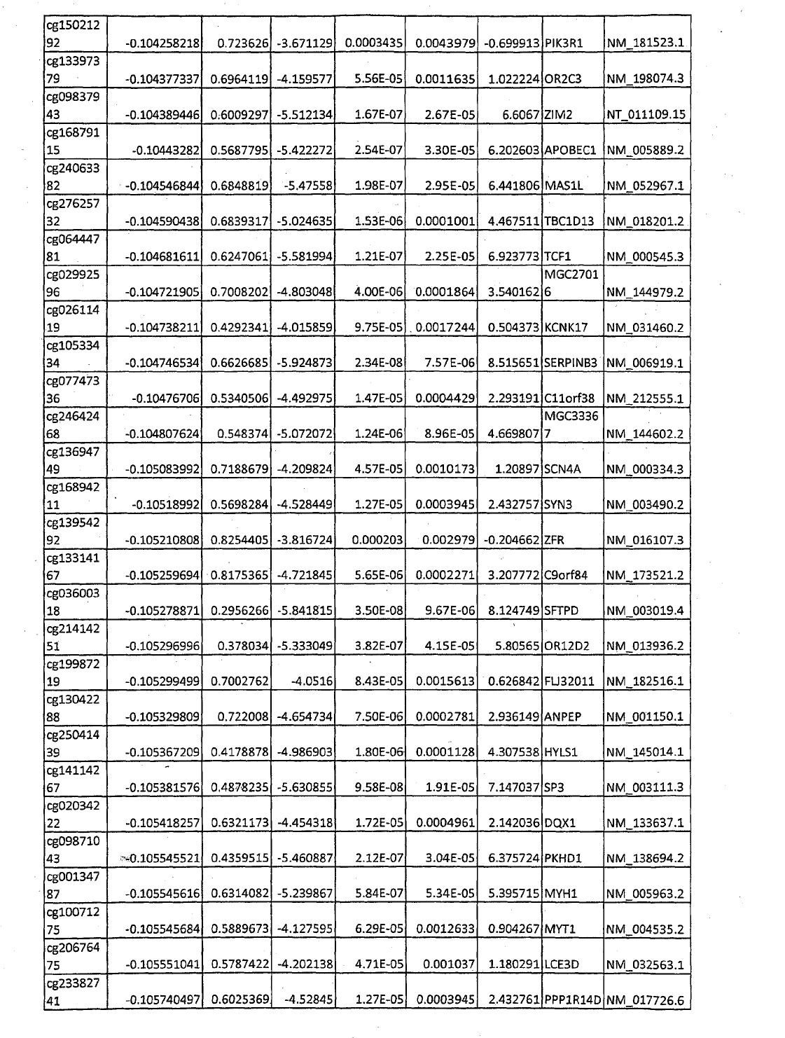

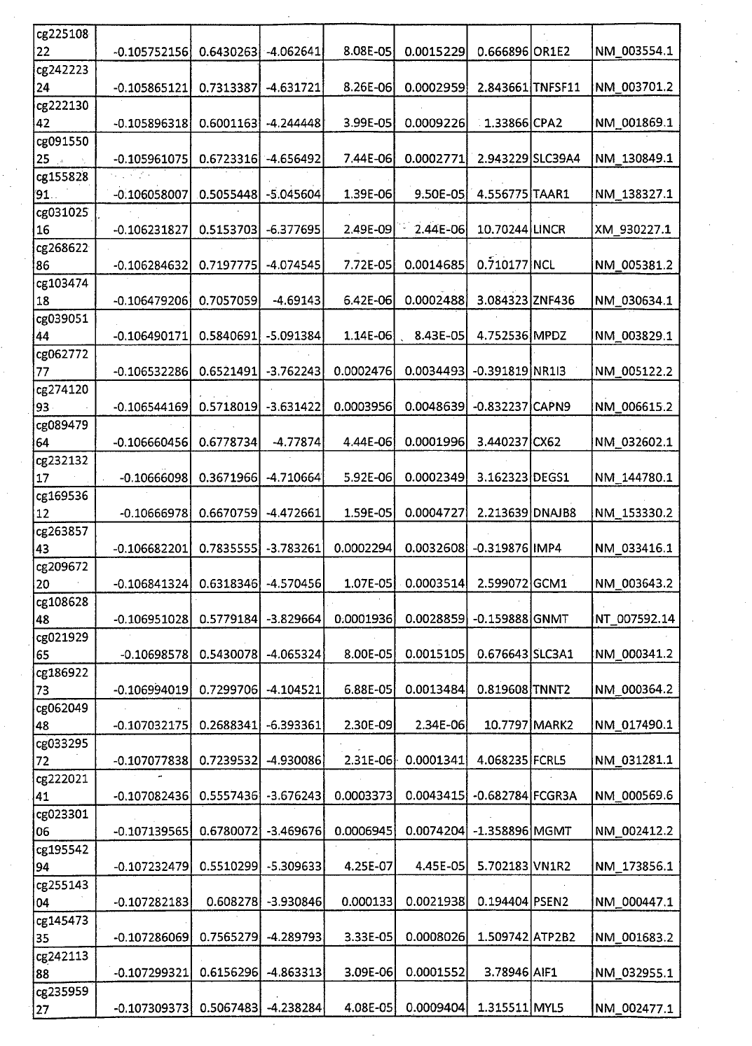

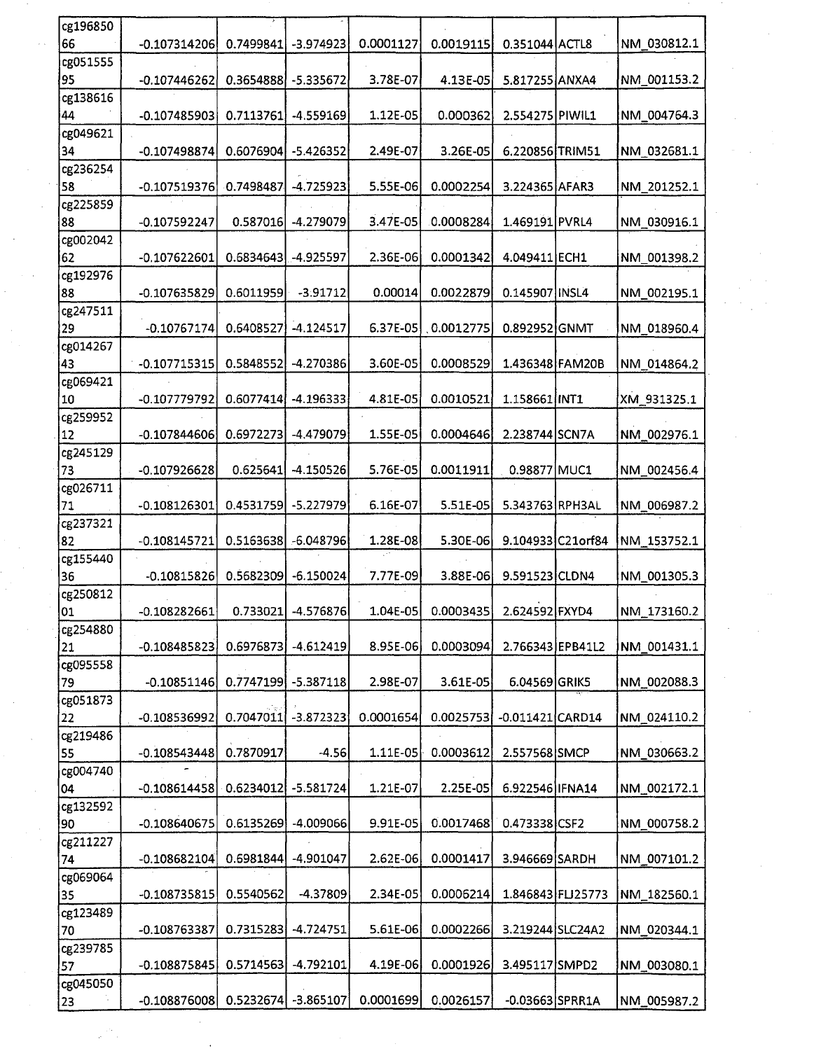

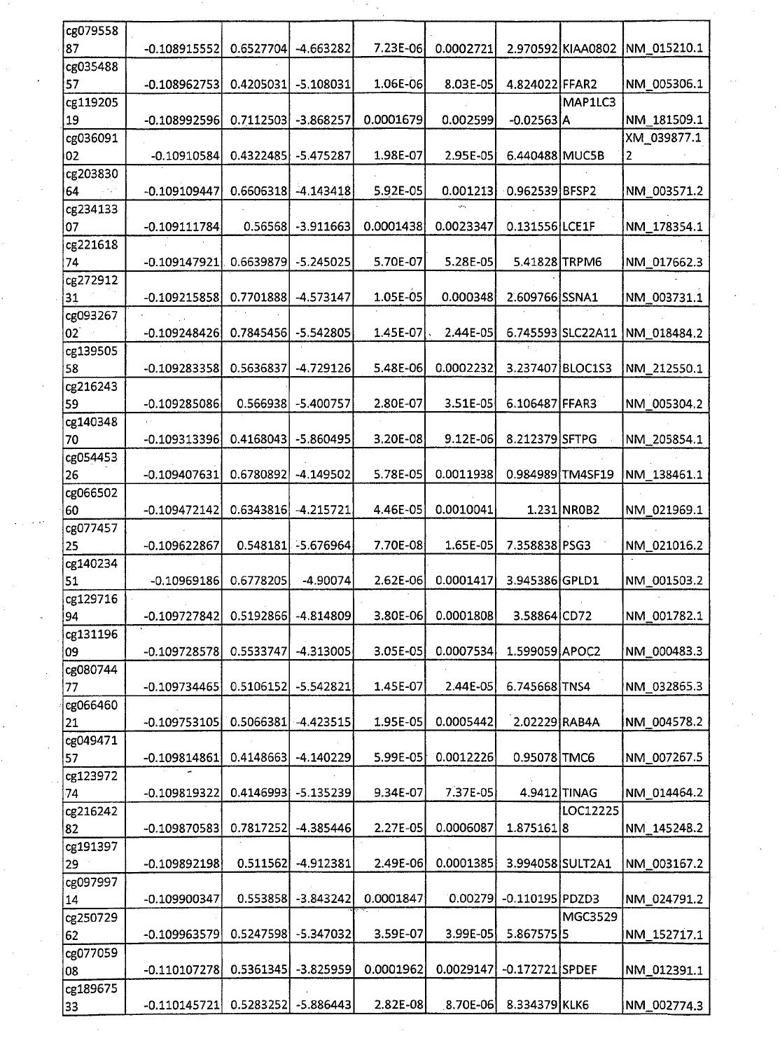

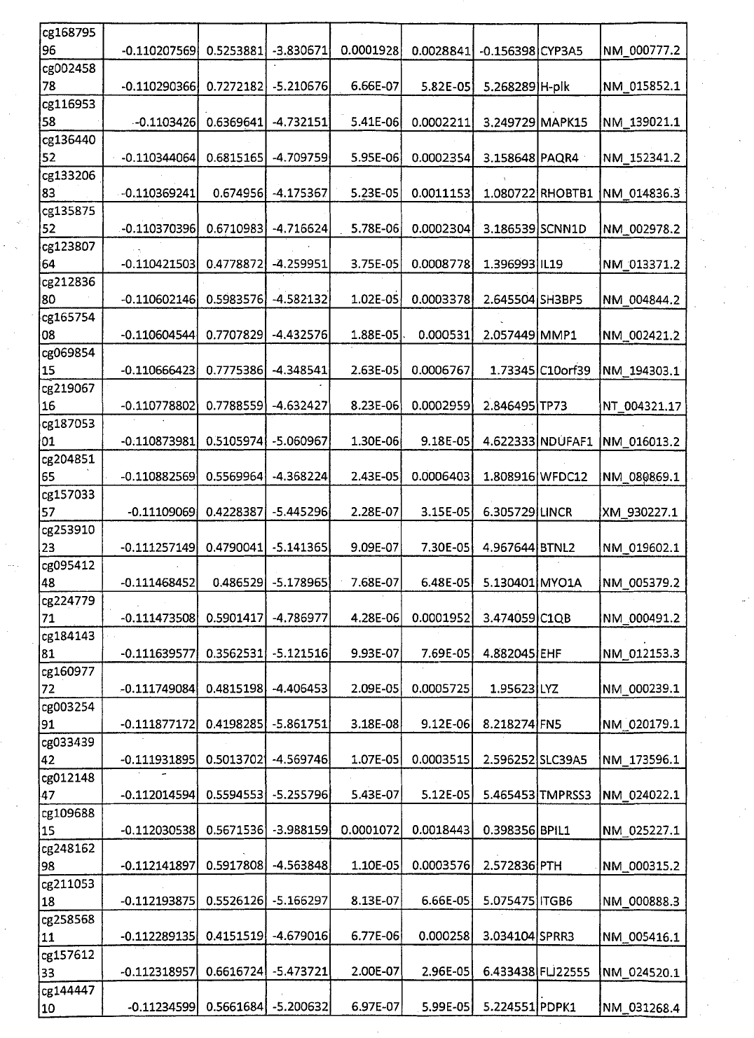

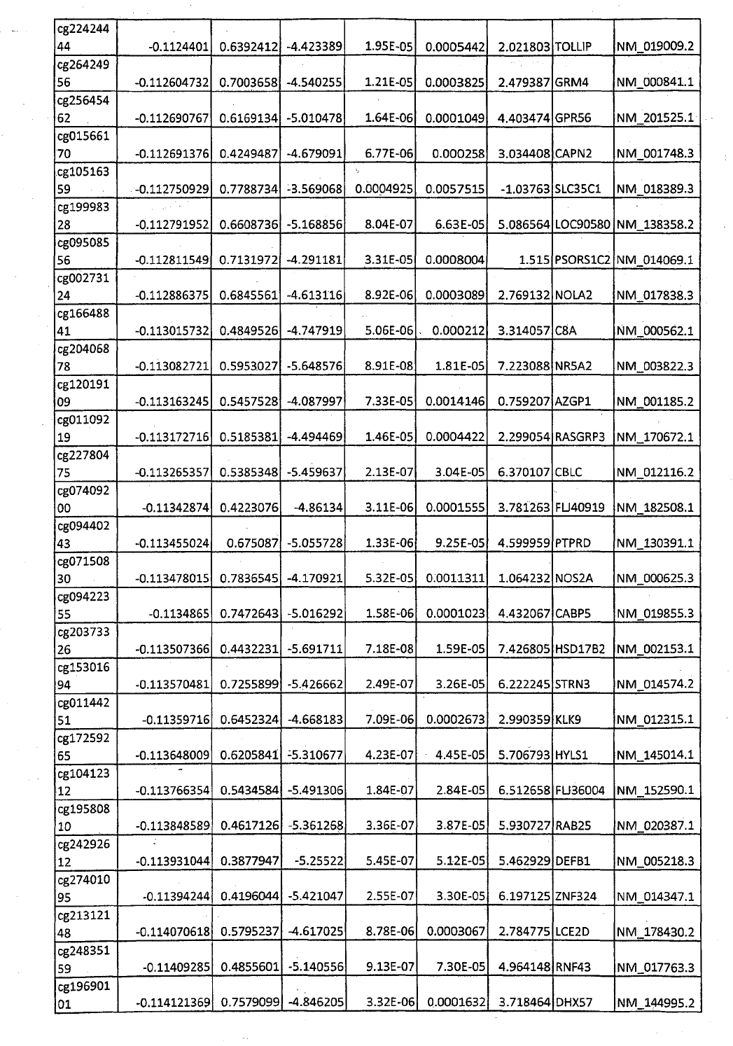

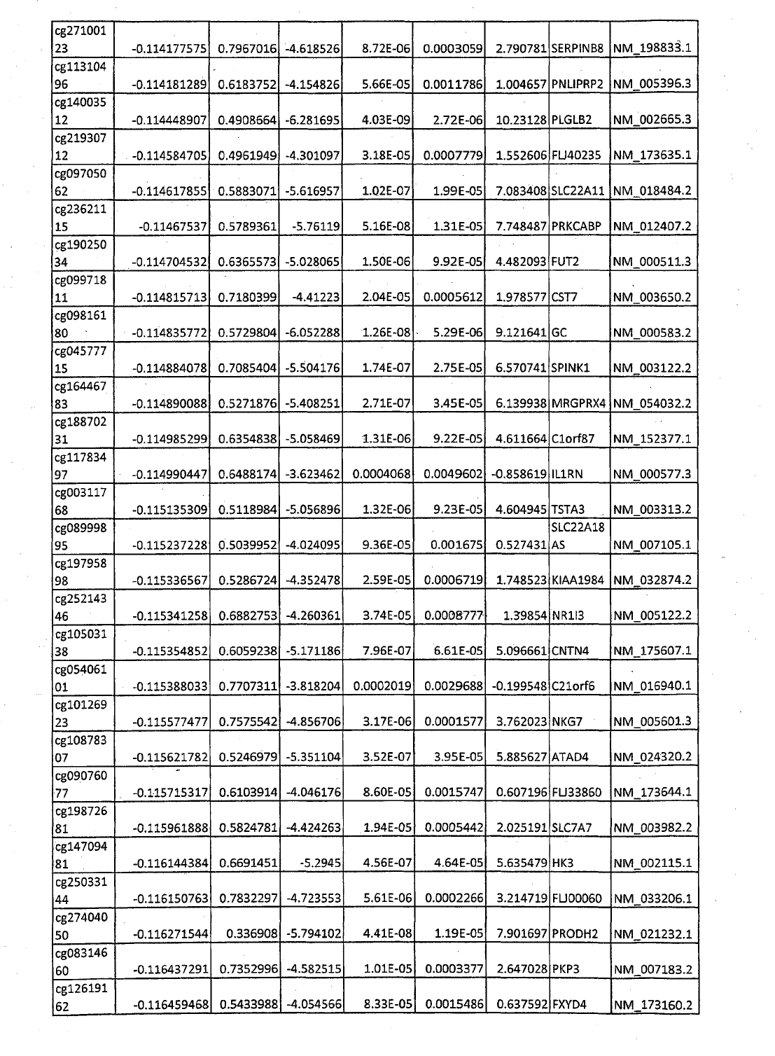

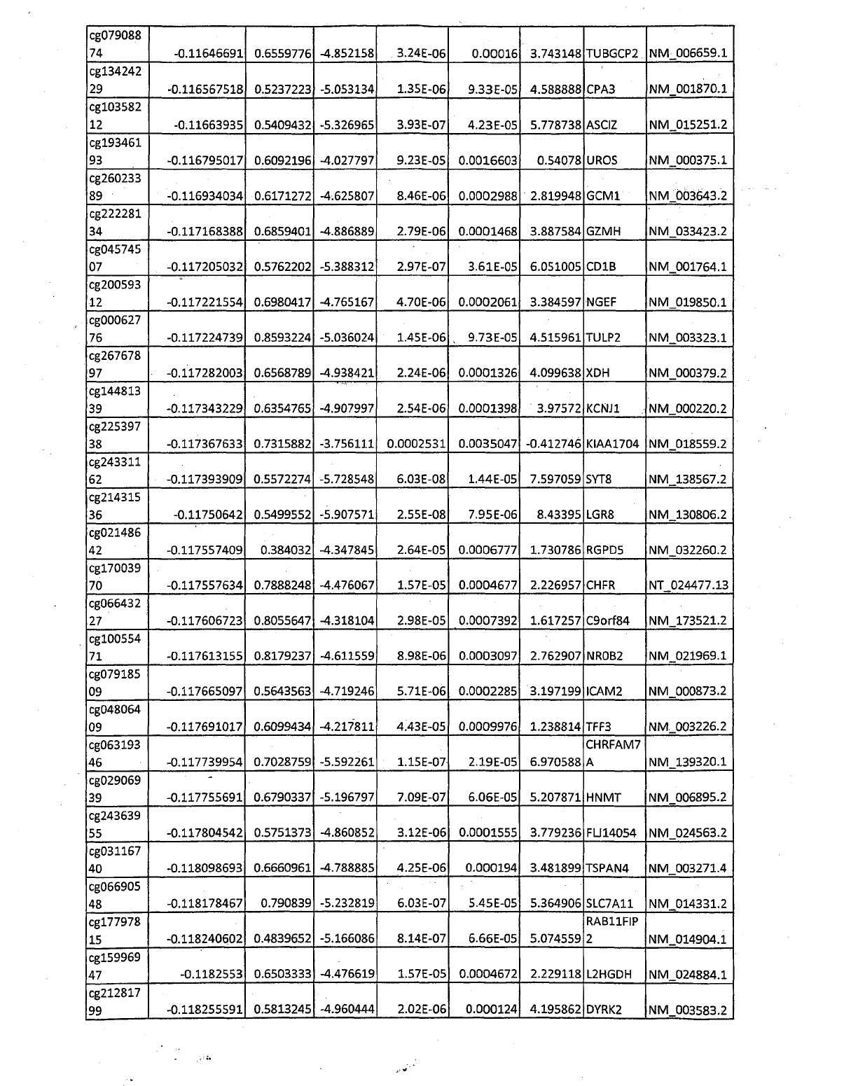

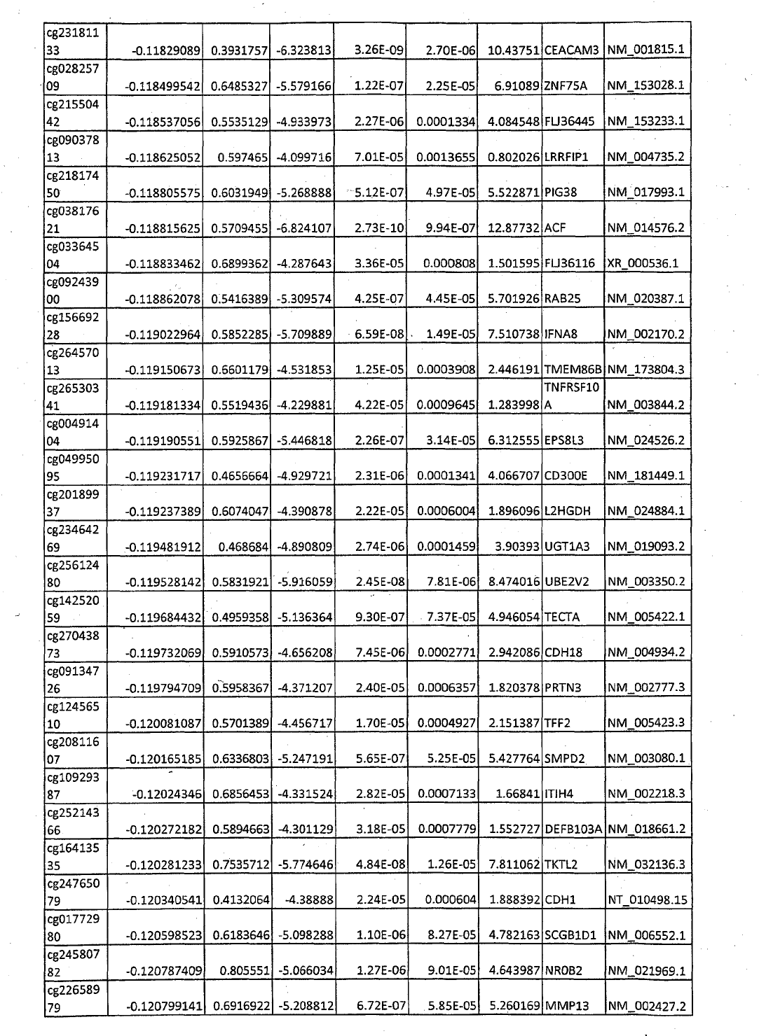

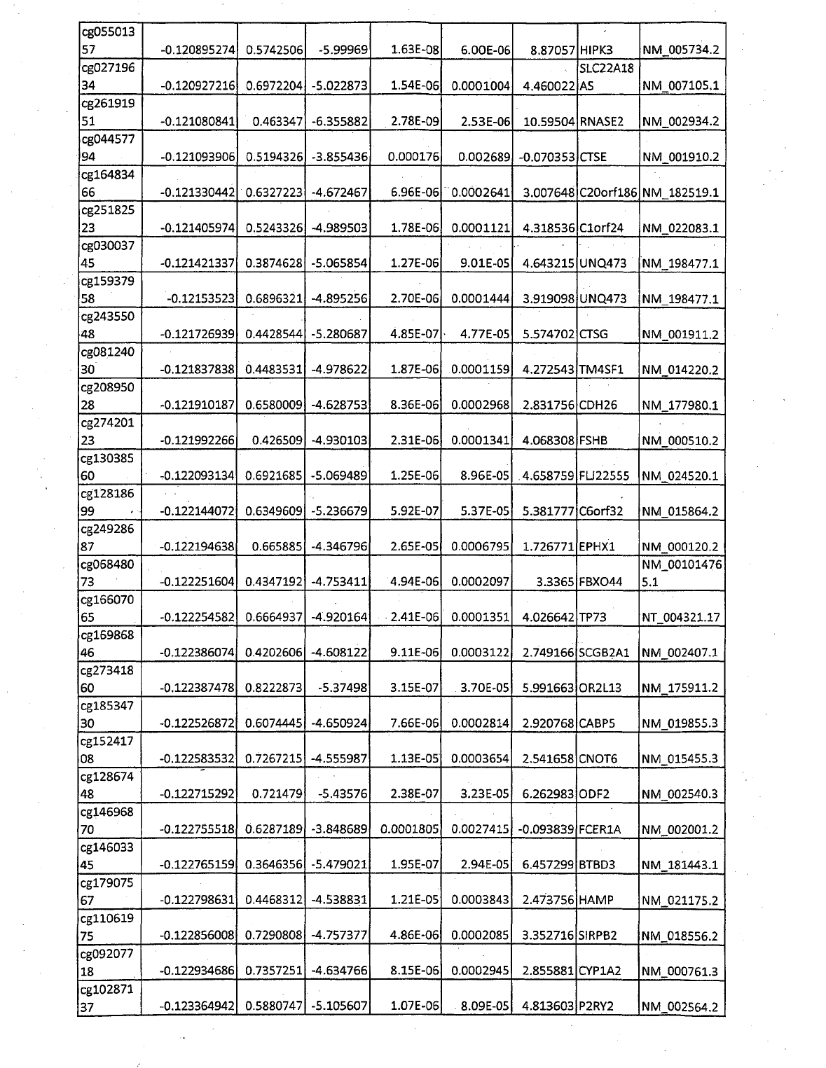

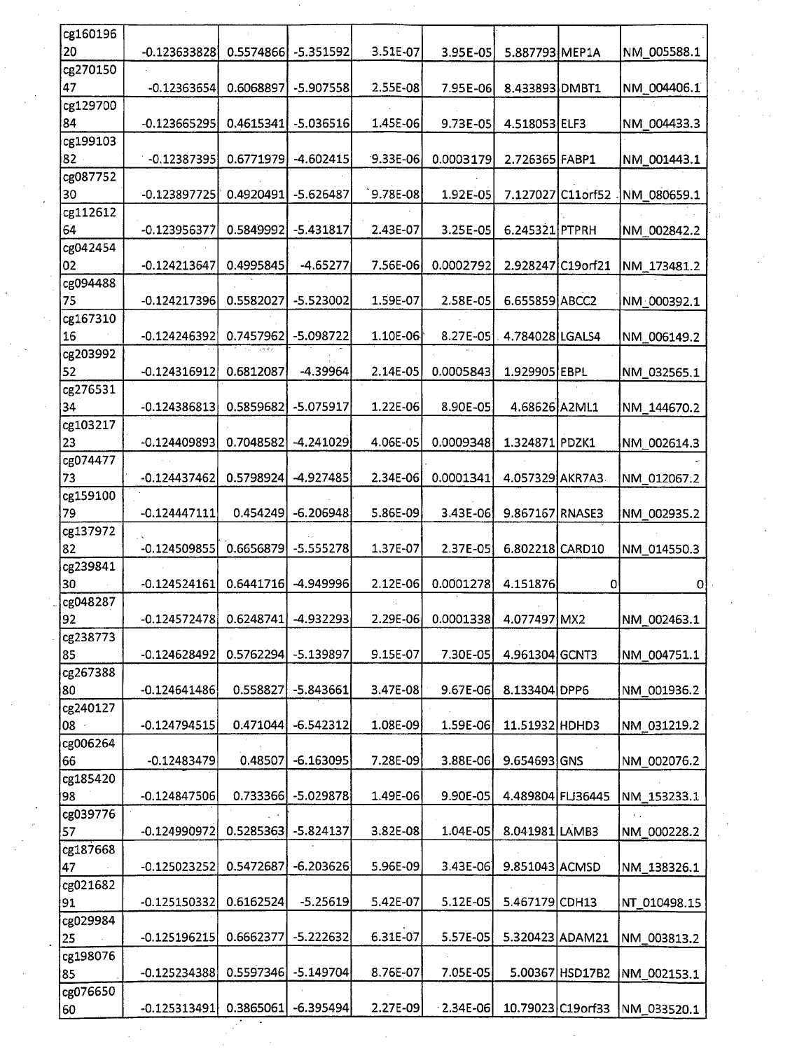

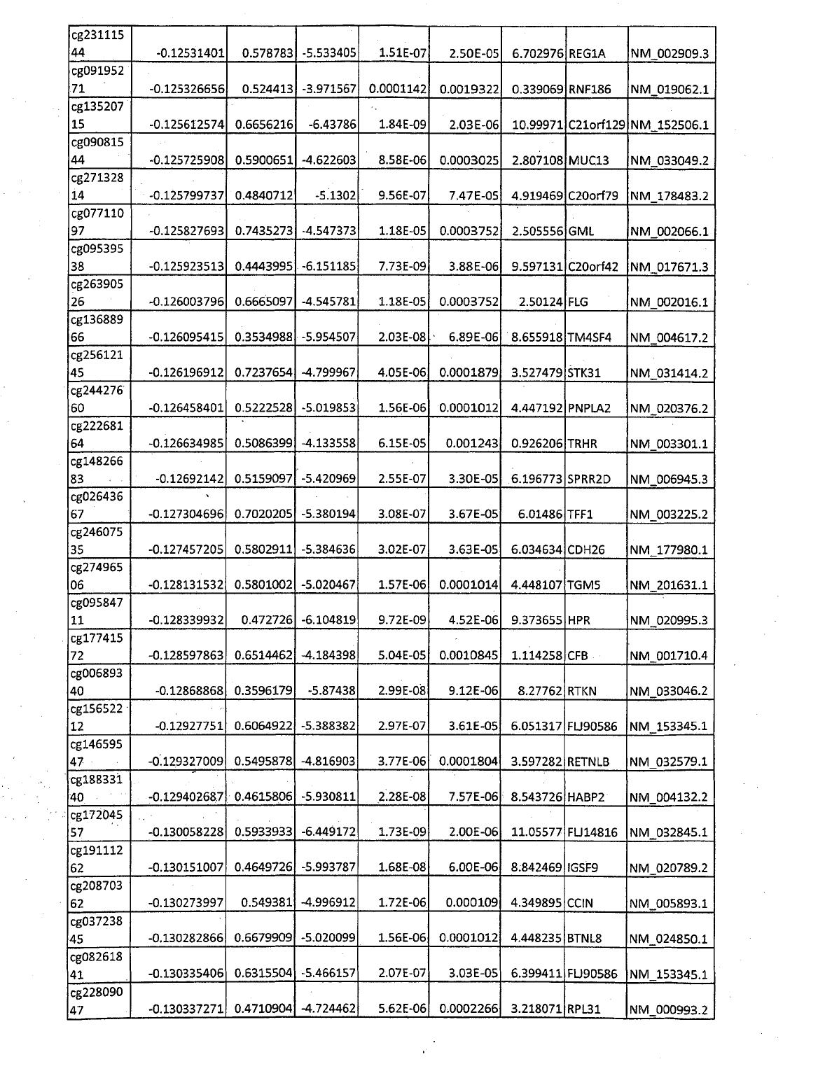

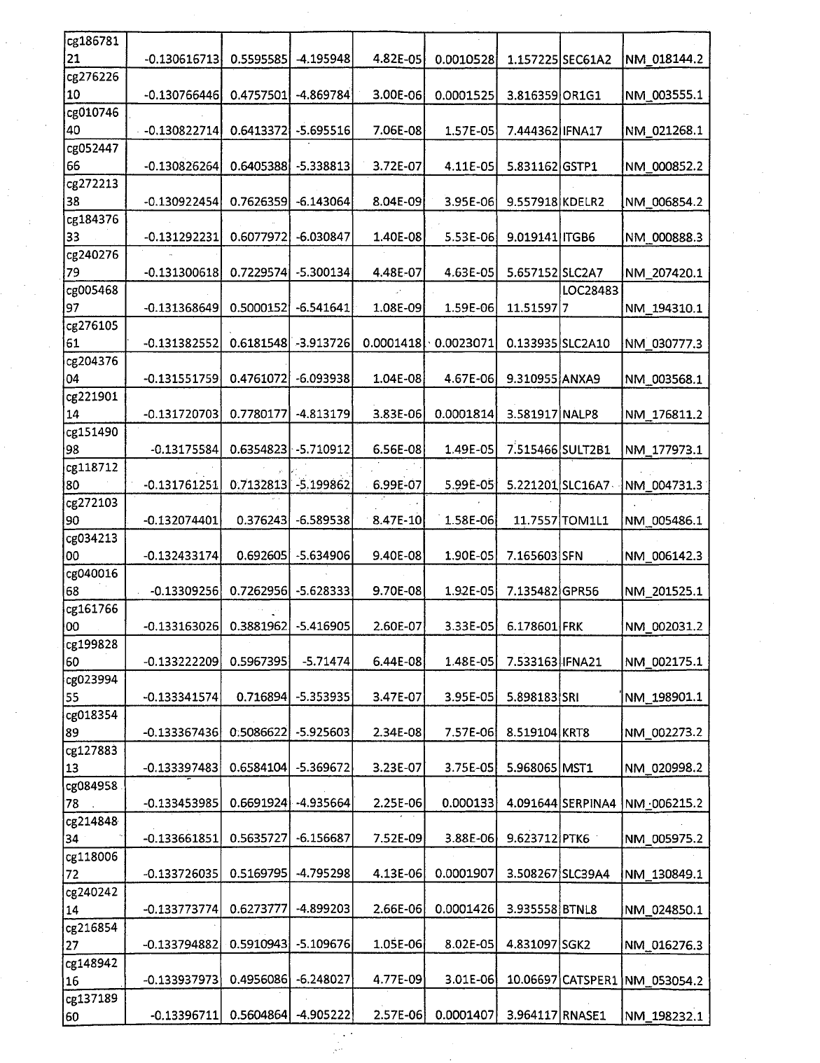

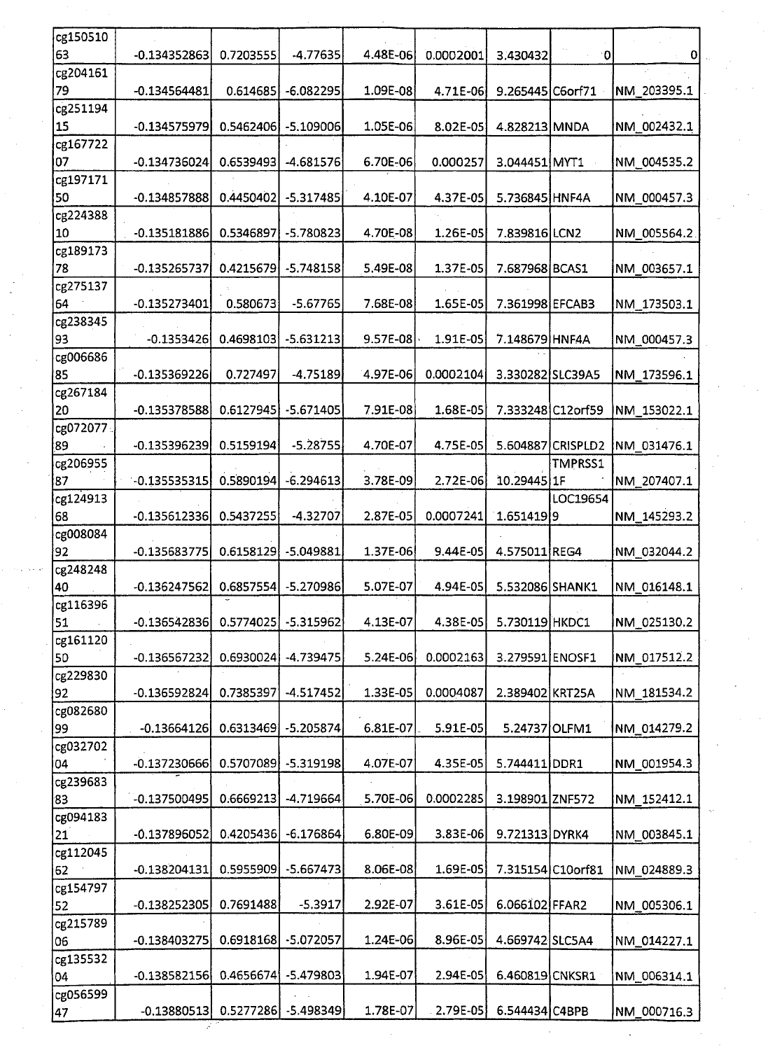

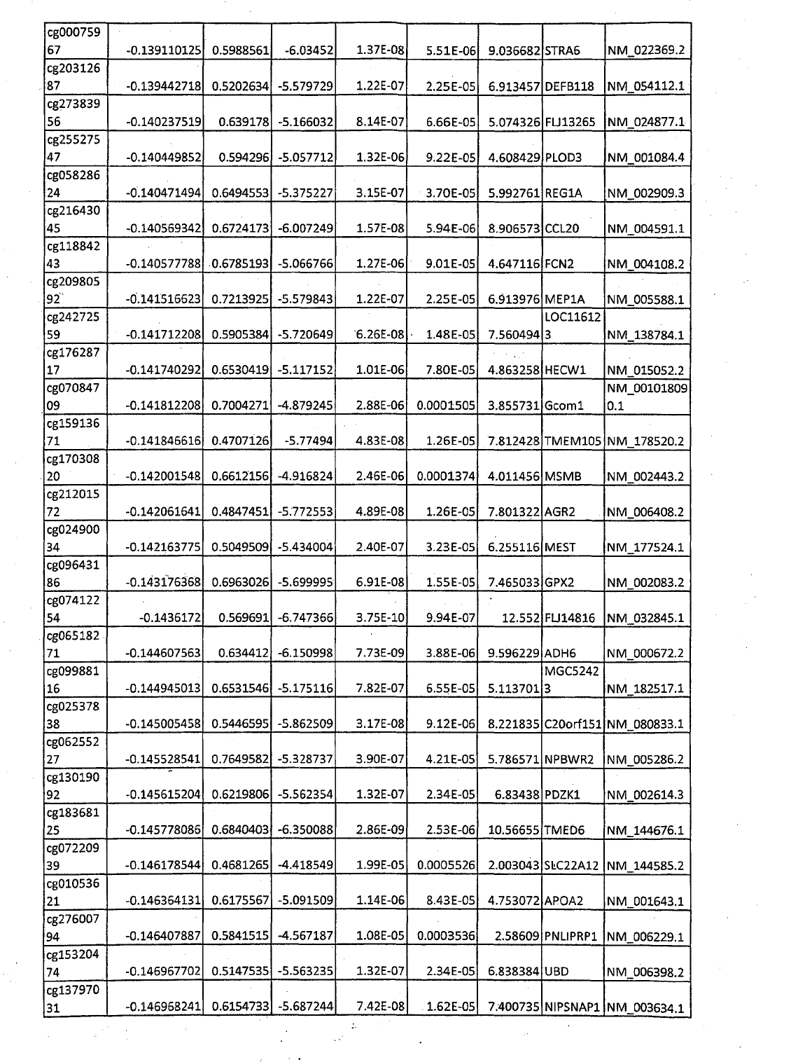

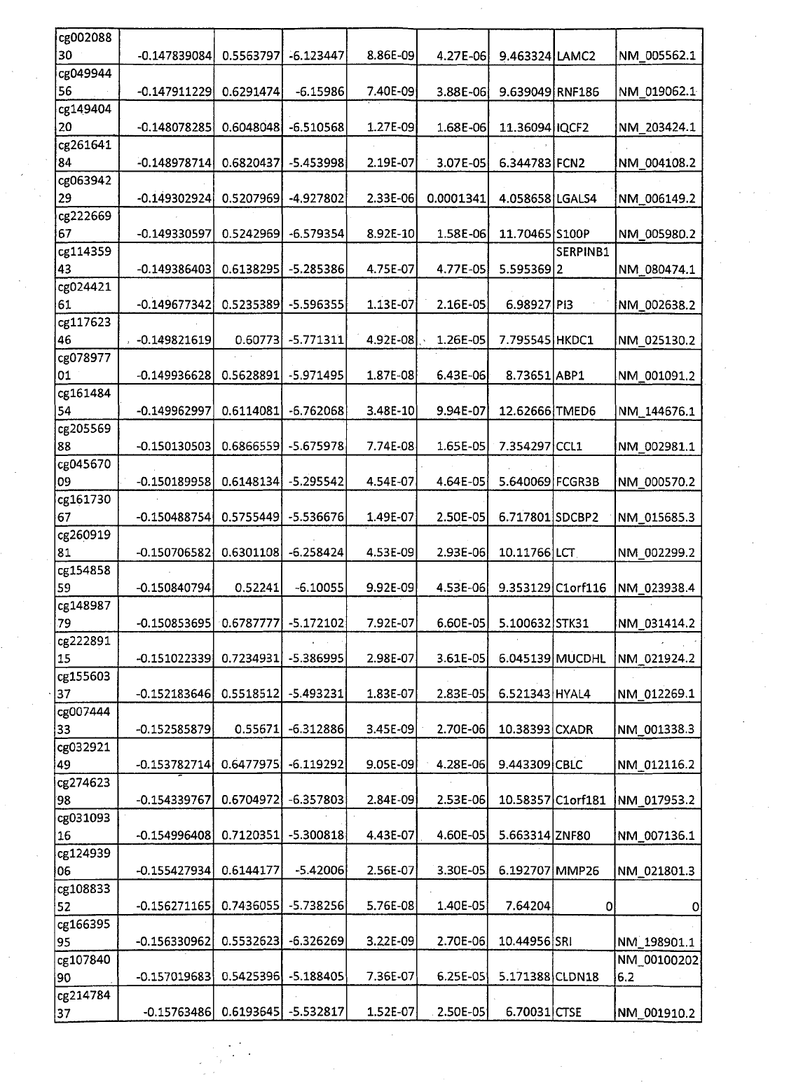

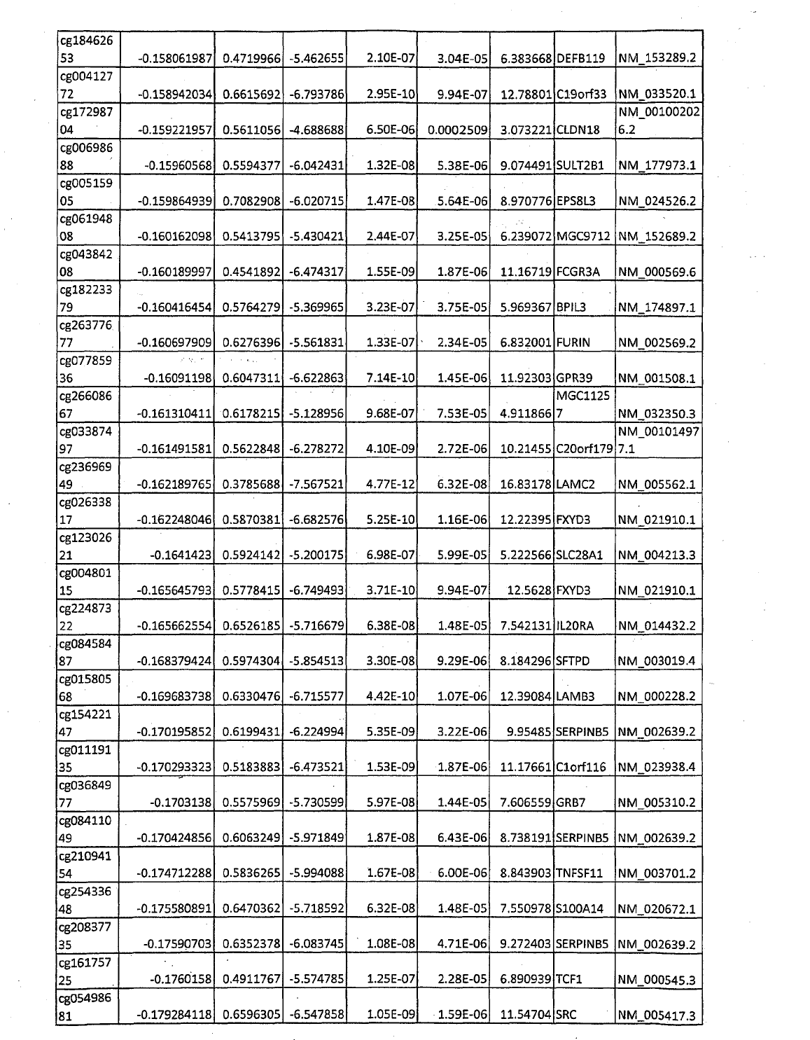

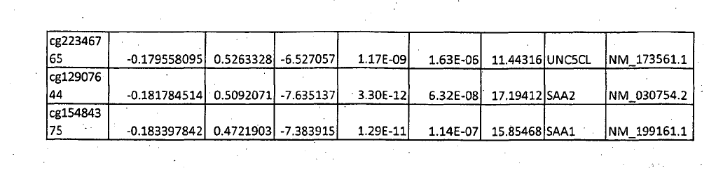

5. The grouping of claim 3, wherein the hypomethylated sites comprise the genes listed in FIG. 15 for the invasive subtype and in FIG. 17 for the proliferative subtype.

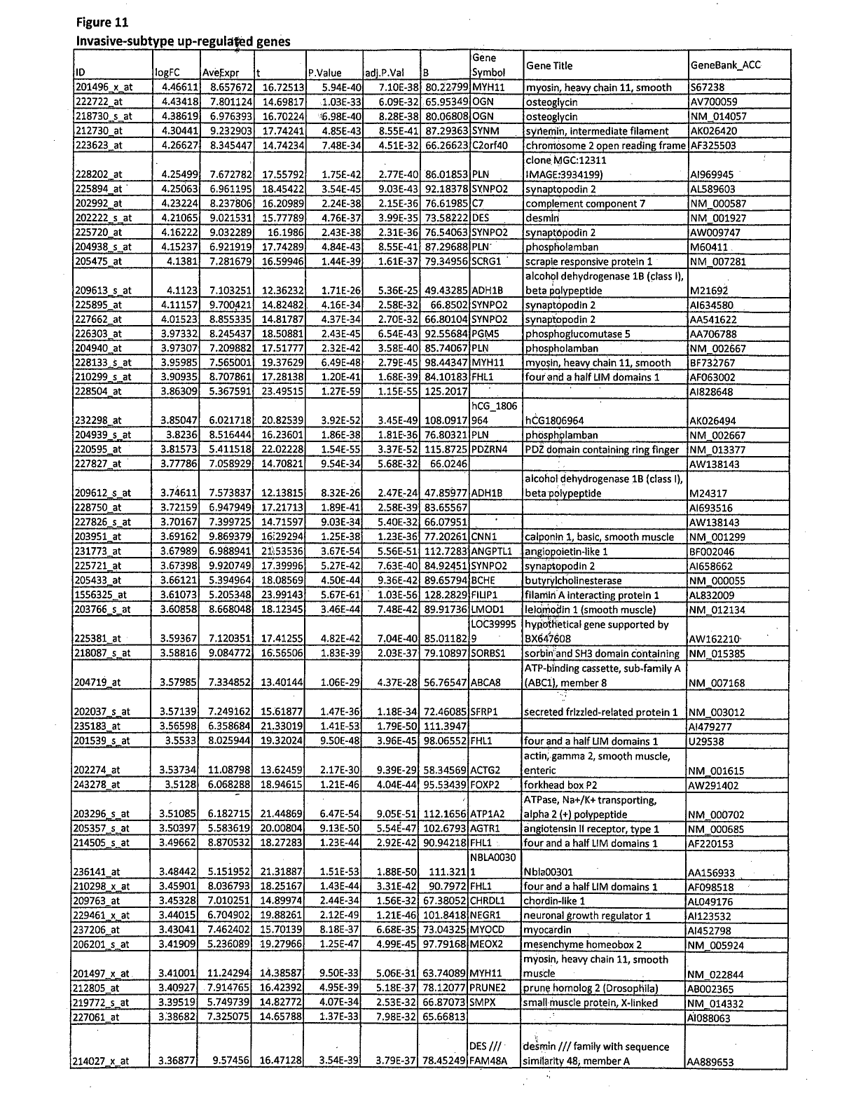

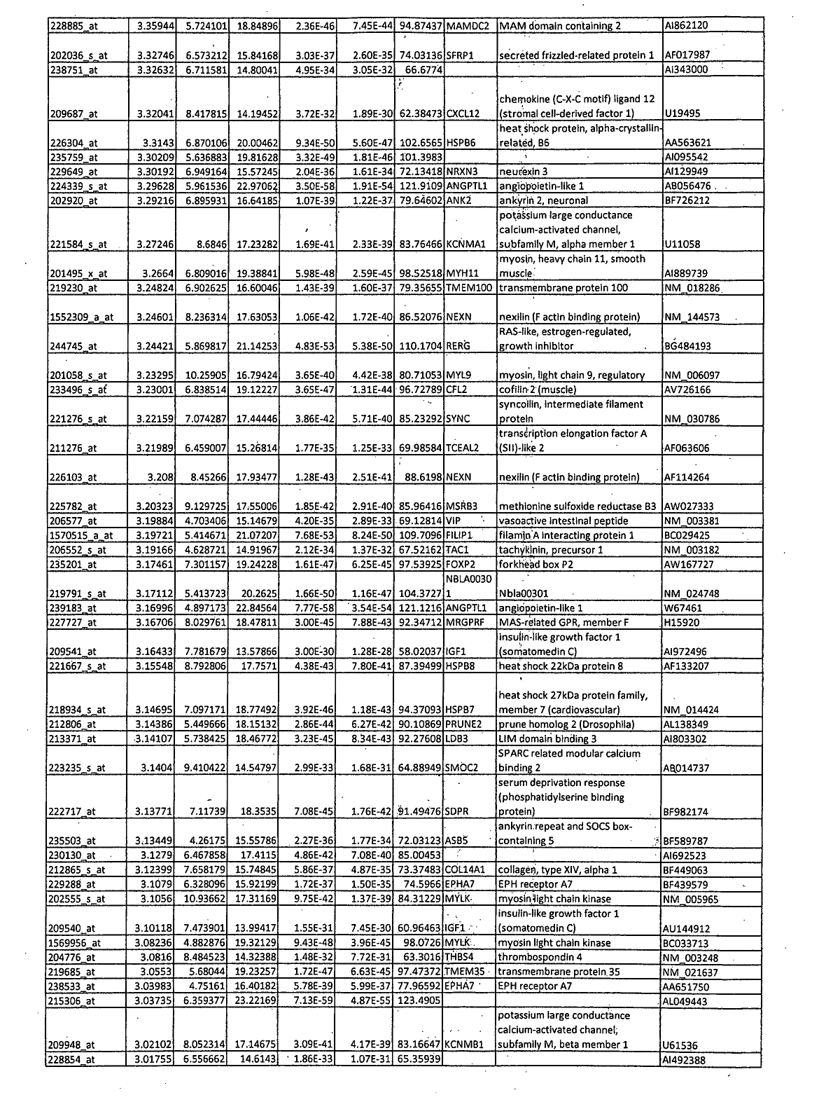

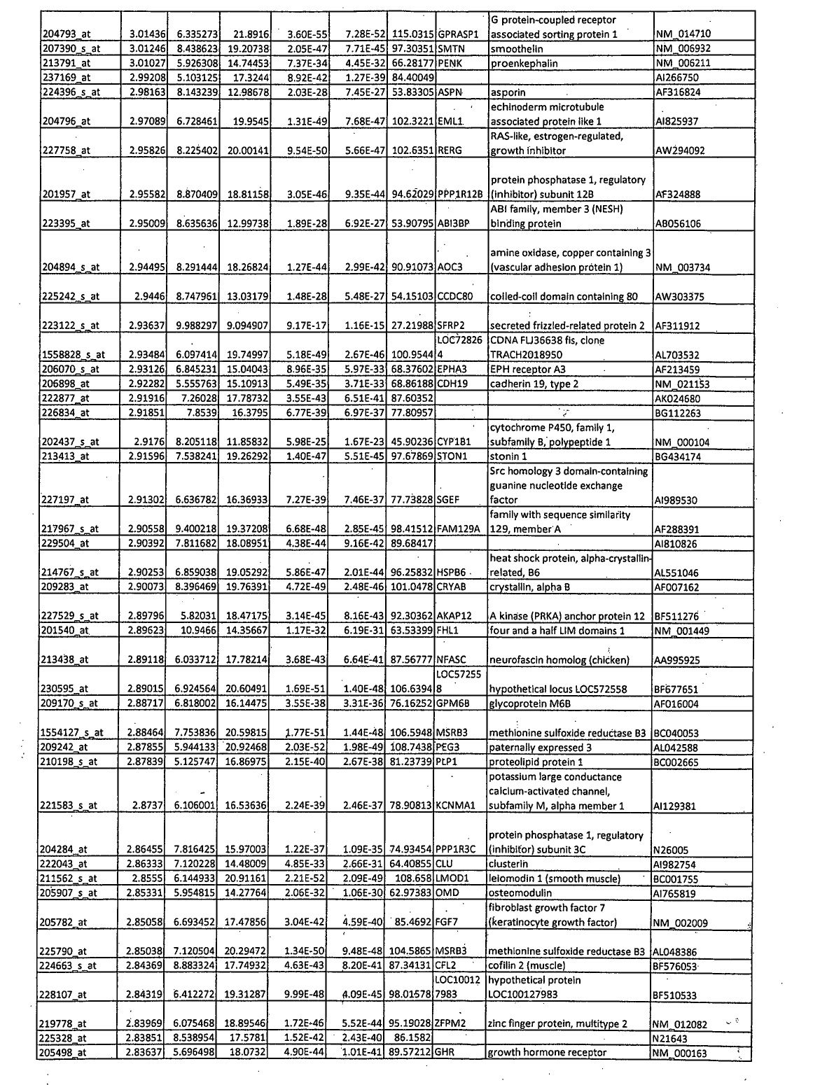

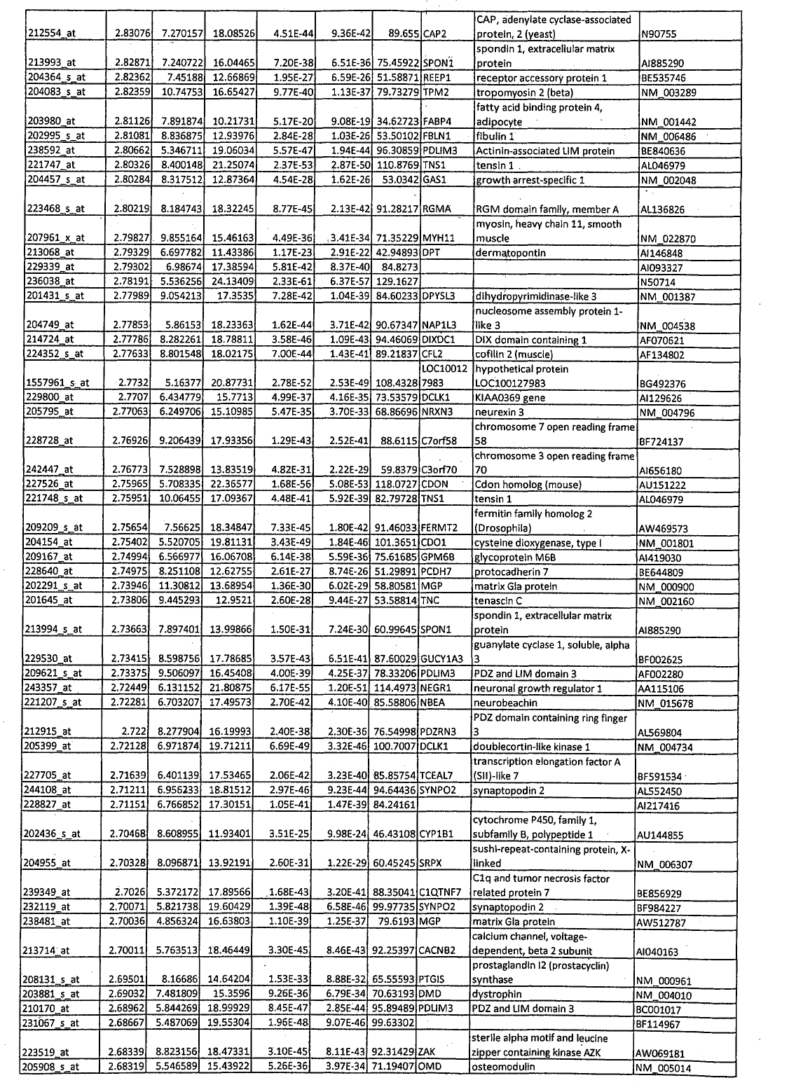

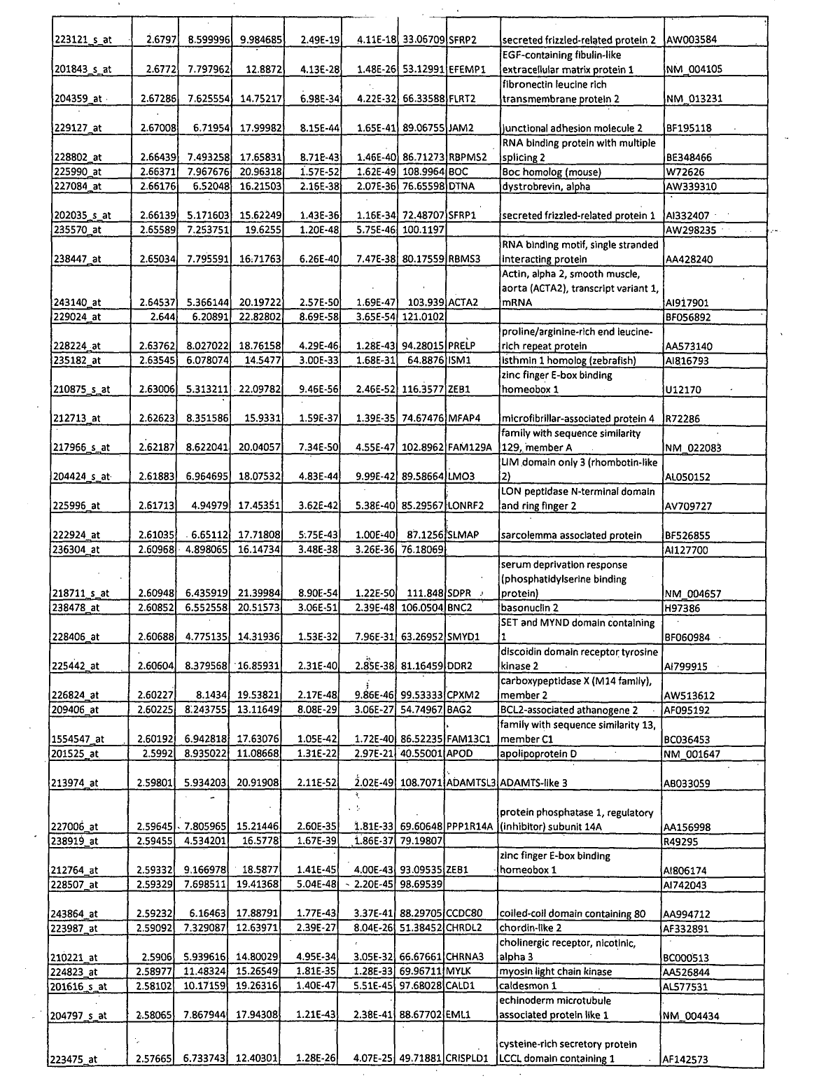

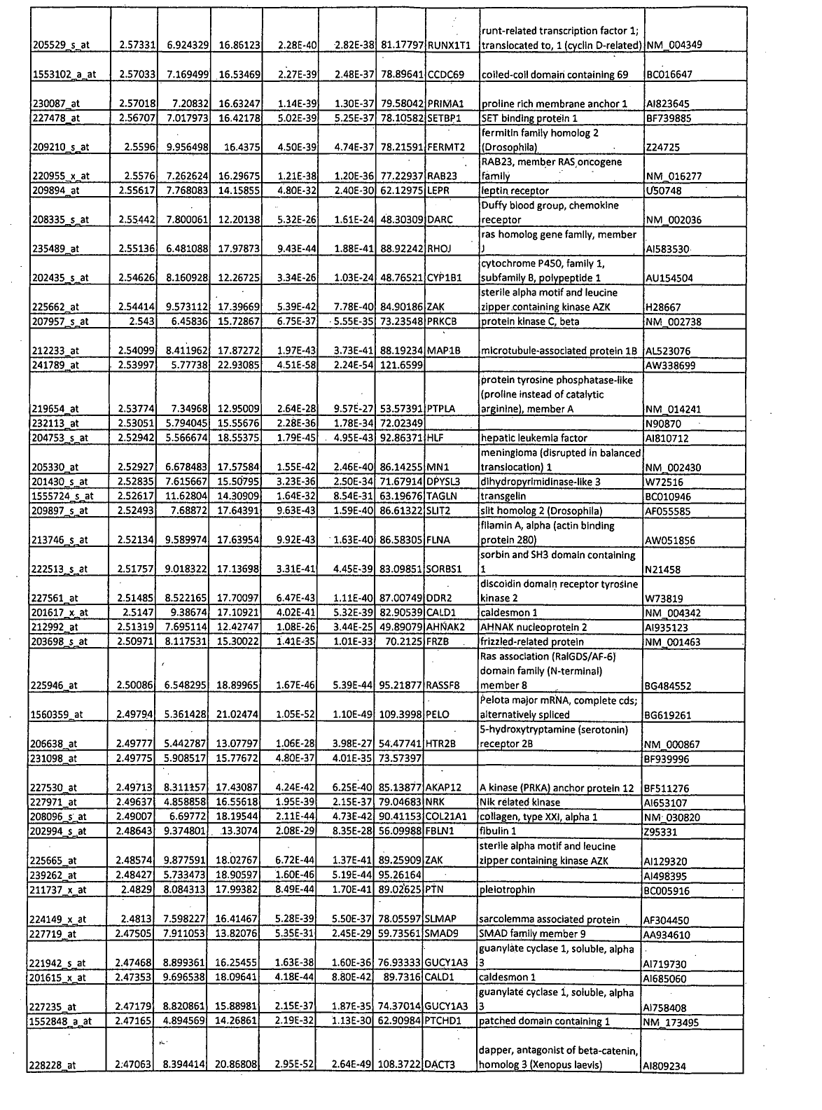

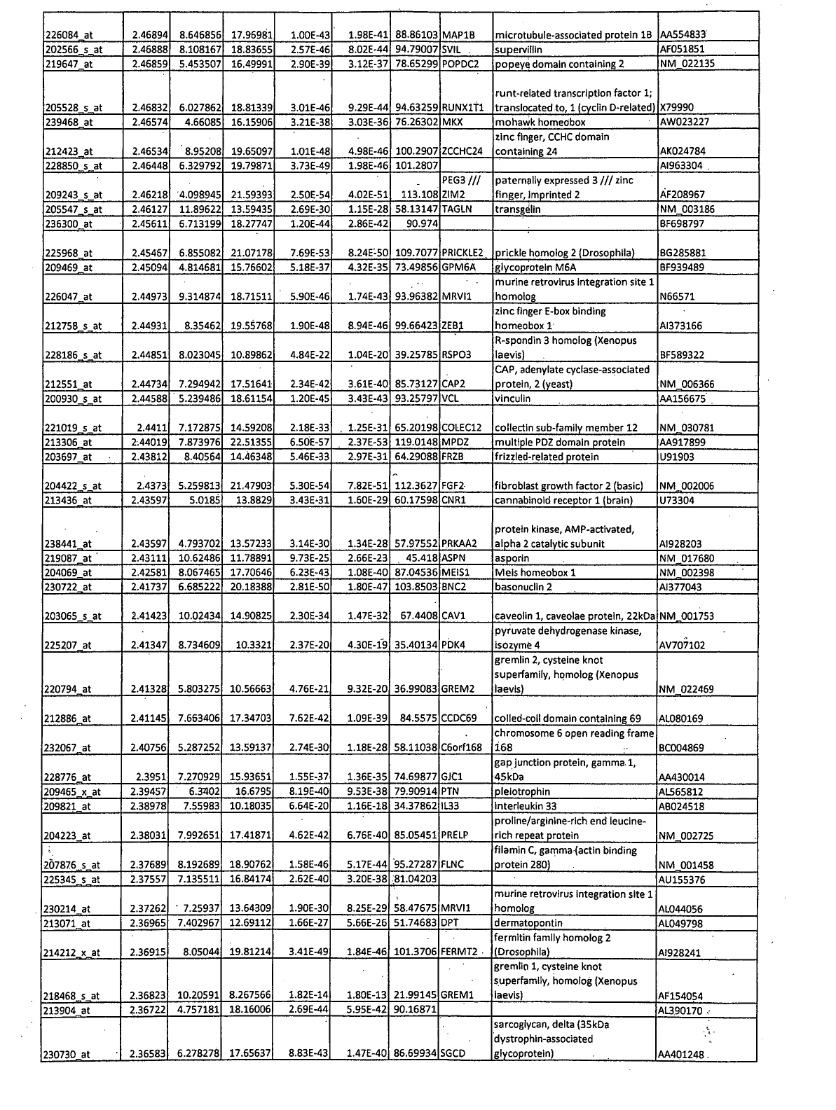

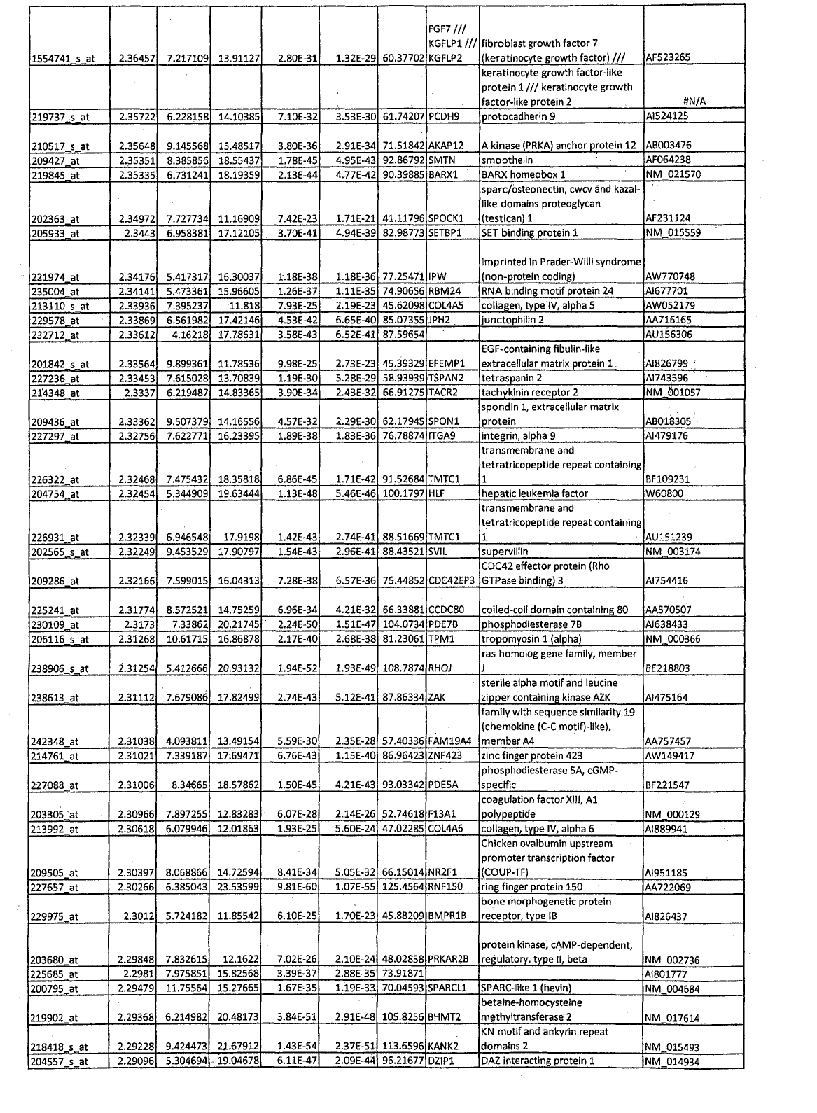

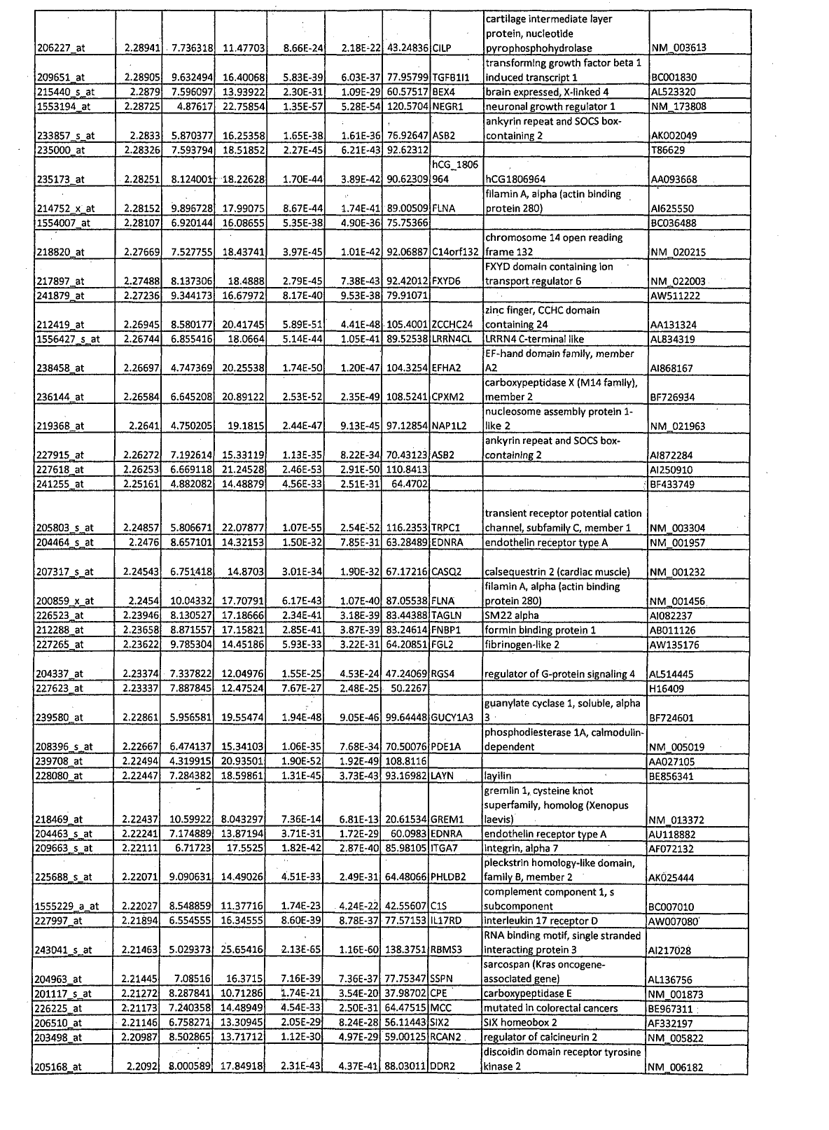

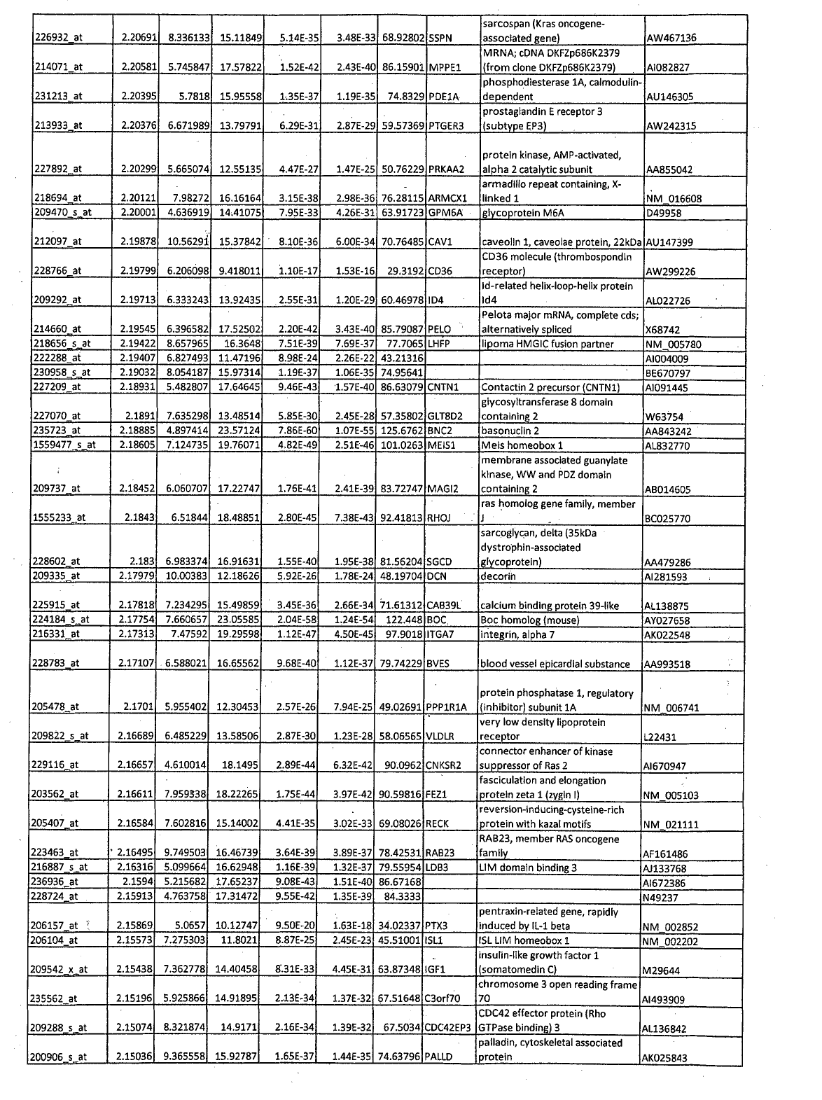

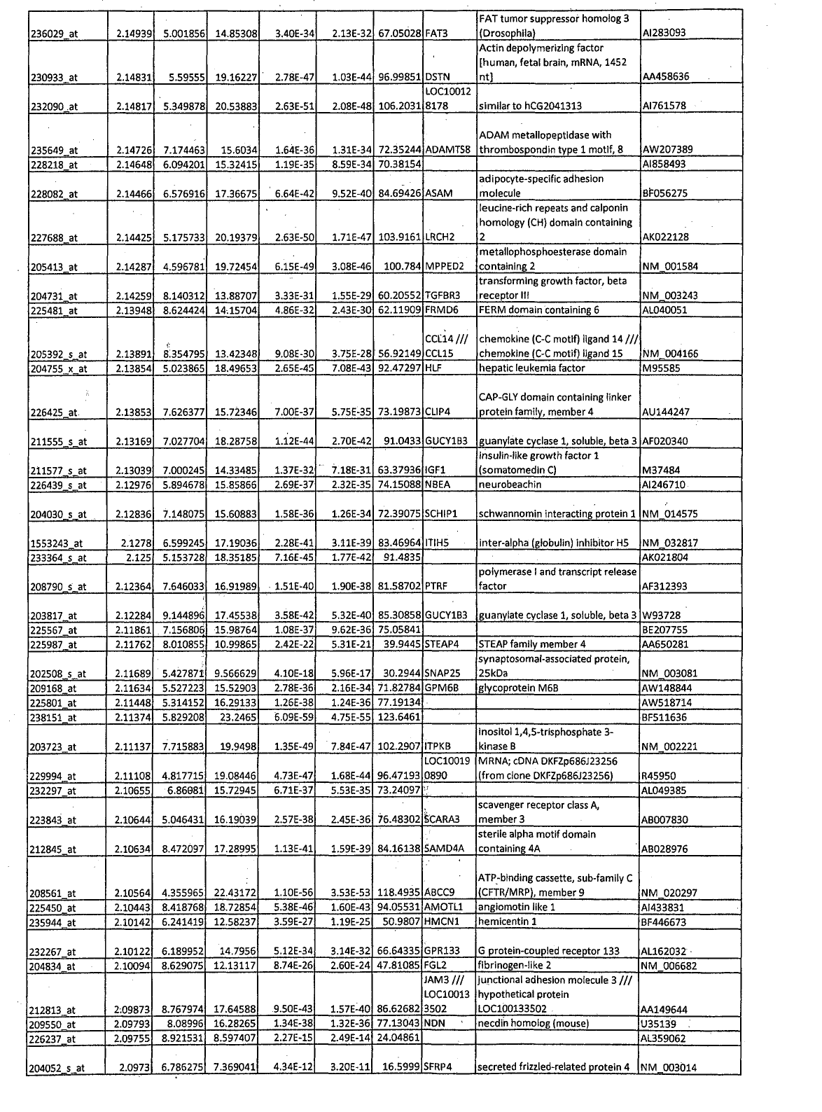

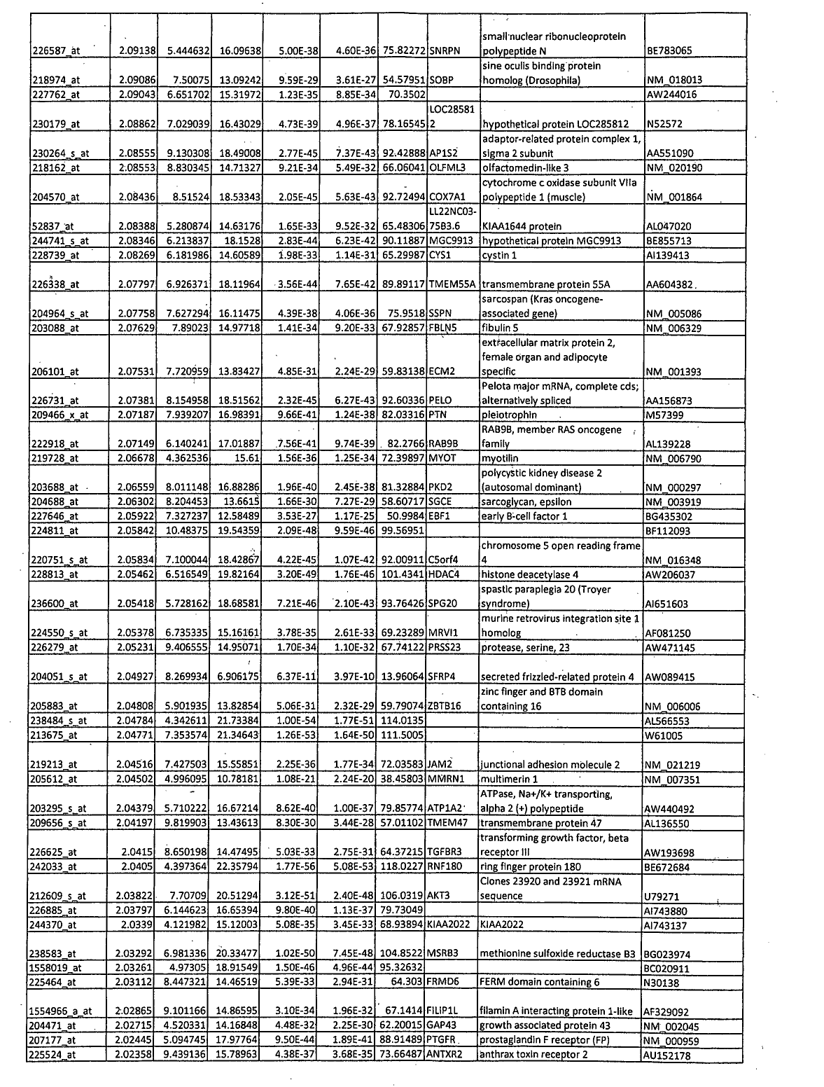

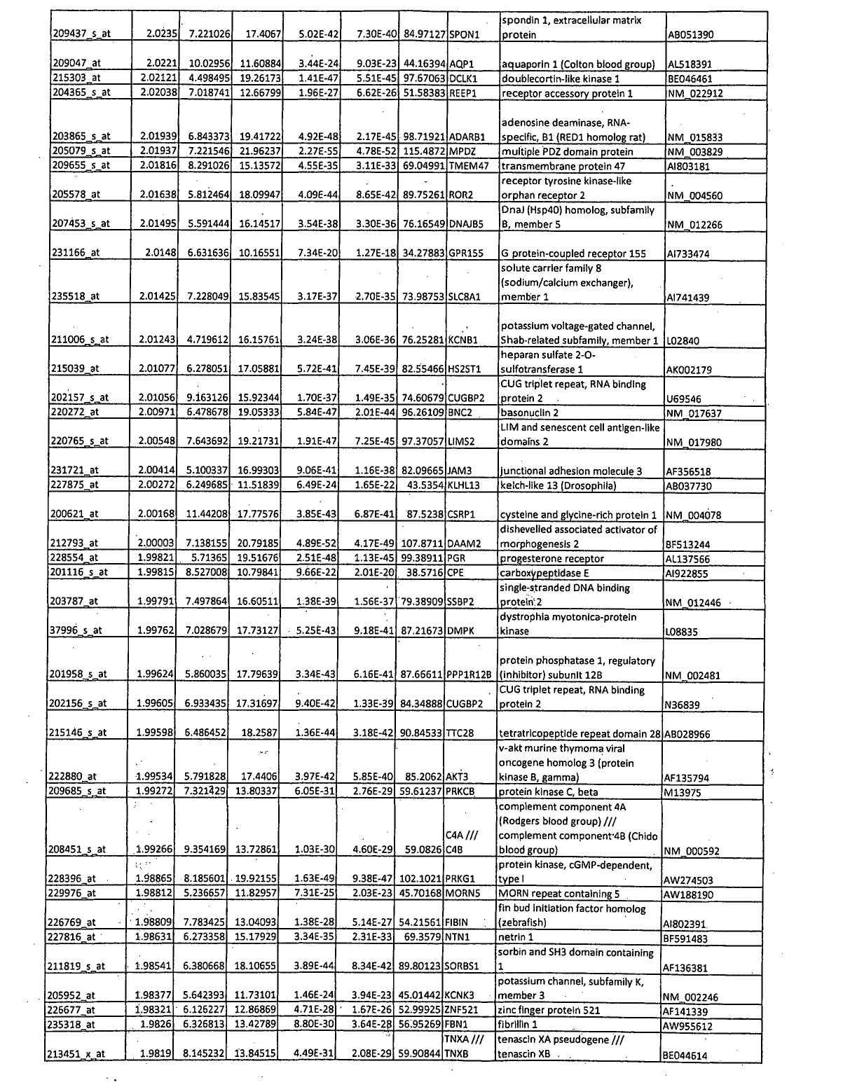

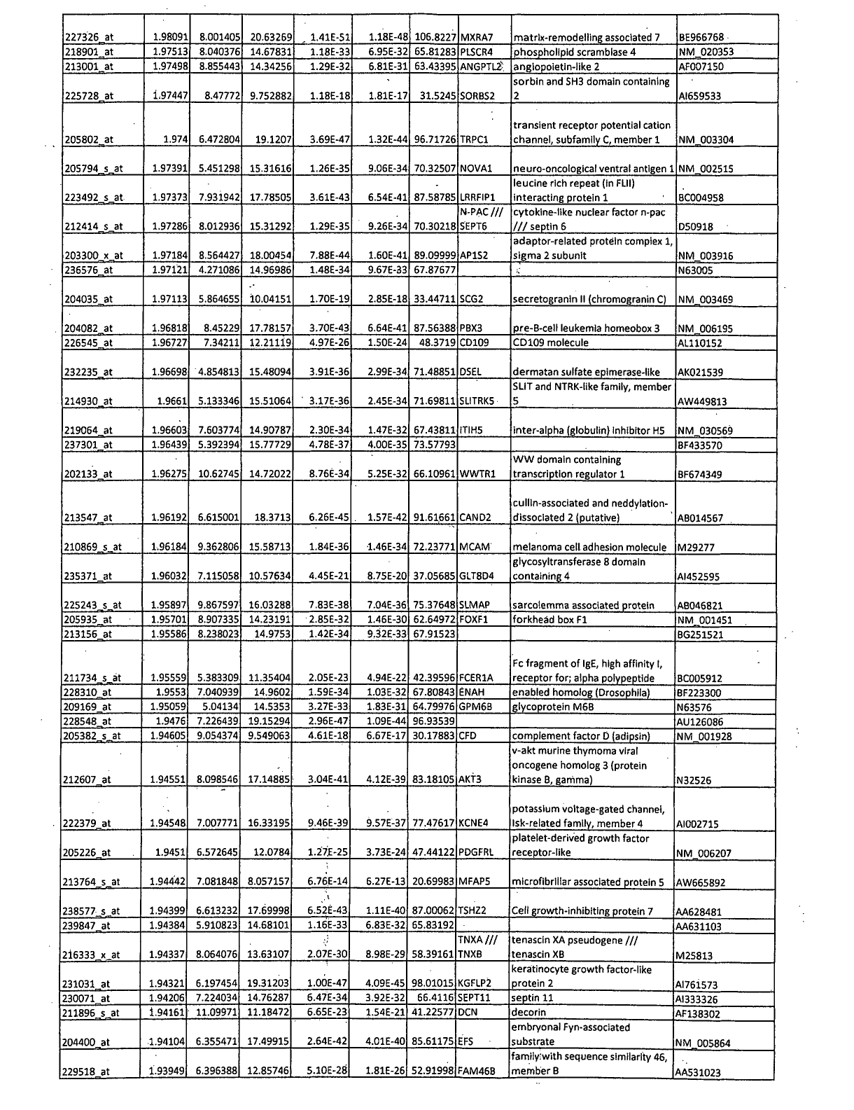

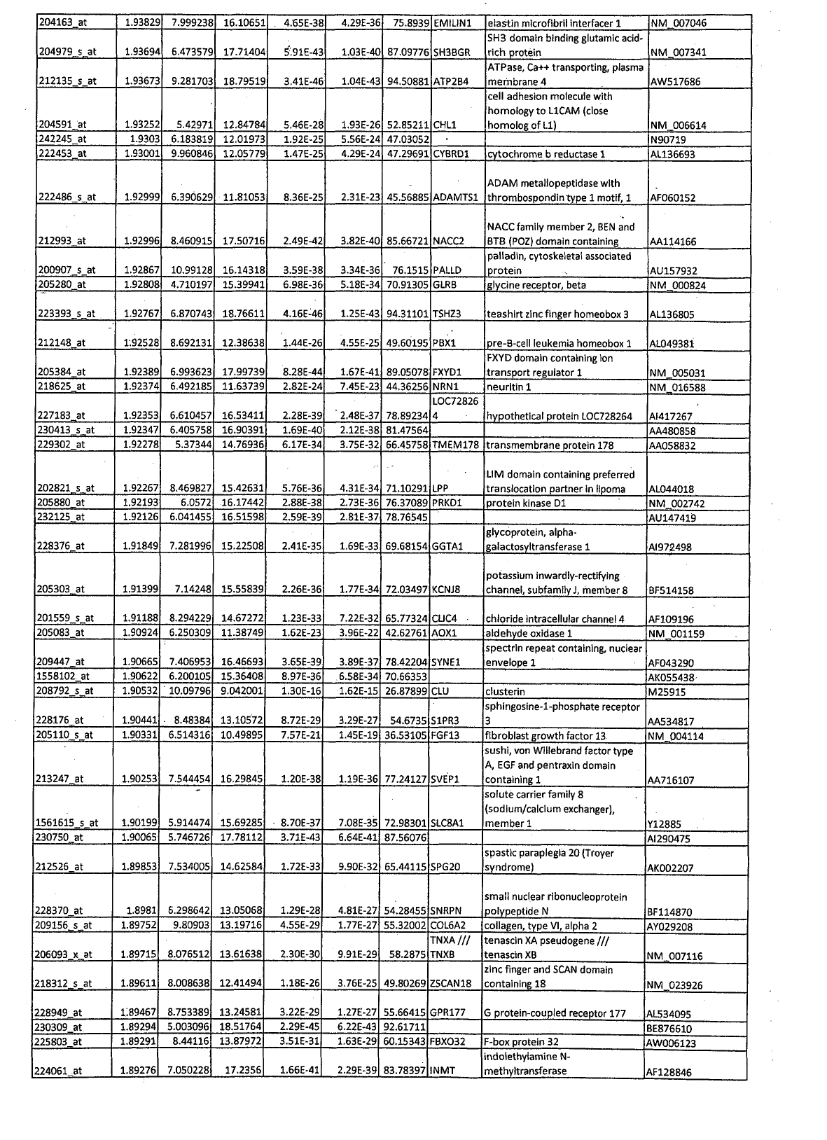

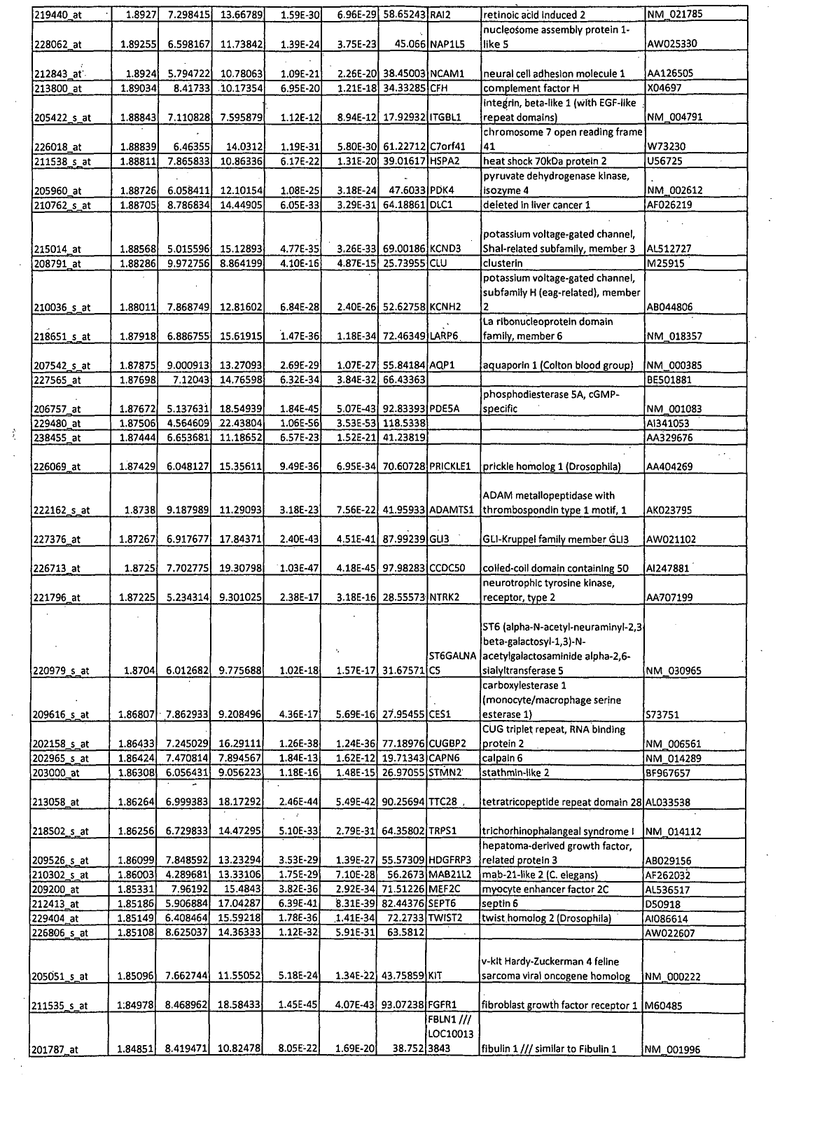

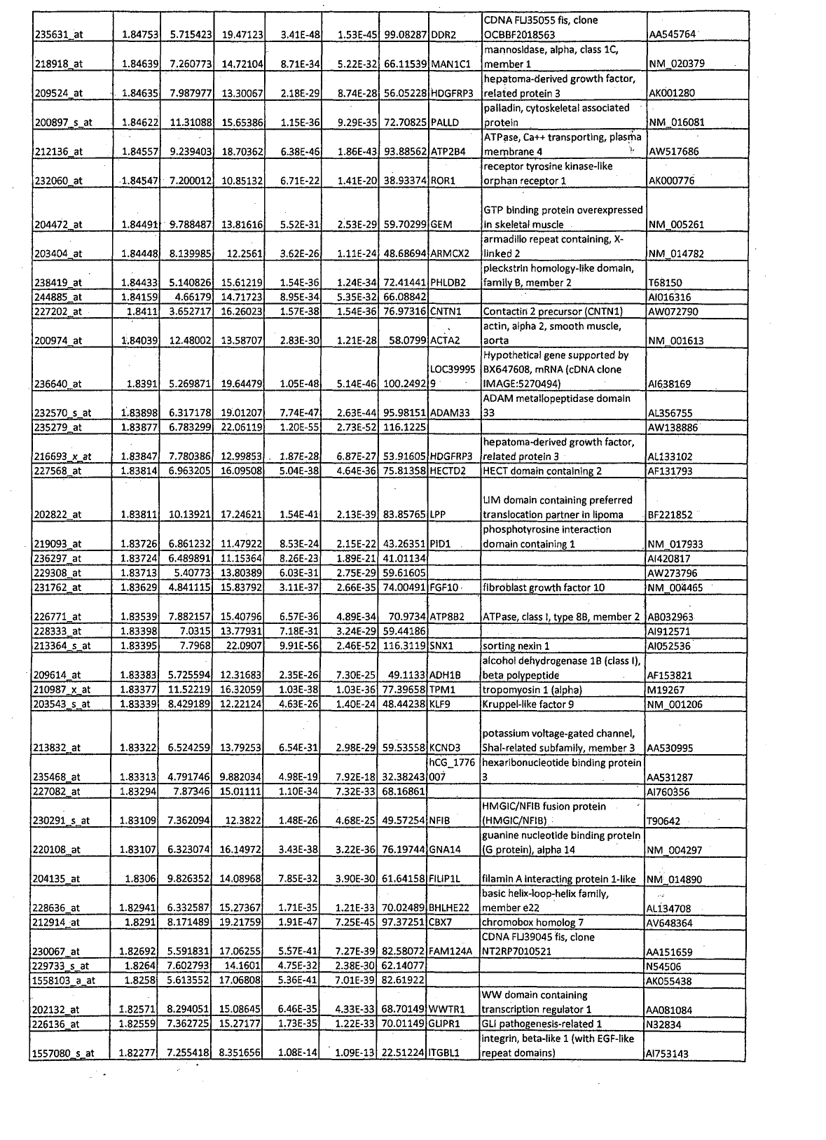

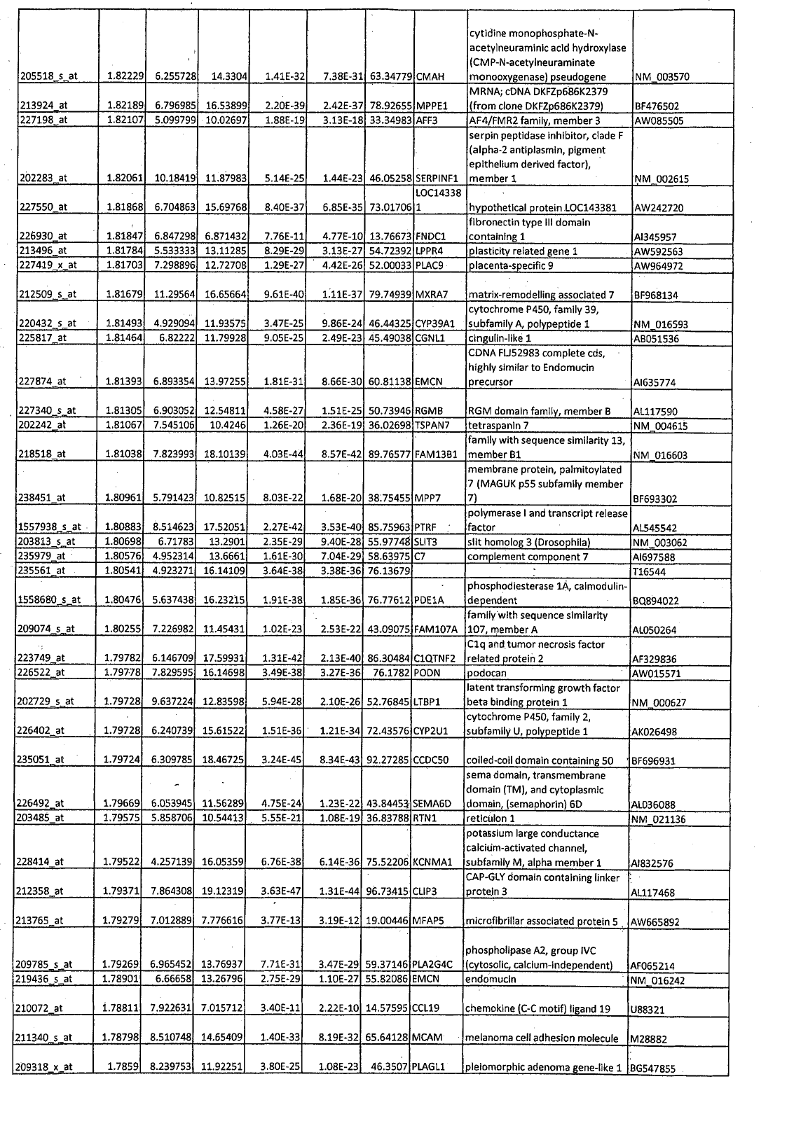

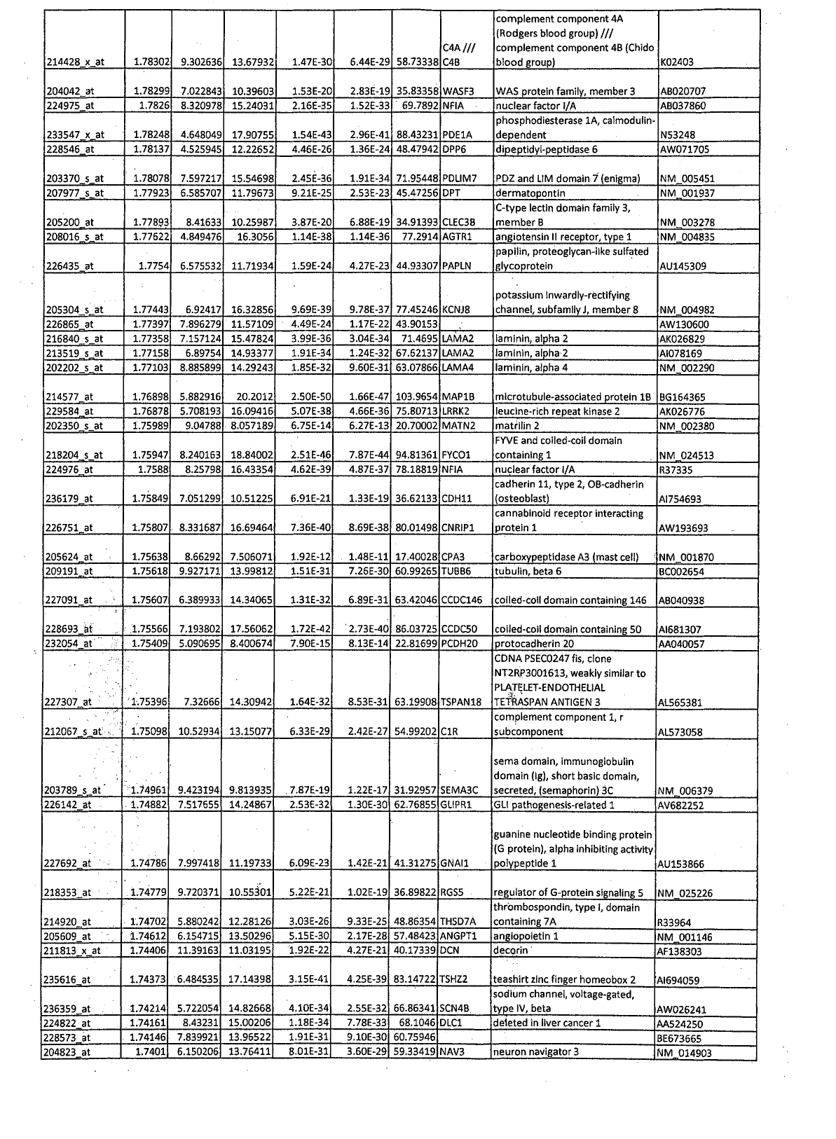

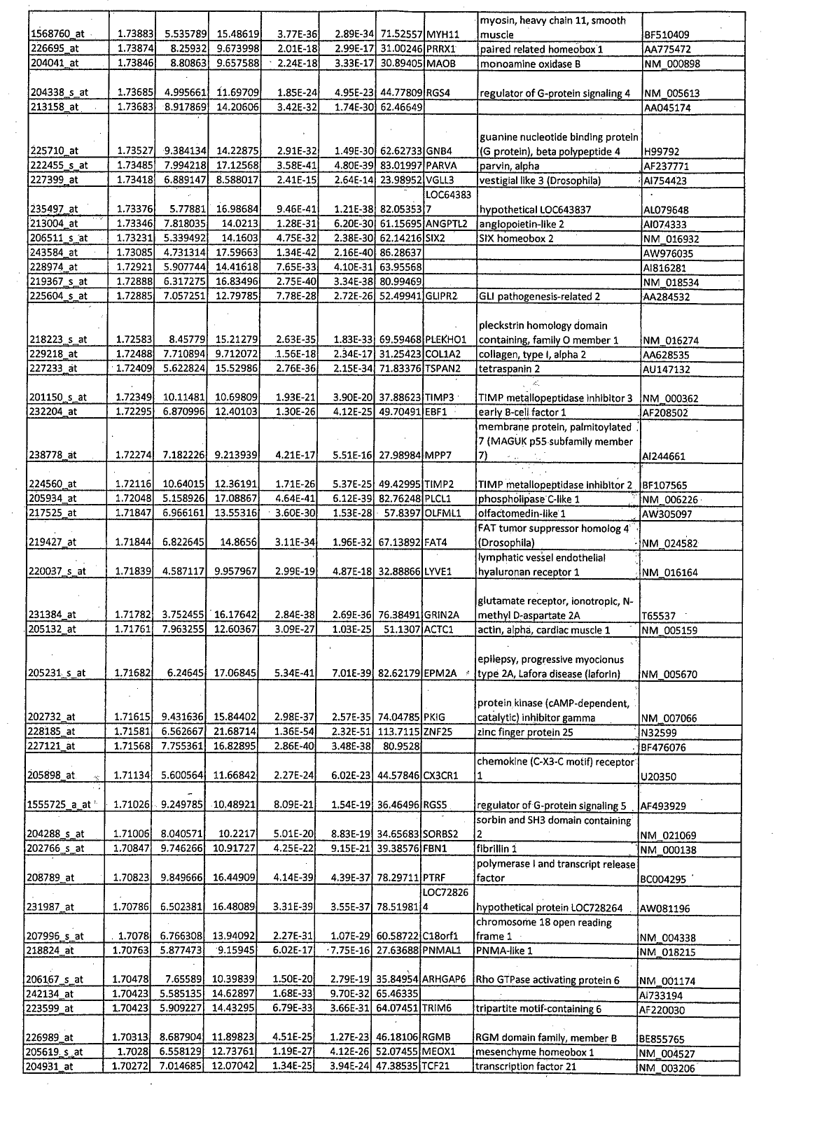

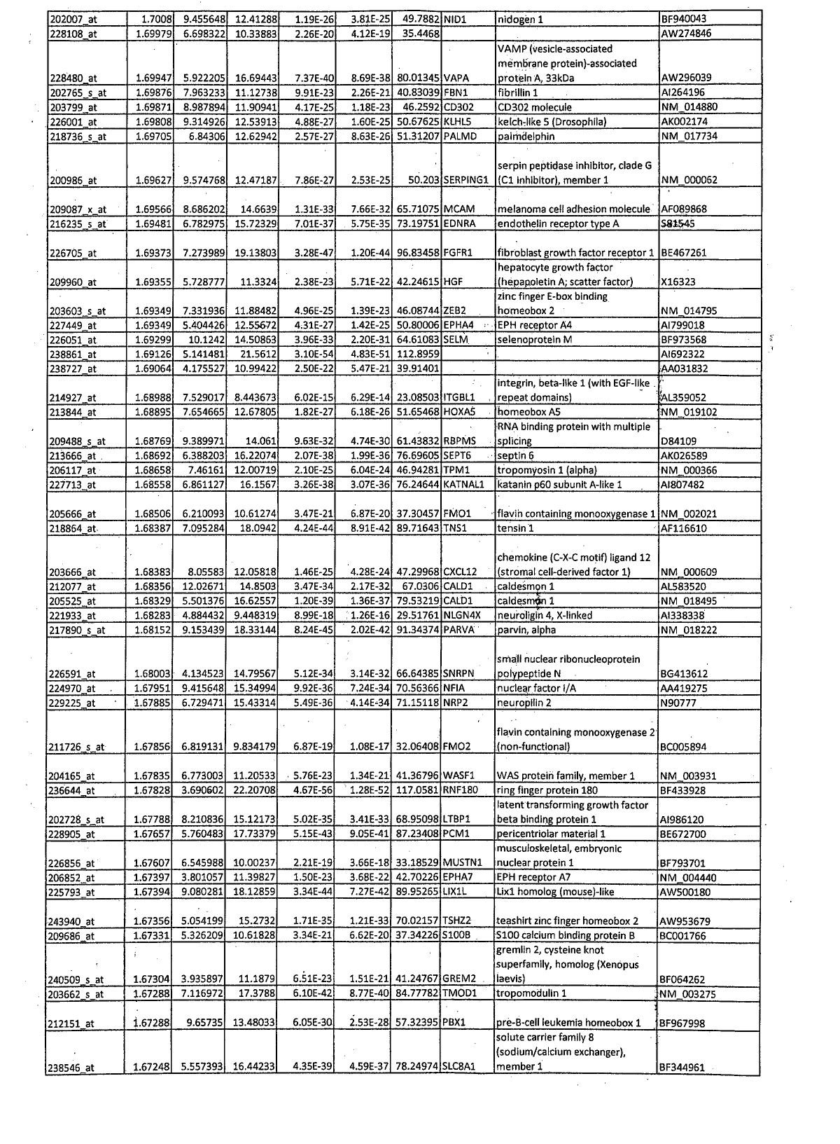

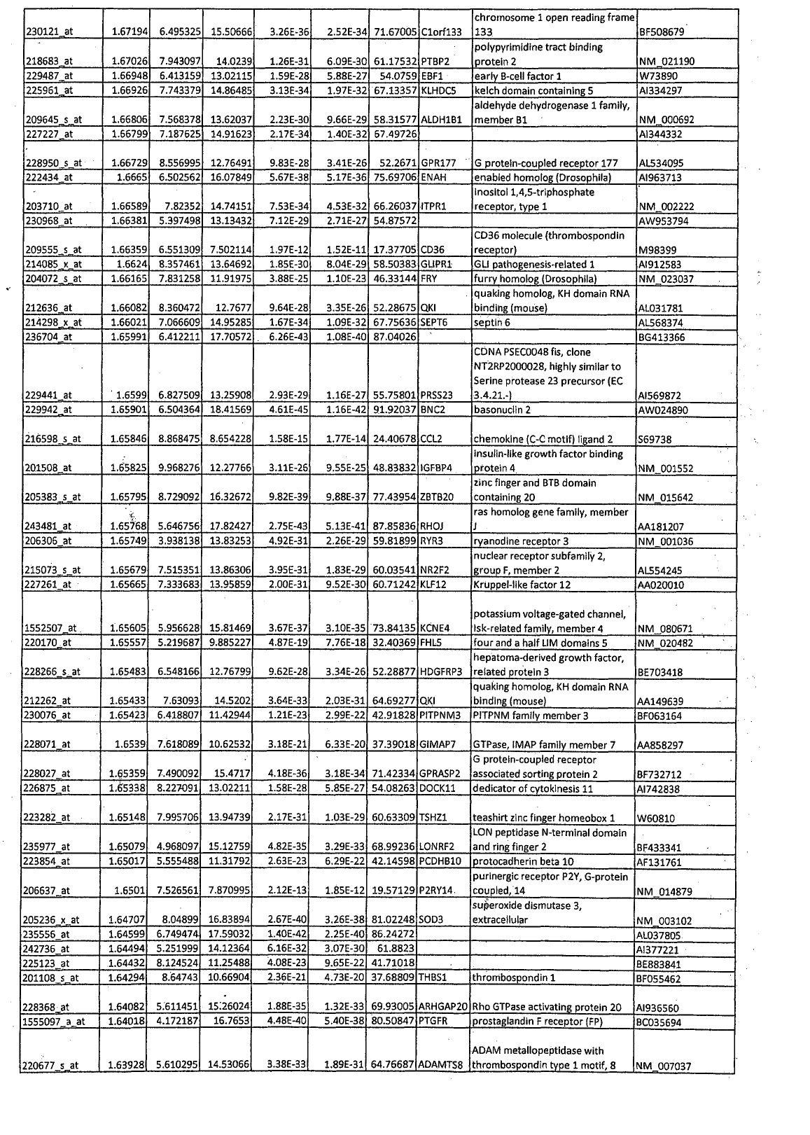

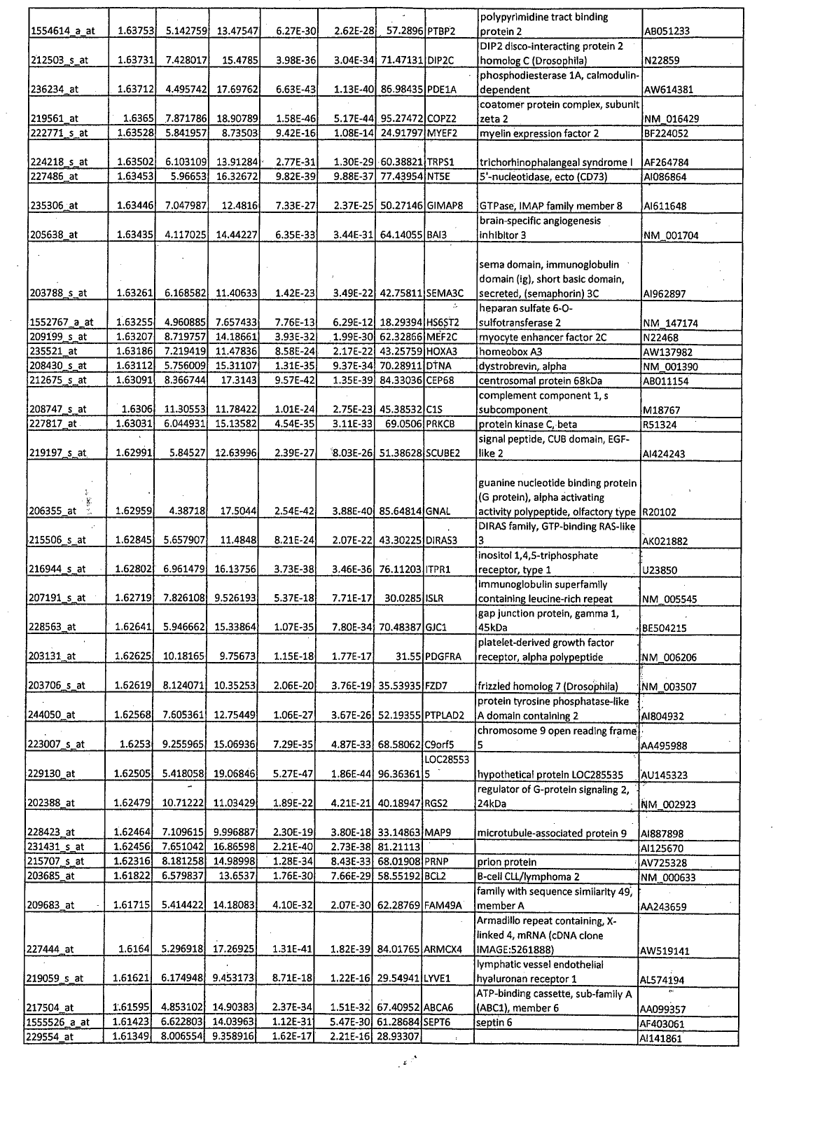

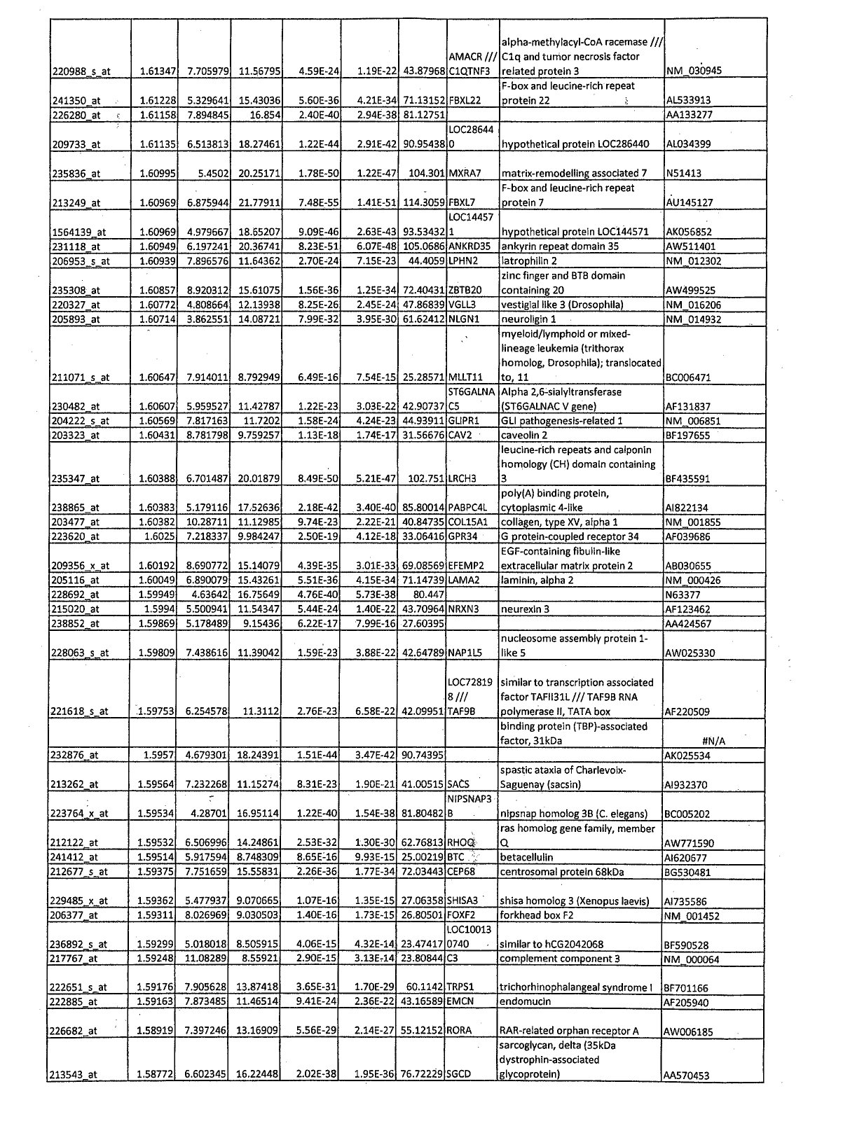

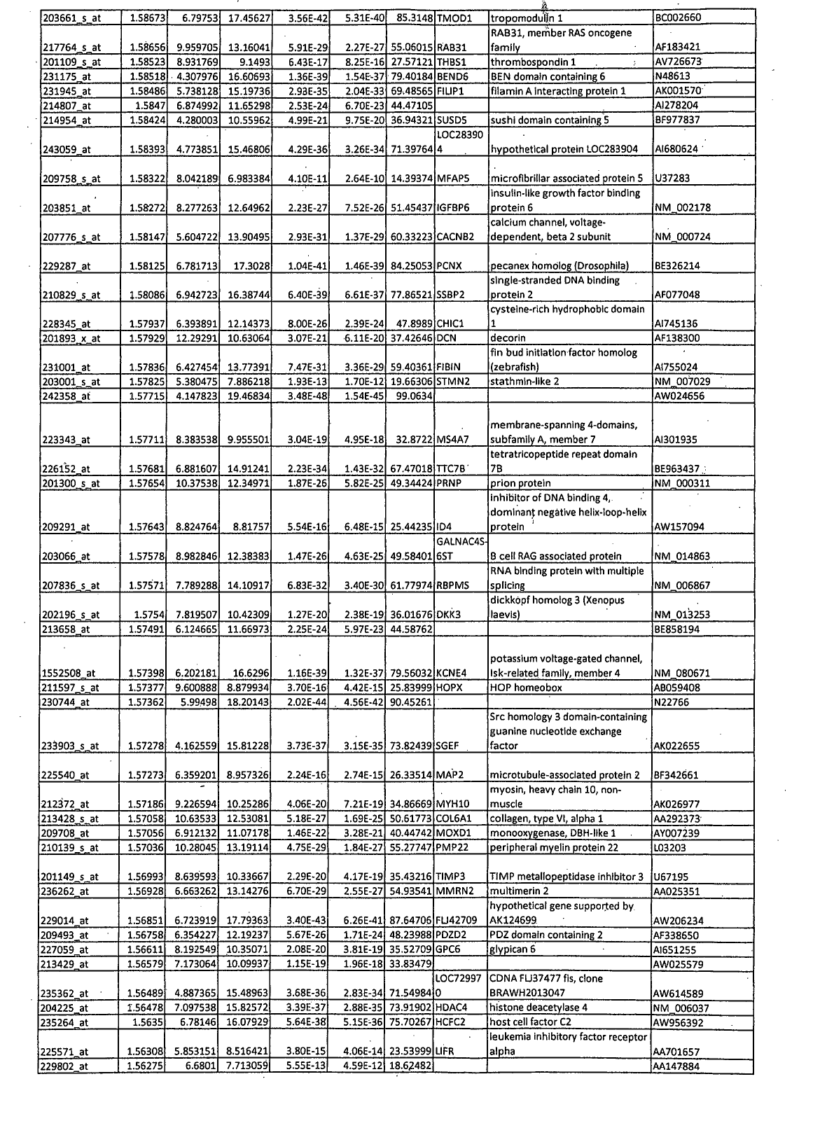

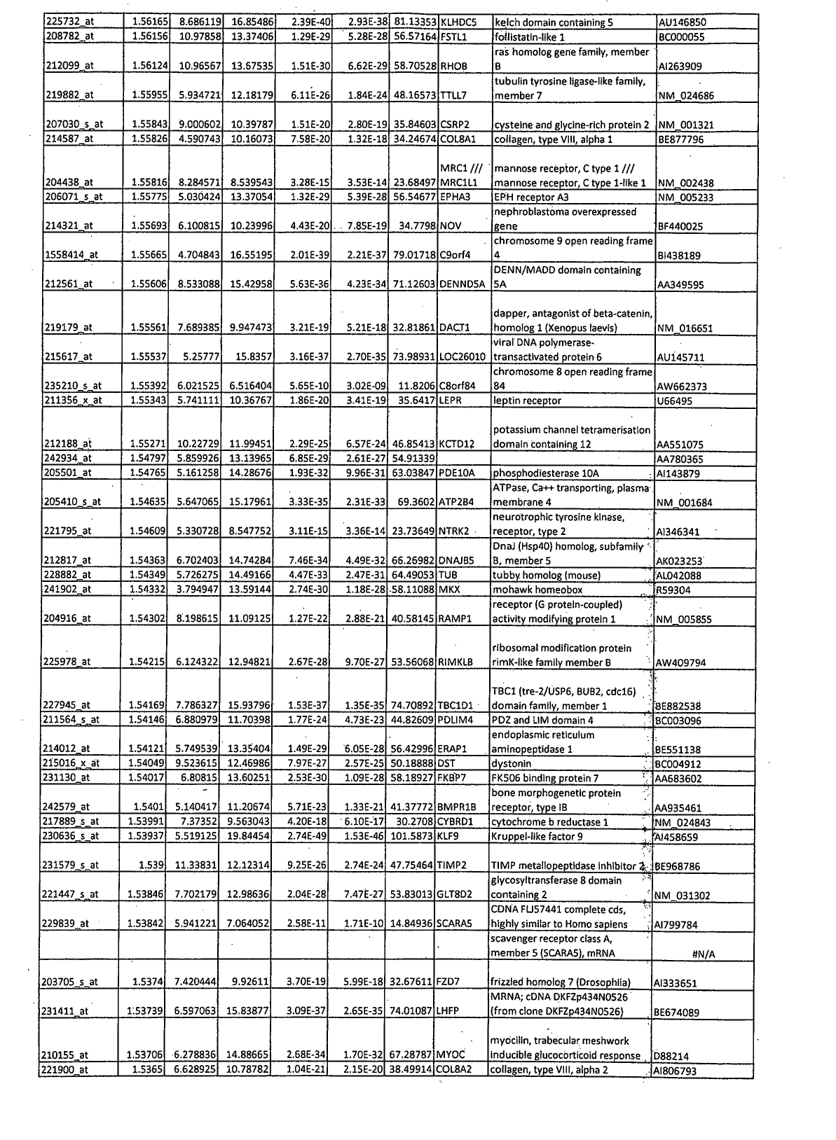

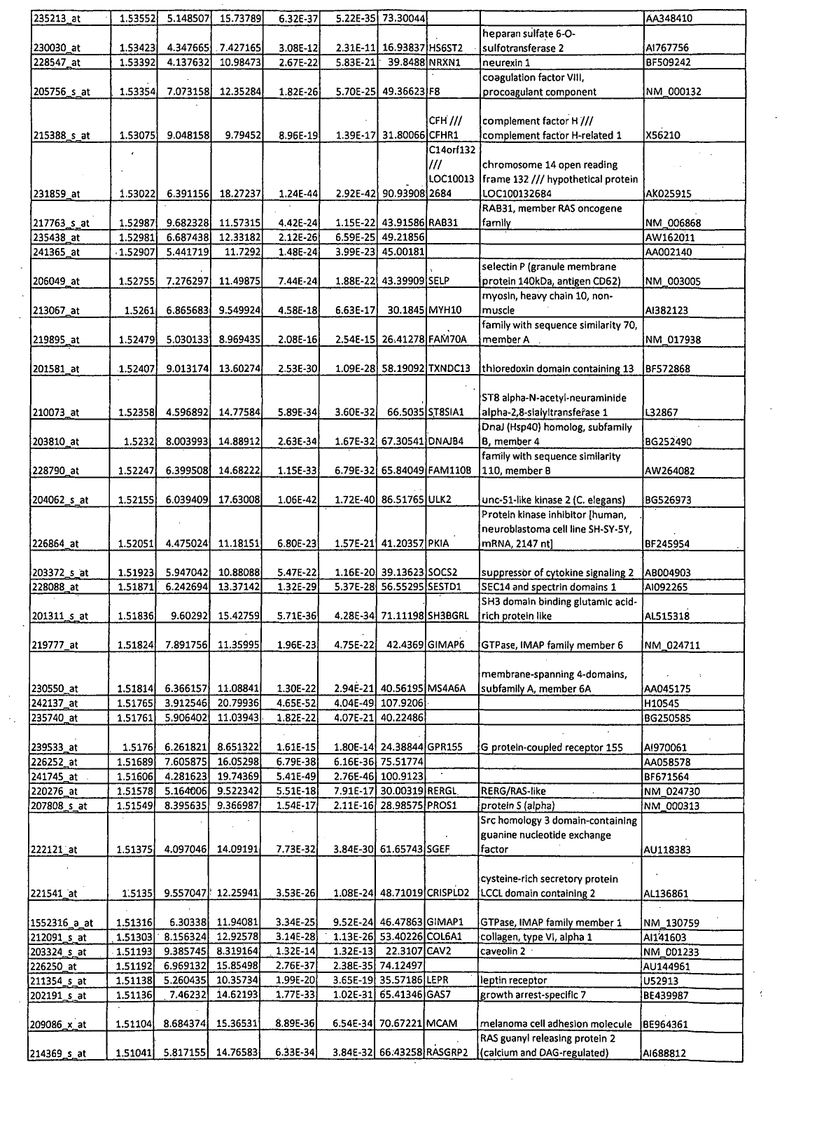

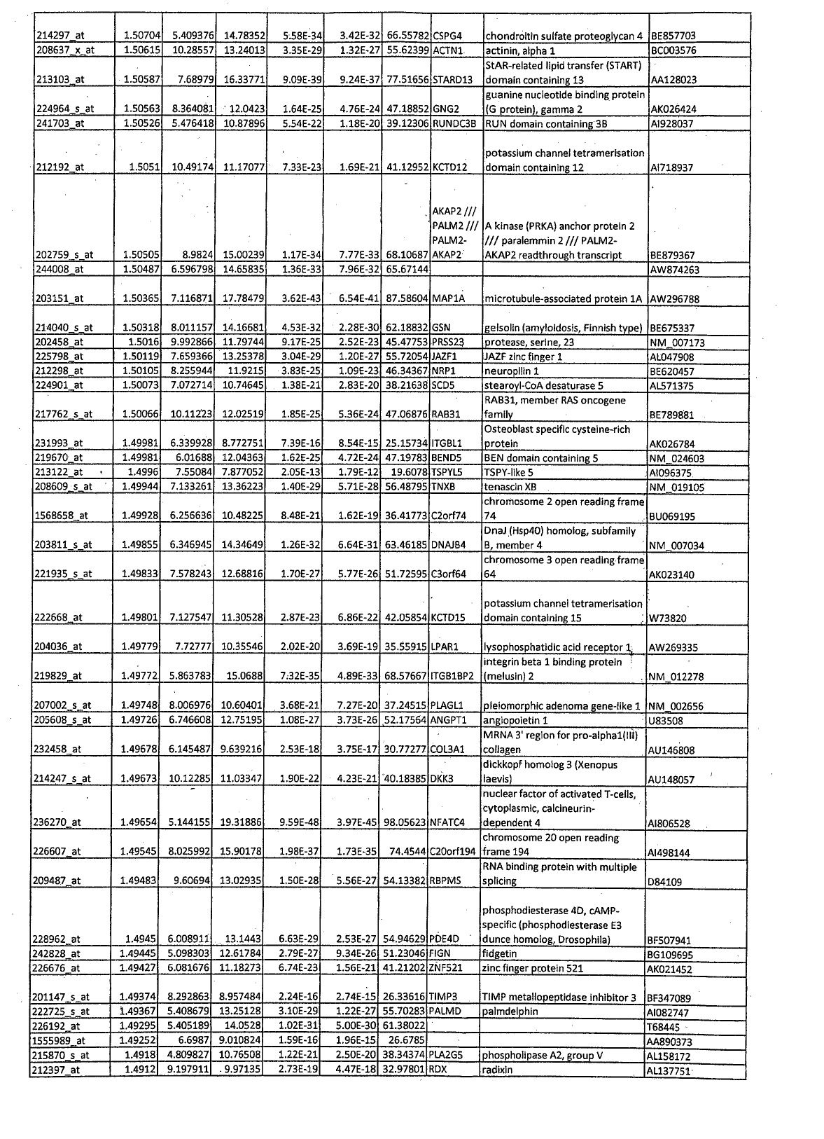

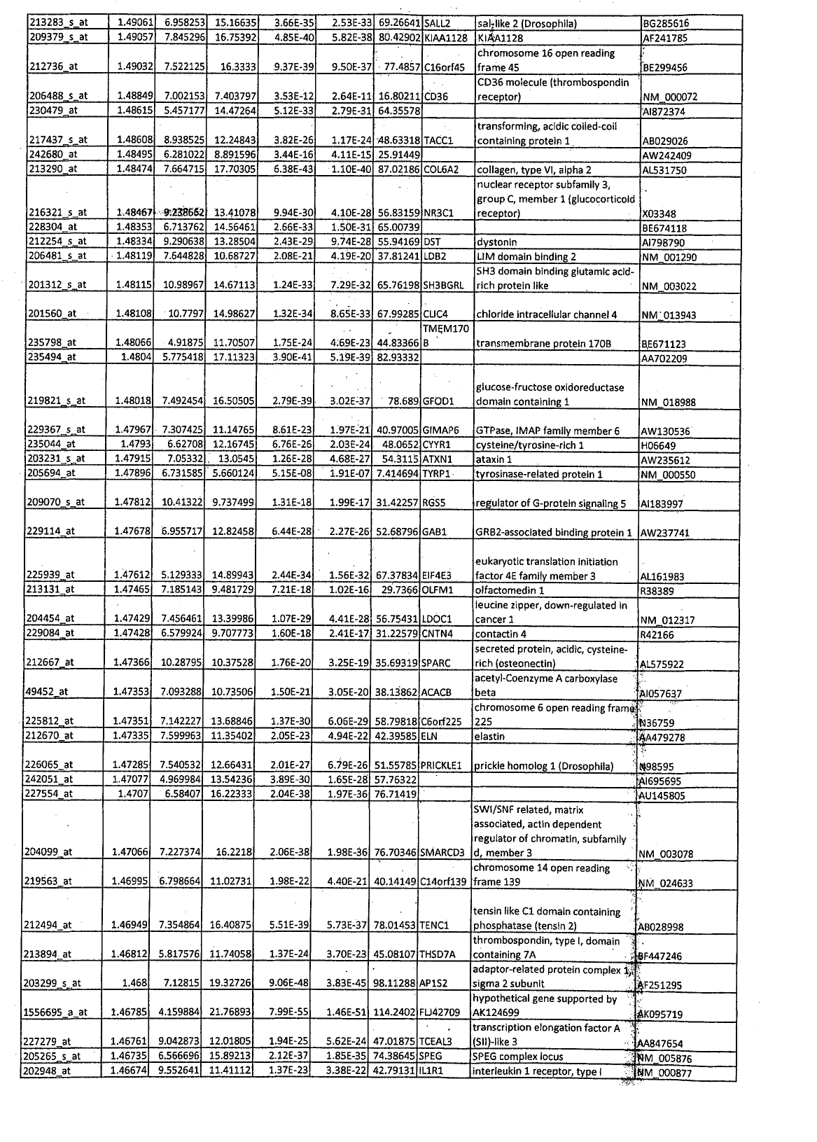

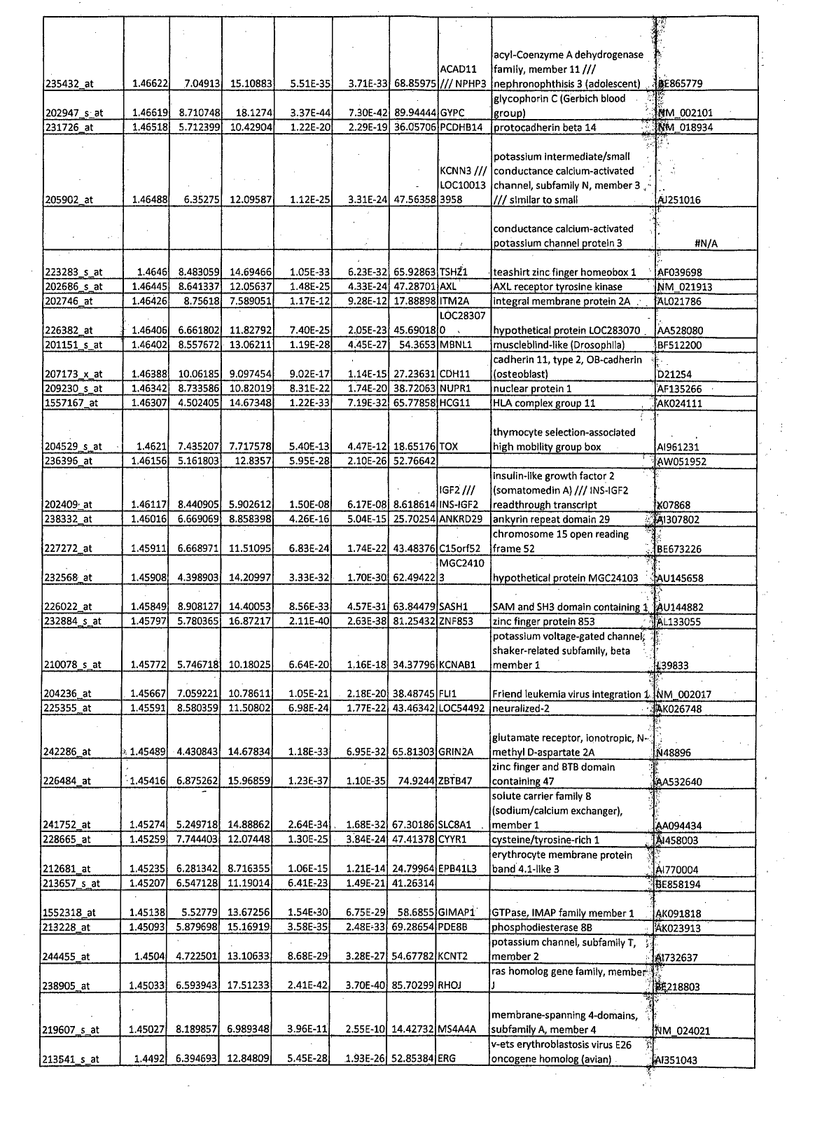

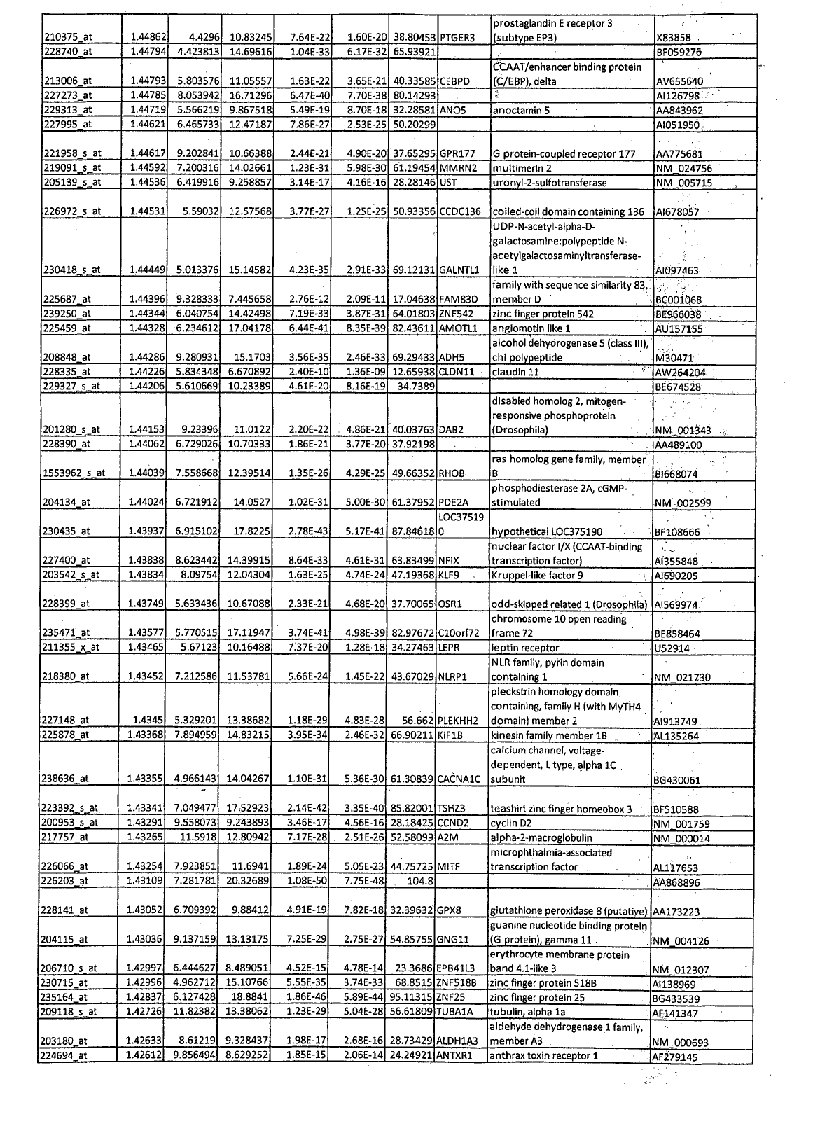

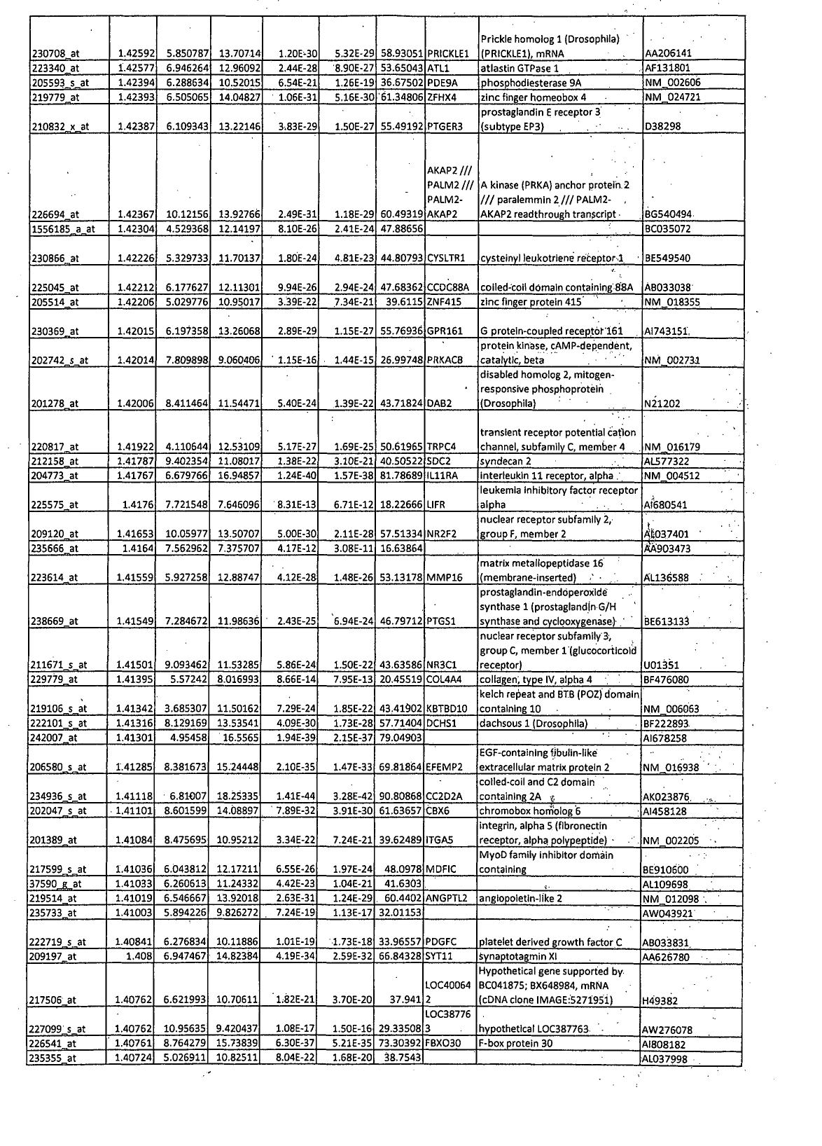

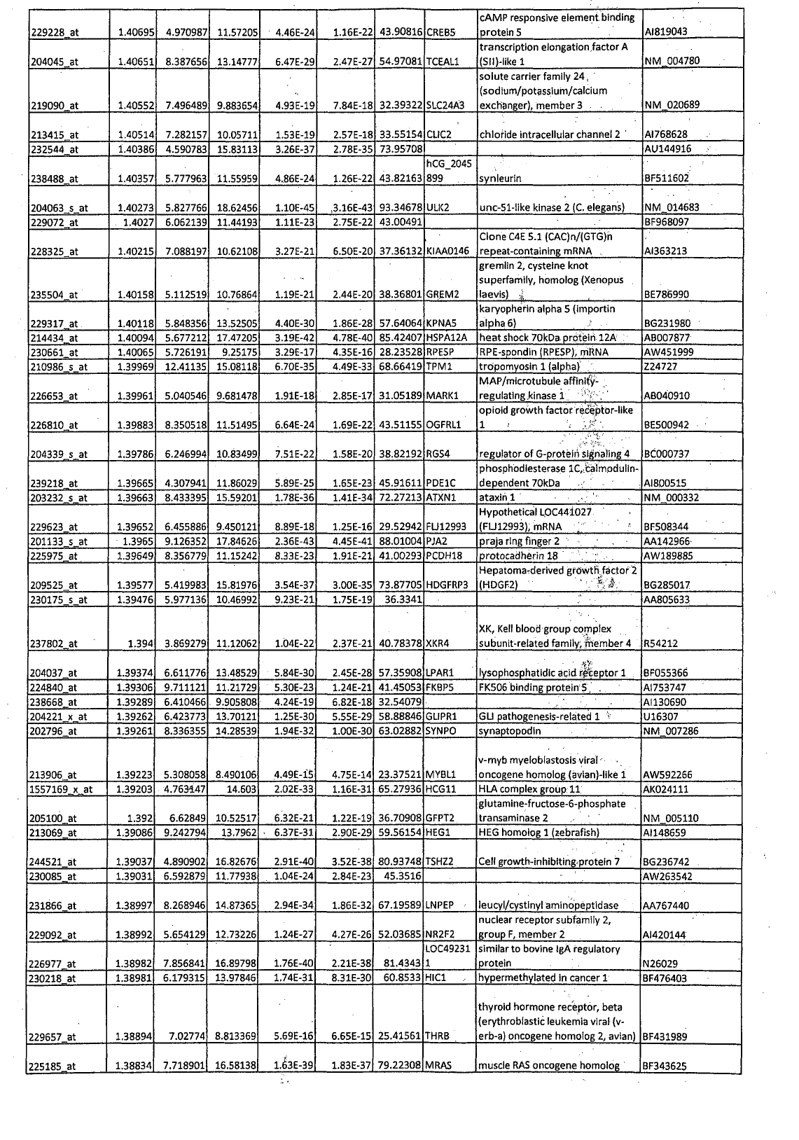

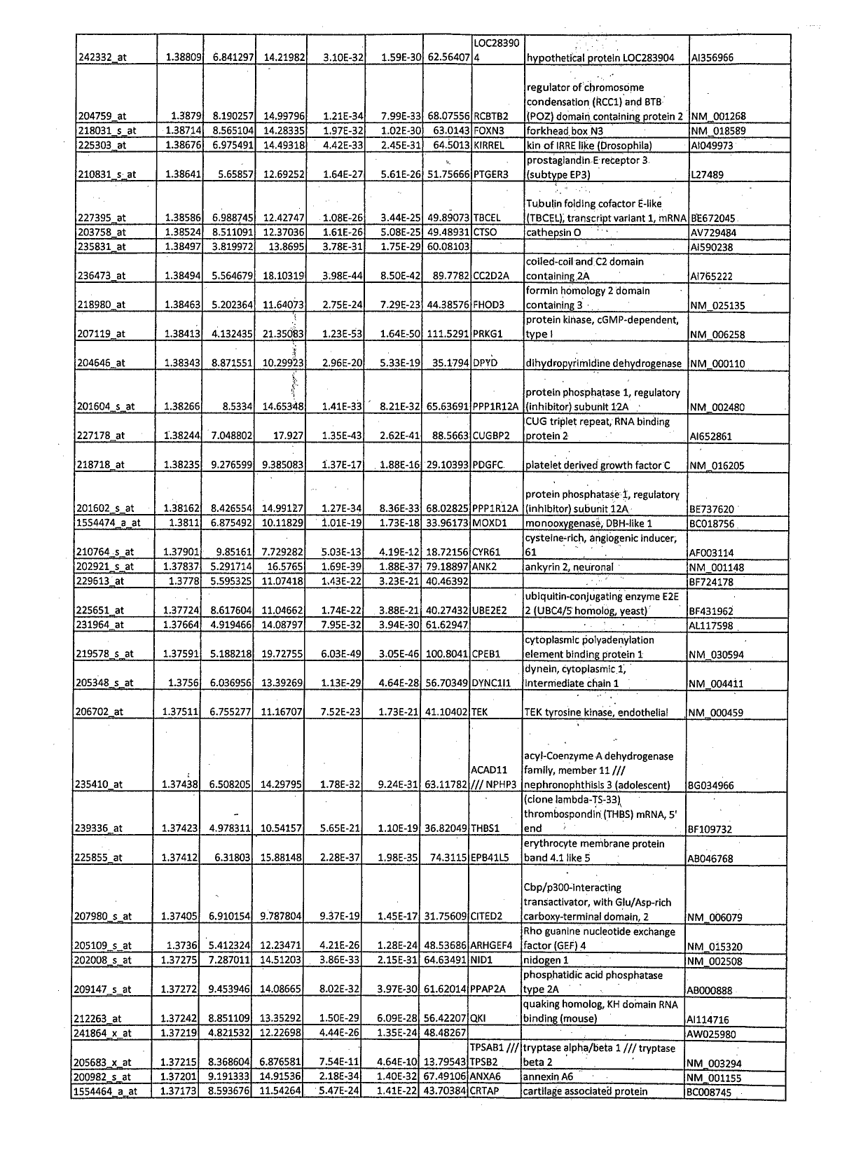

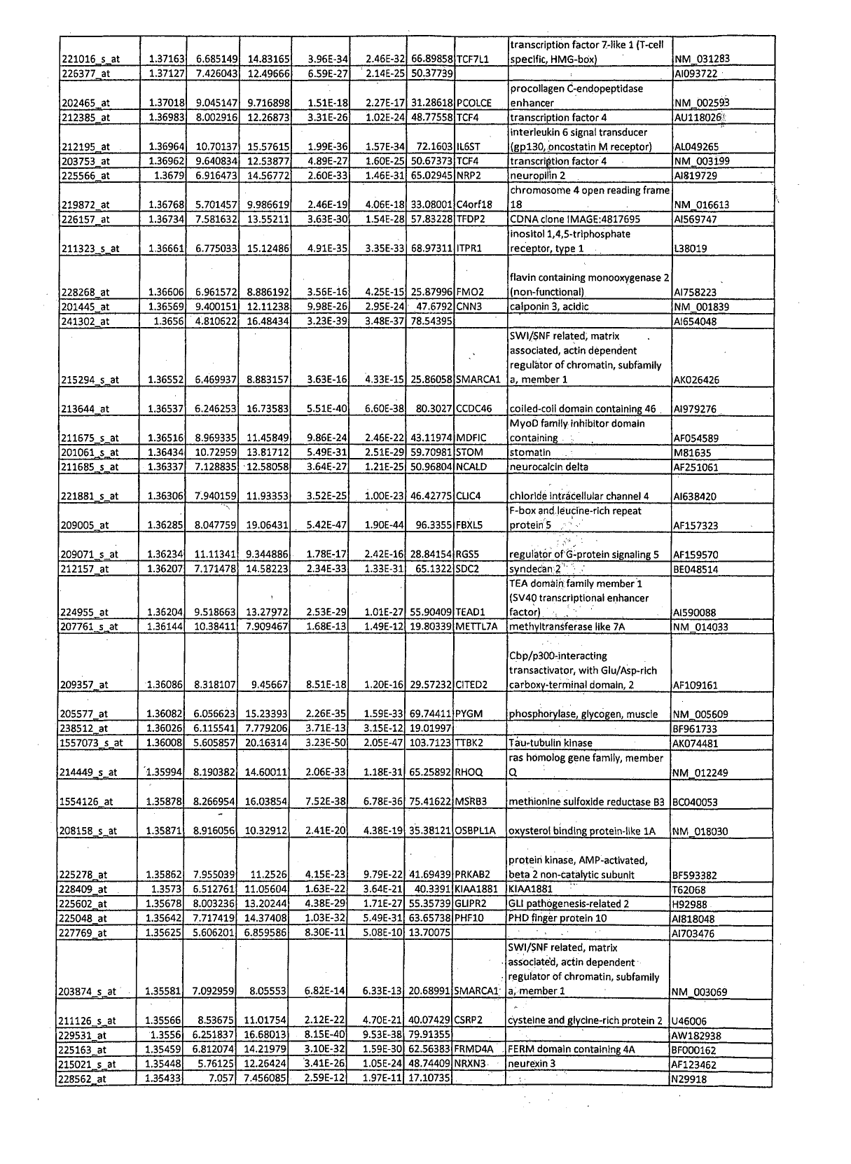

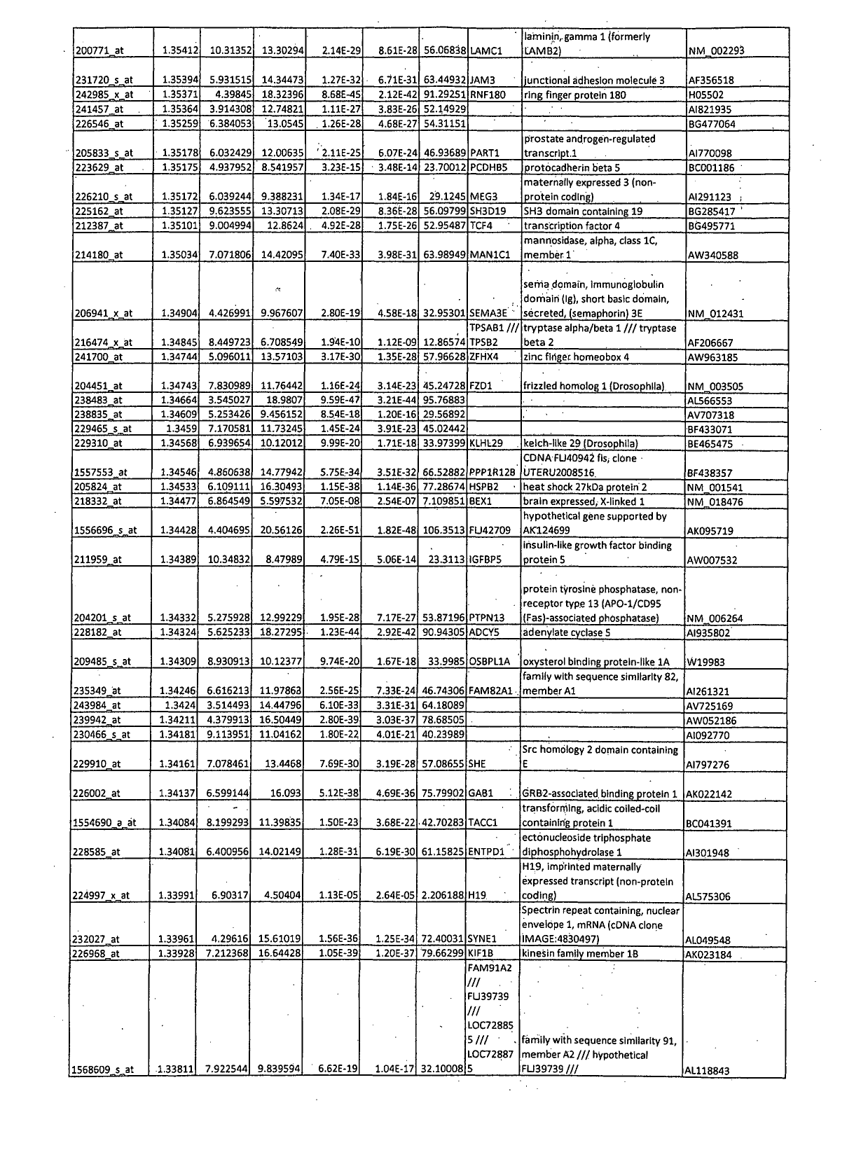

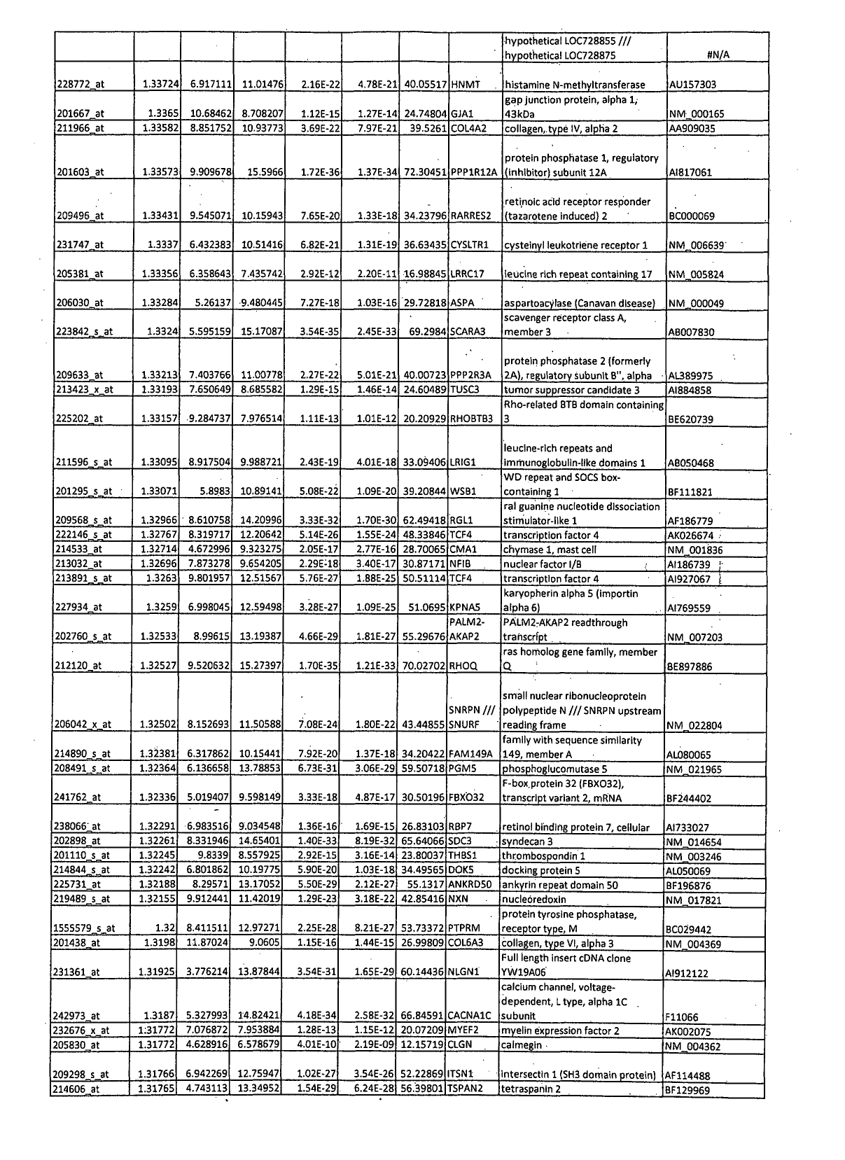

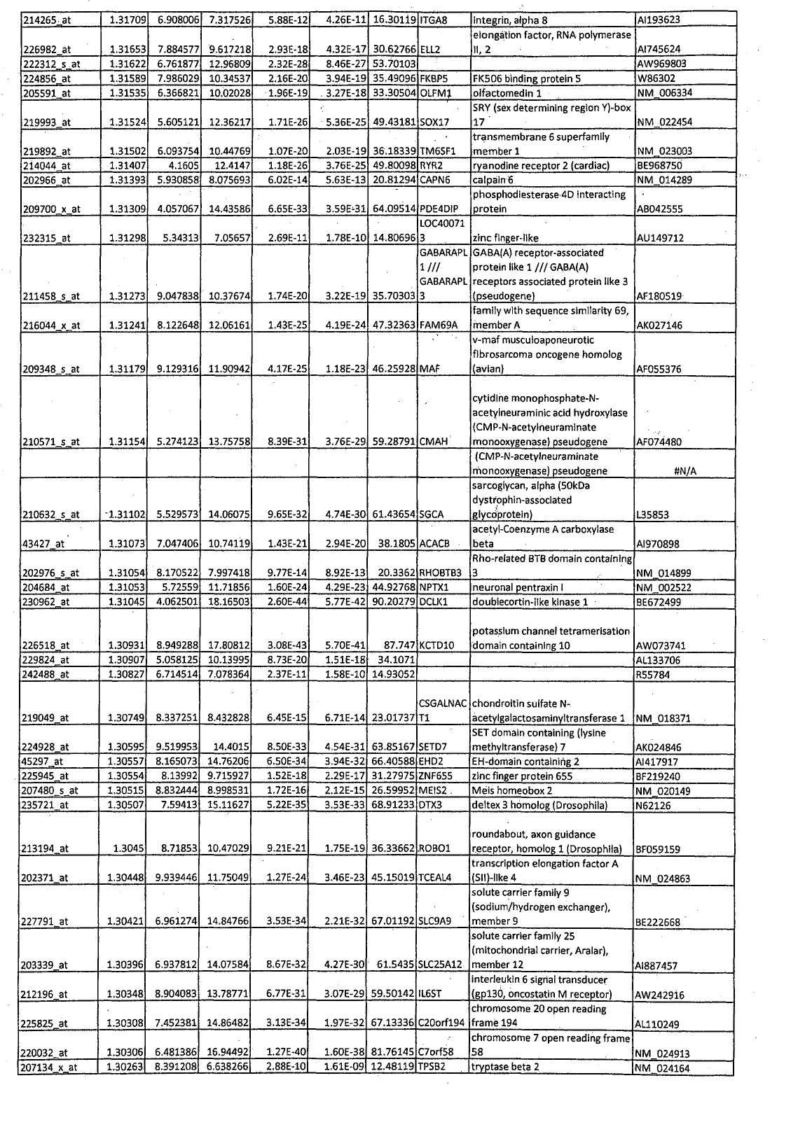

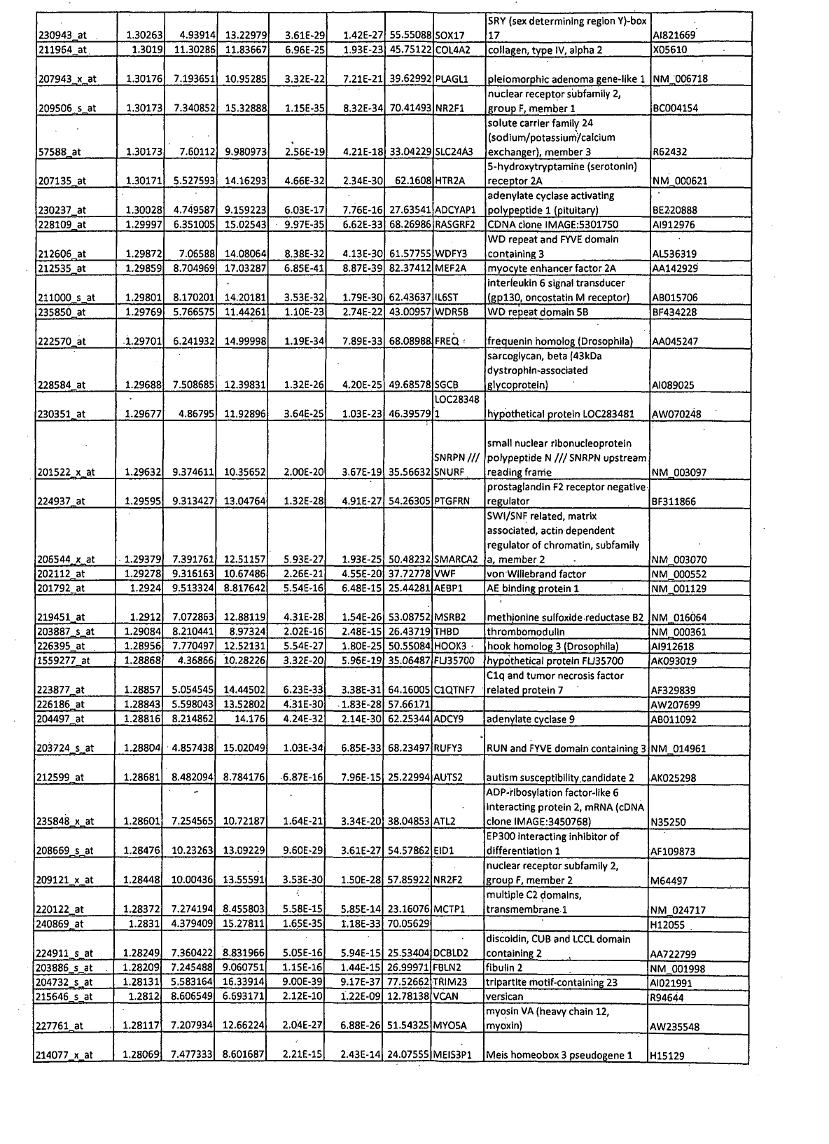

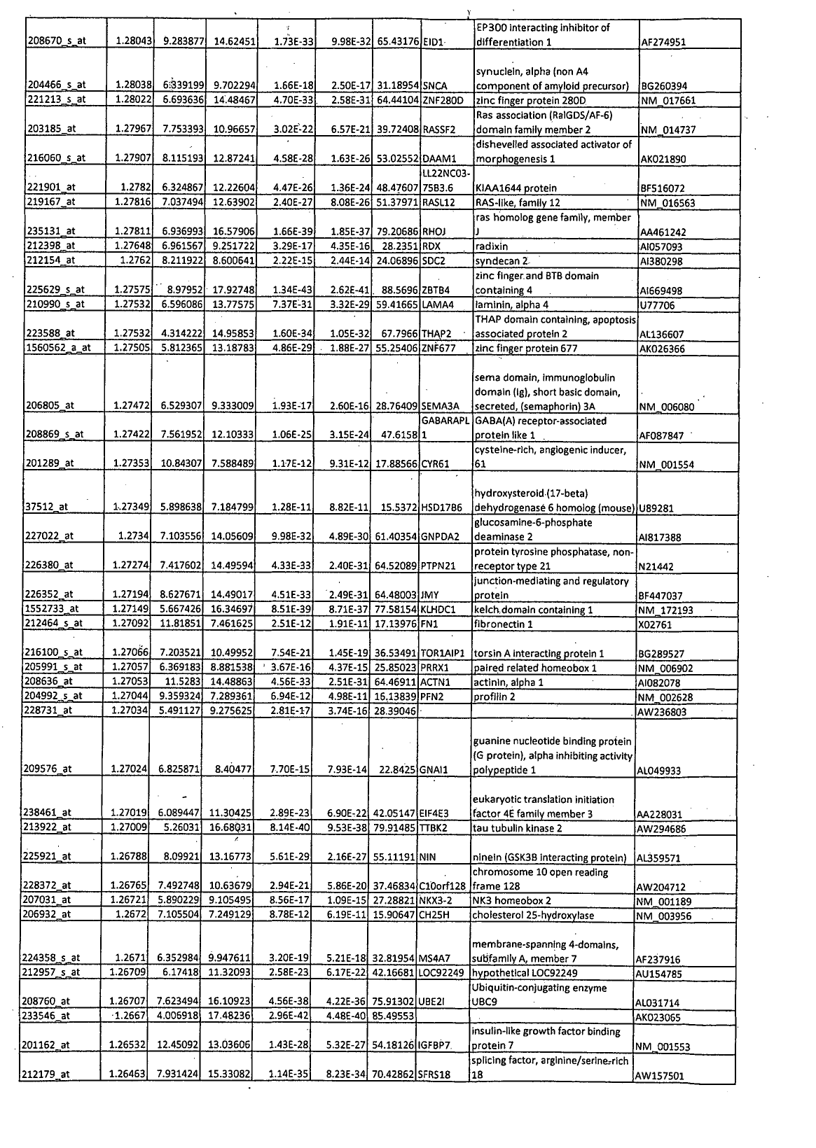

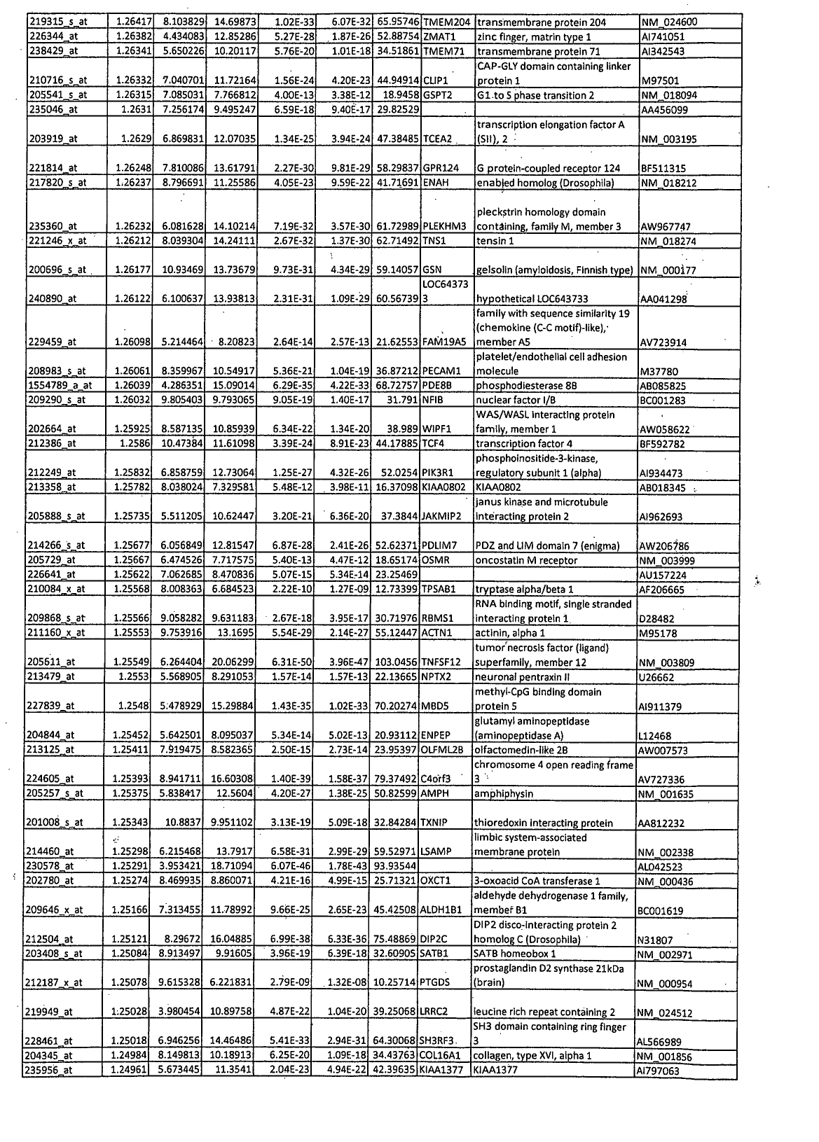

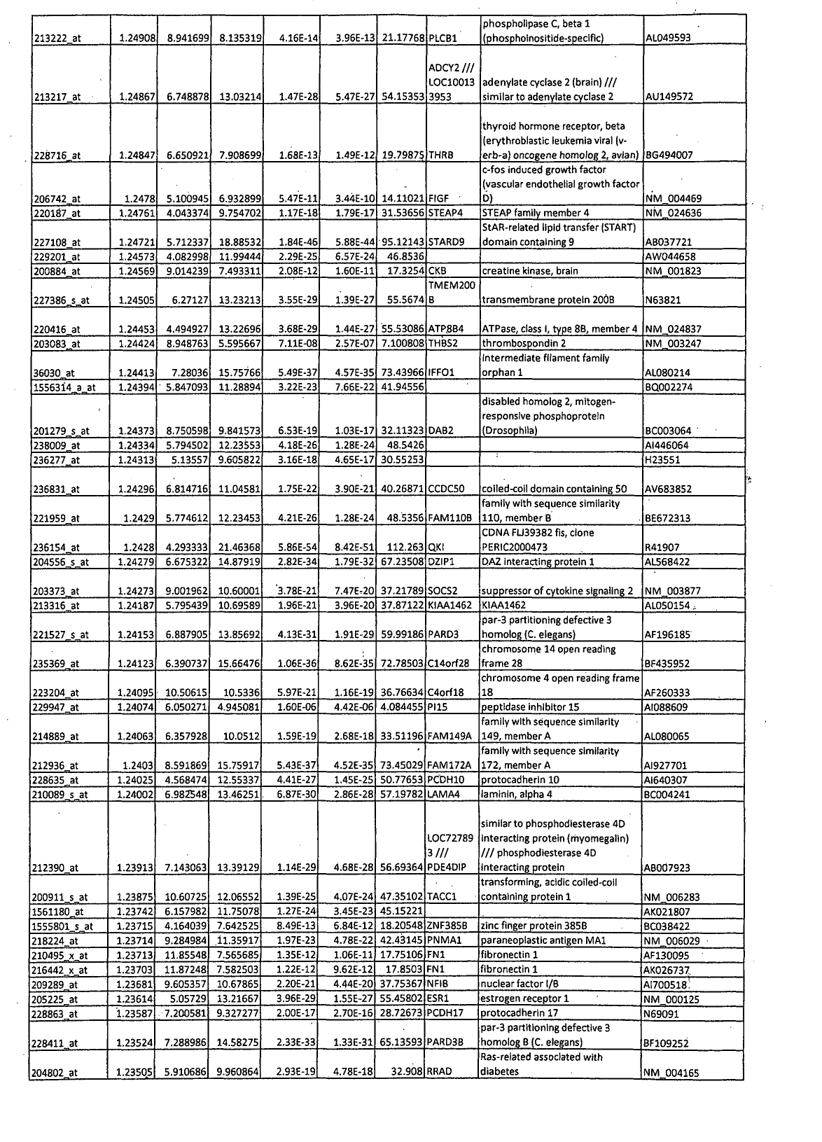

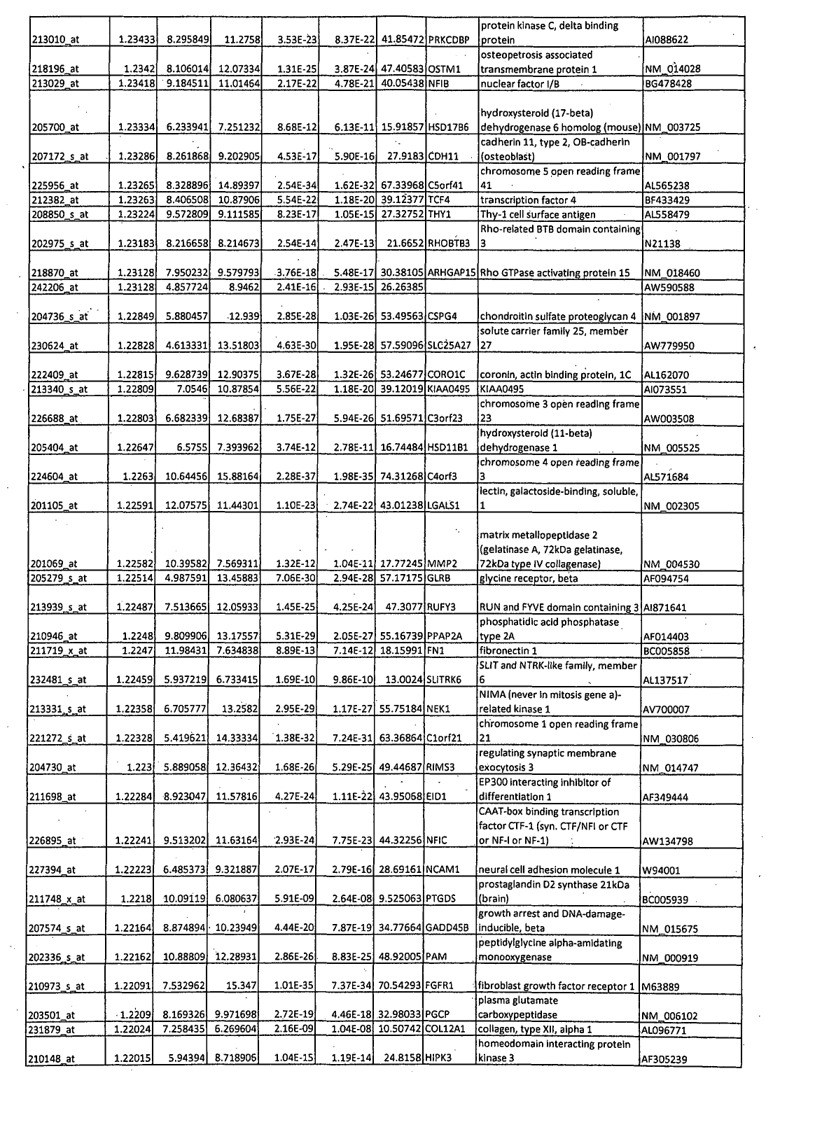

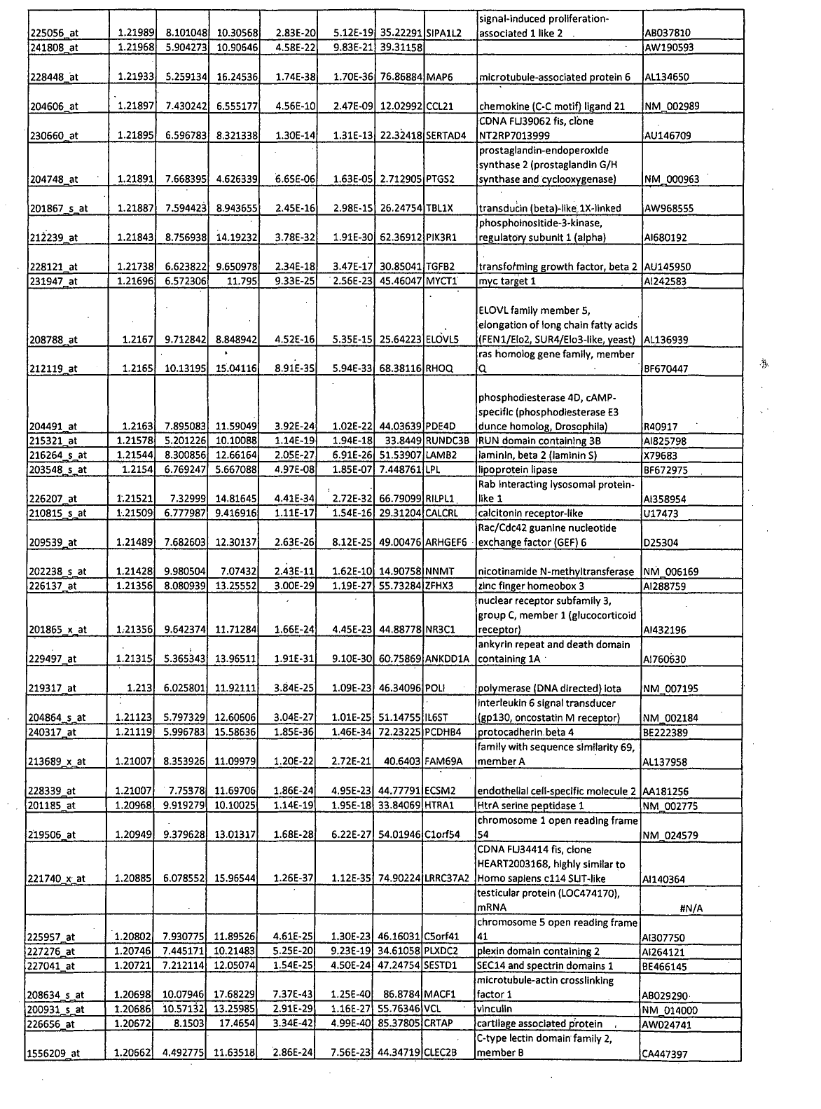

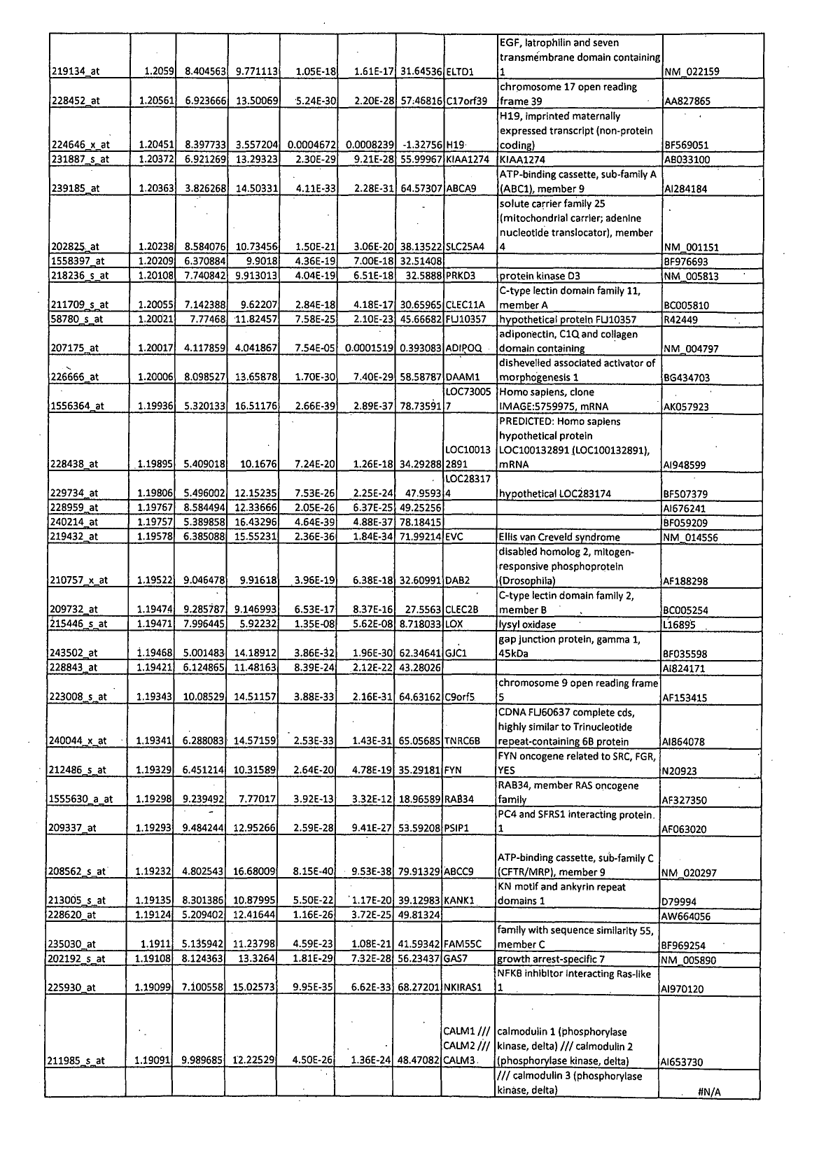

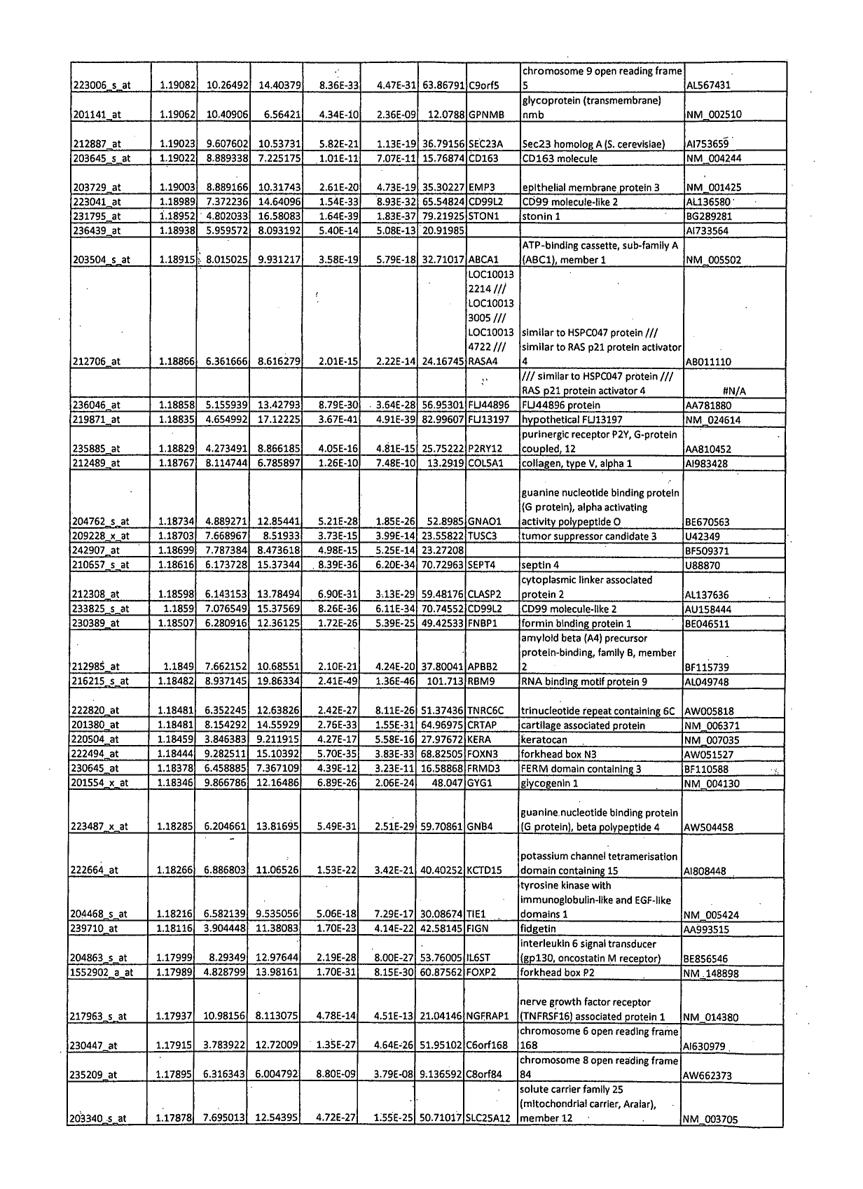

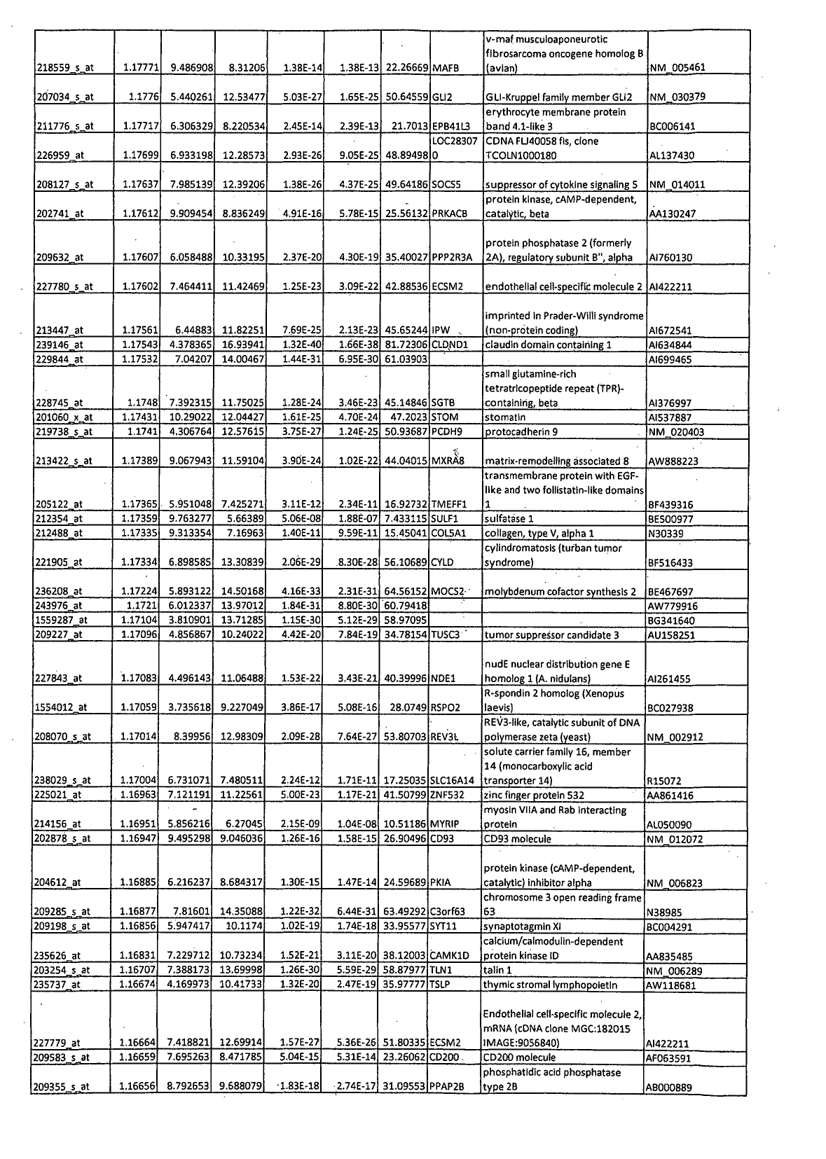

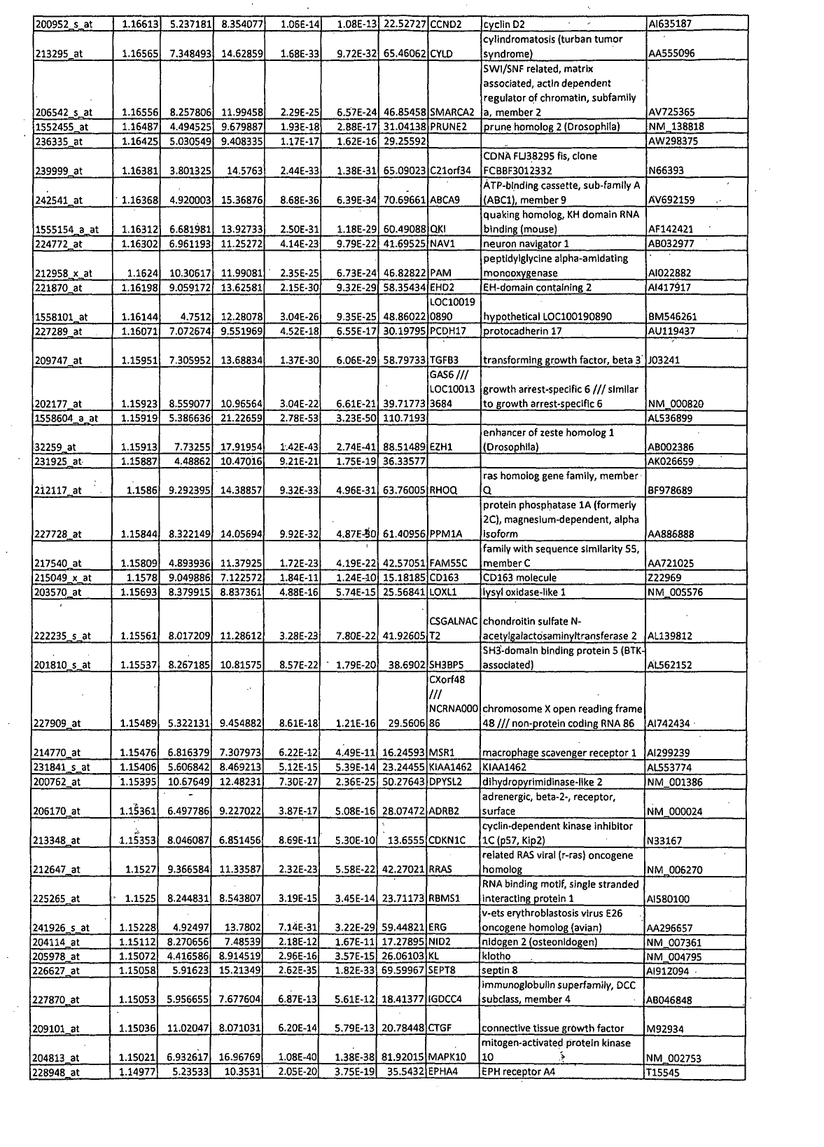

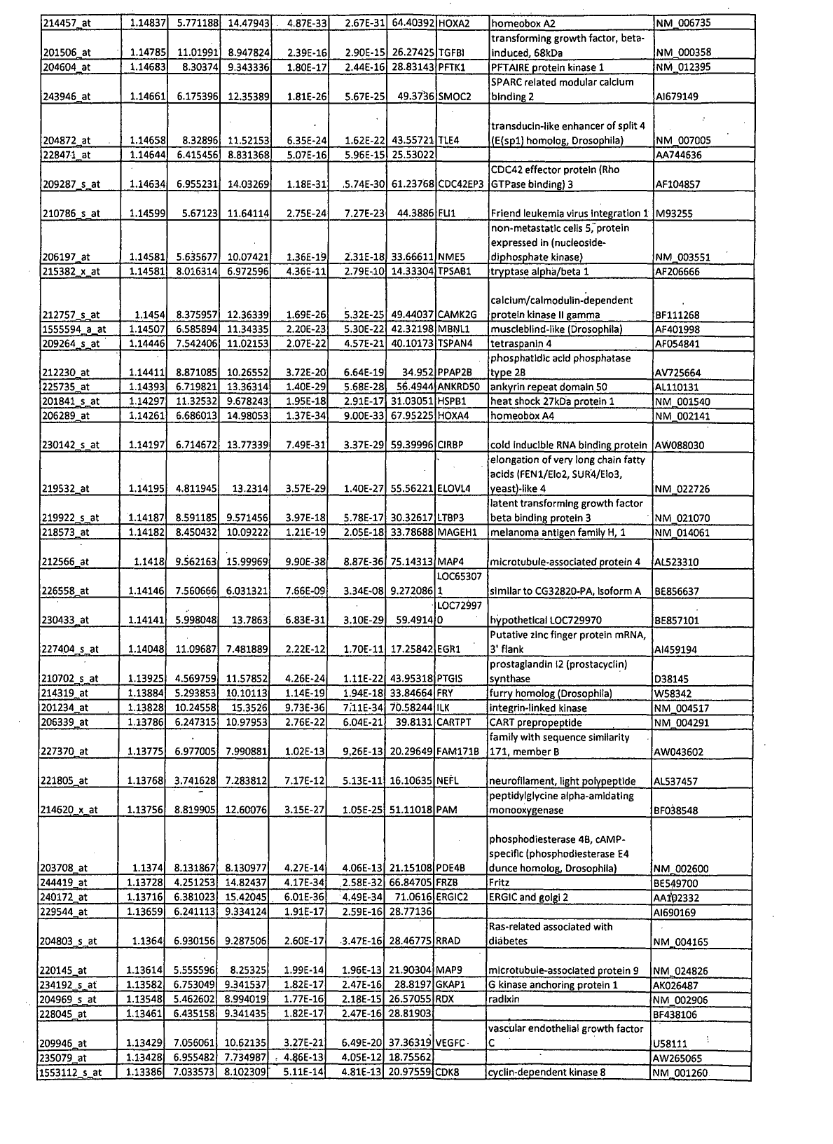

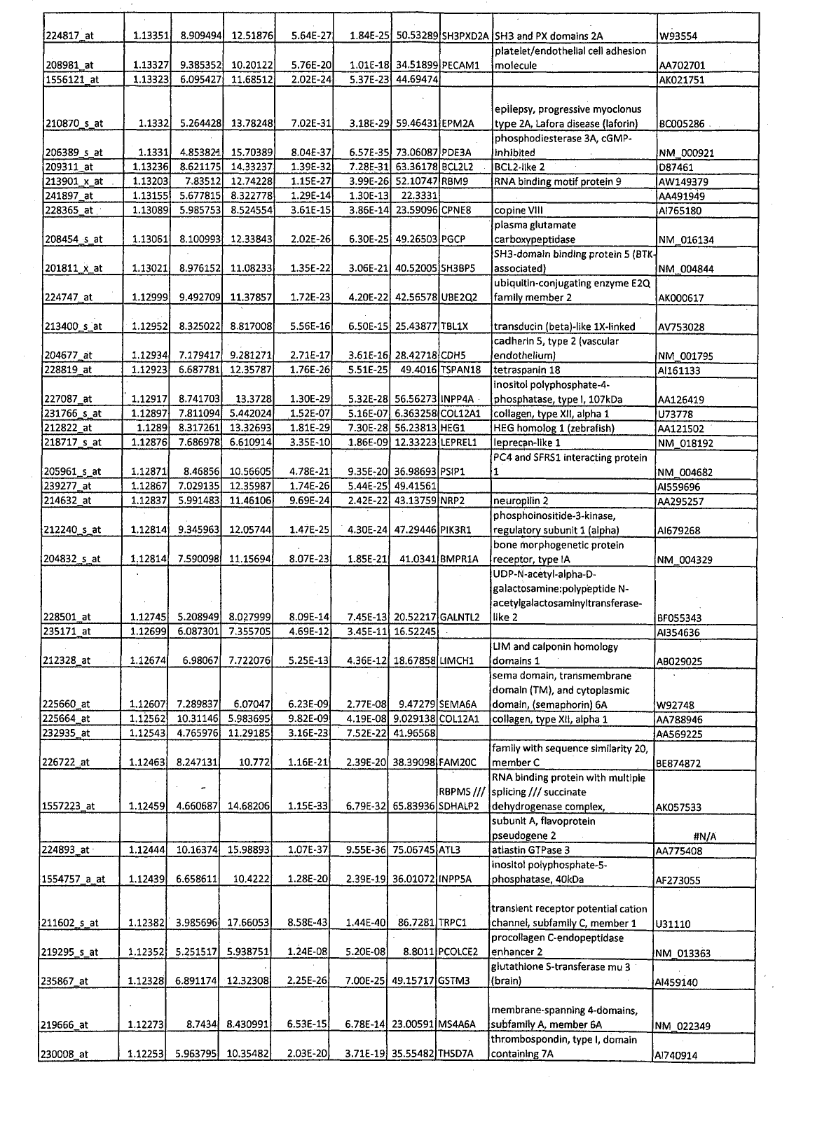

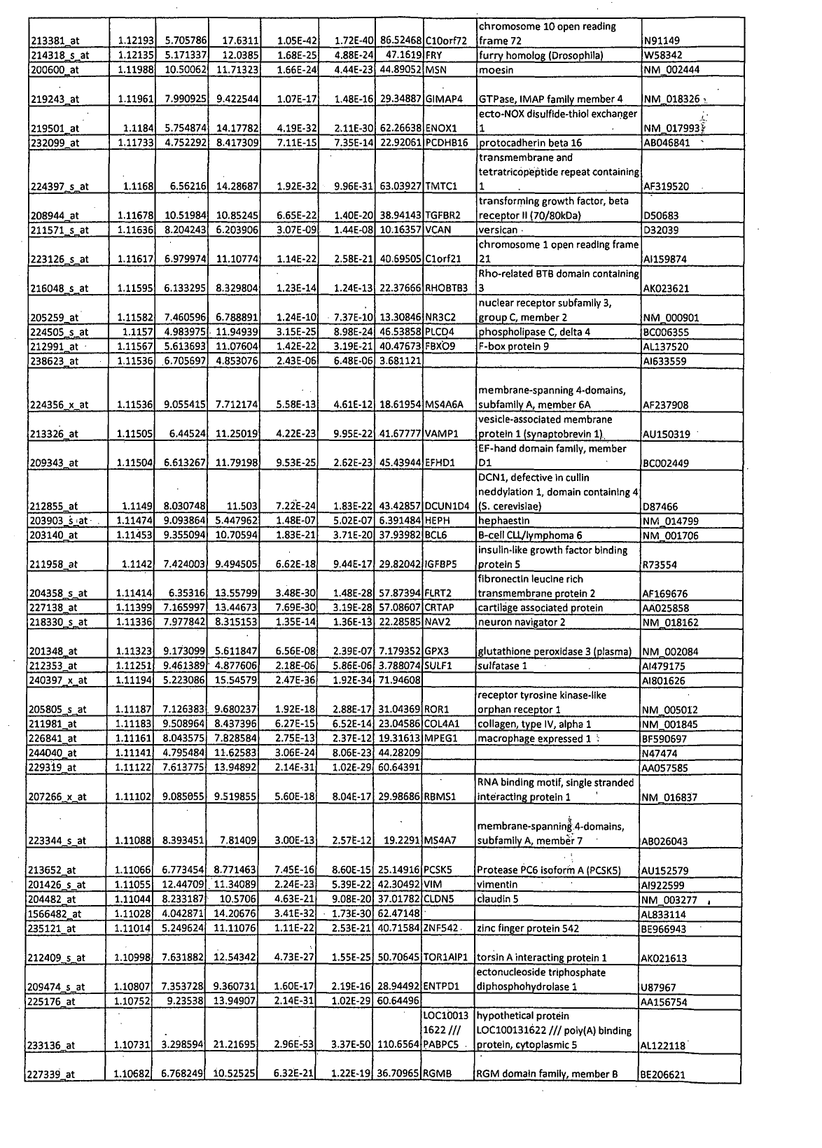

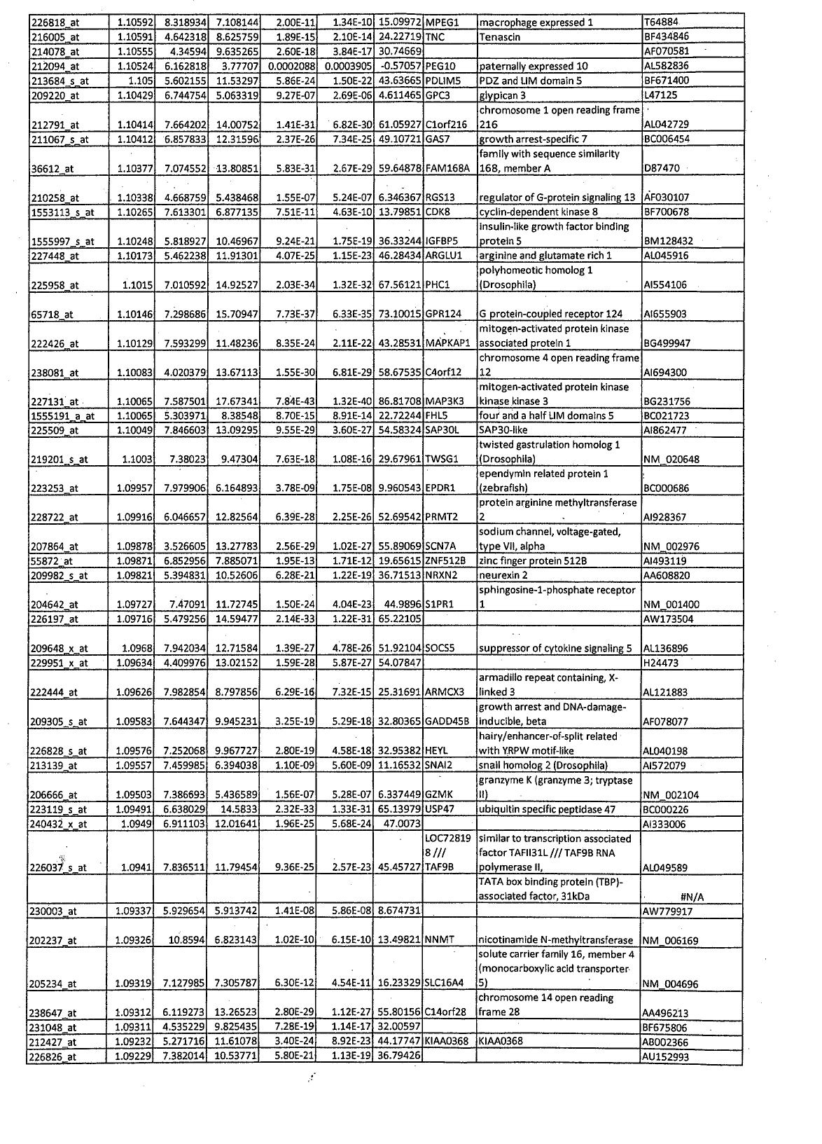

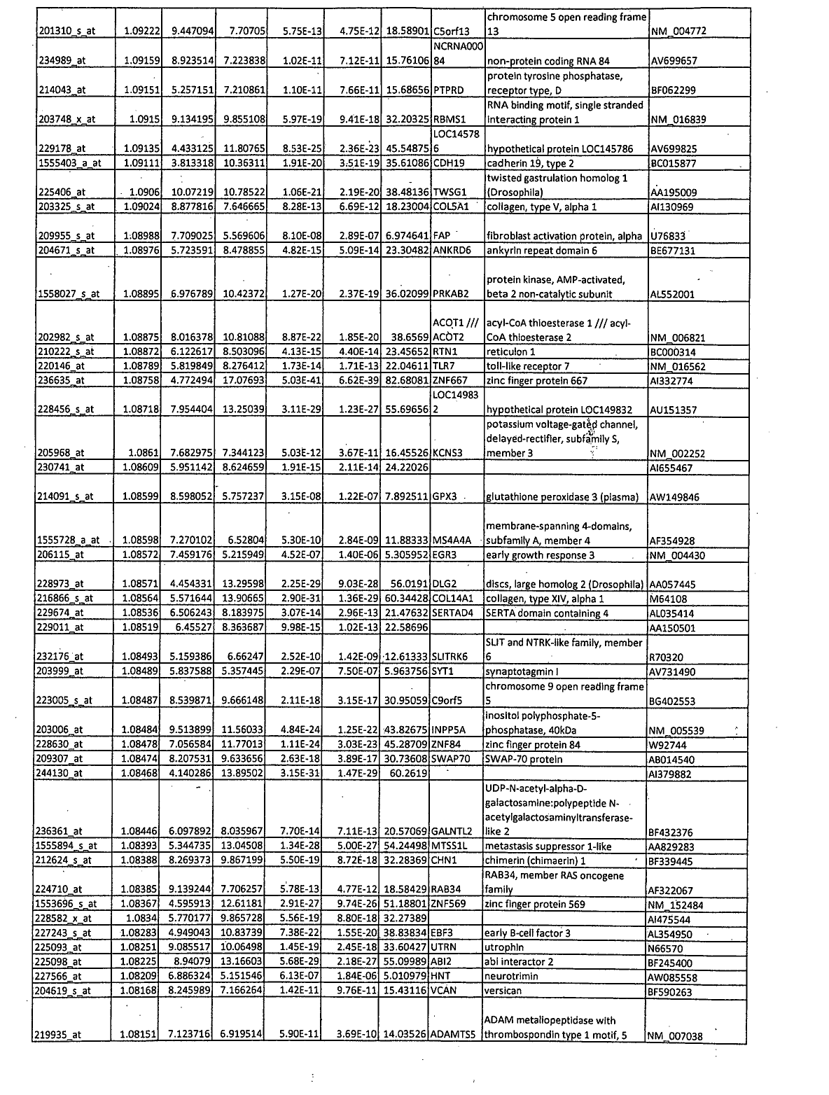

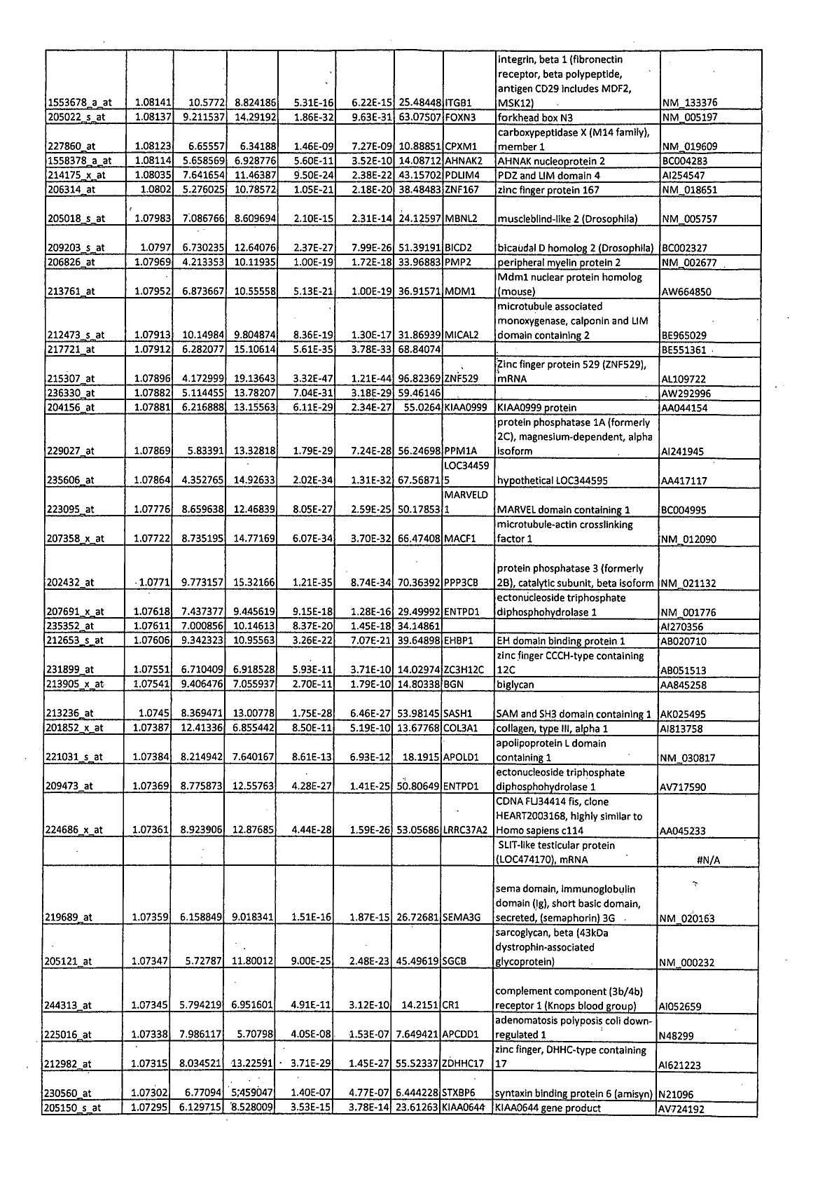

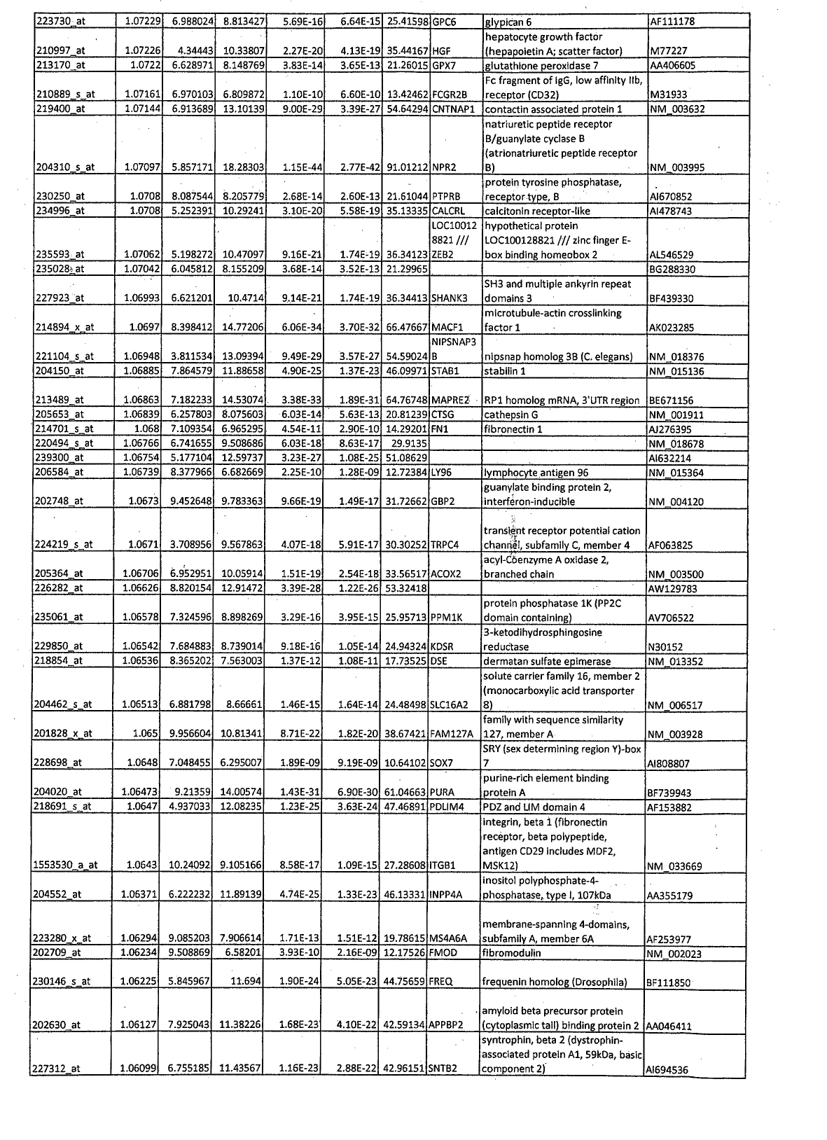

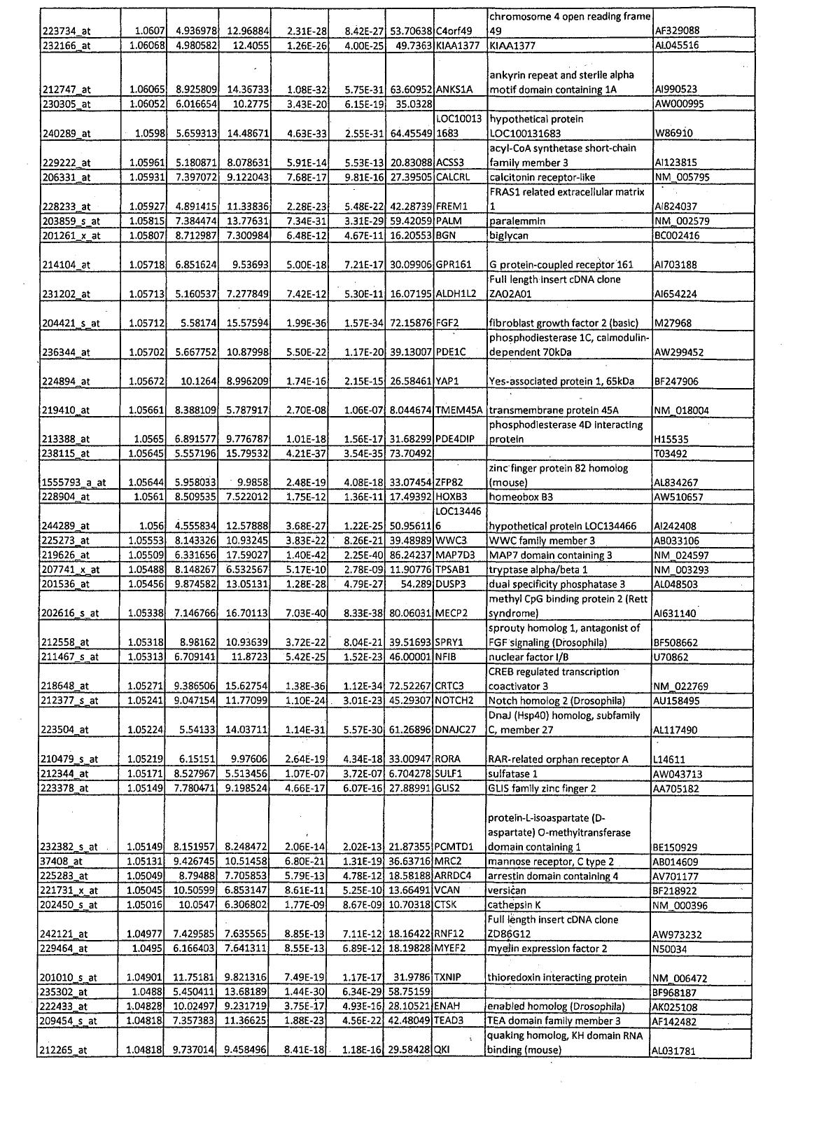

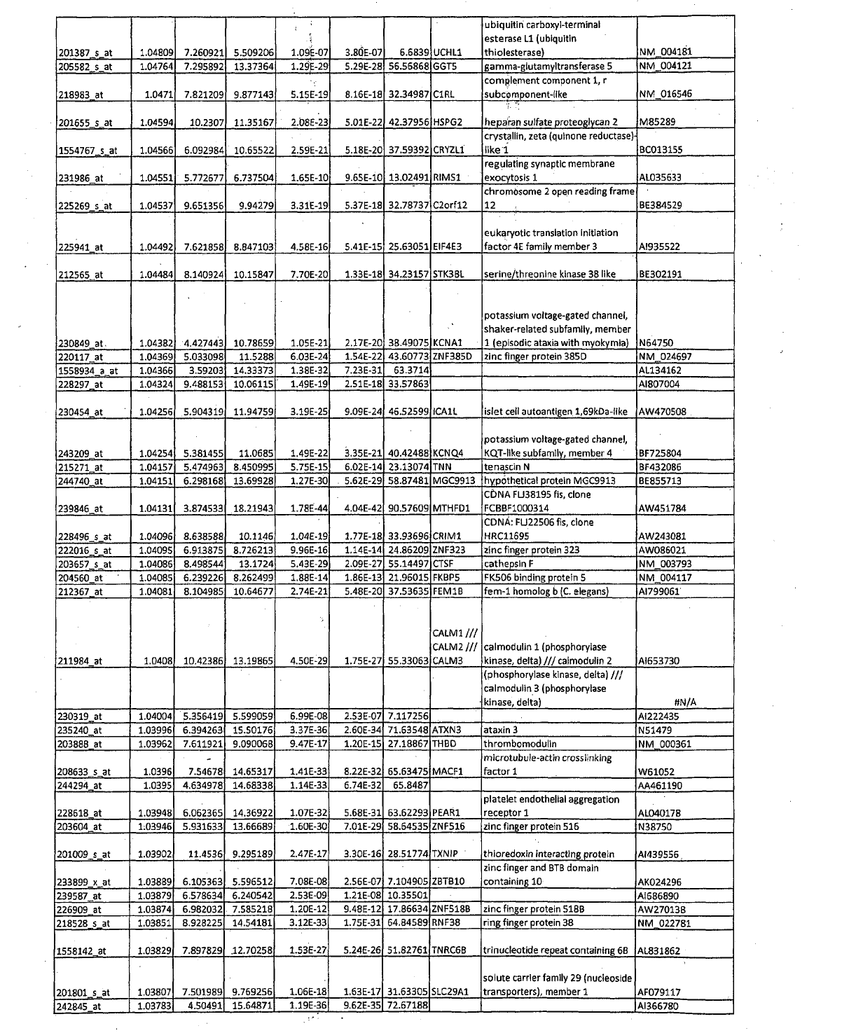

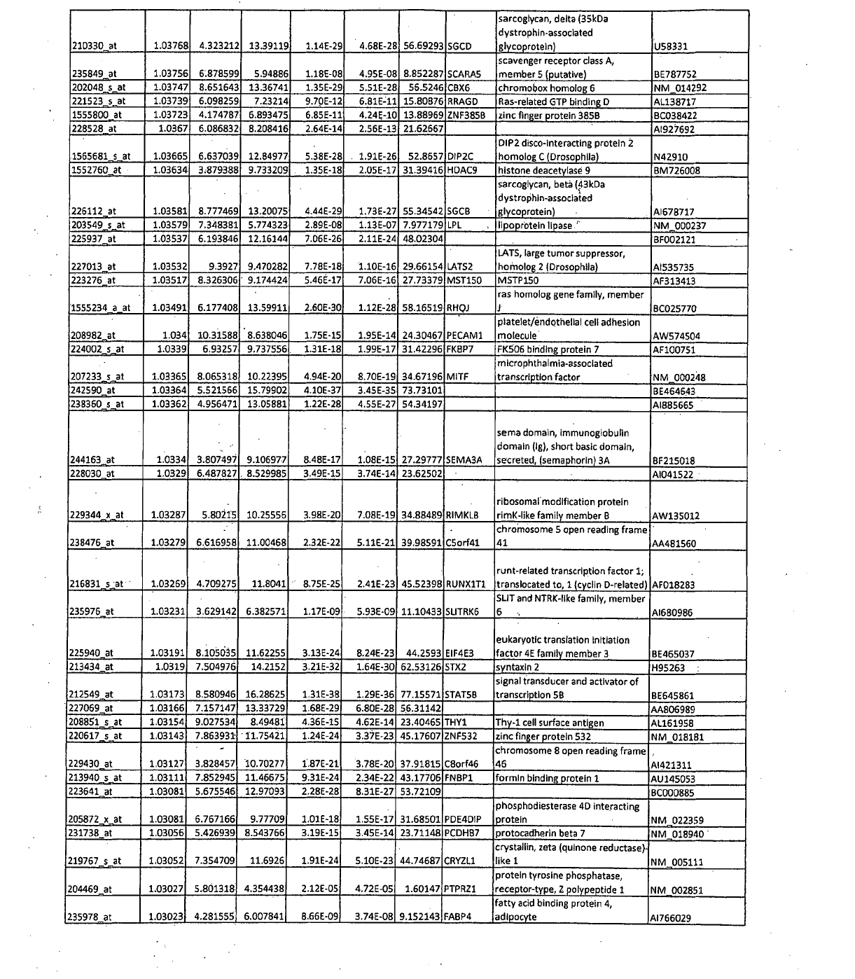

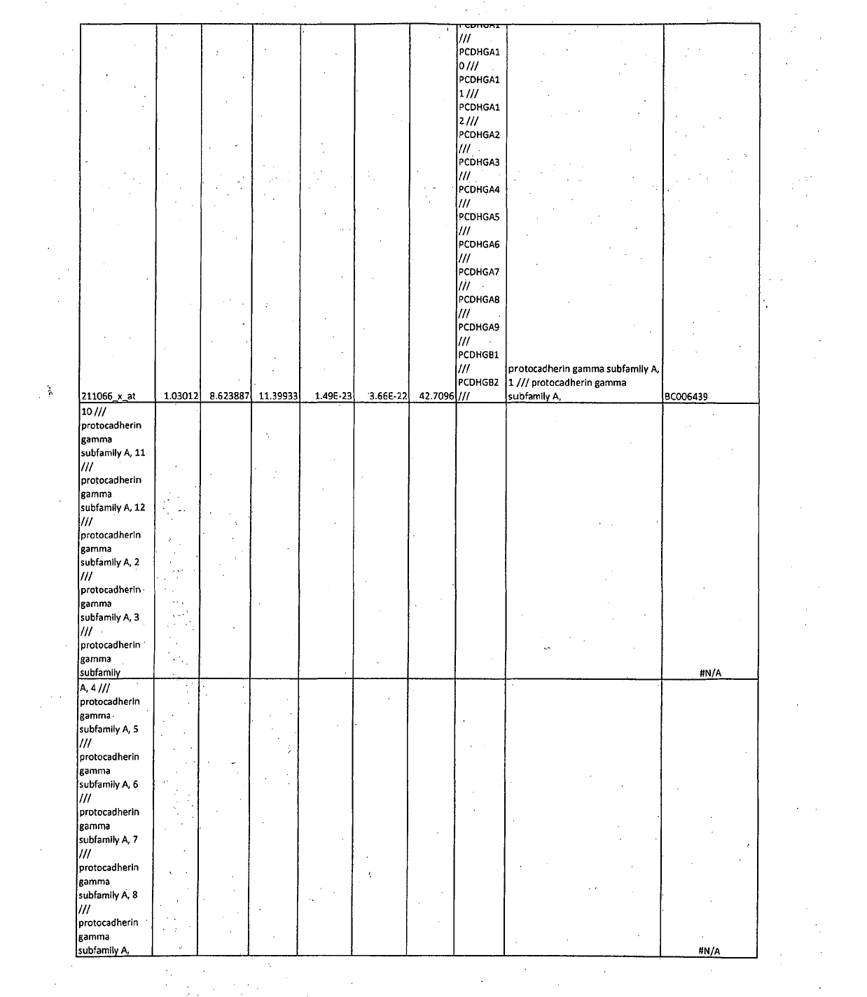

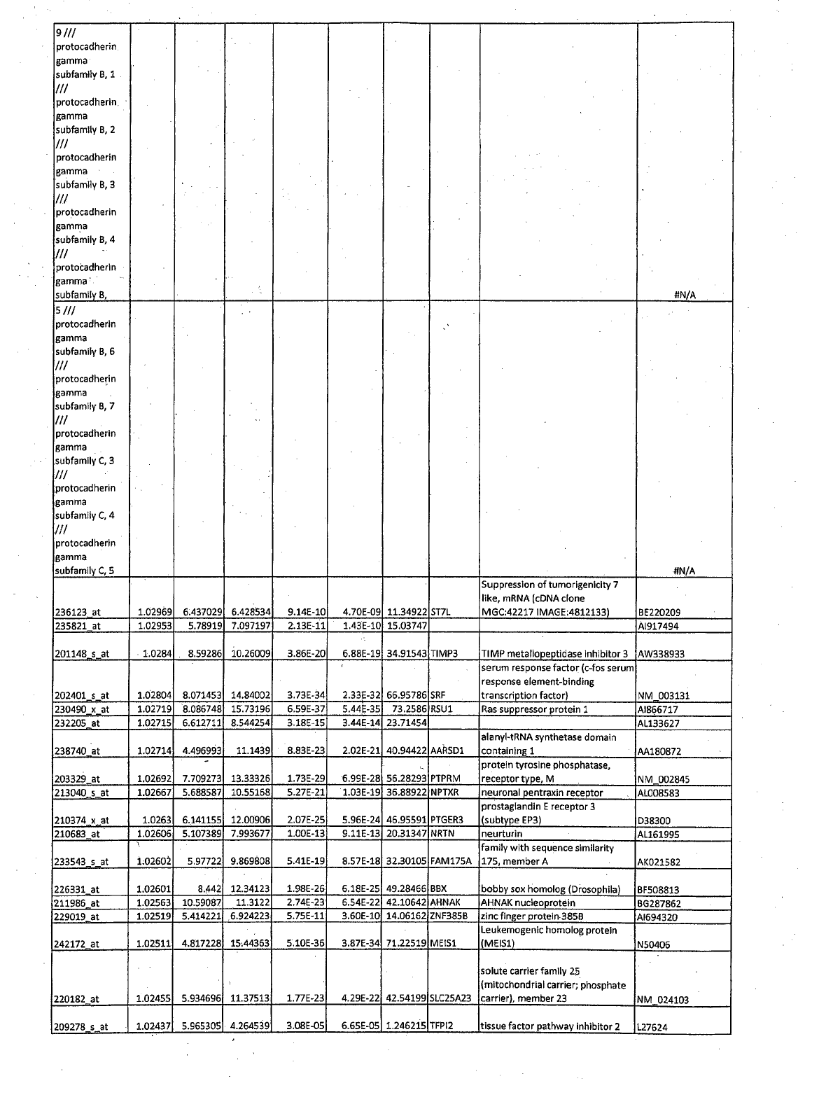

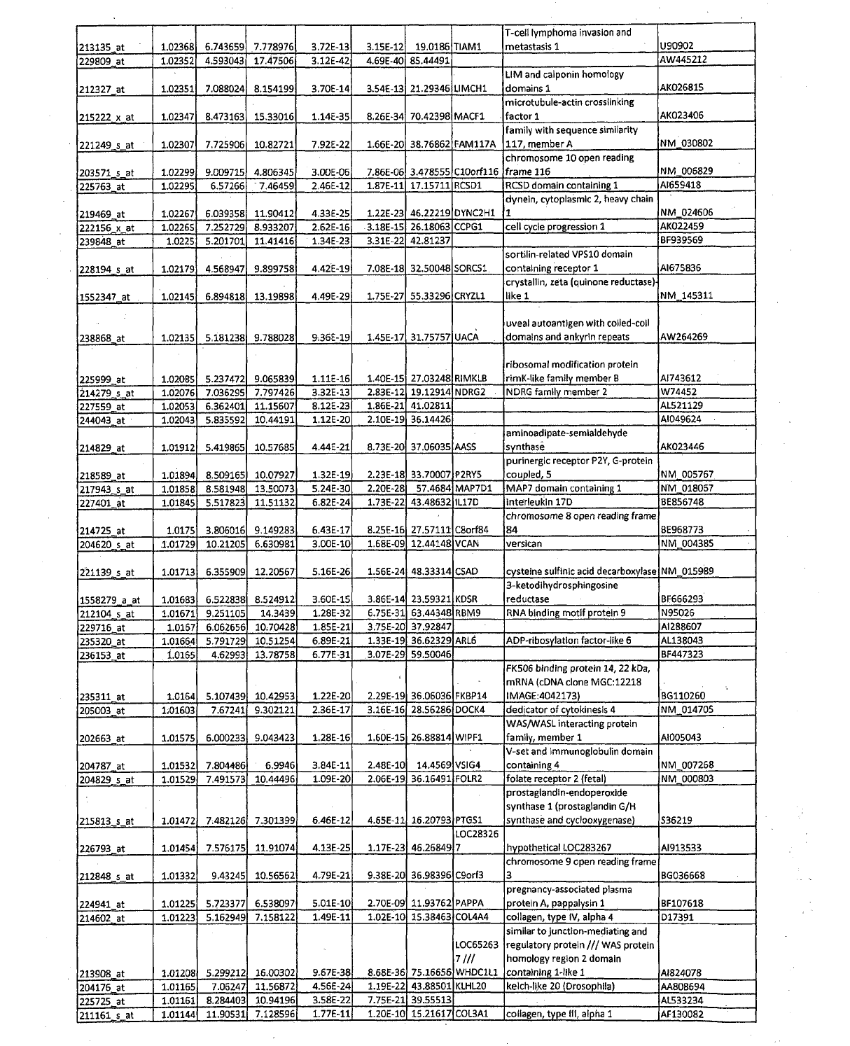

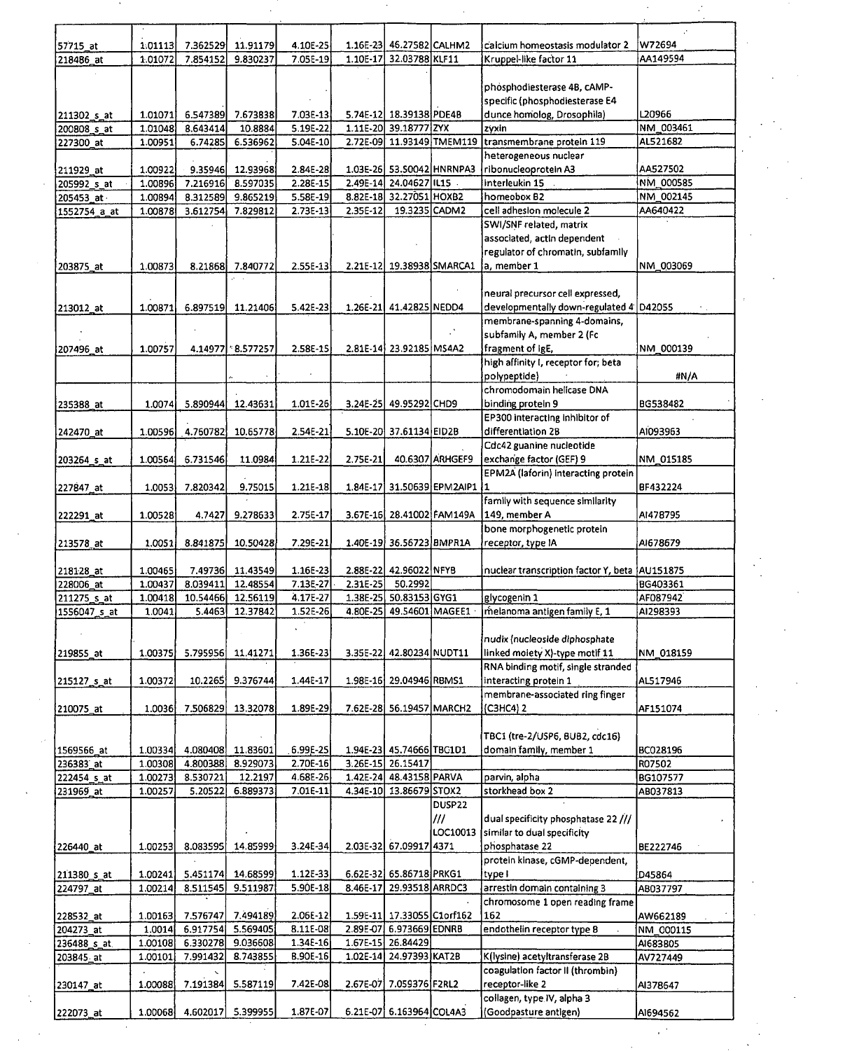

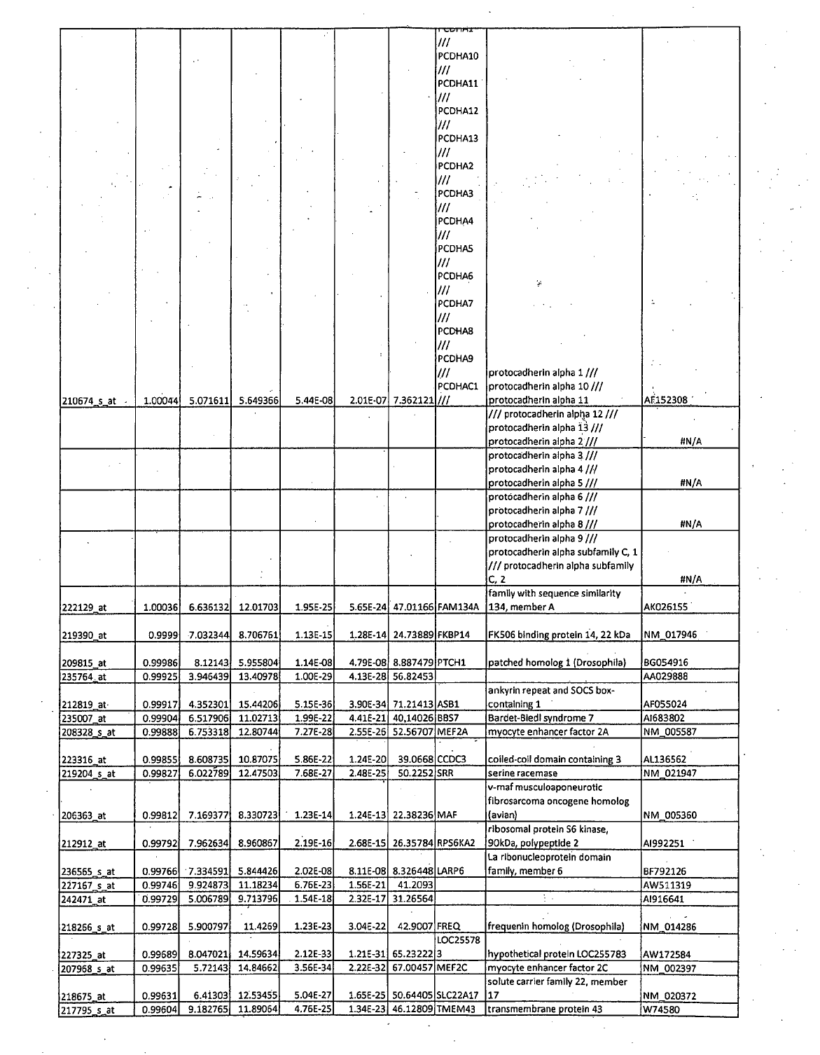

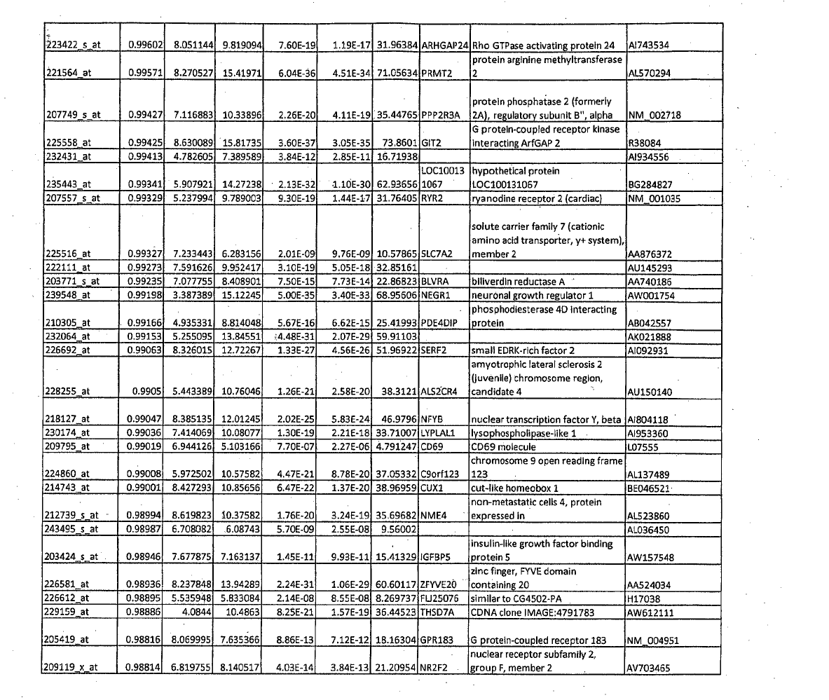

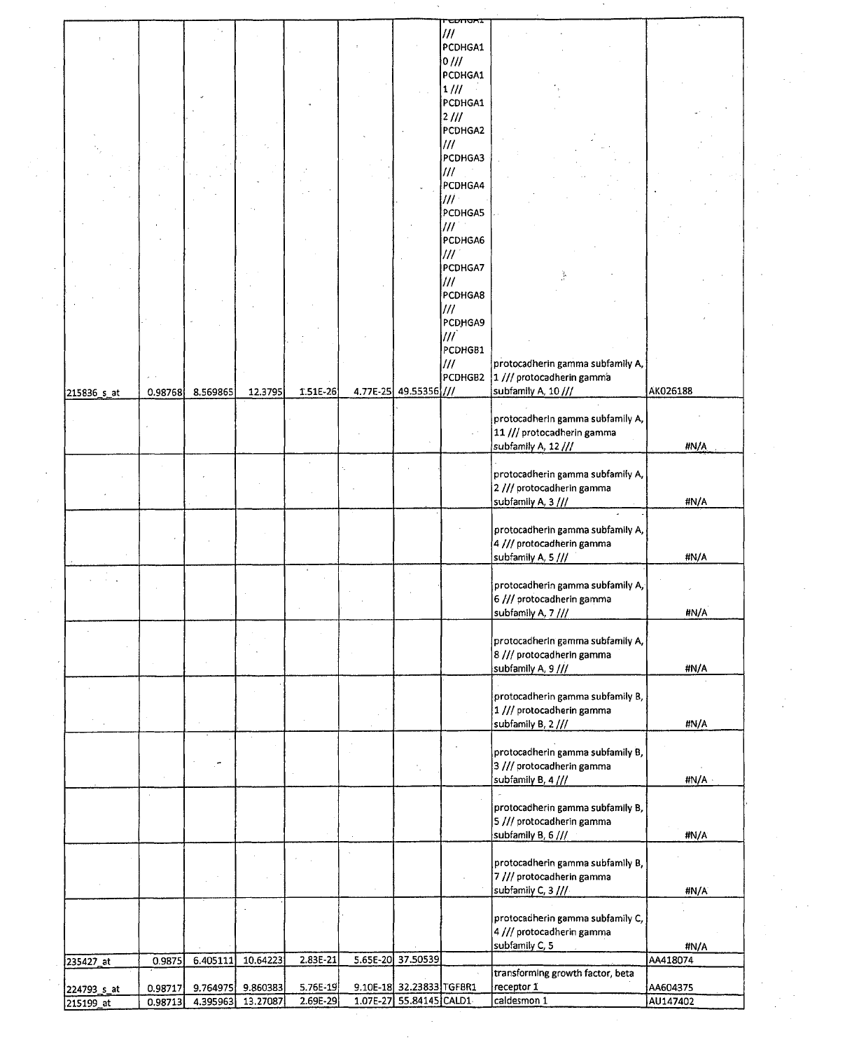

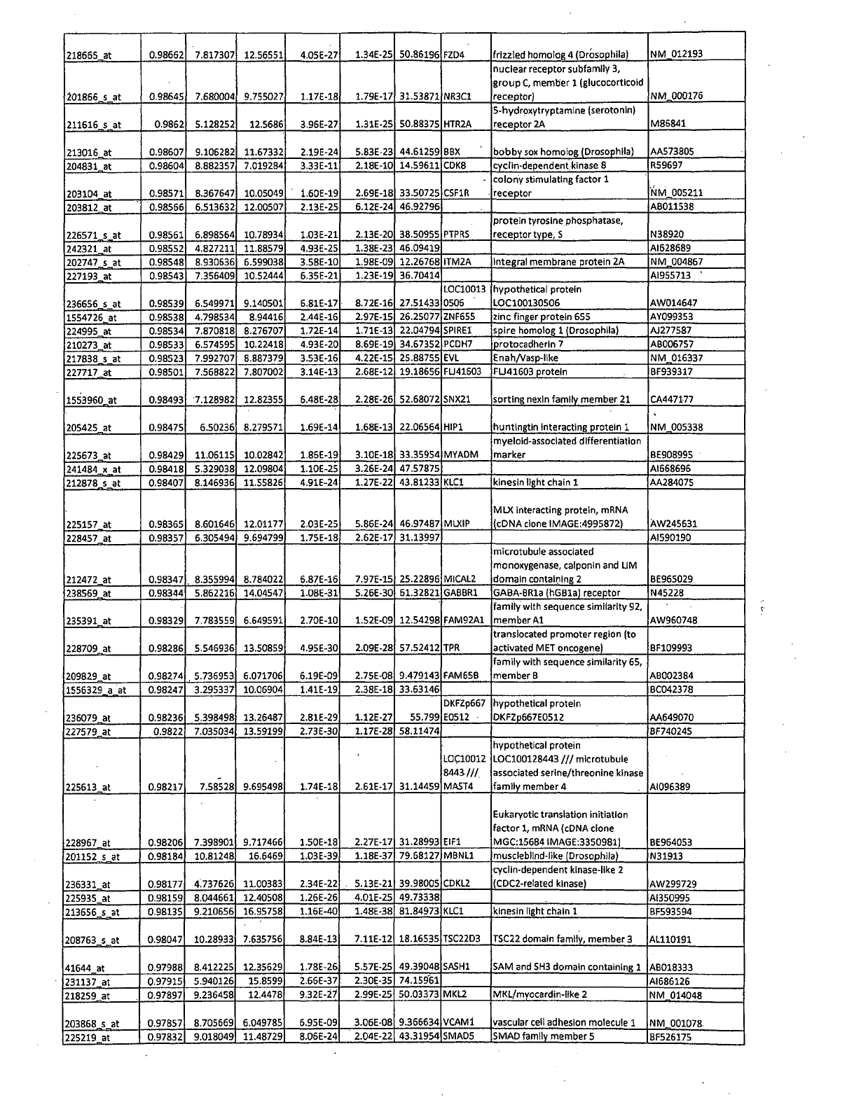

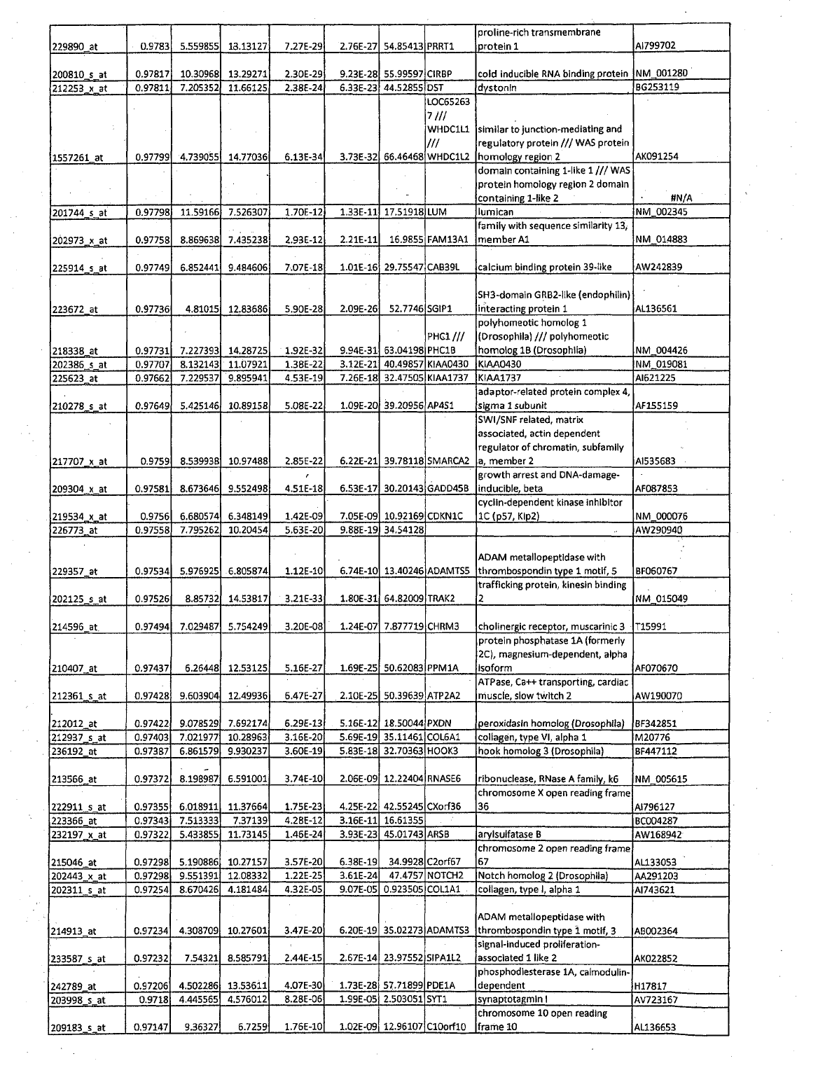

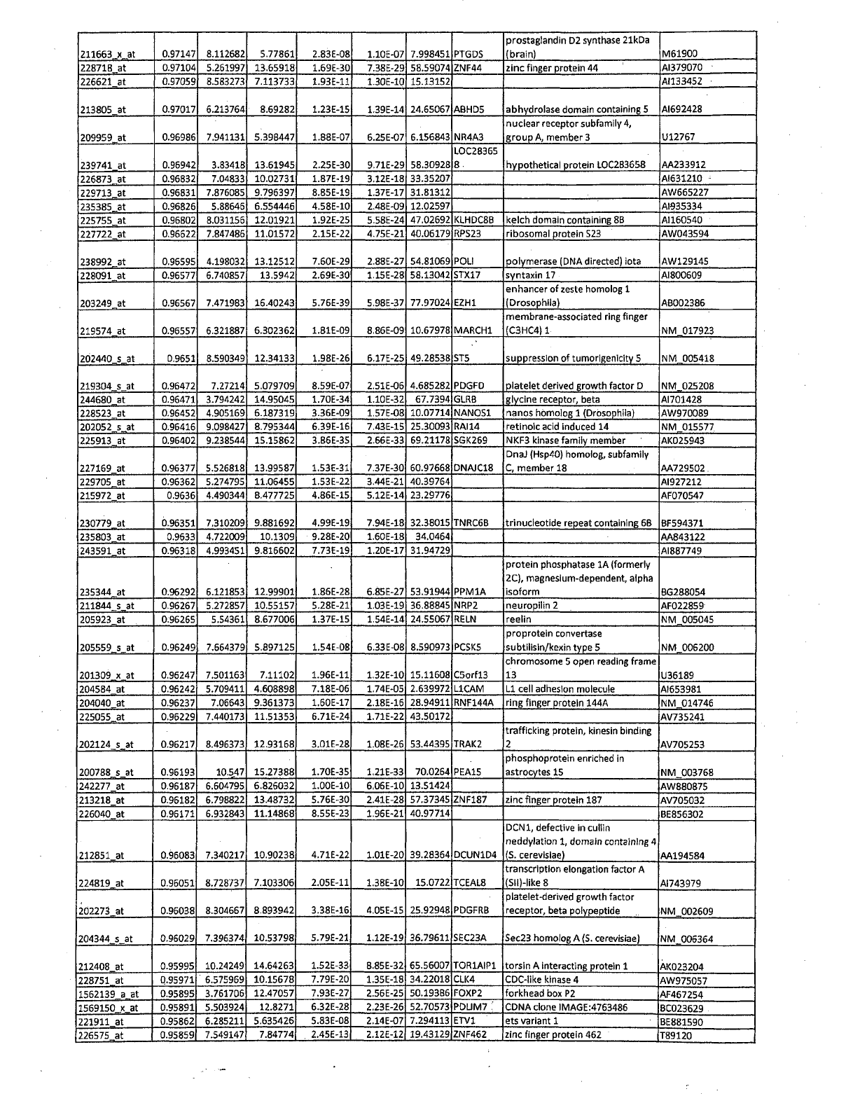

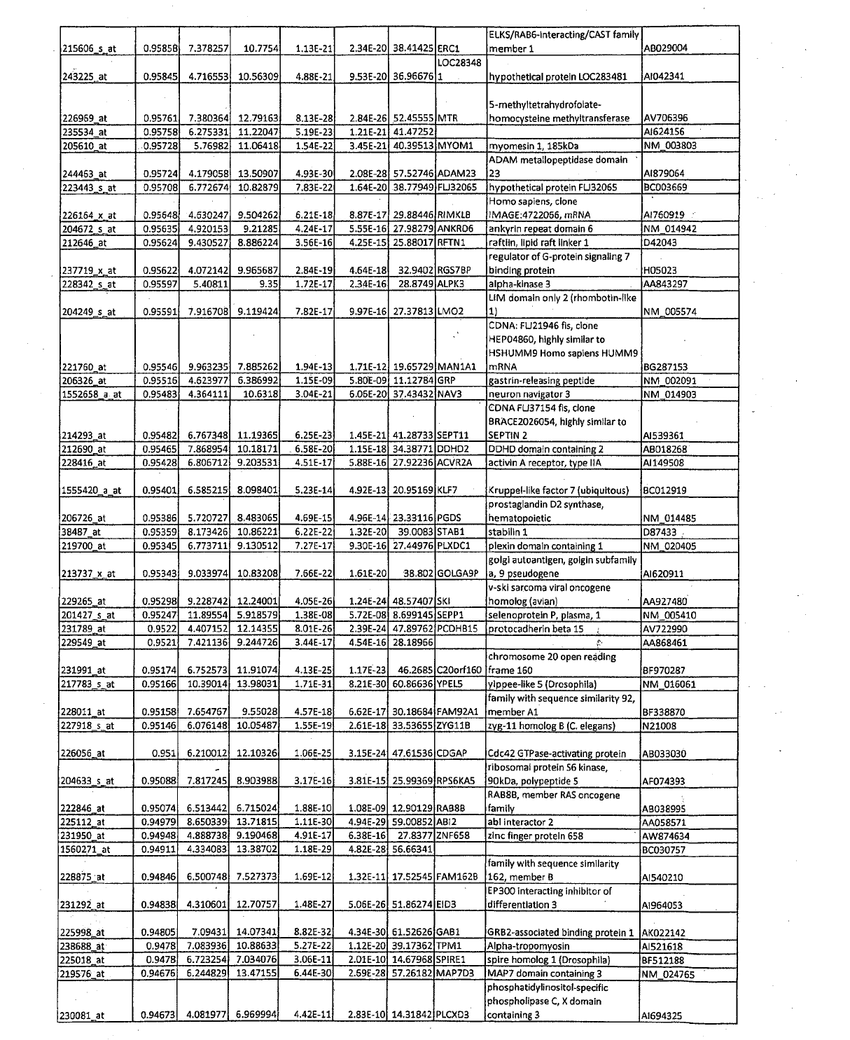

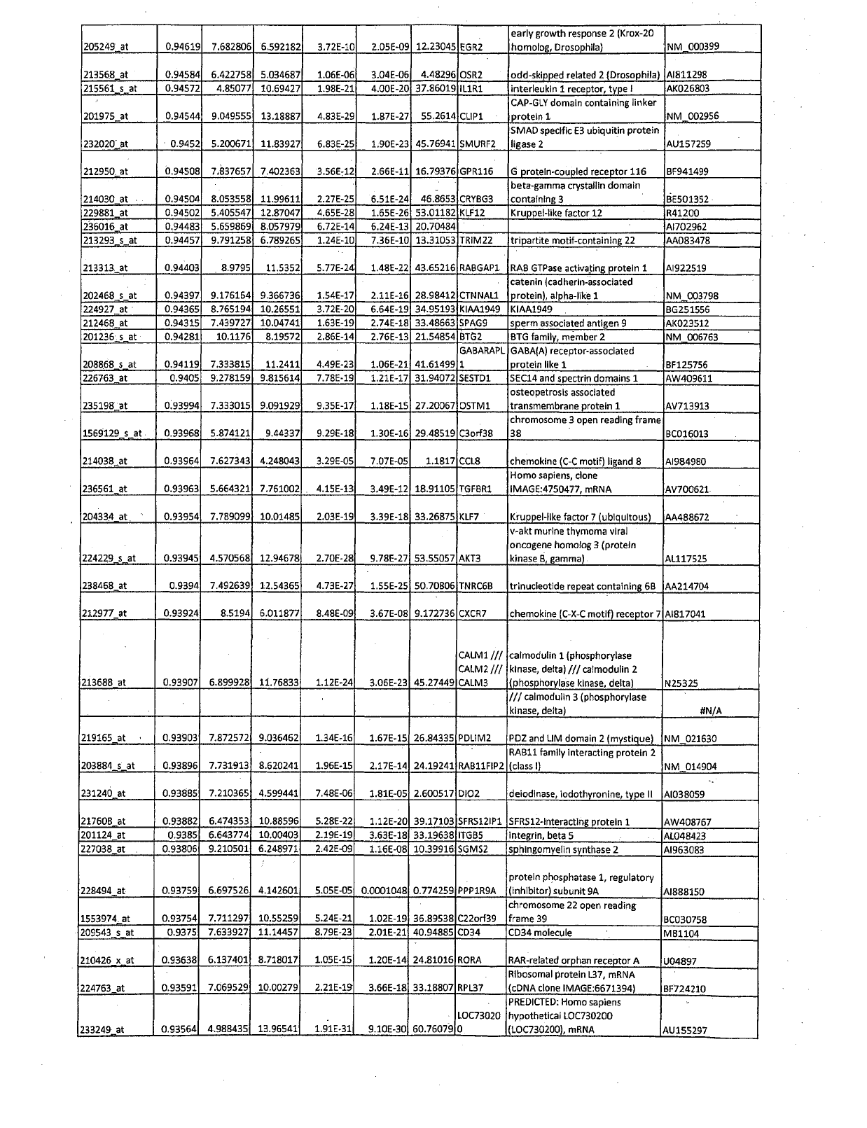

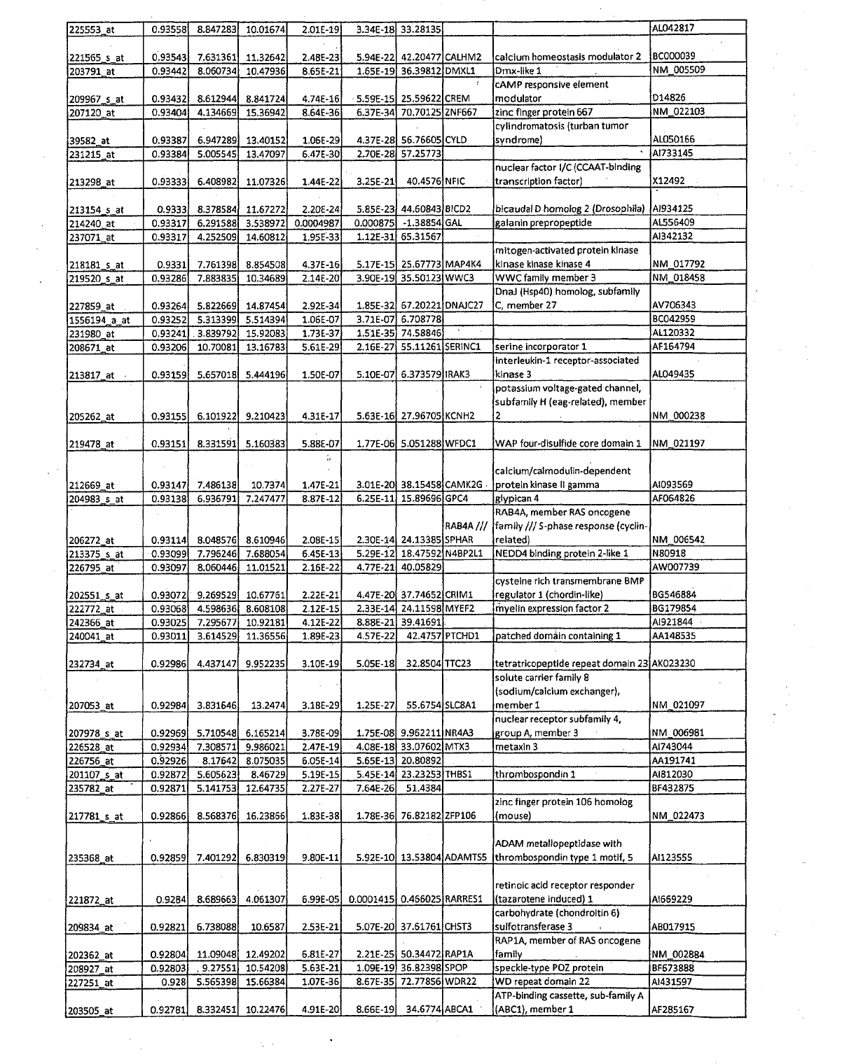

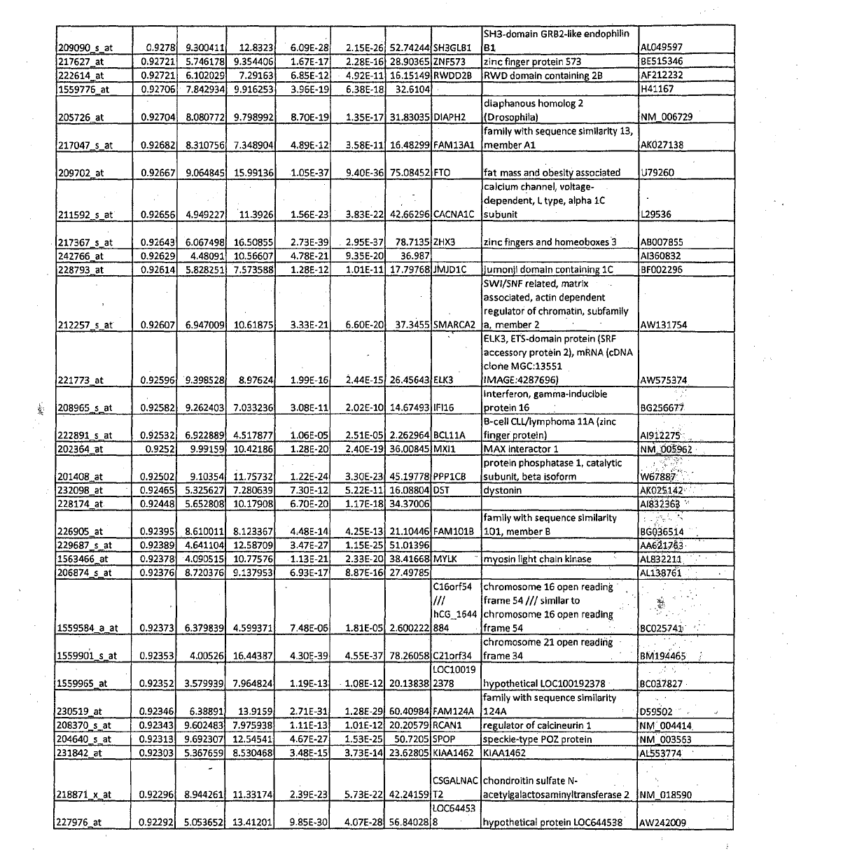

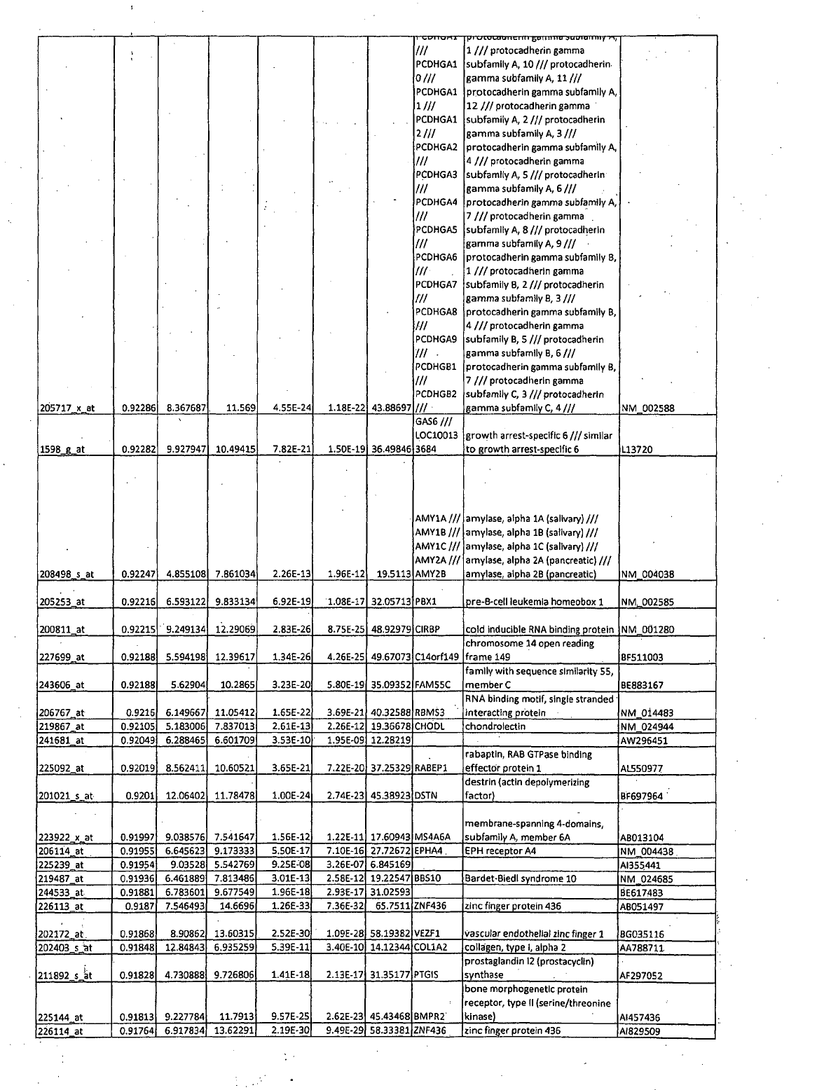

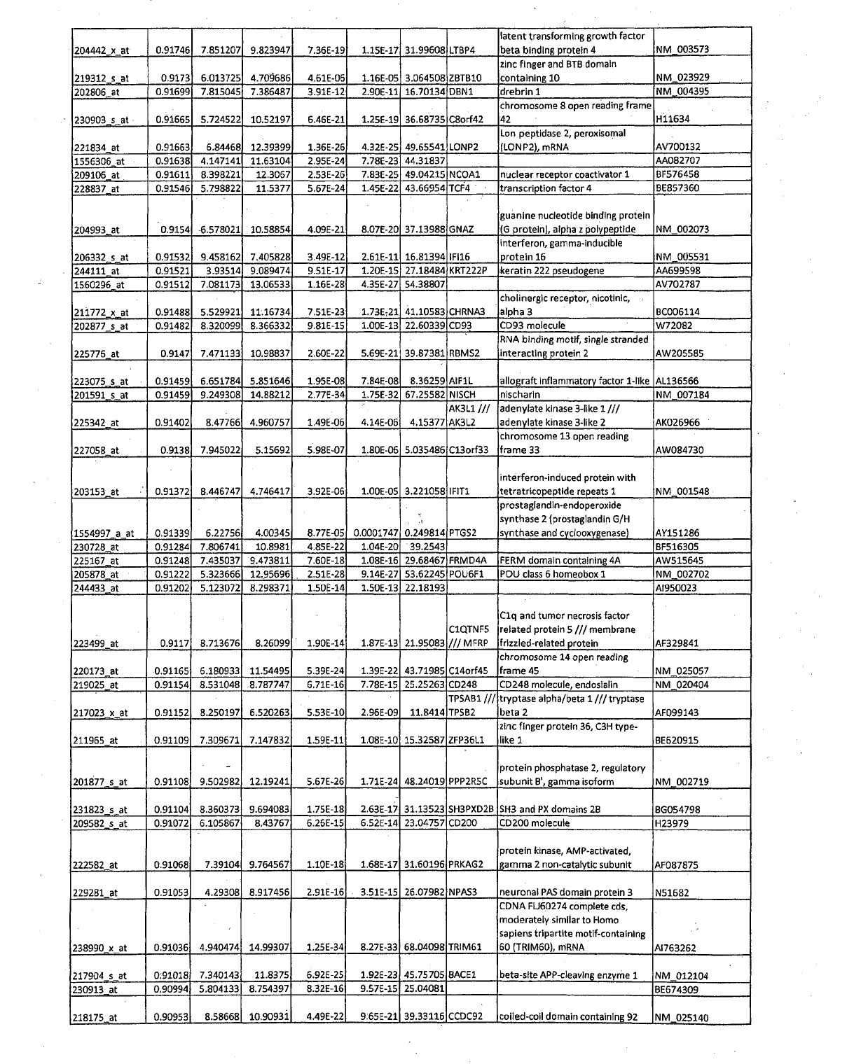

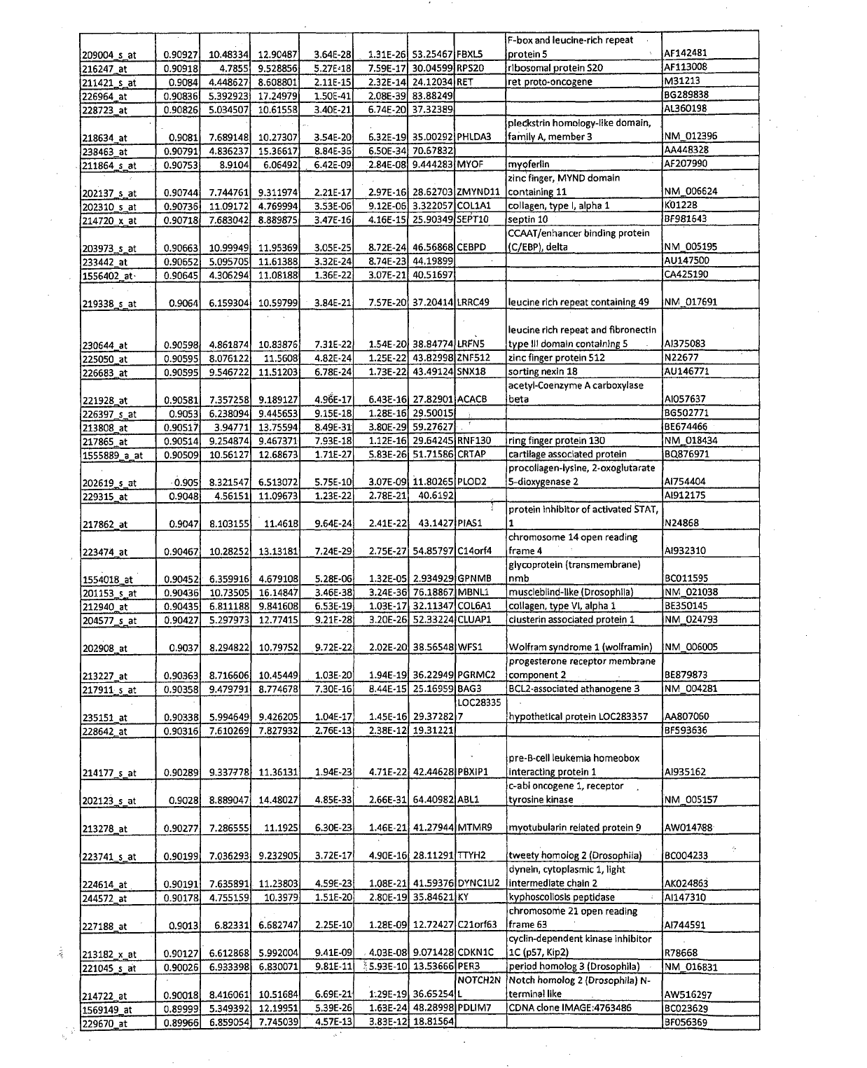

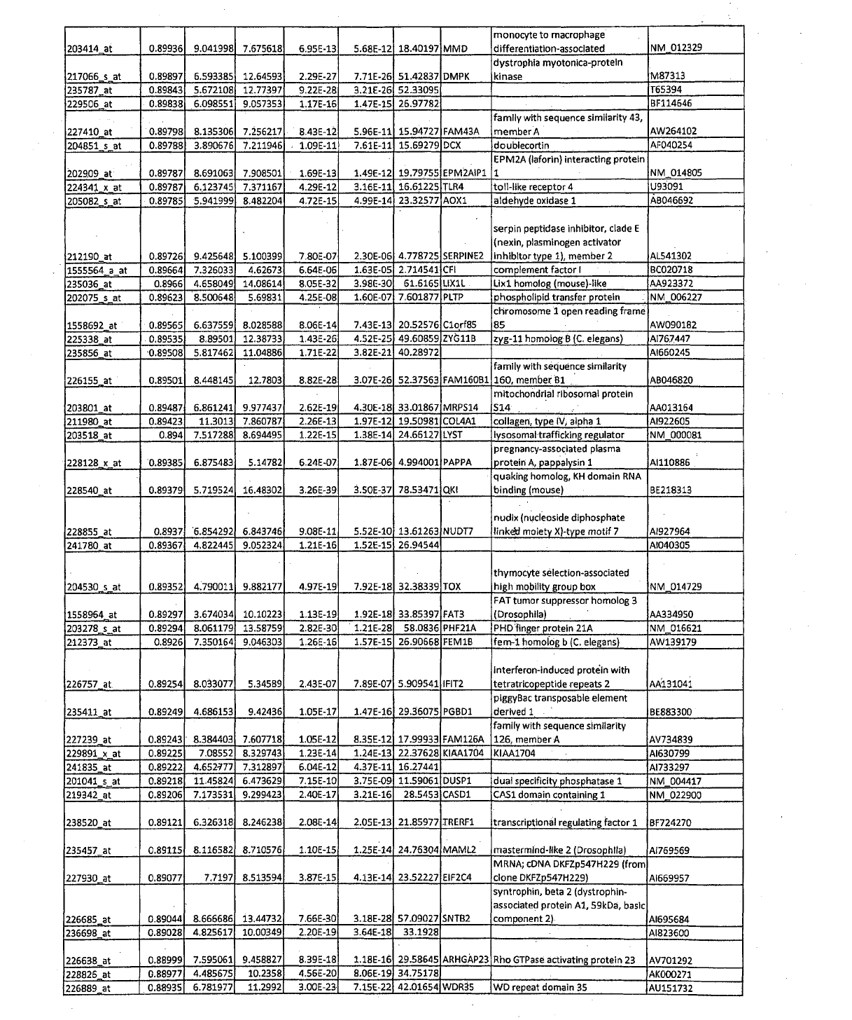

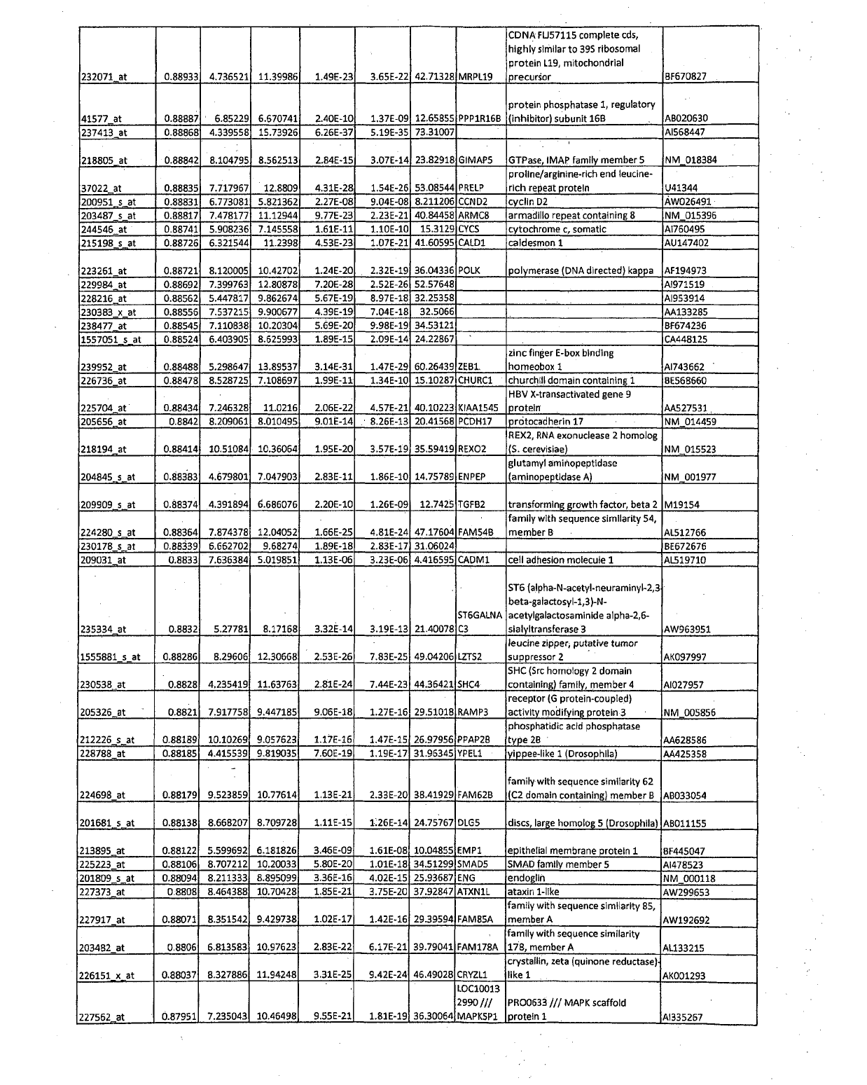

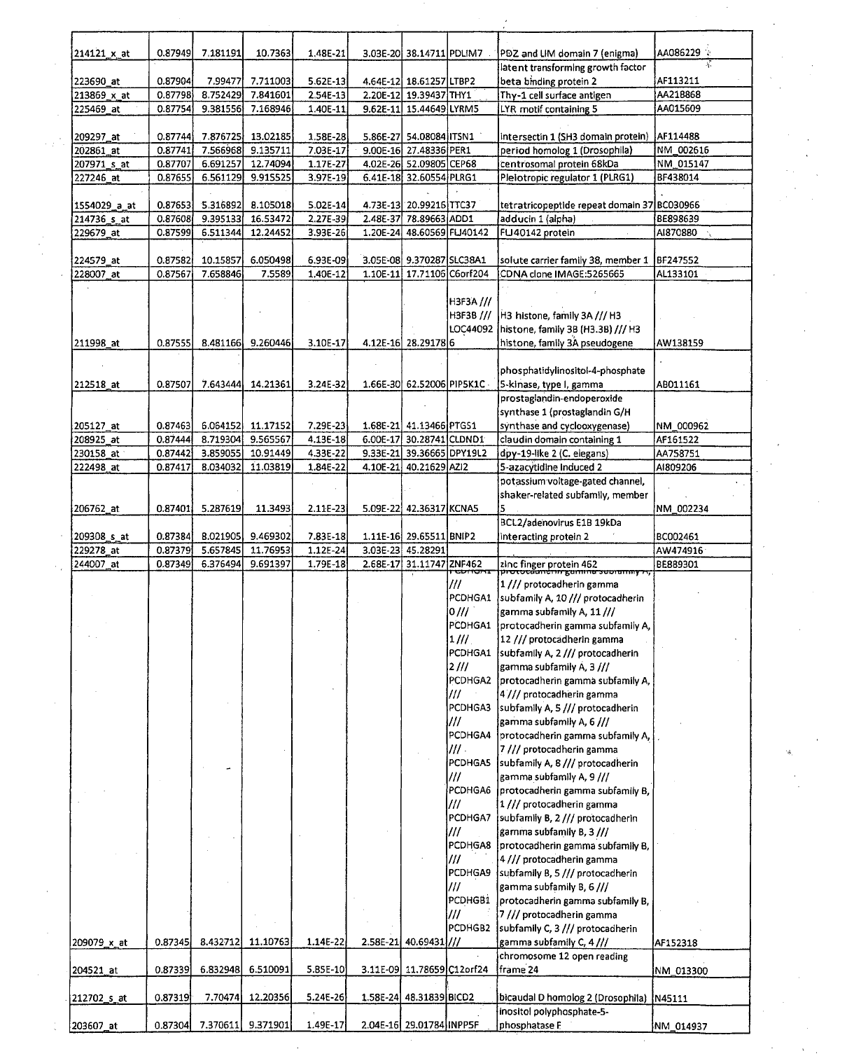

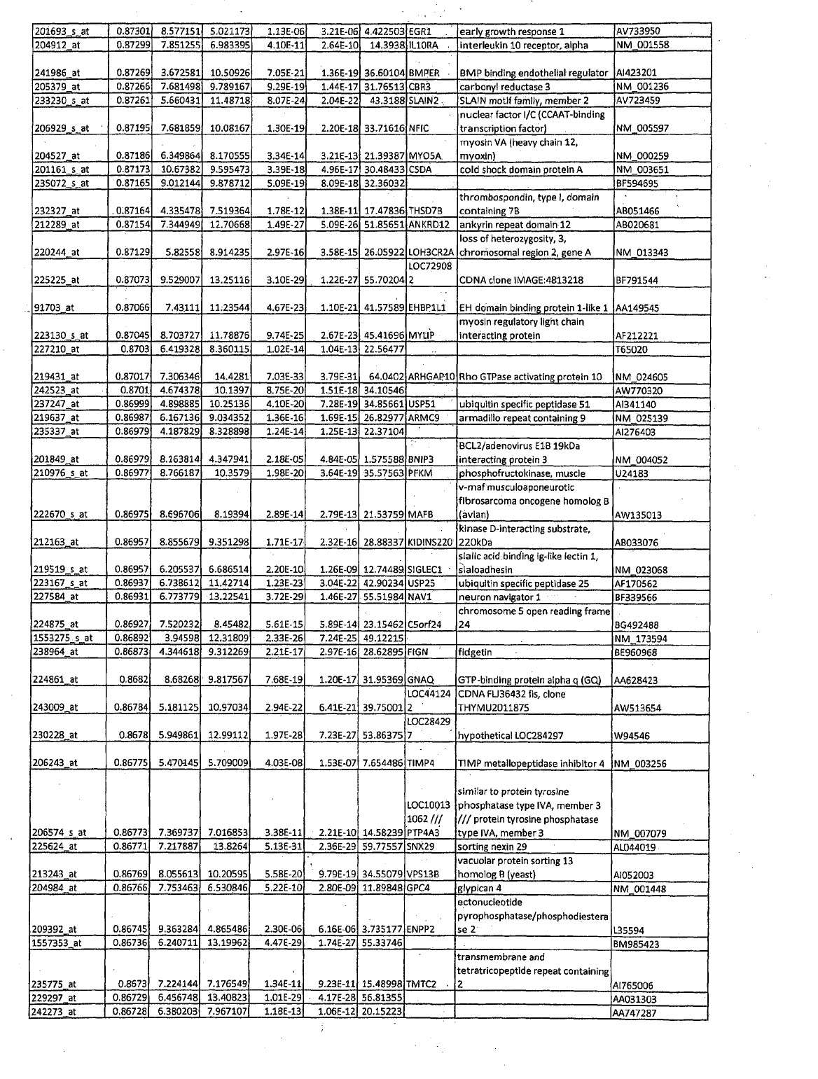

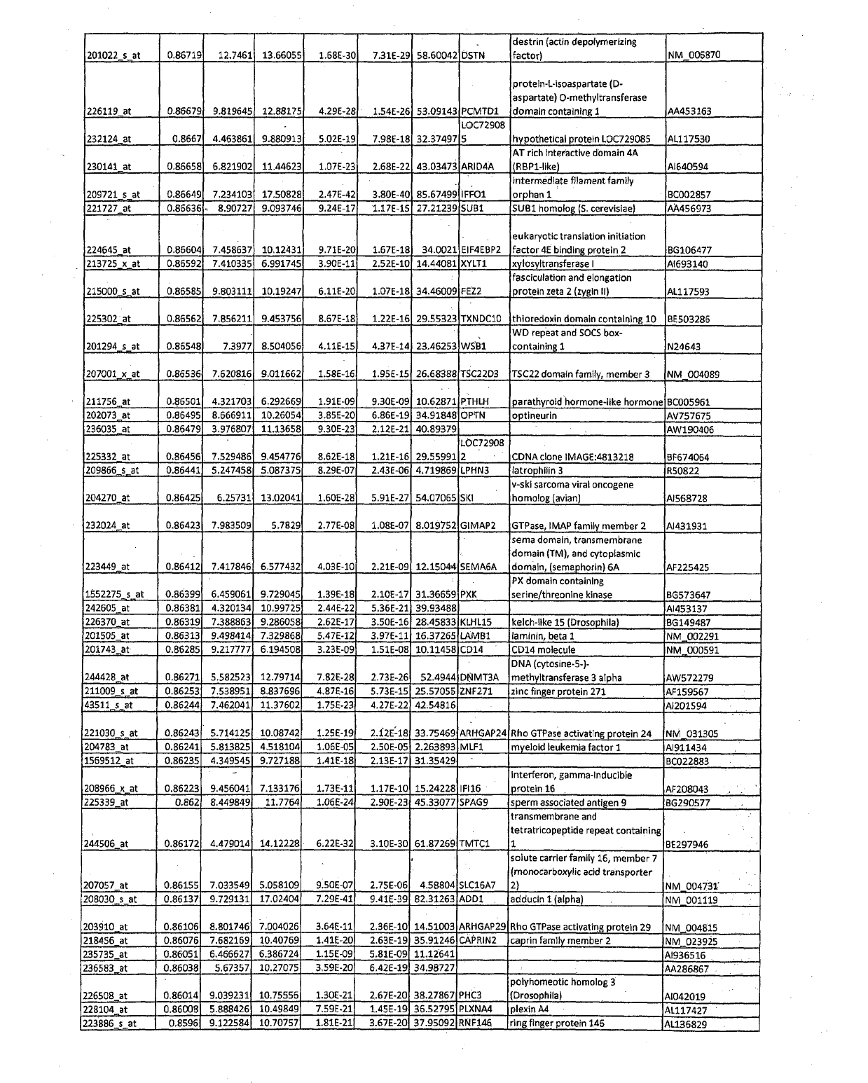

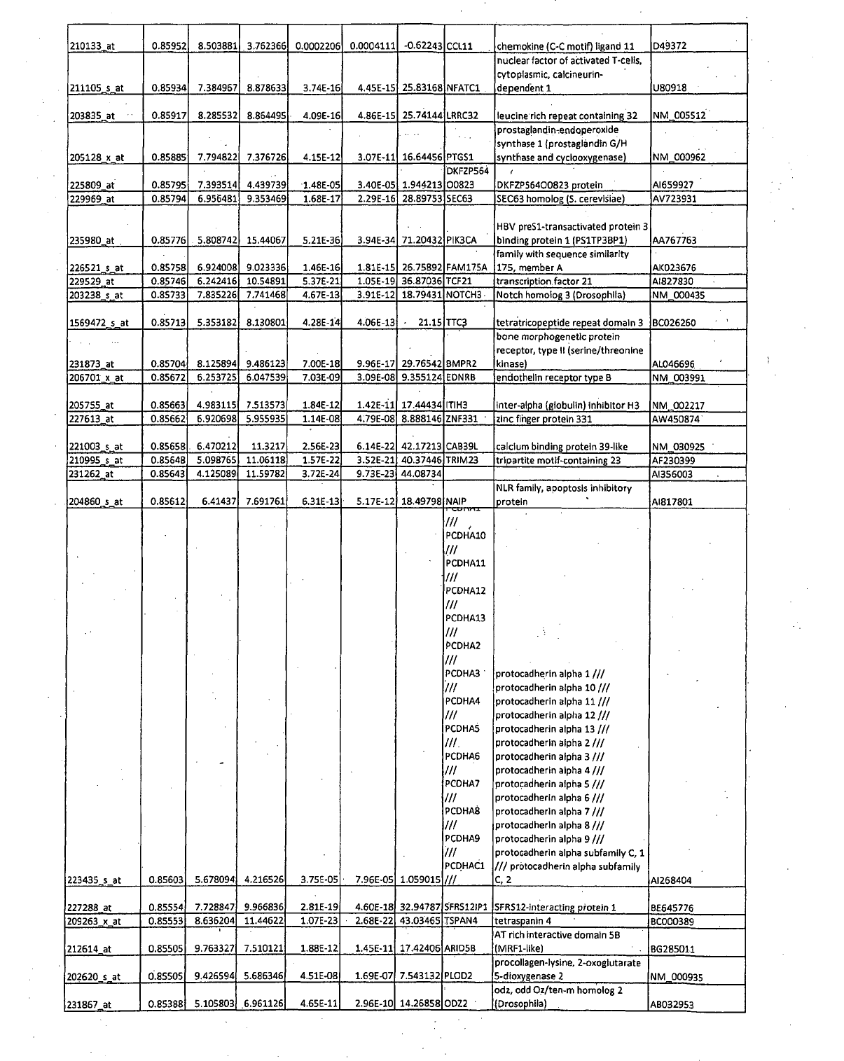

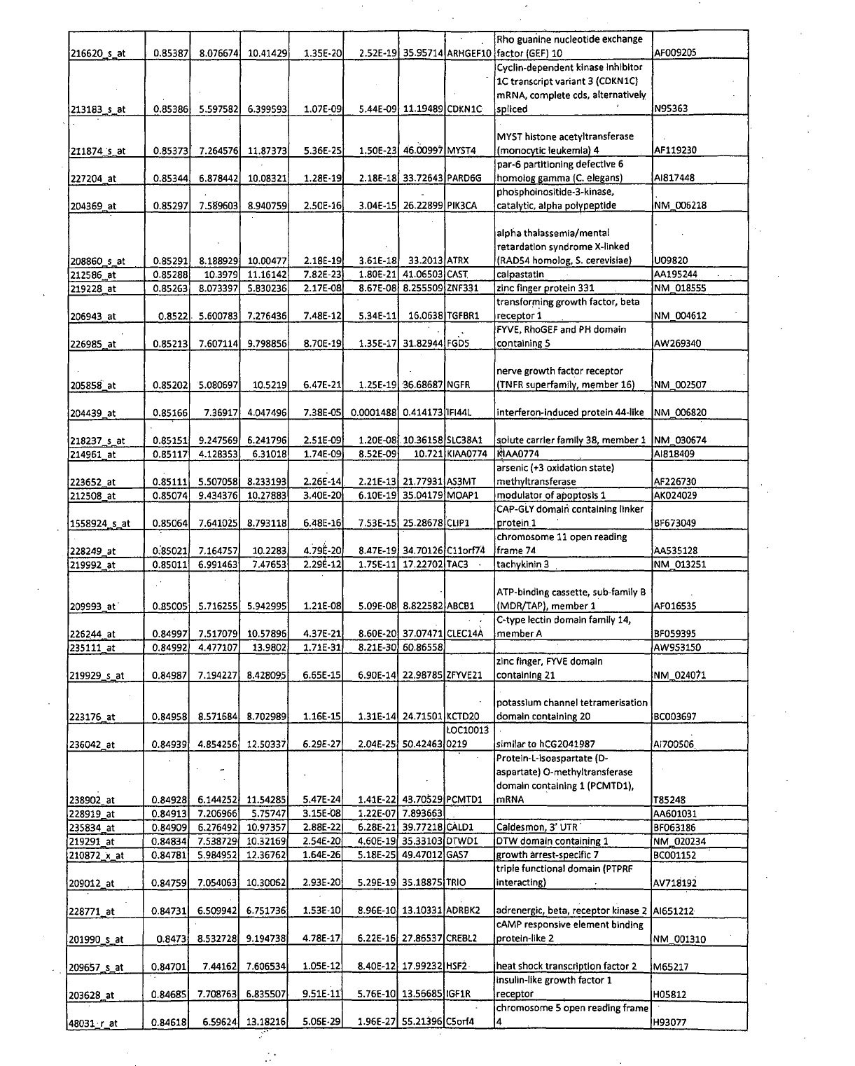

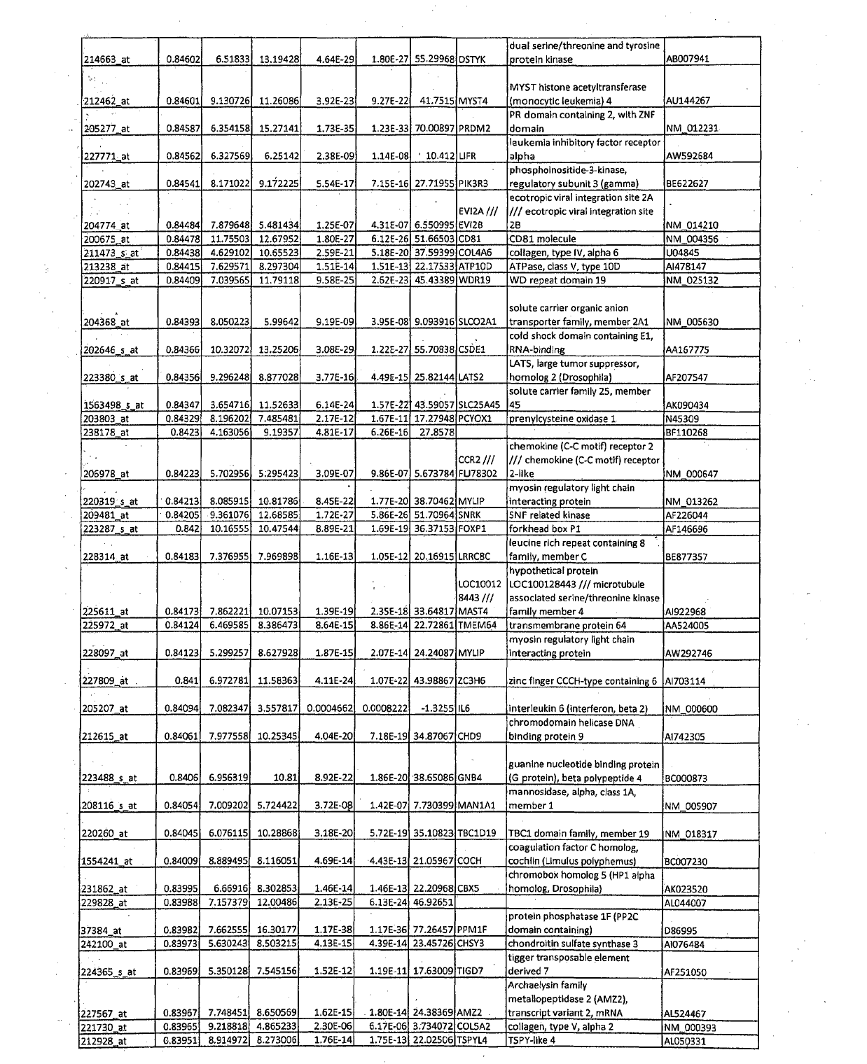

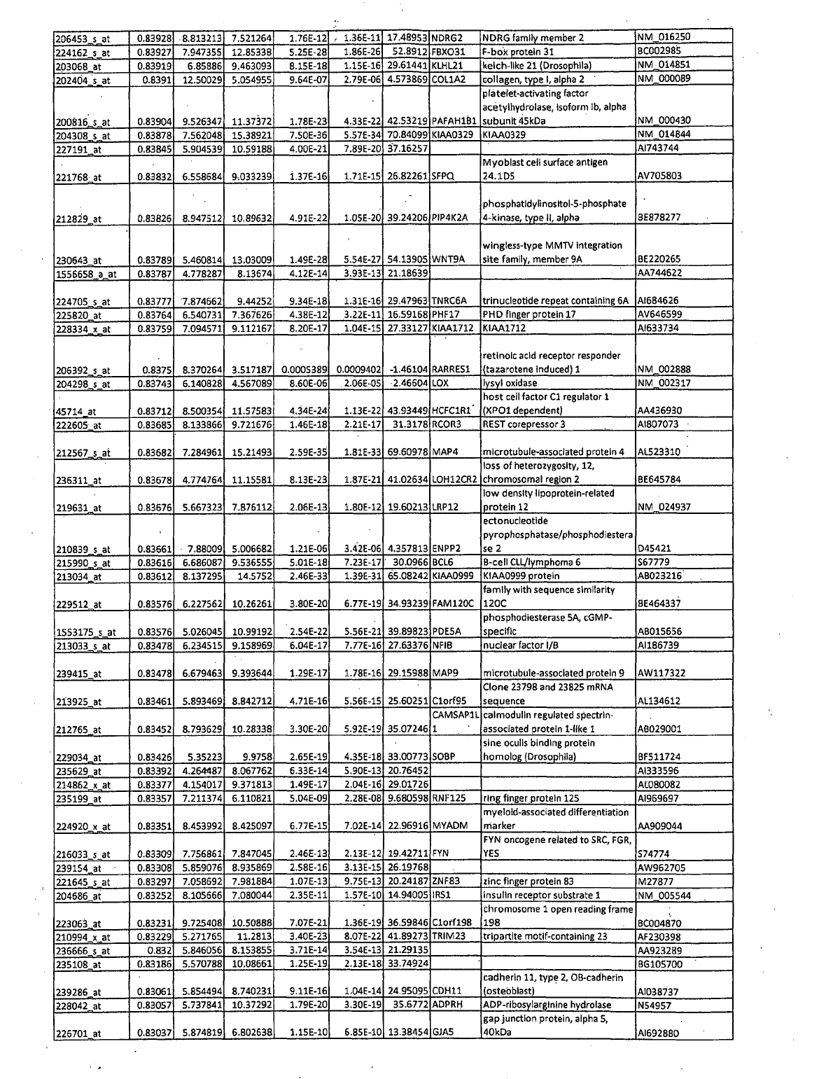

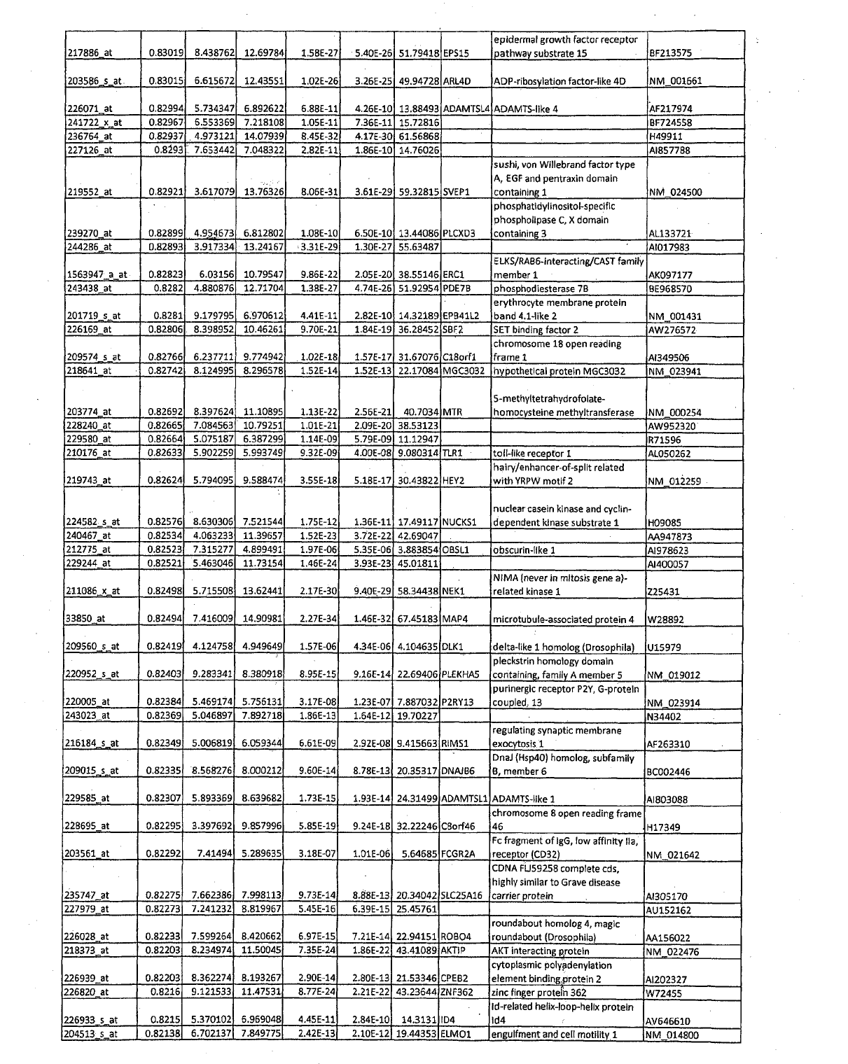

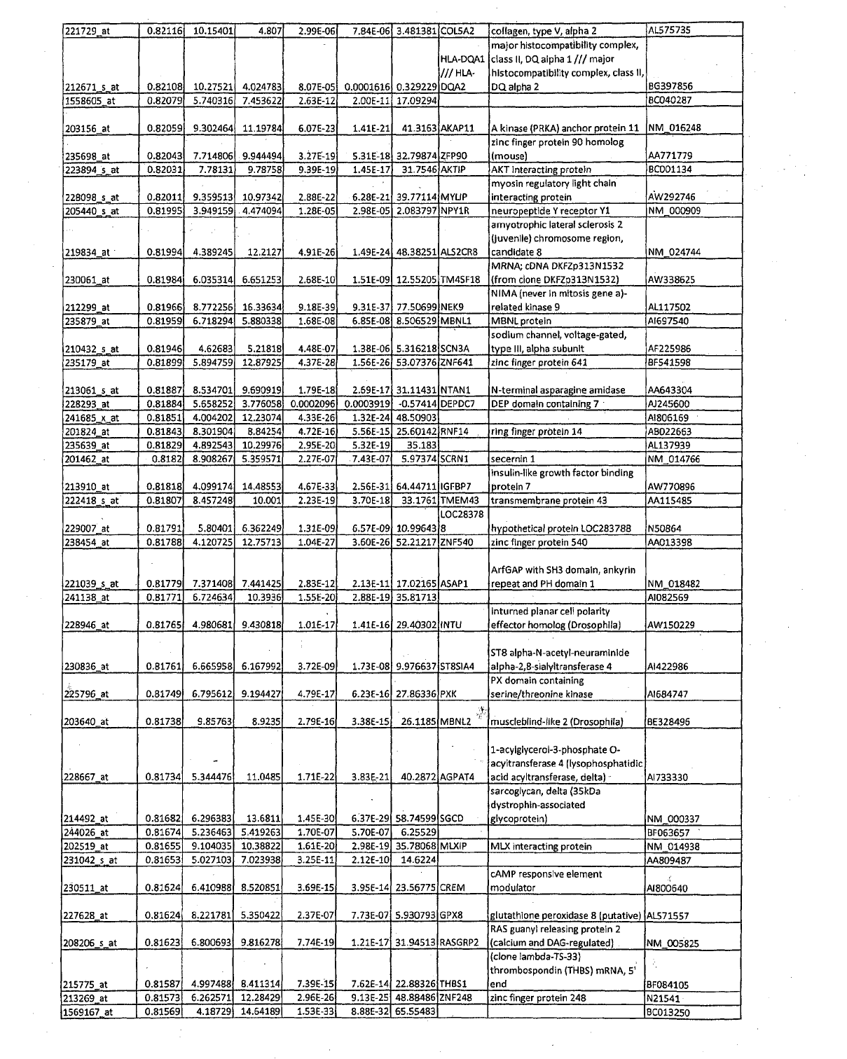

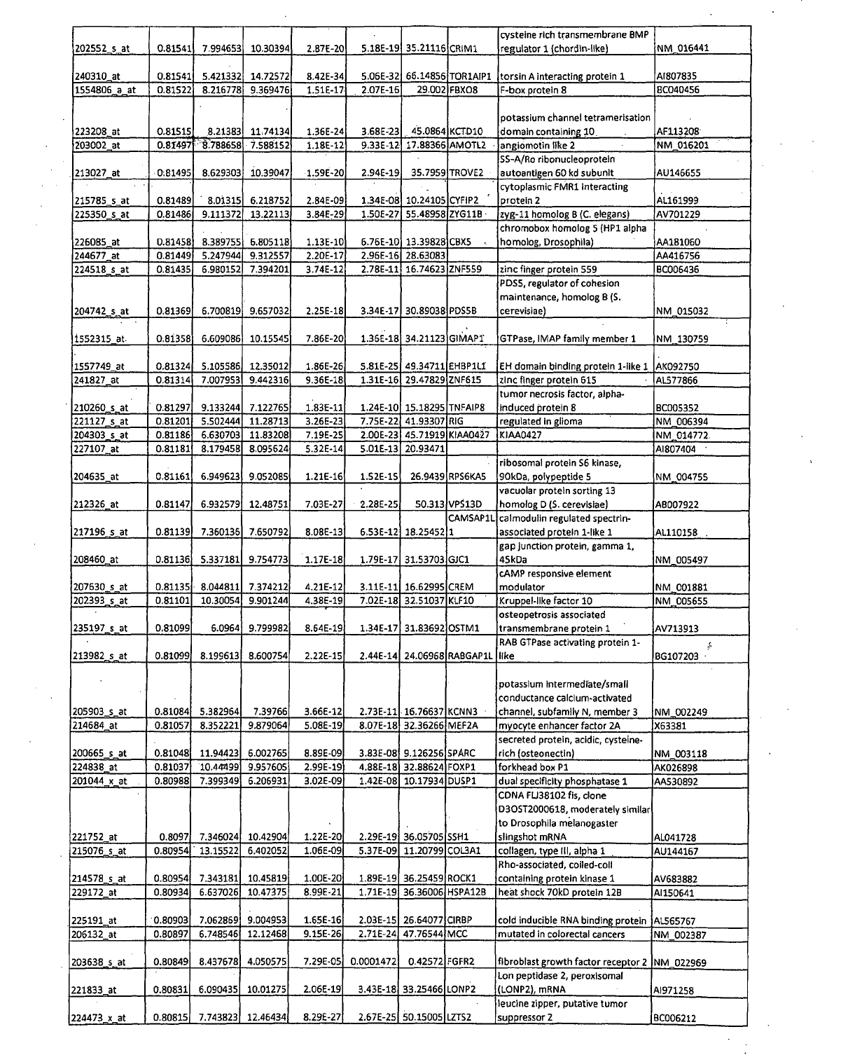

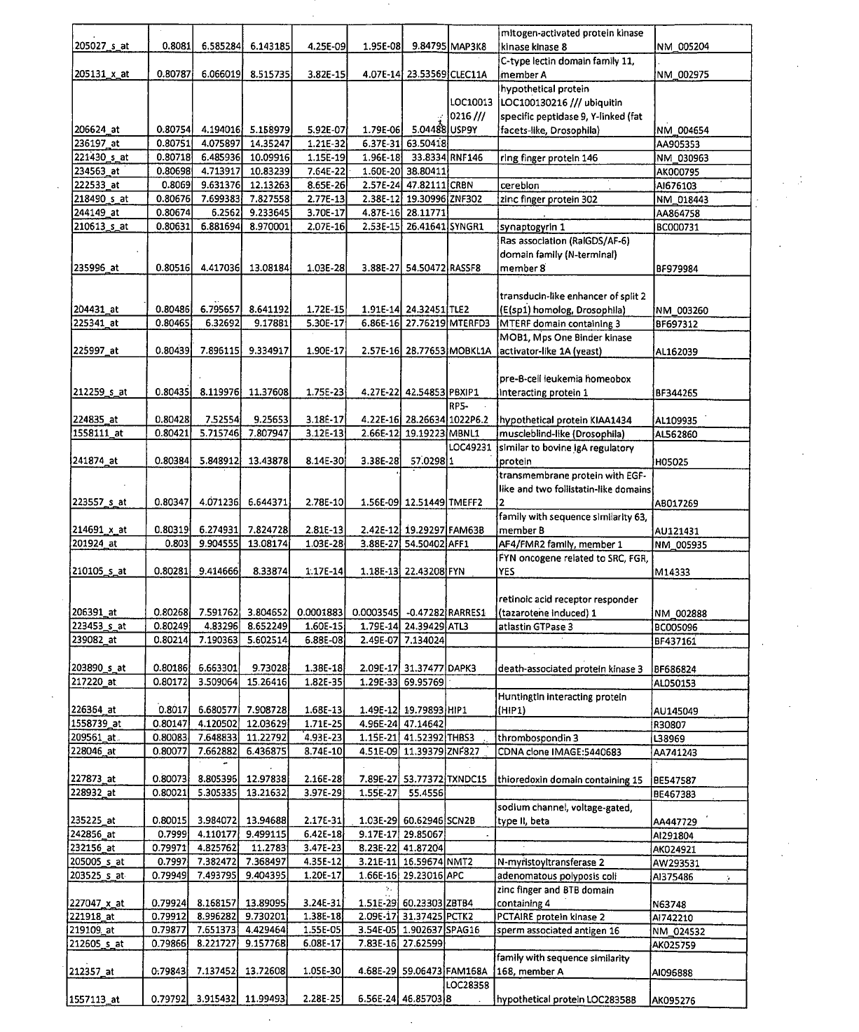

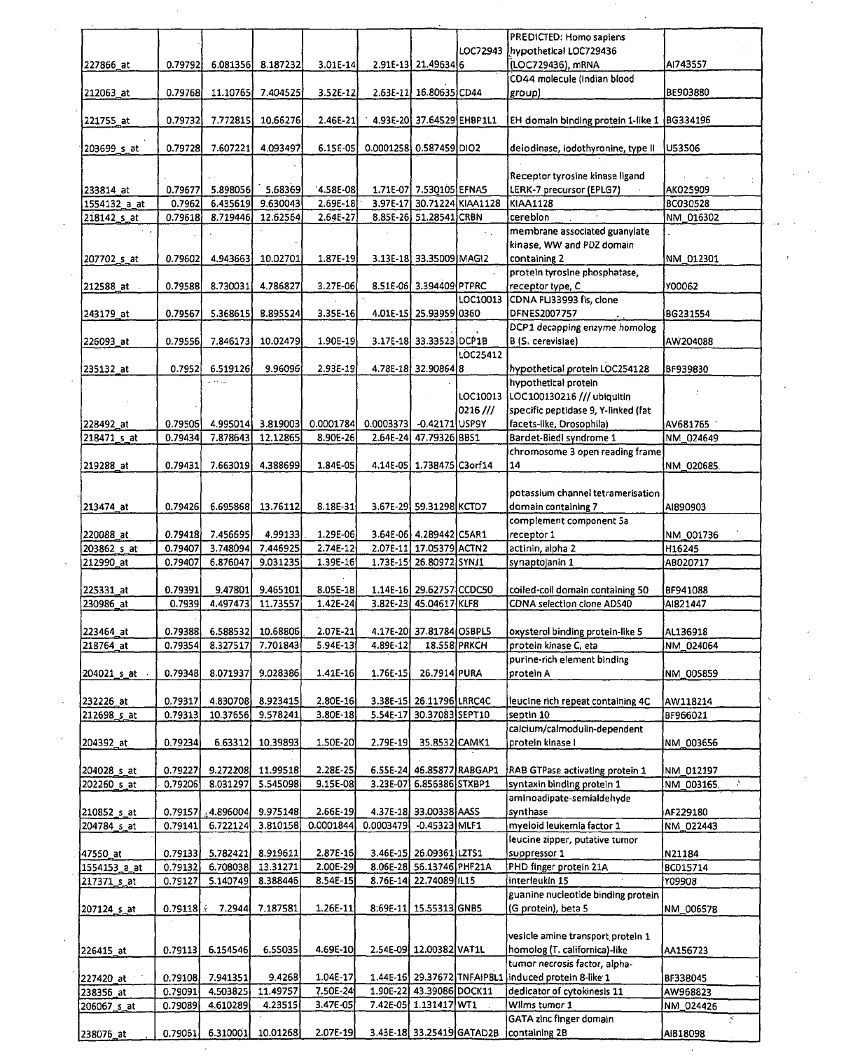

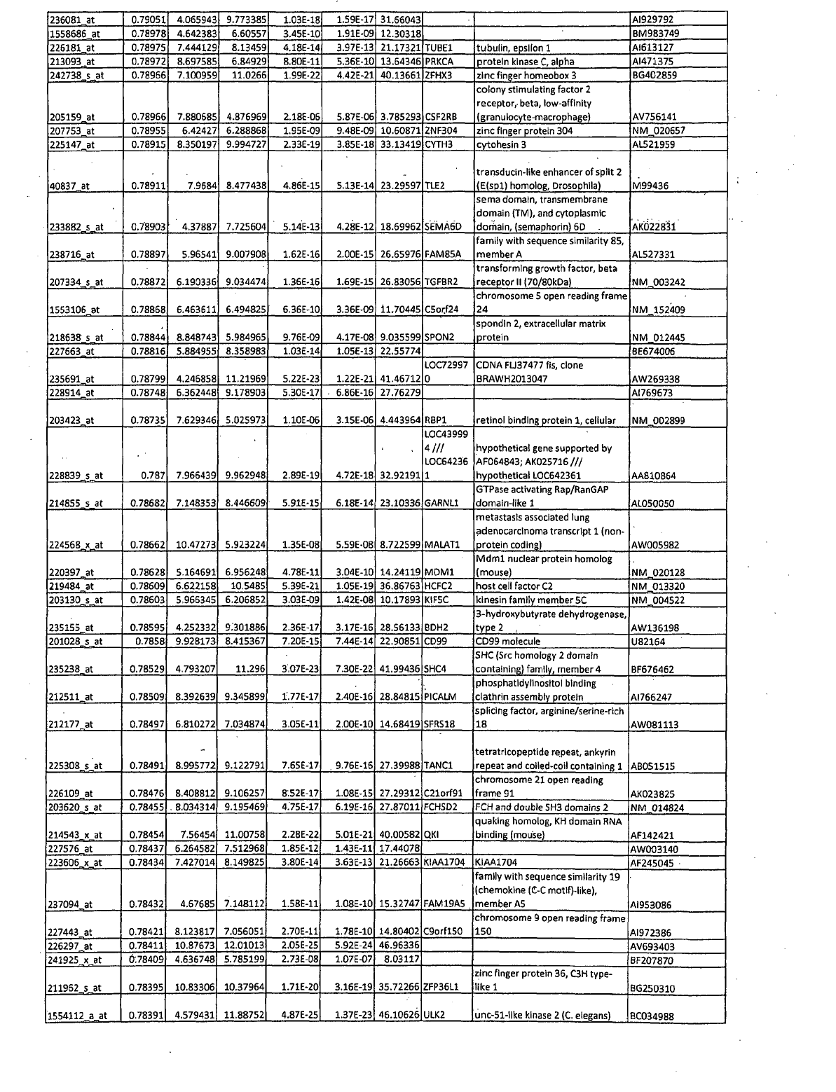

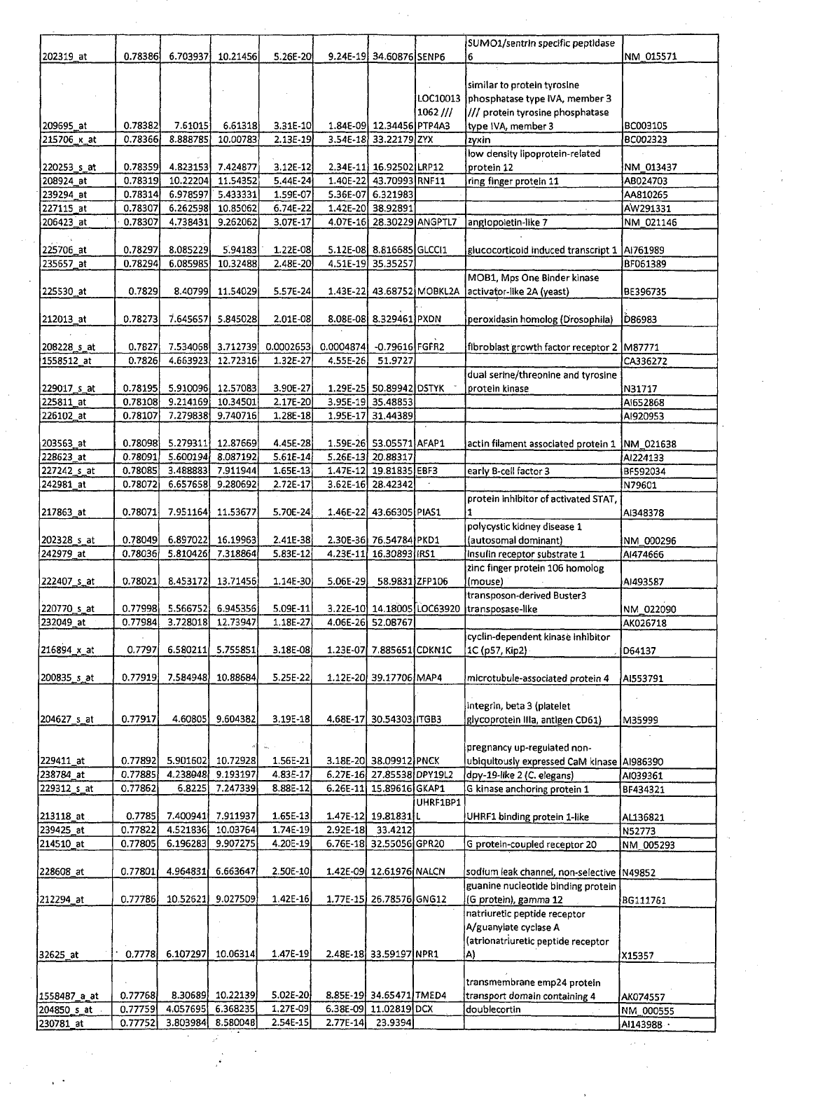

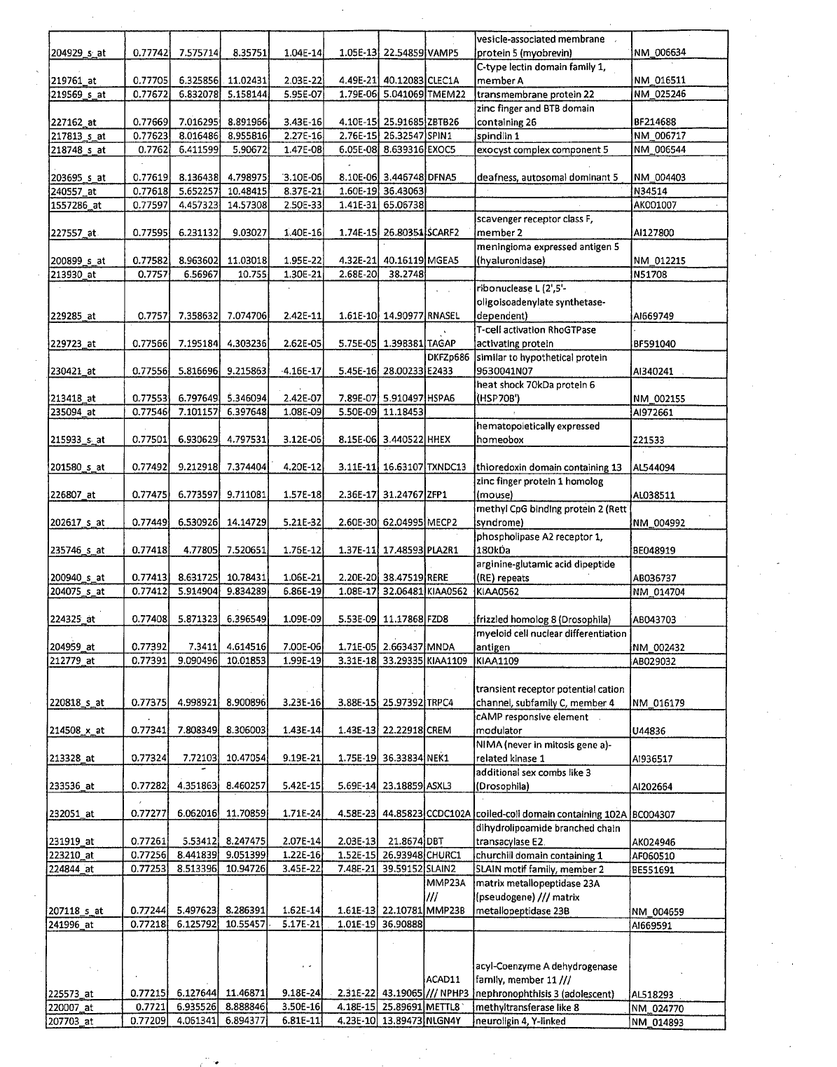

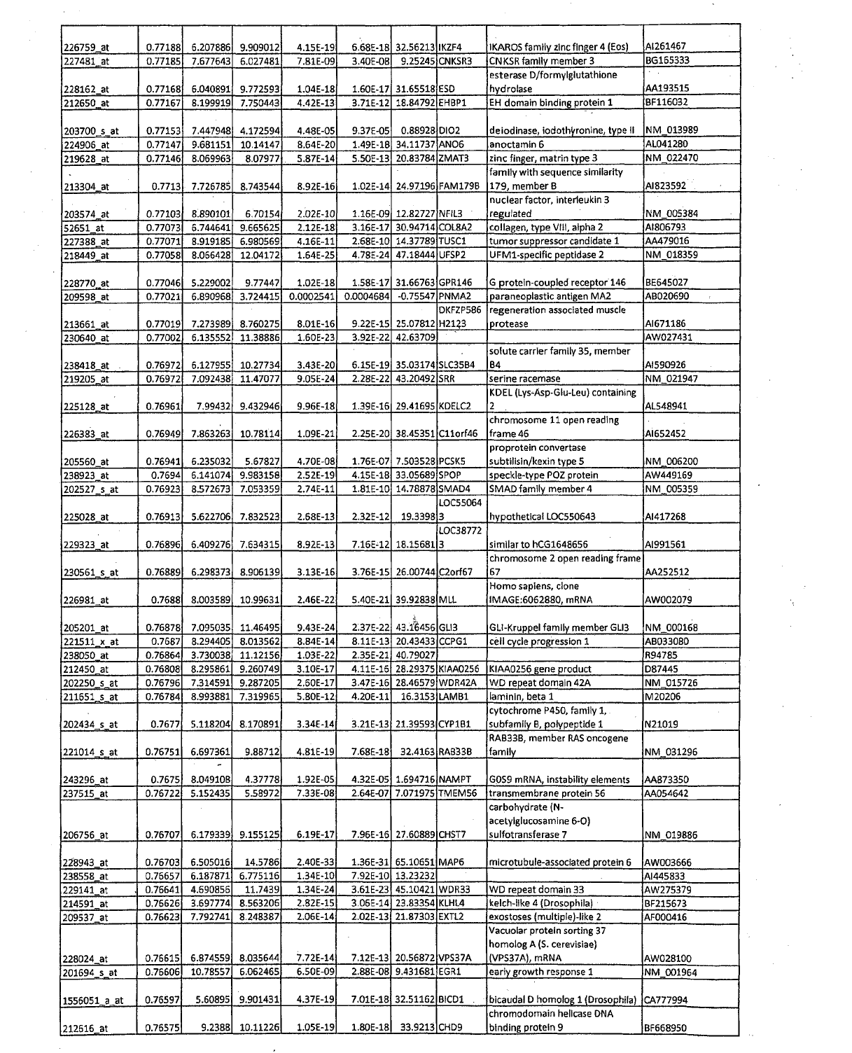

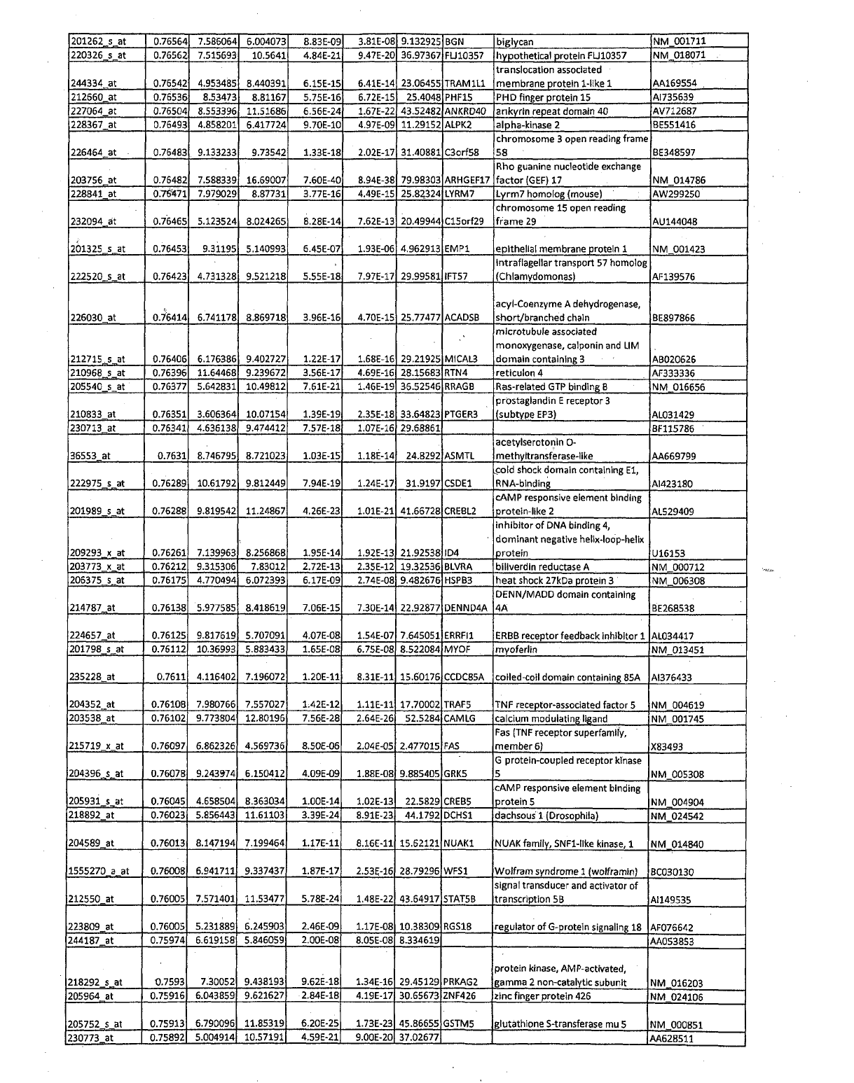

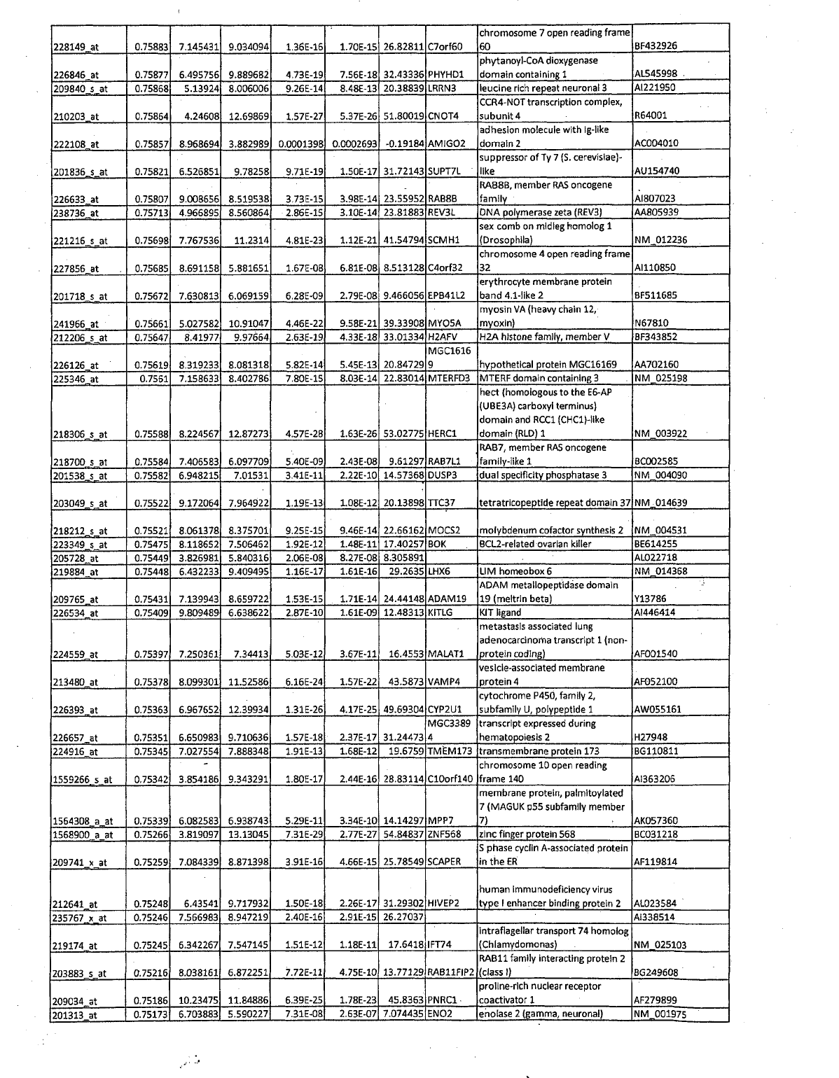

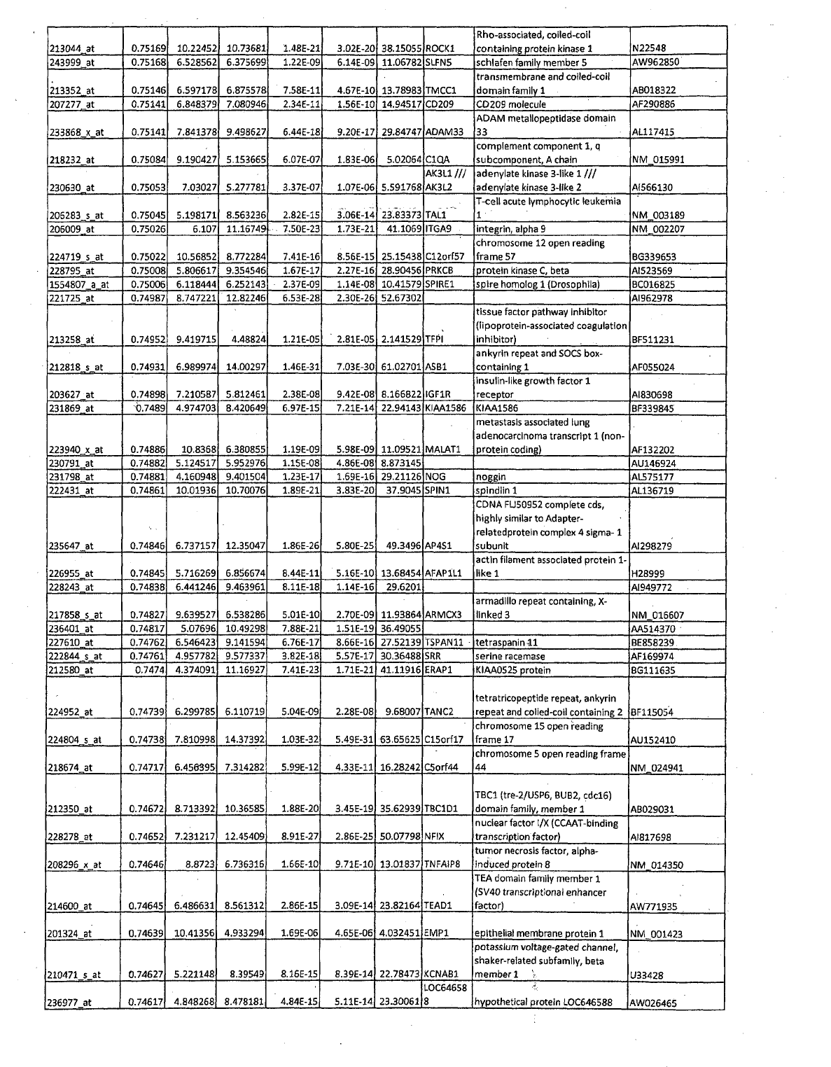

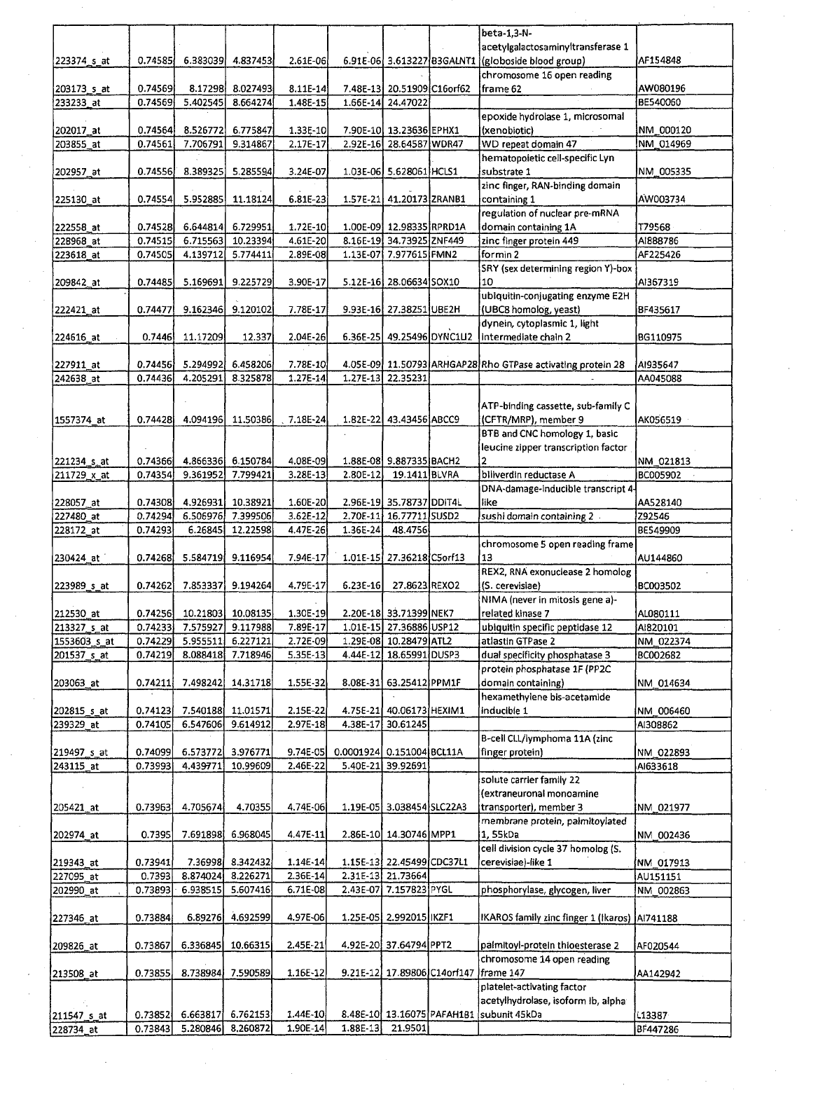

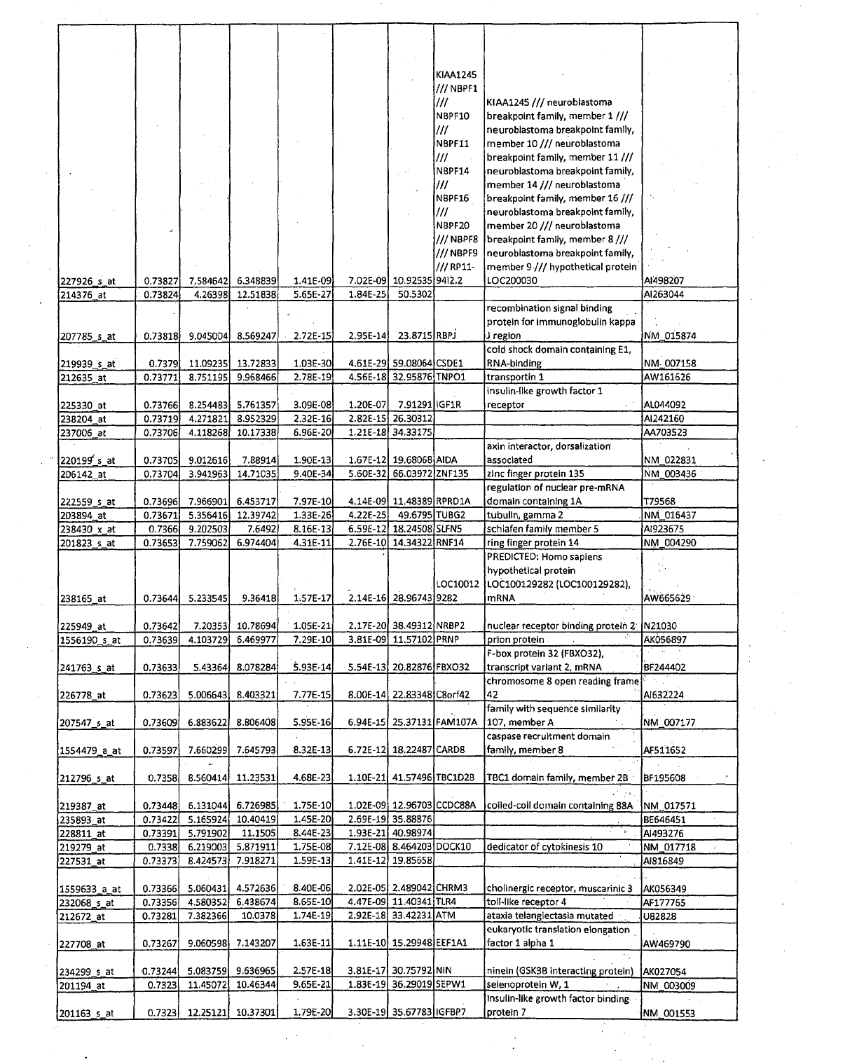

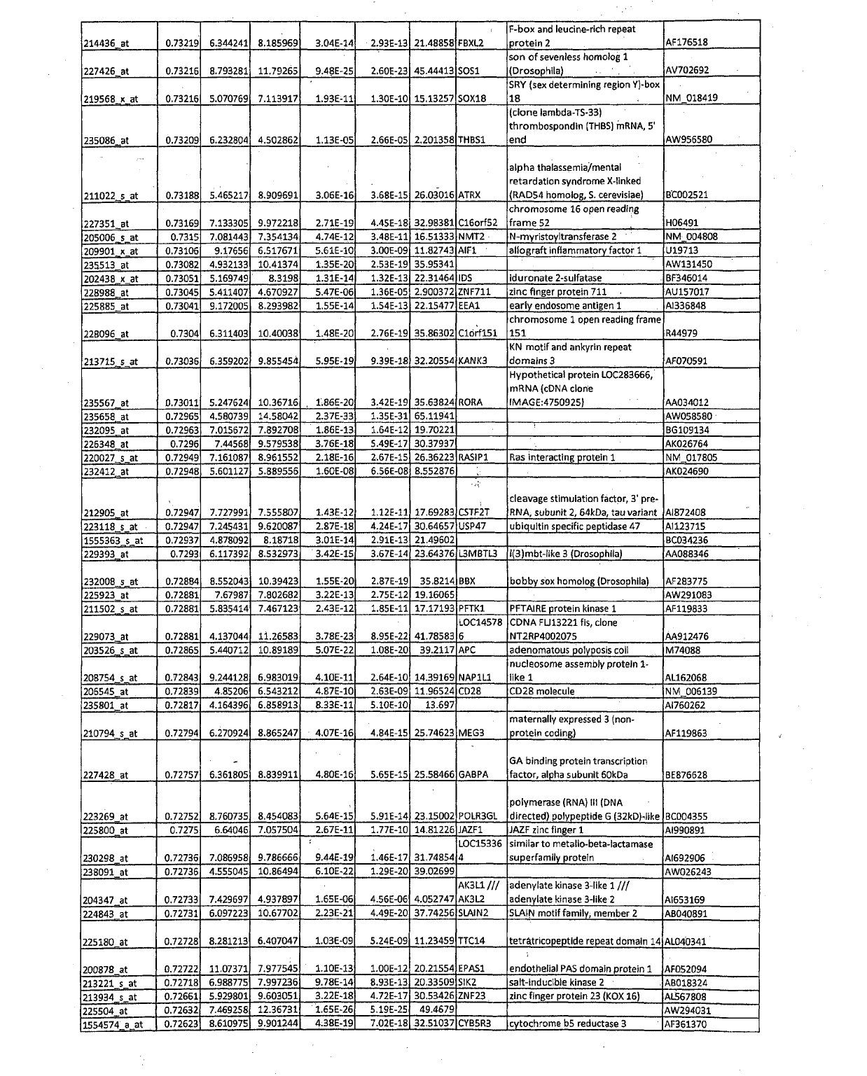

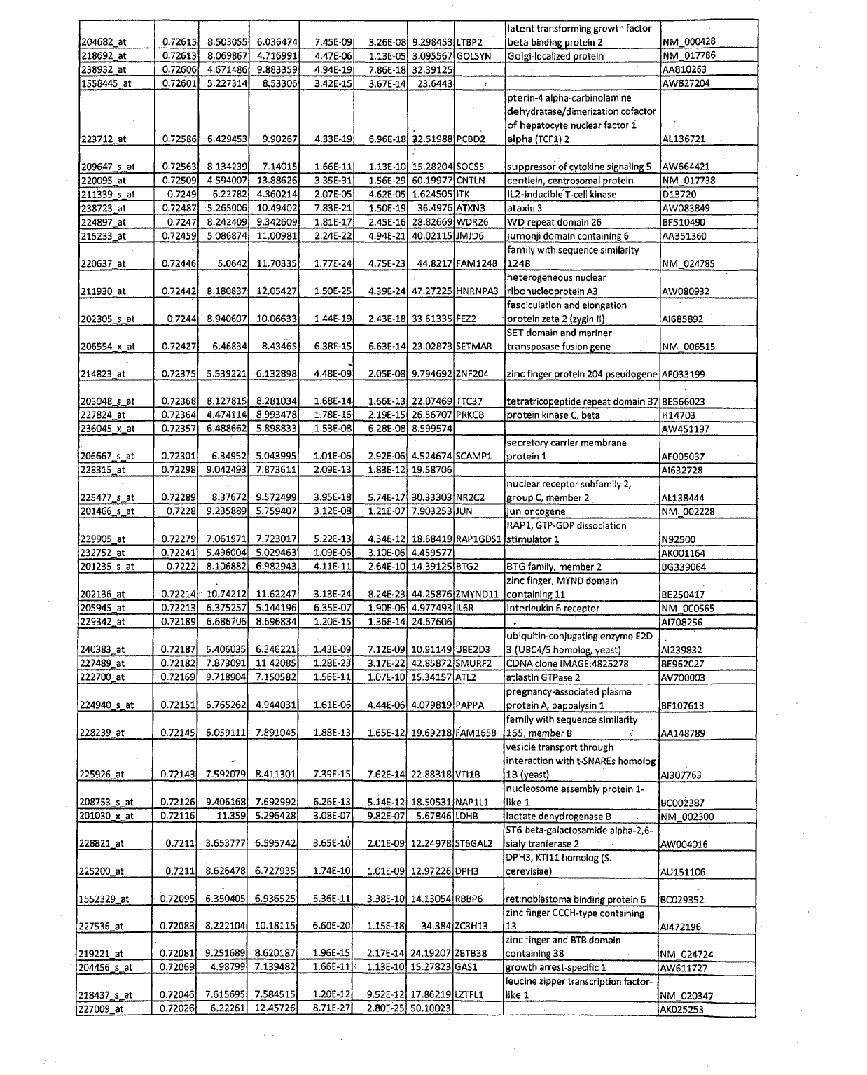

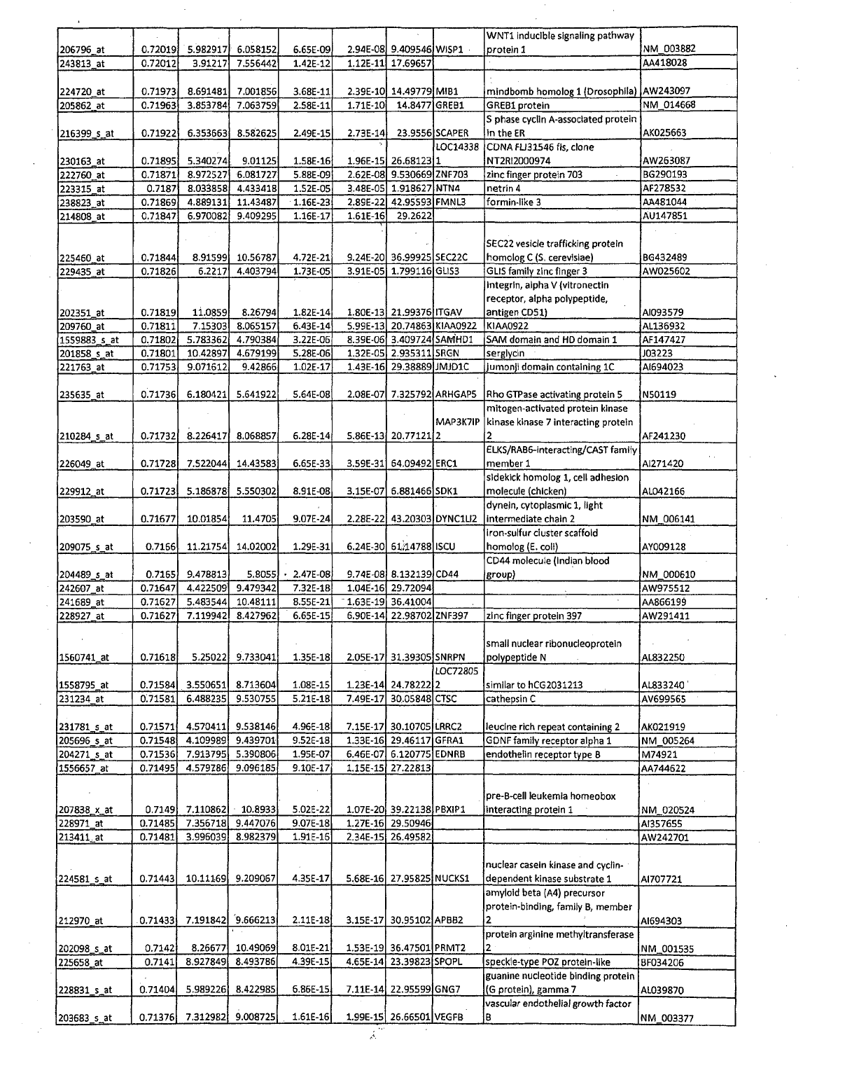

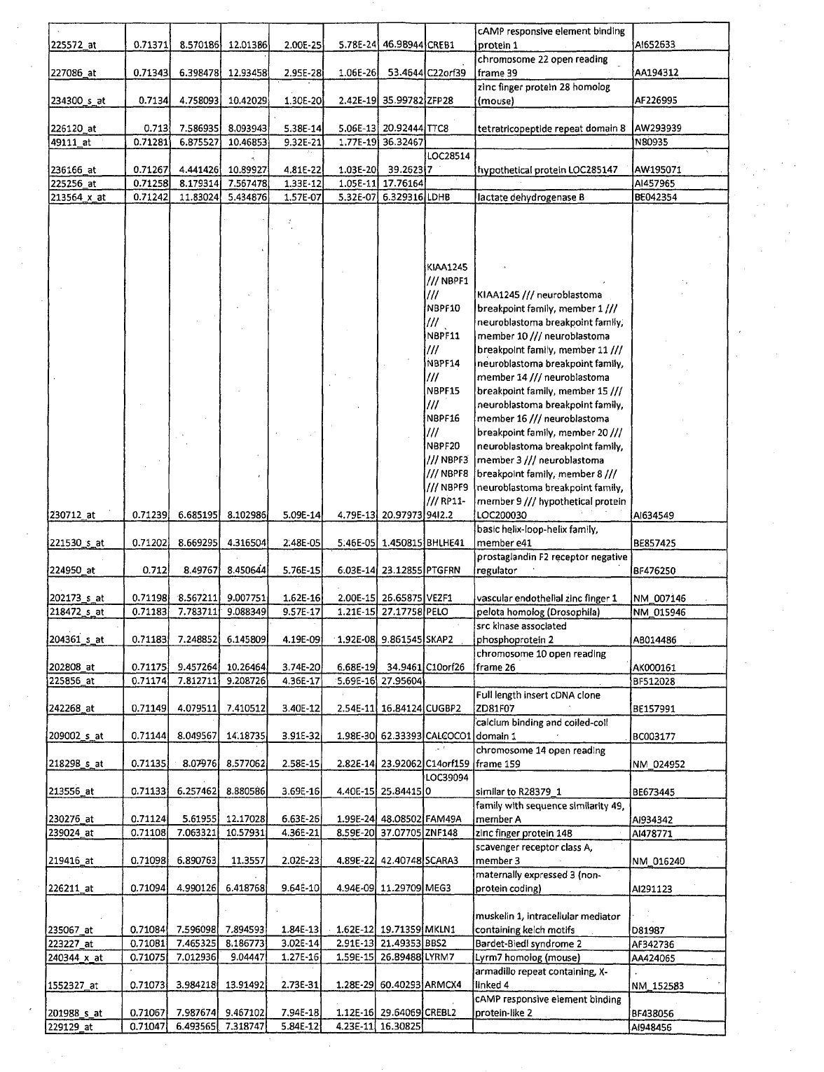

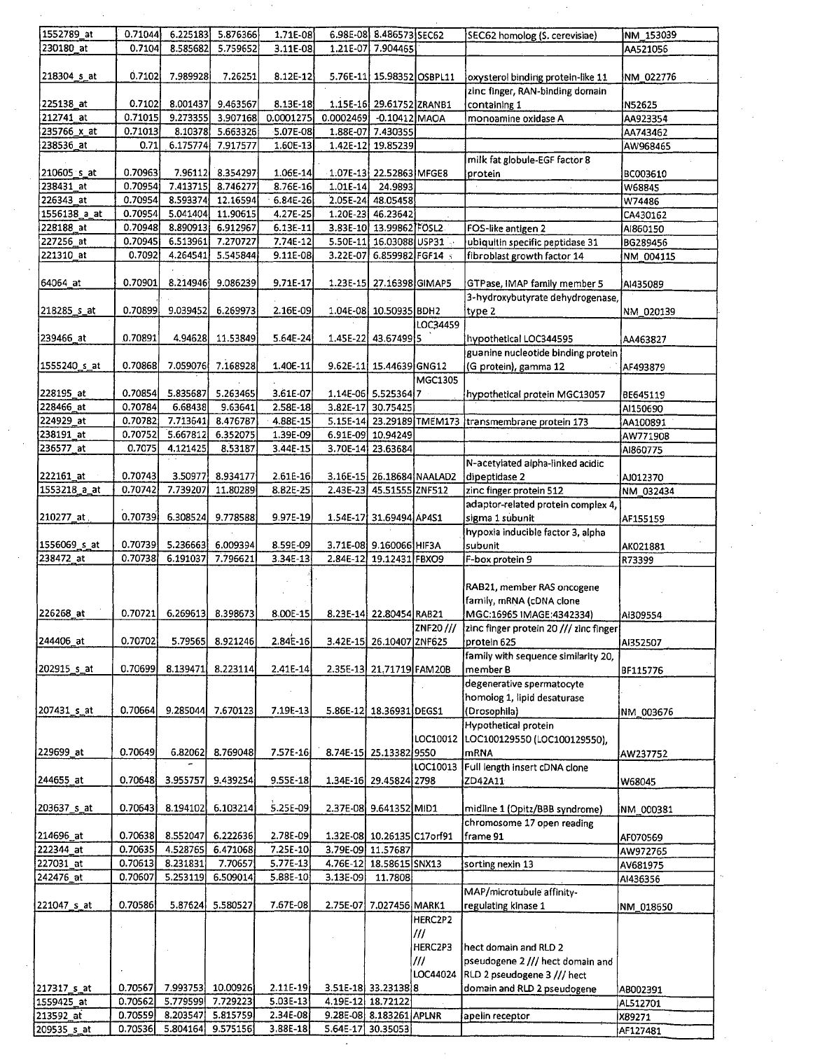

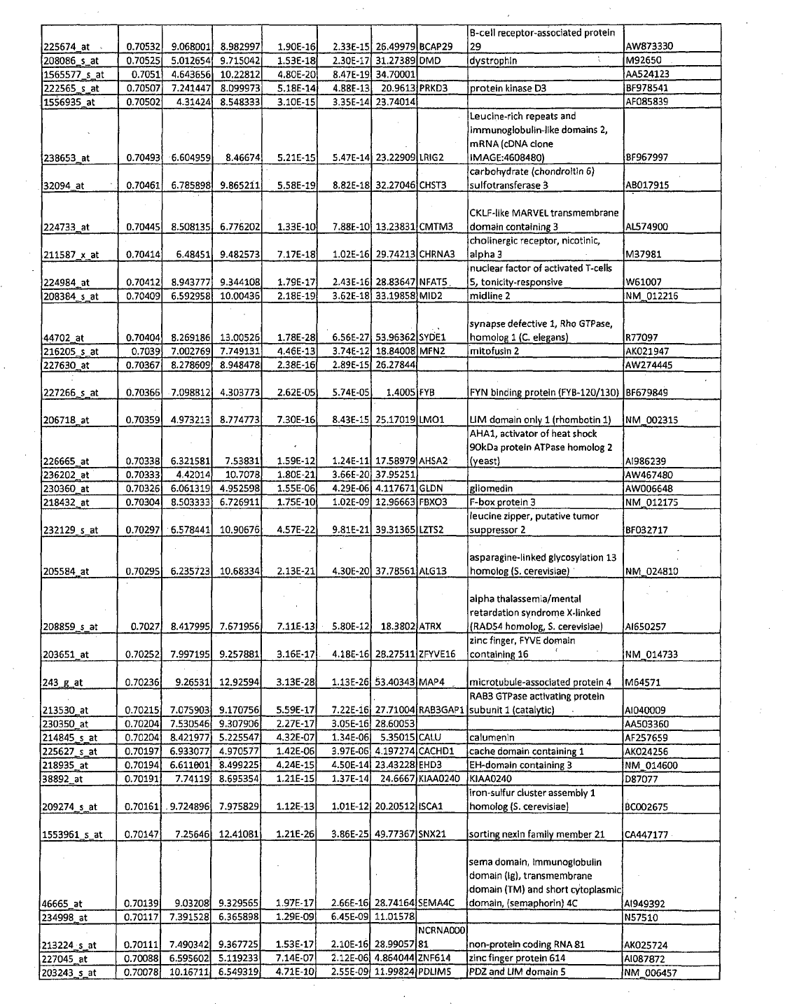

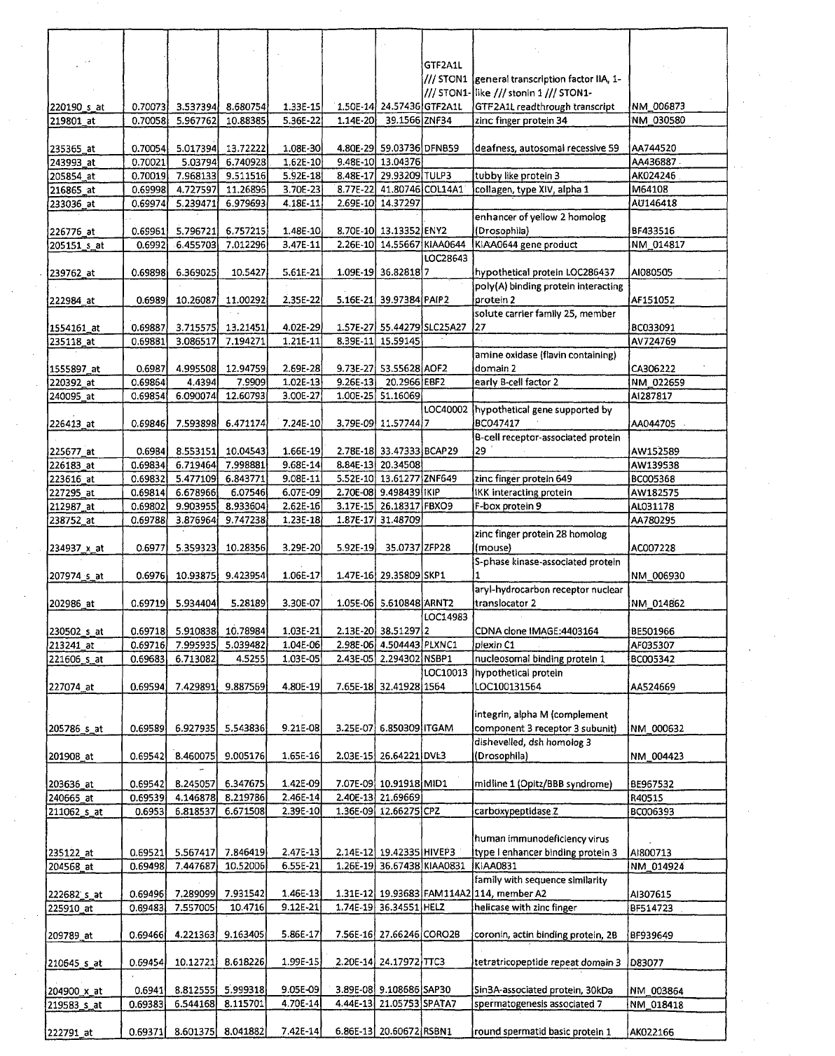

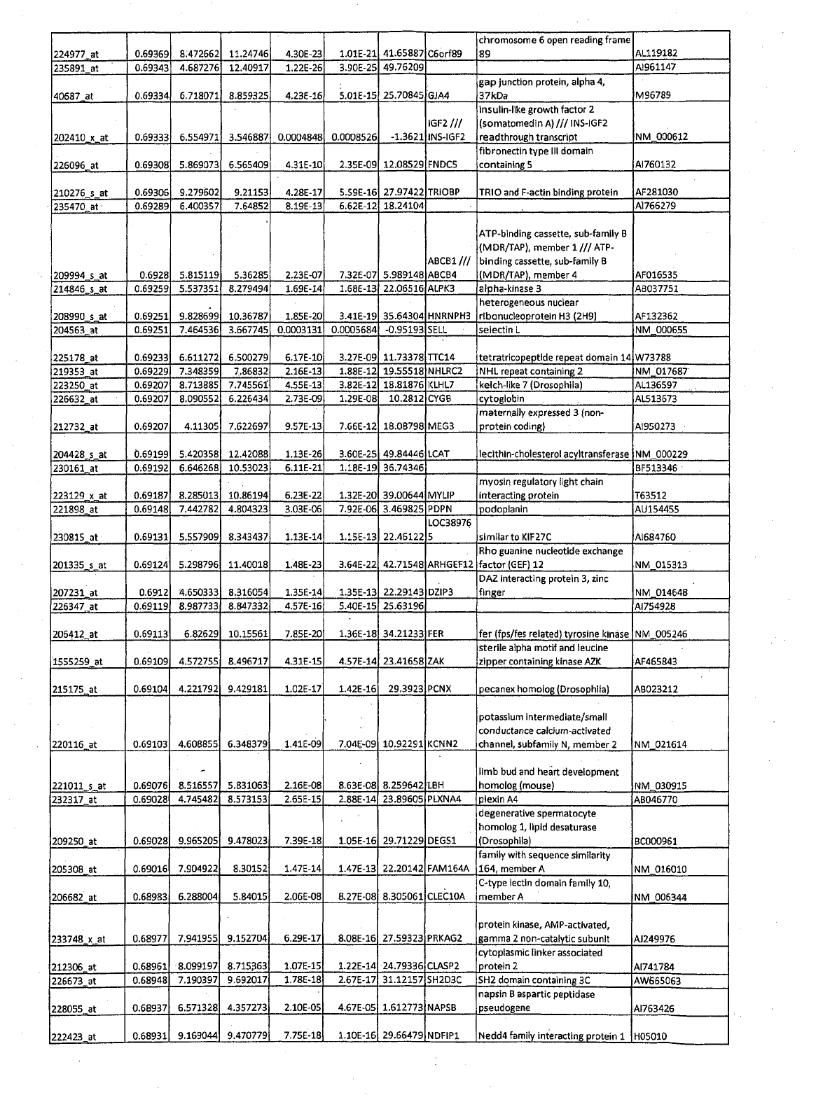

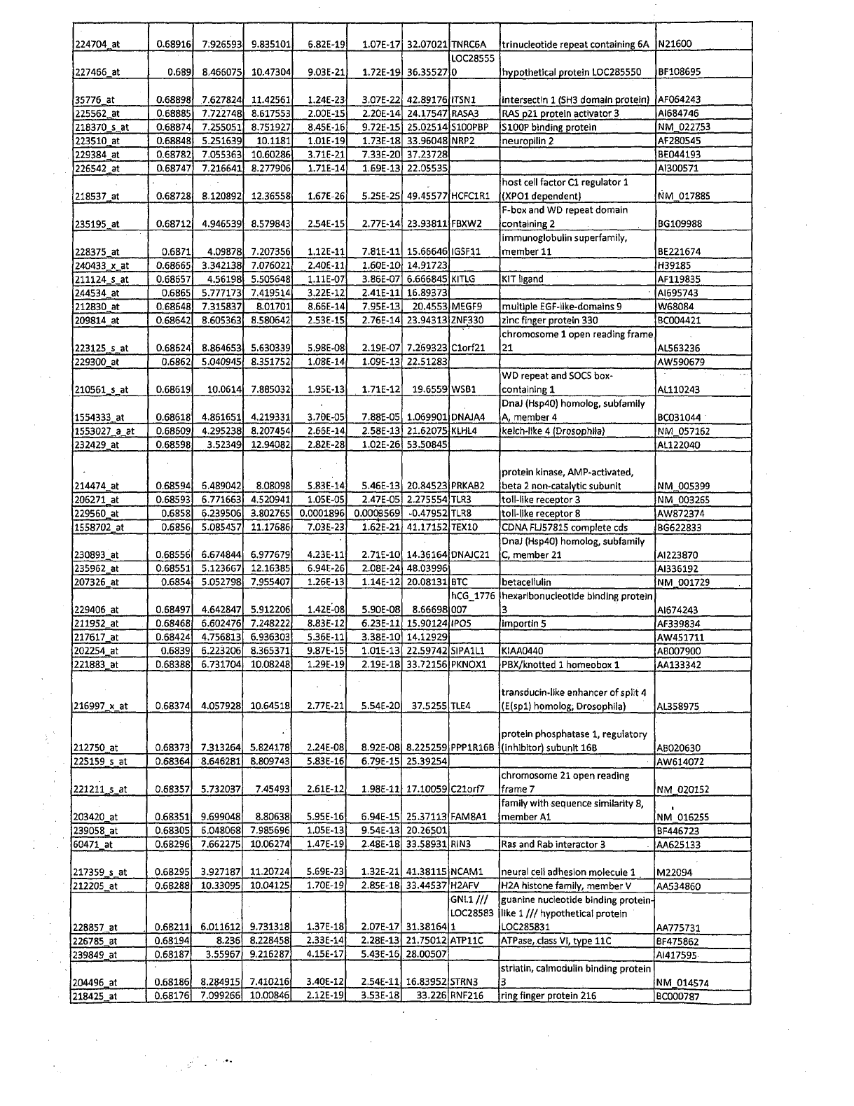

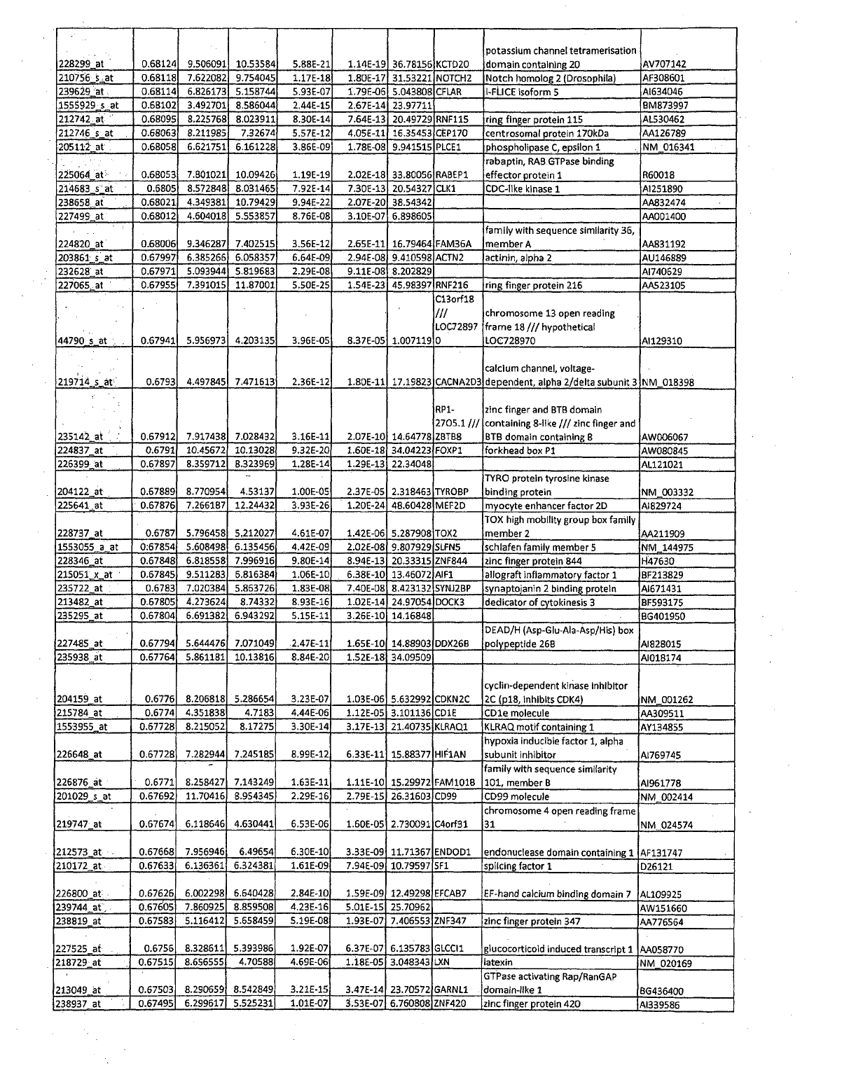

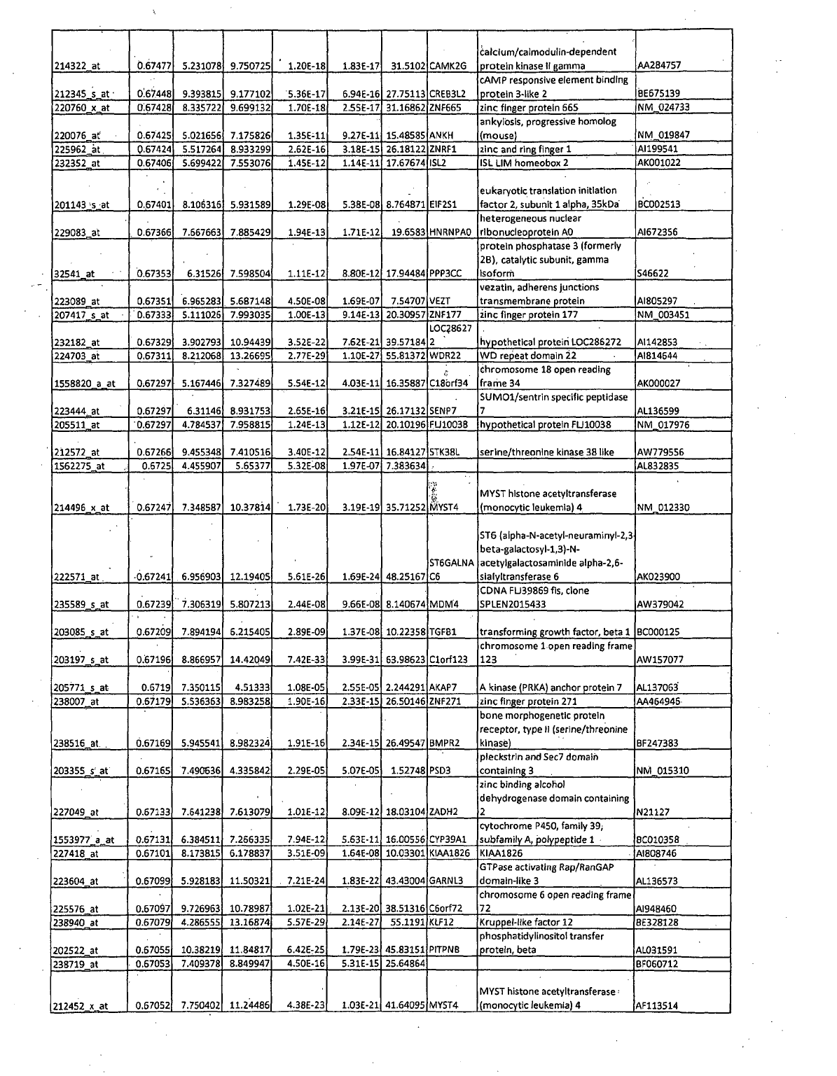

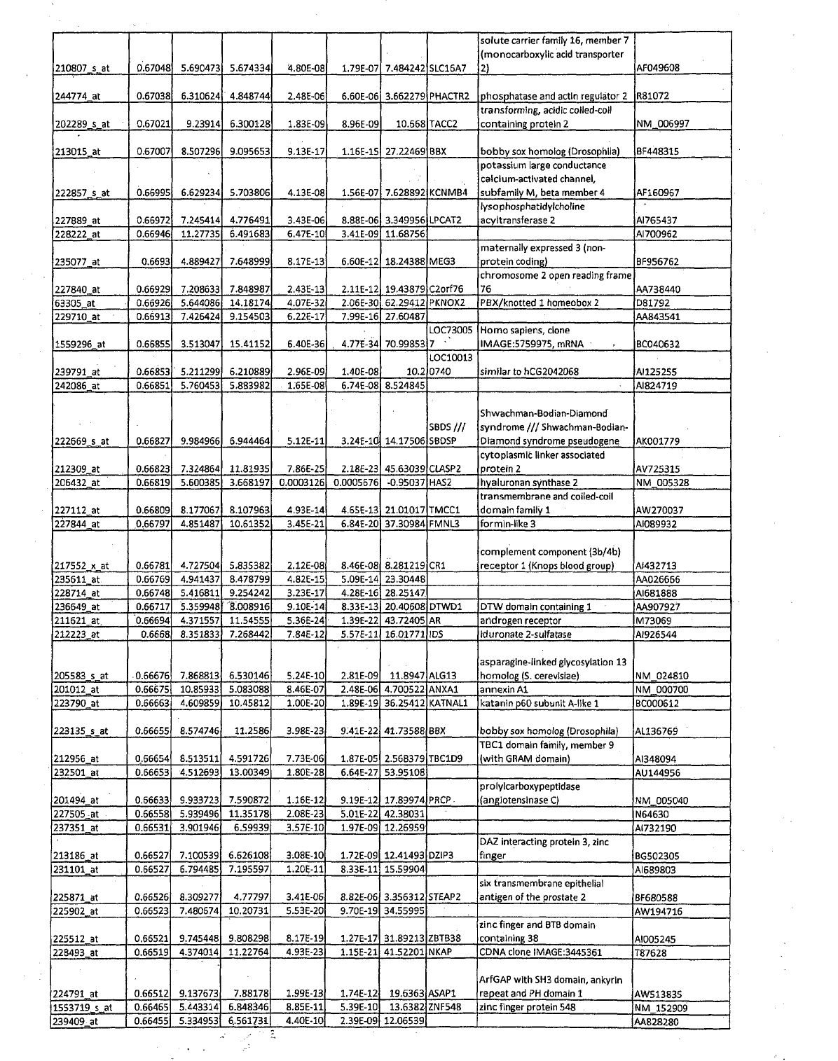

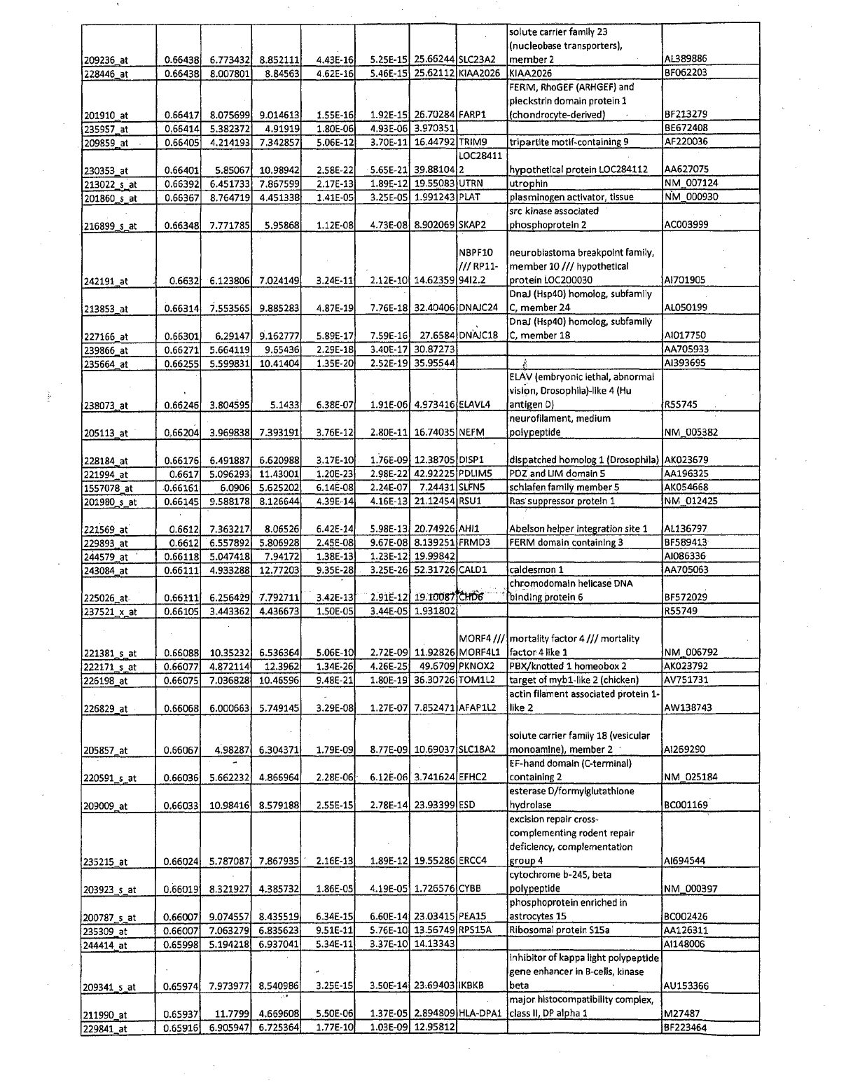

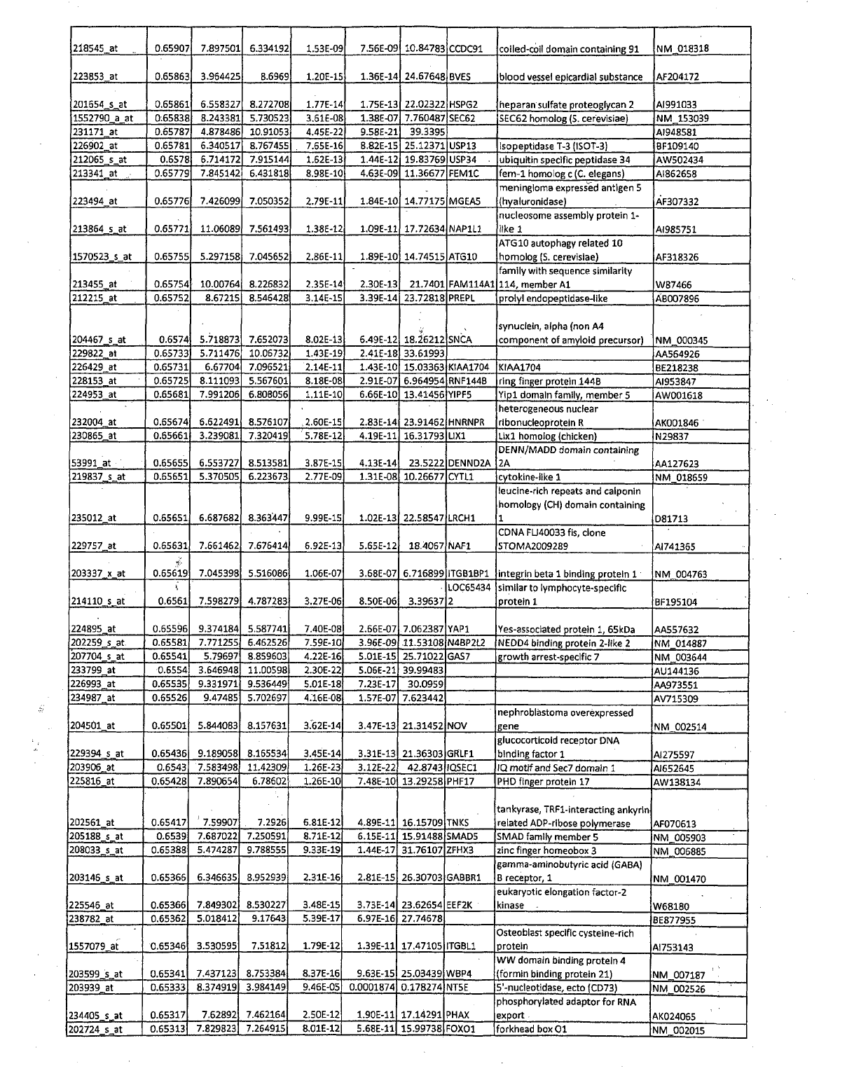

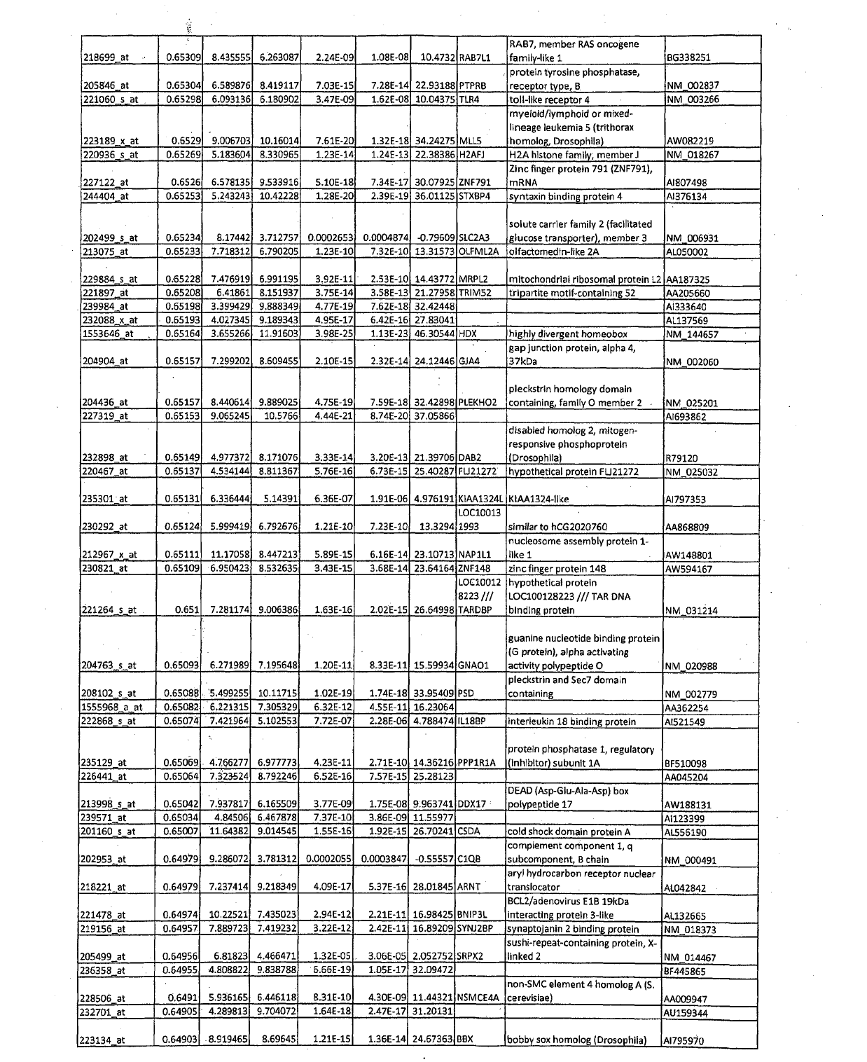

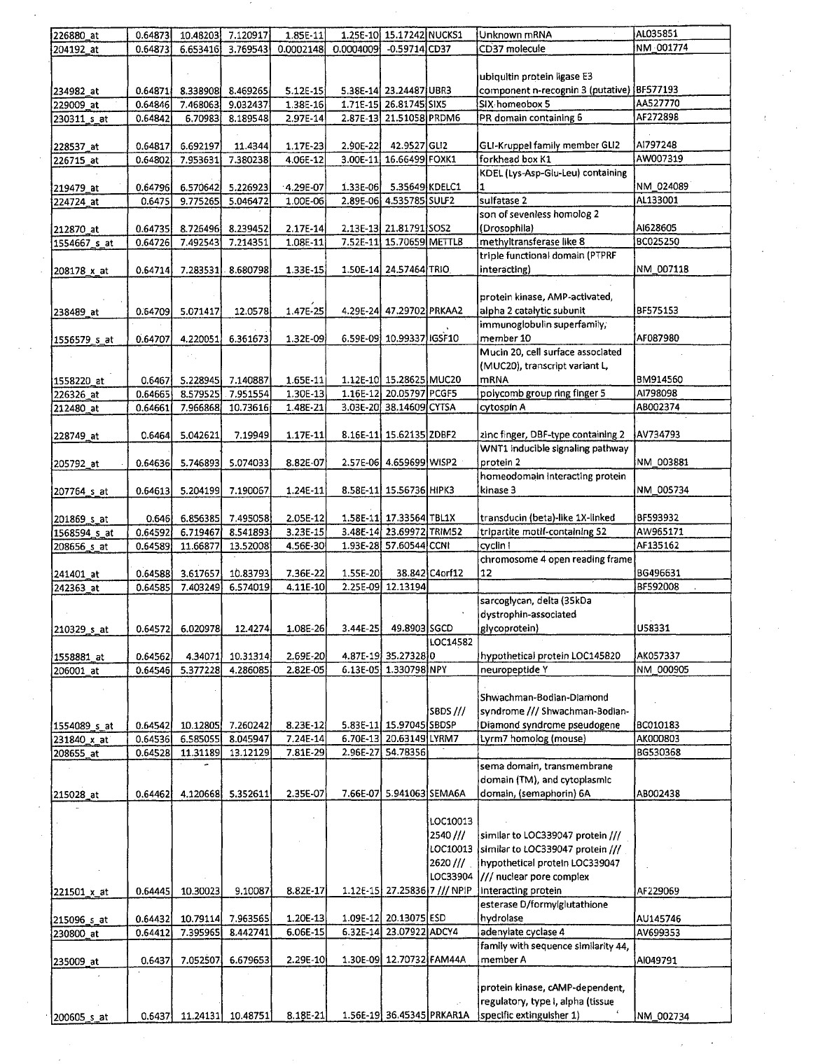

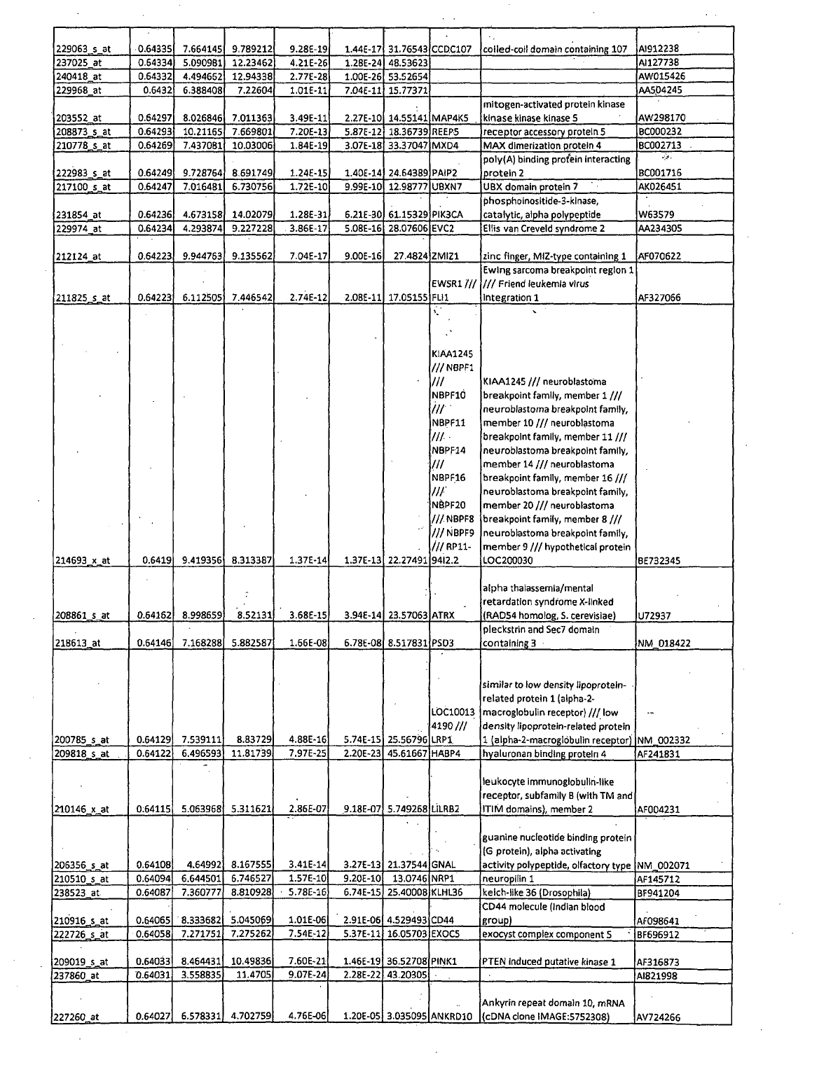

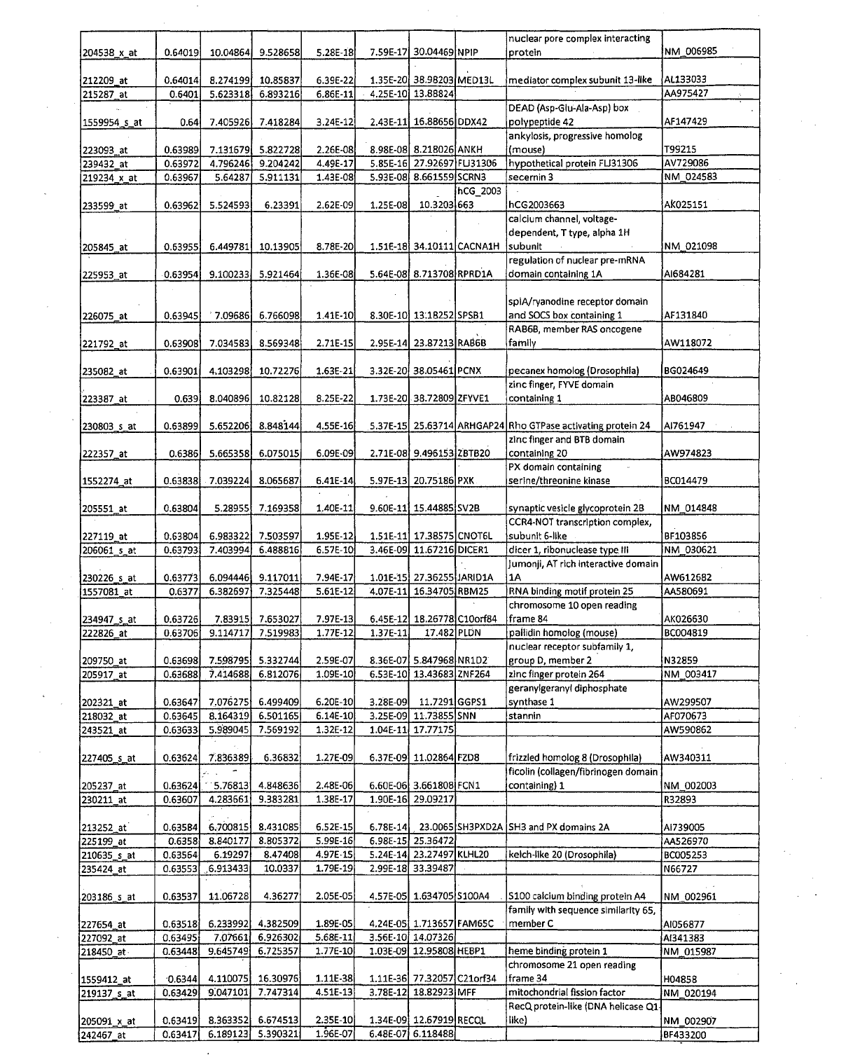

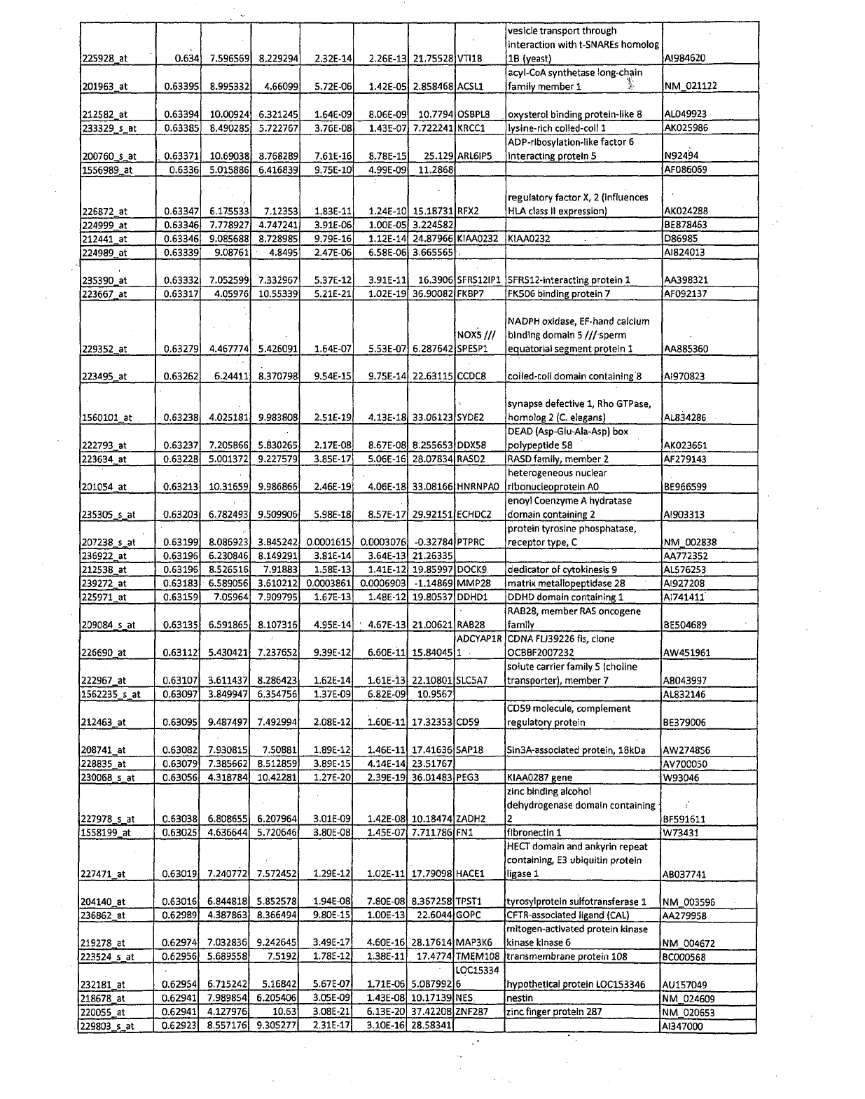

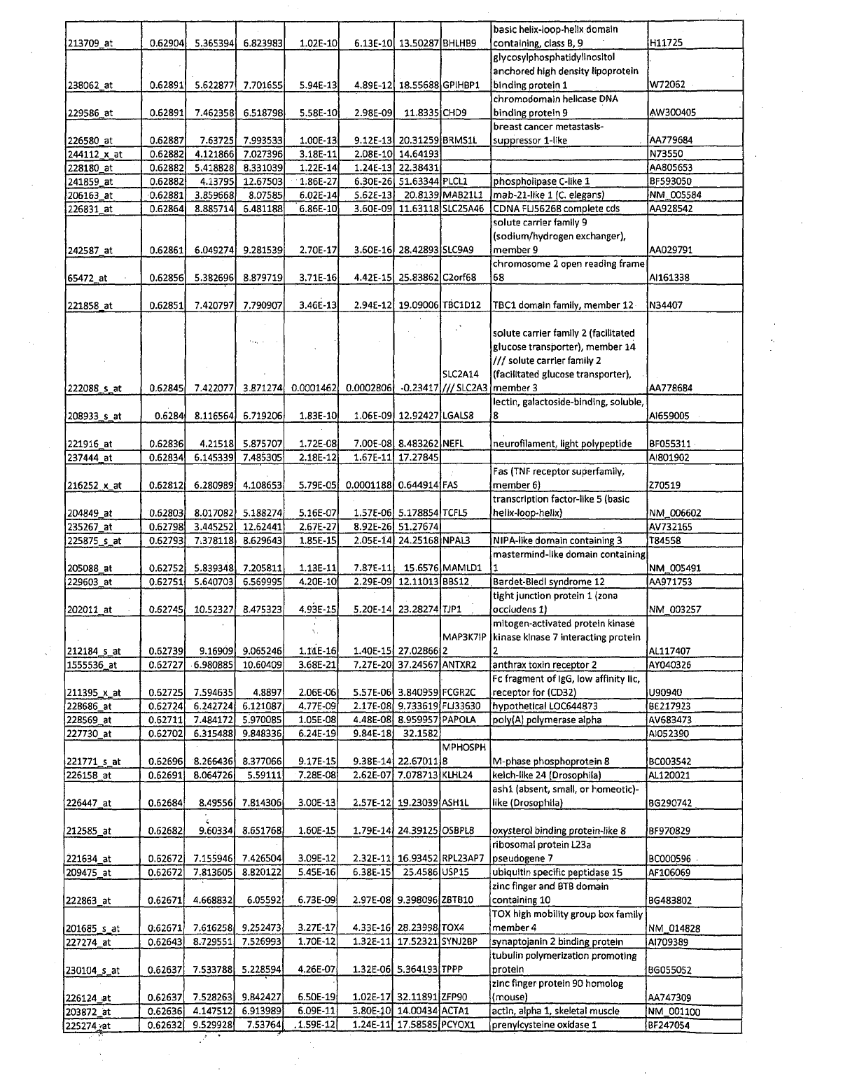

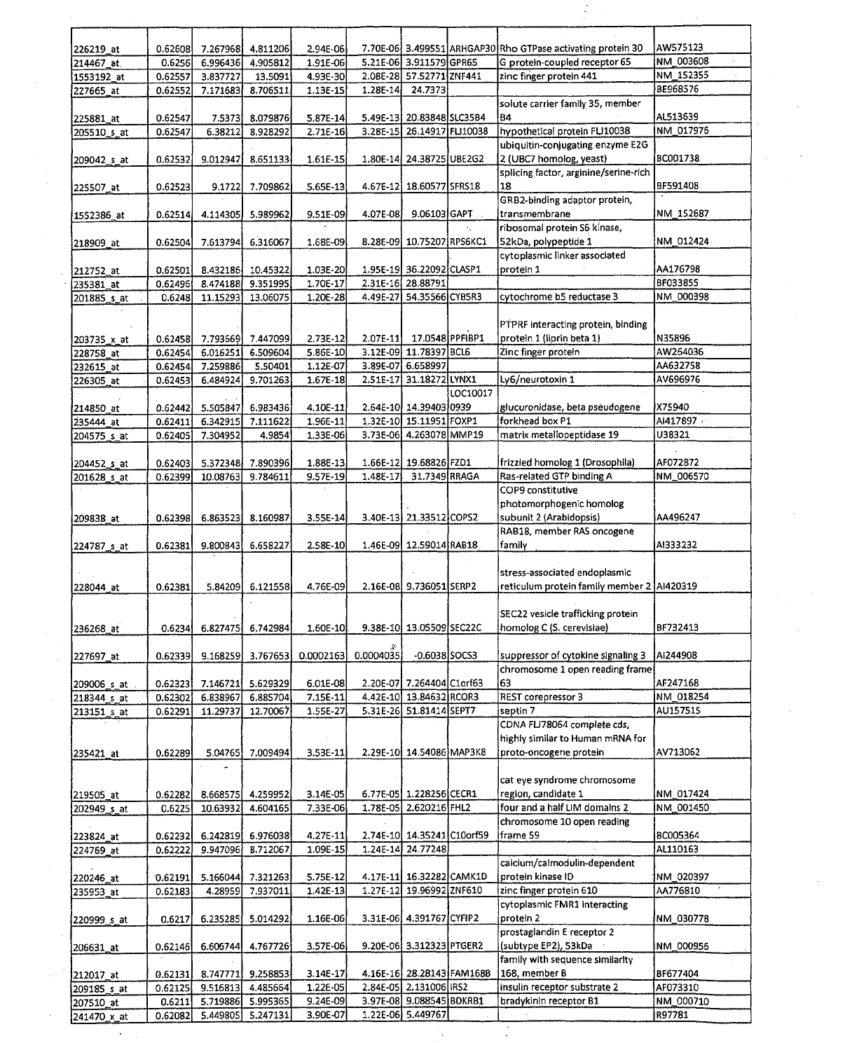

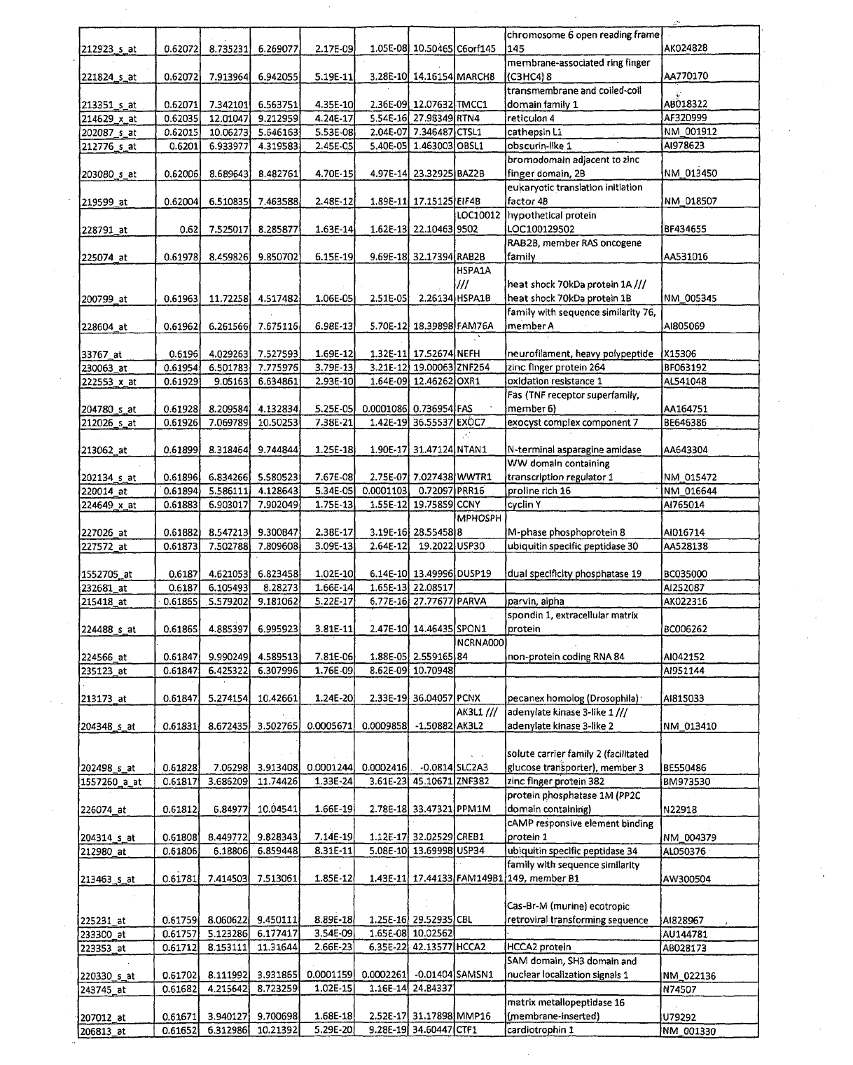

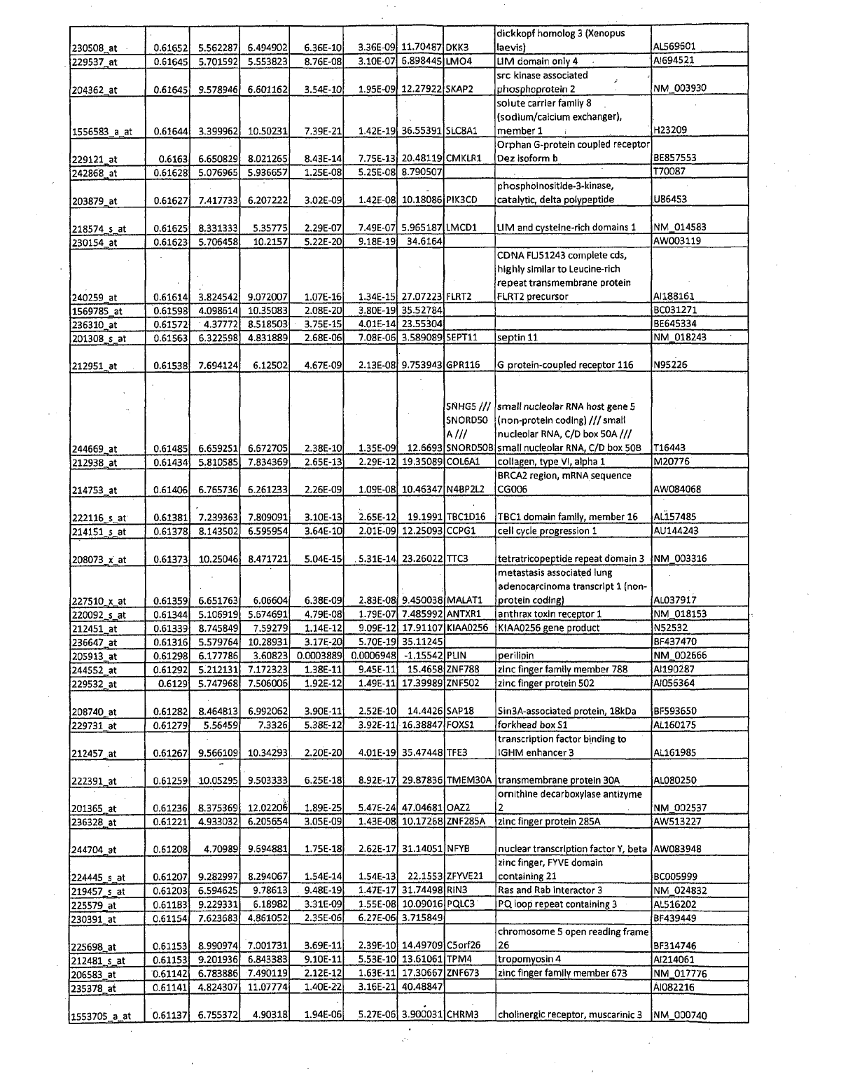

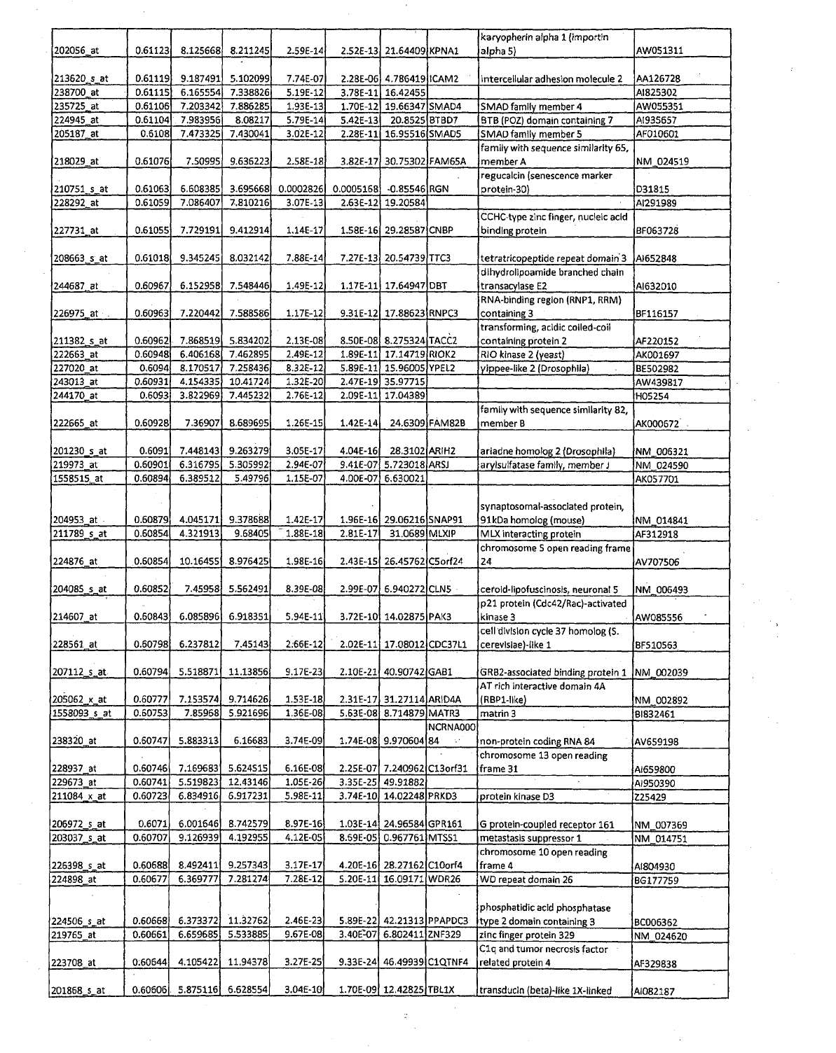

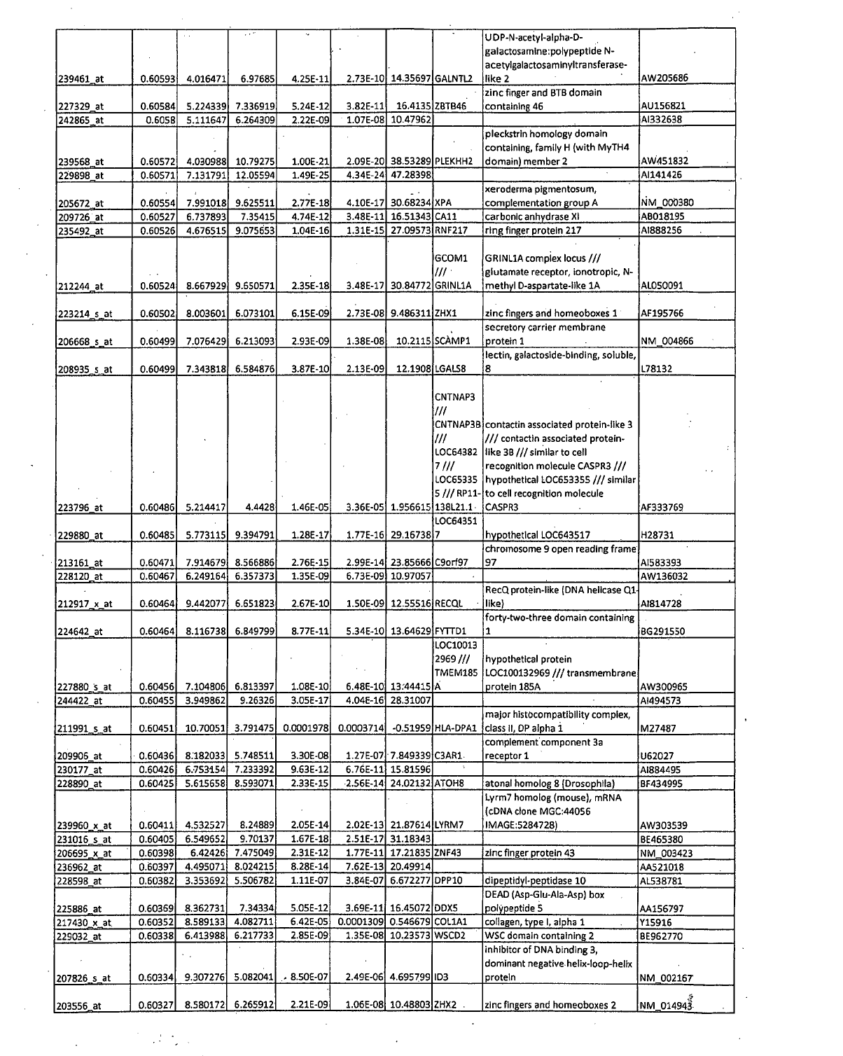

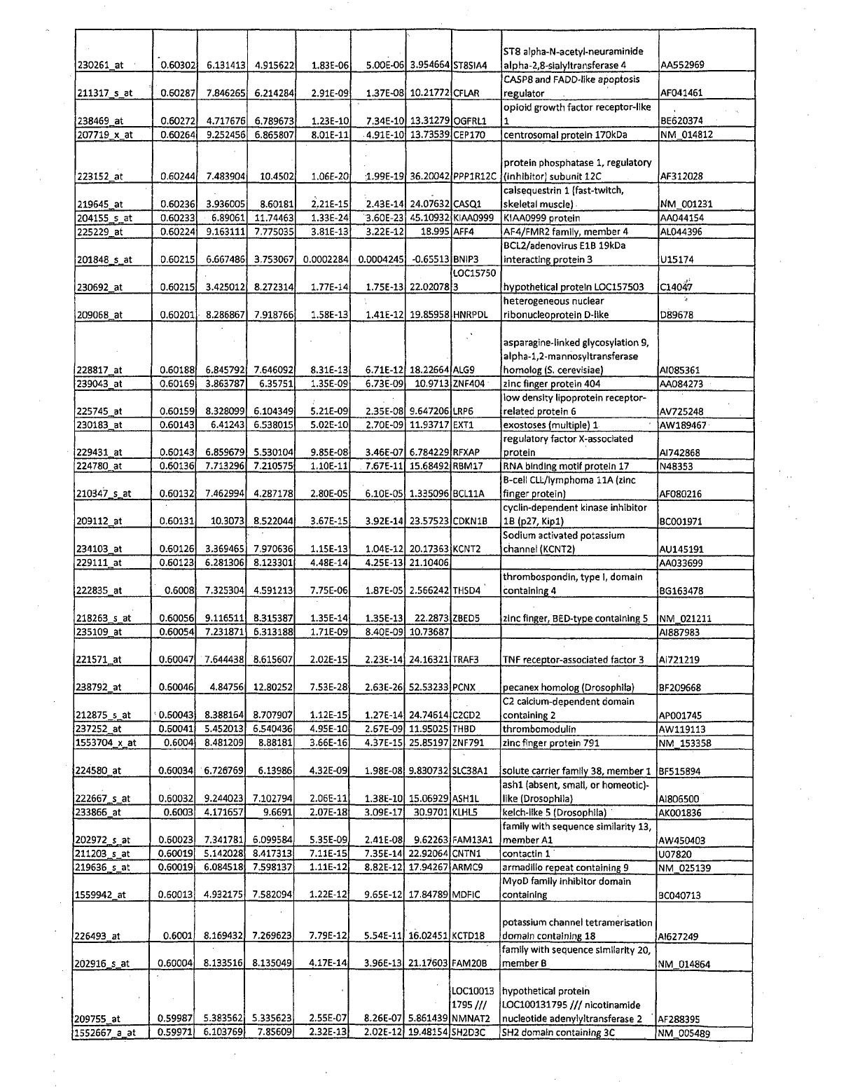

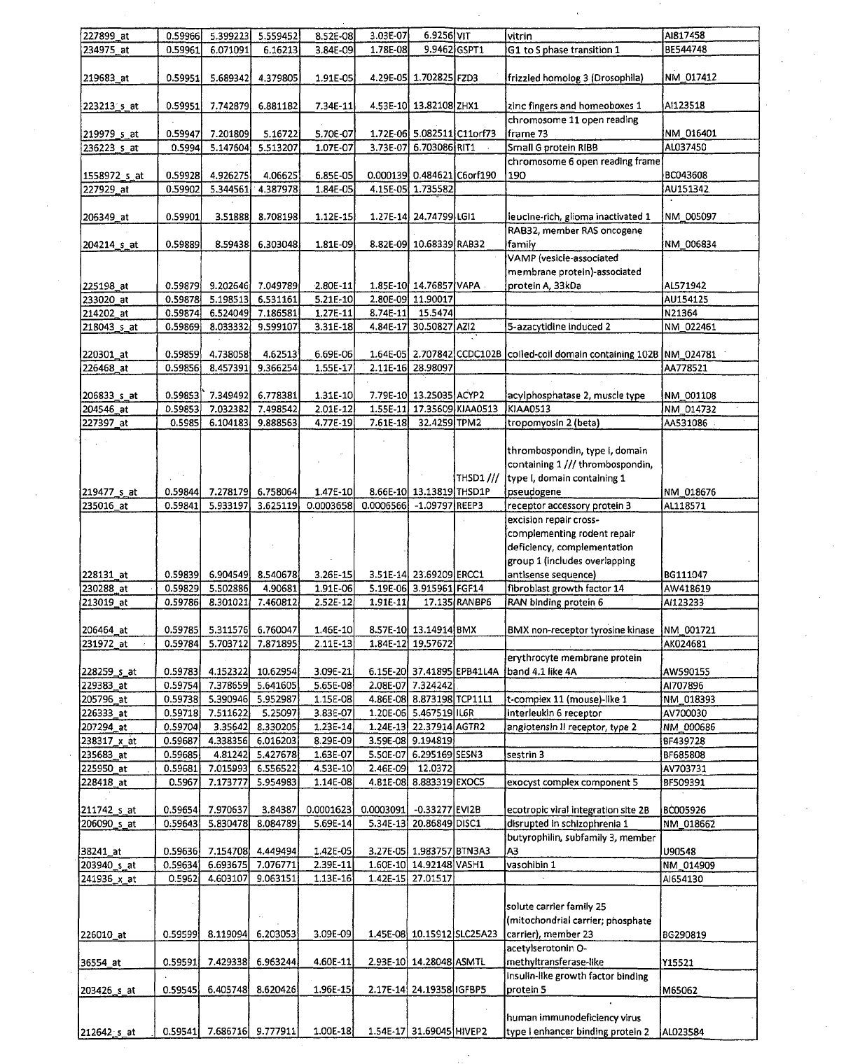

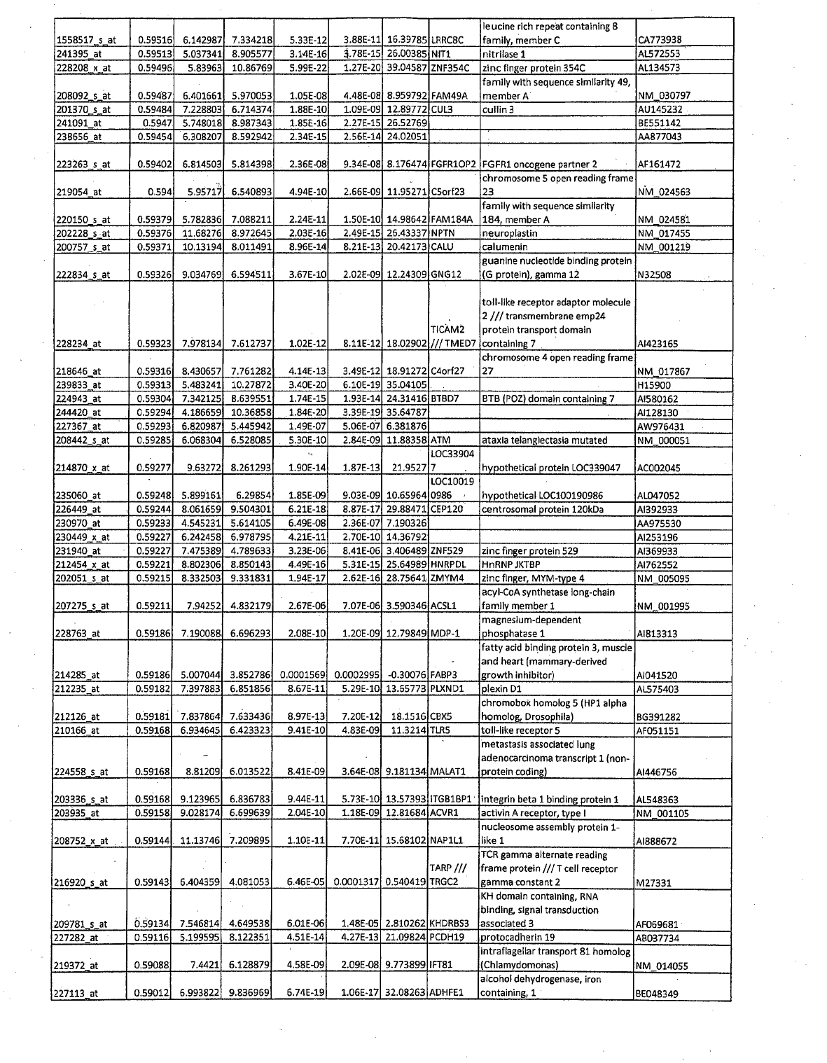

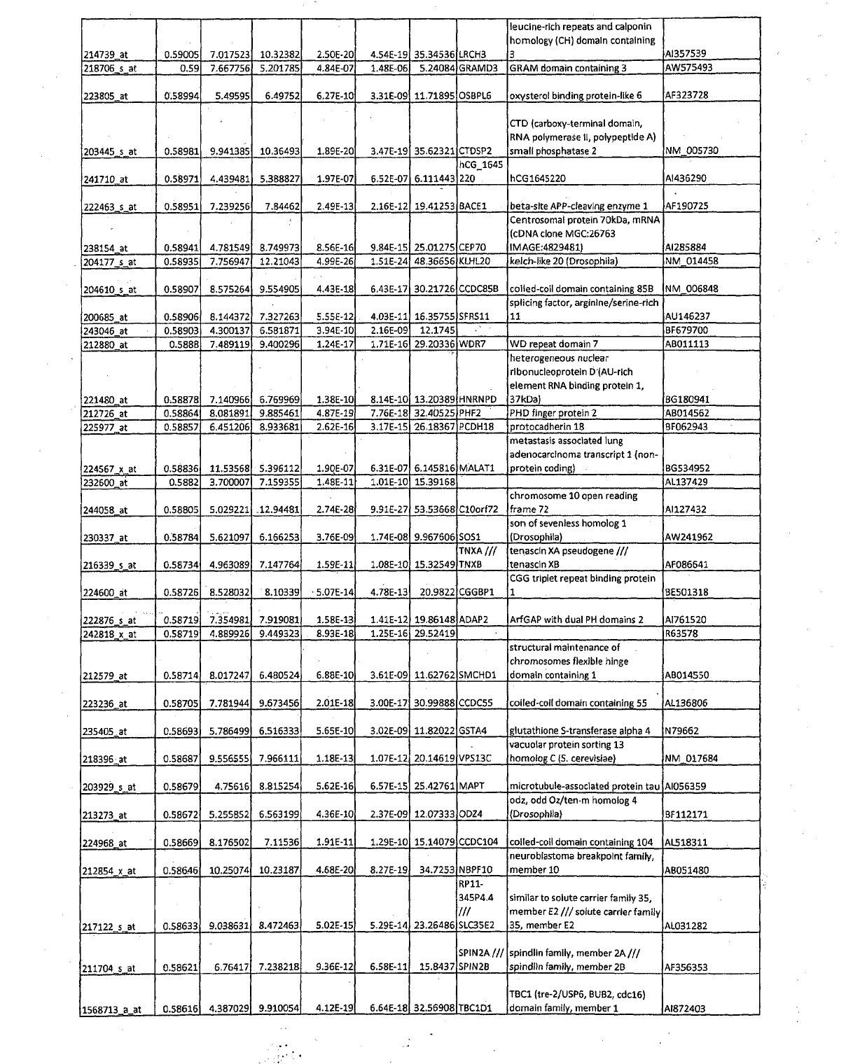

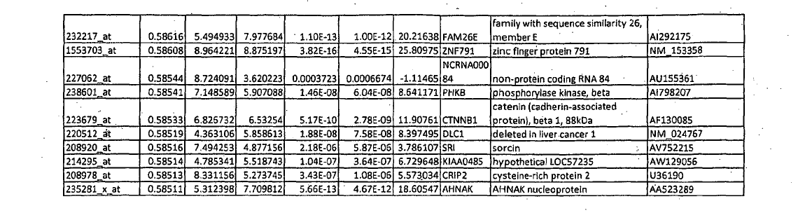

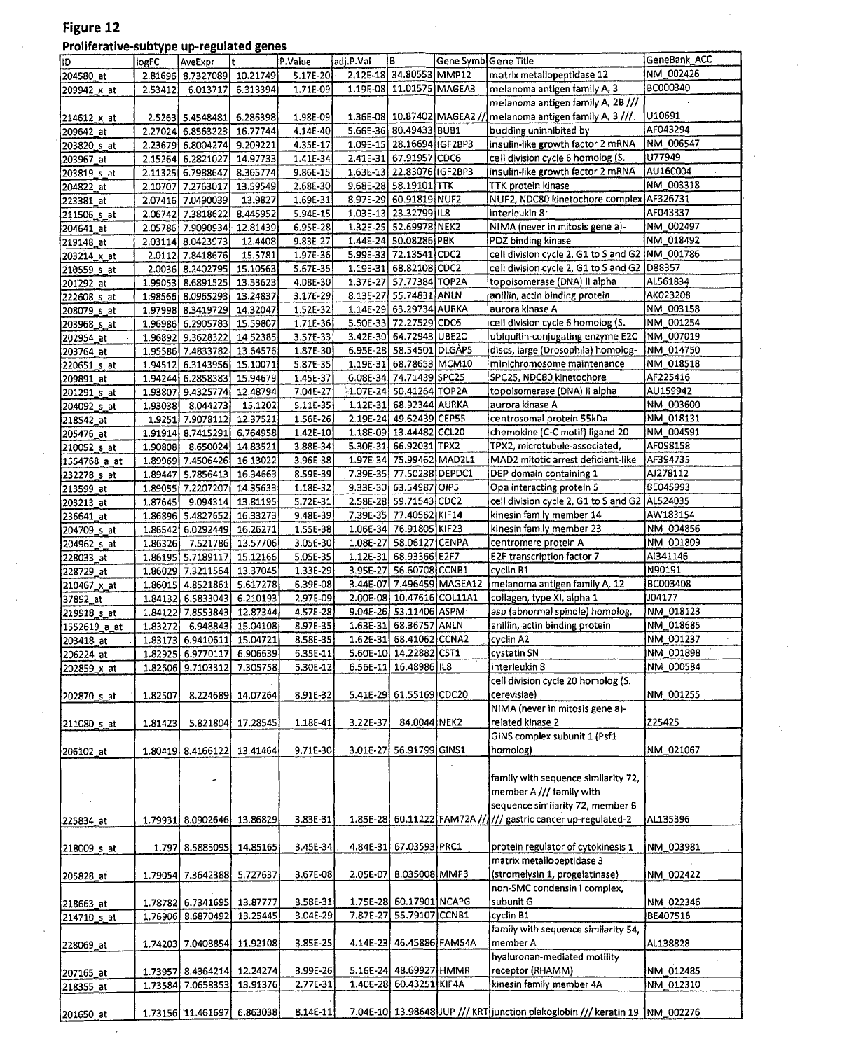

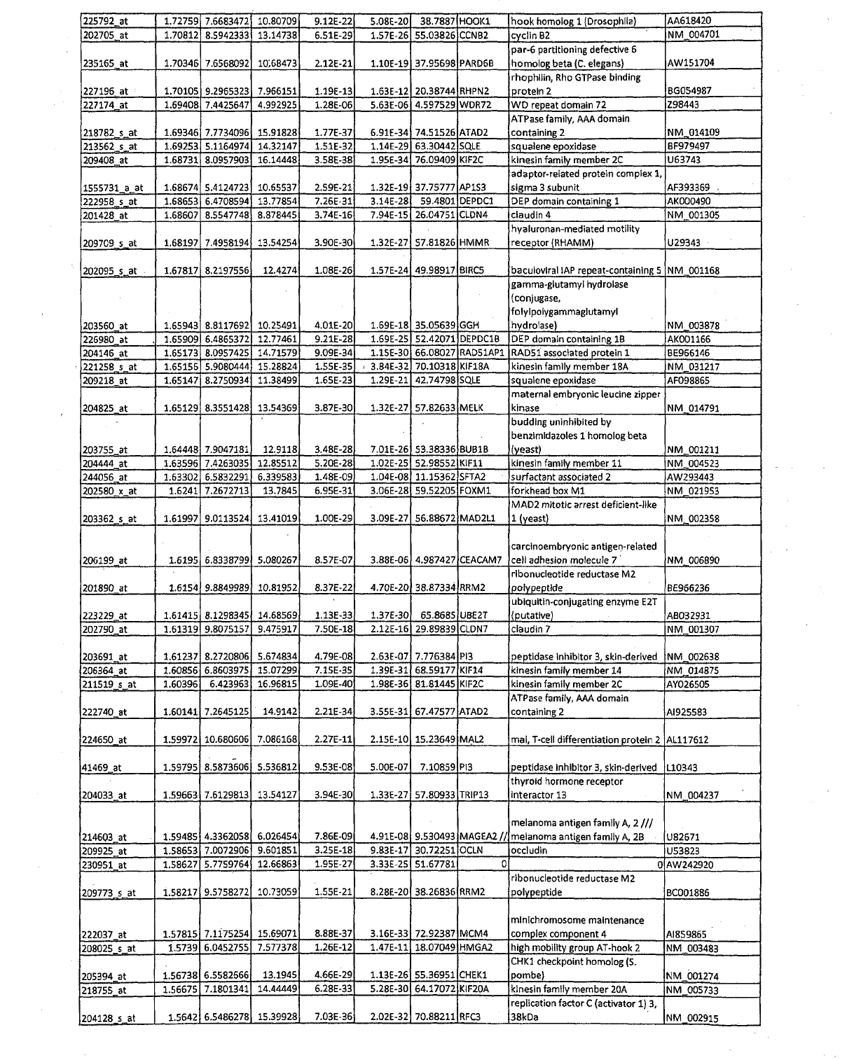

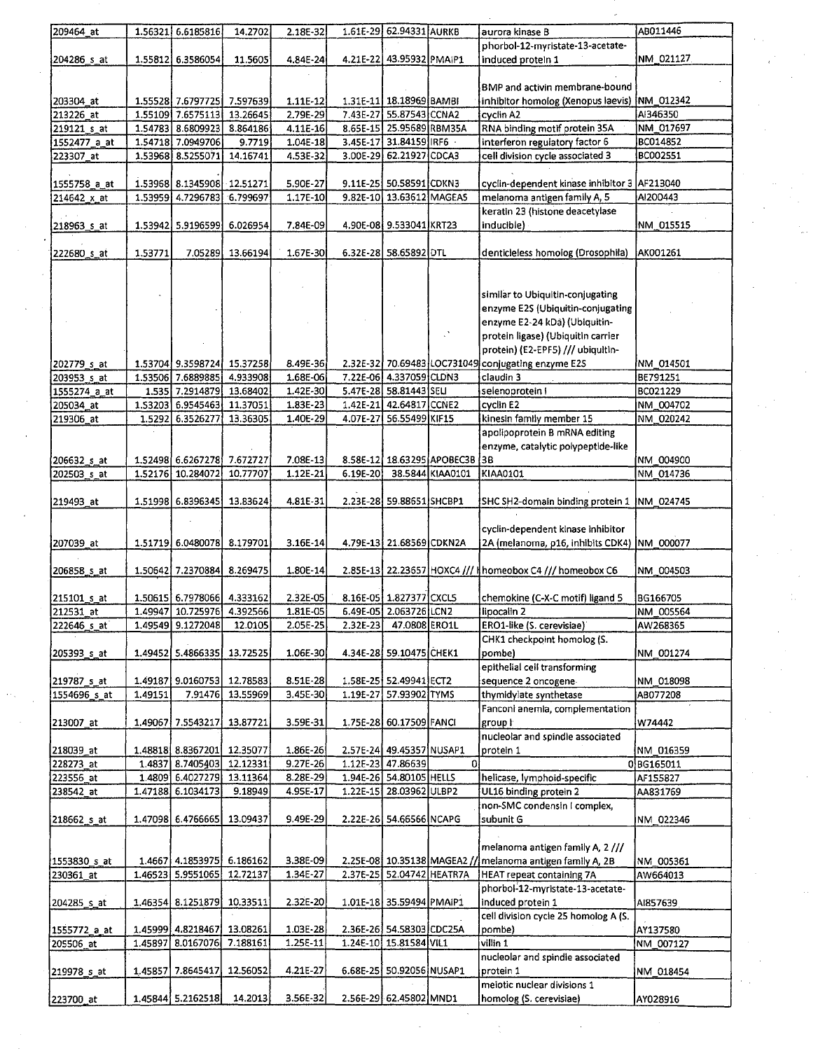

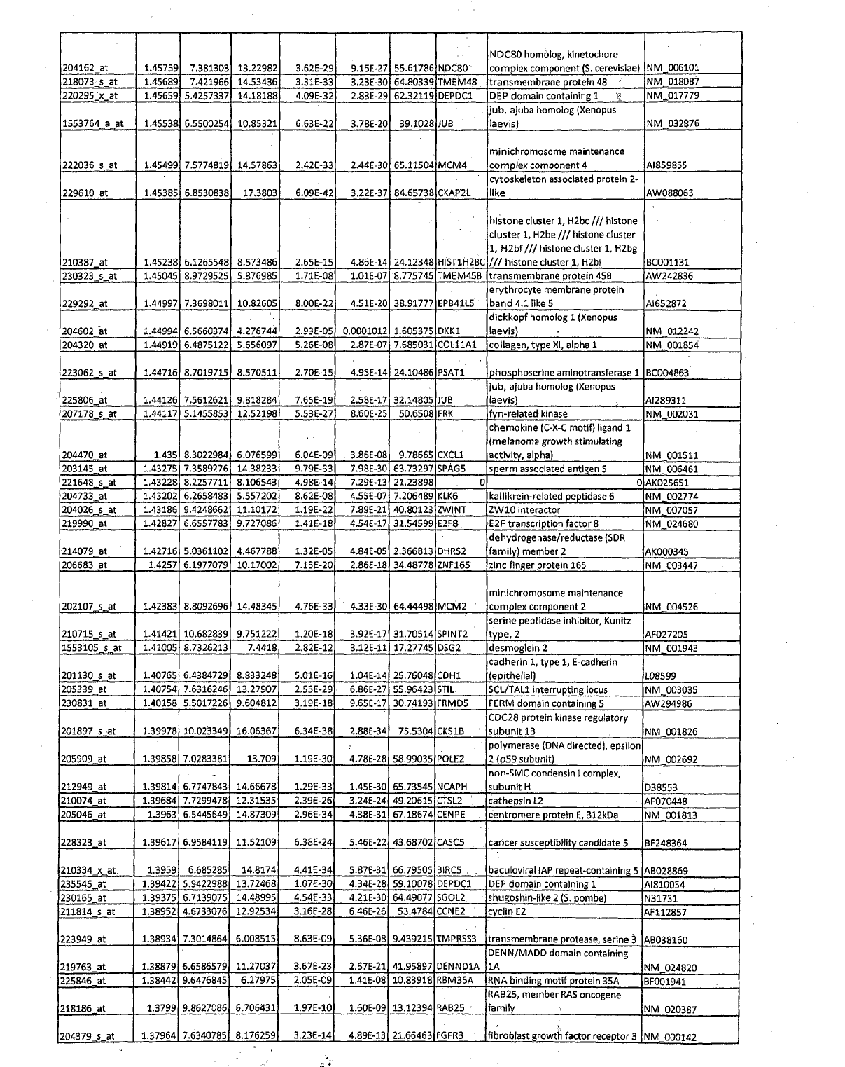

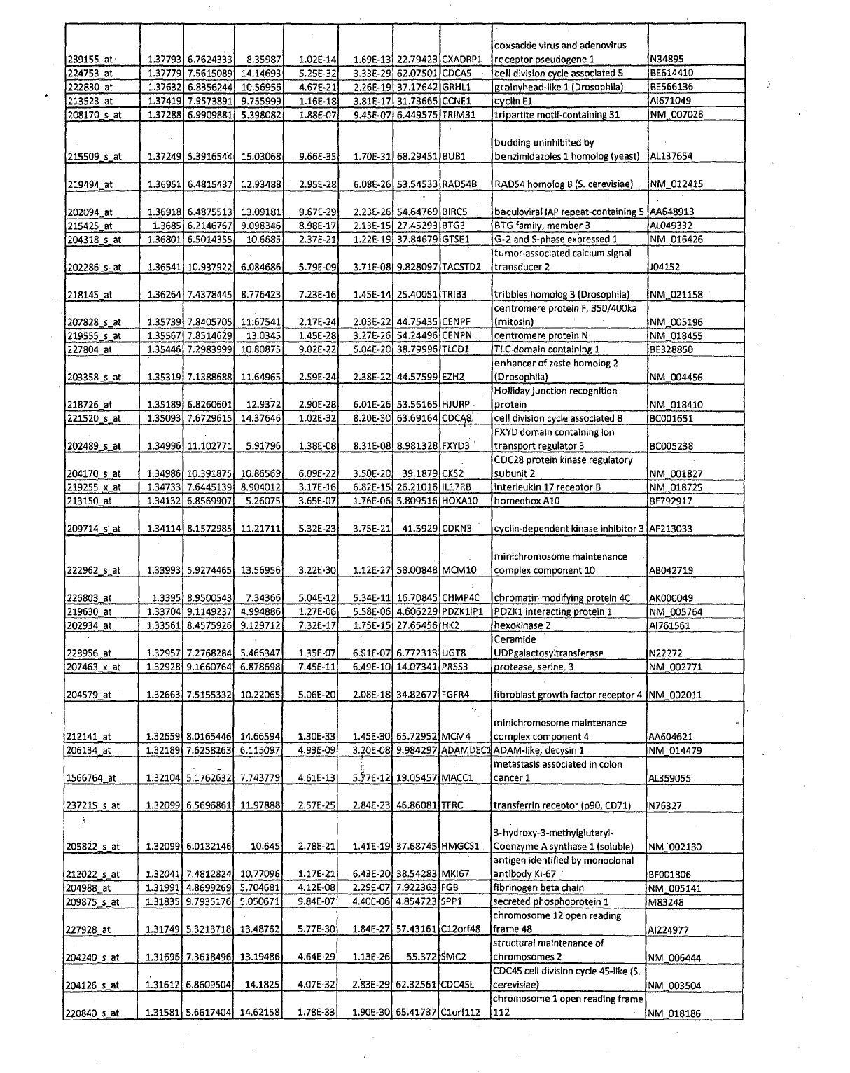

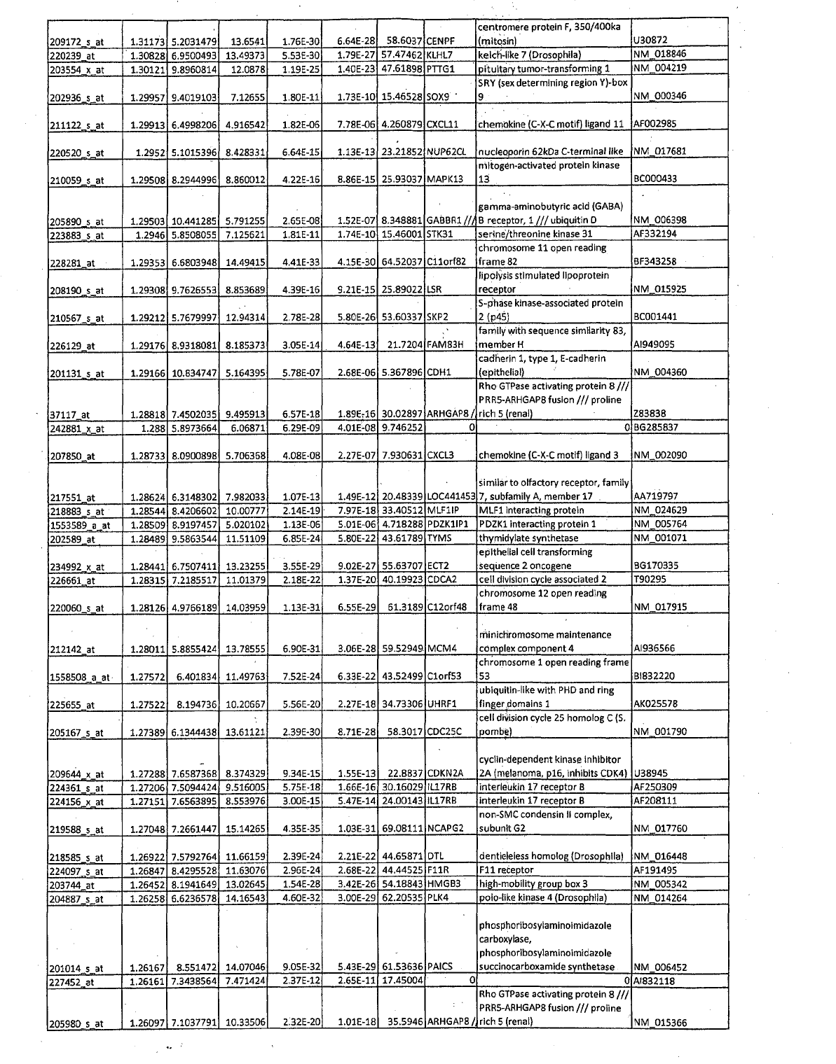

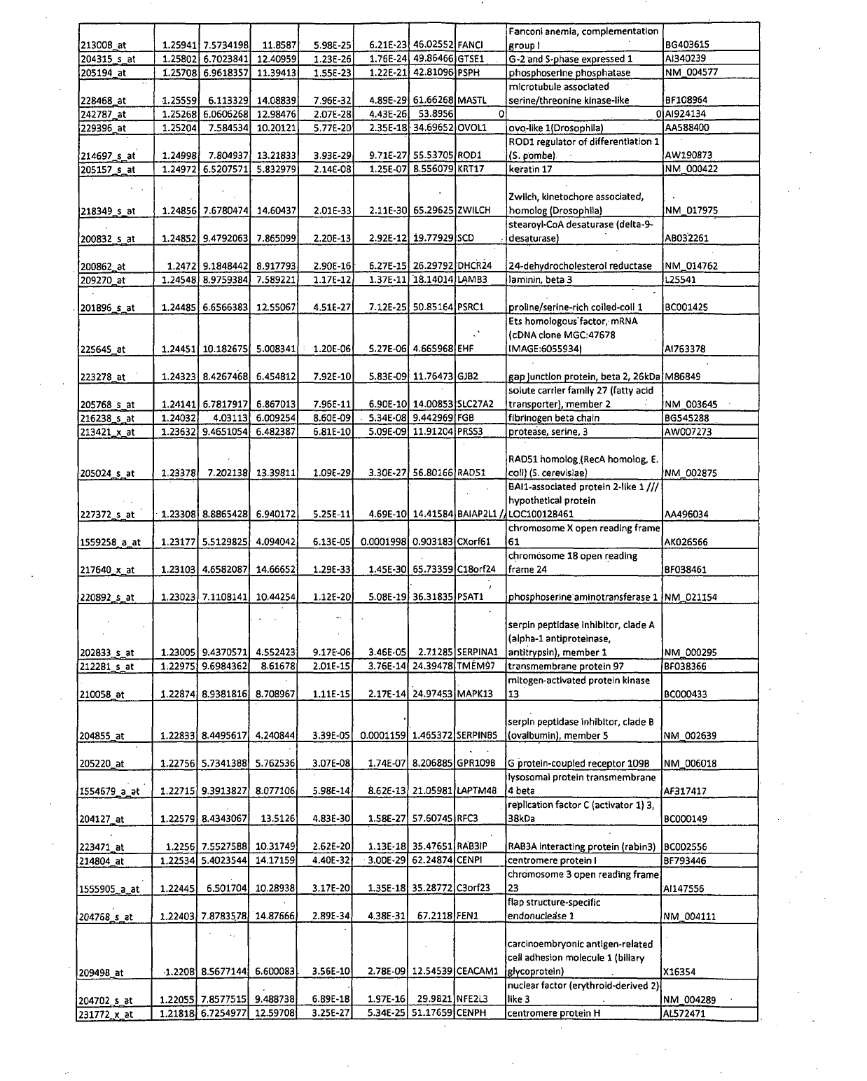

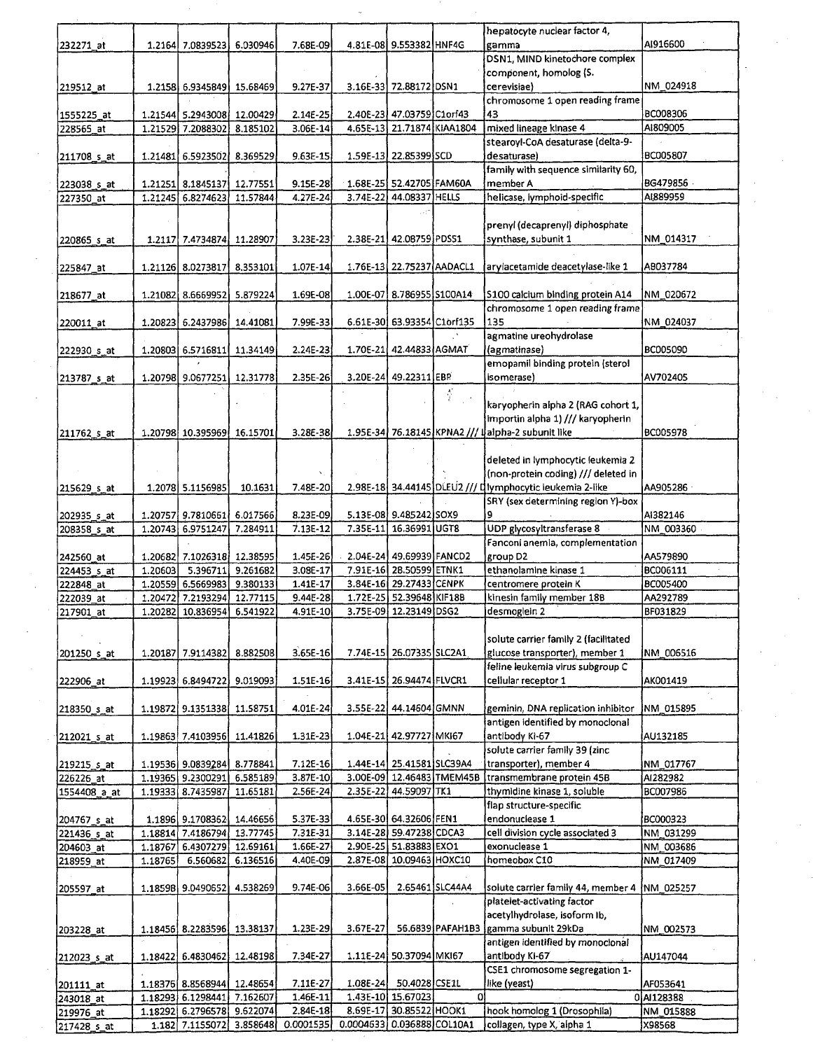

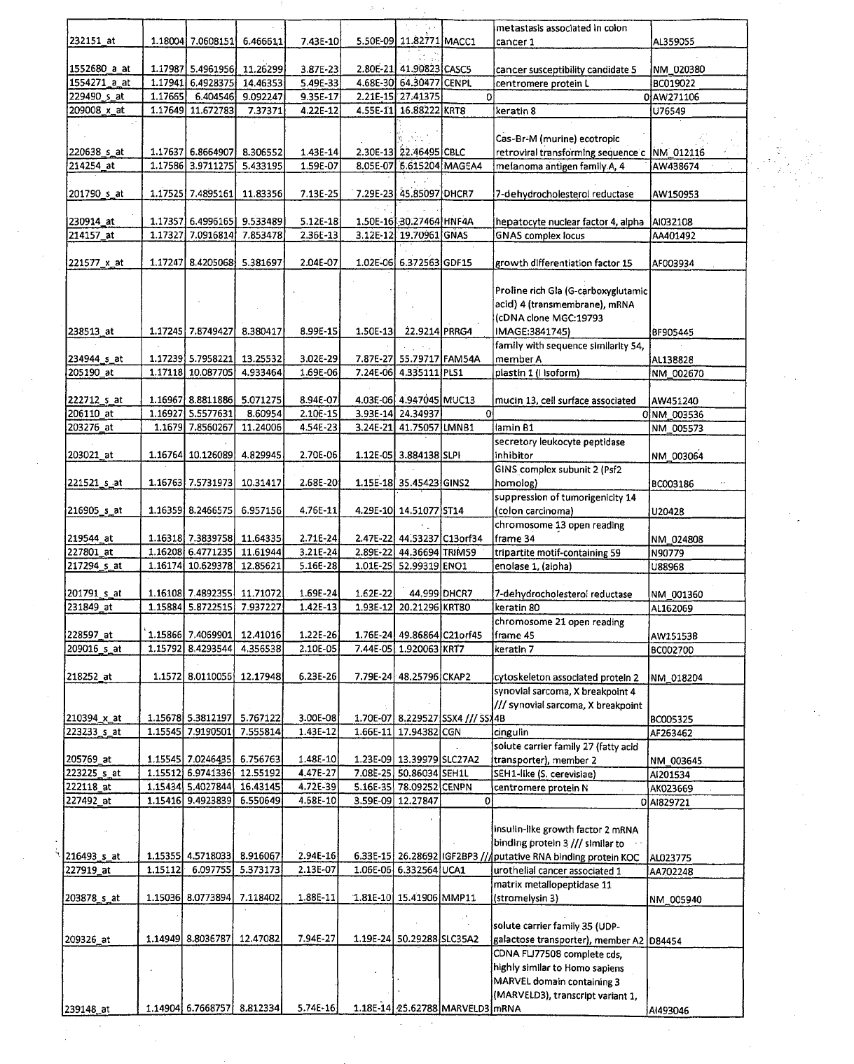

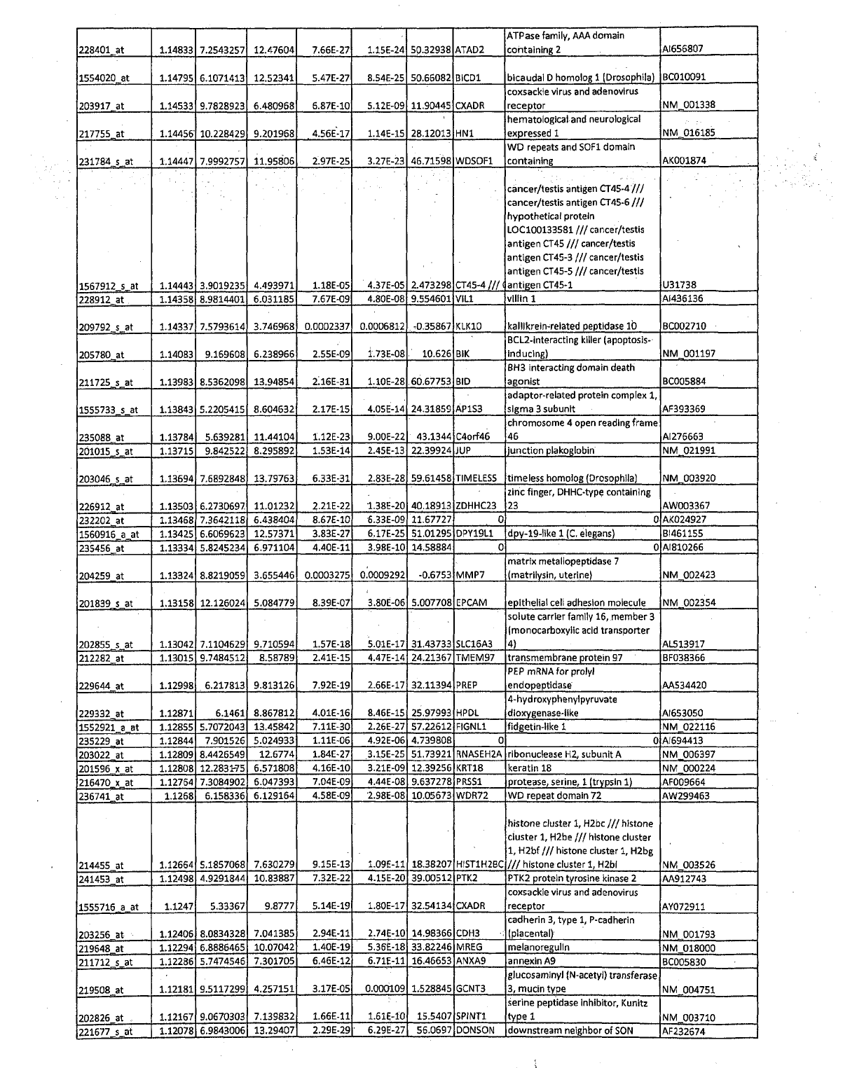

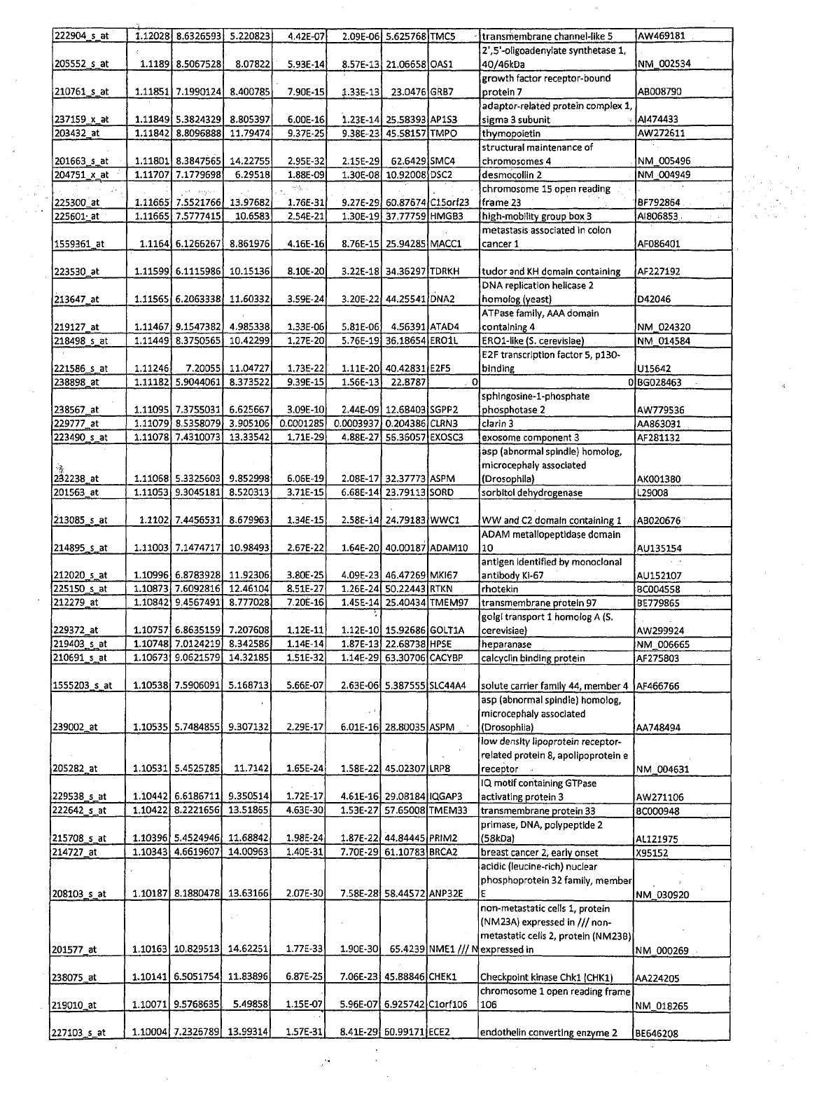

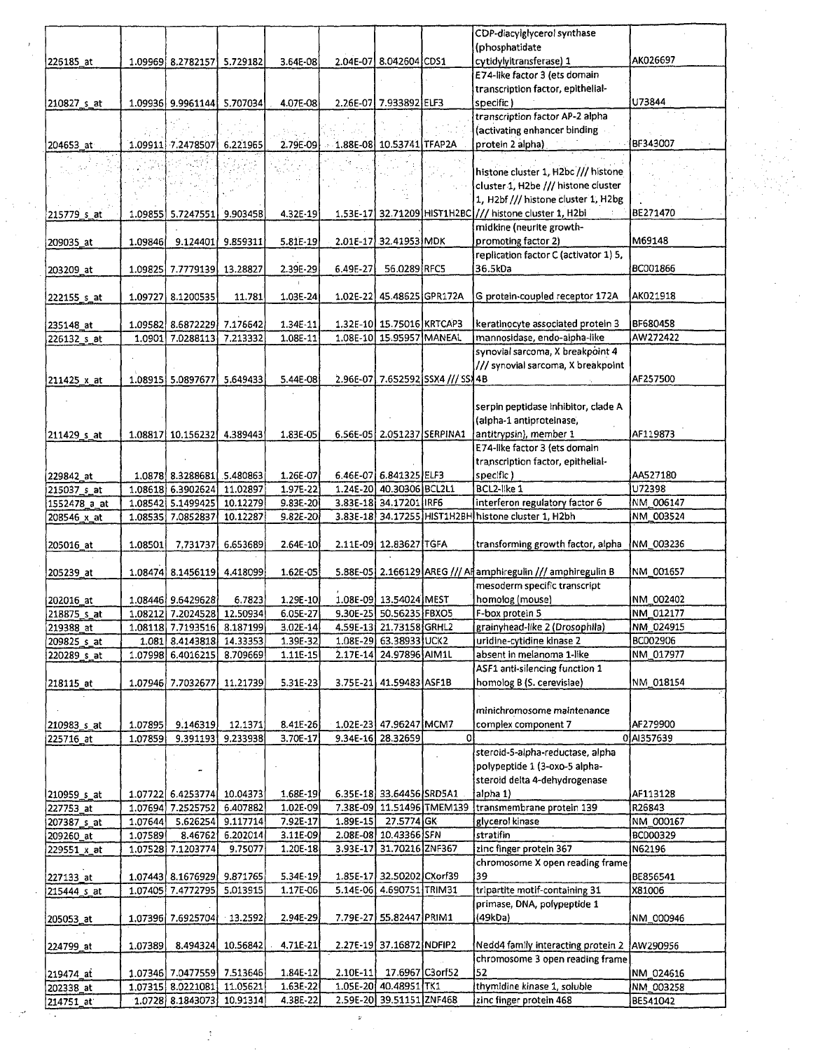

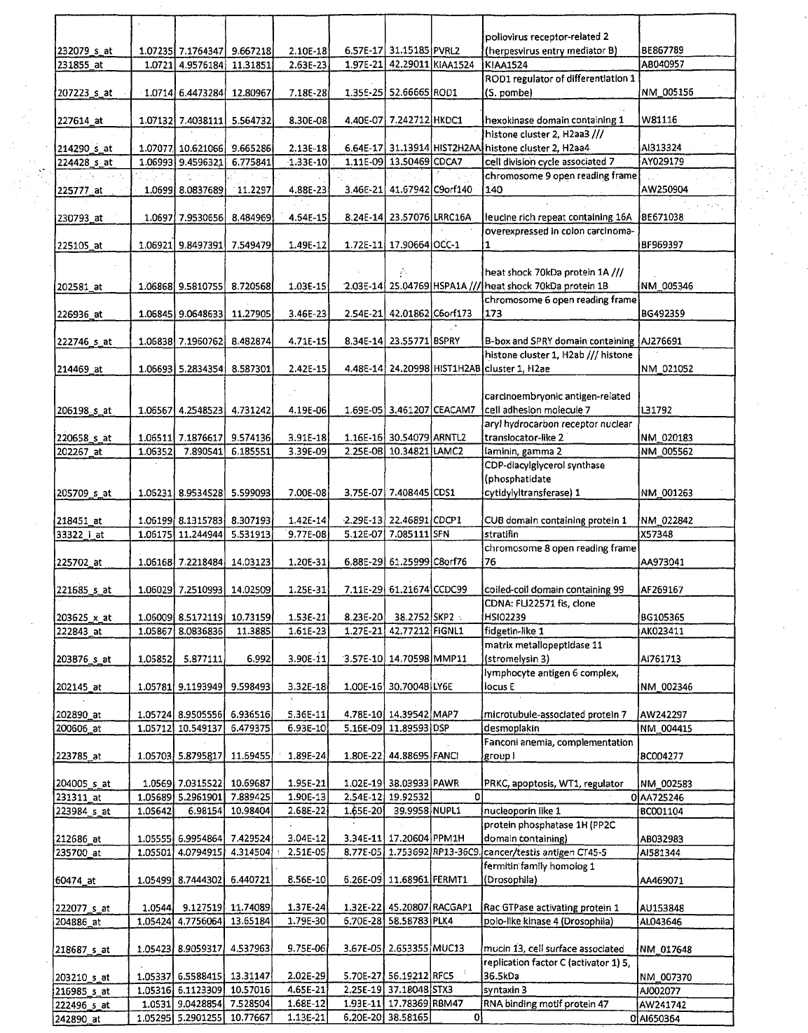

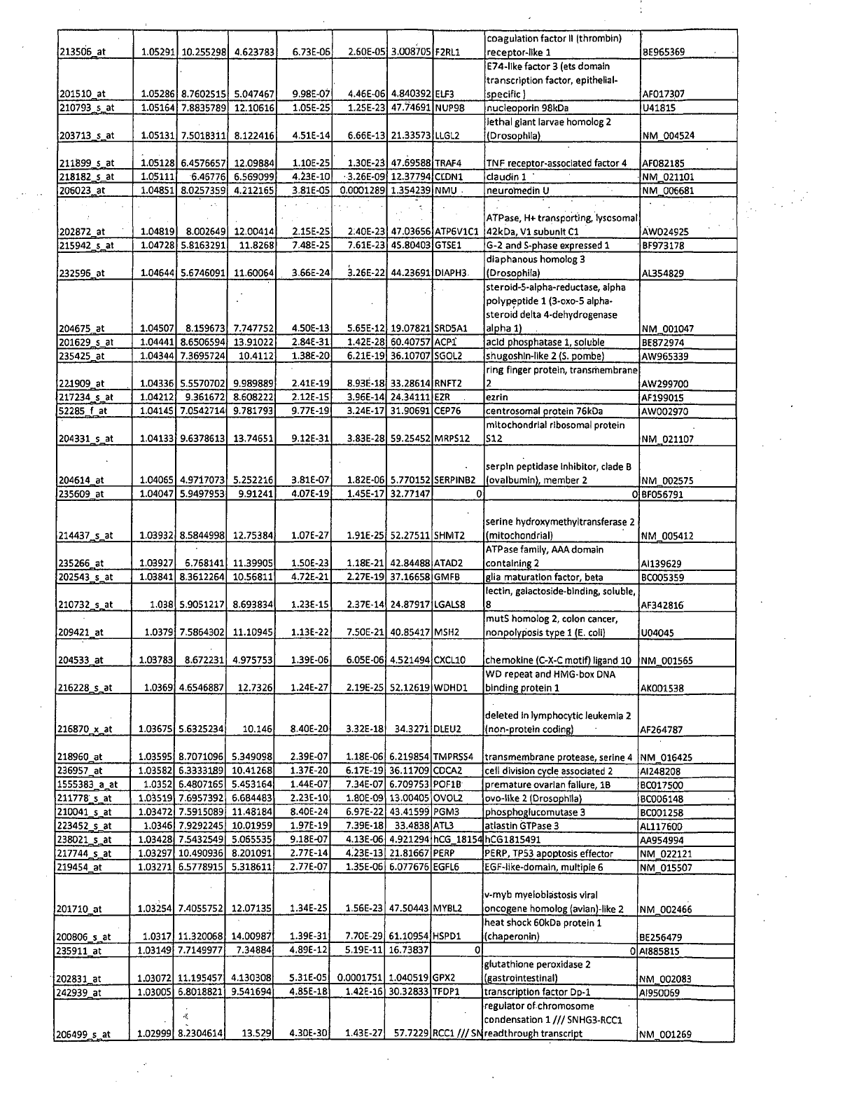

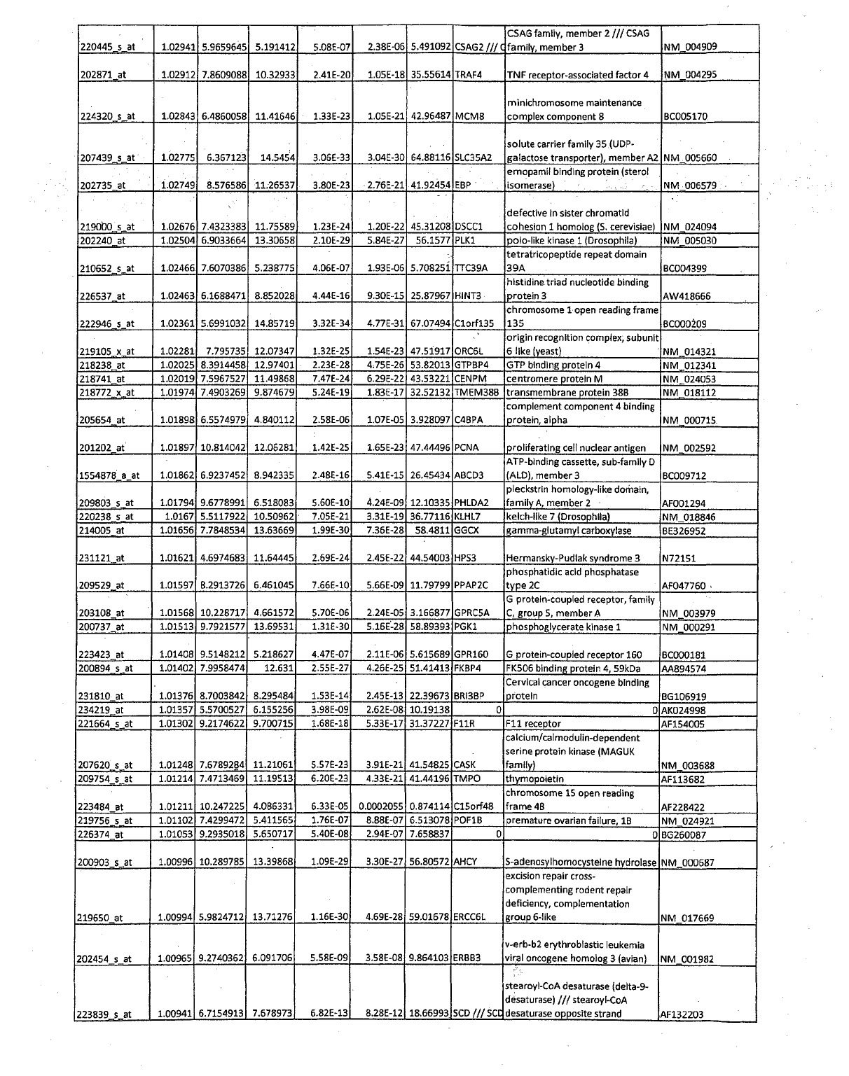

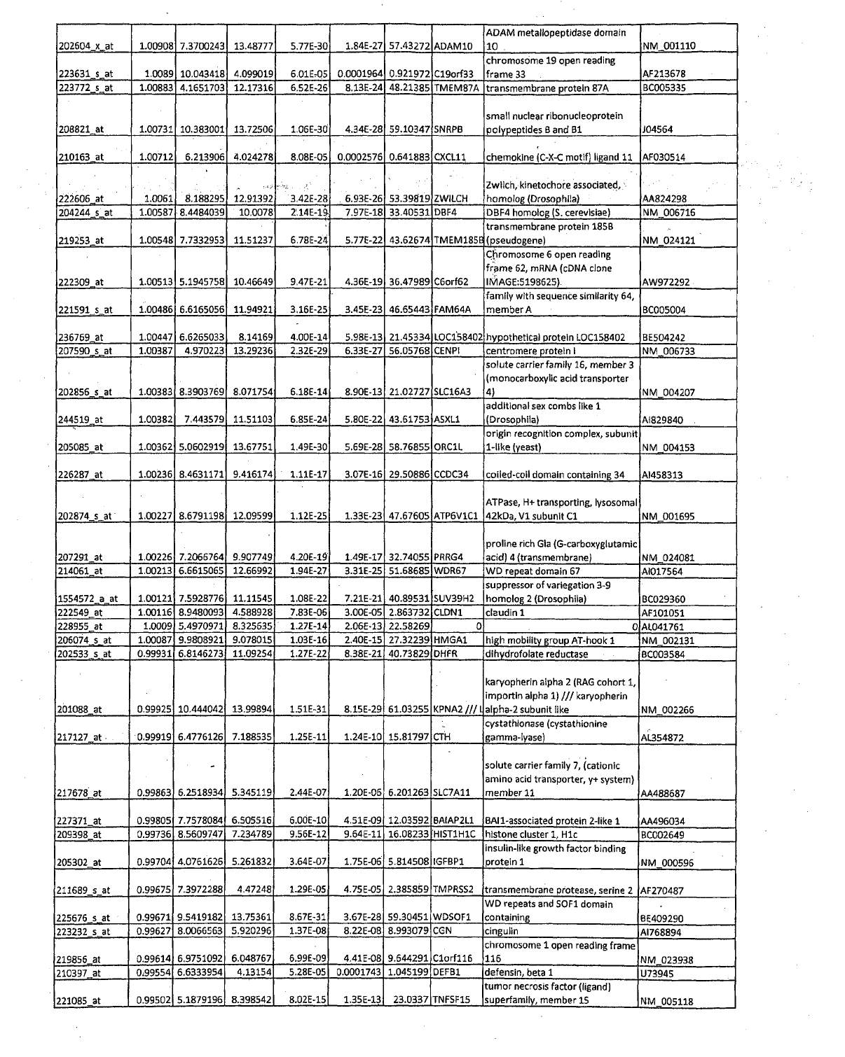

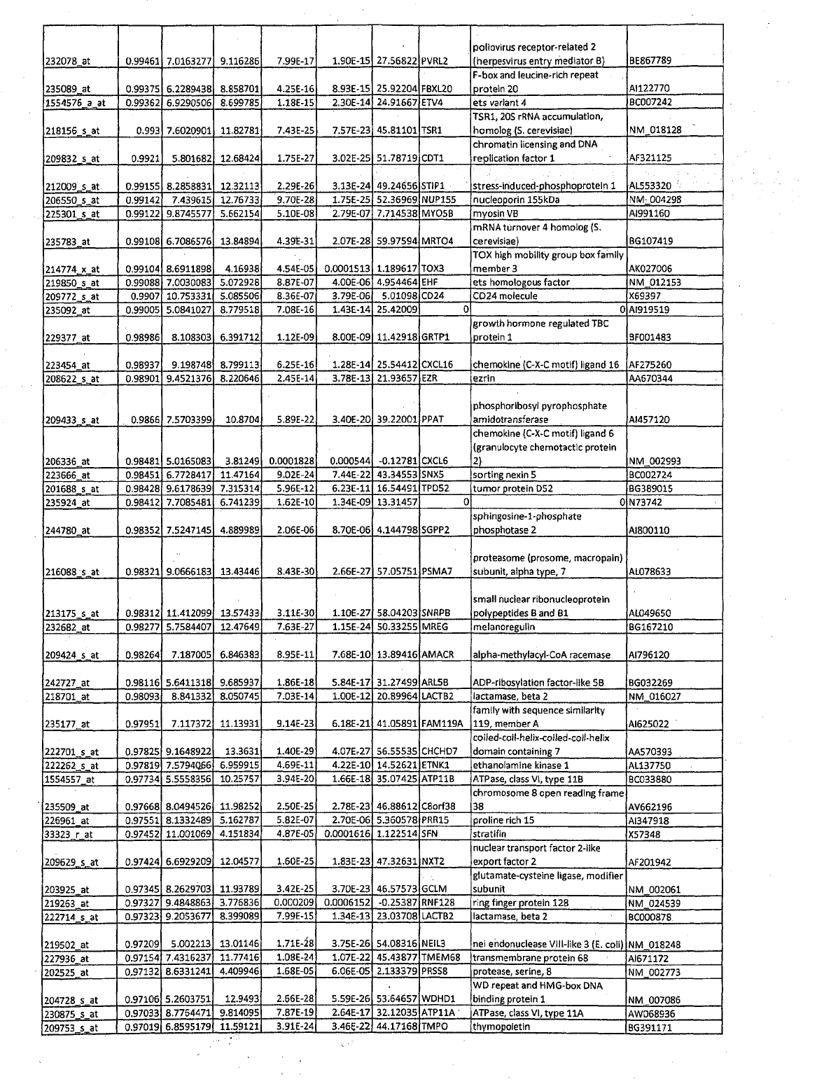

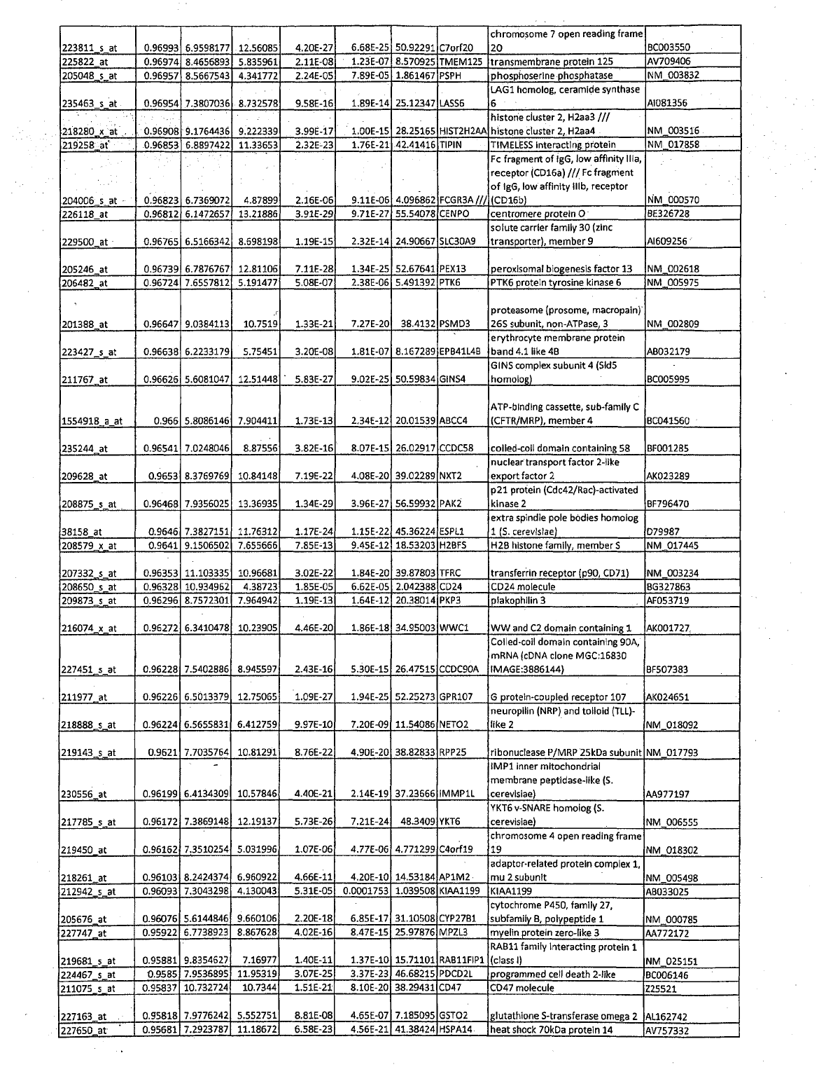

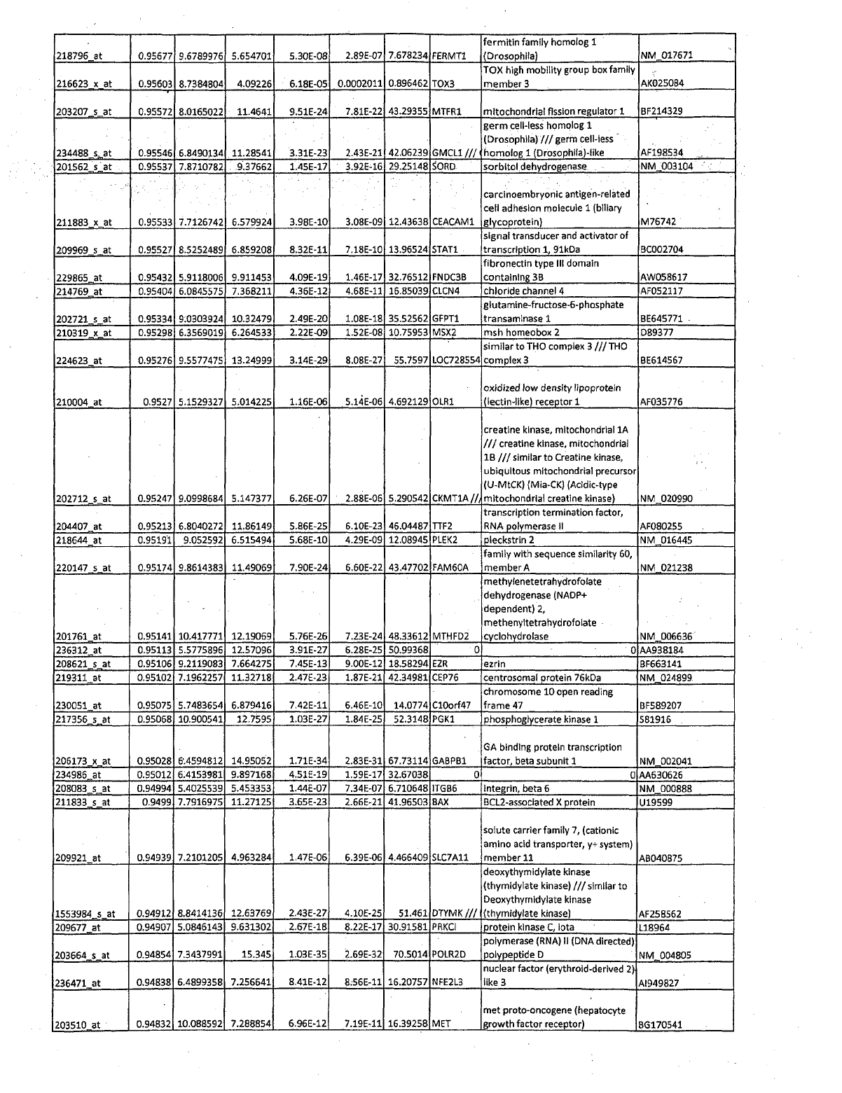

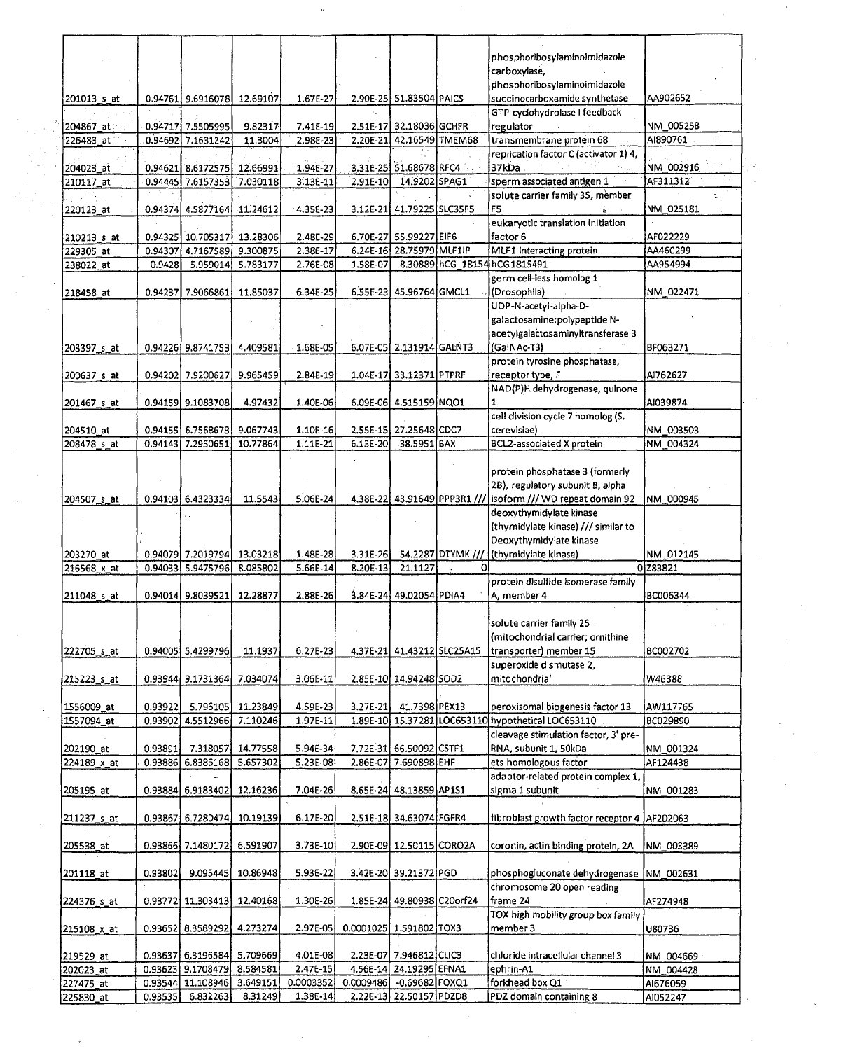

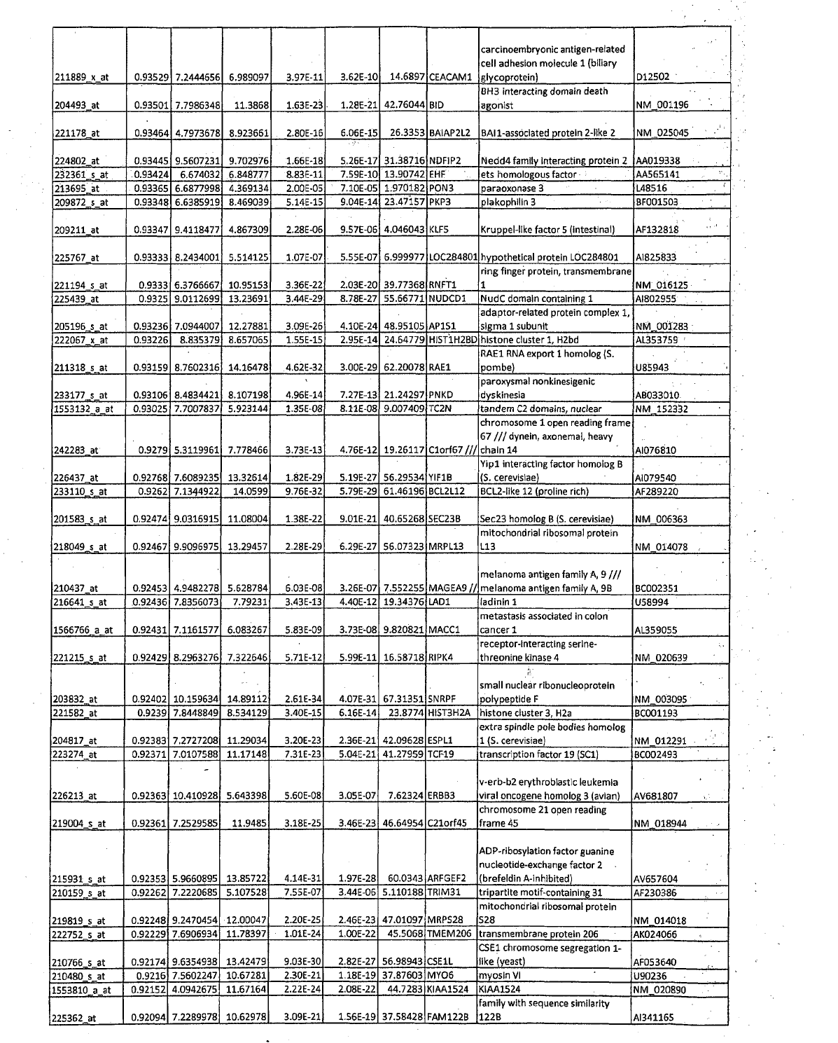

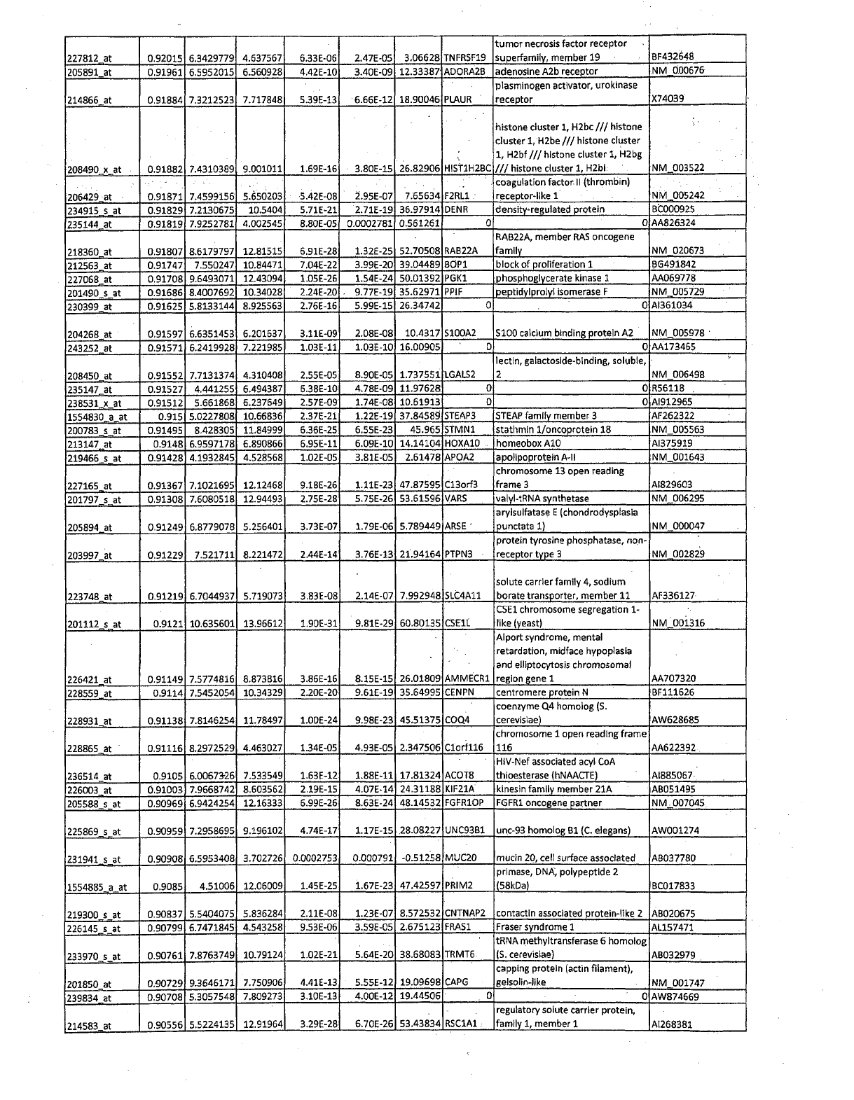

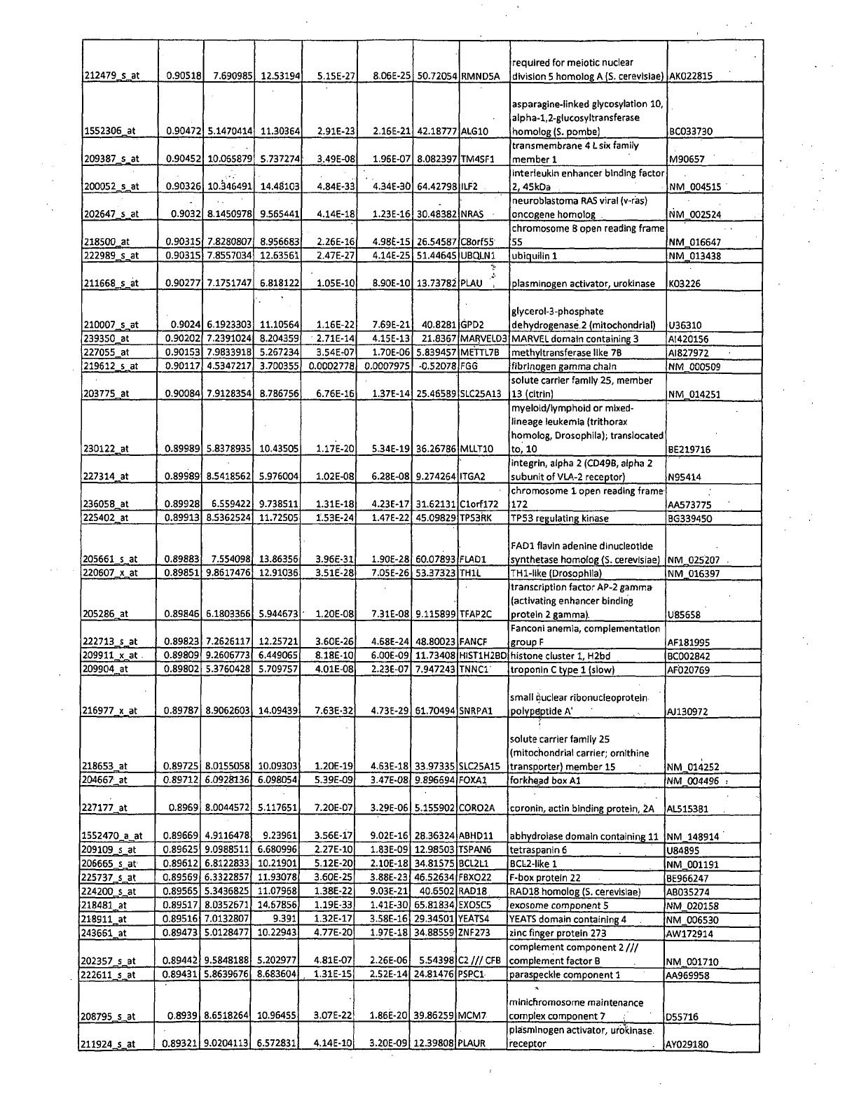

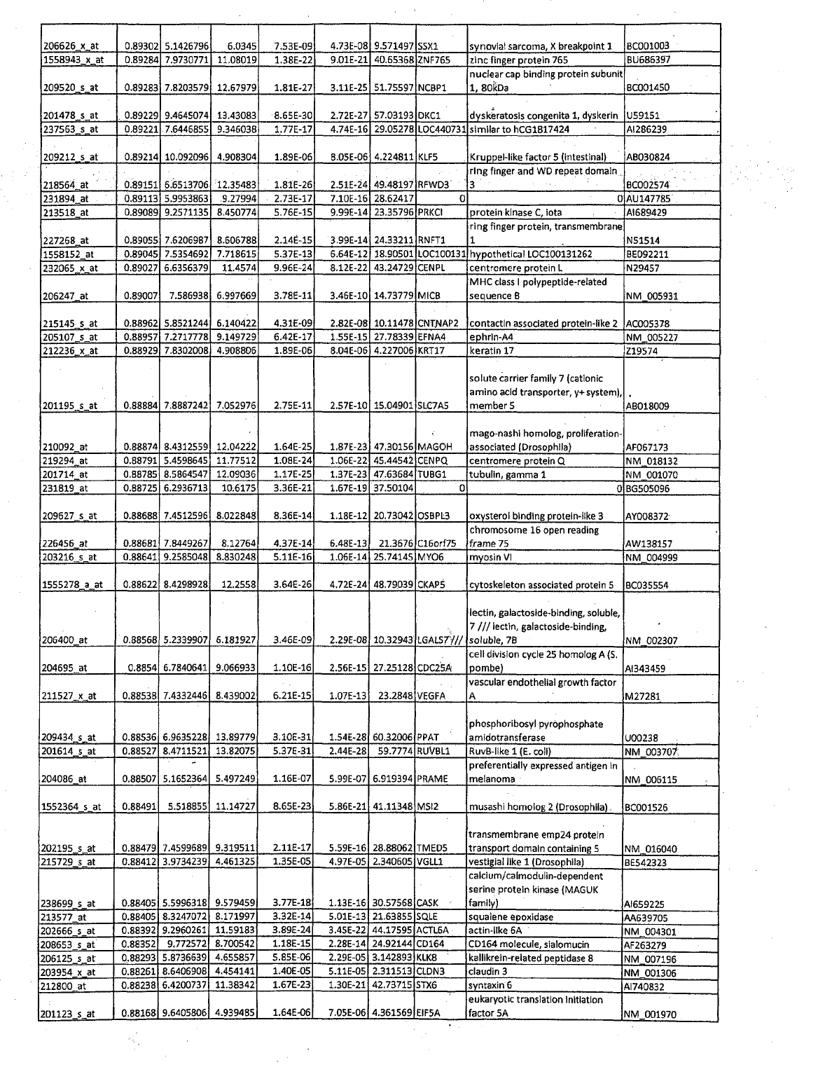

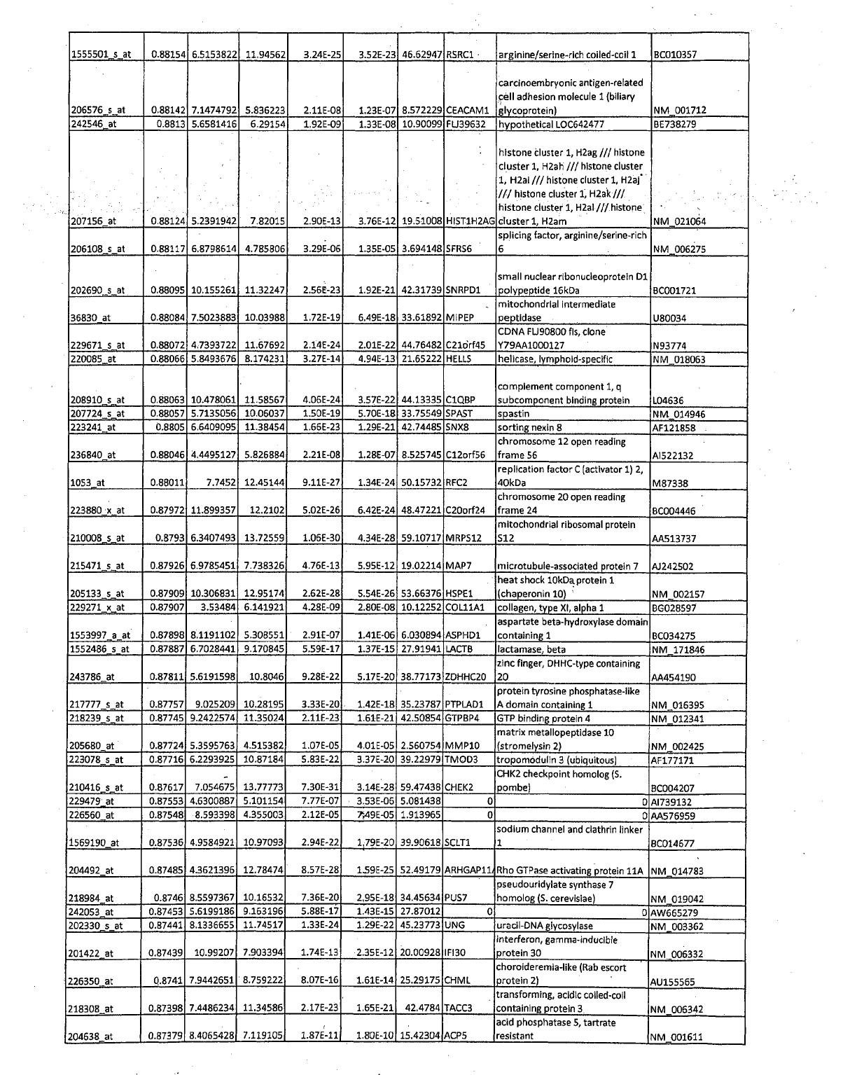

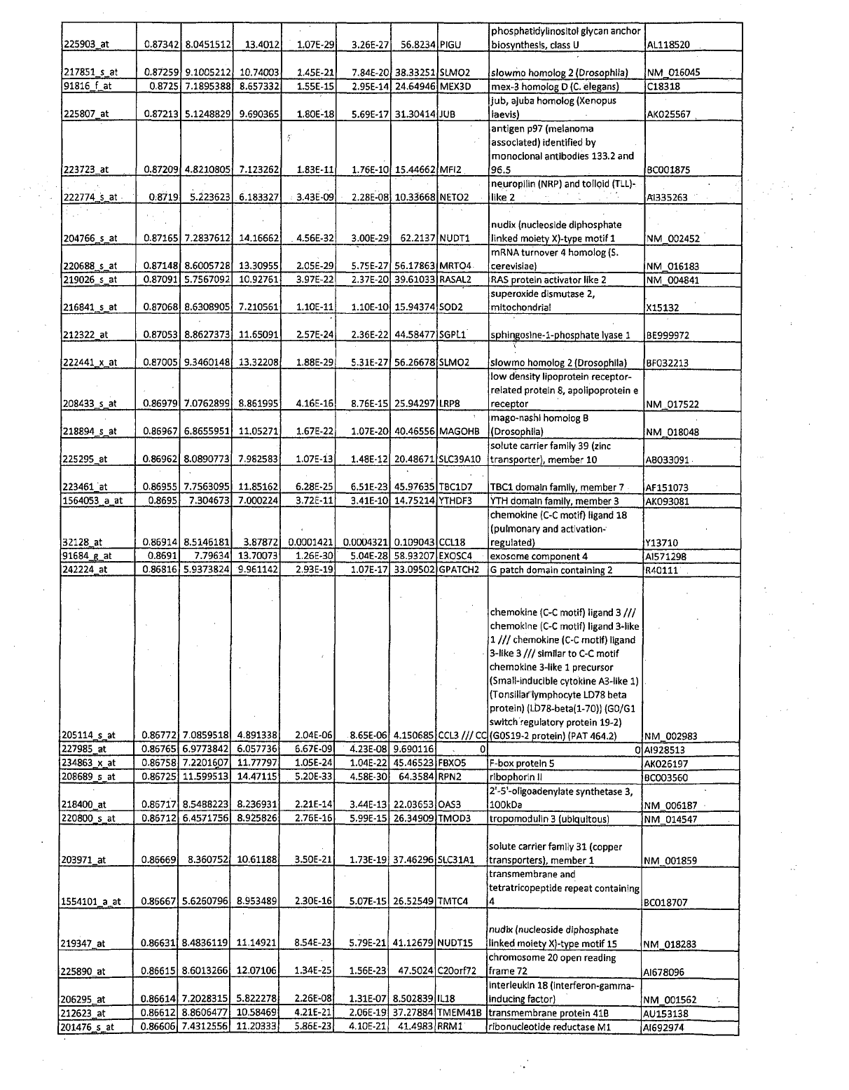

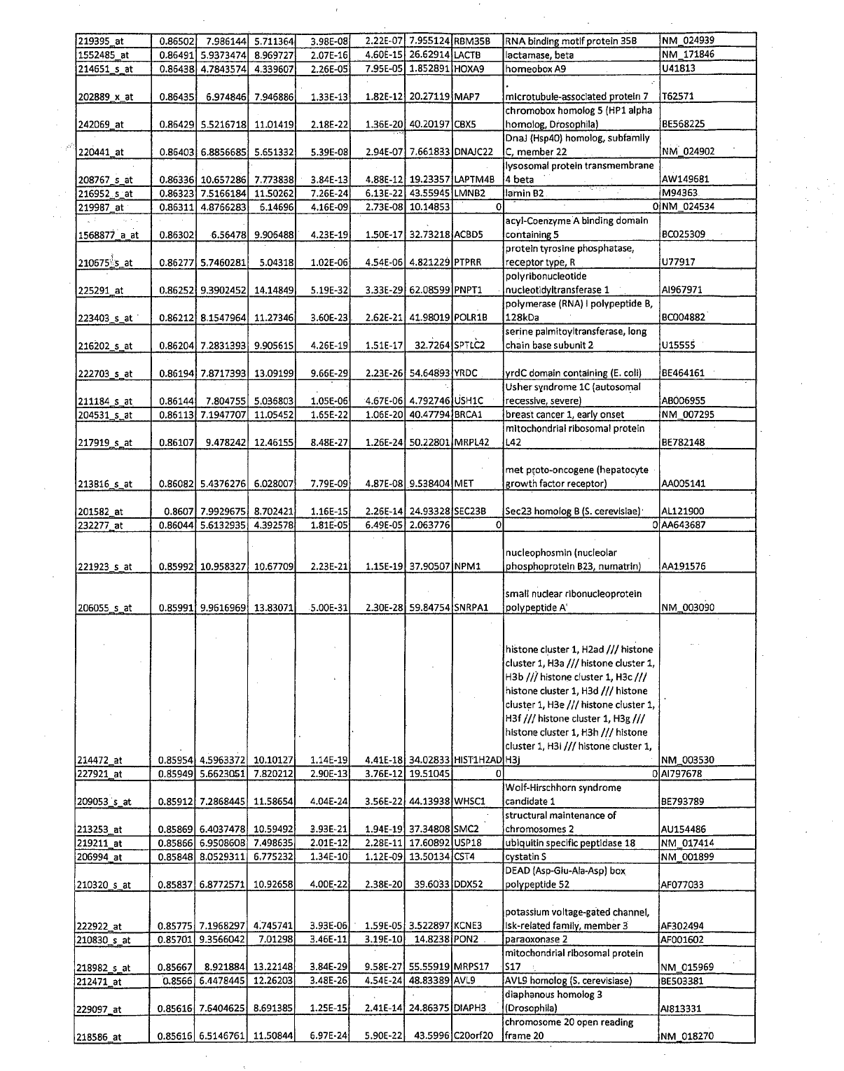

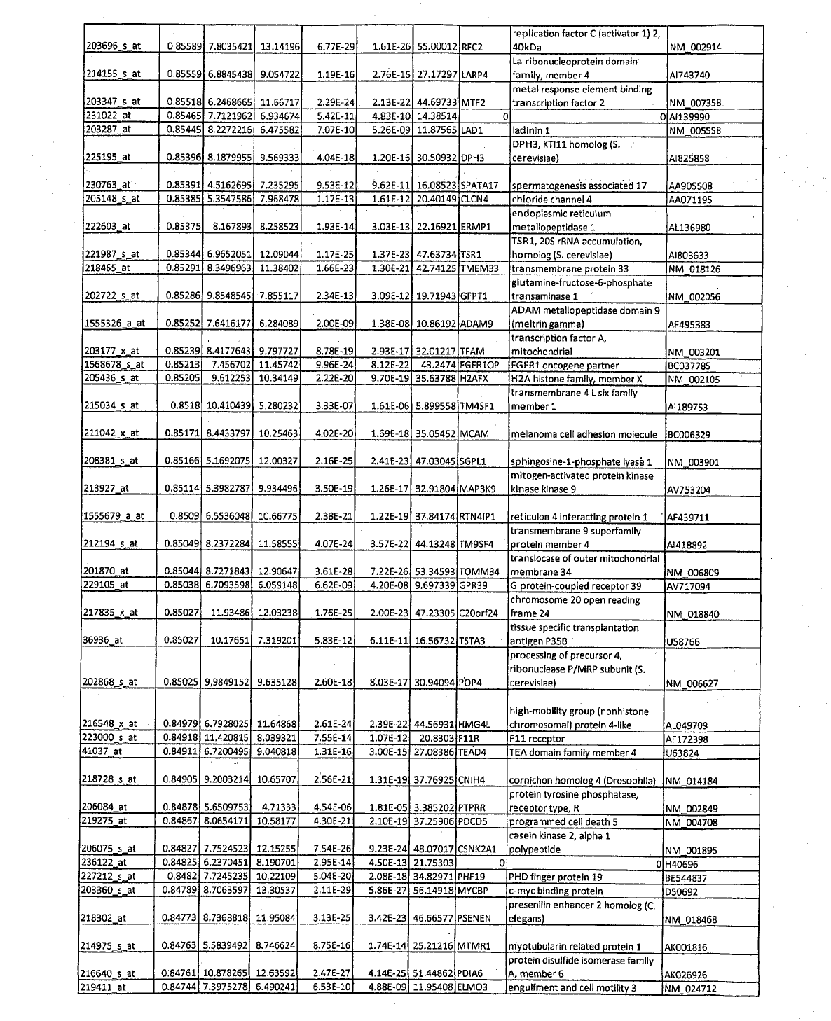

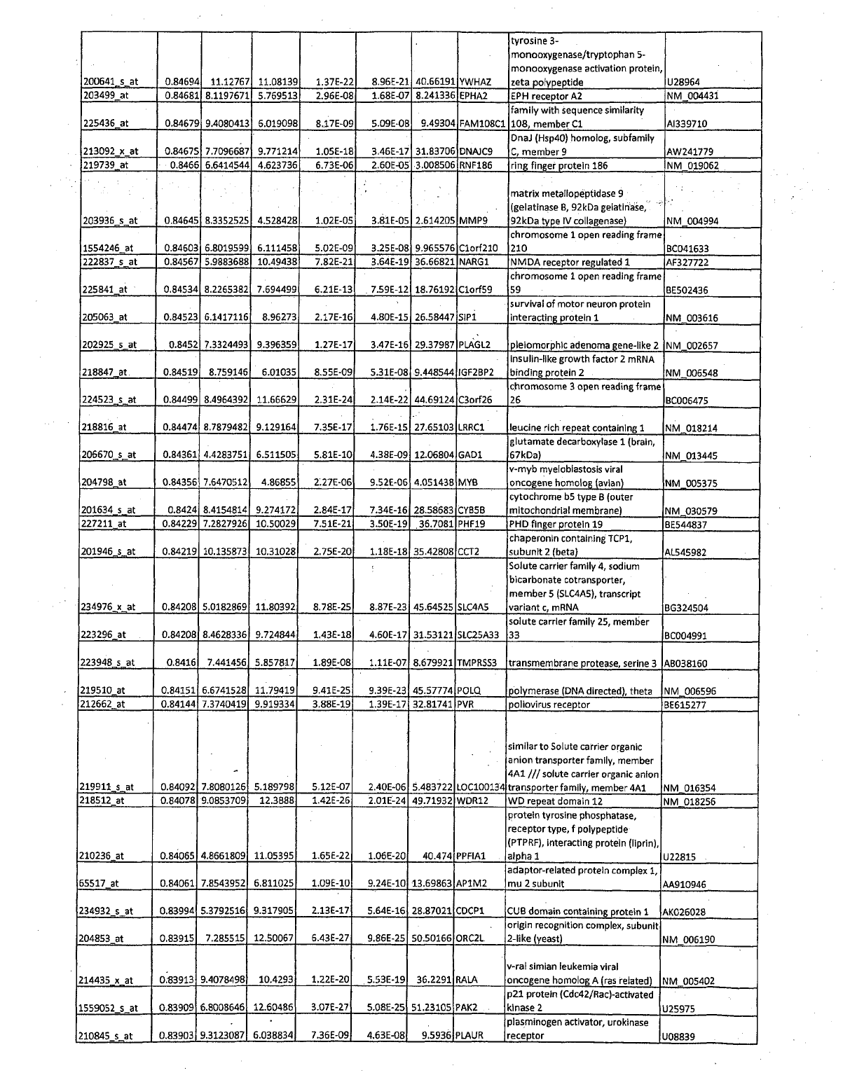

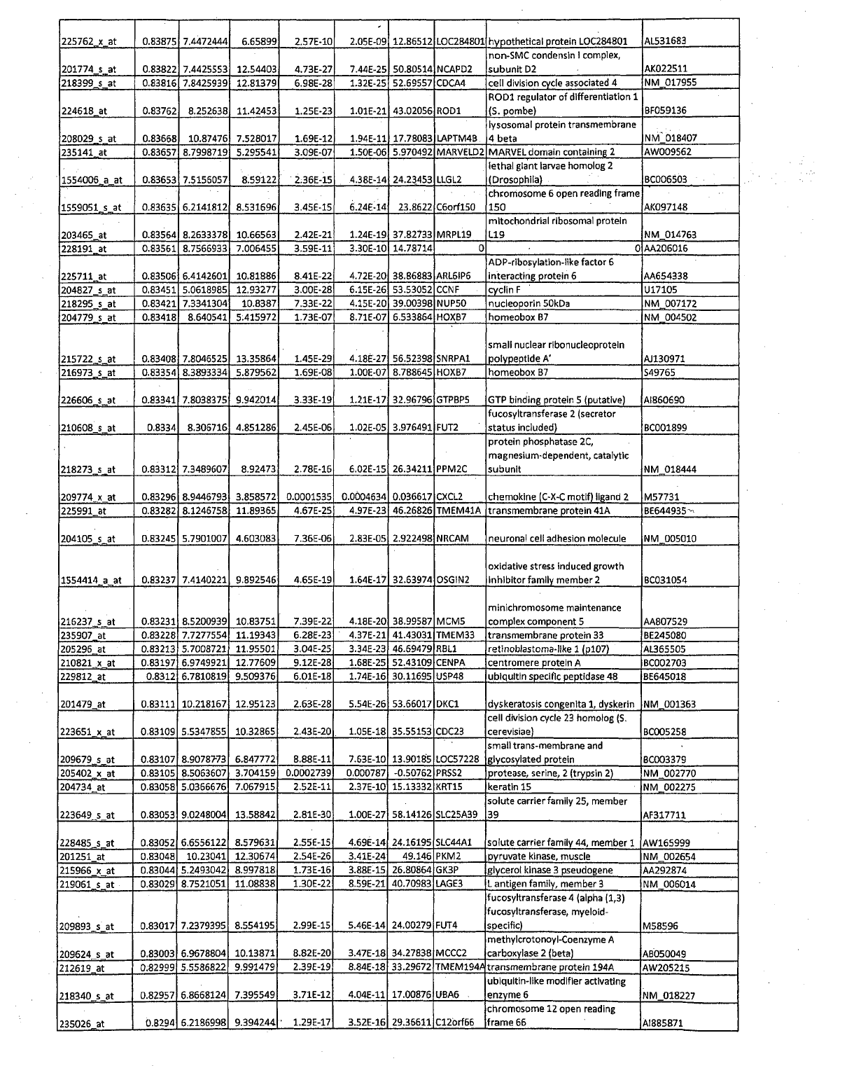

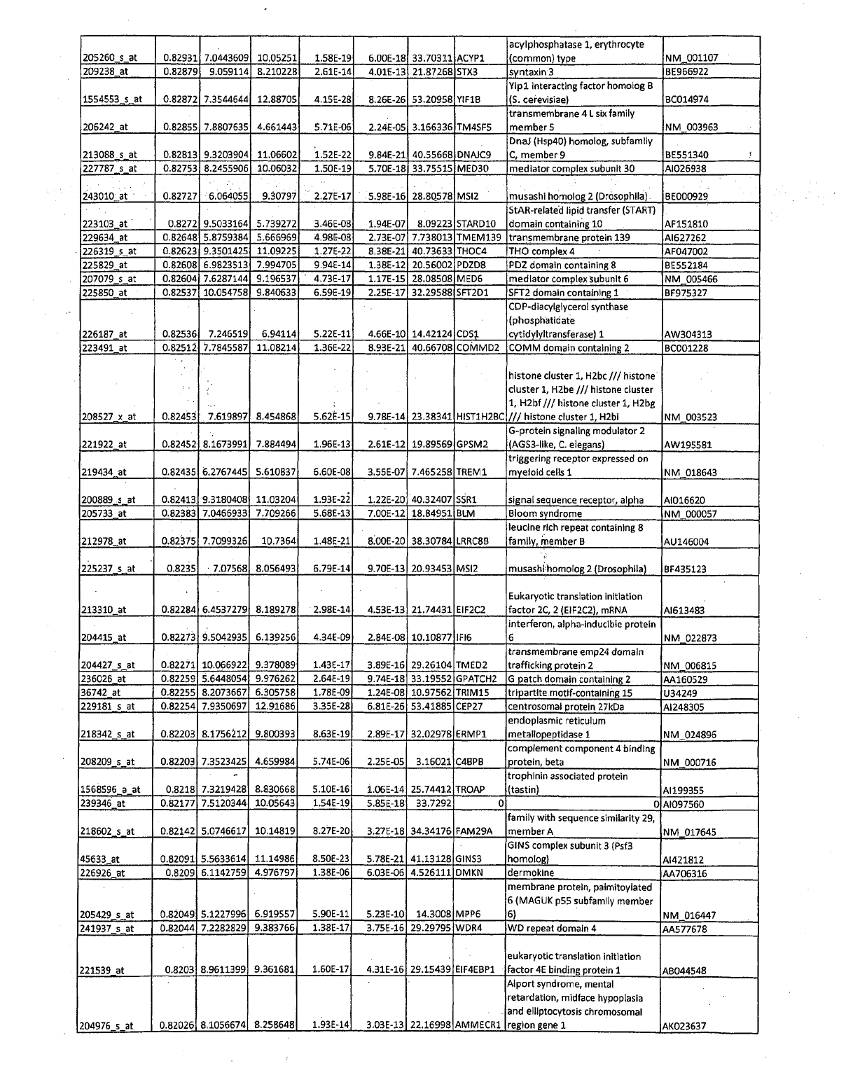

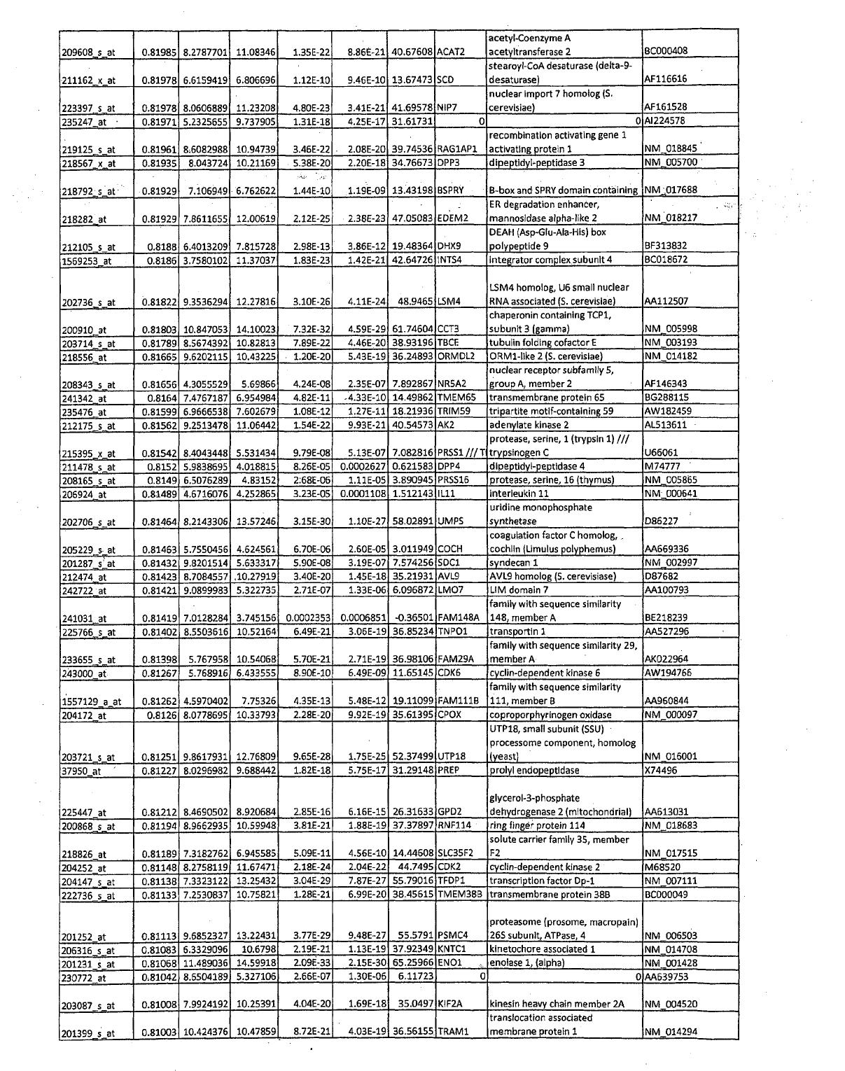

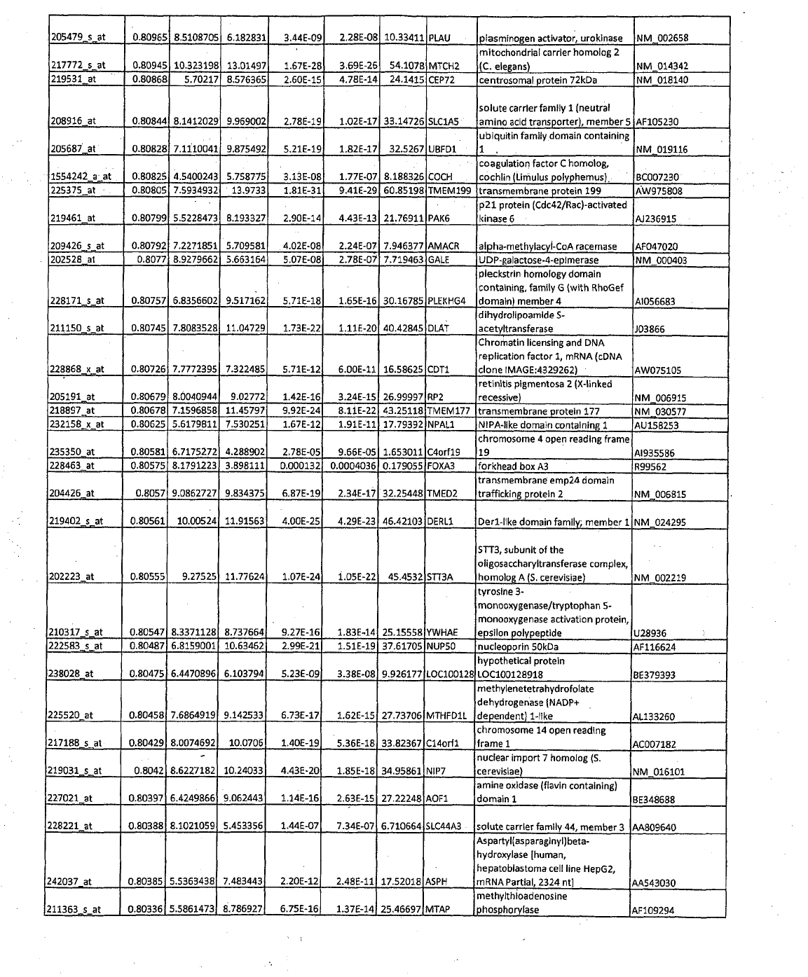

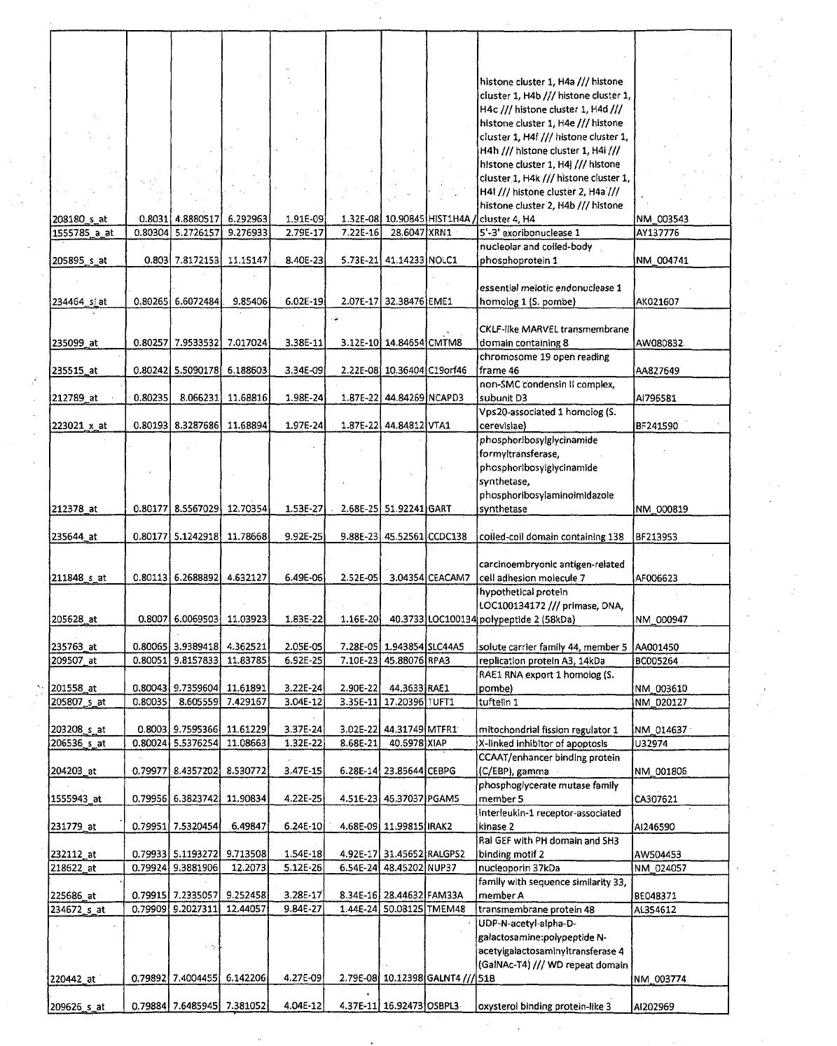

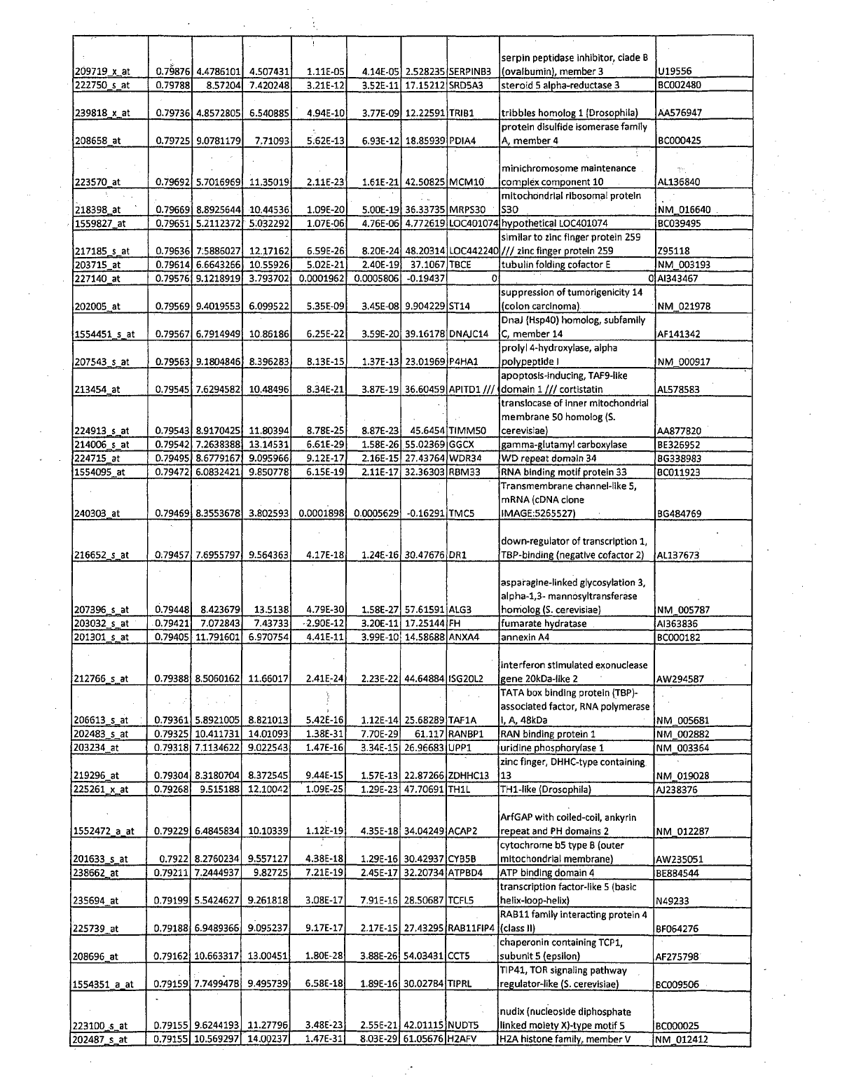

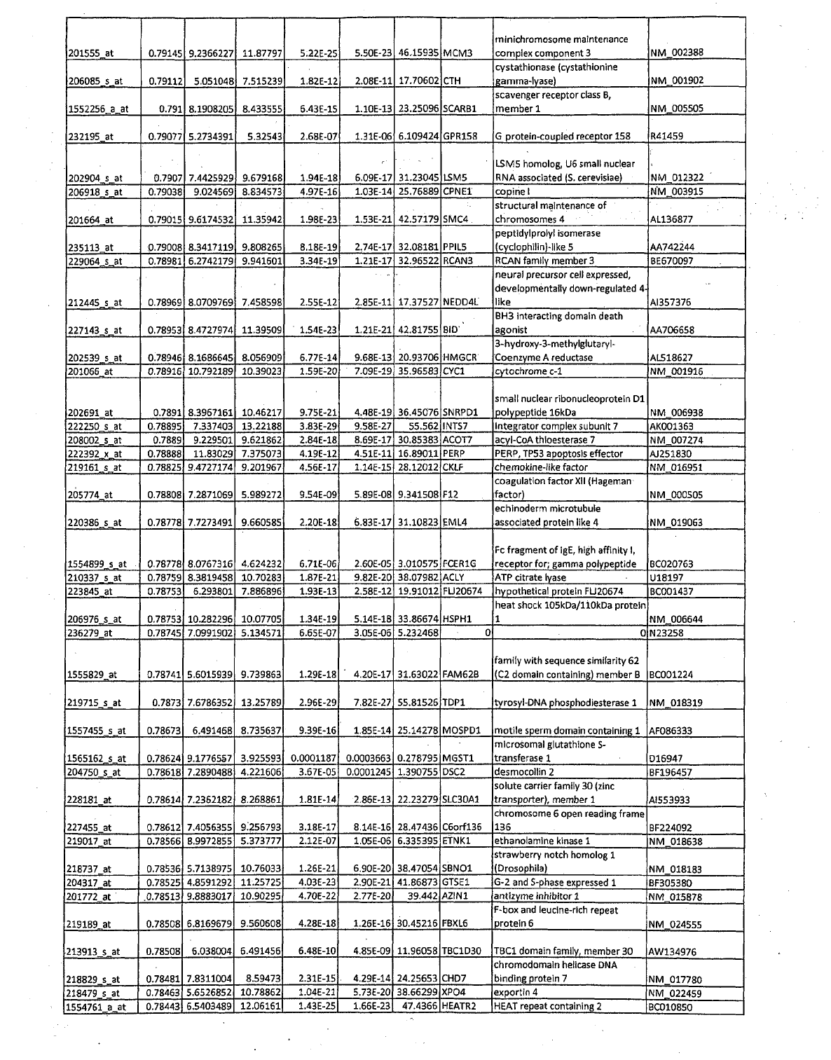

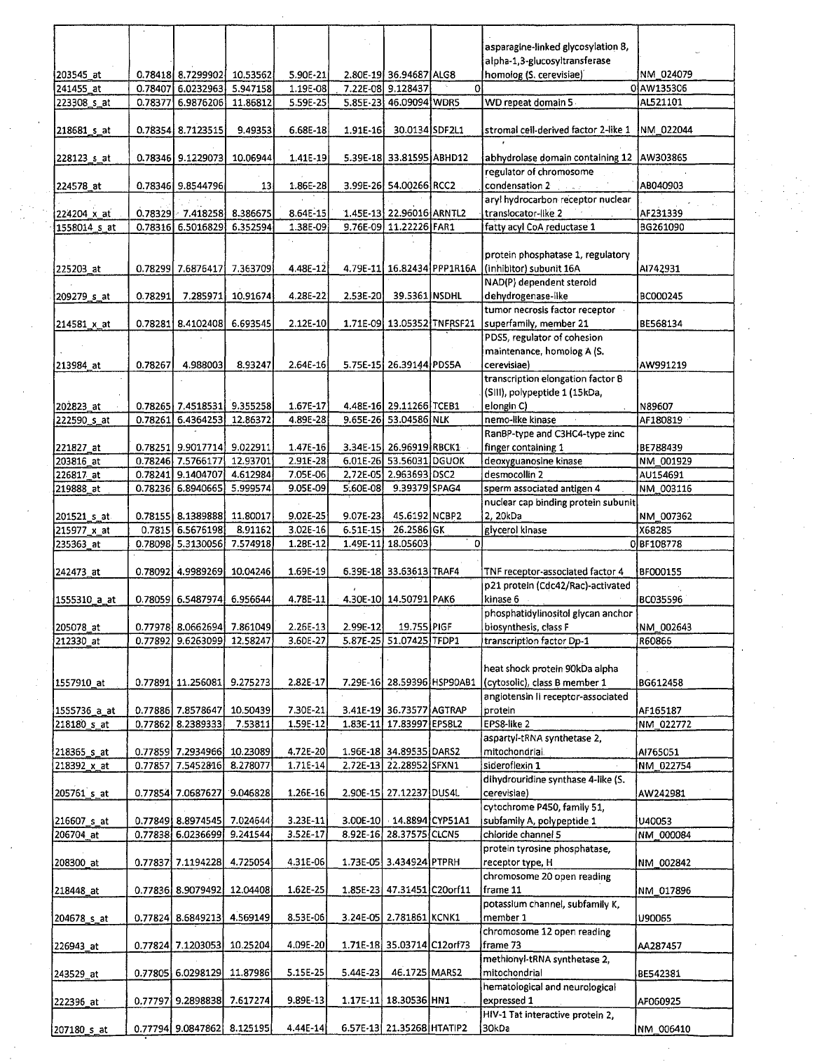

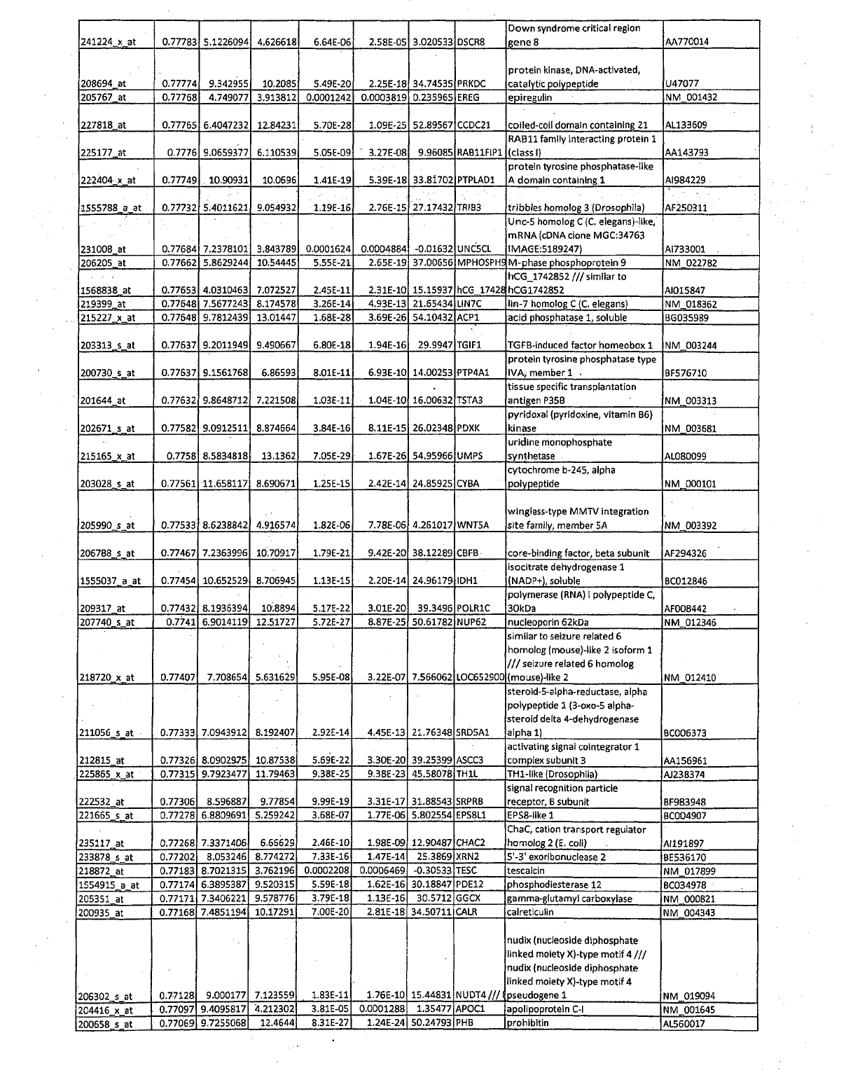

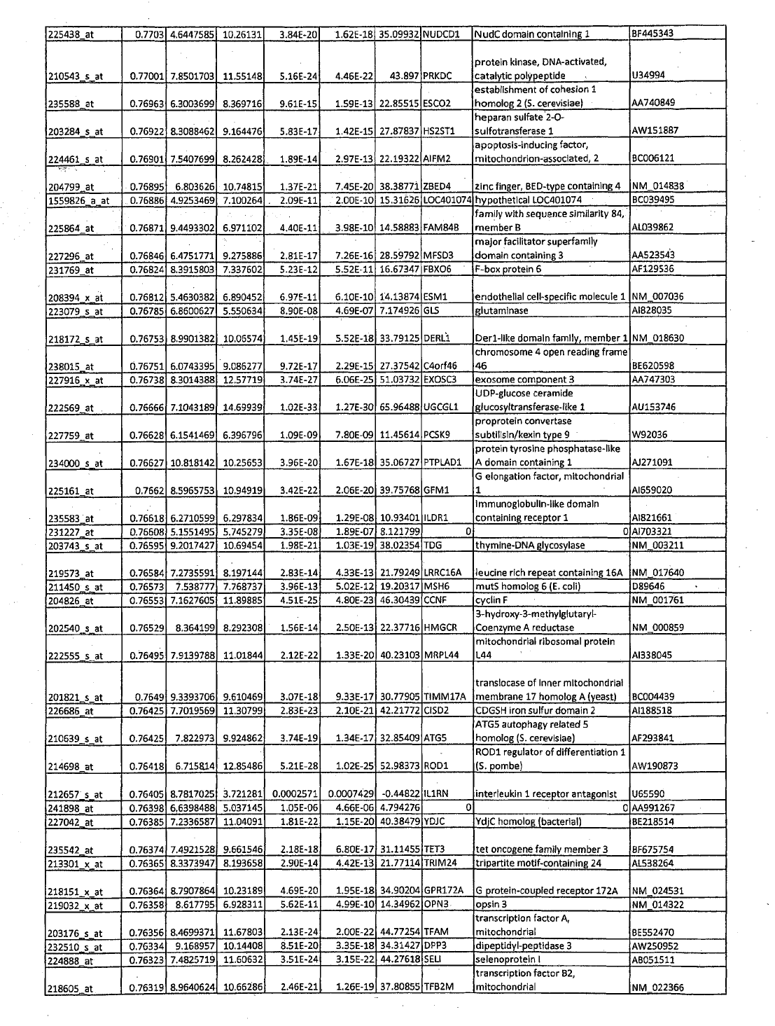

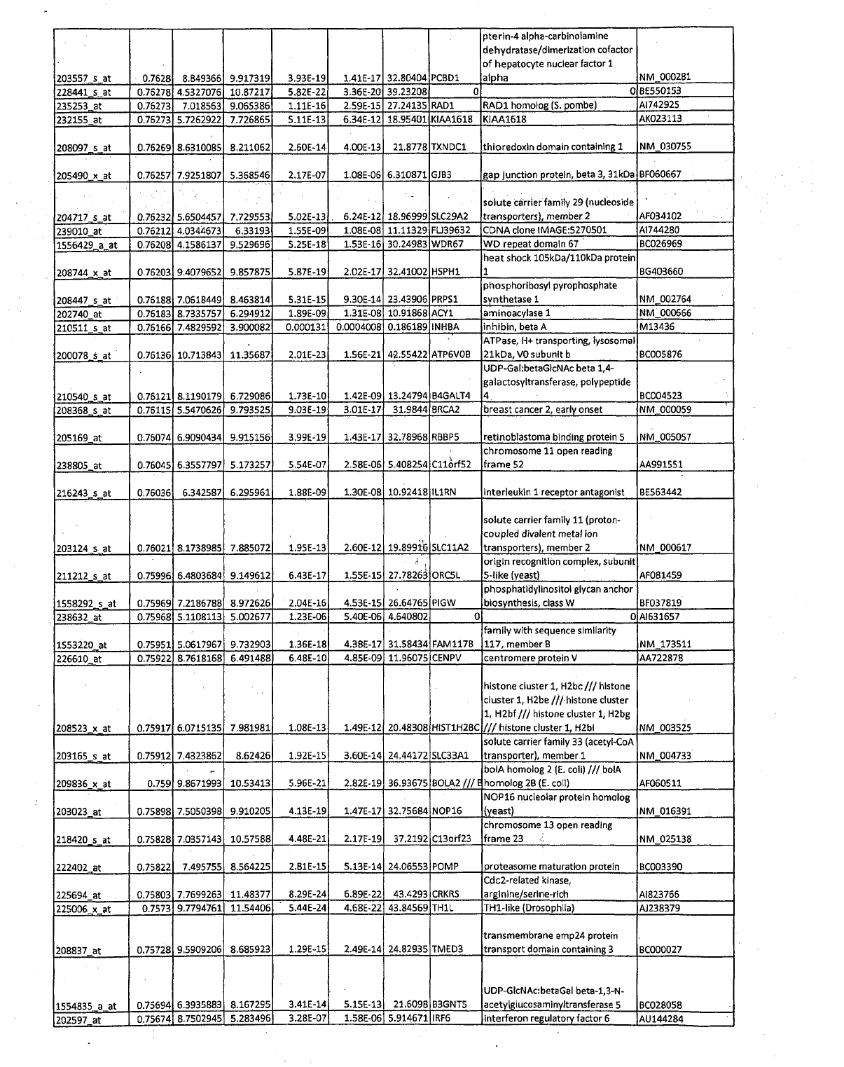

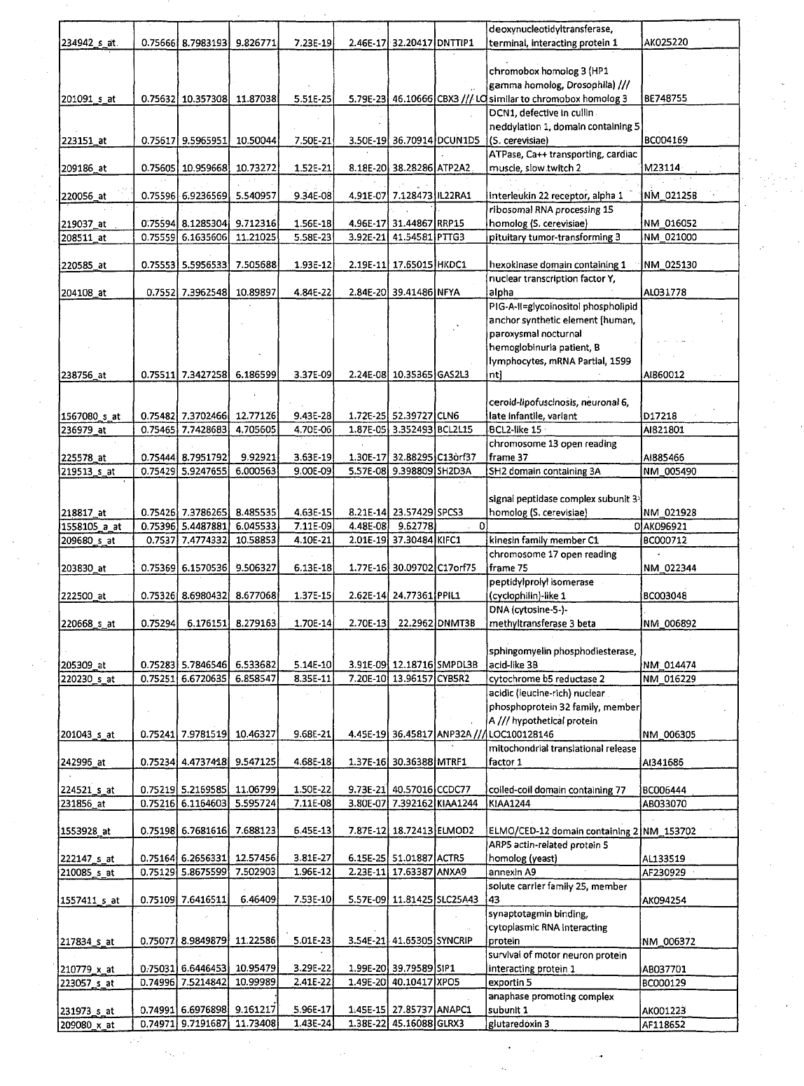

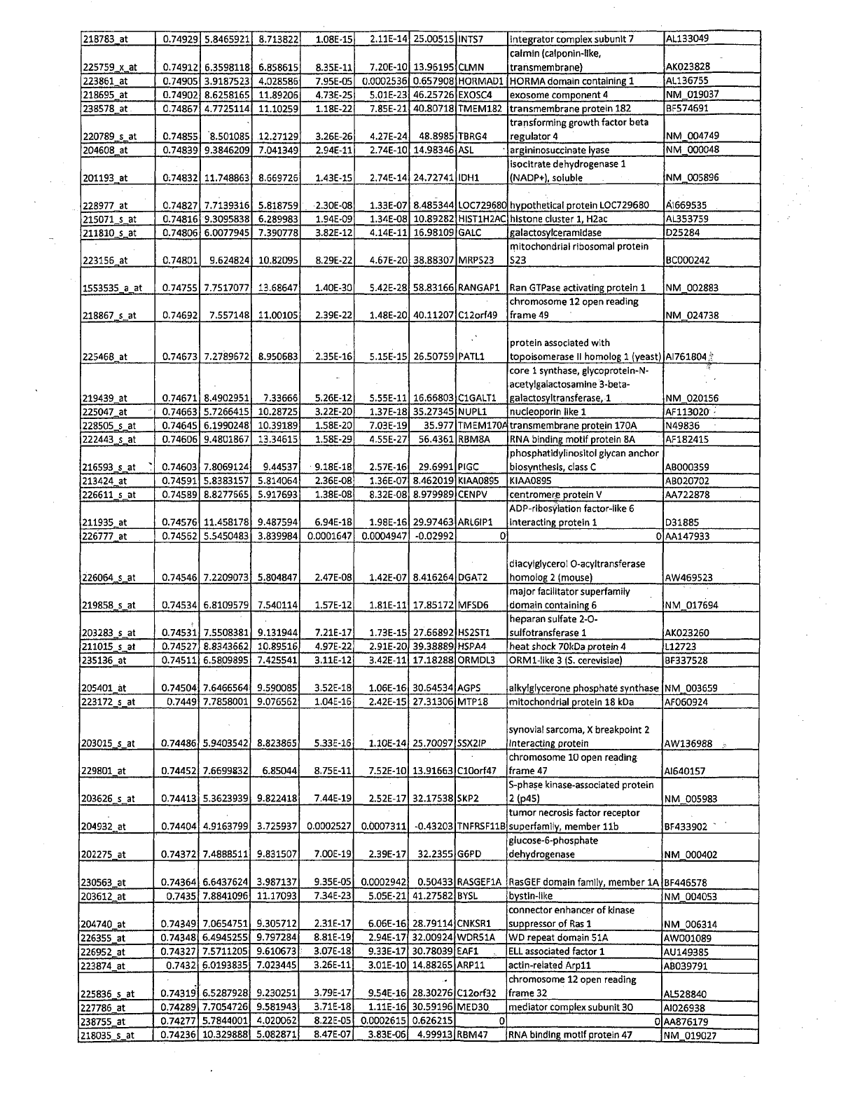

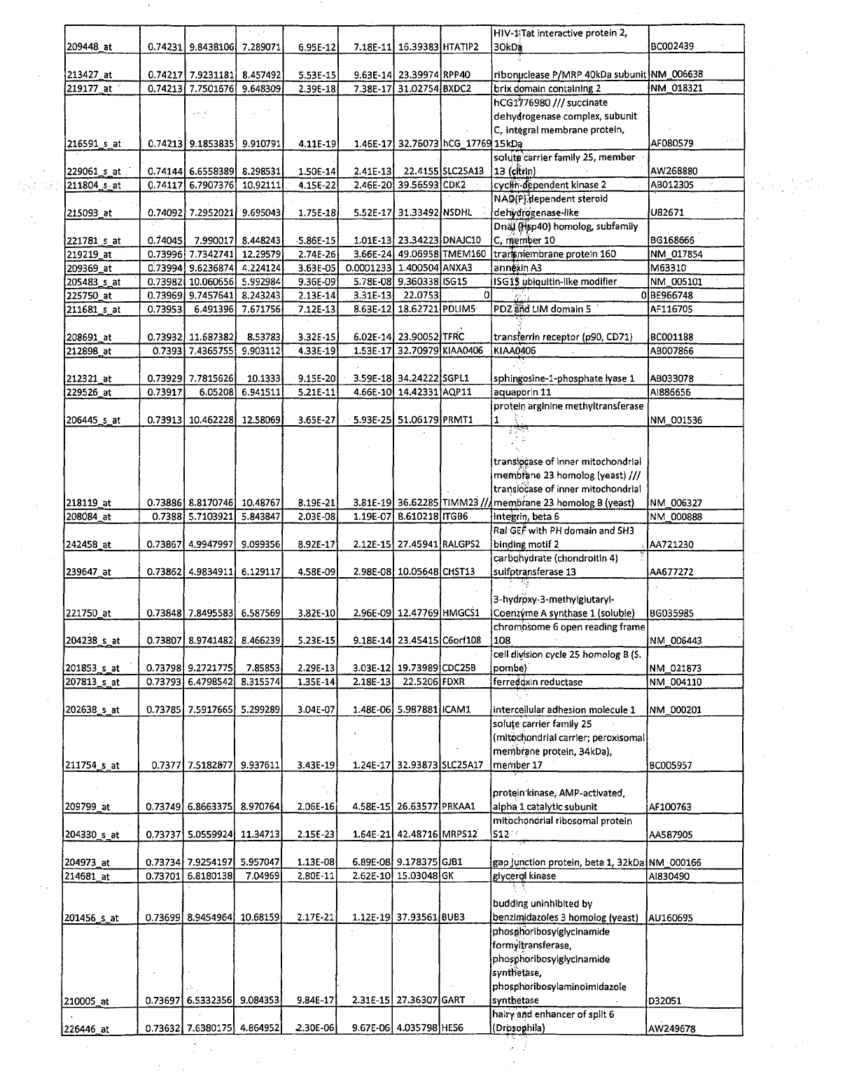

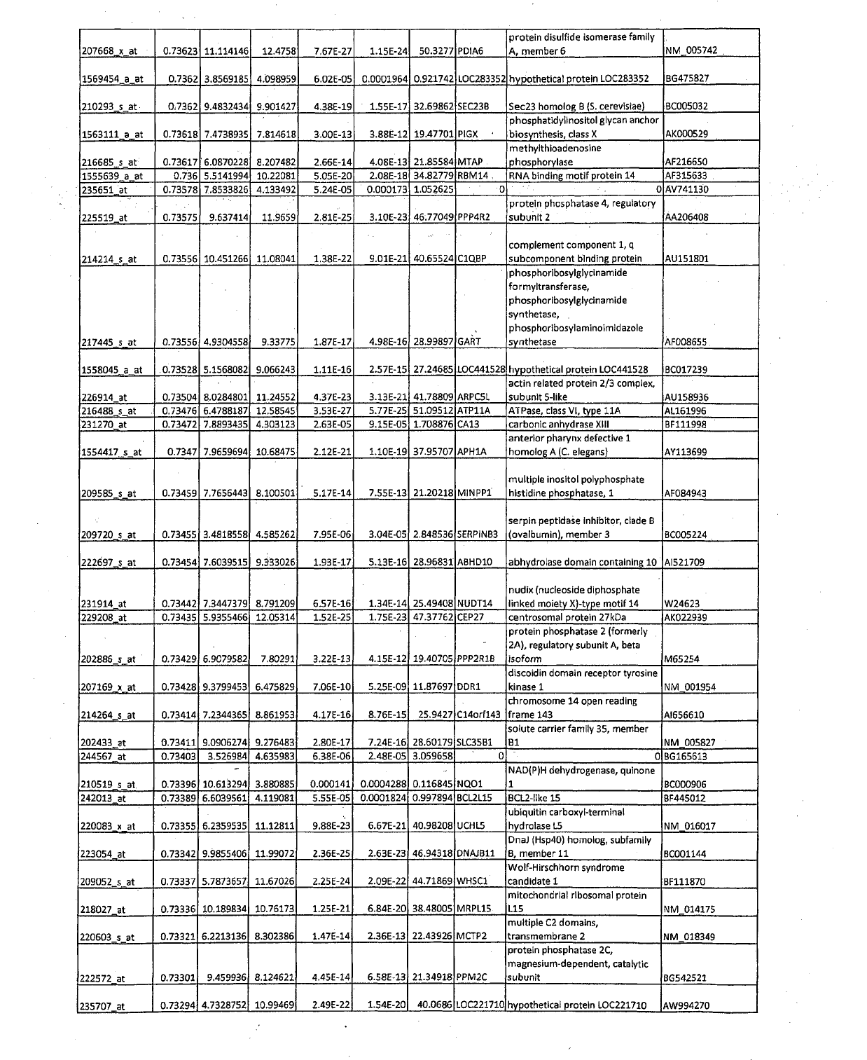

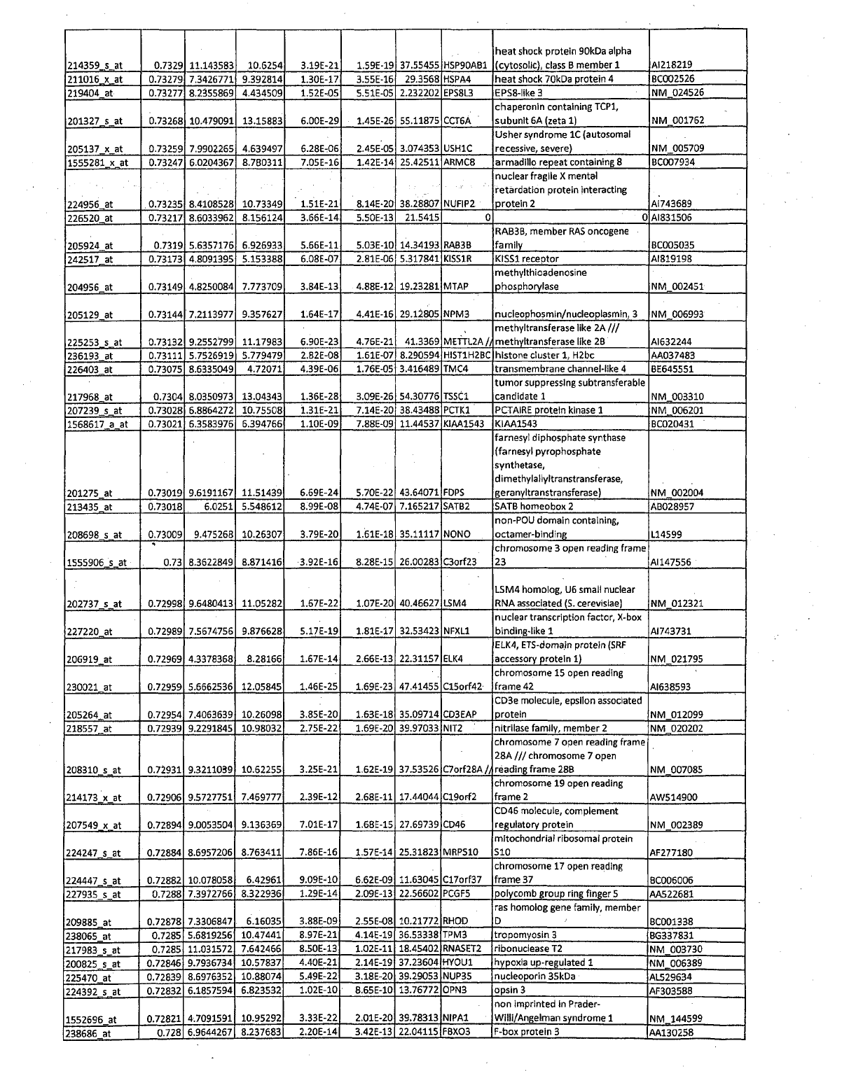

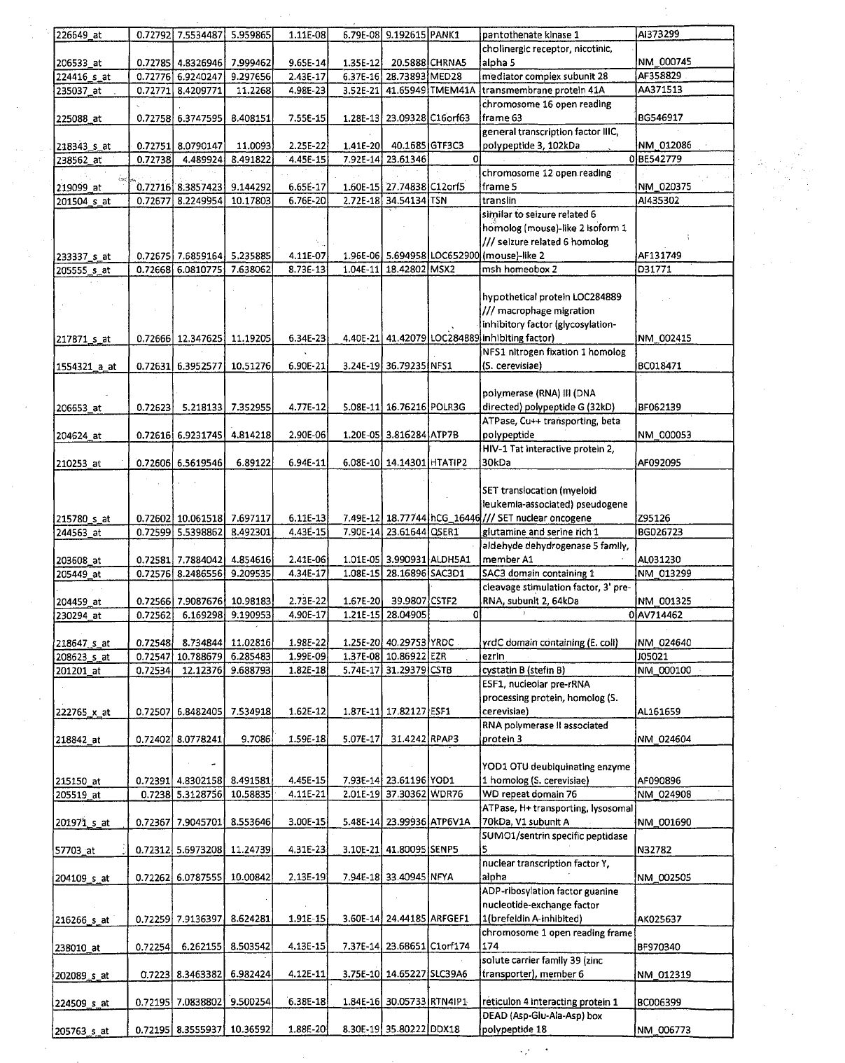

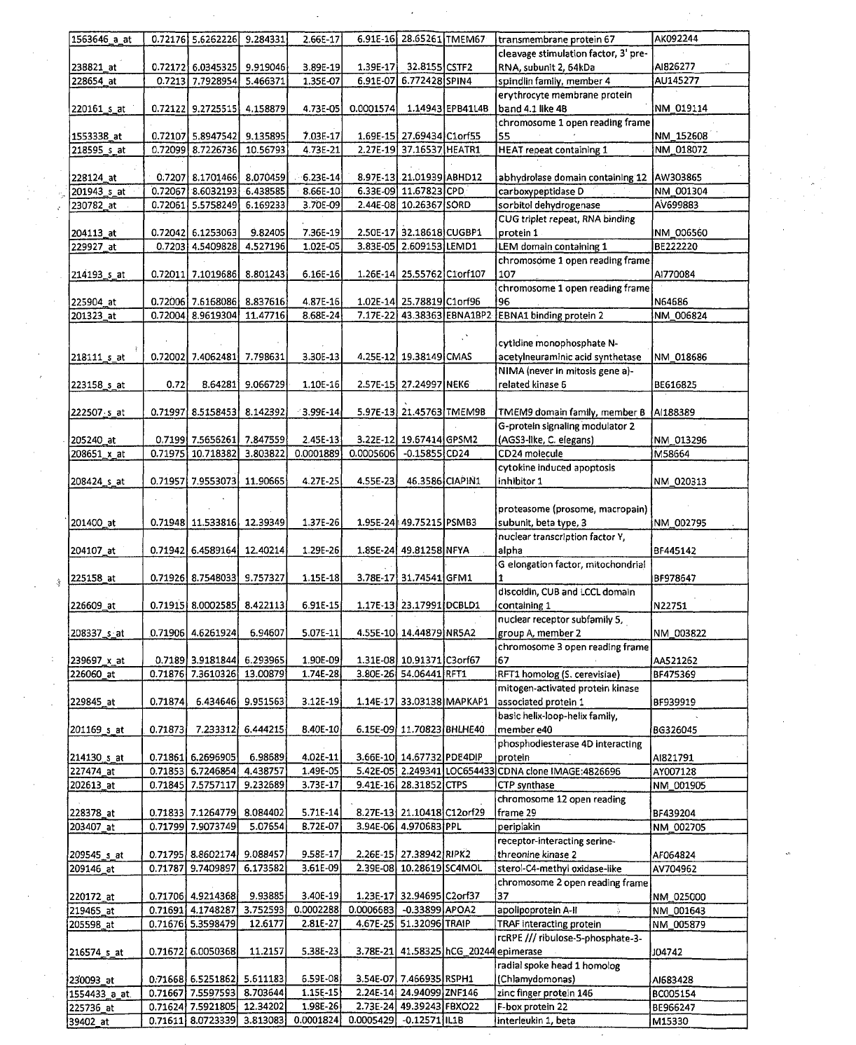

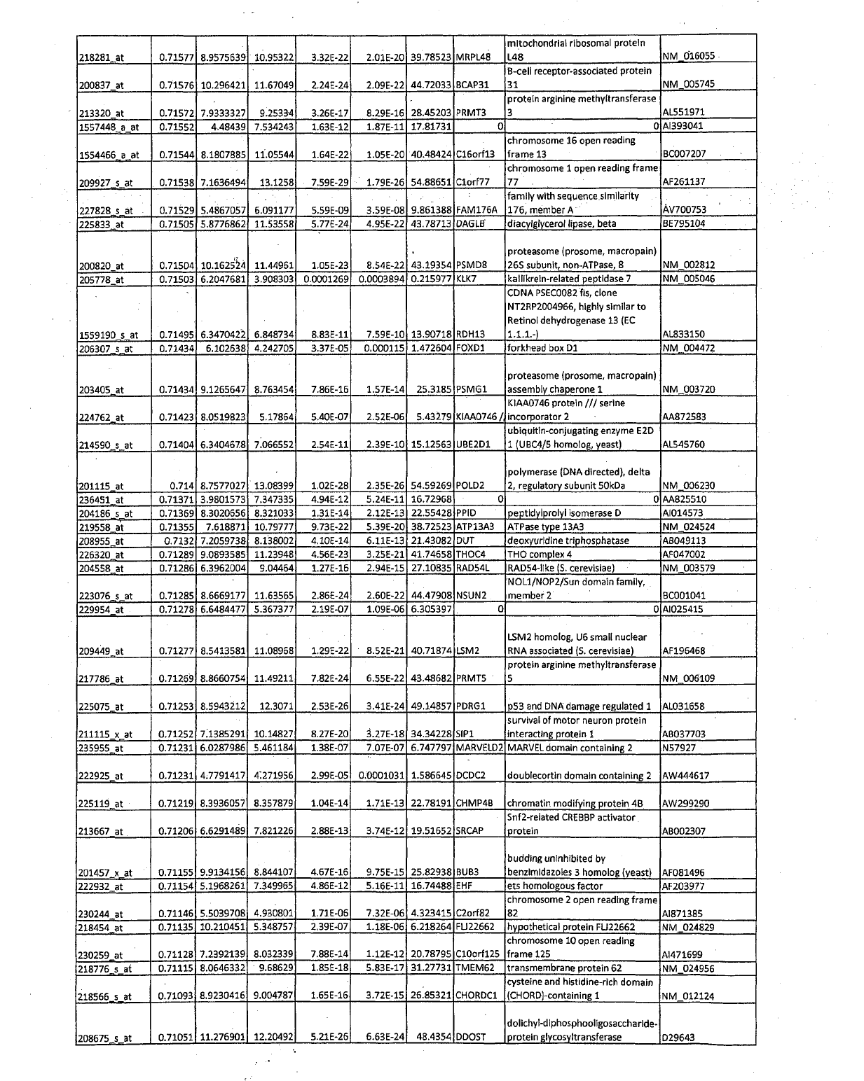

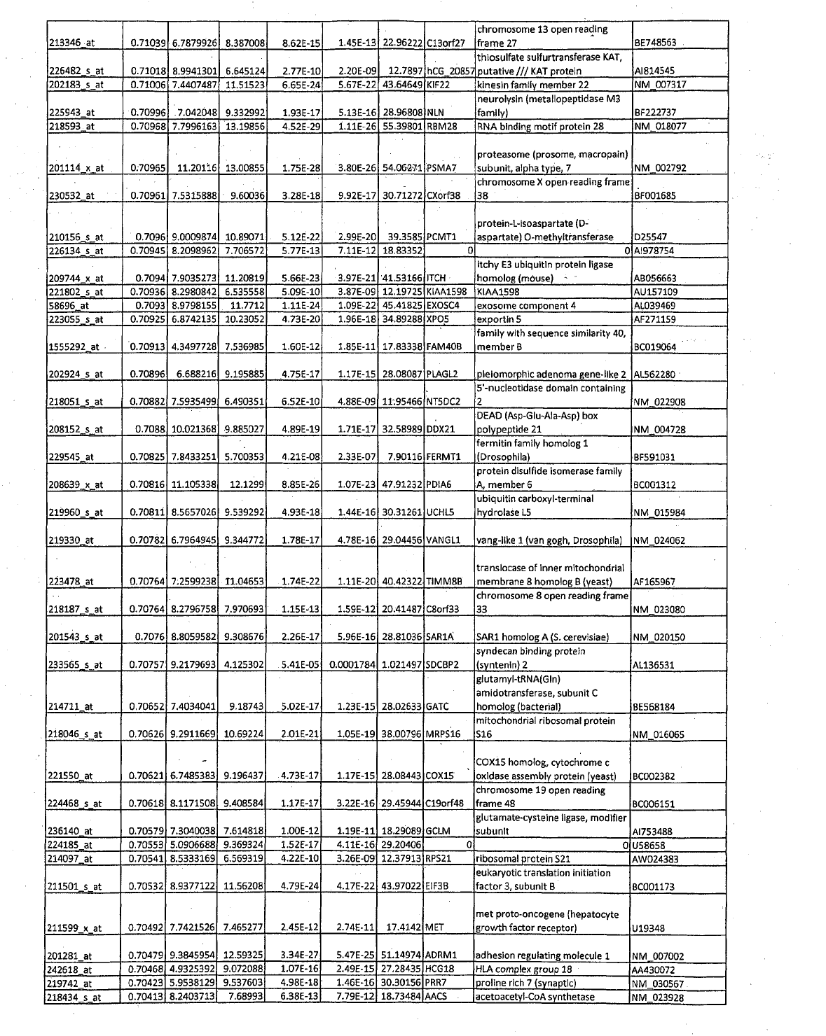

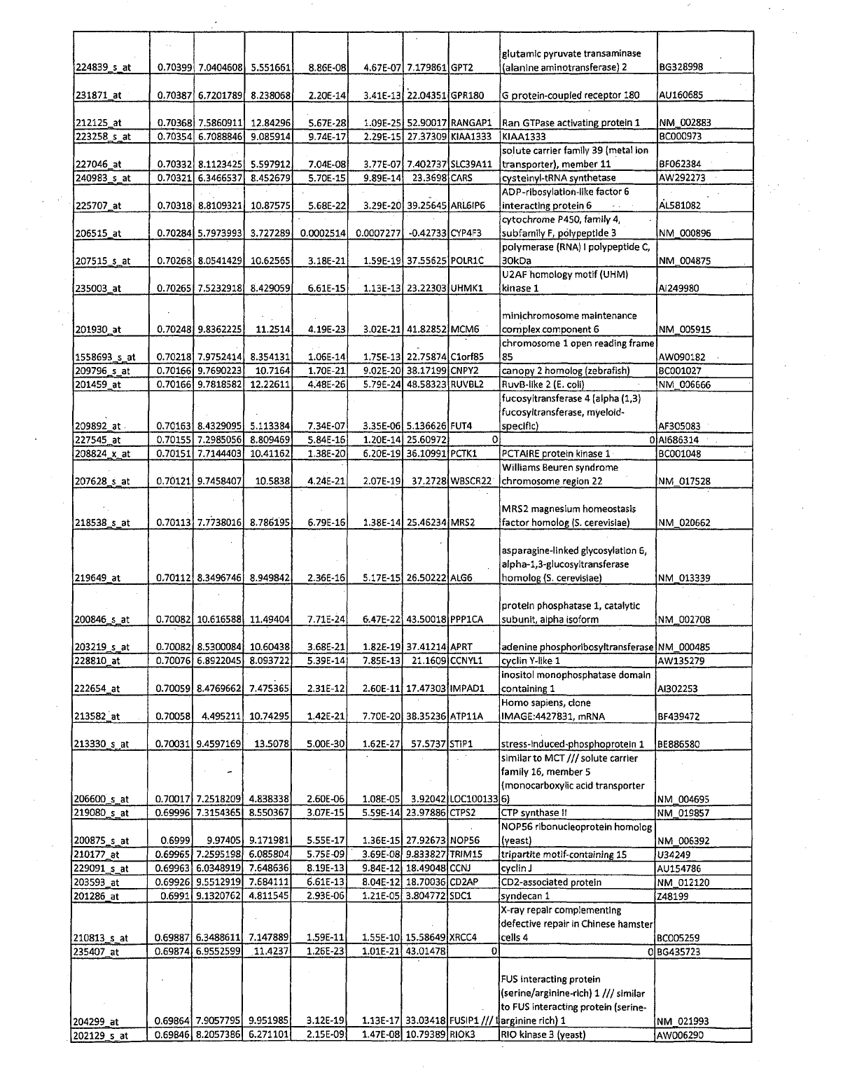

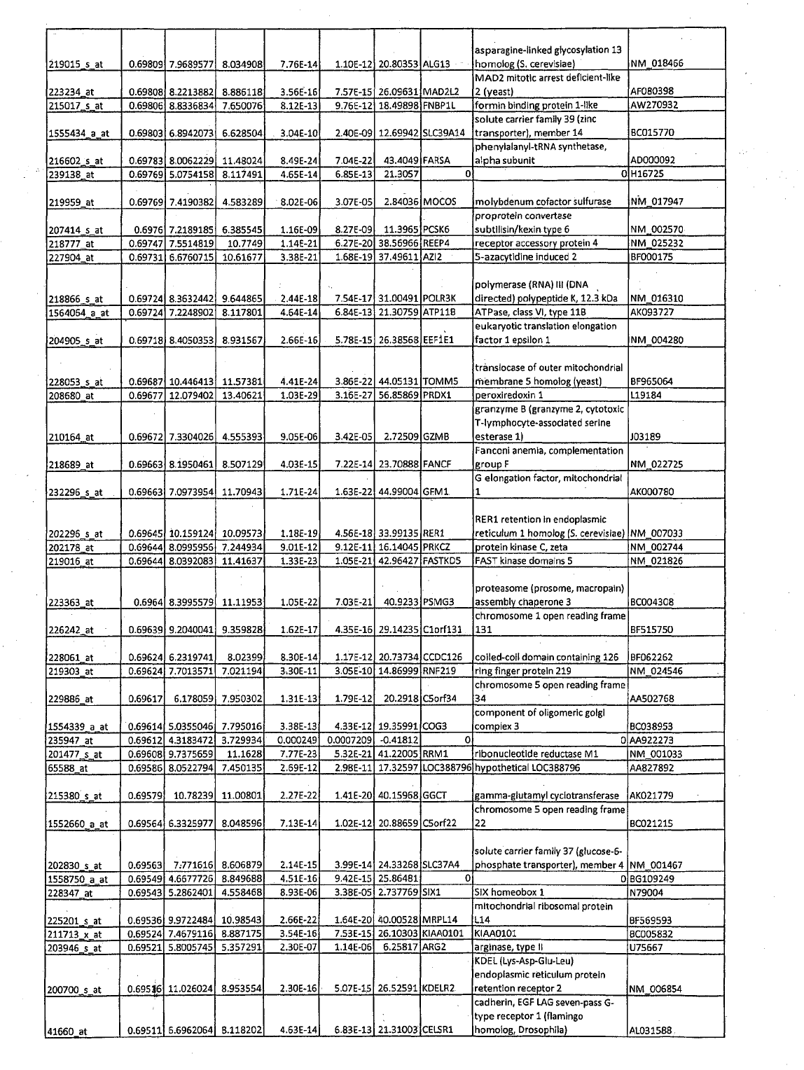

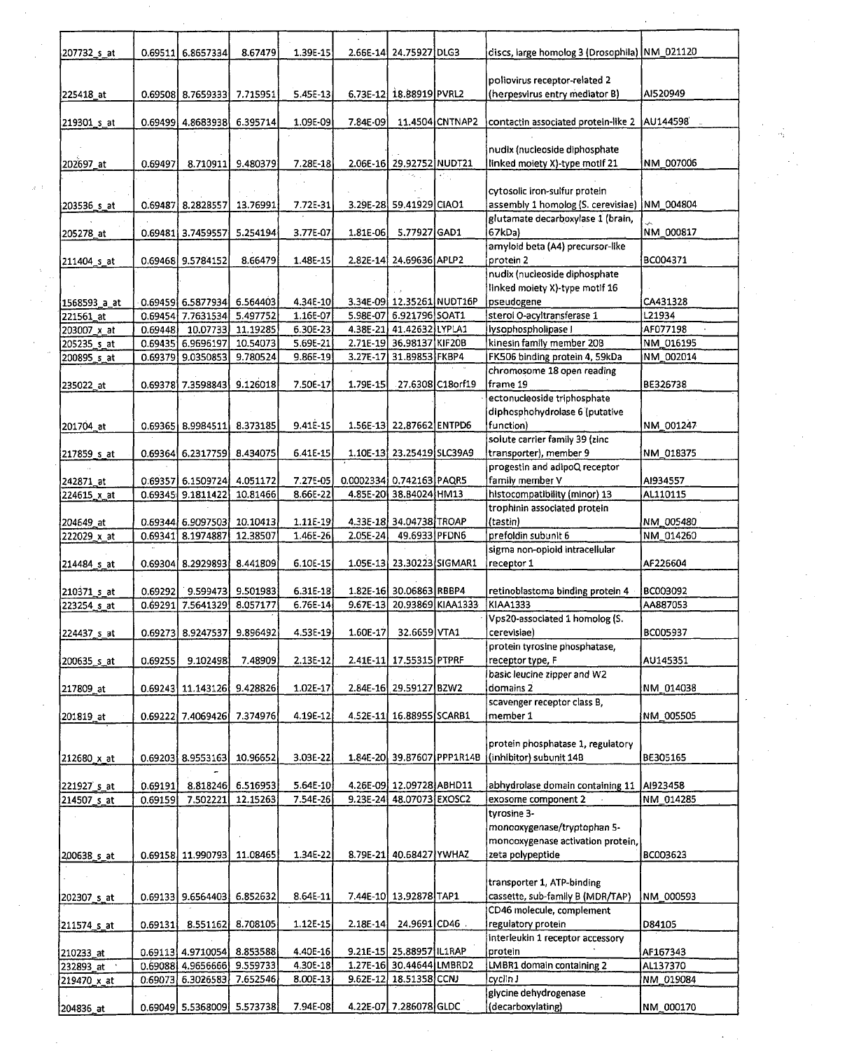

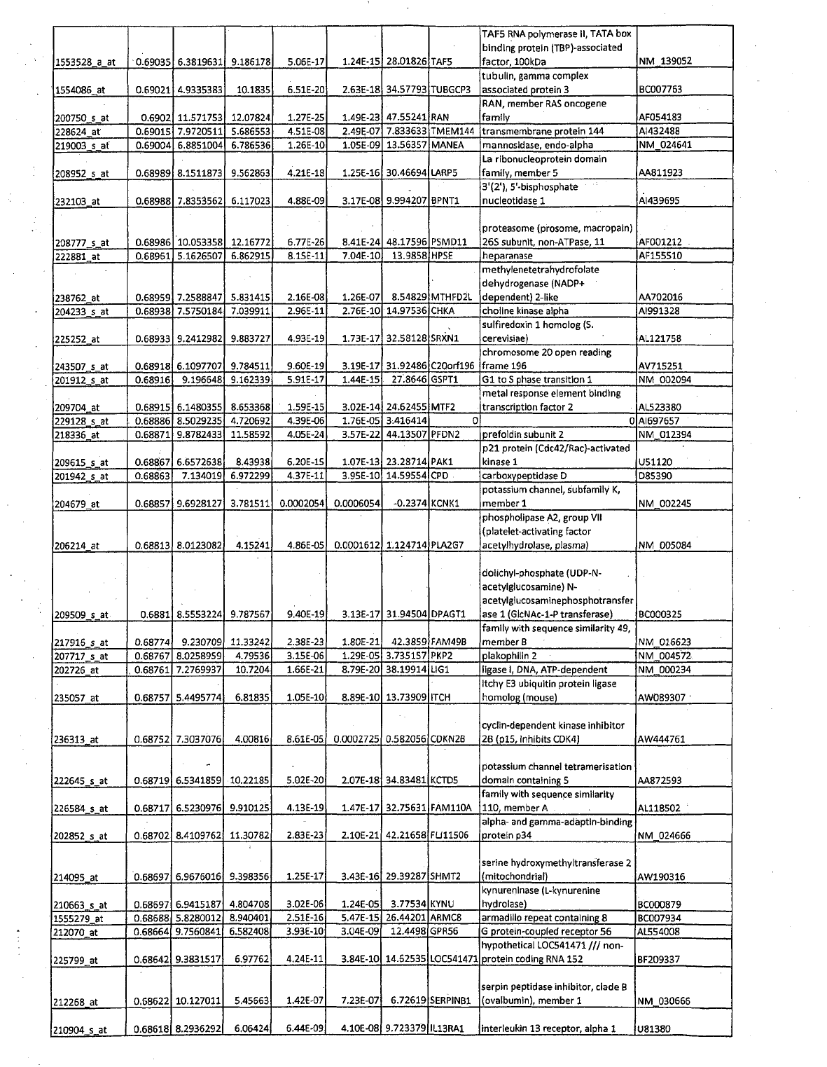

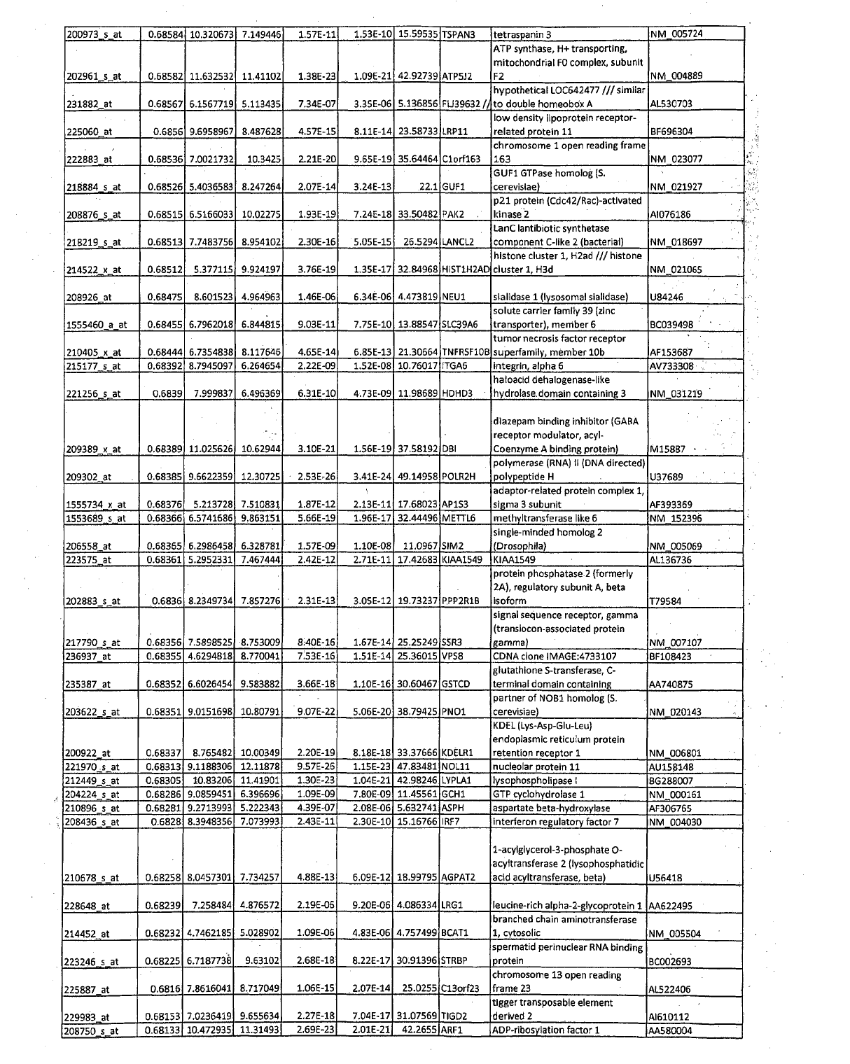

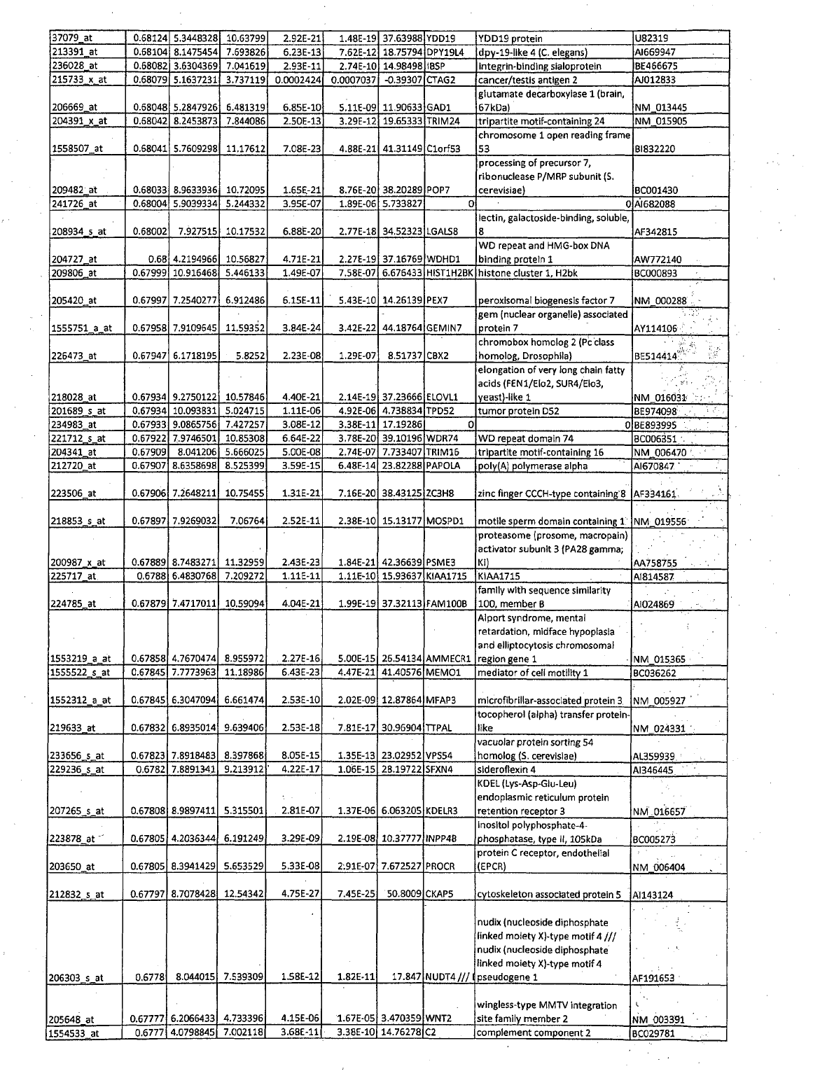

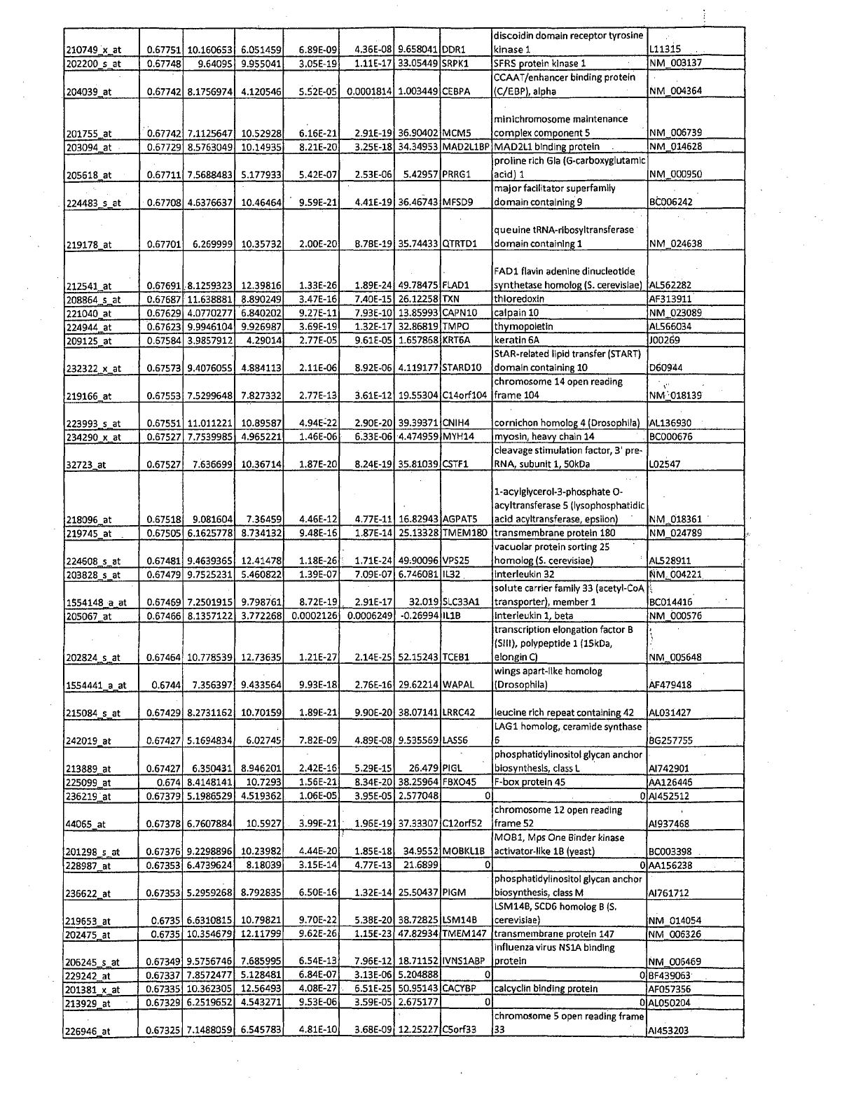

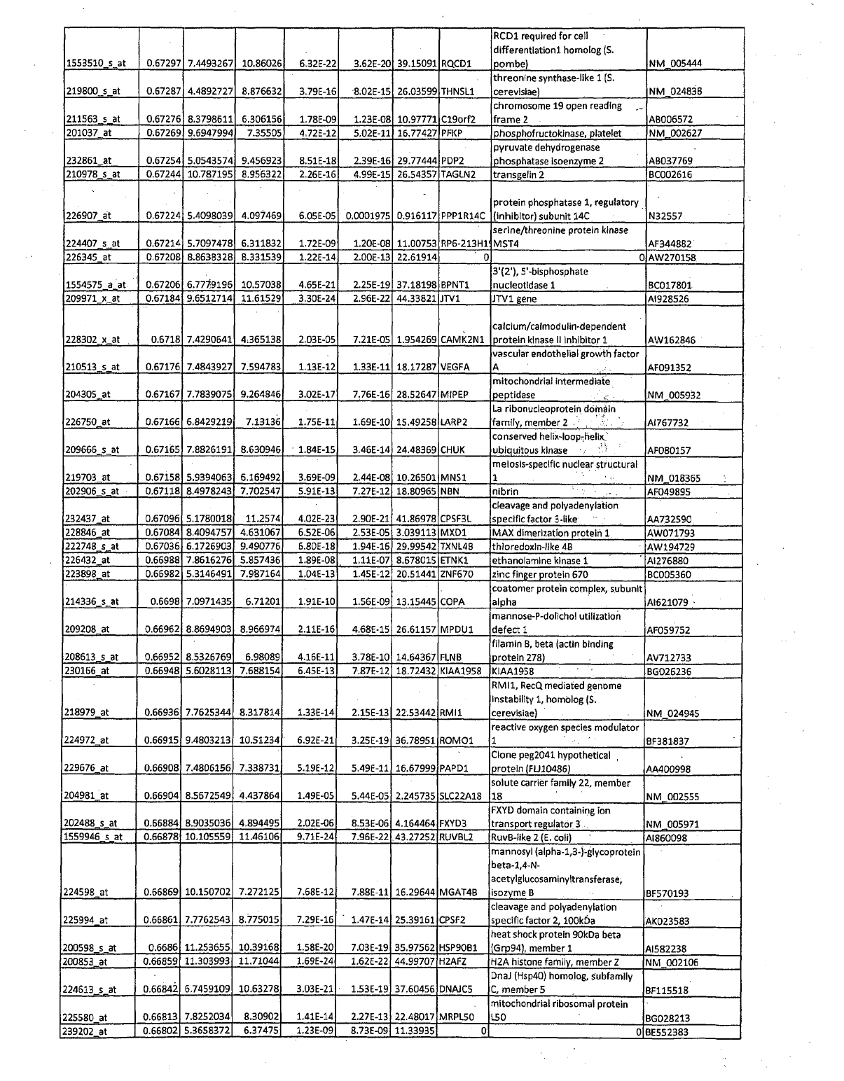

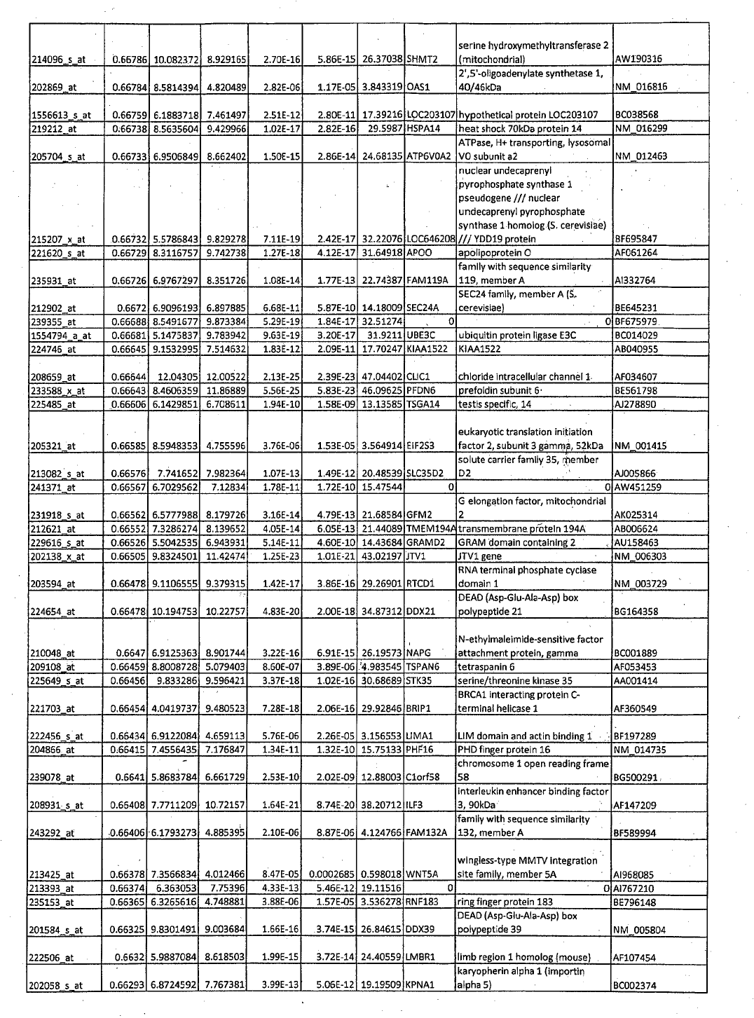

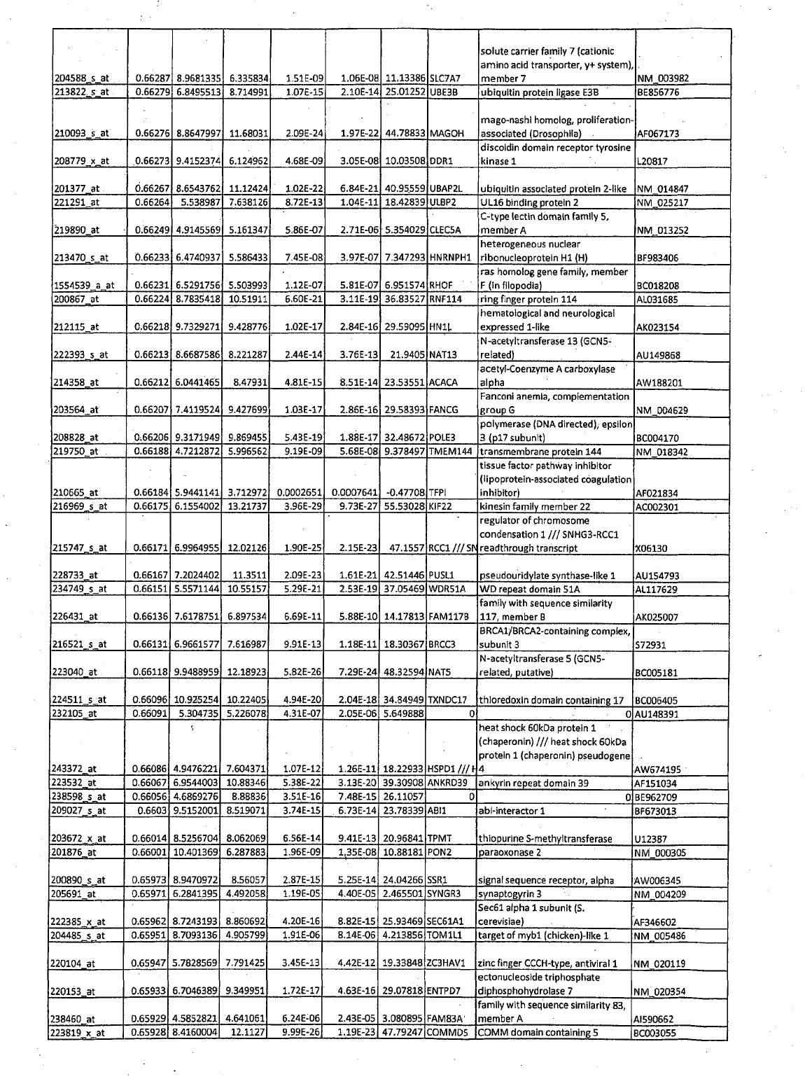

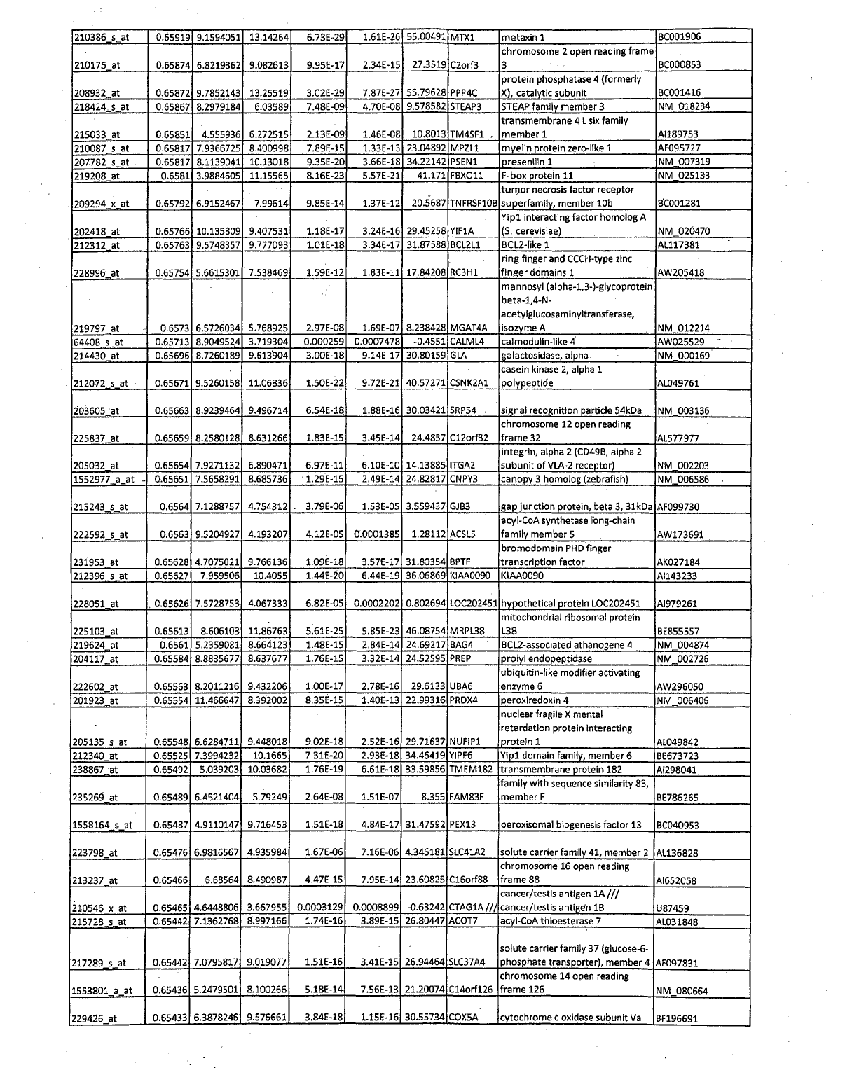

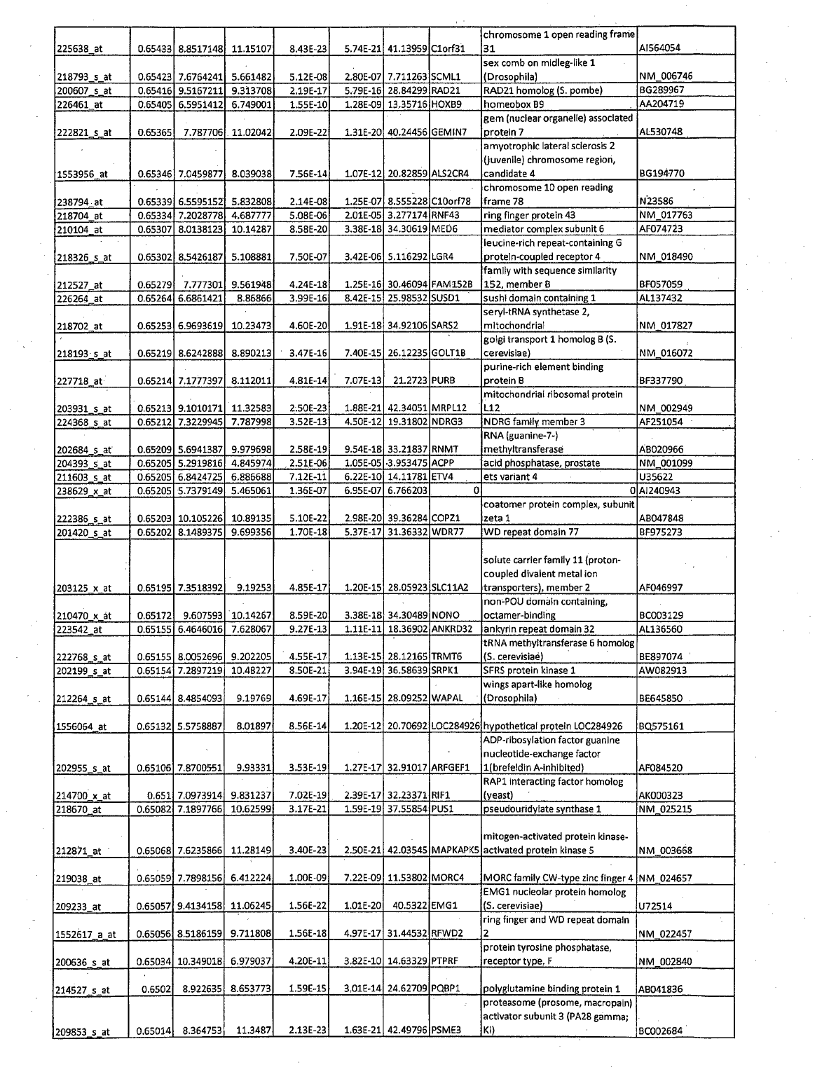

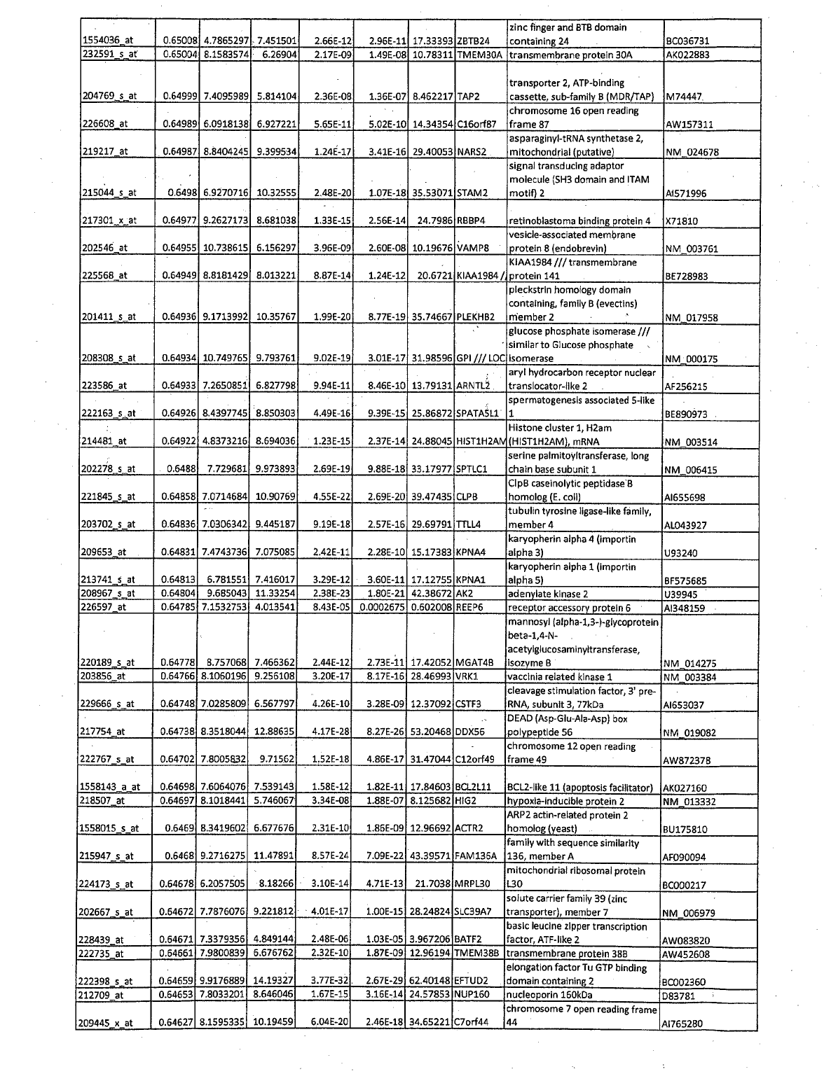

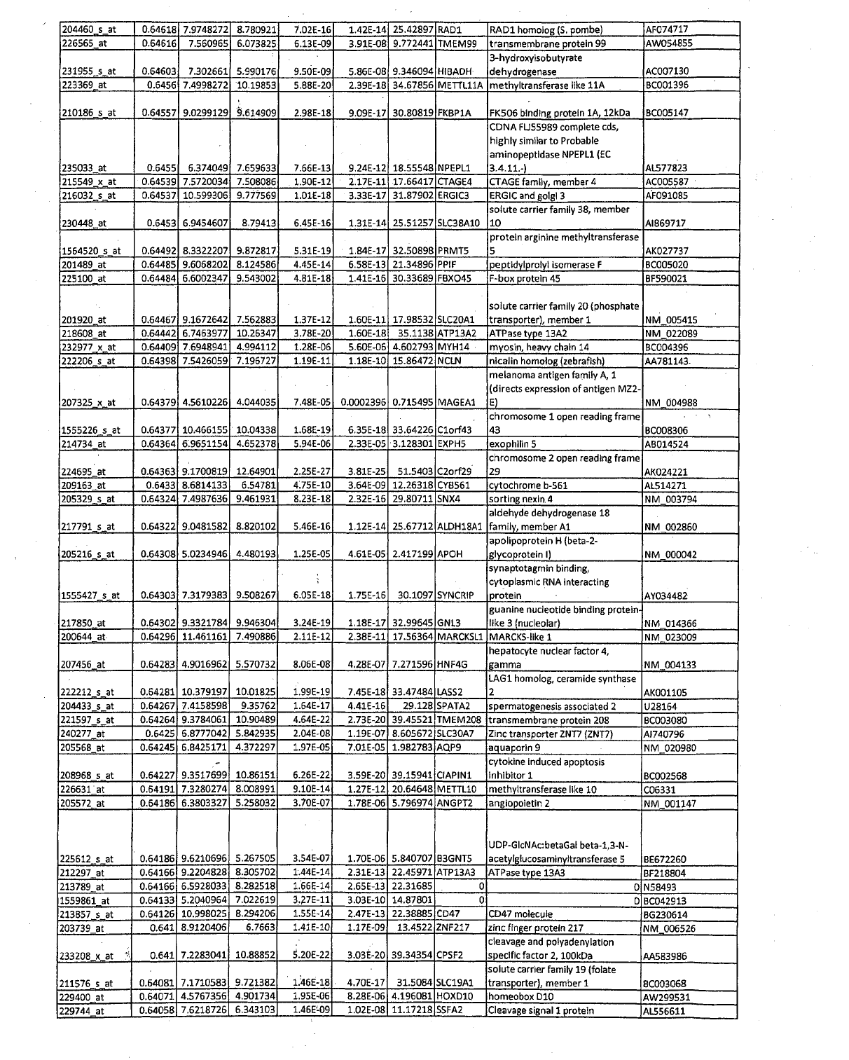

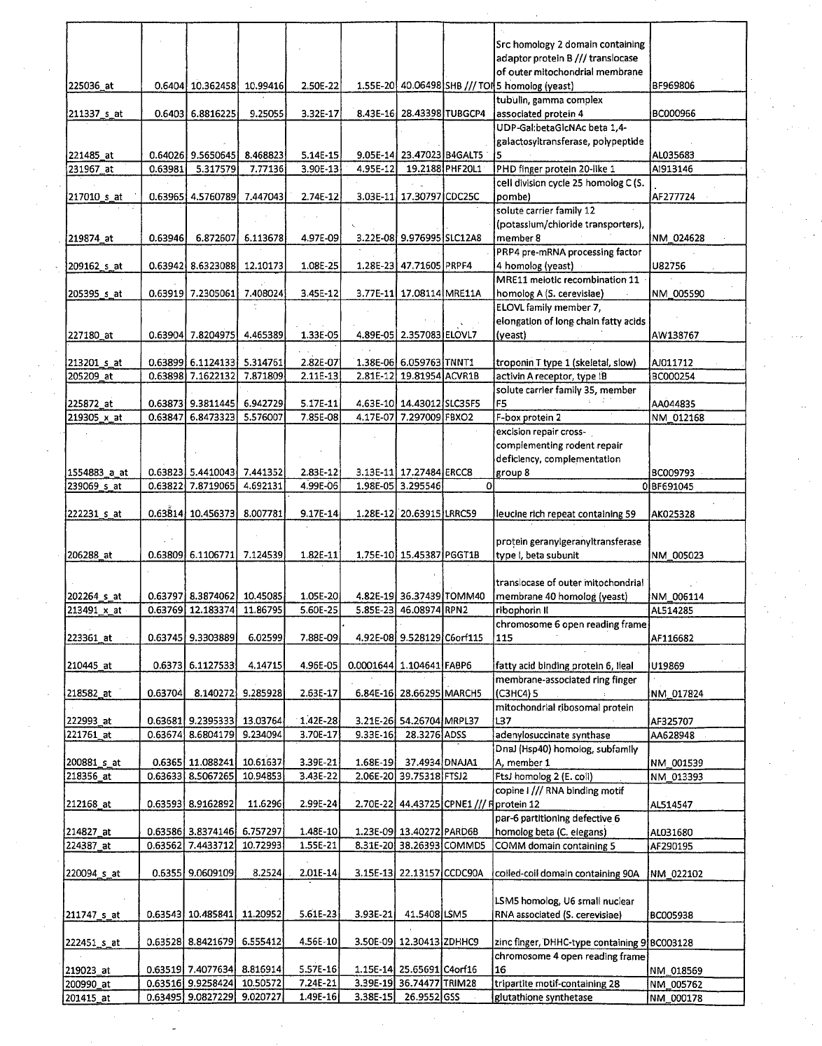

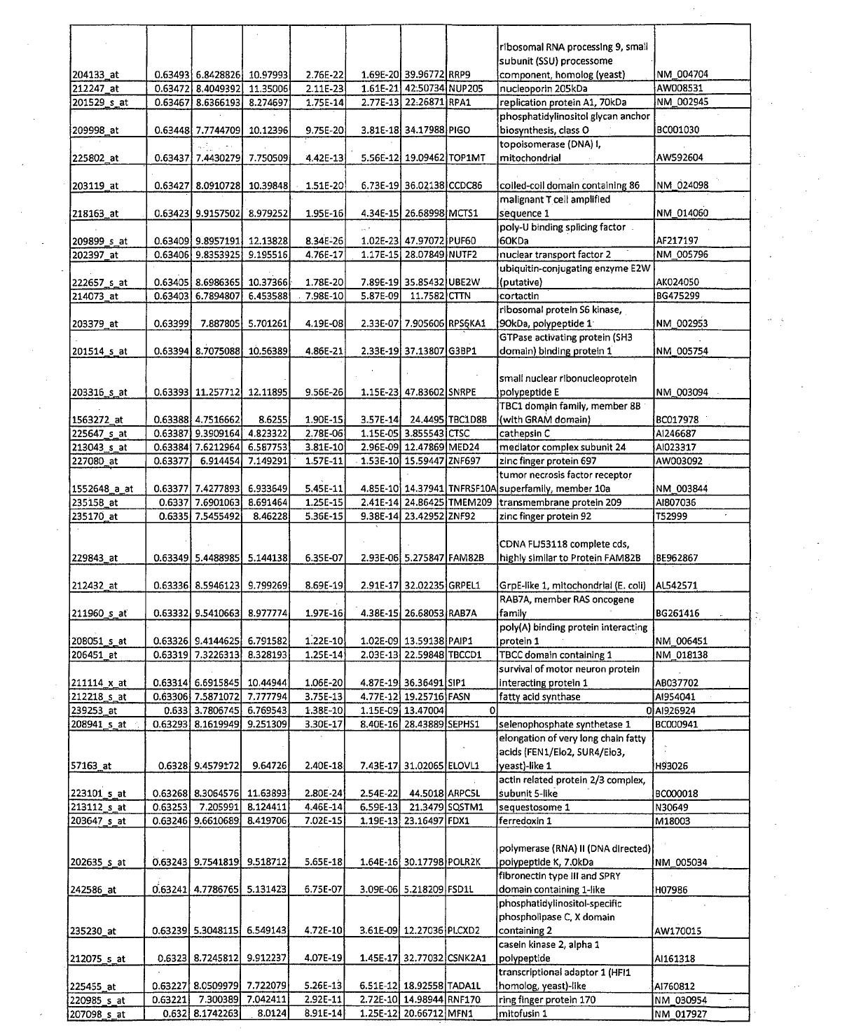

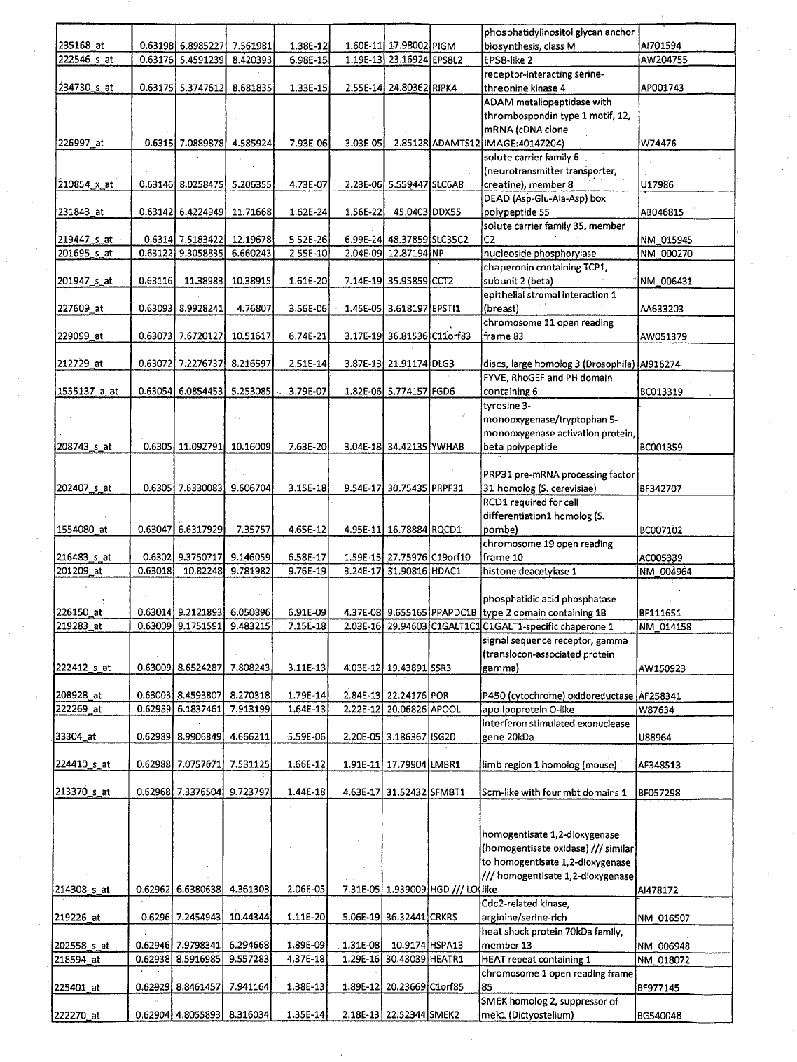

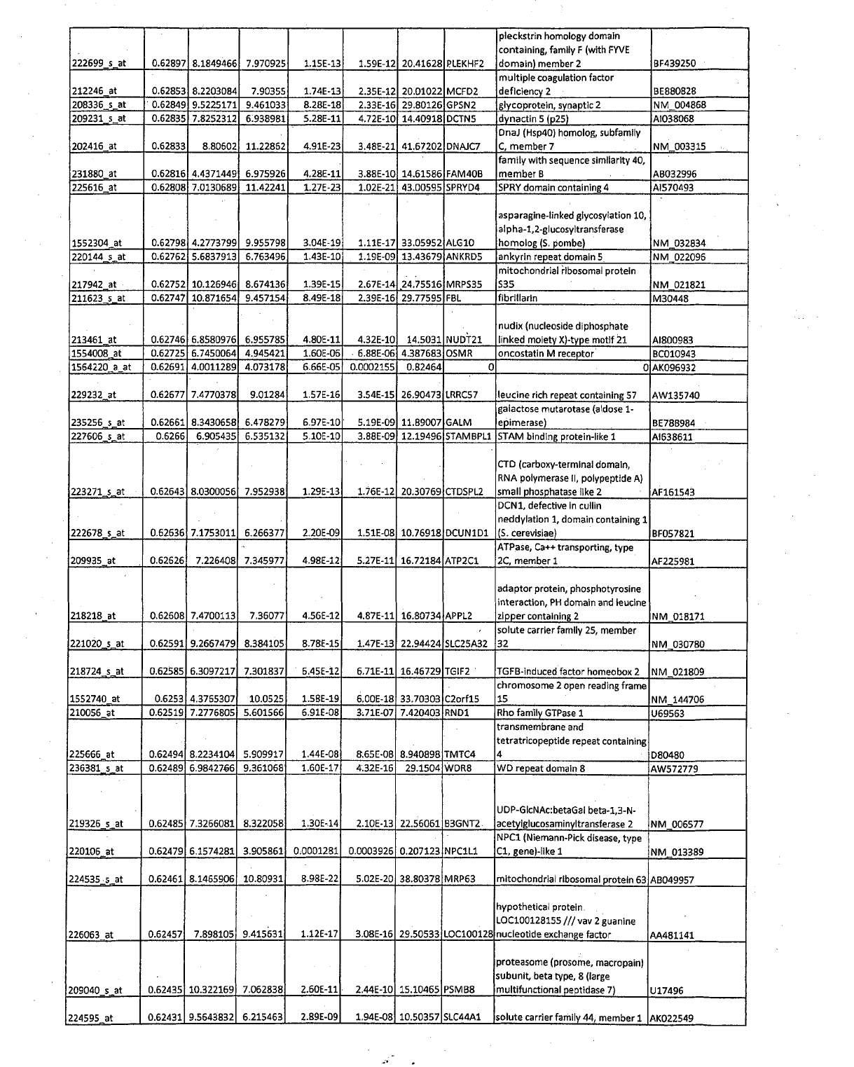

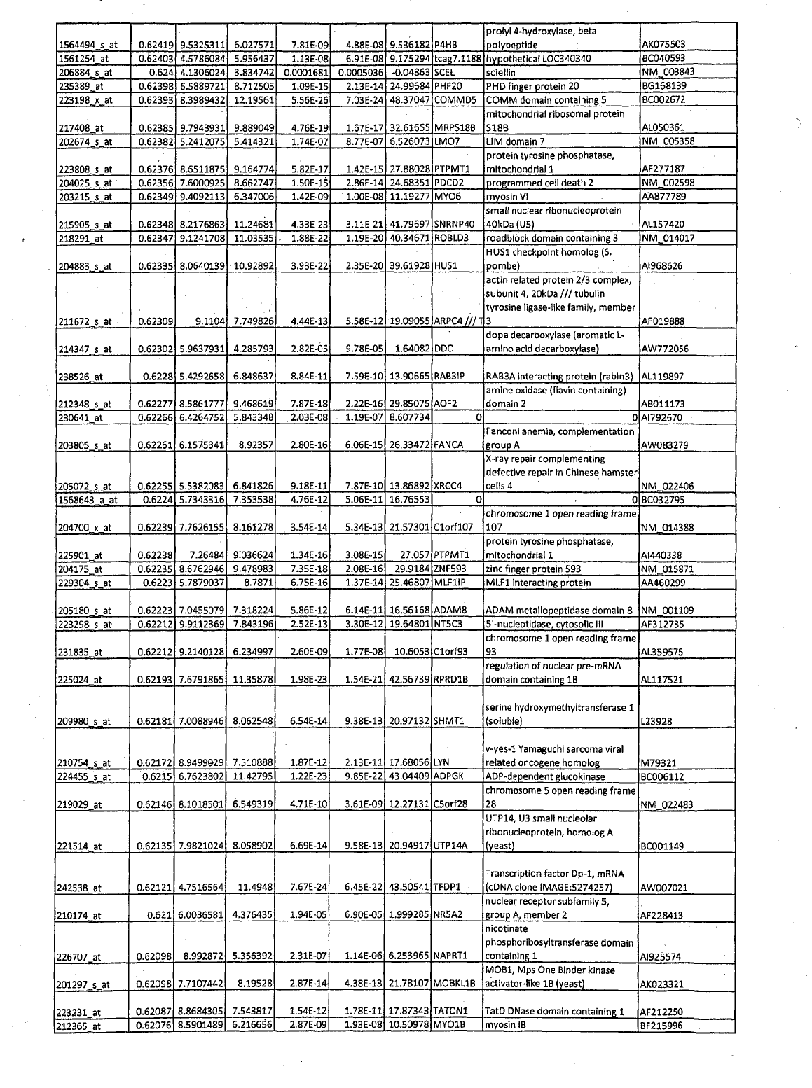

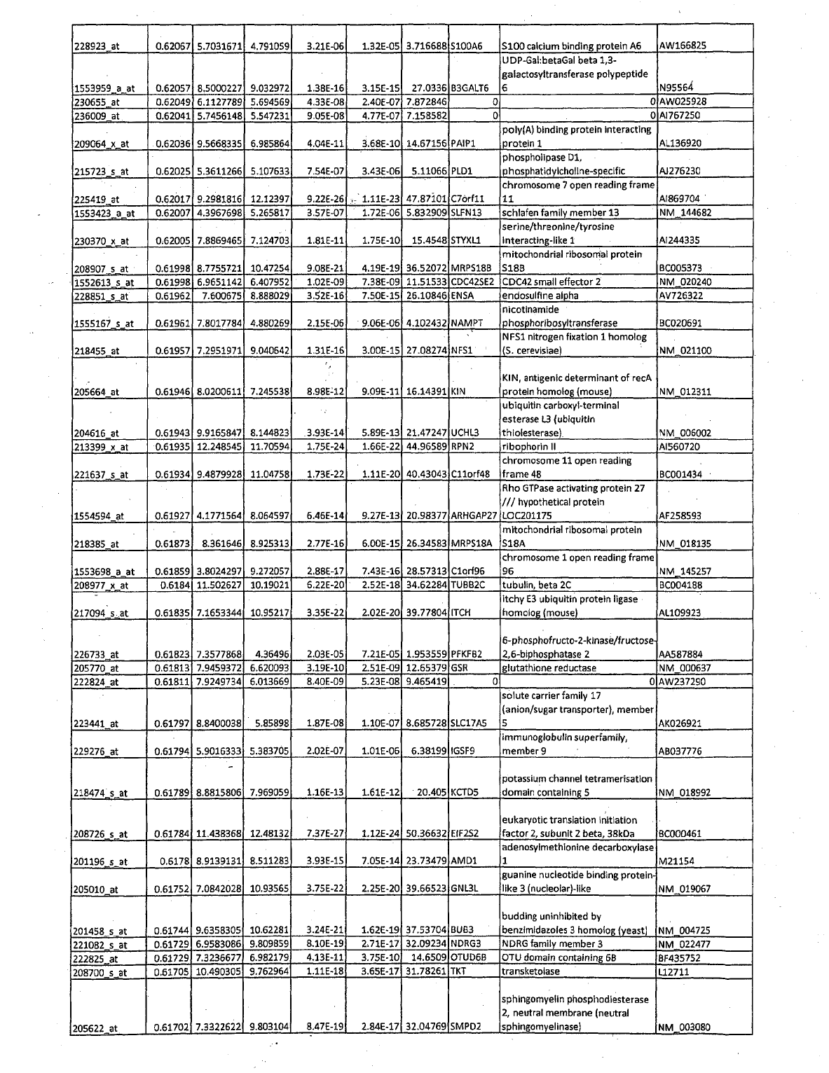

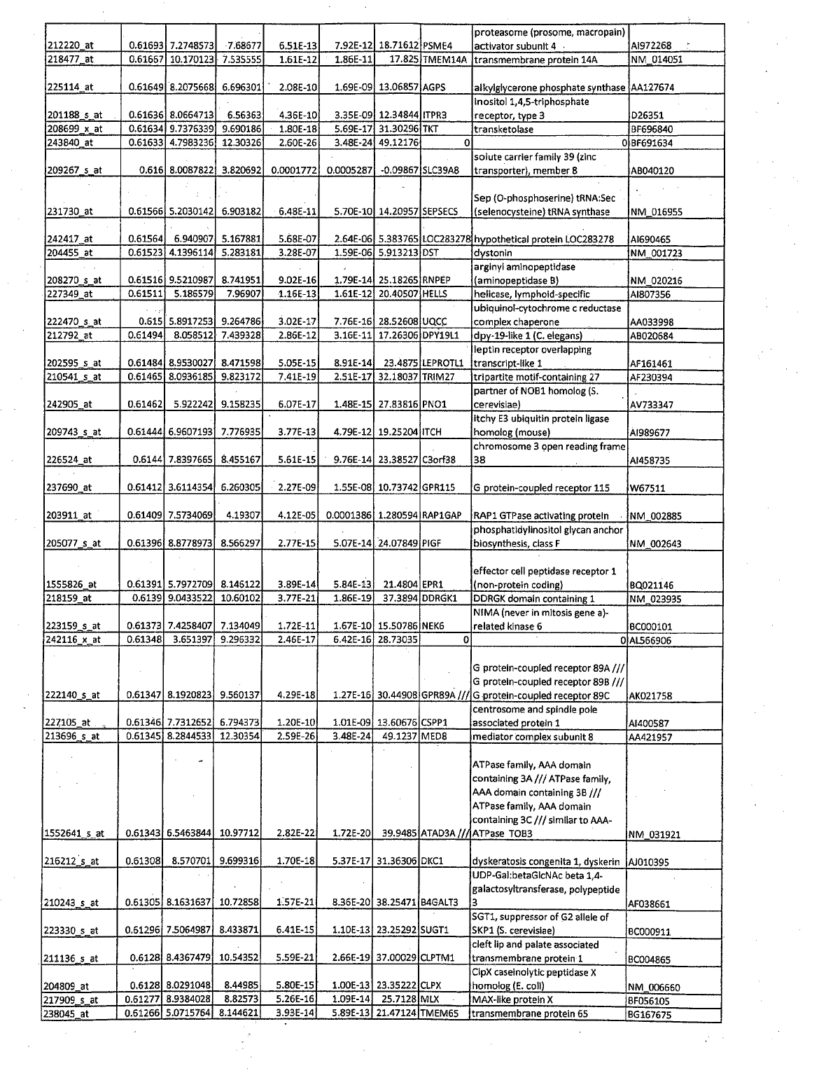

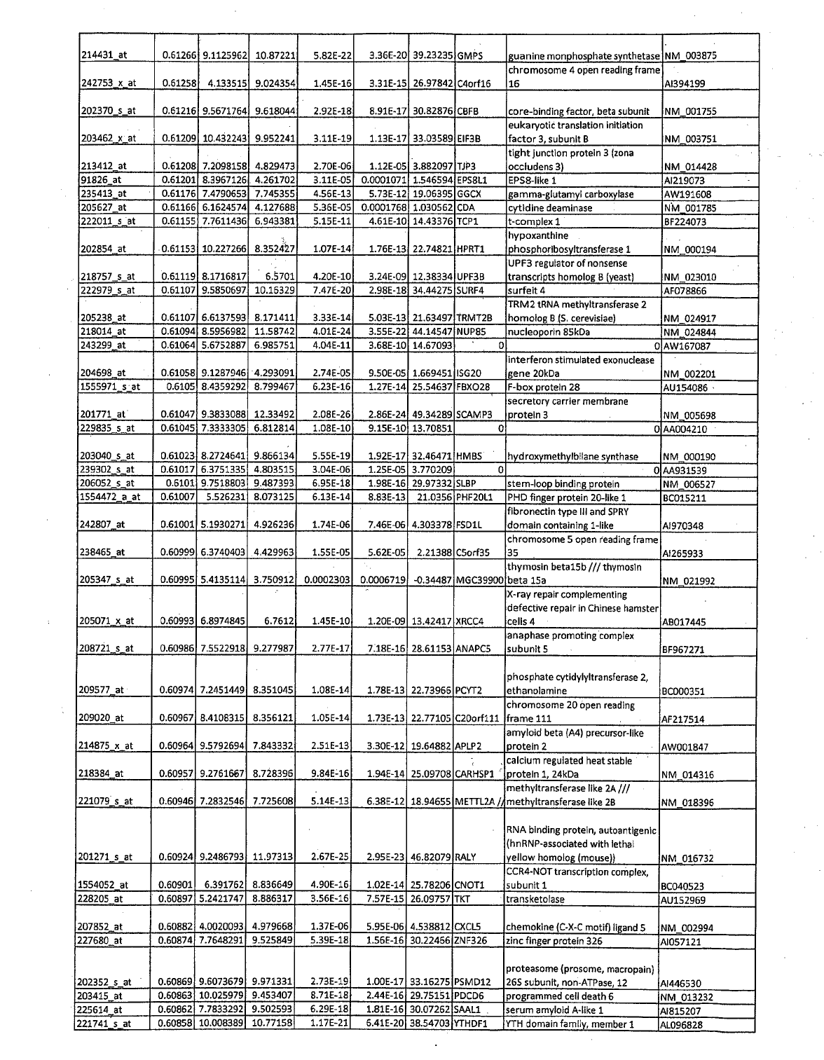

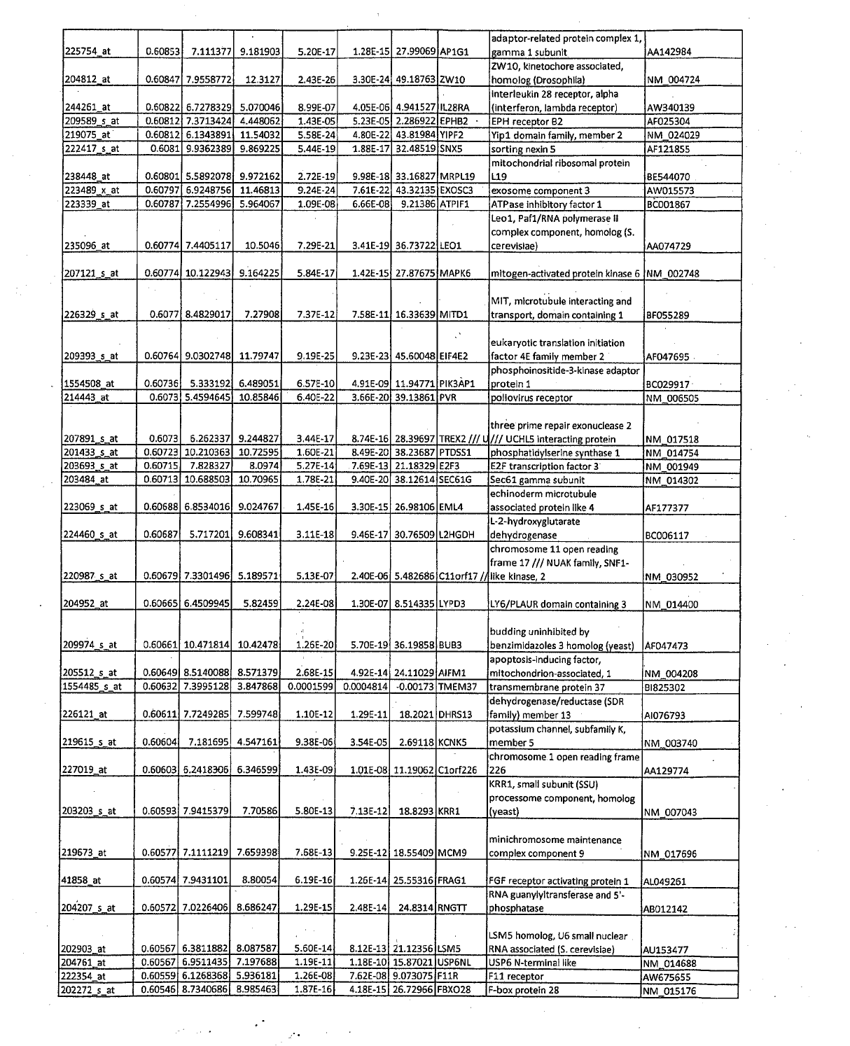

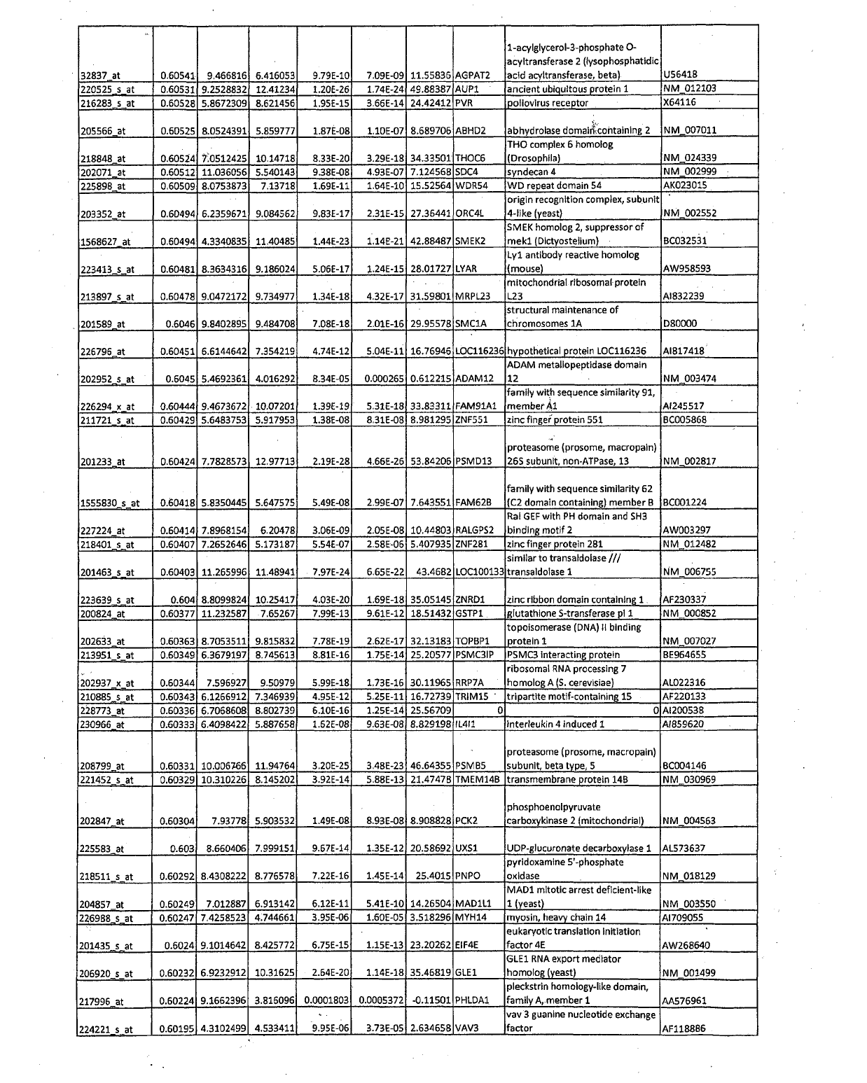

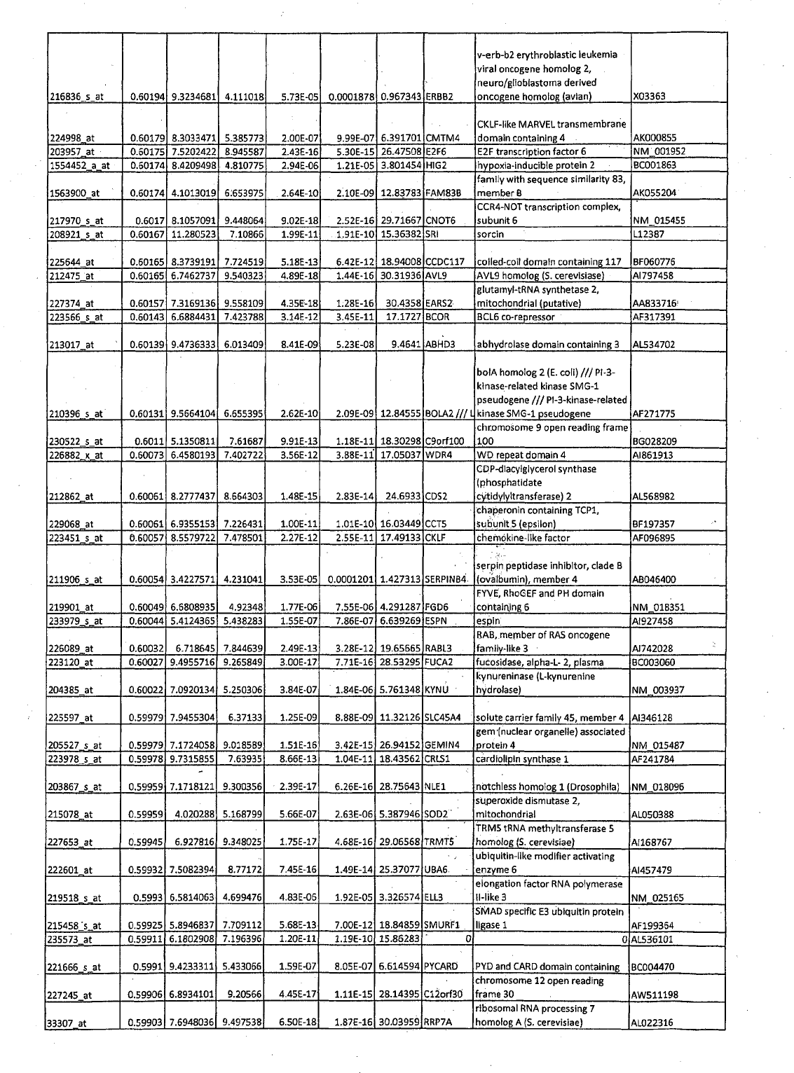

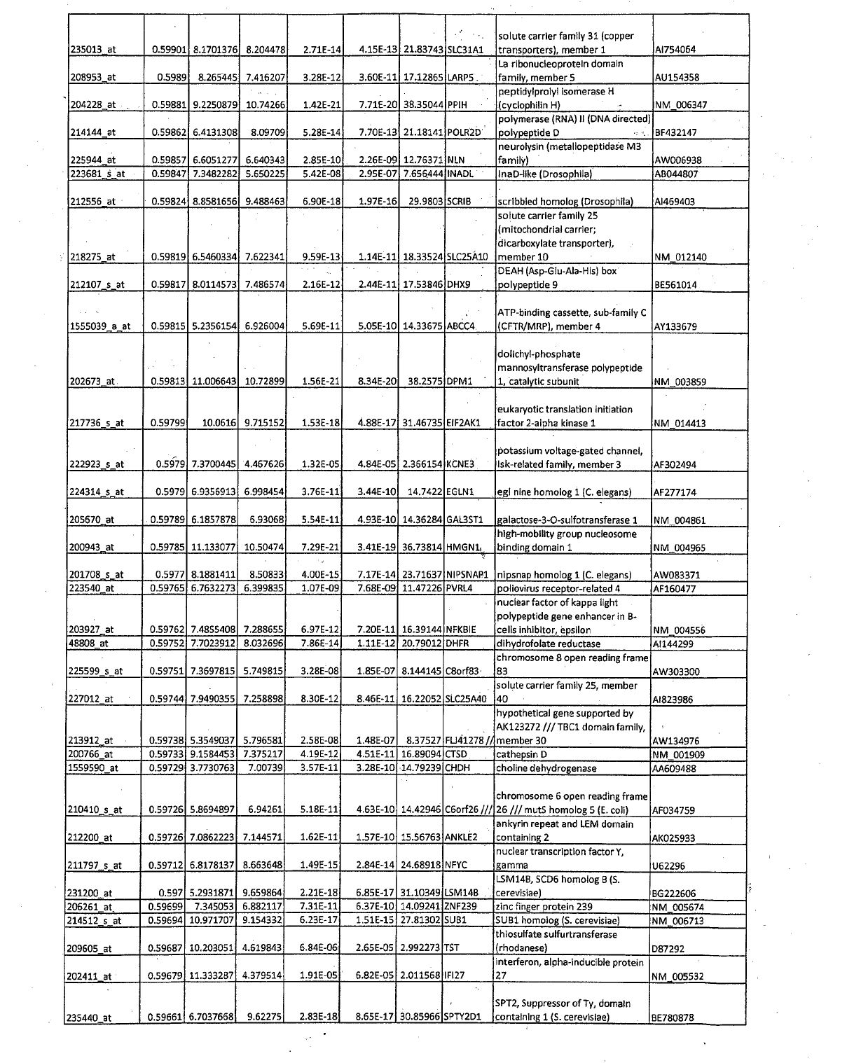

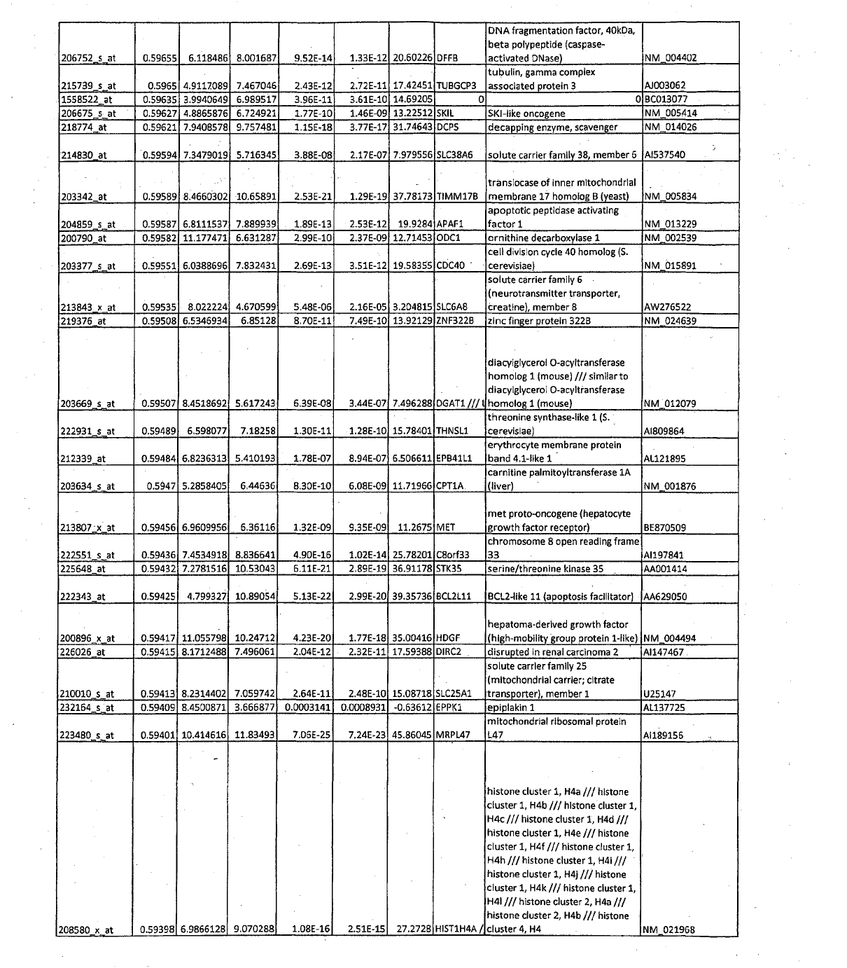

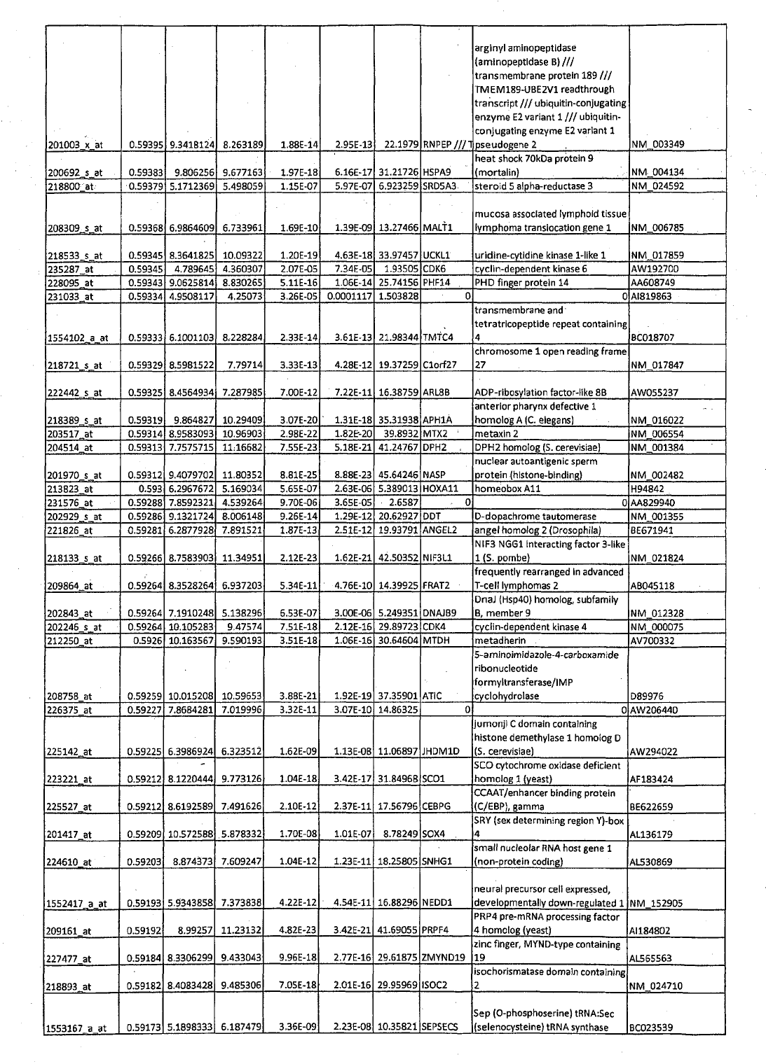

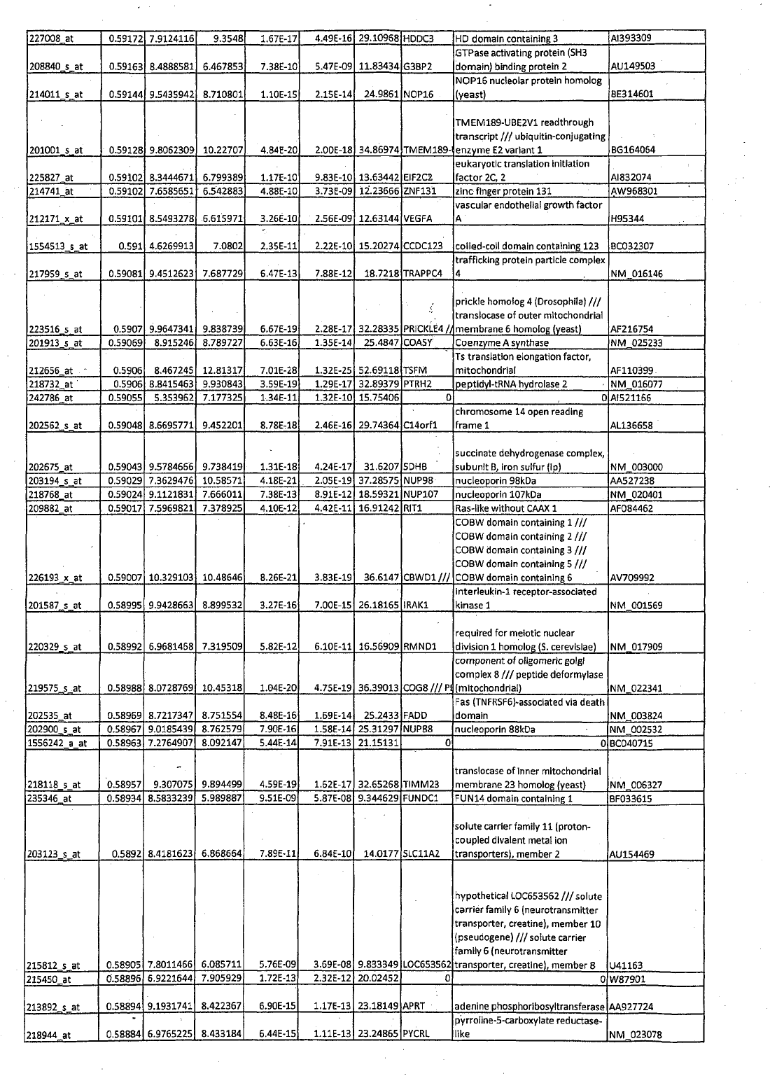

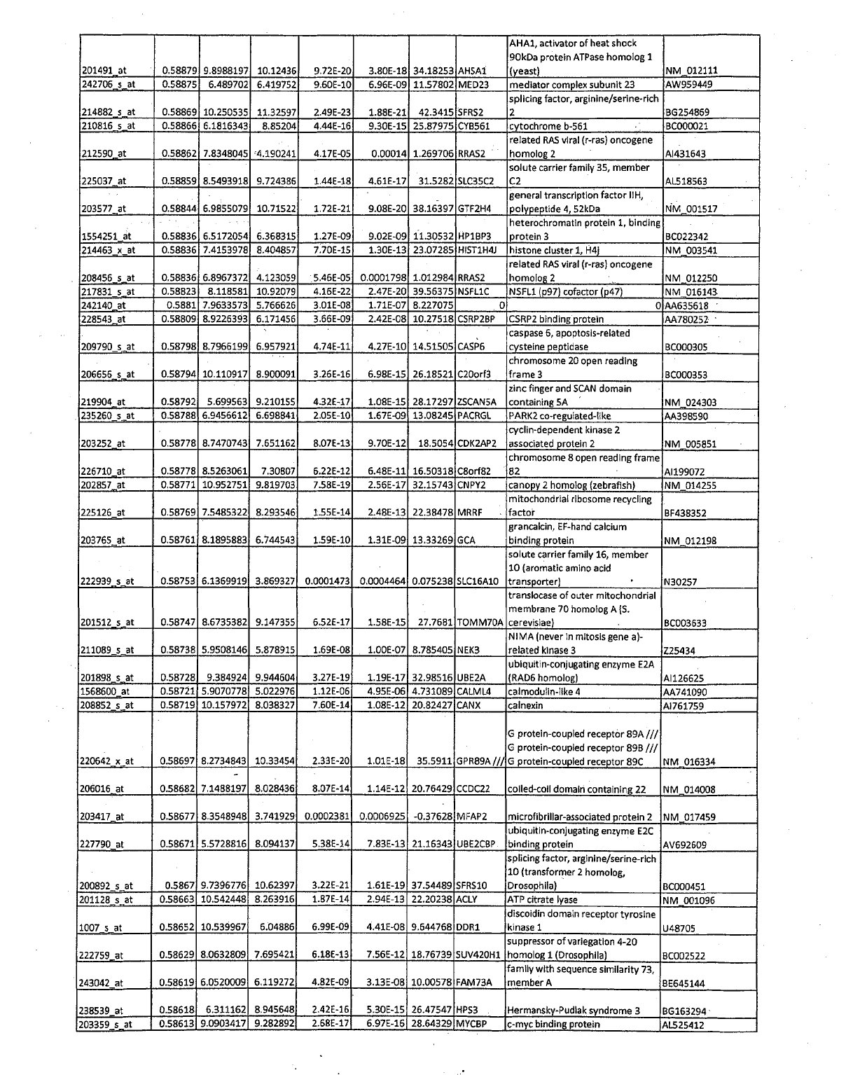

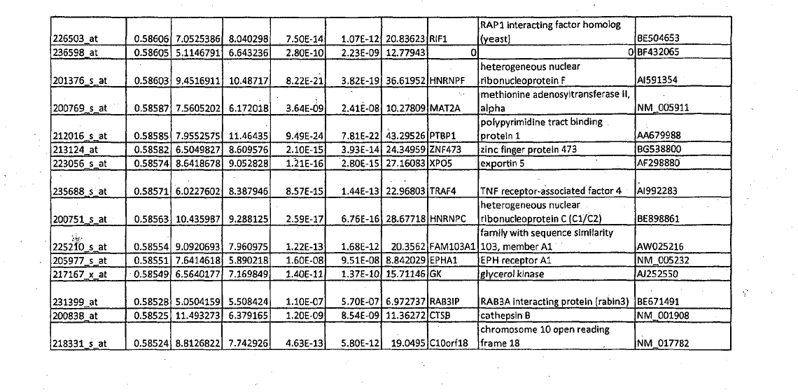

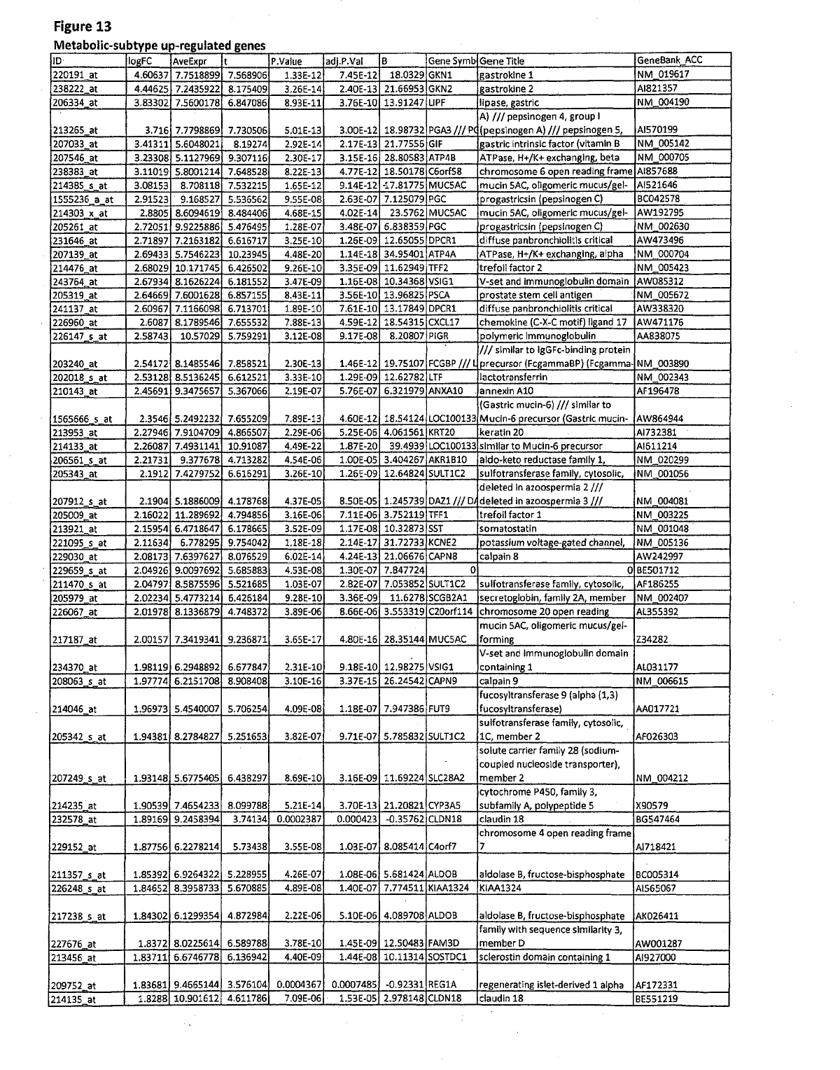

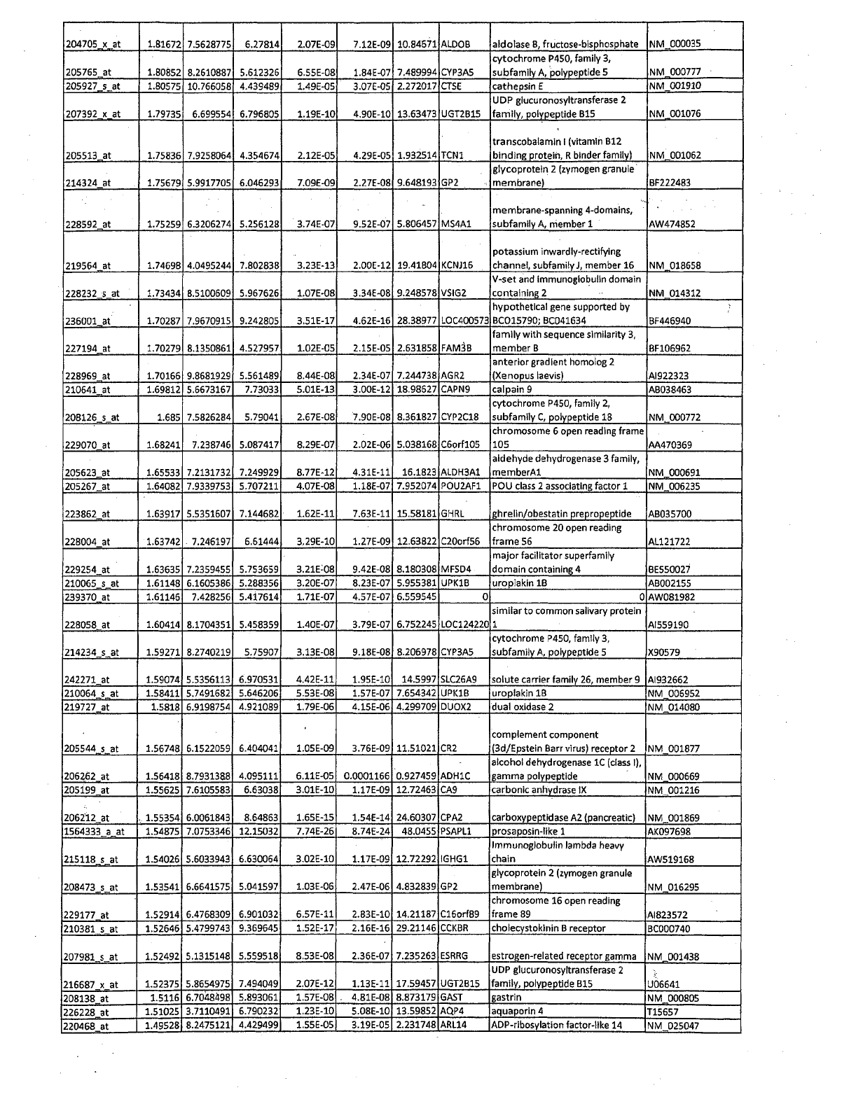

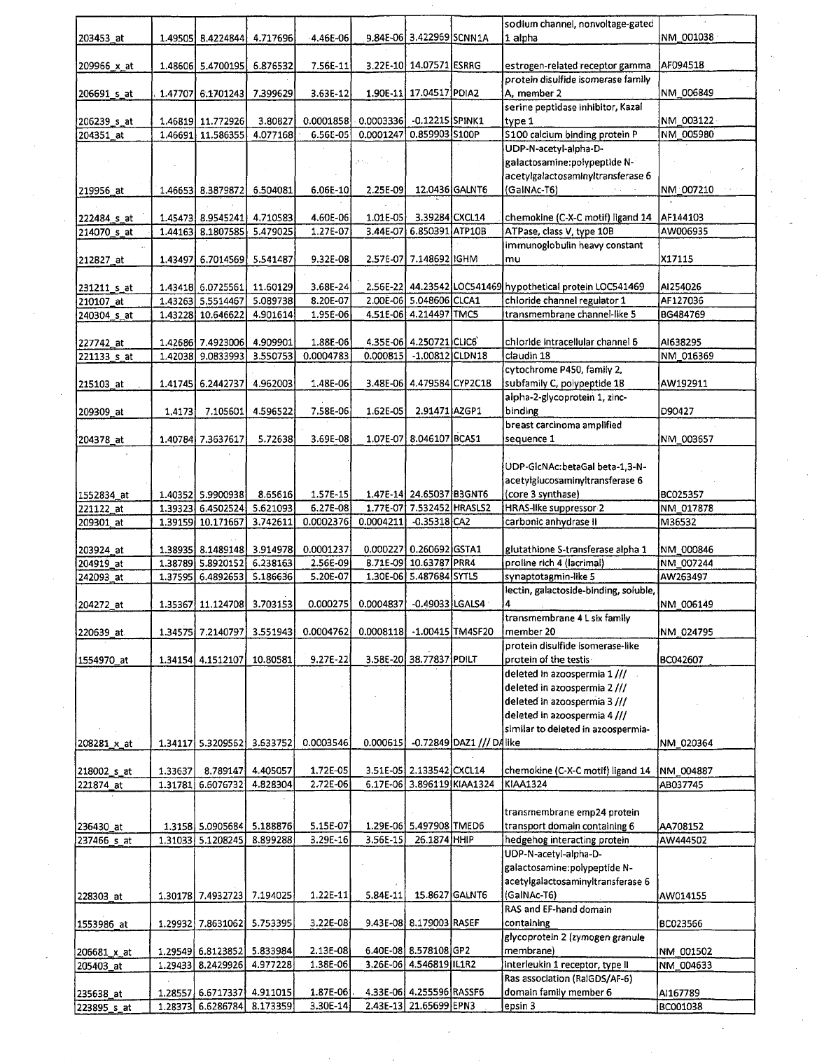

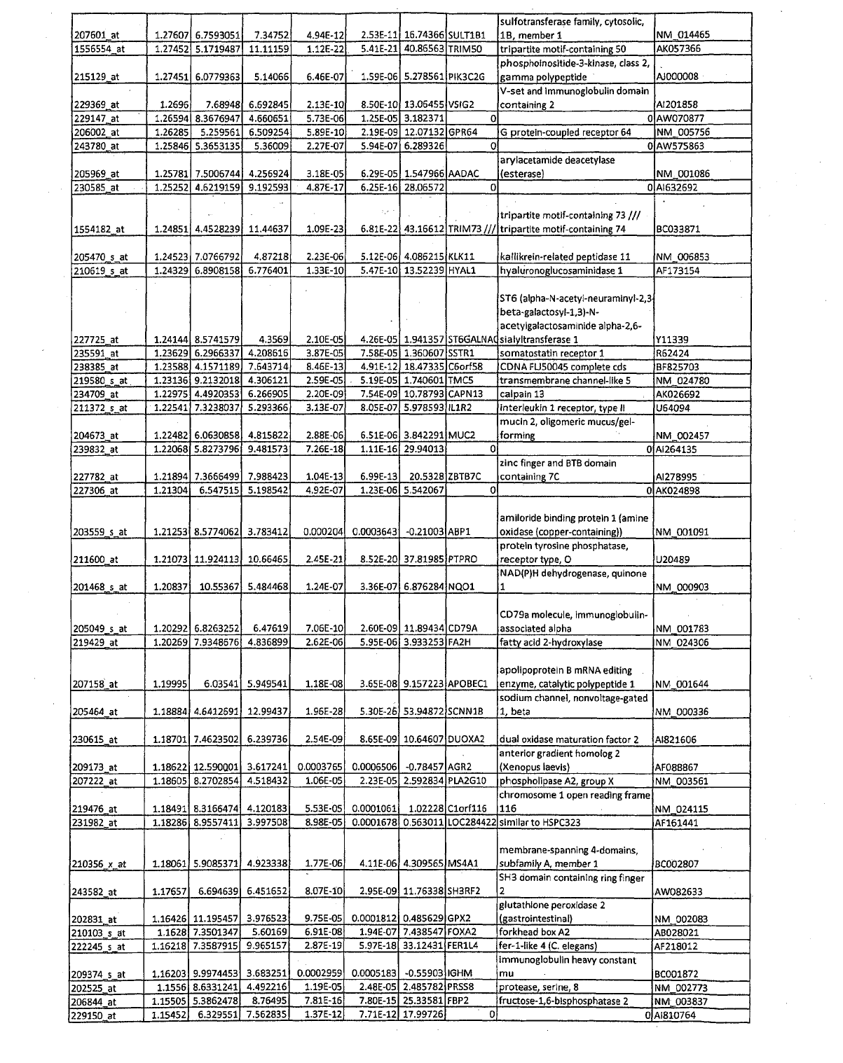

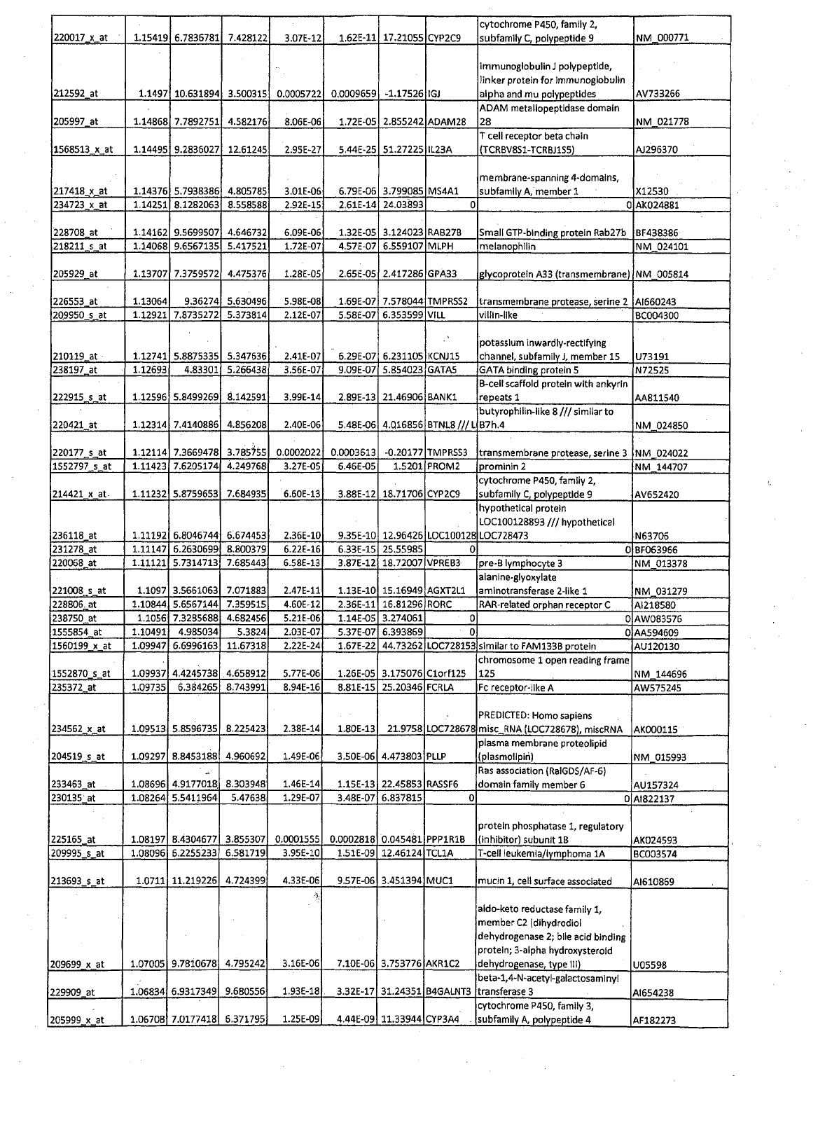

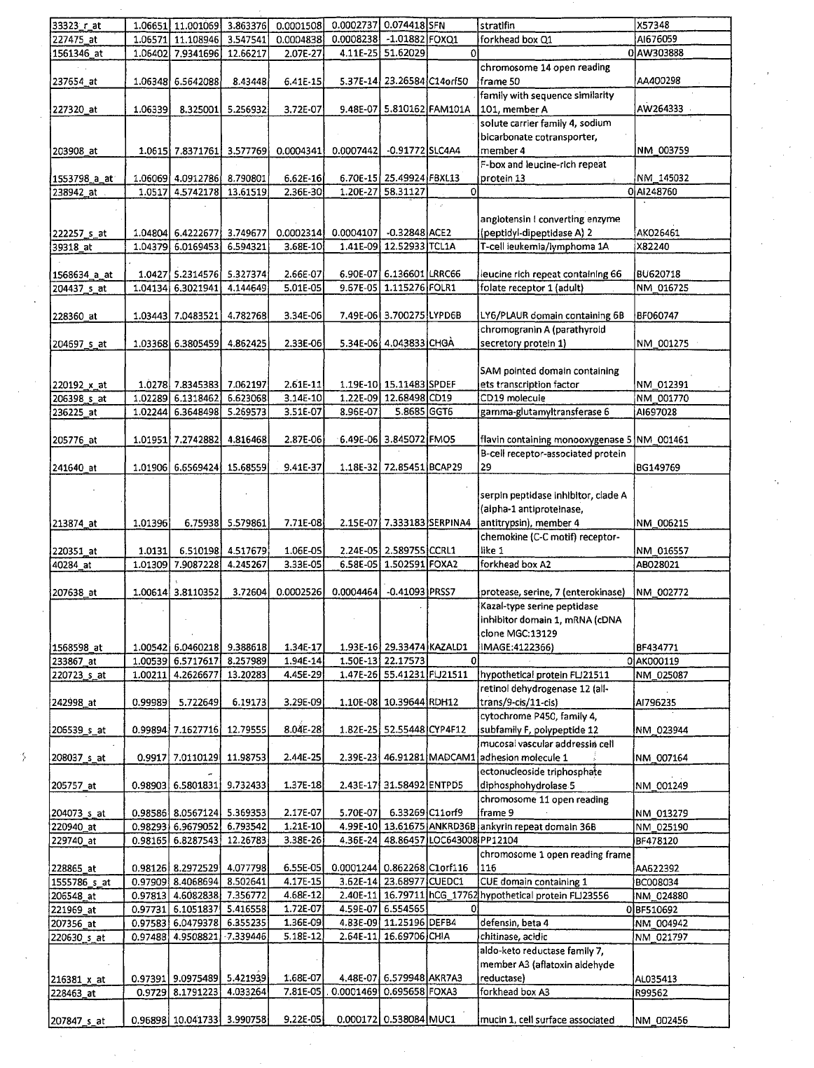

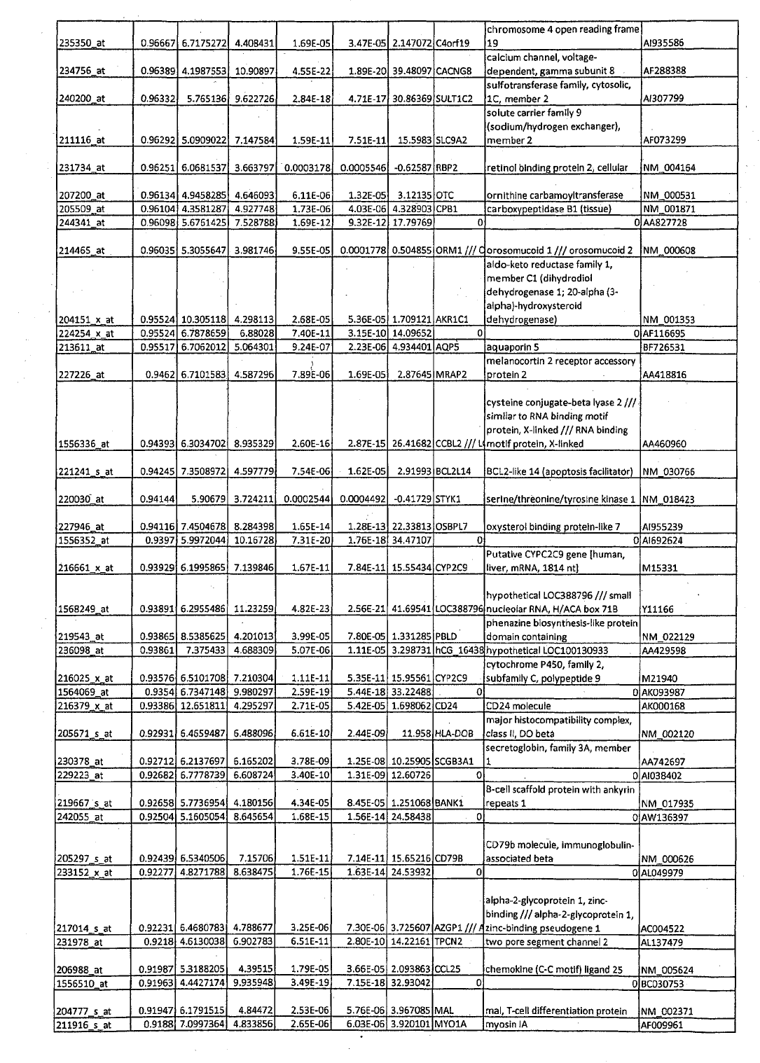

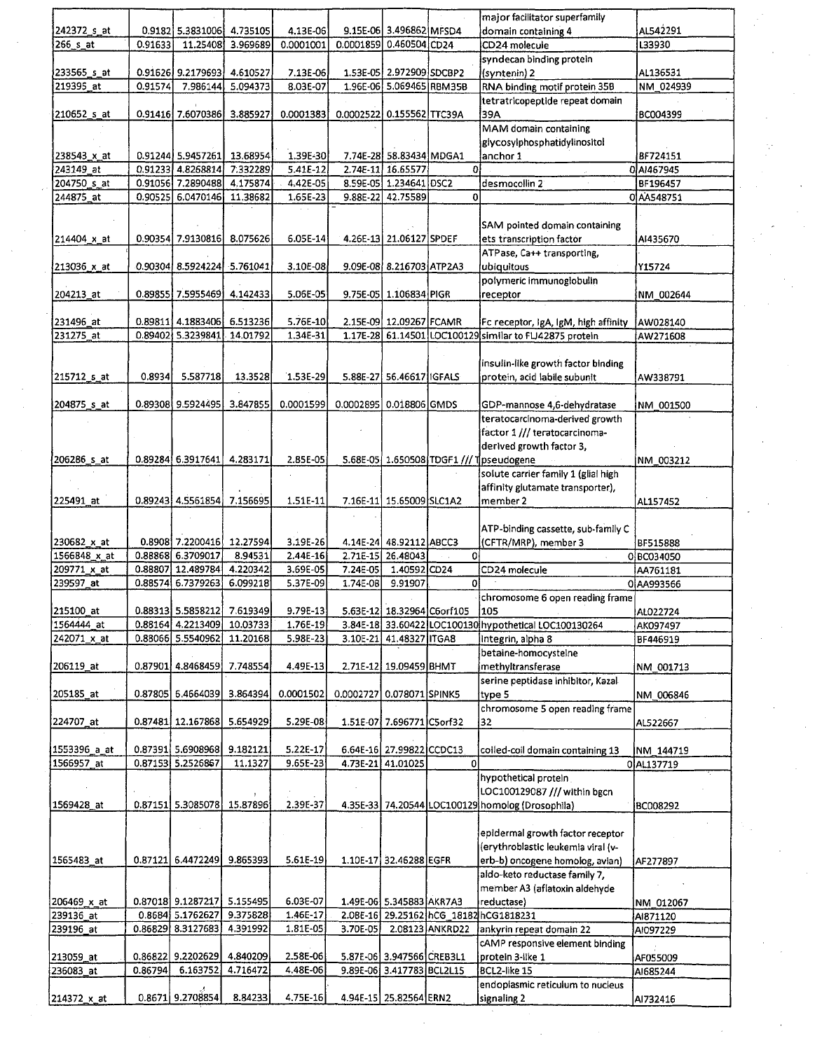

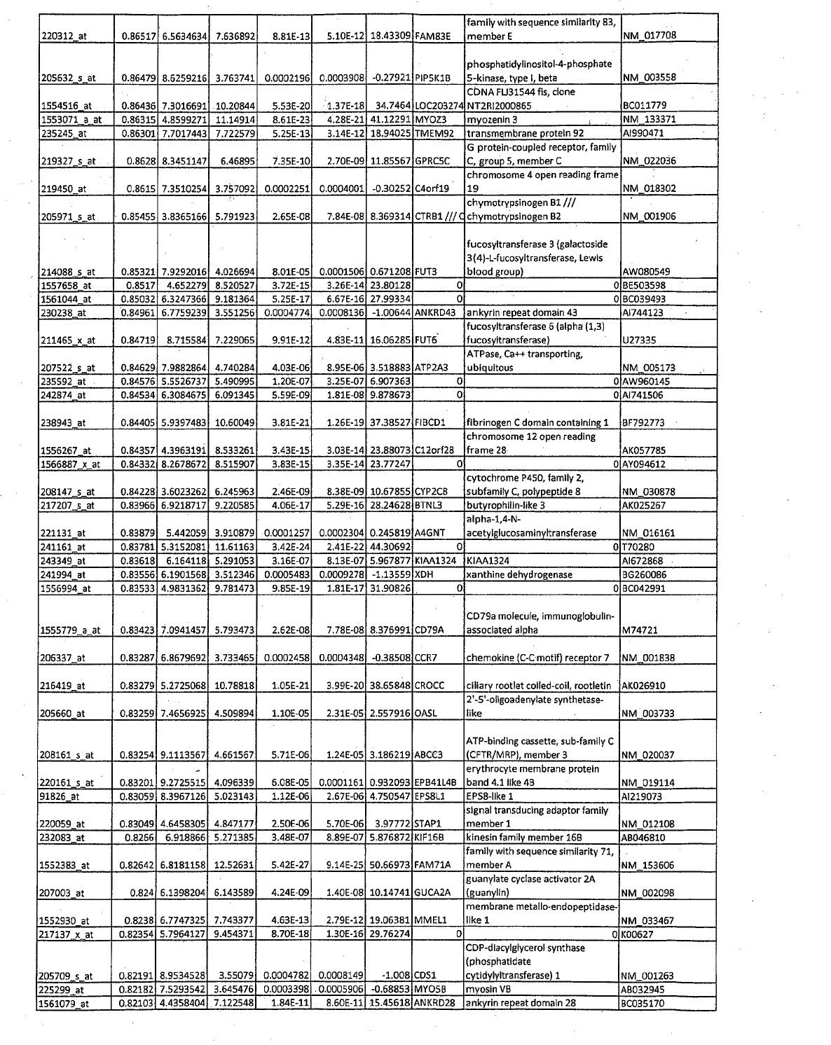

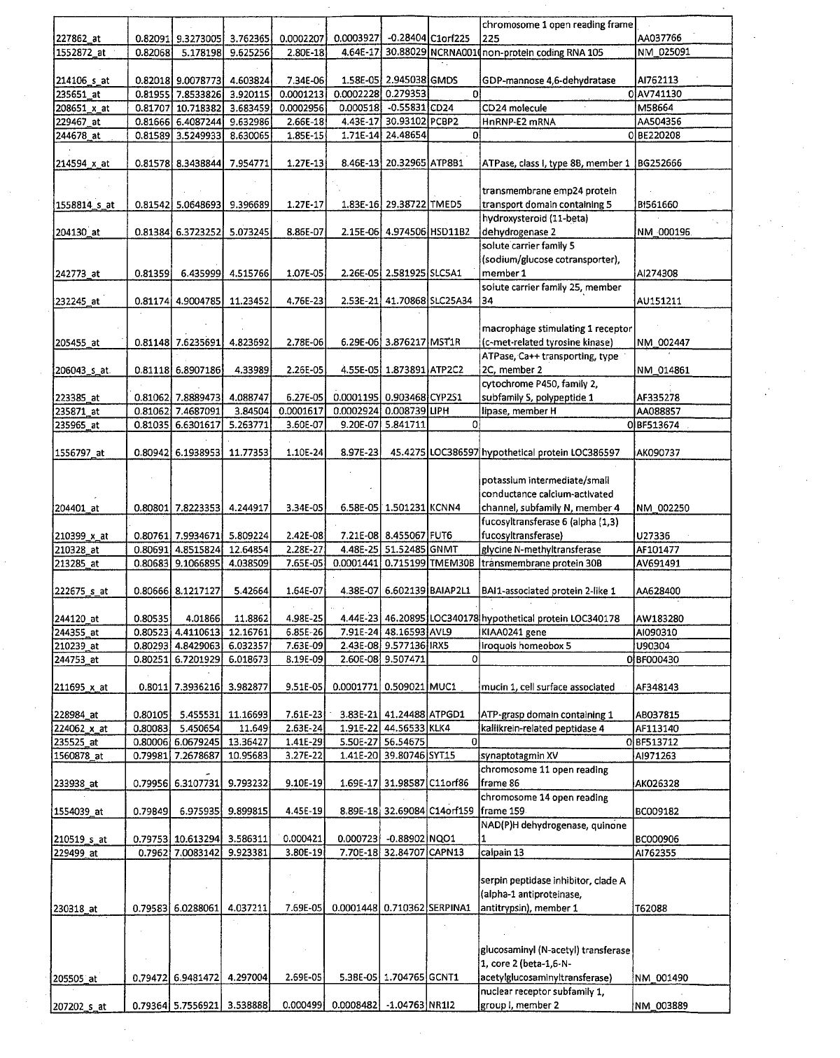

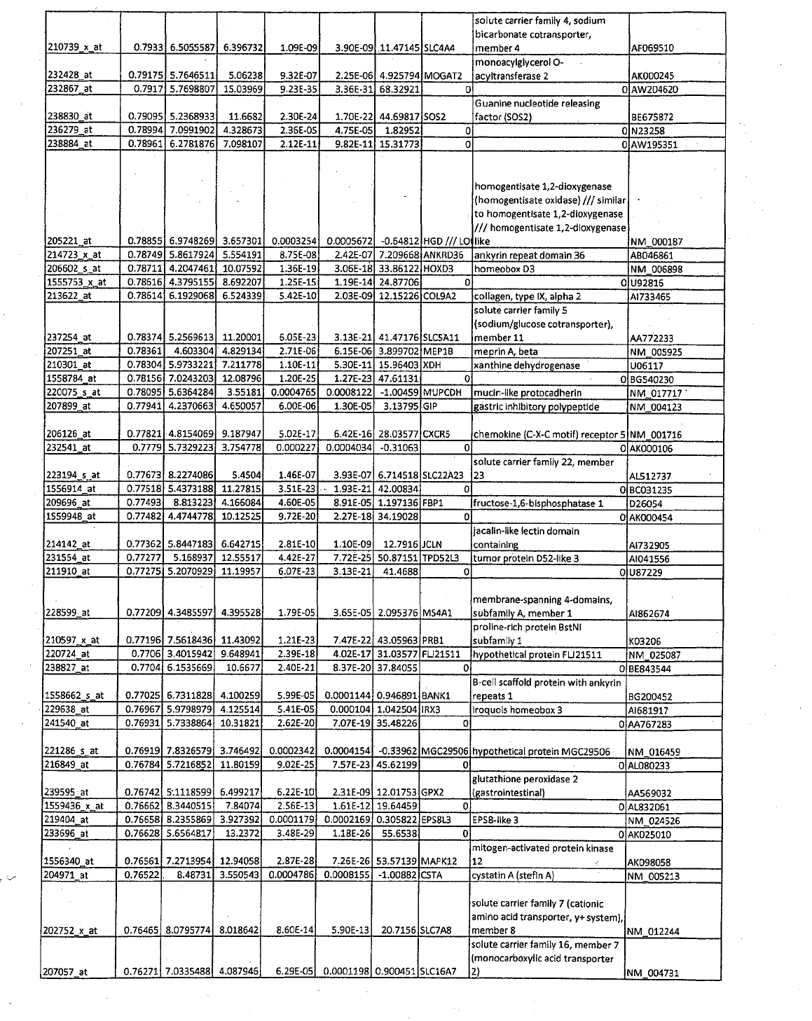

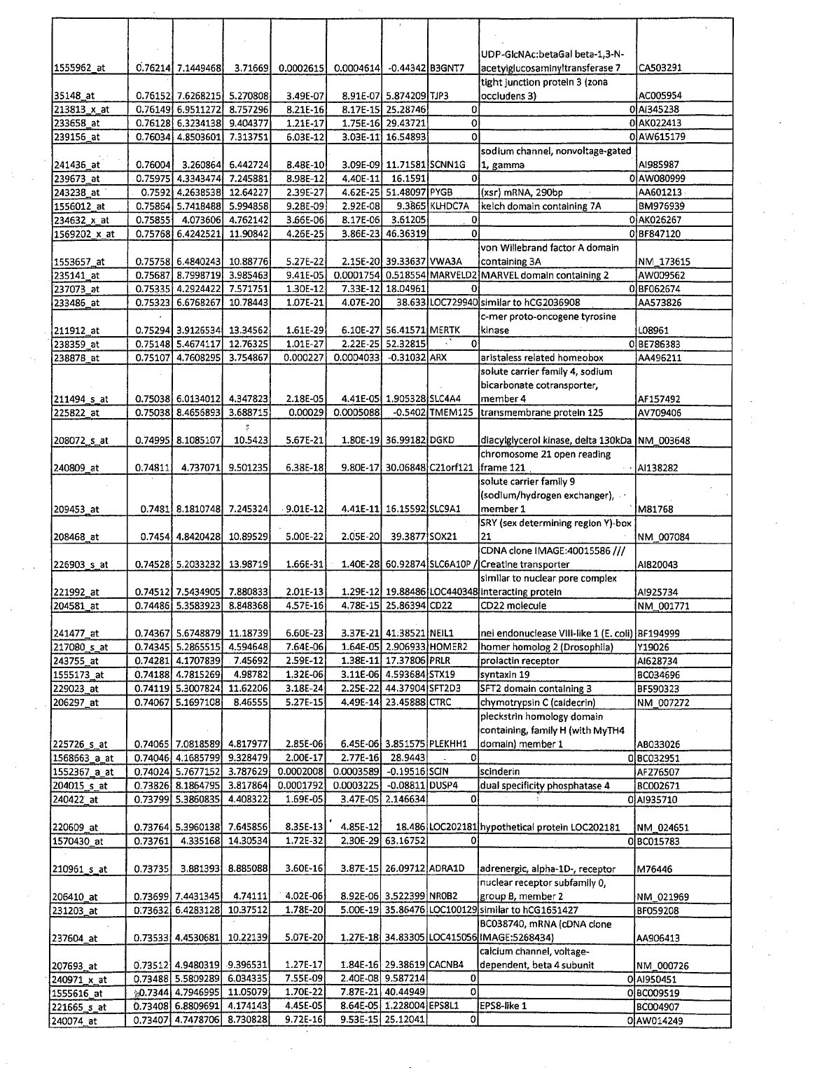

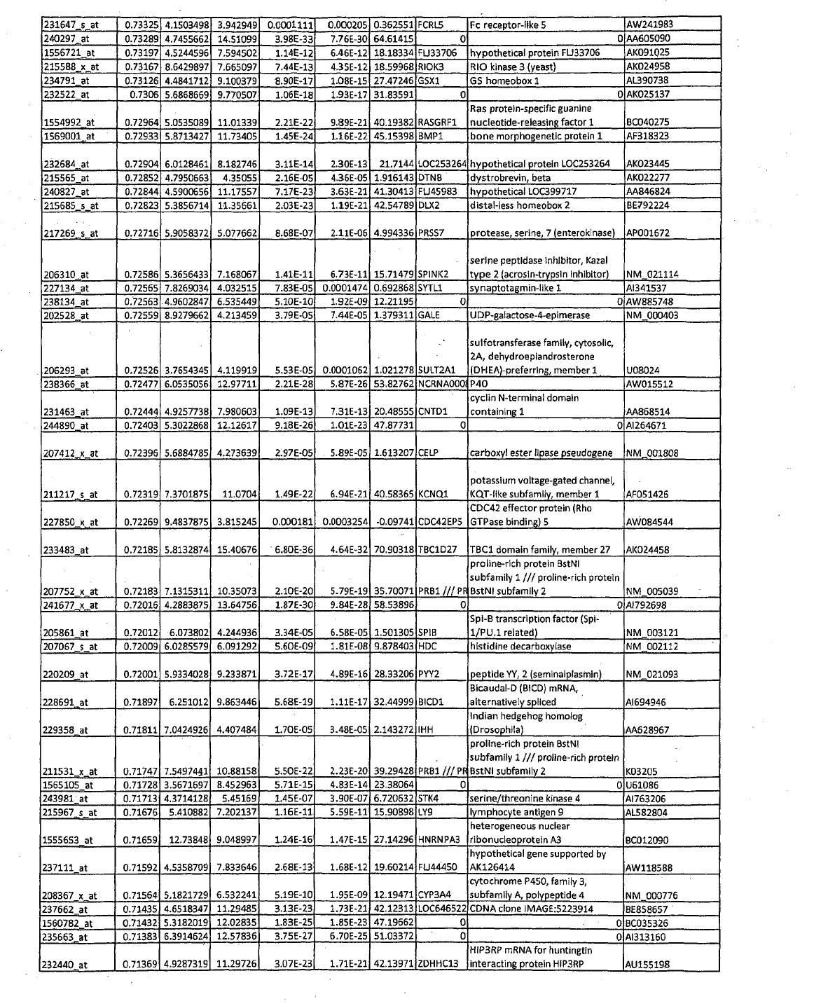

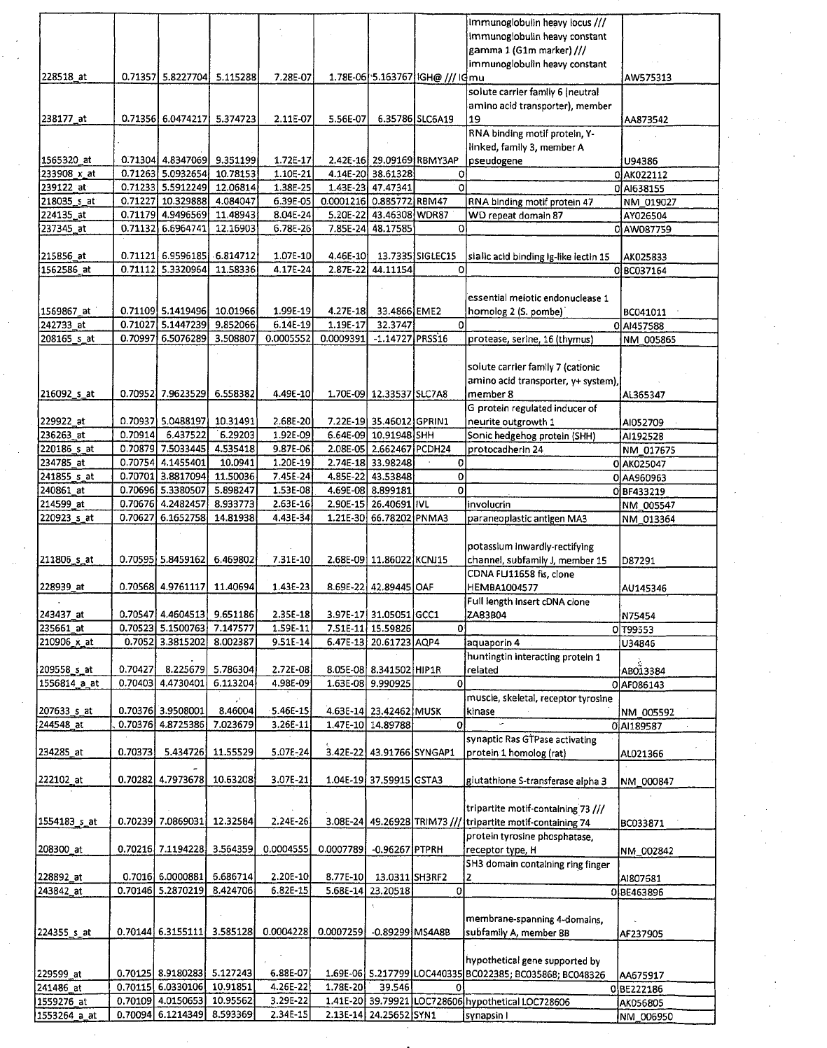

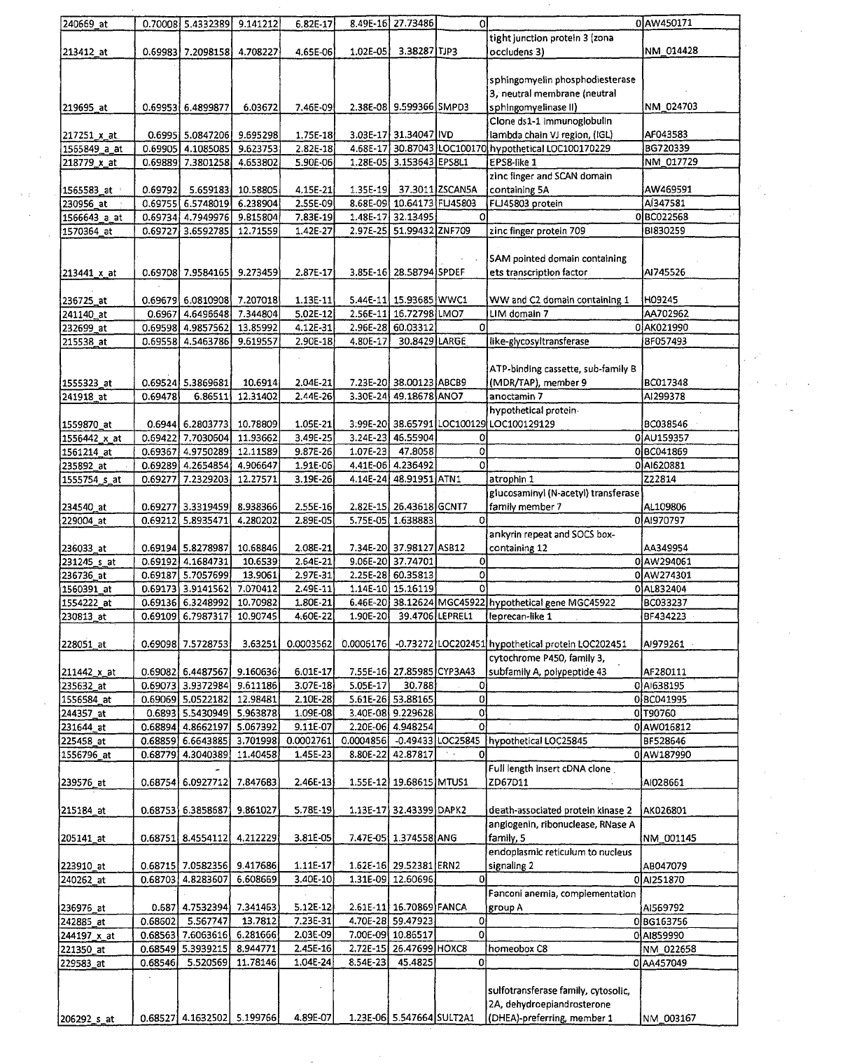

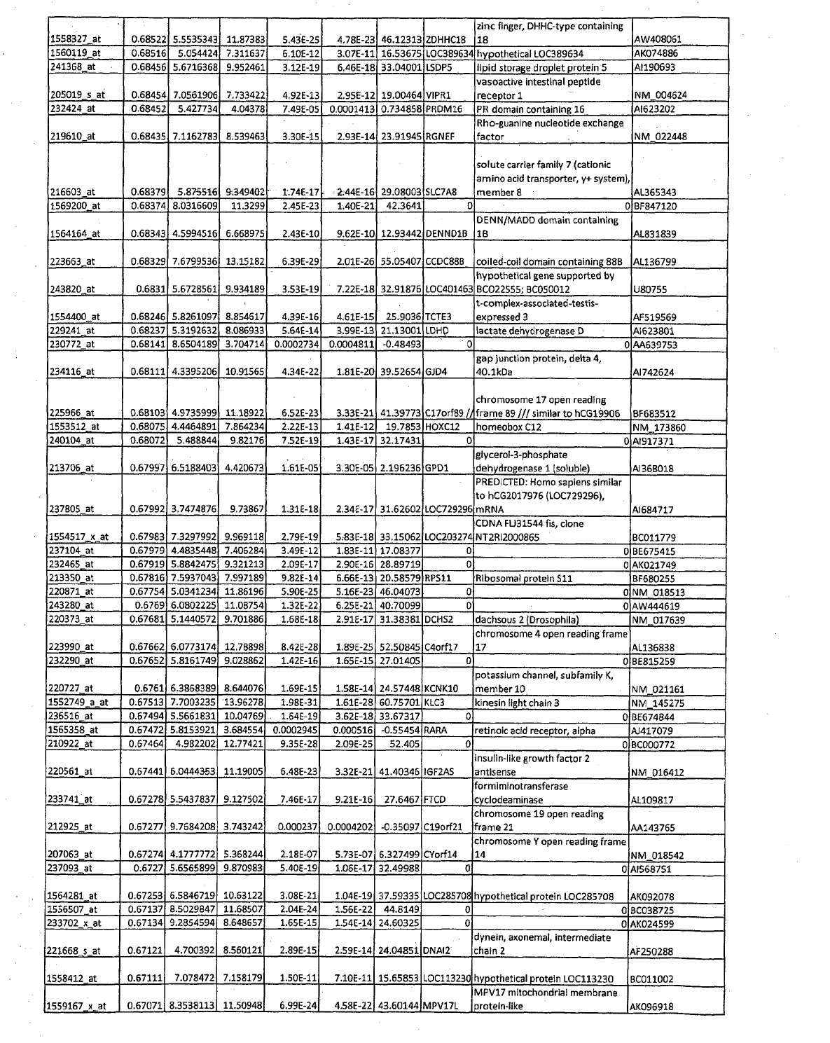

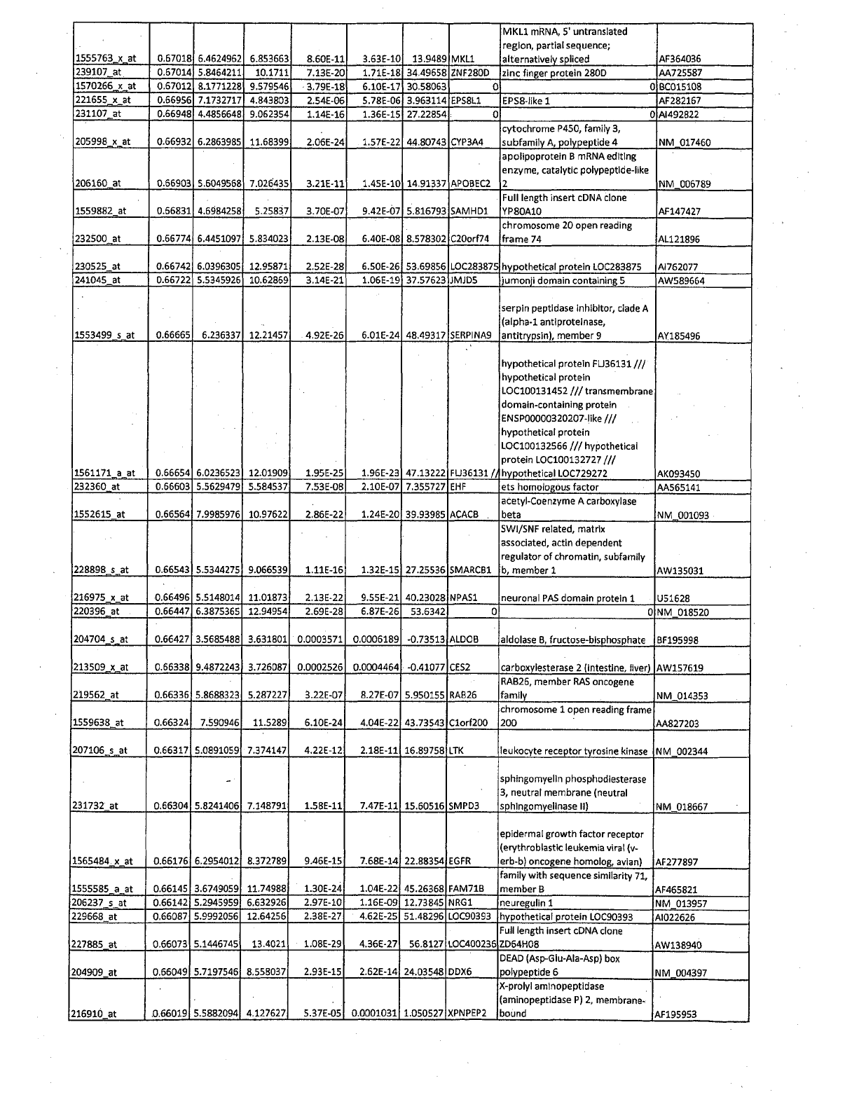

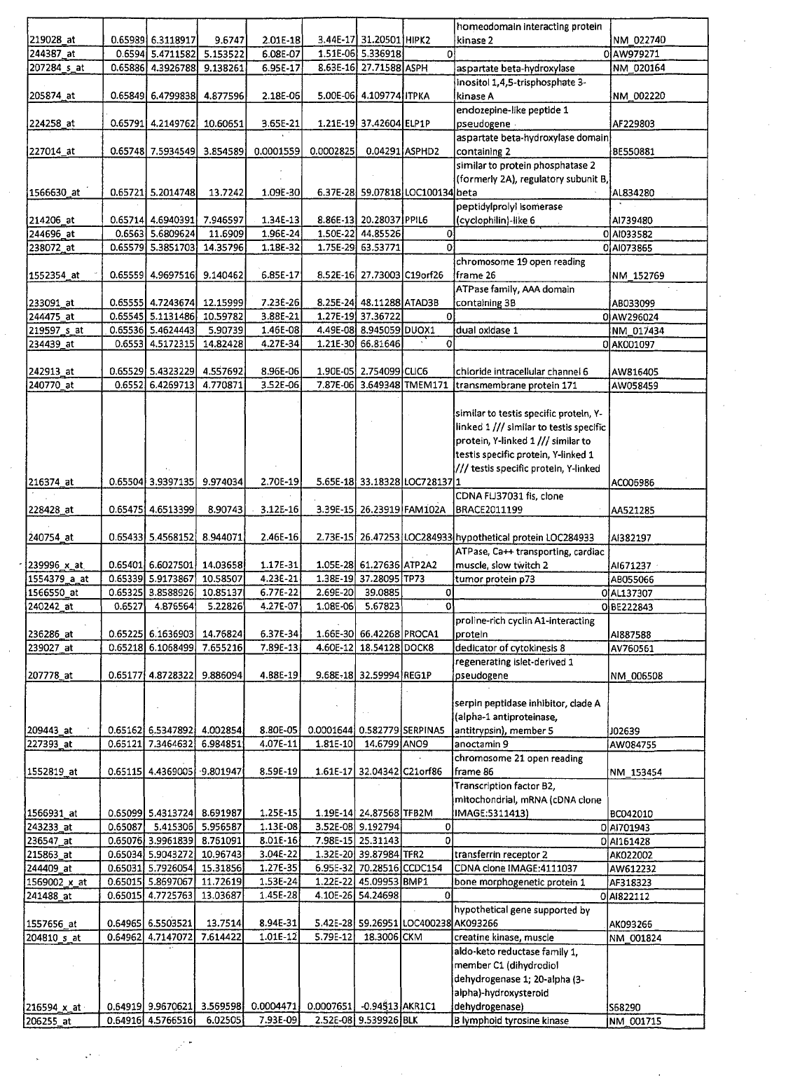

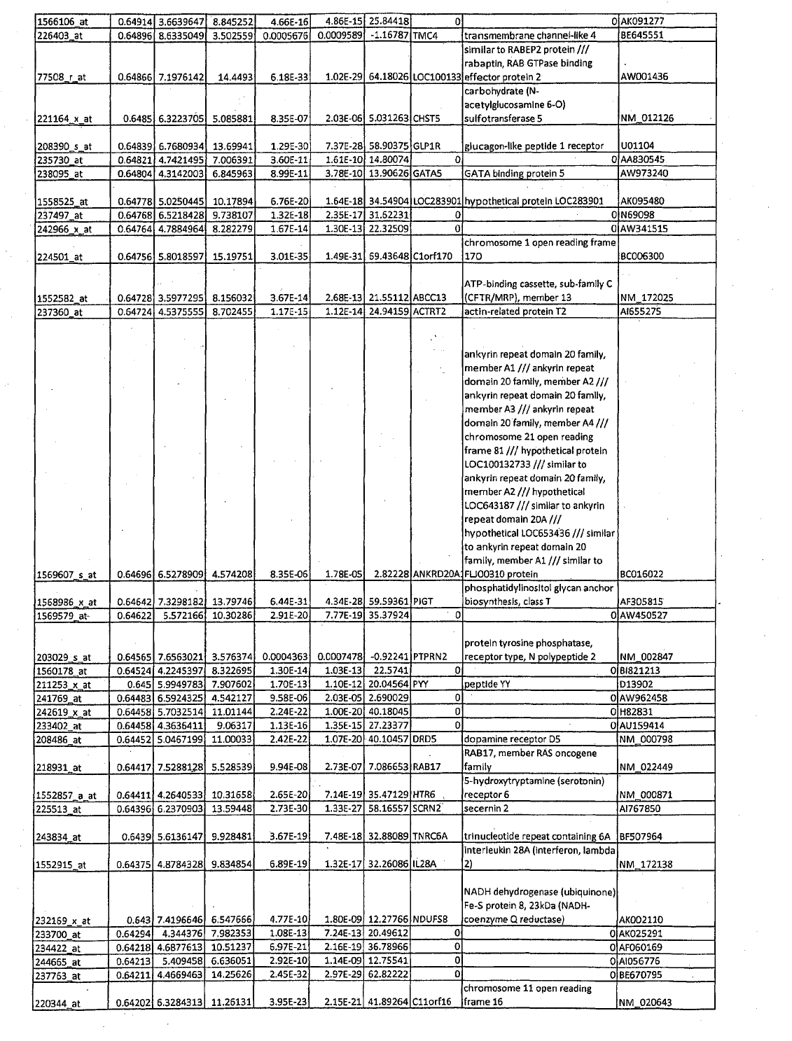

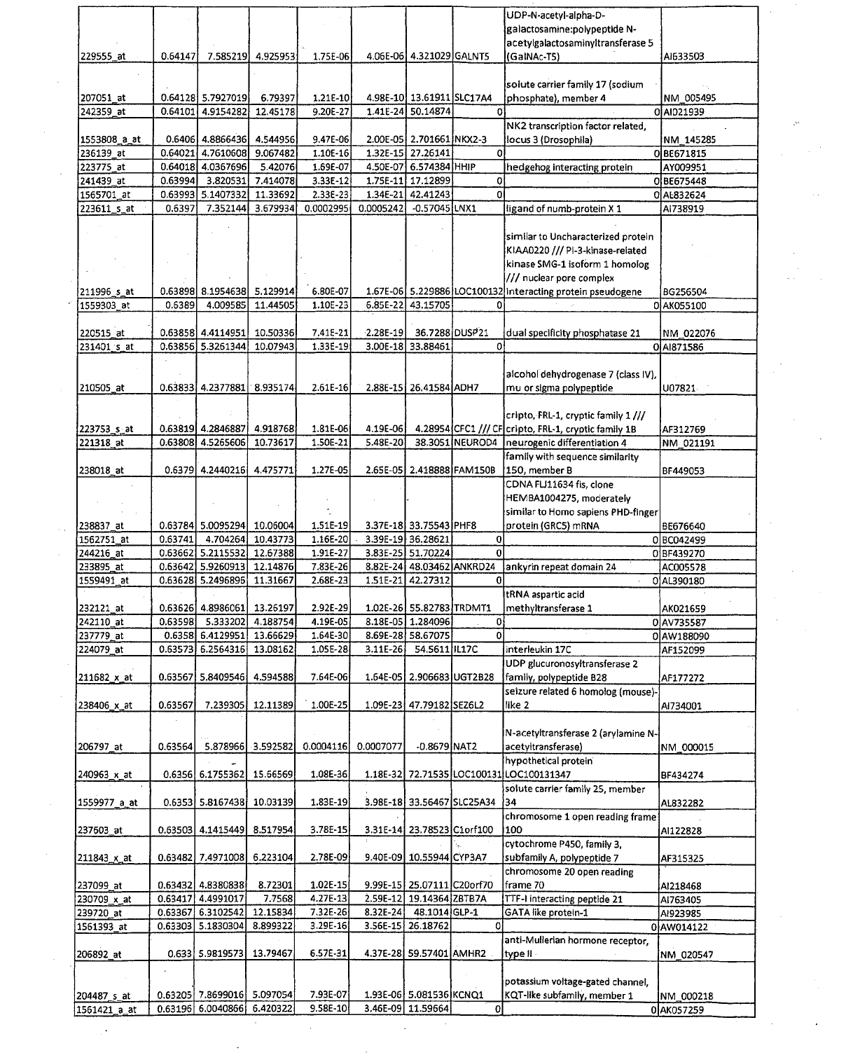

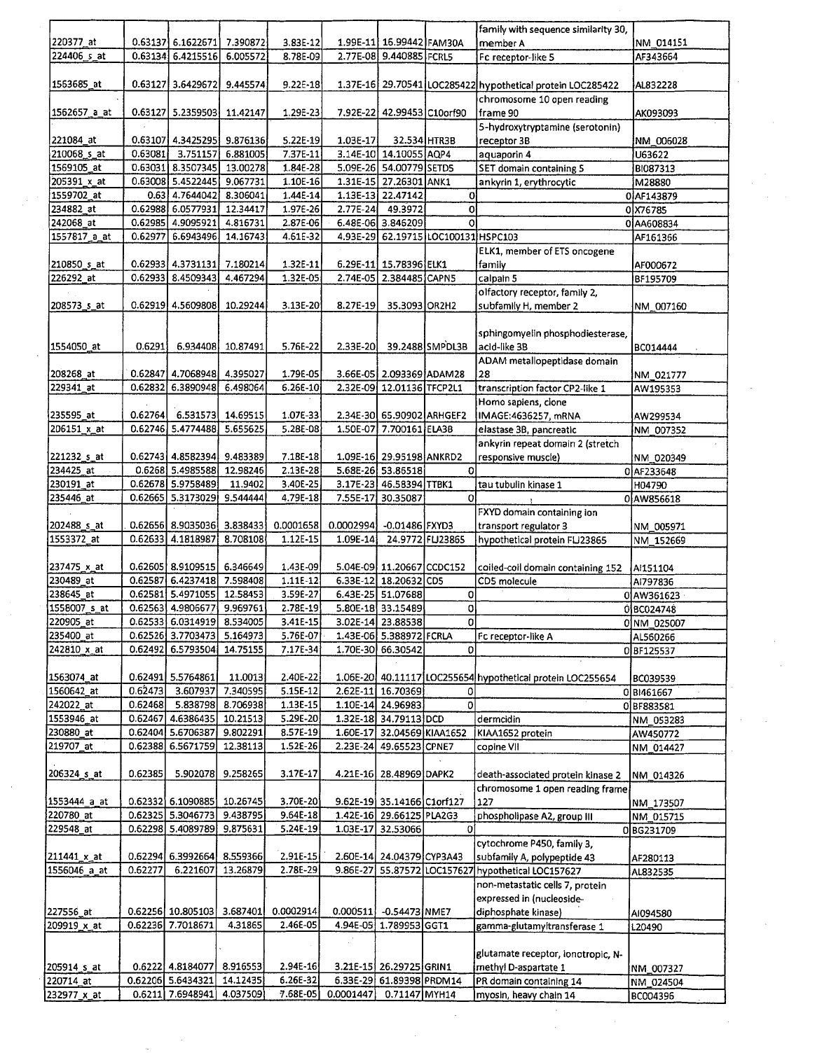

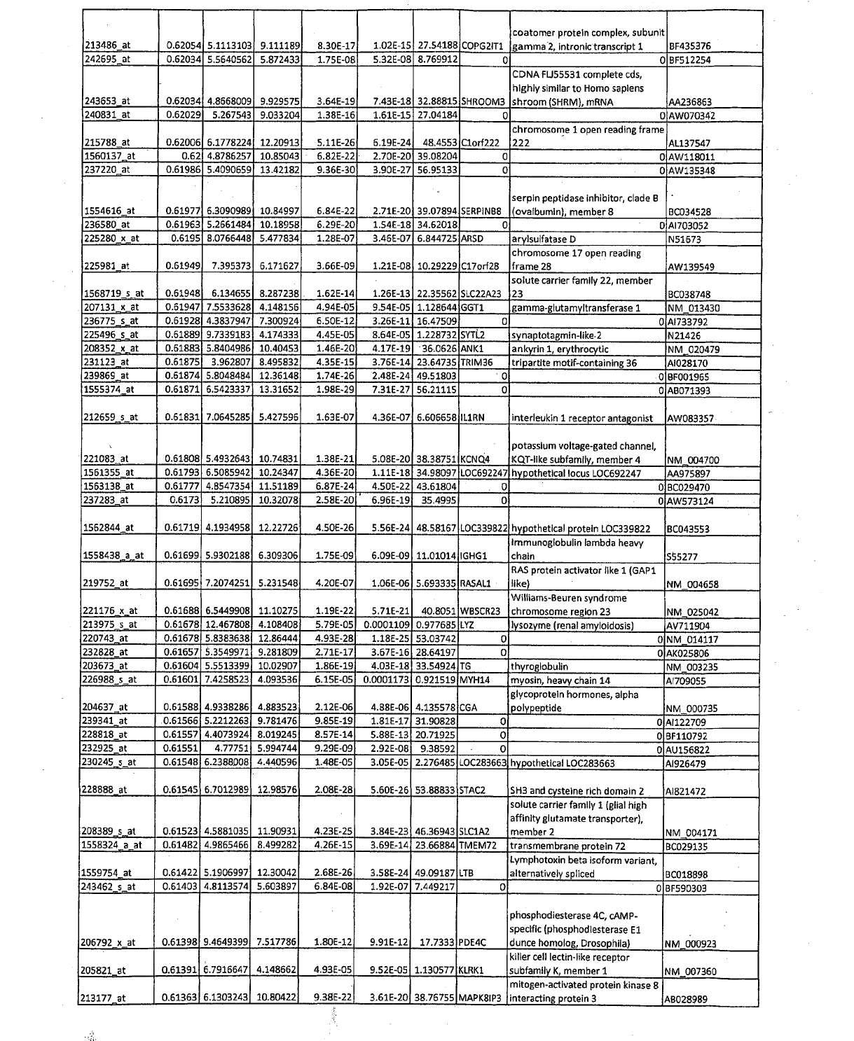

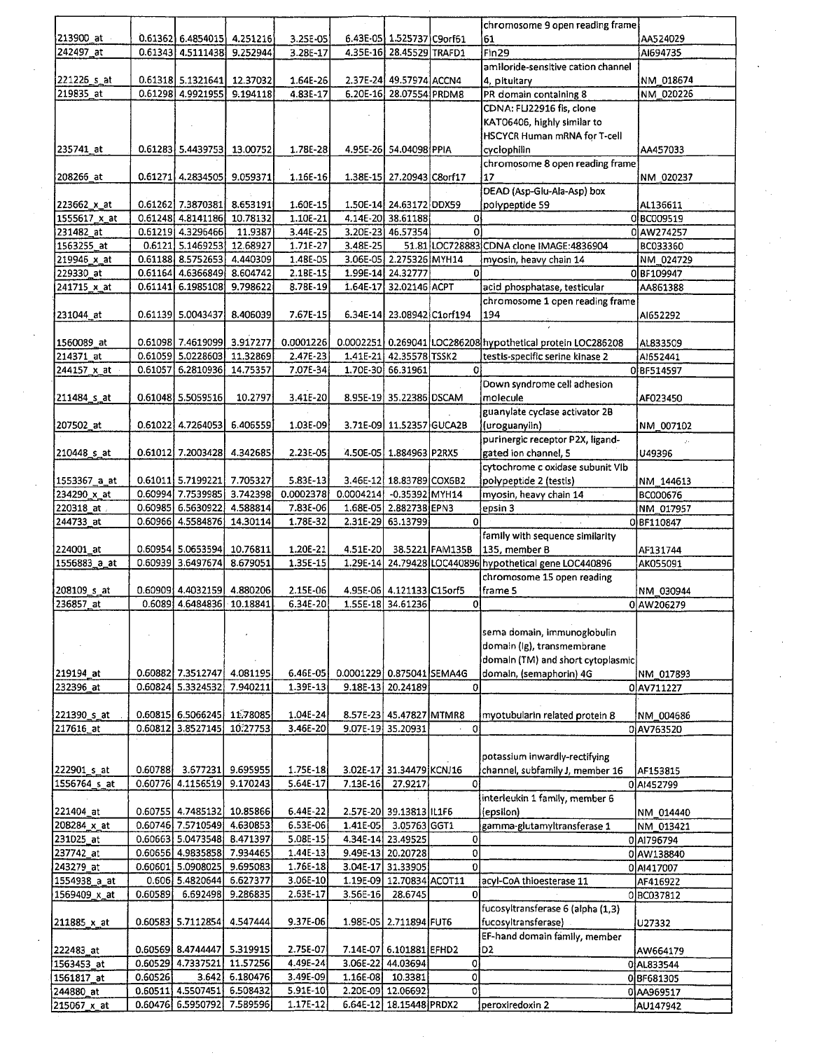

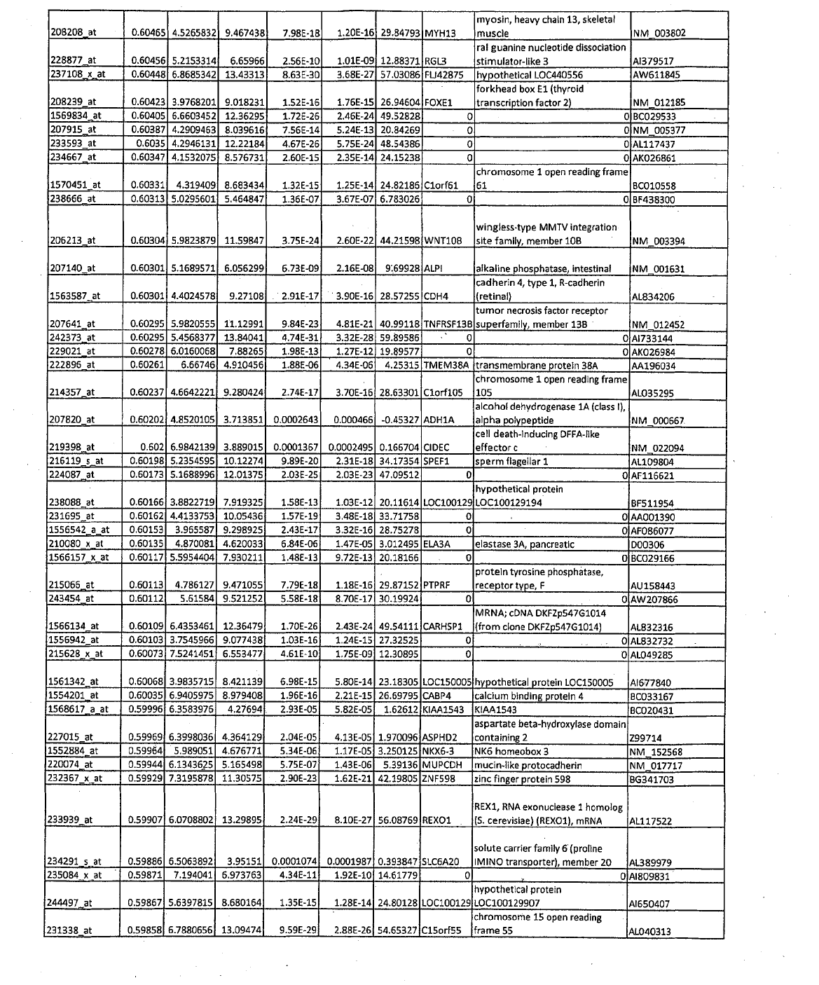

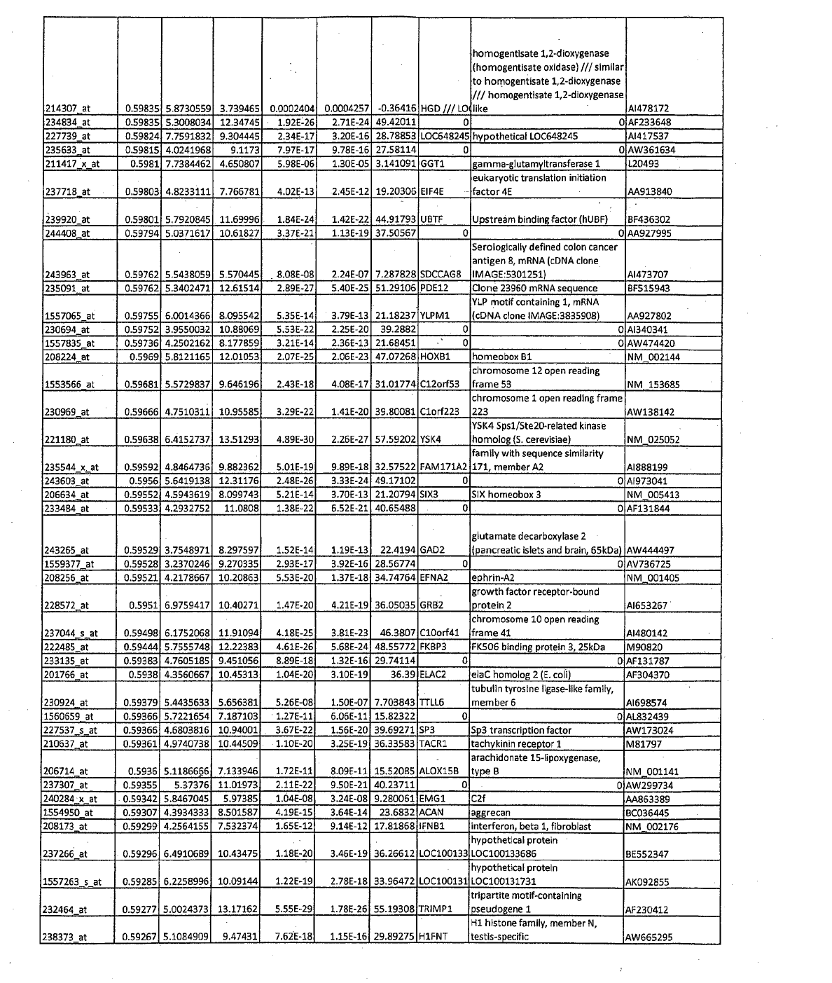

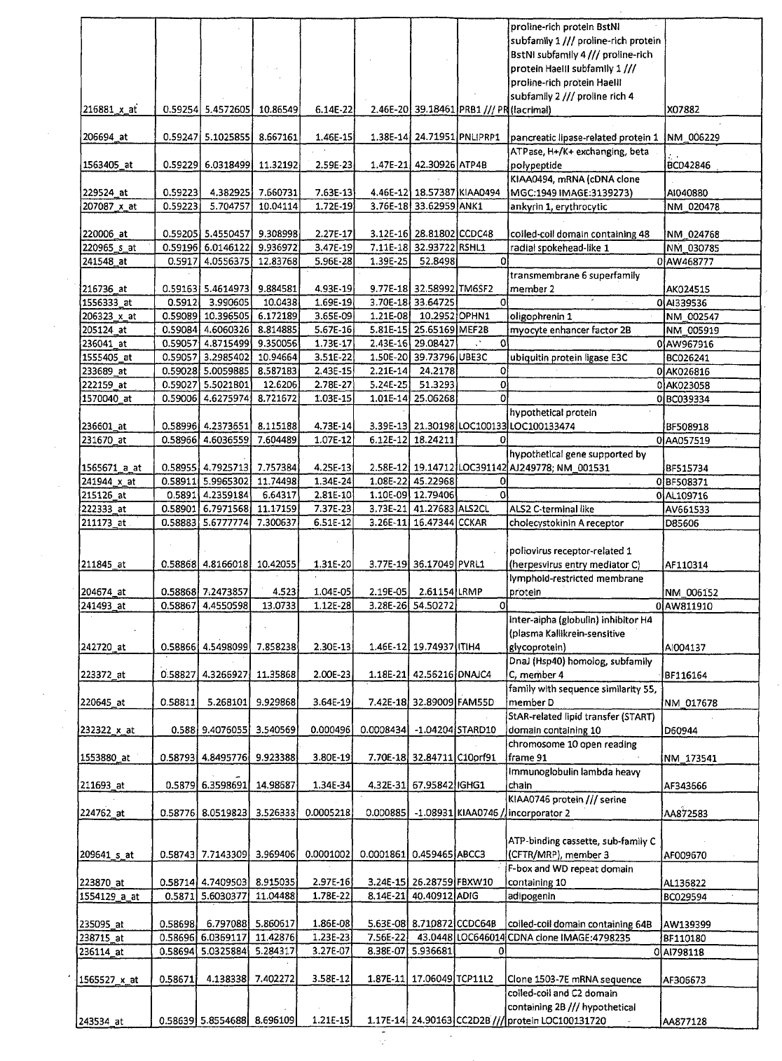

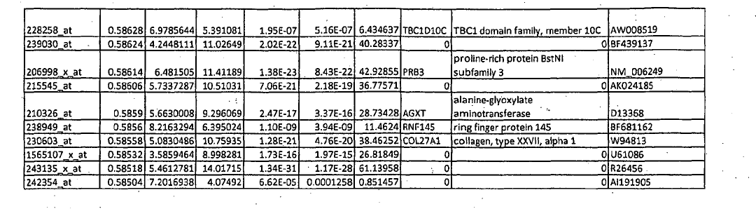

6. The grouping of claim 1, wherein the up-regulated genes referred to under a. of each subtype comprise the genes listed in FIG. 11 for the invasive subtype, in FIG. 12 for the proliferative subtype and in FIG. 13 for the metabolic subtype.

7. The grouping of claim 1, wherein
the cancer-stem-cell-like properties are characterized a) in that a pathway activity analysis shows that invasive subtype cancers are associated with activity of a cancer-stem-cell (CSC) gene set and with epithelial-mesenchymal transition conferring stem-cell-like properties; b) that CD44 expression is increased and CD24 expression is decreased compared to the proliferative subtype and the metabolic subtype; c) that it is associated with high-grade (that is, undifferentiated or poorly differentiated) gastric cancers; and d) that it is sensitive to compounds inhibiting the PI3K/AKT/mTOR pathway.

8. A predictor for classifying a patient based on the gene expression profile to one of the gastric cancer subtypes referred to in claim 1,
wherein the predictor comprises an ensemble of three predictors, wherein each of the three predictors comprises genes that are differentially expressed between one pair of the subtypes referred to in claim 1.

9. The predictor of claim 8, wherein genes were considered differentially expressed between two subtypes if the false discovery rate (FDR) is <0.001 and the absolute fold change is >1.5.

10. The predictor of claim 8, wherein the predictor is based on the nearest template prediction.

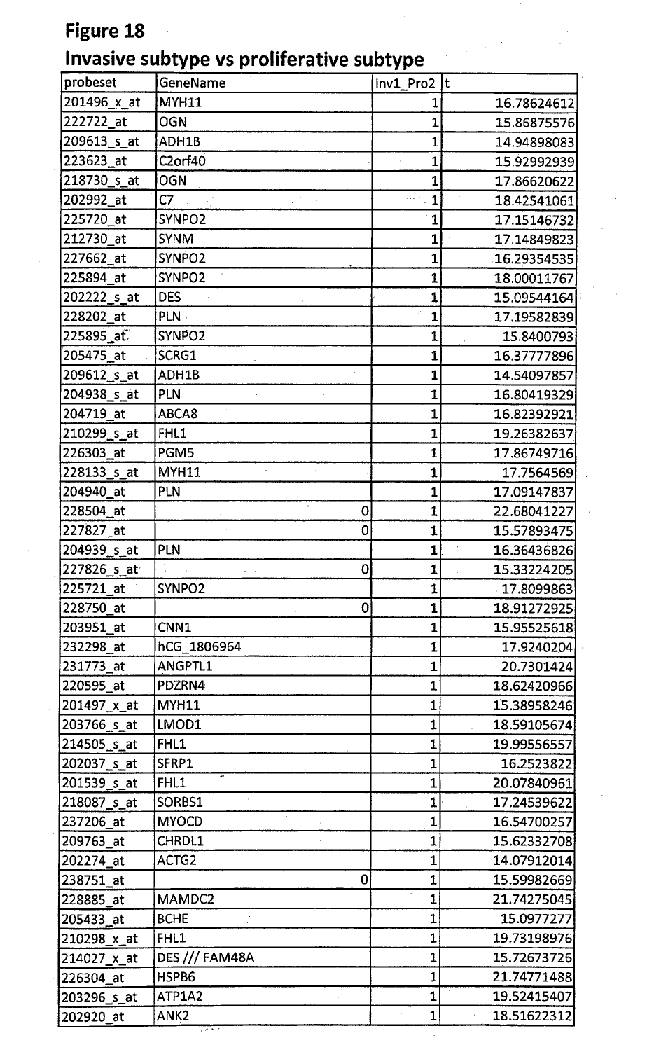

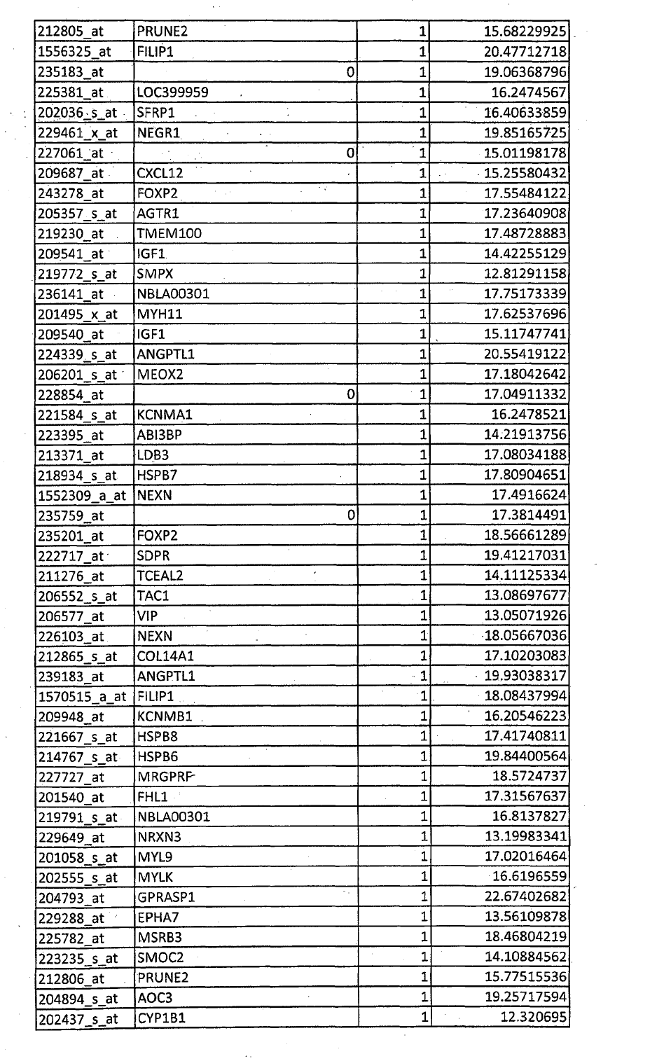

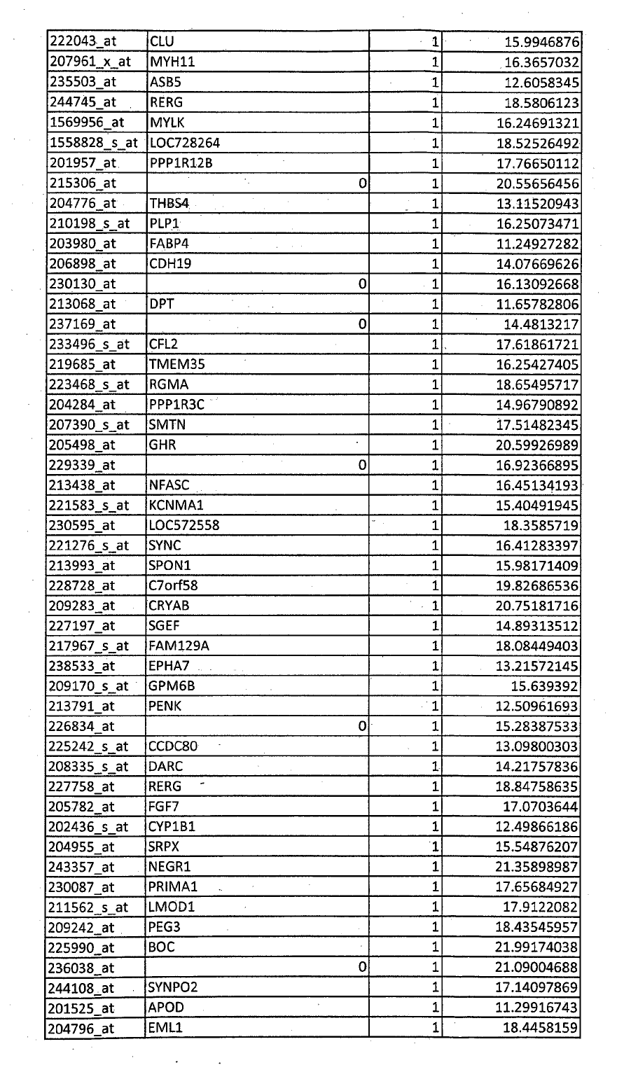

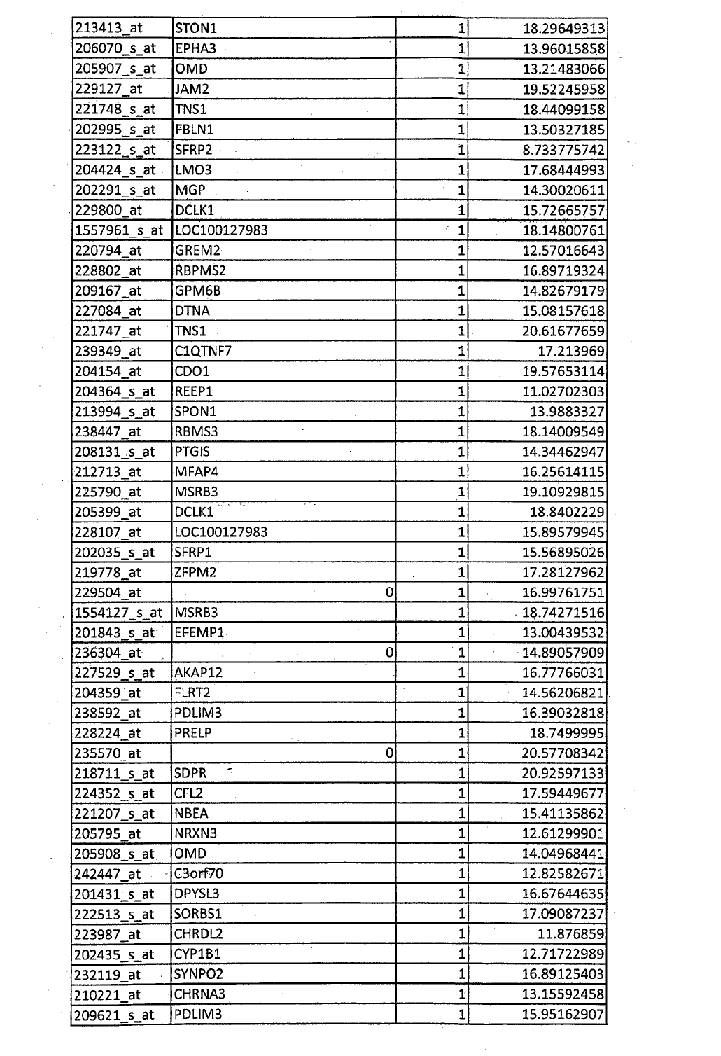

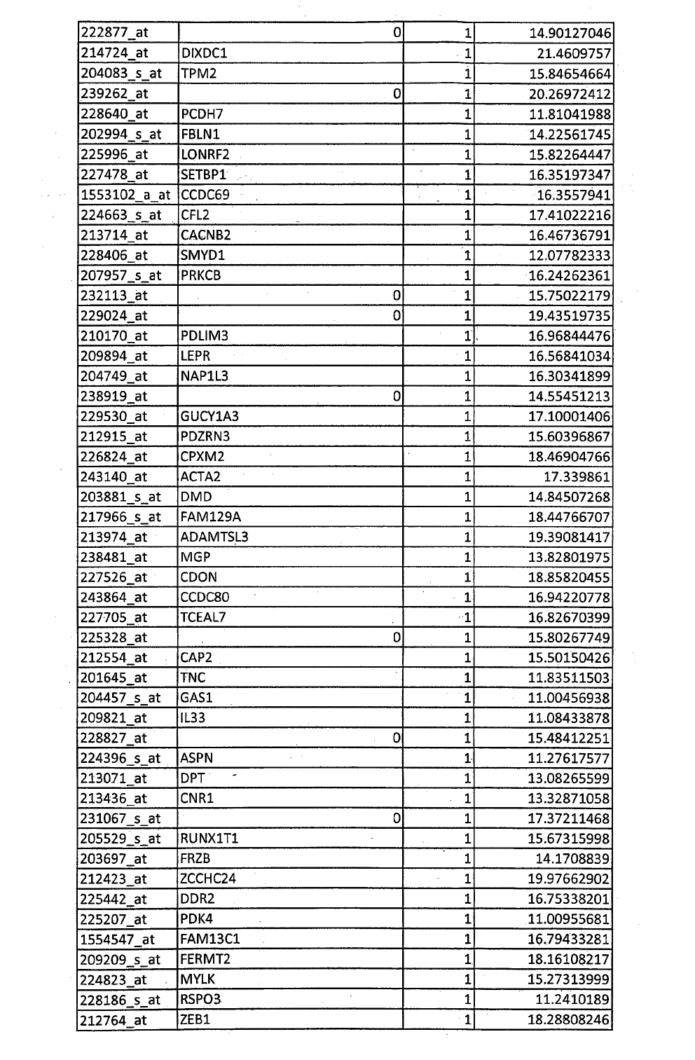

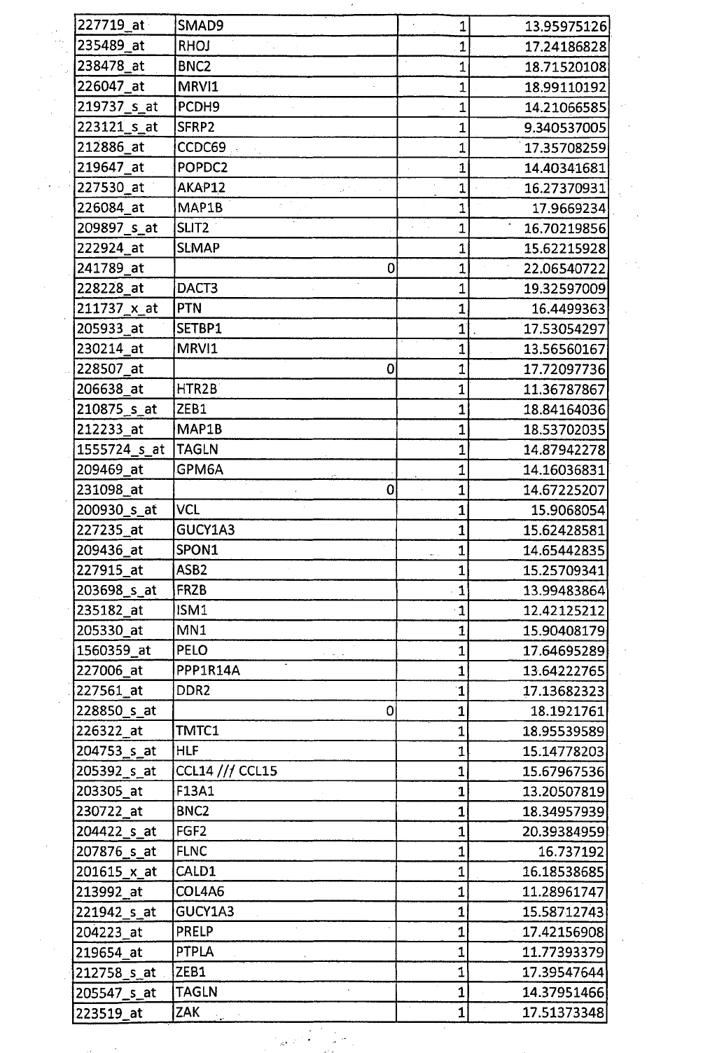

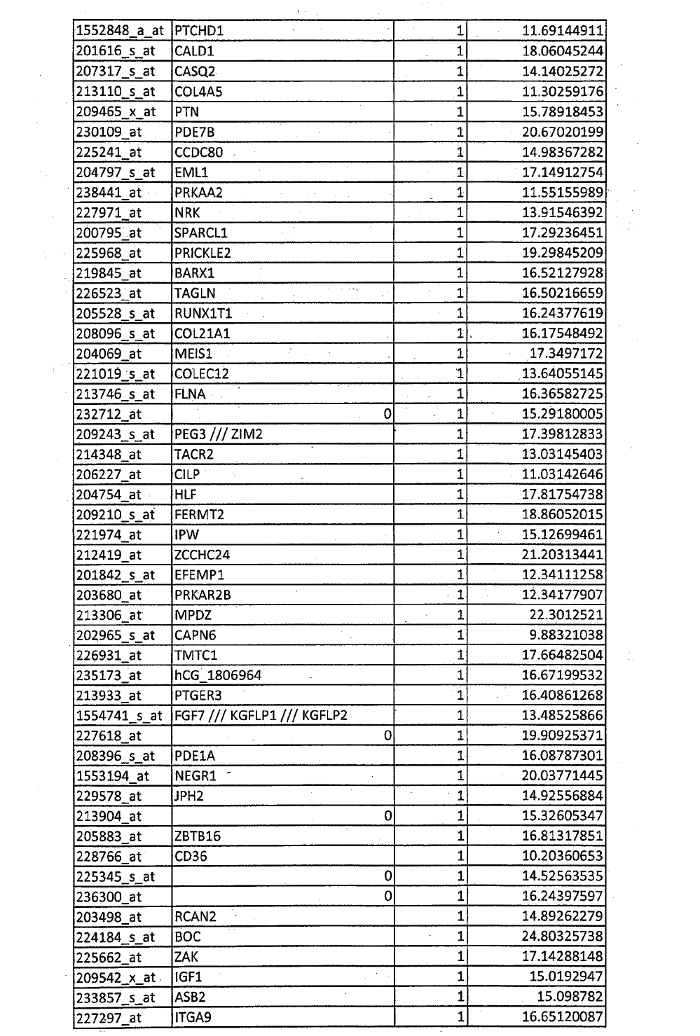

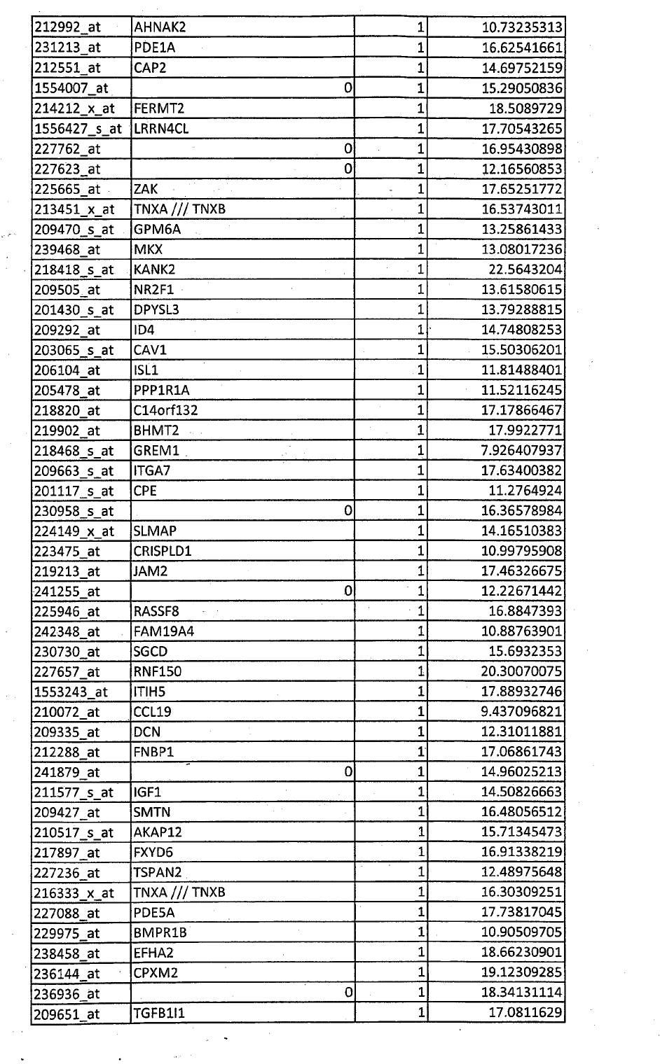

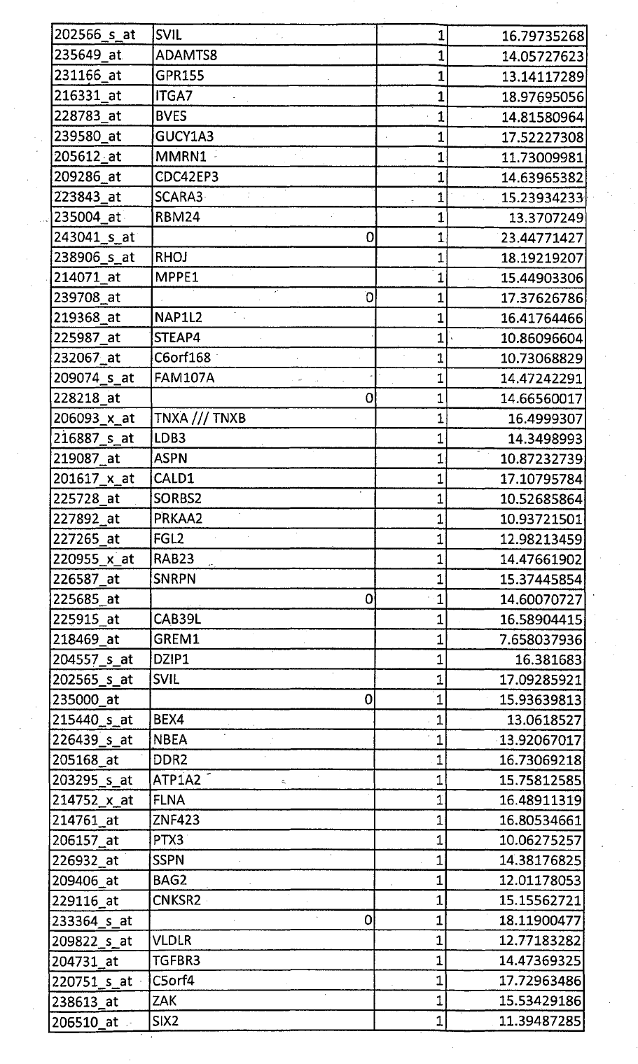

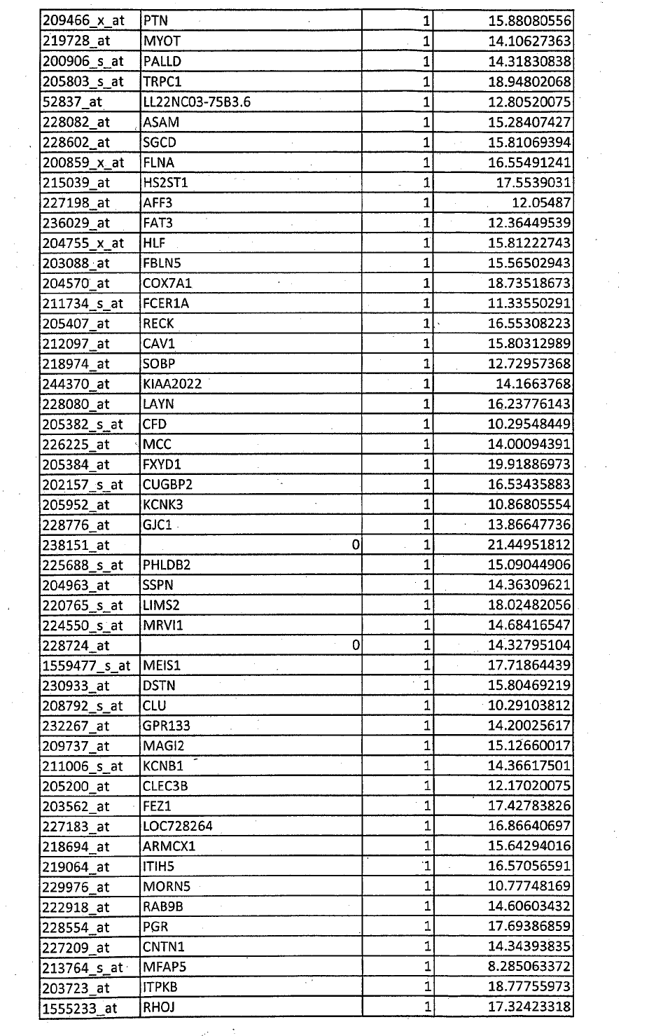

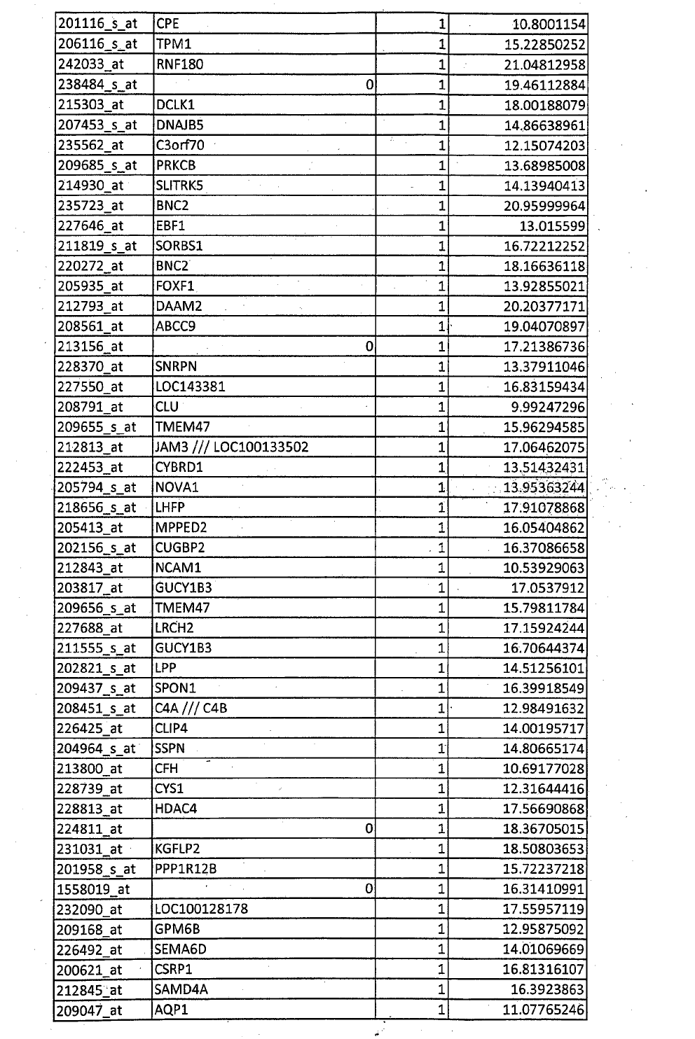

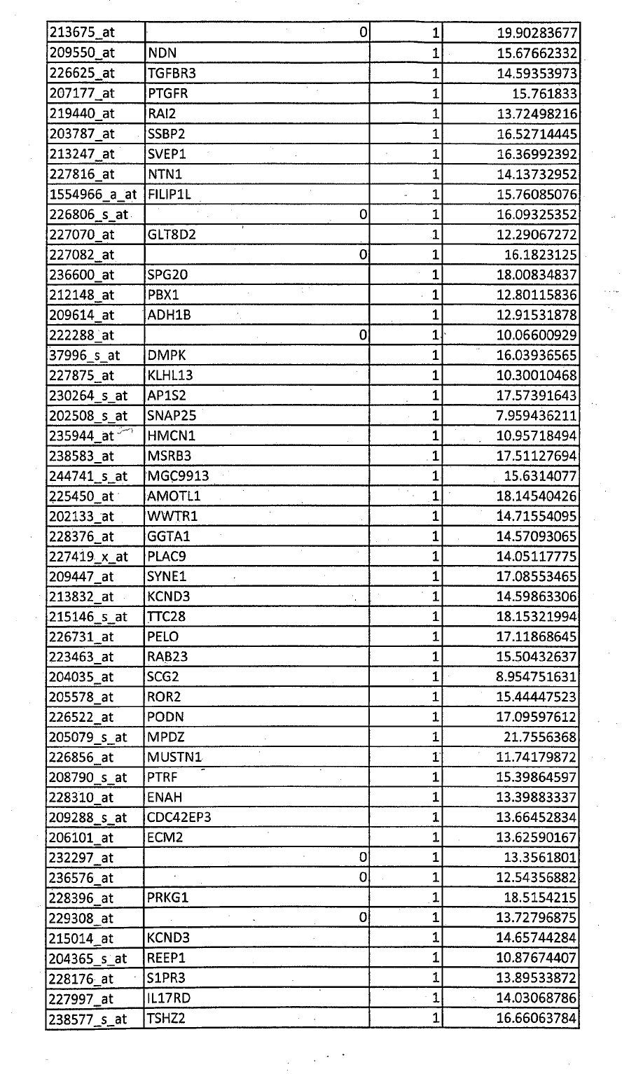

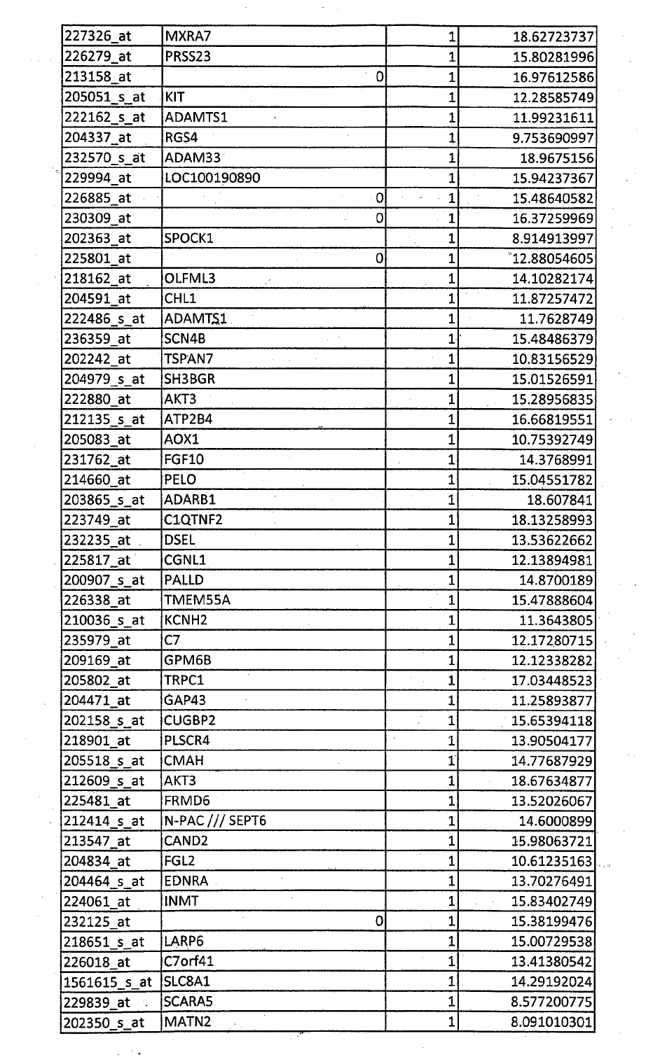

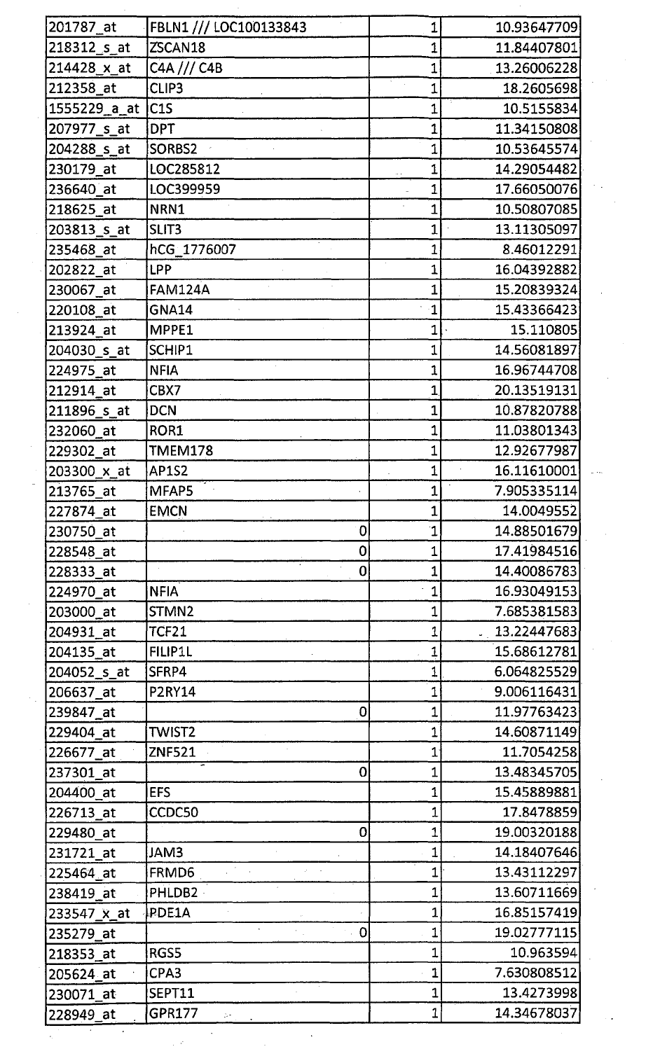

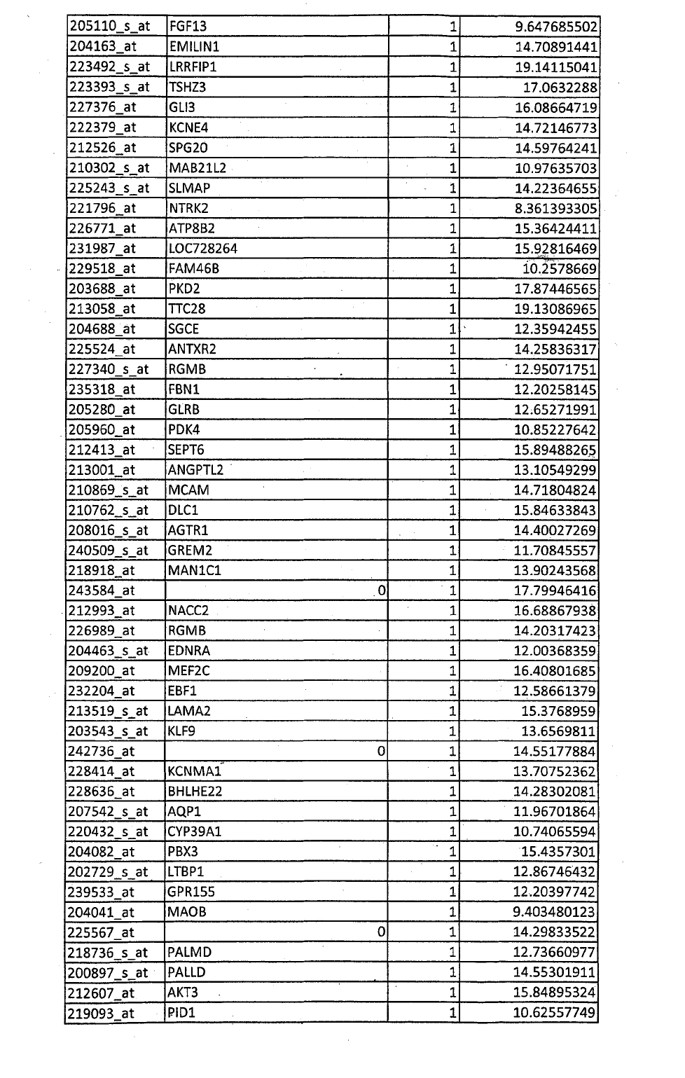

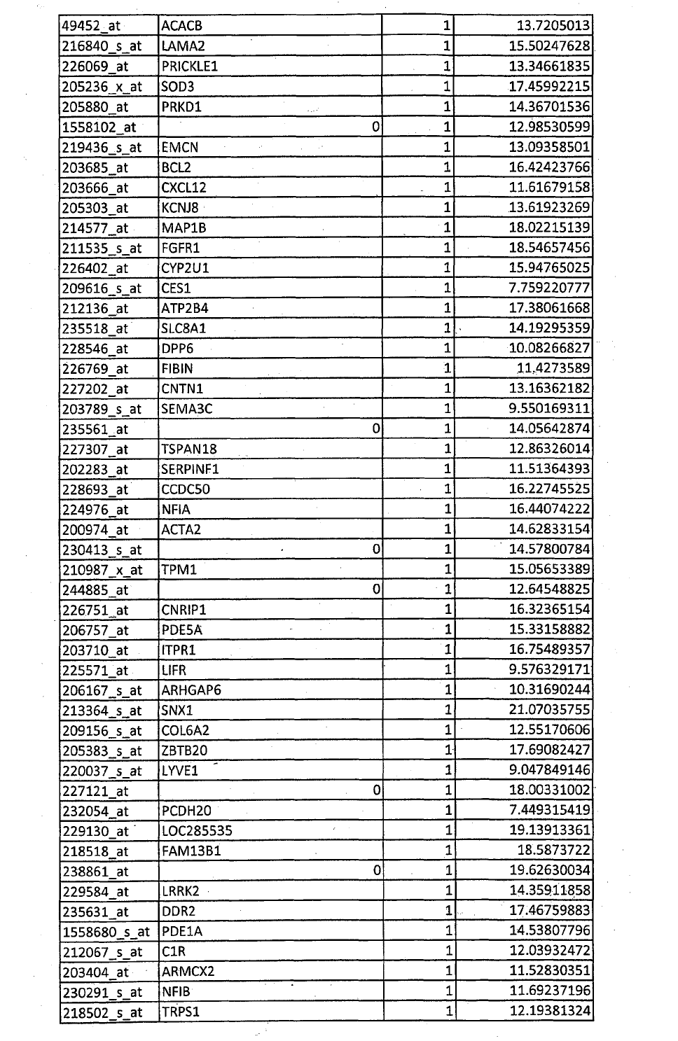

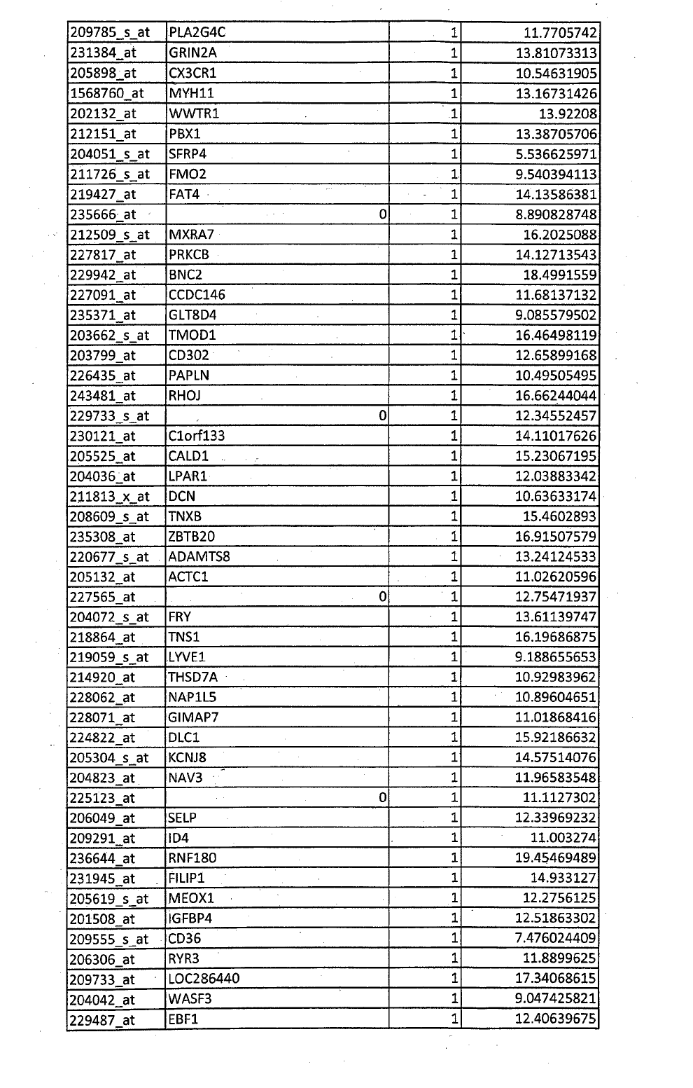

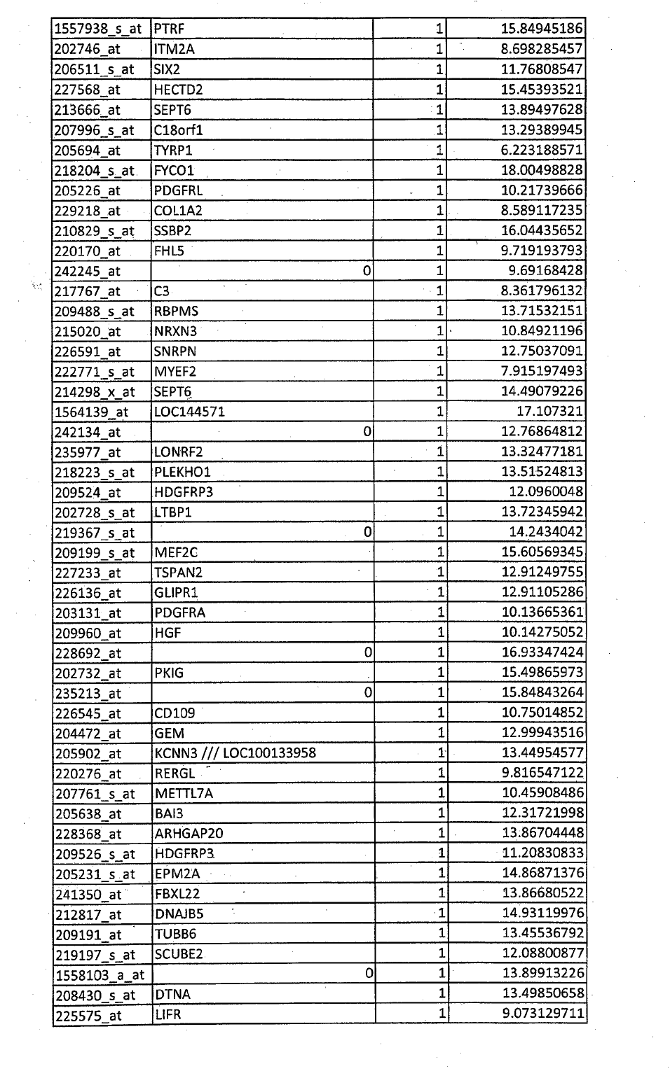

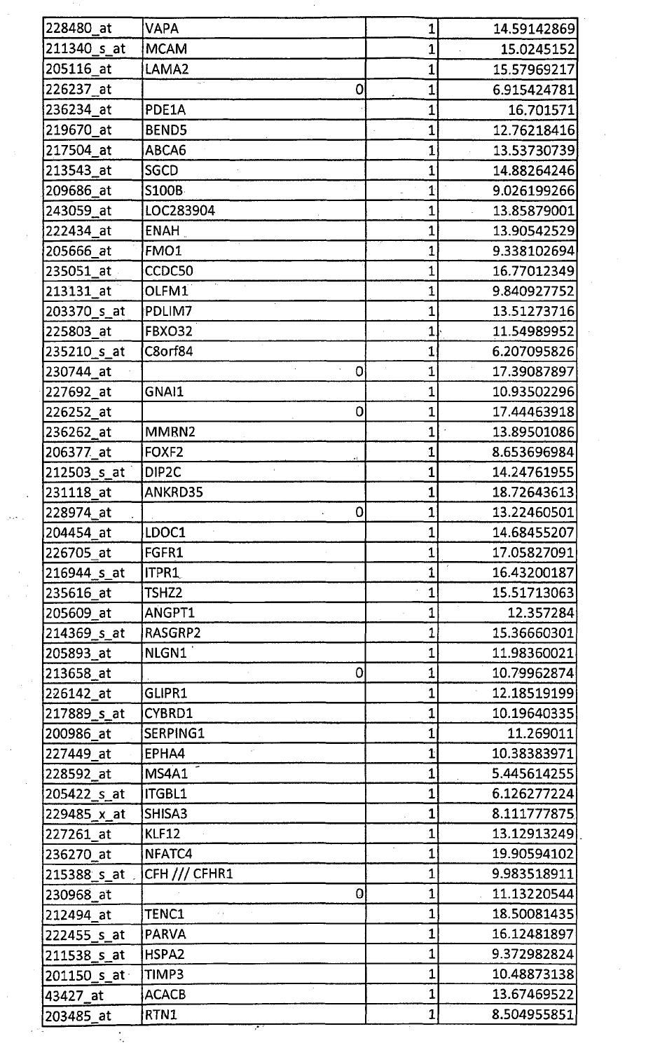

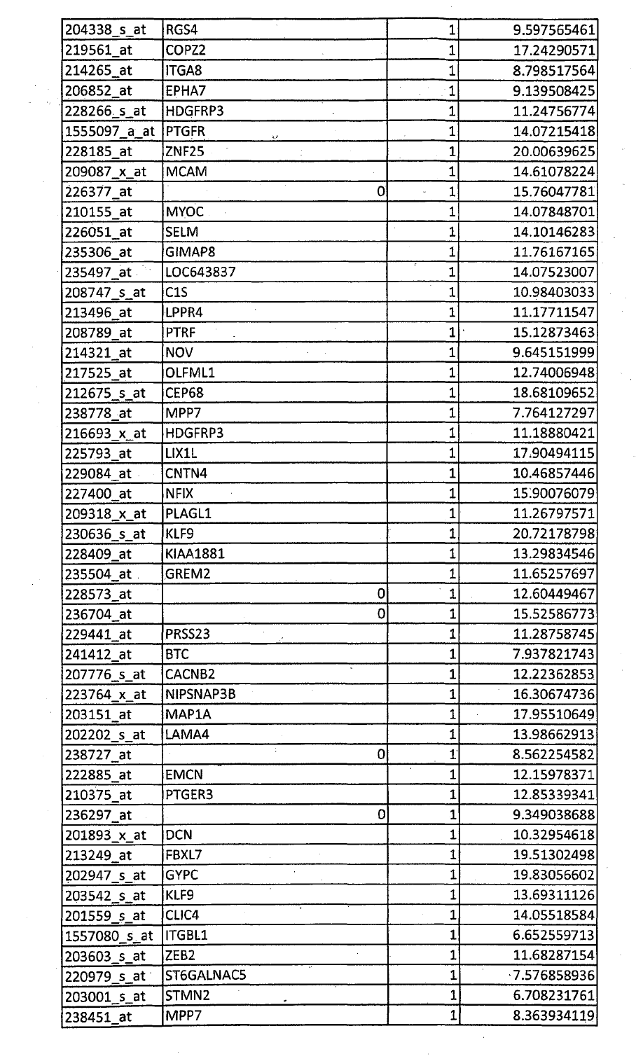

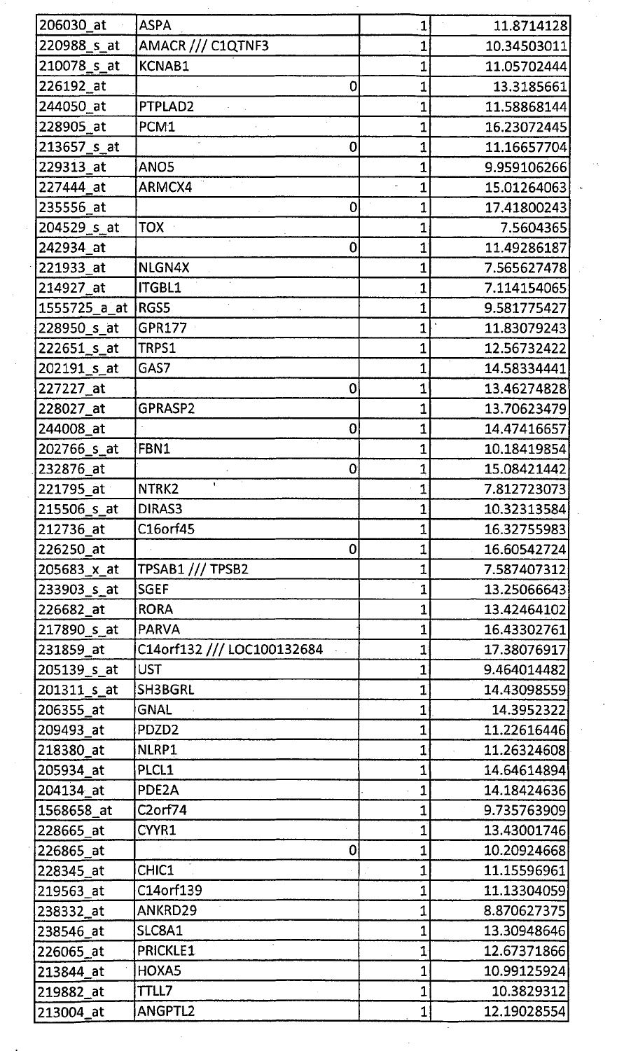

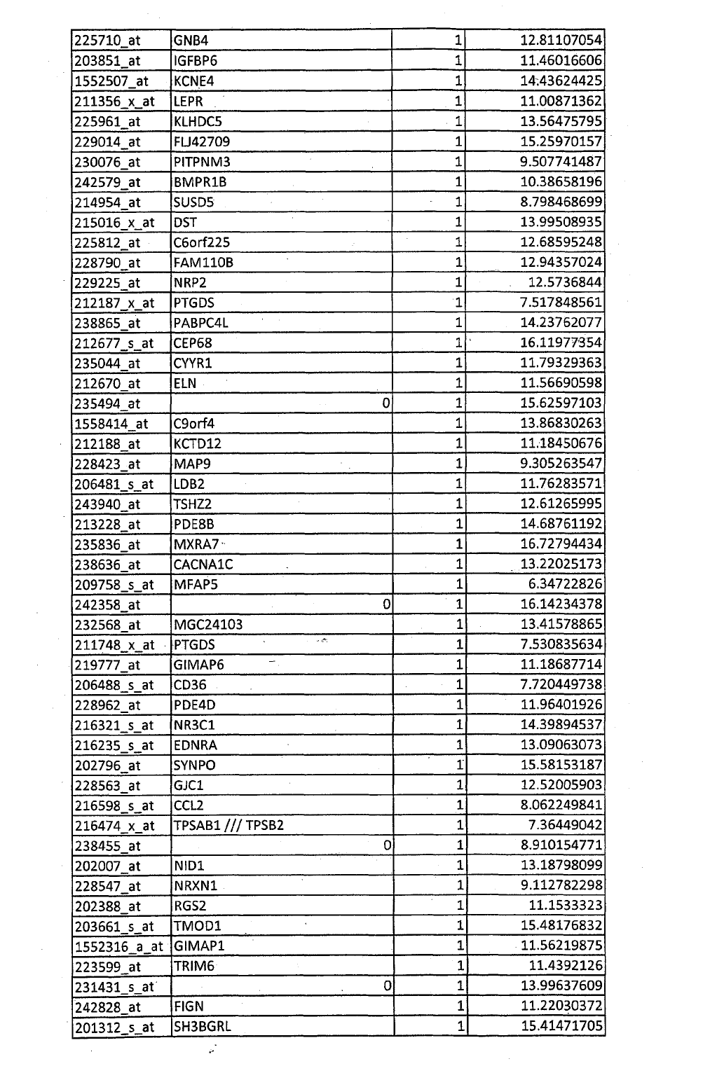

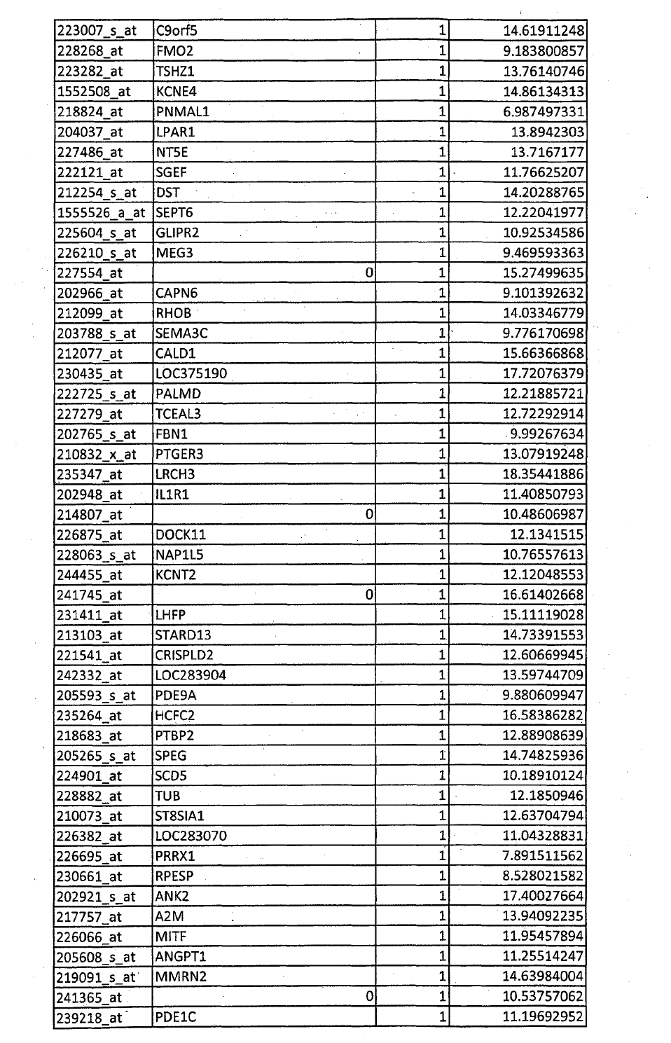

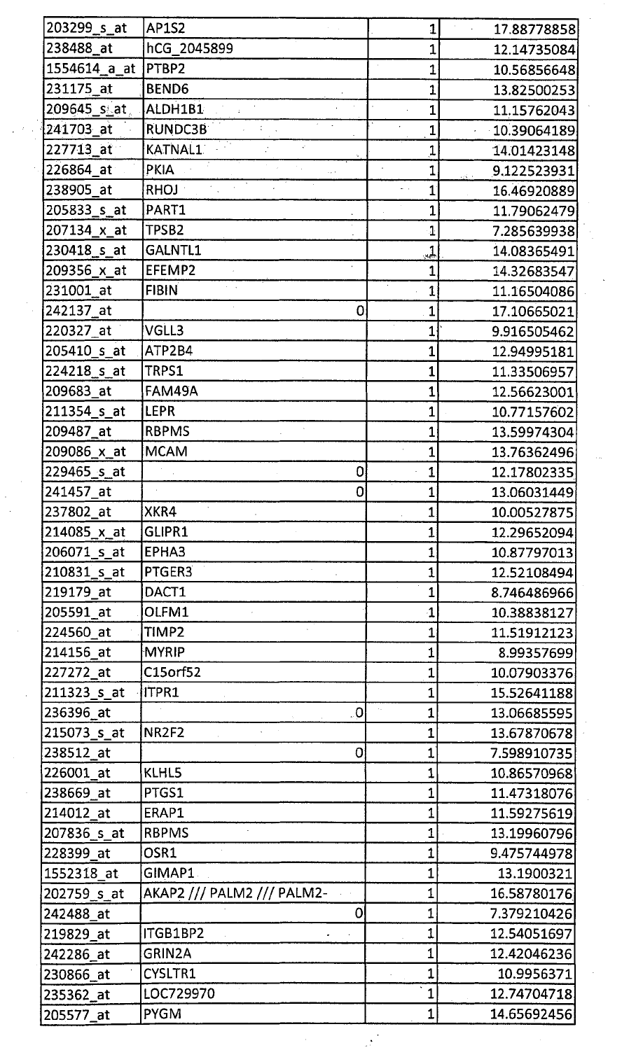

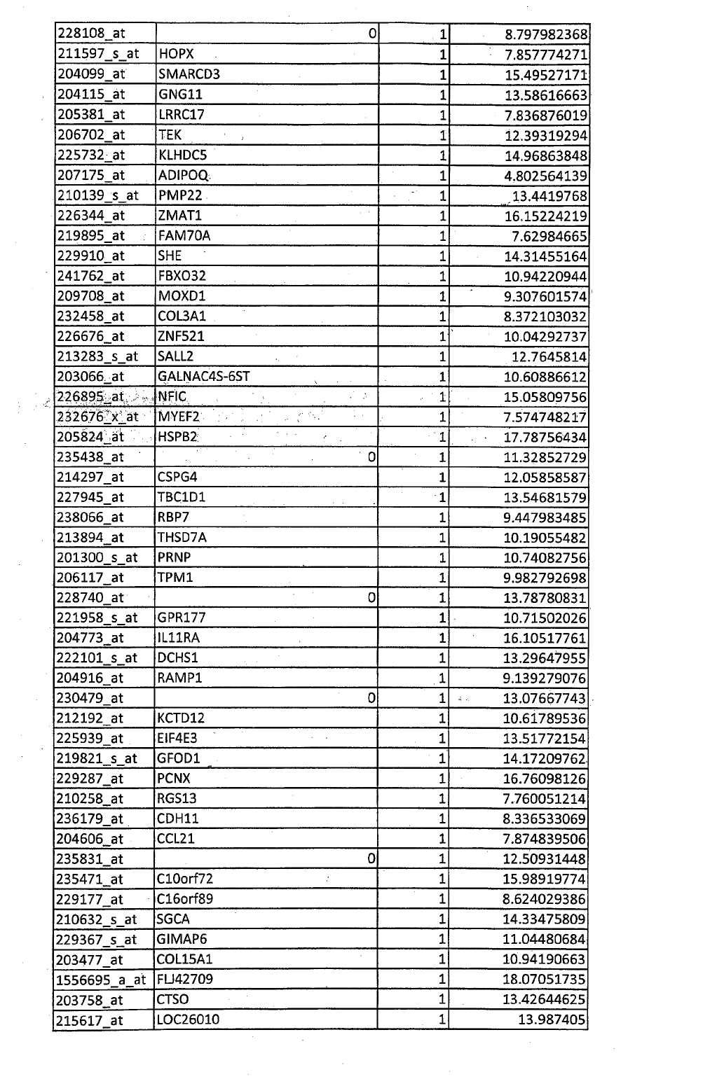

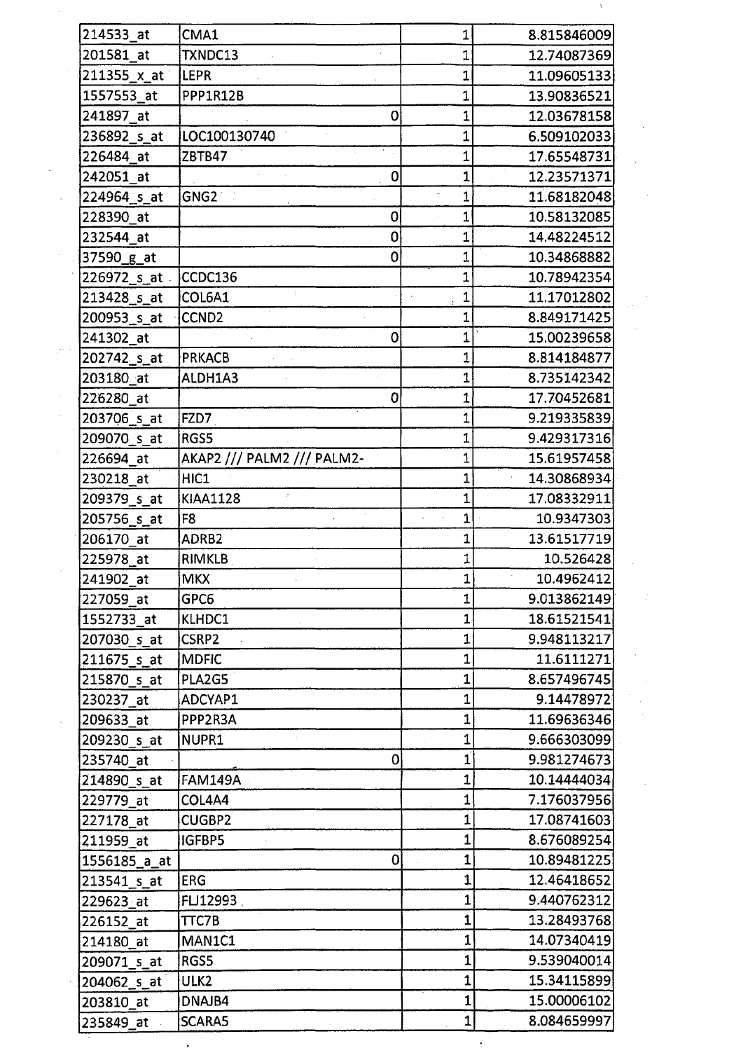

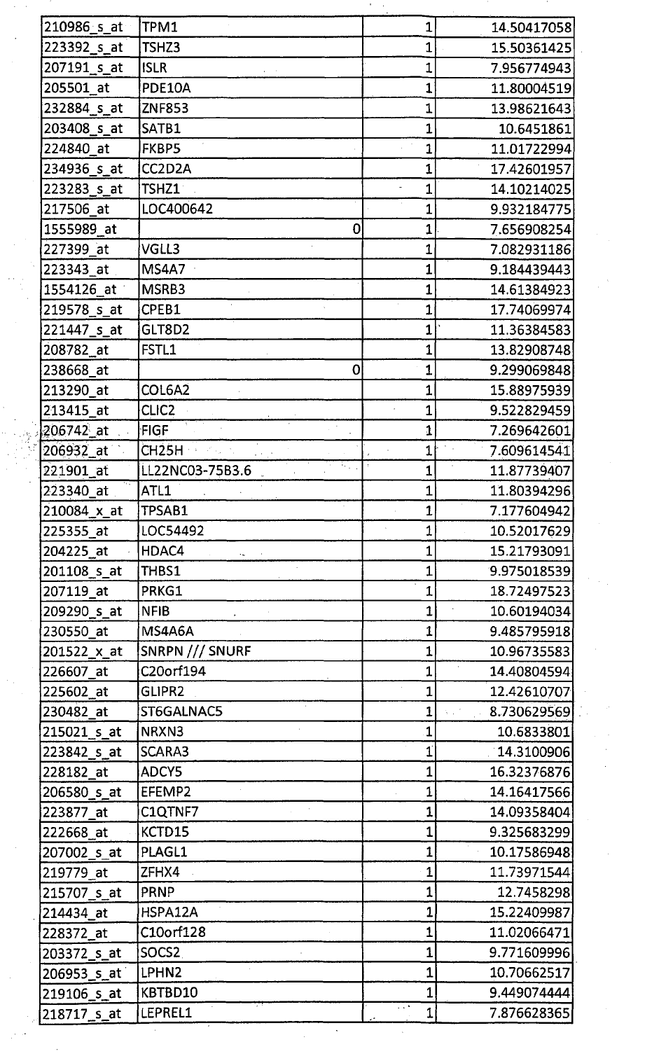

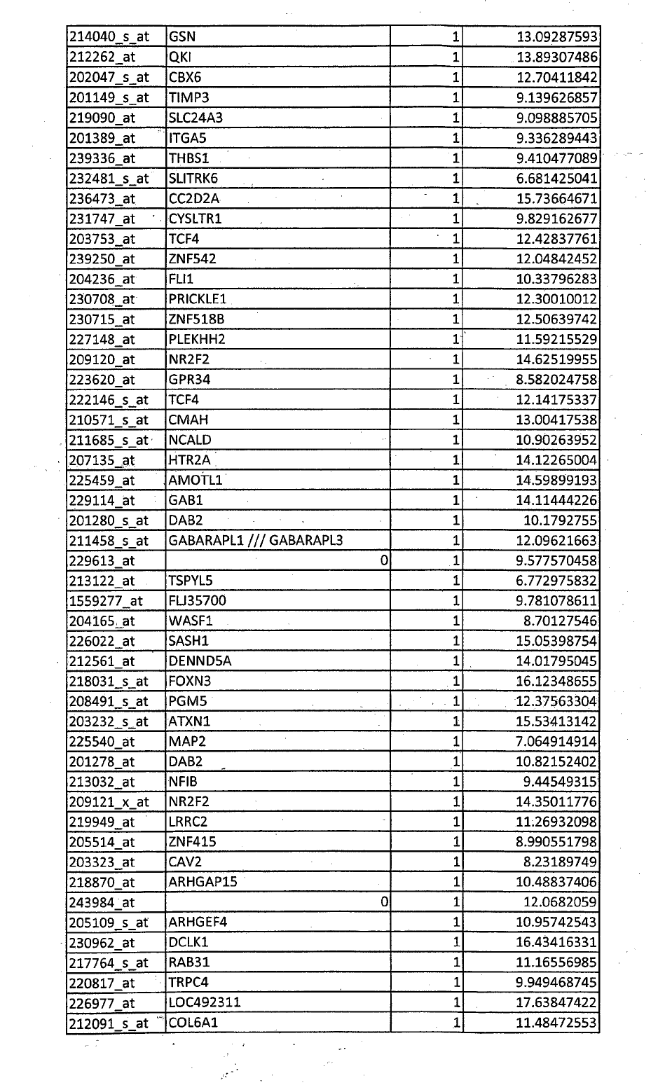

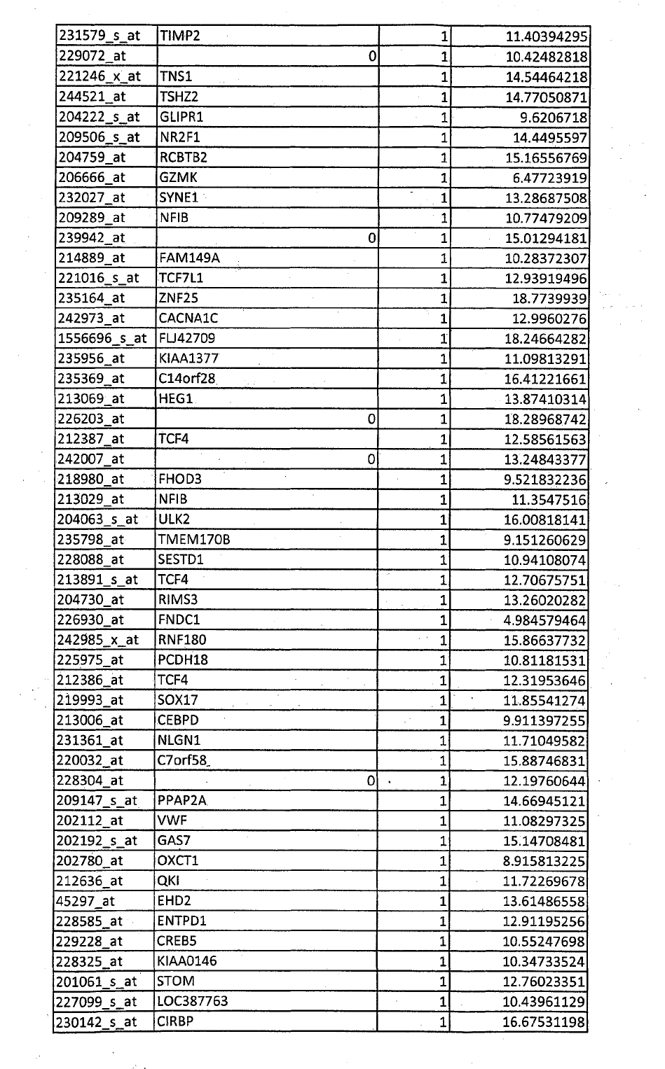

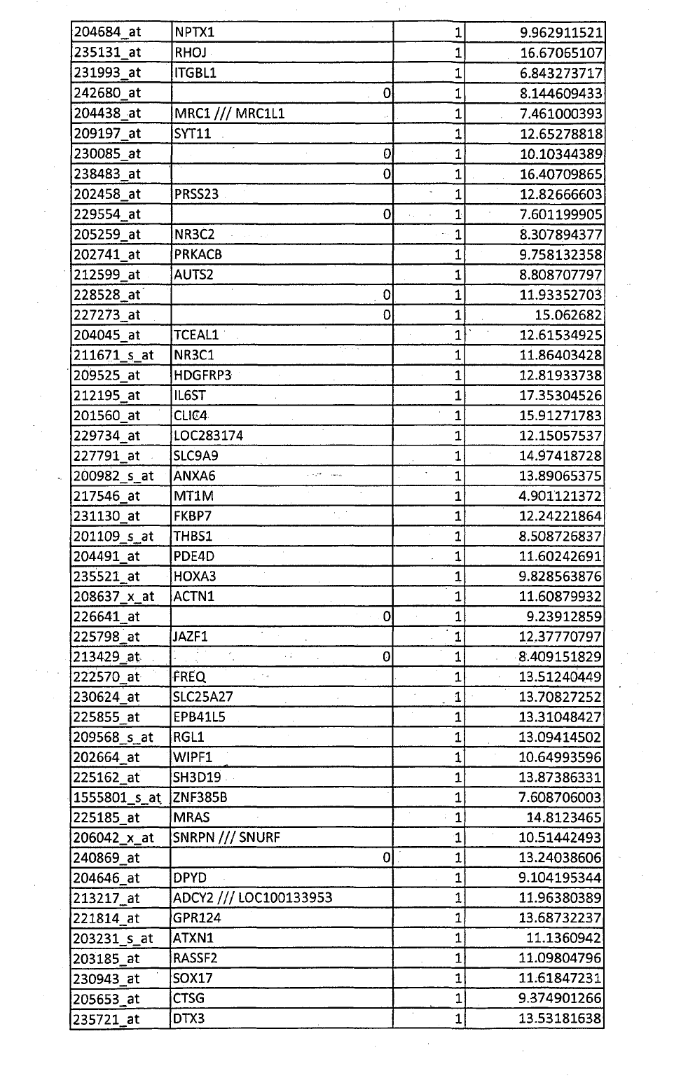

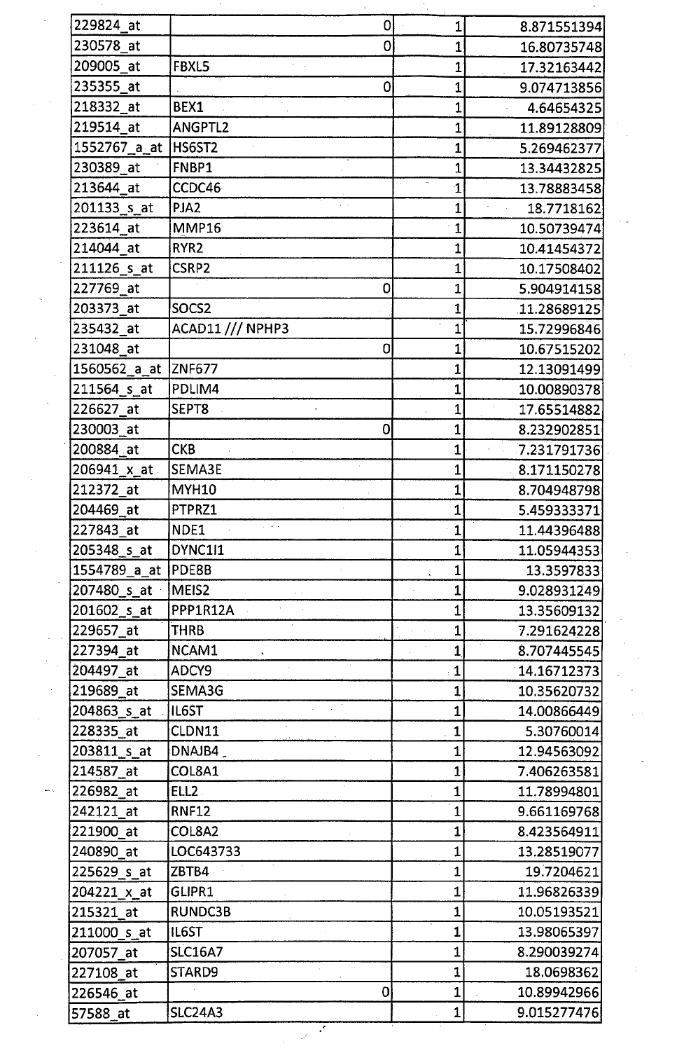

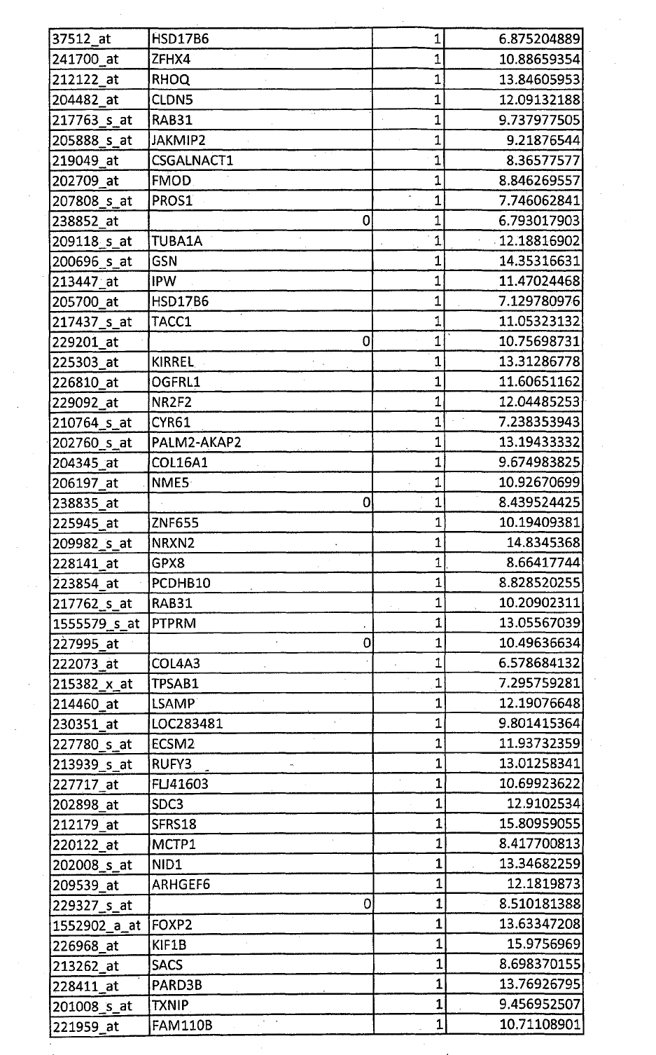

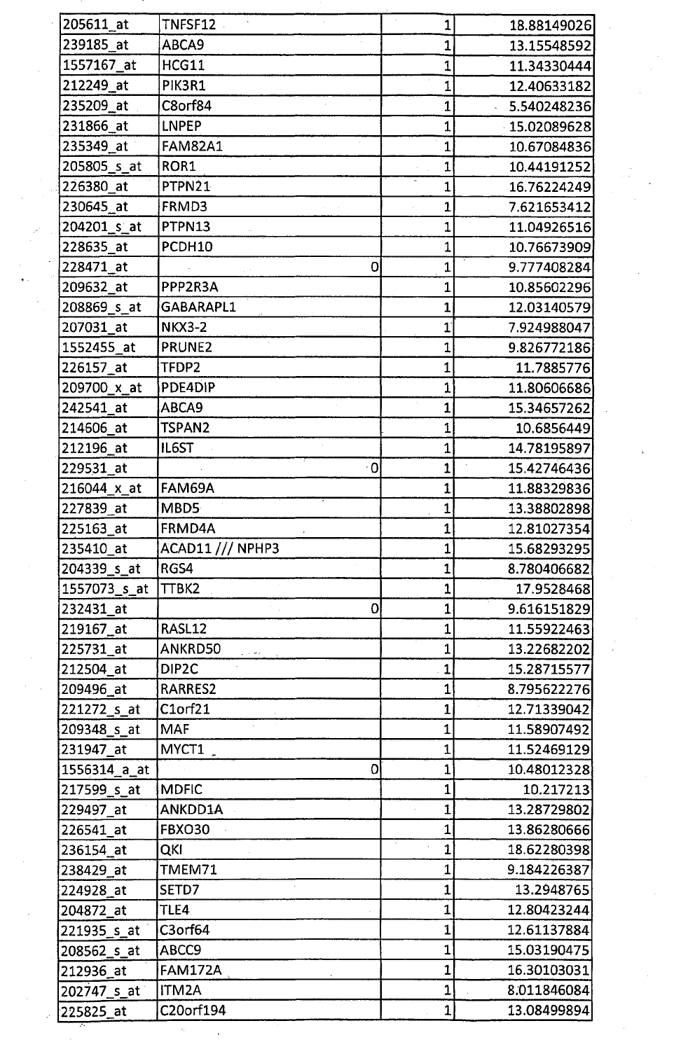

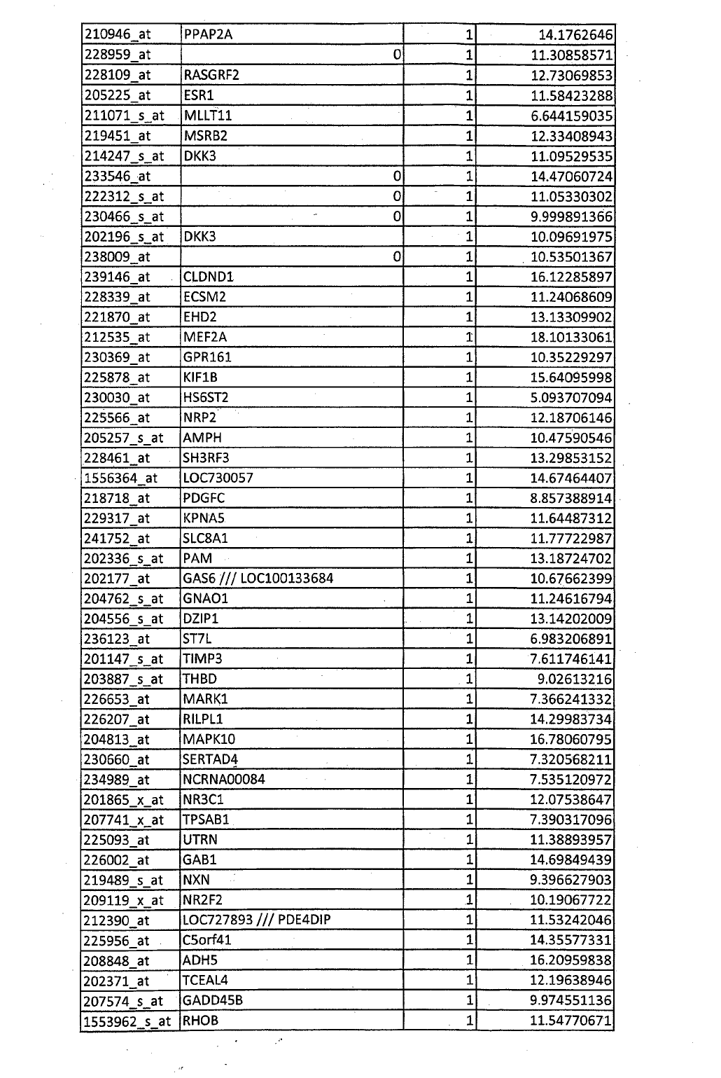

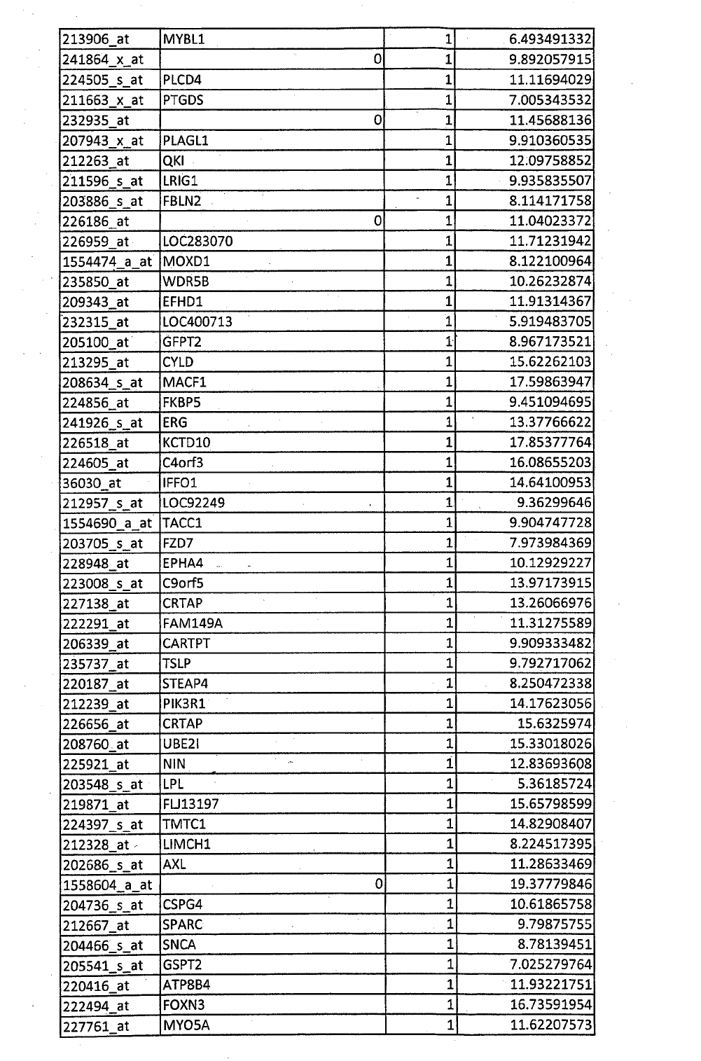

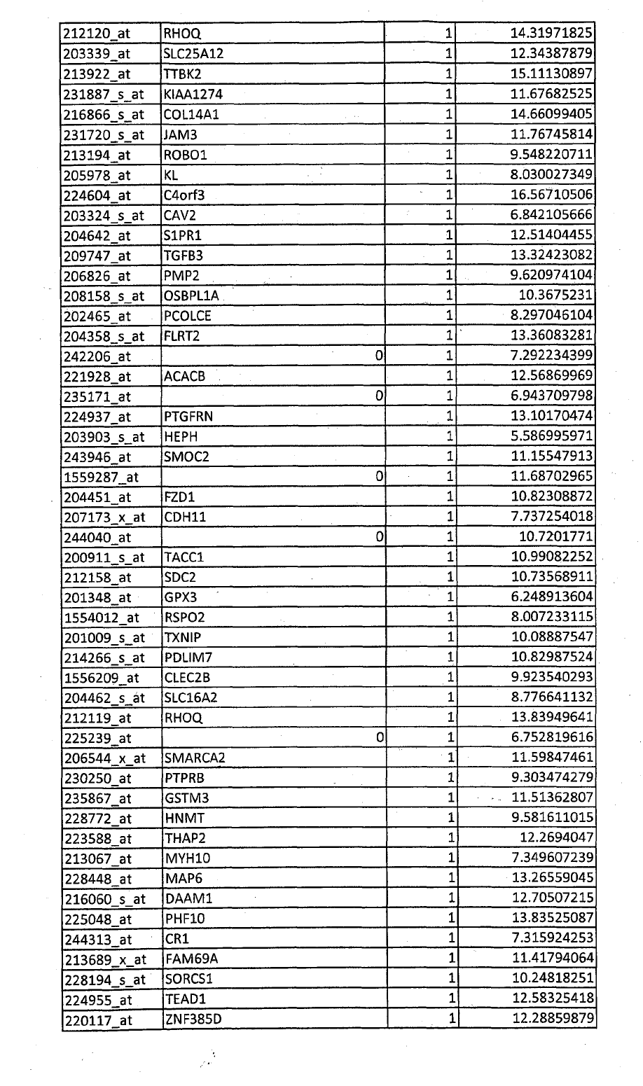

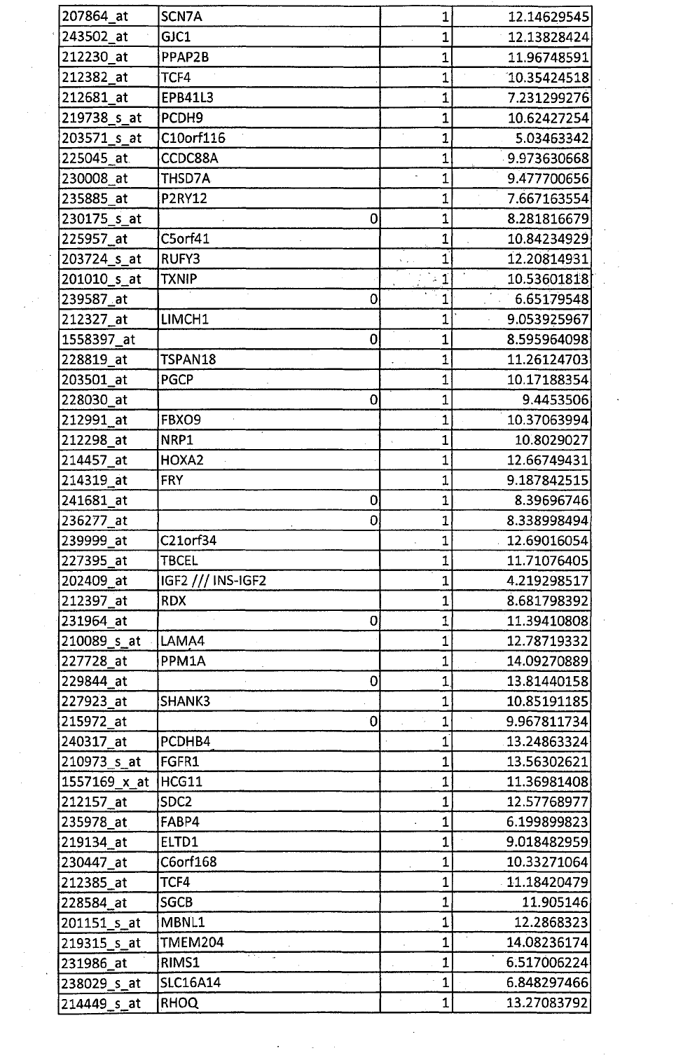

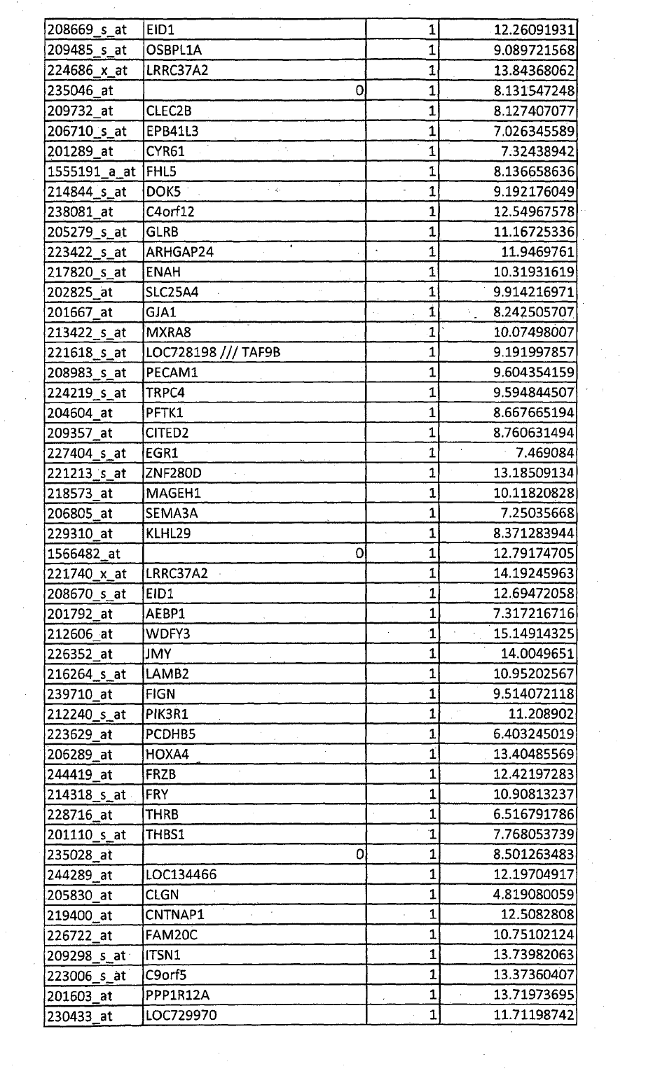

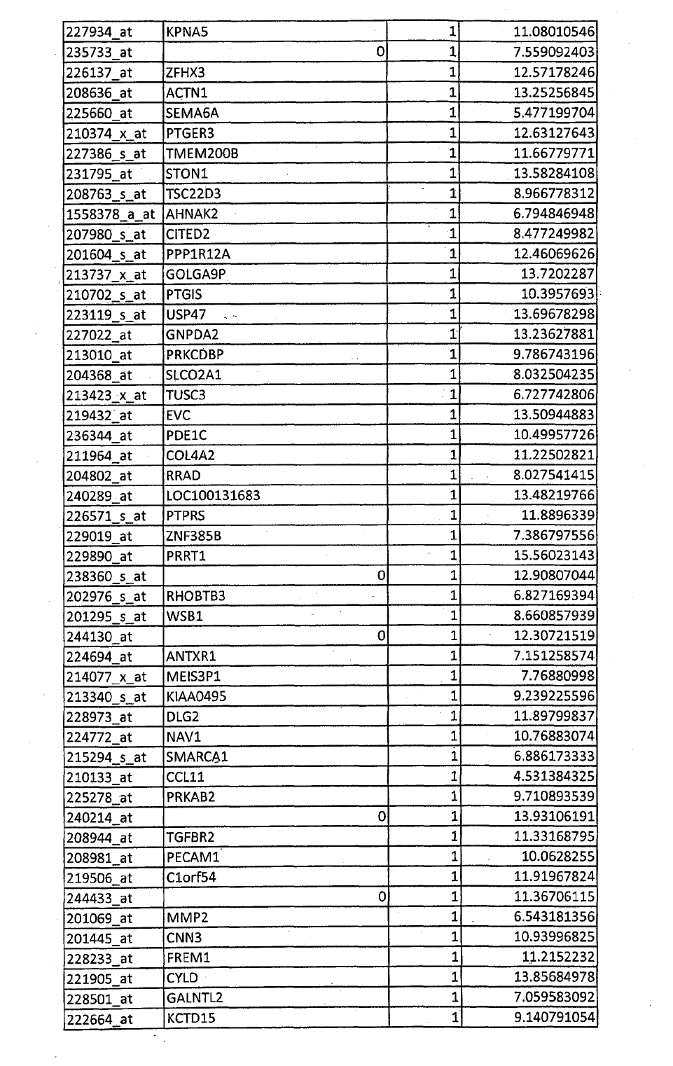

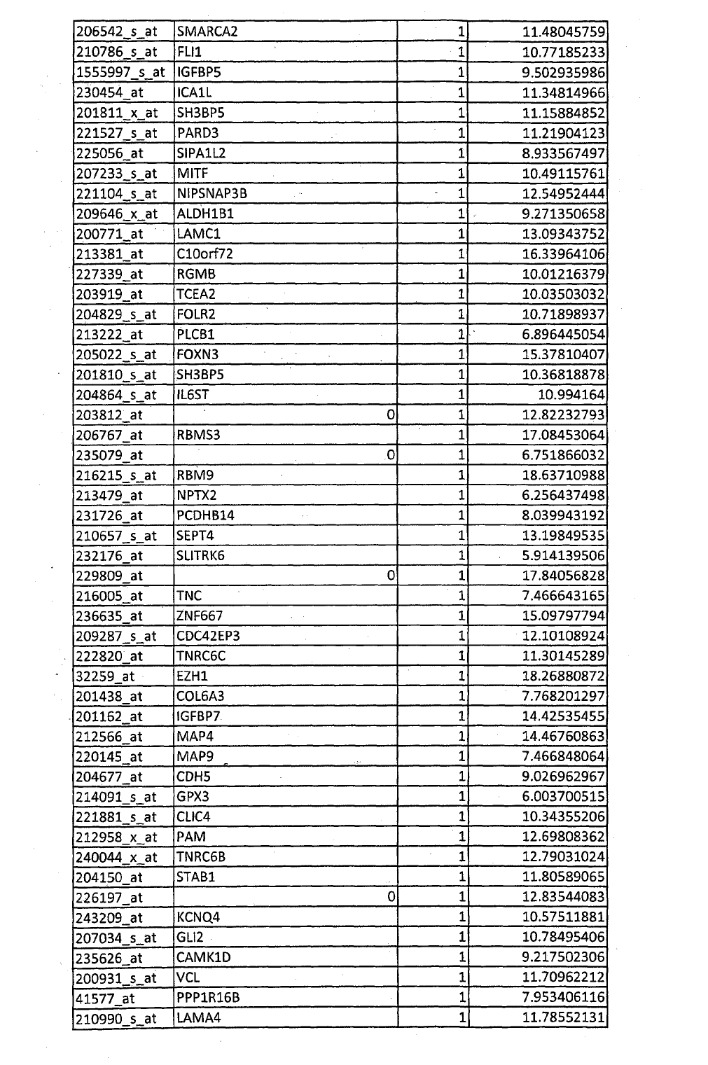

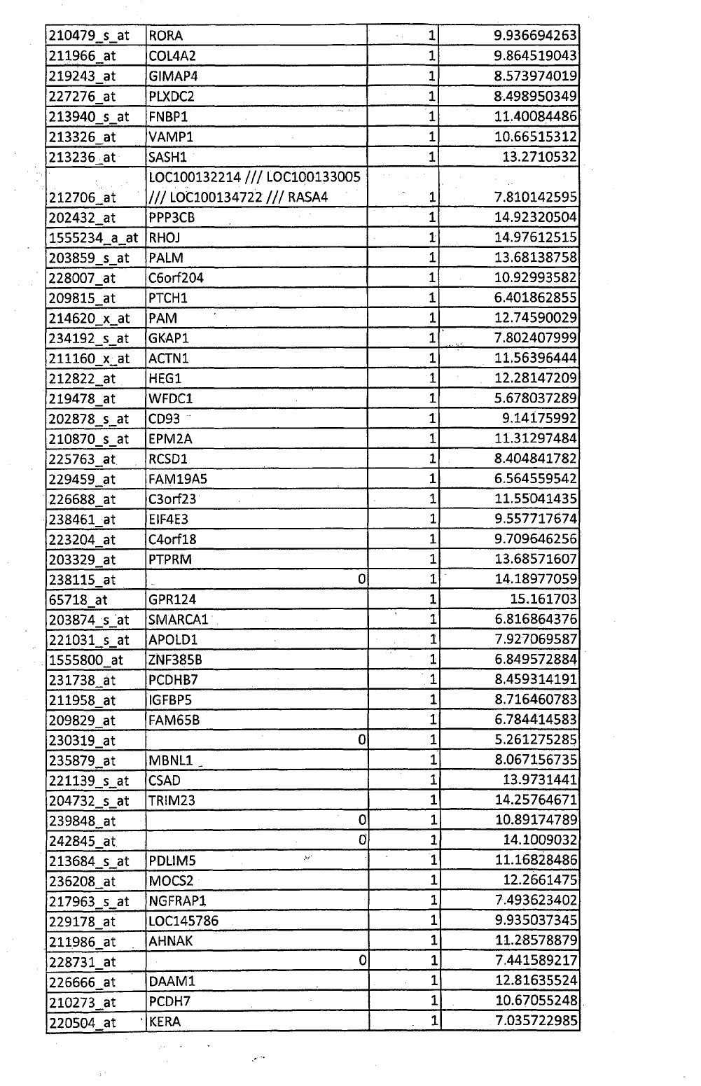

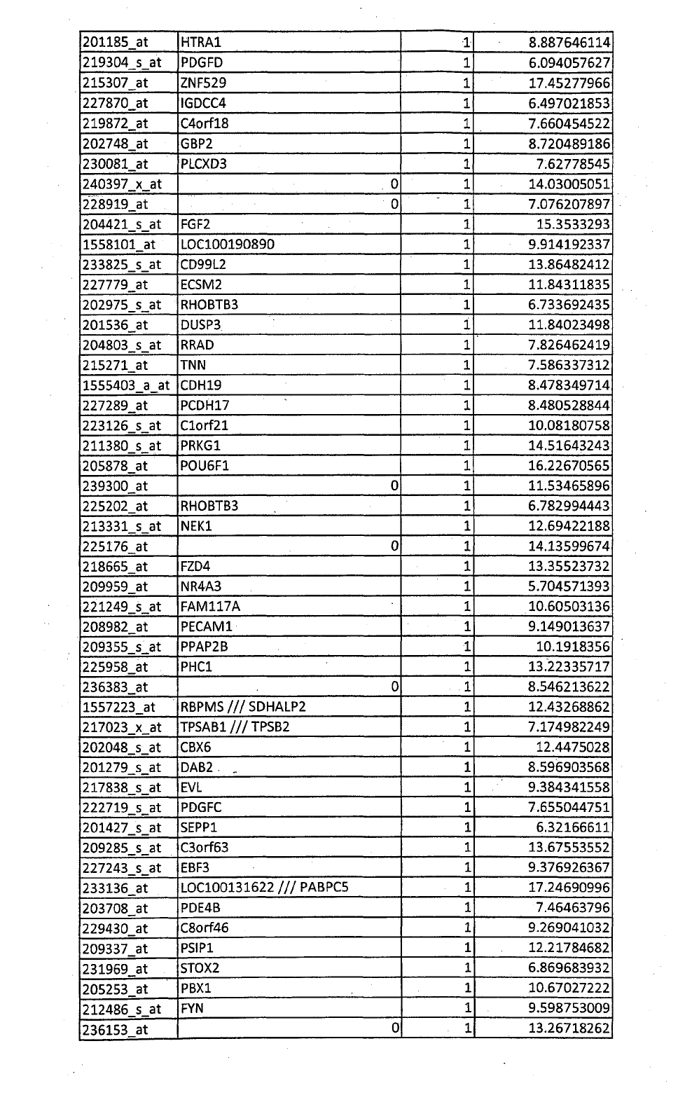

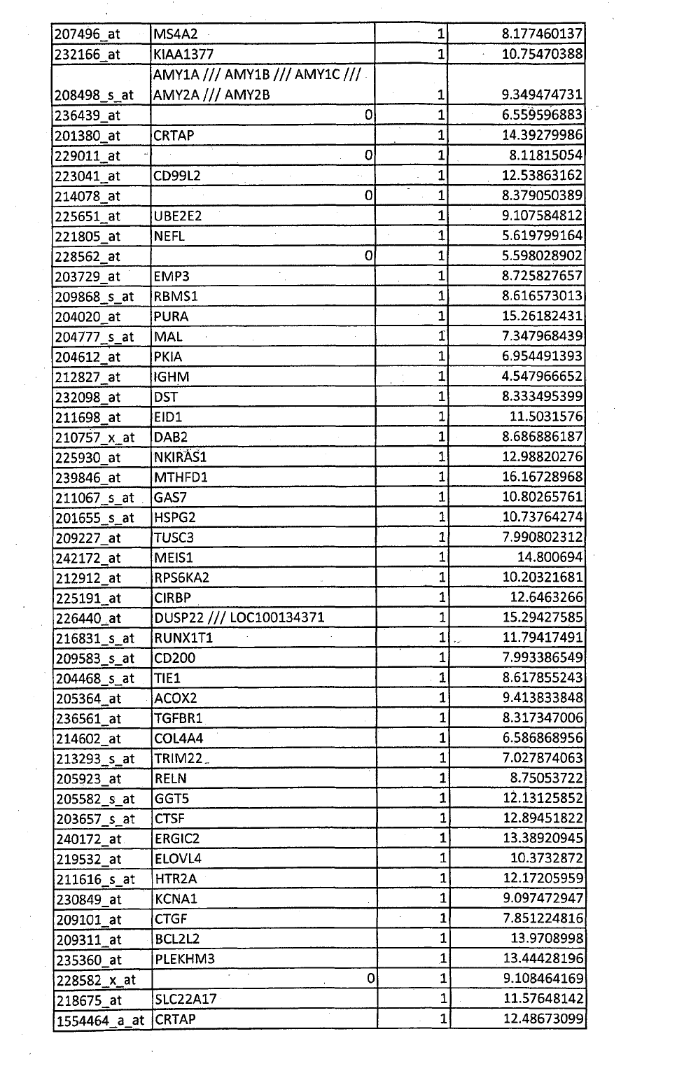

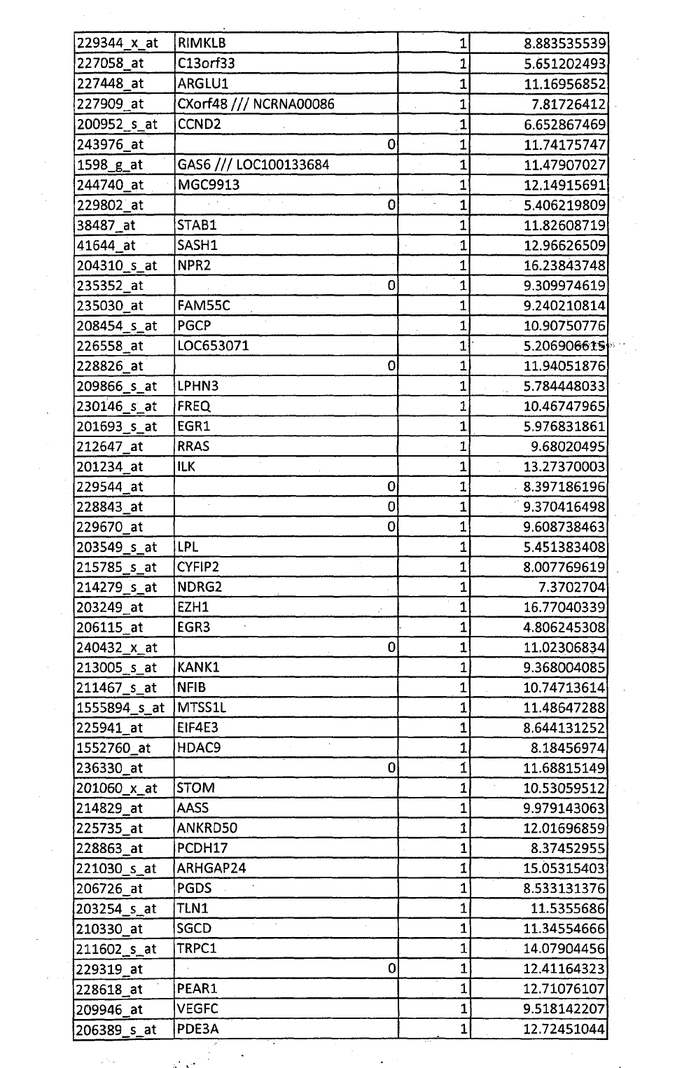

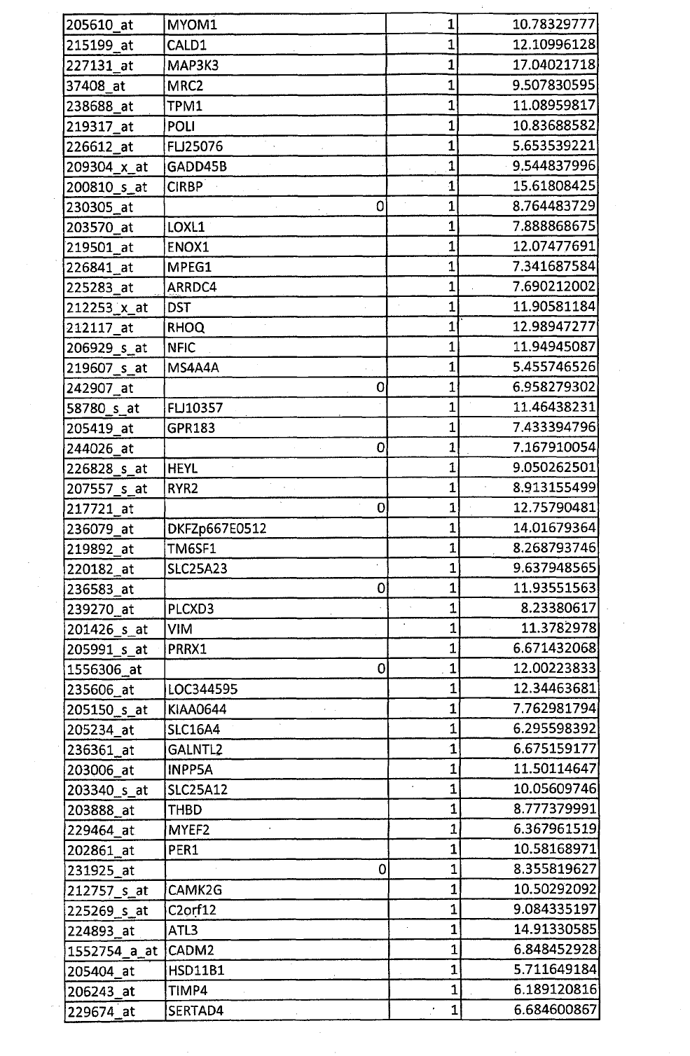

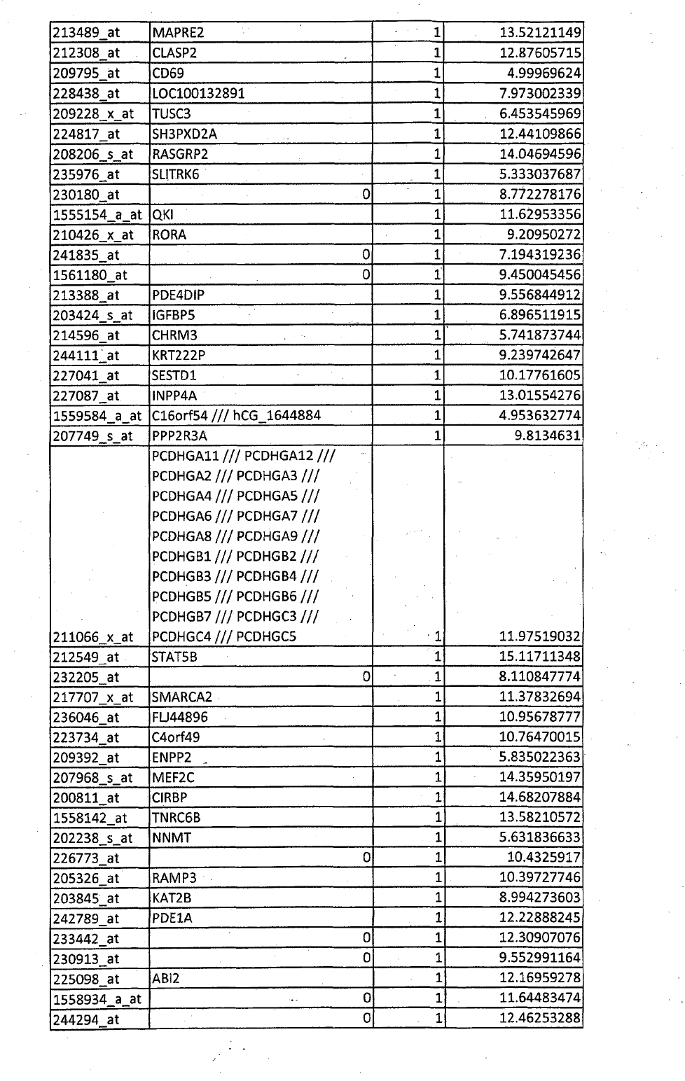

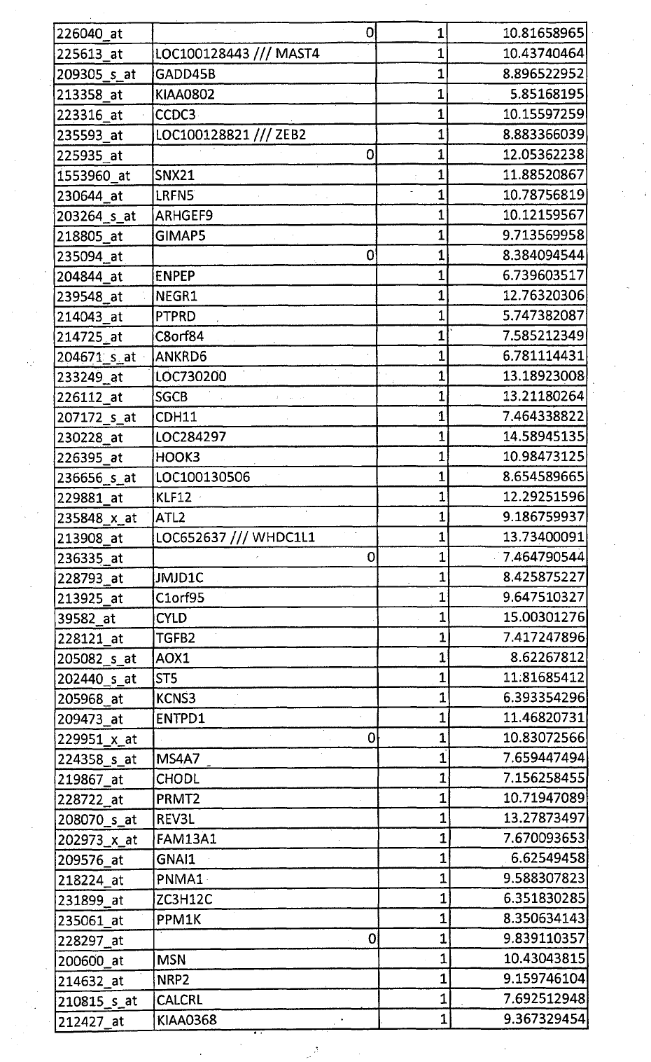

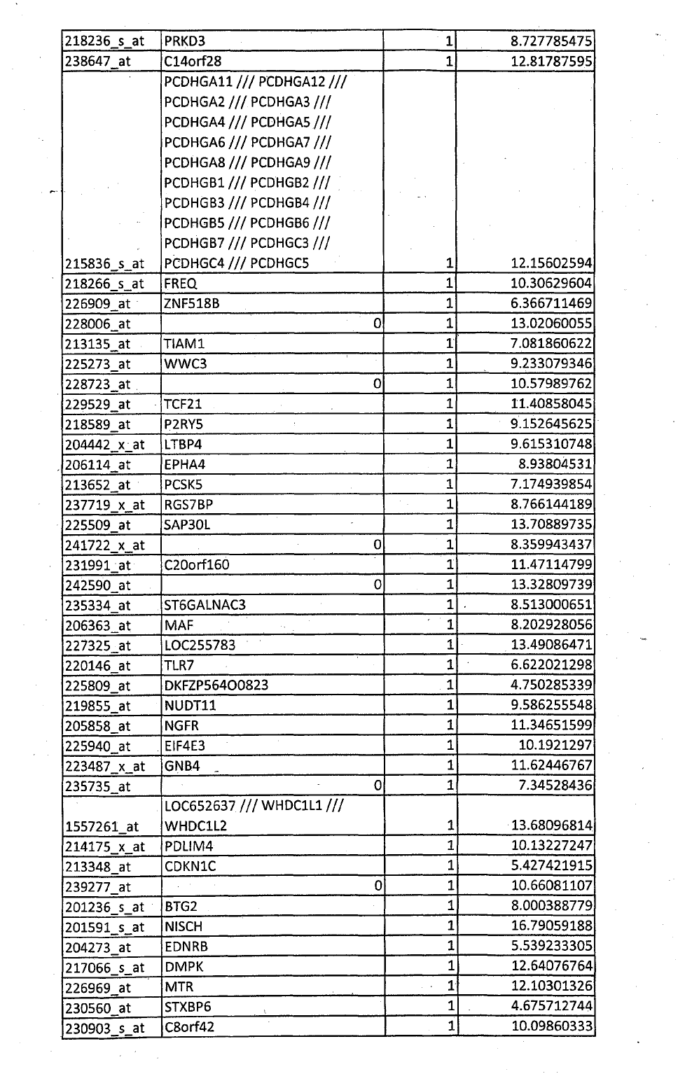

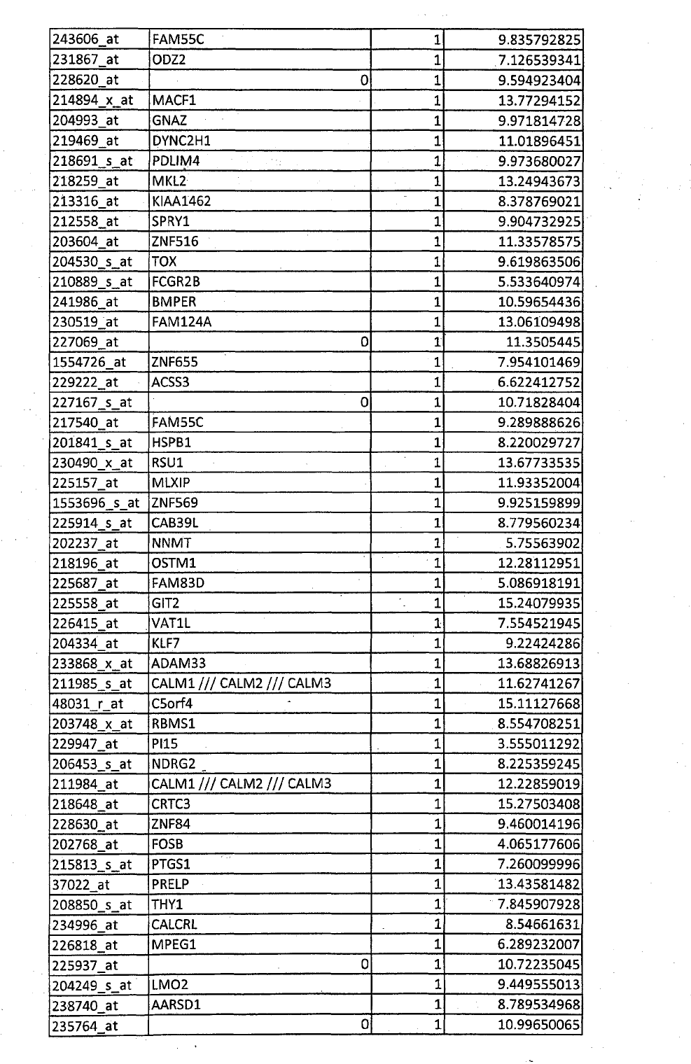

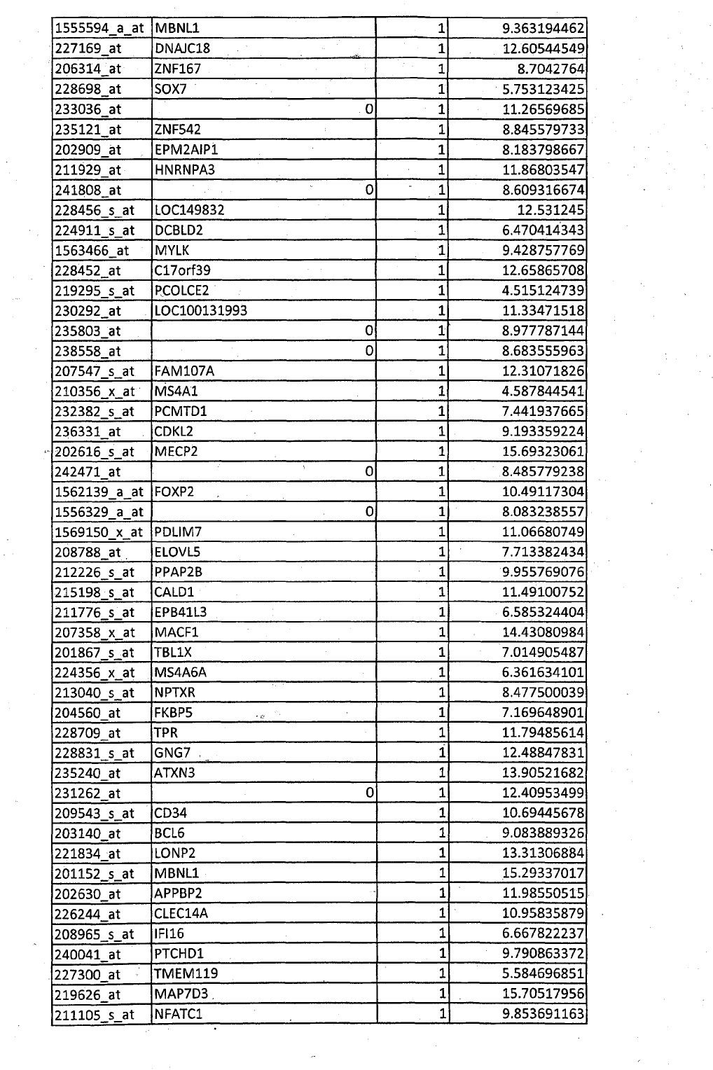

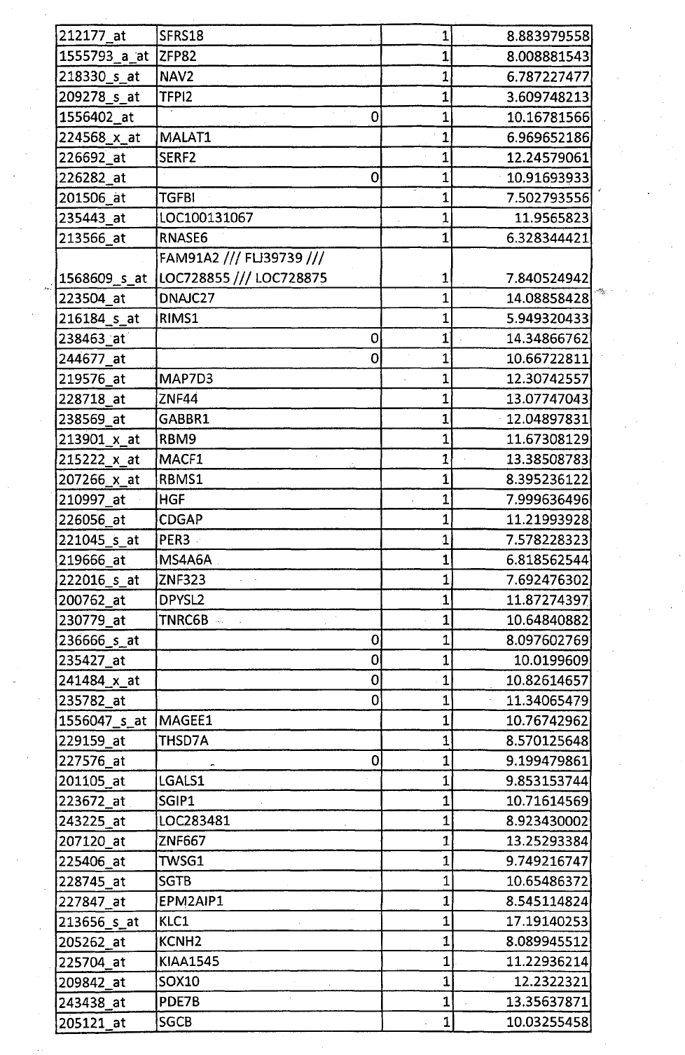

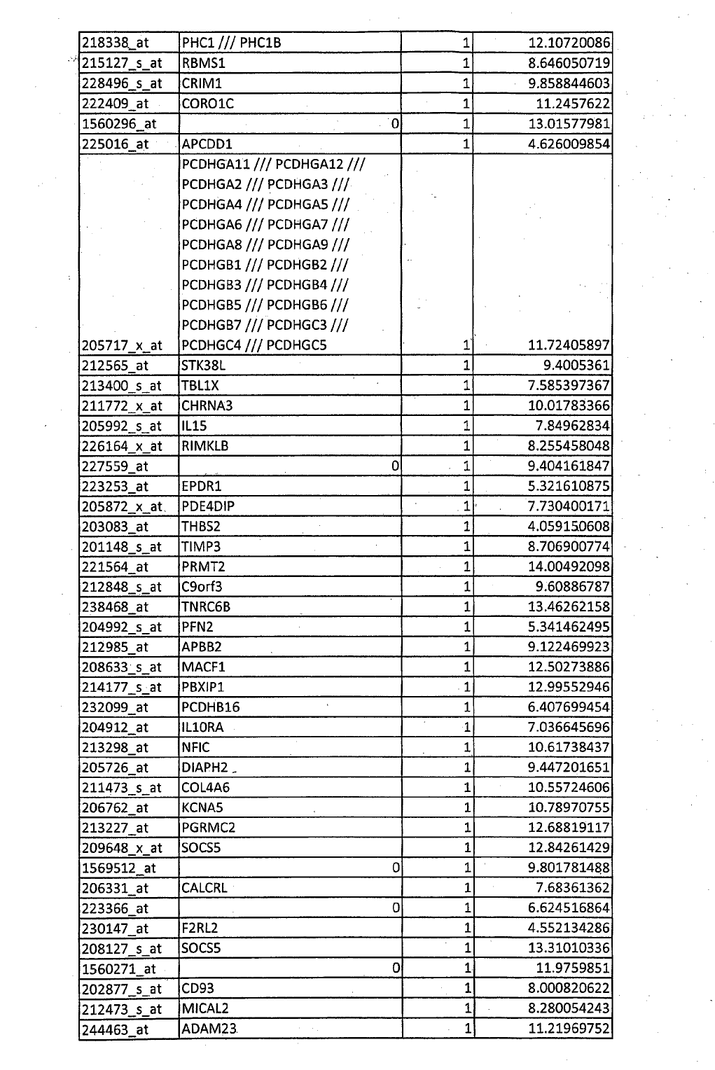

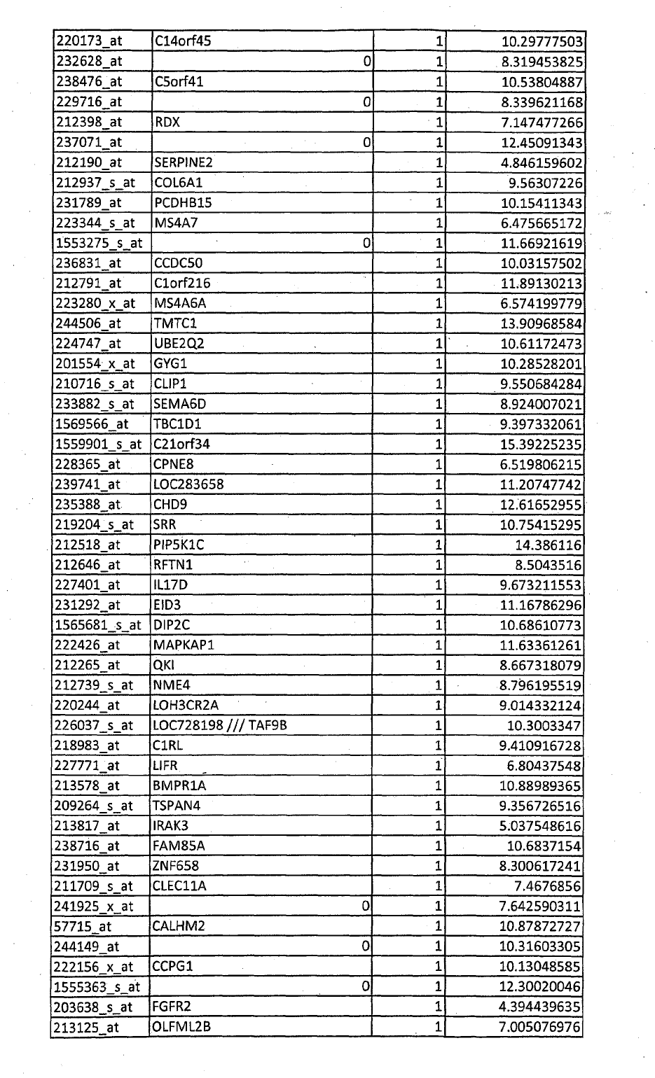

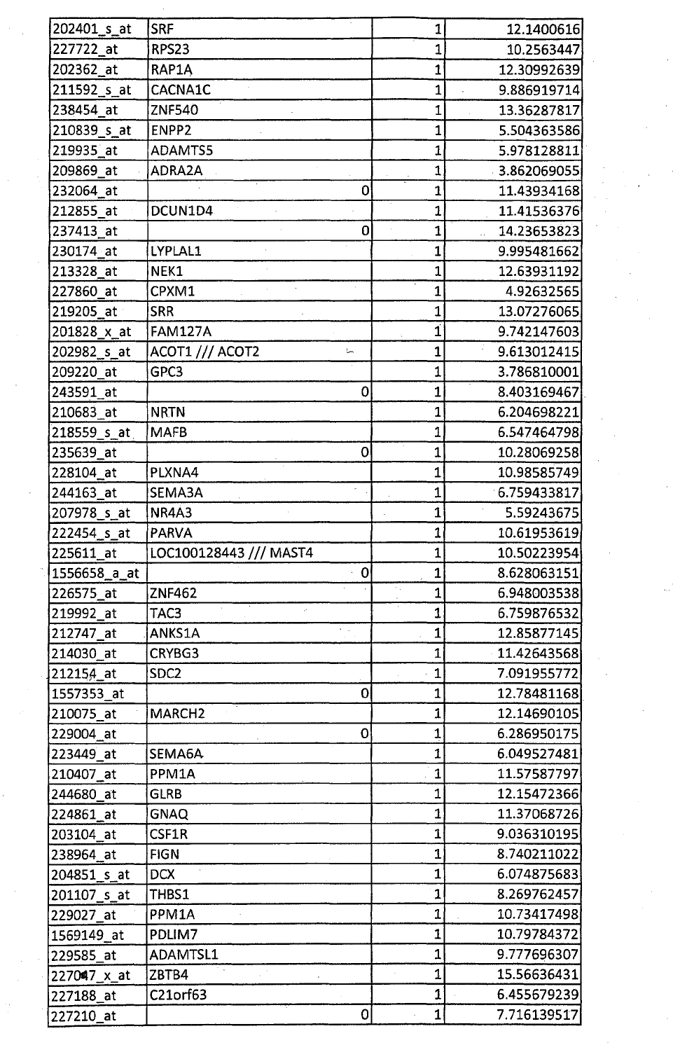

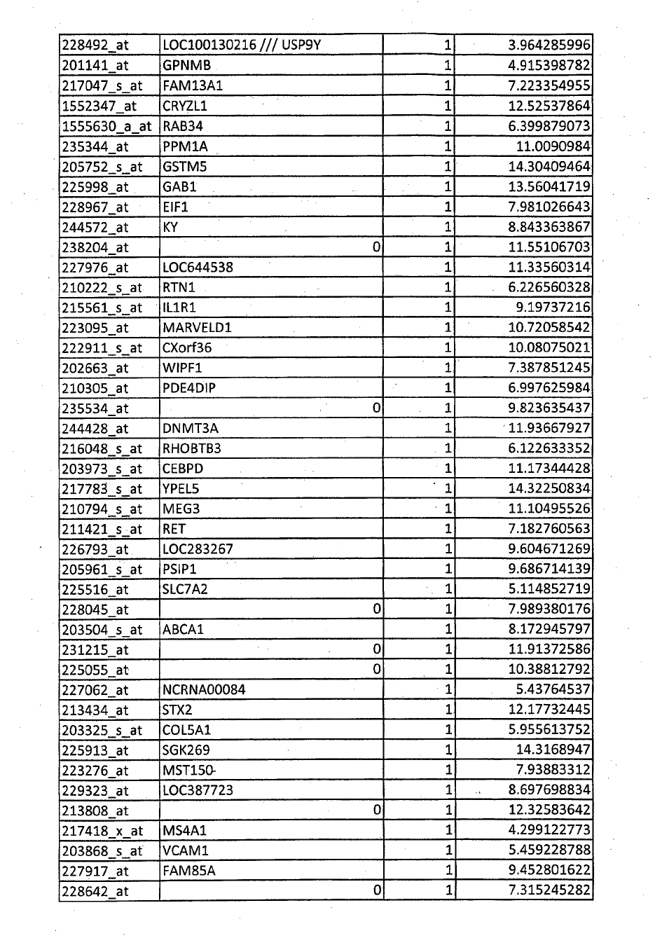

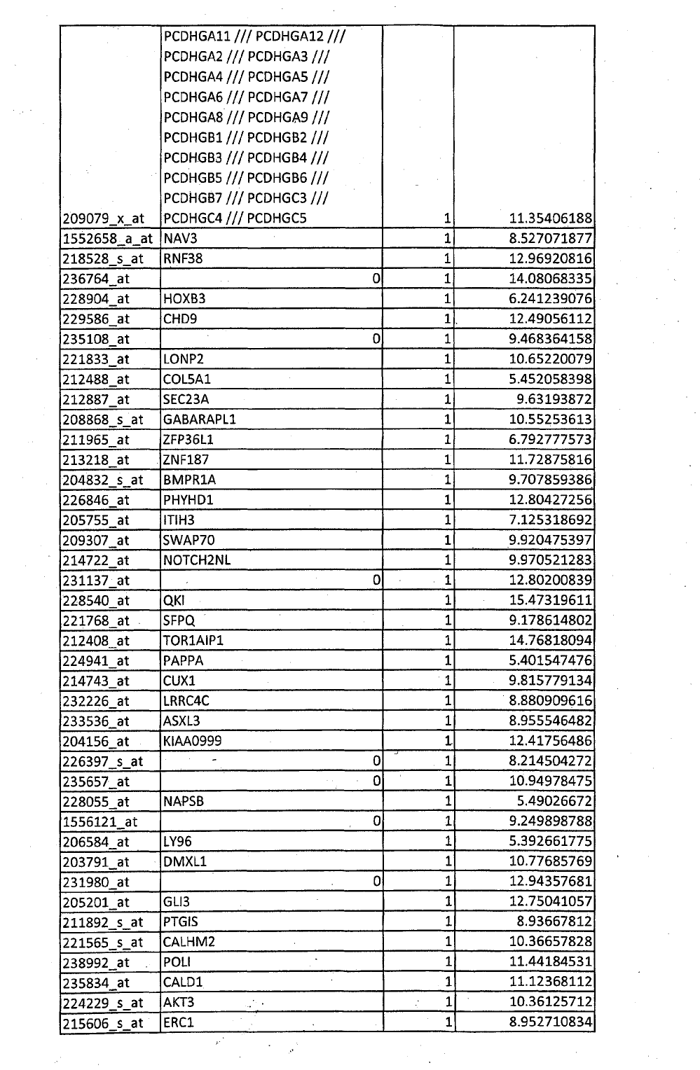

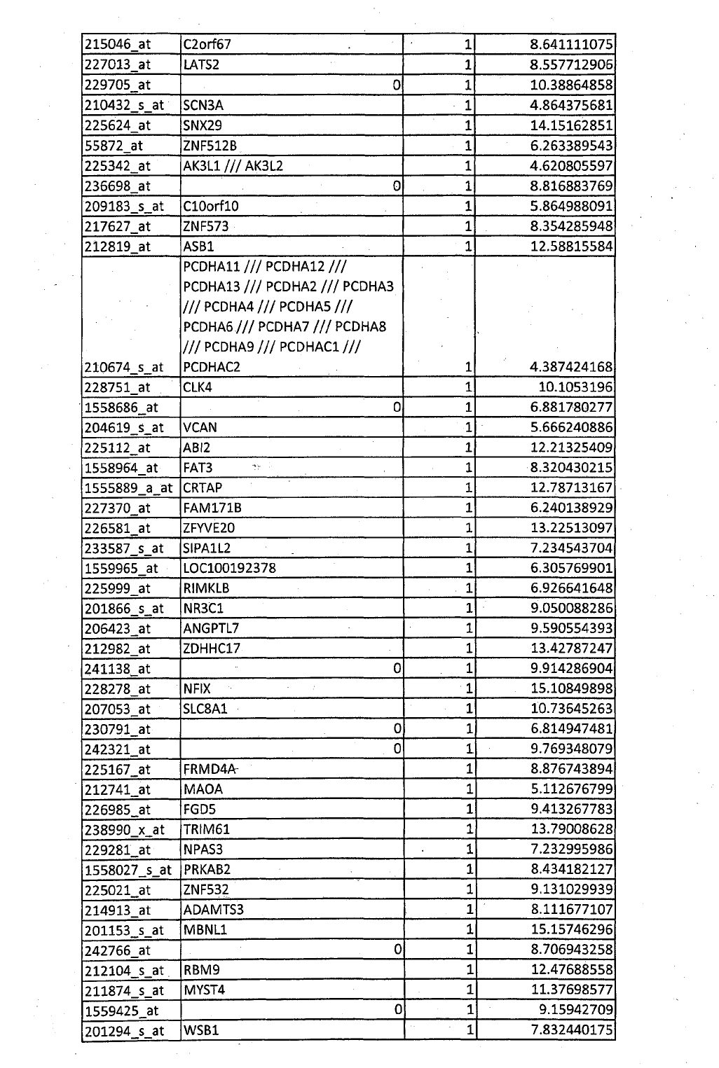

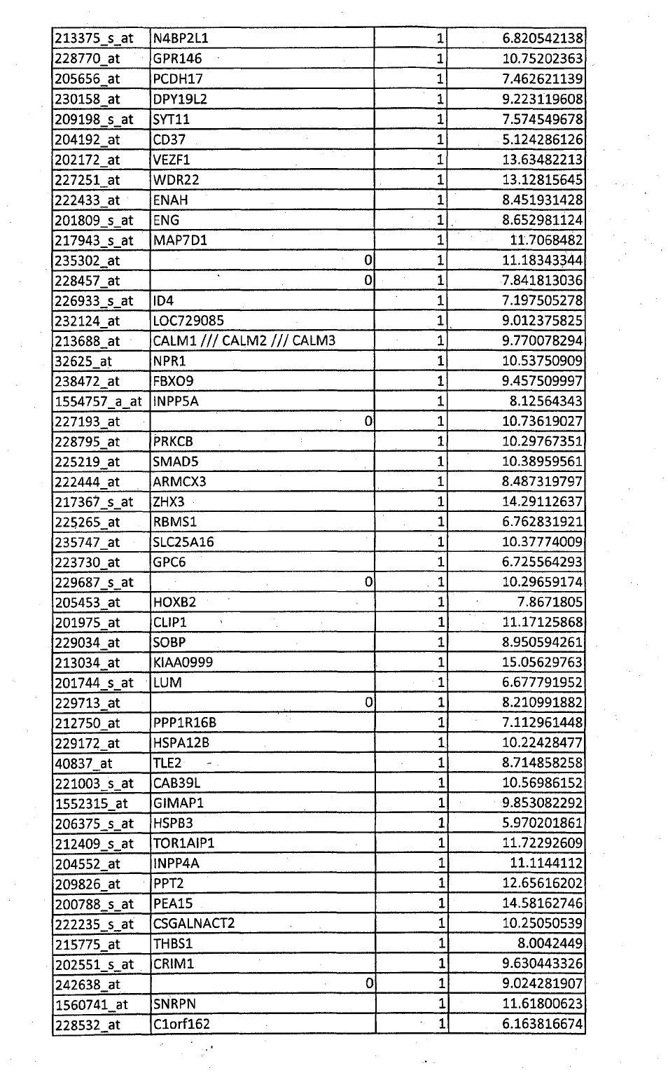

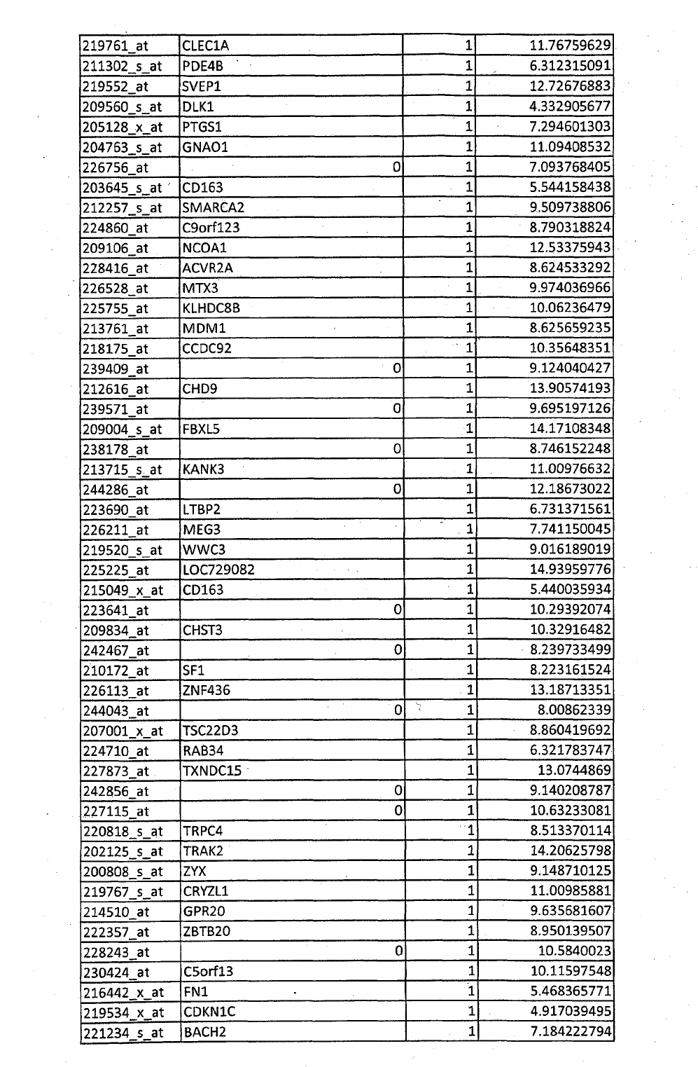

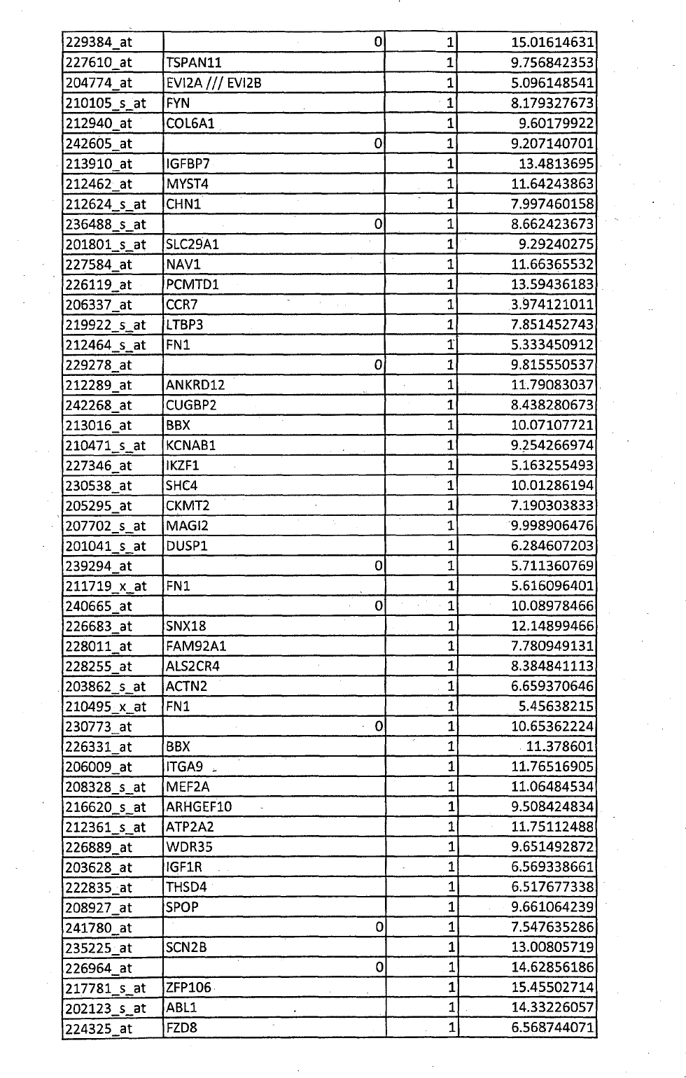

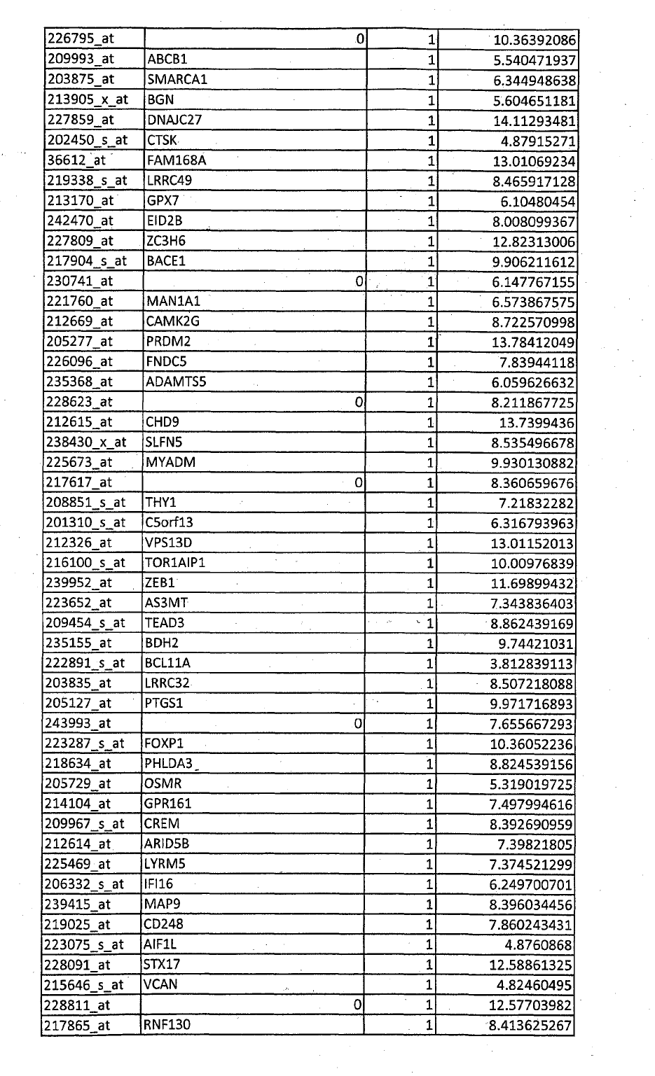

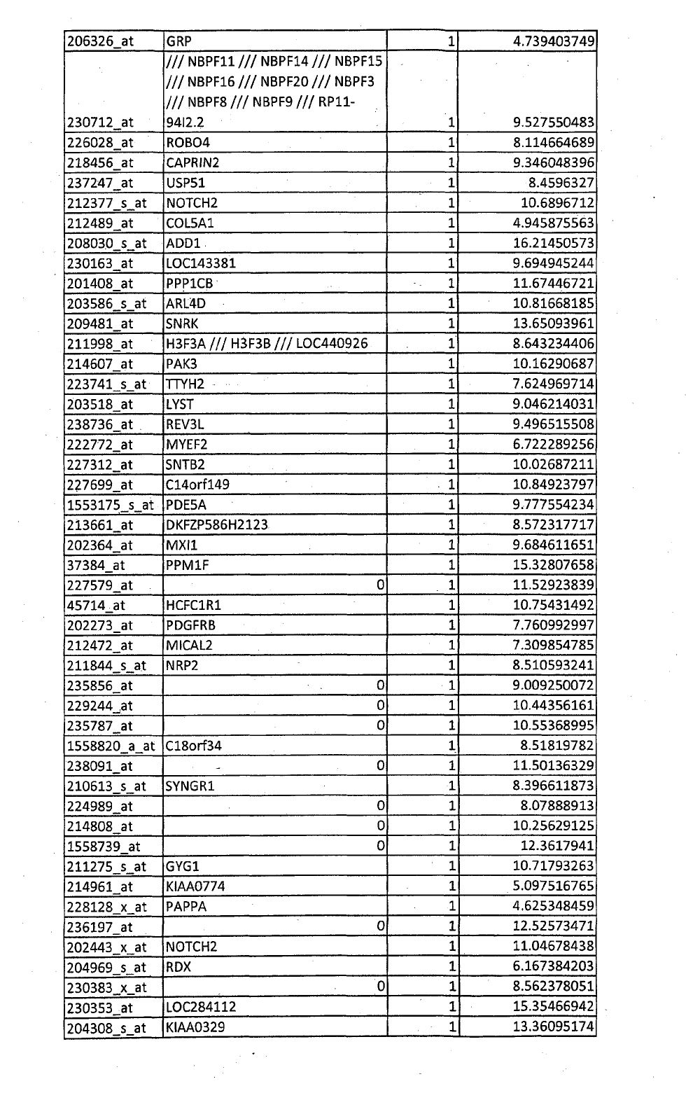

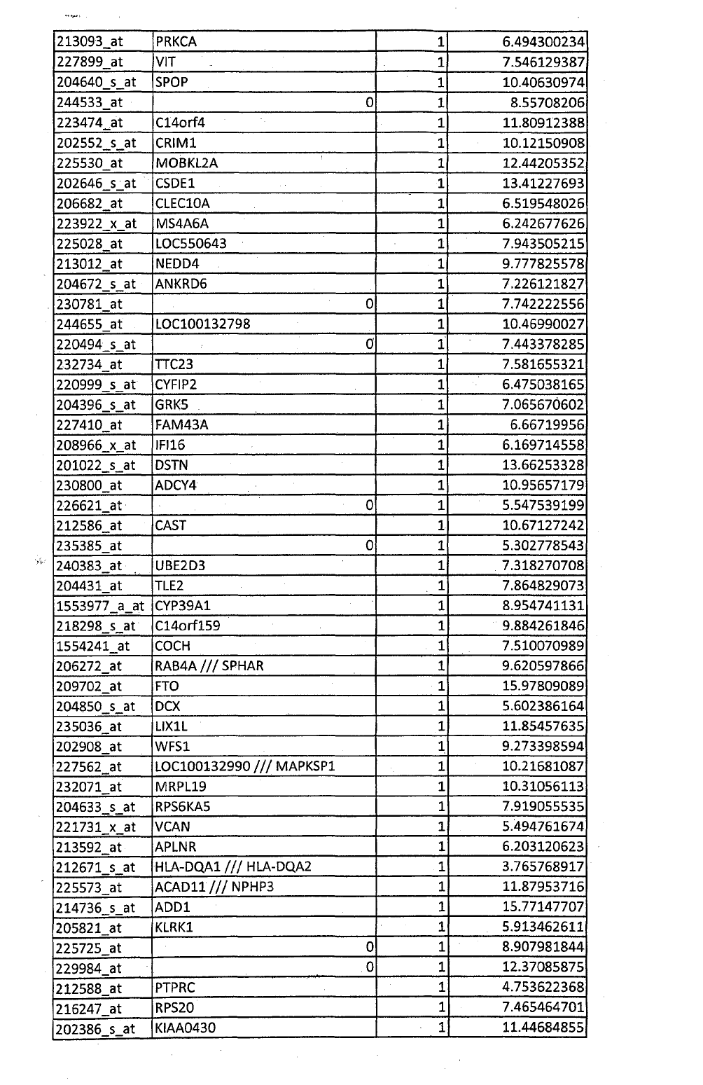

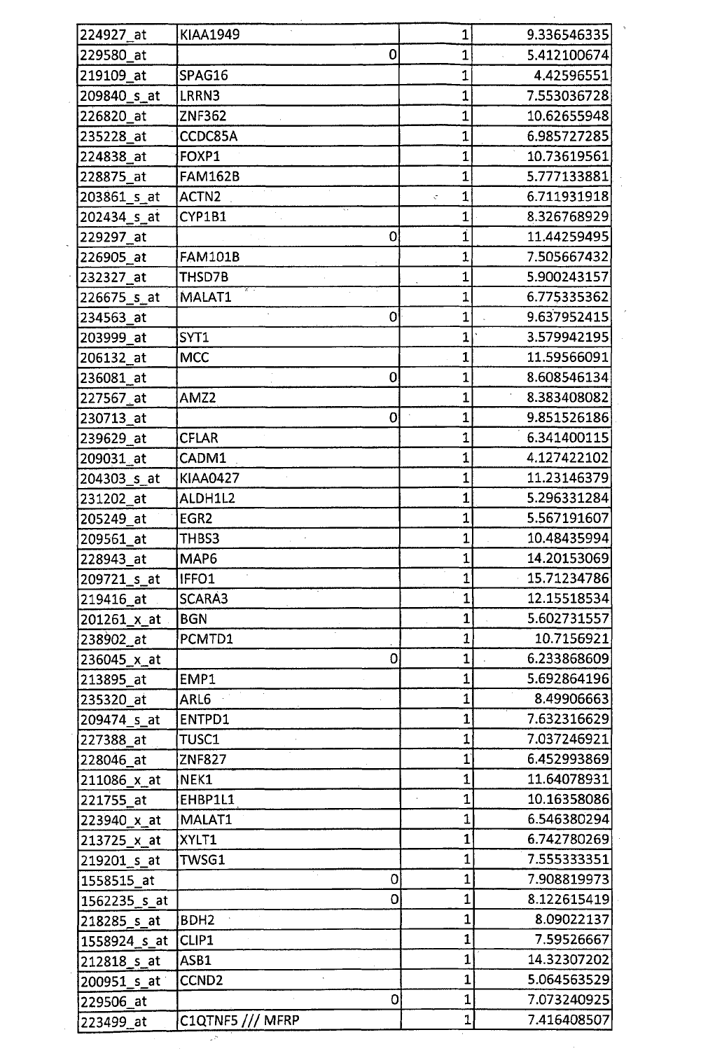

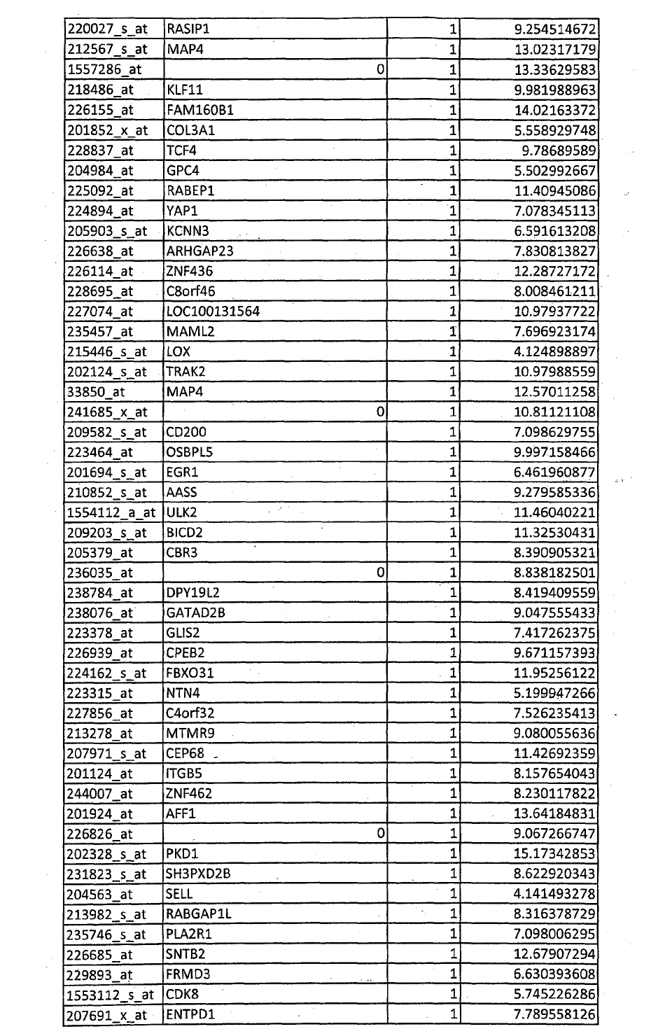

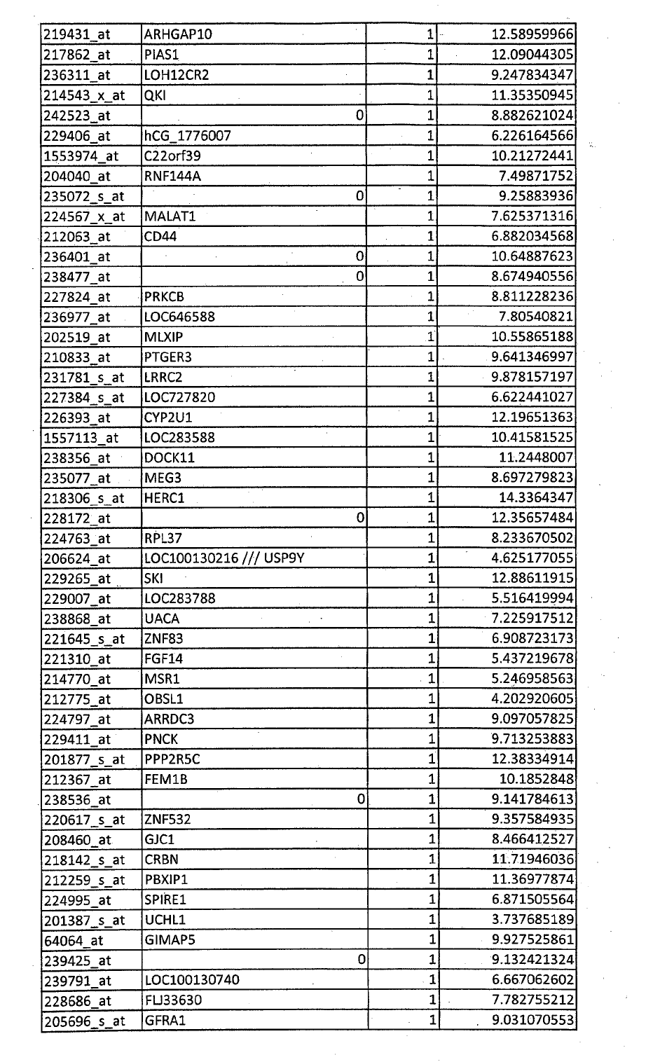

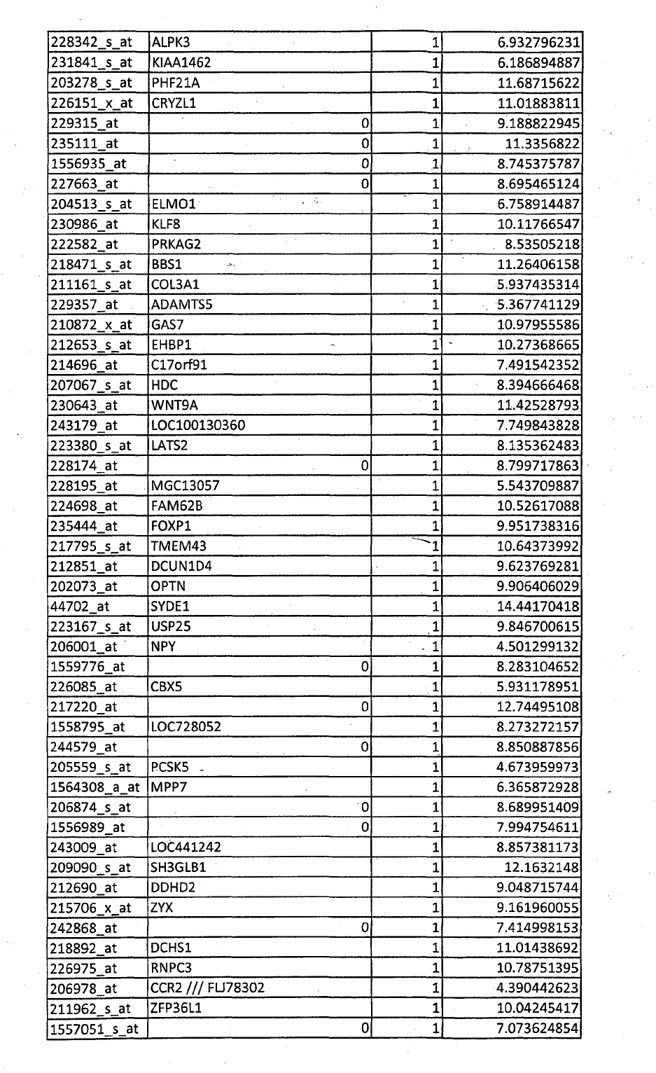

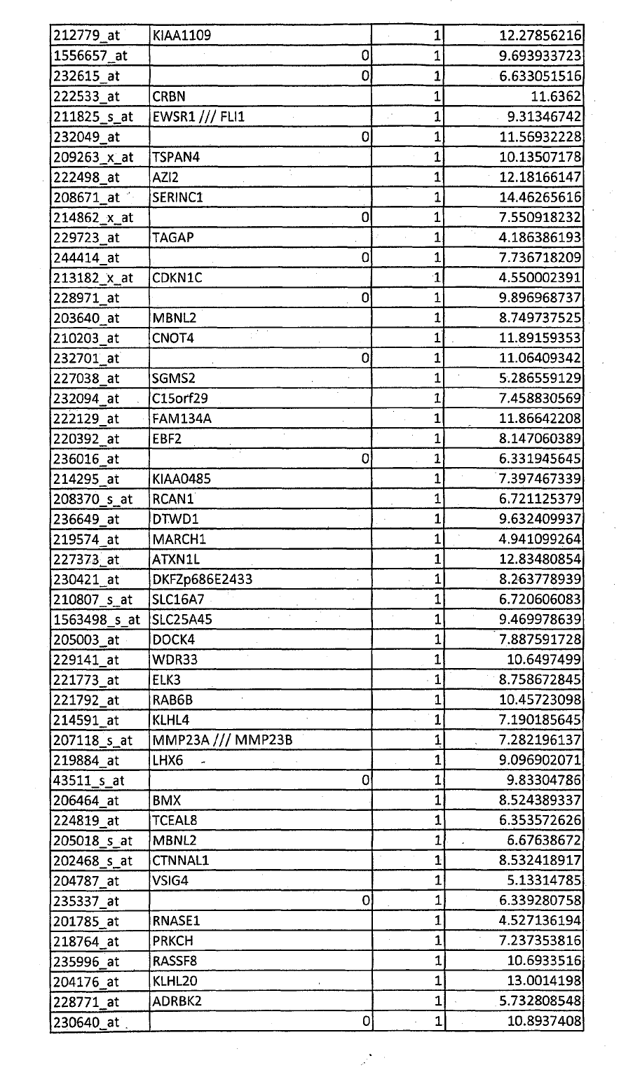

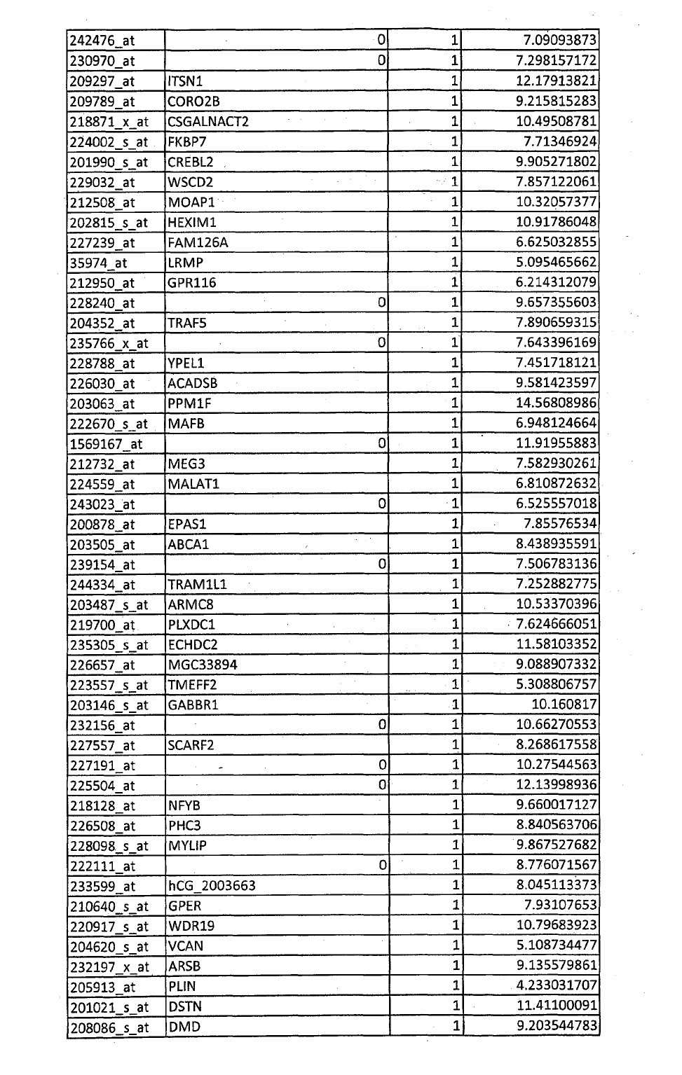

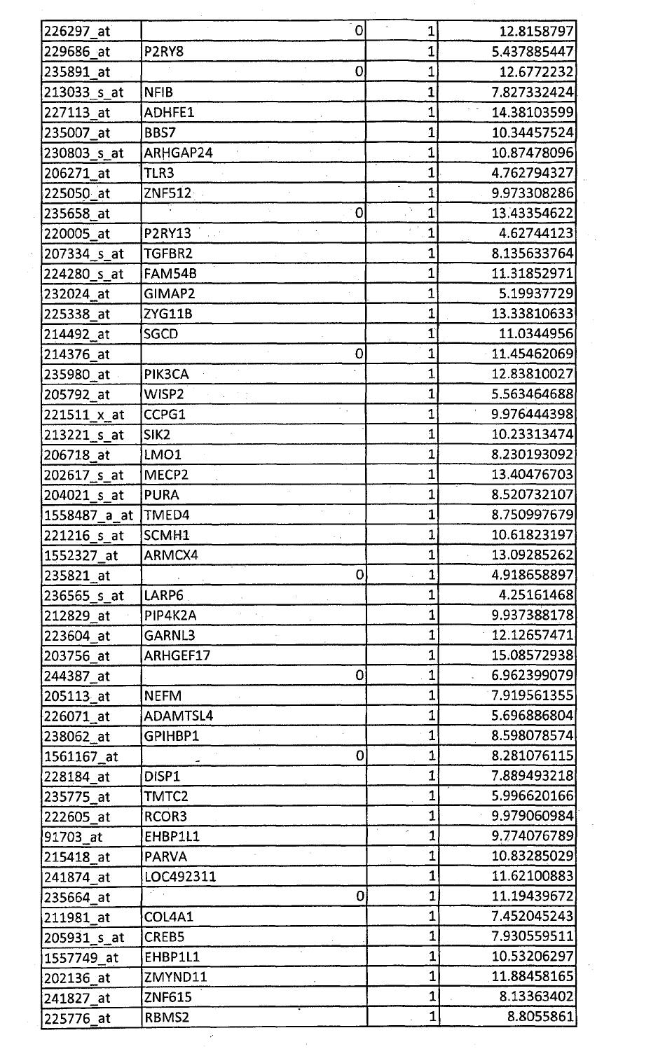

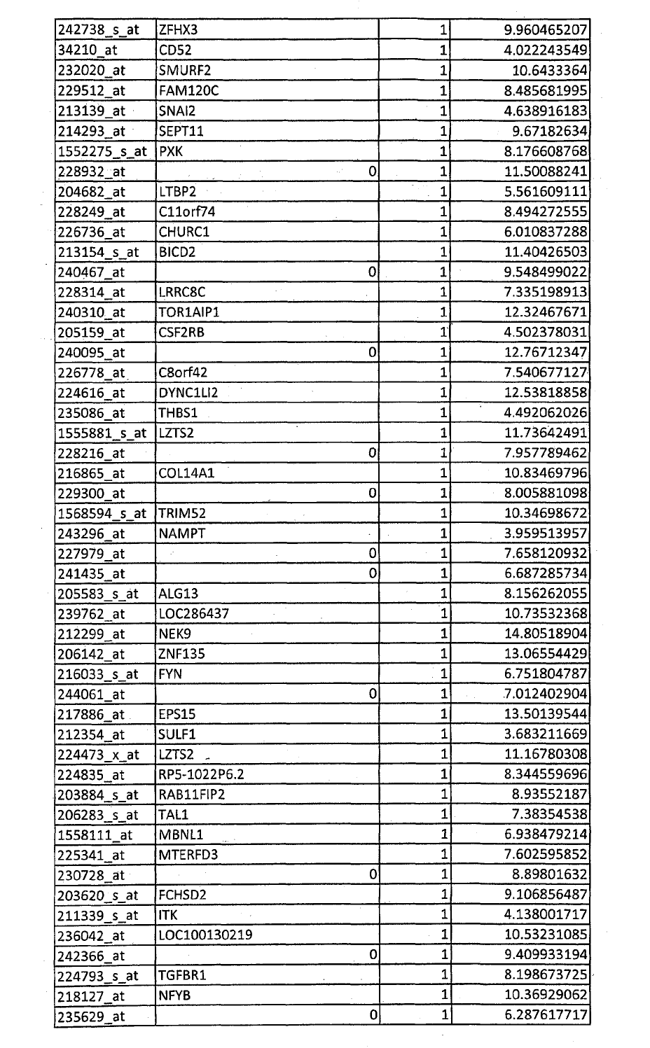

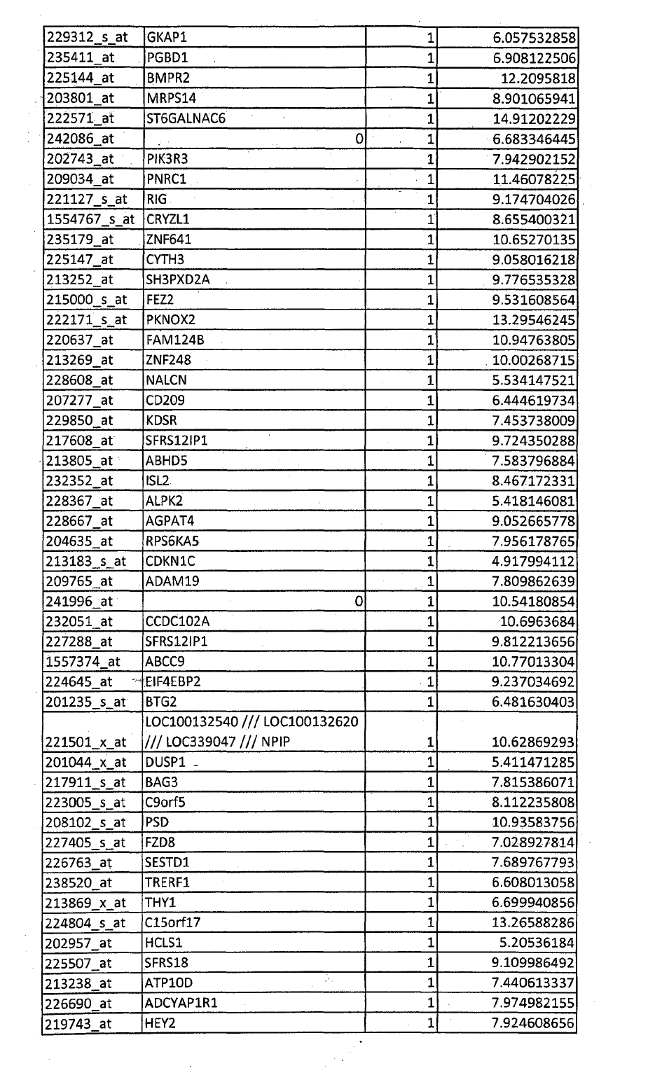

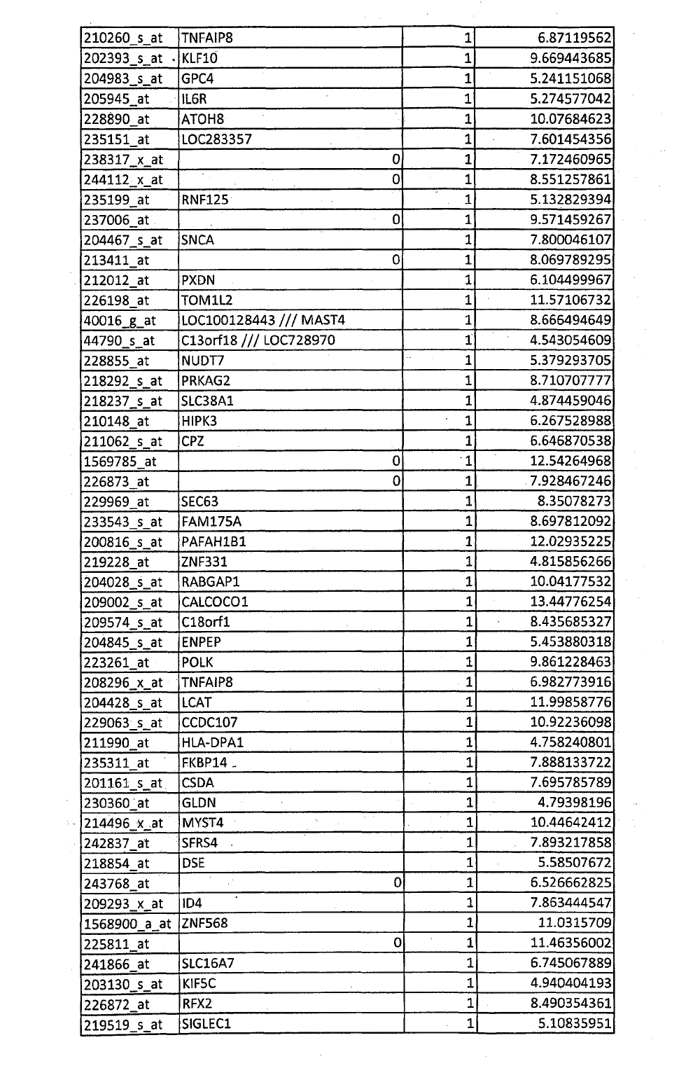

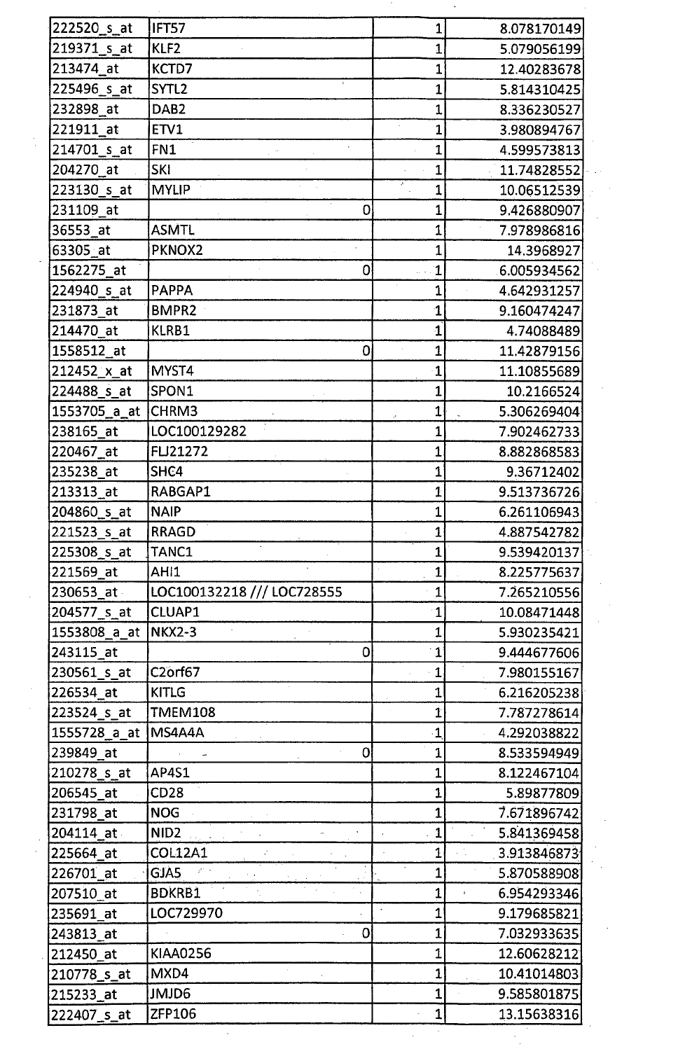

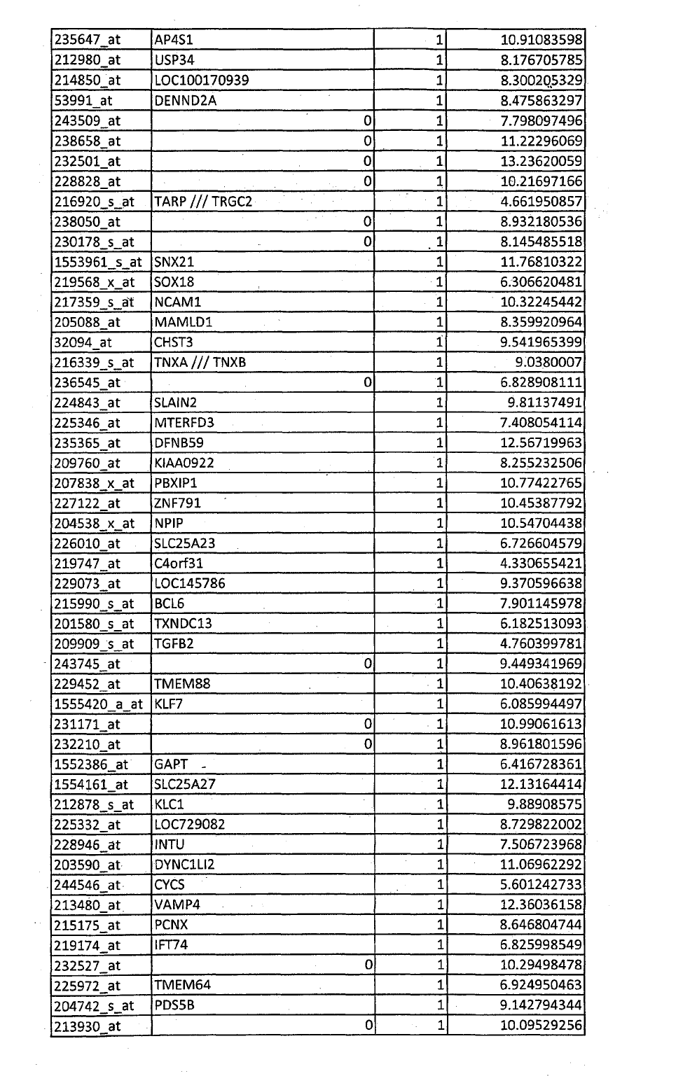

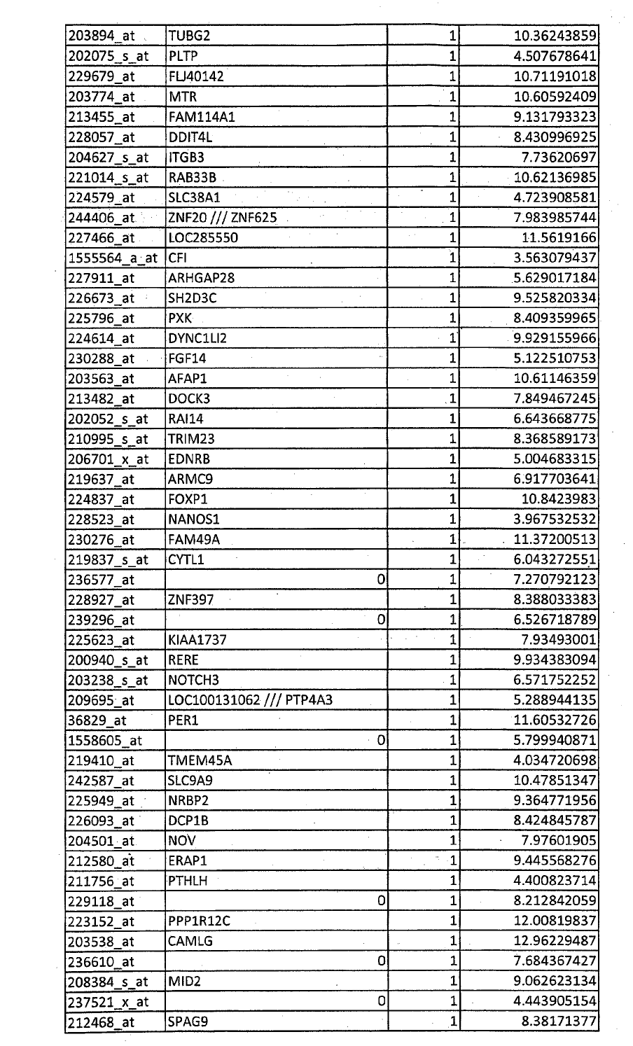

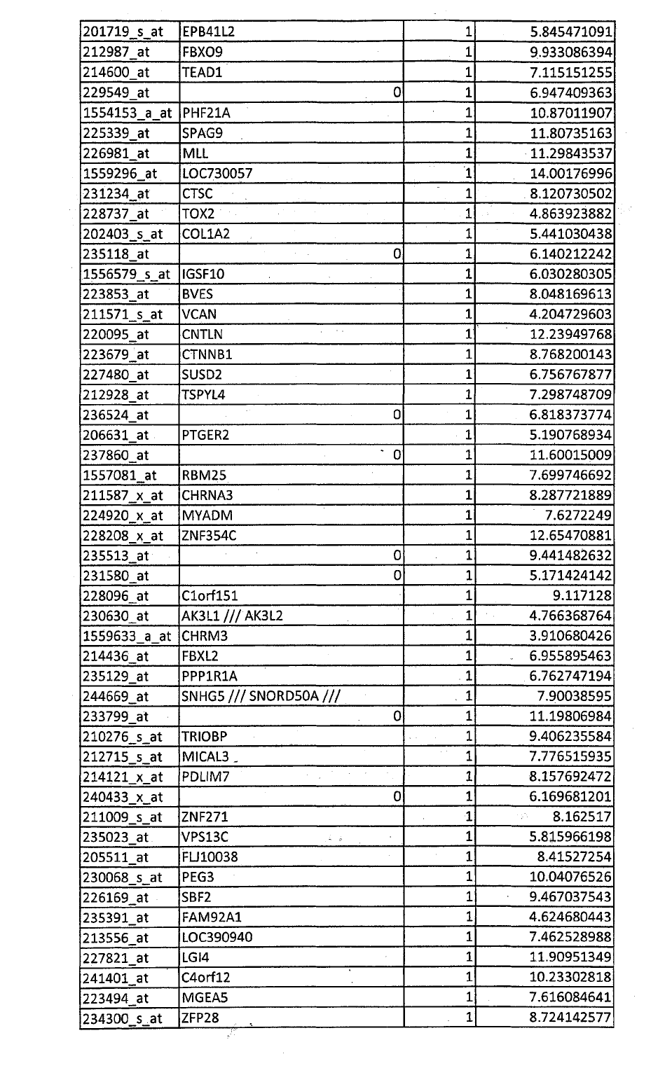

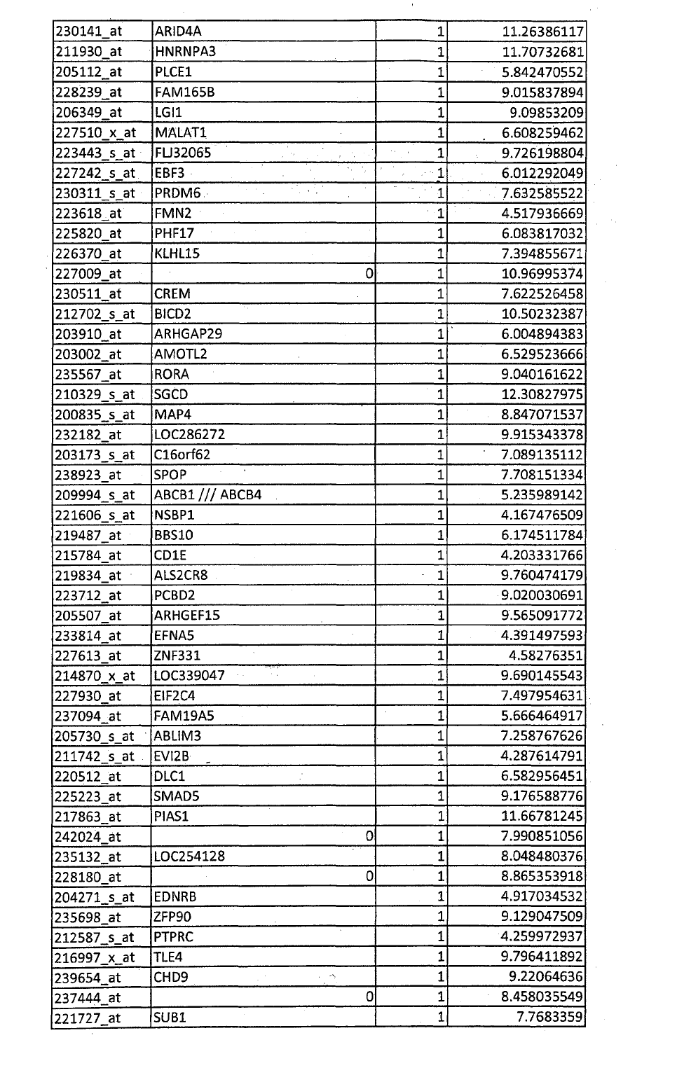

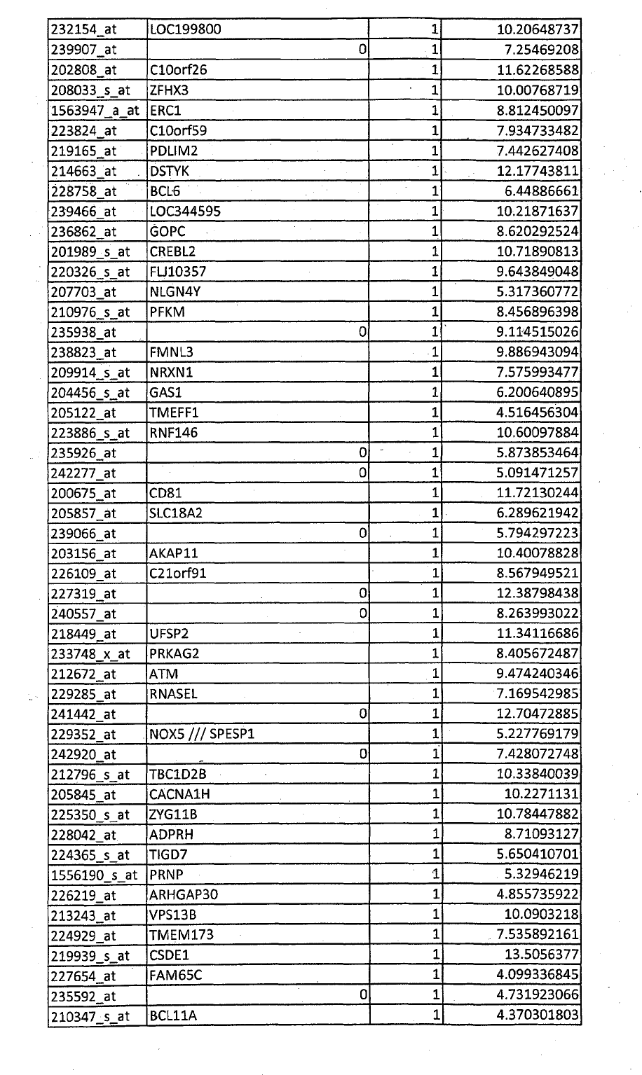

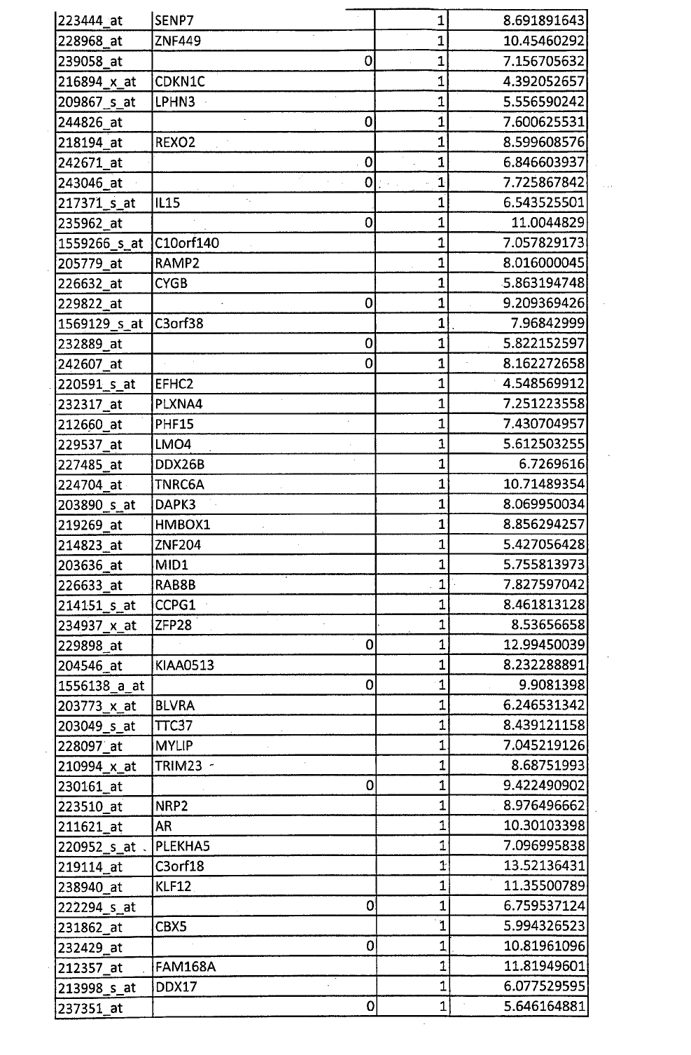

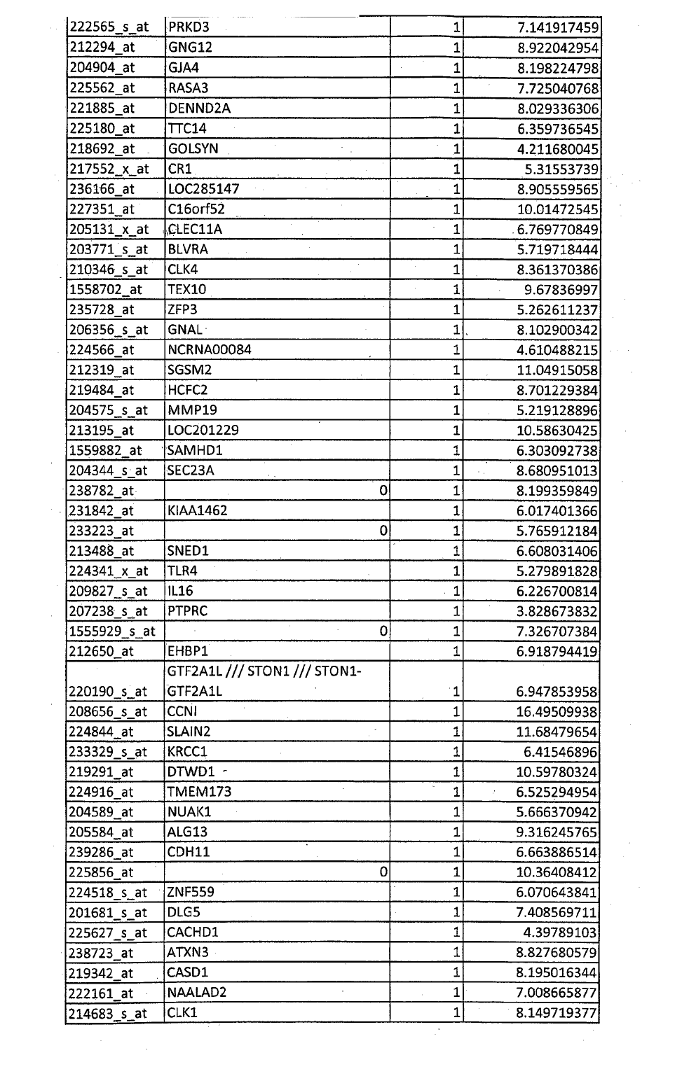

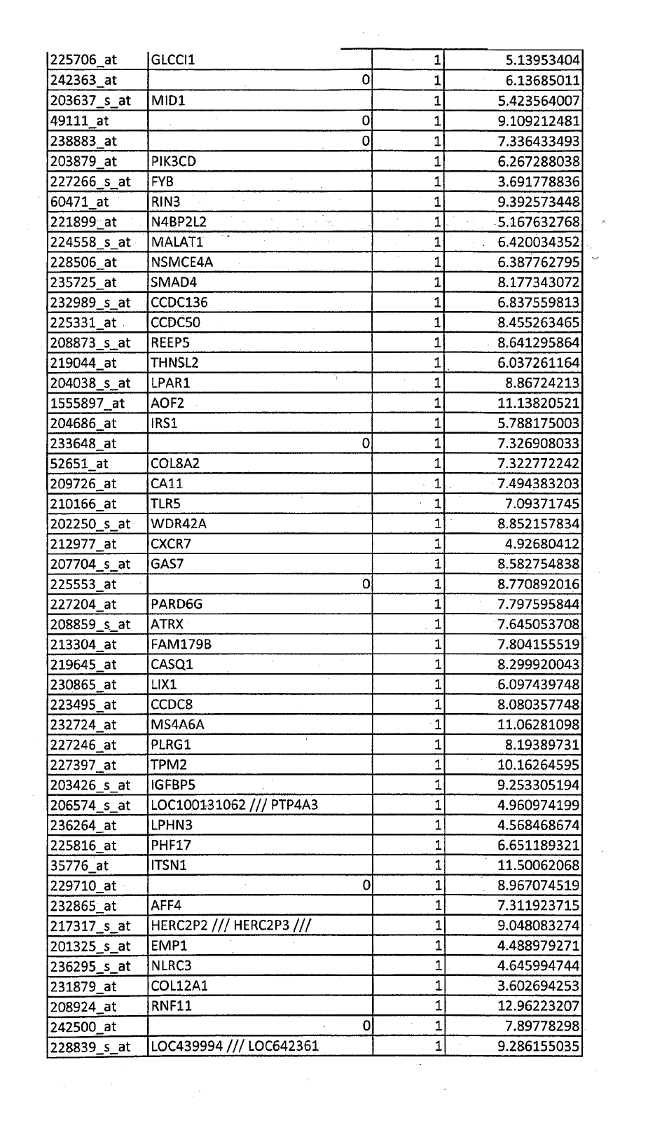

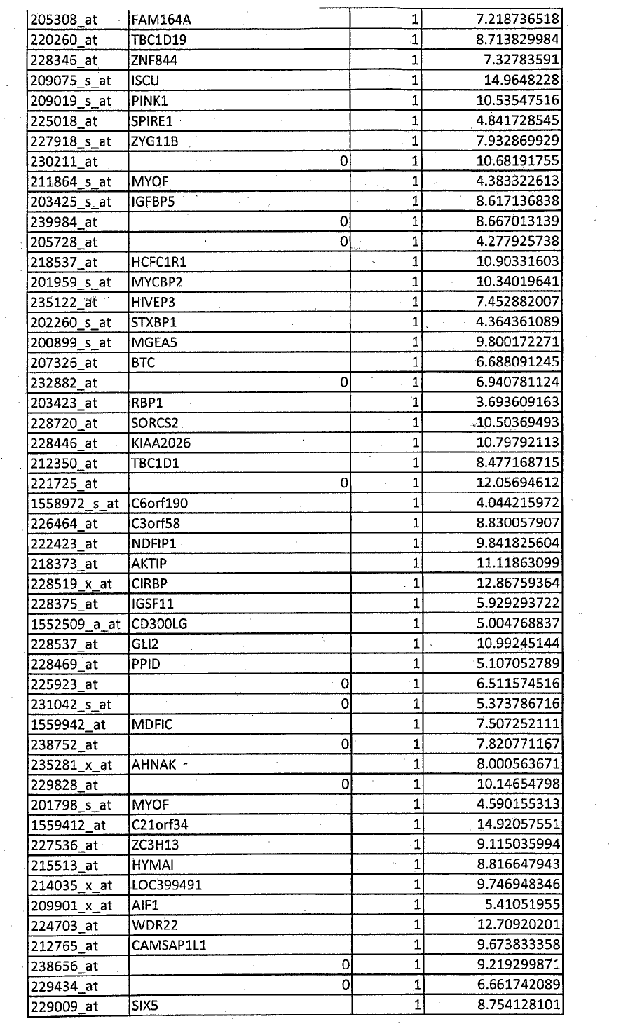

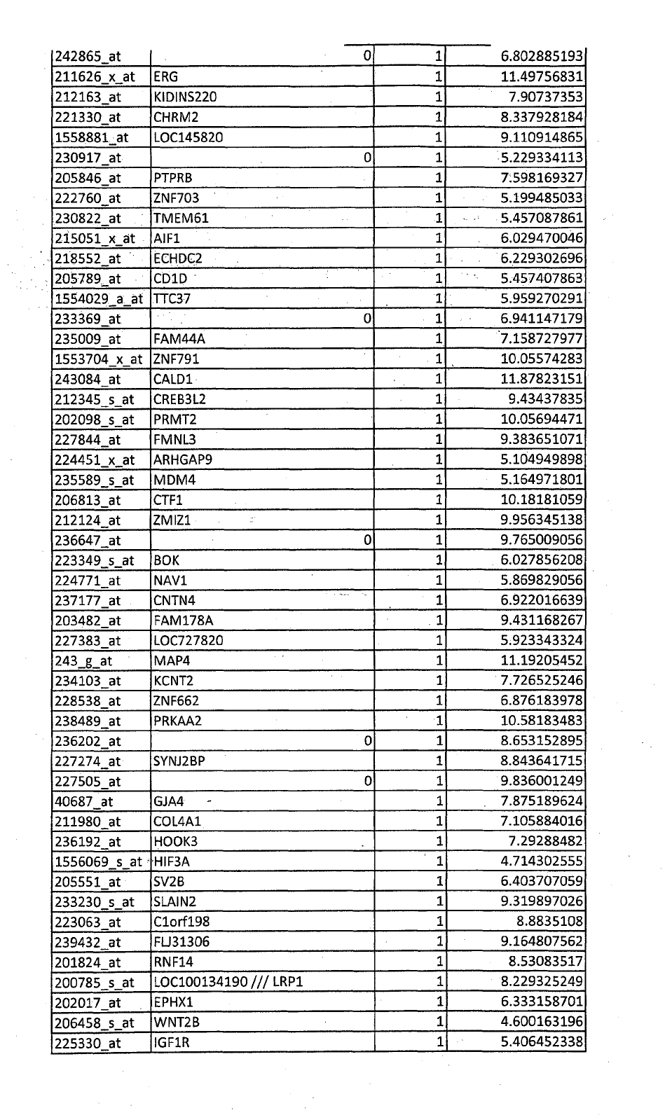

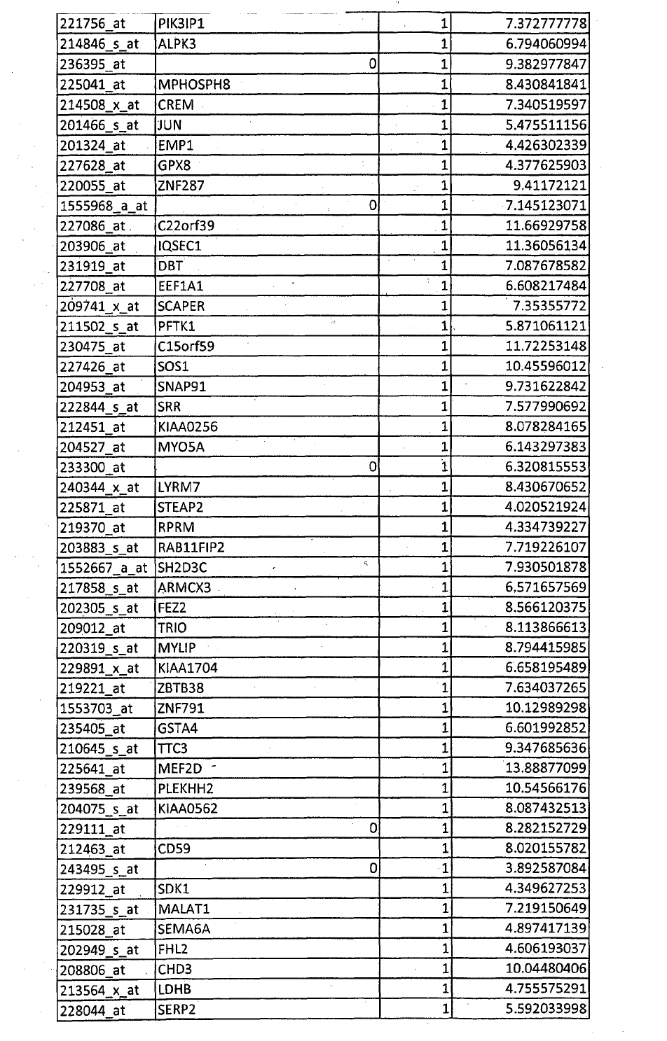

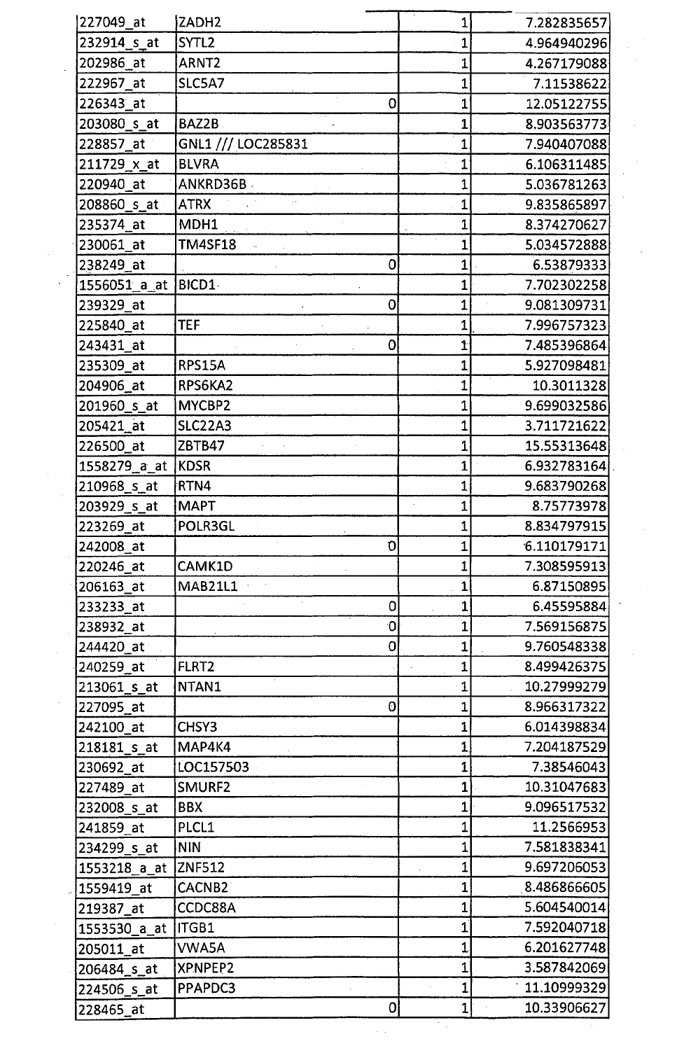

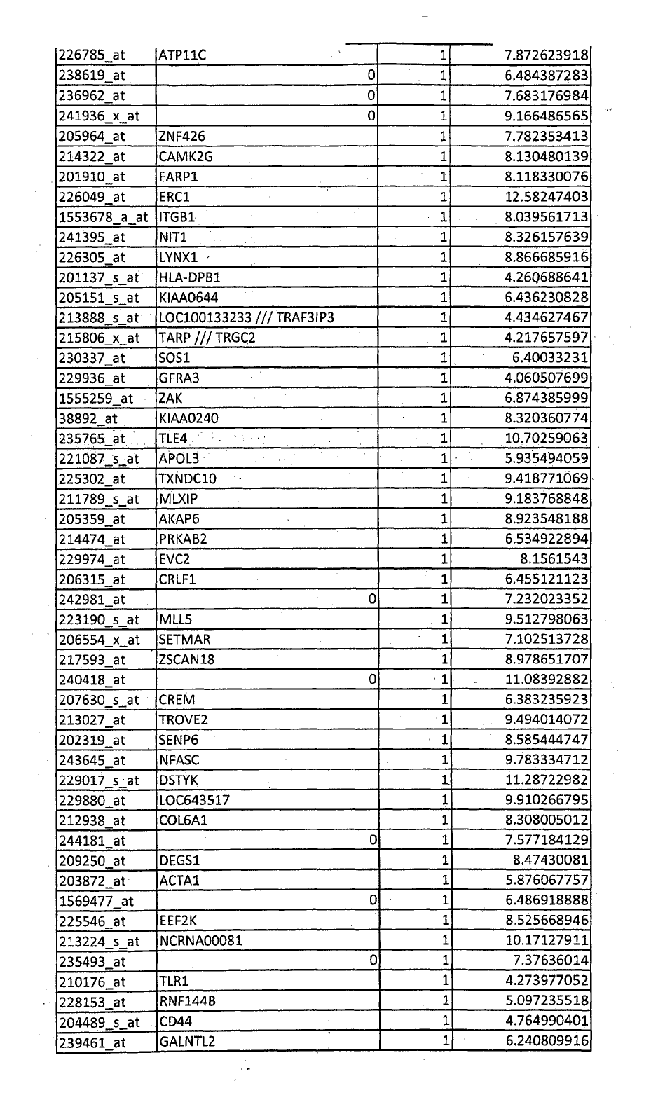

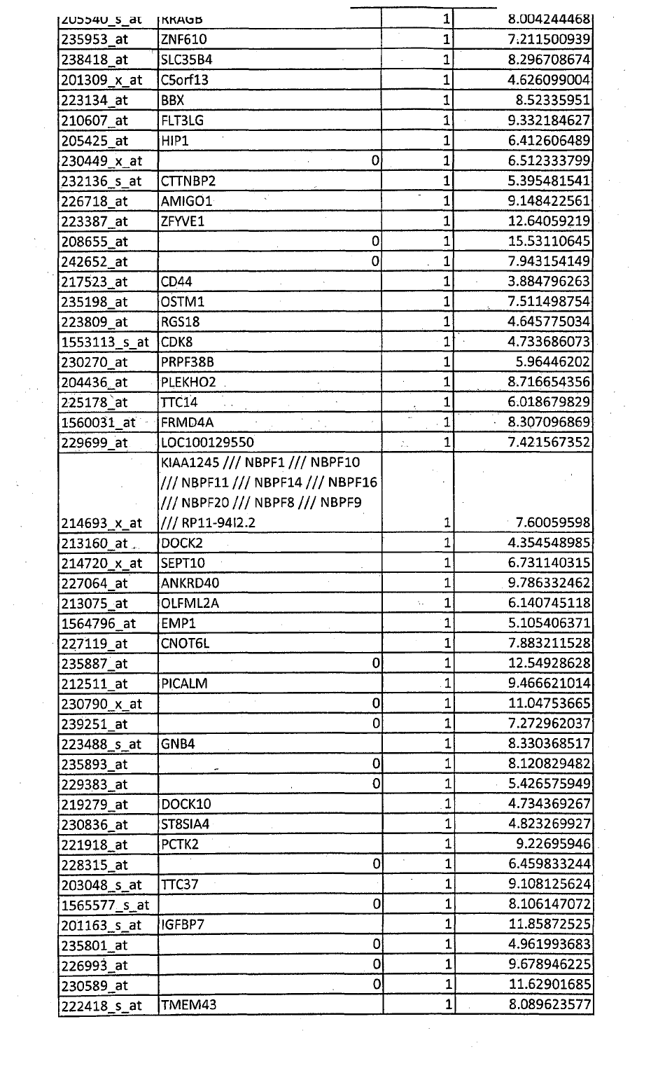

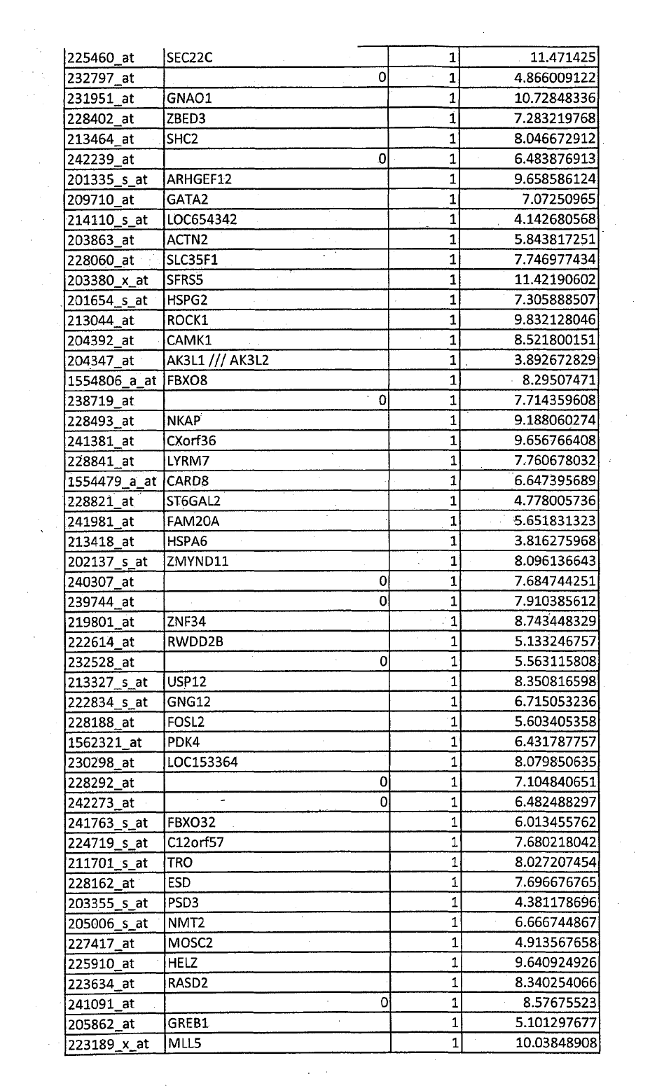

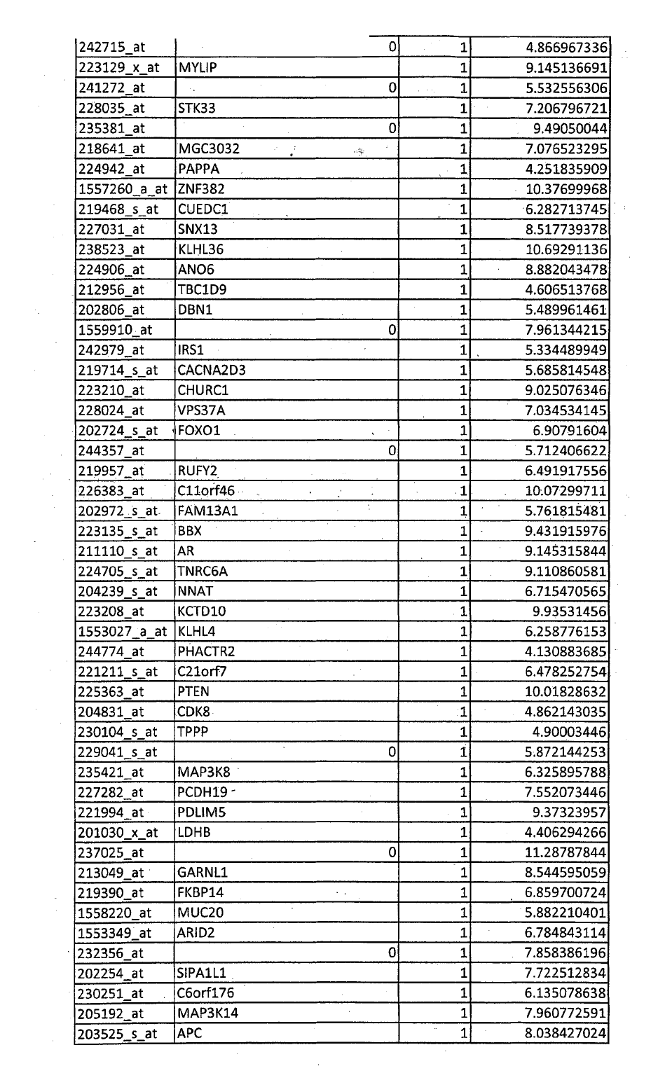

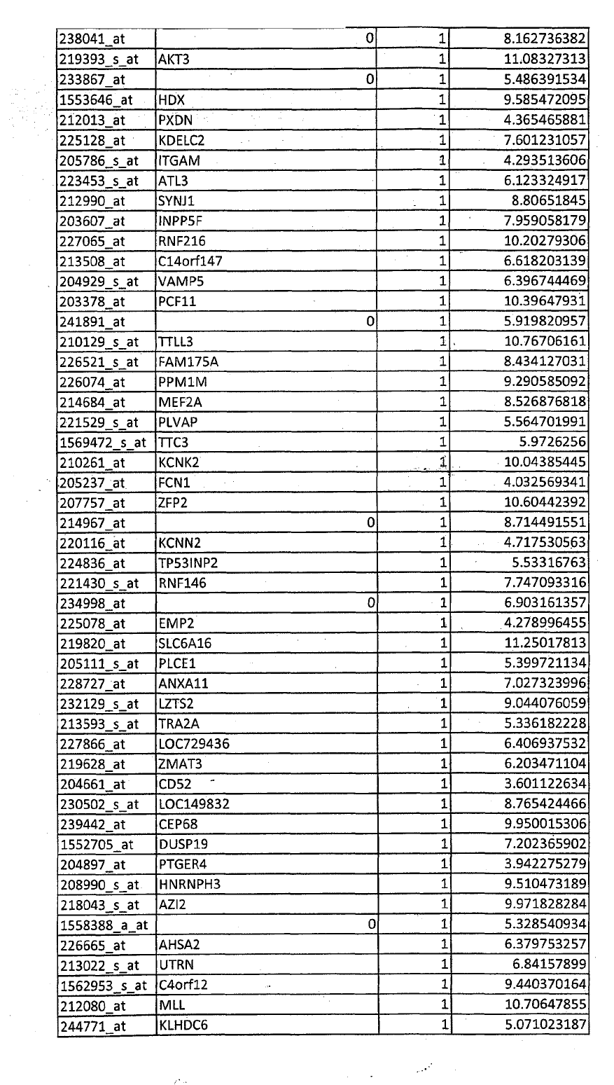

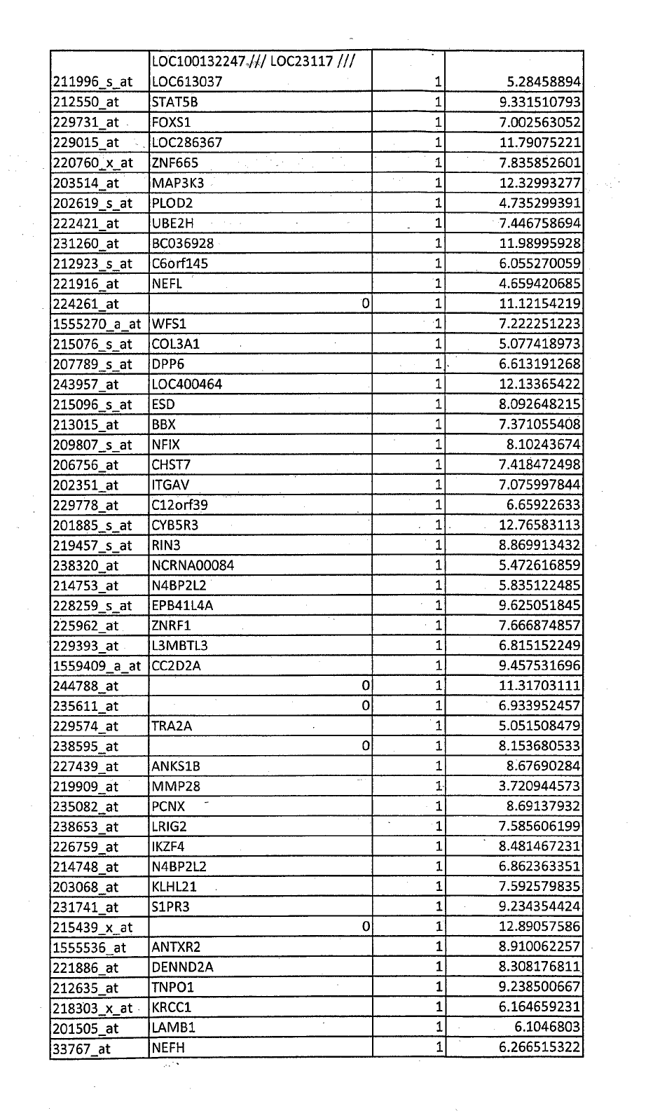

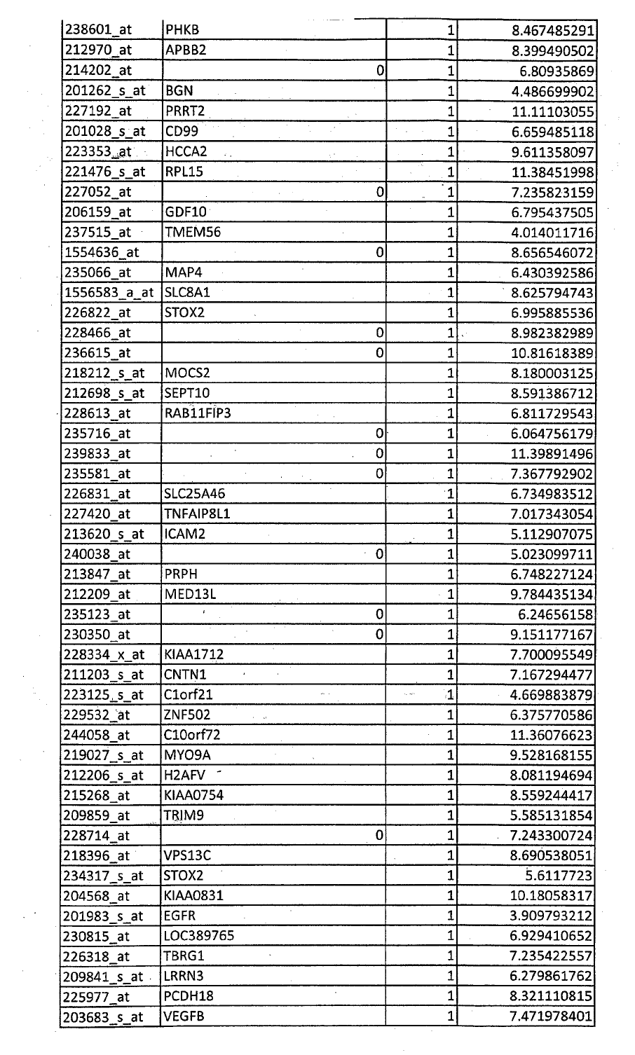

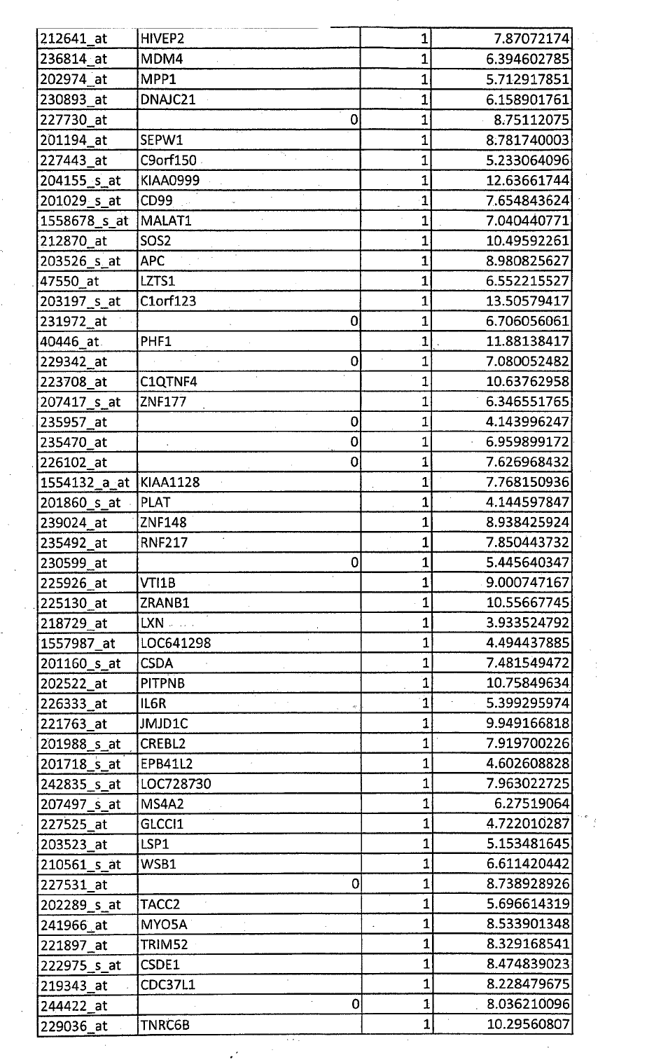

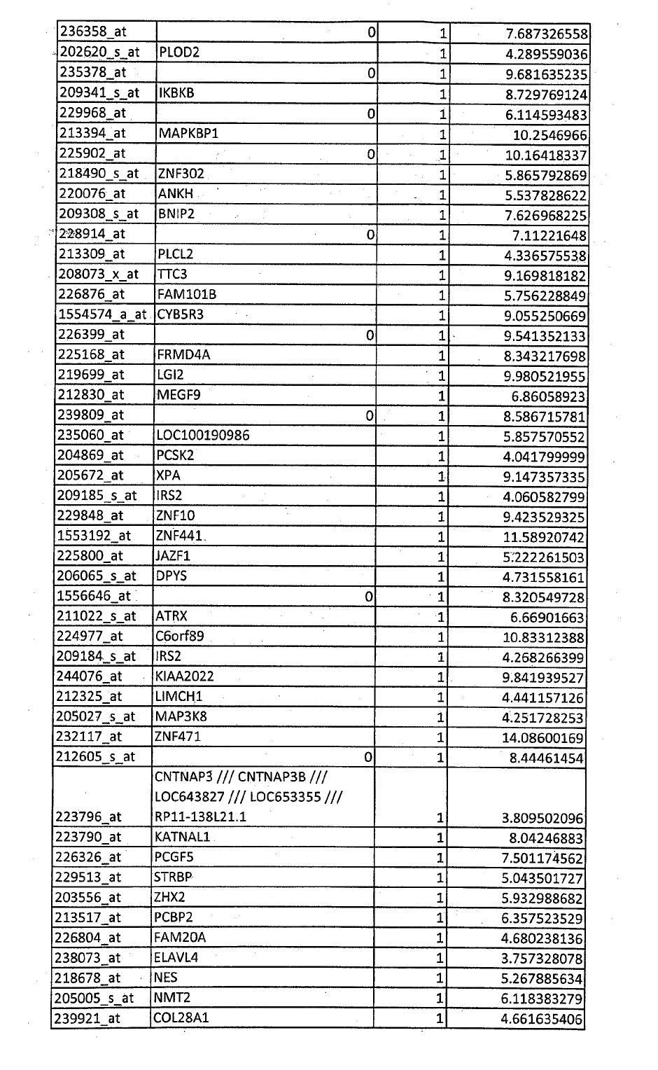

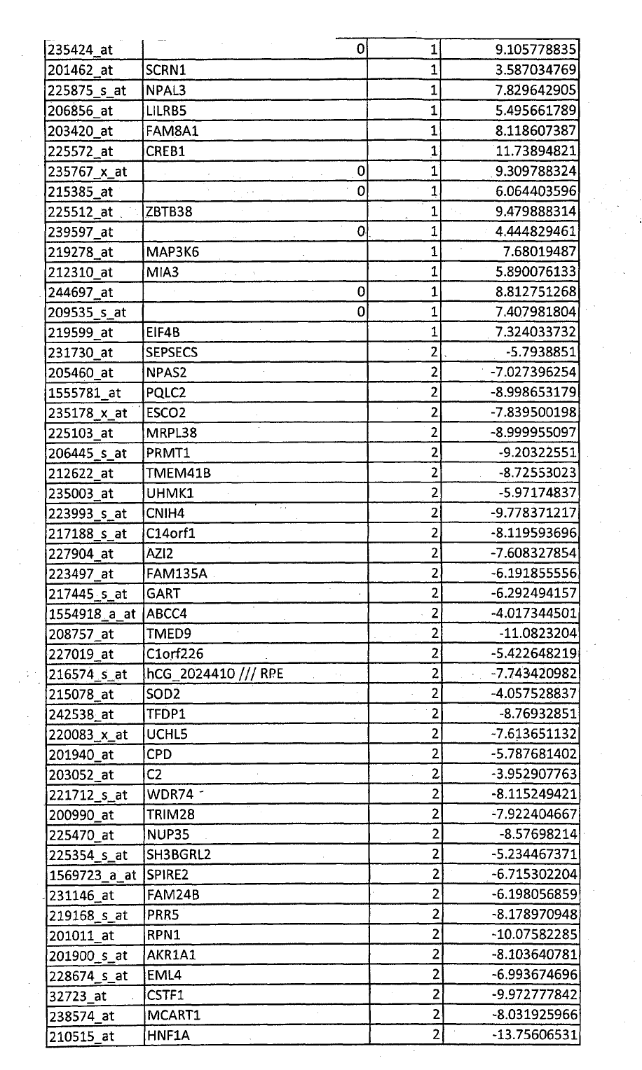

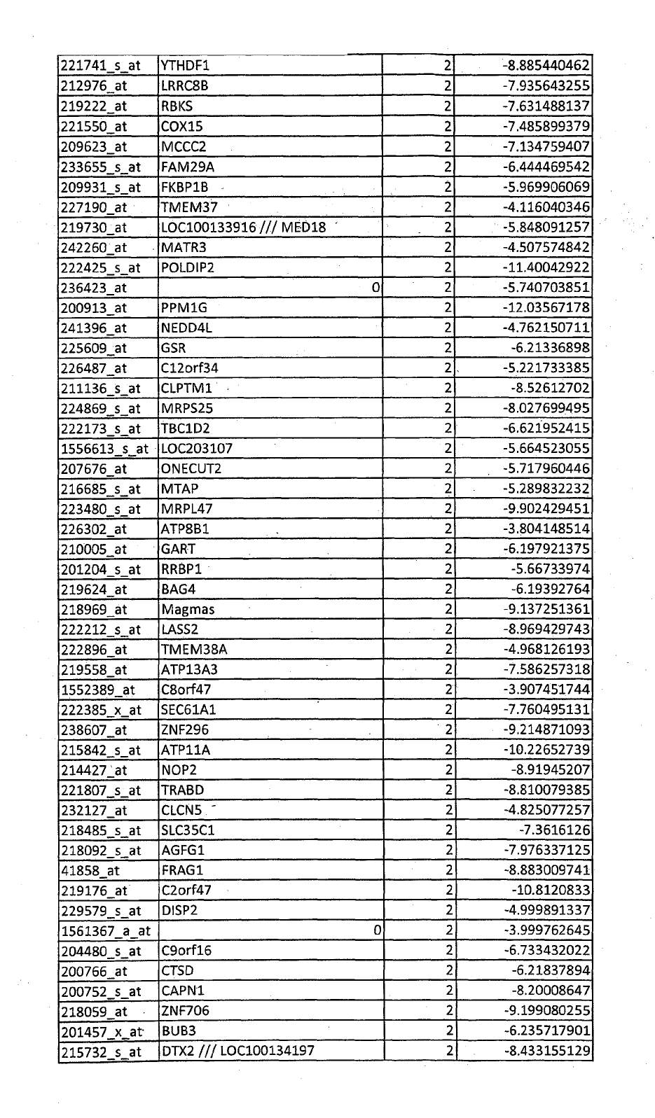

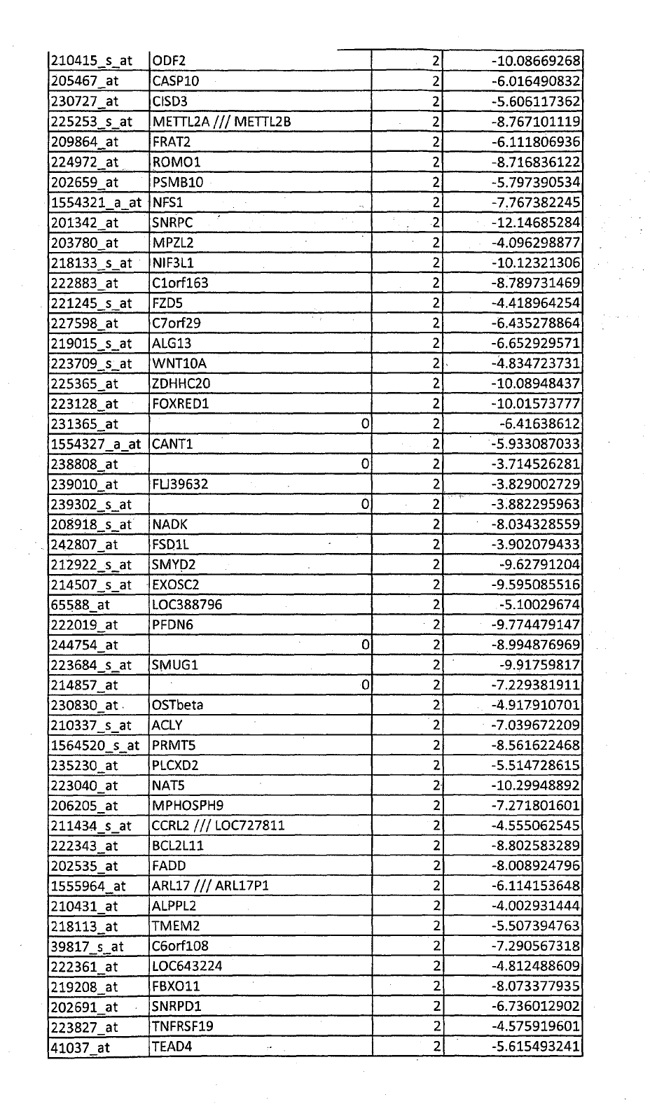

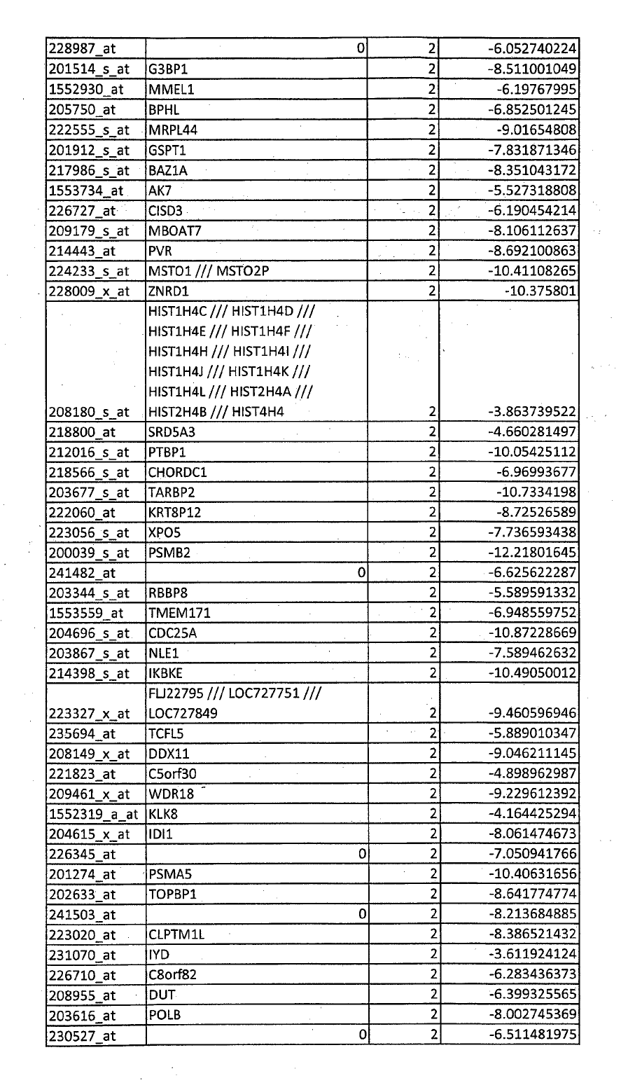

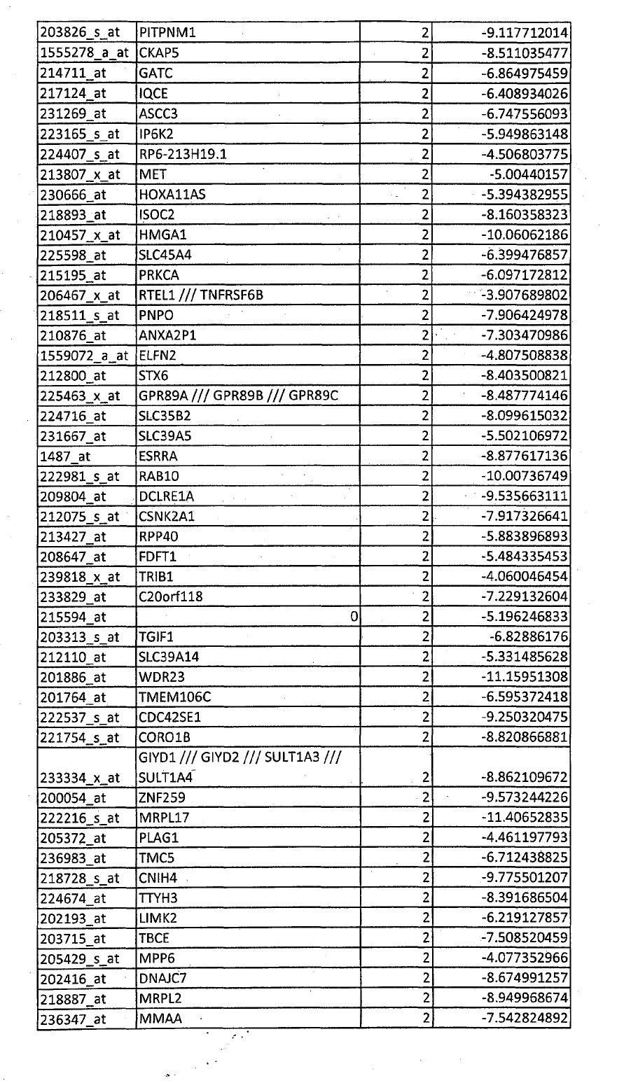

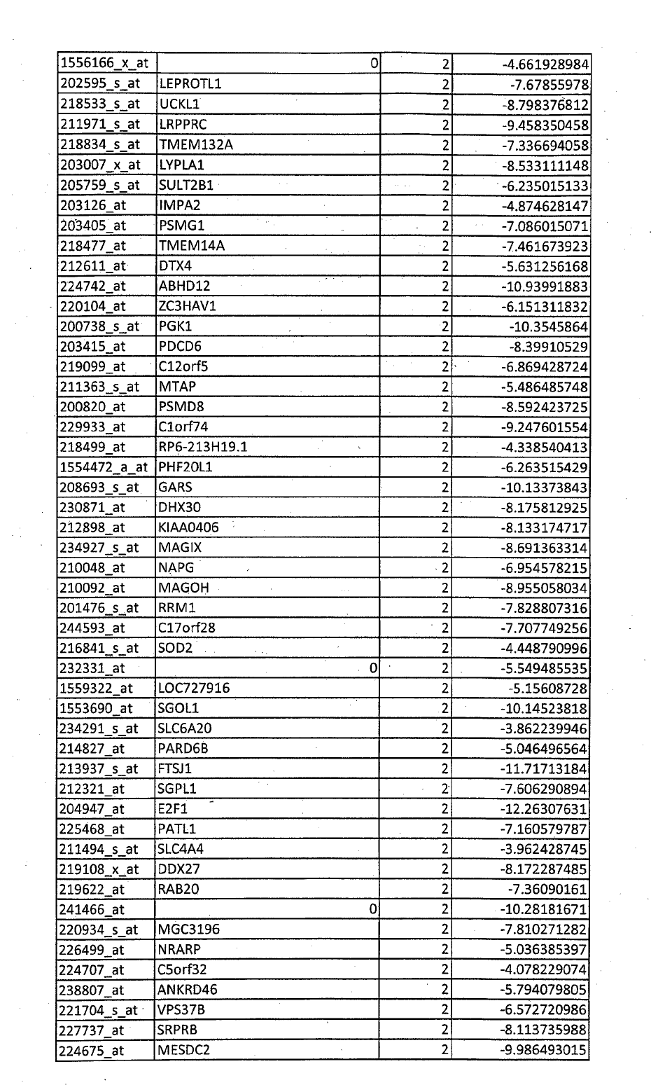

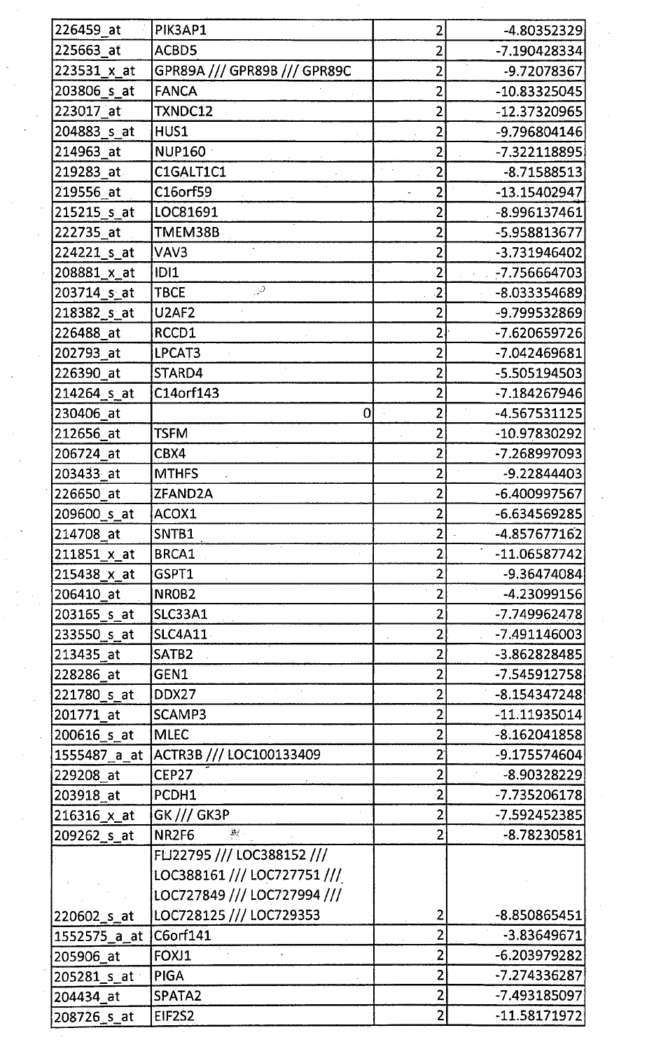

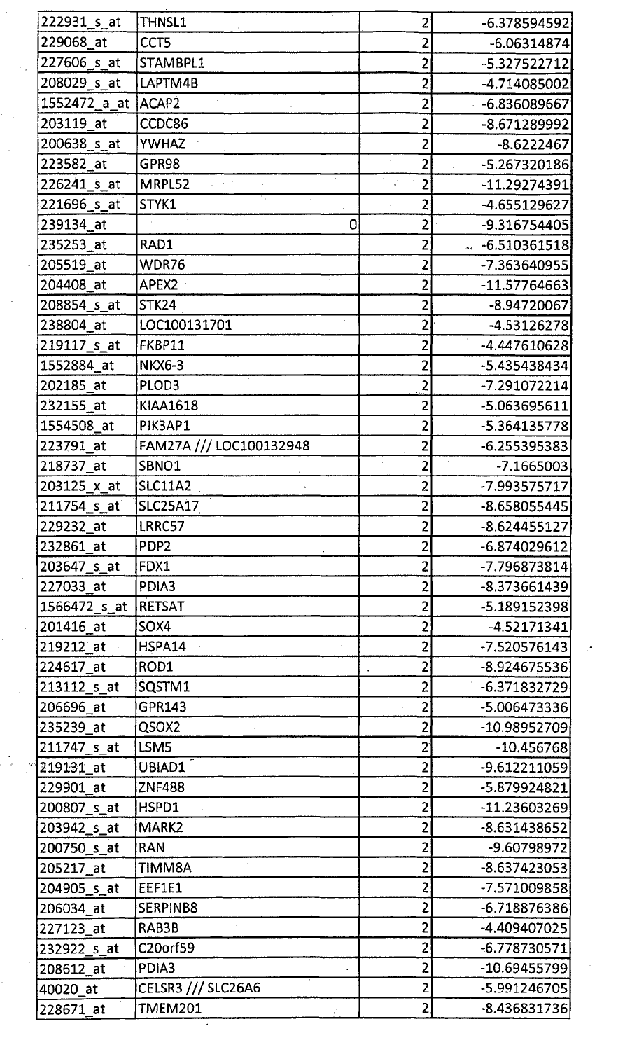

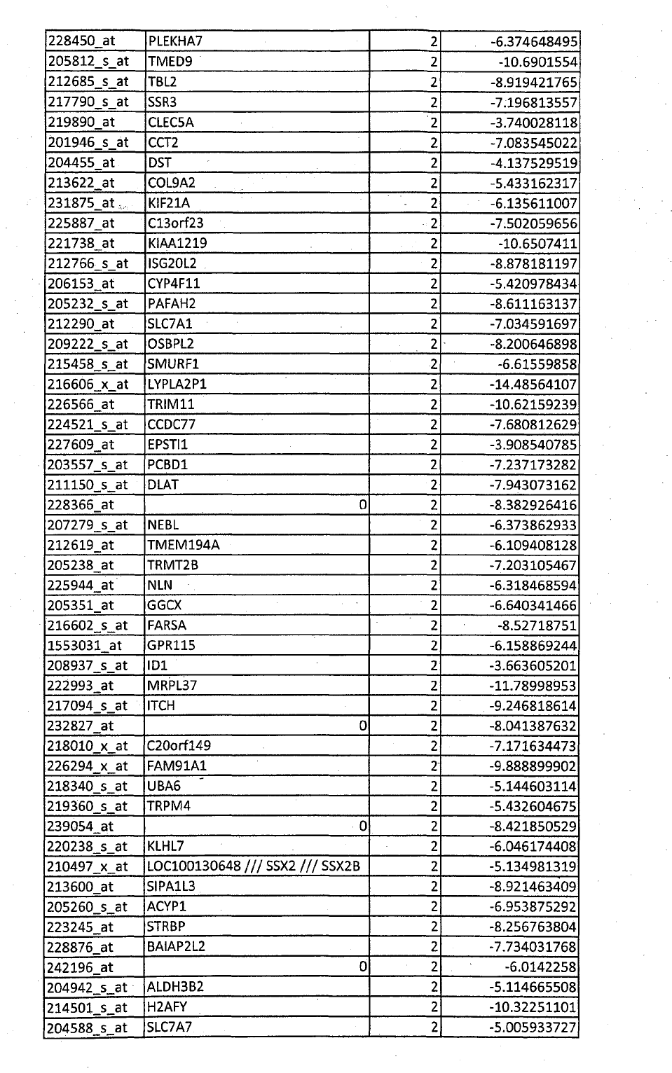

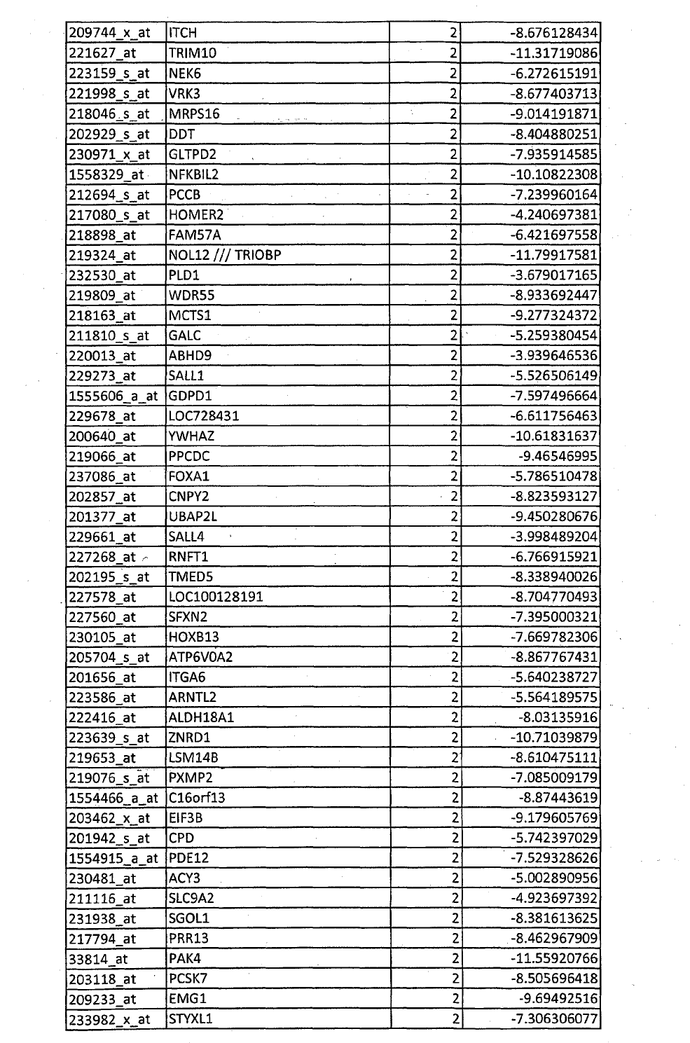

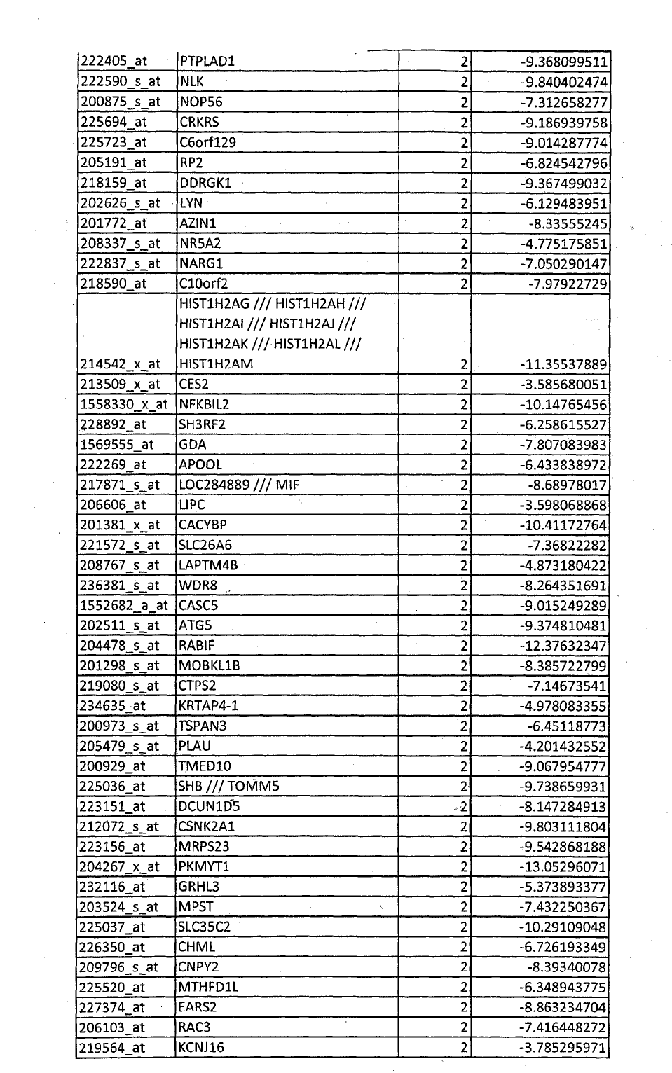

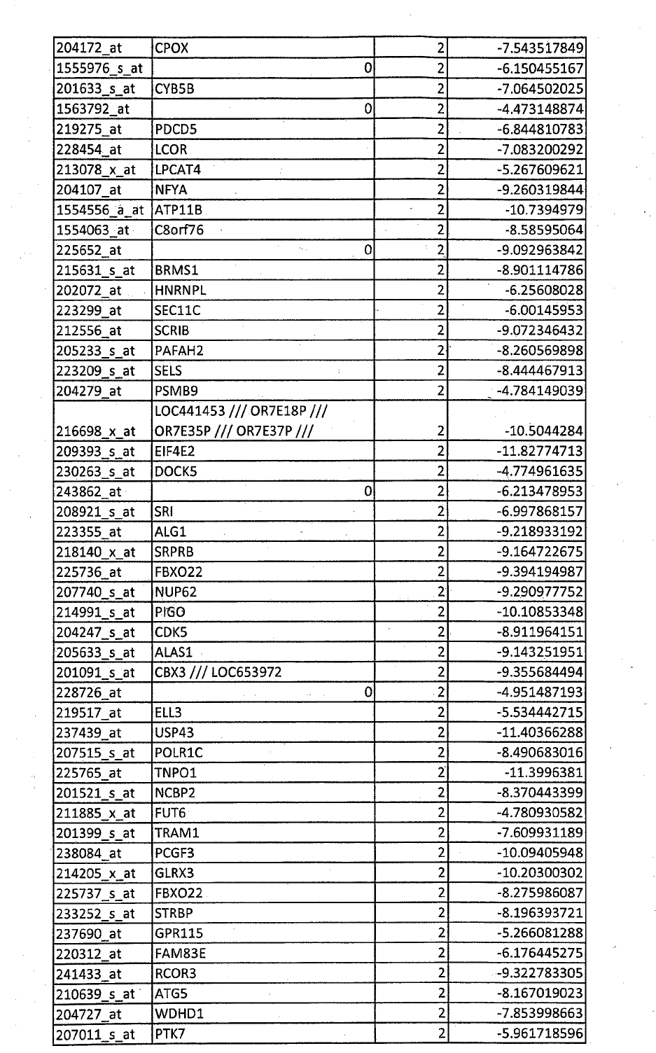

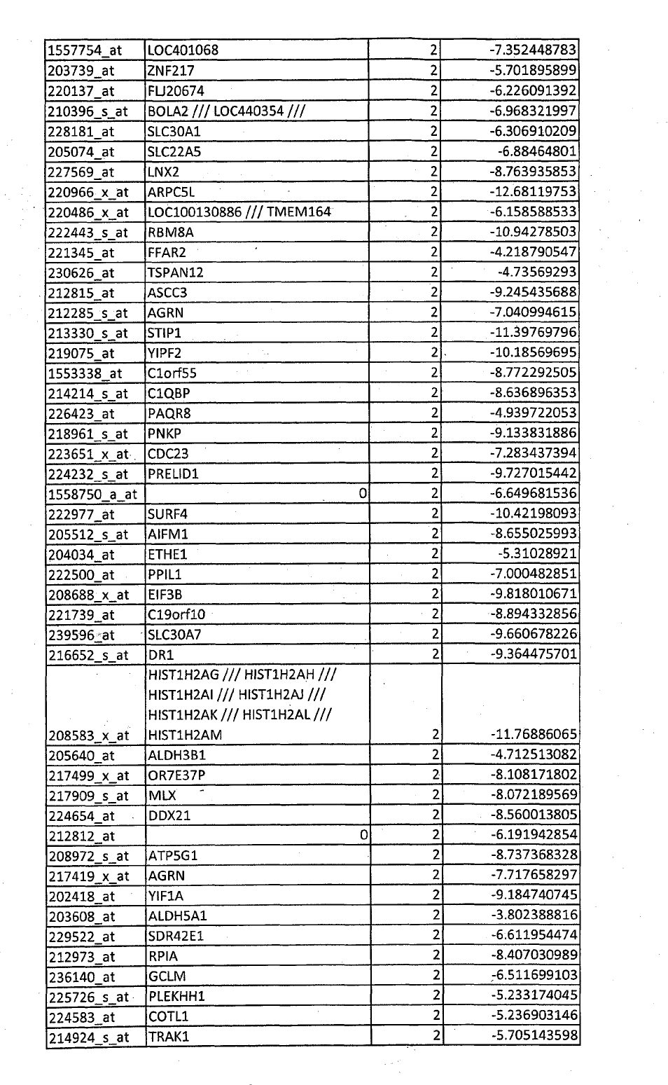

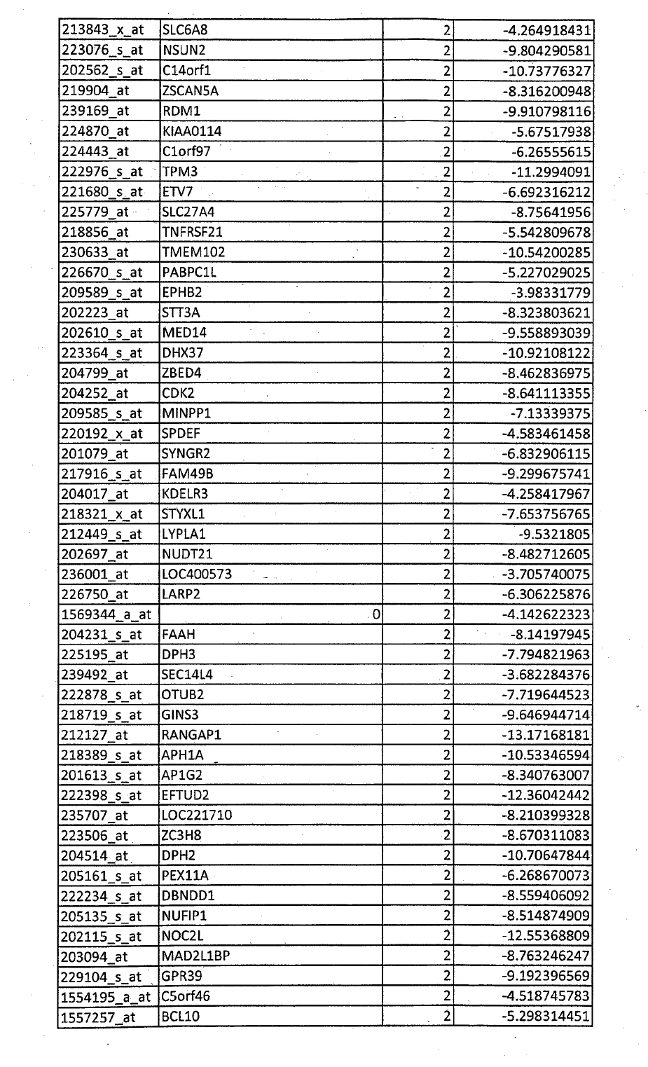

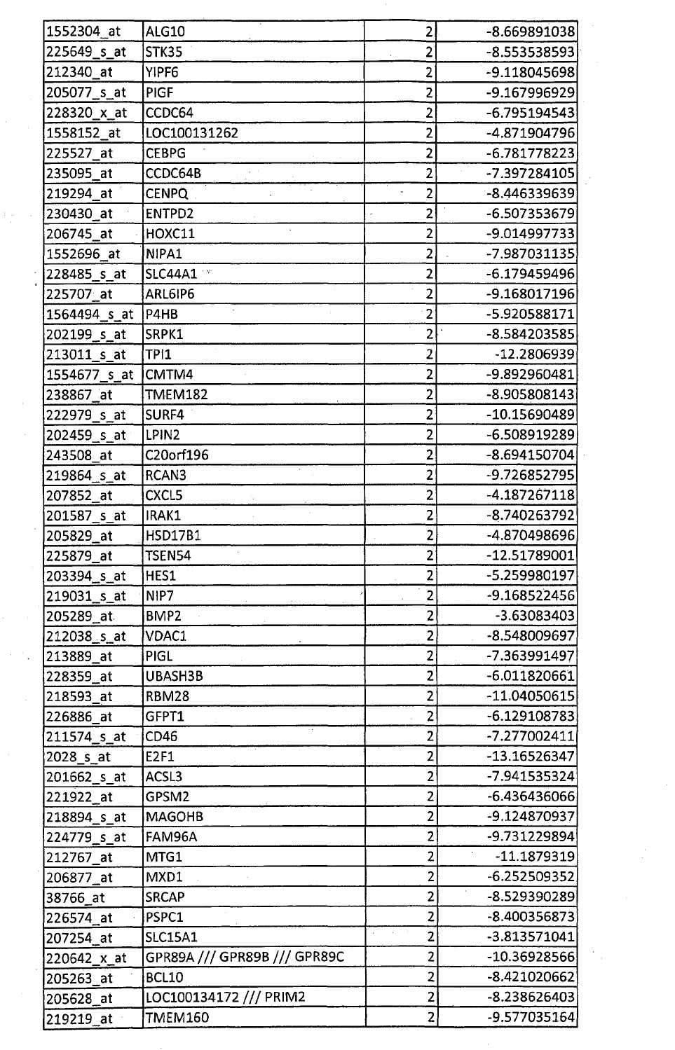

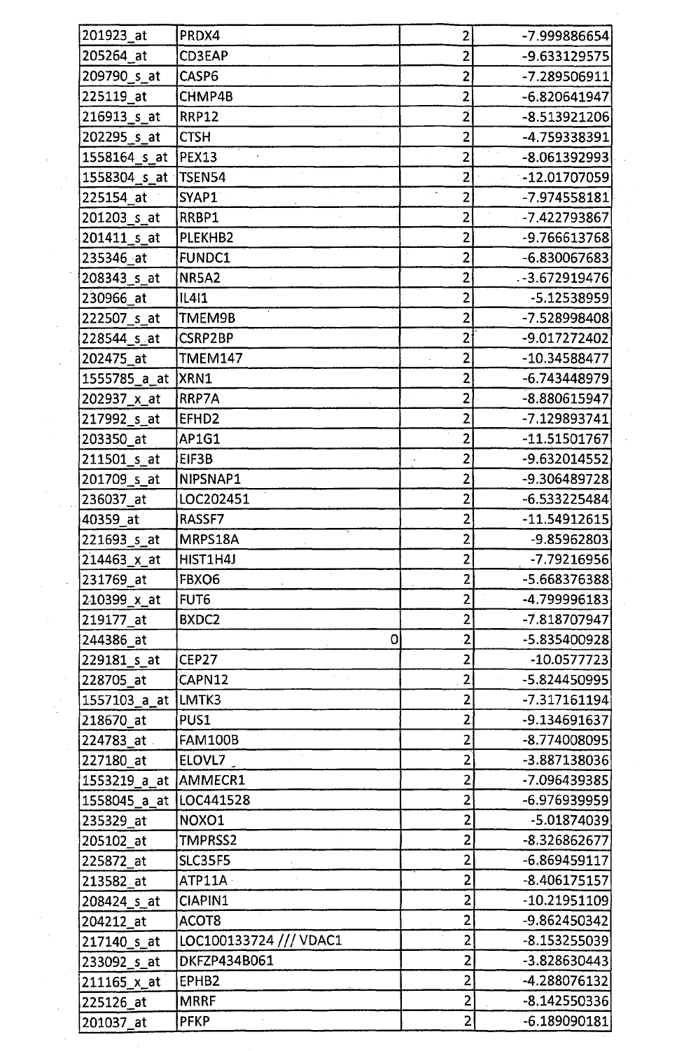

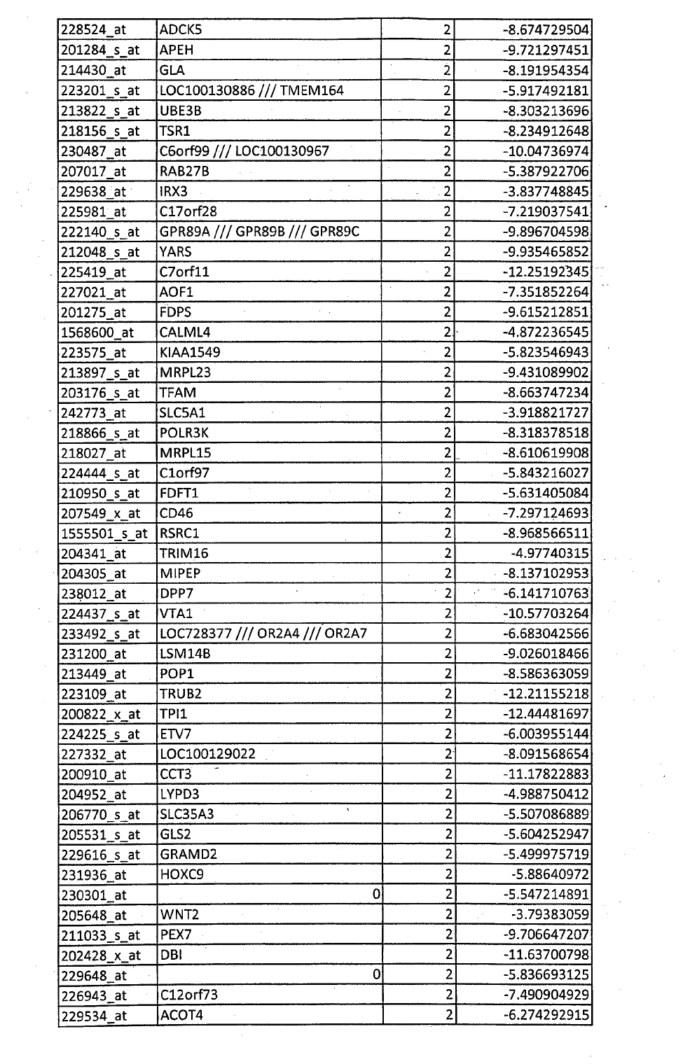

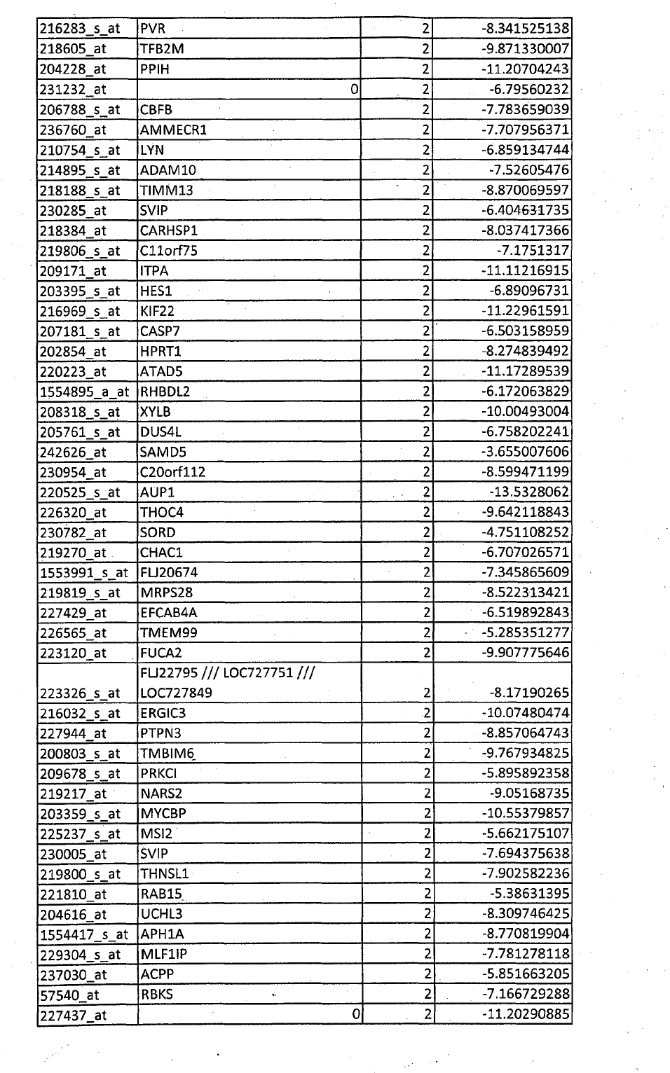

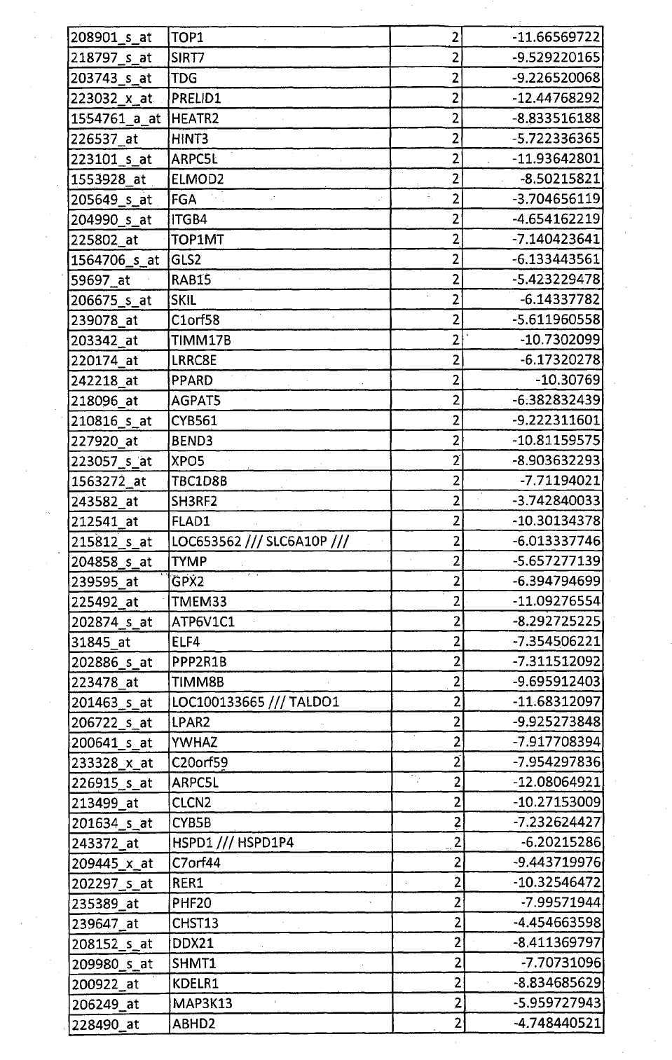

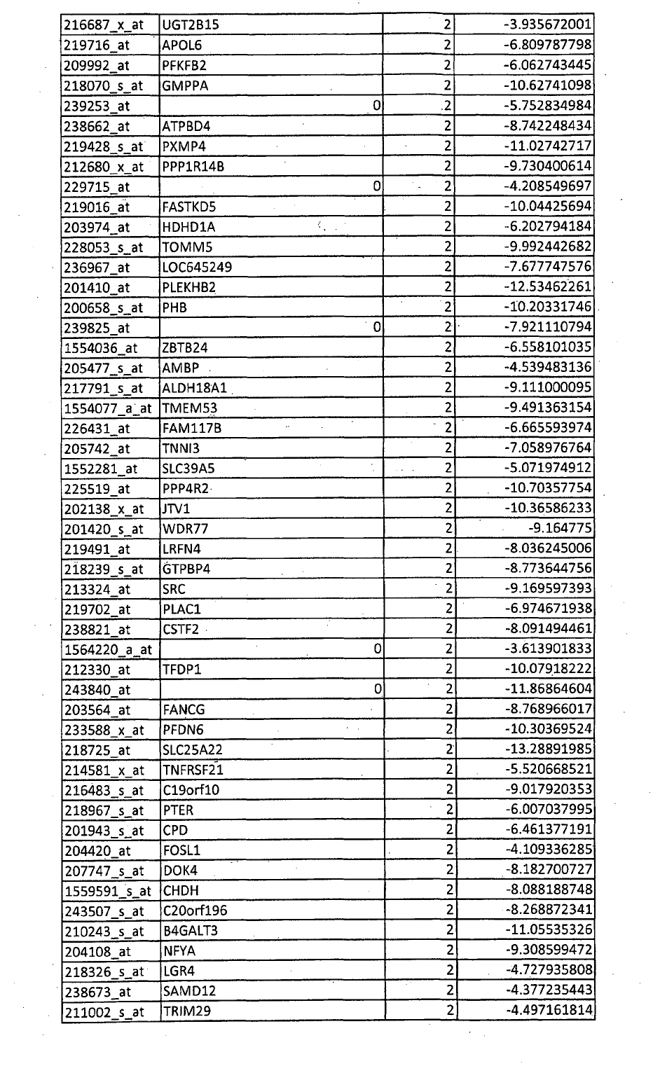

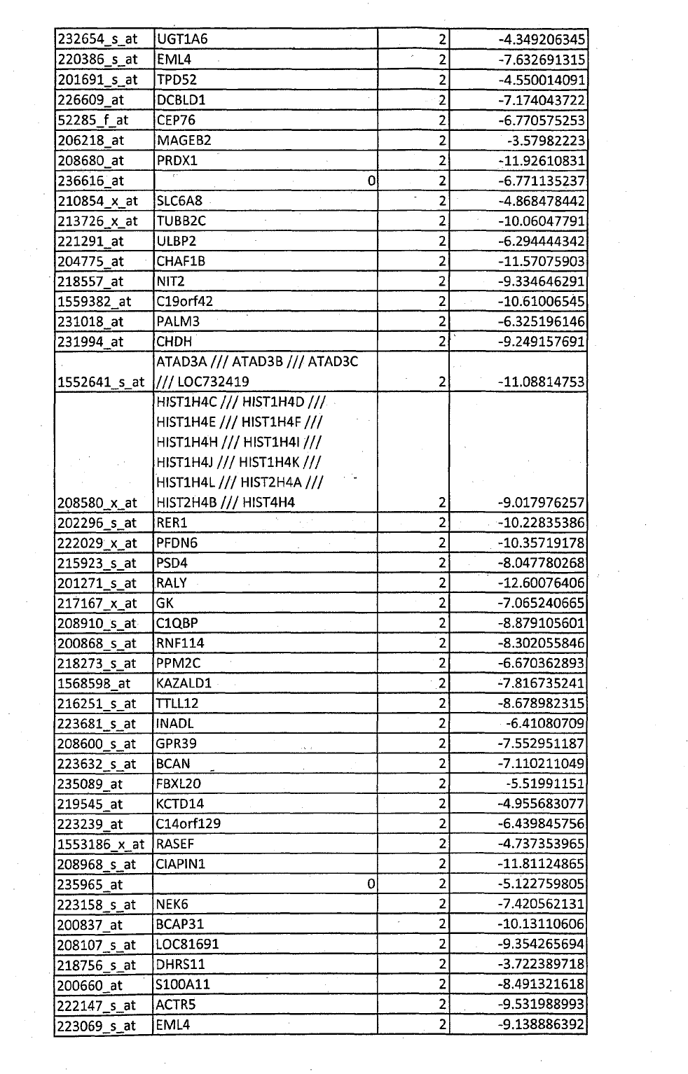

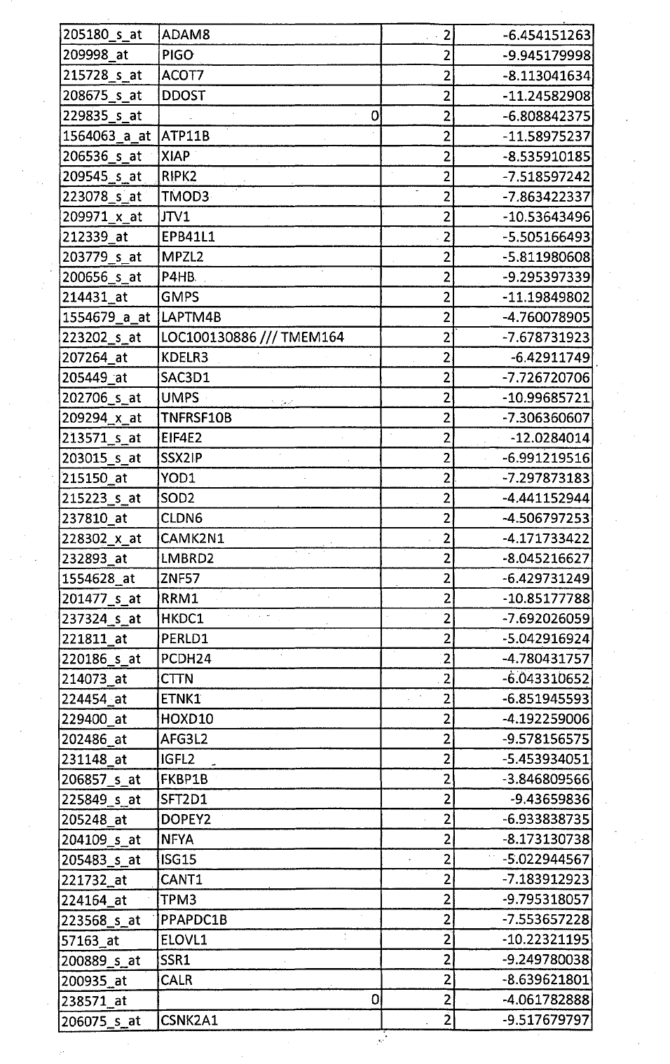

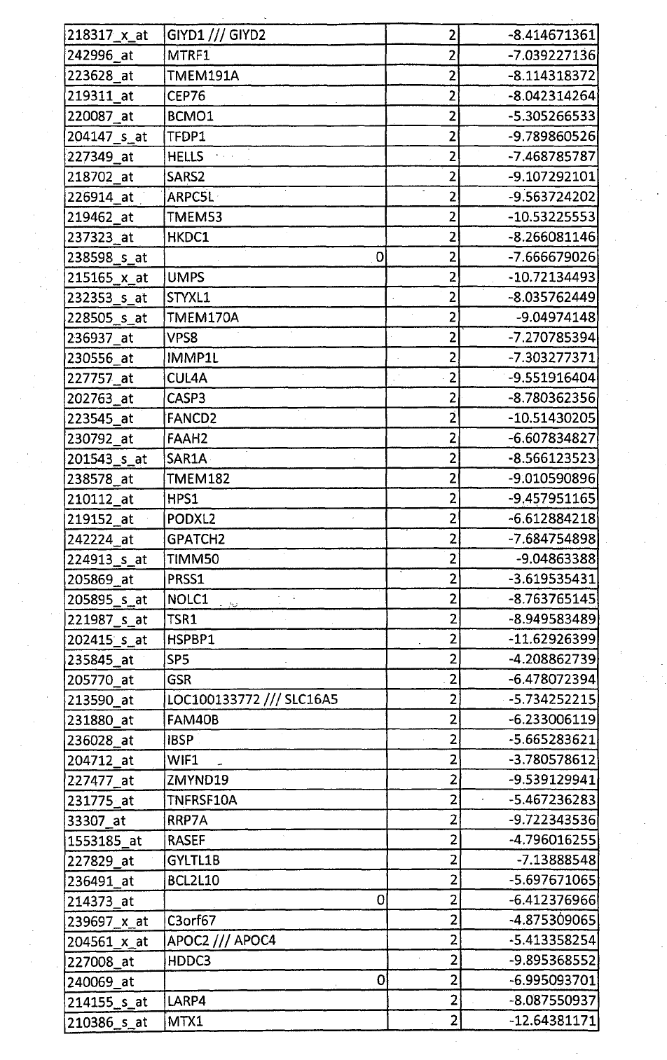

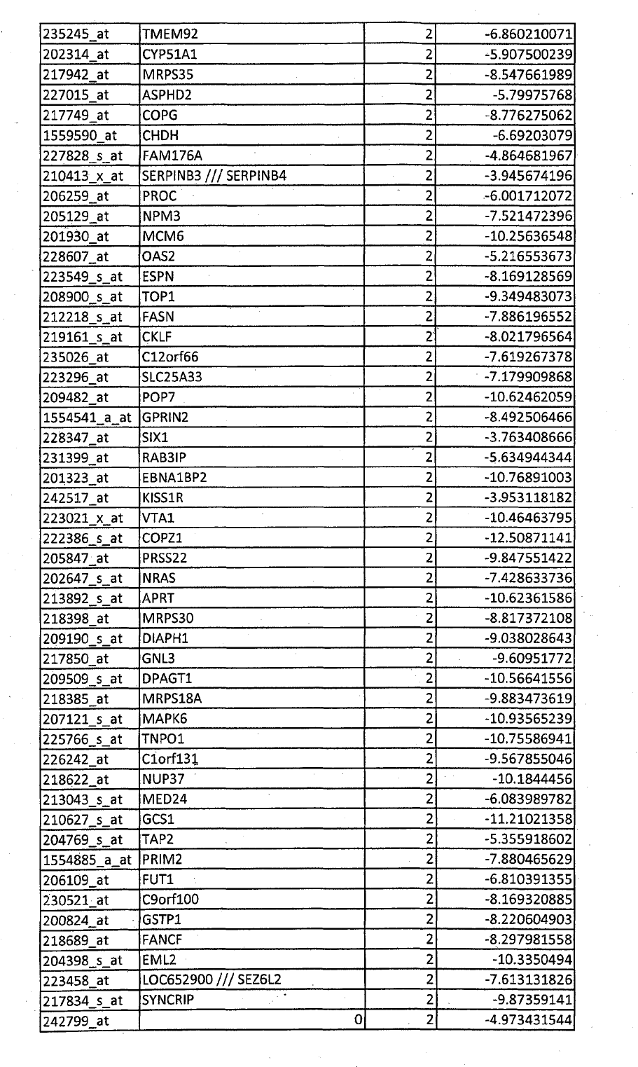

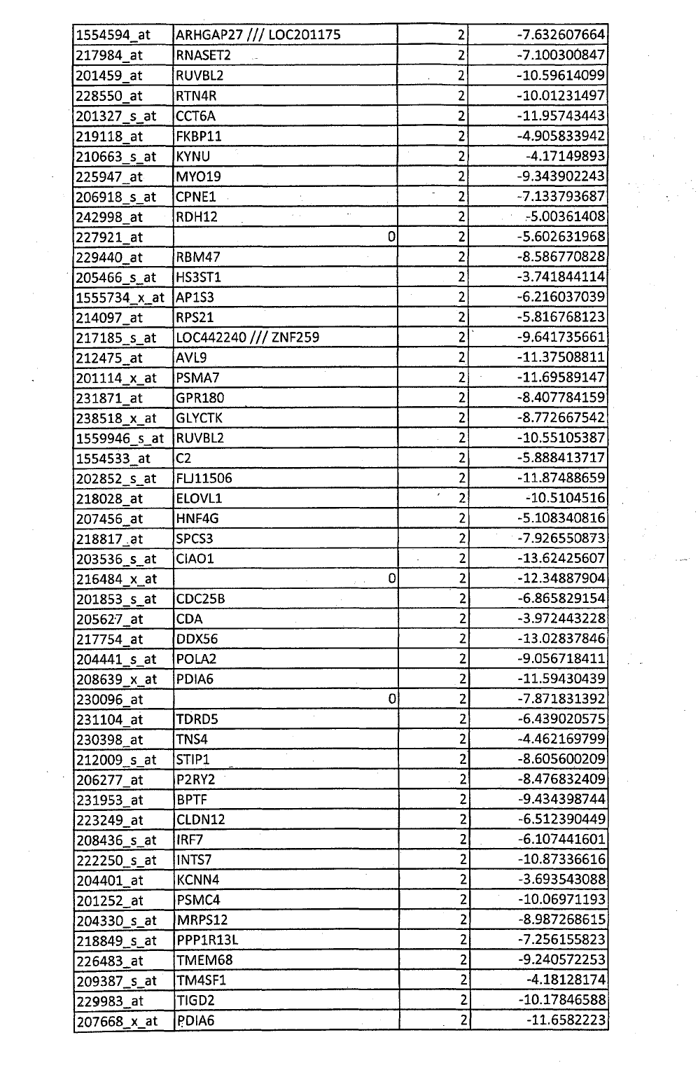

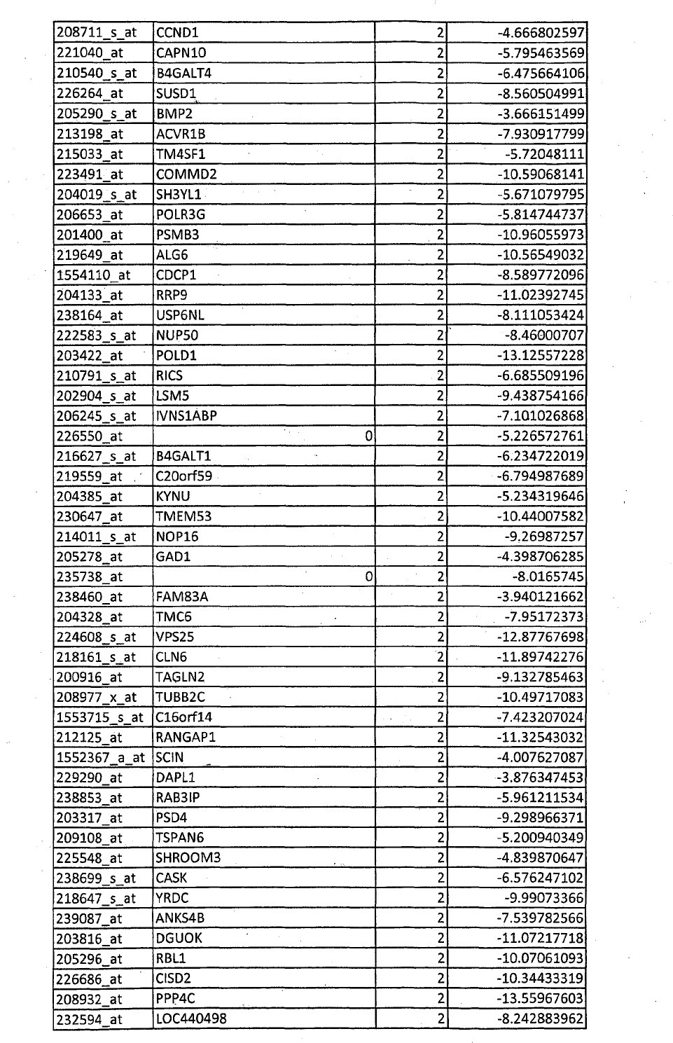

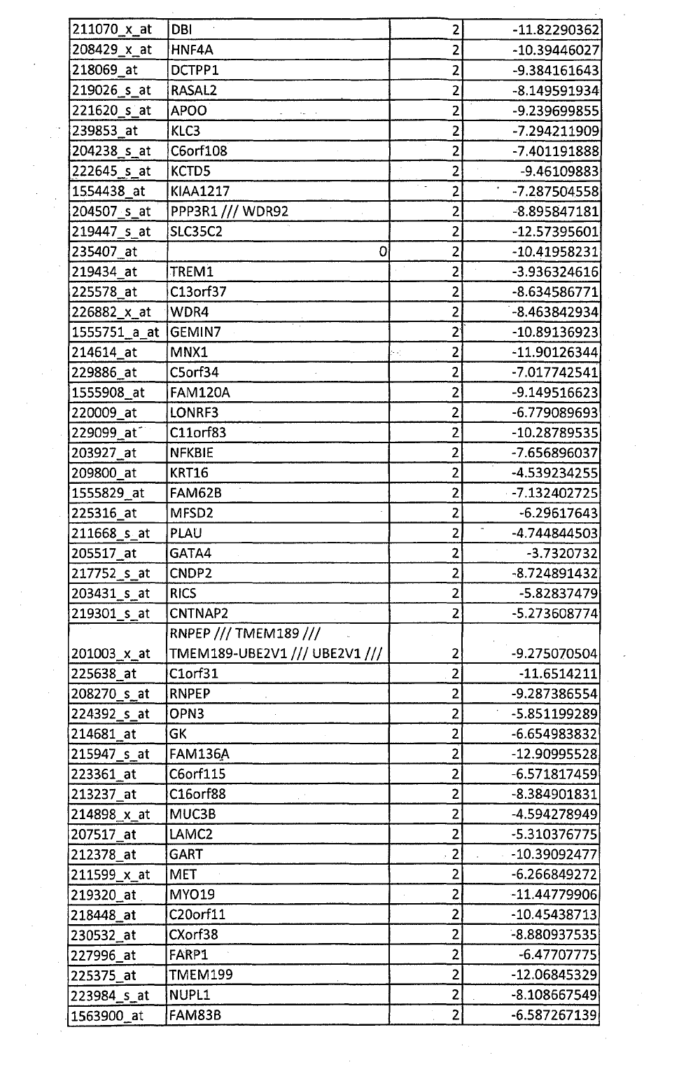

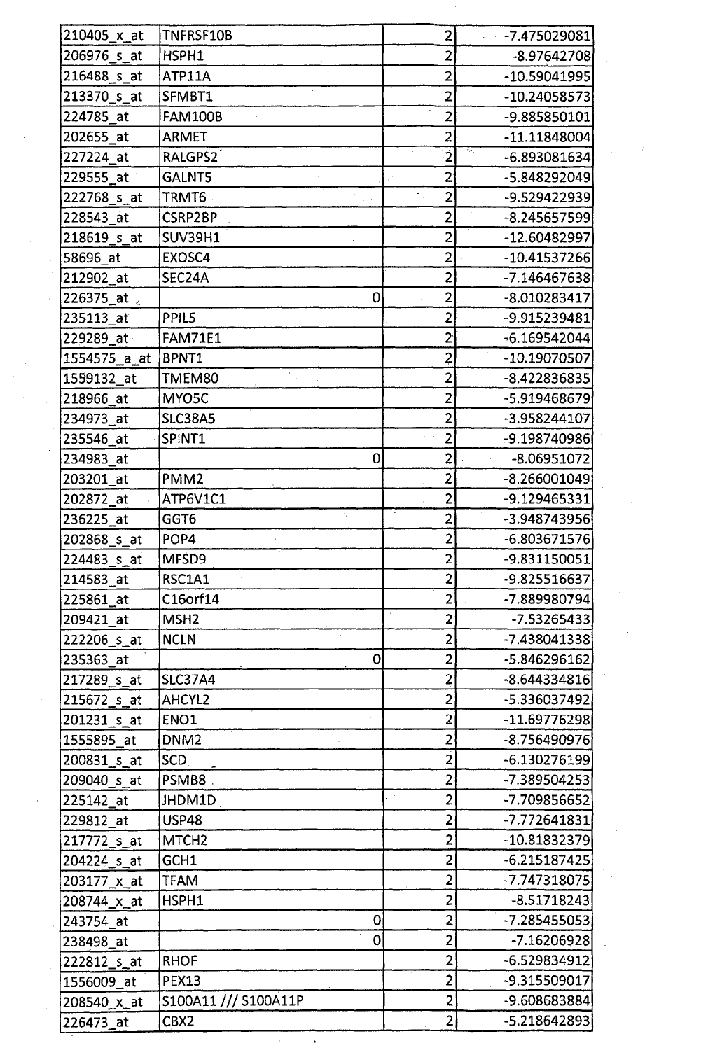

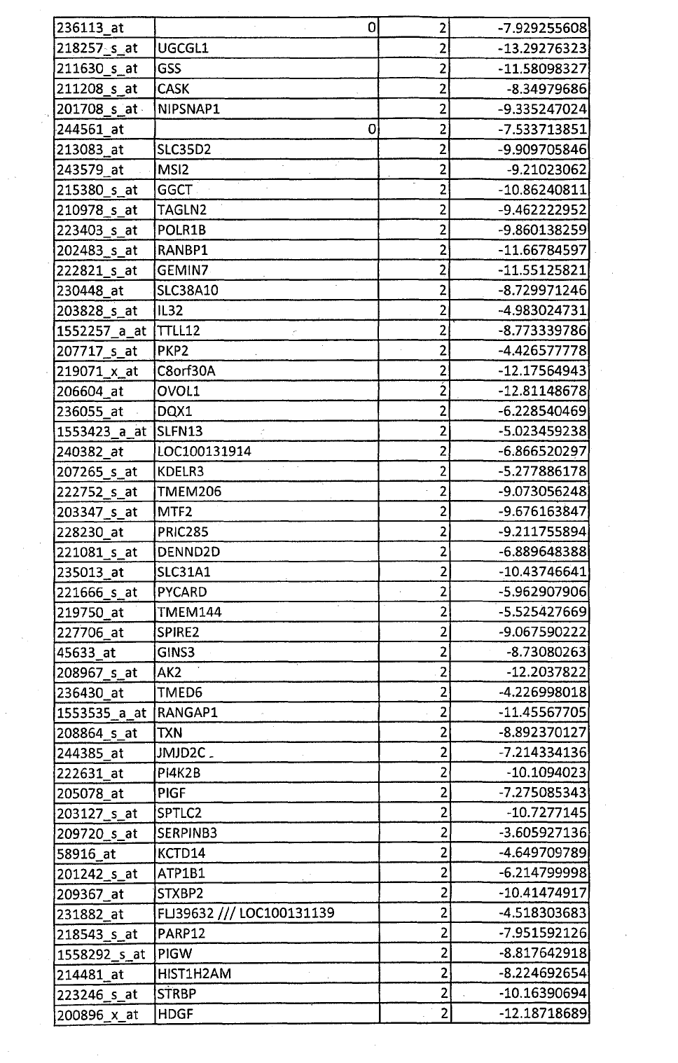

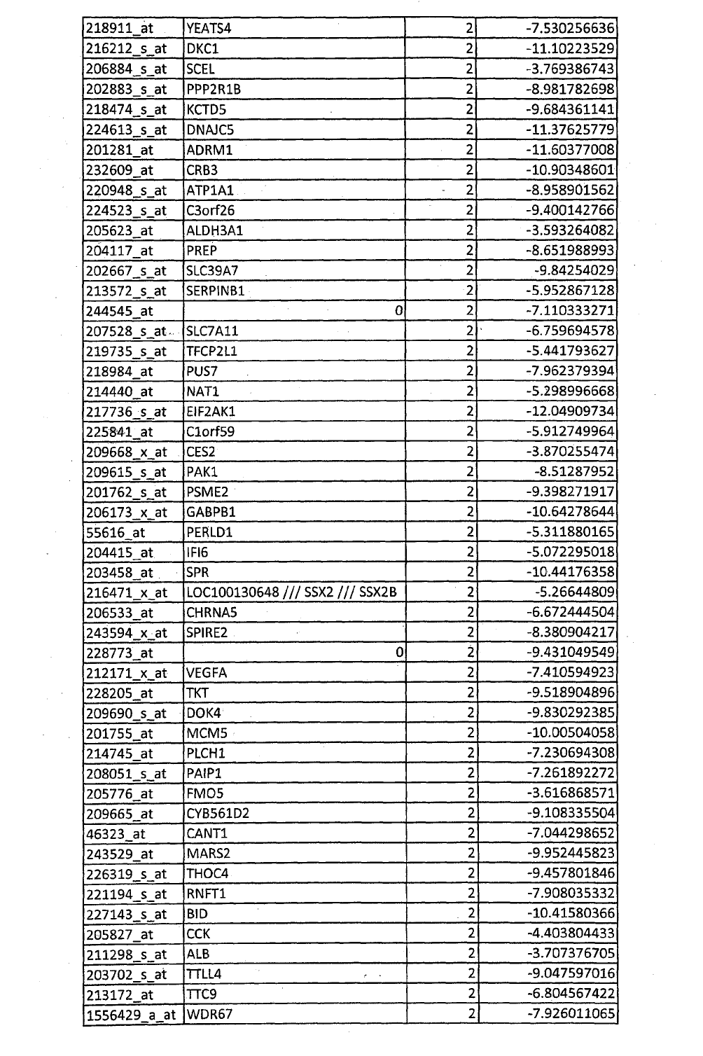

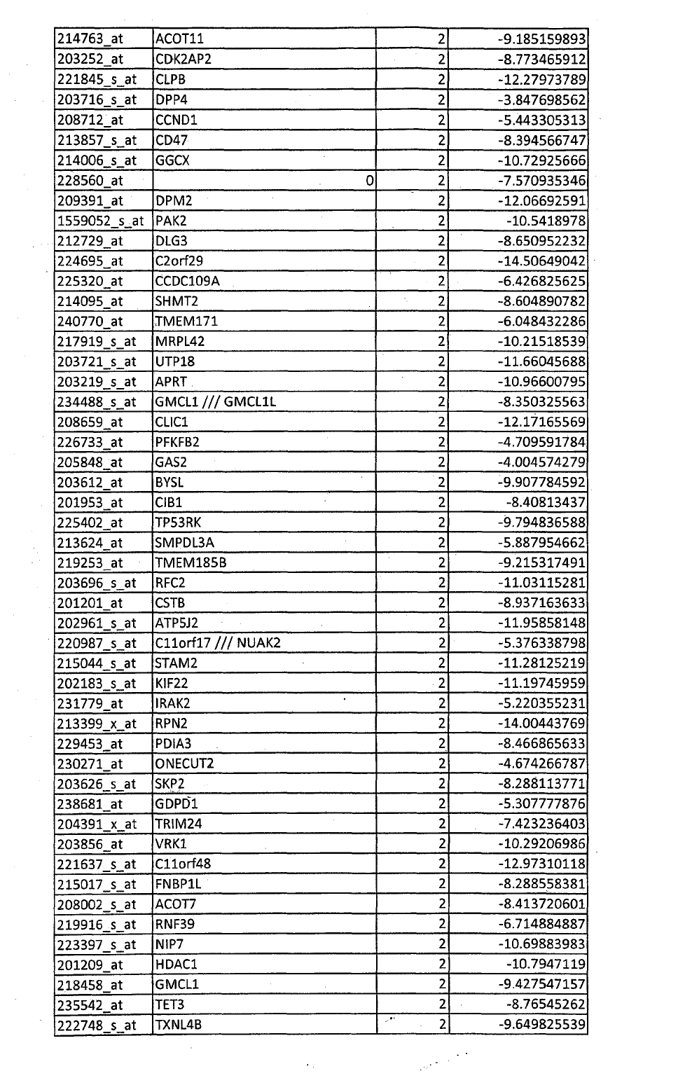

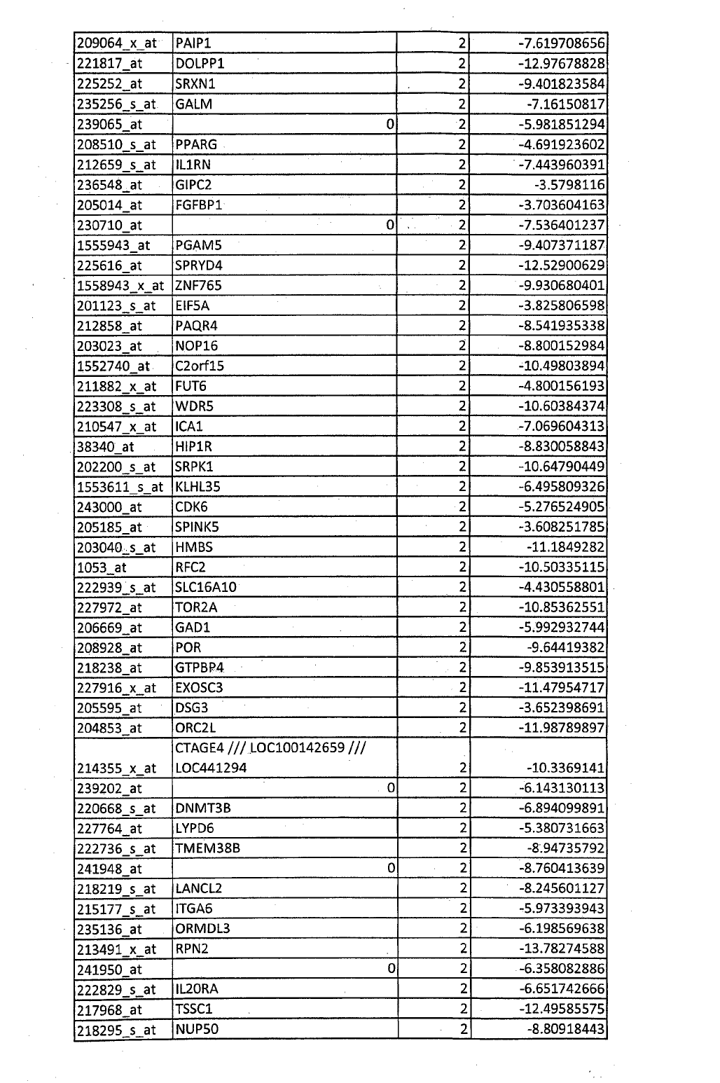

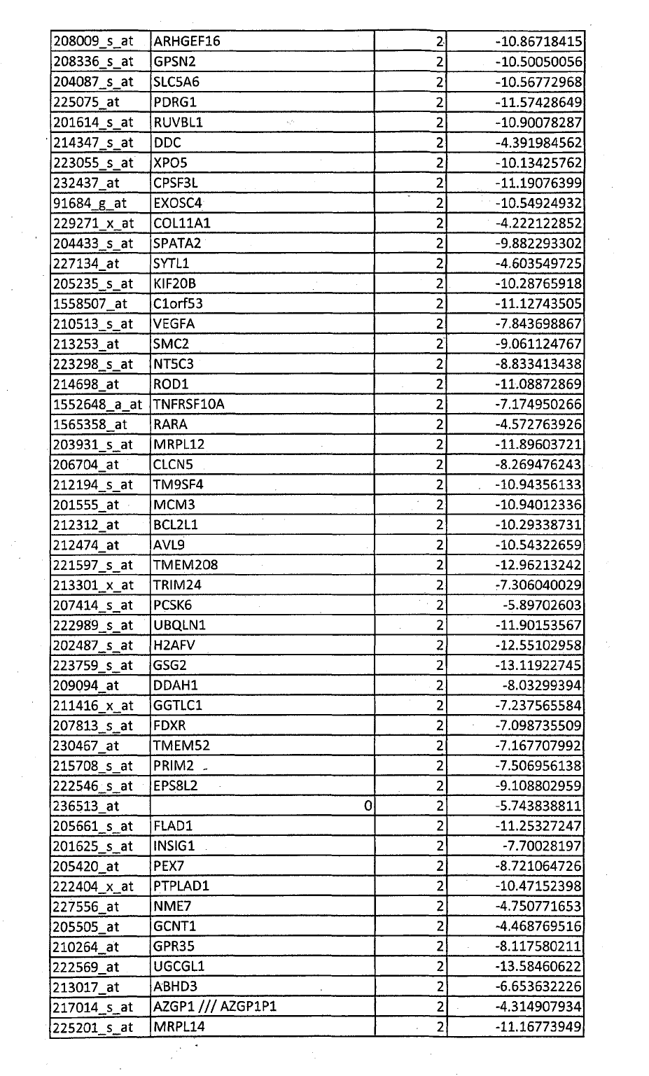

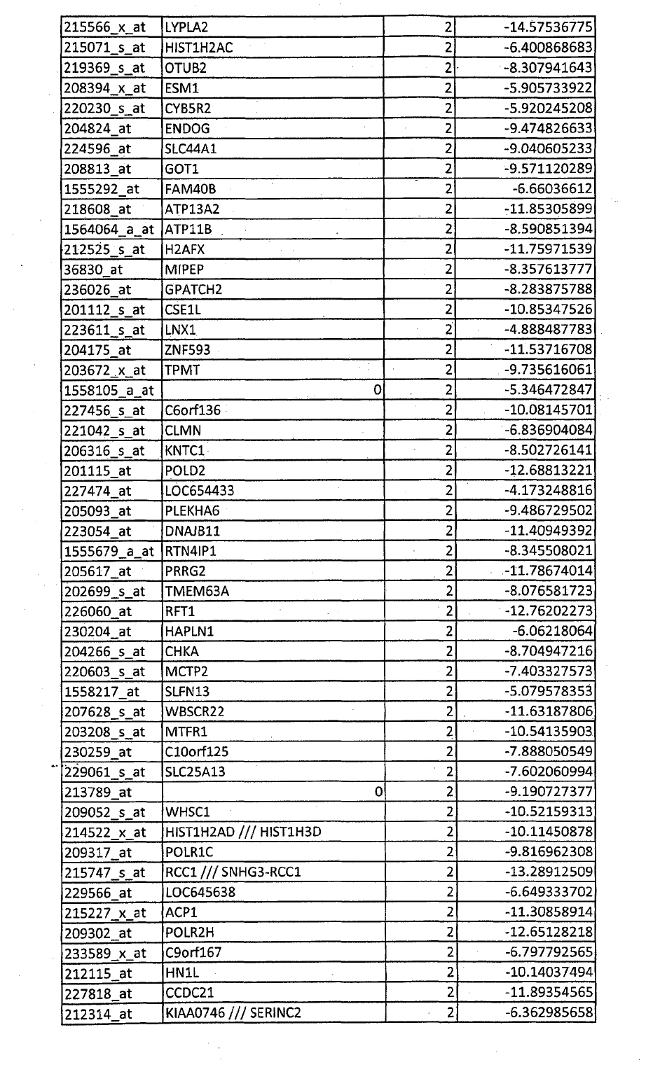

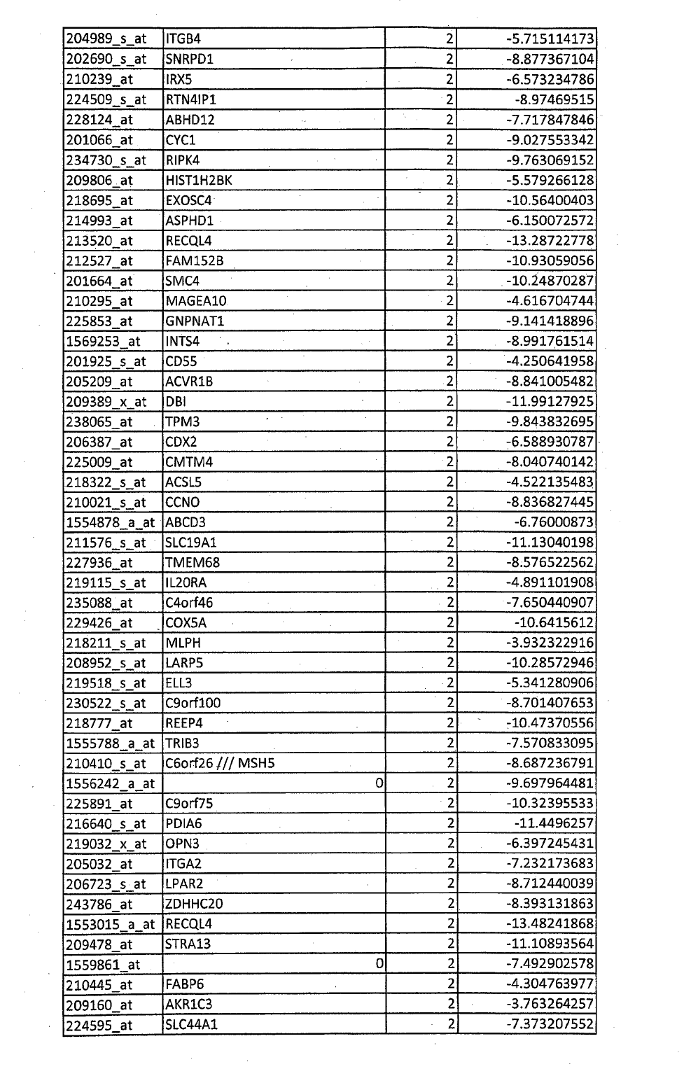

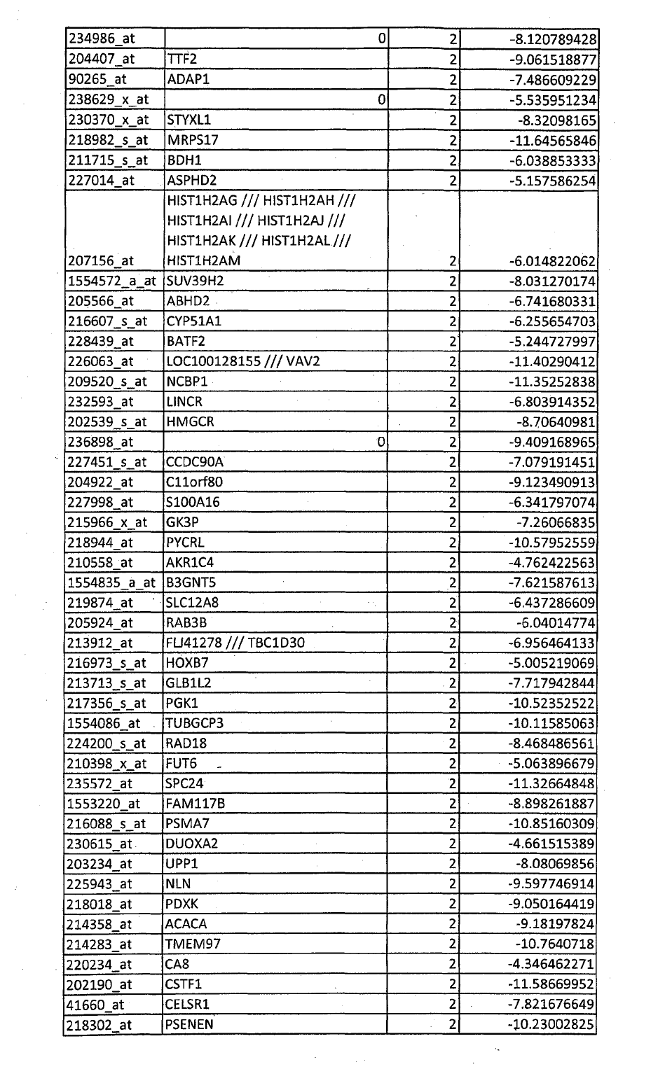

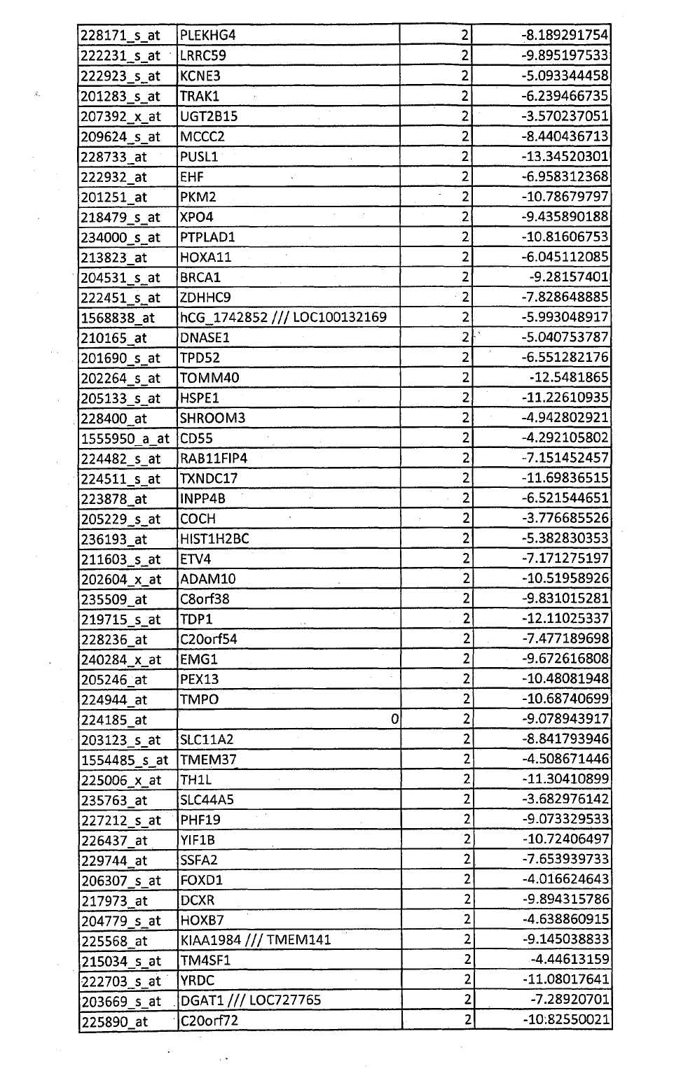

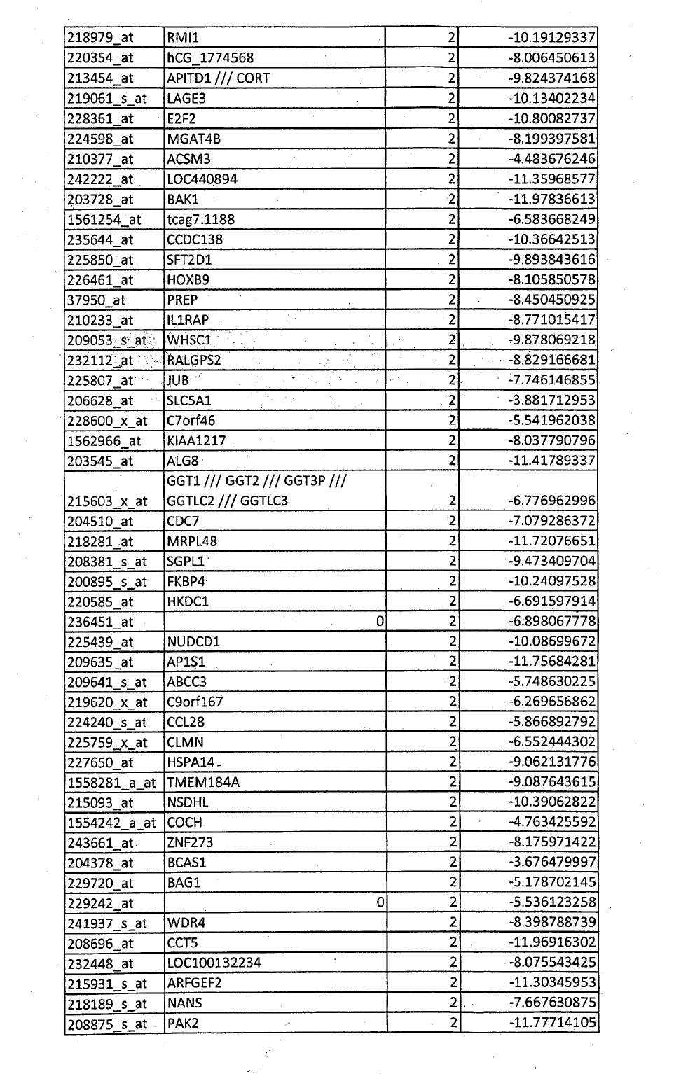

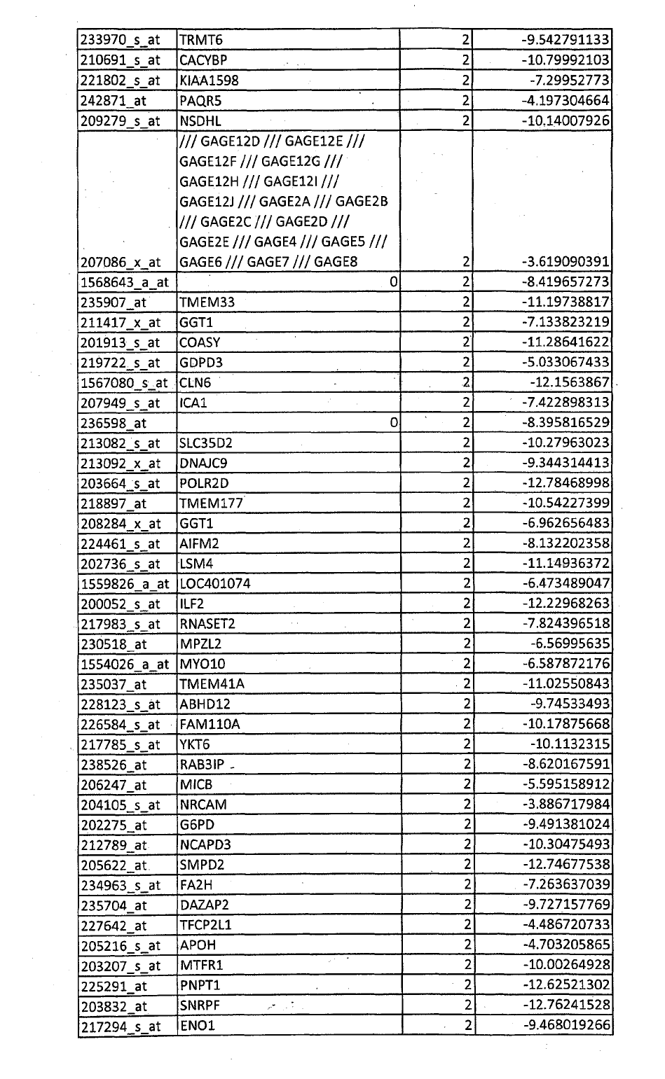

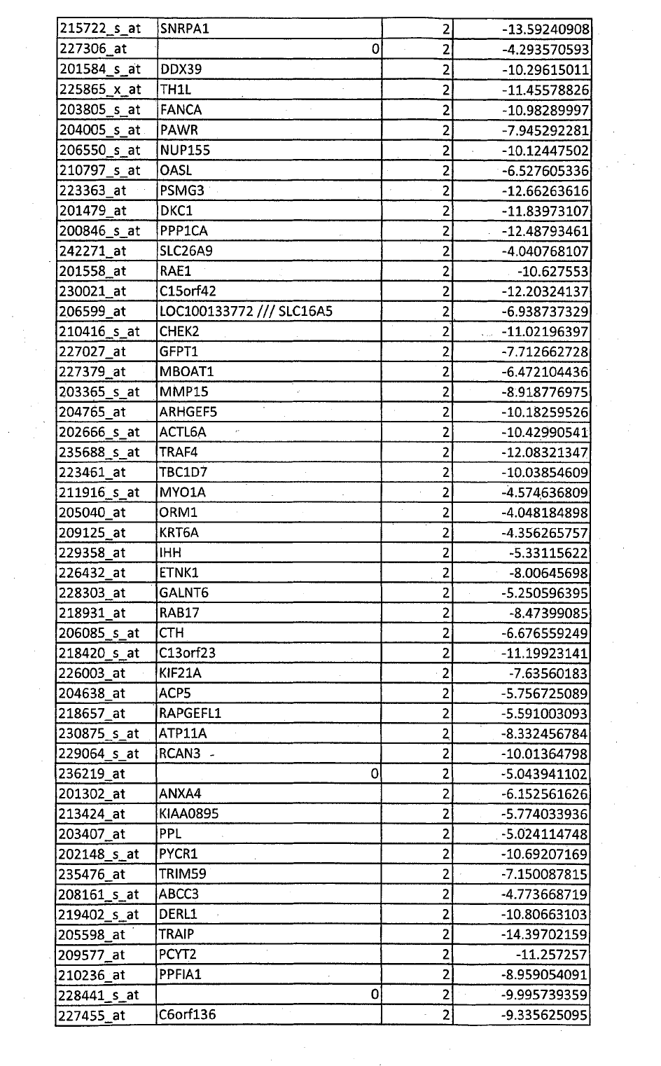

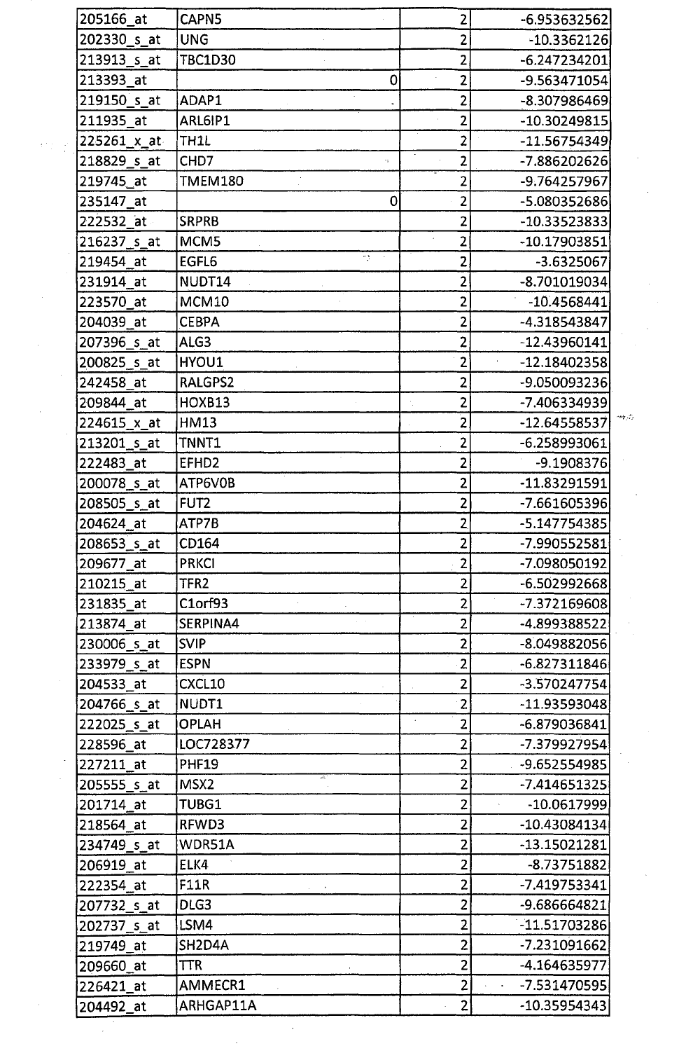

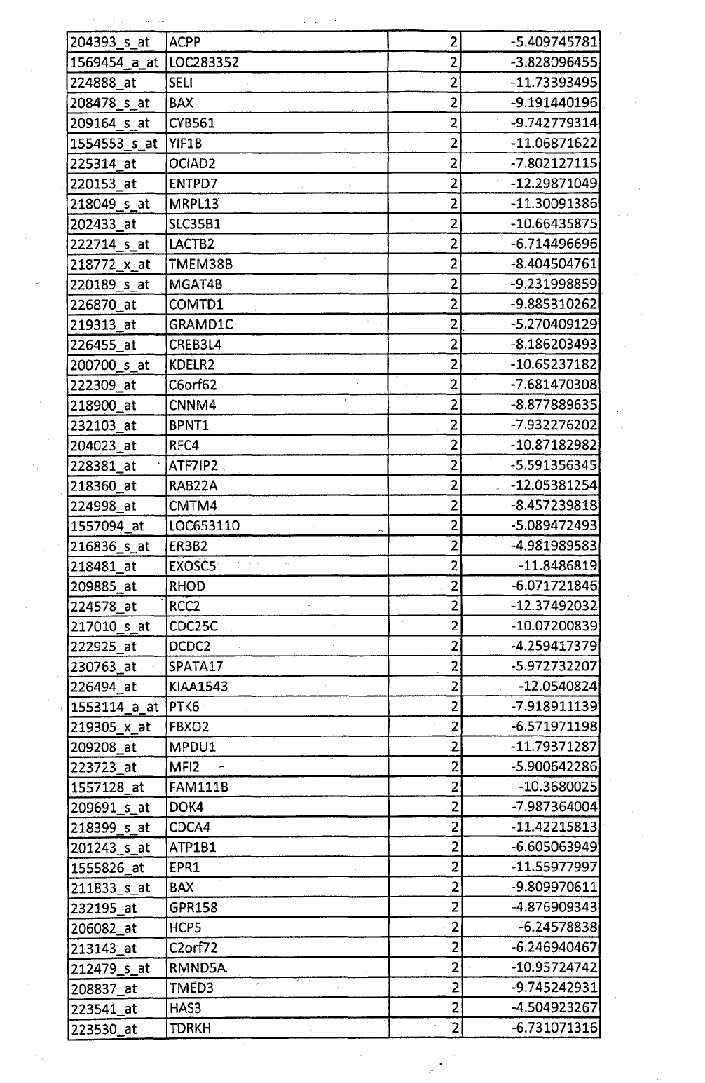

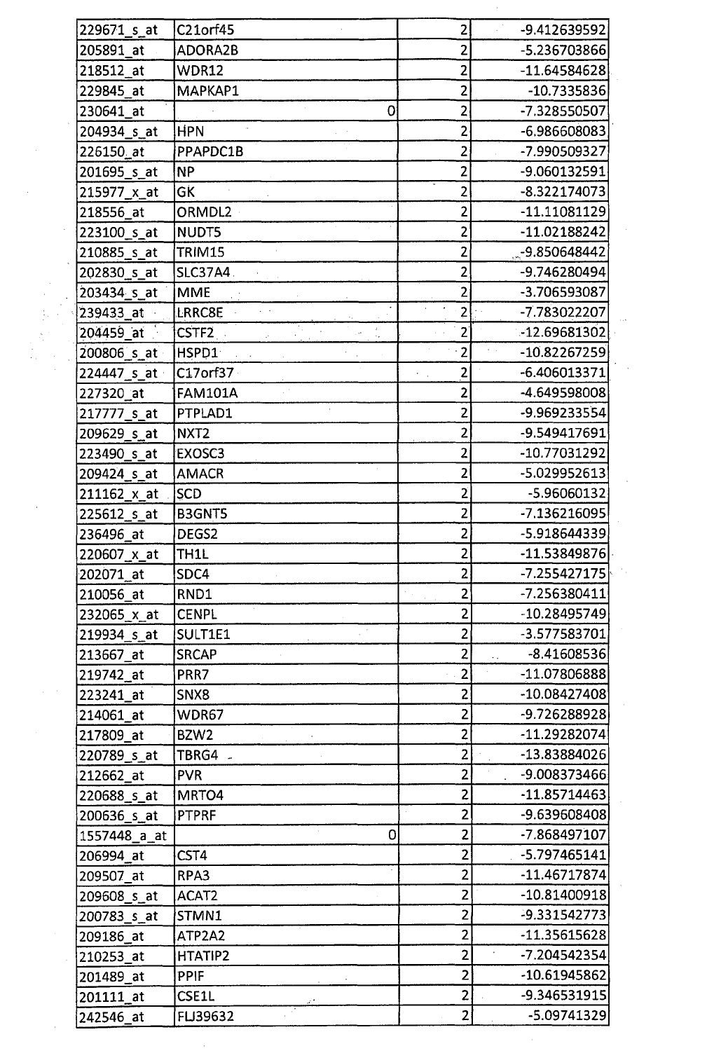

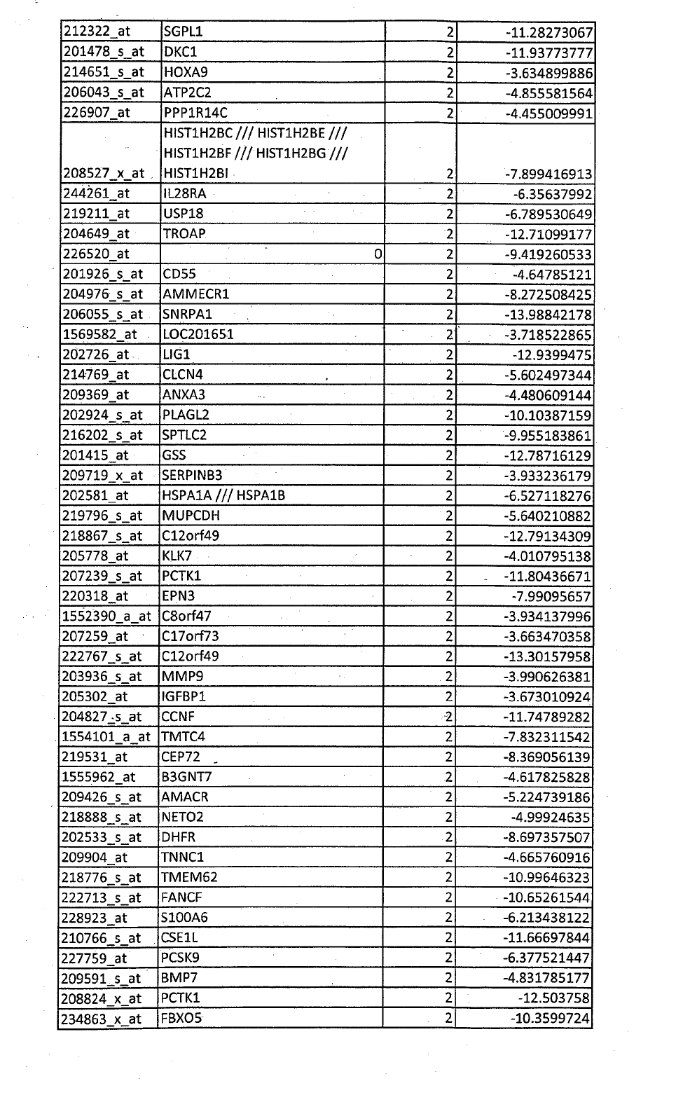

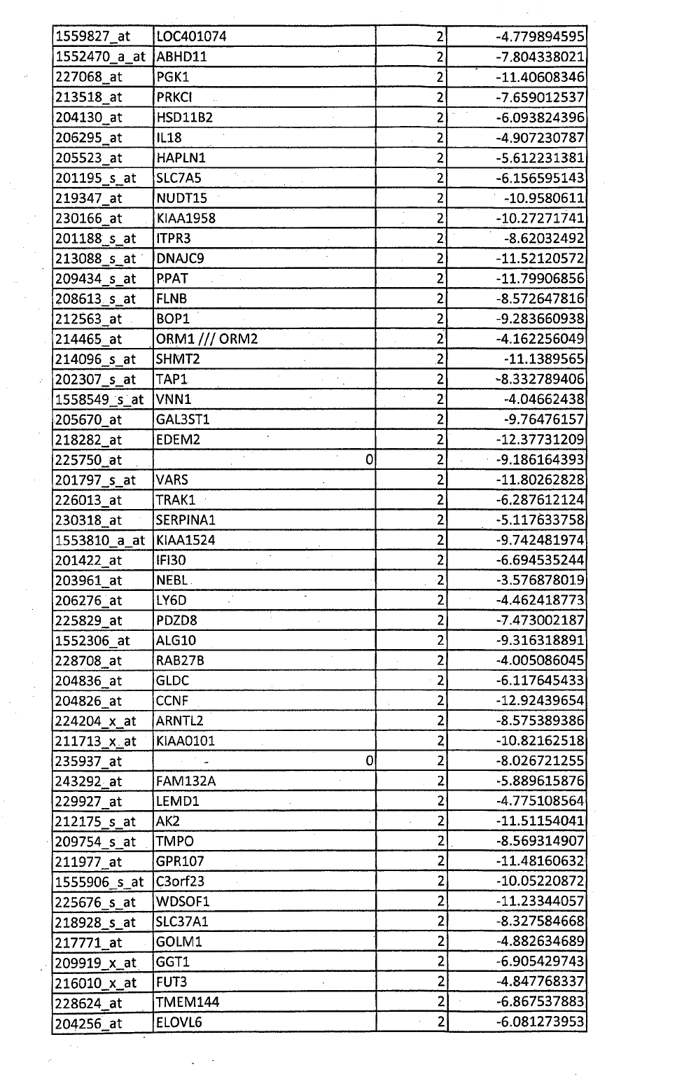

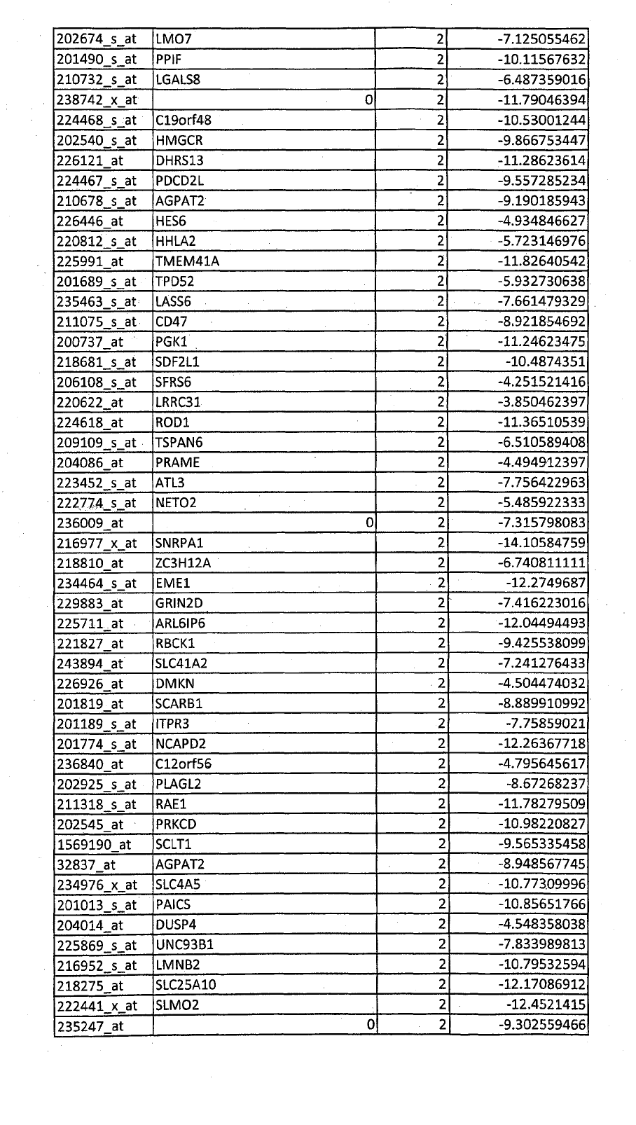

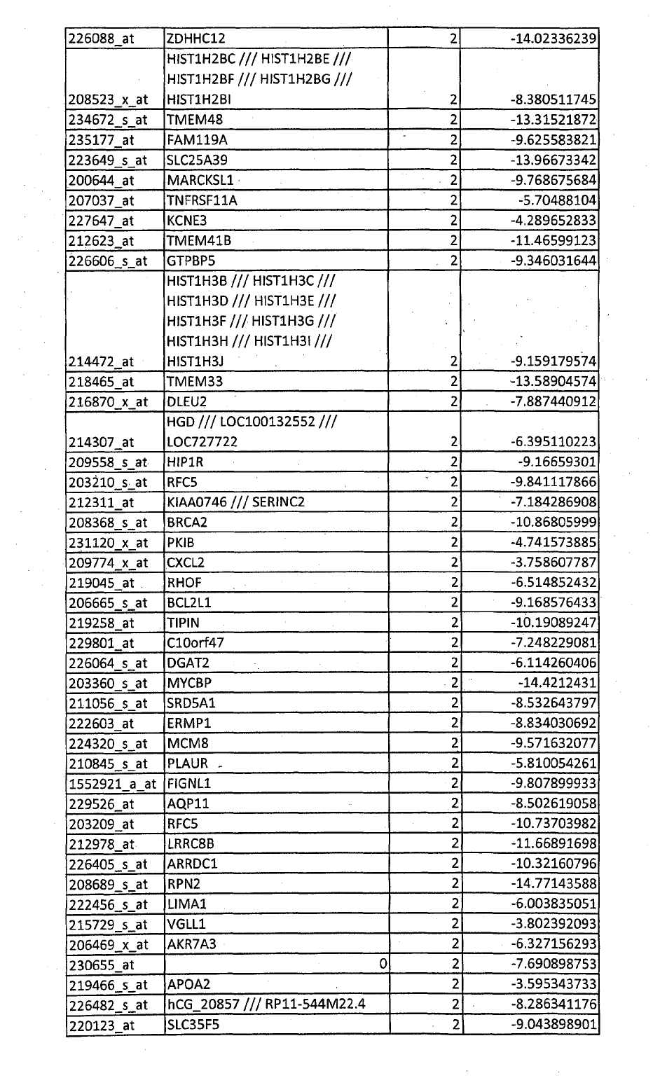

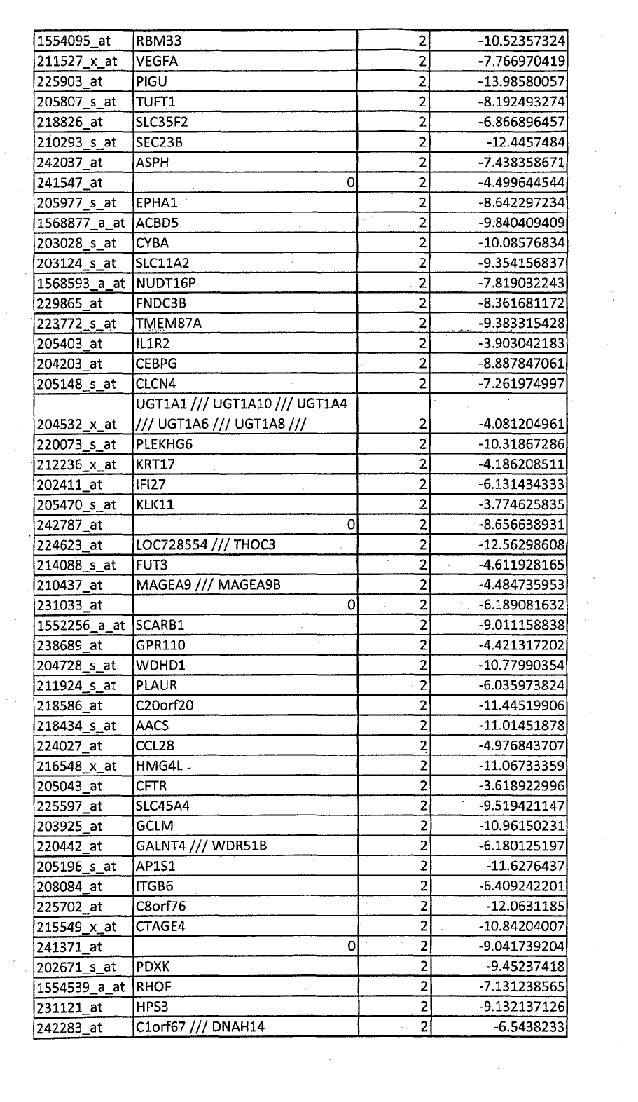

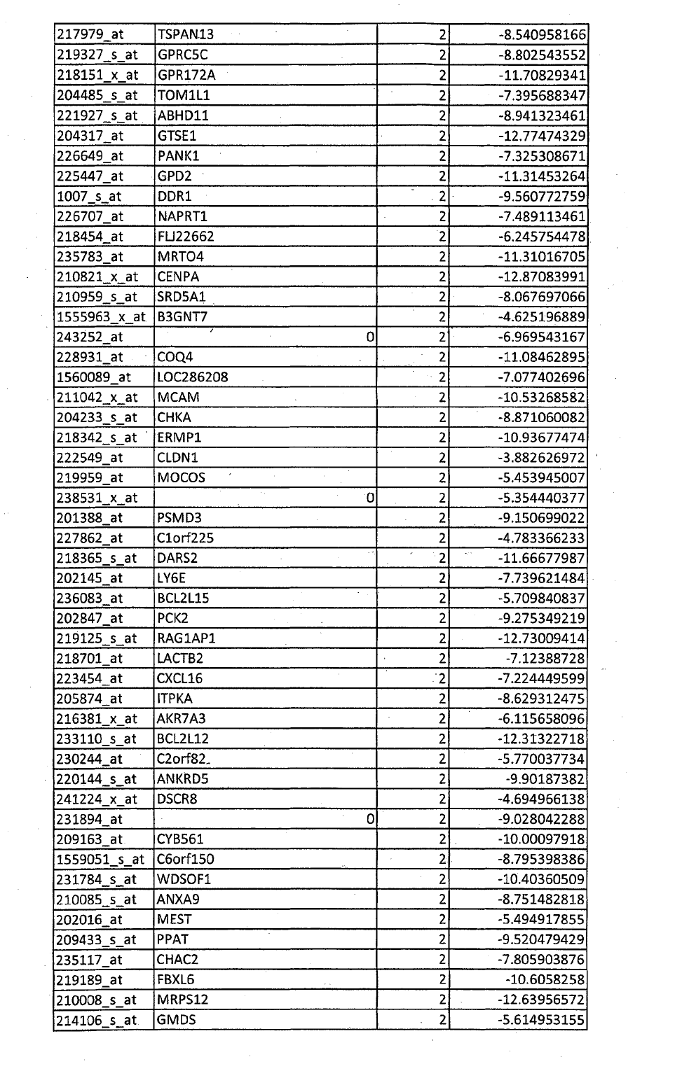

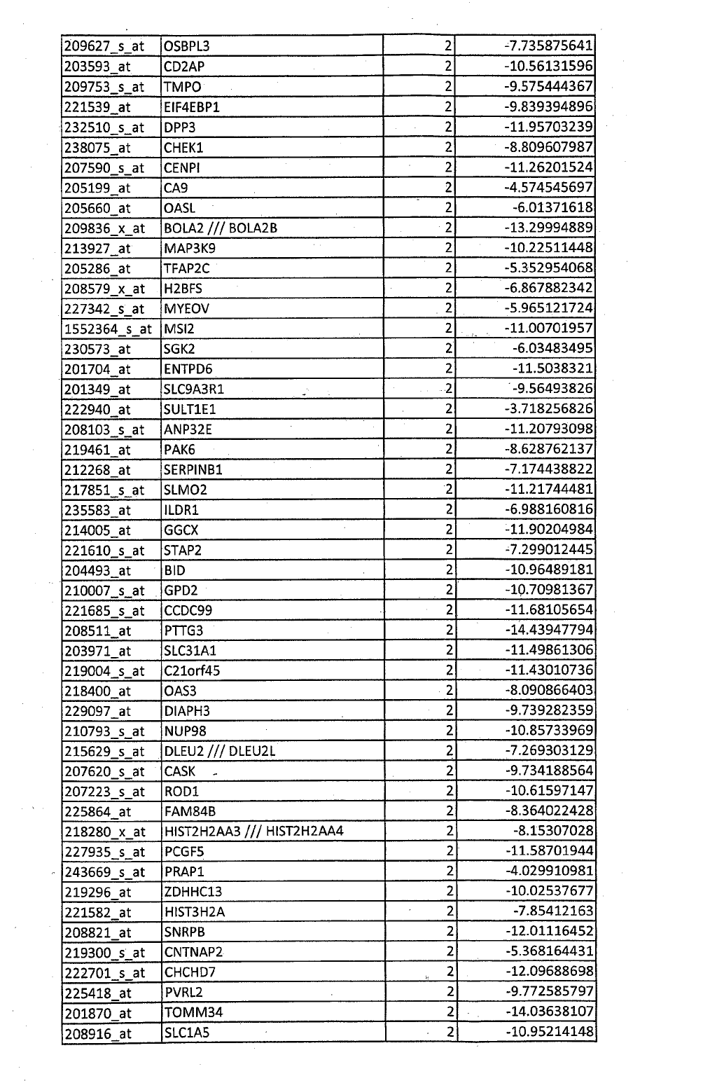

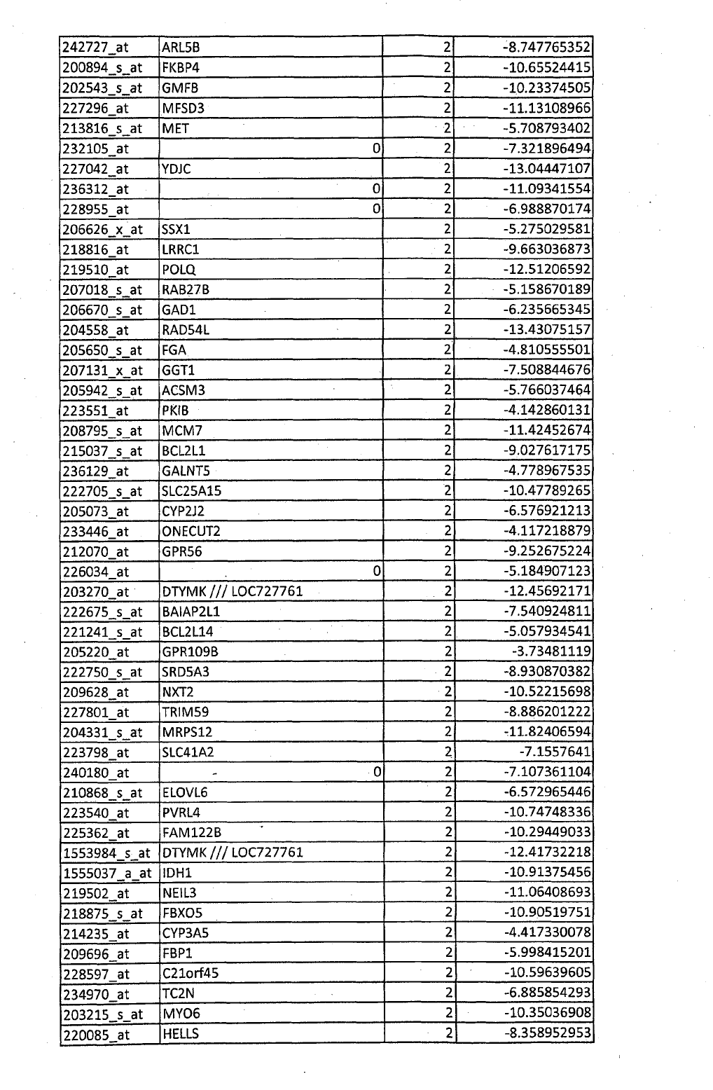

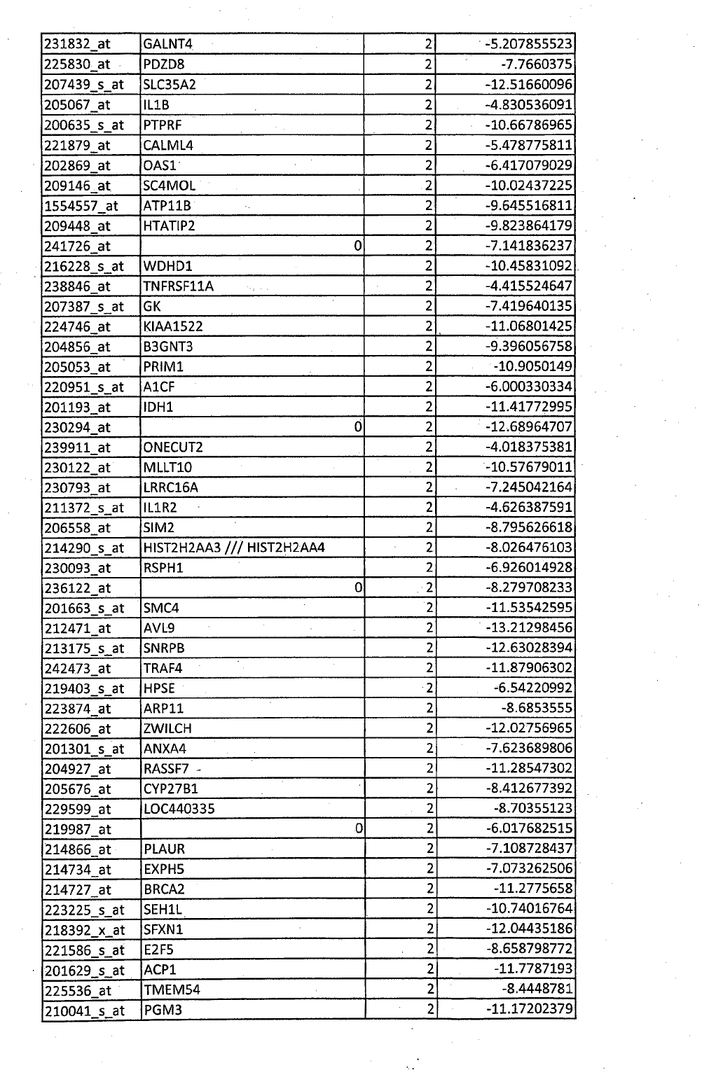

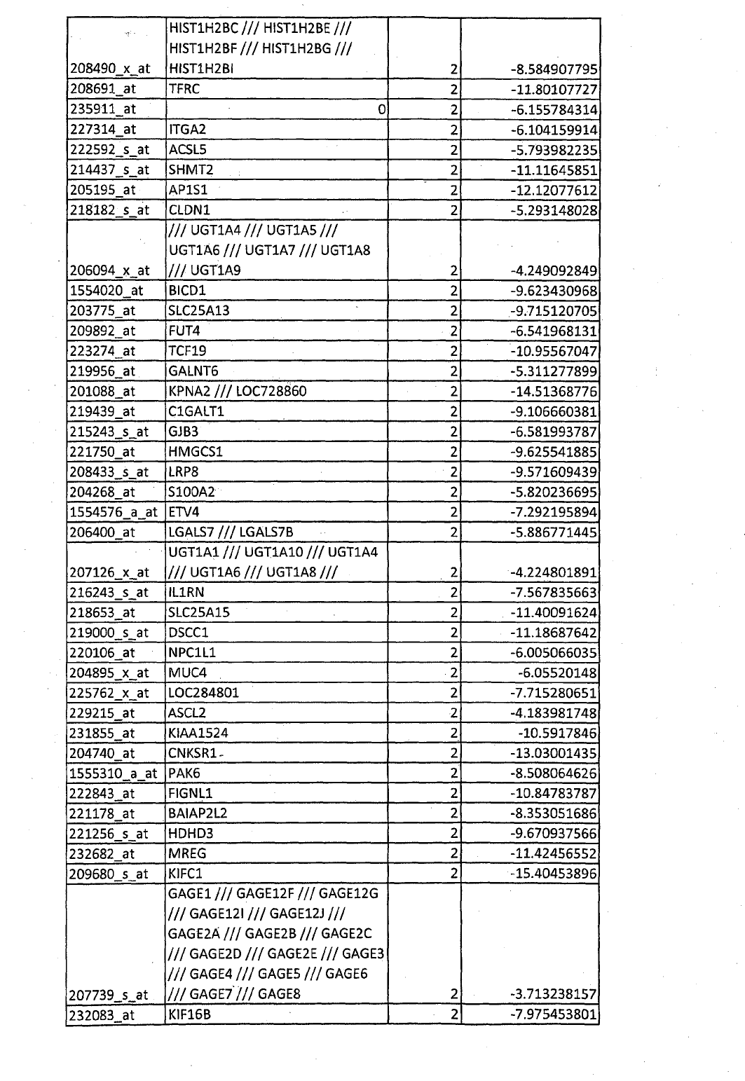

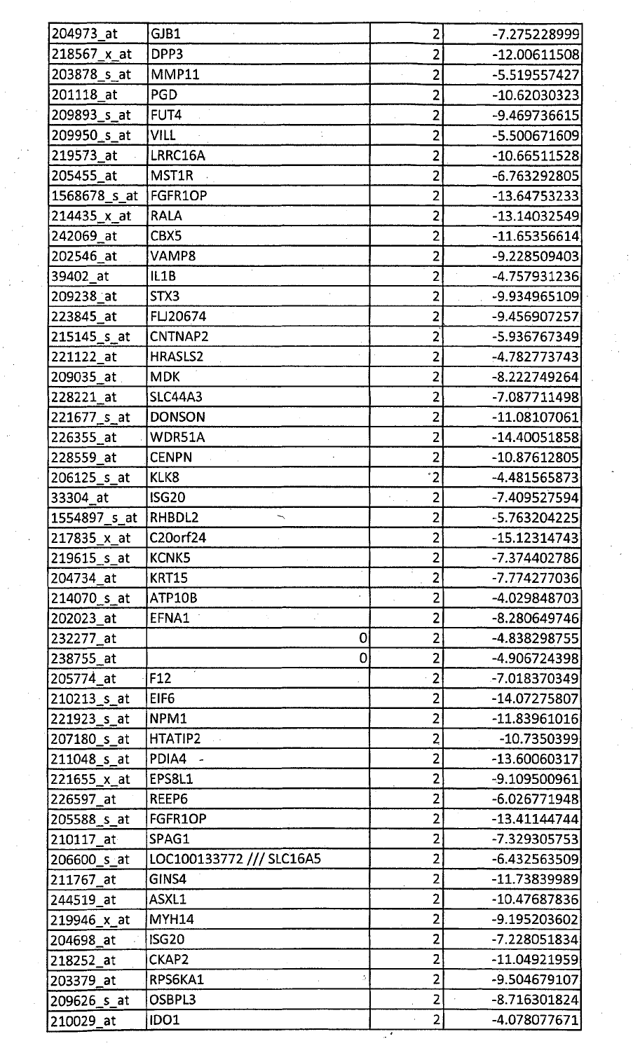

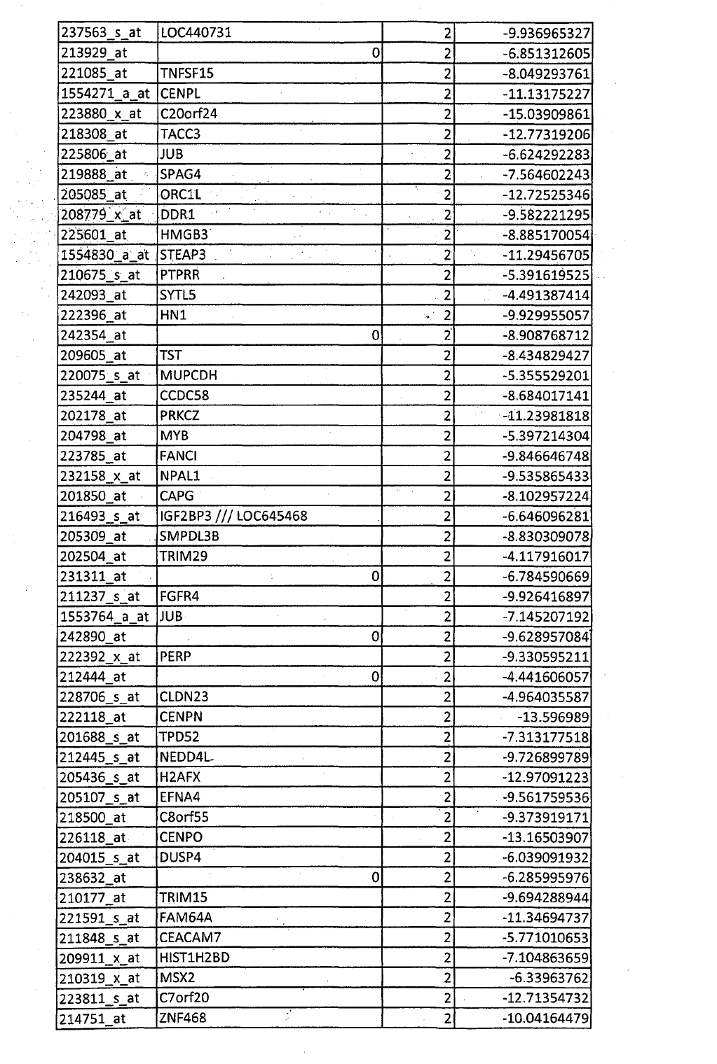

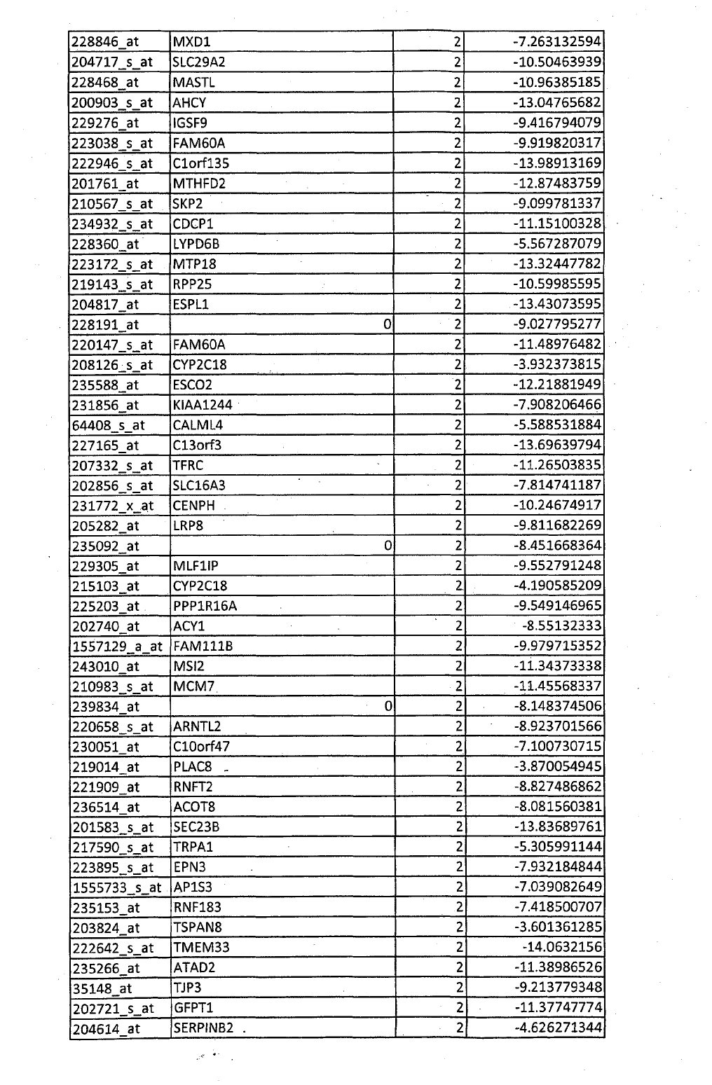

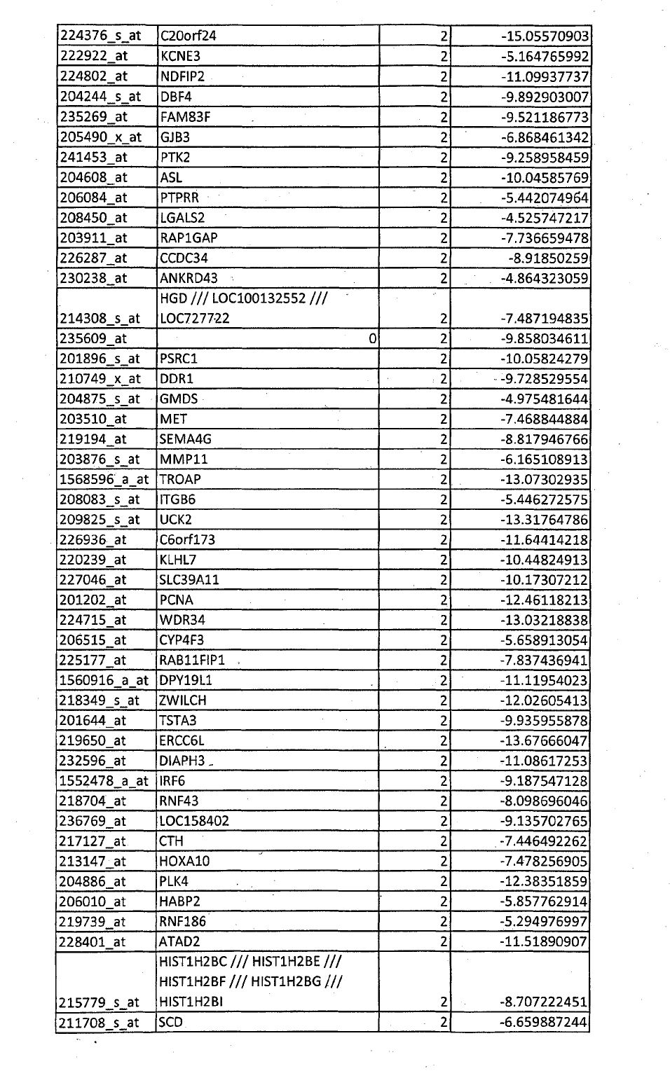

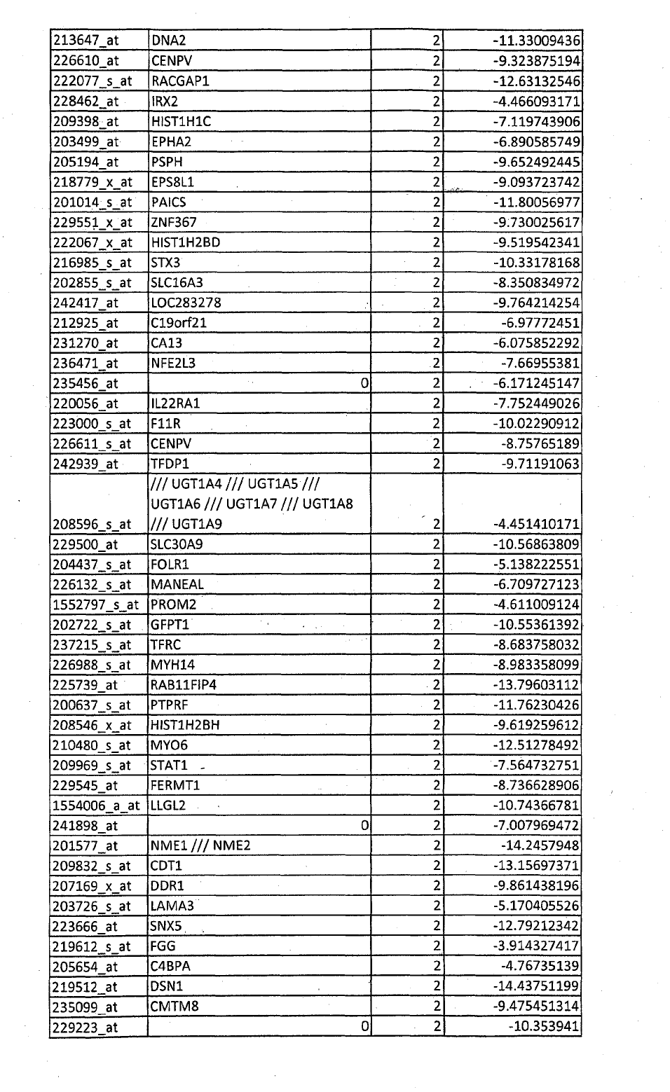

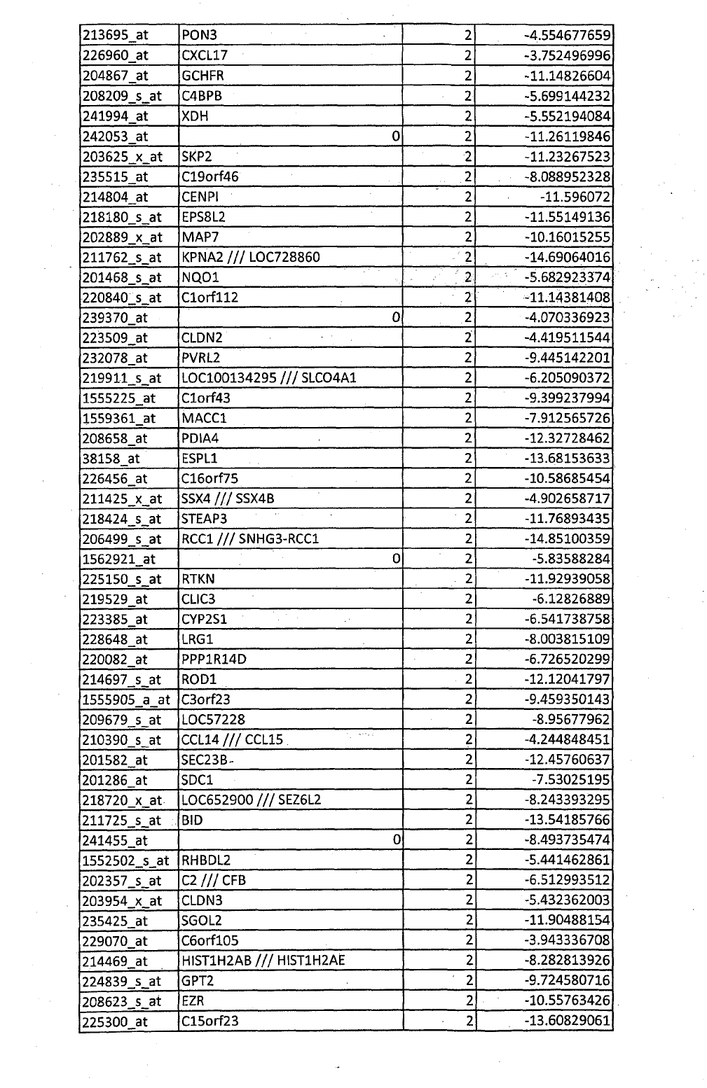

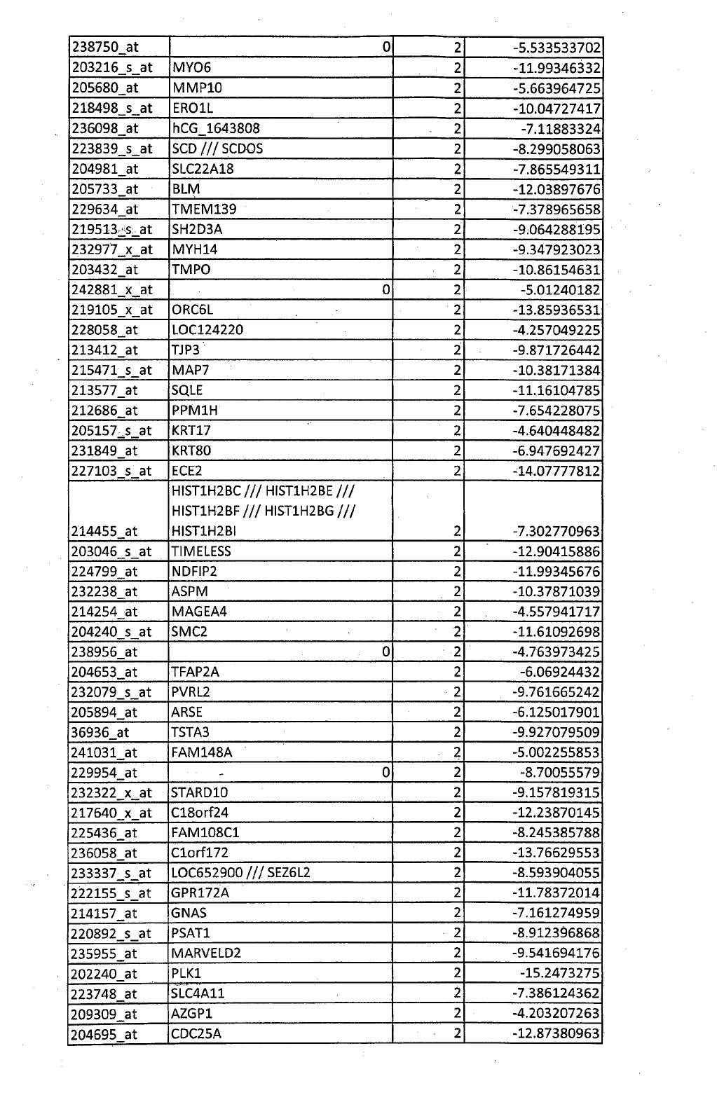

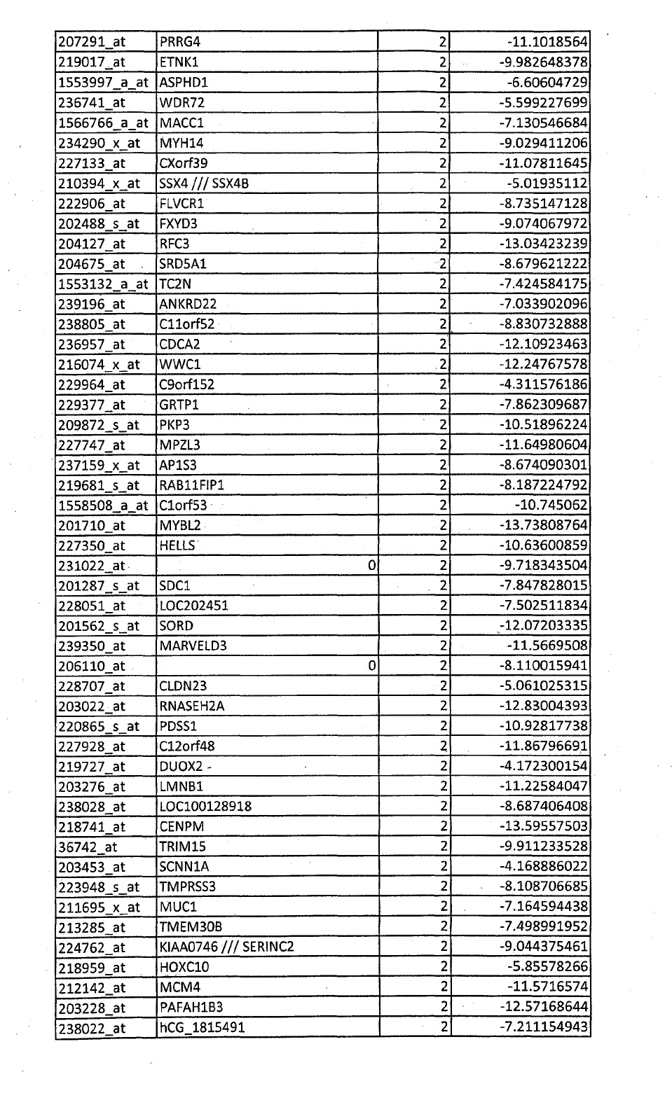

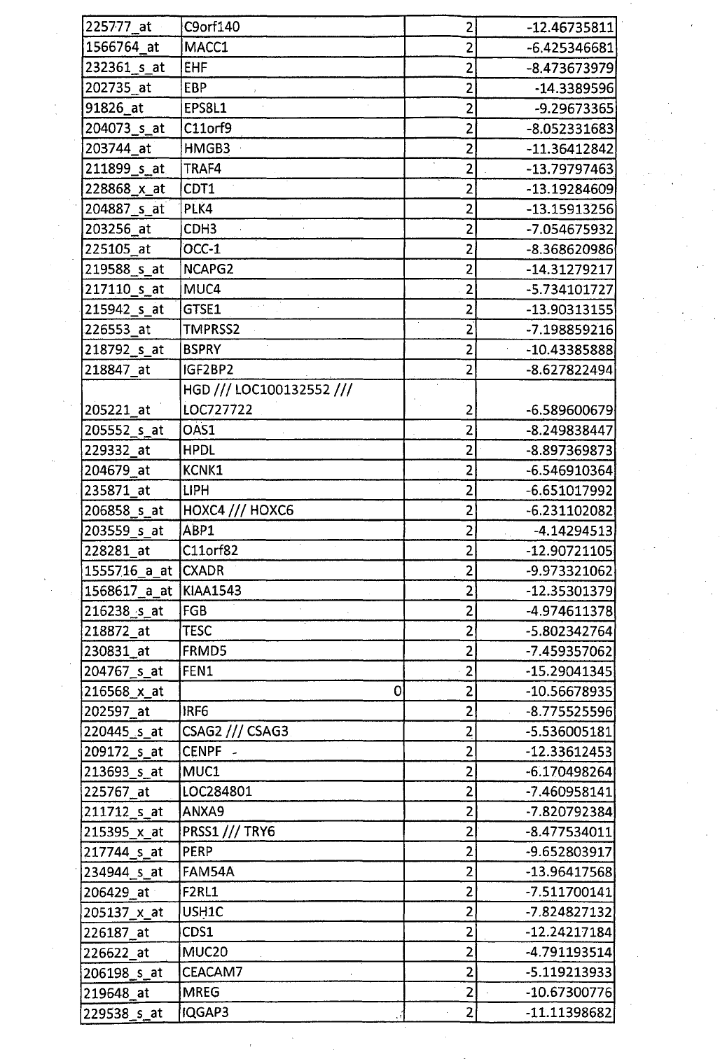

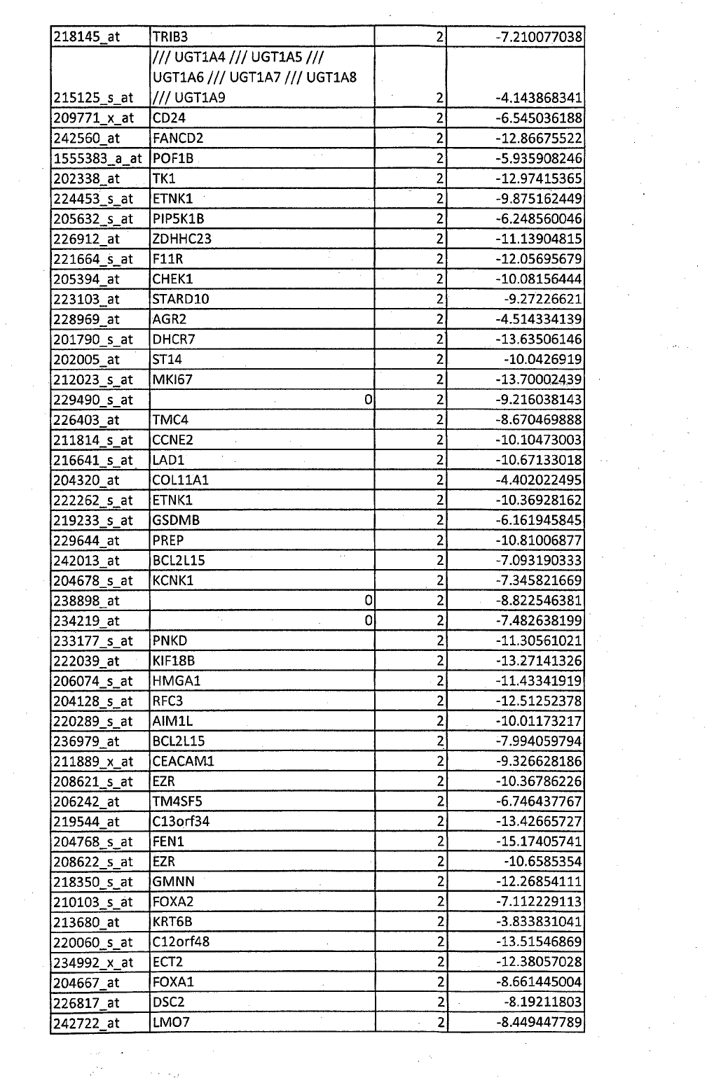

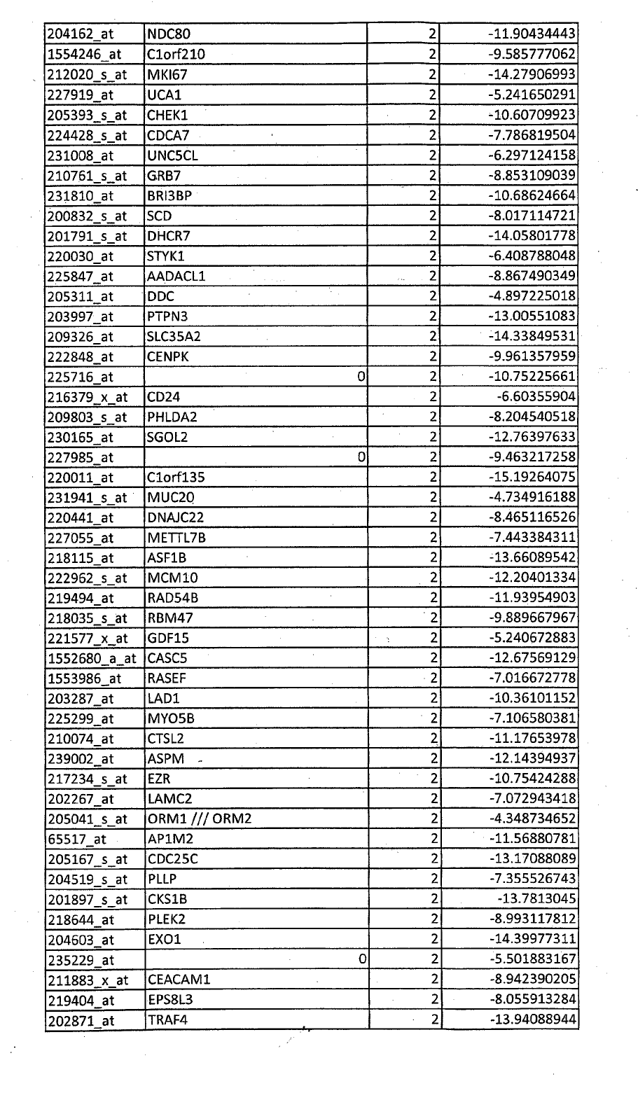

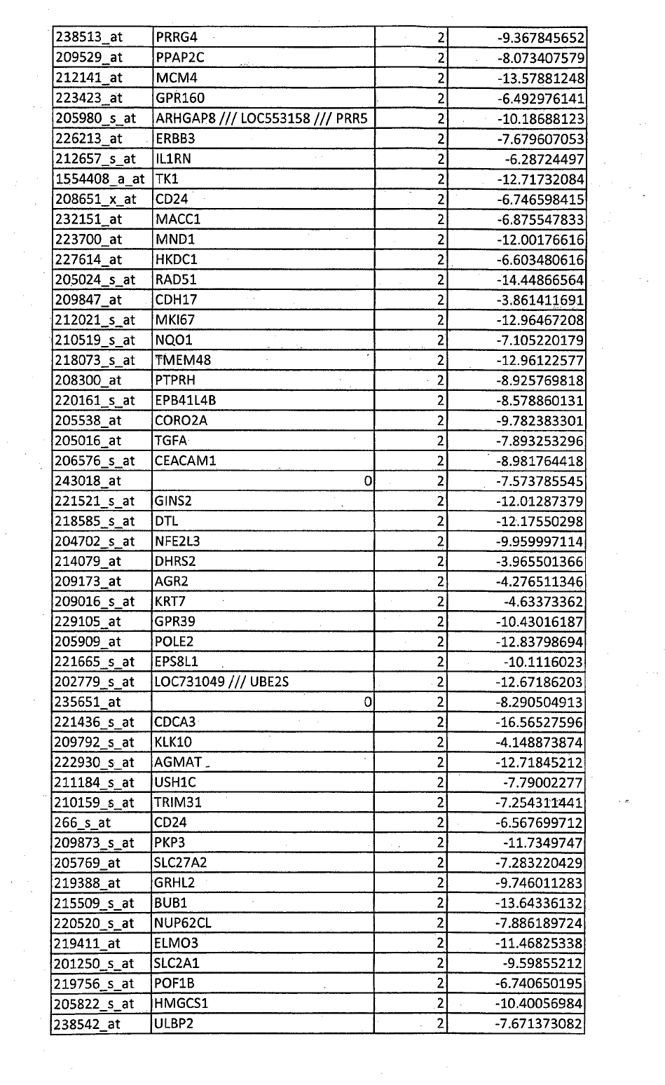

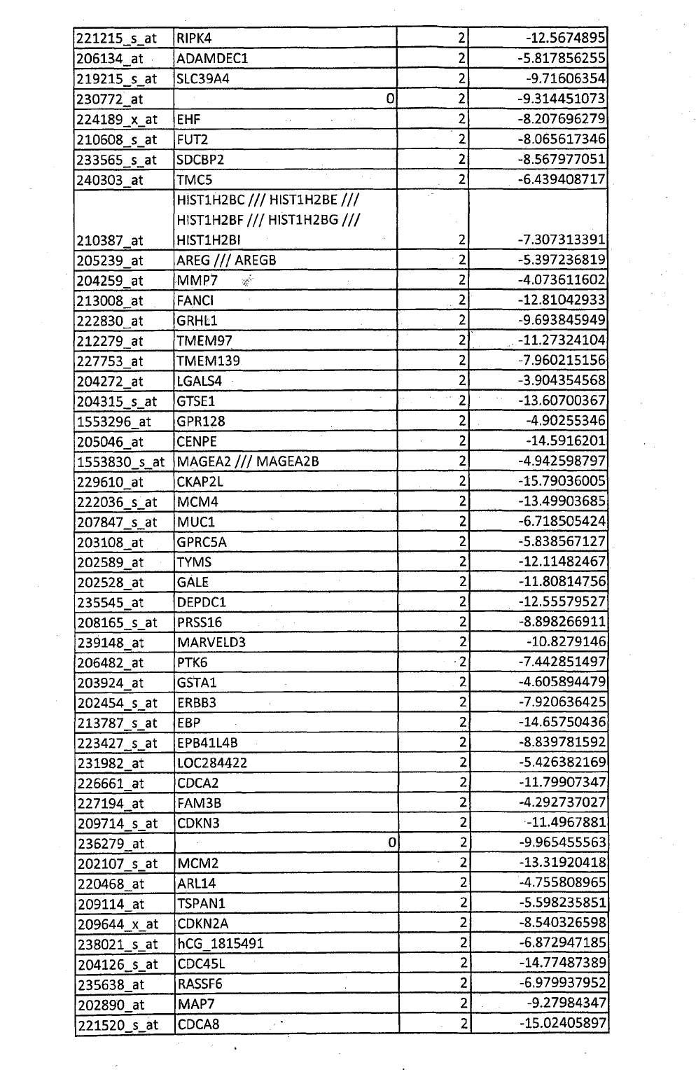

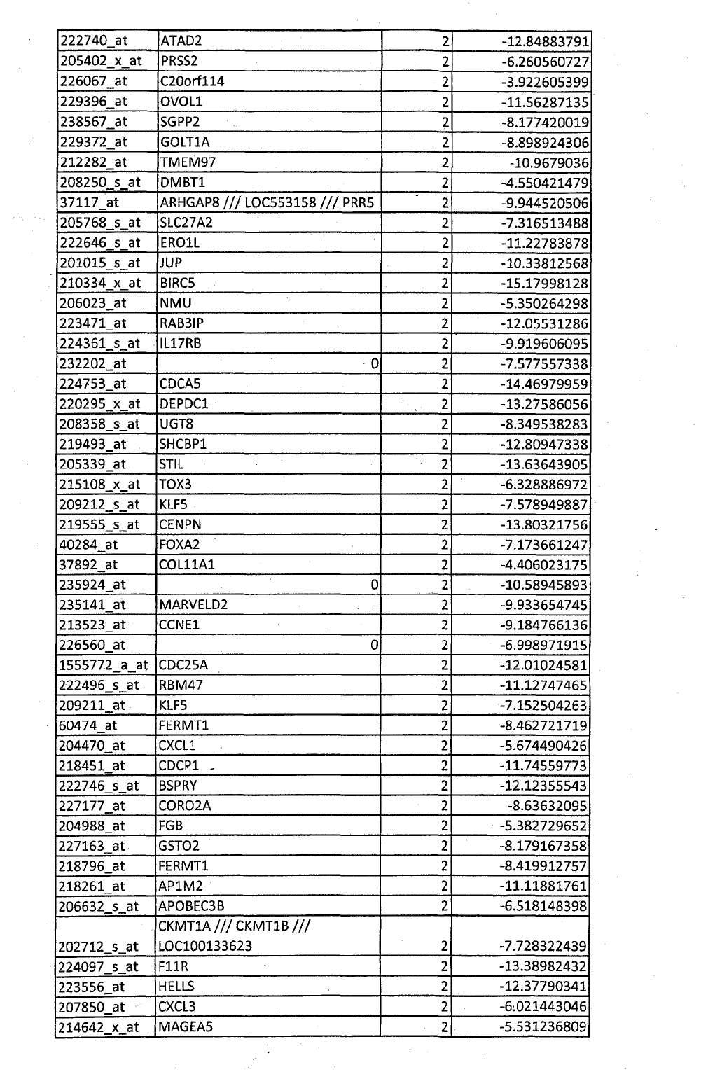

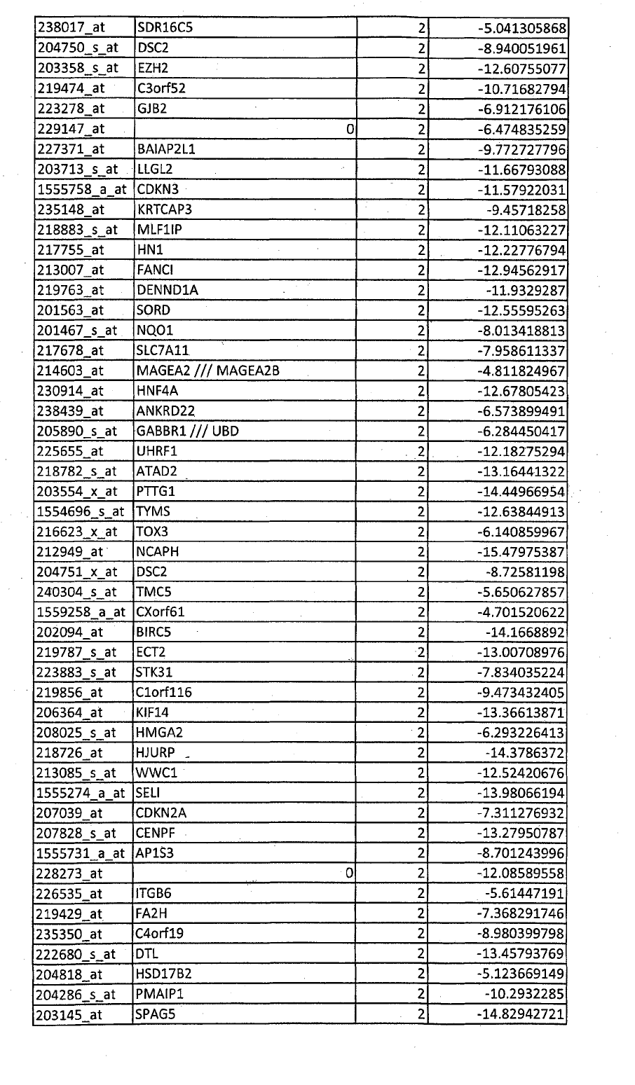

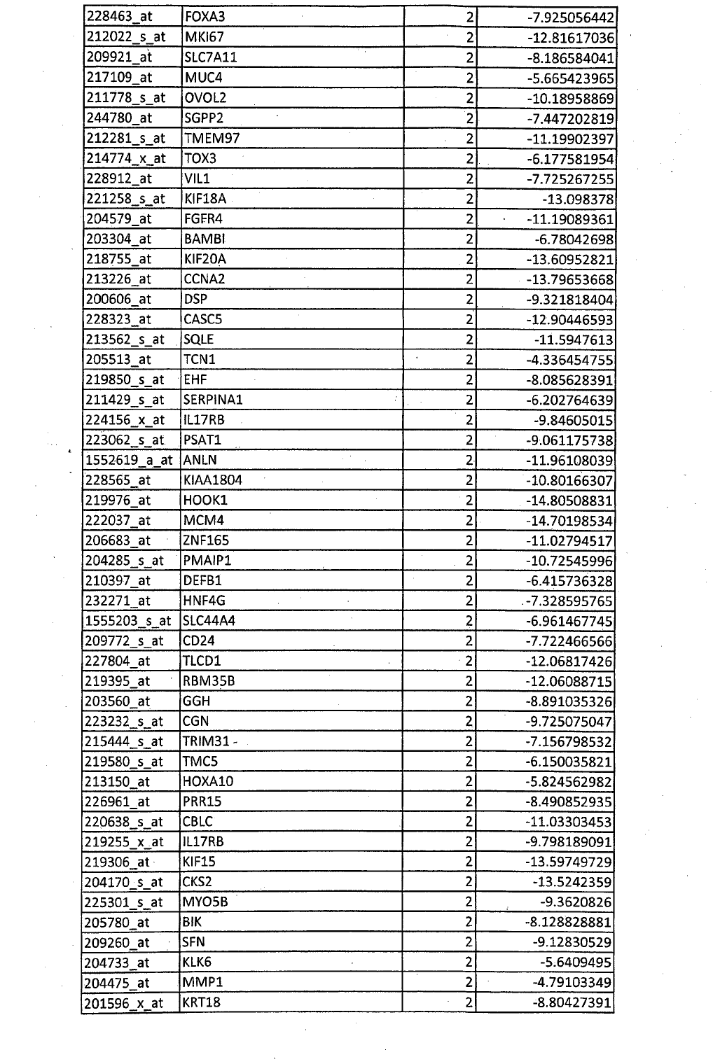

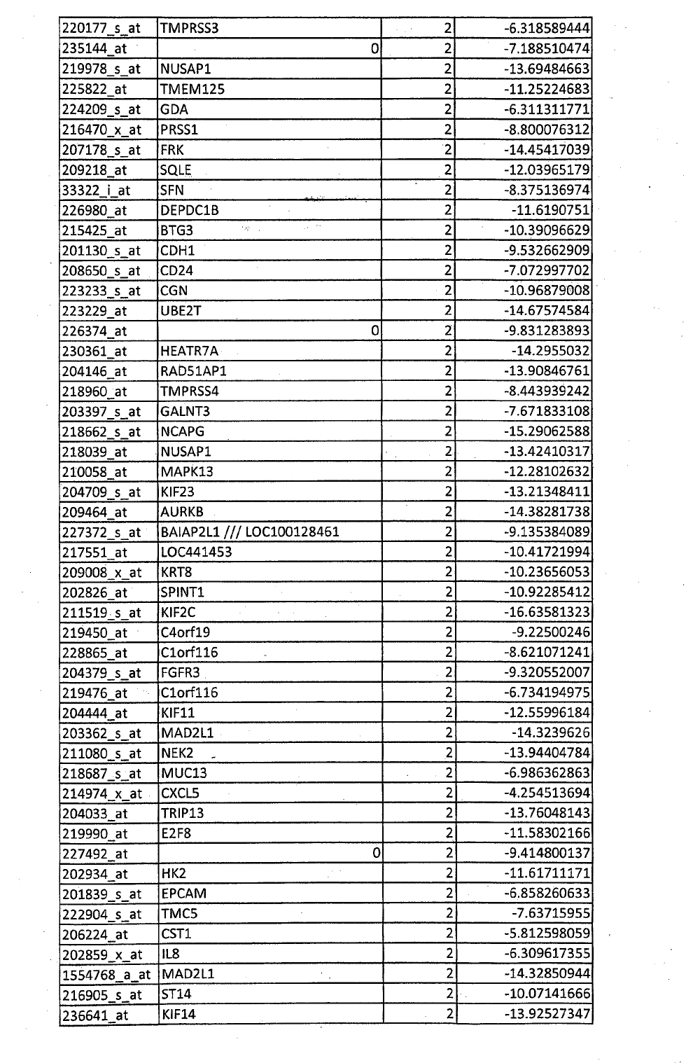

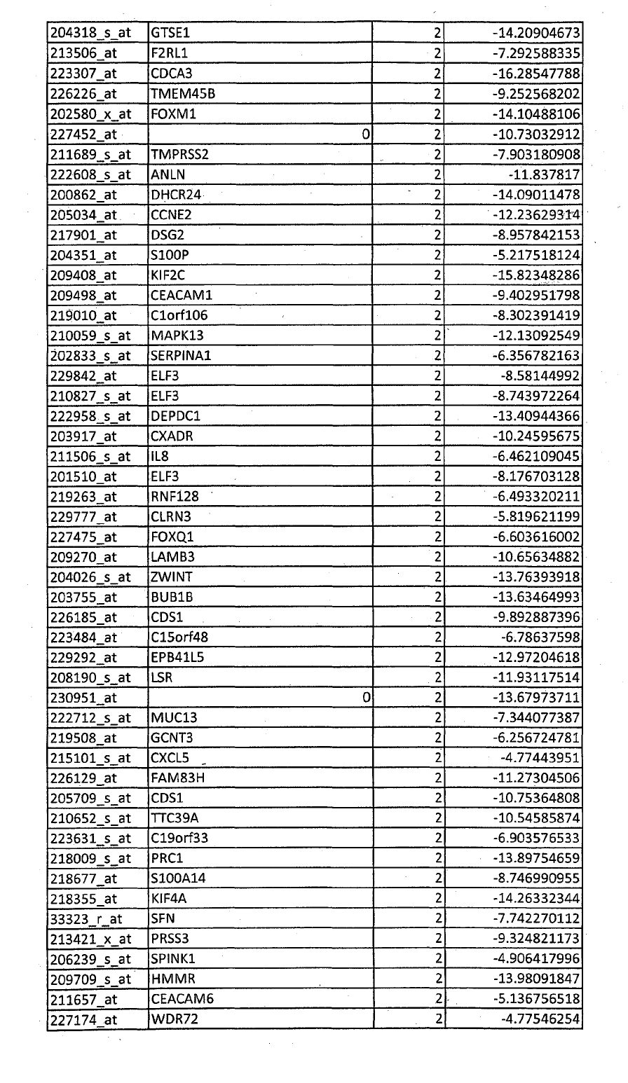

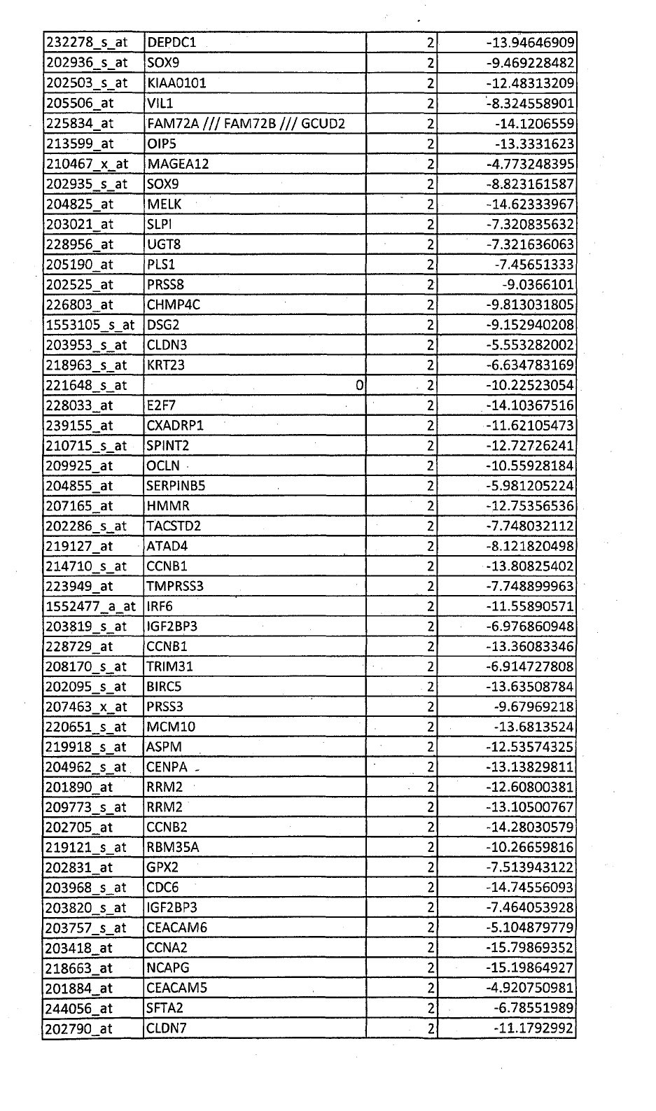

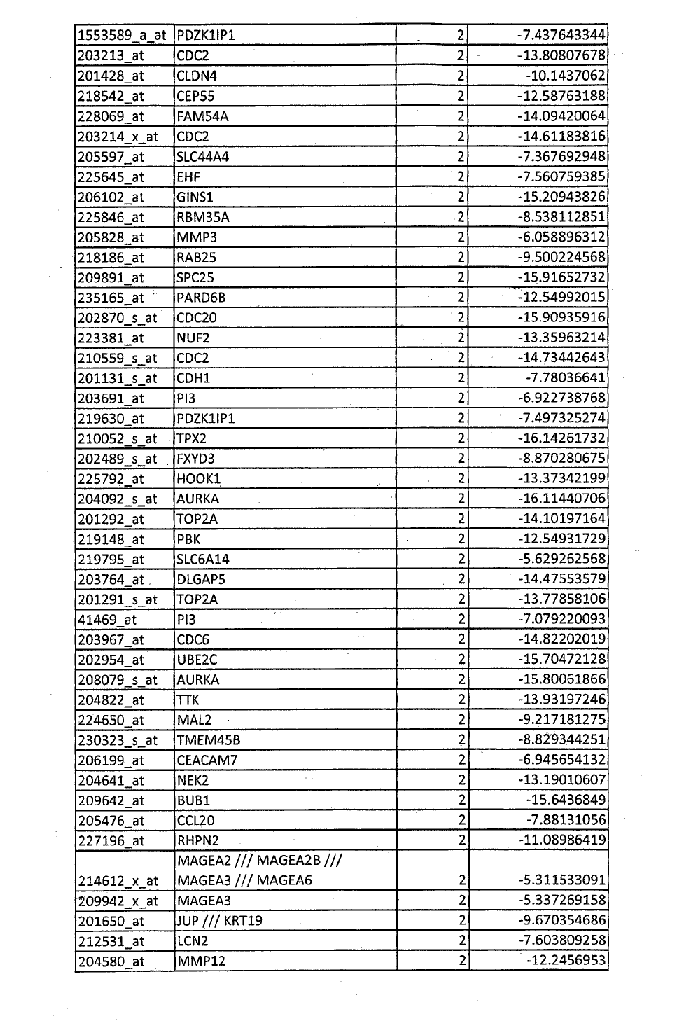

11. The predictor according to claim 8, wherein a first of the three predictors comprises a differentially expressed gene set comparing the differential expression between genes of the invasive subtype versus the proliferative subtype as shown in FIG. 18.

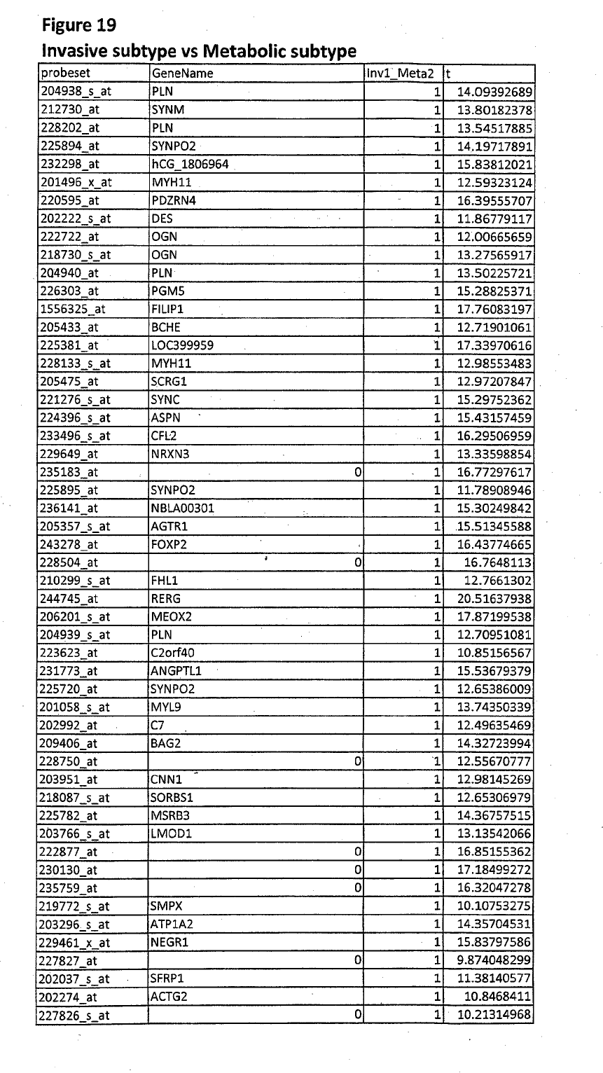

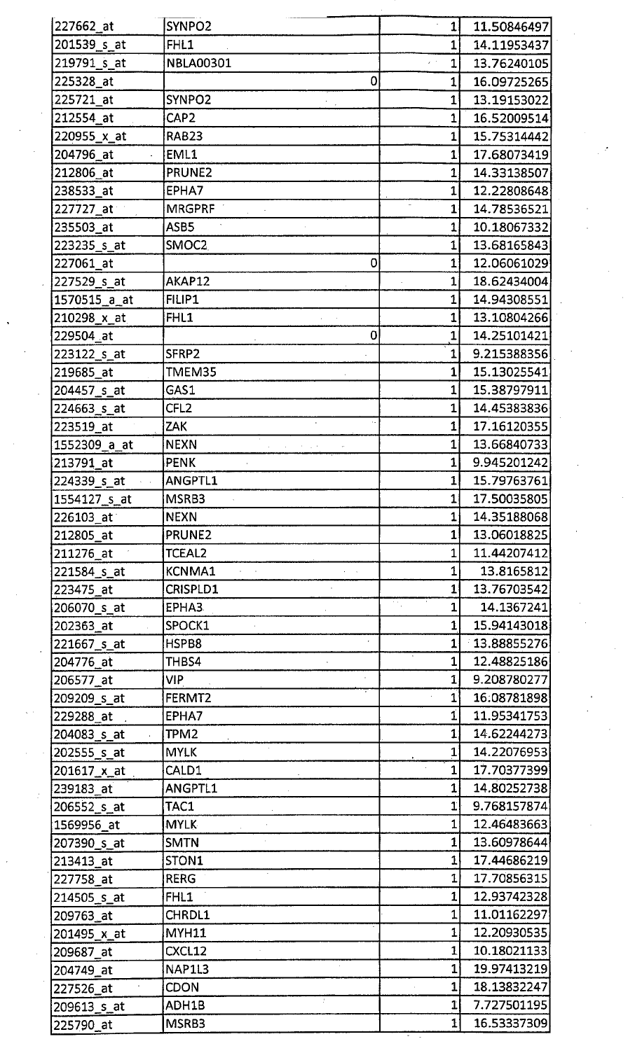

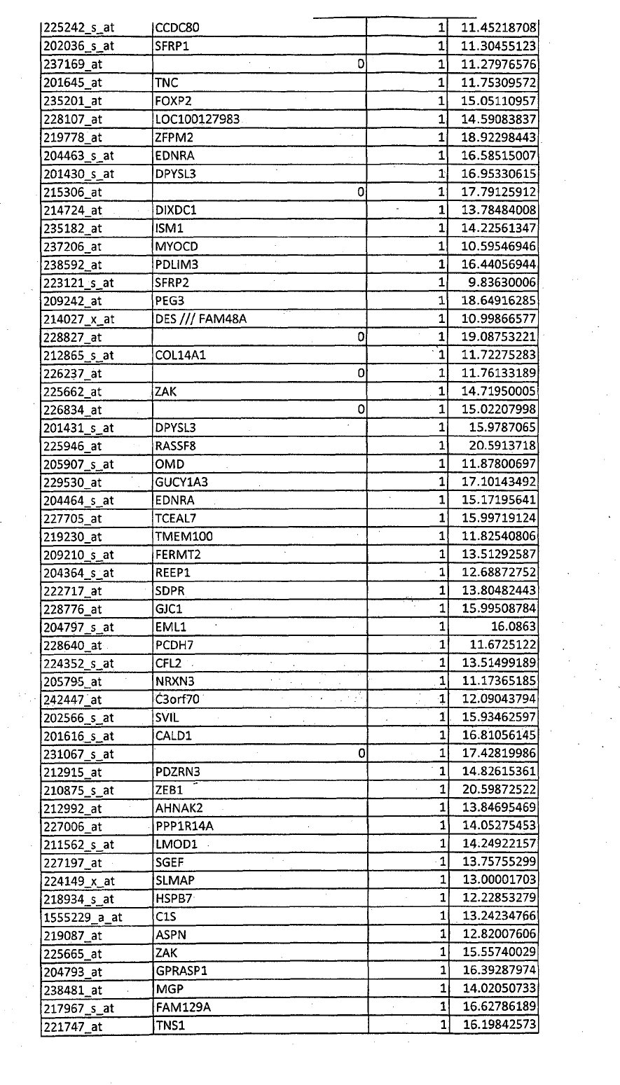

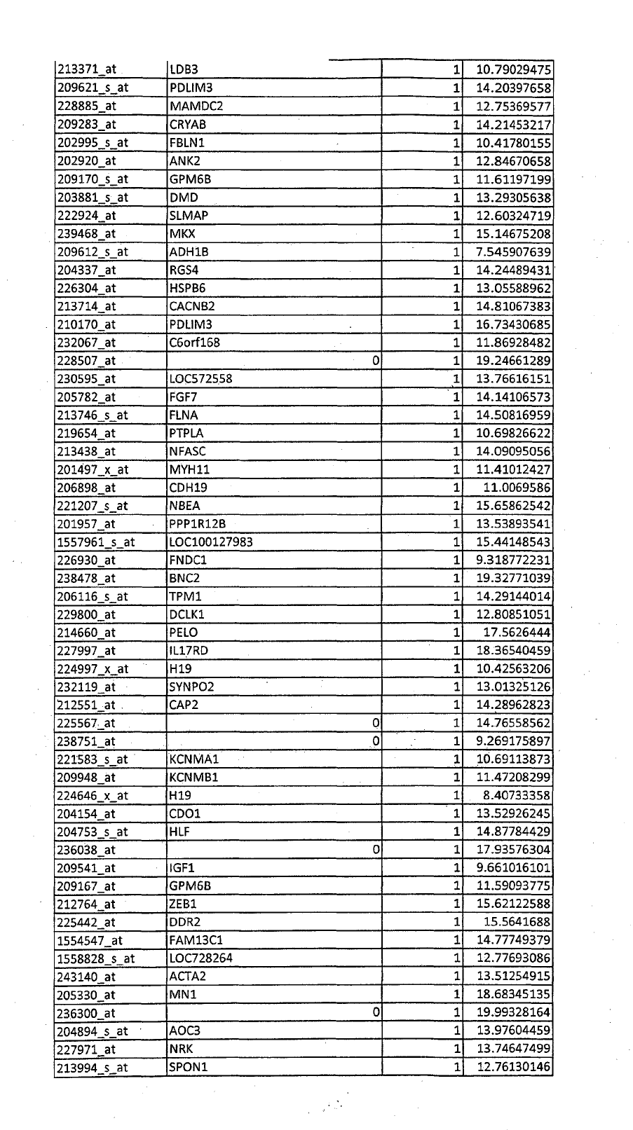

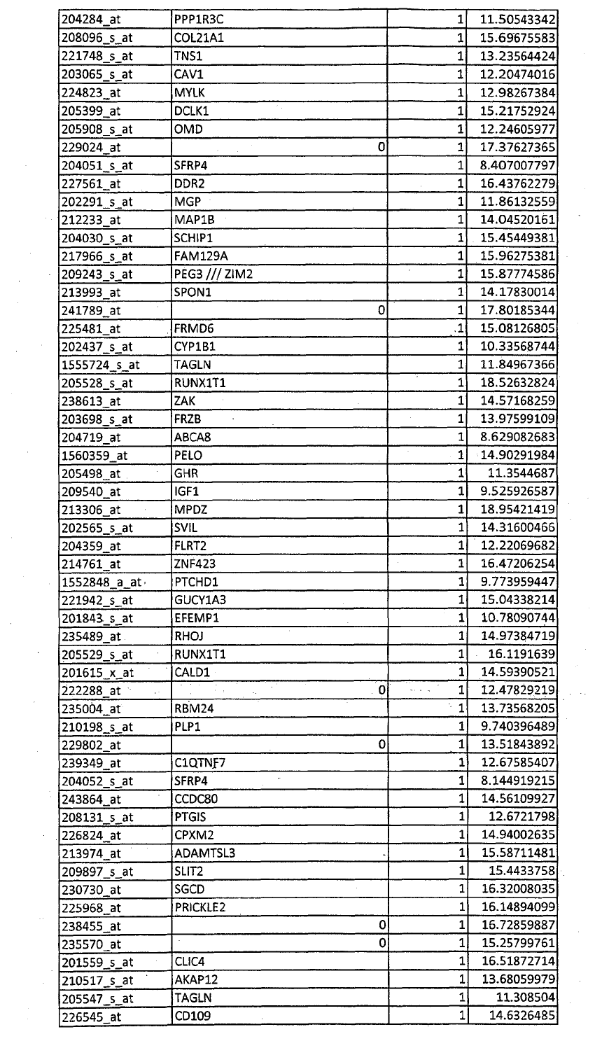

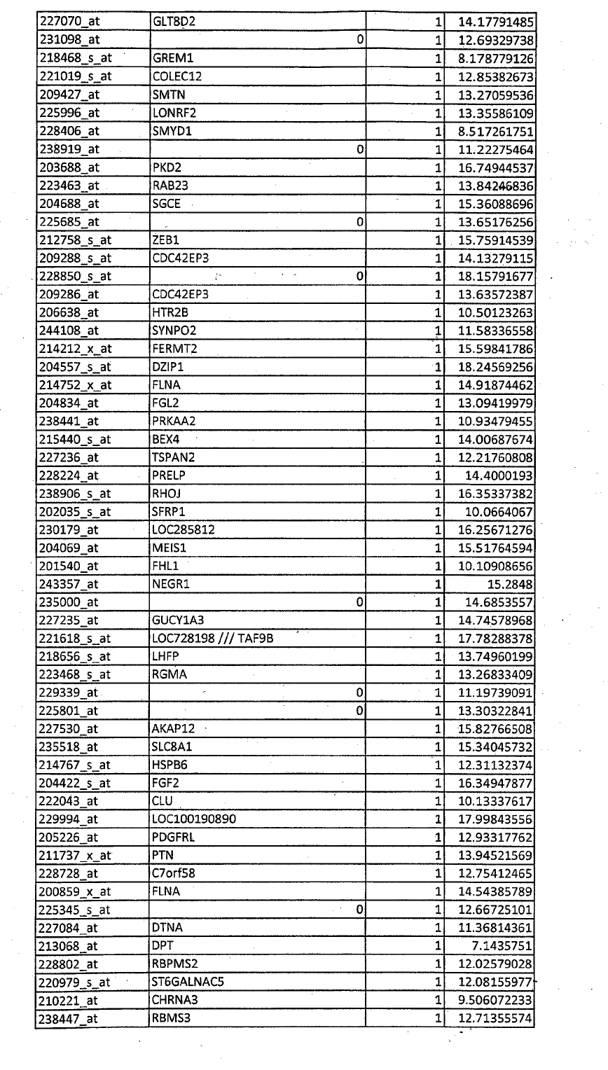

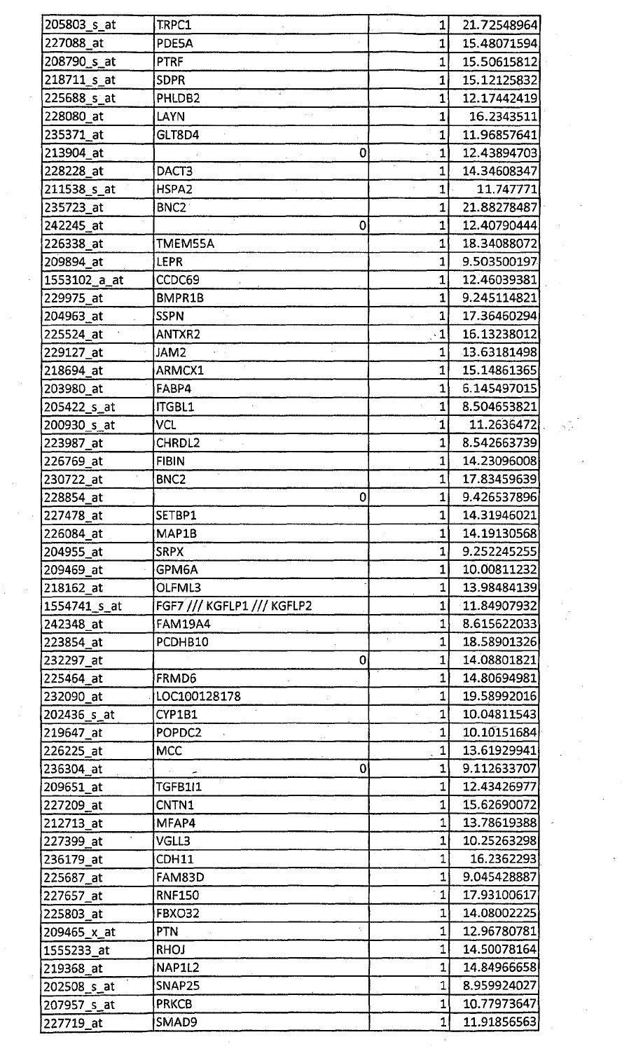

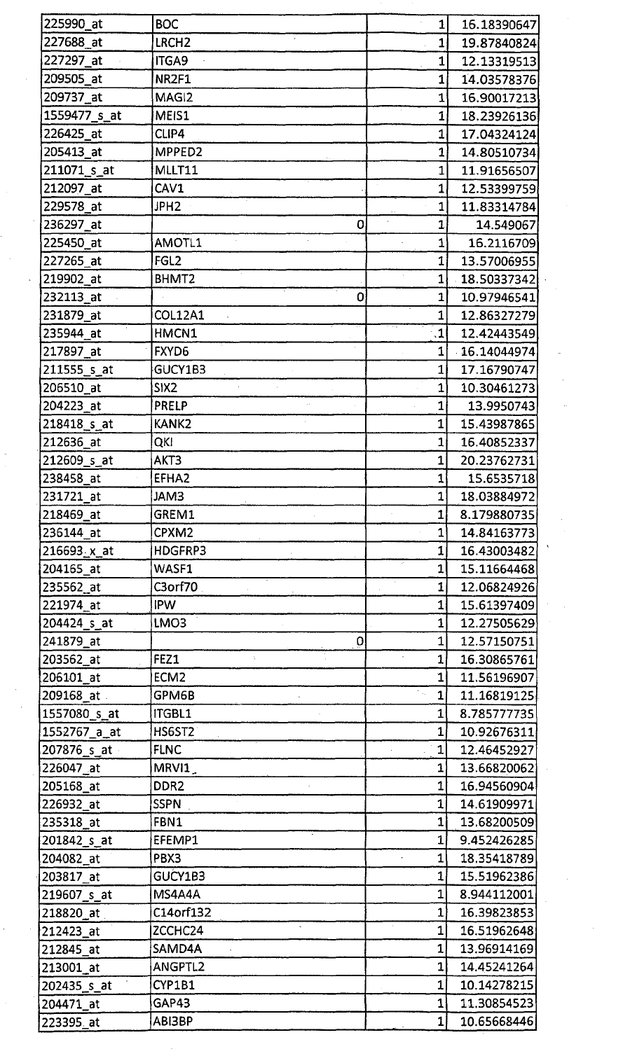

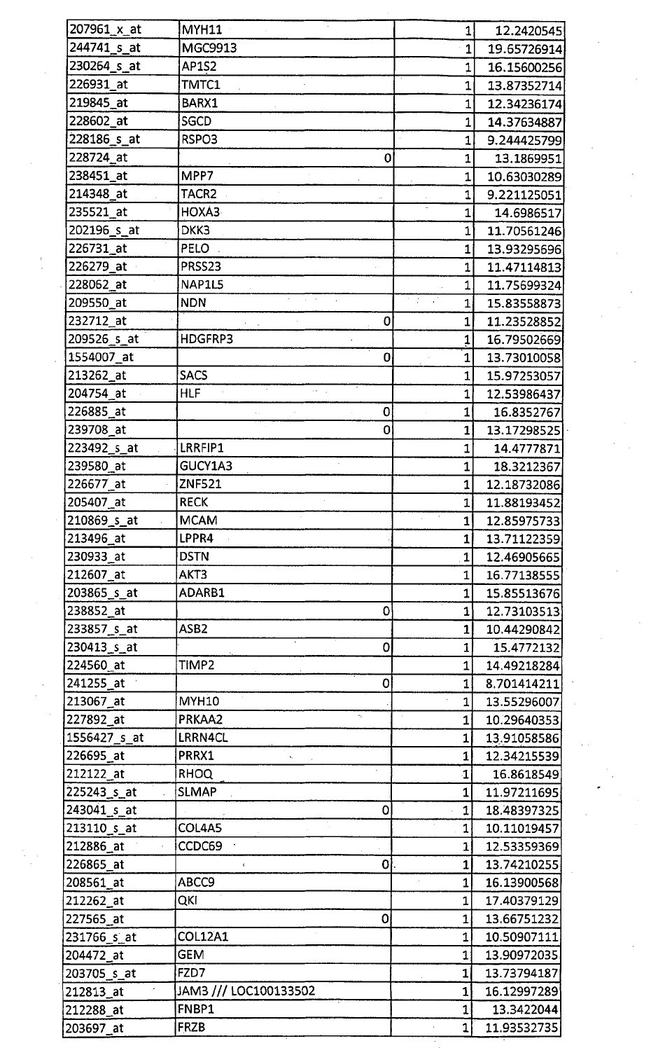

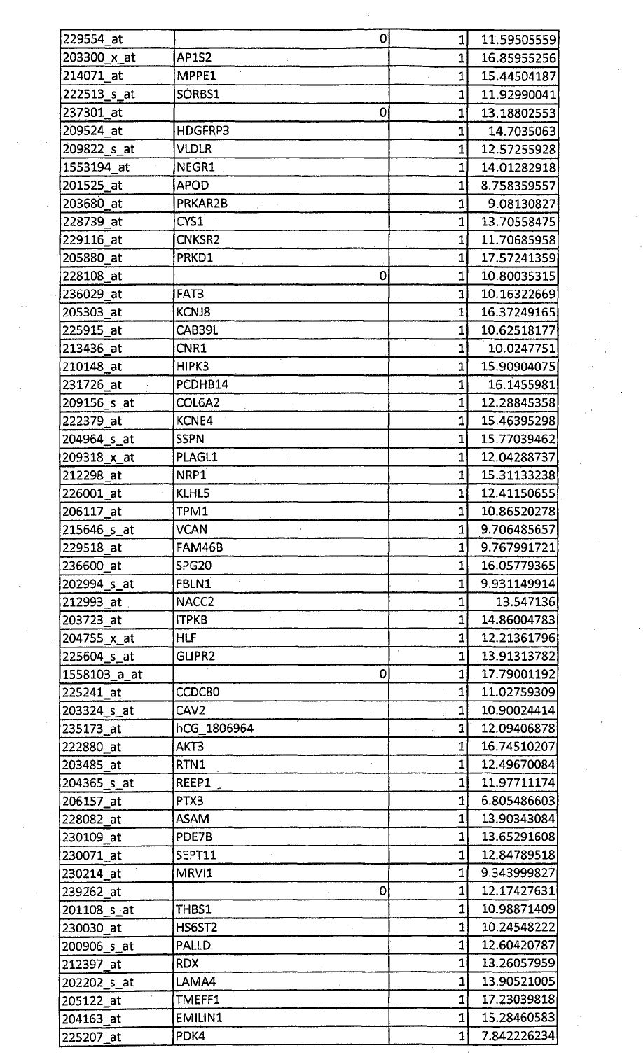

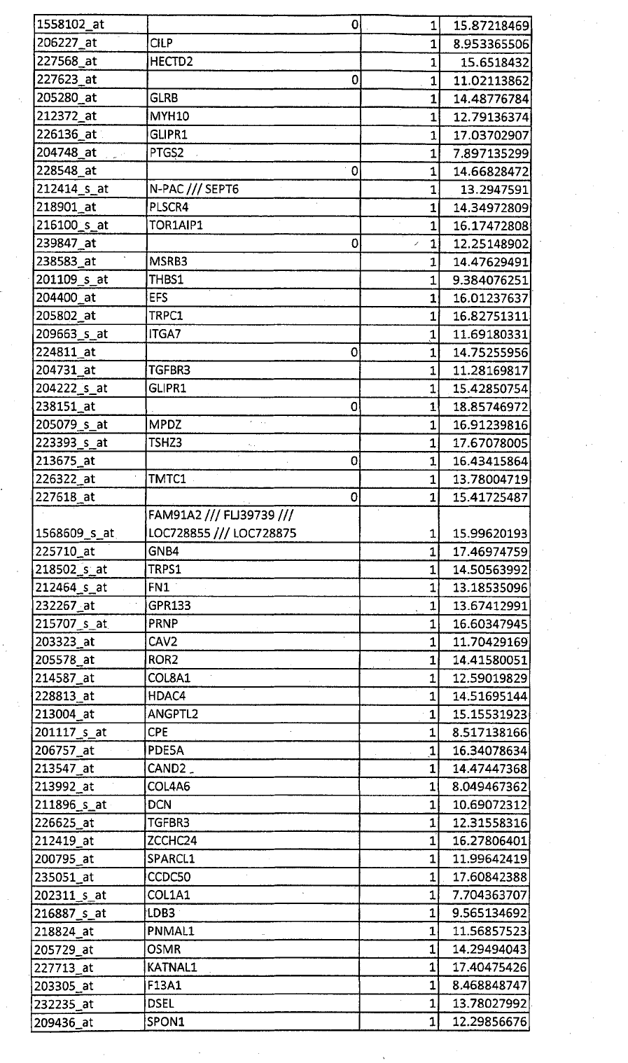

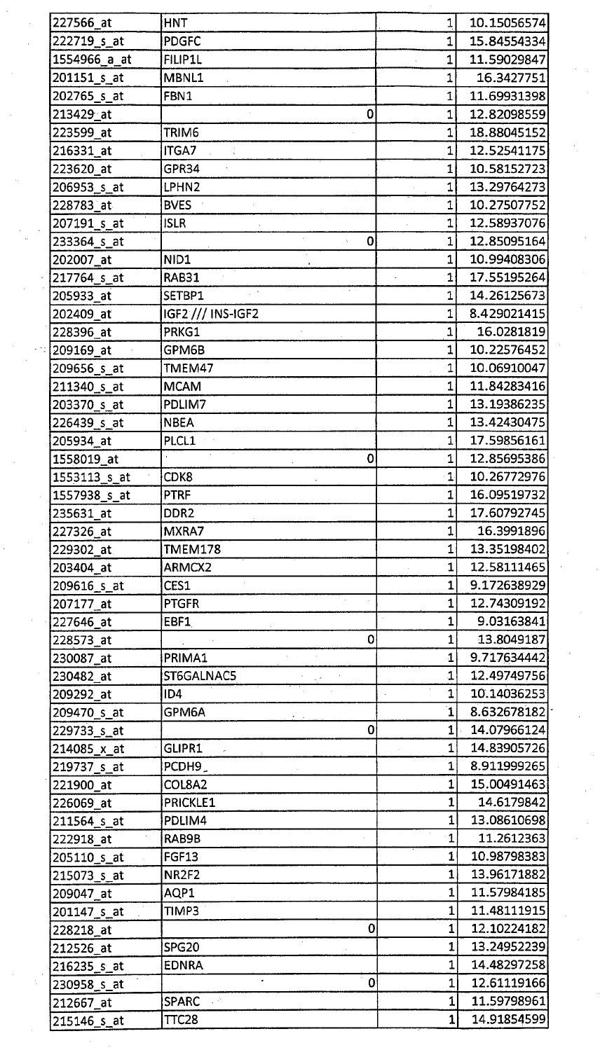

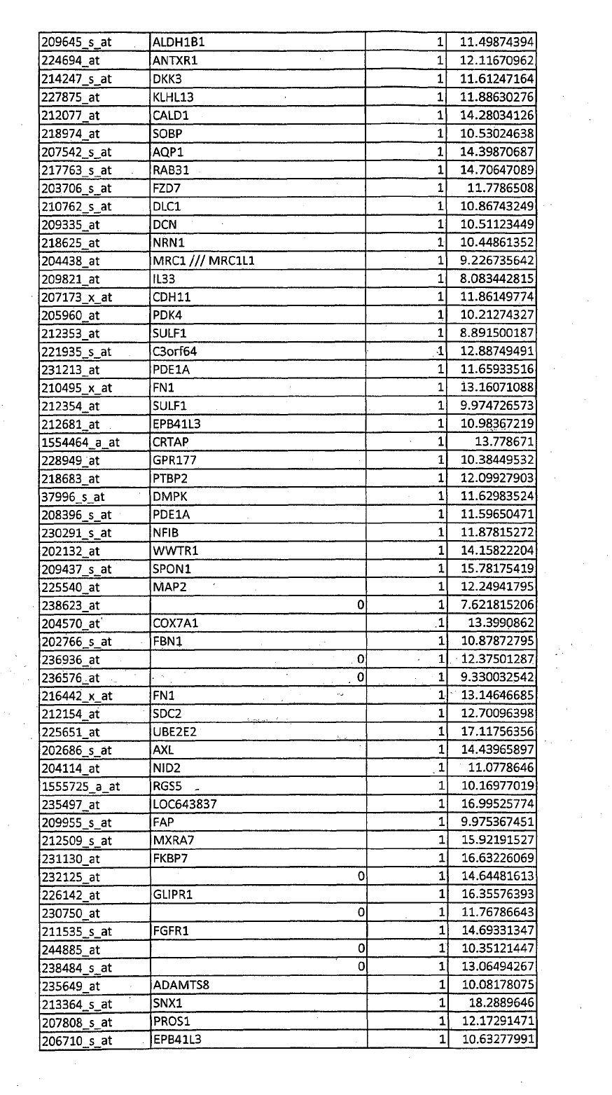

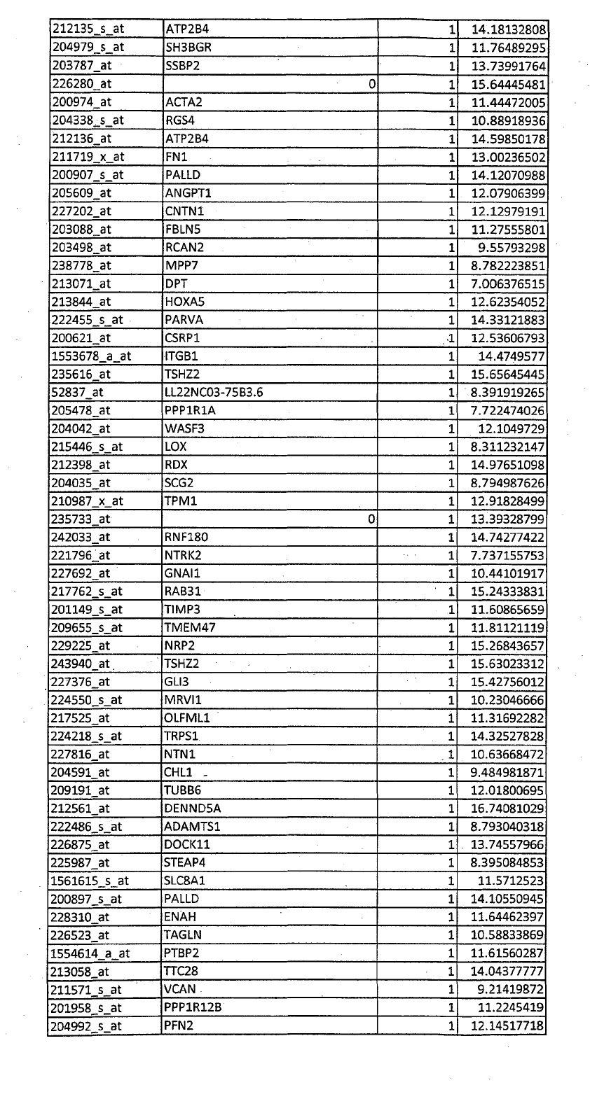

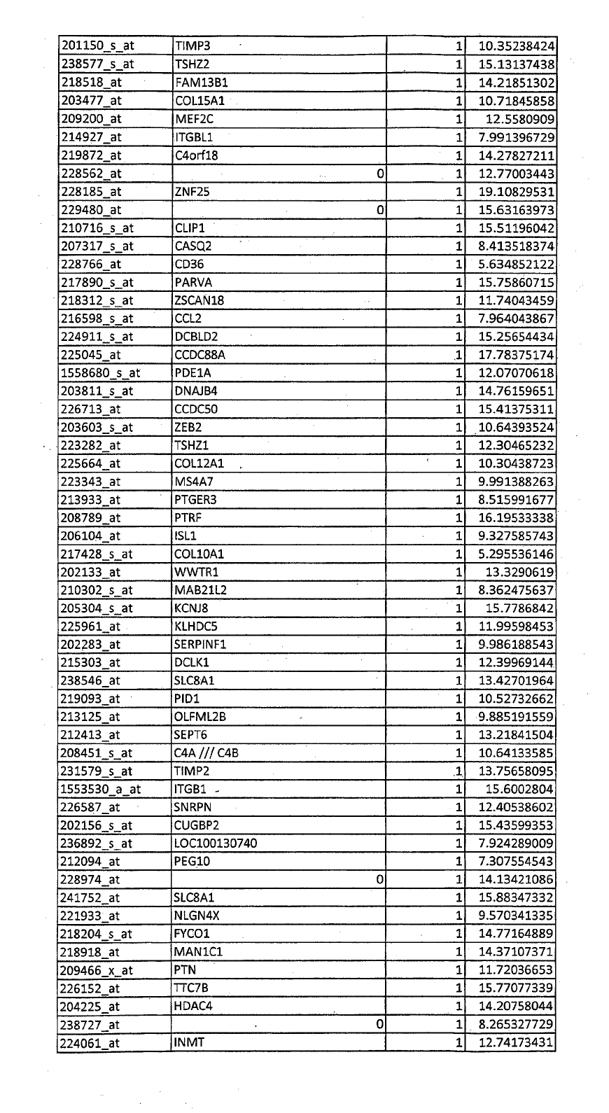

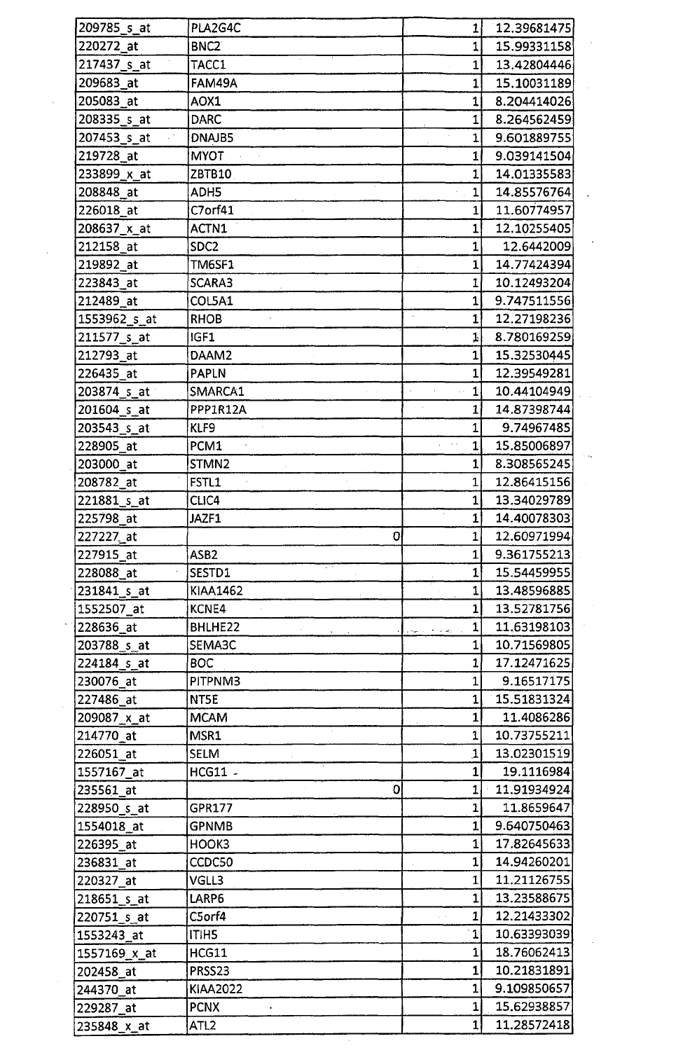

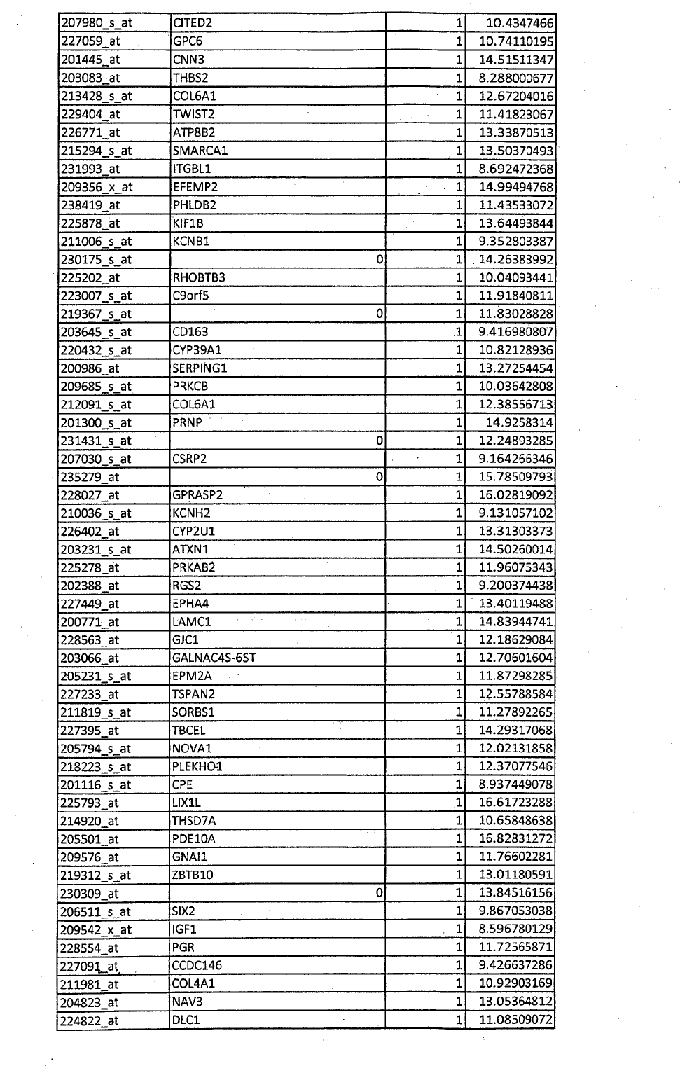

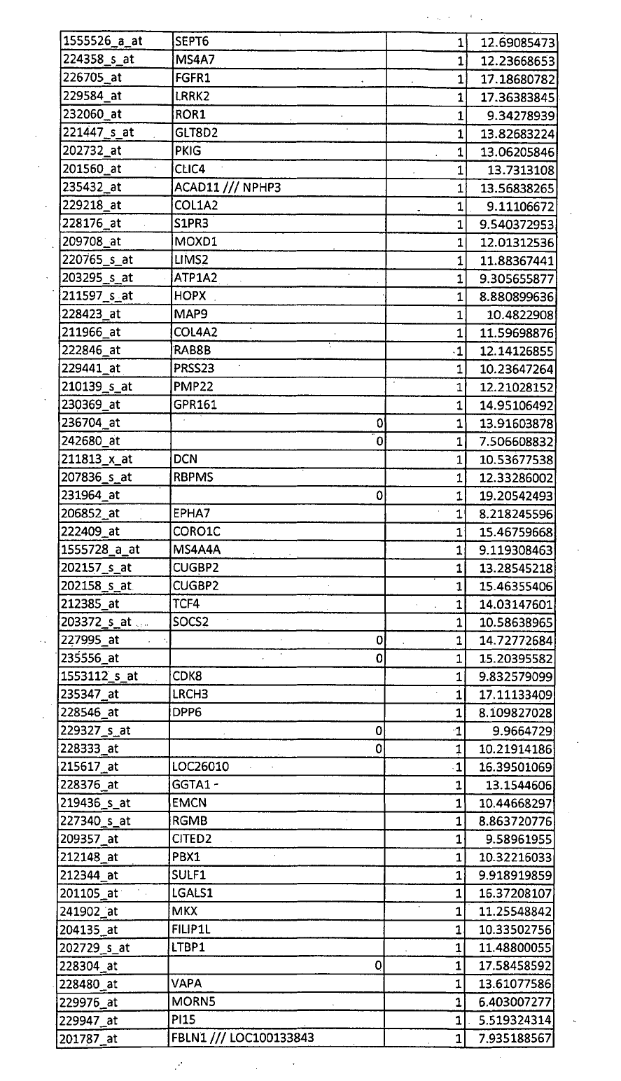

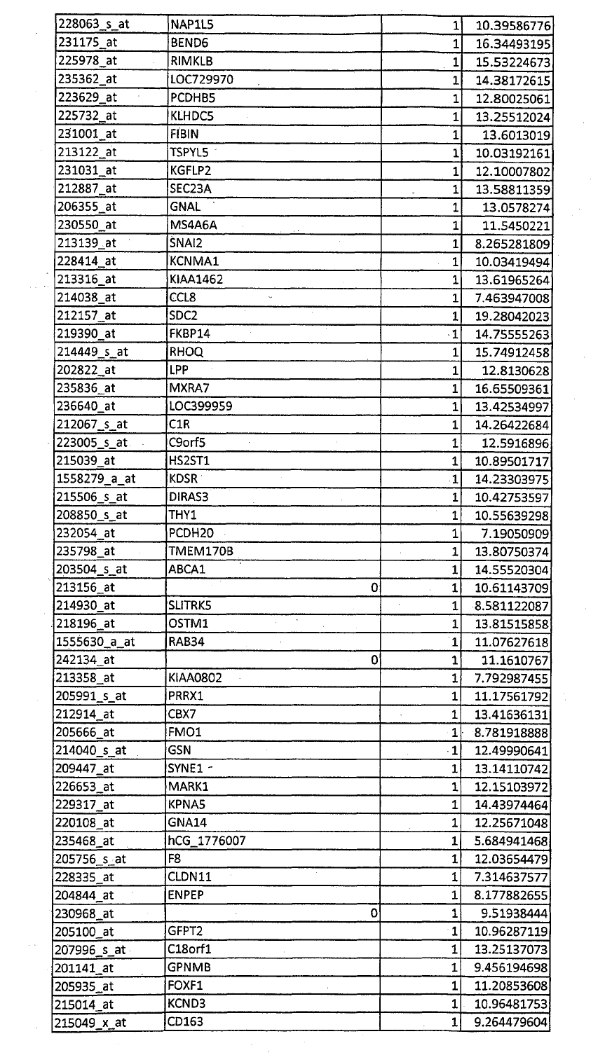

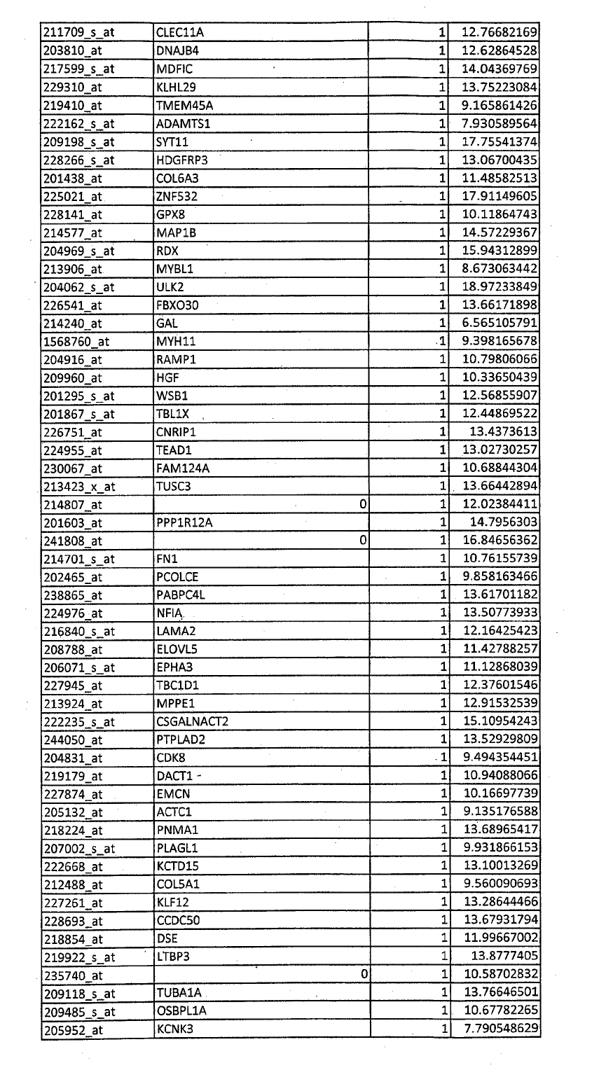

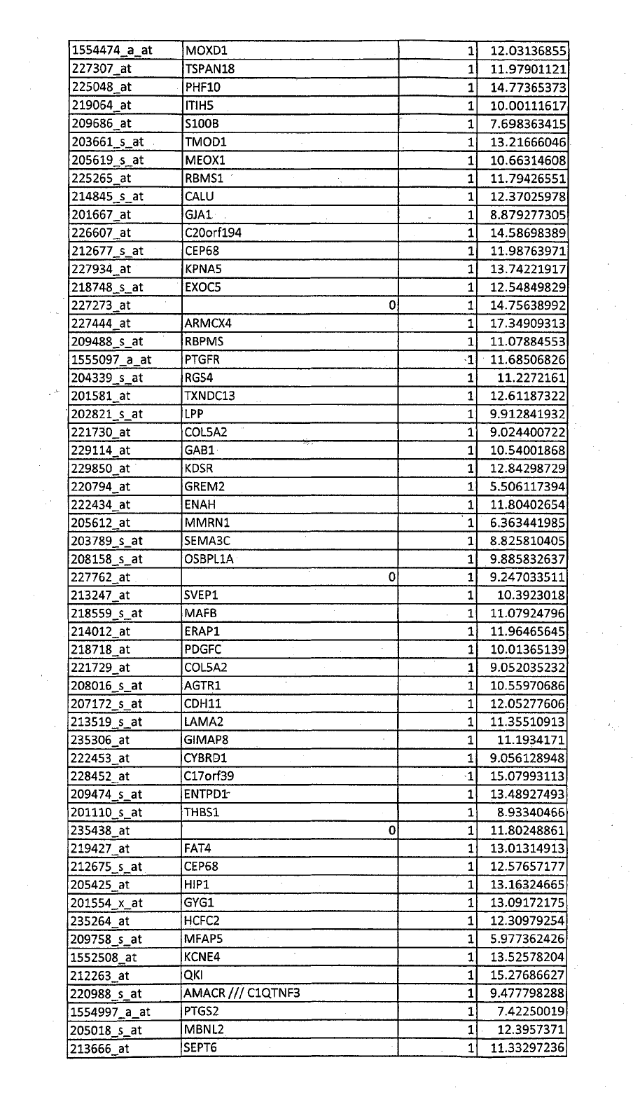

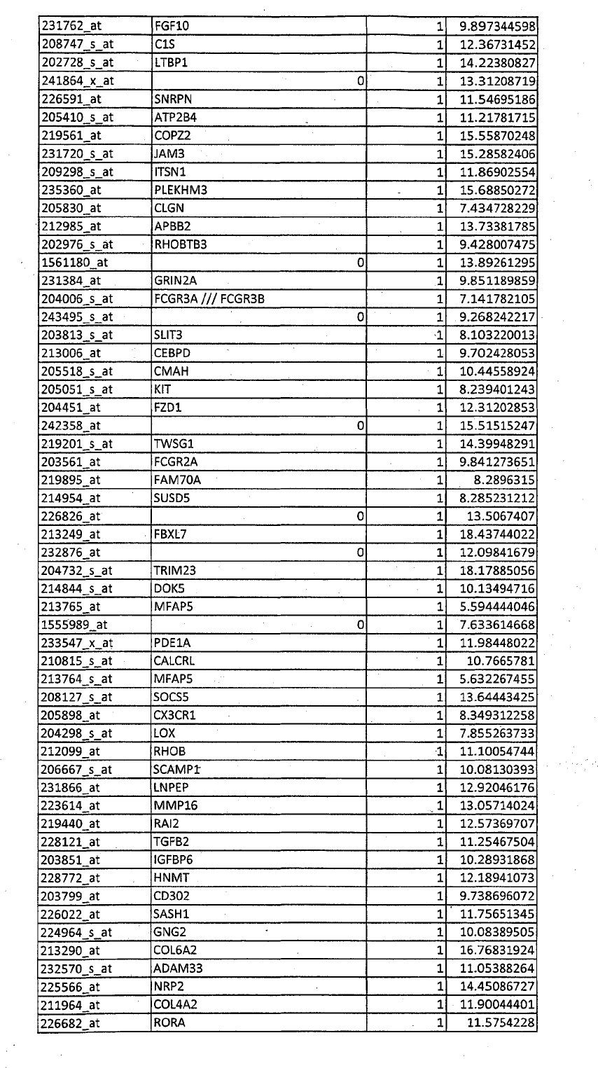

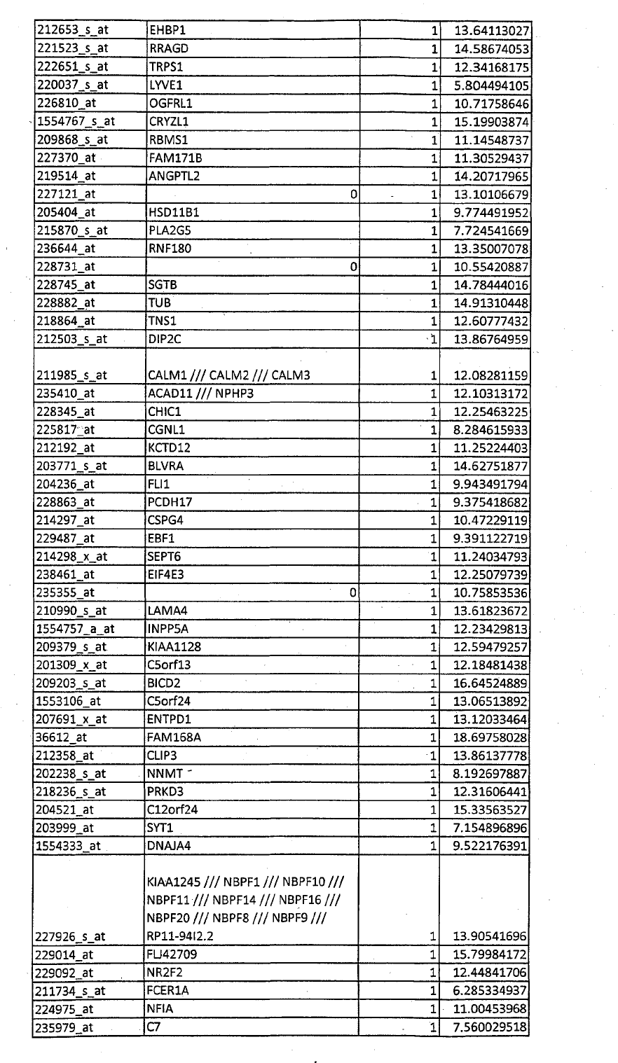

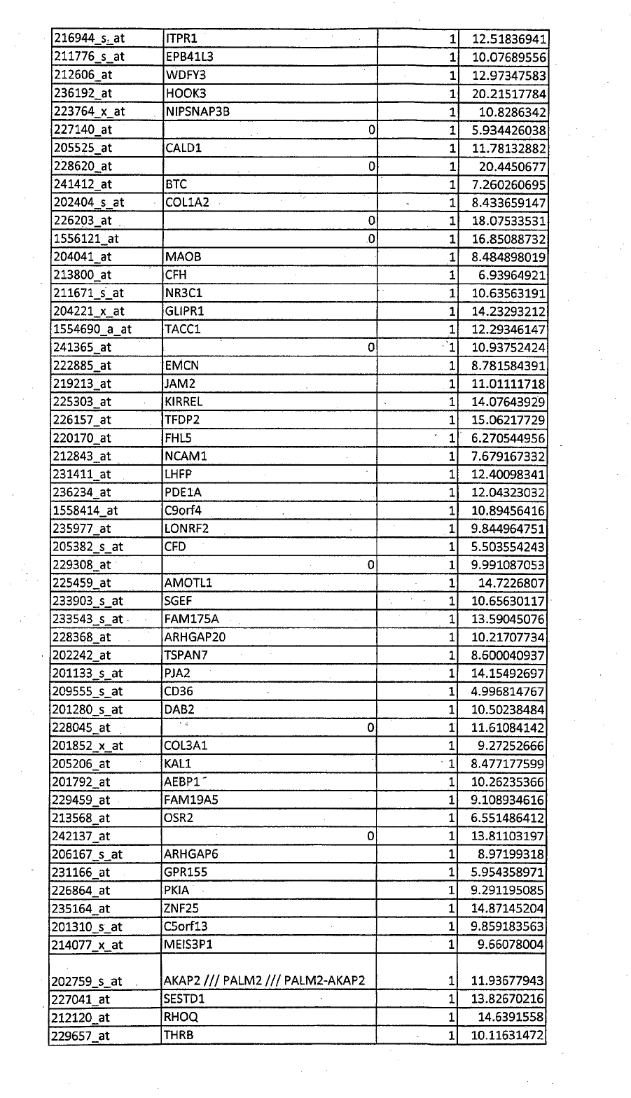

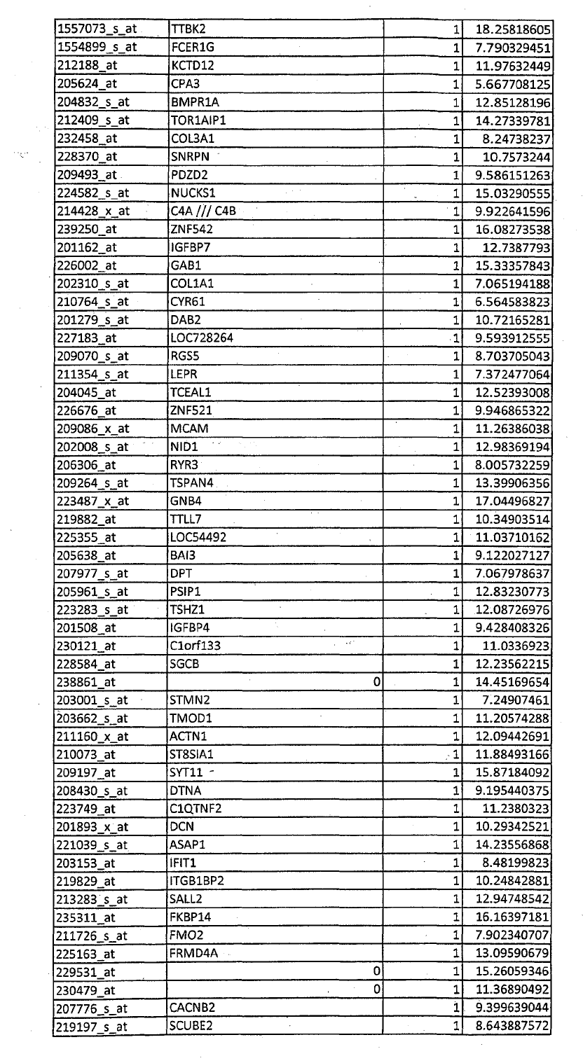

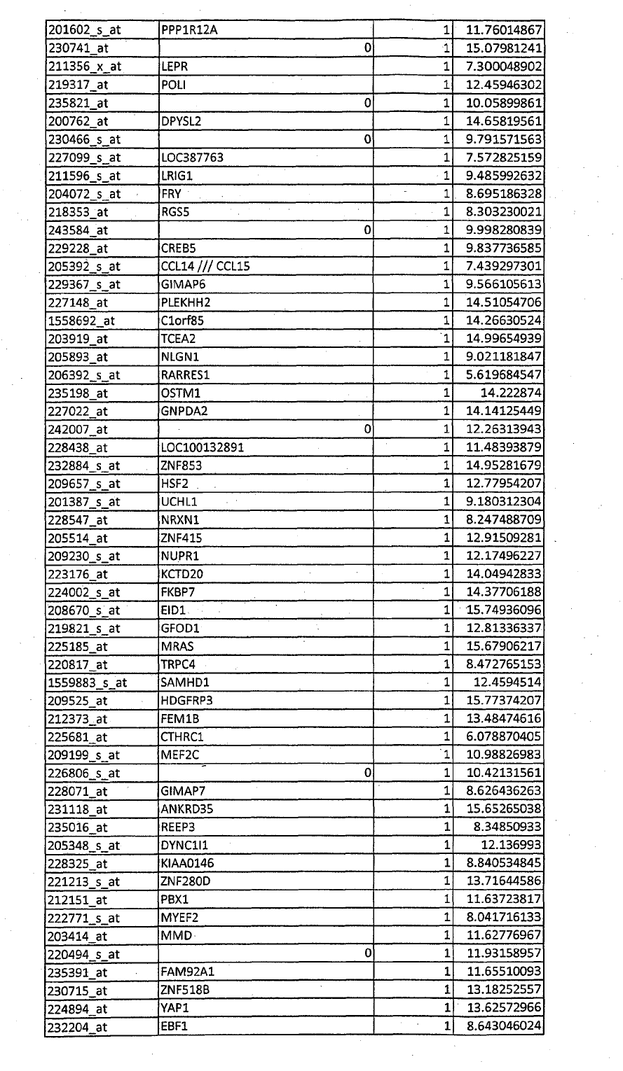

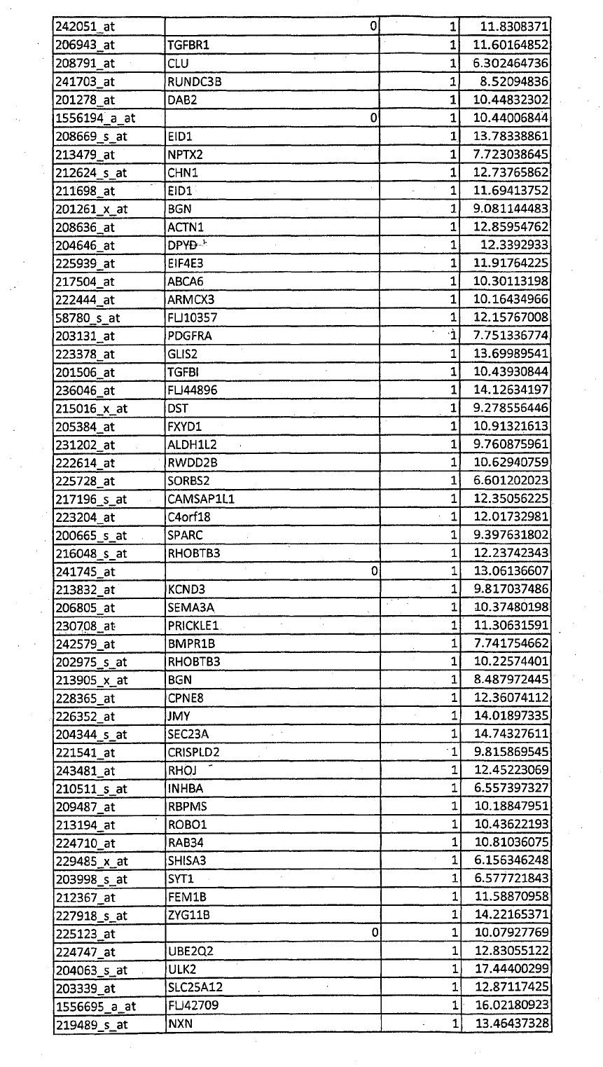

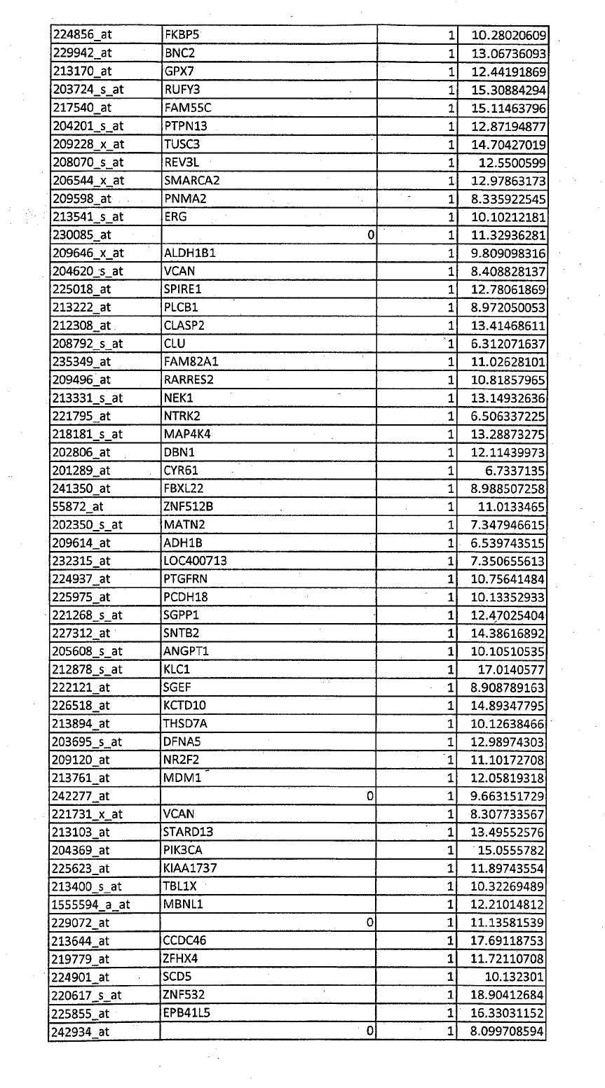

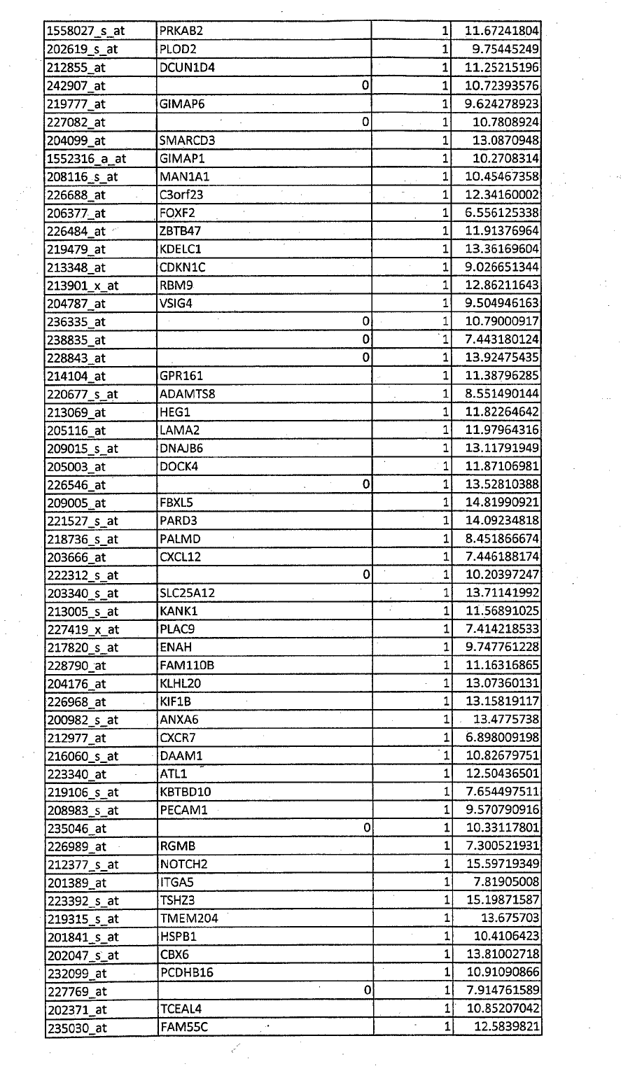

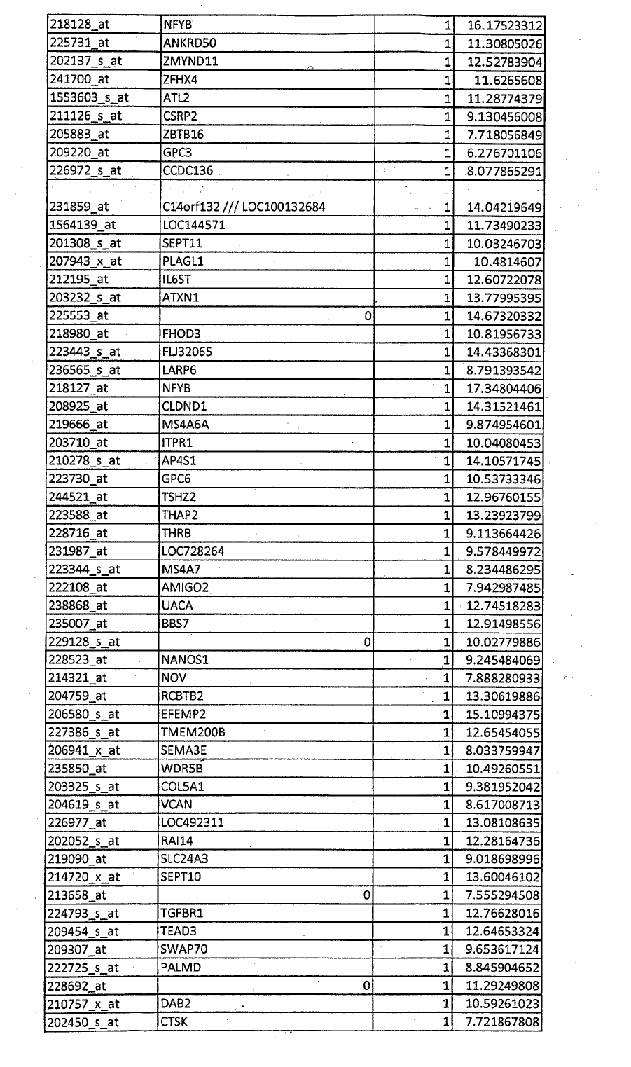

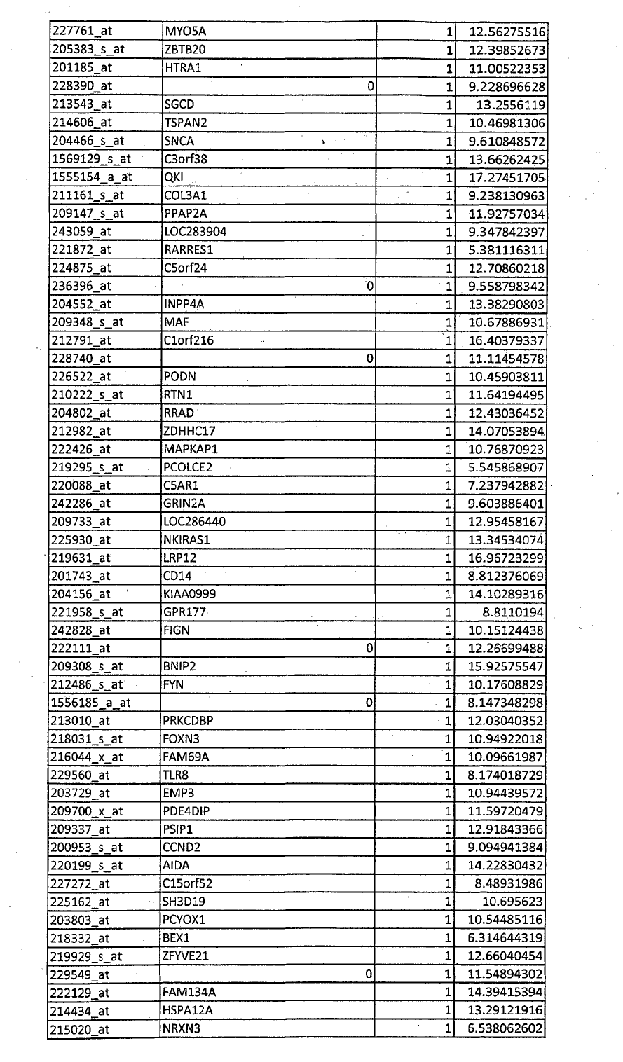

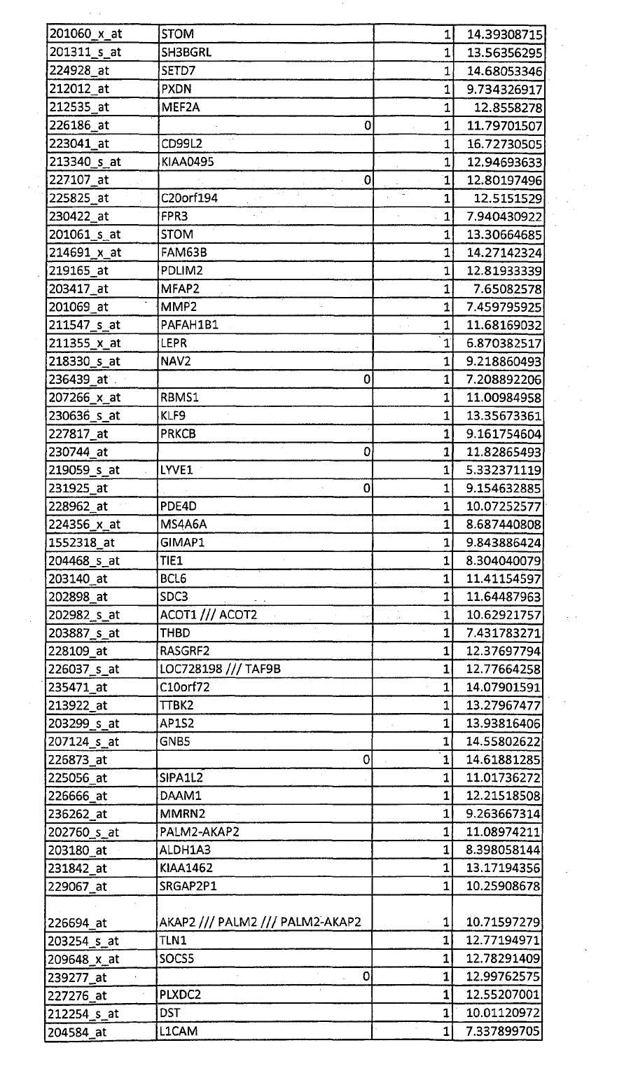

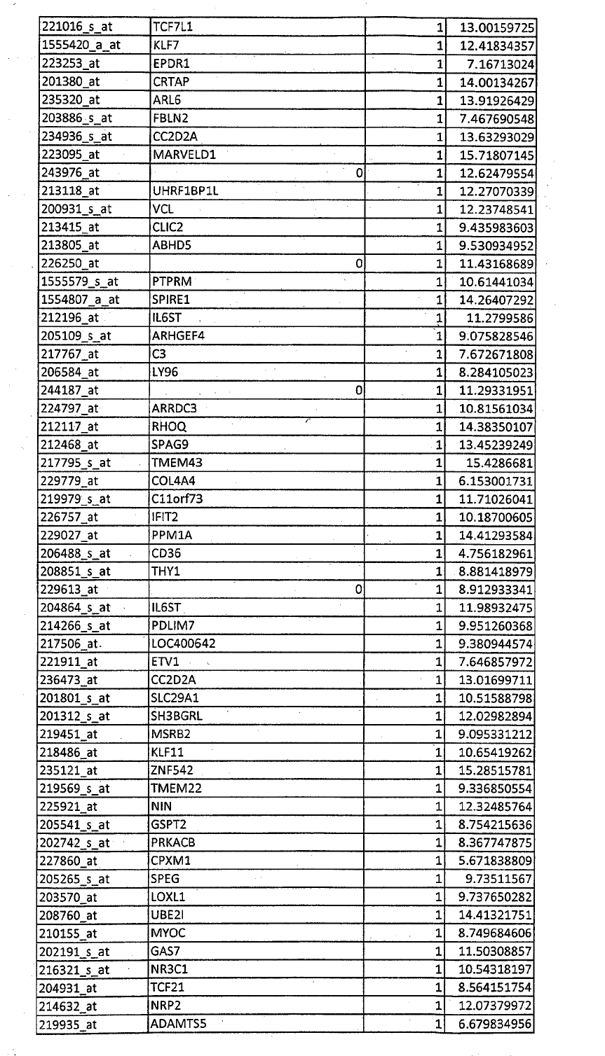

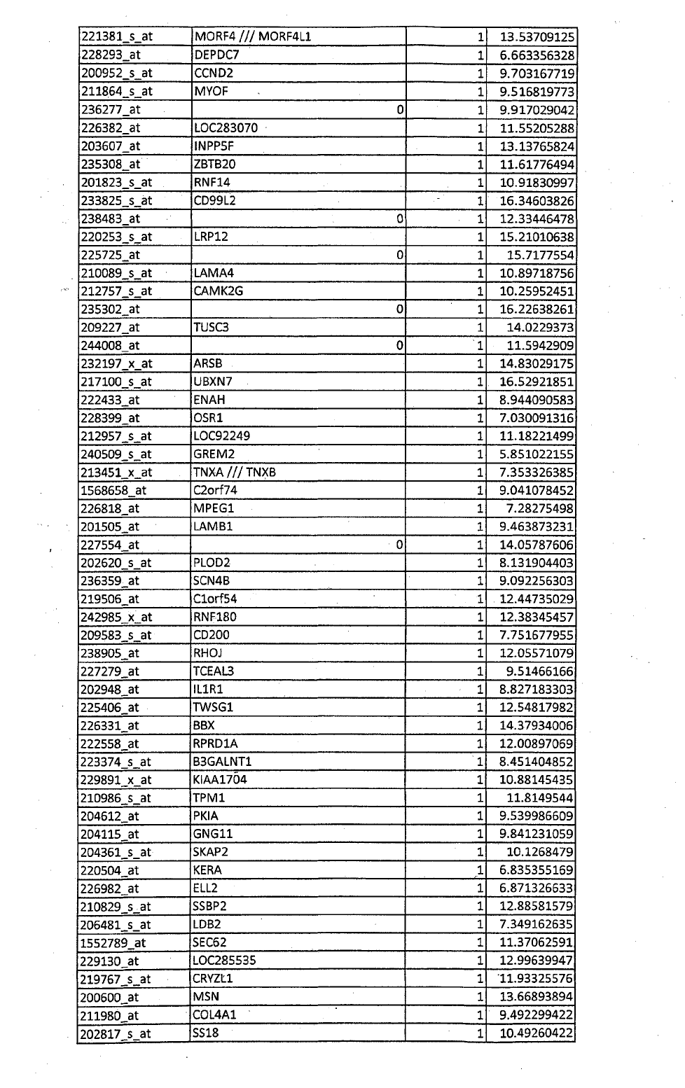

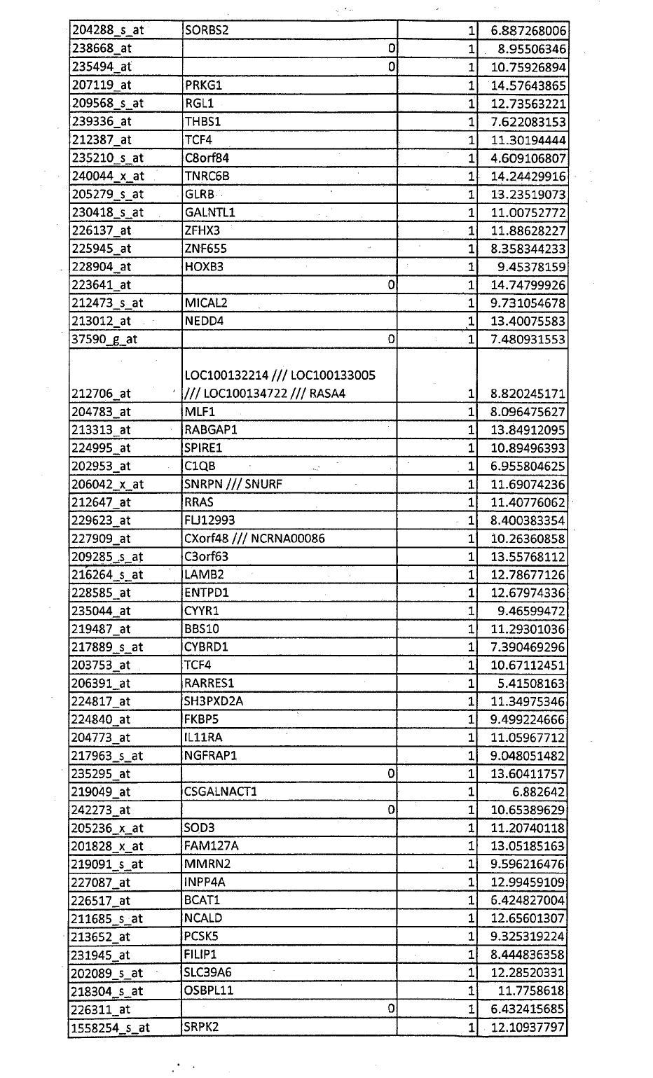

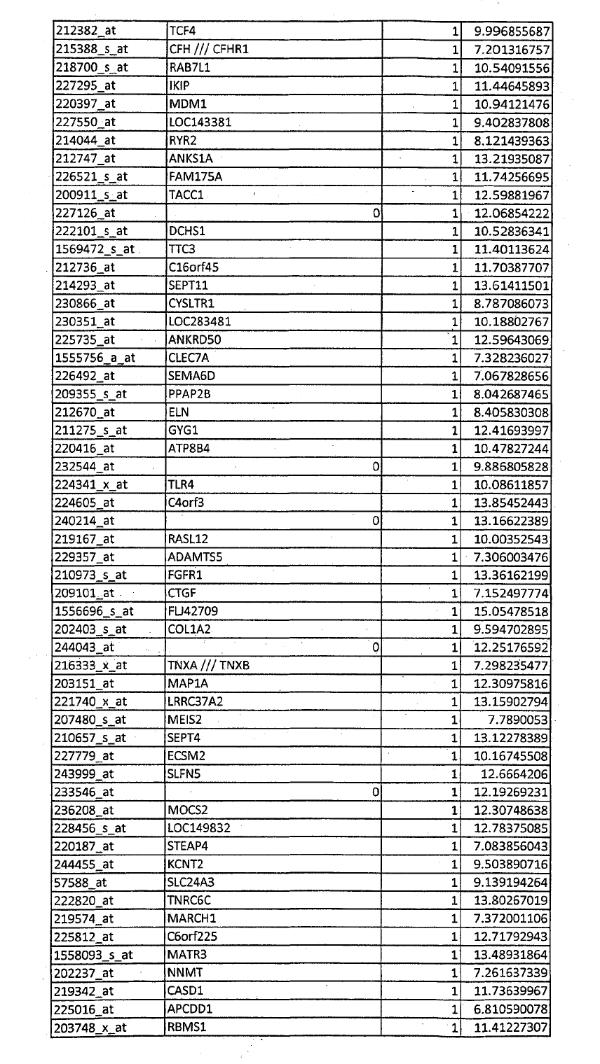

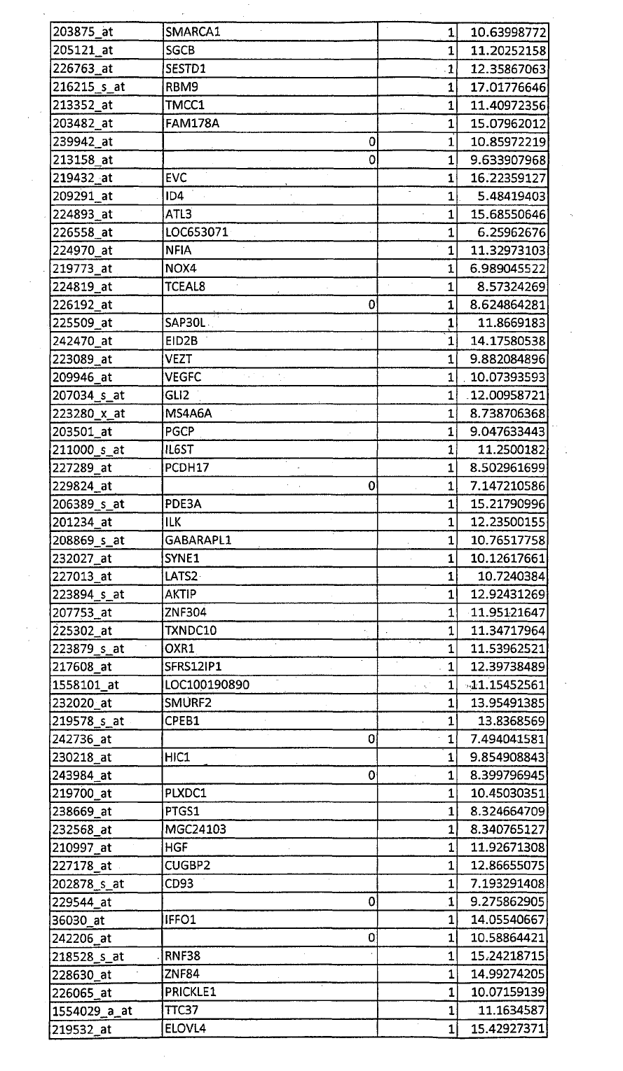

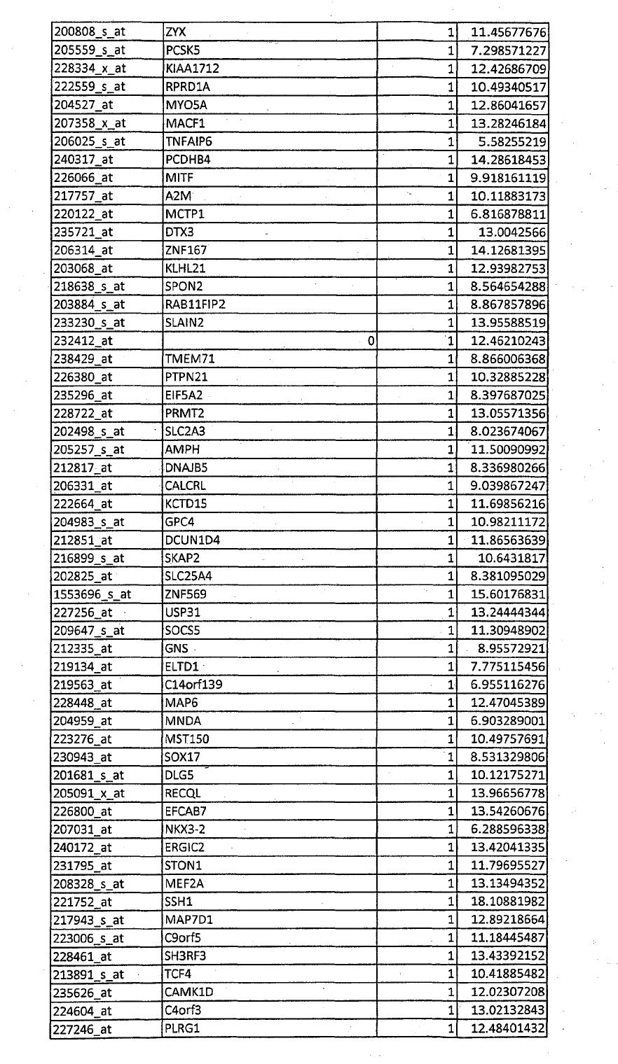

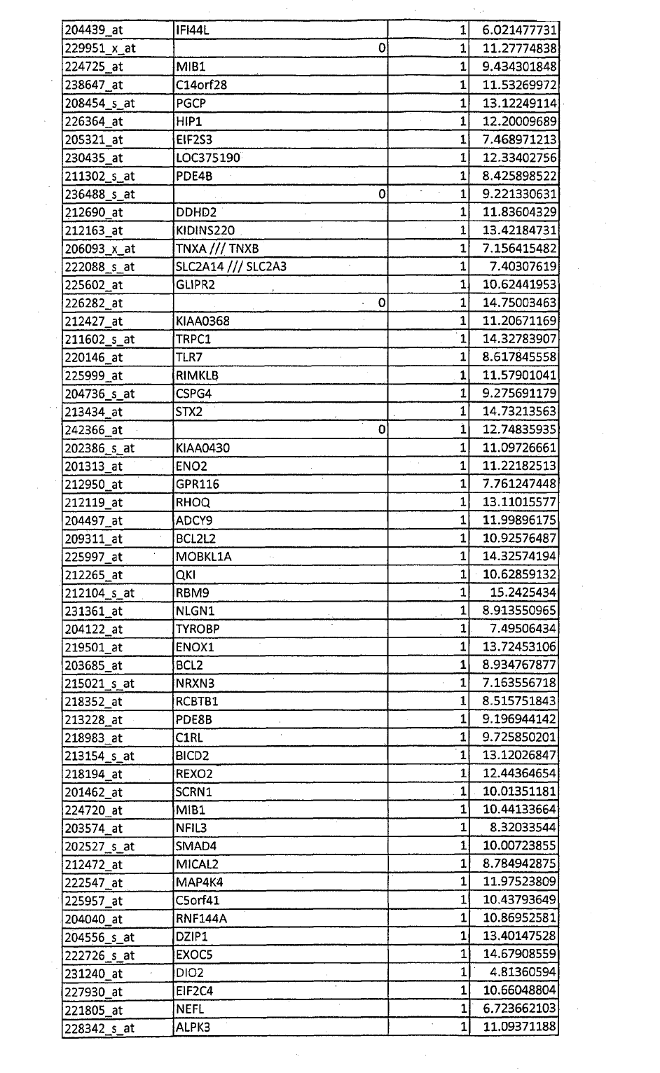

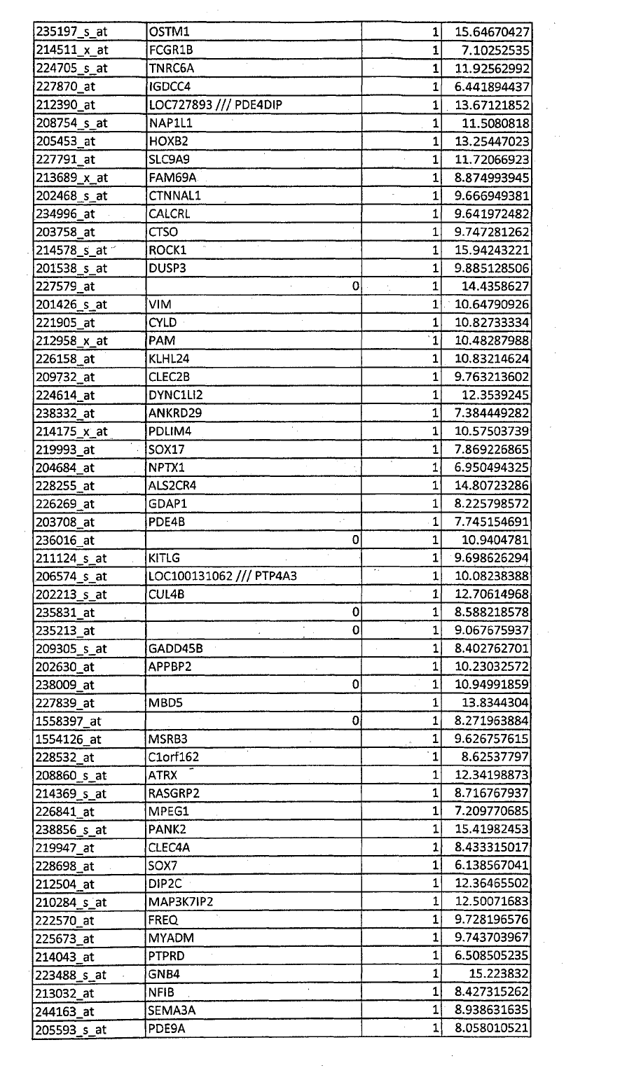

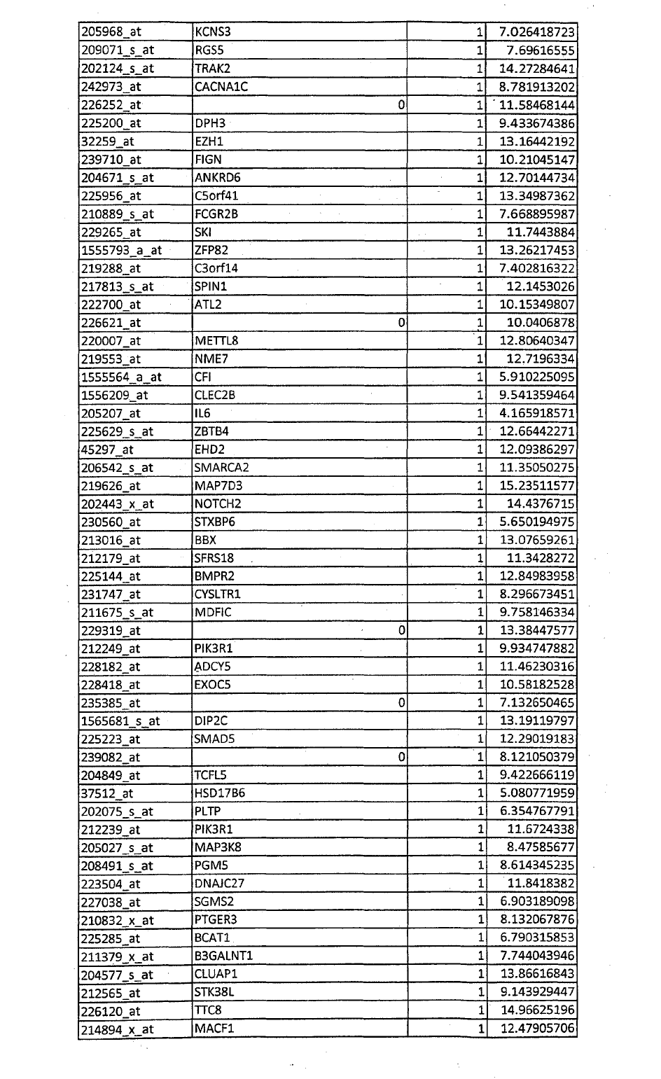

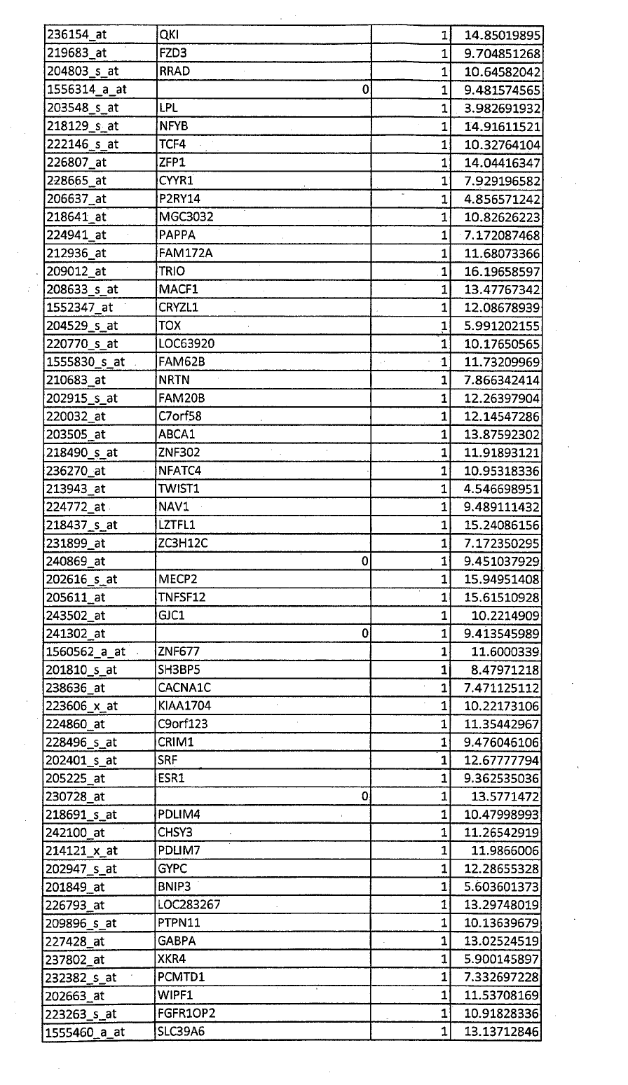

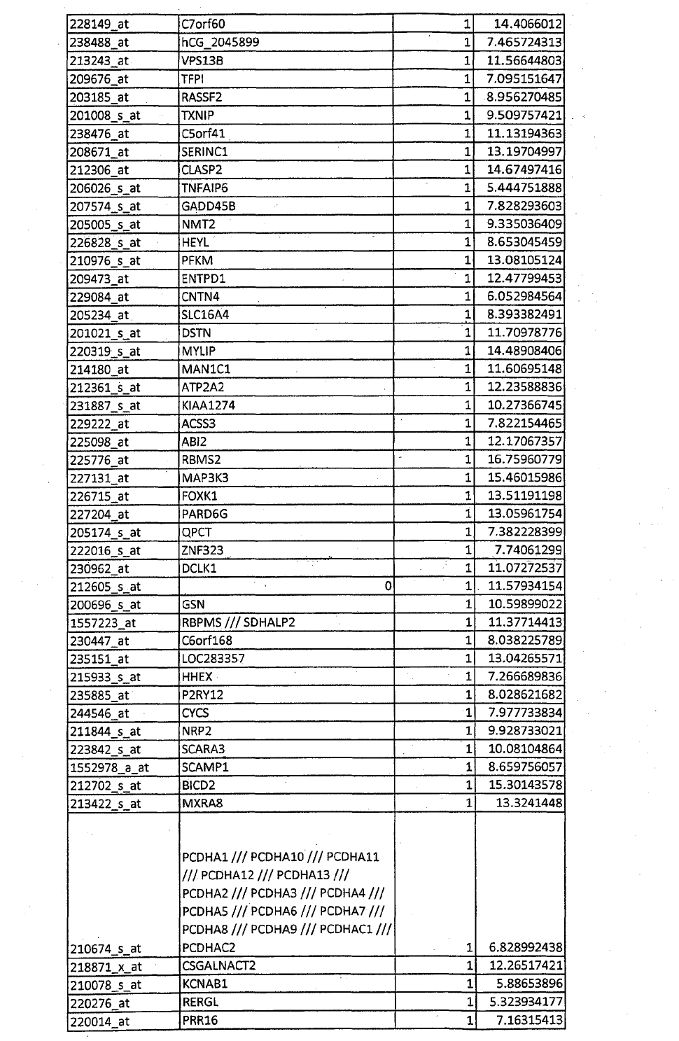

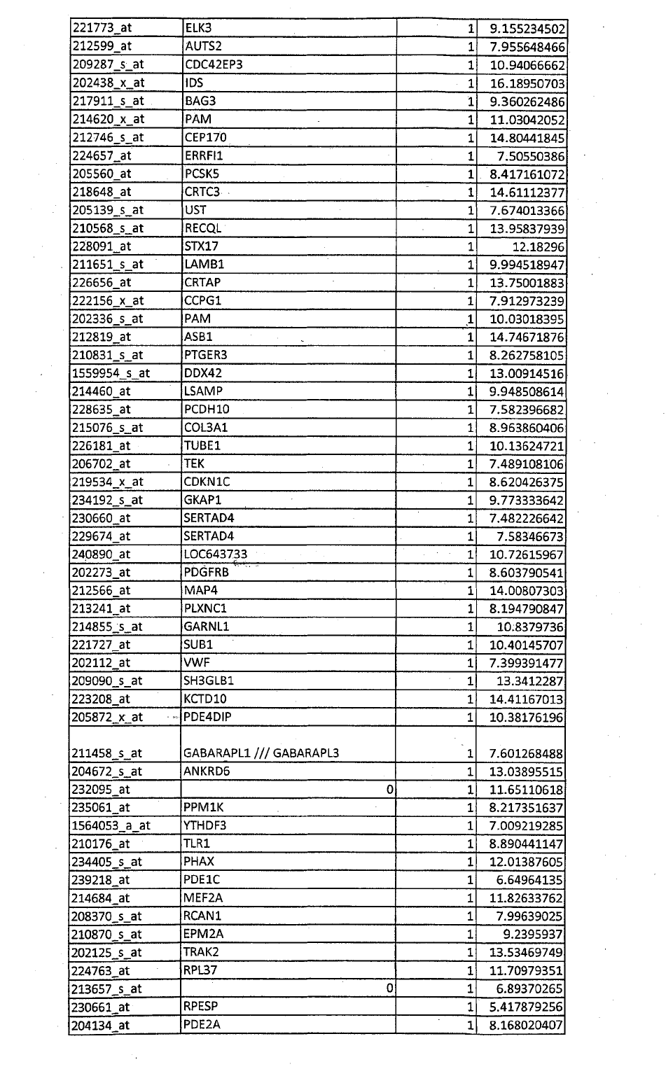

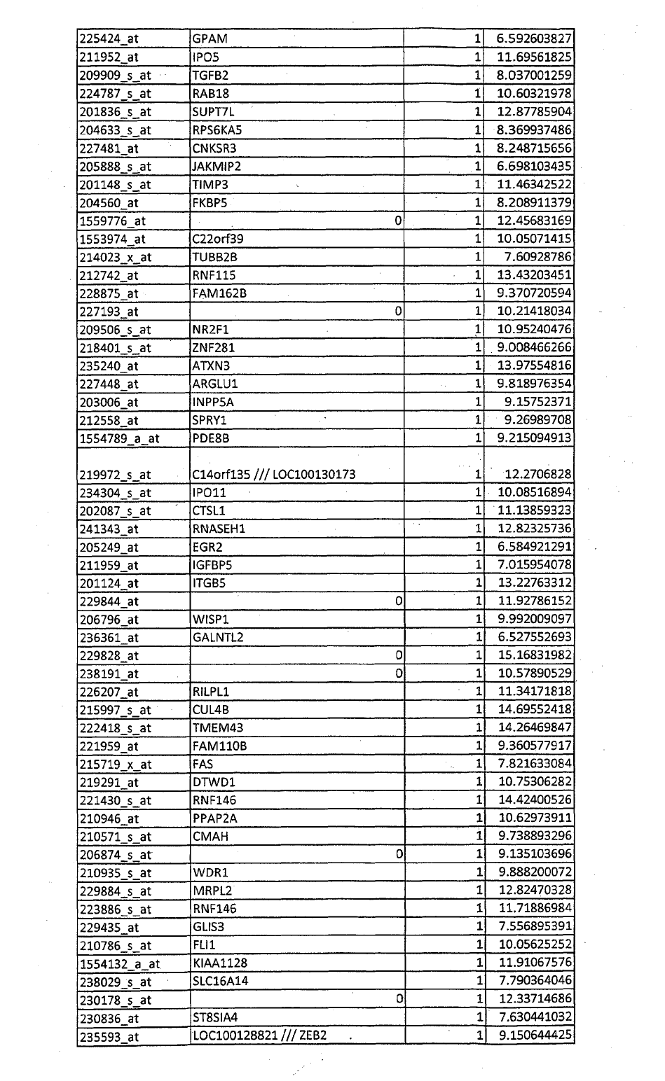

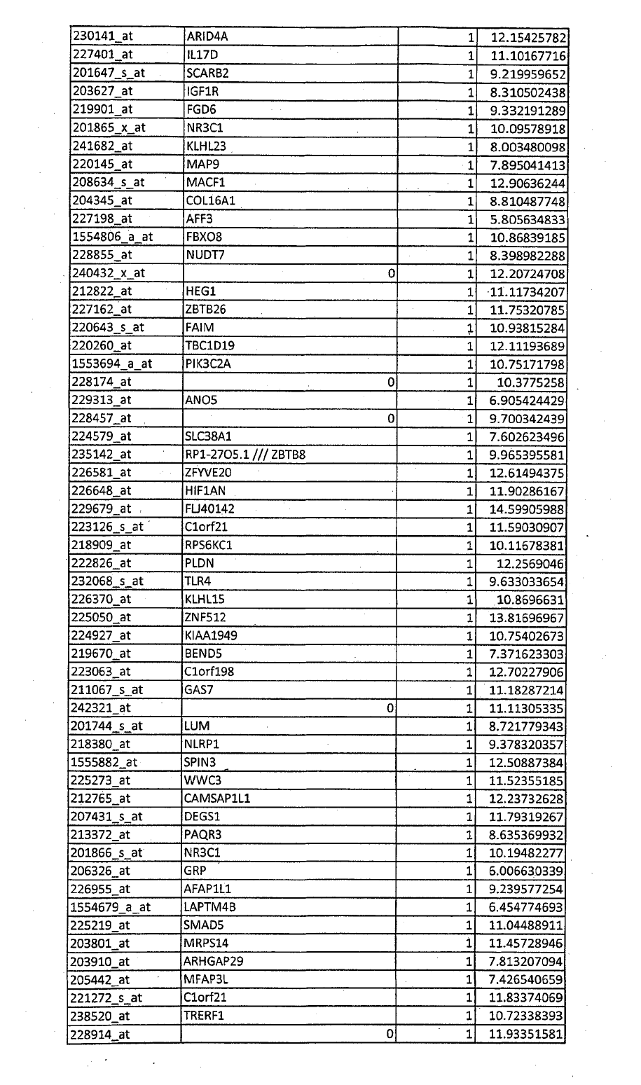

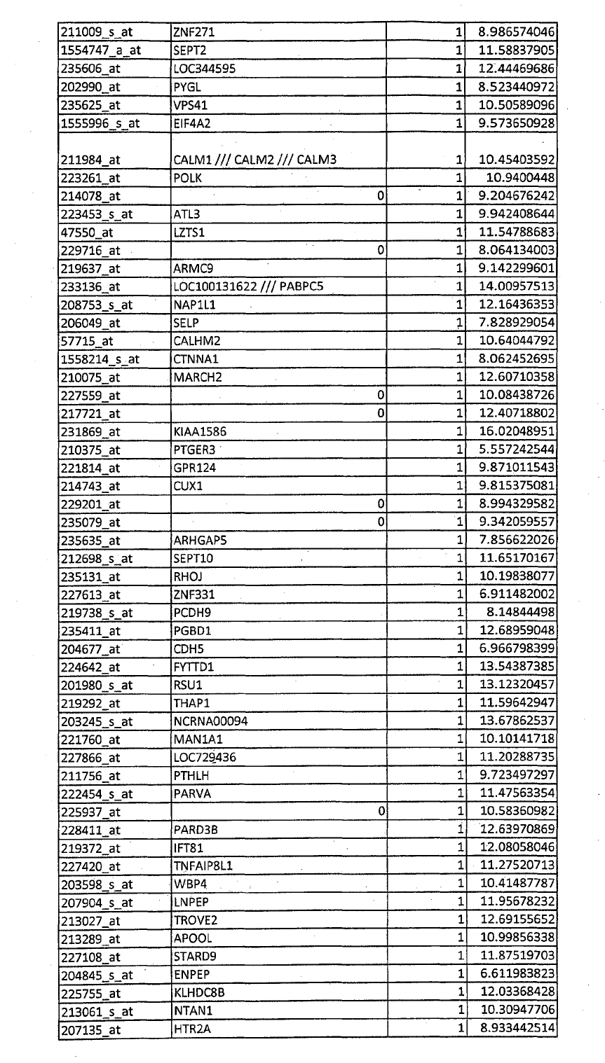

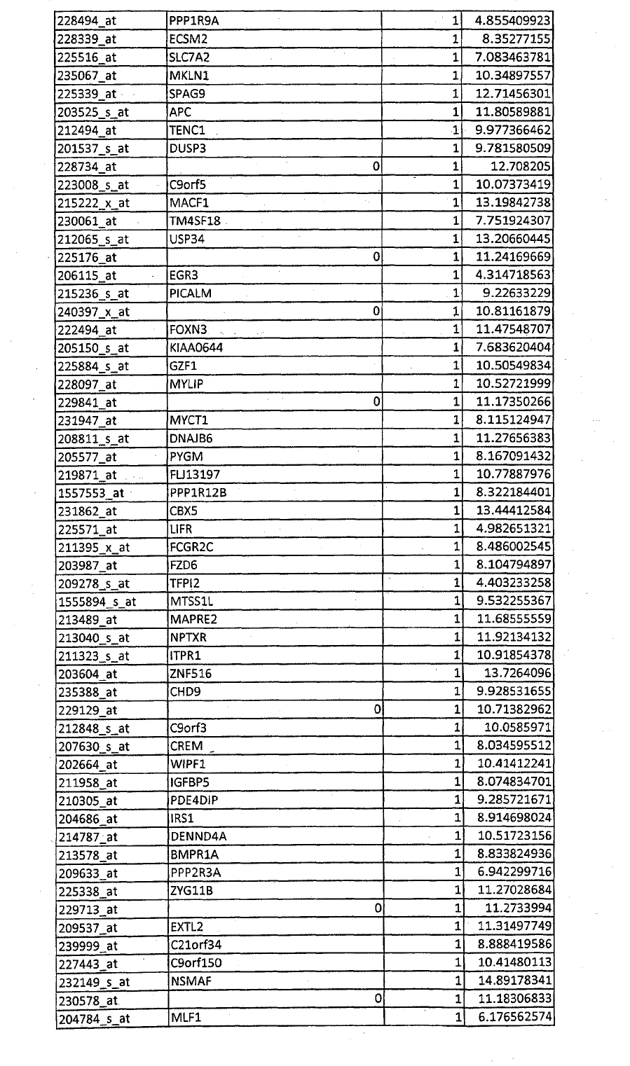

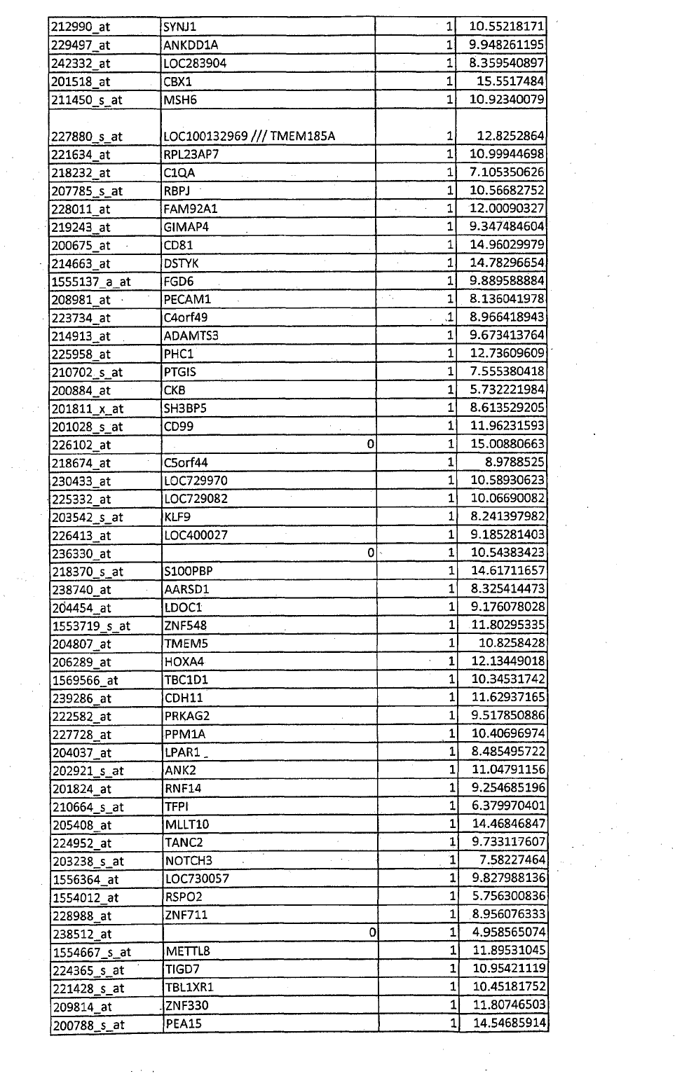

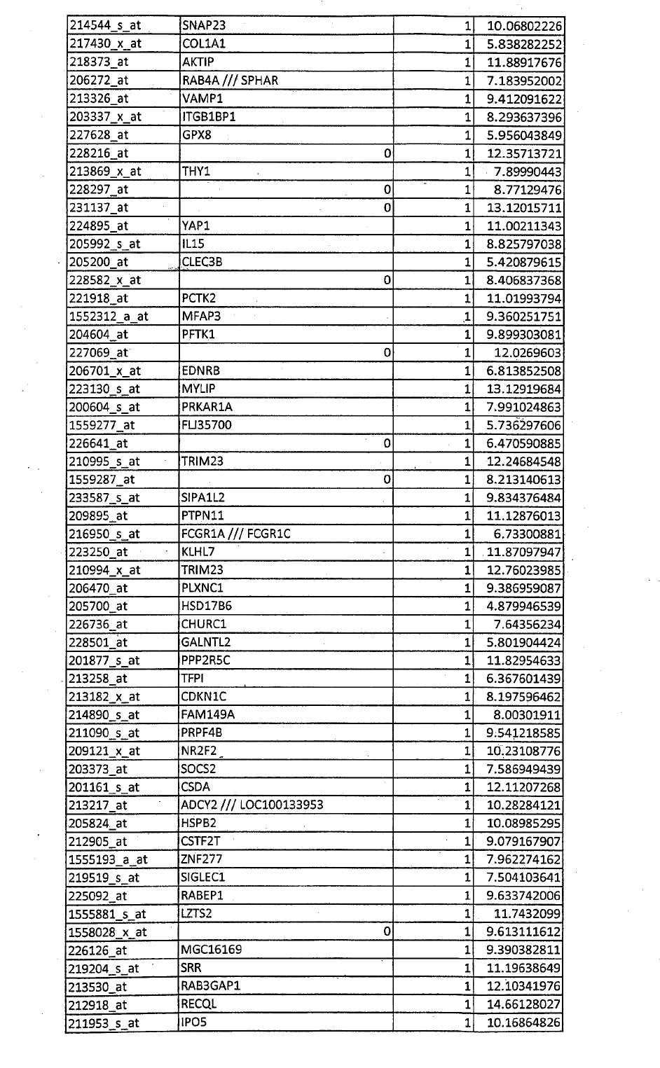

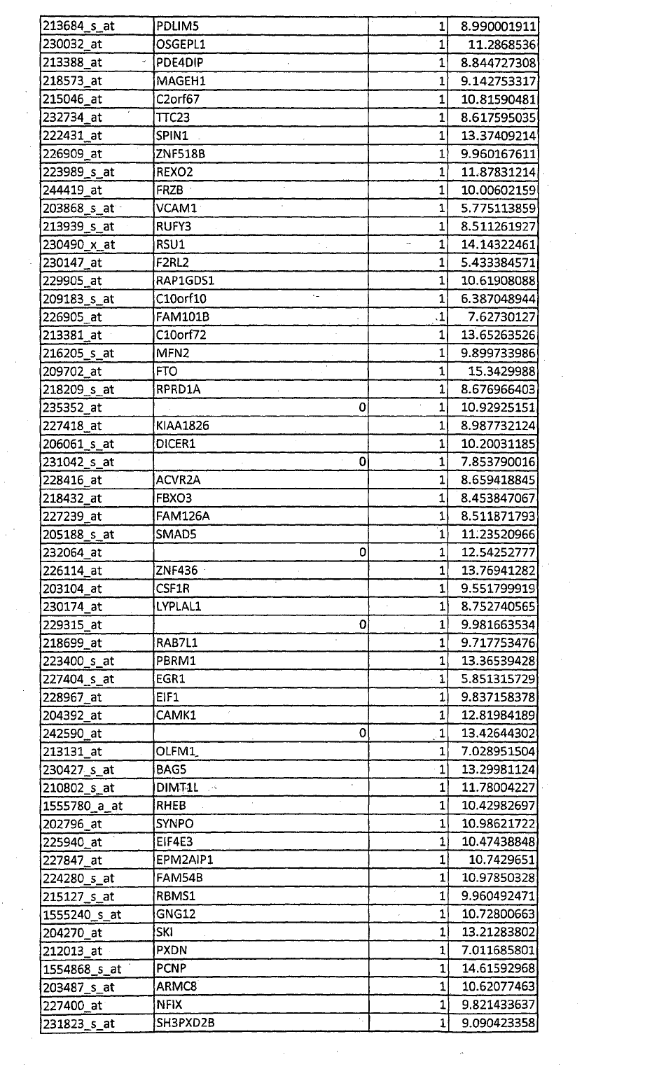

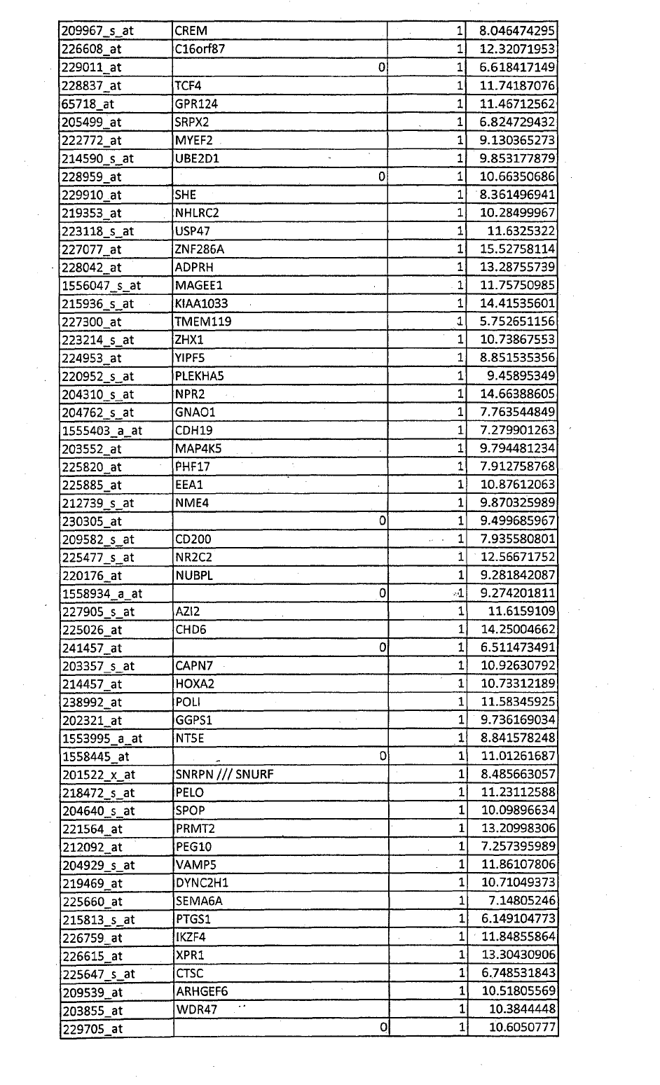

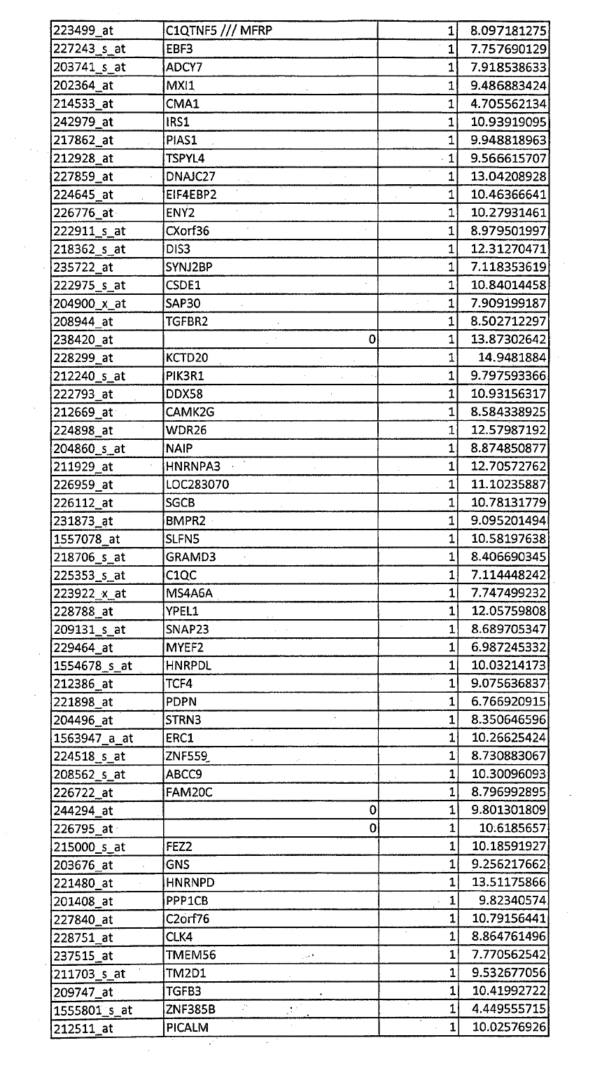

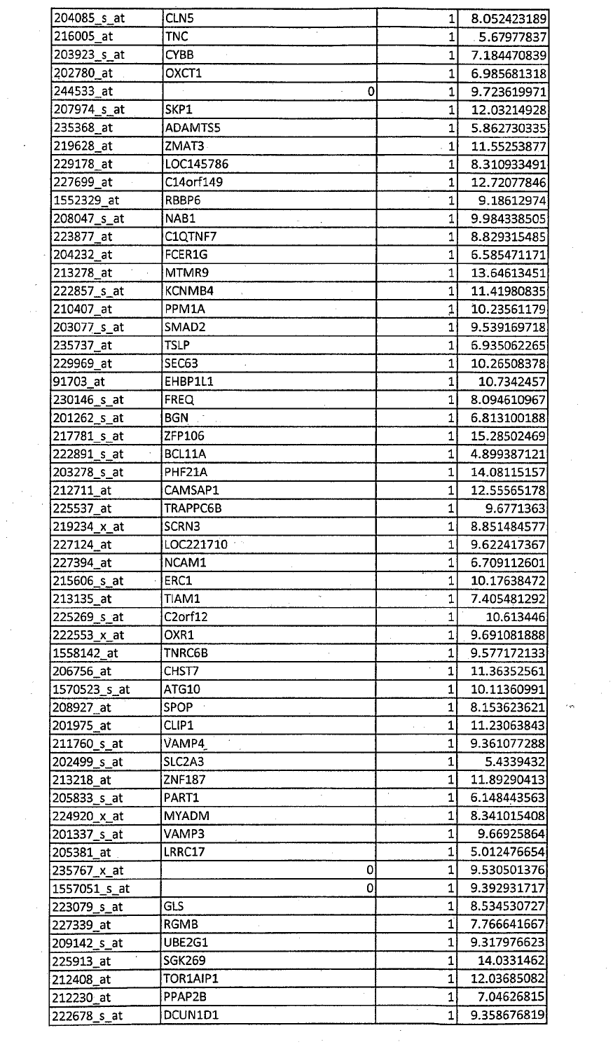

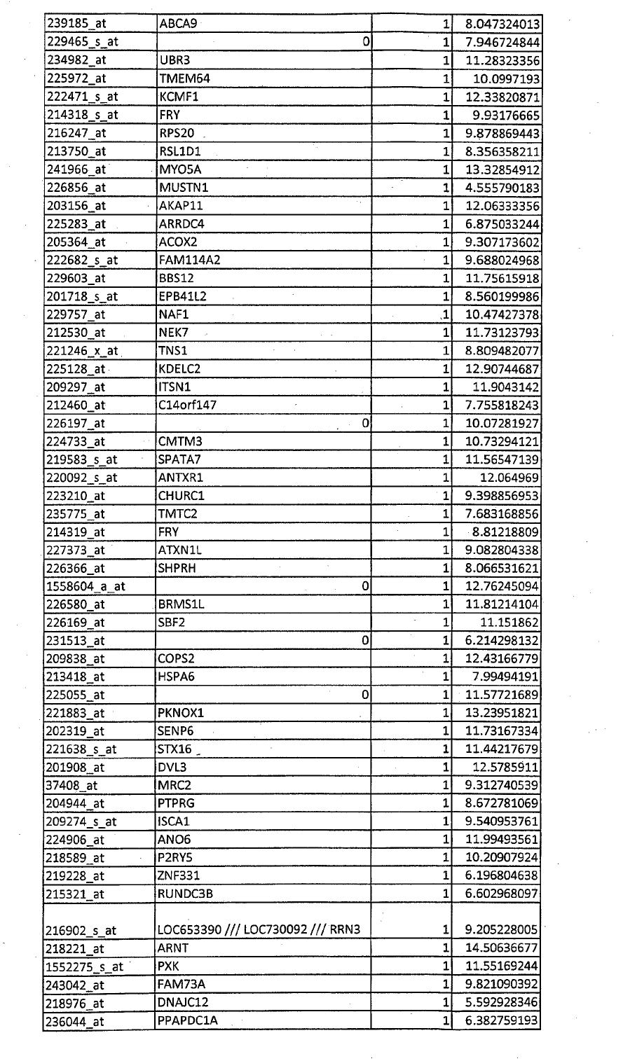

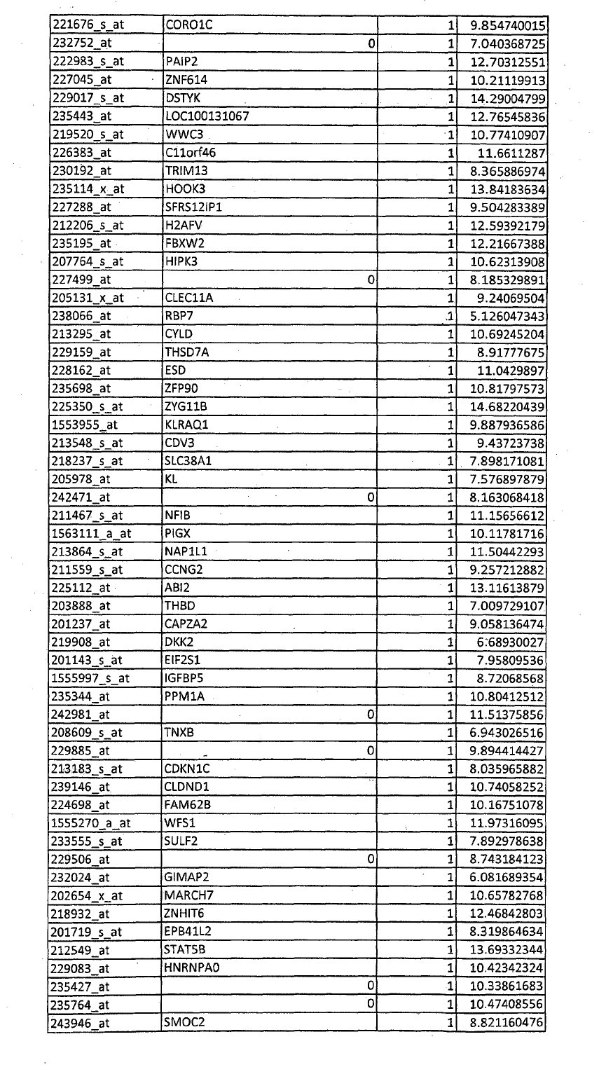

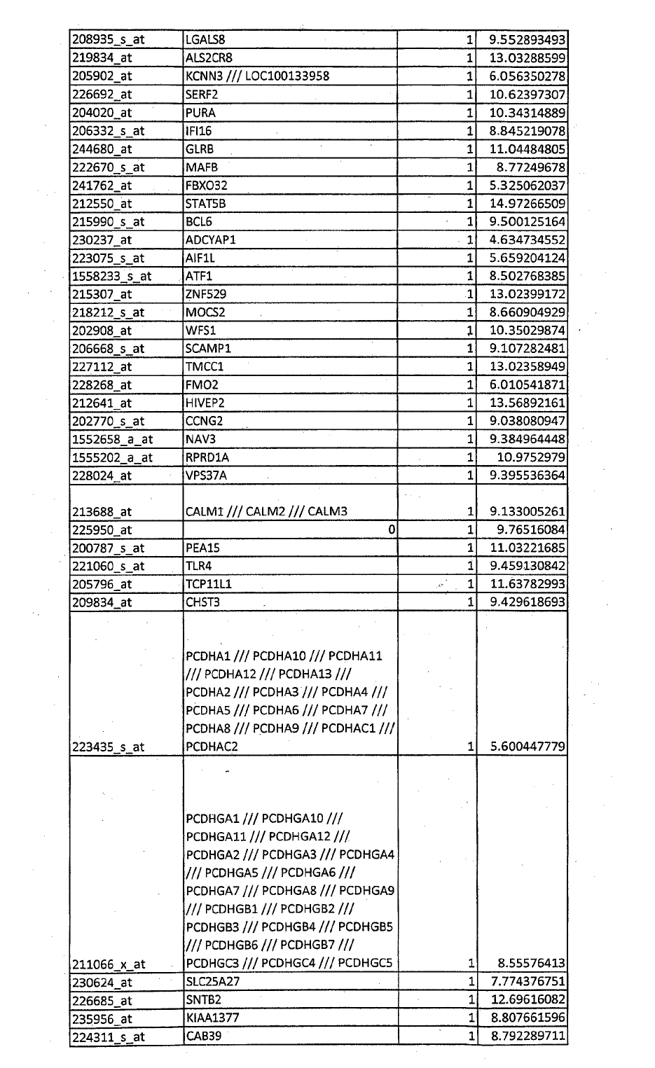

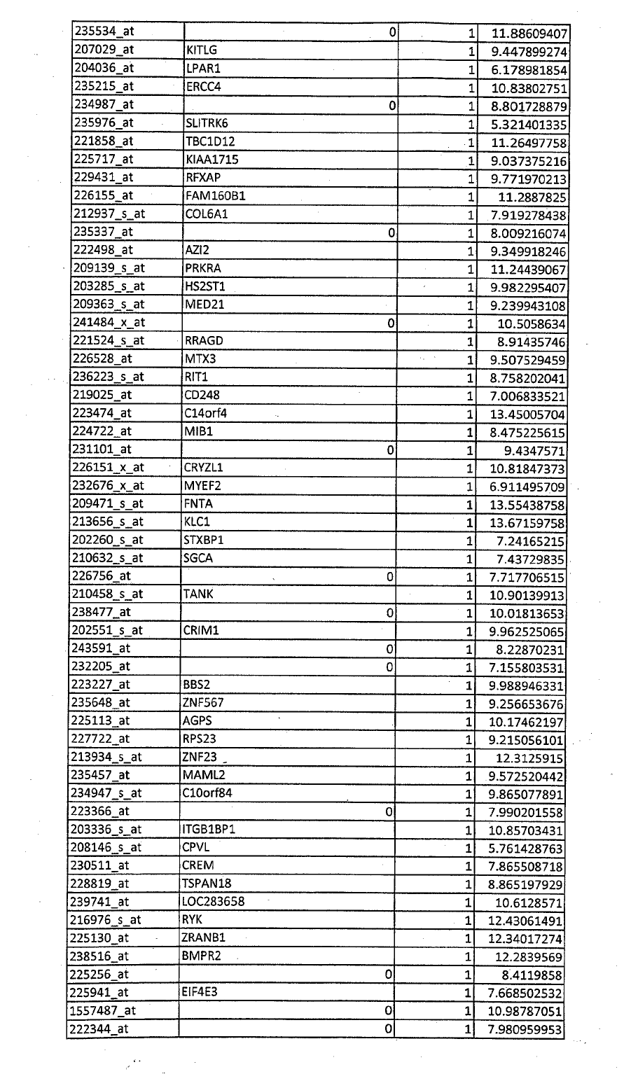

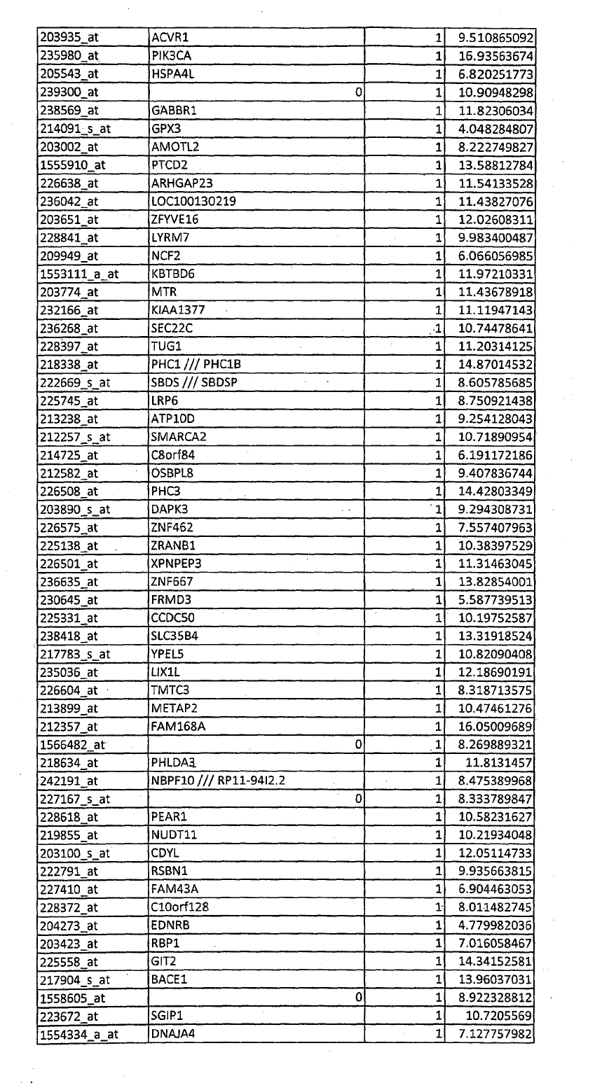

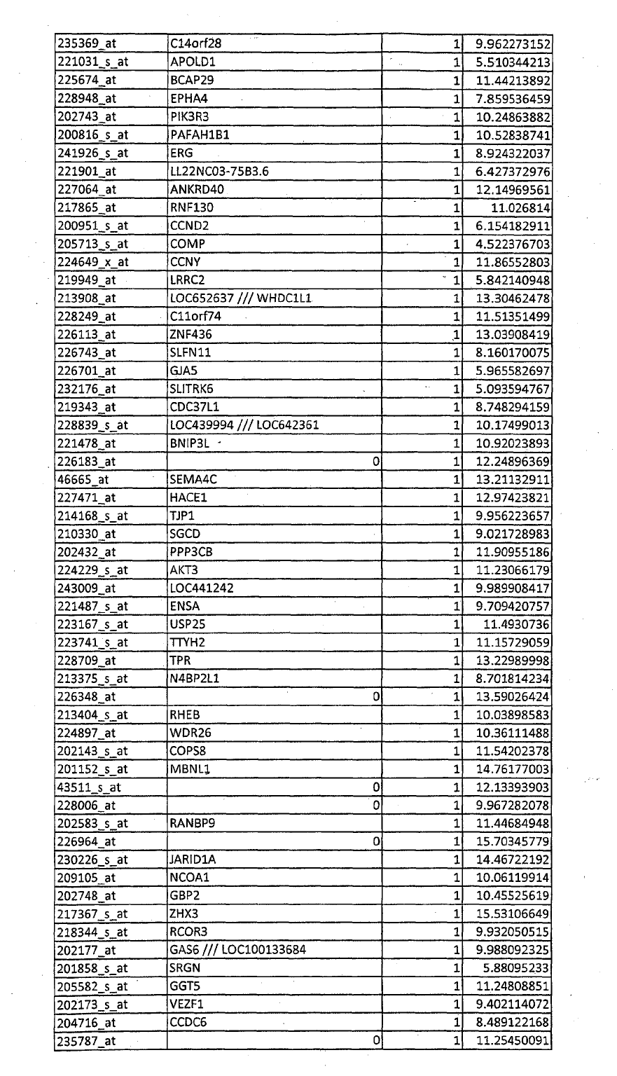

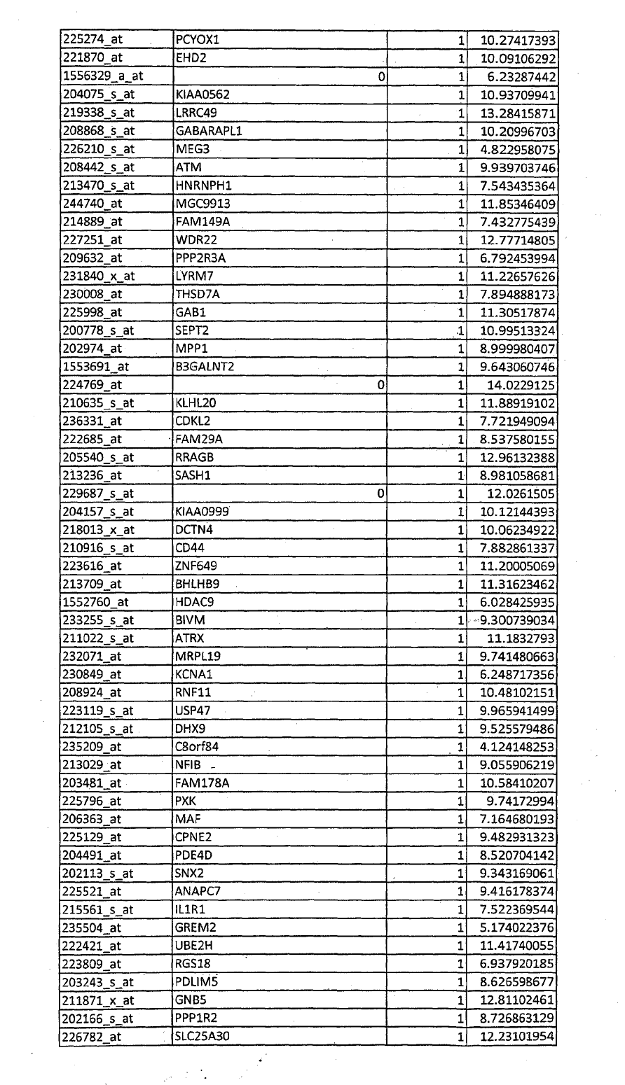

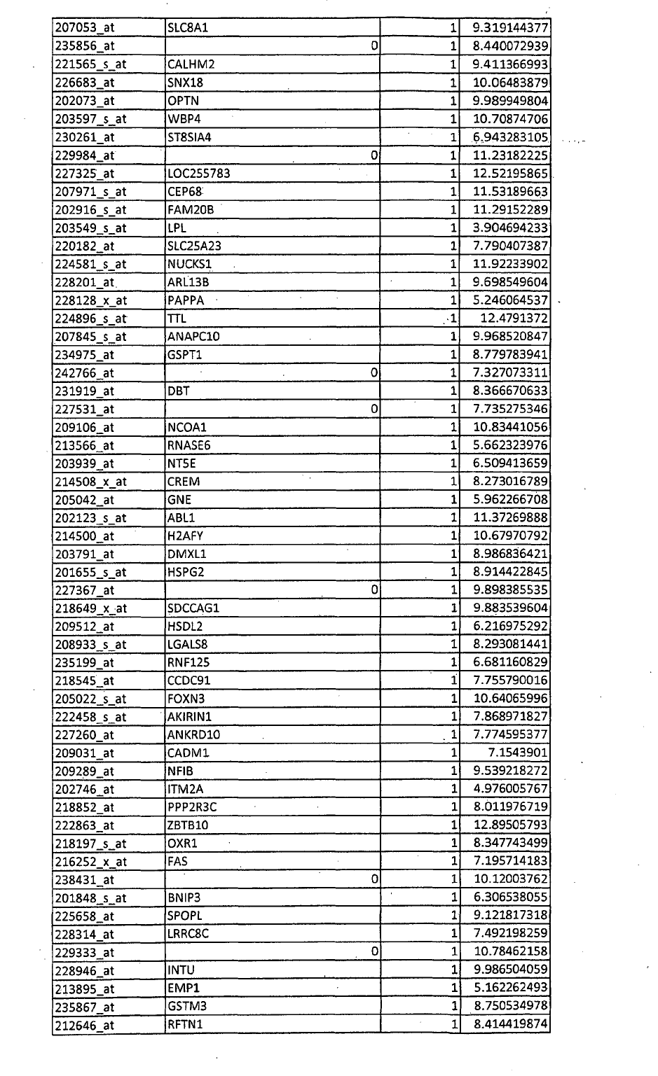

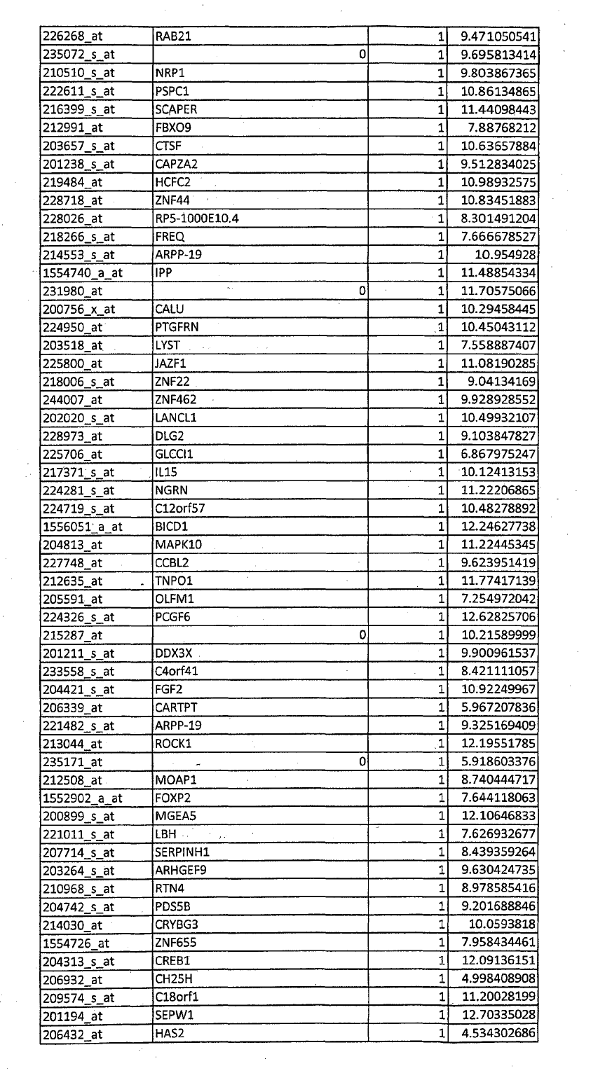

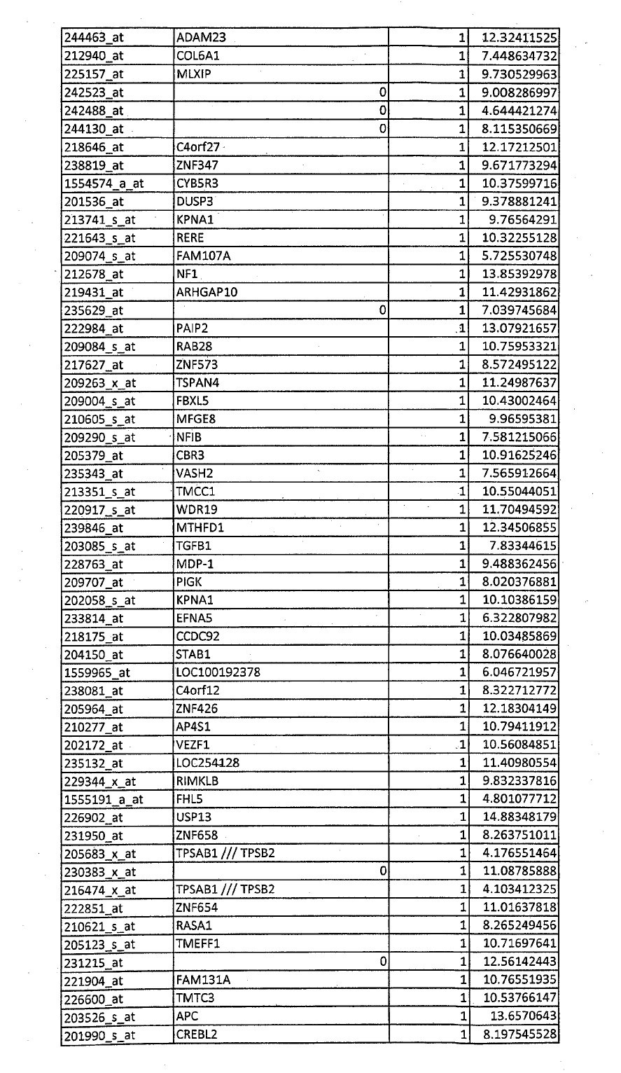

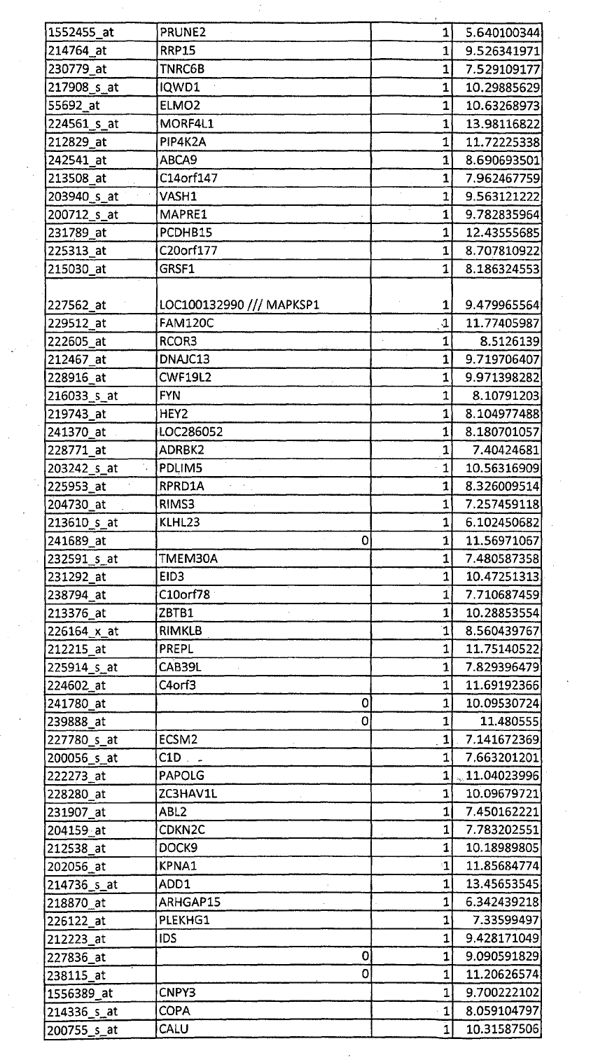

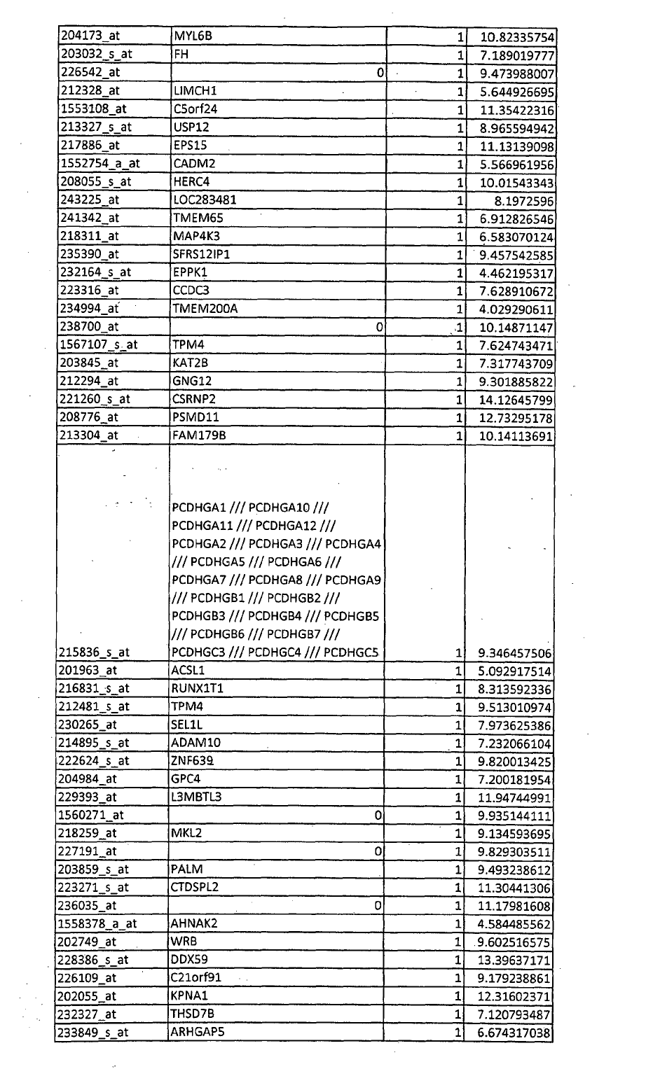

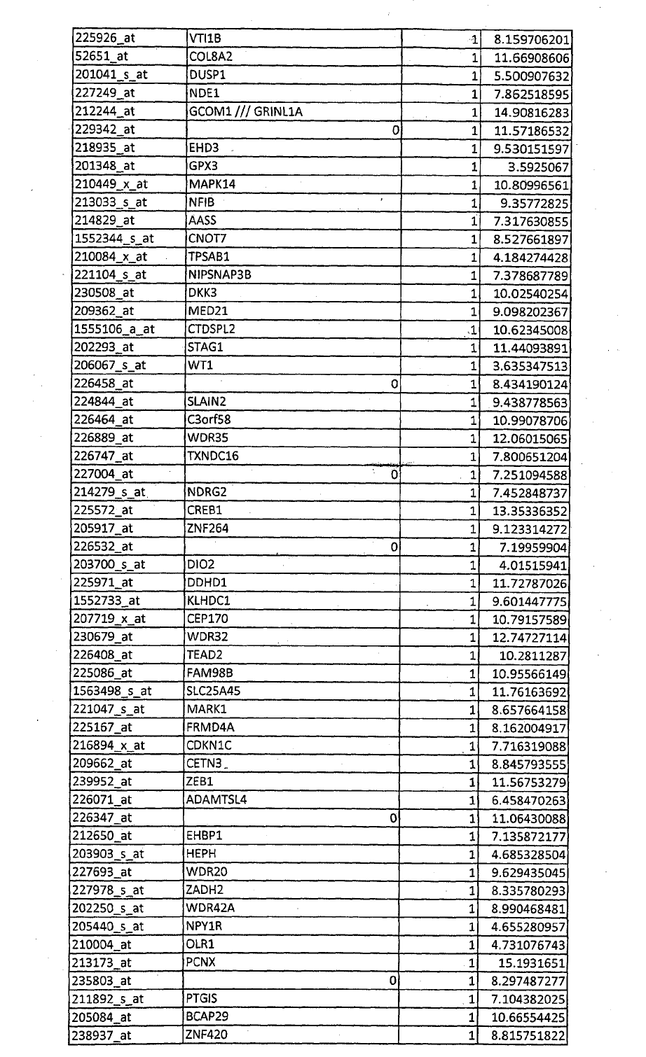

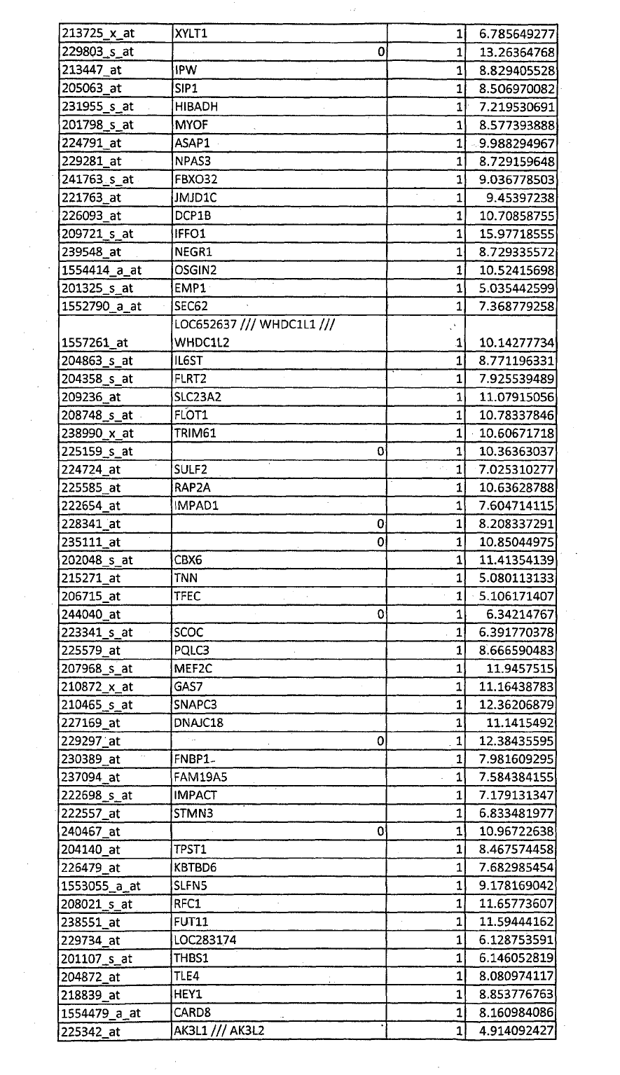

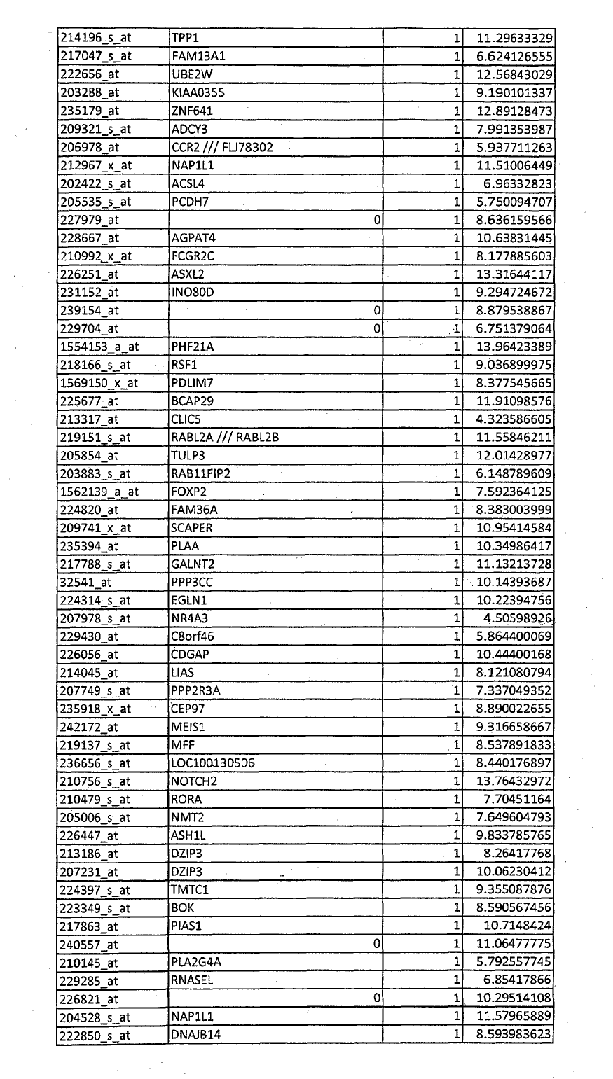

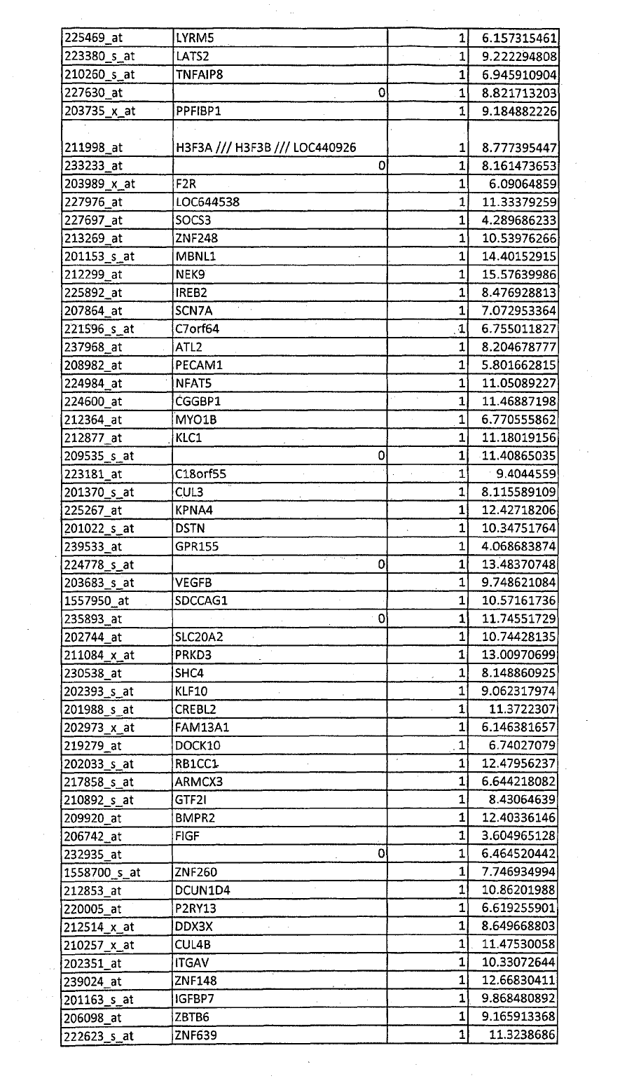

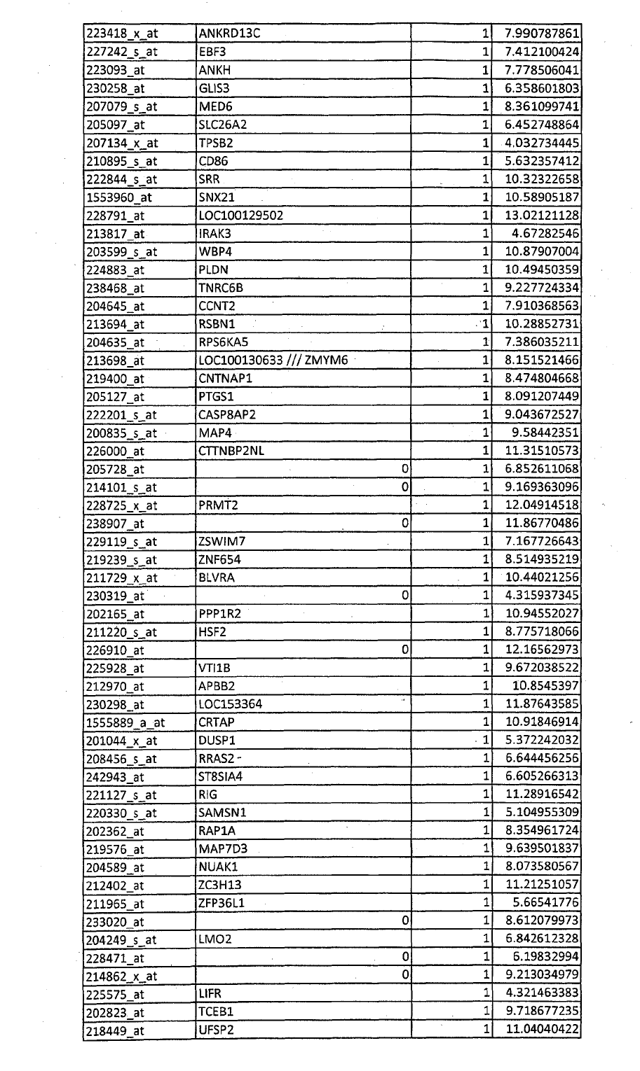

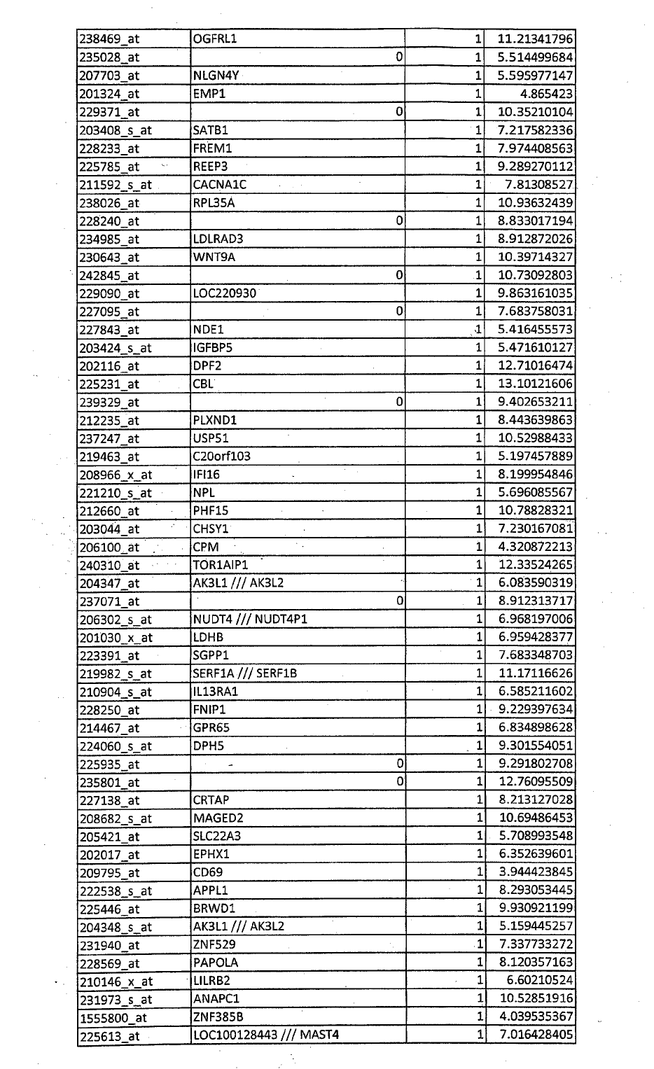

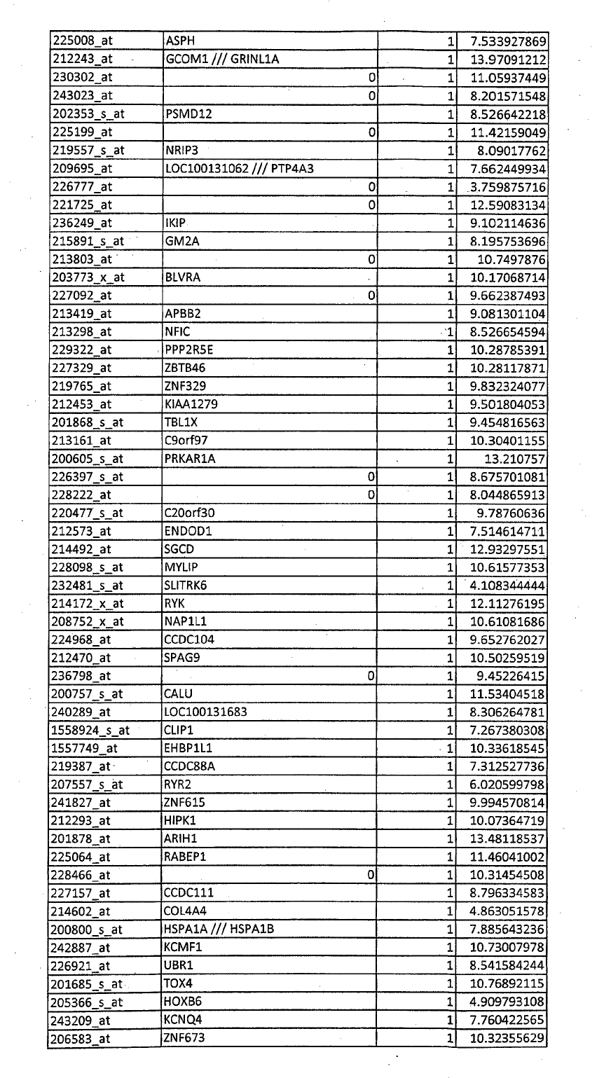

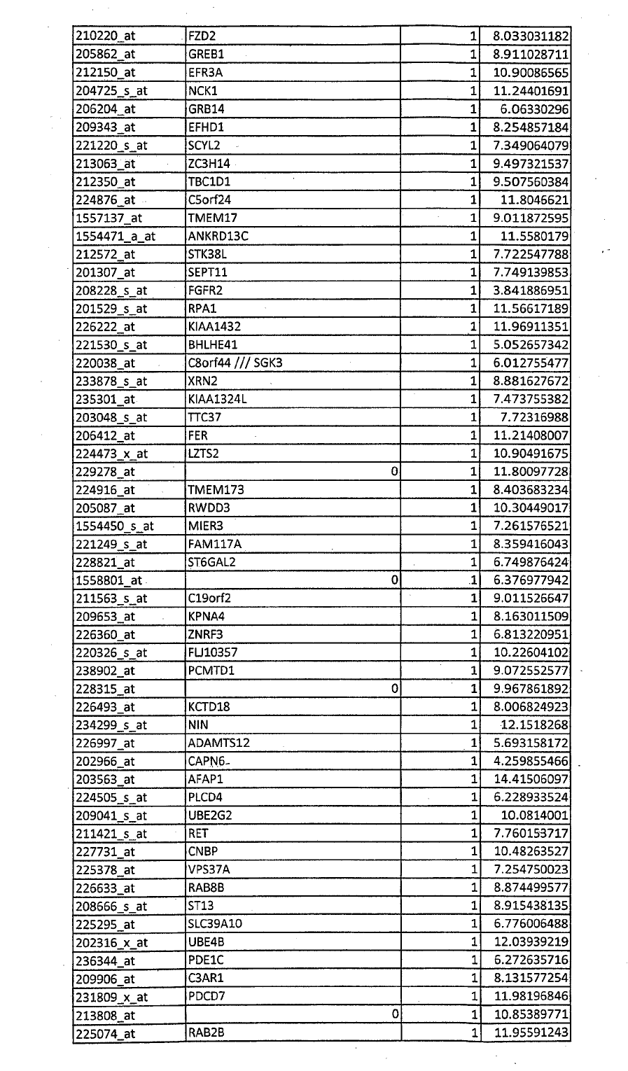

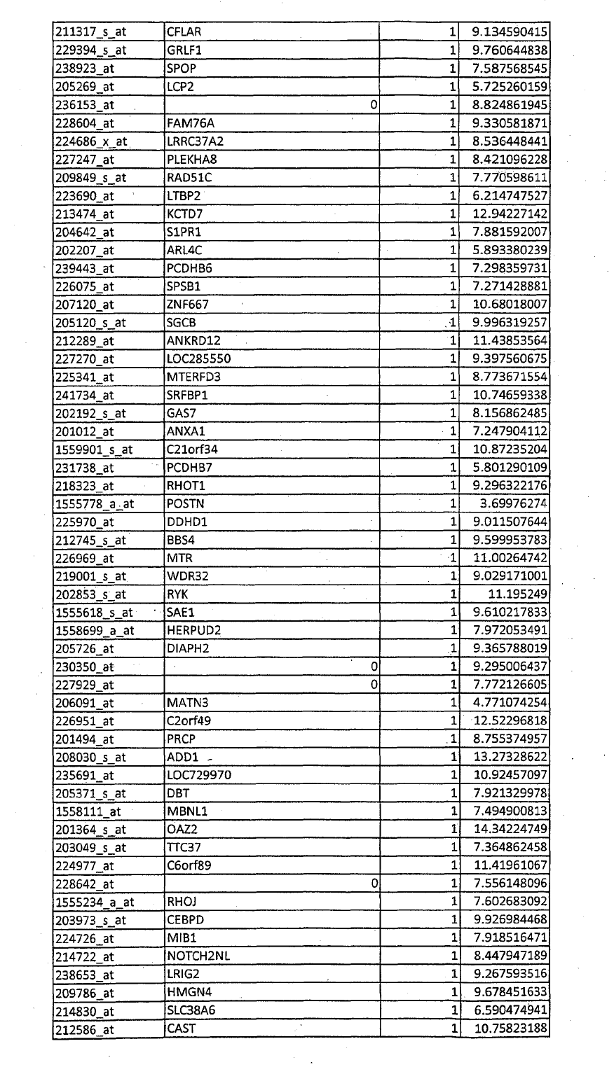

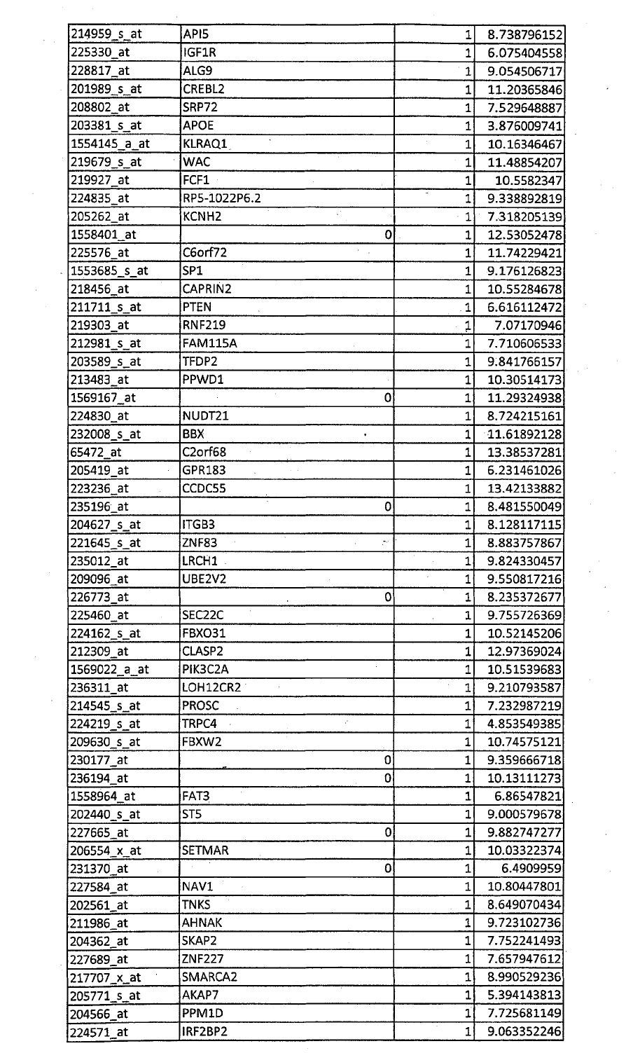

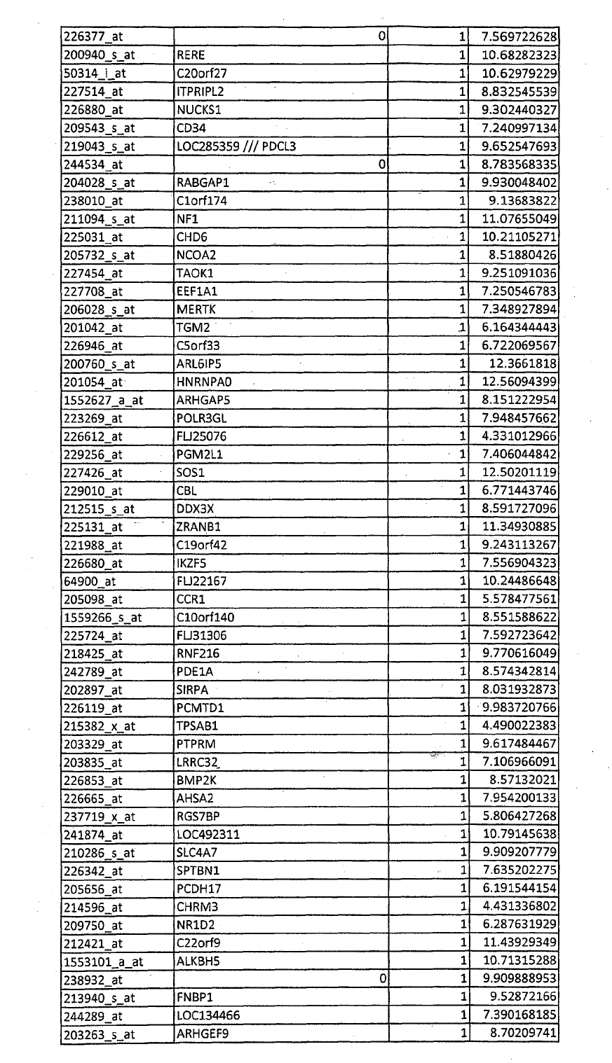

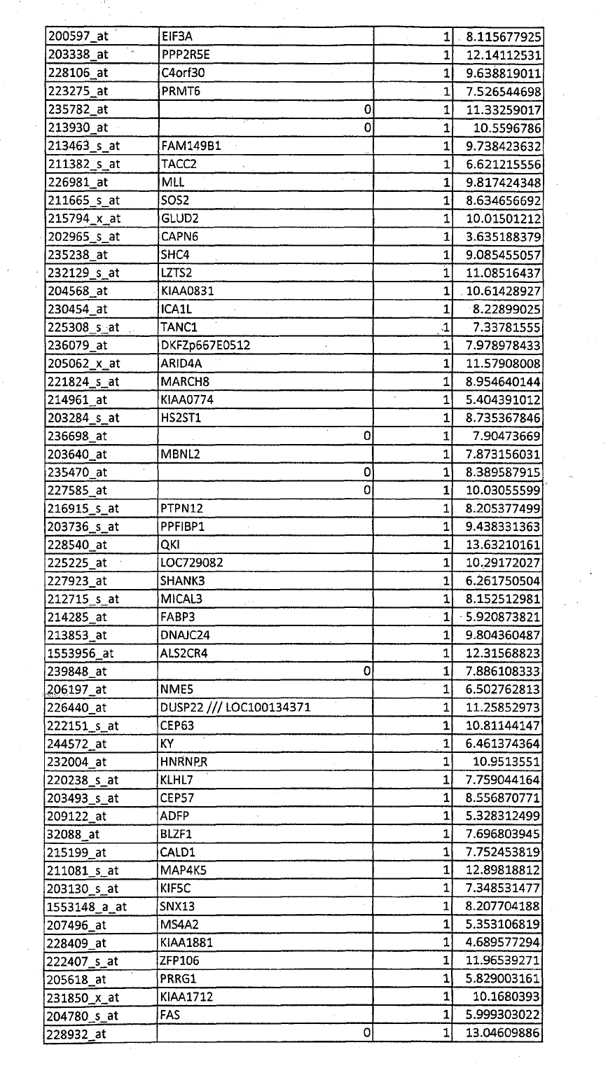

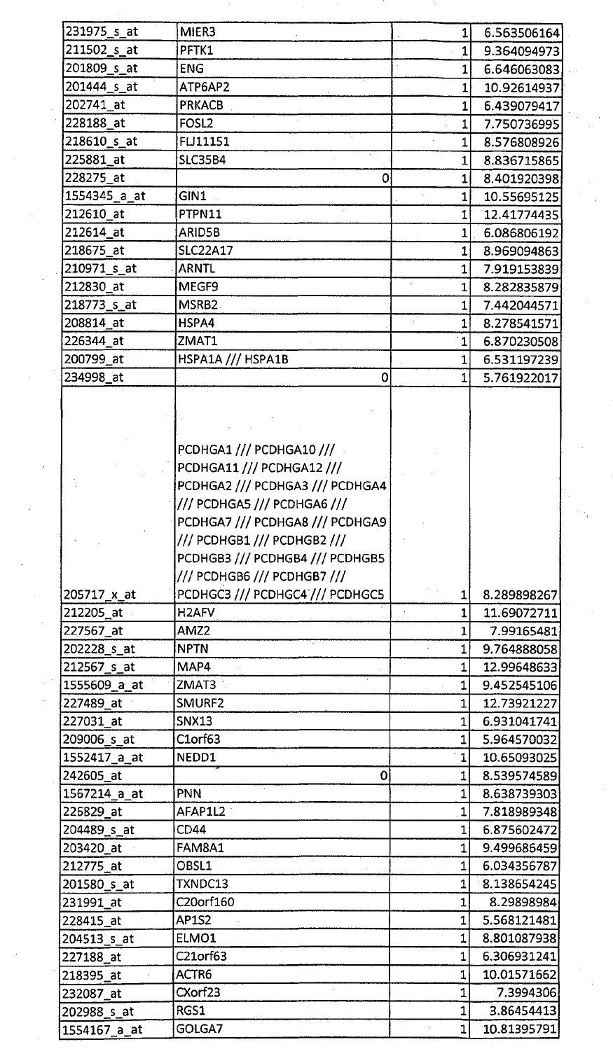

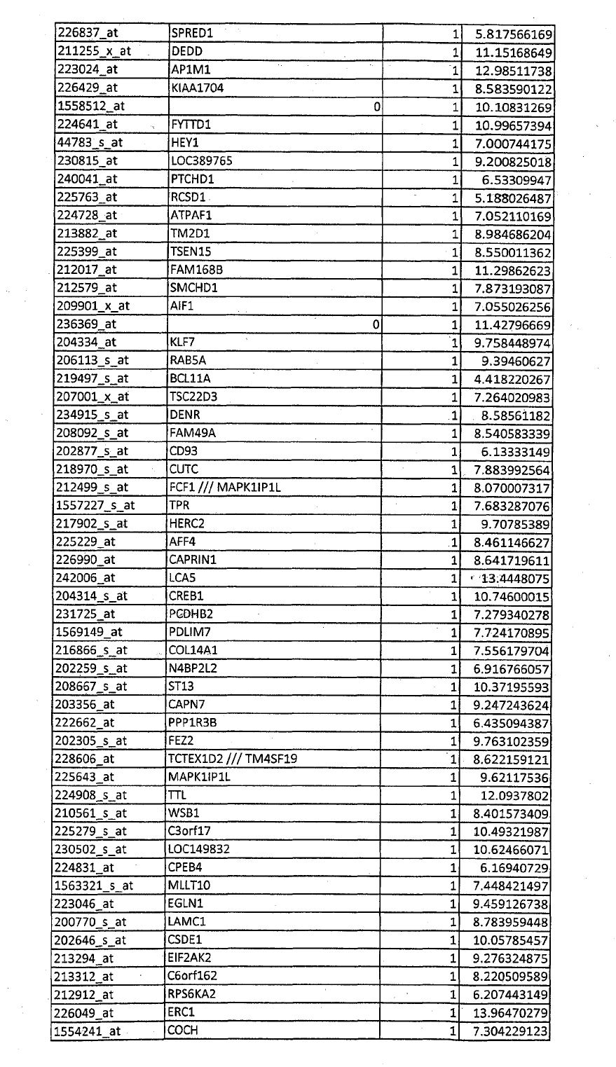

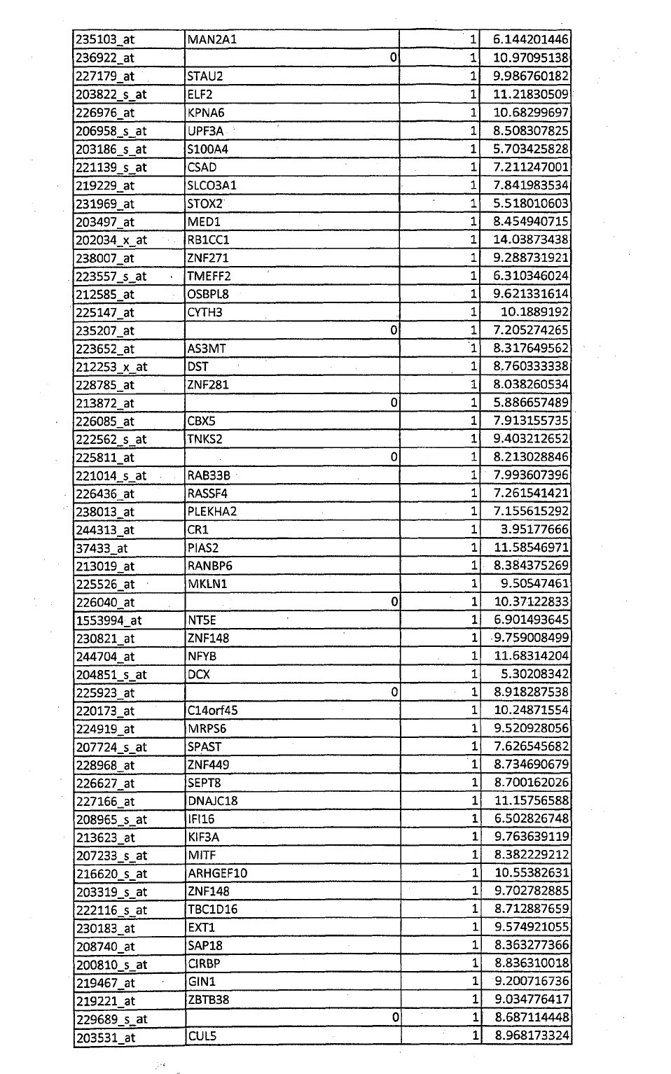

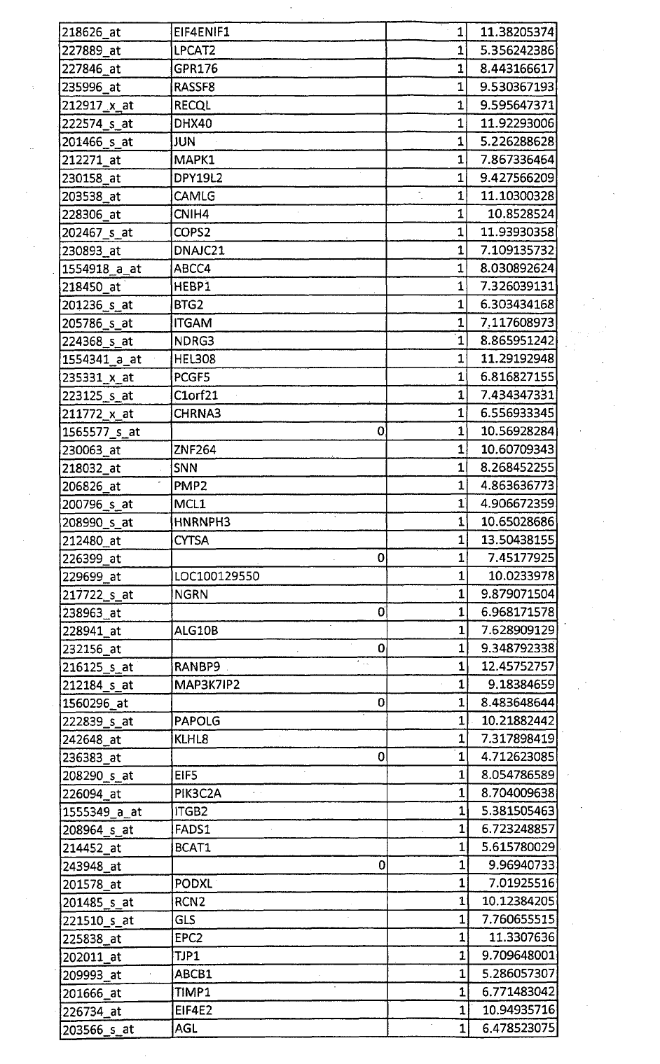

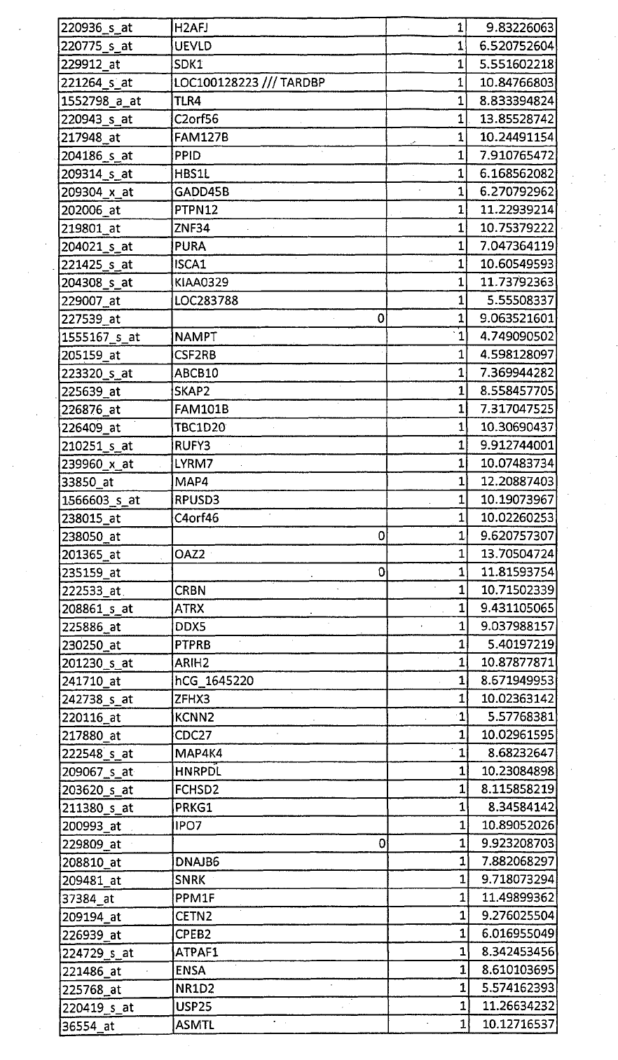

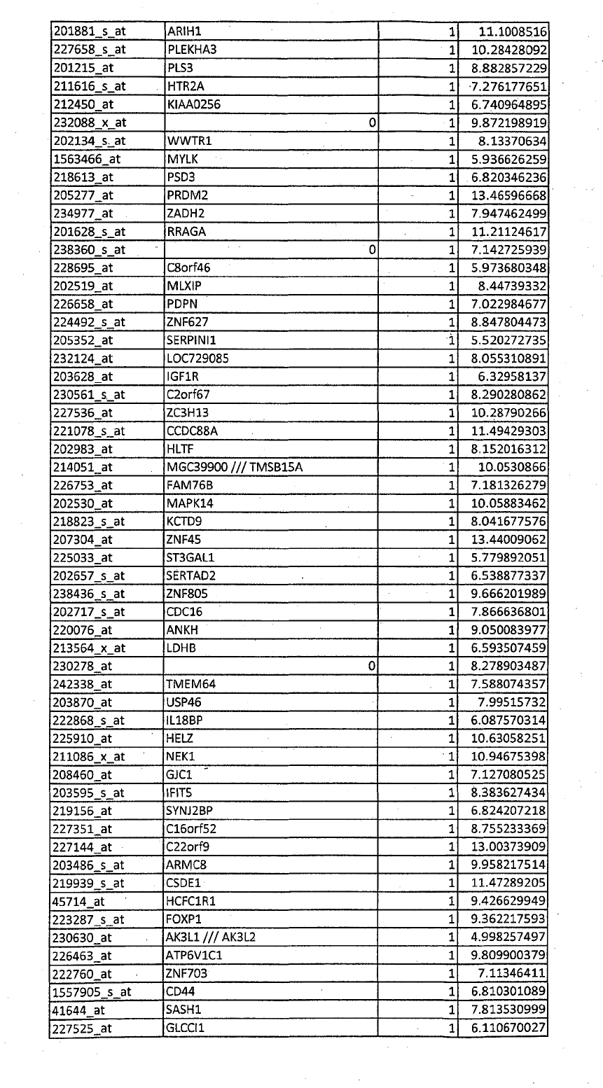

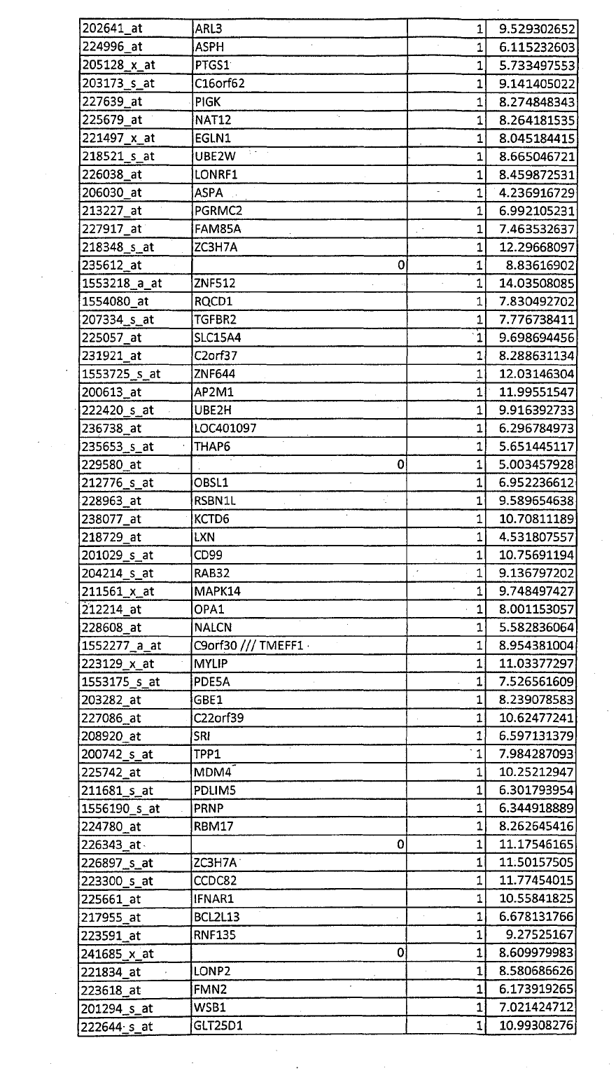

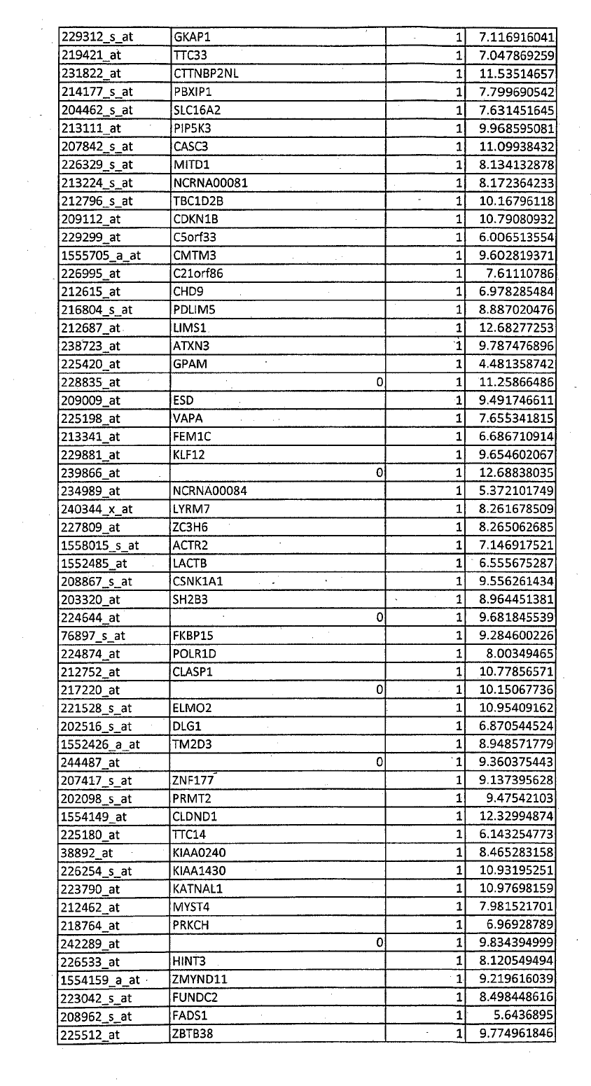

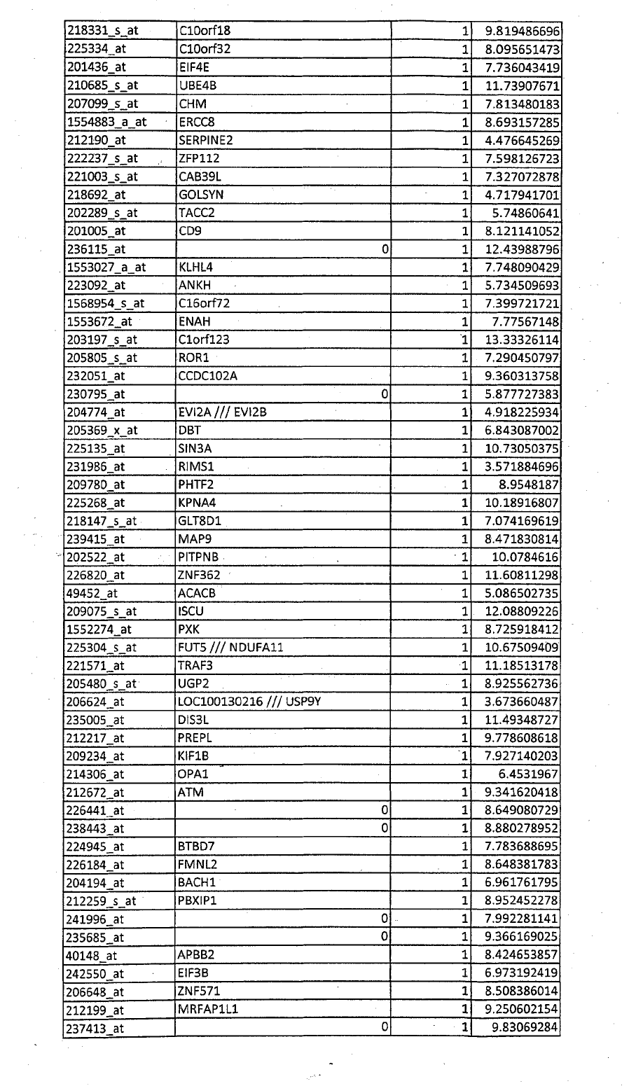

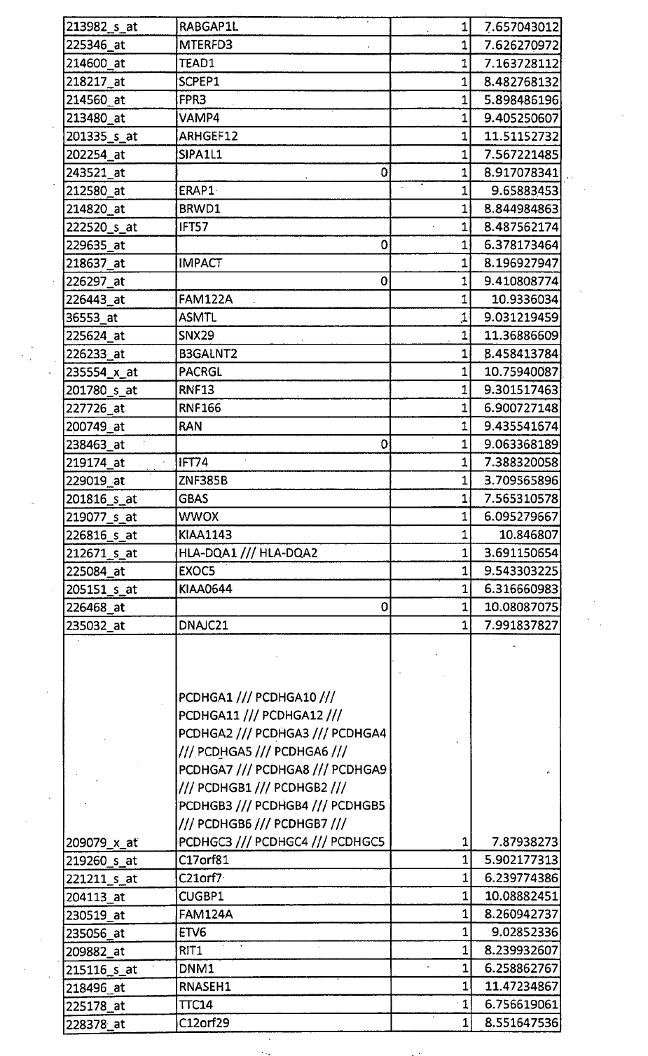

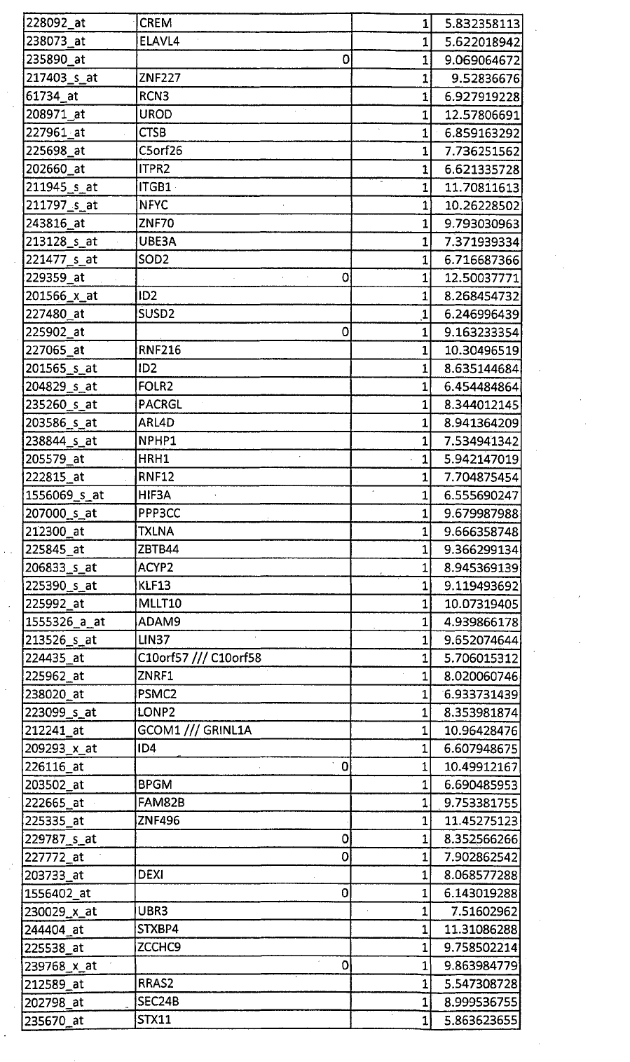

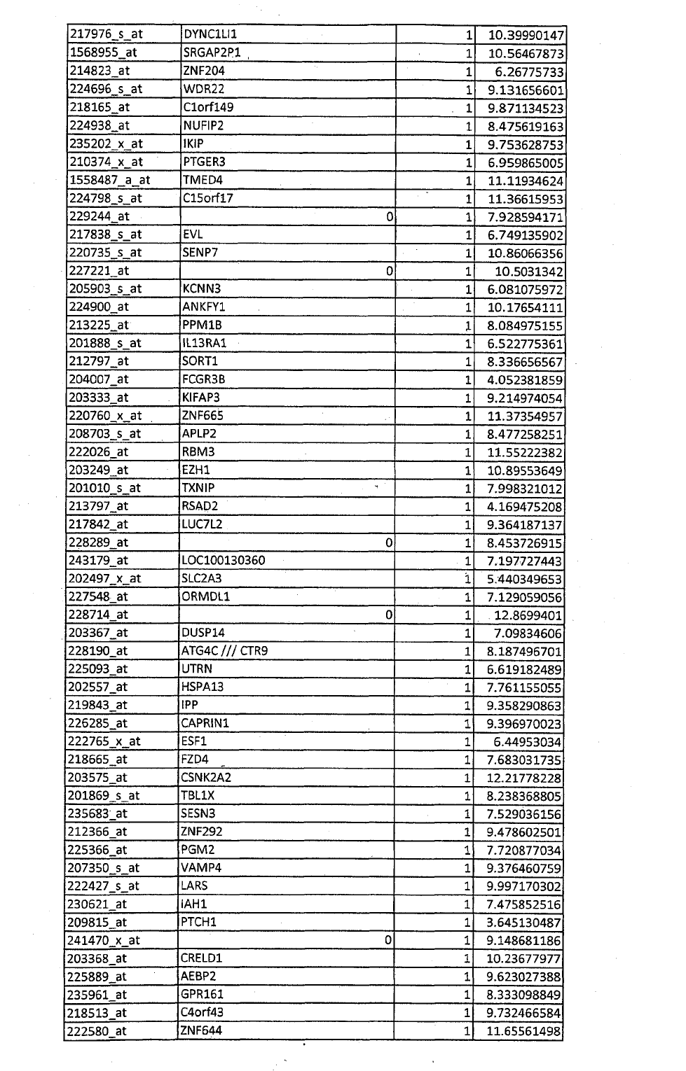

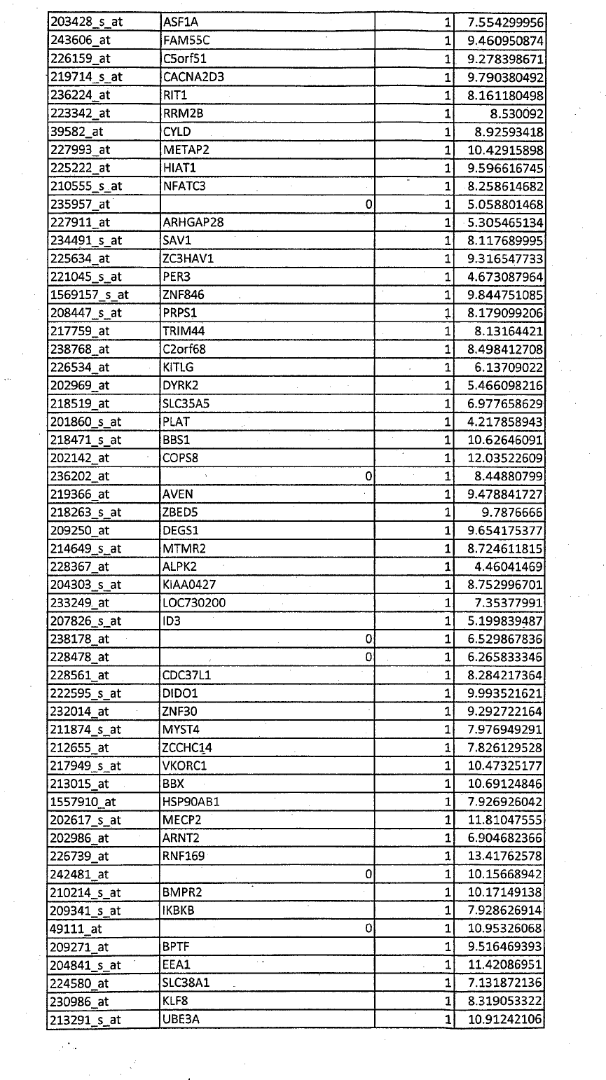

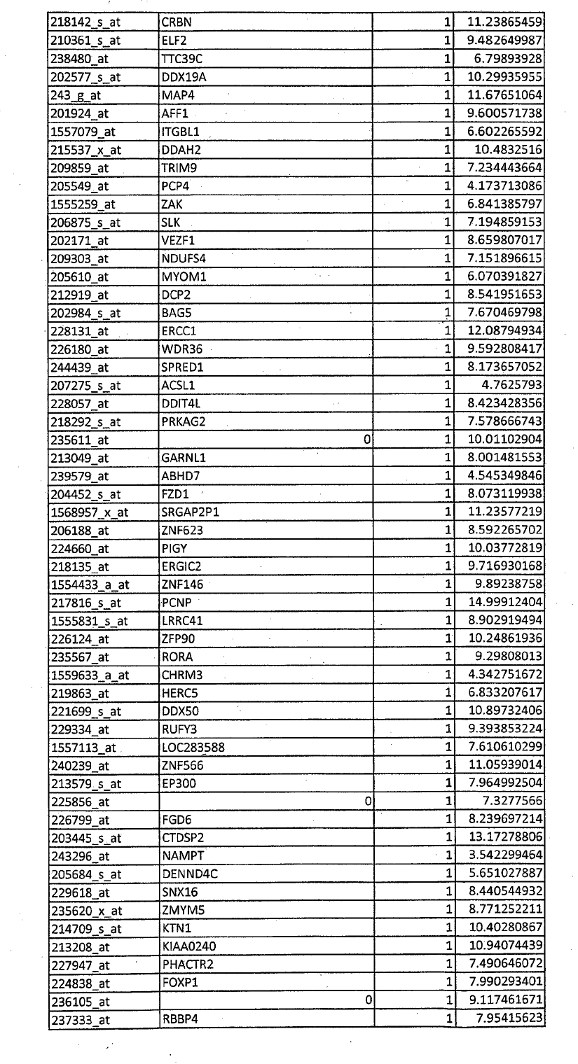

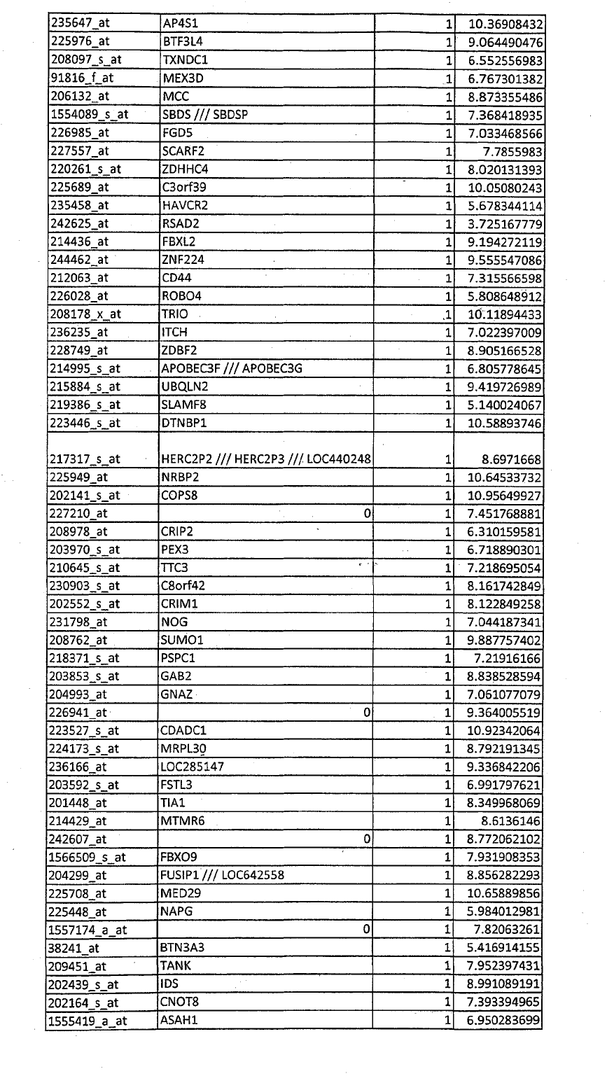

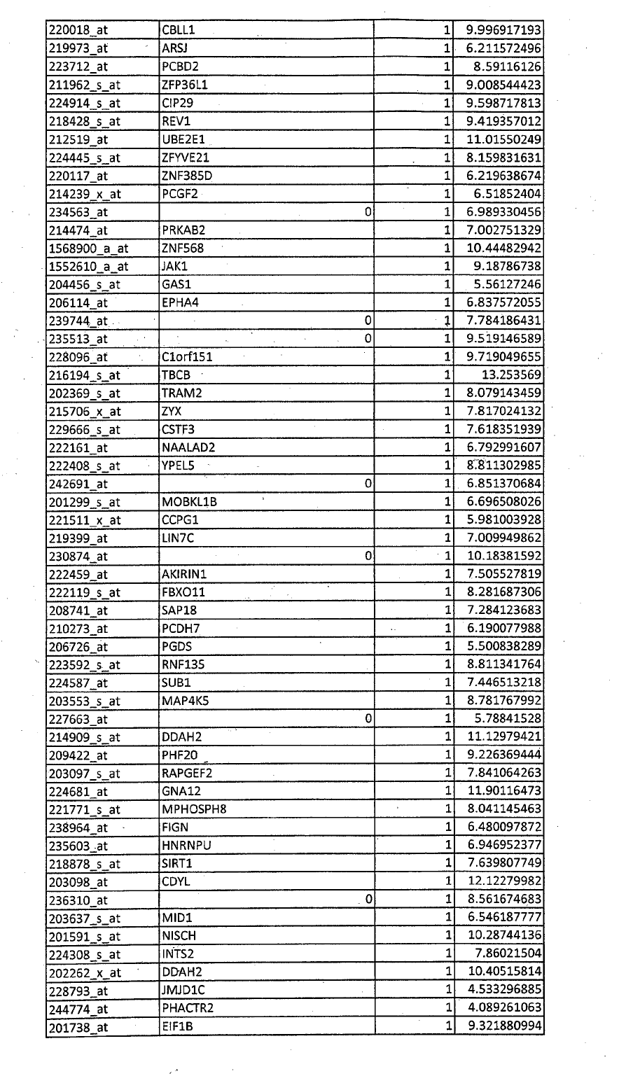

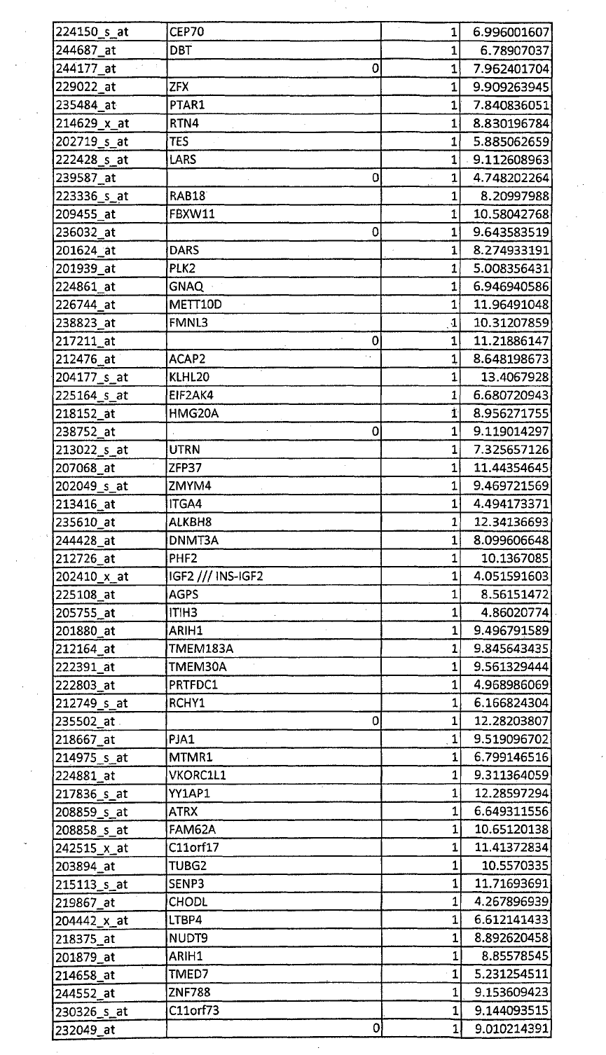

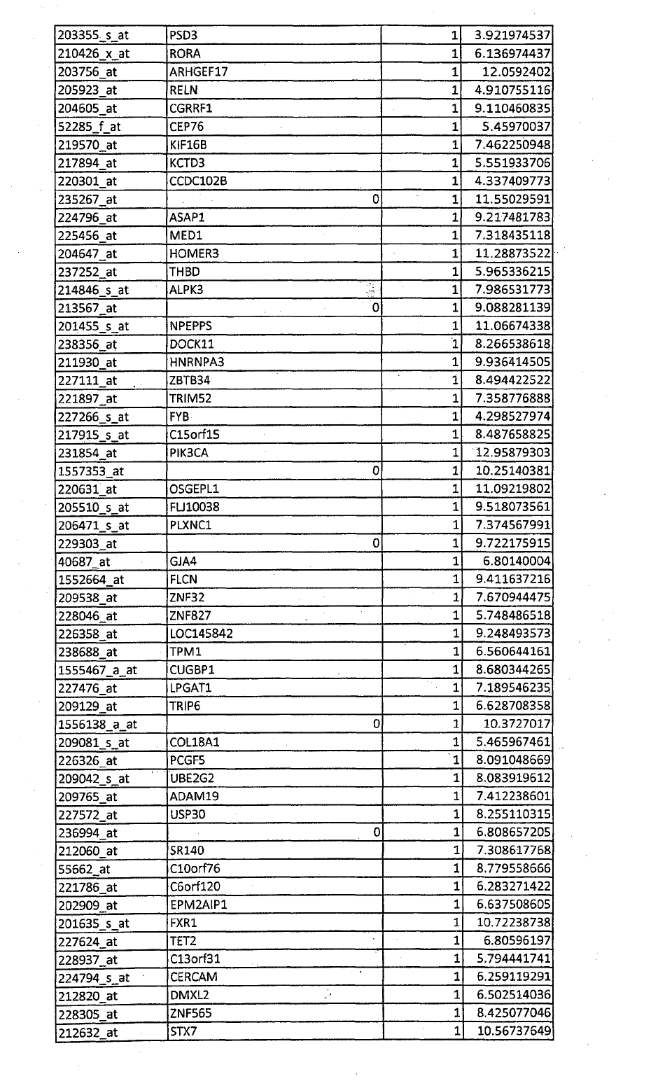

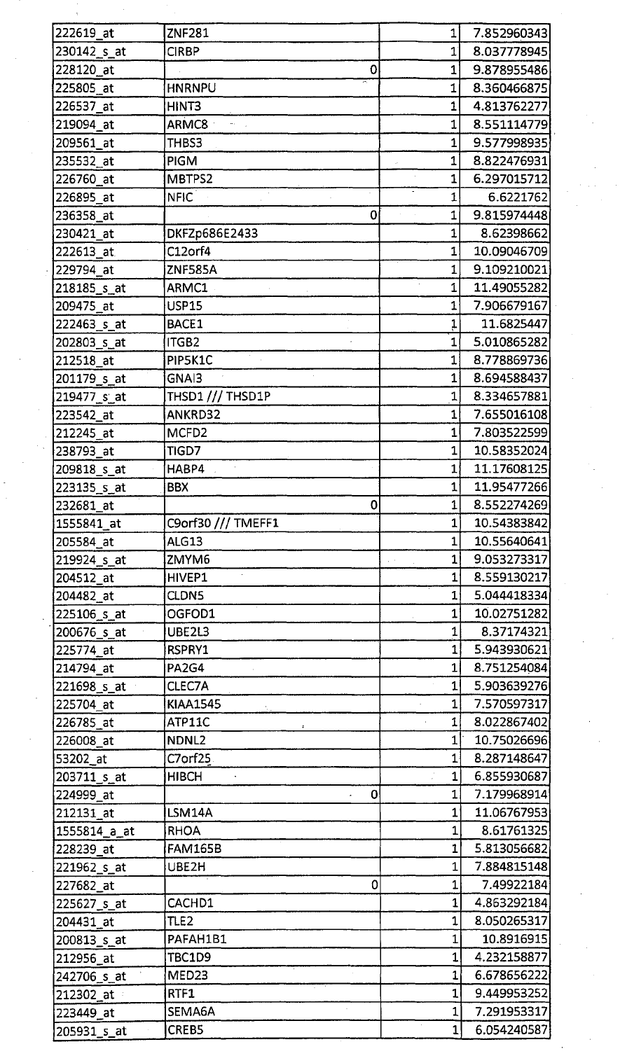

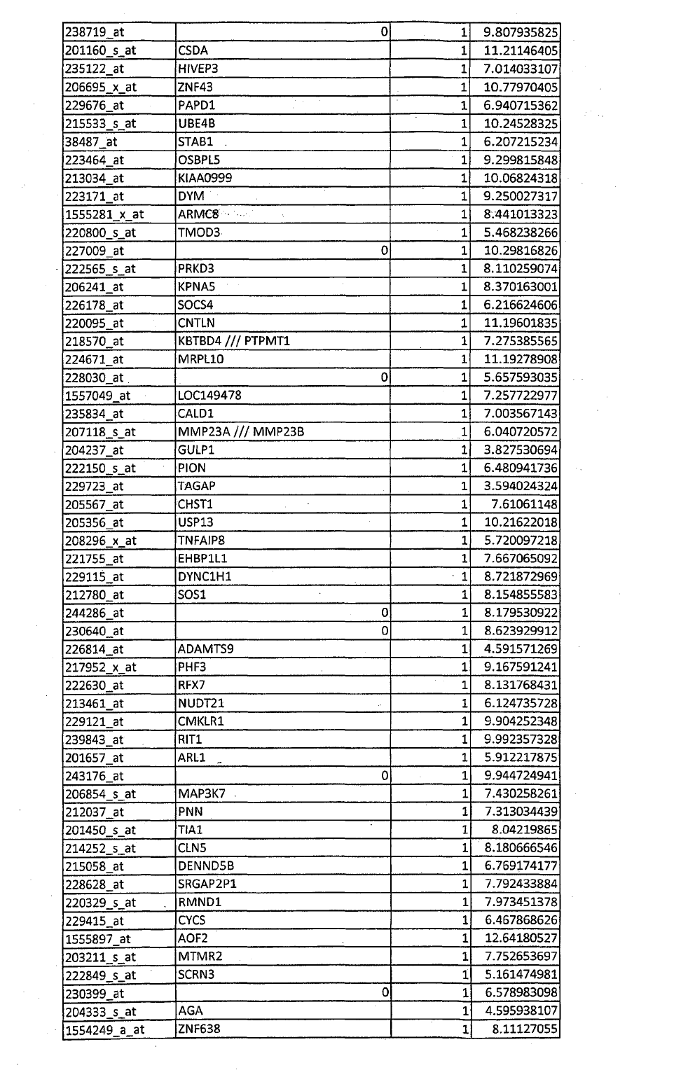

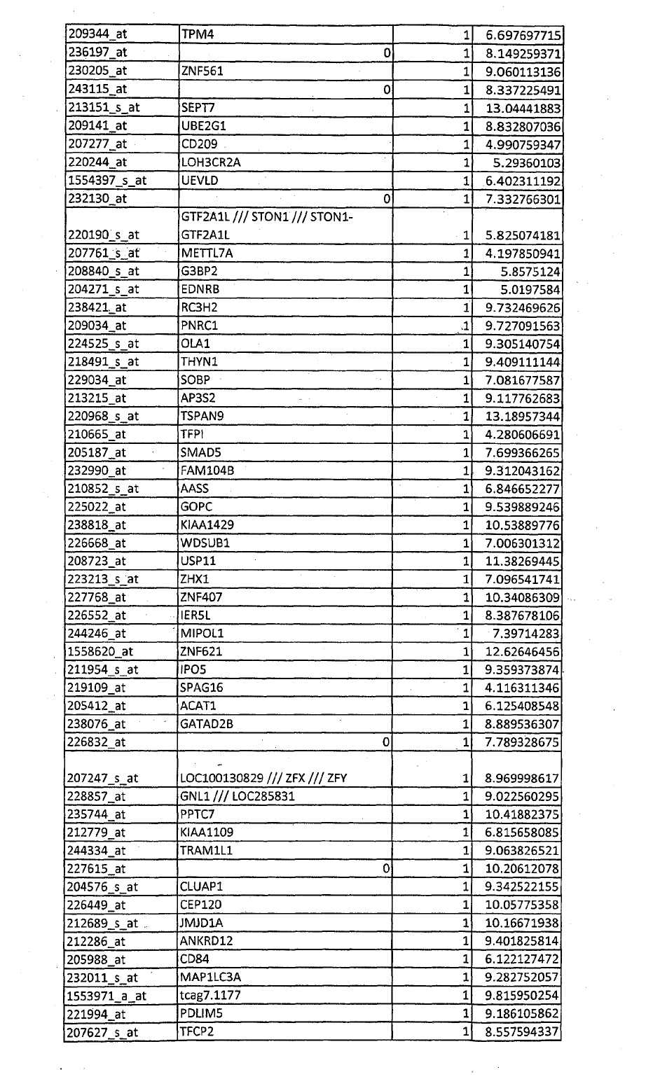

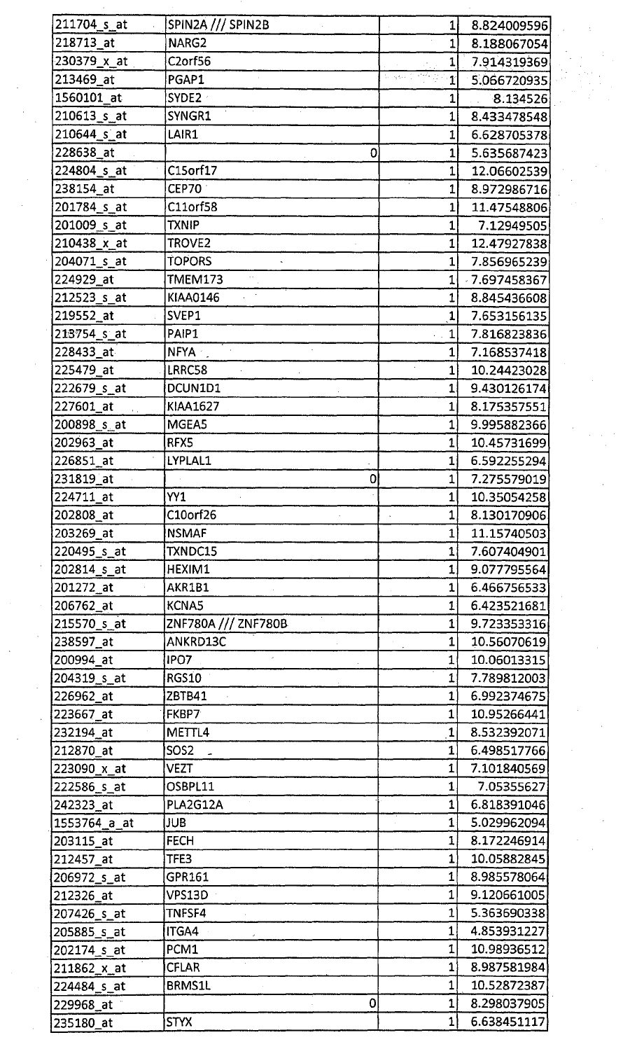

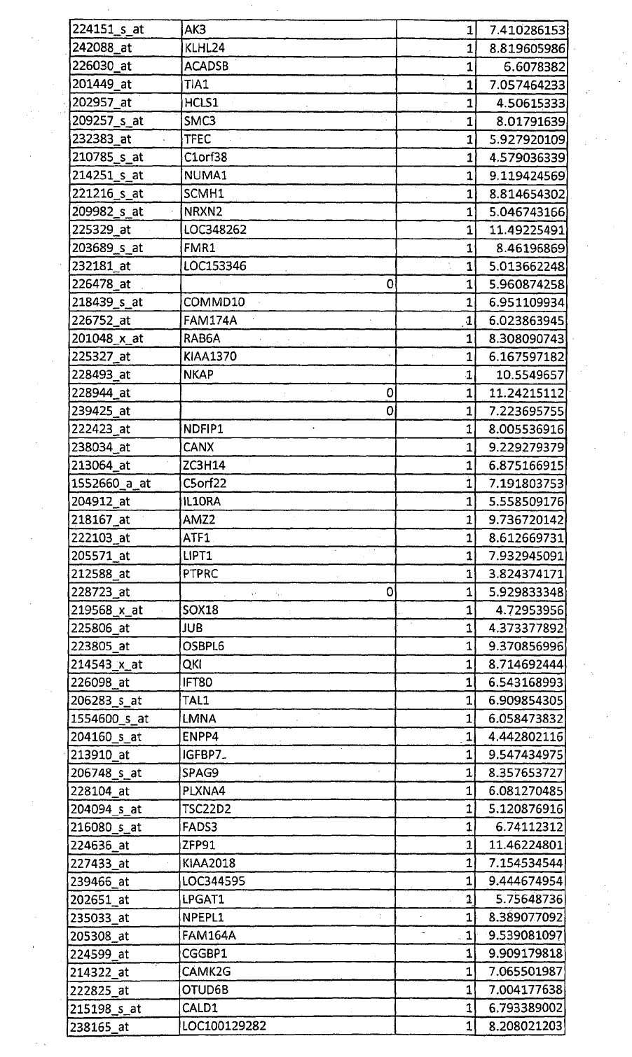

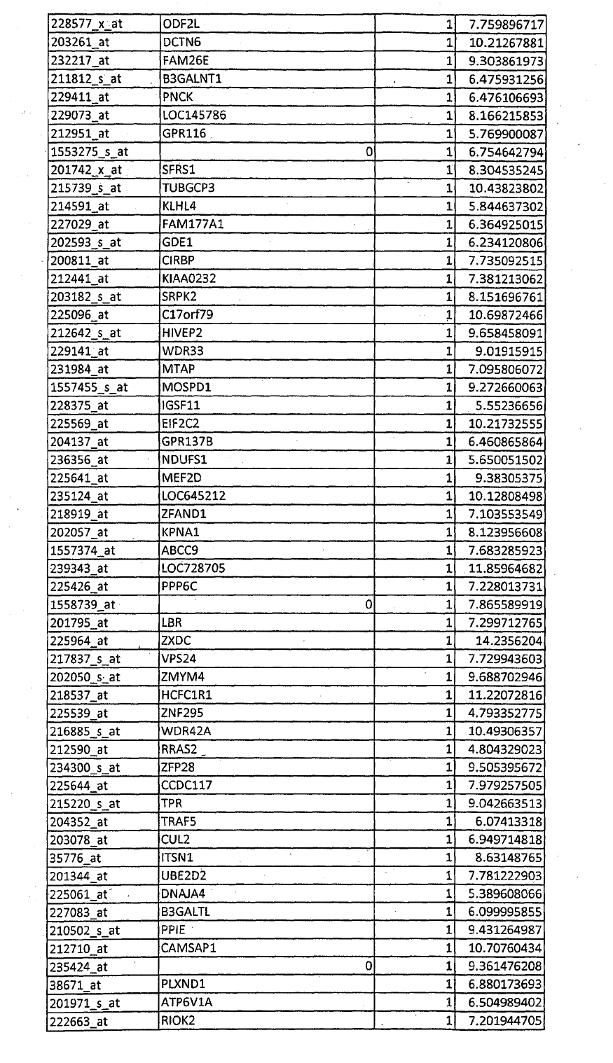

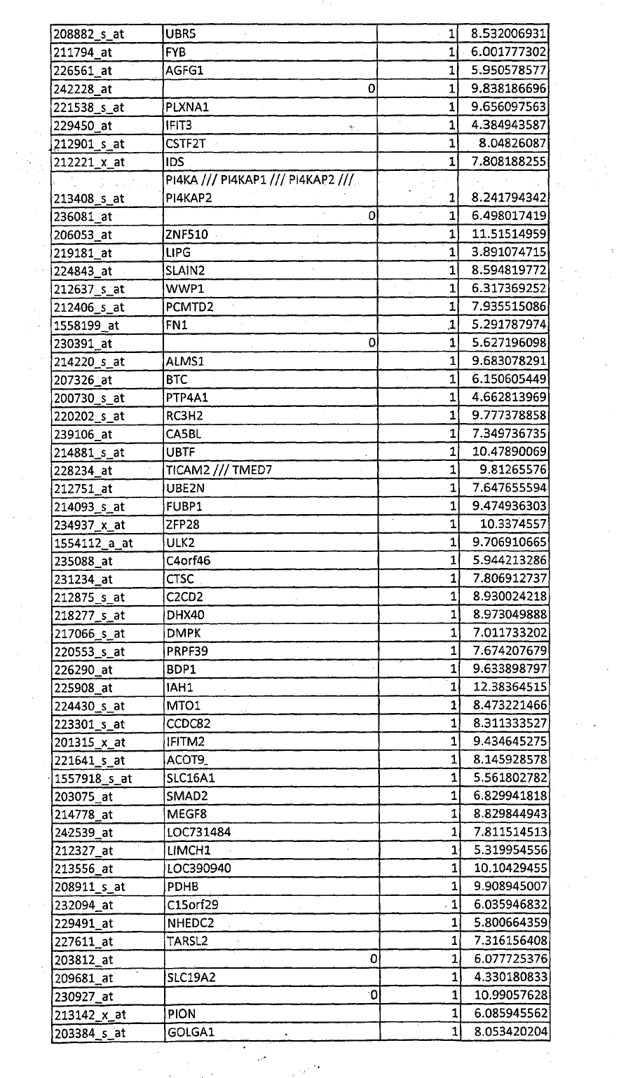

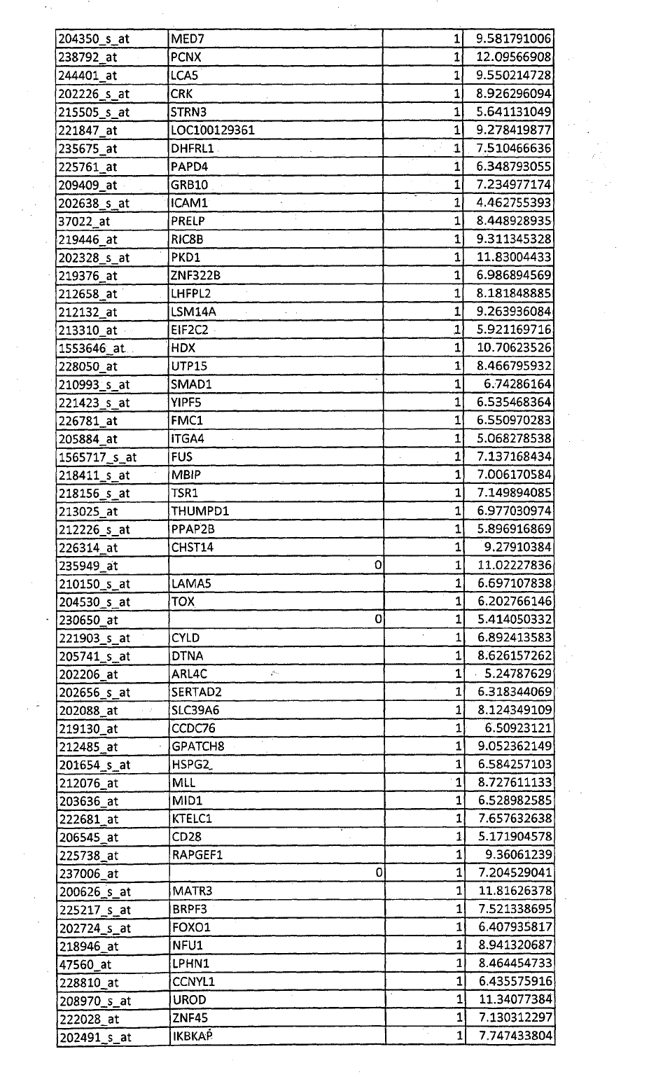

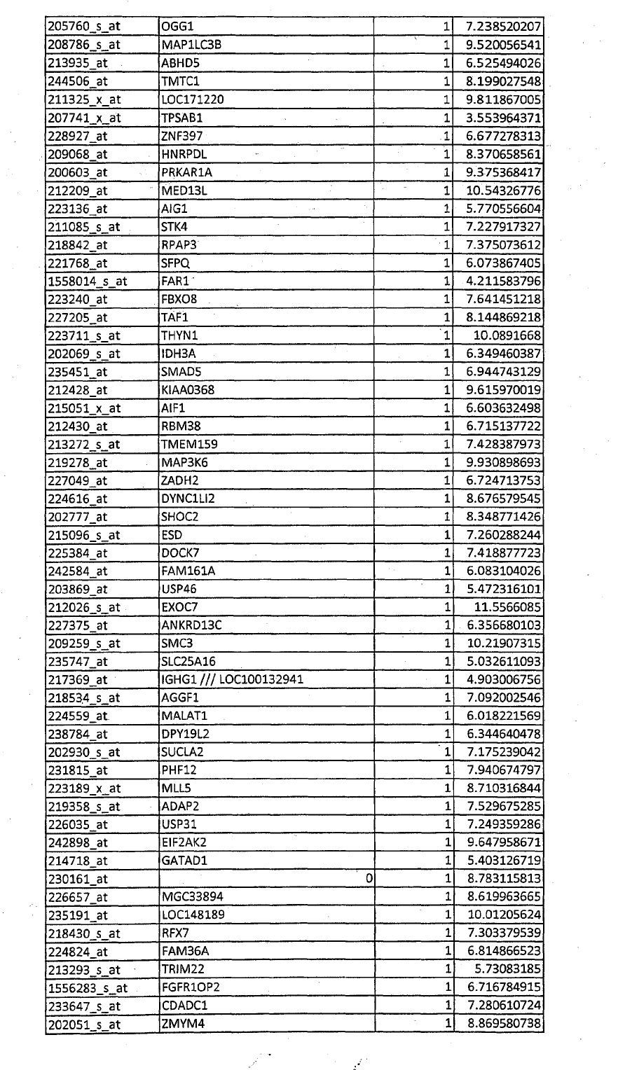

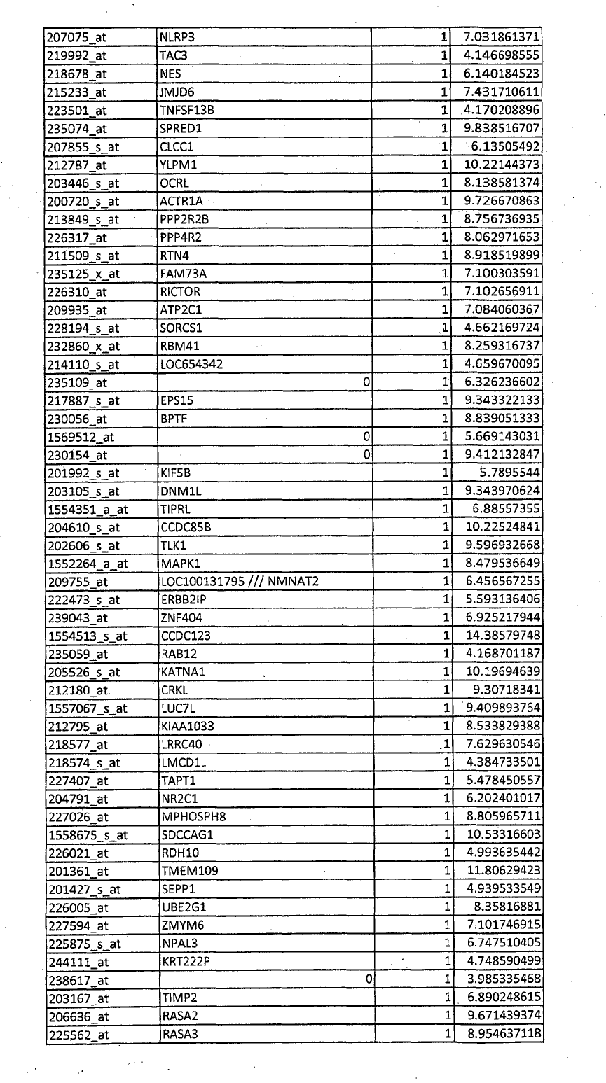

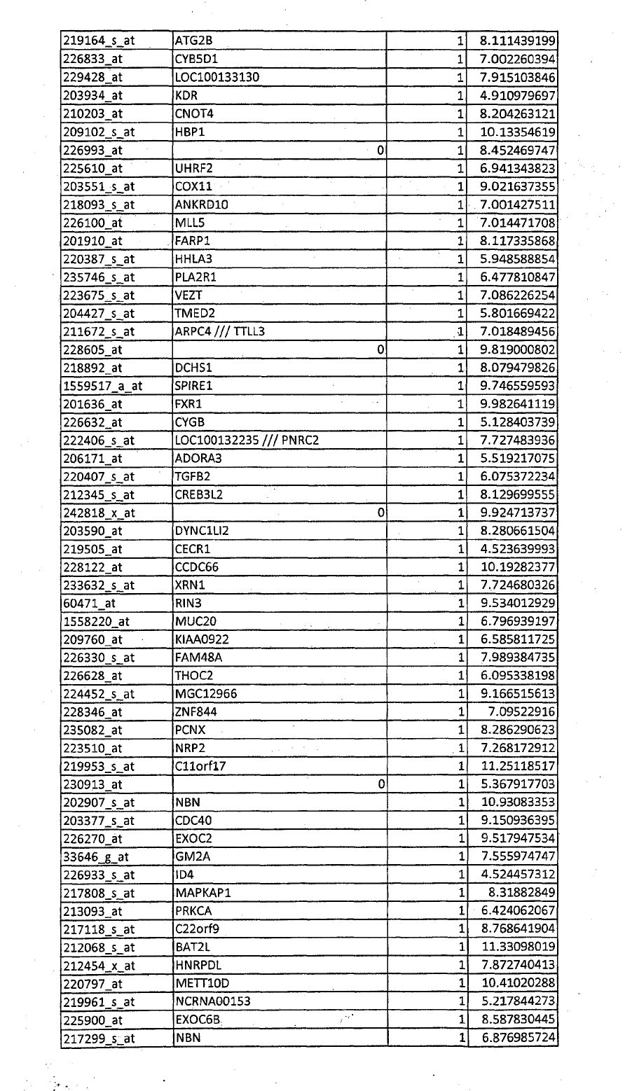

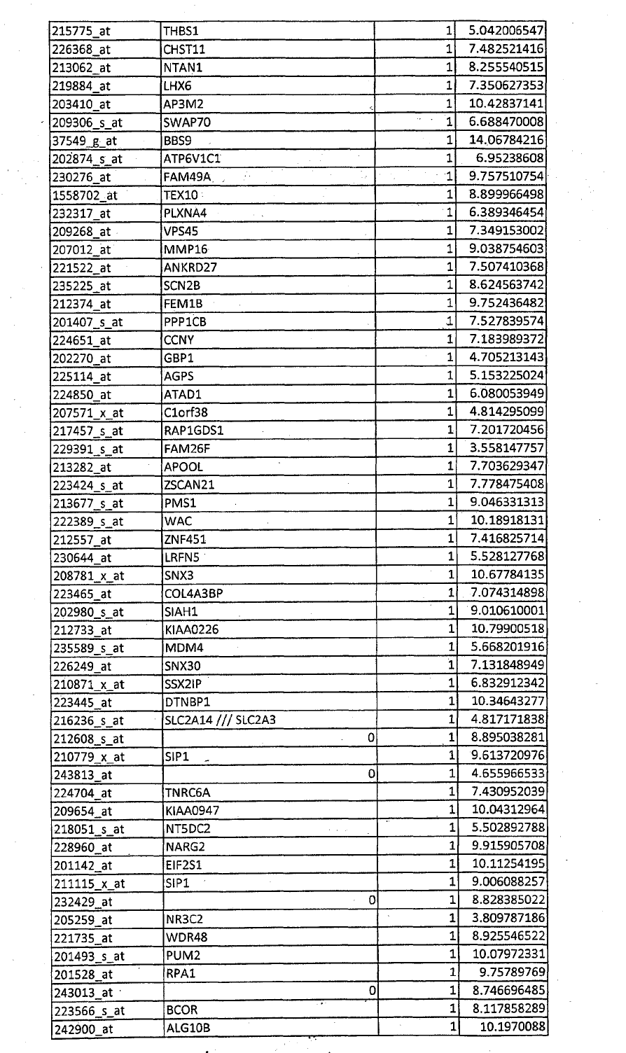

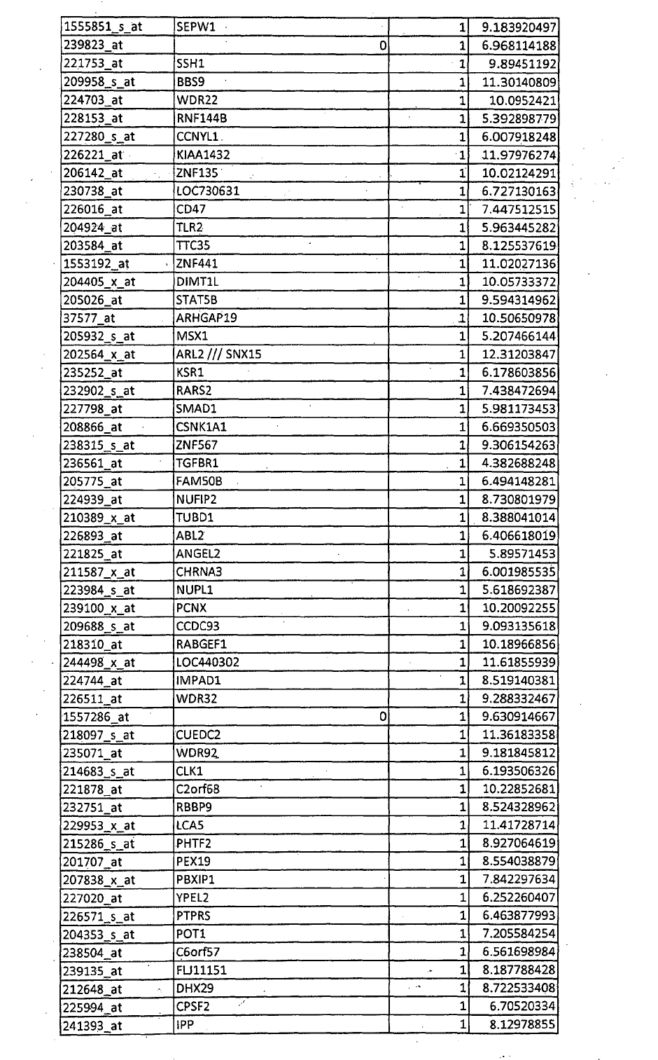

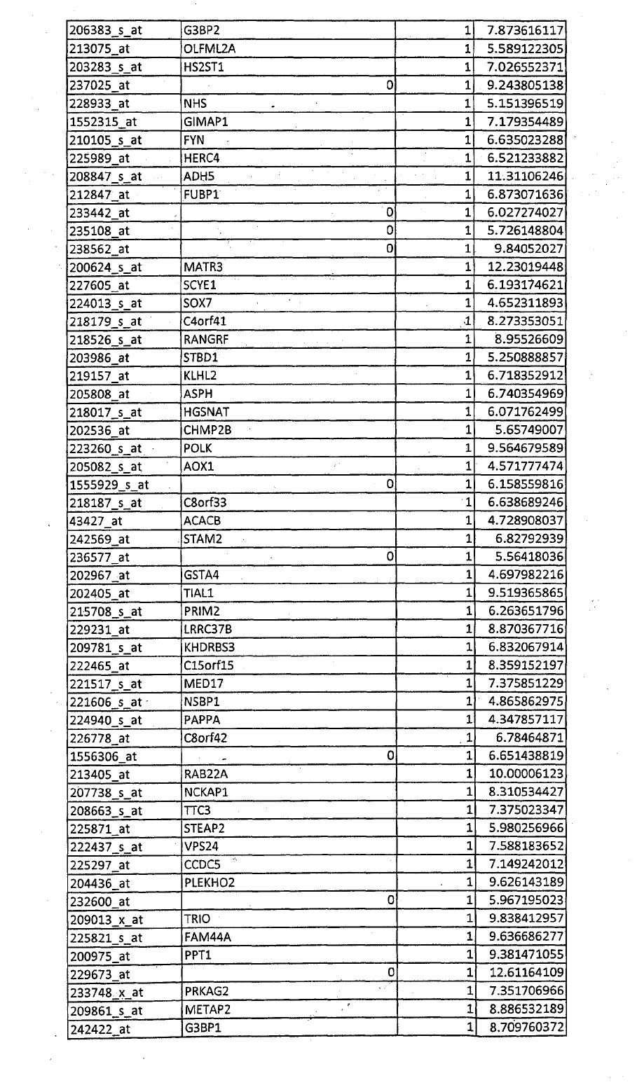

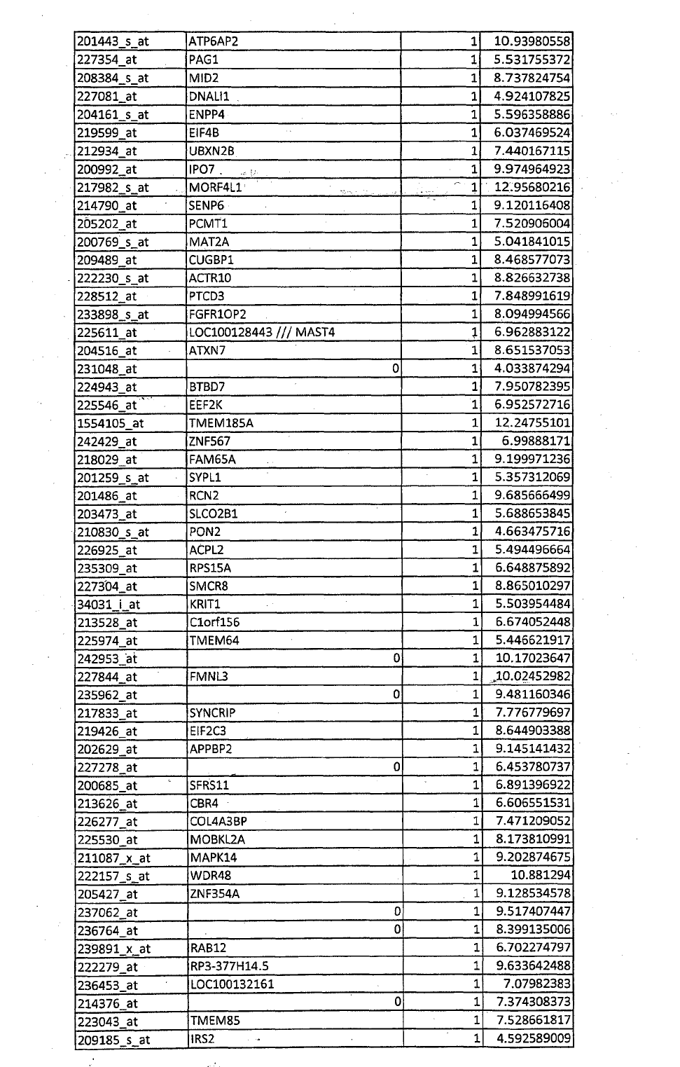

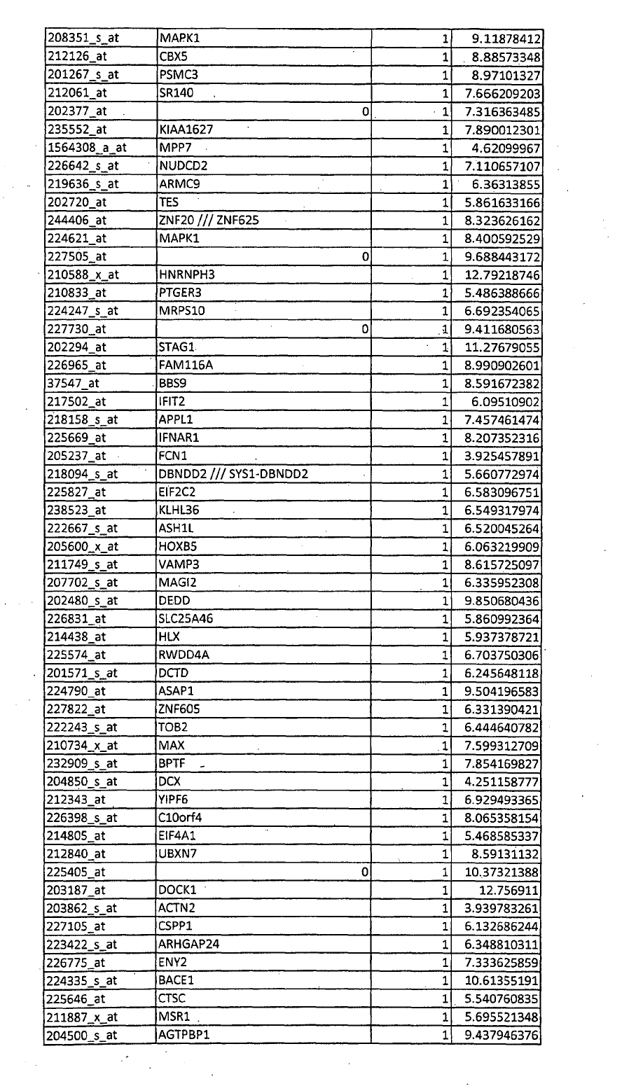

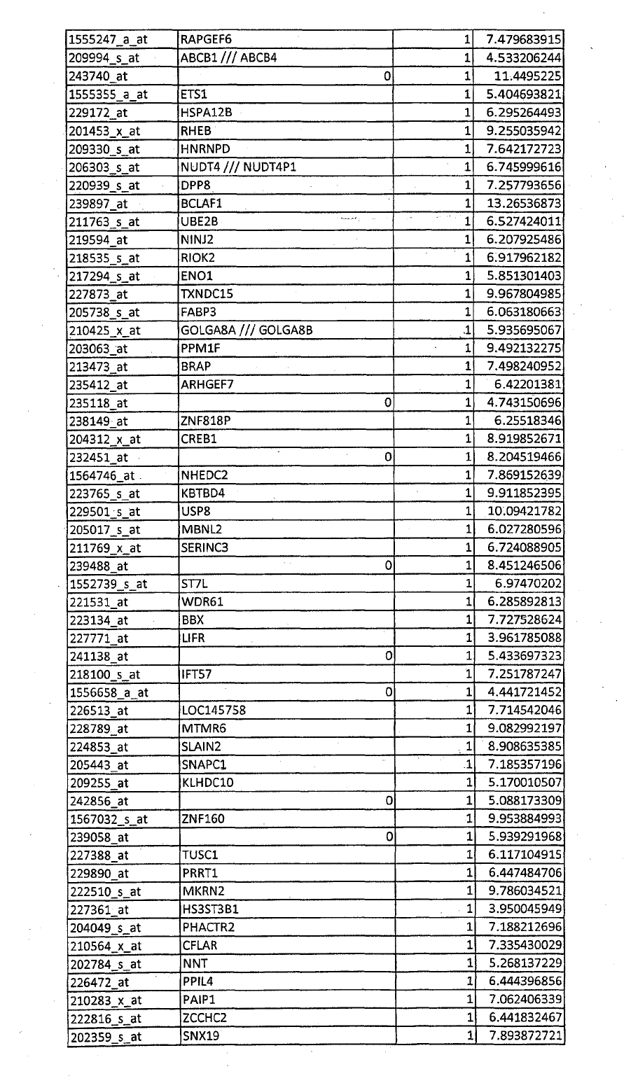

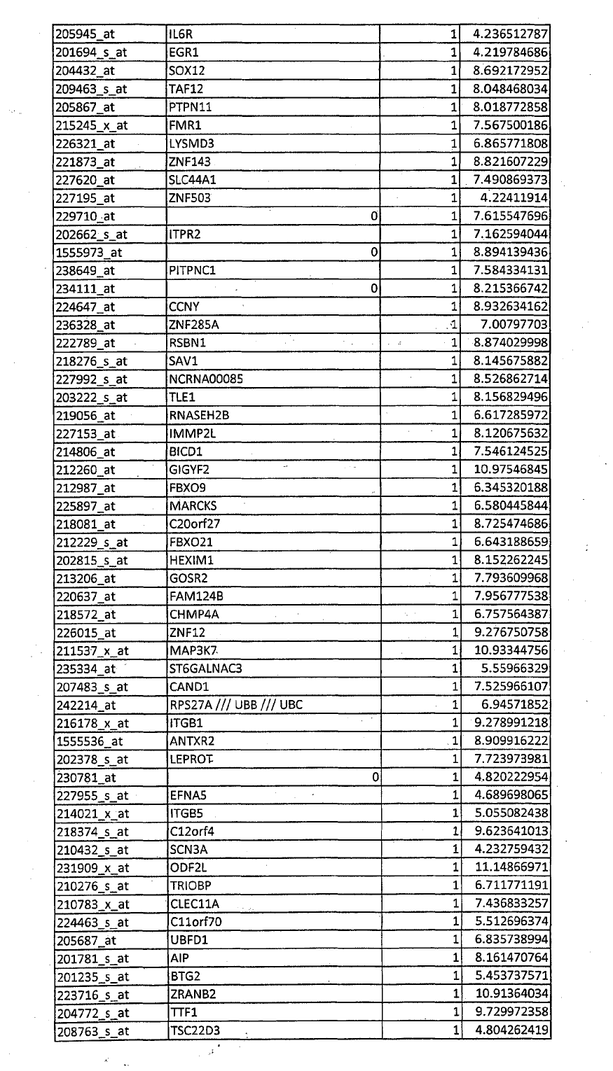

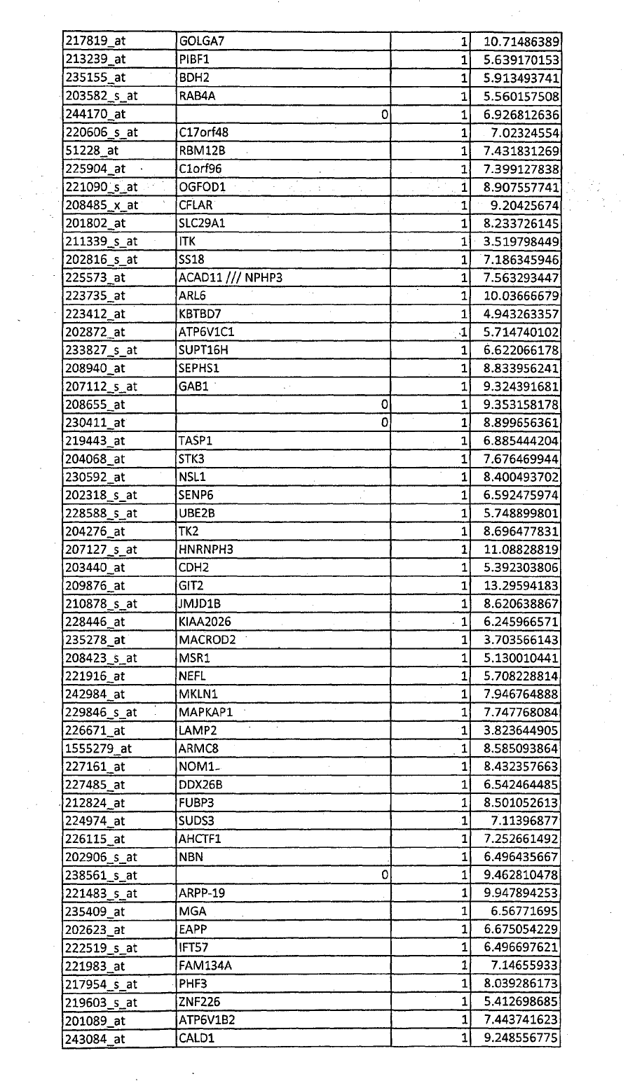

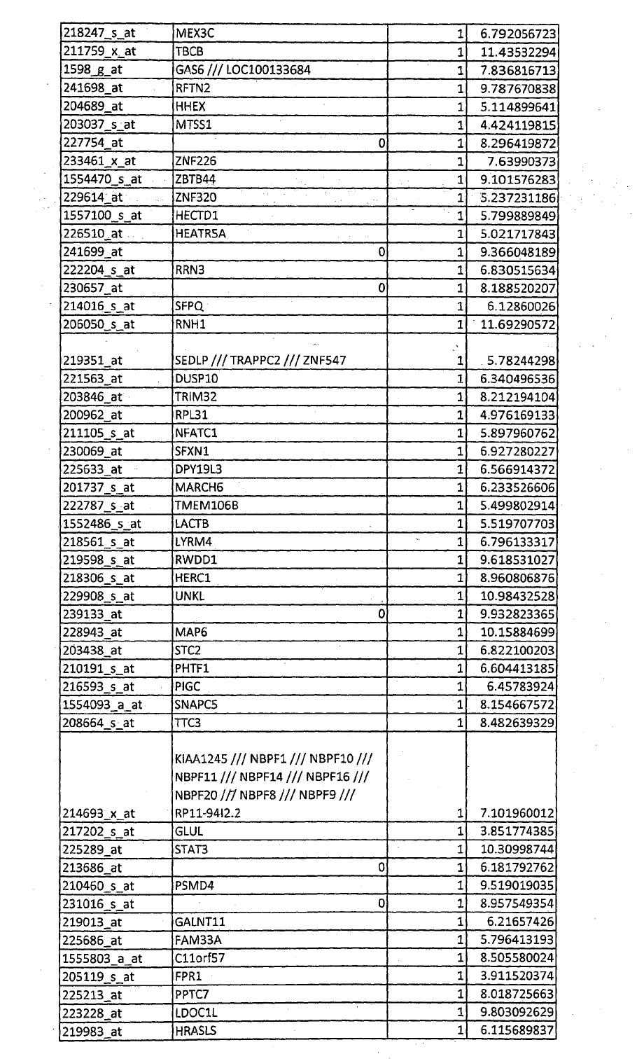

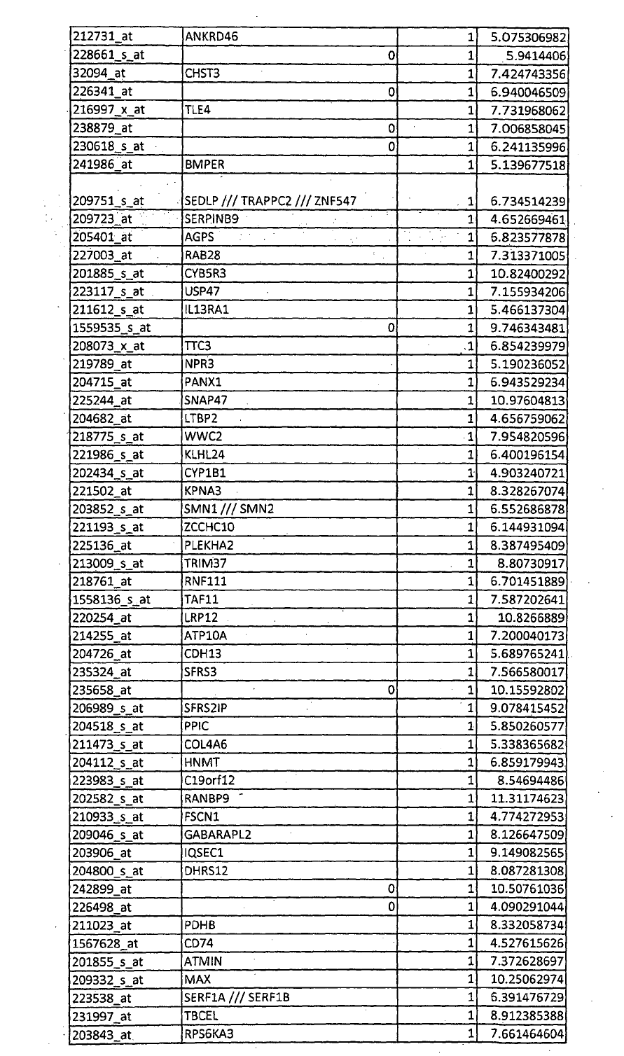

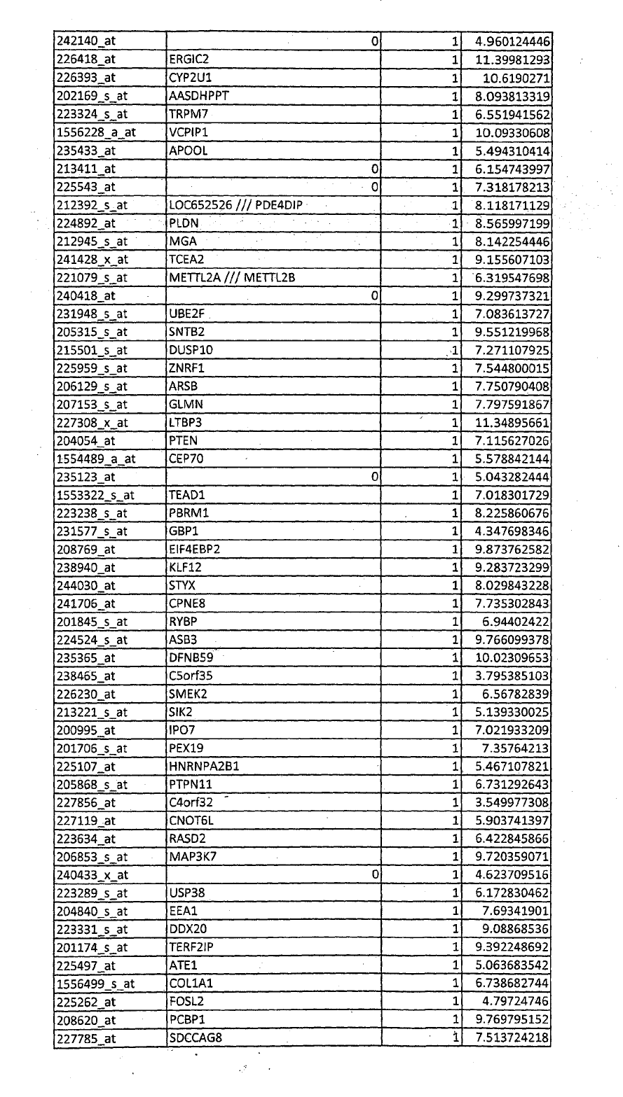

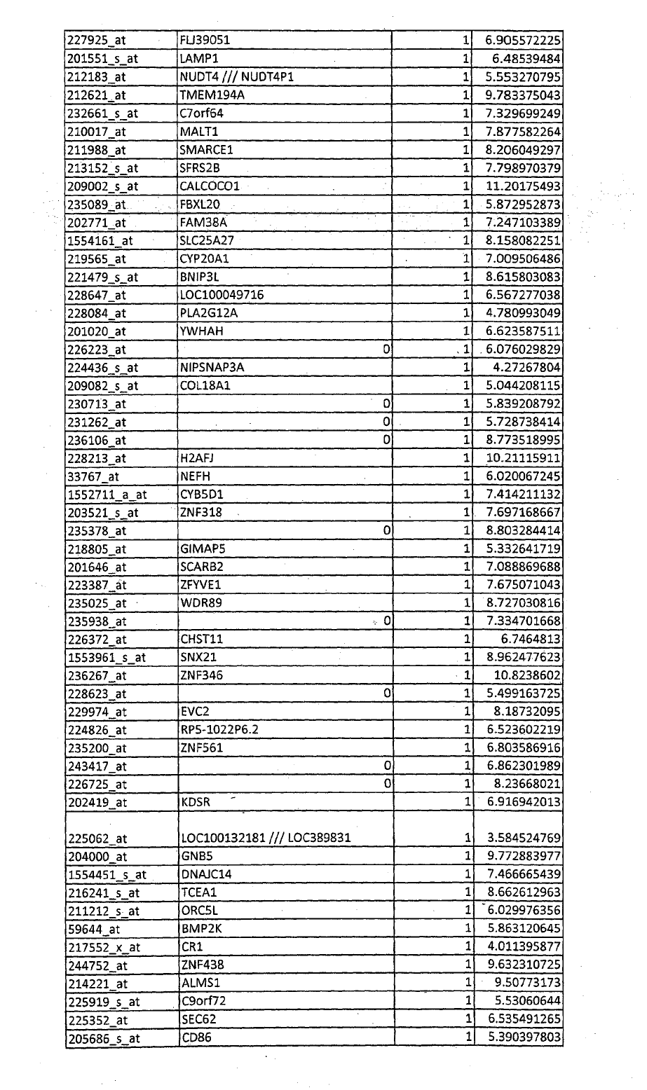

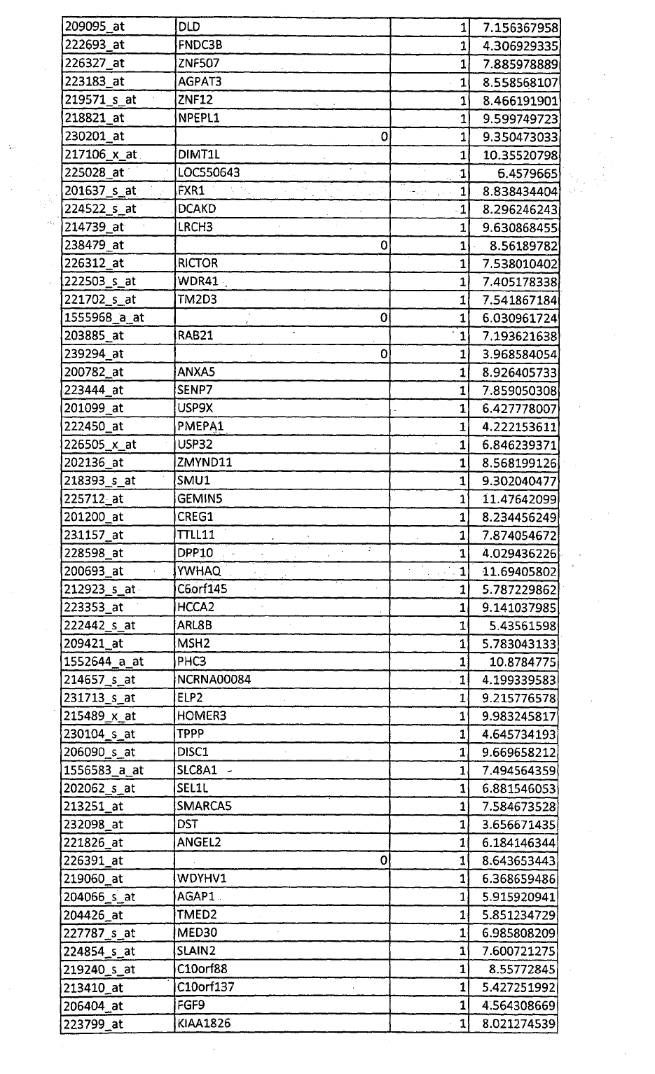

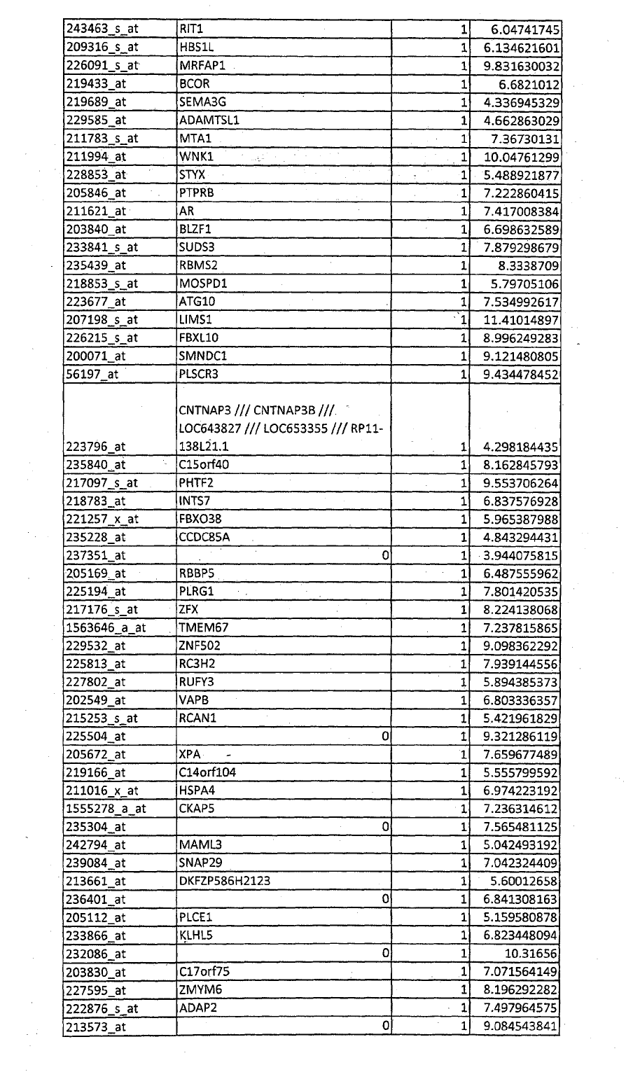

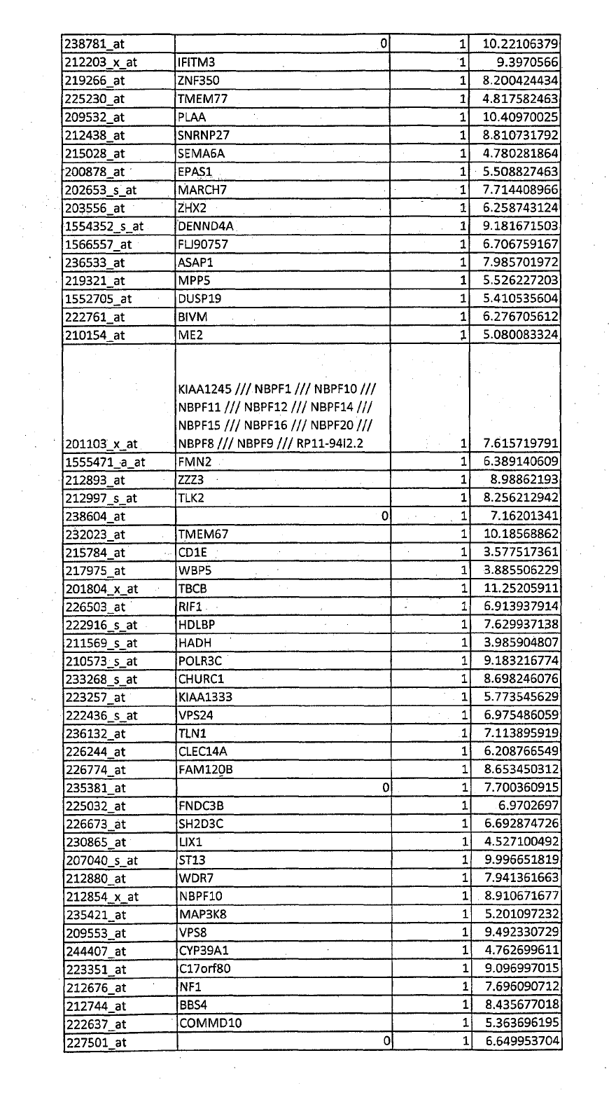

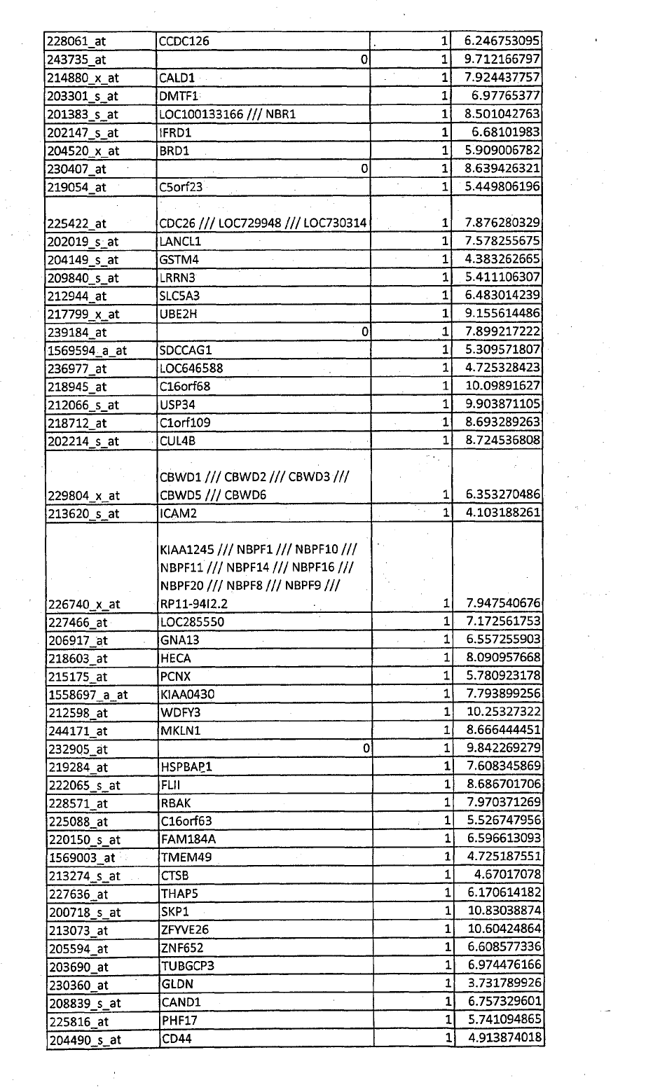

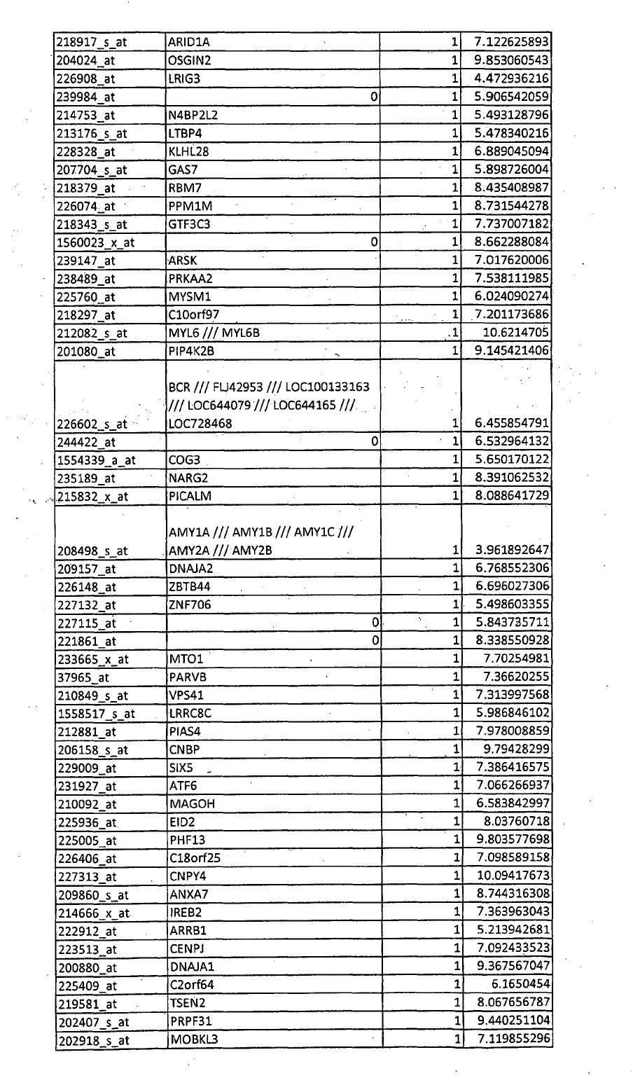

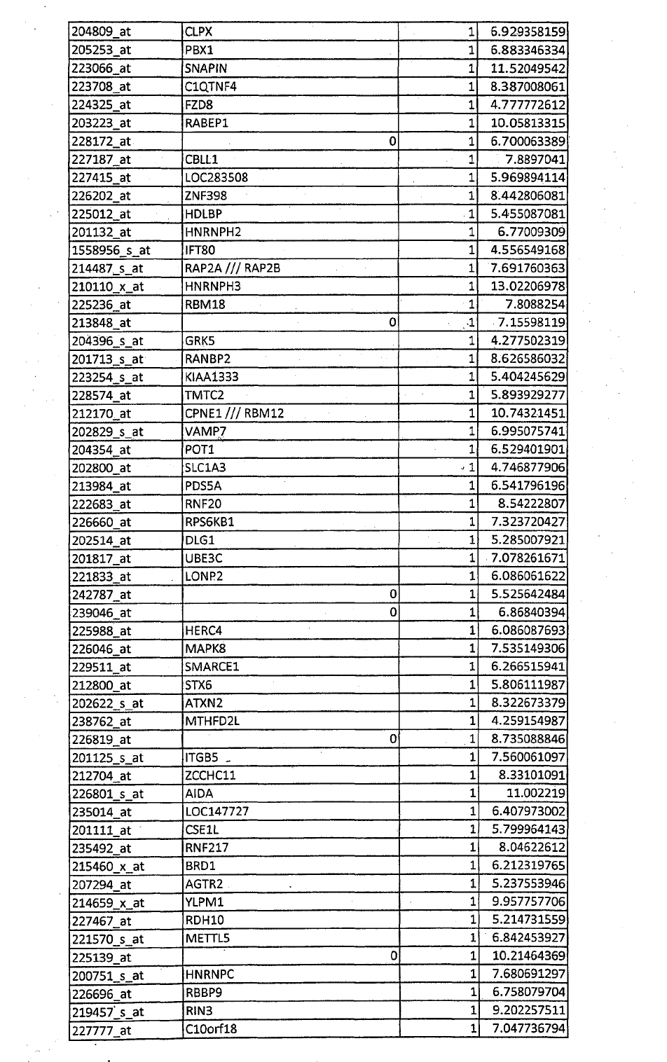

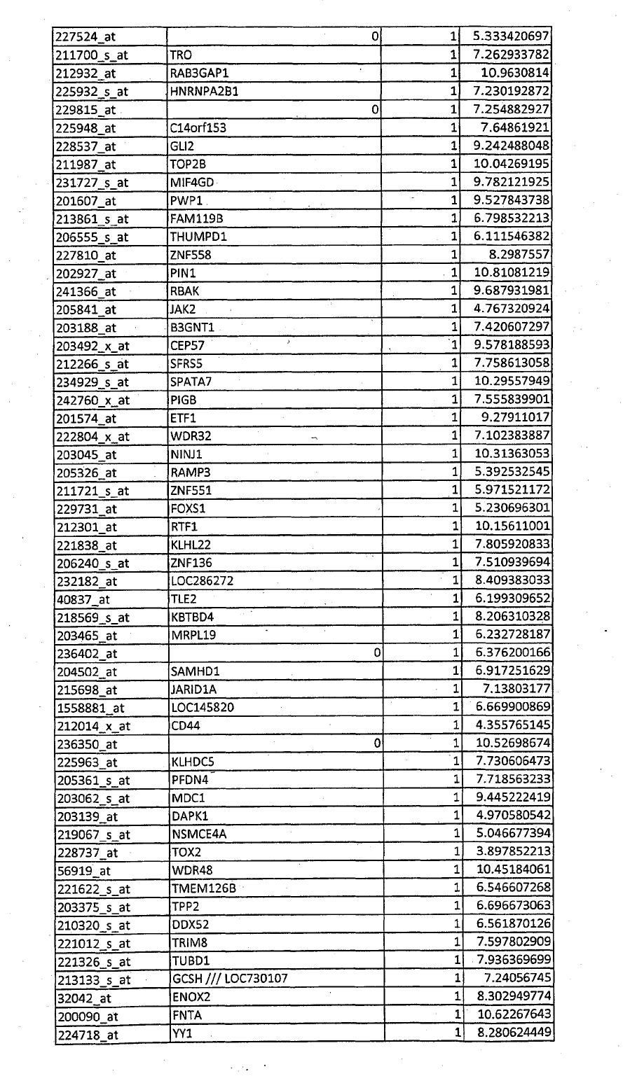

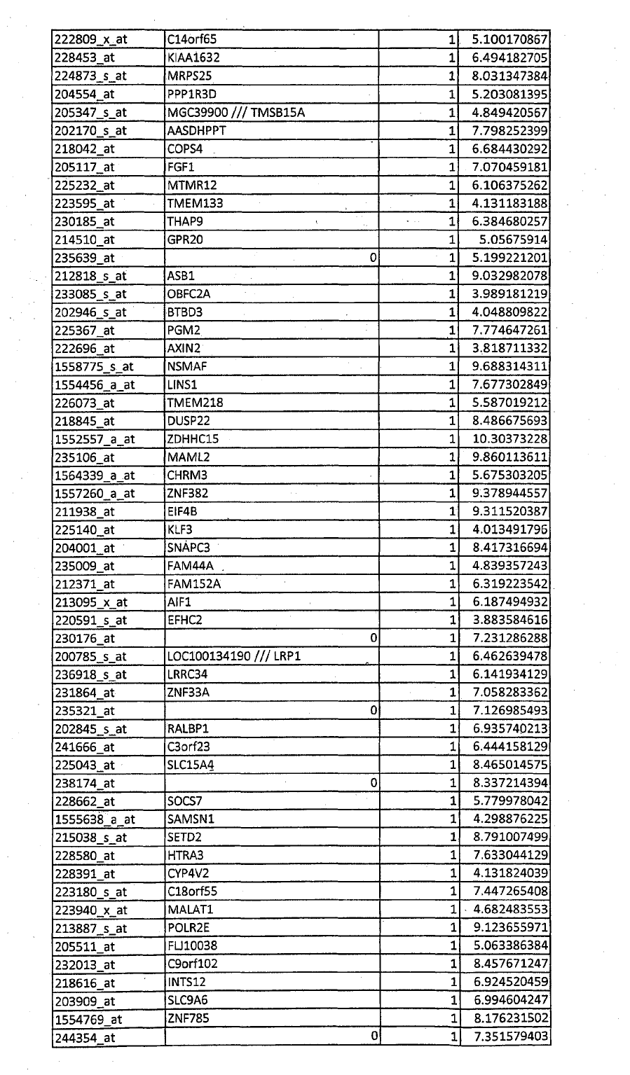

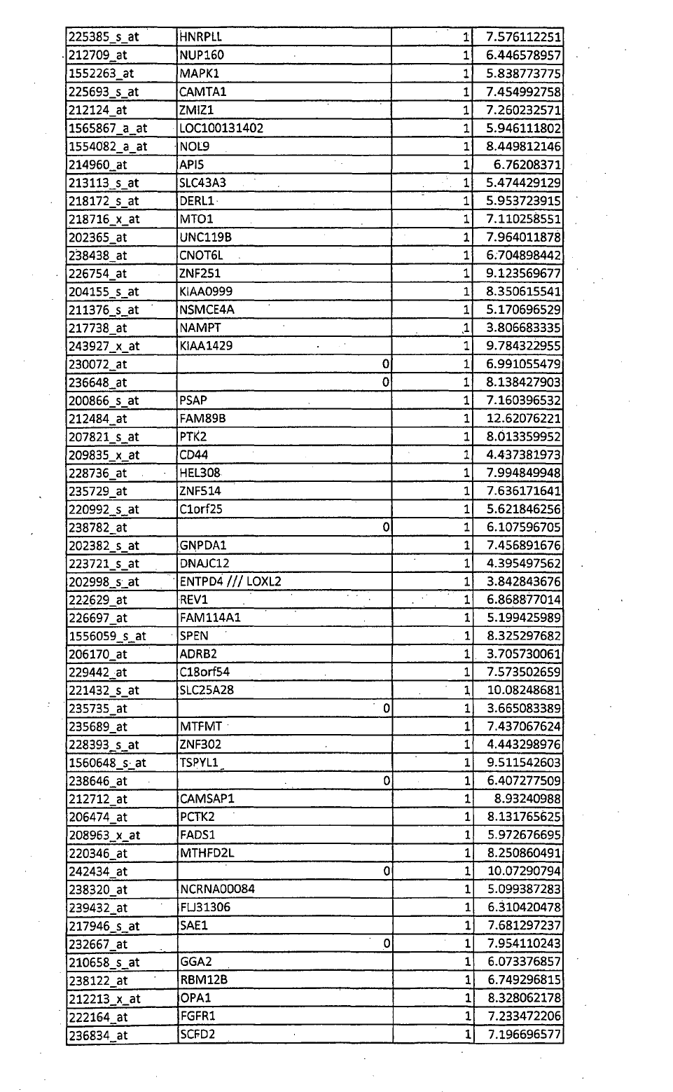

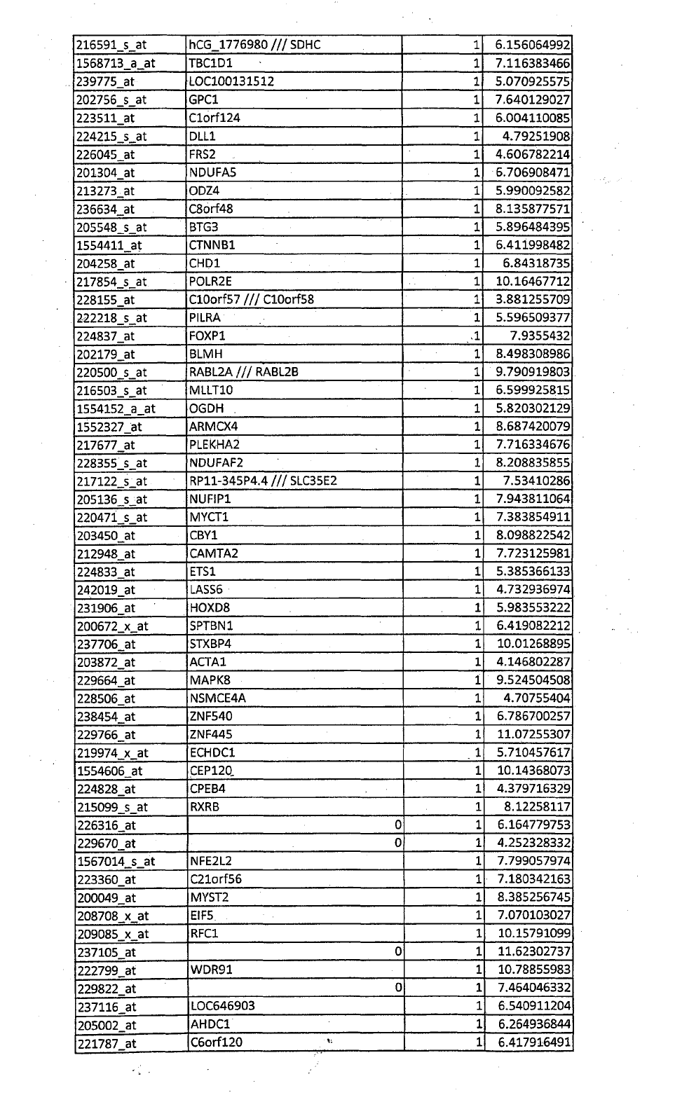

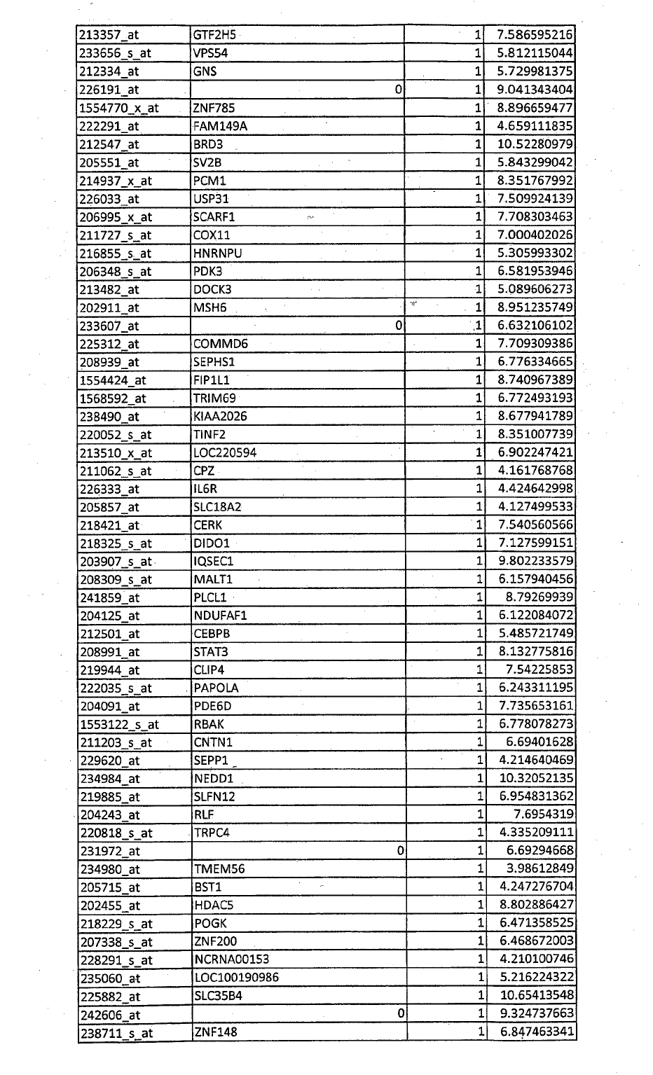

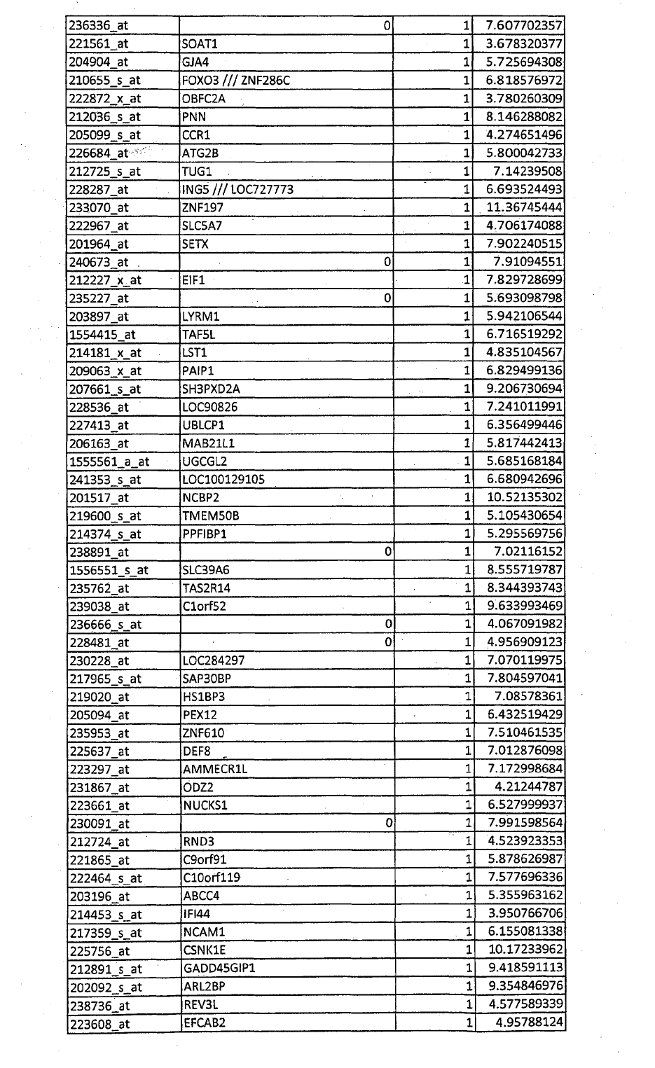

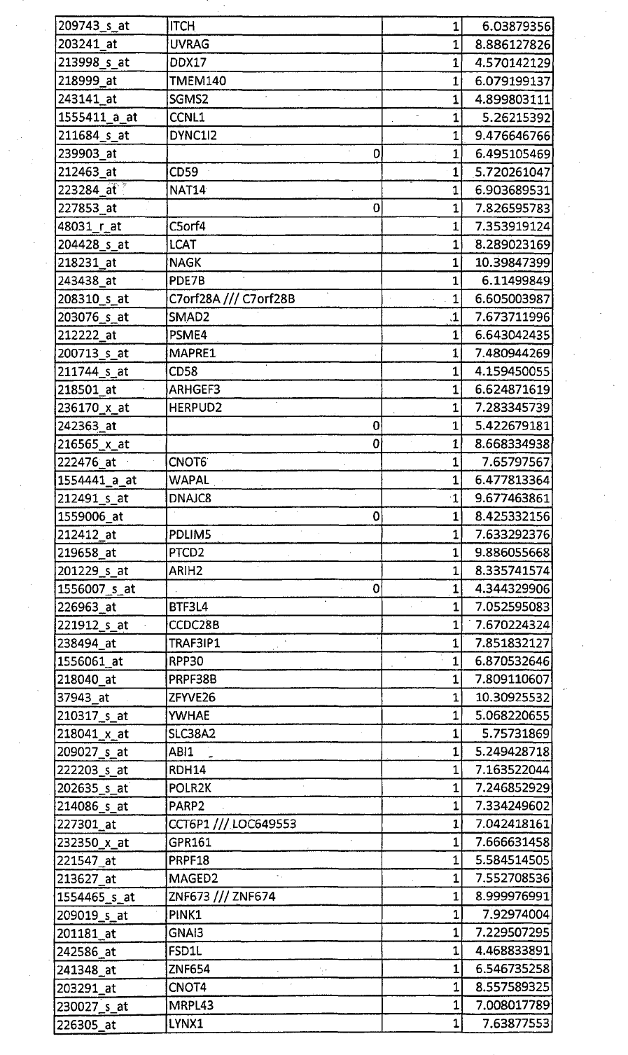

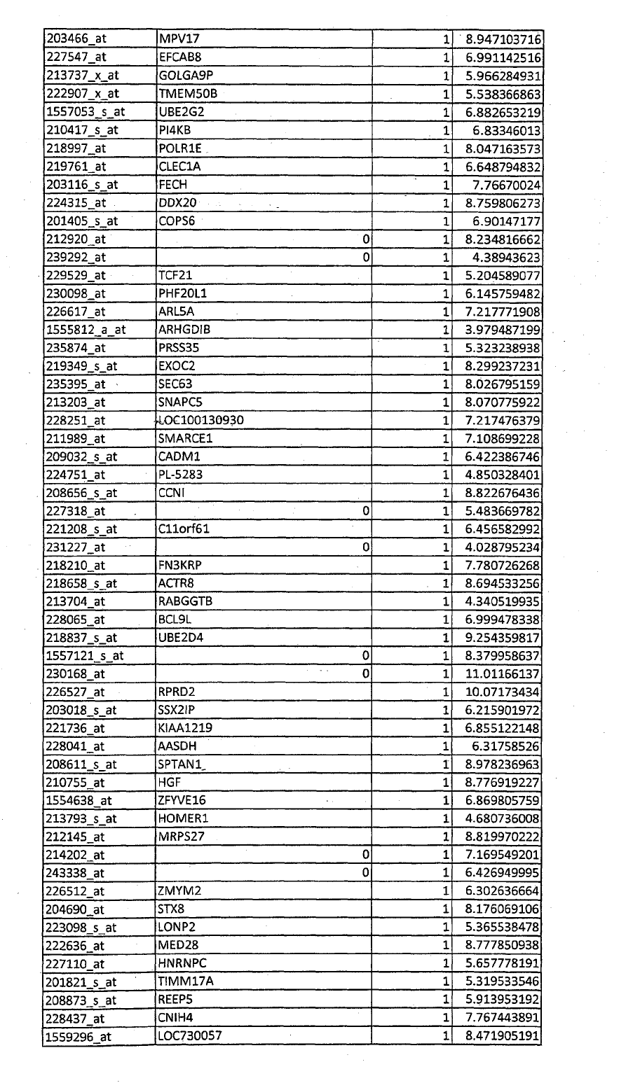

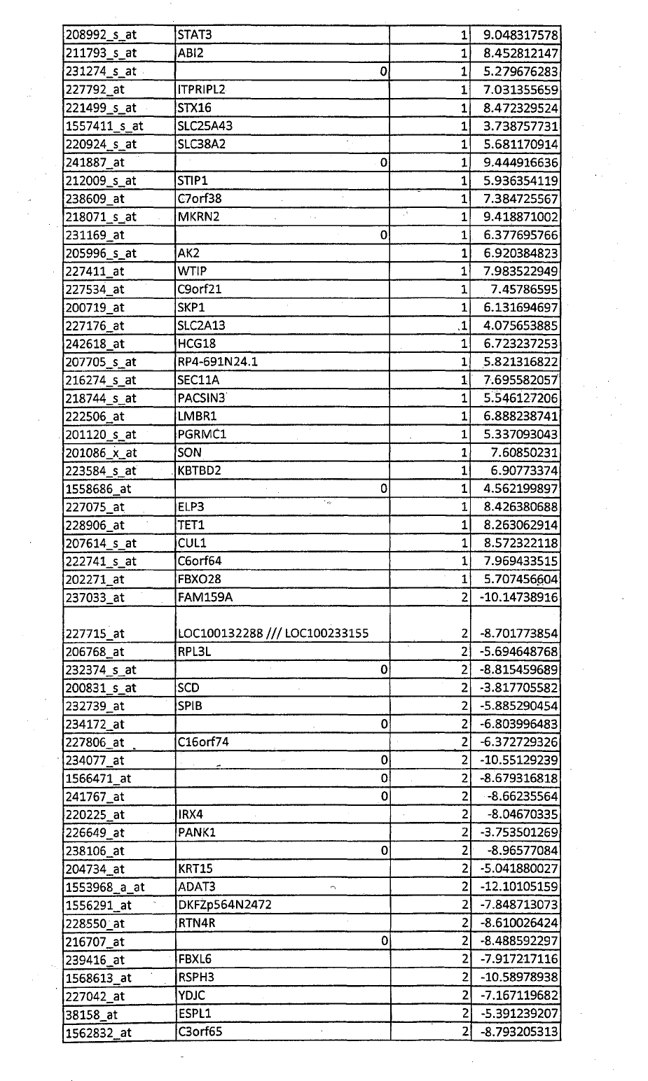

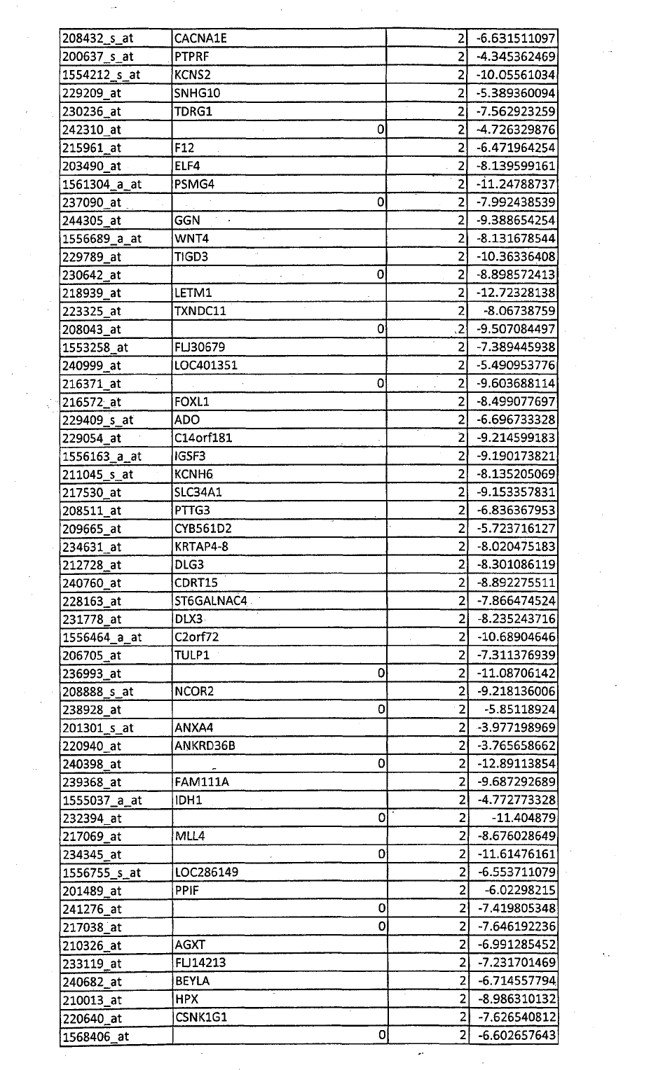

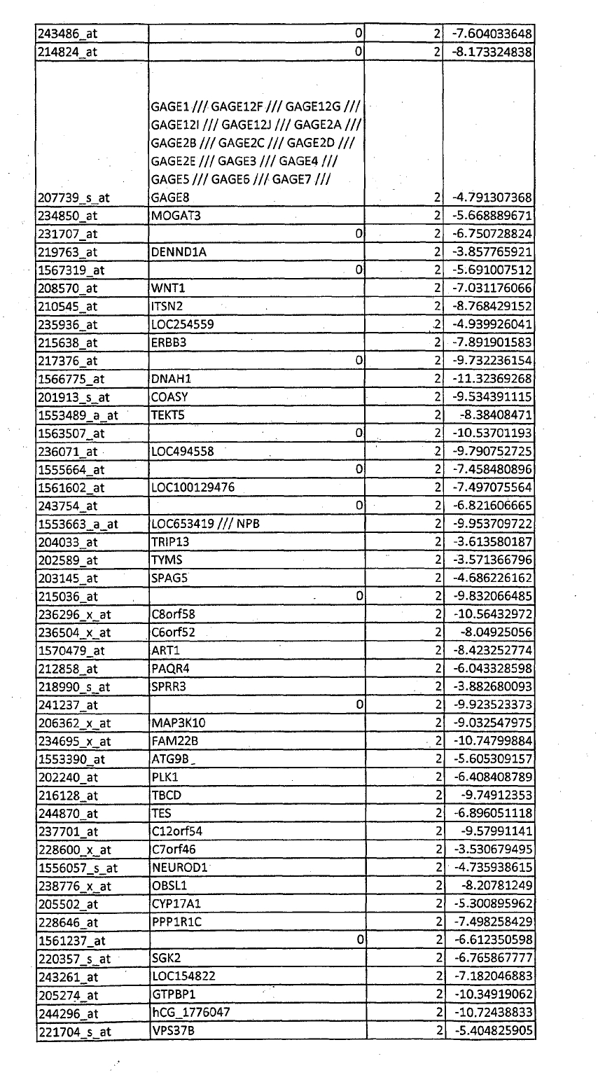

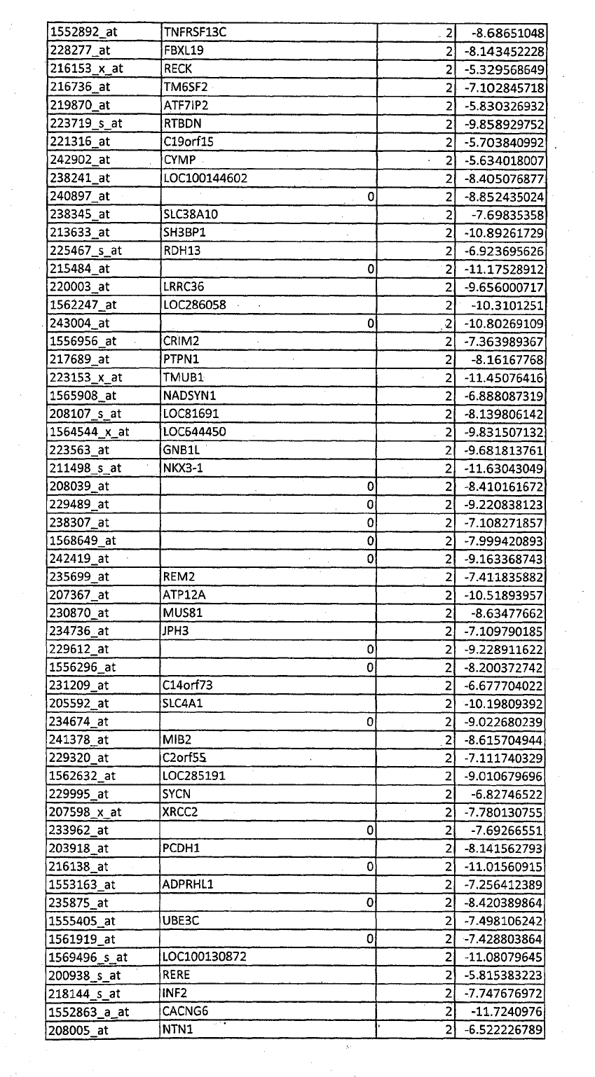

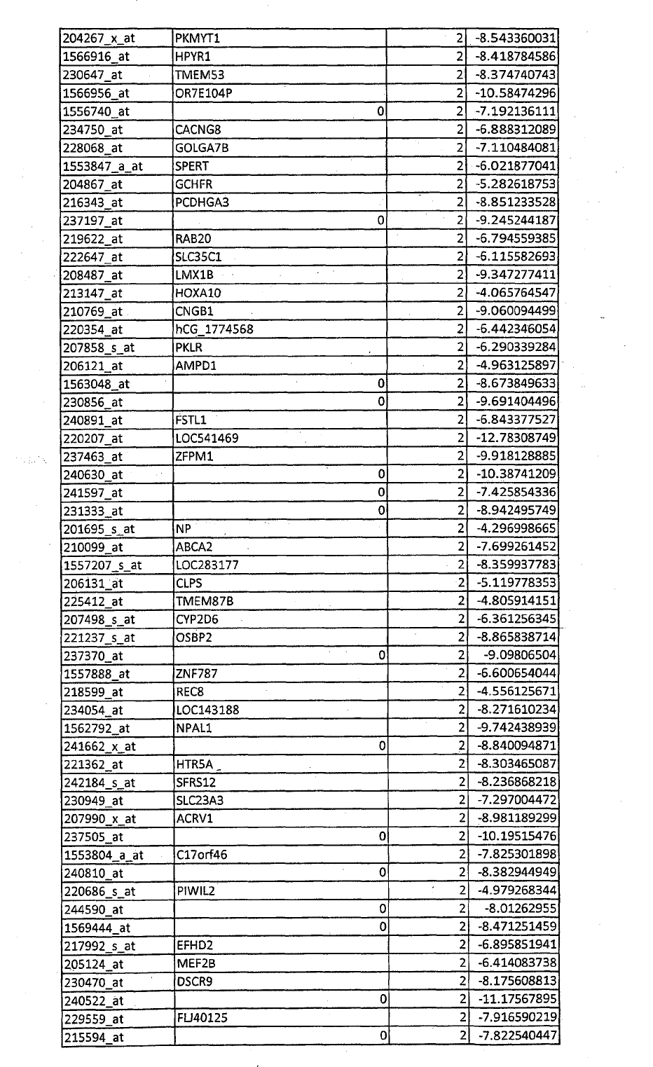

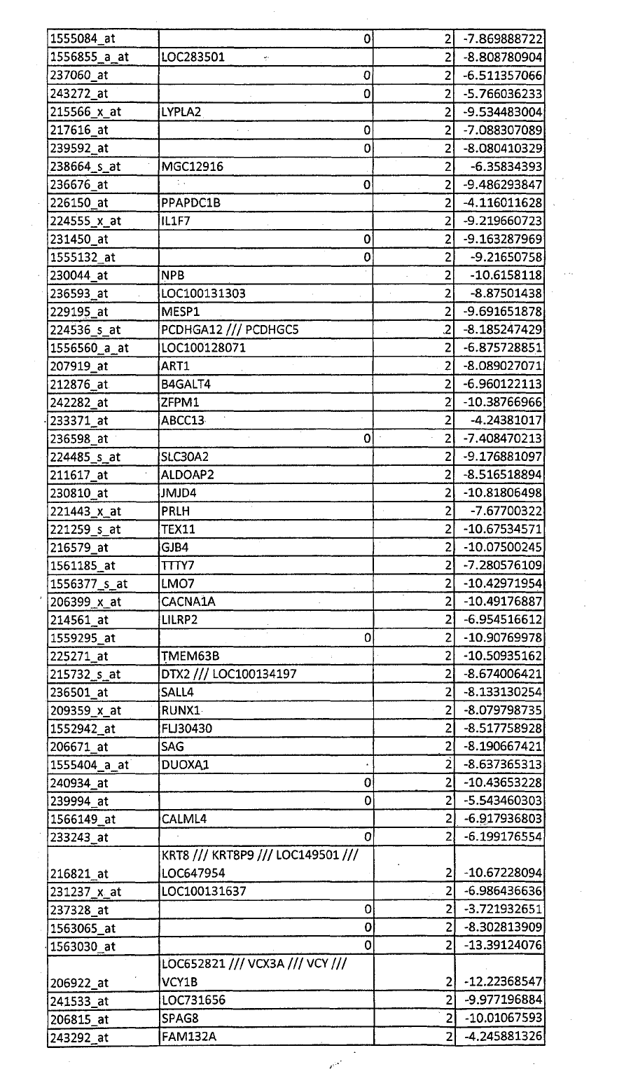

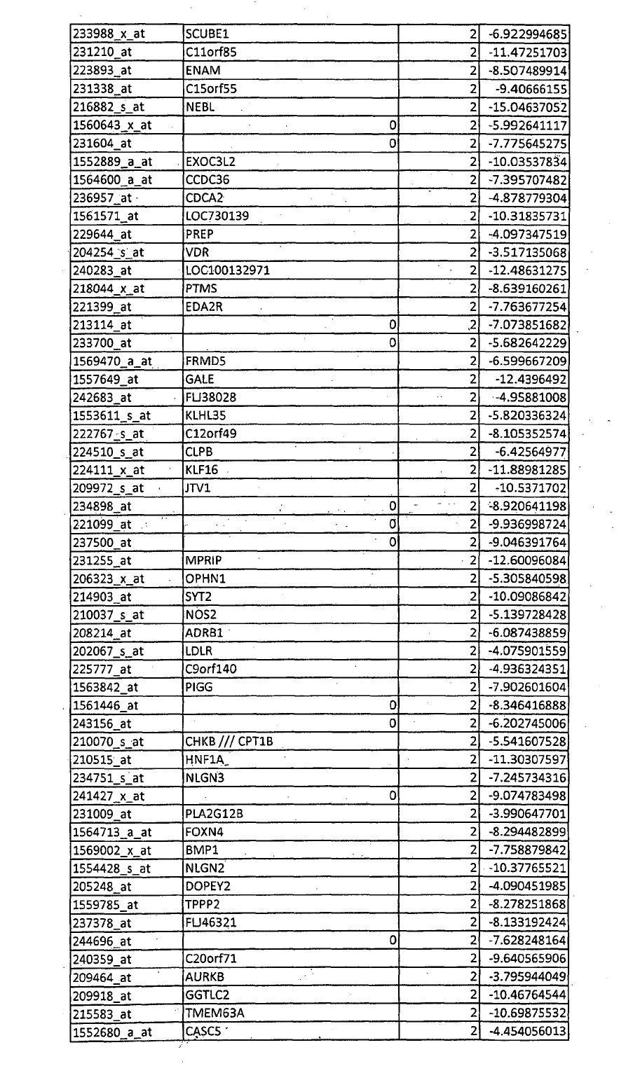

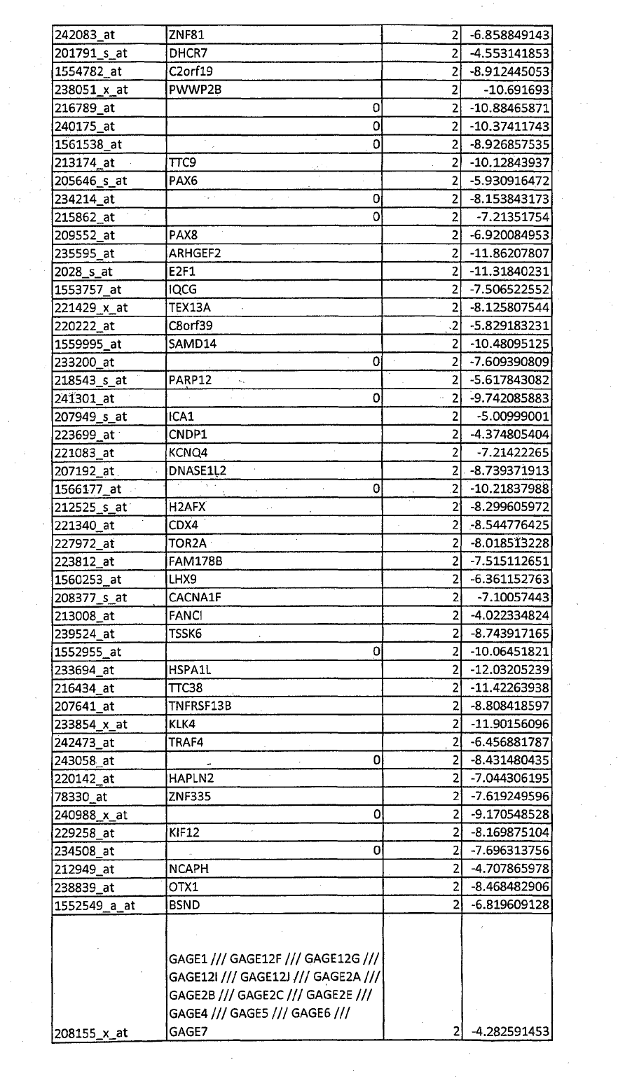

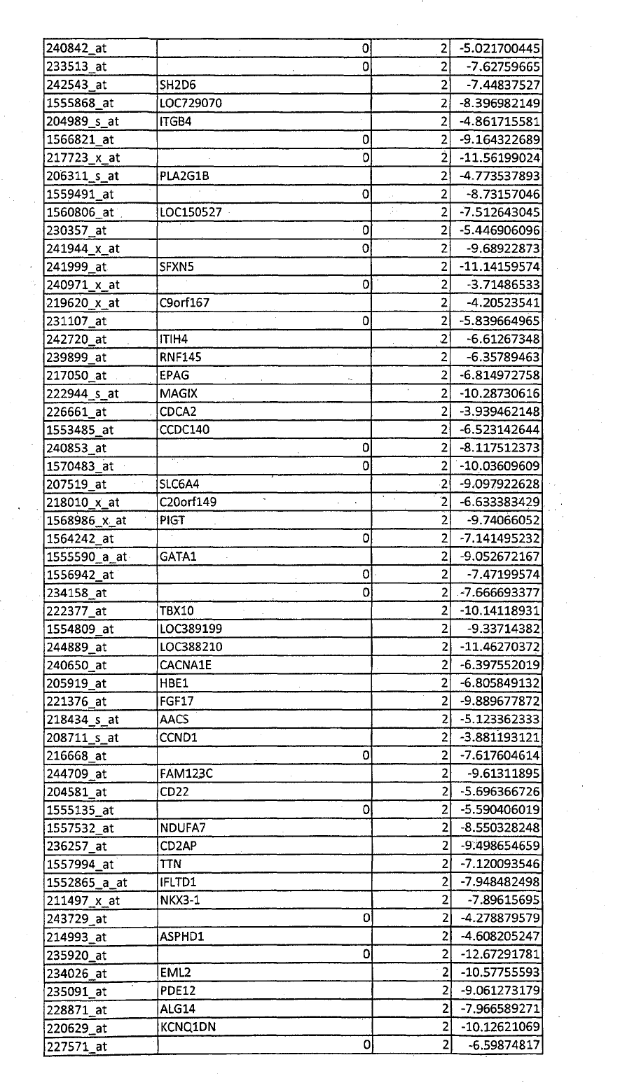

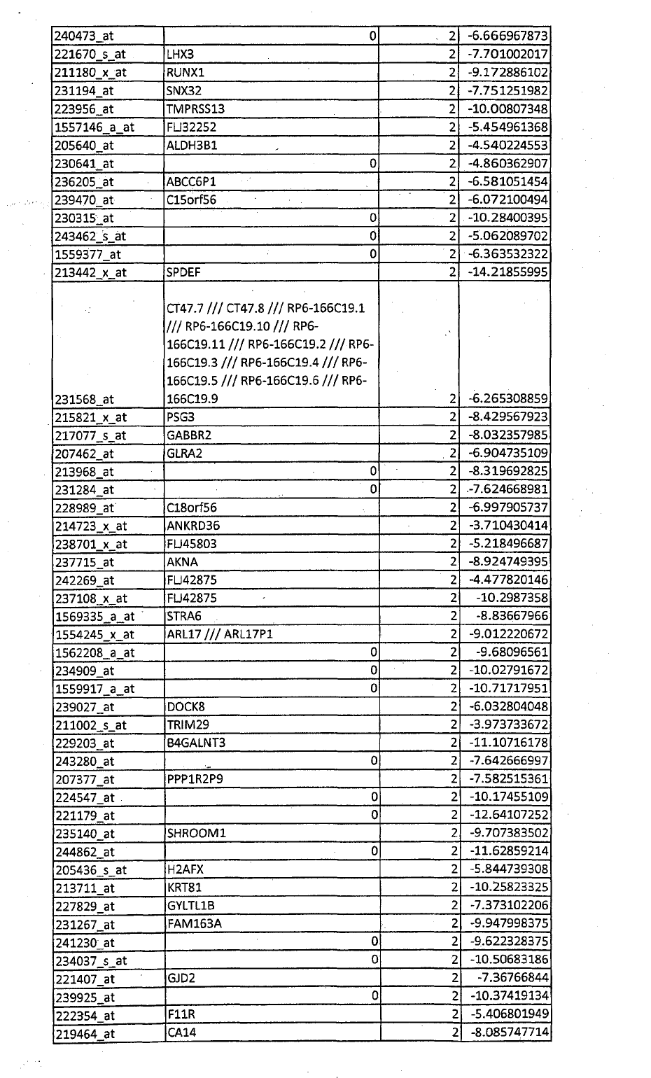

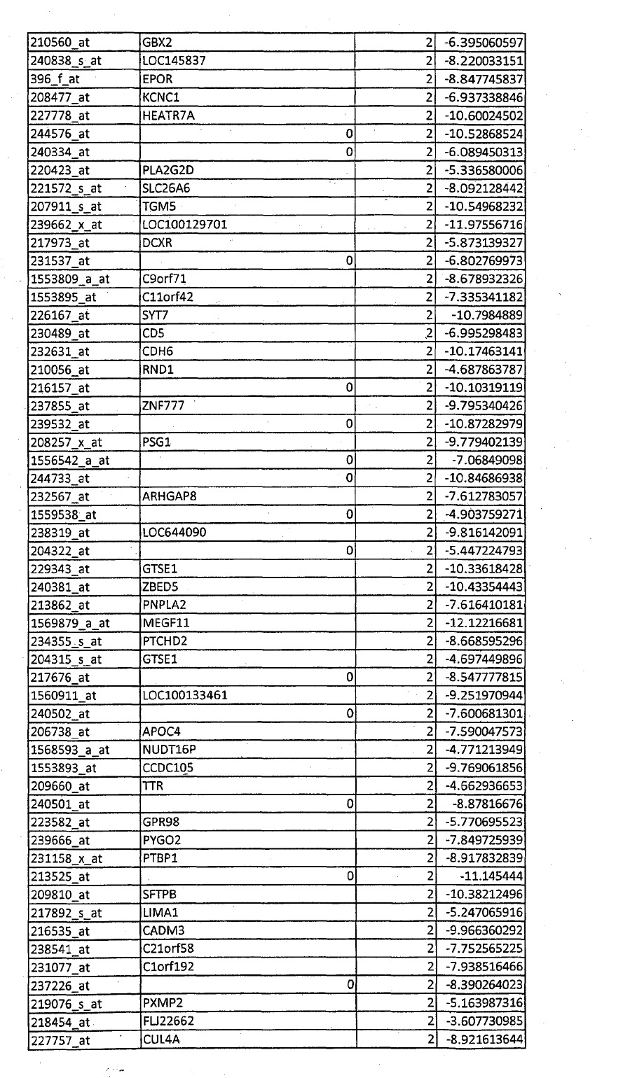

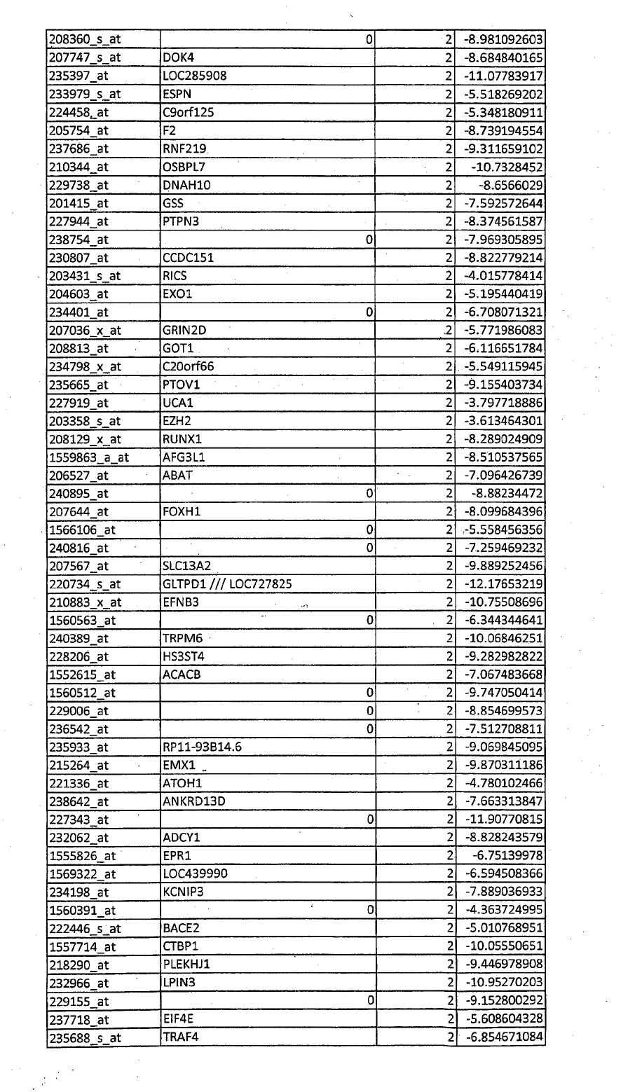

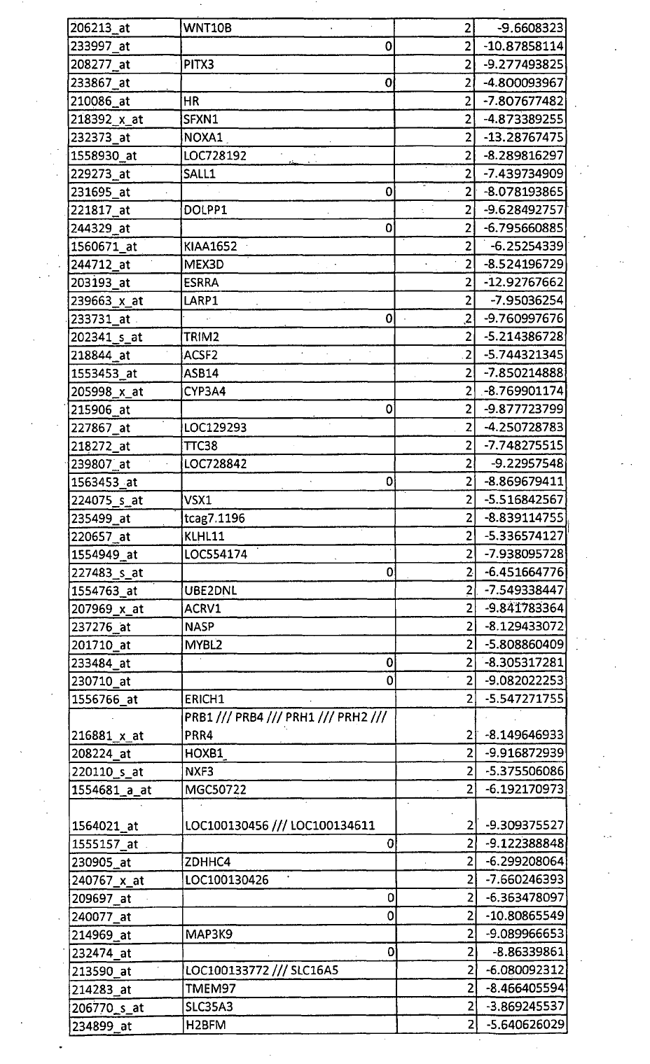

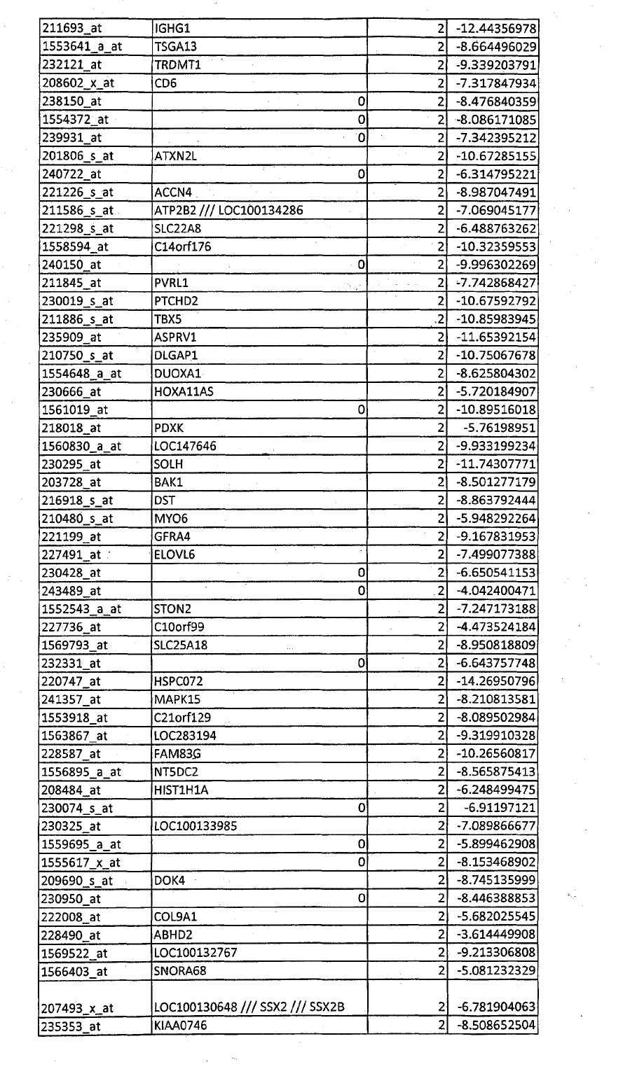

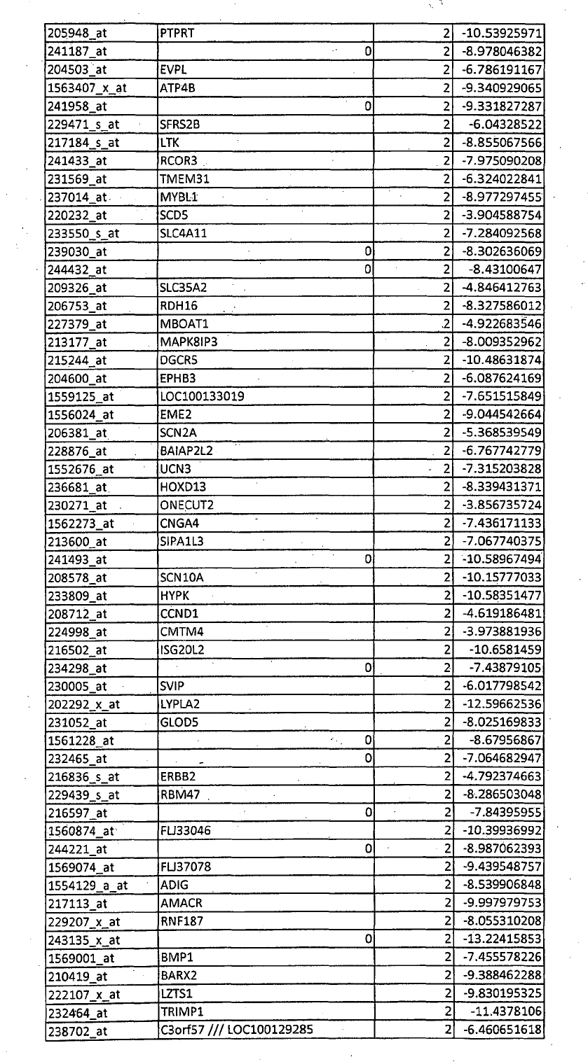

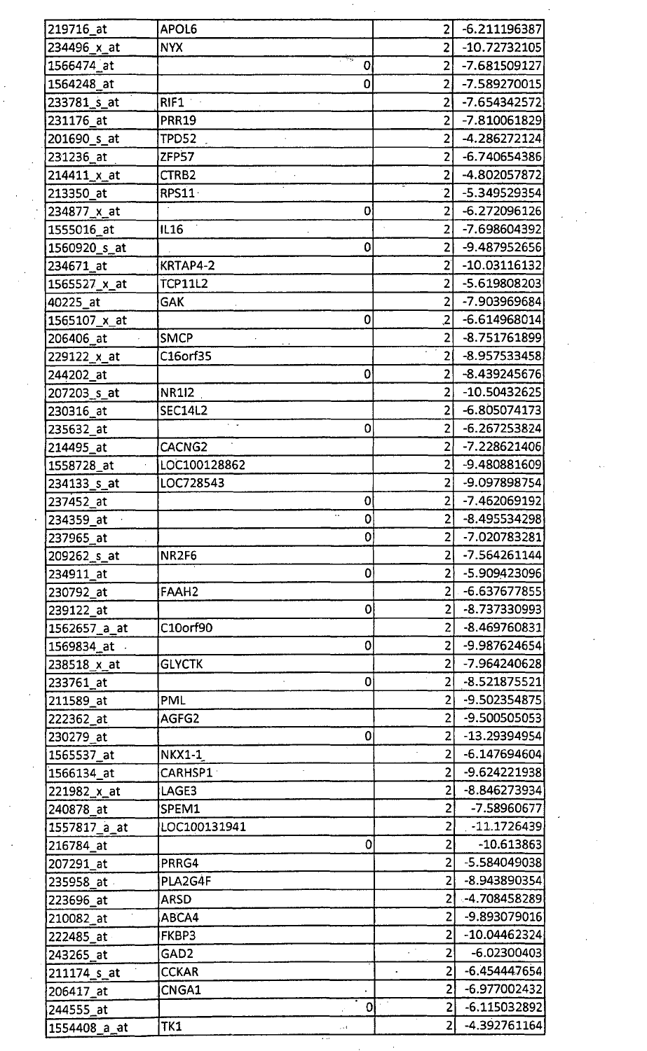

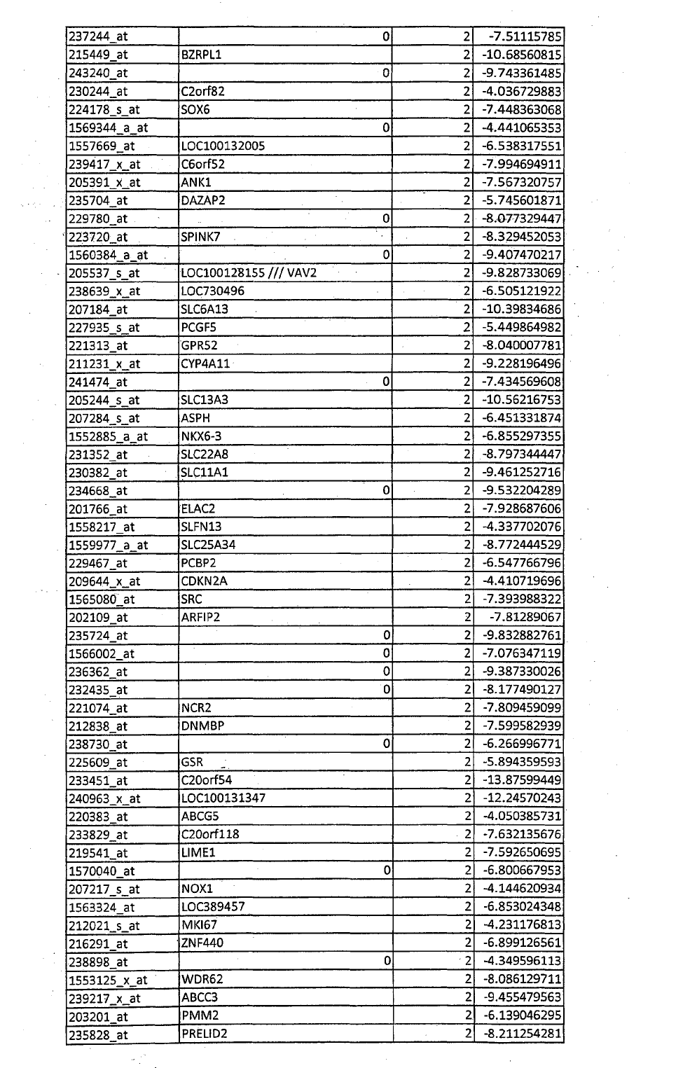

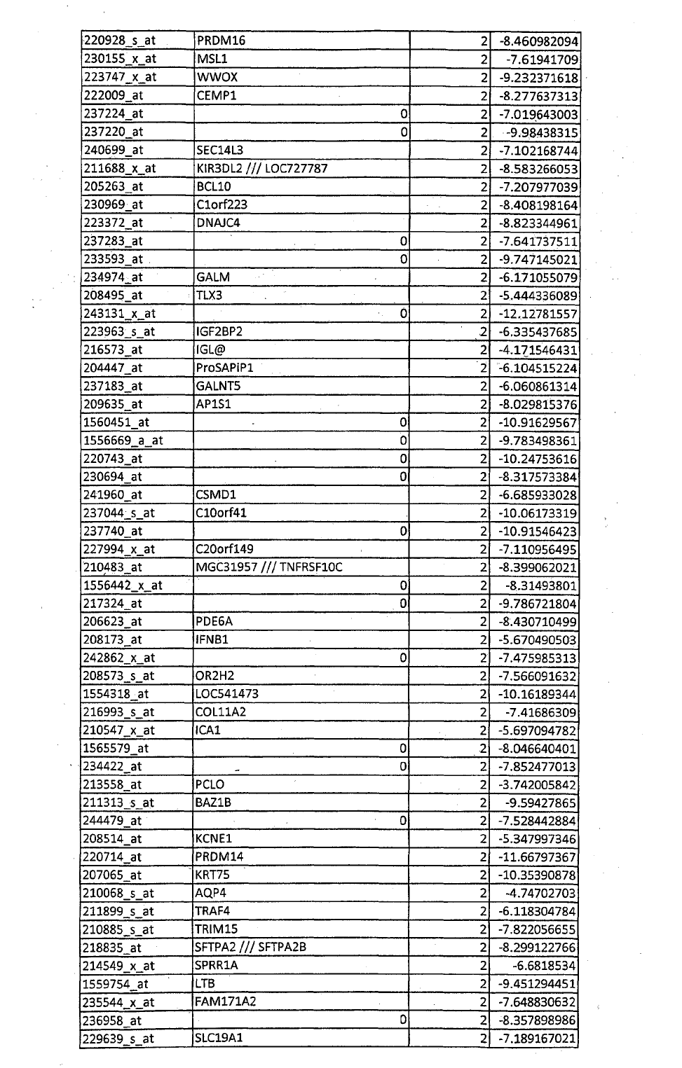

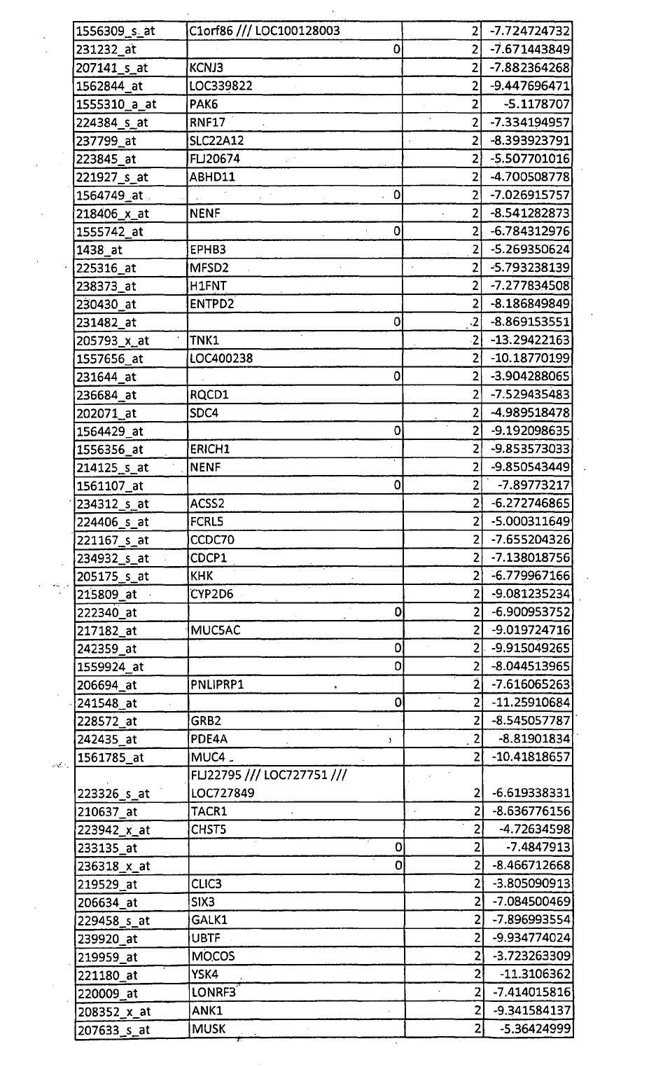

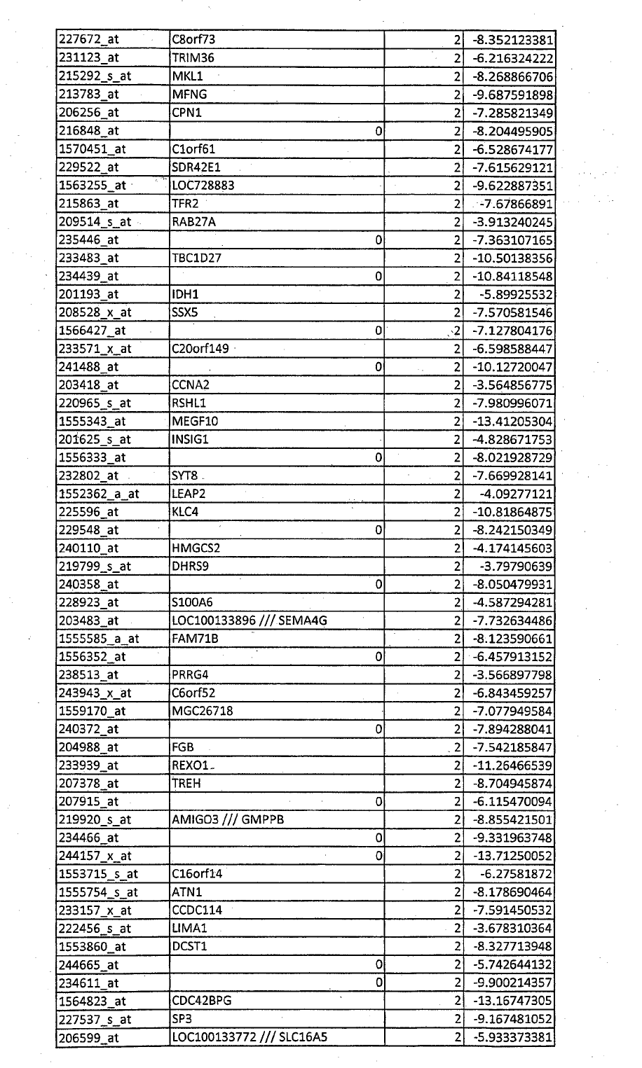

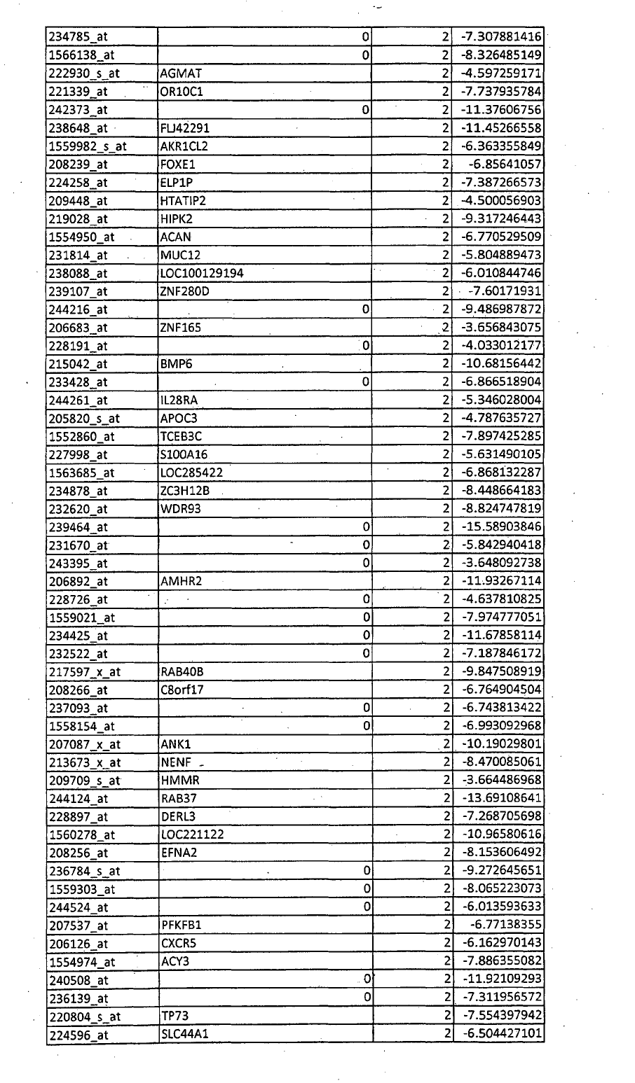

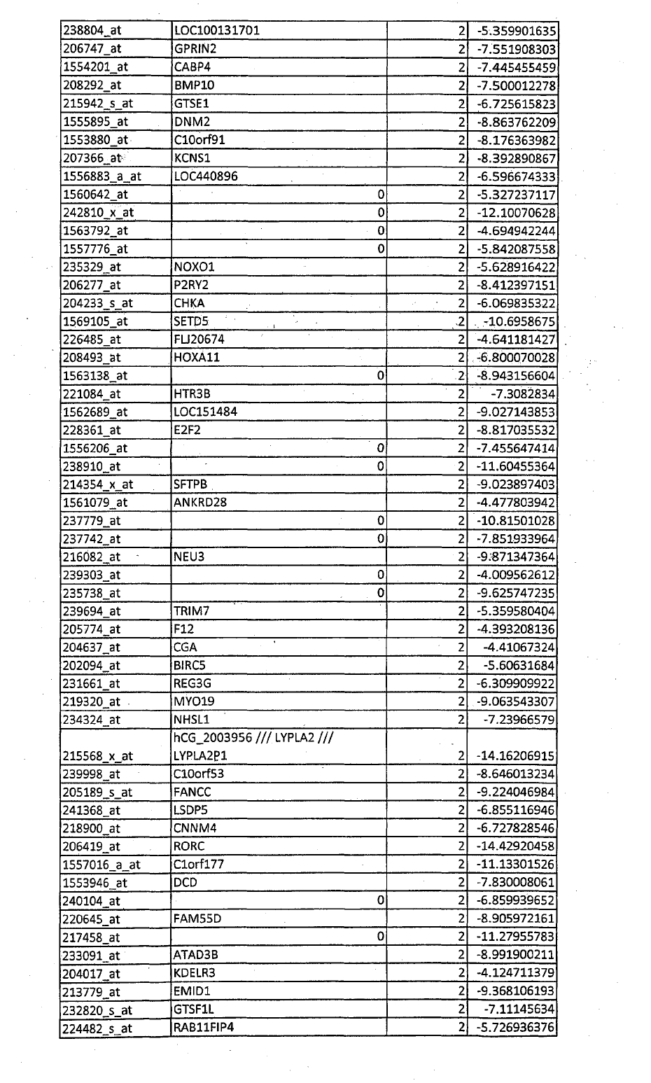

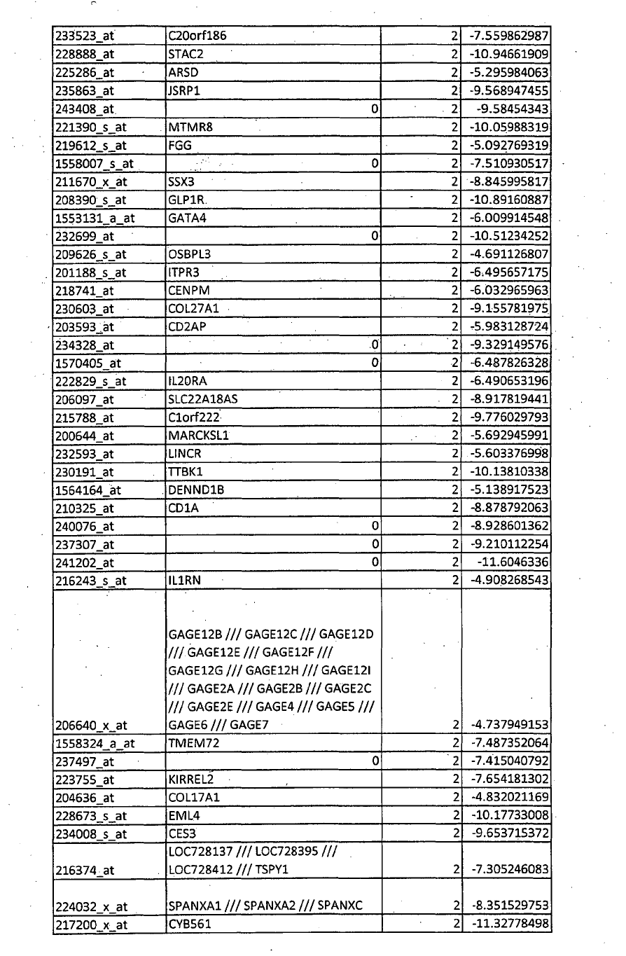

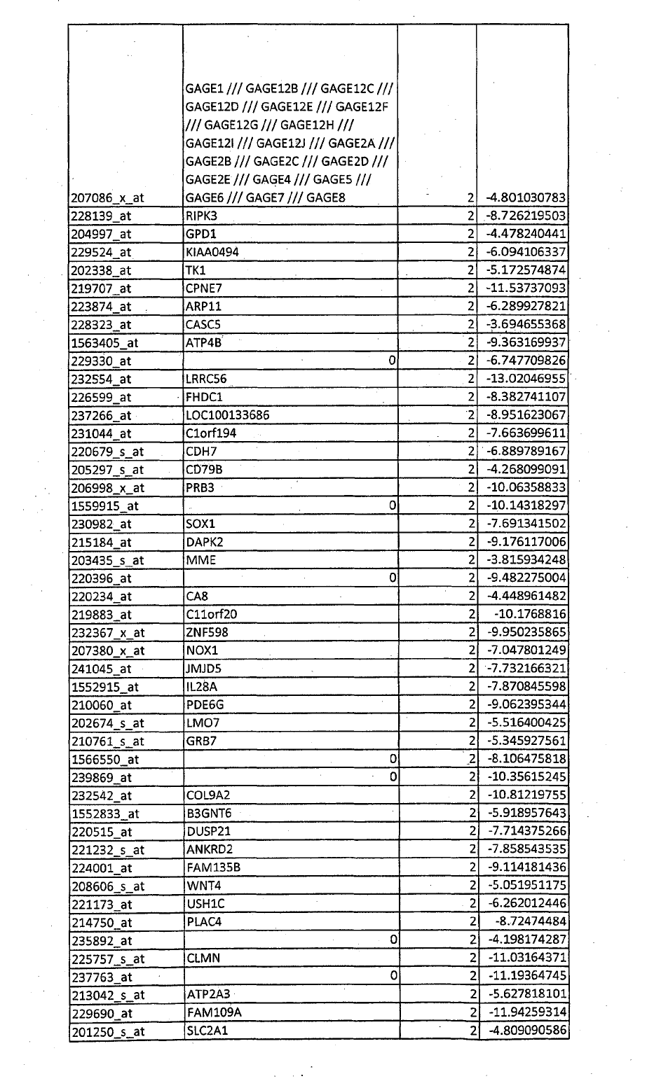

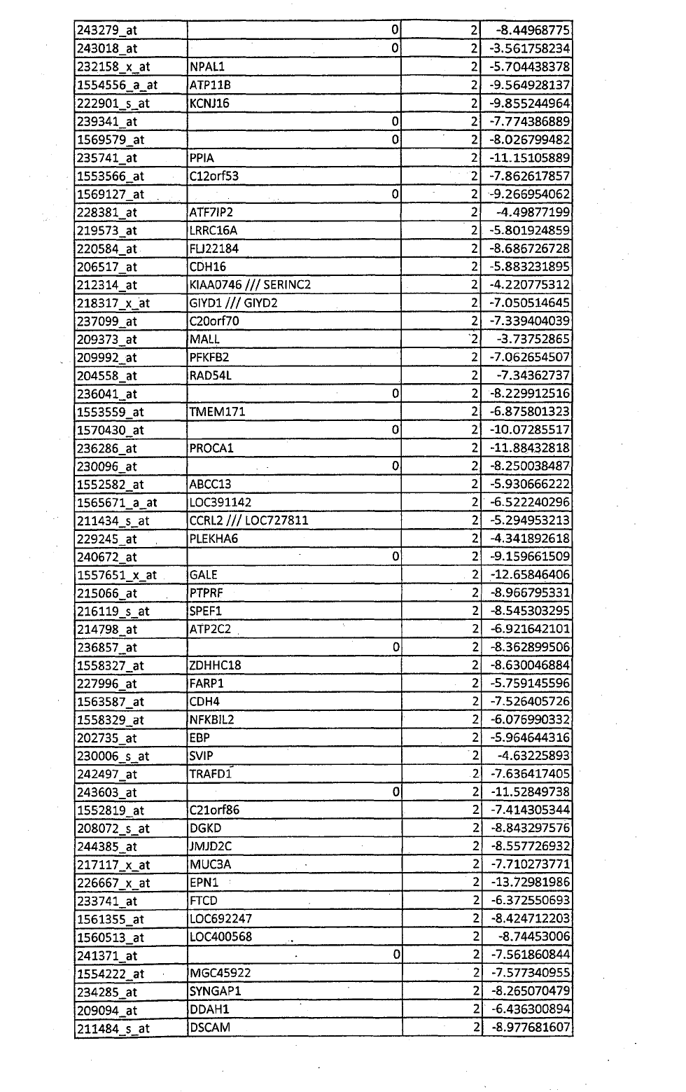

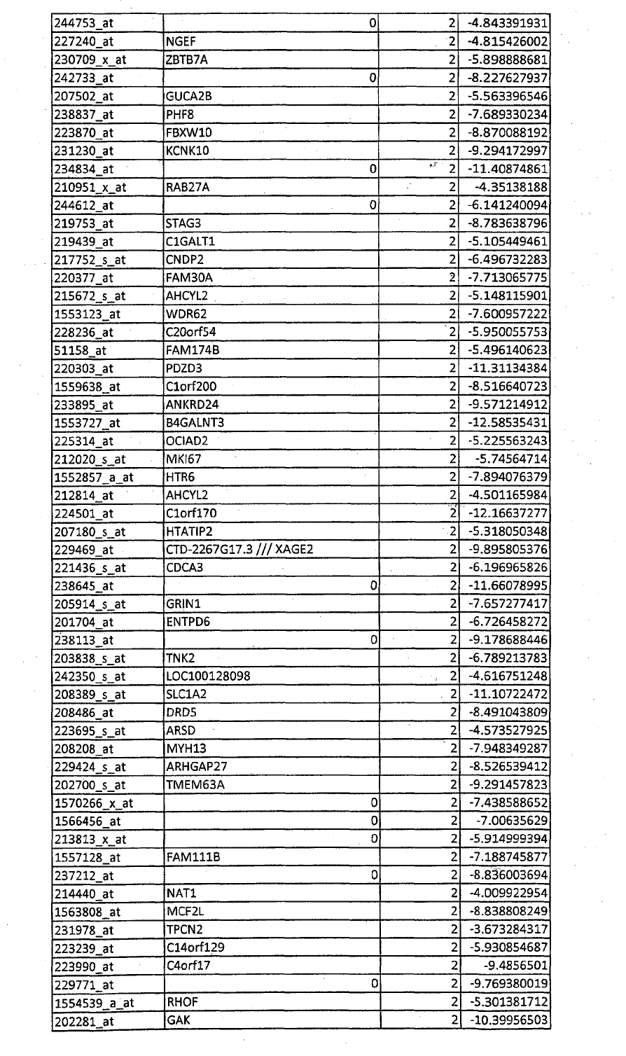

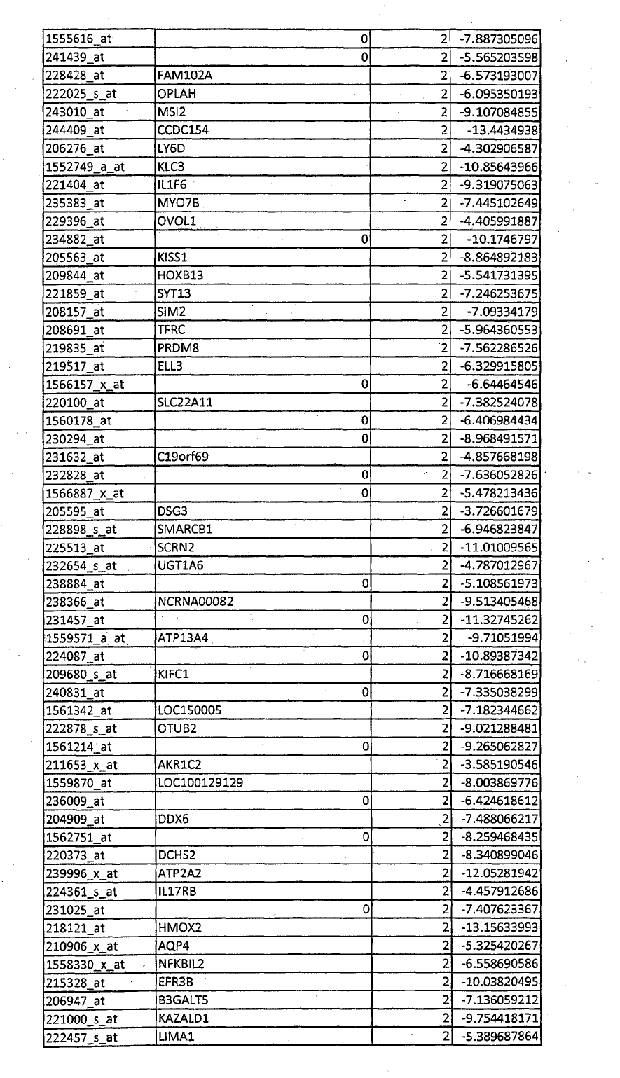

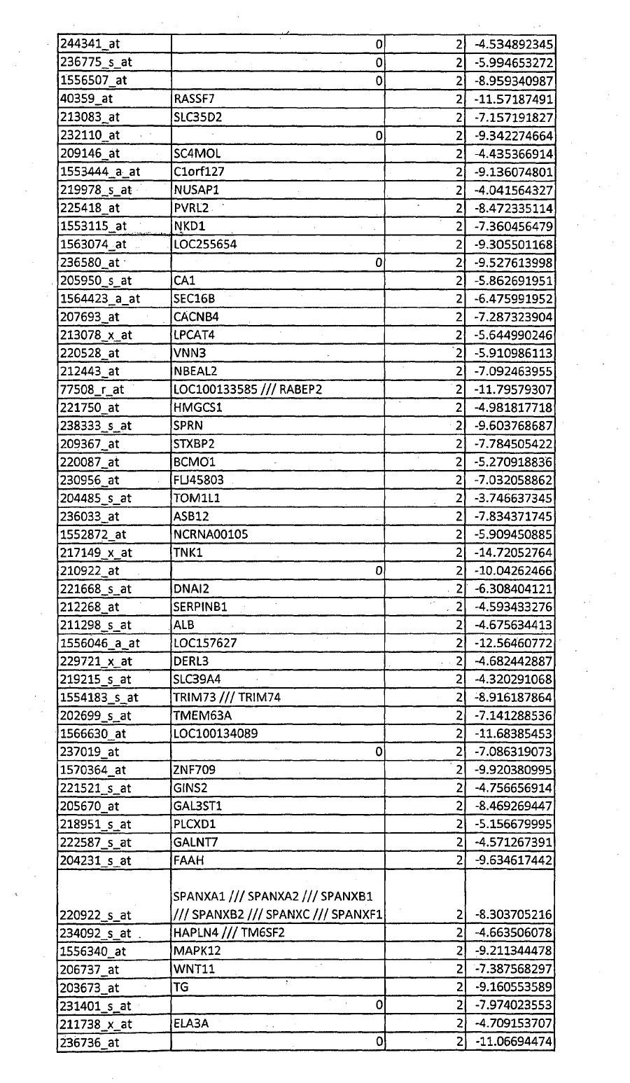

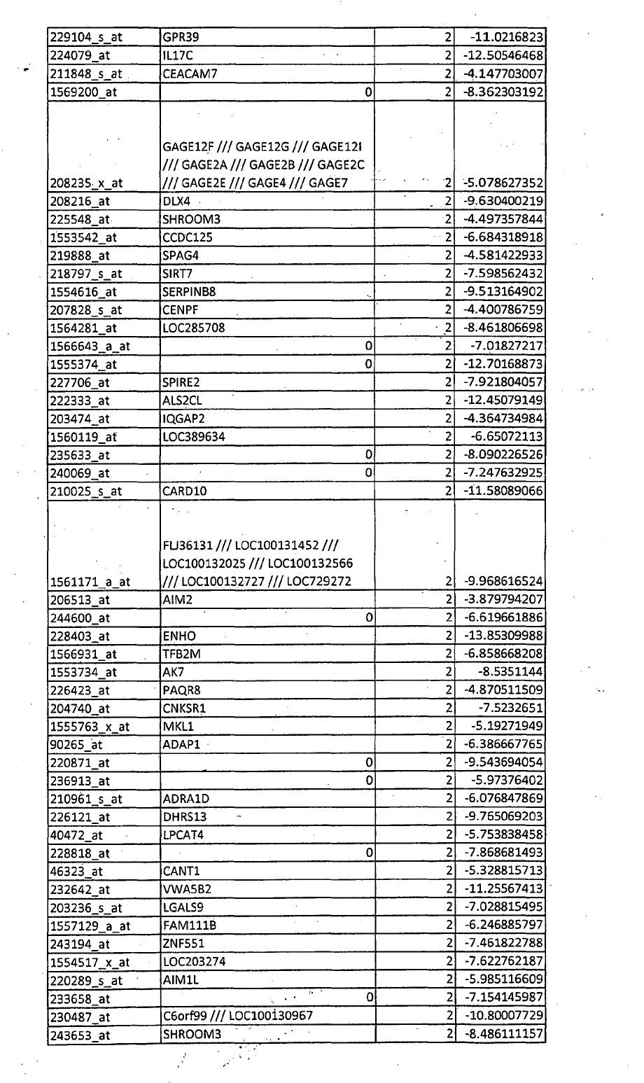

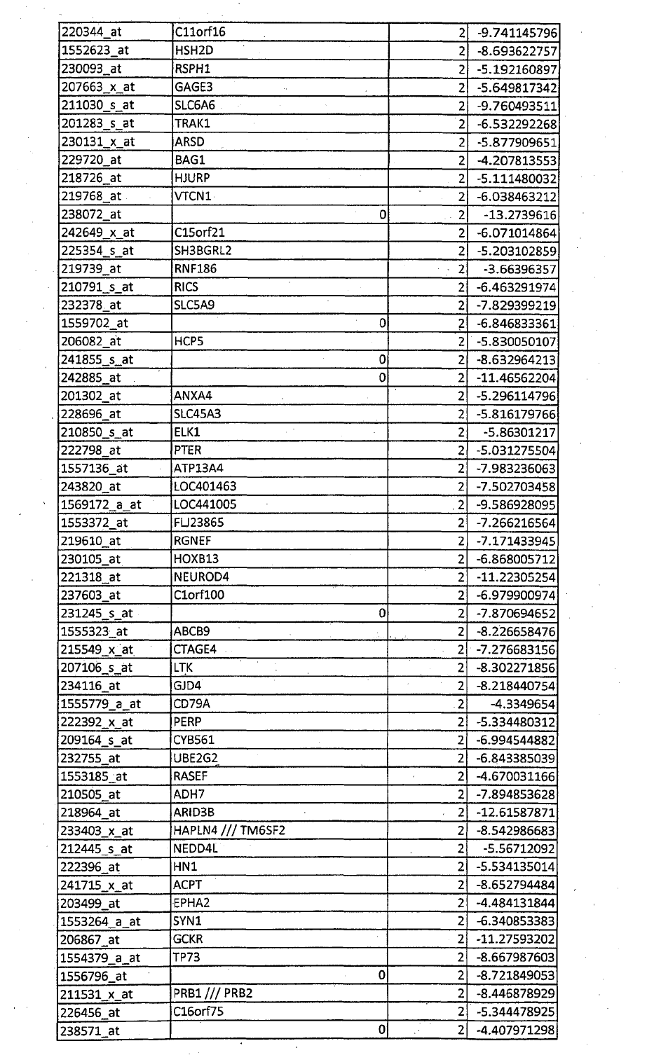

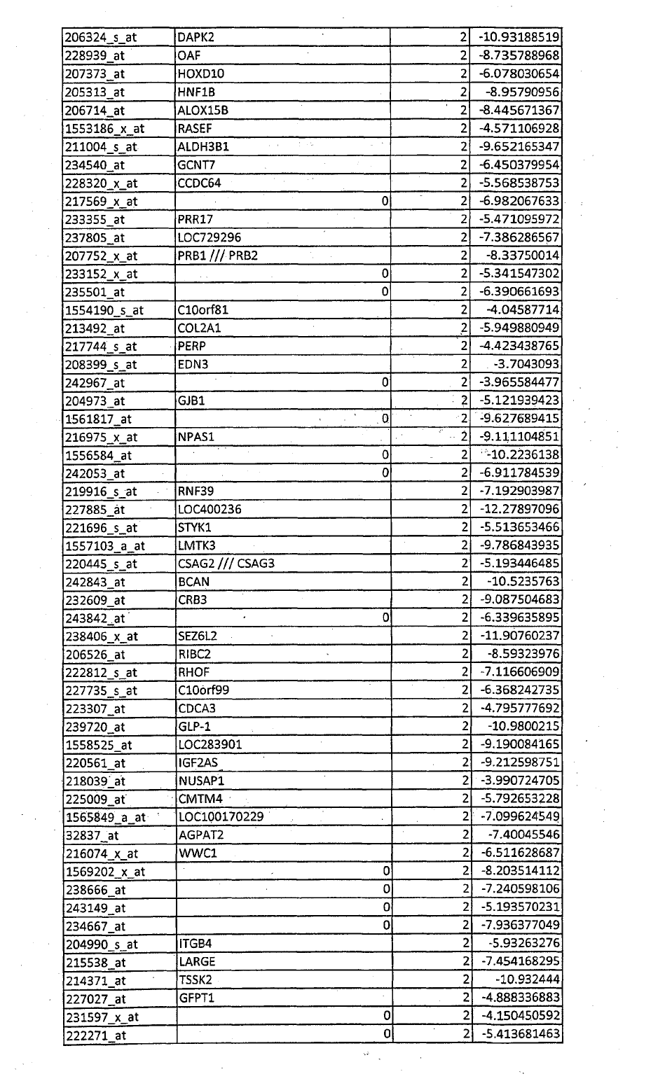

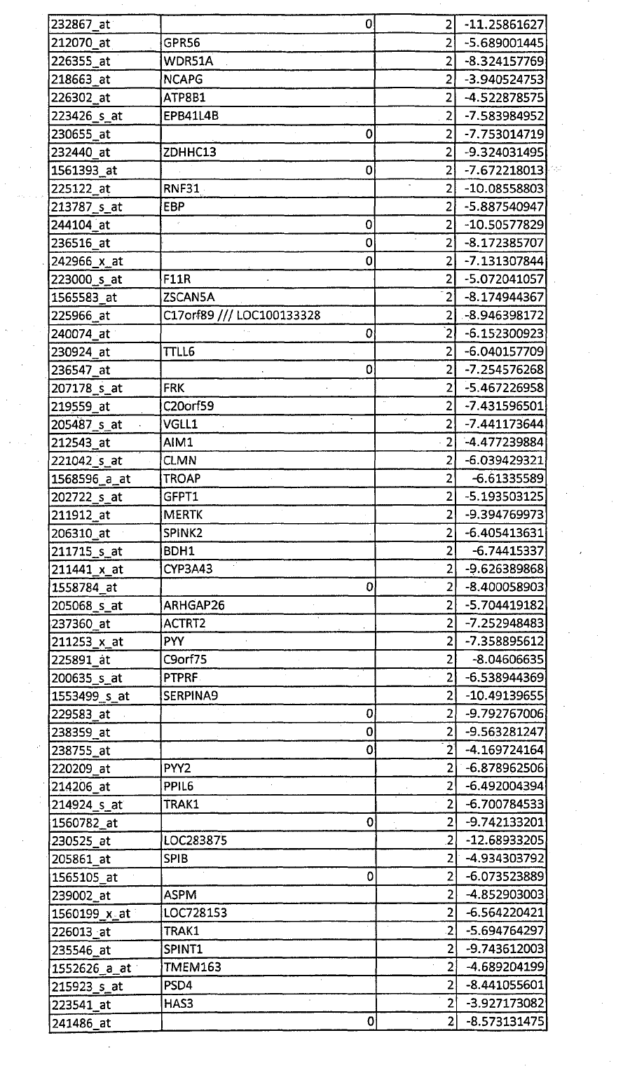

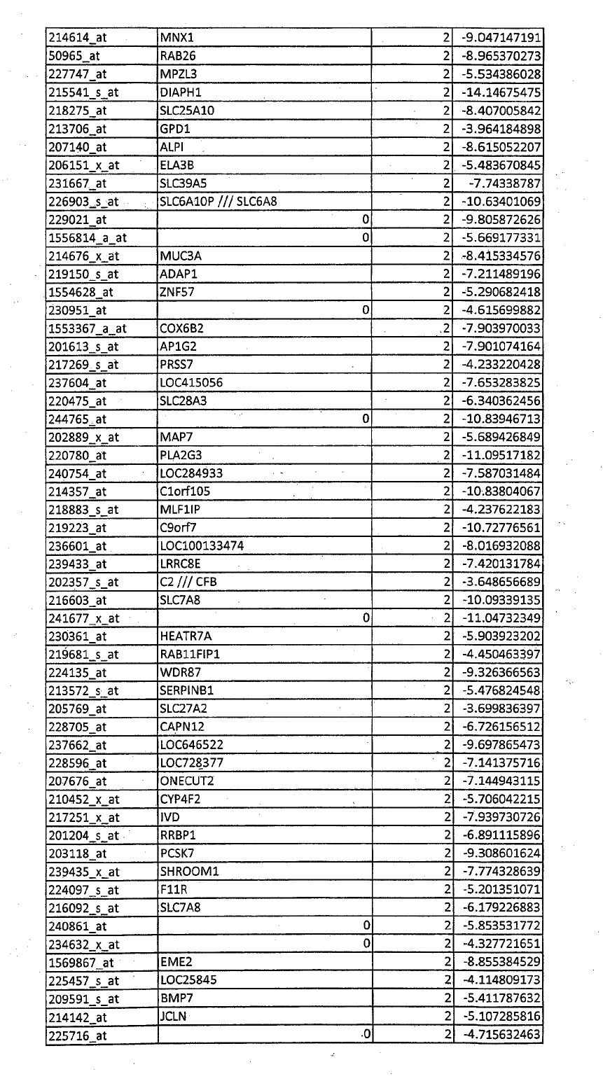

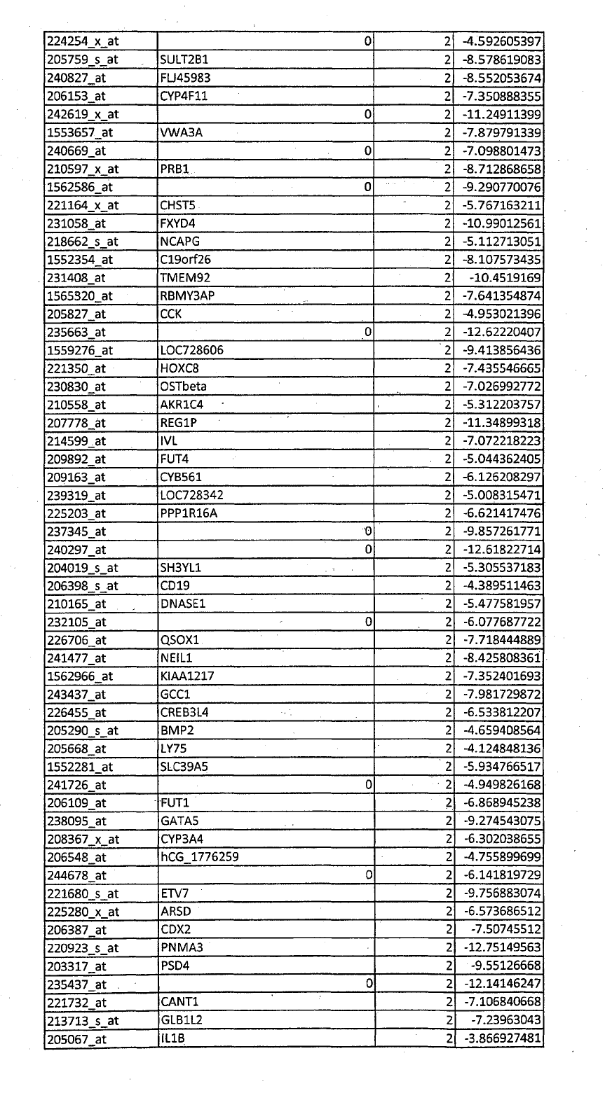

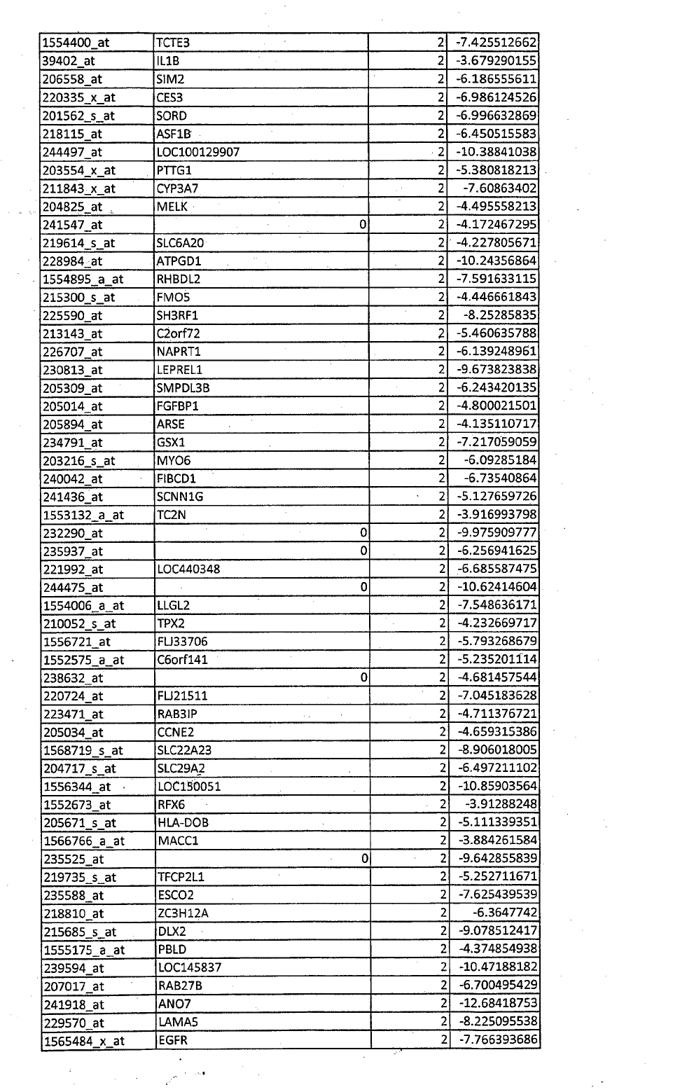

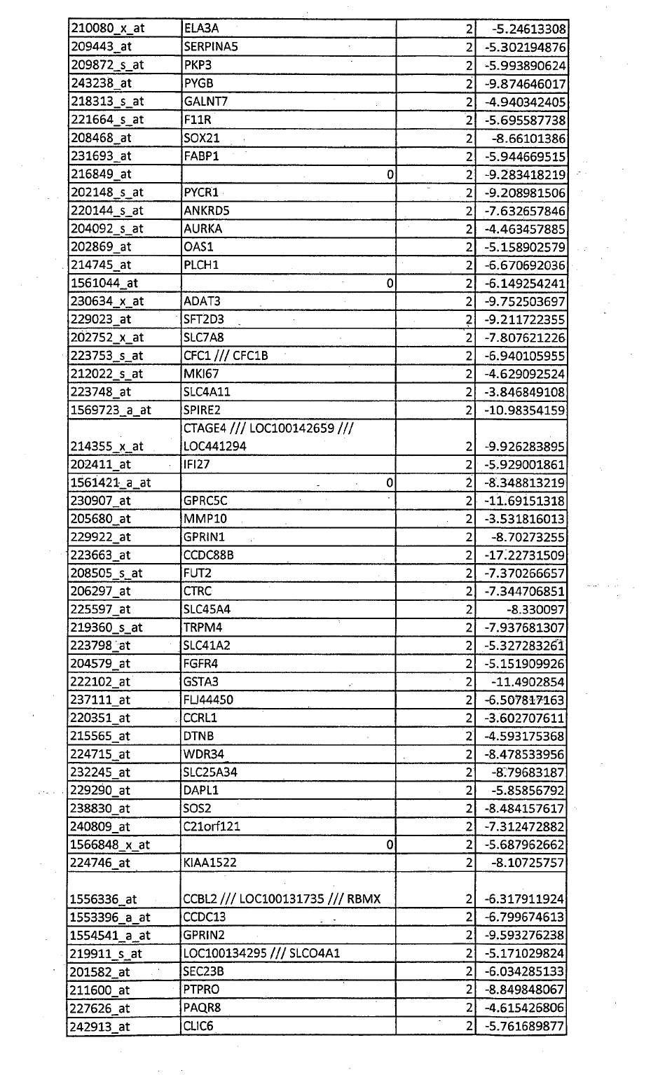

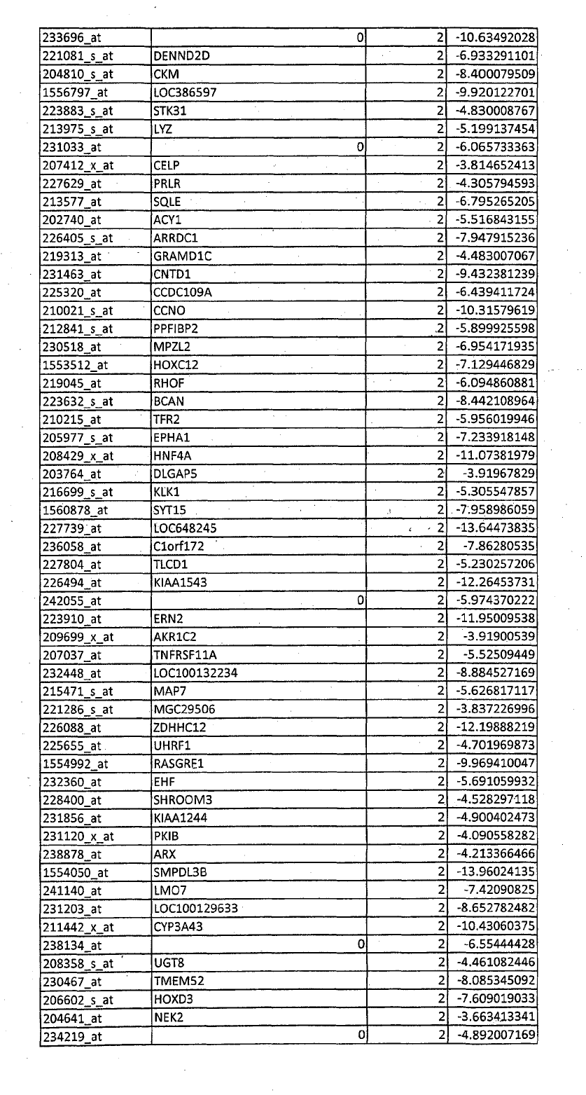

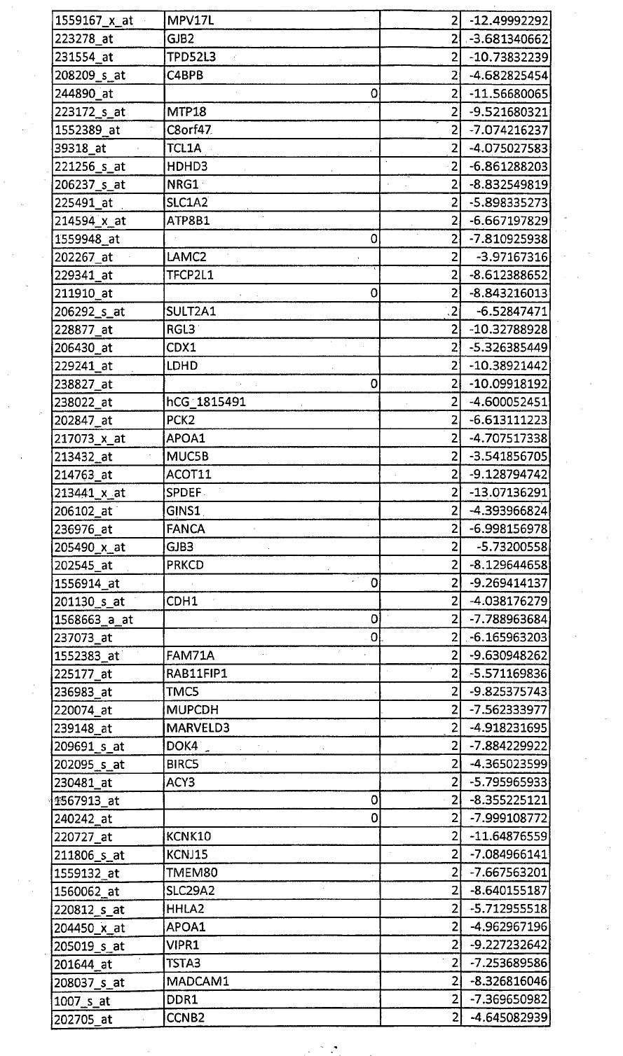

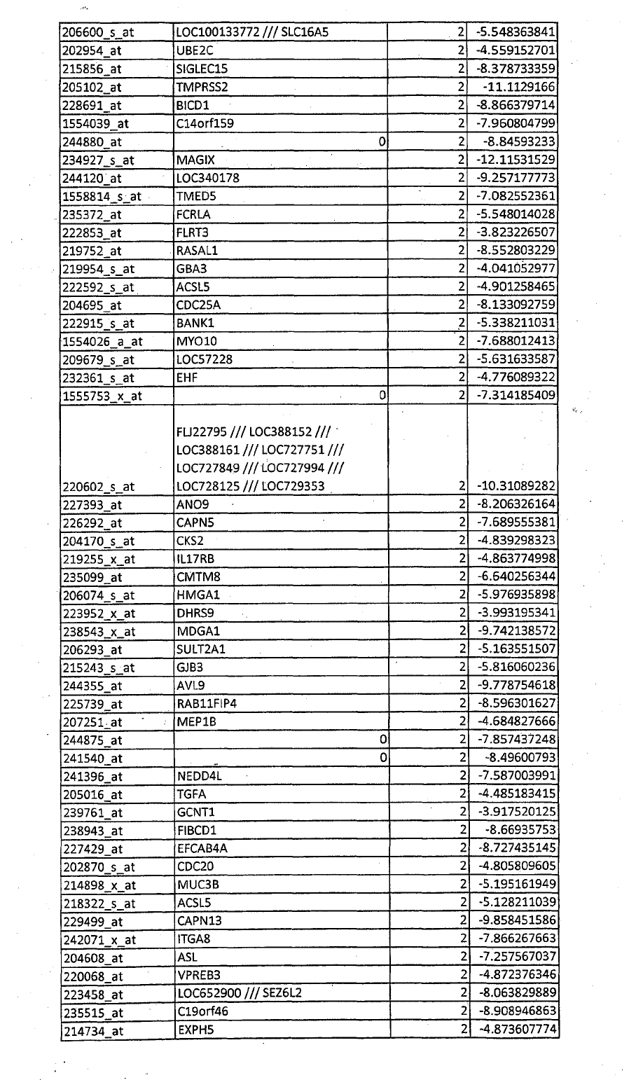

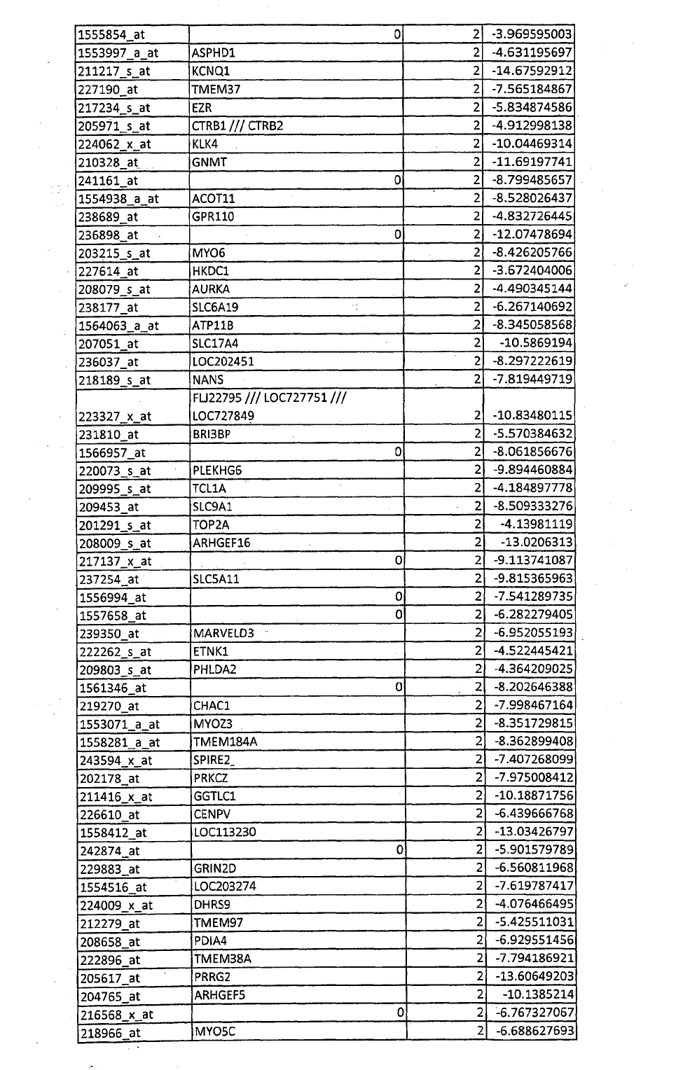

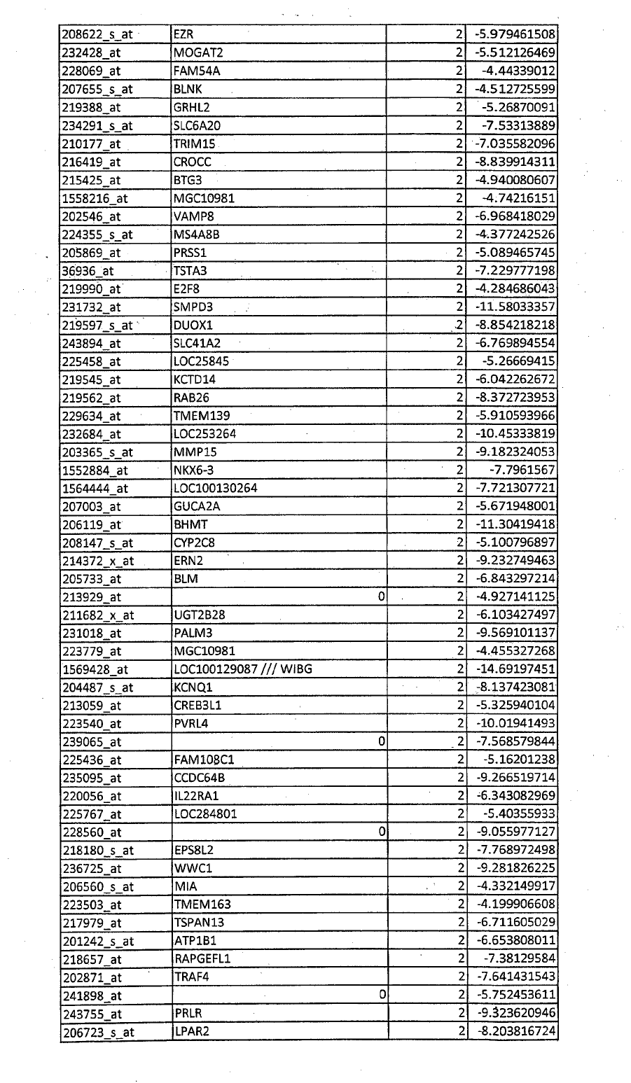

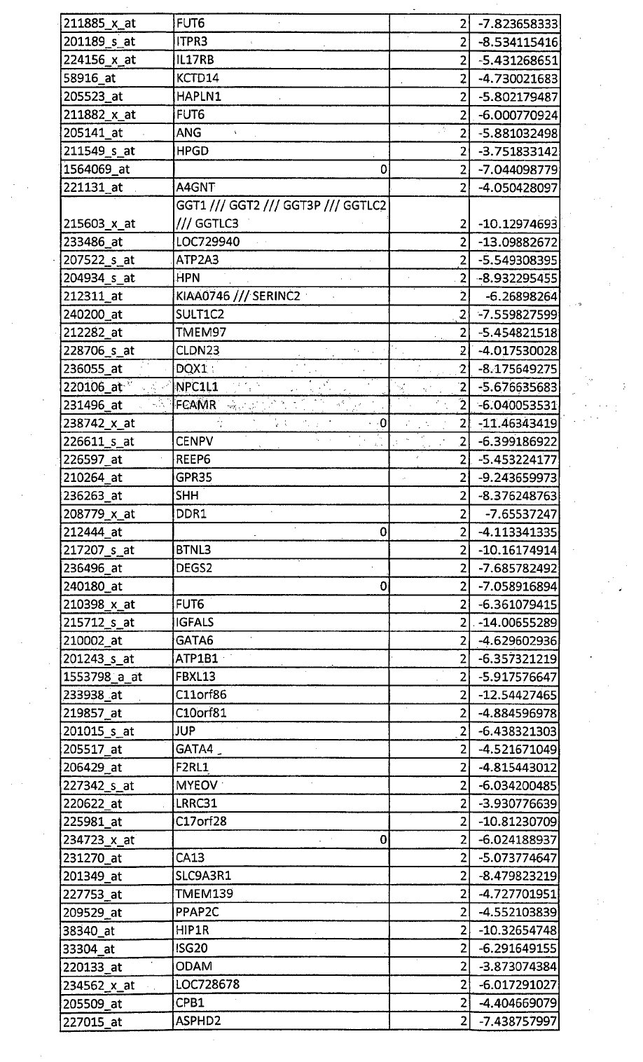

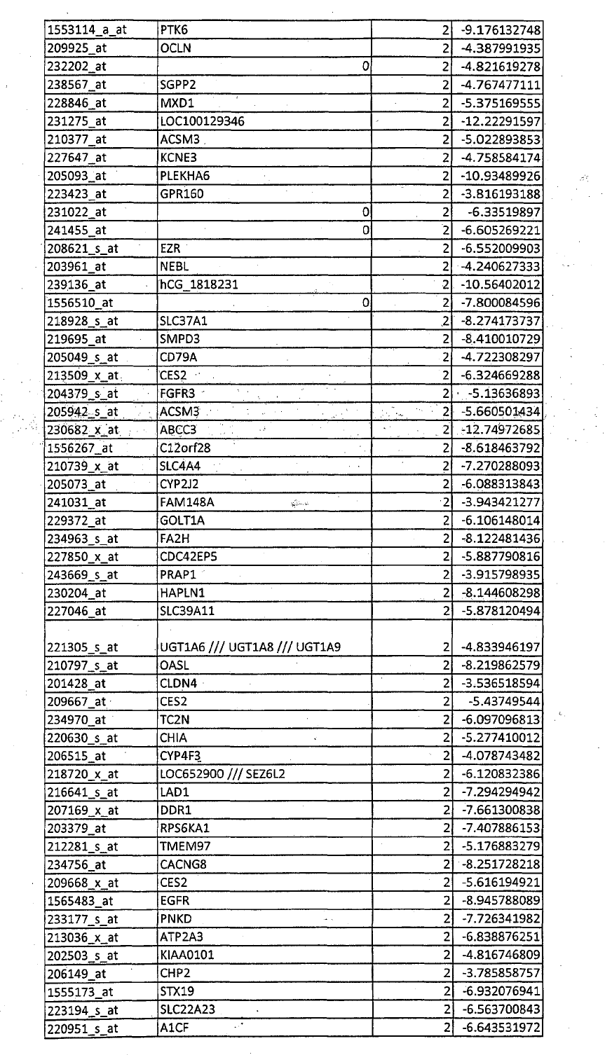

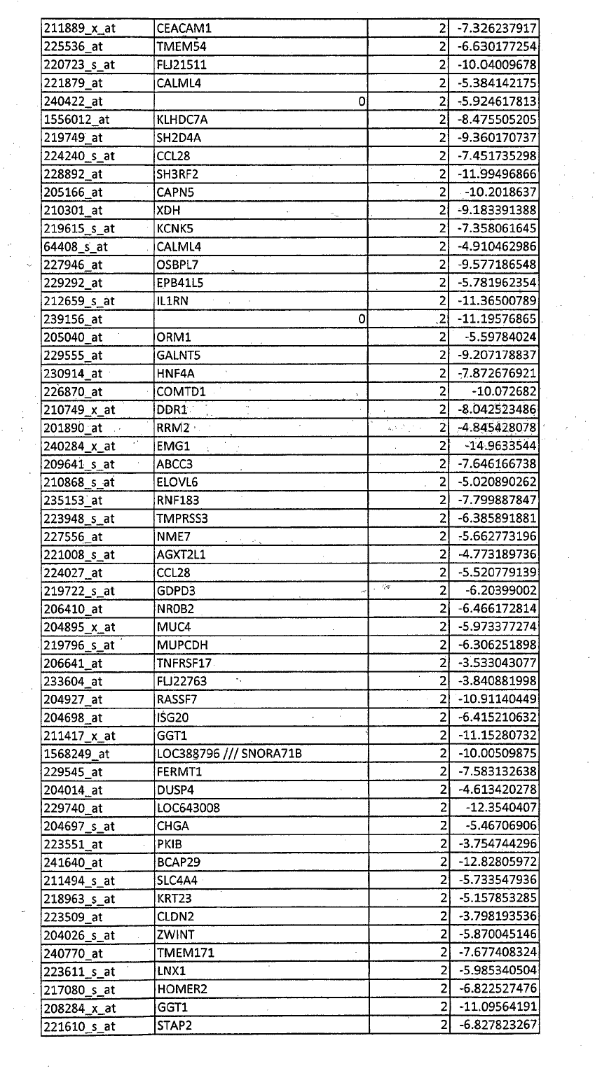

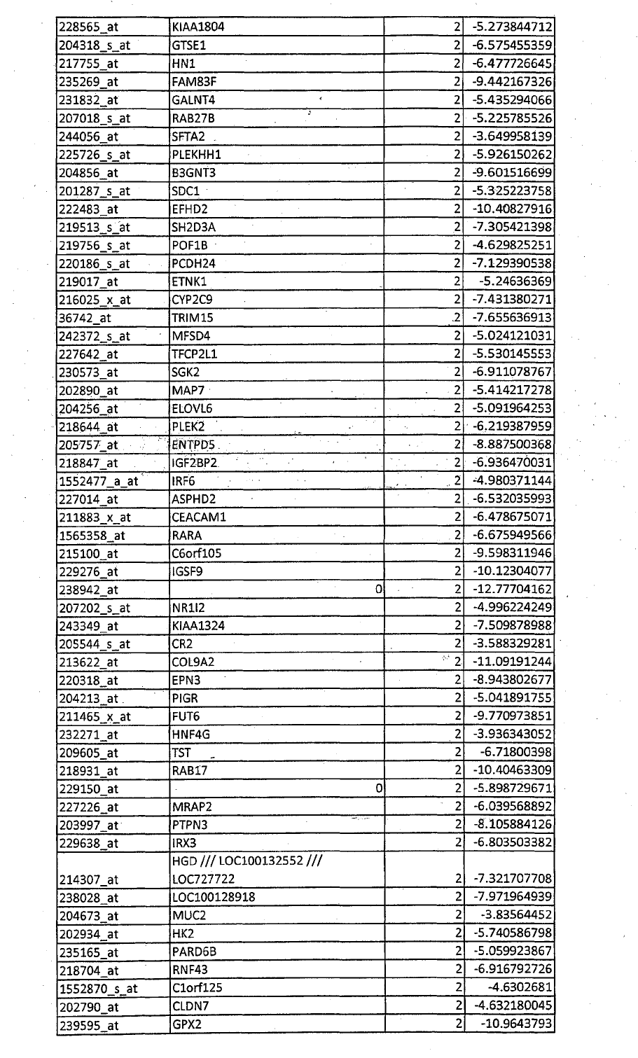

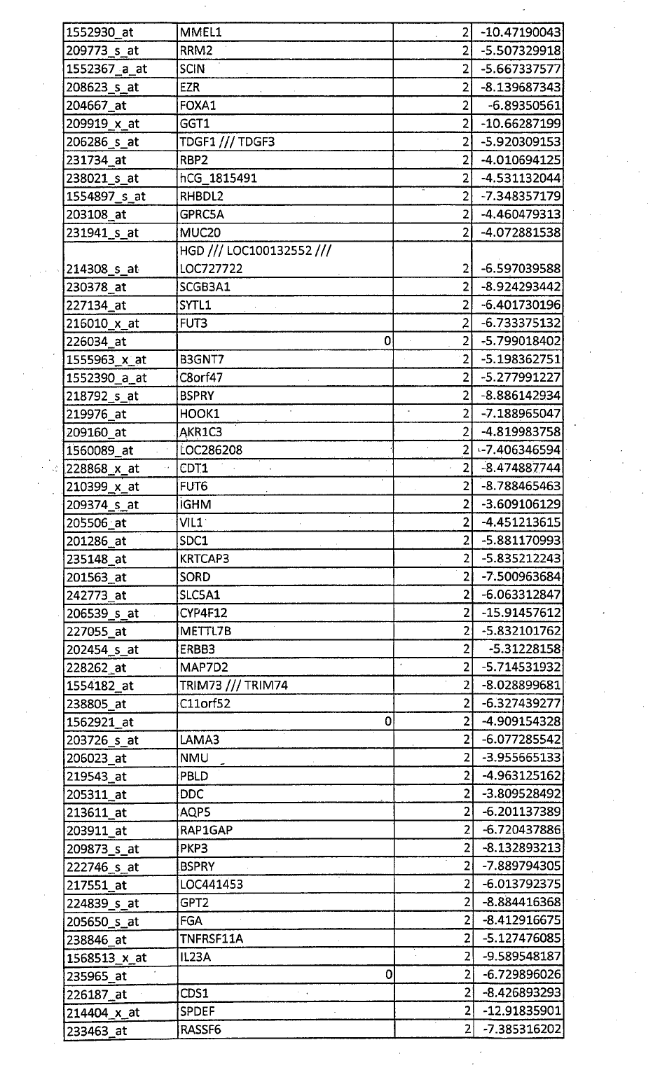

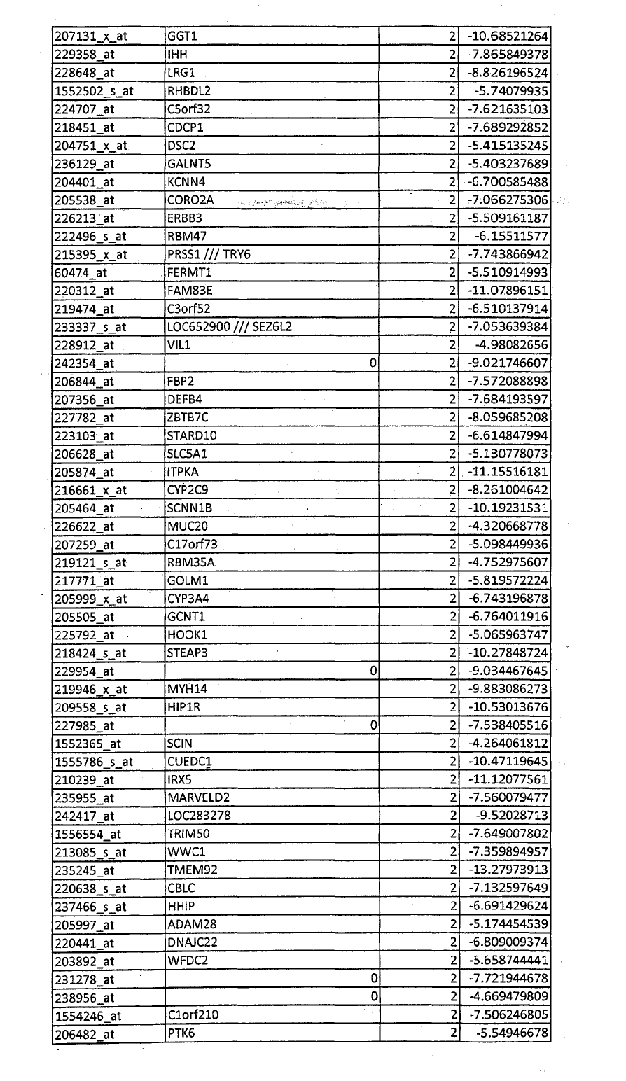

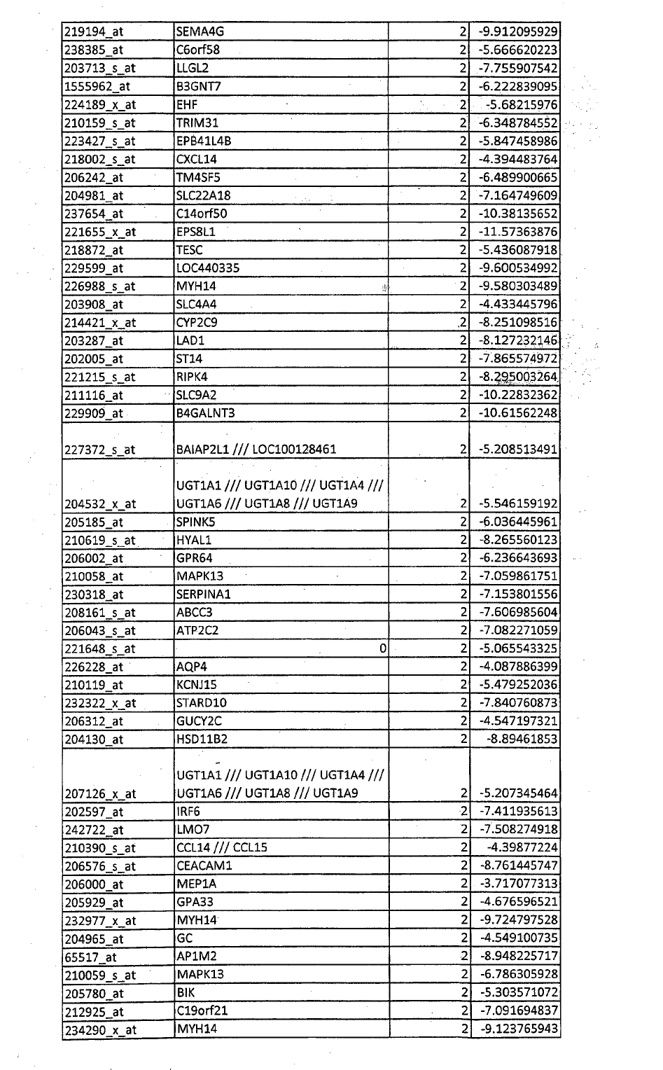

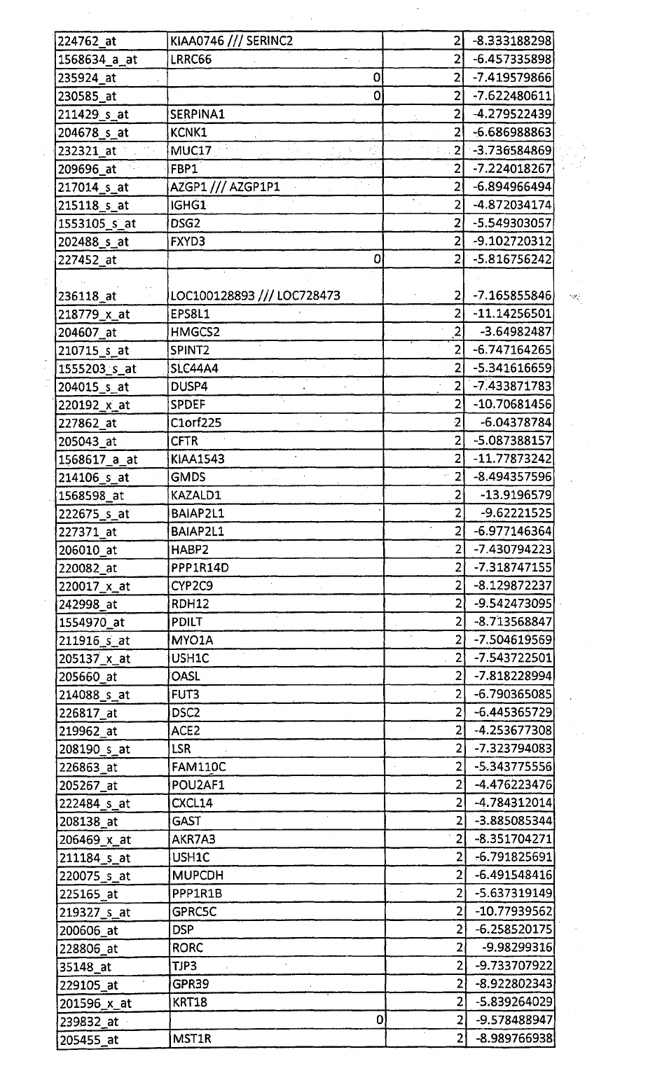

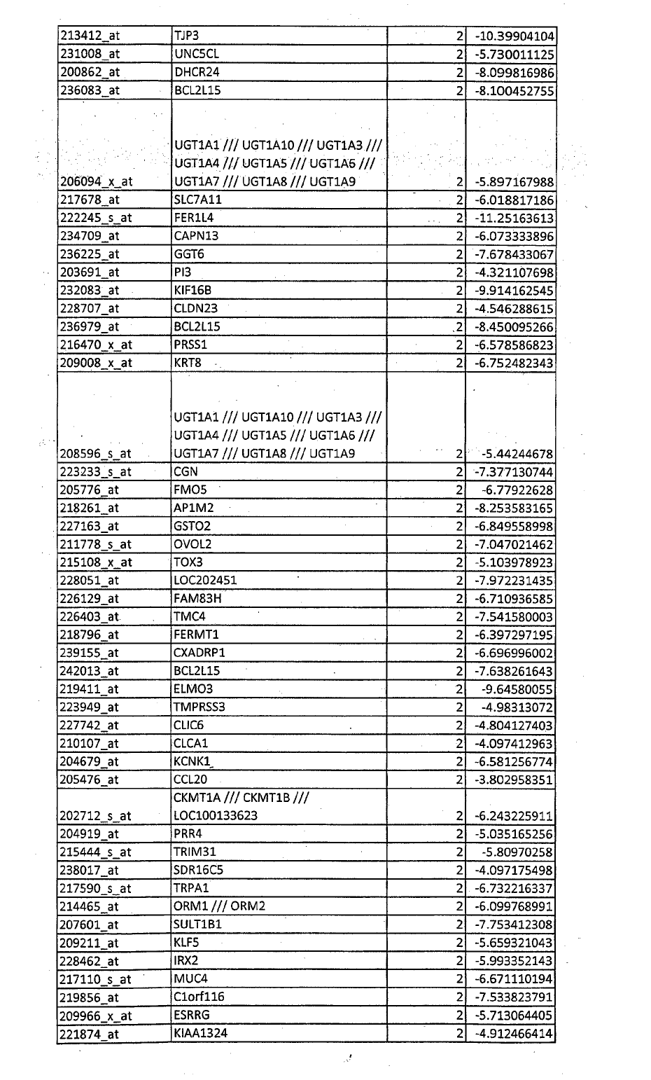

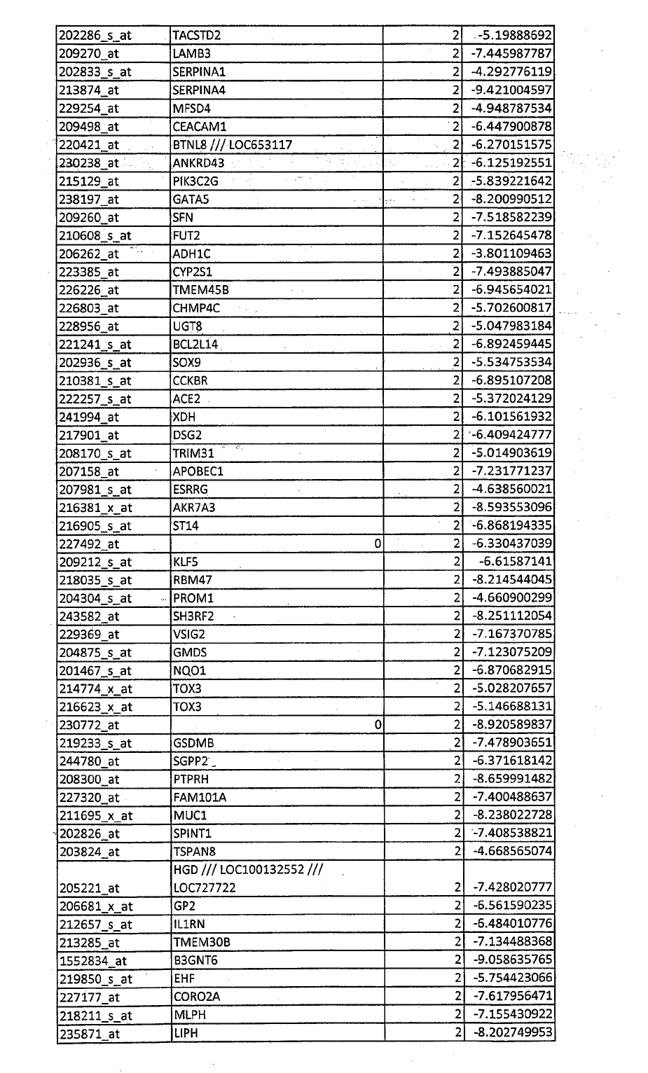

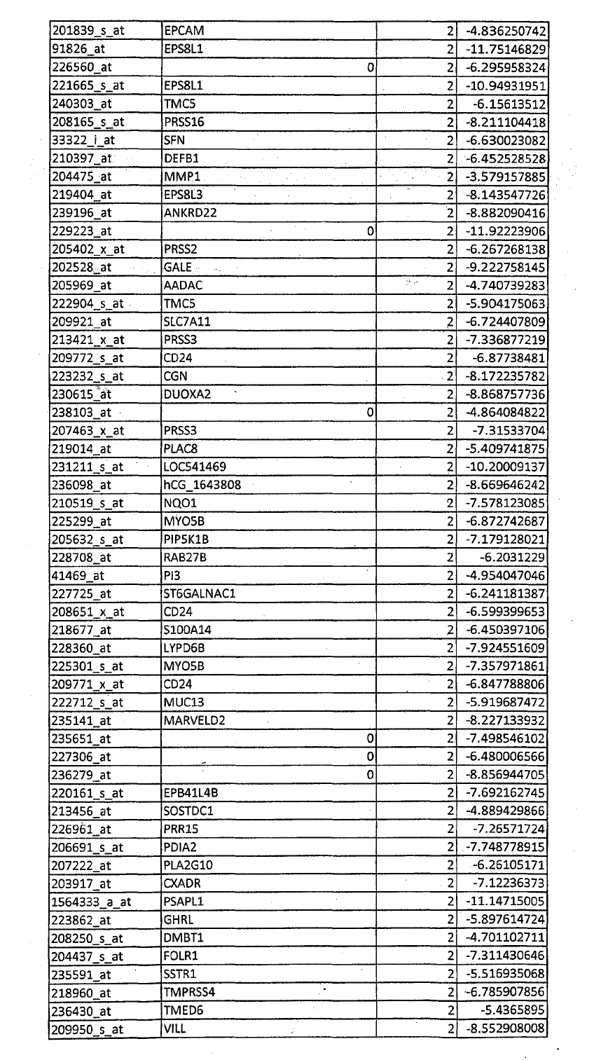

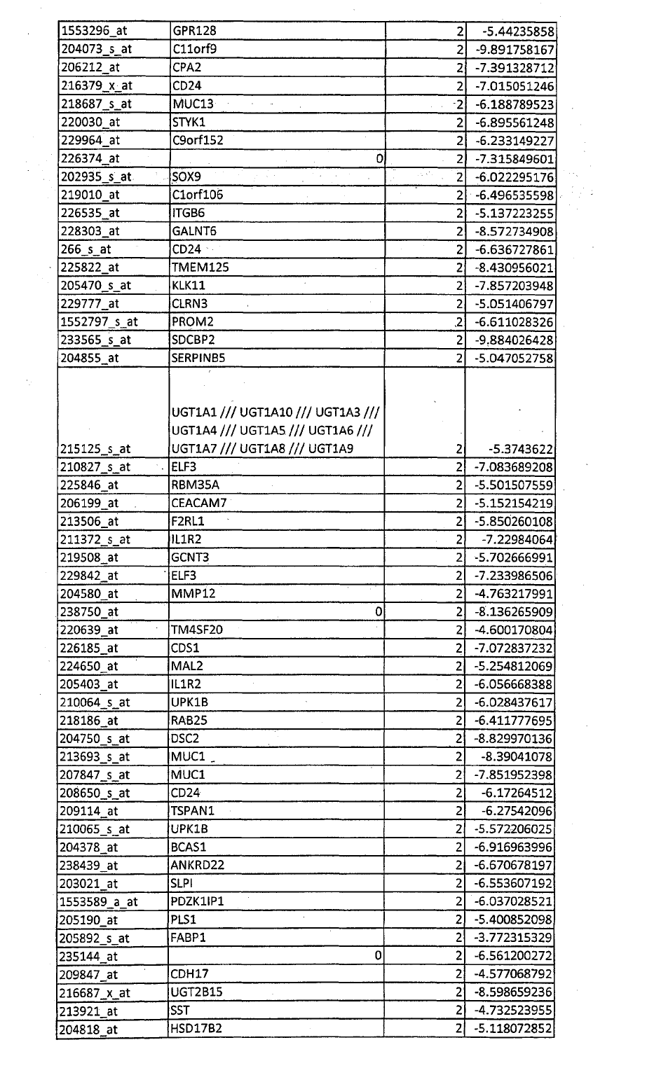

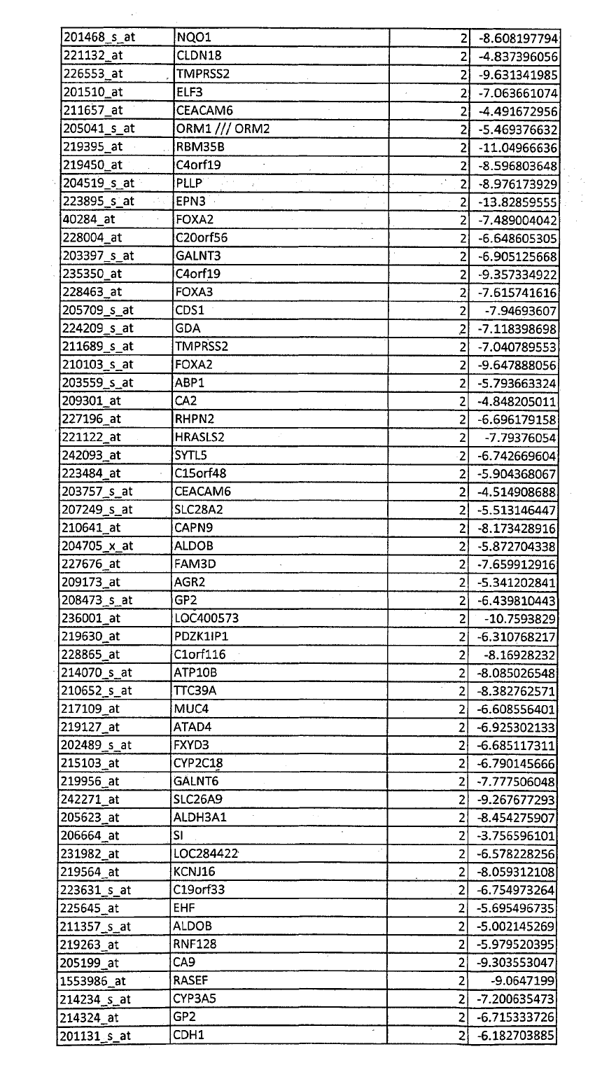

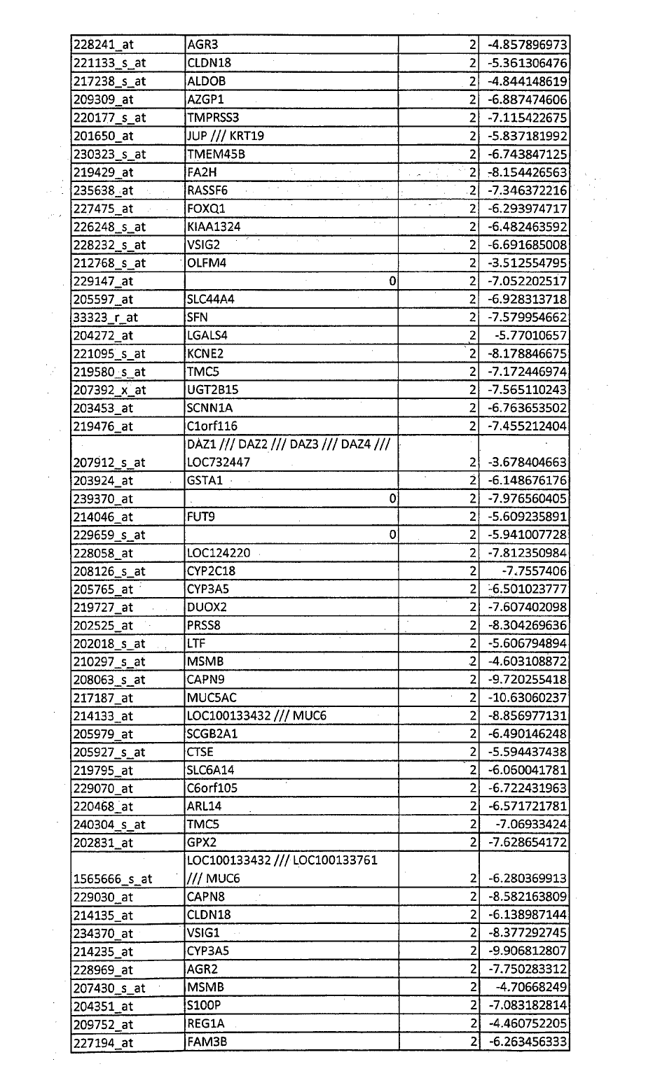

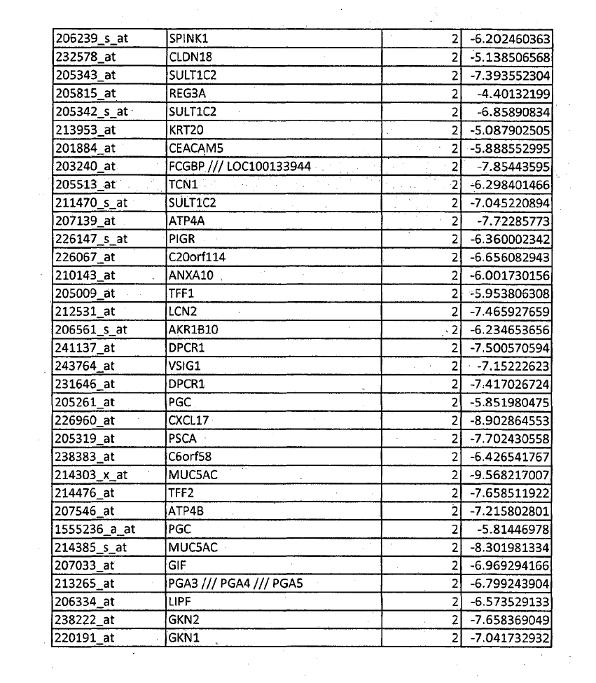

12. The predictor according to claim 8, wherein a second of the three predictors comprises a differentially expressed gene set comparing the differential expression between genes of the invasive subtype versus the metabolic subtype as shown in FIG. 19.

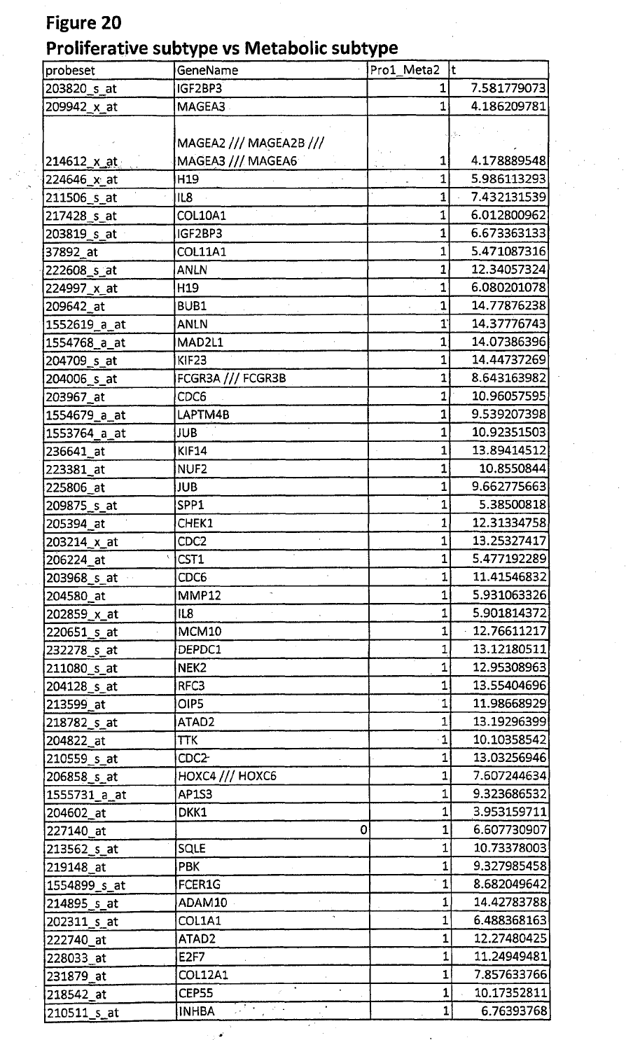

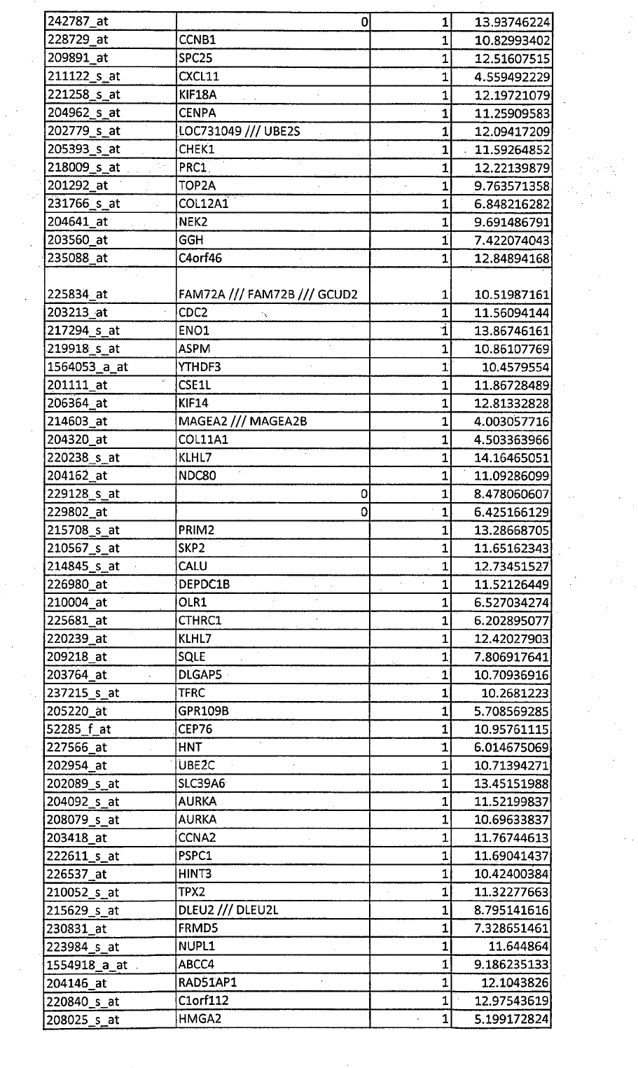

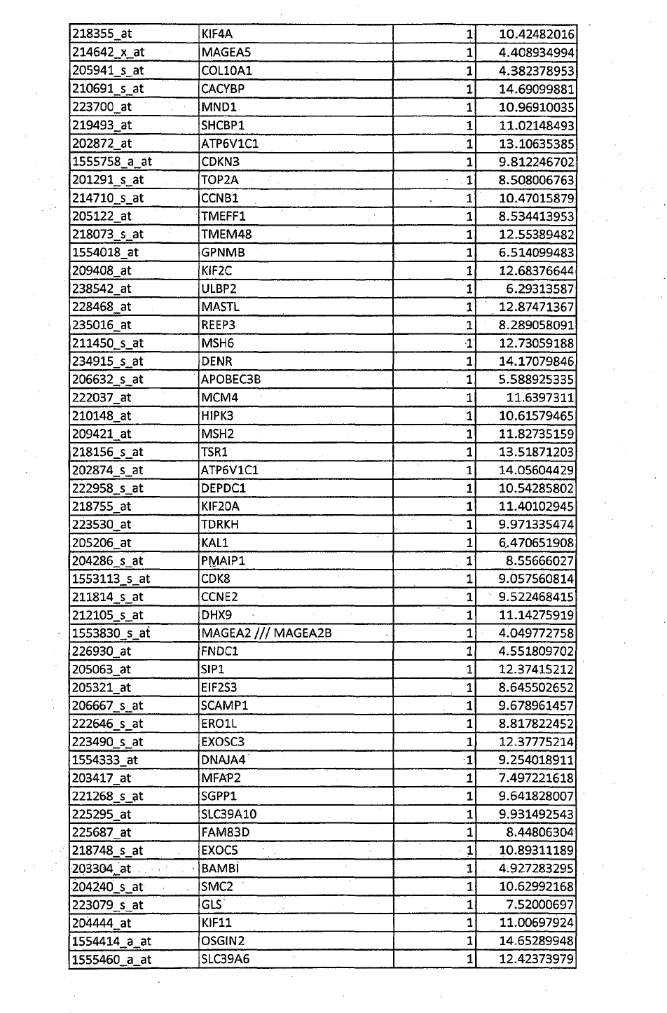

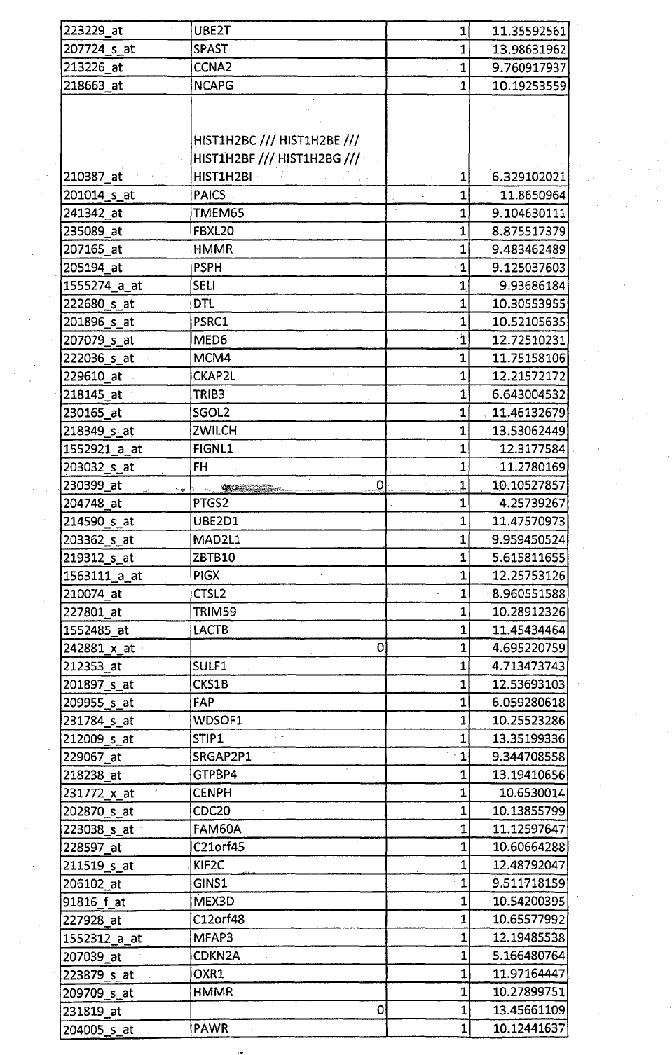

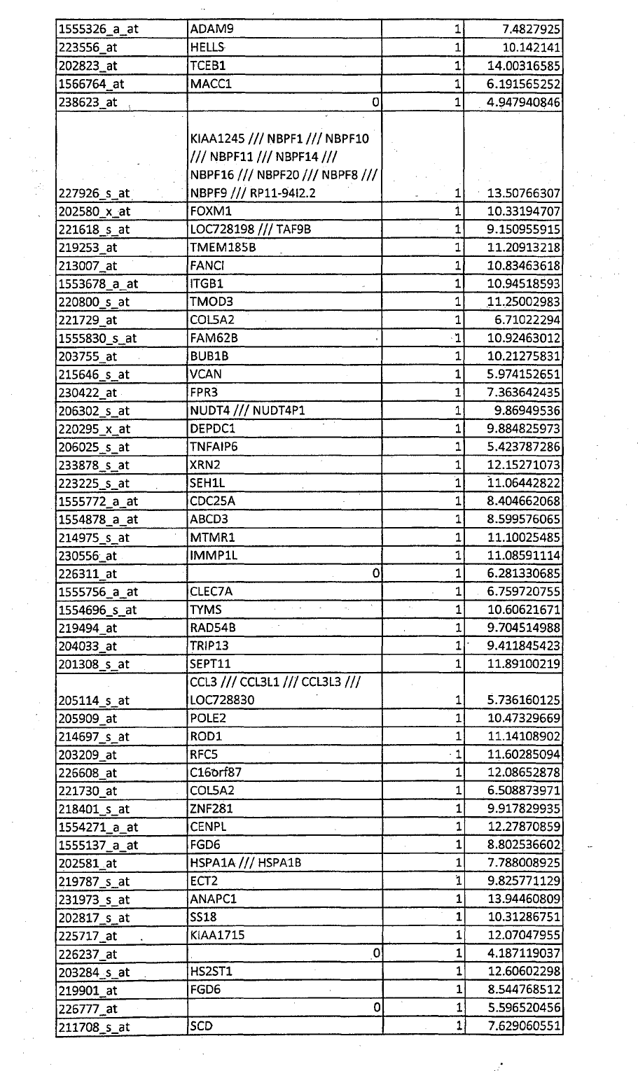

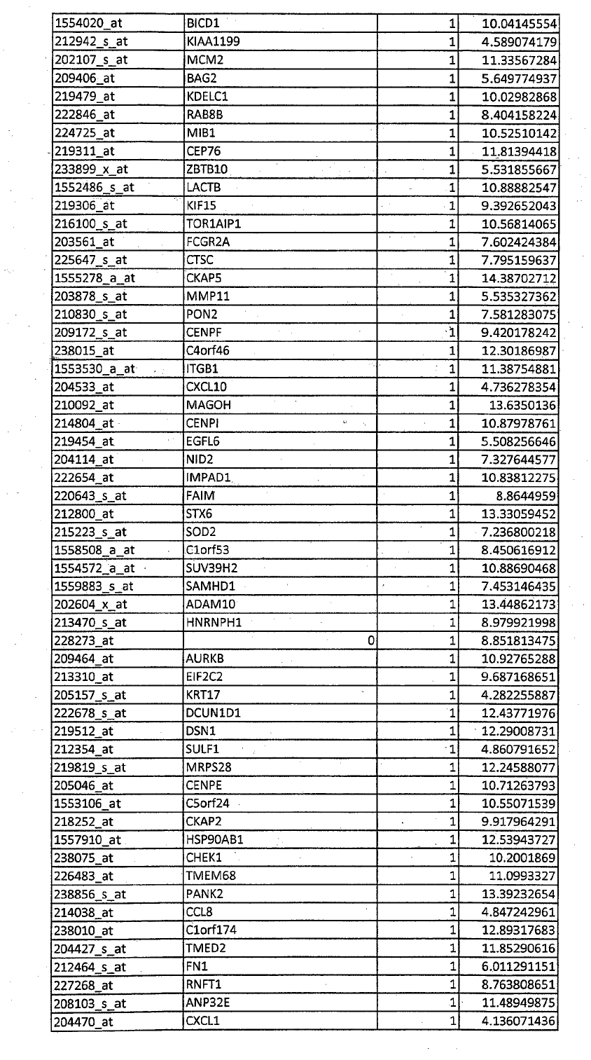

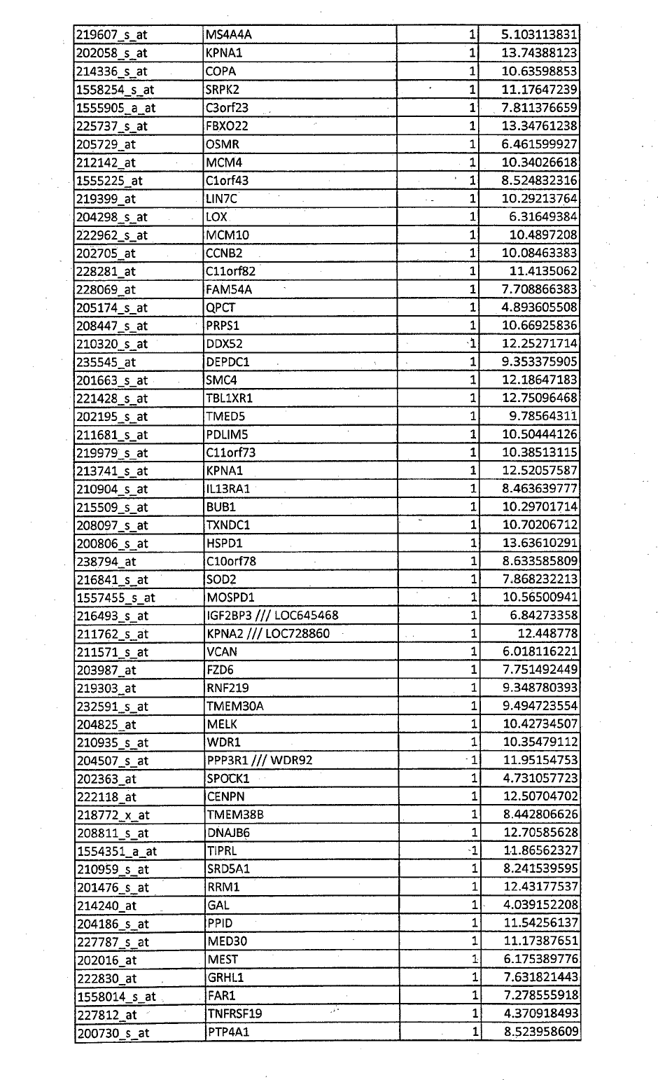

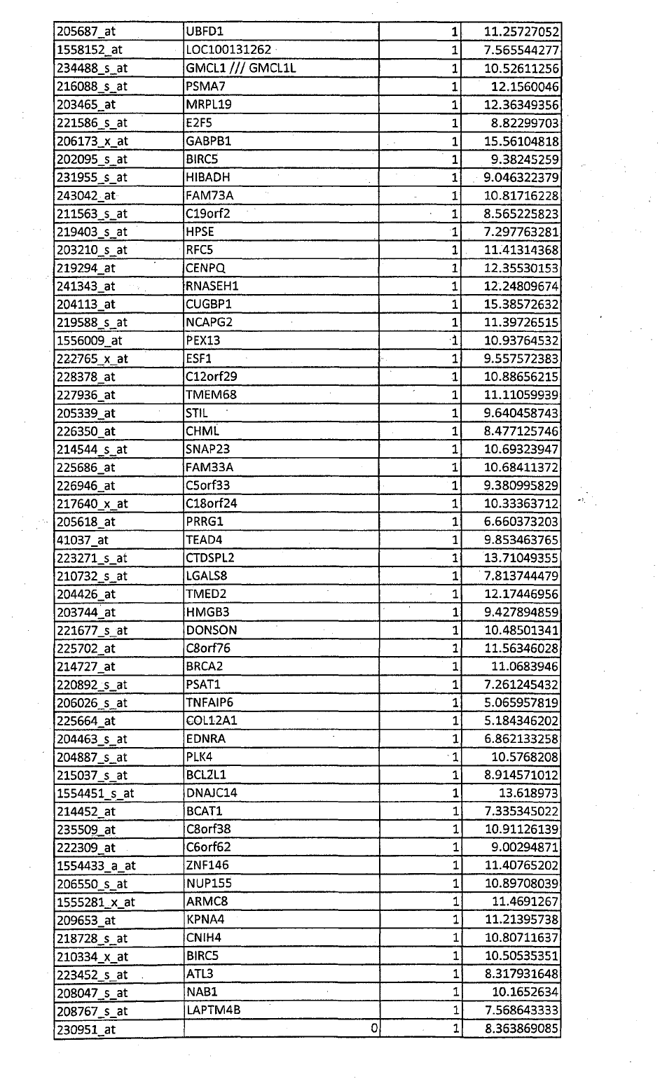

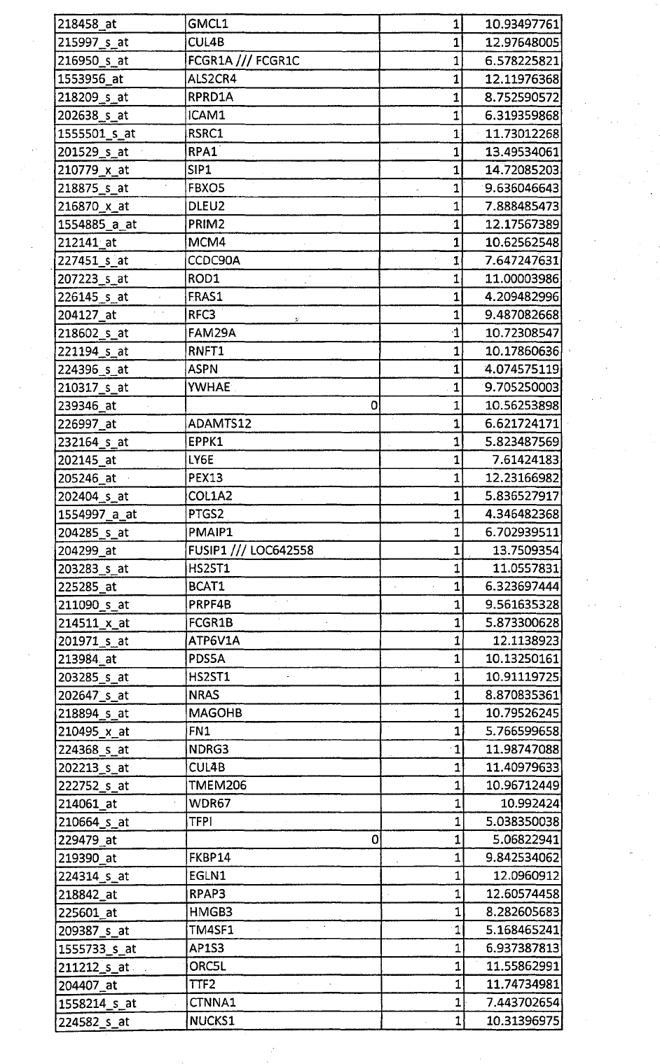

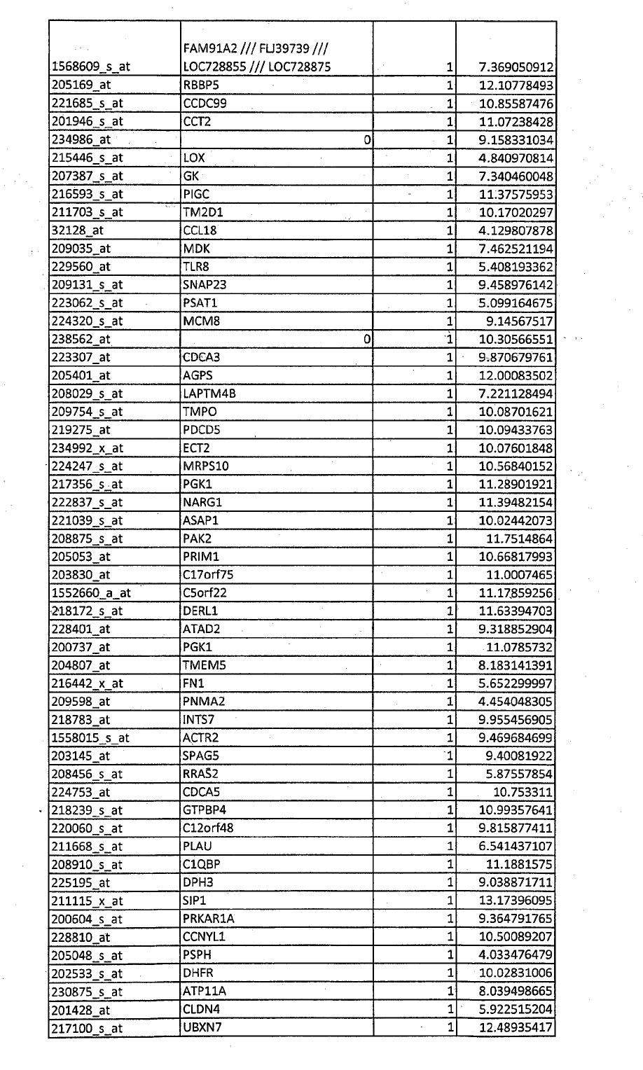

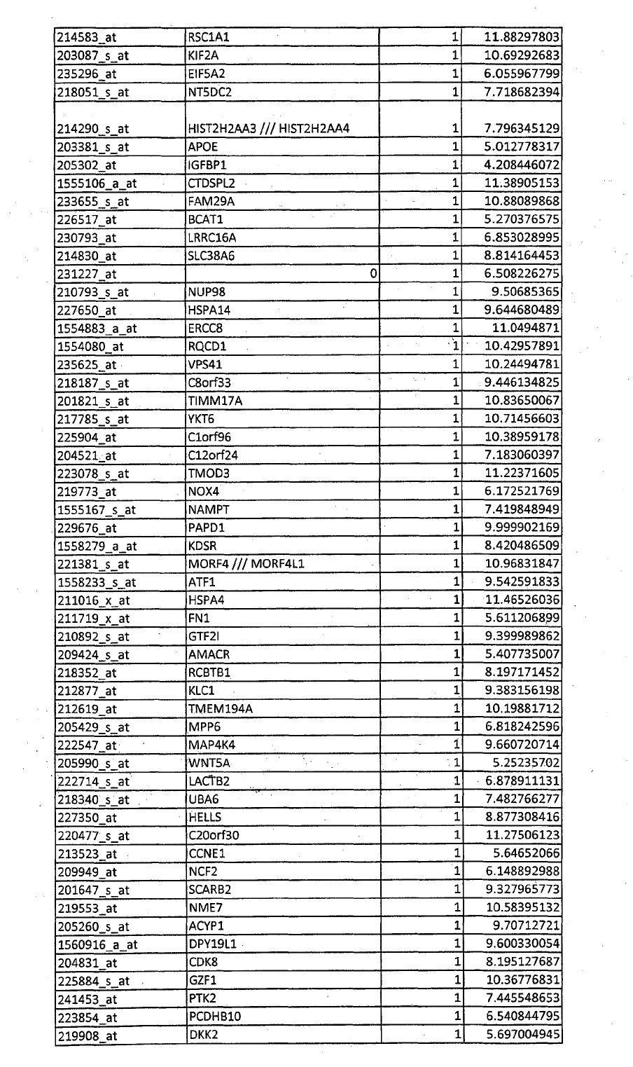

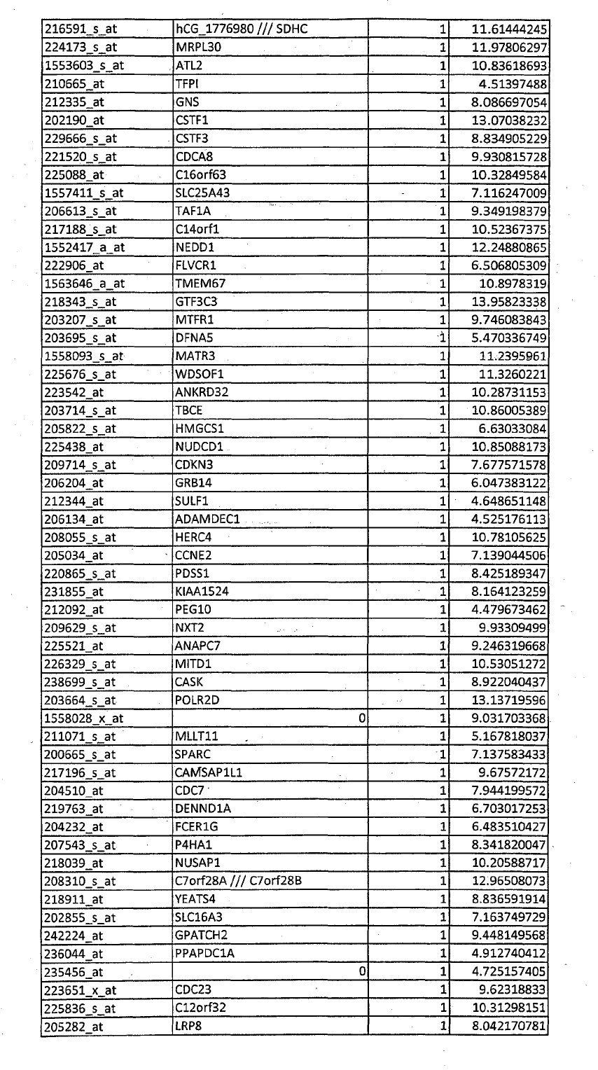

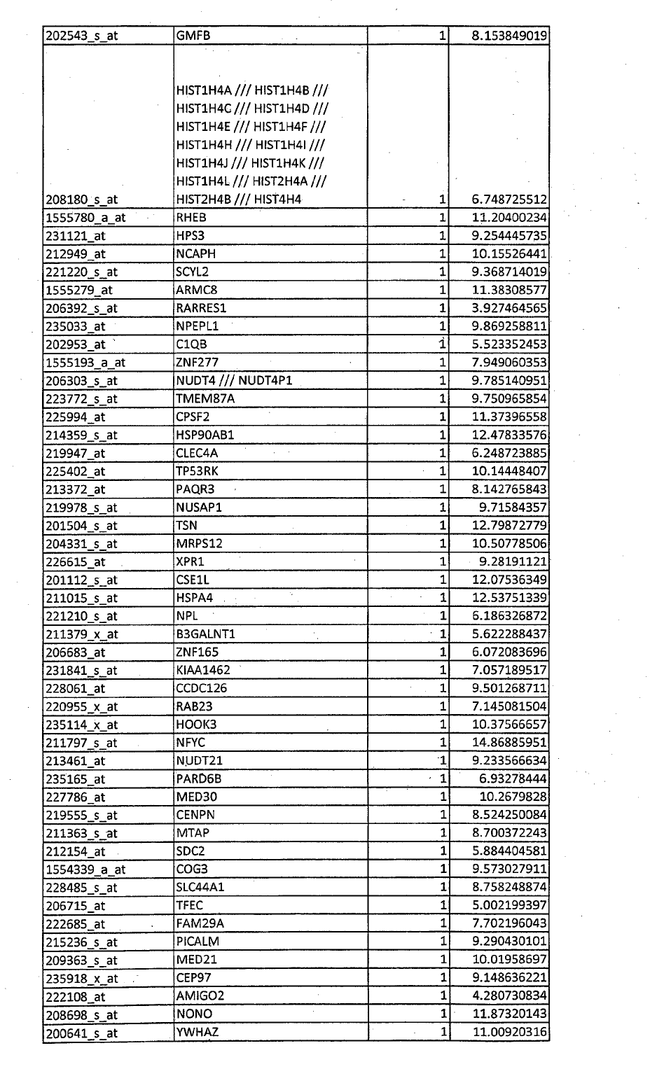

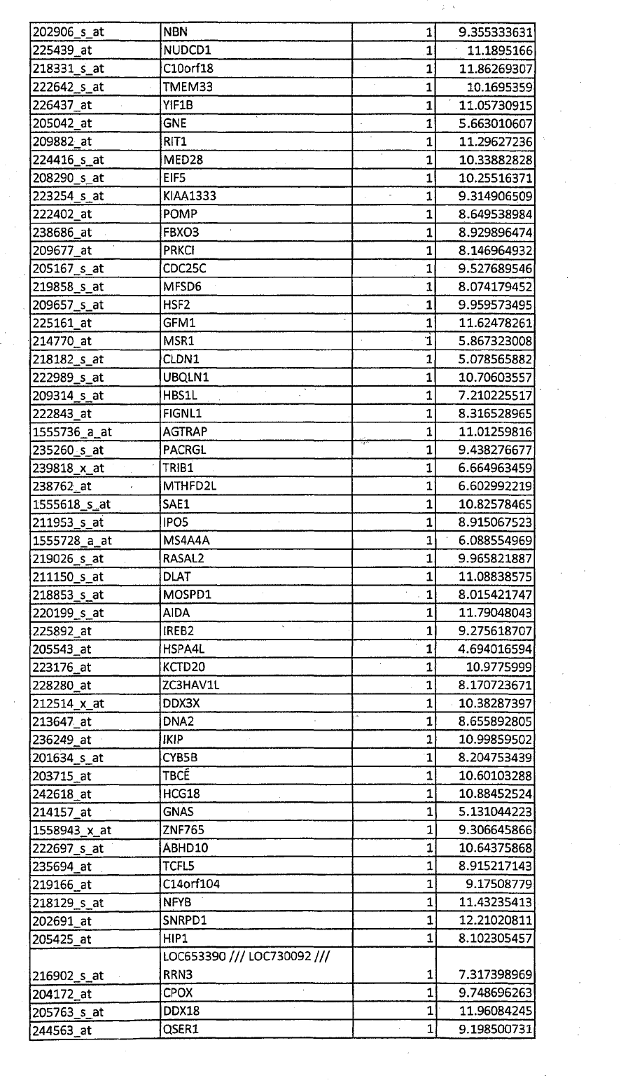

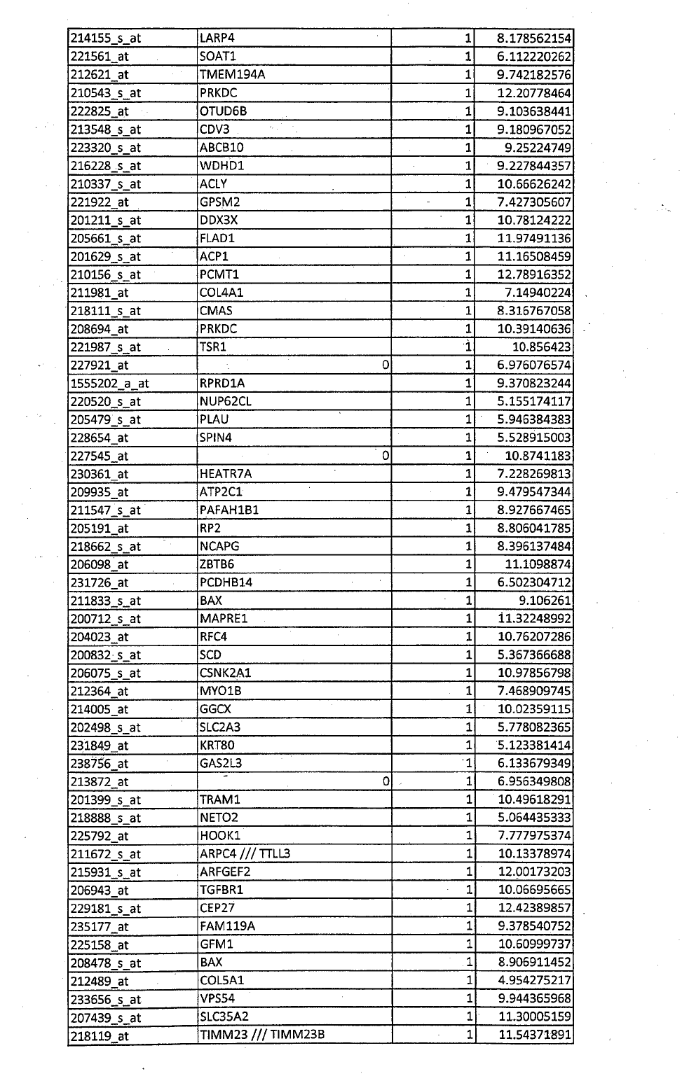

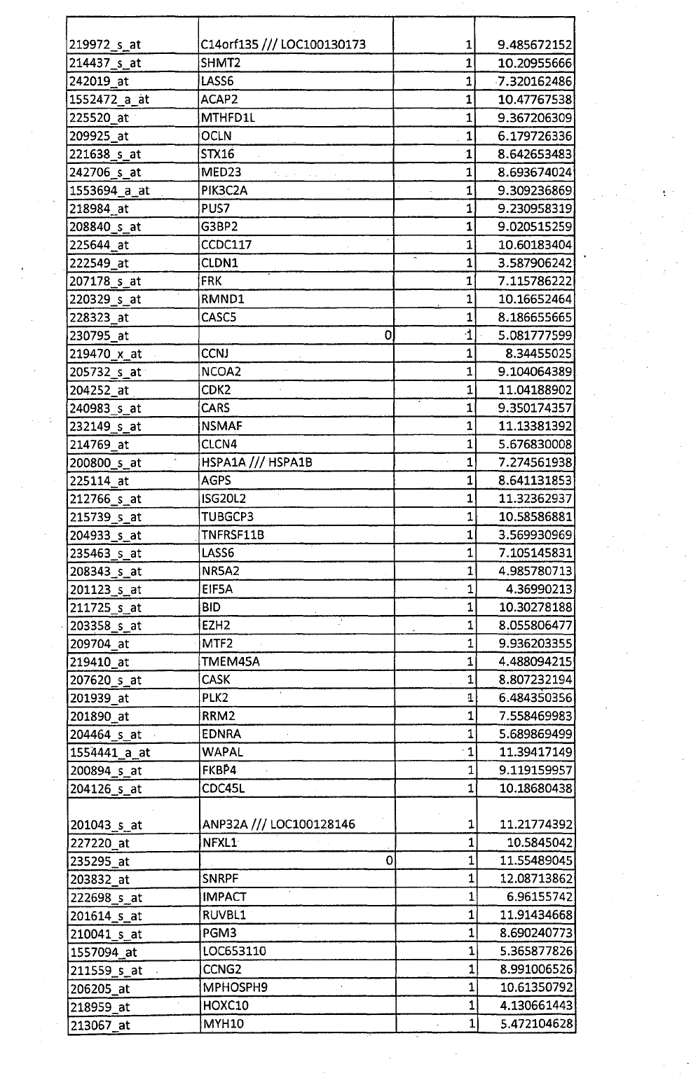

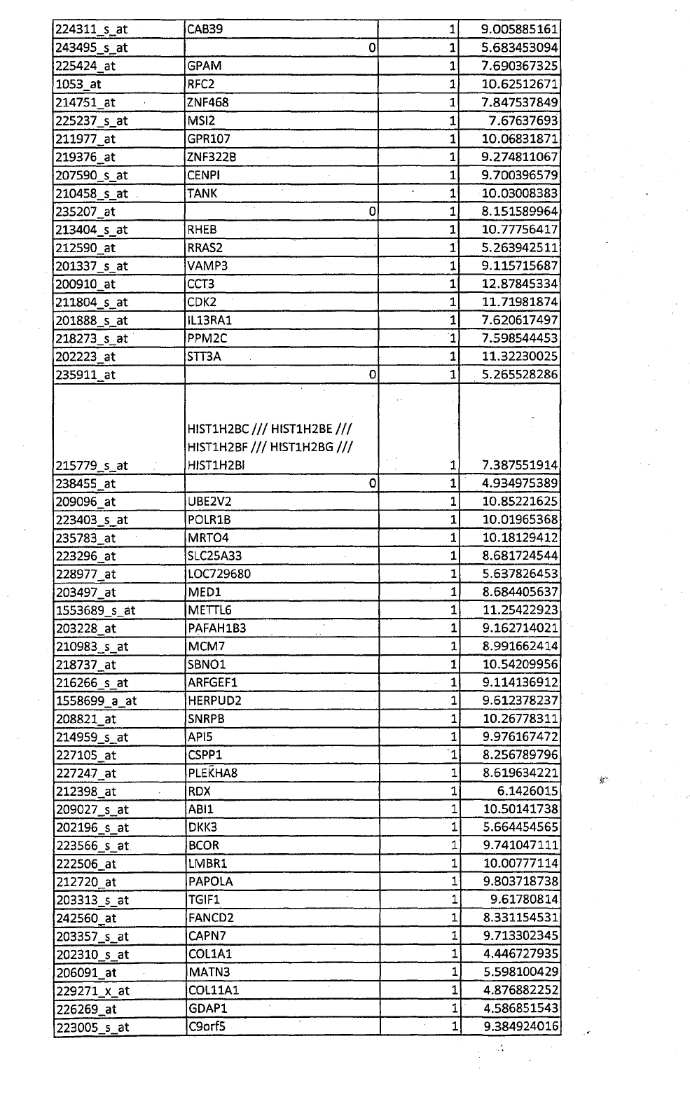

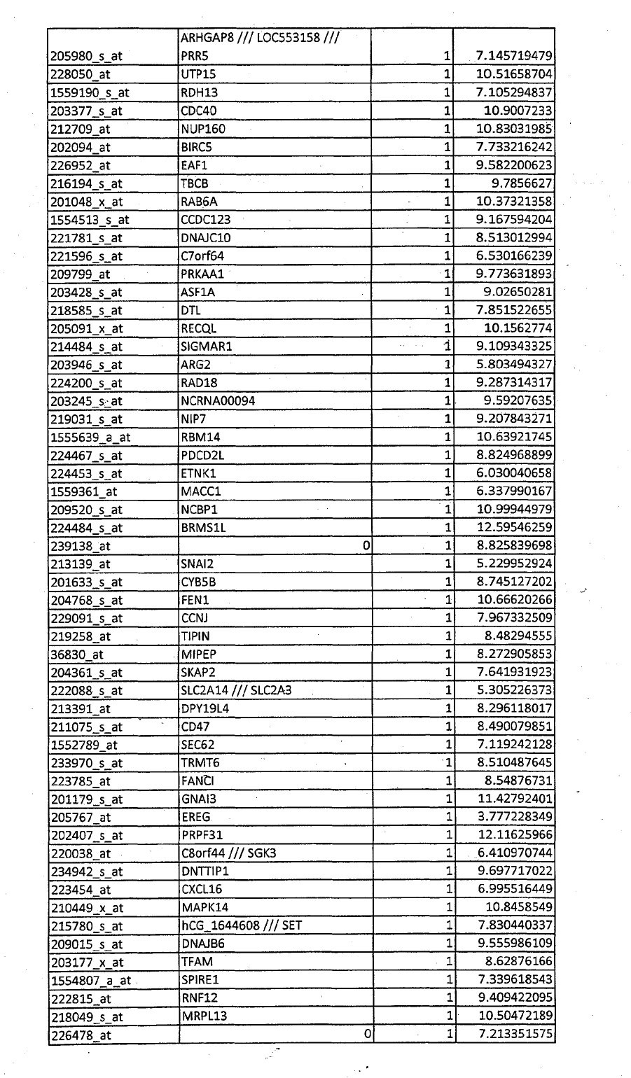

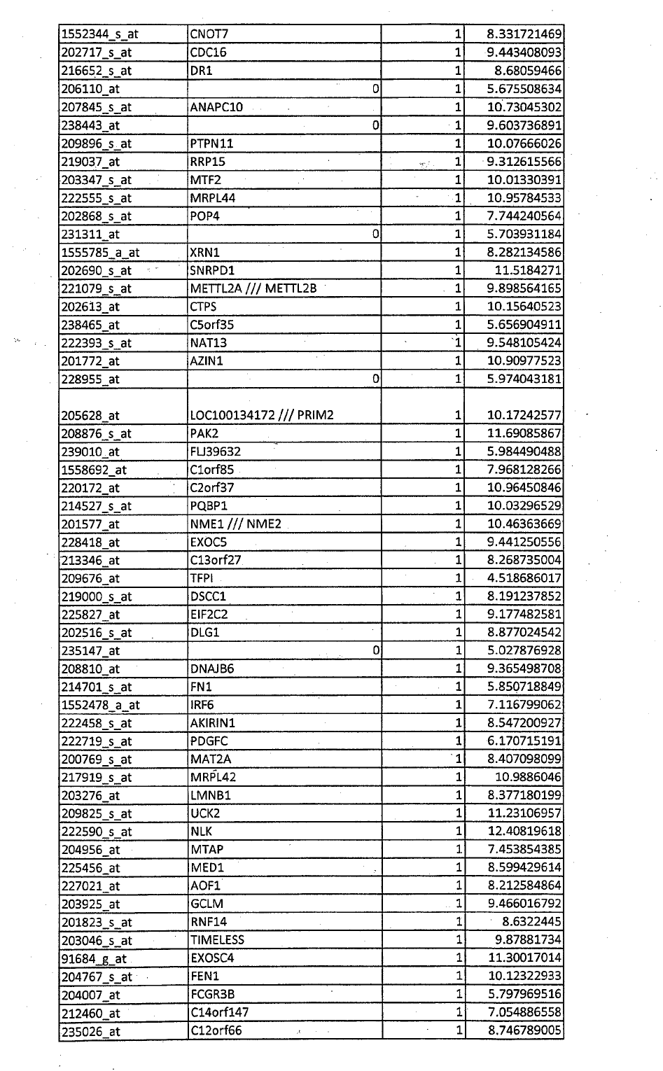

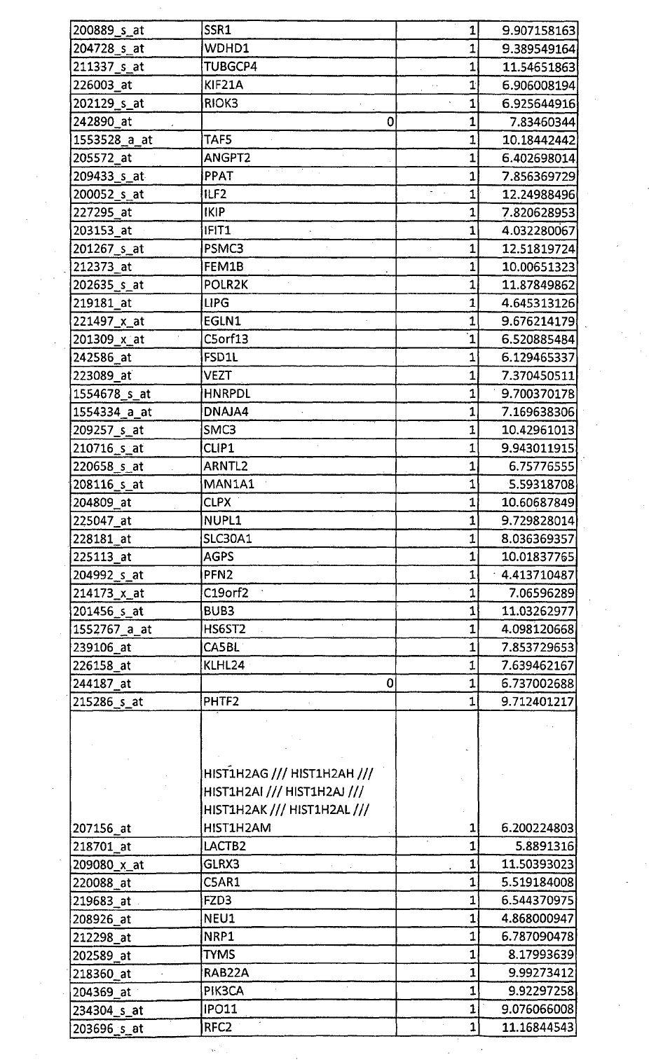

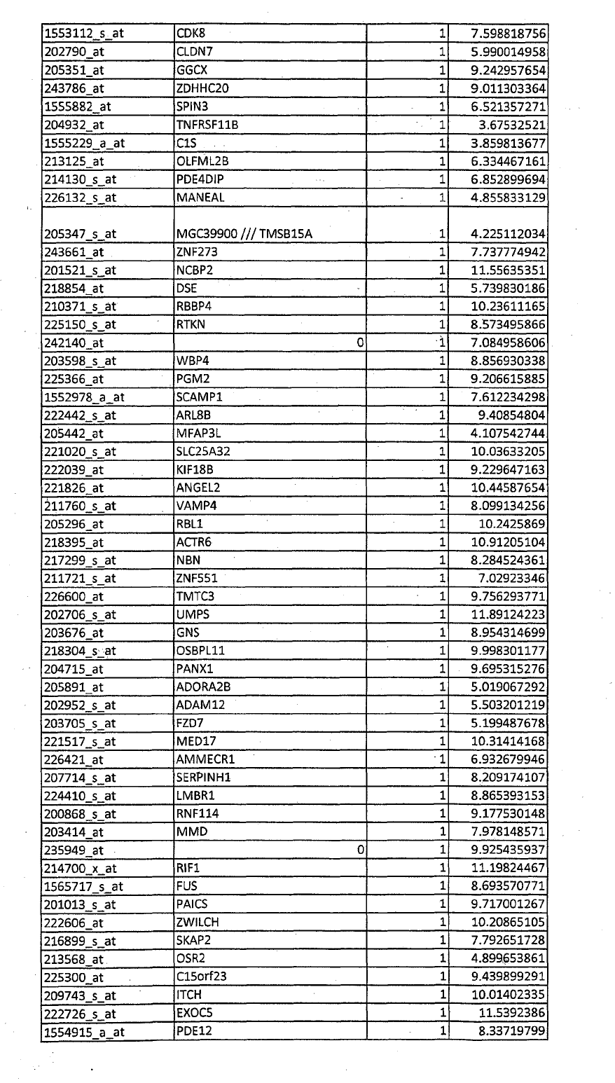

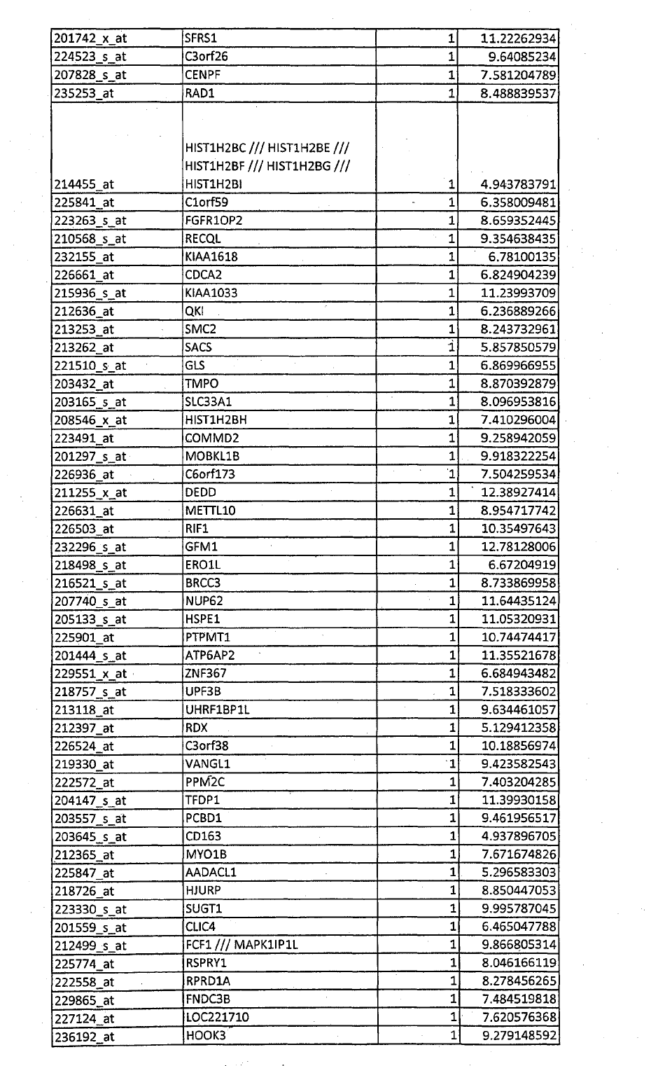

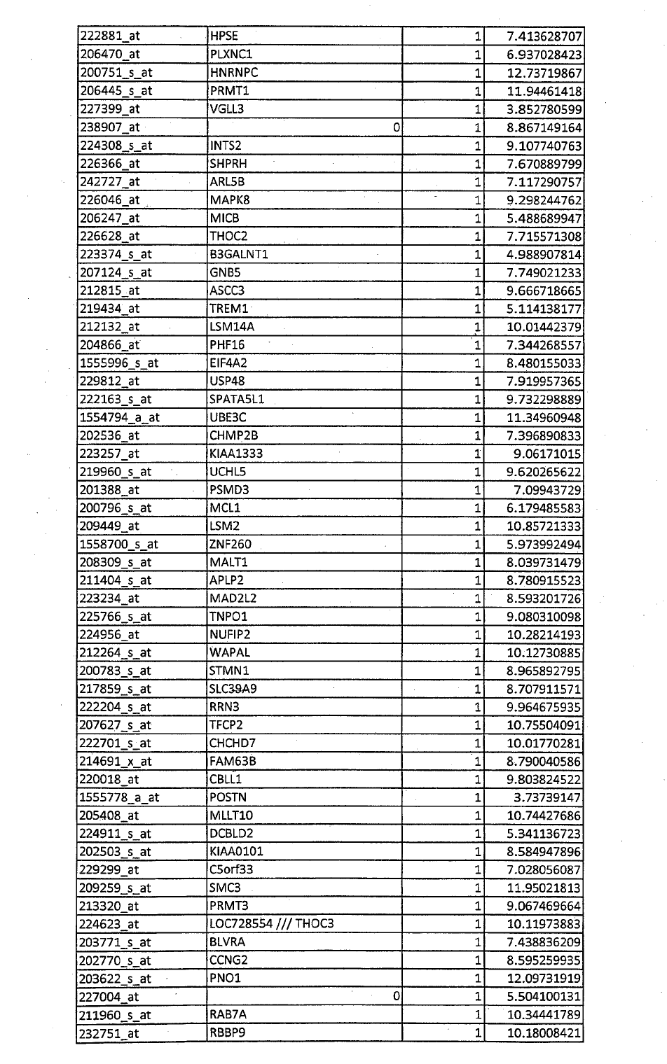

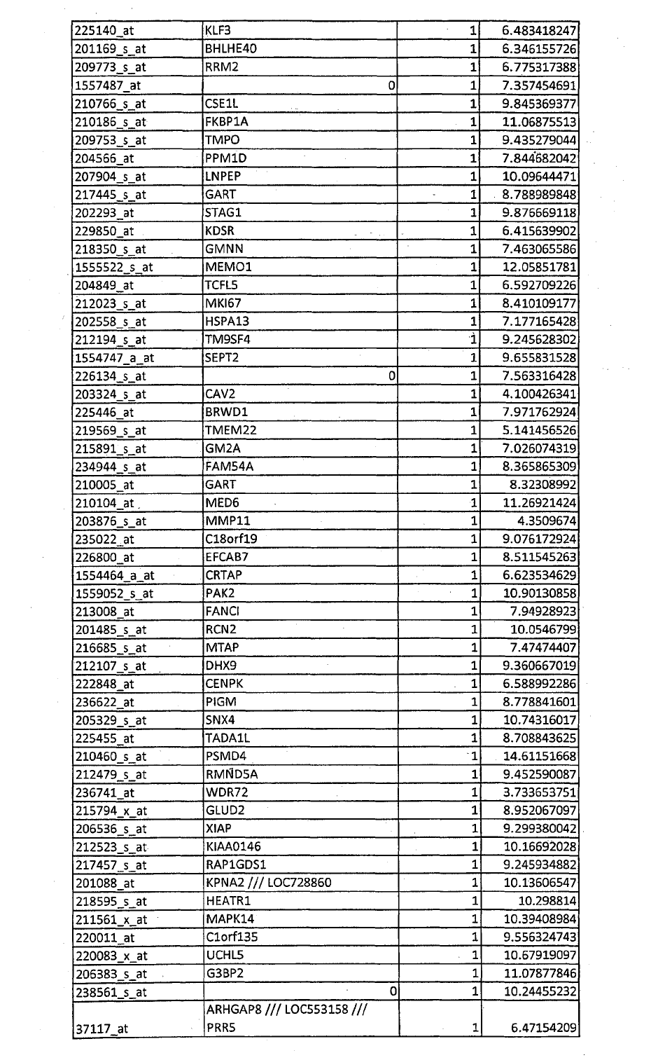

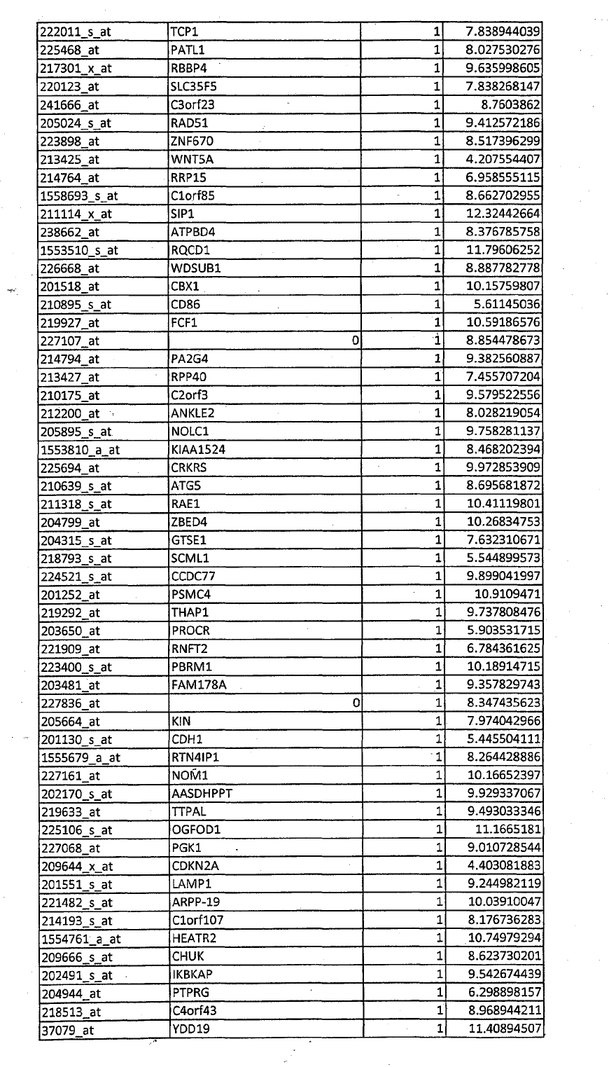

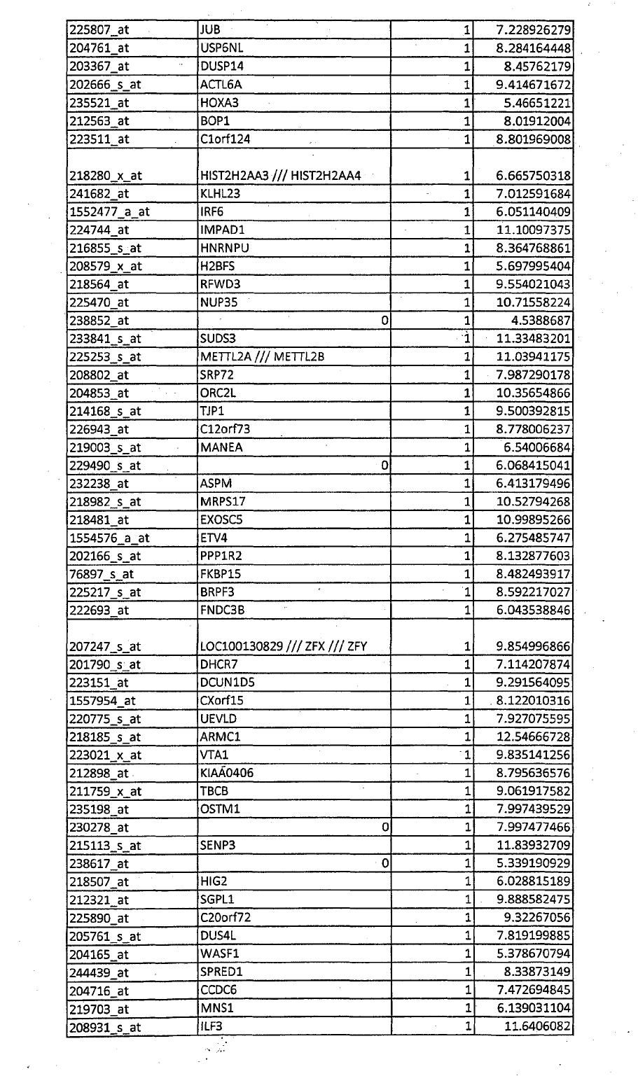

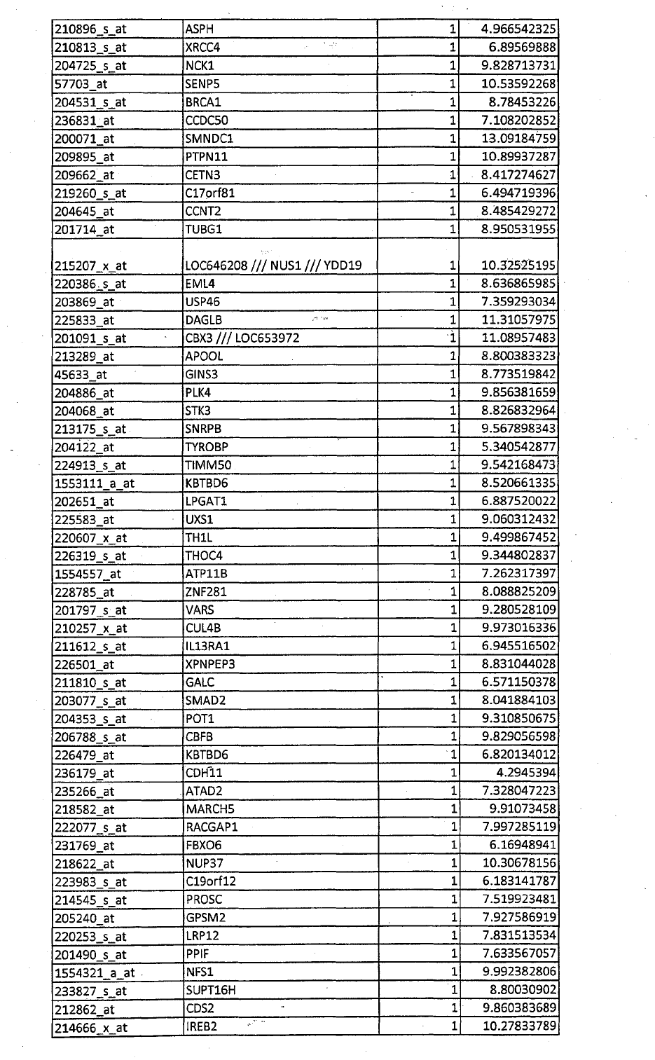

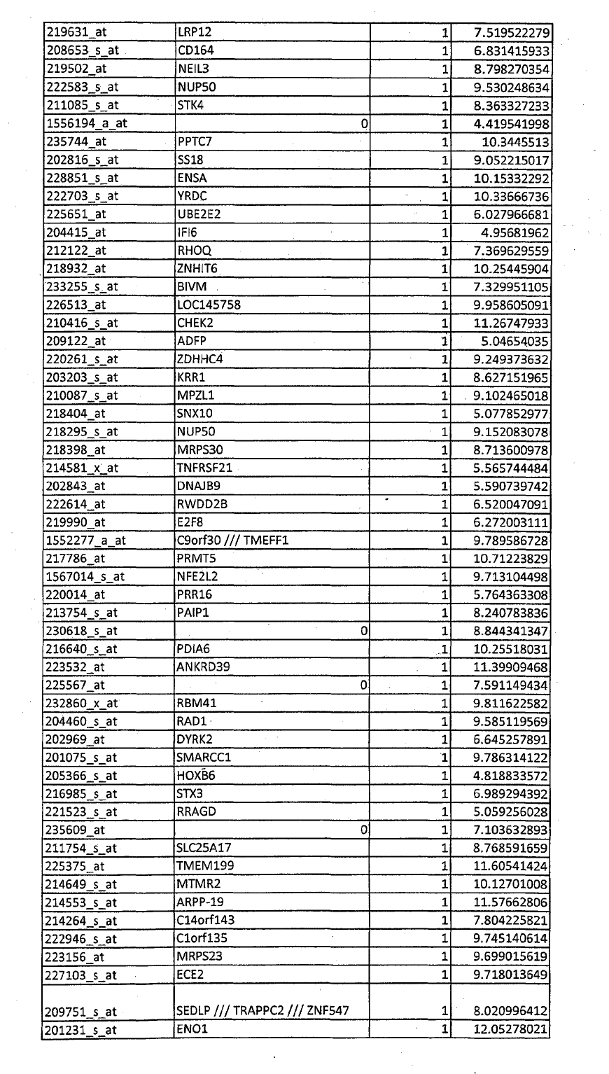

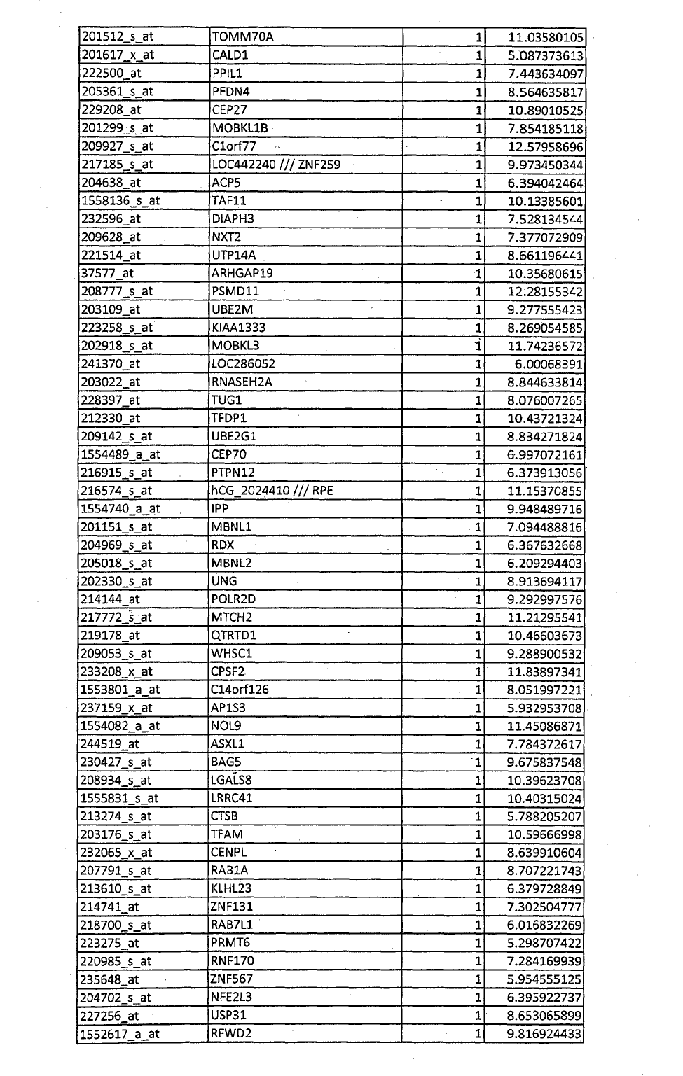

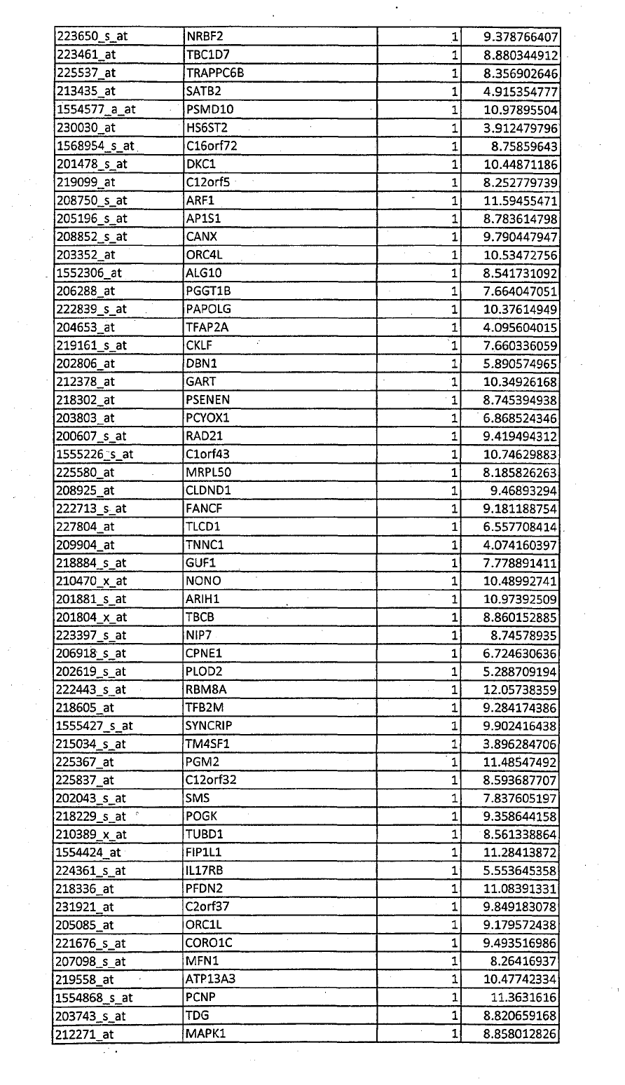

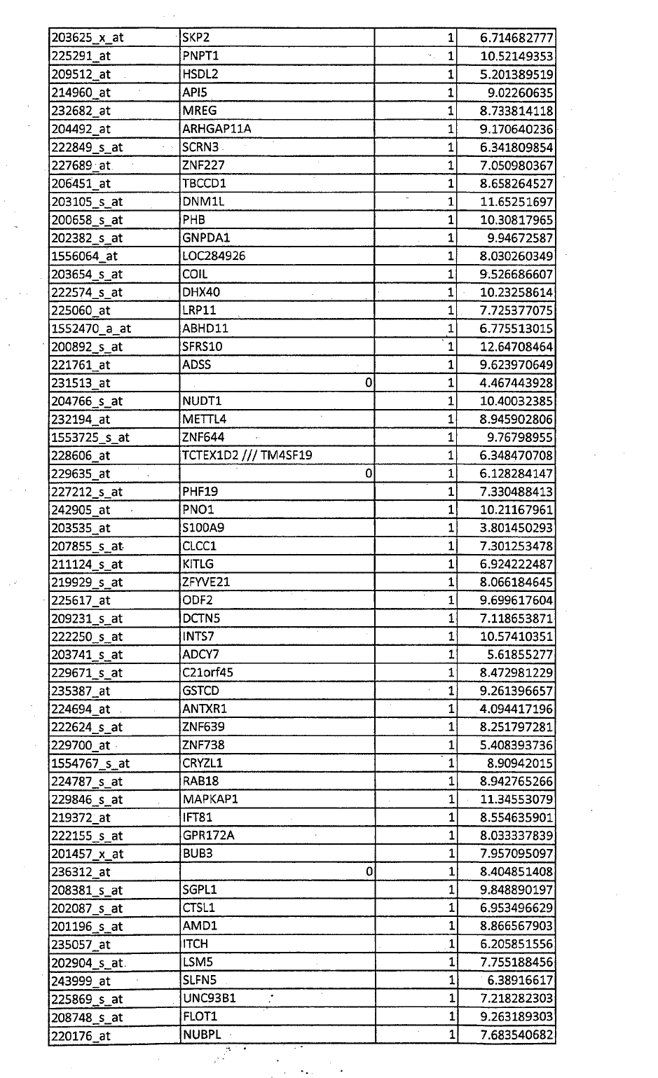

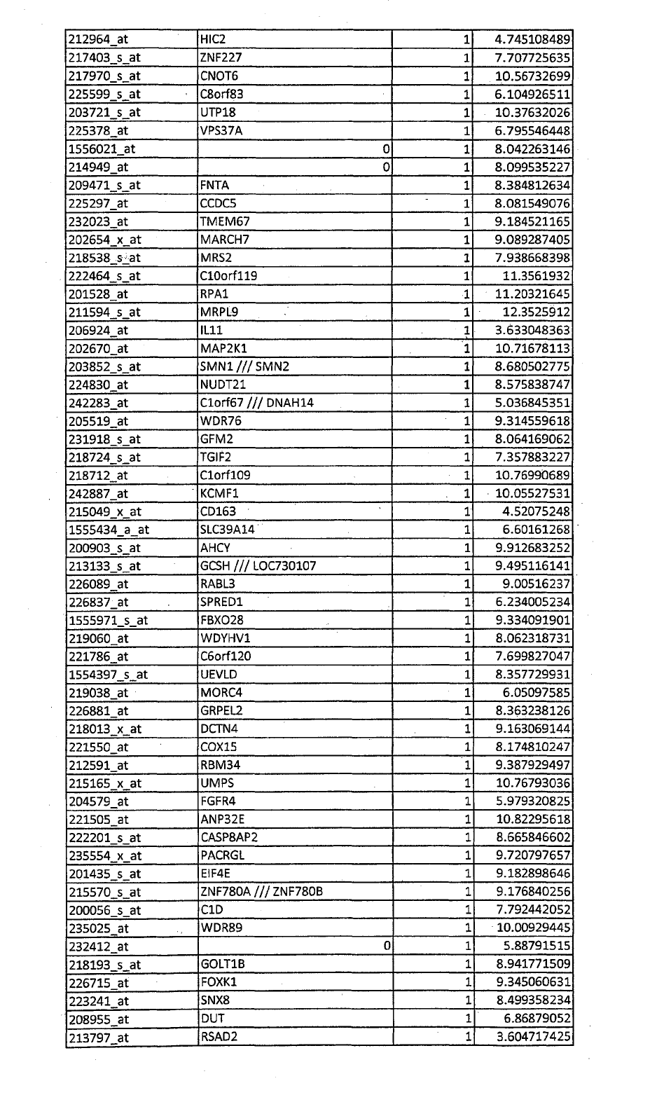

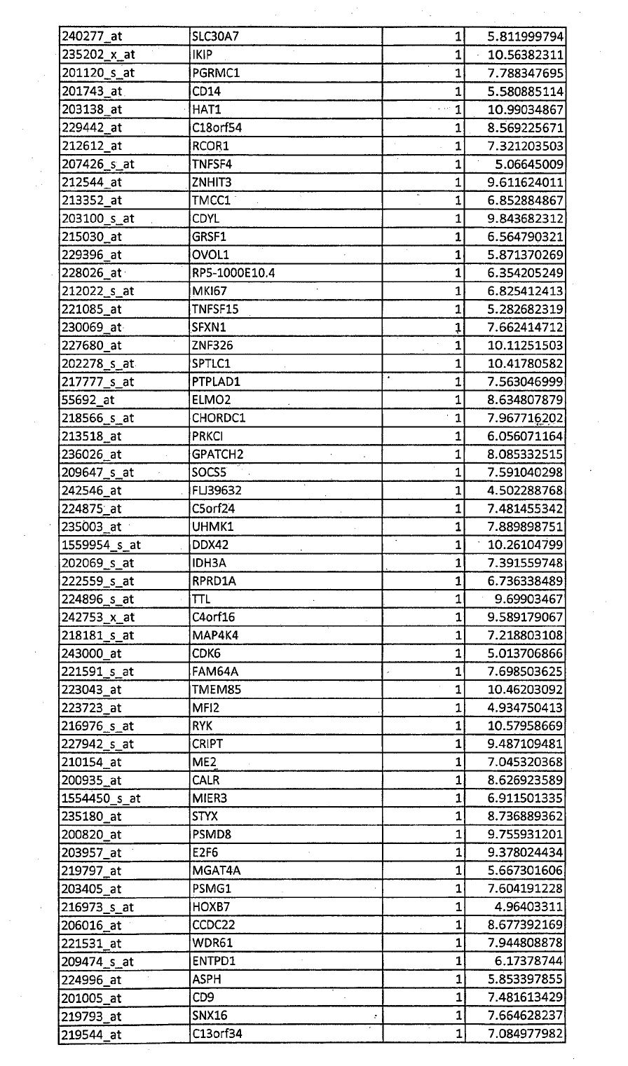

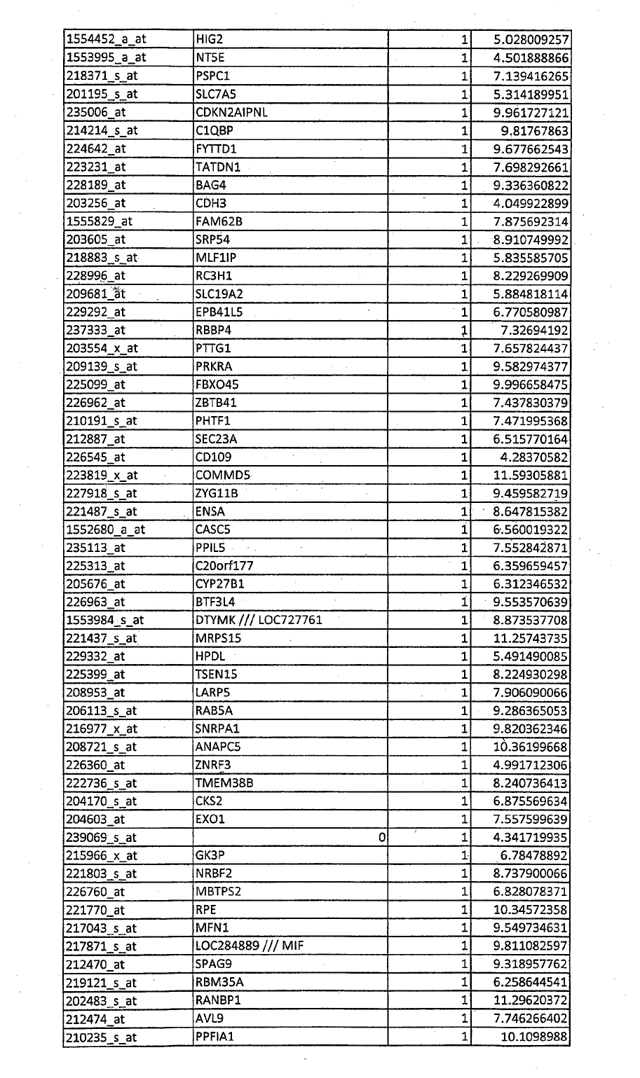

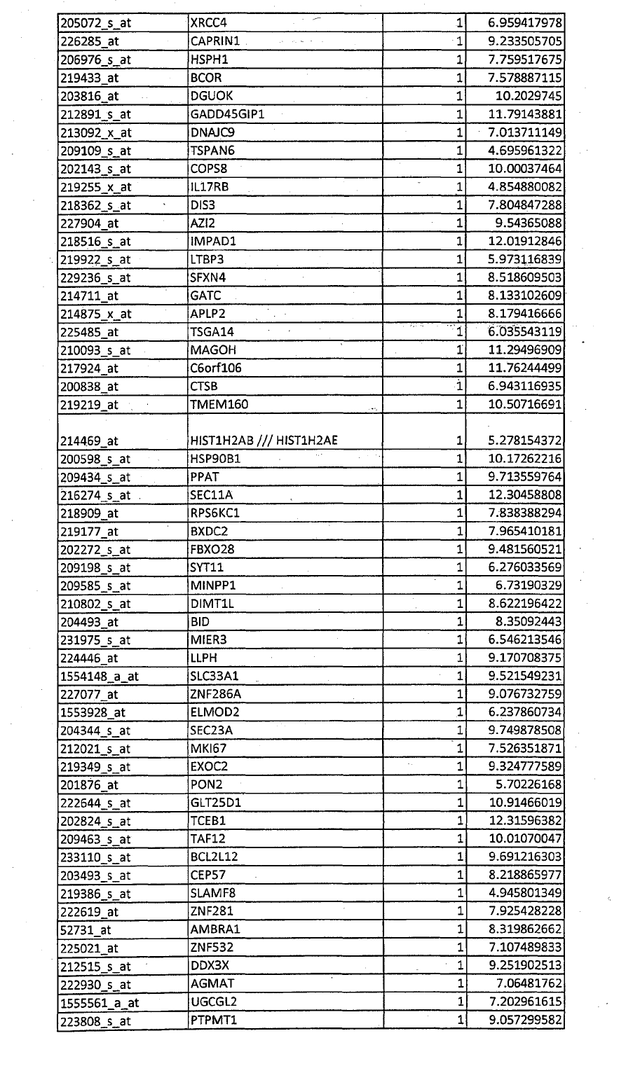

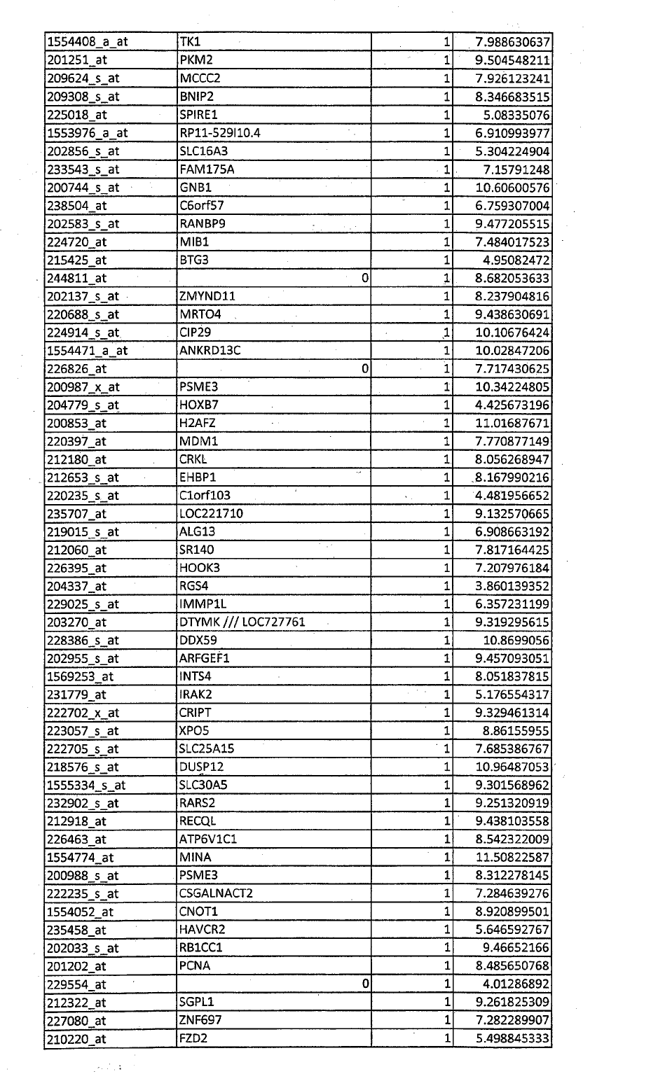

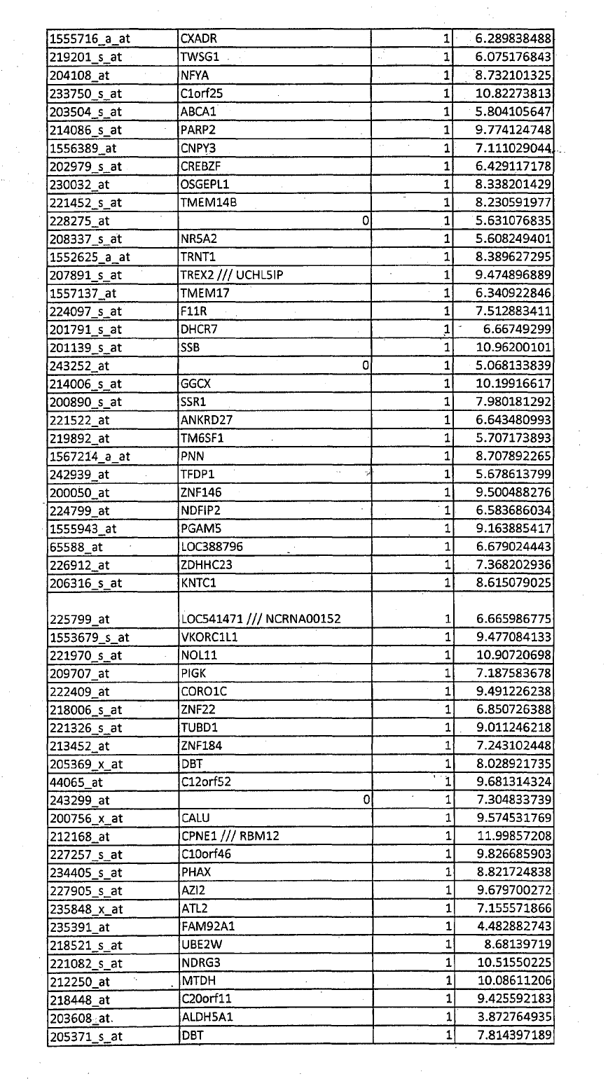

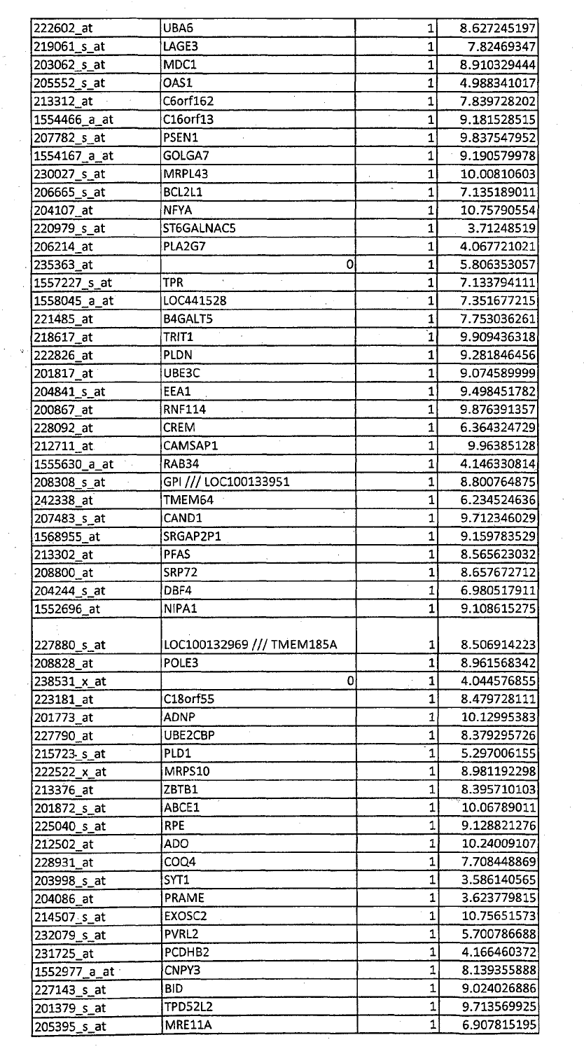

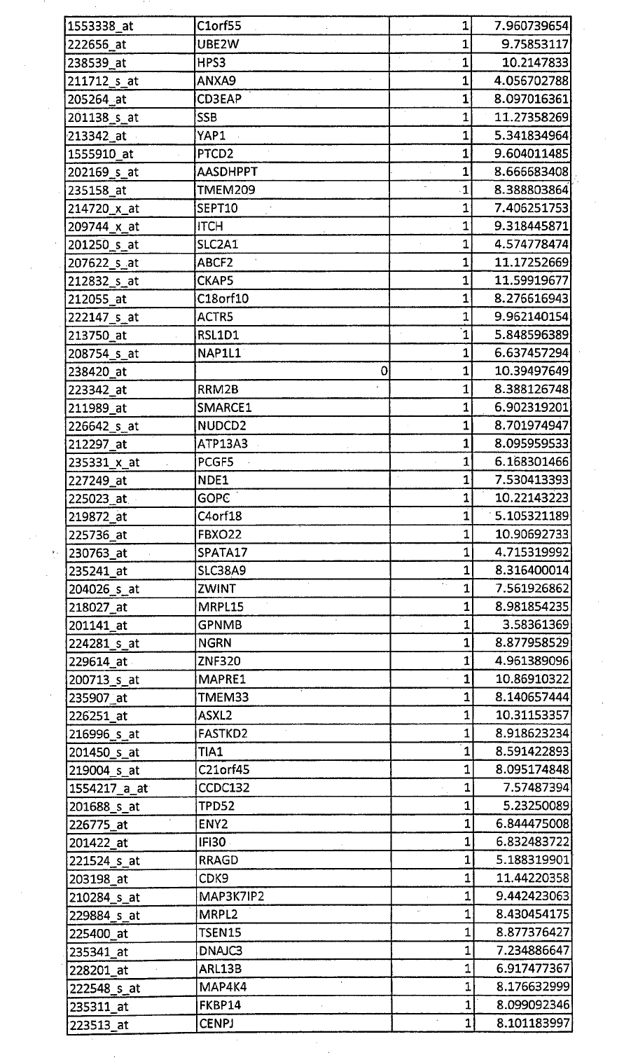

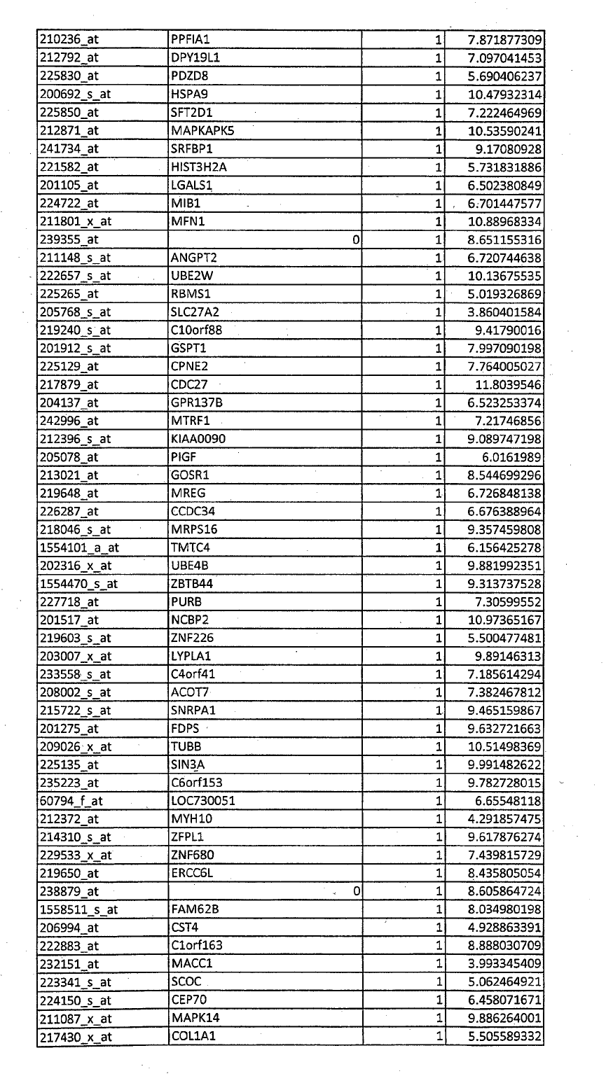

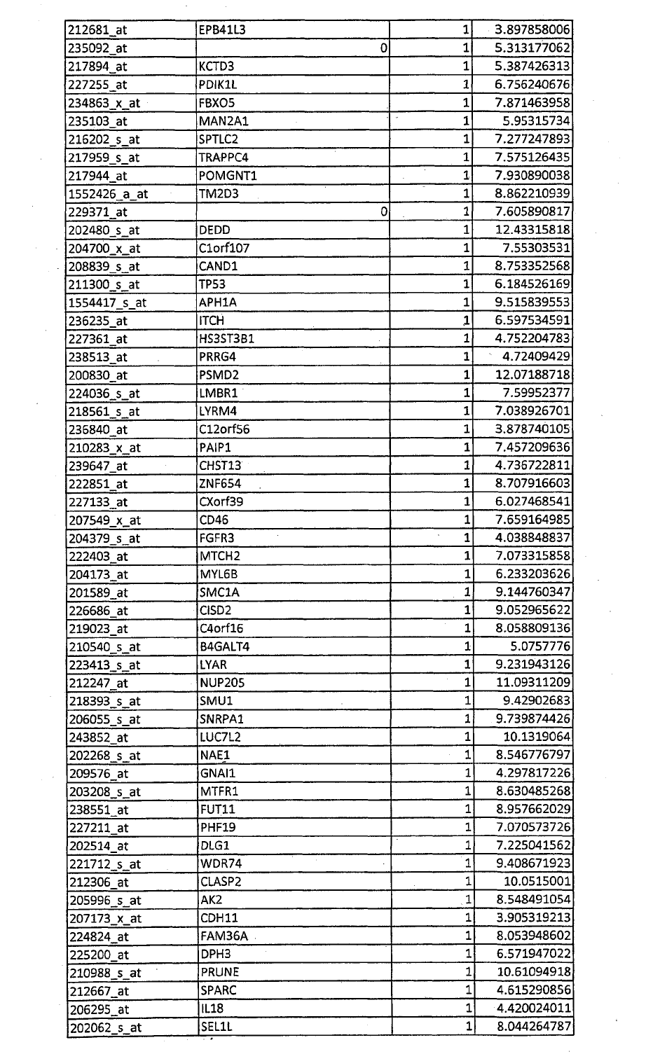

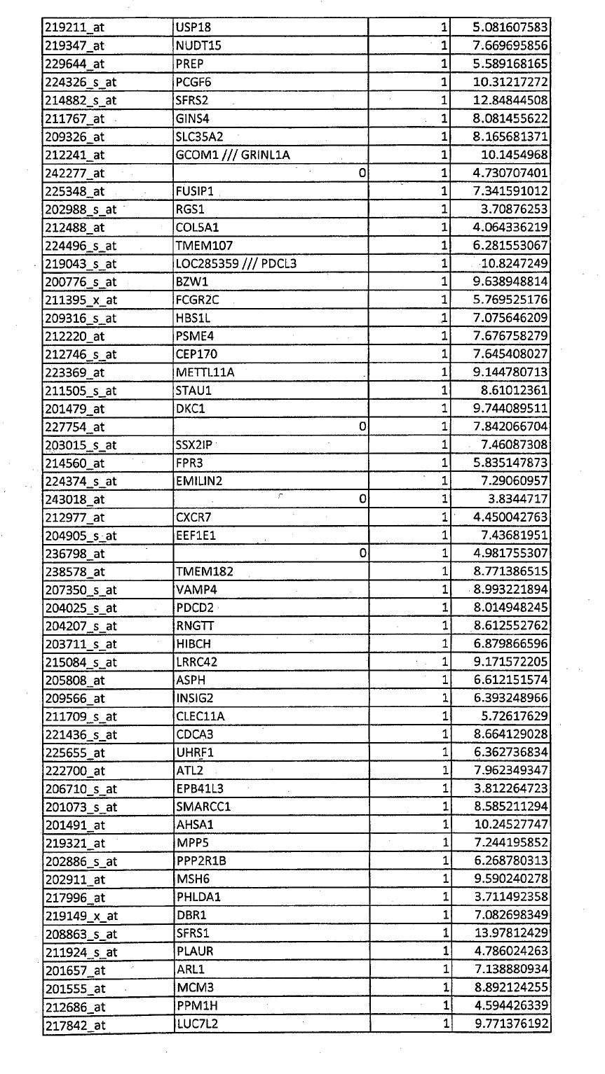

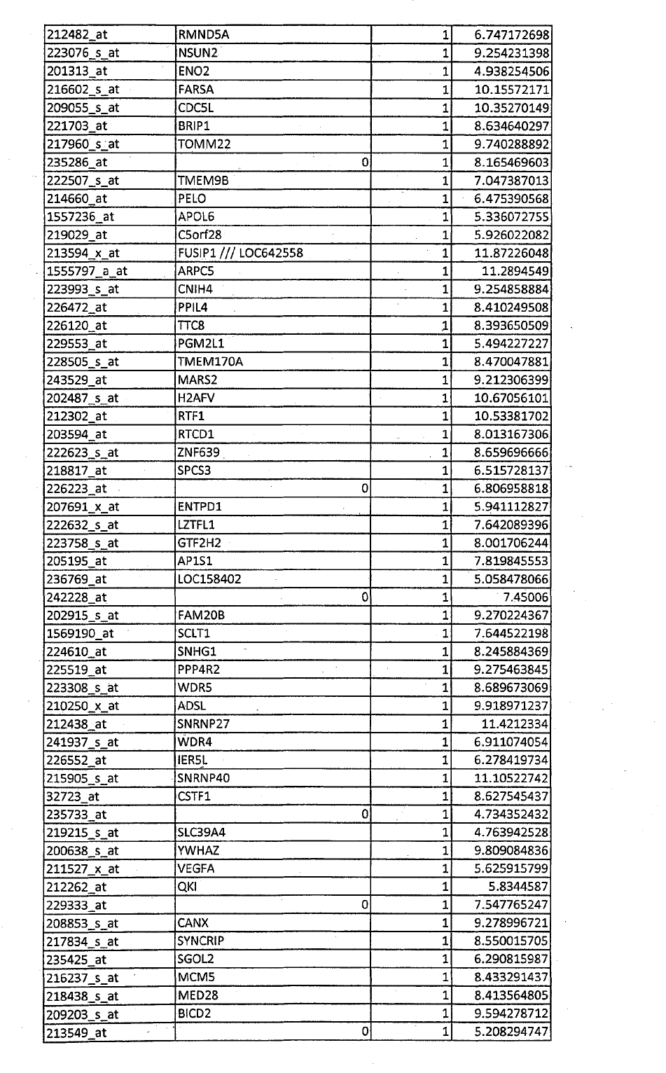

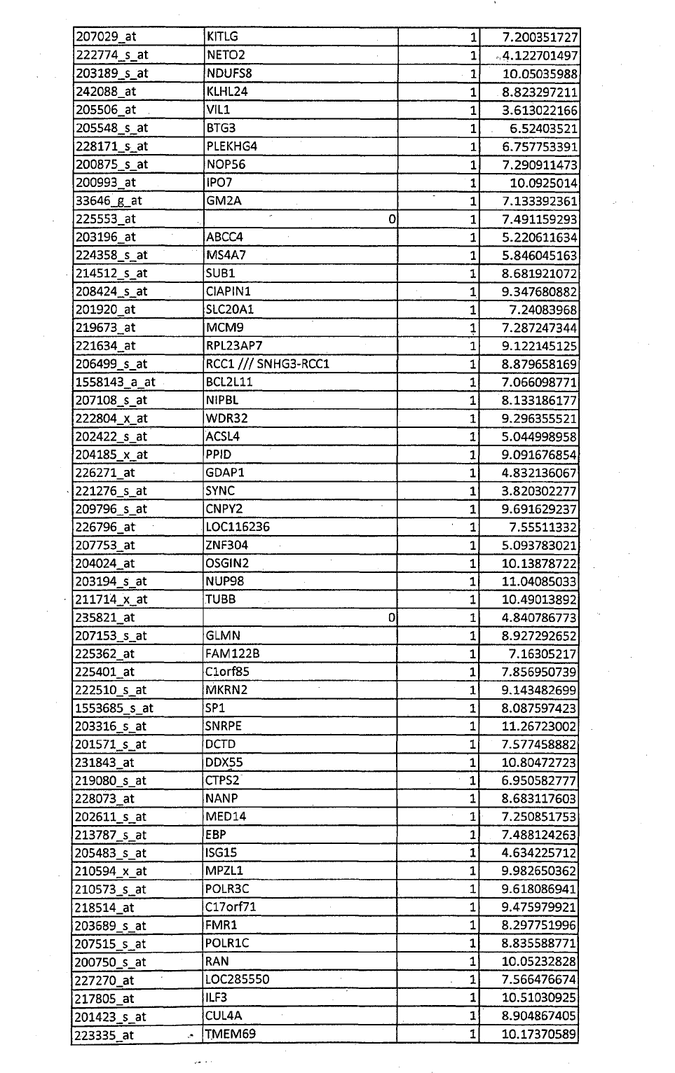

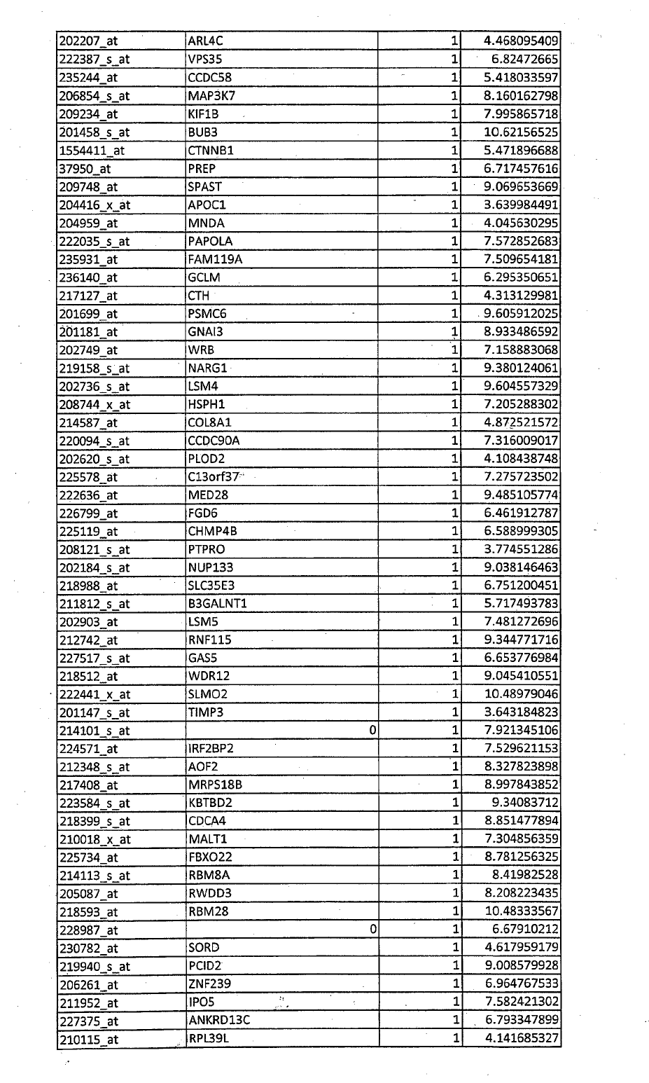

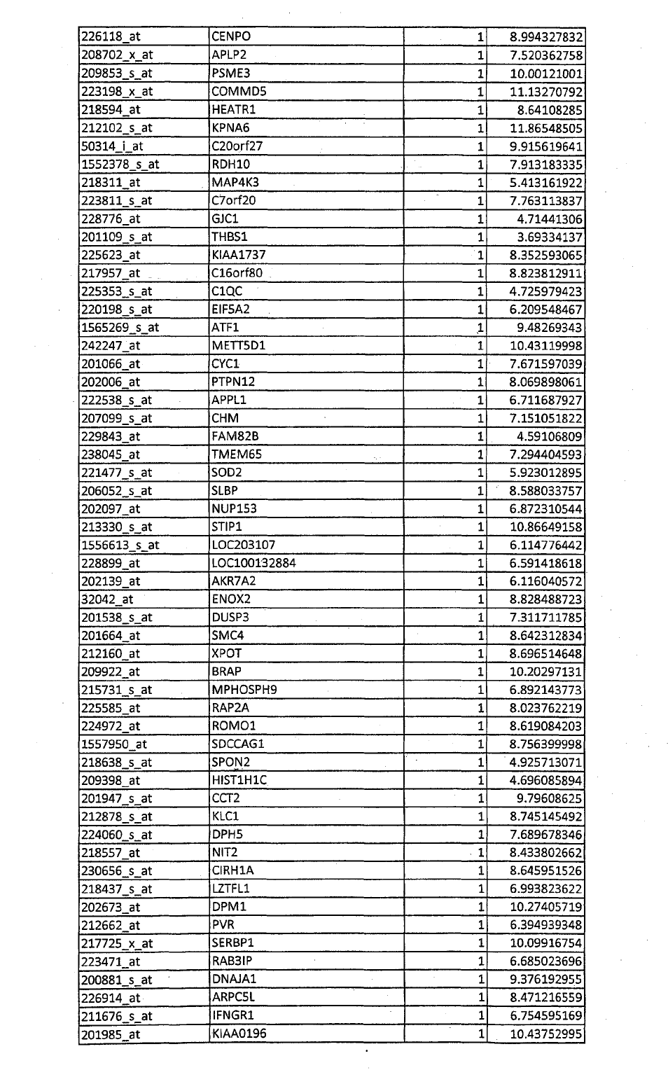

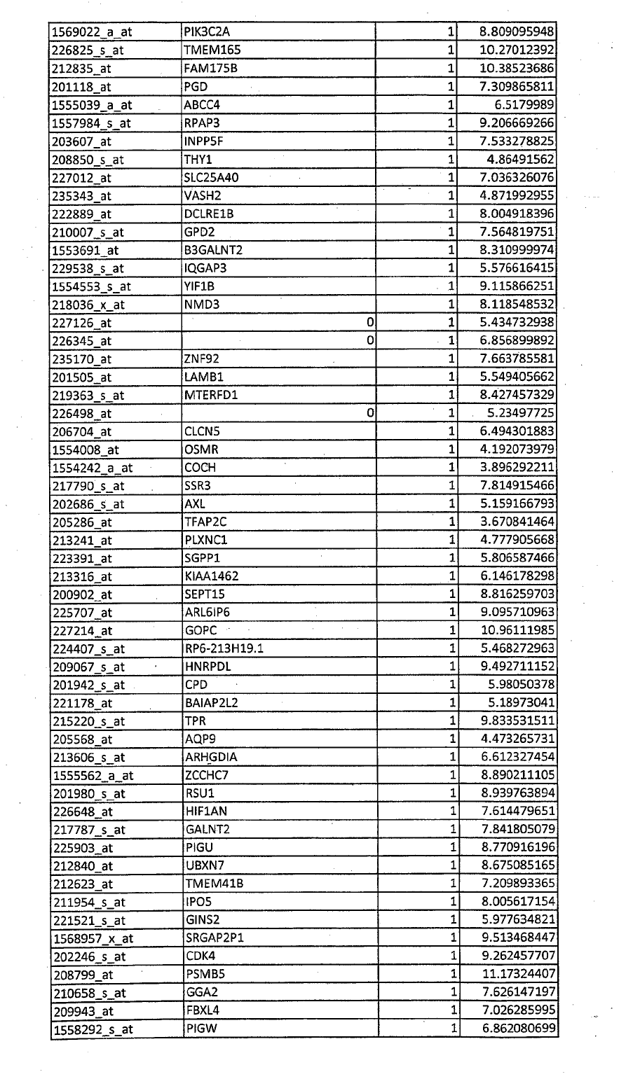

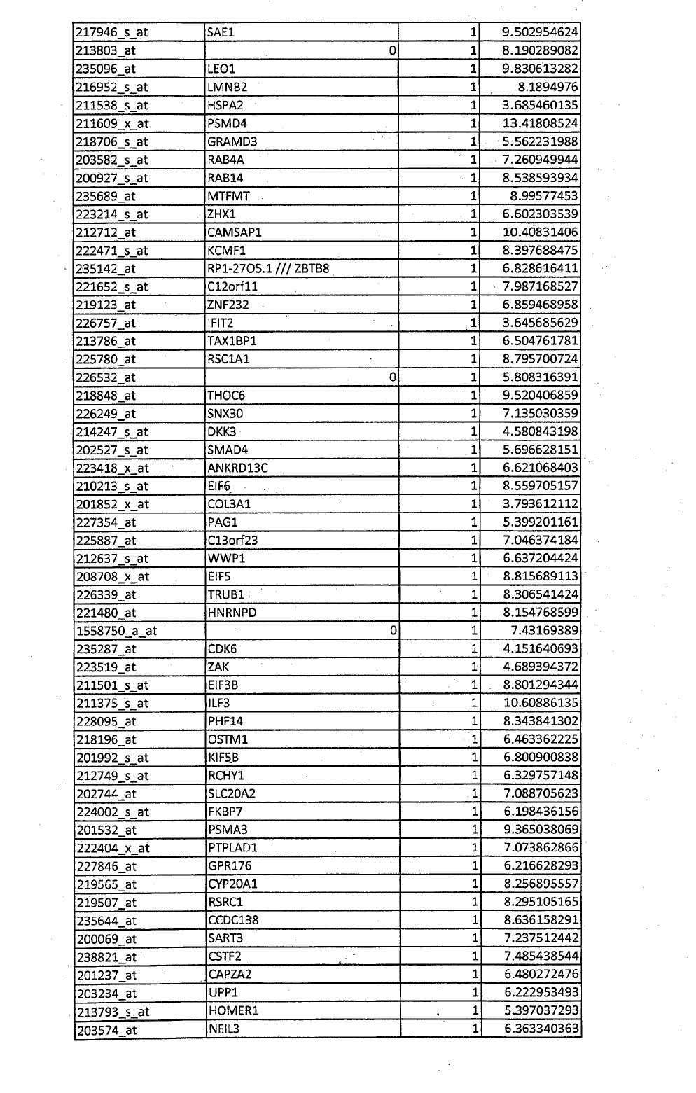

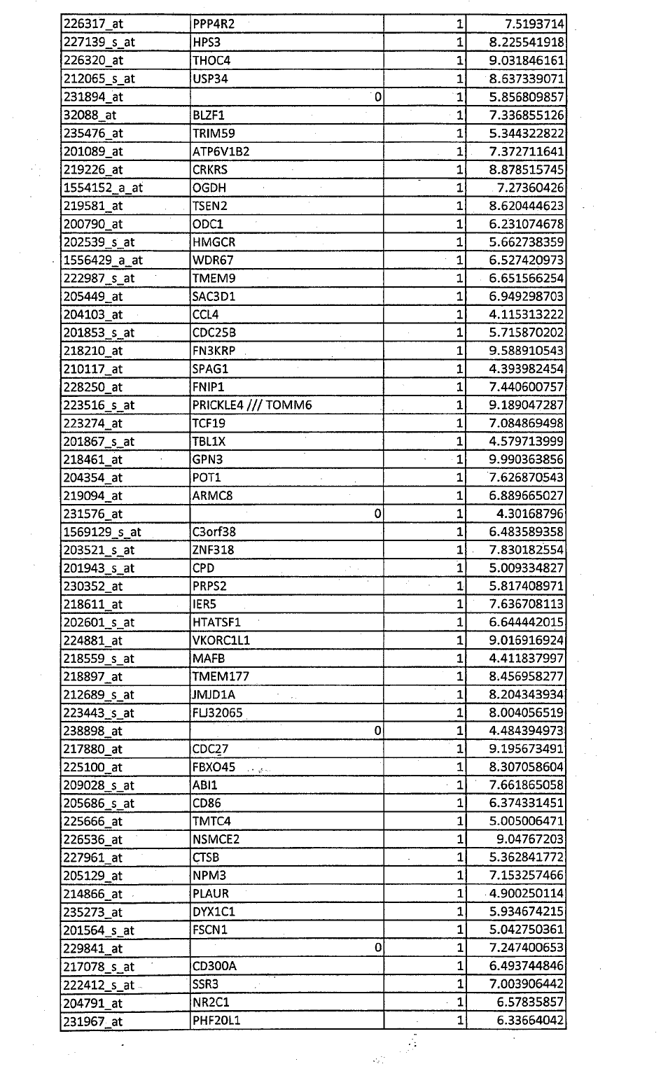

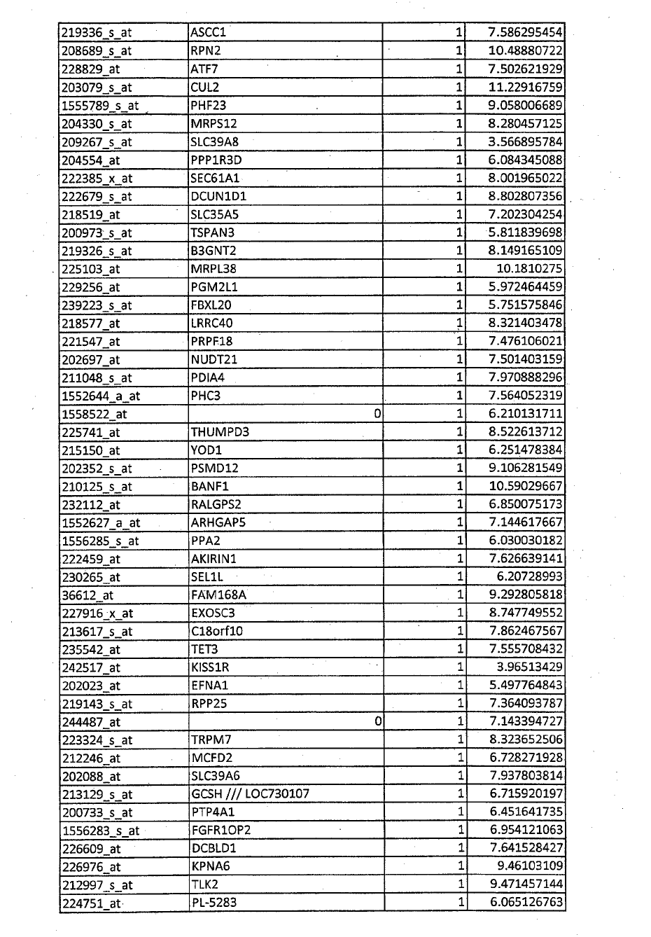

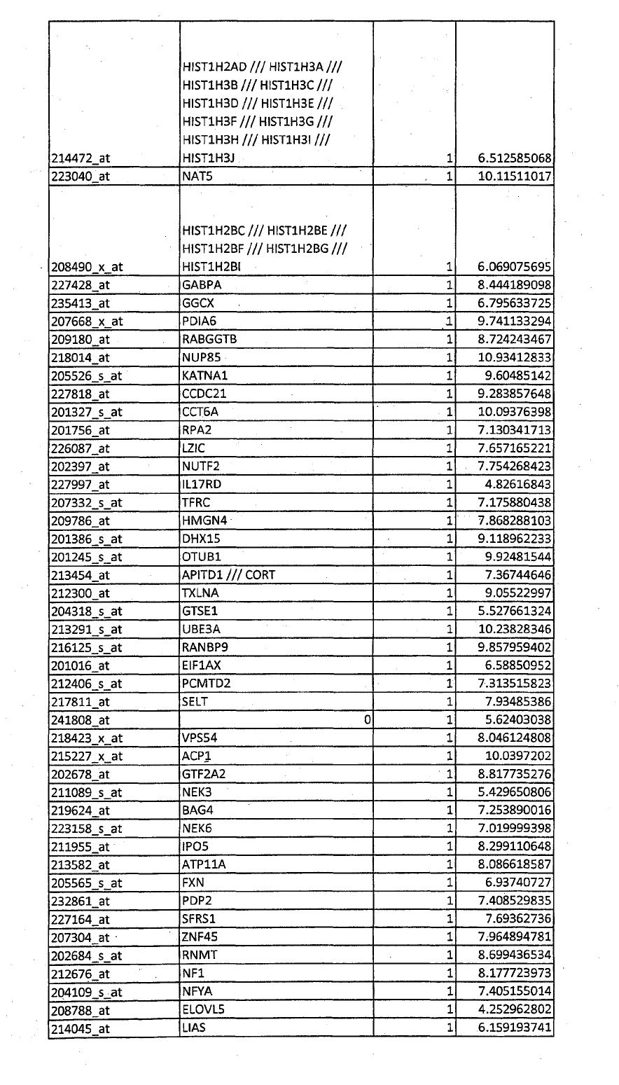

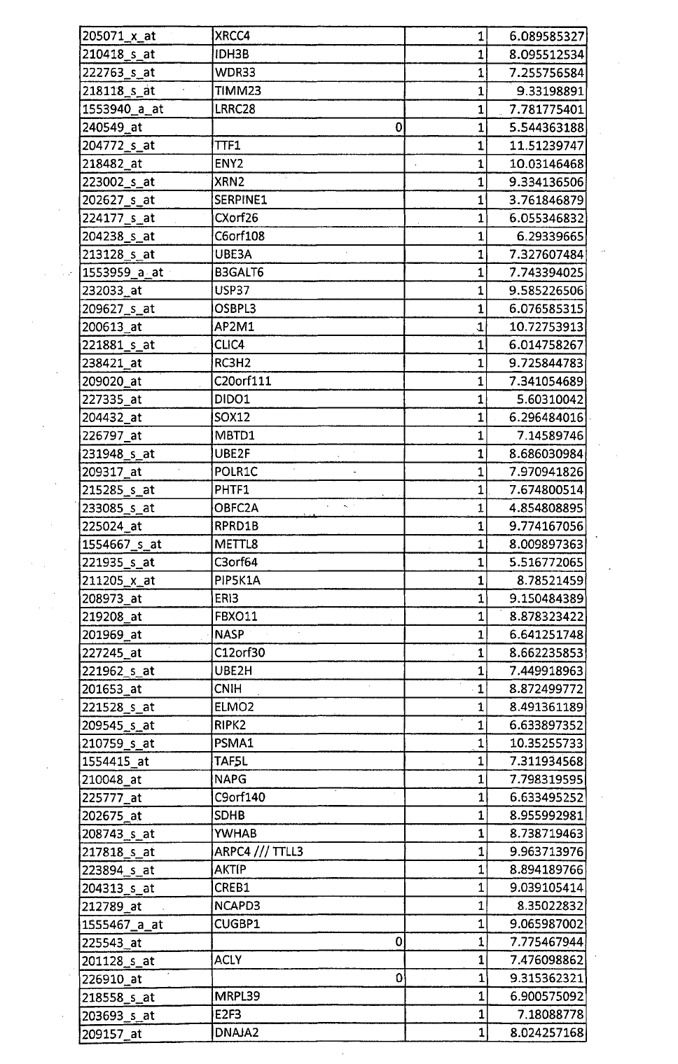

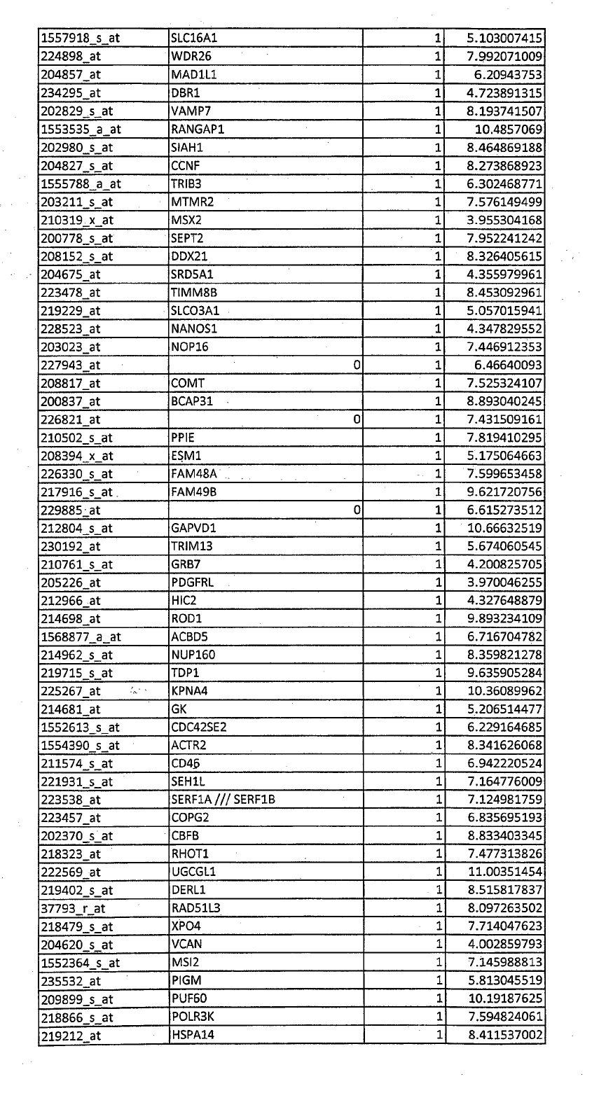

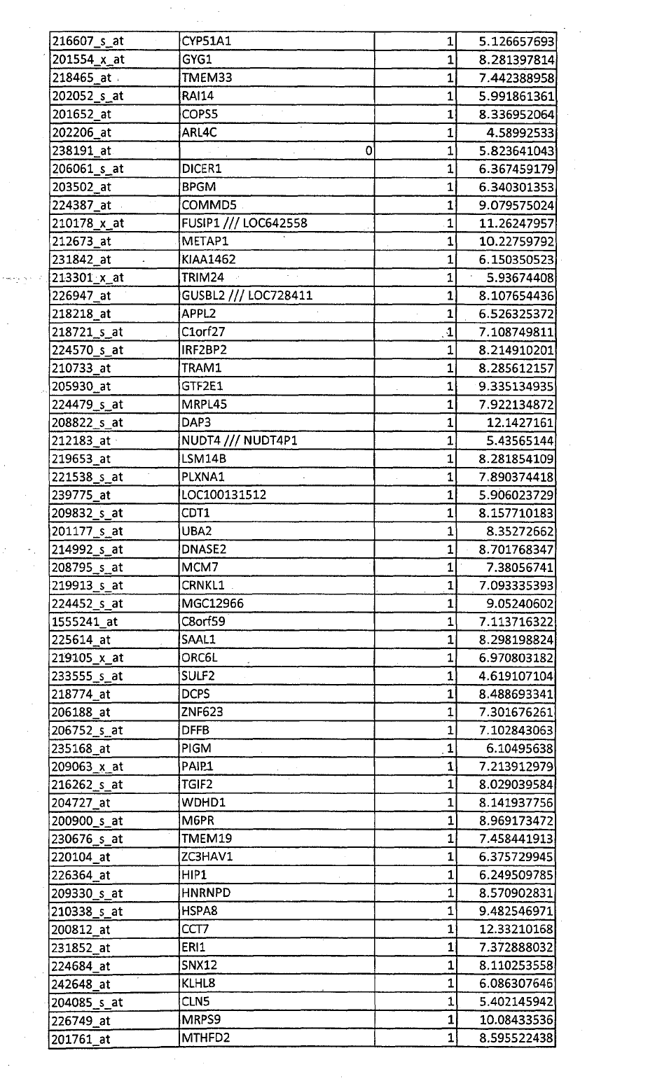

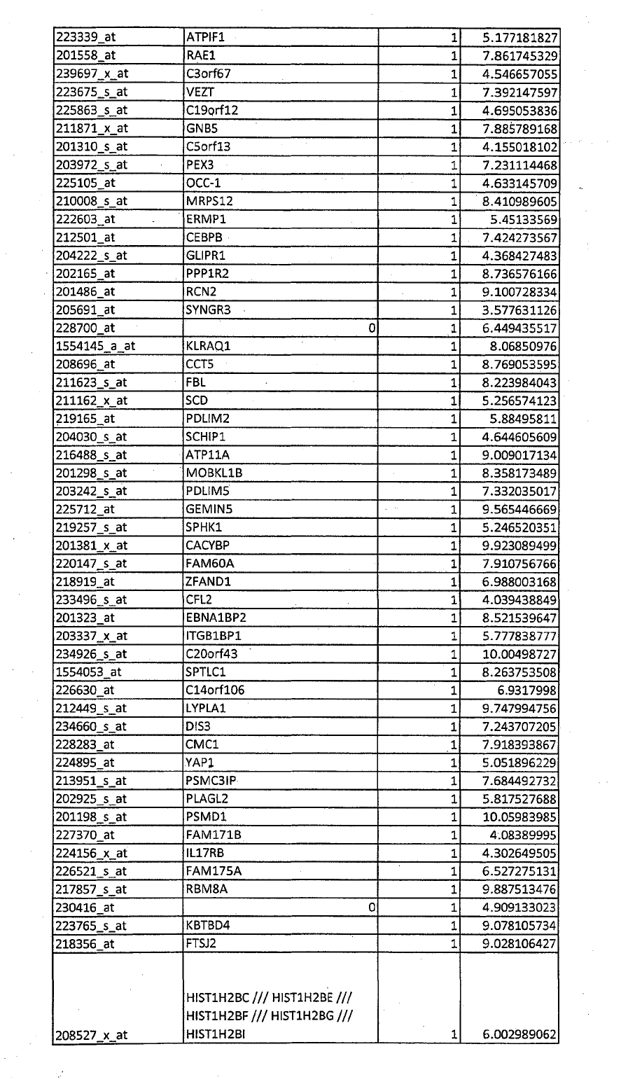

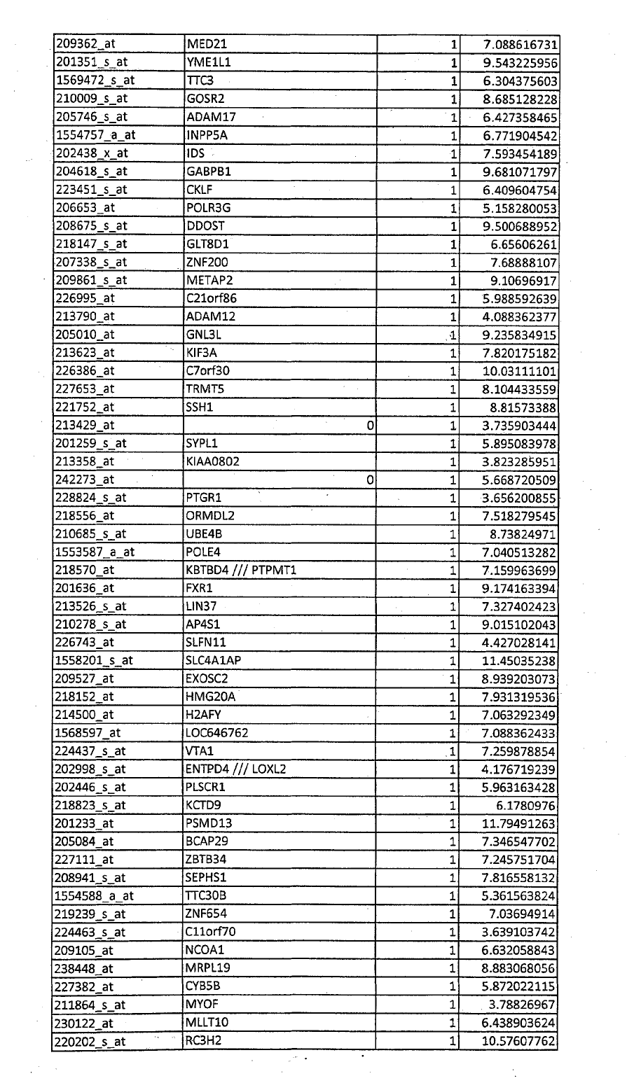

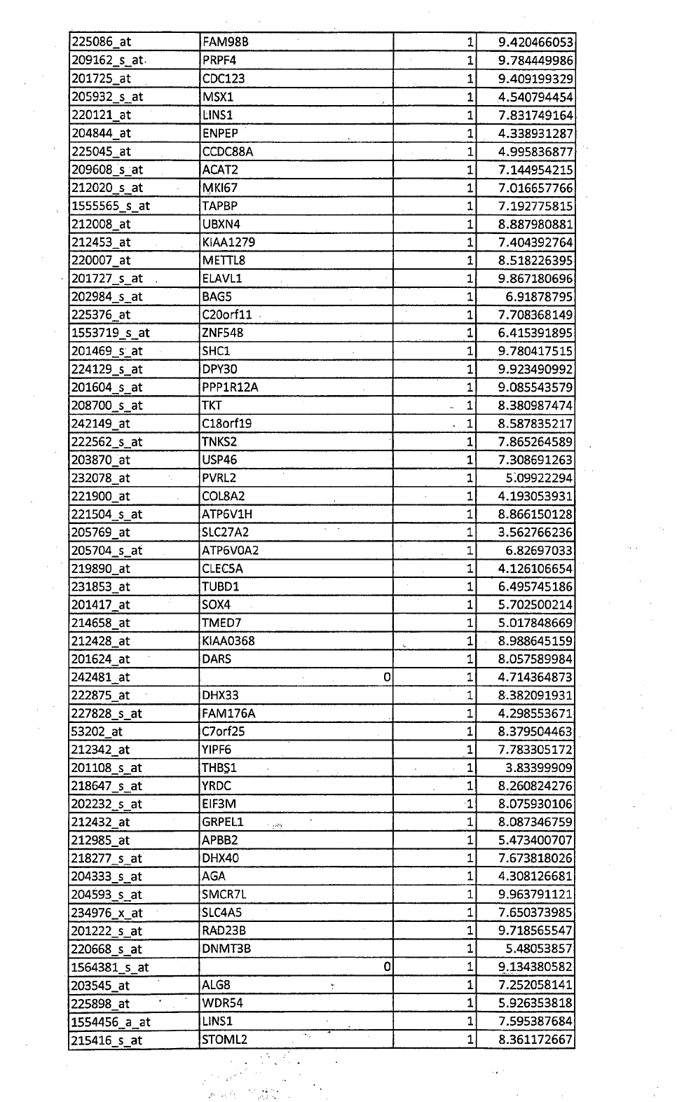

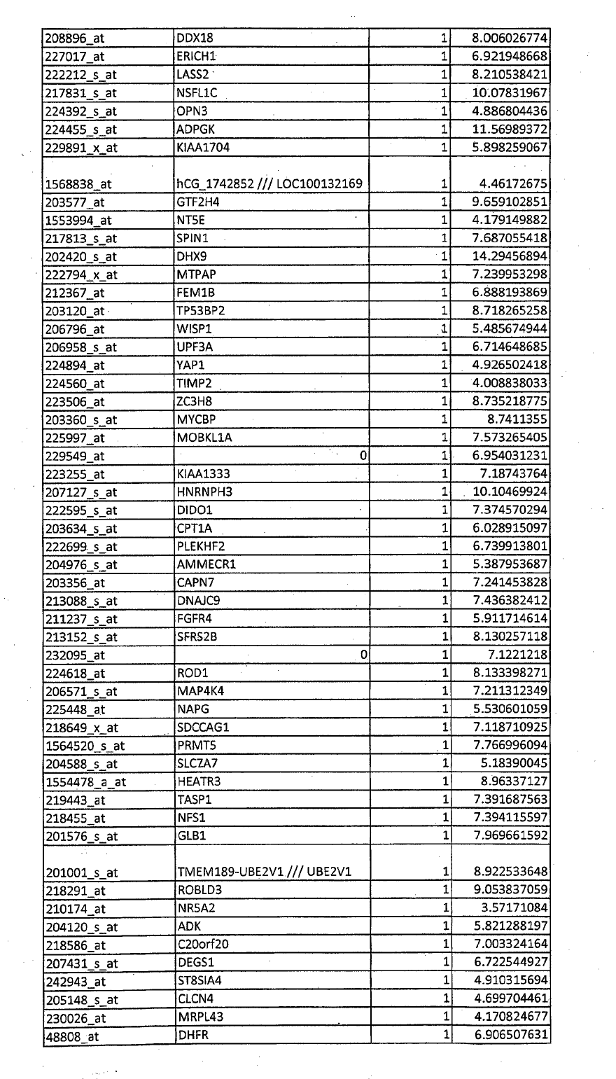

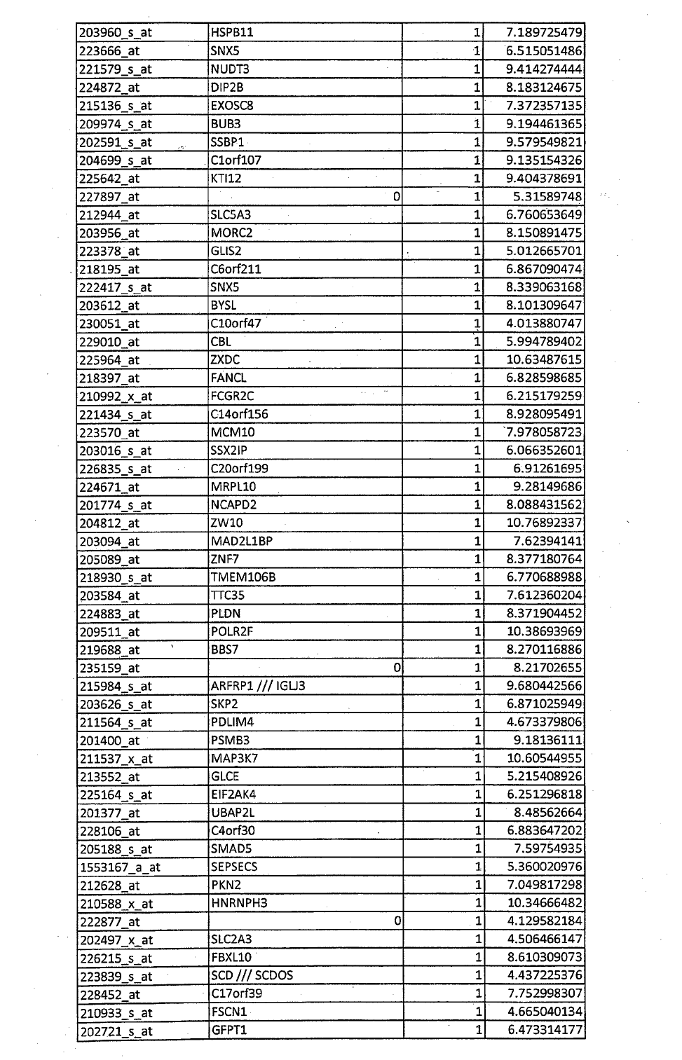

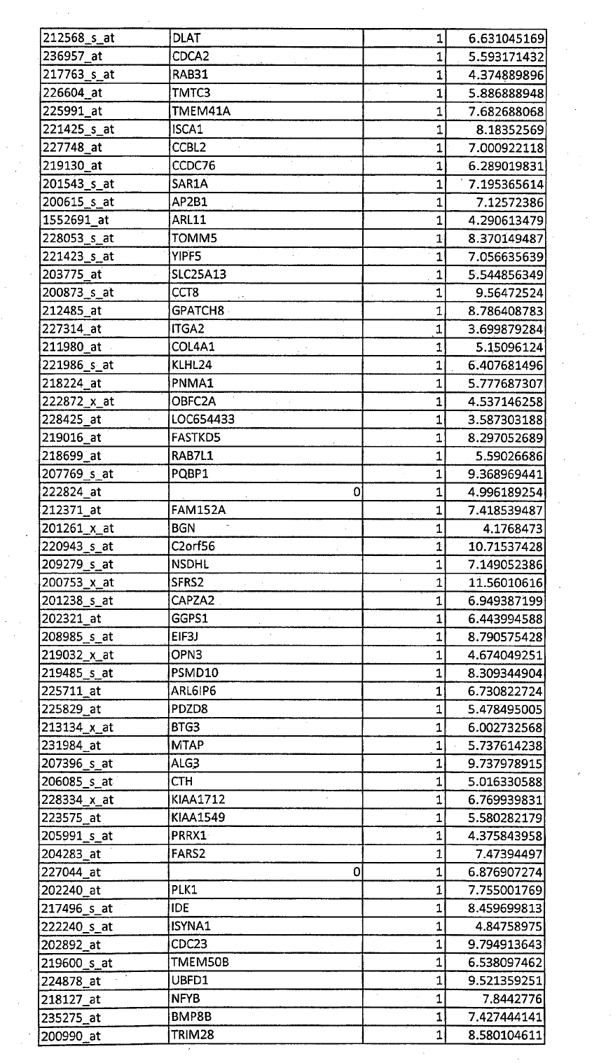

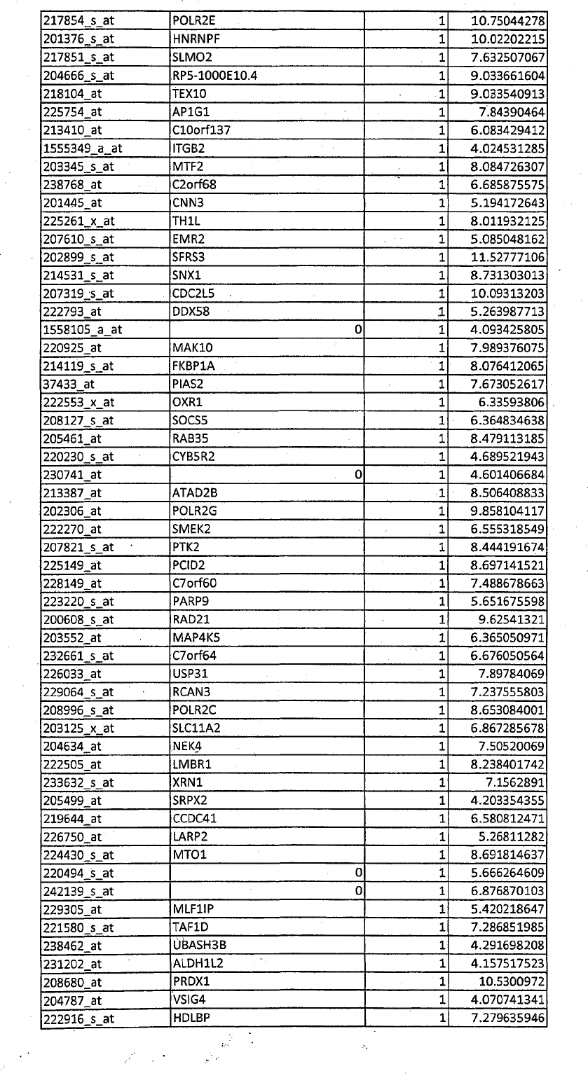

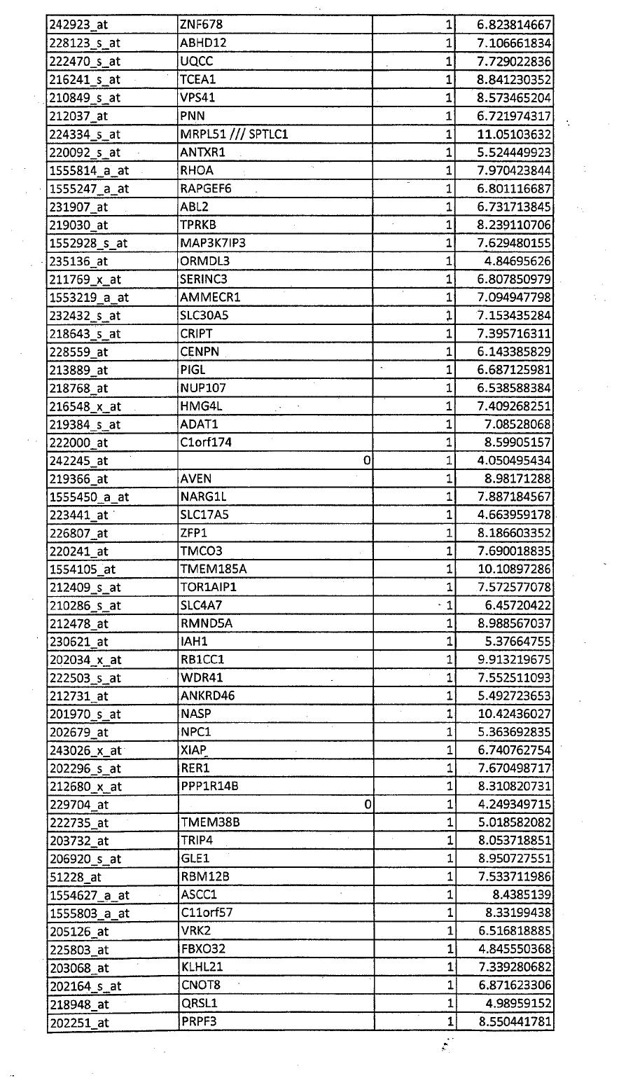

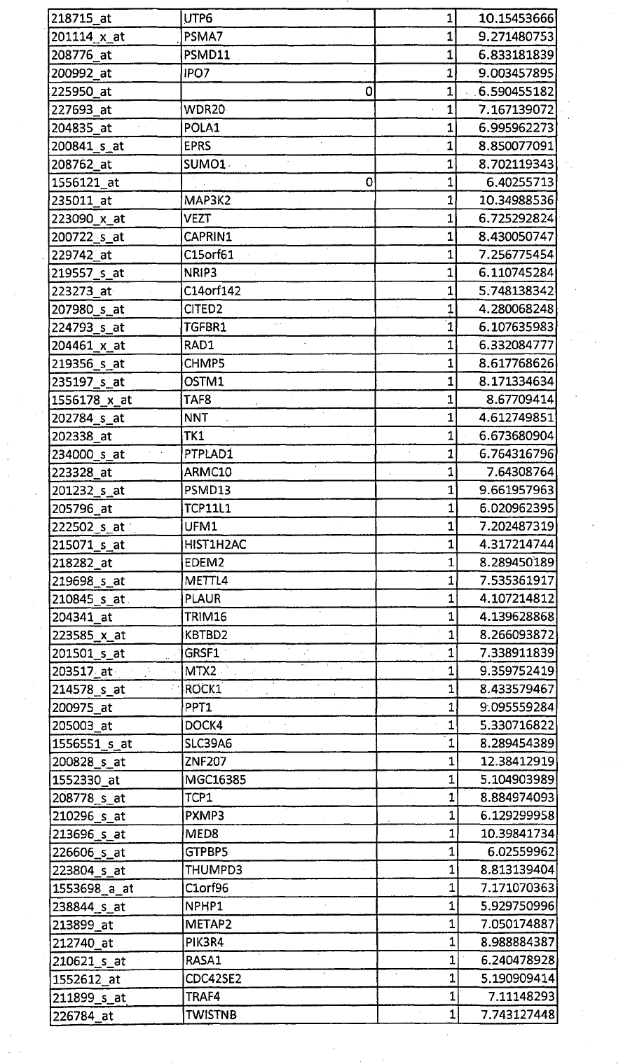

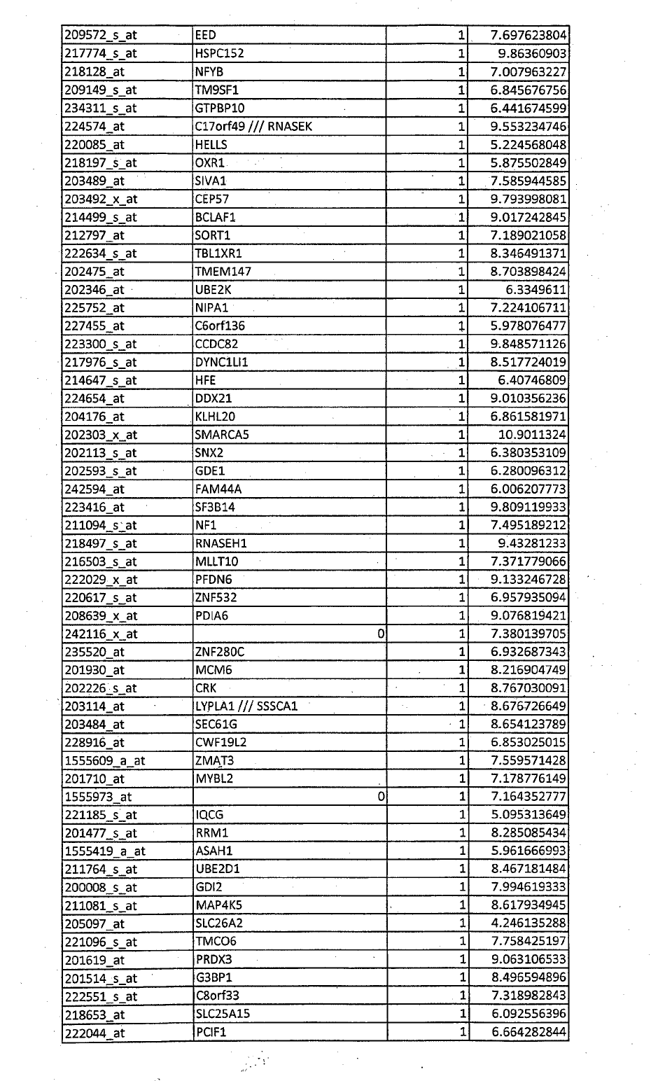

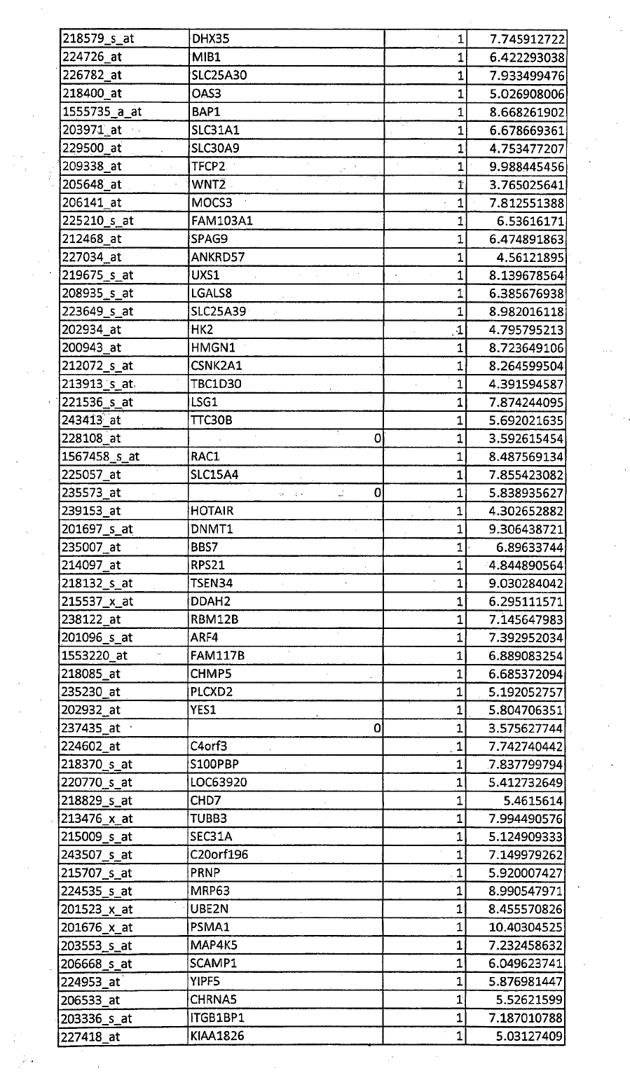

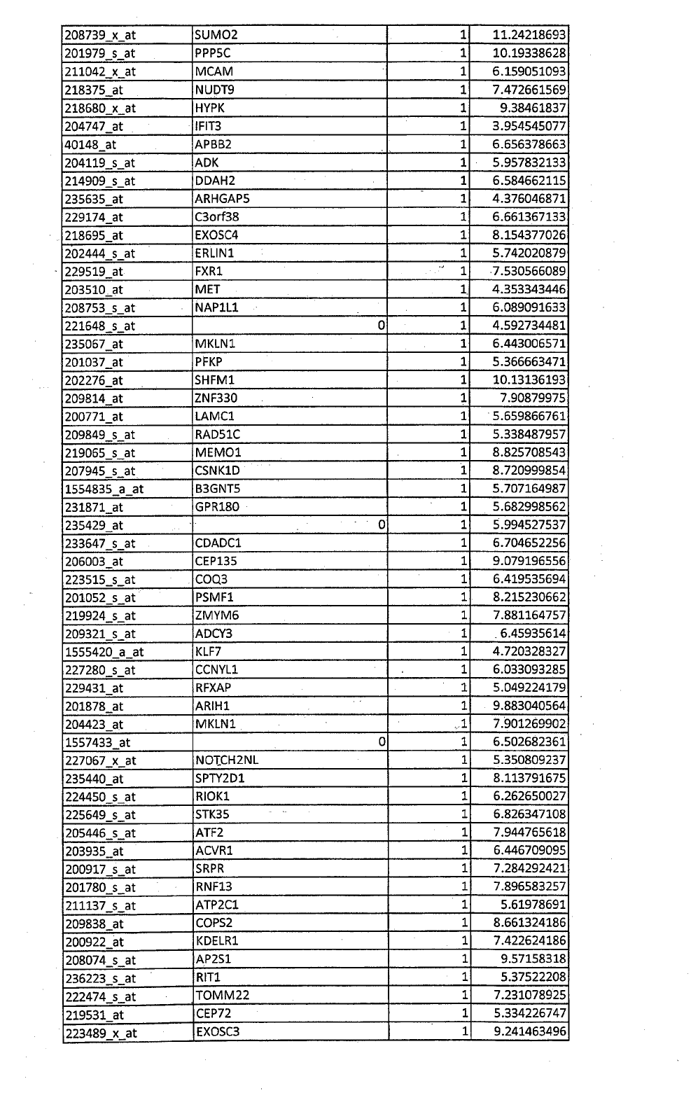

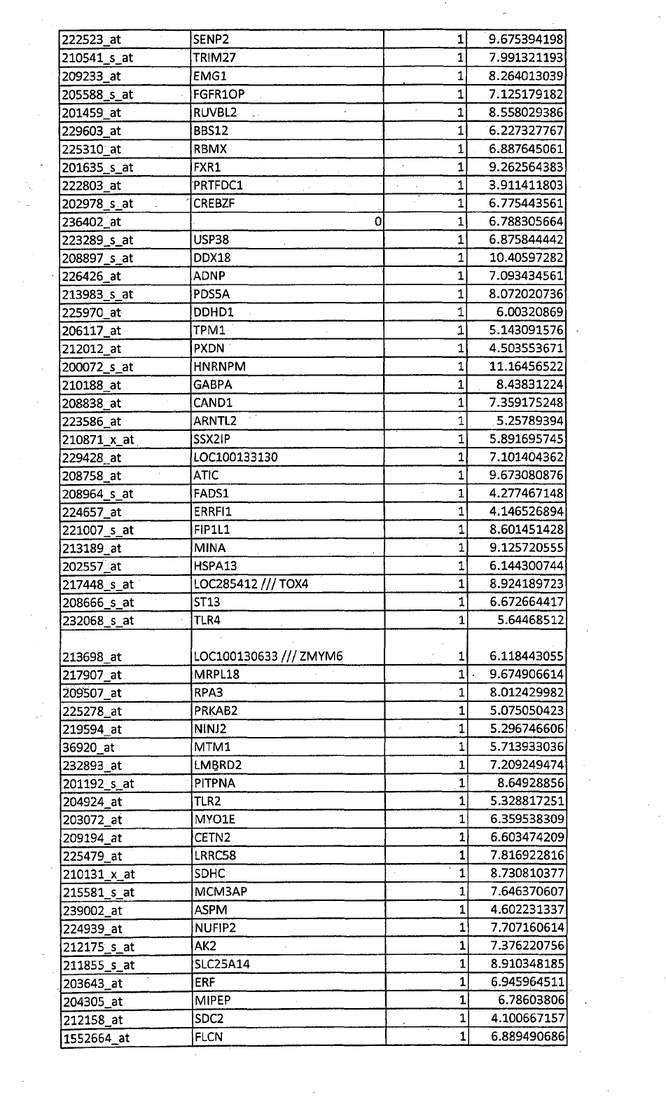

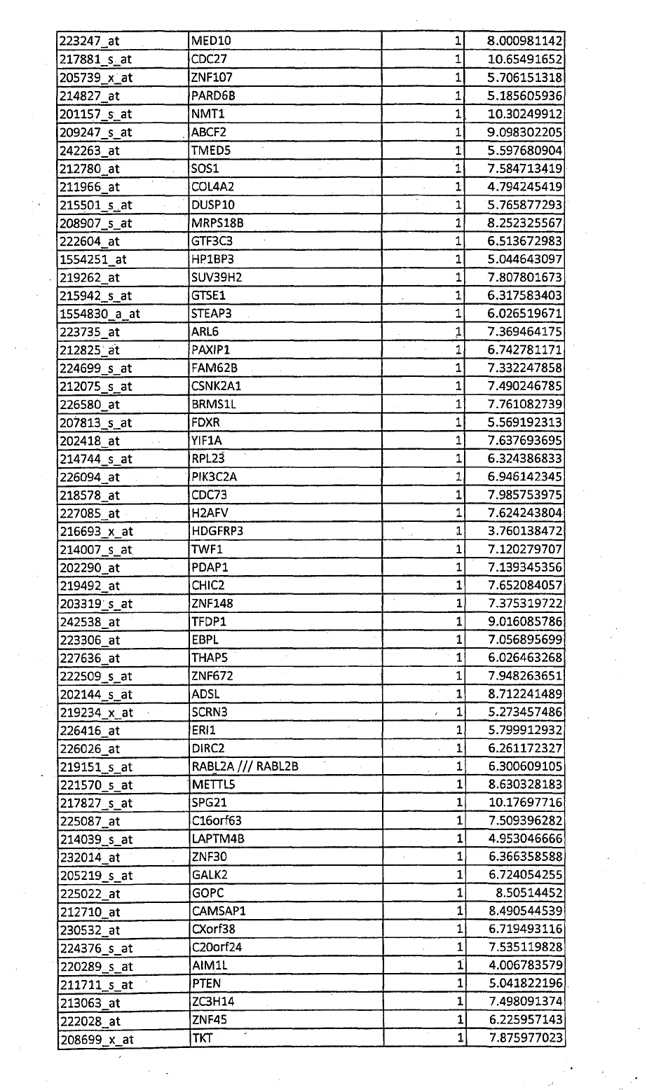

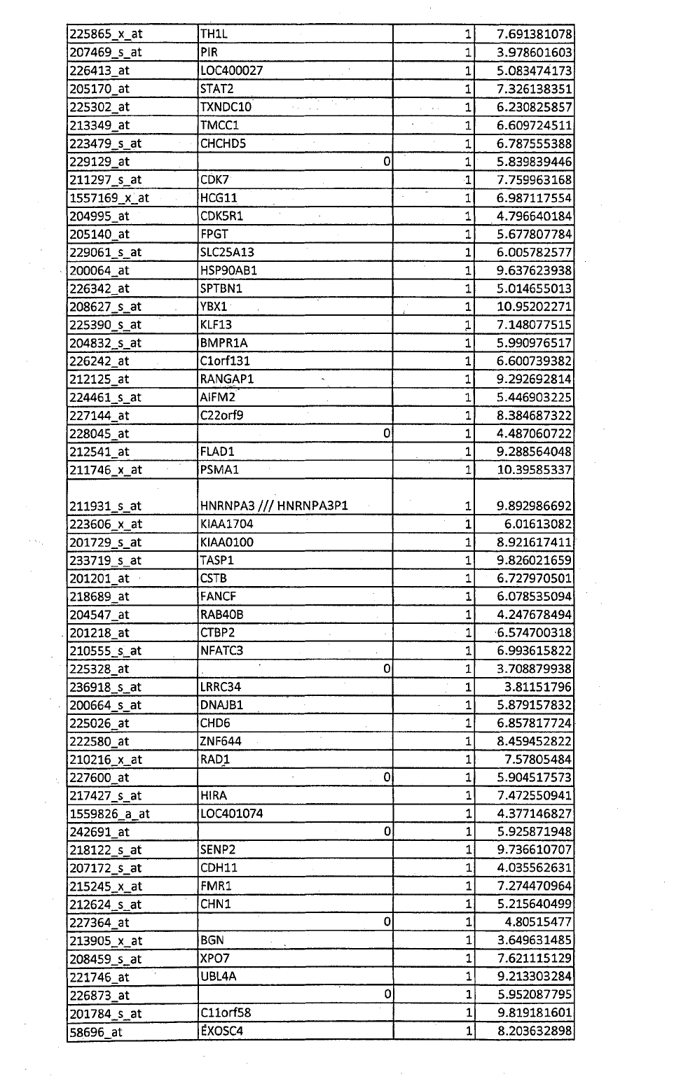

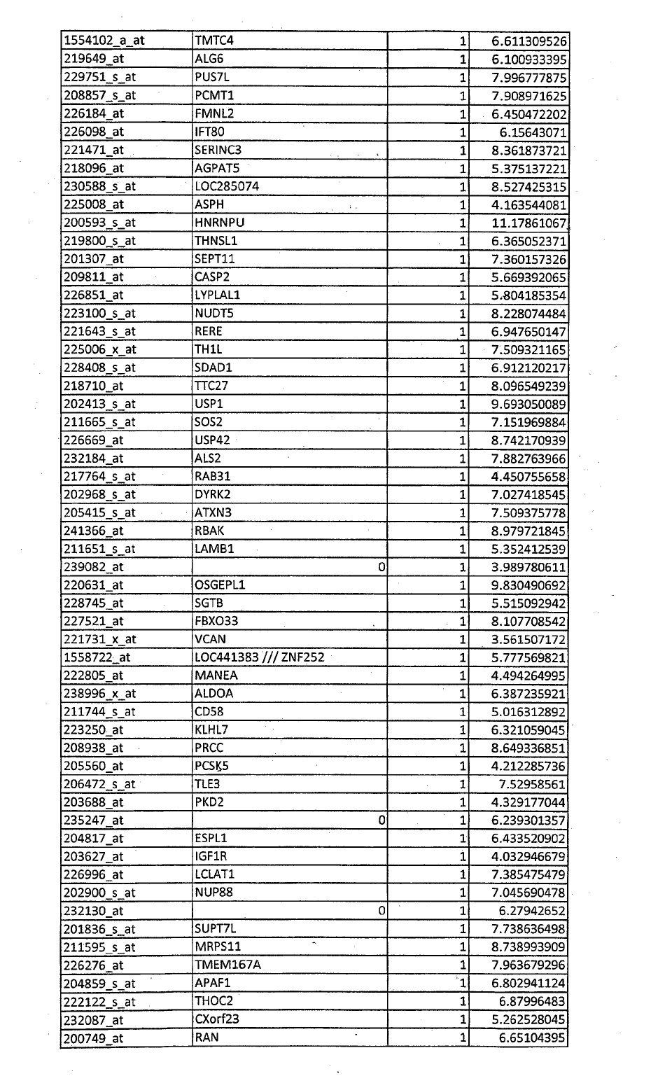

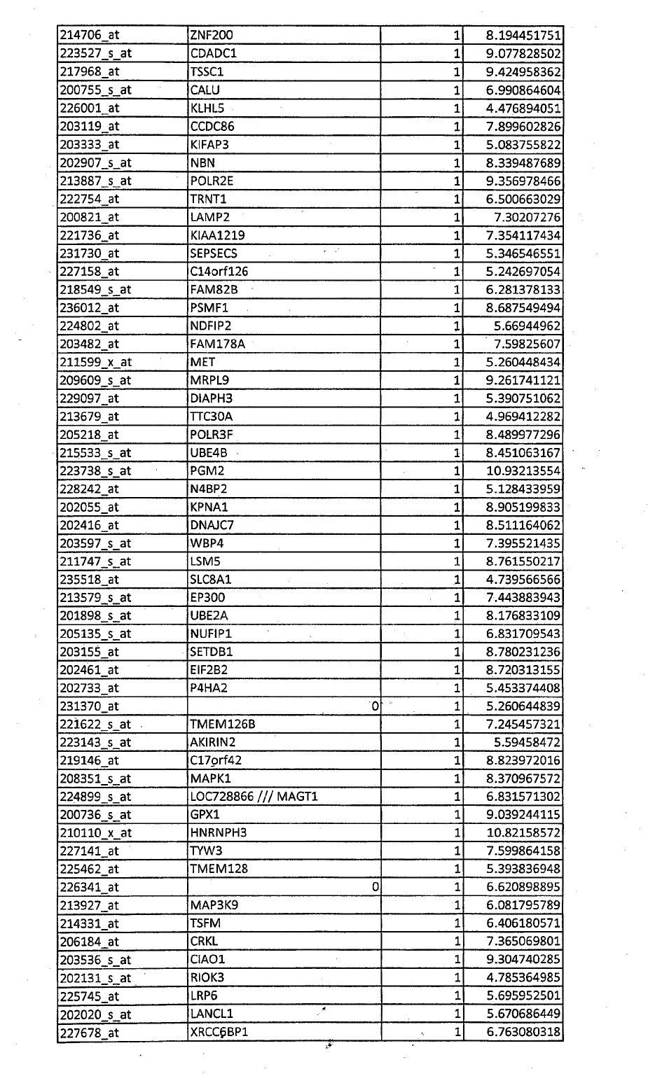

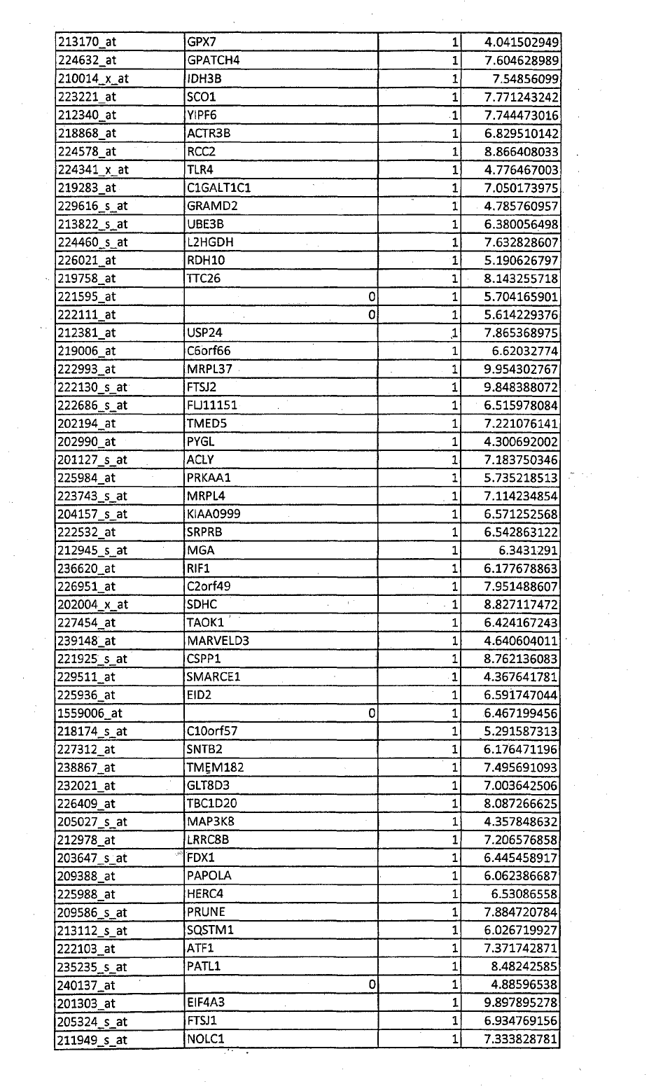

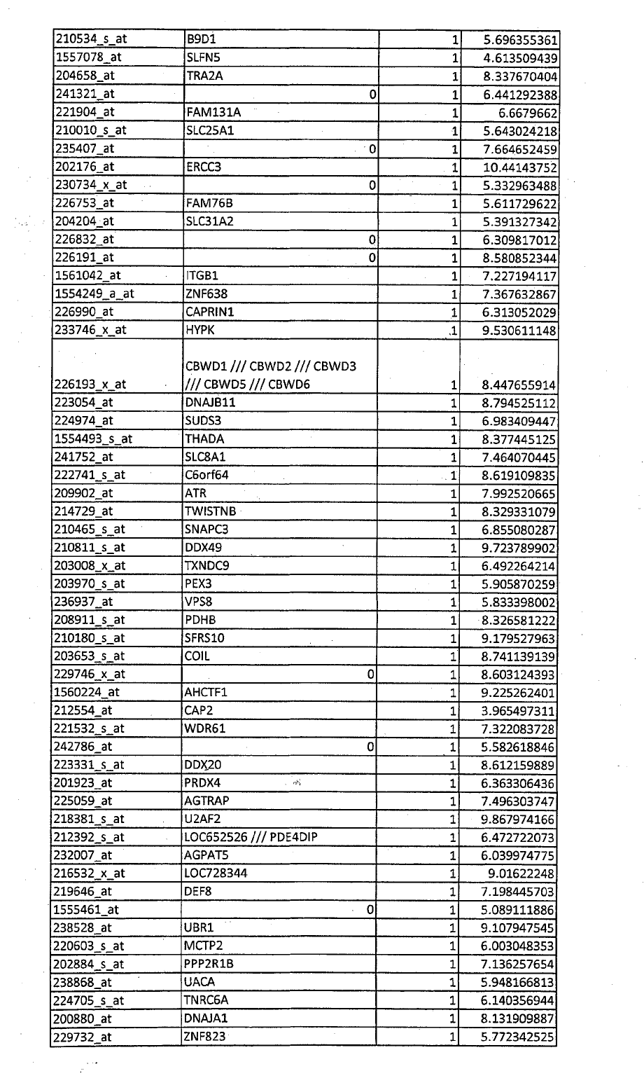

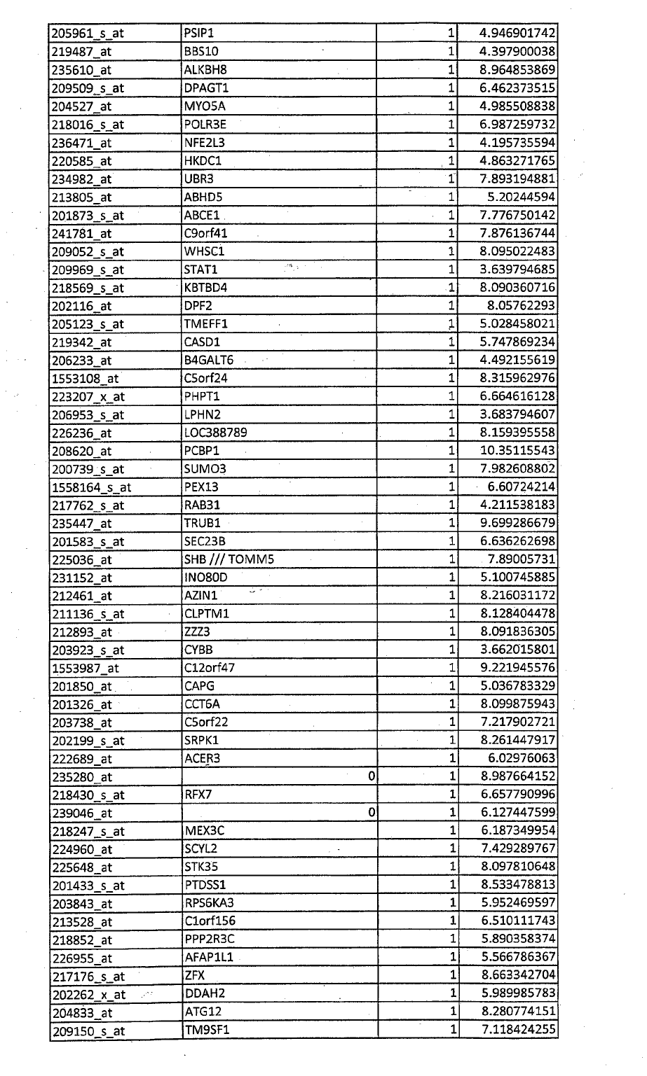

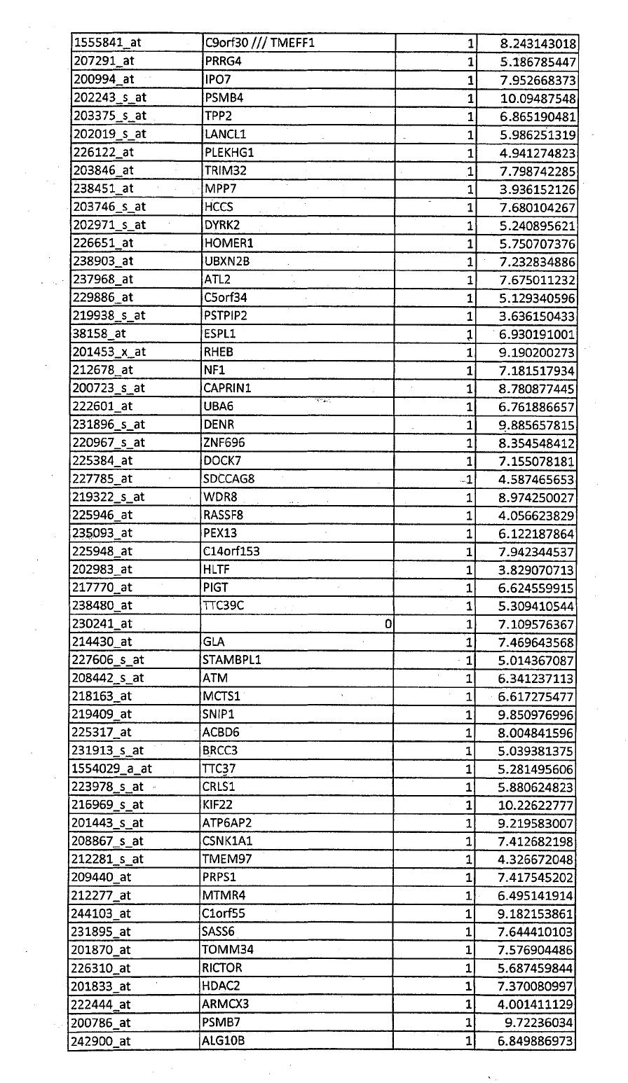

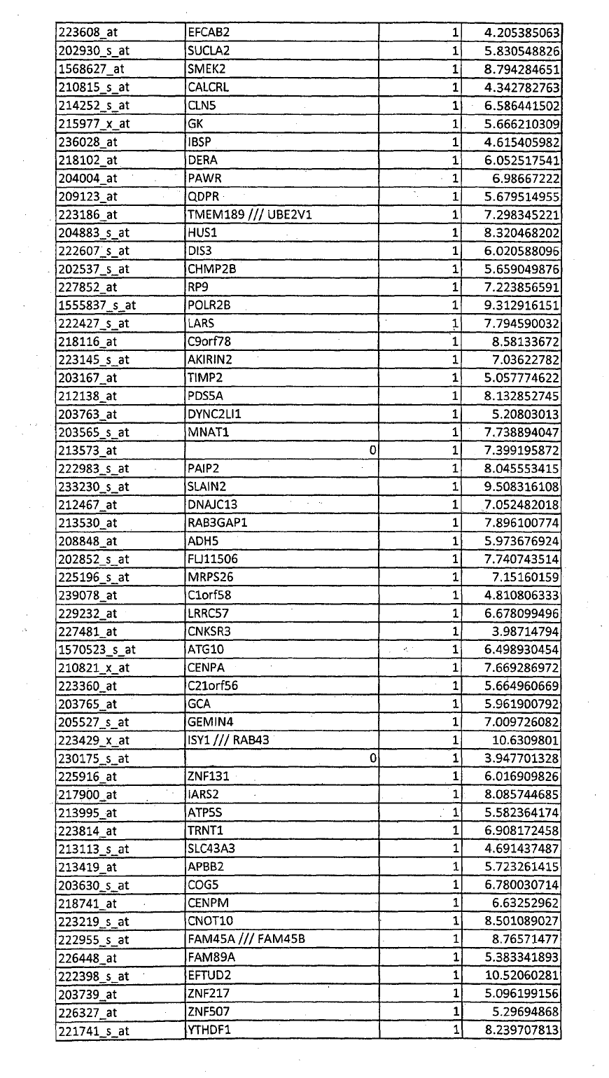

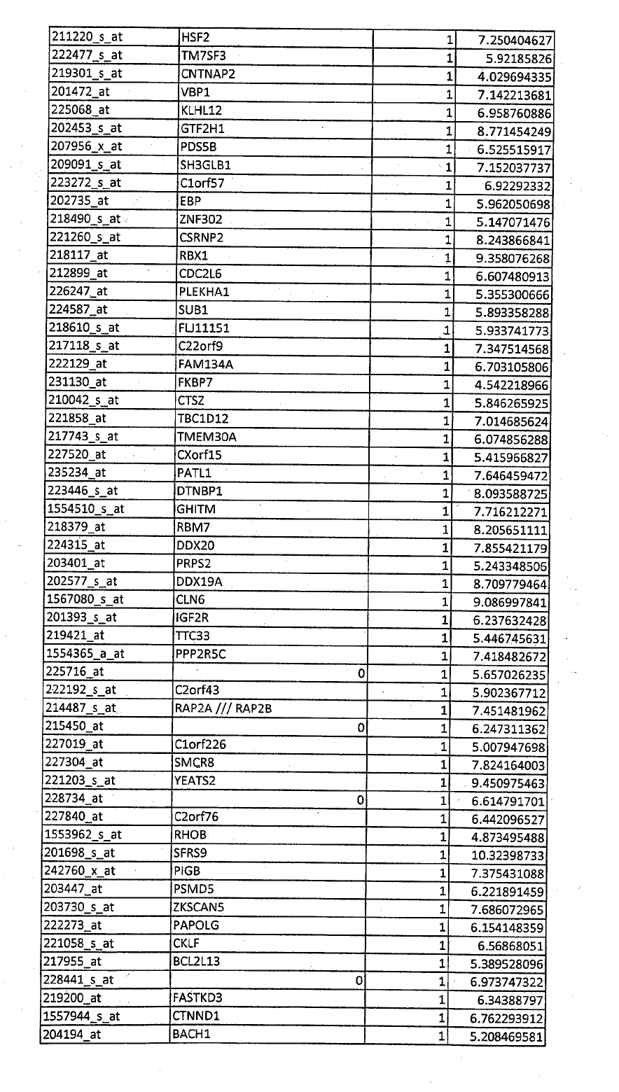

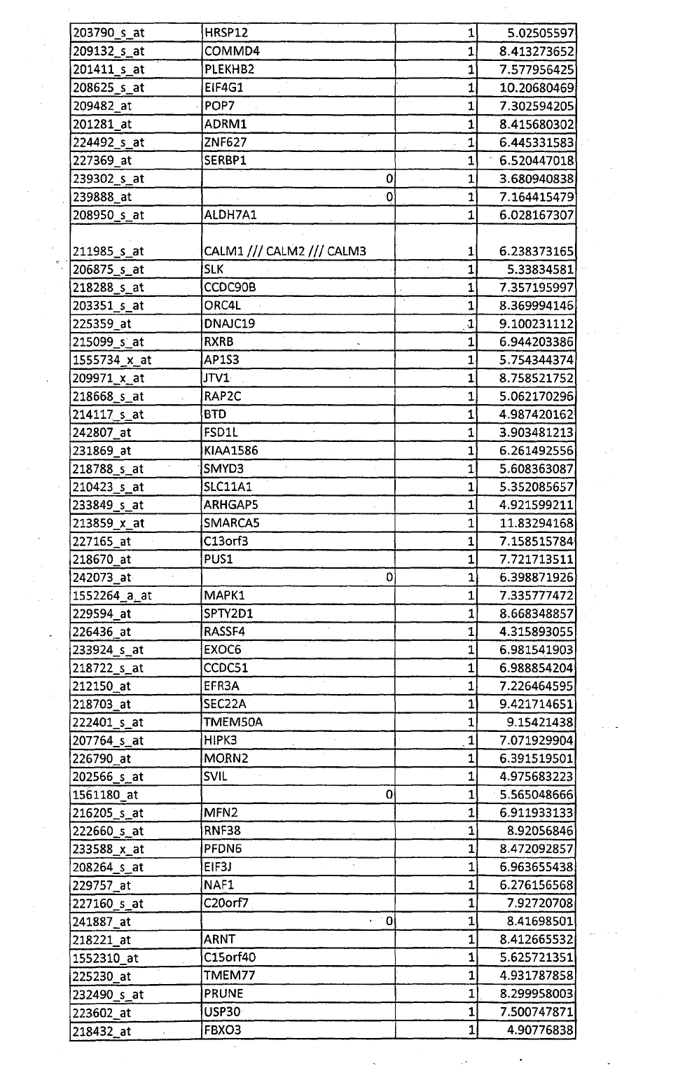

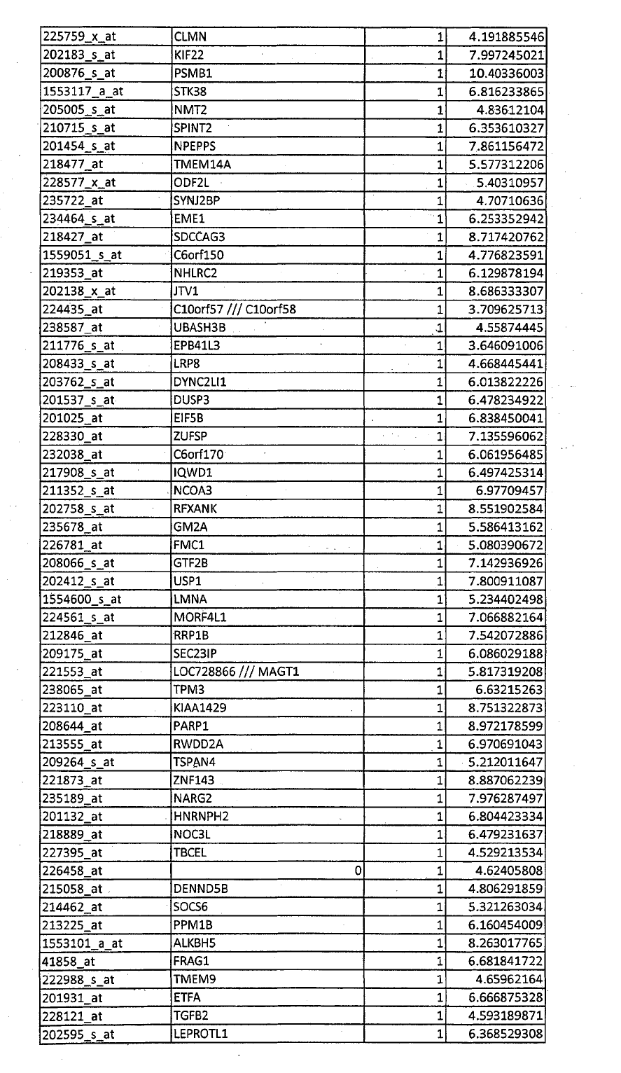

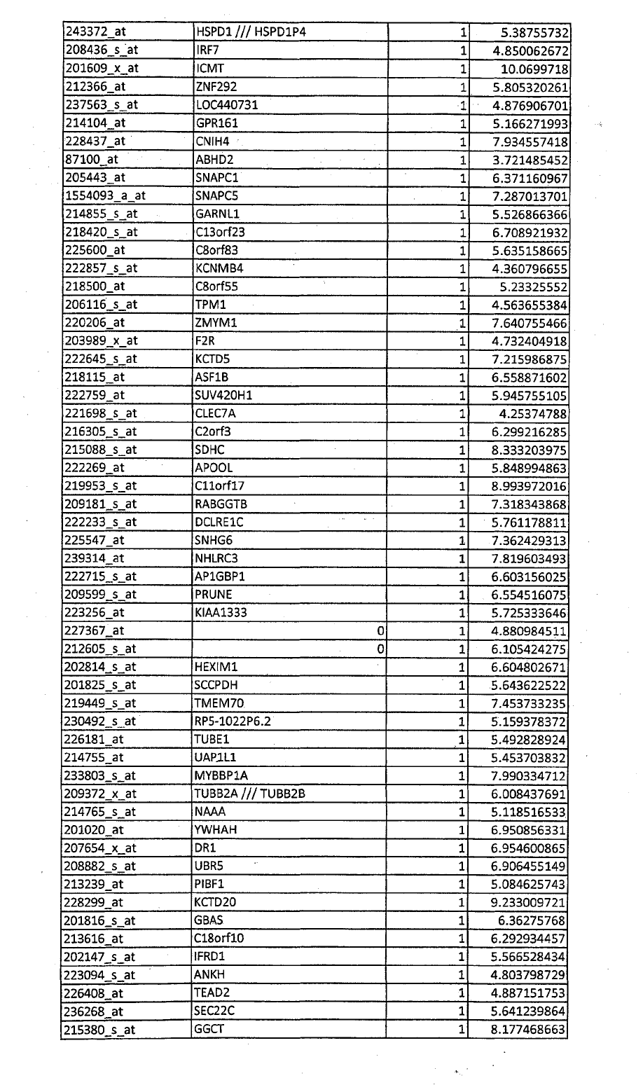

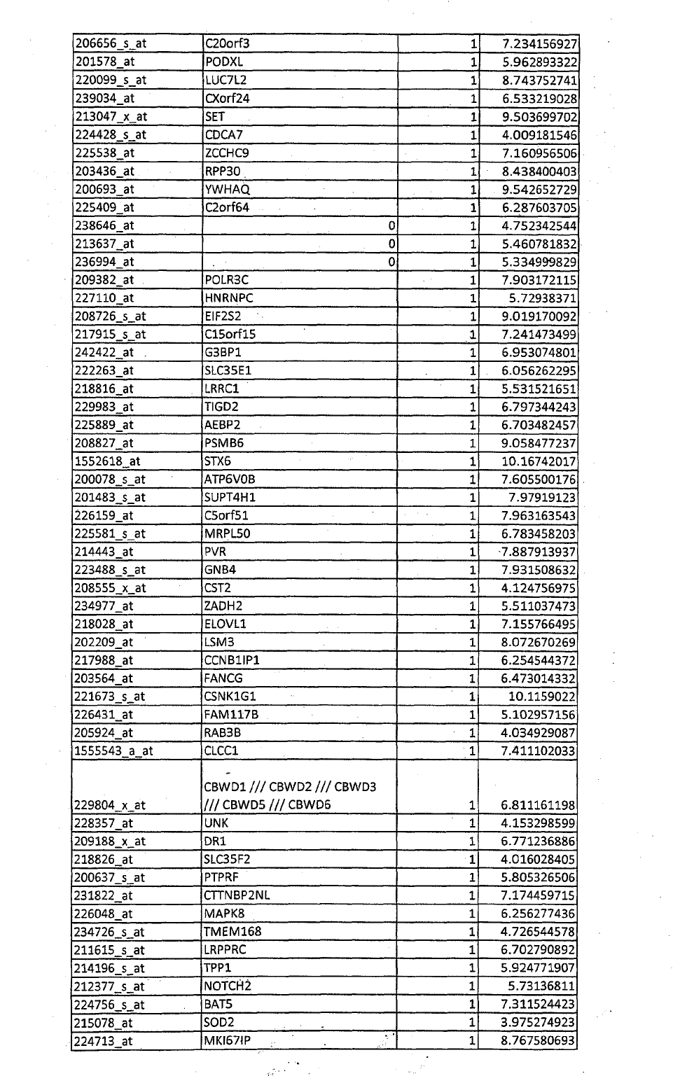

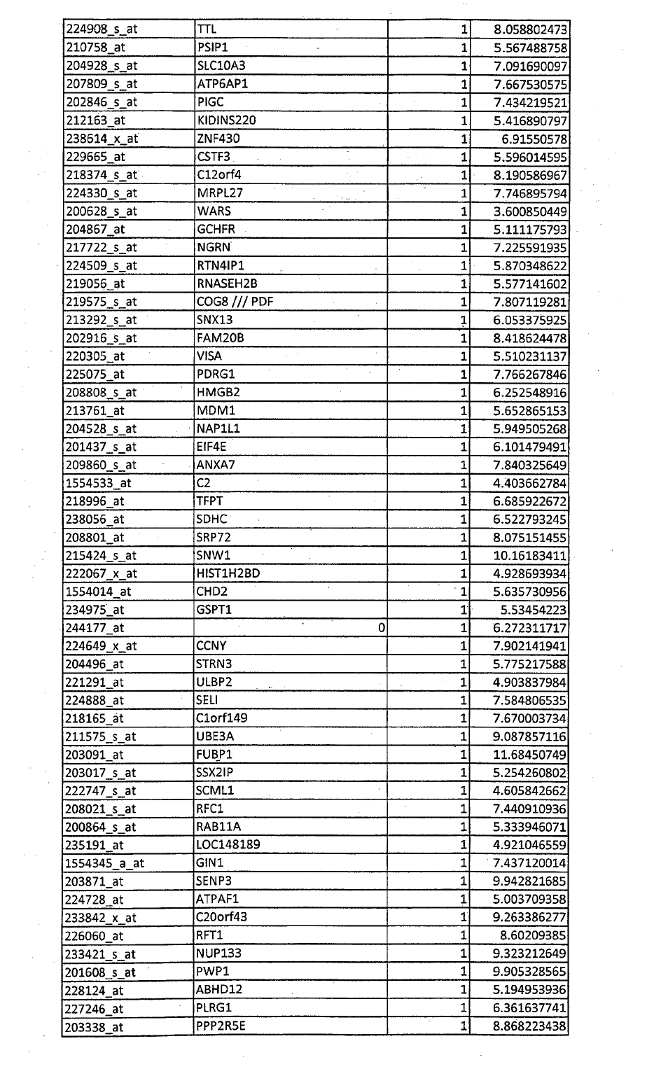

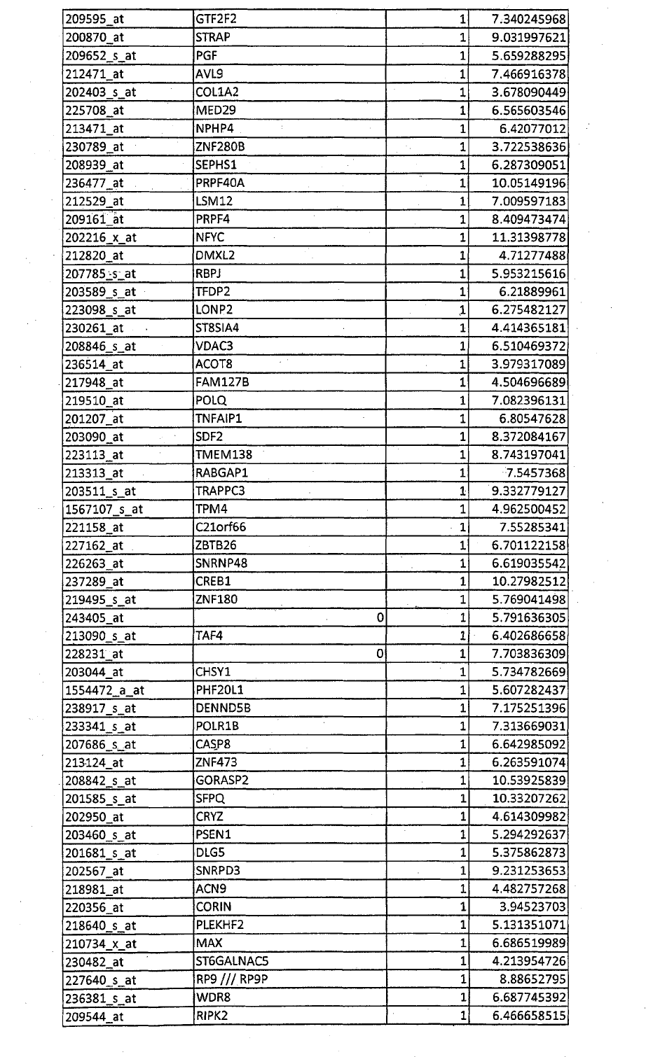

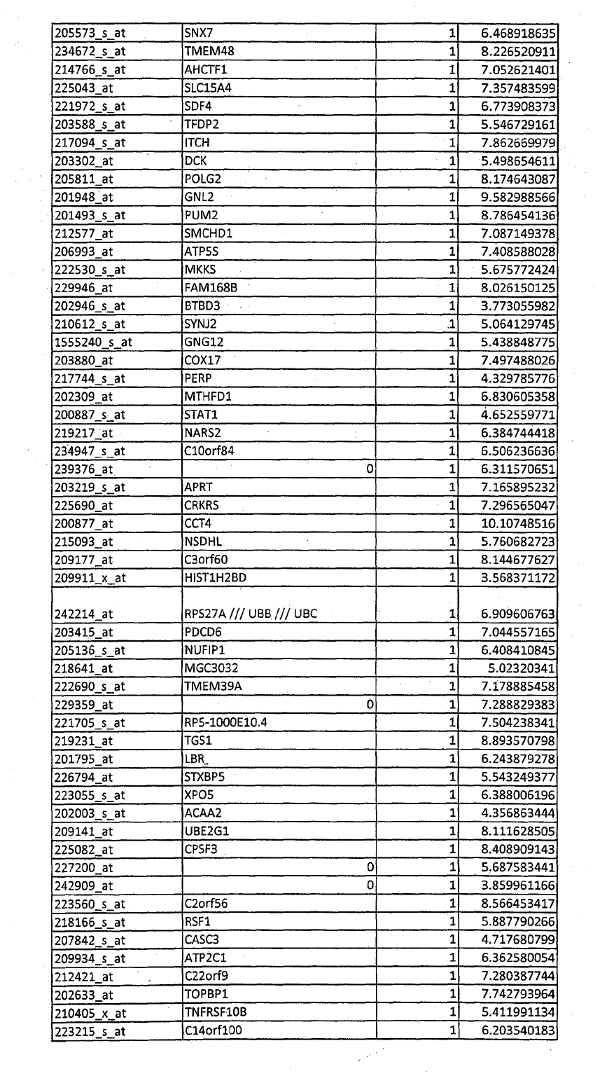

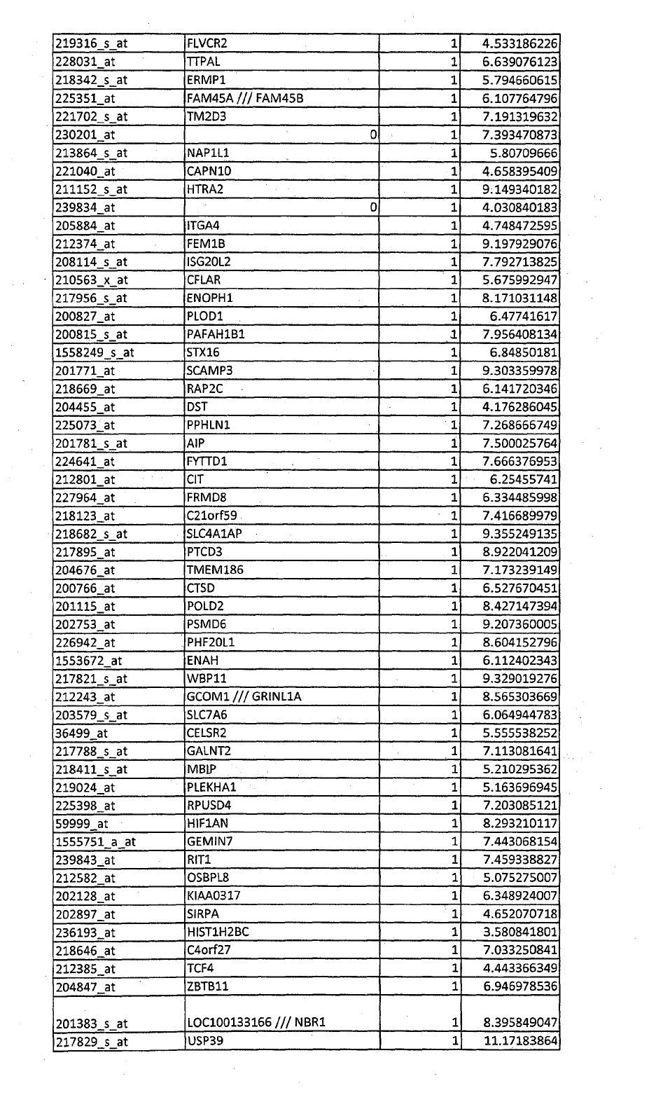

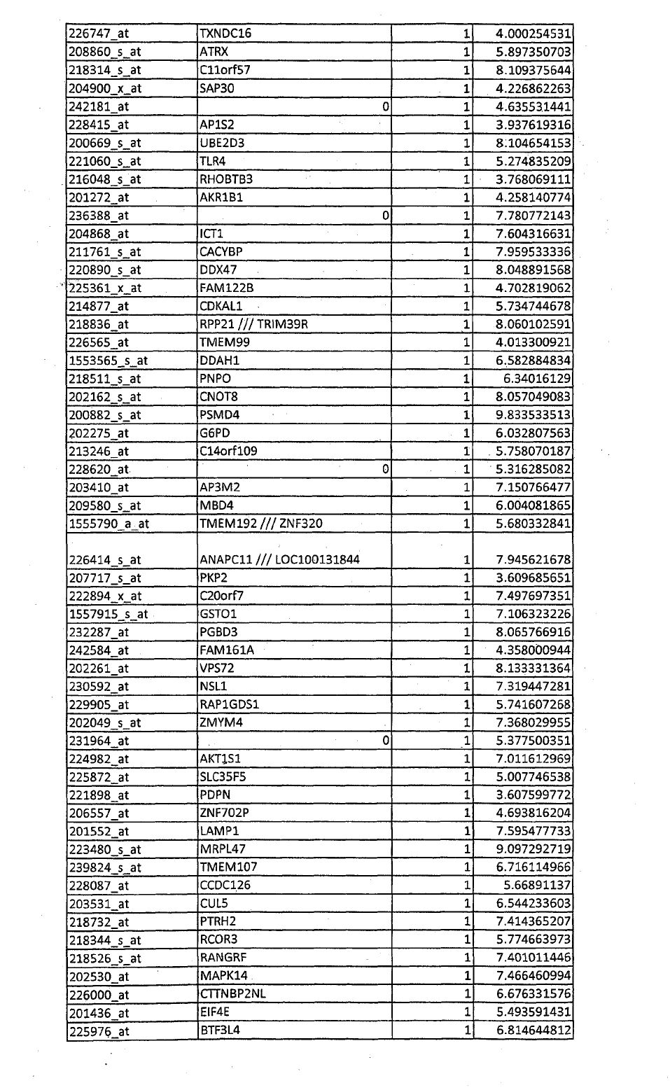

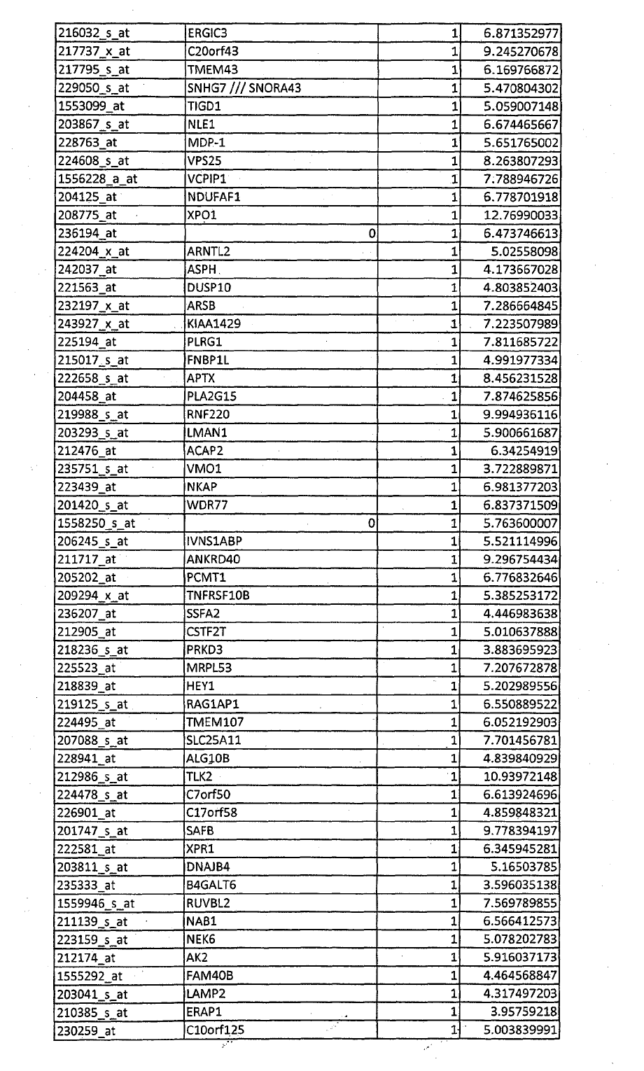

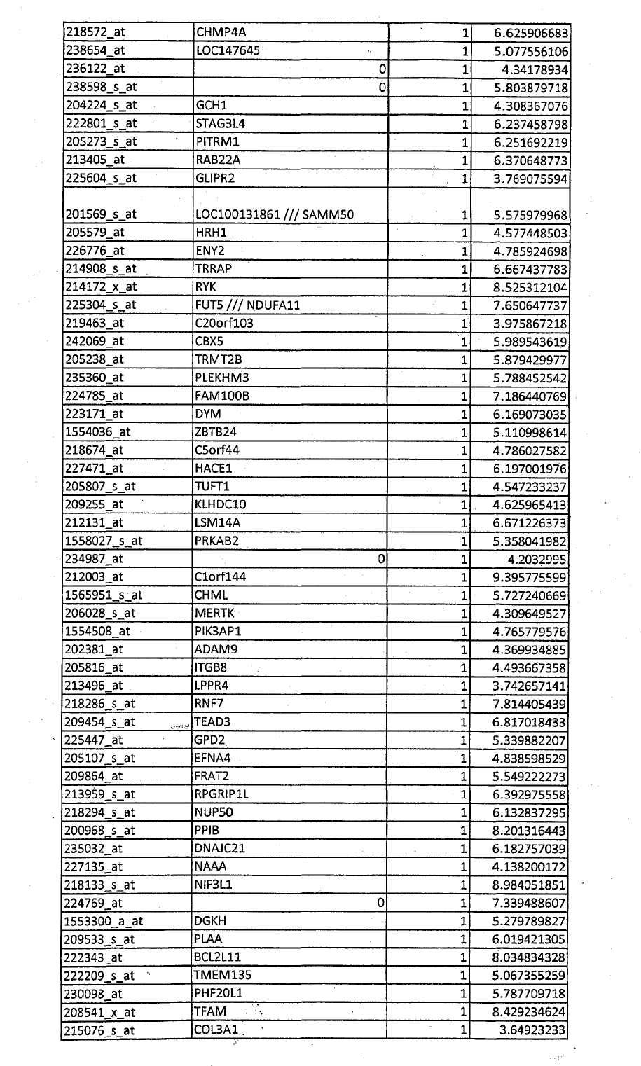

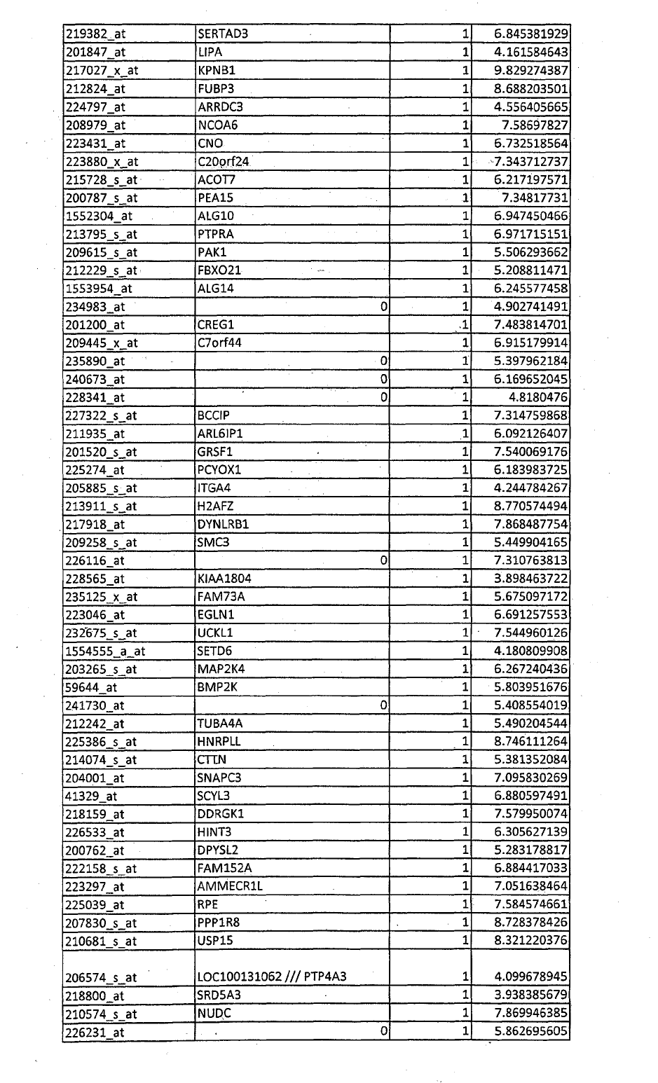

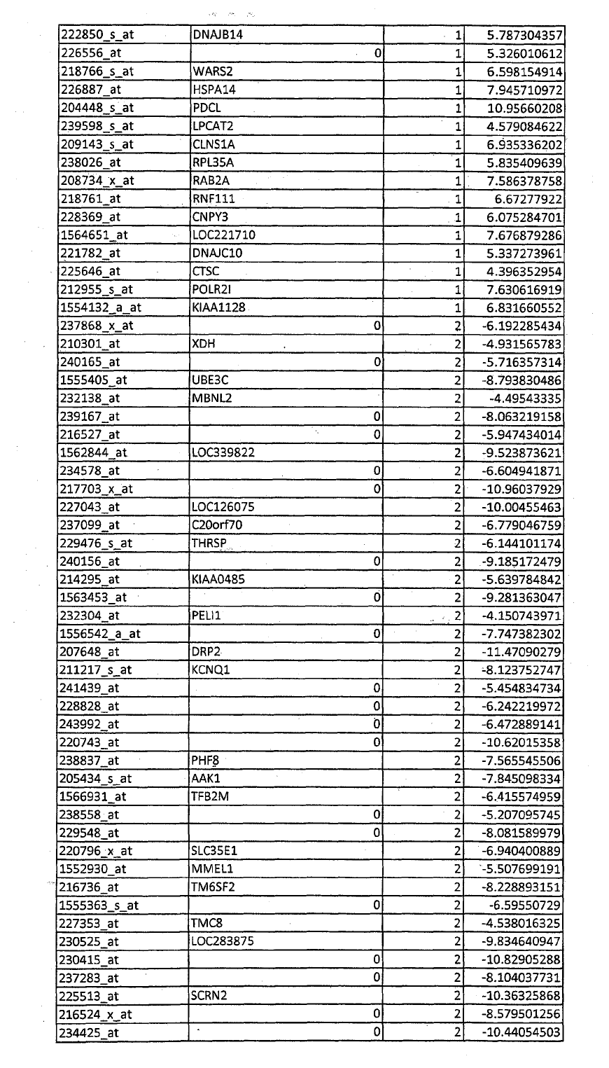

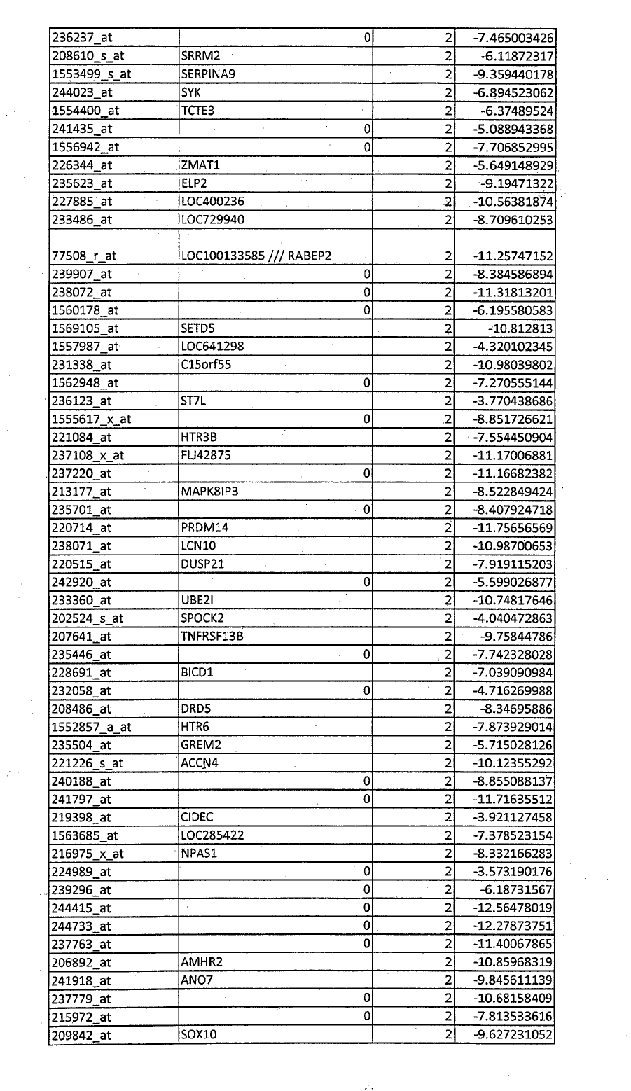

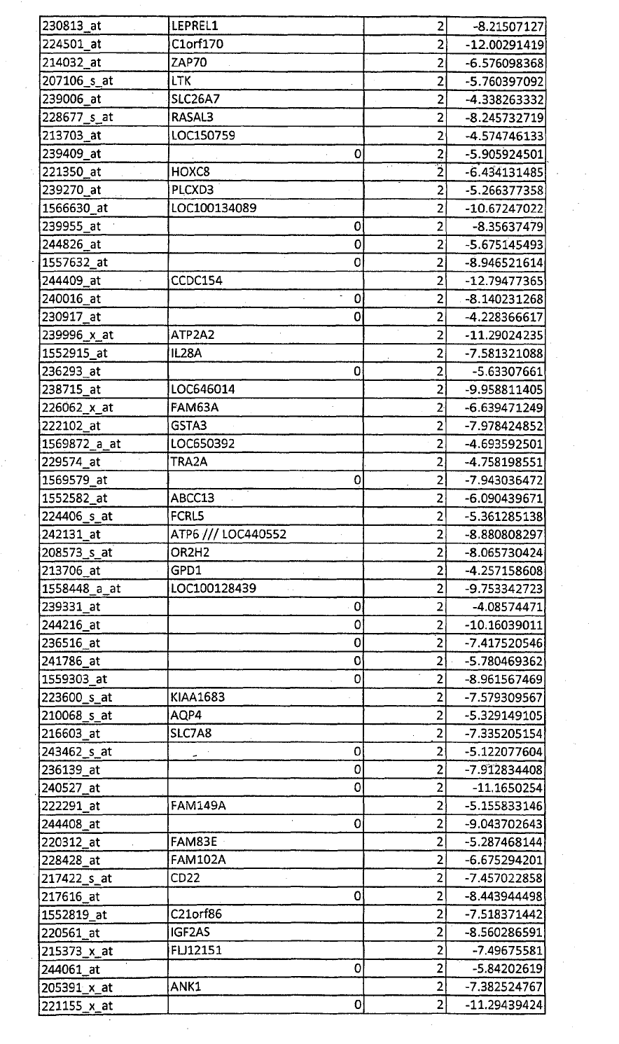

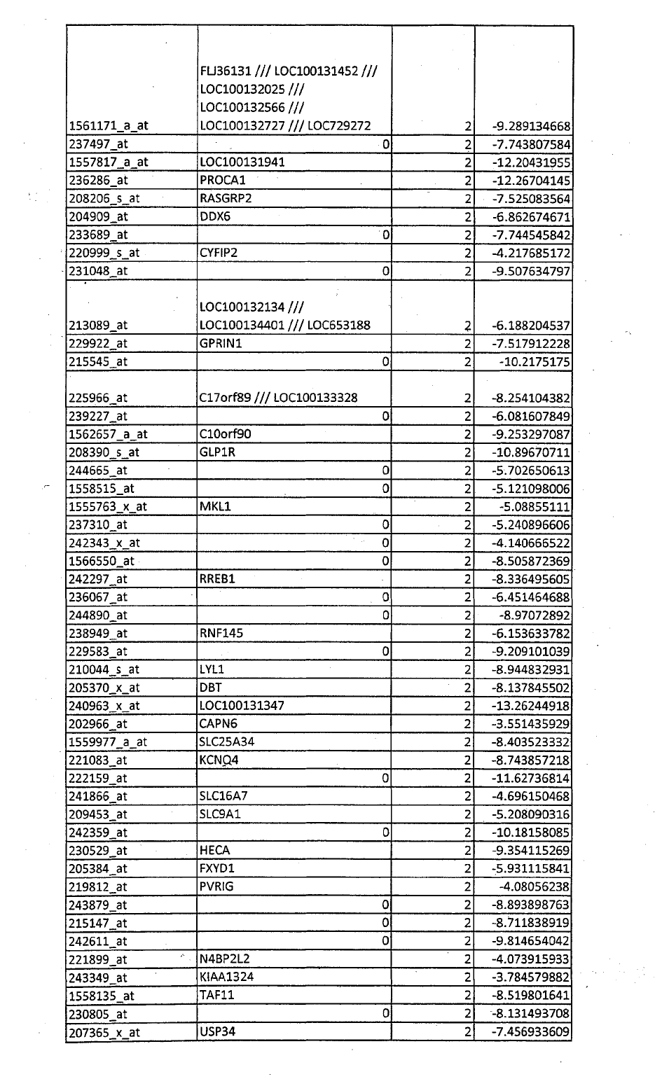

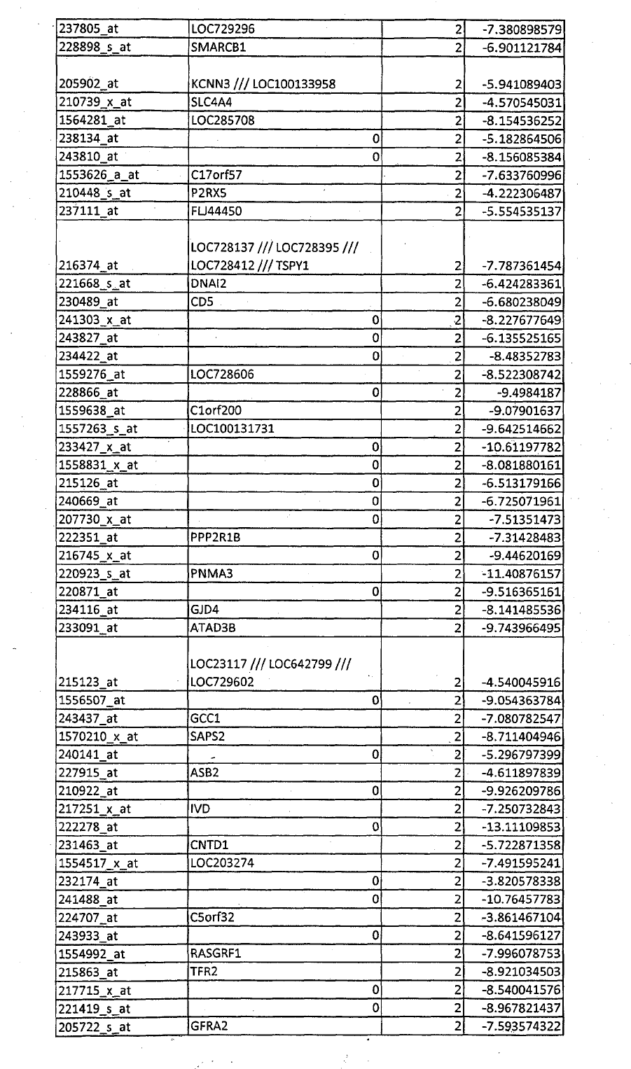

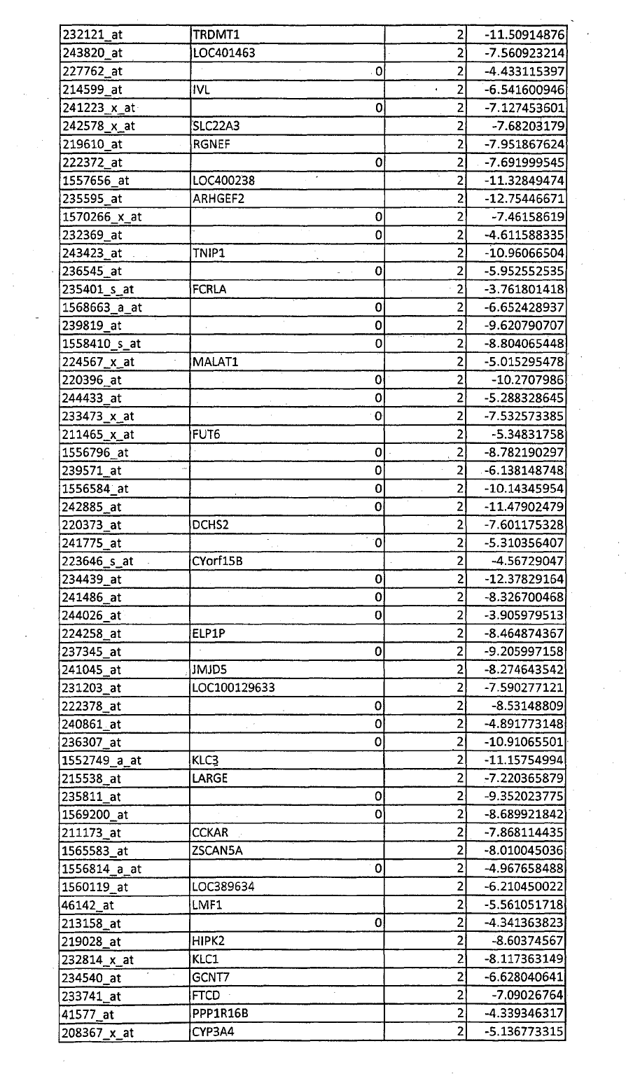

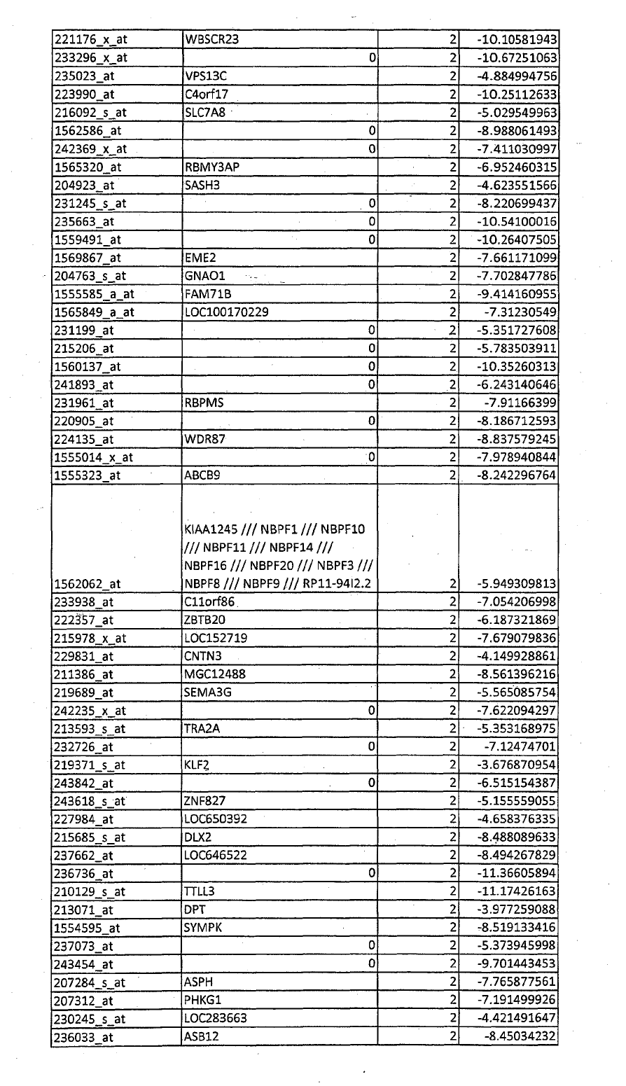

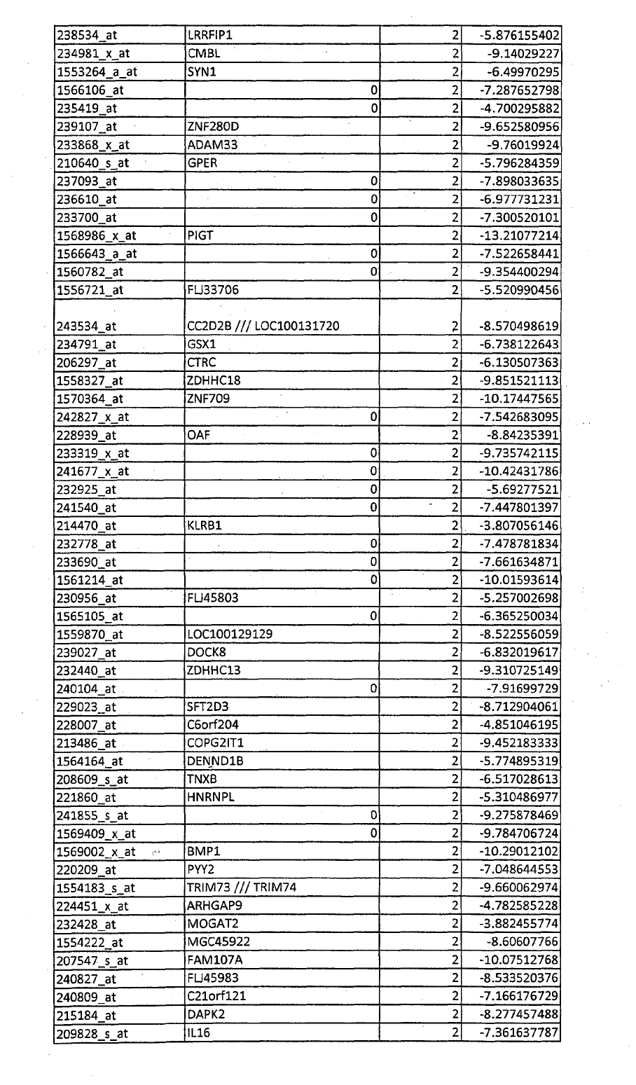

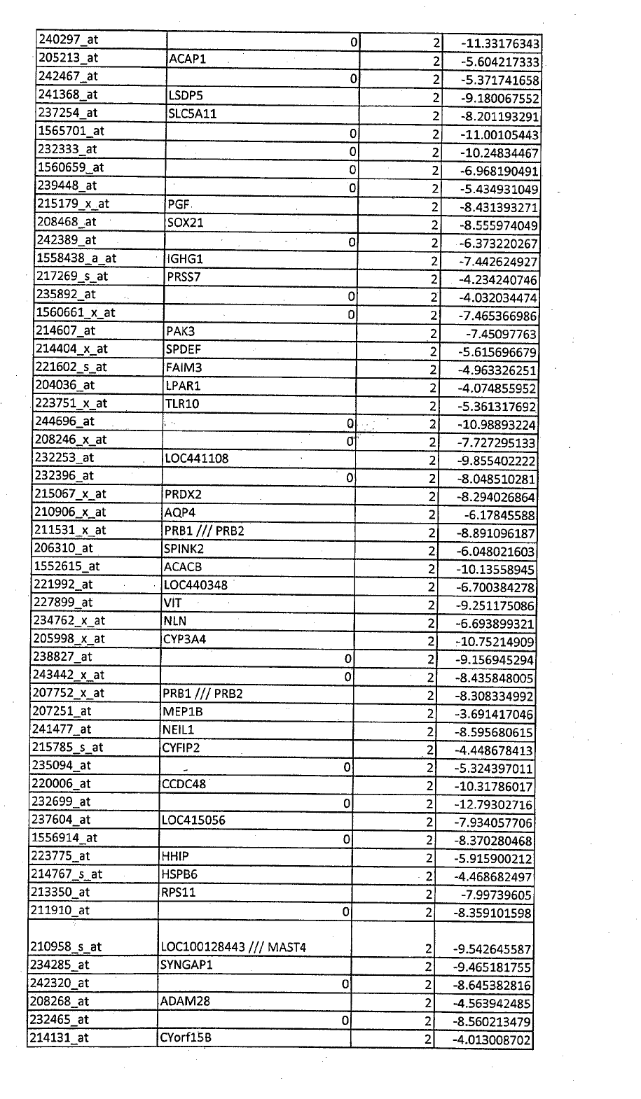

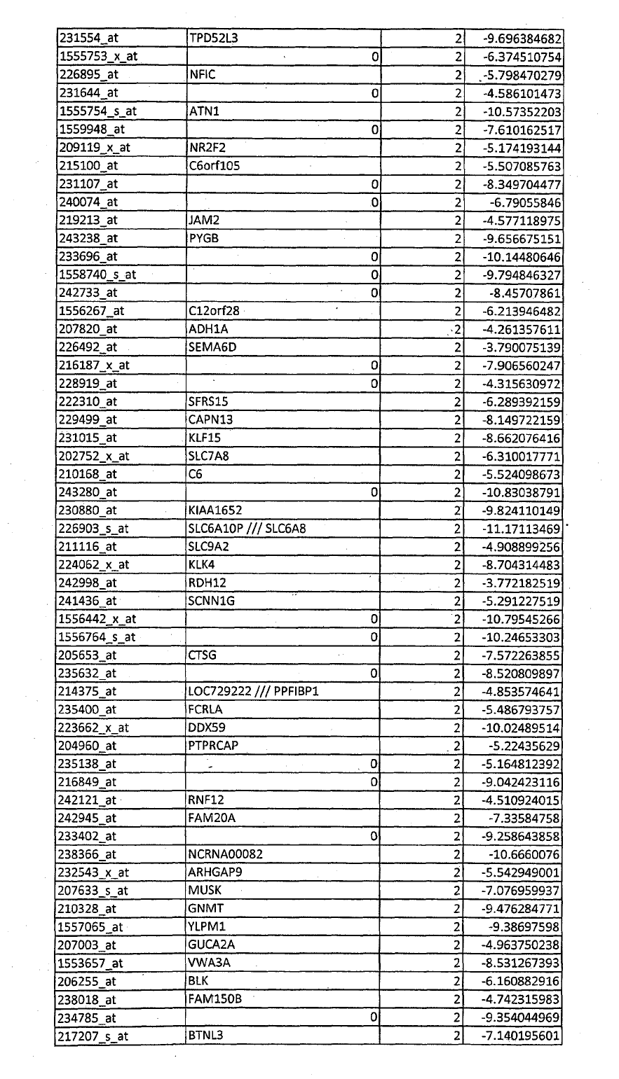

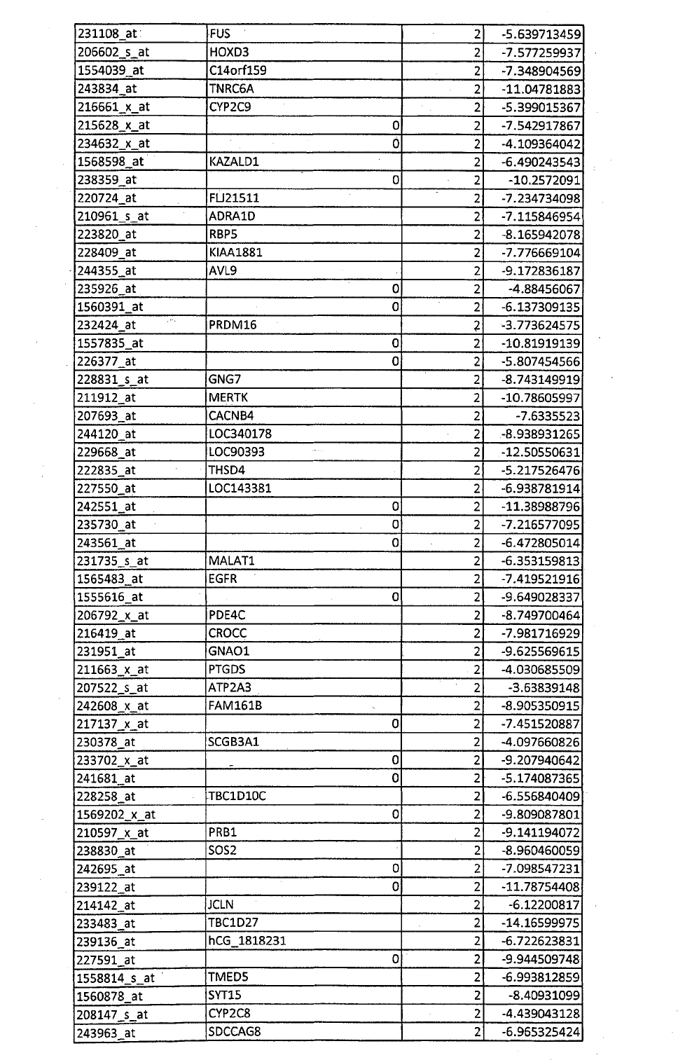

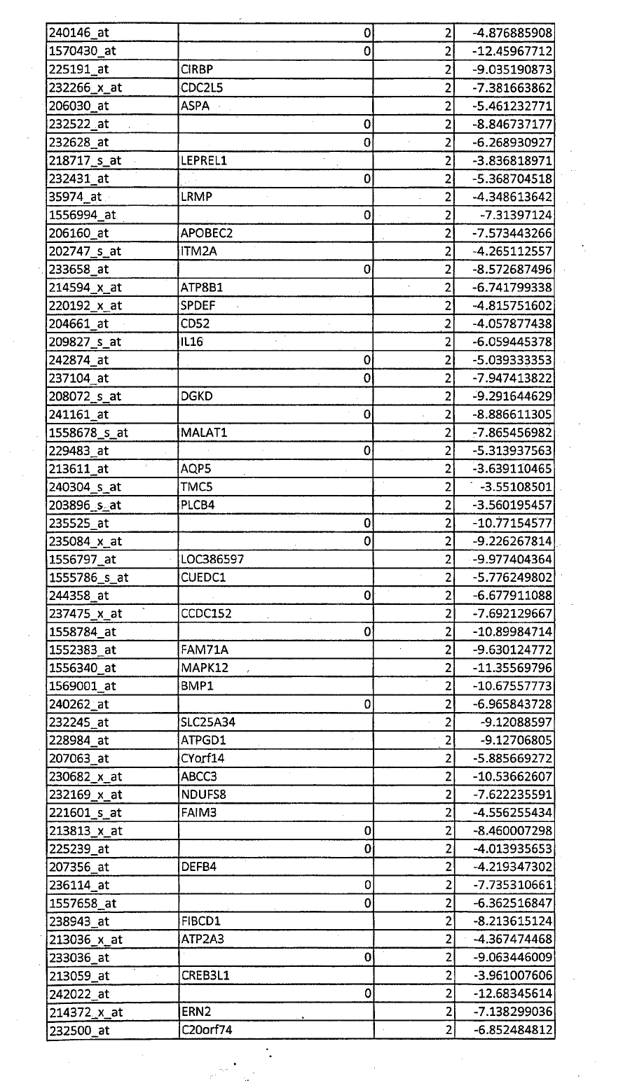

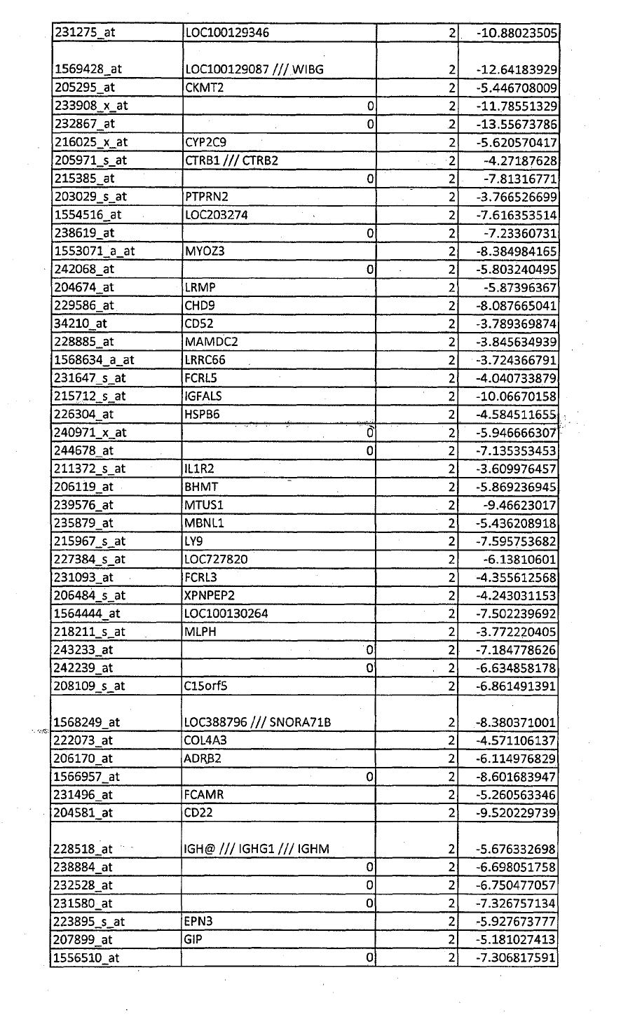

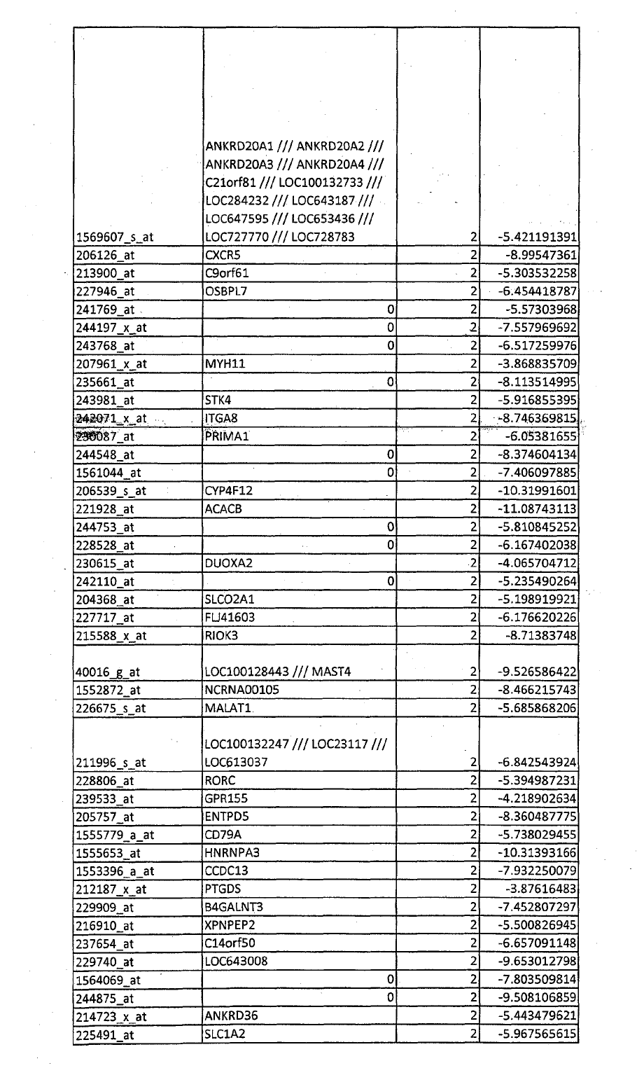

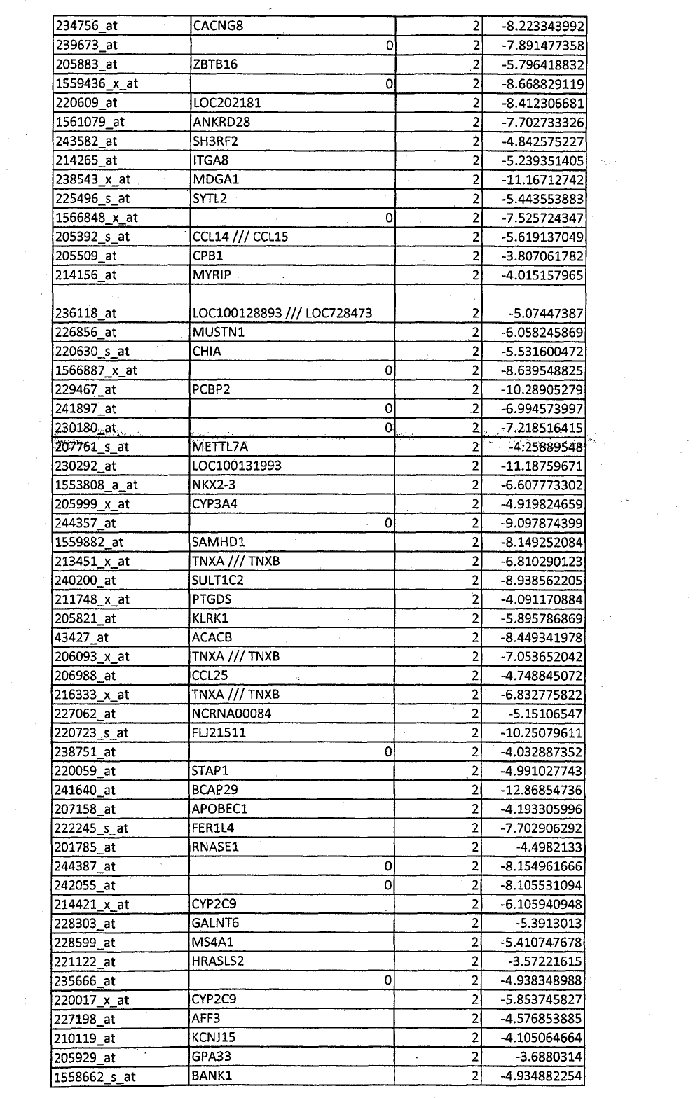

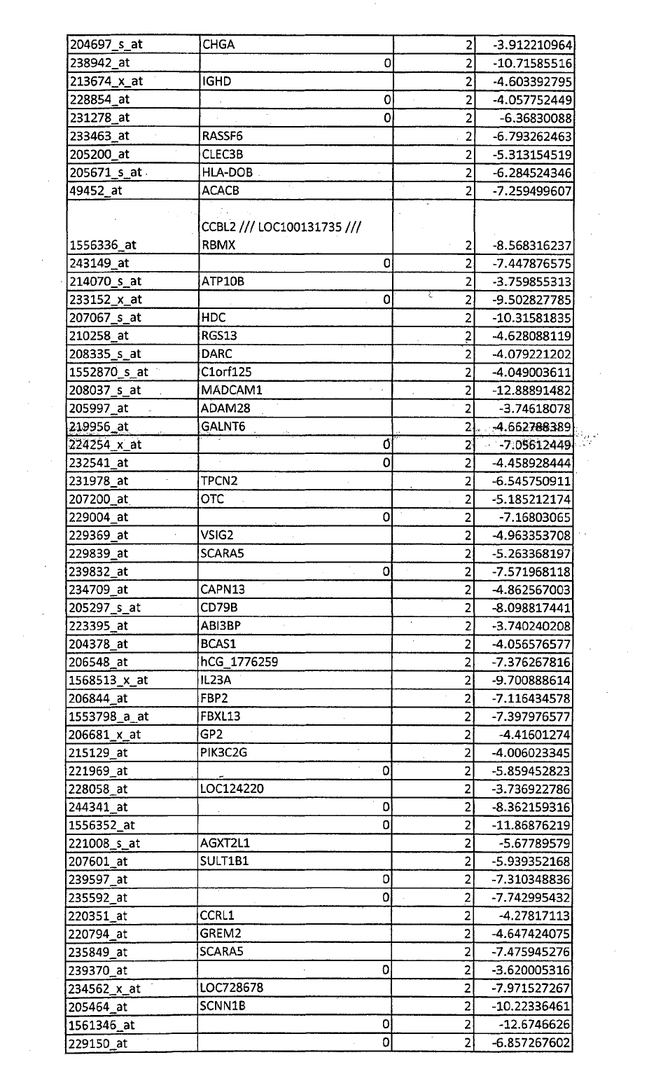

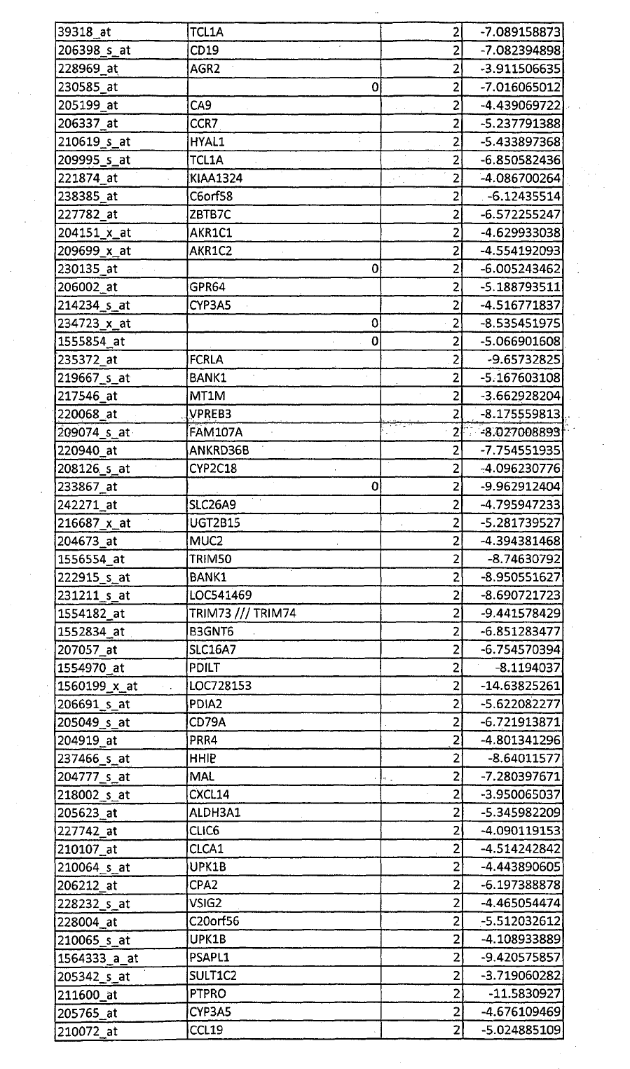

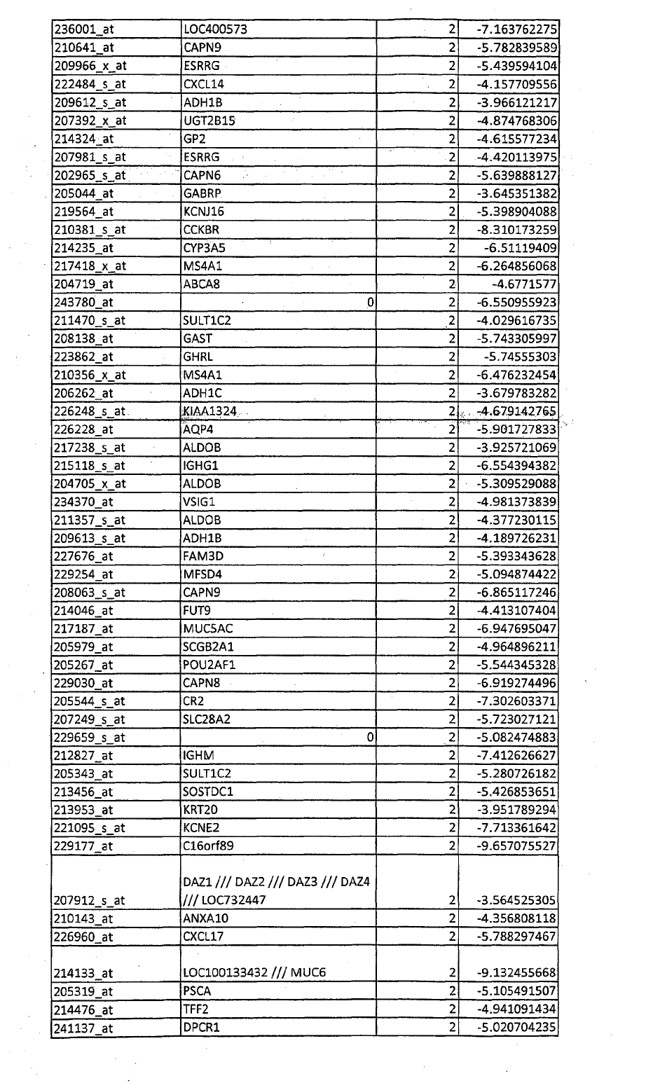

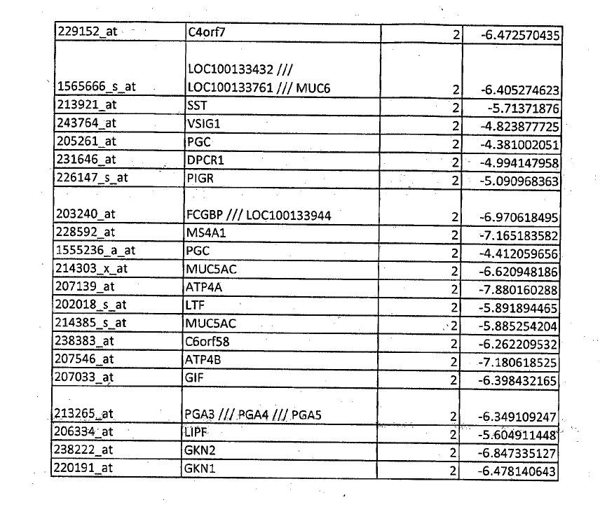

13. The predictor according to claim 8, wherein a third of the three predictors comprises a differentially expressed gene set comparing the differential expression between genes of the proliferative subtype versus the metabolic subtype as shown in FIG. 20.

14. A method of classifying a patient based on the patient's gene expression profile, wherein the patient is suffering or suspected to suffer from gastric cancer, to one of the gastric cancer subtypes referred to in claim 1, wherein the method comprises:
assigning the gene expression profile obtained from the patient to either the invasive subtype, the proliferative subtype or the metabolic subtype when two of the three predictors as defined in claim 8 make the same classification and at least one false discovery rate (FDR) is <0.05.

15. The method according to claim 14, wherein when neither false discovery rate (FDR) is <0.05 or when all three predictors make different classifications, the gene expression profile sample is not classified.

16. A method for predicting response to treatment in a patient with gastric cancer, the method comprising assigning the gene expression profile obtained from a gastric tumor sample from the patient to either the invasive subtype, the proliferative subtype or the metabolic subtype referred to in claim 1 when two of the three predictors as defined in claim 8 make the same classification and at least one false discovery rate (FDR) is <0.05.

17. The method of claim 16, wherein when the gene expression profile is assigned to the metabolic subtype, the patient is responsive to 5-fluorouracil.

18. The method of claim 16, wherein when the gene expression profile is assigned to the invasive subtype, the patient is responsive to compounds selected to inhibit the PI3K/AKT/mTOR pathway.

19. A method of treating a patient suffering or suspected to suffer from gastric cancer, comprising:
administering or recommending or prescribing to the patient an anti-cancer drug, or initiating active treatment, specific for the gastric cancer subtype of the patient referred to in claim 1.

20. A method of treating a patient suffering or suspected to suffer from gastric cancer, comprising:
a. determining the gastric cancer subtype of the patient according to the method of claim 14; and
b. administering or recommending or prescribing to the patient an anti-cancer drug, or initiating active treatment, specific for the gastric cancer subtype of determined in step a.

21. The method of claim 20, wherein where the gastric cancer subtype is determined to be the metabolic subtype, the anti-cancer drug is 5-fluorouracil.

22. The method of claim 20, wherein where the gastric cancer subtype is determined to be the invasive subtype, the anti-cancer drug is selected to inhibit the PI3K/AKT/mTOR pathway.

23.-25. (canceled)

26. The method of claim 18, wherein the compound or anti-cancer drug selected to inhibit the PI3K/AKT/mTOR pathway is selected from the group consisting of: 2-Methyl-2-{4-[3-methyl-2-oxo-8-(quinolin-3-yl)-2,3-dihydro-1H-imidazo[4,5-c]quinolin-1-yl]phenyl}propanenitrile (BEZ235), 4,4′-(6-(2-(difluoromethyl)-1H-benzo[d]imidazol-1-yl)-1,3,5-triazine-2,4-diyl)dimorpholine (ZSTK474), (E)-N′-((6-bromoimidazo[1,2-a]pyridin-3-yl)methylene)-N,2-dimethyl-5-nitrobenzenesulfonohydrazide hydrochloride (PIK-75), 3-[4-(4-Morpholinylpyrido[3′,2′:4,5]furo[3,2-d]pyrimidin-2-yl]phenol hydrochloride (PI-103), 2-Methyl-2-{4-[3-methyl-2-oxo-8-(quinolin-3-yl)-2,3-dihydro-1H-imidazo[4,5-c]quinolin-1-yl]phenyl}propanenitrile (BEZ235), (S)-4-(2-(4-amino-1,2,5-oxadiazol-3-yl)-1-ethyl-7-(piperidin-3-ylmethoxy)-1H-imidazo[4,5-c]pyridin-4-yl)-2-methylbut-3-yn-2-ol (GSK690693), 8-(4-(1-aminocyclobutyl)phenyl)-9-phenyl-8,9-dihydro-[1,2,4]triazolo[3,4-f][1,6]naphthyridin-3(2H)-one dihydrochloride (MK2206), and 6-(2,6-dichlorophenyl)-8-methyl-2-((3-(methylthio)phenyl)amino)pyrido[2,3-d]pyrimidin-7 (8H)-one (PD173955).

27. A computer readable medium having stored therein a computer program comprising a set of executable instructions, when executed by a computer processor, controls the processor to perform the method according to claim 14.

28. A computer program comprising a set of executable instructions, when executed by a computer processor, controls the processor to perform the method according to claim 14.


SEQUENCE LISTING (4 sequences)

SEQ ID NO: 1 - DNA, 401 bp, Homo sapiens
  flank1 (1)..(58): 5′ flanking region of exon 4
cctggtcctctgactgctcgccaggcattgaagtctcatcctggtcctctgactgctcttttcacccatctacagtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacggtcagttgccctgaggggctggcttccatgagacttcaatgcctggc

SEQ ID NO: 2 - DNA, 629 bp, Homo sapiens
  flank1 (1)..(58): 5′ flanking region of exons 5 and 6
ctagctcgctagtgggttgaggagaaagcccccctactgctagctcgctagtgggttgcaggaggtgcttacgcatgtttgtttctttgctgccgtcttccagttgctttatctgttcacttgtgccctgactttcaactctgtctccttcctcttcctacagtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtgagcagctggggctggagagacgacagggctggttgcccagggtccccaggcctctgattcctcactgattgctcttaggtctggcccctcctcagcatcttatccgagtggaaggaaatttgcgtgtggagtatttggatgacagaaacacttttcgacatagtgtggtggtgccctatgagccgcctgaggtctggtttgcaactggggtctctgggaggaggggttaagggtggttgtcagtggccctccaggtgagcagtaggggggctttctcct

SEQ ID NO: 3 - DNA, 437 bp, Homo sapiens
  flank1 (1)..(56): 5′ flanking region of exon 7
tgcttgccacaggtctccaagctccagctccaggtaggtgcttgccacaggtctccccaaggcgcactggcctcatcttgggcctgtgttatctcctaggttggctctgactgtaccaccatccactacaactacatgtgtaacagttcctgcatgggcggcatgaaccggaggcccatcctcaccatcatcacactggaagactccaggtcaggagccacttgccaccctgcacactggcctgctgtgccccagcctctgcttgcctctgacccctgggcccacctcttaccgatttcttccatactactacccatccacctctcatcacatccccggcggggaatctccttactgctcccactcagttttcttttctctggctttgggacctcttaacctgtggcttctcctccacctacctggagctggagctt

SEQ ID NO: 4 - DNA, 449 bp, Homo sapiens
  flank1 (1)..(59): 5′ flanking region of exons 8 and 9
ttccttactgcctcttgcttgaaaacggcattttgagtgttccttactgcctcttgcttctcttttcctatcctgagtagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgaggtaagcaagcaggacaagaagcggtggaggagaccaagggtgcagttatgcctcagattcacttttatcacctttccttgcctctttcctagcactgcccaacaacaccagctcctctccccagccaaagaagaaaccactggatggagaatatttcacccttcaggtactaagtcttgggacctcttatcaagtggaaagtttccagtctaacactcaaaatgccgttttc